SEQUENCE LISTING (847 sequences)

SEQ ID NO: 1 - protein, 4 aa, synthetic construct
  SITE 1: note = D-Tyrosine
  SITE 2: note = D-Tryptophan
YWNT

SEQ ID NO: 2 - protein, 5 aa, synthetic construct
  SITE 1: note = D-Tyrosine
  SITE 2: note = D-Asparagine
  SITE 3: note = D-Arginine
YNRNT

SEQ ID NO: 3 - protein, 4 aa, synthetic construct
  SITE 1: note = D-Tyrosine
YRNT

SEQ ID NO: 4 - protein, 4 aa, synthetic construct
  SITE 1: note = D-Tyrosine
YKNT

SEQ ID NO: 5-6 - skipped (2 entries)

SEQ ID NO: 7 - protein, 4 aa, synthetic construct
  SITE 1: note = D-Tyrosine
YENT

SEQ ID NO: 8 - protein, 4 aa, synthetic construct
  SITE 1: note = D-Tyrosine
  SITE 2: note = D-Alanine
YANT

SEQ ID NO: 9 - protein, 4 aa, synthetic construct
  SITE 1: note = D-Tyrosine
YWNA

SEQ ID NO: 10 - protein, 4 aa, synthetic construct
  SITE 1: note = D-Tyrosine
YWNT

SEQ ID NO: 11 - protein, 5 aa, synthetic construct
  SITE 1: note = D-Tyrosine
  SITE 3: note = D-Tryptophan
YNWNT

SEQ ID NO: 12 - protein, 5 aa, synthetic construct
  SITE 1: note = D-Tyrosine
  SITE 2: note = D-Histidine
  SITE 3: note = D-Tryptophan
YHWNT

SEQ ID NO: 13 - protein, 5 aa, synthetic construct
  SITE 1: note = D-Alanine
ANWNG

SEQ ID NO: 14 - protein, 4 aa, synthetic construct
  SITE 1: note = D-Tryptophan
WNTF

SEQ ID NO: 15 - protein, 5 aa, synthetic construct
  SITE 1: note = D-Alanine
ANWNS

SEQ ID NO: 16 - protein, 4 aa, synthetic construct
  SITE 1: note = D-Tyrosine
  SITE 2: note = Azaglycine
YGNT

SEQ ID NO: 17 - protein, 5 aa, synthetic construct
  SITE 1: note = D-Asparagine
  SITE 2: note = D-Asparagine
  SITE 3: note = D-Asparagine
  SITE 4: note = D-Asparagine
  SITE 5: note = D-Asparagine
NNNNN

SEQ ID NO: 18 - protein, 5 aa, synthetic construct
  SITE 1: note = D-Asparagine
  SITE 2: note = D-Asparagine
  SITE 3: note = D-Glutamic Acid
  SITE 4: note = D-Glutamic Acid
  SITE 5: note = D-Asparagine
NNEEN

SEQ ID NO: 19 - protein, 5 aa, synthetic construct
  SITE 1: note = D-Asparagine
  SITE 2: note = D-Asparagine
  SITE 3: note = D-Lysine
  SITE 4: note = D-Glutamic Acid
  SITE 5: note = D-Asparagine
NNKEN

SEQ ID NO: 20 - protein, 4 aa, synthetic construct
  SITE 1: note = D-Tyrosine
YPNT

SEQ ID NO: 21 - protein, 4 aa, synthetic construct
  SITE 1: note = 2-(2-(2-hydroxyethoxy)ethoxy)acetic acid coupled to N-terminus of D-Tyrosine
  SITE 2: note = Lysine bound to an optional linker and chelating moiety or a radionuclide complex
YKNT

SEQ ID NO: 22 - protein, 7 aa, synthetic construct
  SITE 1: note = palmitic acid coupled to N-terminus of Glutamic Acid
  SITE 2: note = Lysine bound to an optional linker and chelating moiety or a radionuclide complex
EKYNWNS

SEQ ID NO: 23 - protein, 7 aa, synthetic construct
  SITE 1: note = Acetyl coupled to N-terminus of Glutamic Acid
  SITE 2: note = Lysine bound to an optional linker and chelating moiety or a radionuclide complex
  SITE 3: note = D-Alanine
EKANWNG

SEQ ID NO: 24 - protein, 4 aa, synthetic construct
  SITE 1: note = Sarcosine
  SITE 2: note = Sarcosine
  SITE 3: note = Sarcosine
  SITE 4: note = Sarcosine
GGGG

SEQ ID NO: 25 - protein, 5 aa, synthetic construct
  SITE 1: note = Sarcosine
  SITE 2: note = Sarcosine
  SITE 3: note = Sarcosine
  SITE 4: note = Sarcosine
  SITE 5: note = Sarcosine
GGGGG

SEQ ID NO: 26 - protein, 5 aa, synthetic construct
  SITE 1: note = D-Alanine
  SITE 2: note = D-Alanine
  SITE 3: note = D-Alanine
  SITE 4: note = D-Alanine
  SITE 5: note = D-Alanine
AAAAA

SEQ ID NO: 27 - skipped

SEQ ID NO: 28 - protein, 4 aa, synthetic construct
KWNT

SEQ ID NO: 29 - protein, 4 aa, synthetic construct
  SITE 1: note = 4-iodophenylalanine
FWNT

SEQ ID NO: 30 - protein, 4 aa, synthetic construct
  SITE 1: note = D-4-iodophenylalanine
  SITE 2: note = D-Tryptophan
FWNT

SEQ ID NO: 31 - skipped

SEQ ID NO: 32 - protein, 4 aa, synthetic construct
  SITE 2: note = Azaglycine
YGNT

SEQ ID NO: 33-37 - skipped (5 entries)

SEQ ID NO: 38 - protein, 4 aa, synthetic construct
  SITE 1: note = D-Tyrosine
  SITE 2: note = Lysine(1,4,7,10-tetraazacyclododecane-1,4,7,10-tetr aacetic acid)
YKNT

SEQ ID NO: 39 - protein, 4 aa, synthetic construct
  SITE 2: note = D-Lysine(1,4,7,10-tetraazacyclododecane-1,4,7,10-te traacetic acid)
YKNT

SEQ ID NO: 40 - protein, 4 aa, synthetic construct
  SITE 1: note = D-Tyrosine
  SITE 2: note = D-Lysine(1,4,7,10-tetraazacyclododecane-1,4,7,10-te traacetic acid)
YKNT

SEQ ID NO: 41-44 - skipped (4 entries)

SEQ ID NO: 45 - protein, 4 aa, synthetic construct
  SITE 1: note = D-Tyrosine
  SITE 2: note = 4-iodophenylalanine
YFNT

SEQ ID NO: 46 - protein, 4 aa, synthetic construct
YPNT

SEQ ID NO: 47 - protein, 4 aa, synthetic construct
  SITE 1: note = D-Tyrosine
  SITE 2: note = D-Tryptophan
YWNA

SEQ ID NO: 48 - protein, 5 aa, synthetic construct
  SITE 1: note = gamma-D-Glutamic Acid
  SITE 2: note = D-Tyrosine
  SITE 3: note = Lysine(1,4,7,10-tetraazacyclododecane-1,4,7,10-tetr aacetic acid)
EYKNT

SEQ ID NO: 49 - protein, 5 aa, synthetic construct
  SITE 2: note = D-Tyrosine
  SITE 3: note = 3-(2-naphthyl)alanine
GYXNT

SEQ ID NO: 50 - protein, 5 aa, synthetic construct
  SITE 3: note = D-Tryptophan
GYWNT

SEQ ID NO: 51 - protein, 5 aa, synthetic construct
YNWNS

SEQ ID NO: 52 - protein, 5 aa, synthetic construct
  SITE 5: note = 3-fluorophenylalanine
YENTF

SEQ ID NO: 53 - protein, 5 aa, synthetic construct
  SITE 2: note = D-Tryptophan
  SITE 5: note = 3-fluorophenylalanine
YWNTF

SEQ ID NO: 54 - protein, 5 aa, synthetic construct
  SITE 1: note = D-Tyrosine
  SITE 2: note = D-Tryptophan
  SITE 5: note = 3-fluorophenylalanine
YWNTF

SEQ ID NO: 55 - protein, 9 aa, synthetic construct
  SITE 1: note = D-Tyrosine
  SITE 2: note = D-Tryptophan
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YWNTFGLXW

SEQ ID NO: 56 - protein, 5 aa, synthetic construct
  SITE 2: note = Azaglycine
  SITE 4: note = 4-amino-1-((3-fluoro-4-methylbenzyl)glycyl)piperidi ne-4-carboxamide
  SITE 5: note = Amidated Tryptophan
FGLXW

SEQ ID NO: 57 - protein, 5 aa, synthetic construct
  SITE 4: note = 4-amino-1-carbamimidoylpiperidine-4-carboxylic acid
  SITE 5: note = Amidated Tryptophan
FGLXW

SEQ ID NO: 58 - protein, 5 aa, synthetic construct
  SITE 1: note = alpha-Me-Phenylalanine
  SITE 2: note = Azaglycine
  SITE 4: note = 4-amino-1-((3-fluoro-4-methylbenzyl)glycyl)piperidi ne-4-carboxamide
  SITE 5: note = Amidated Tryptophan
FGLXW

SEQ ID NO: 59 - protein, 5 aa, synthetic construct
  SITE 2: note = Azaglycine
  SITE 4: note = 4-amino-1-carbamimidoylpiperidine-4-carboxylic acid
  SITE 5: note = Amidated Tryptophan
FGLXW

SEQ ID NO: 60 - protein, 9 aa, synthetic construct
  SITE 1: note = D-Tyrosine
  SITE 2: note = D-Tryptophan
  SITE 6: note = Azaglycine
  SITE 8: note = 4-amino-1-carbamimidoylpiperidine-4-carboxylic acid
  SITE 9: note = Amidated Tryptophan
YWNTFGLXW

SEQ ID NO: 61 - protein, 5 aa, synthetic construct
  SITE 2: note = Azaglycine
  SITE 4: note = 4-amino-1-(4-(p-tolyl)butanoyl)piperidine-4-carboxy lic acid
  SITE 5: note = Amidated Tryptophan
FGLXW

SEQ ID NO: 62 - skipped

SEQ ID NO: 63 - protein, 5 aa, synthetic construct
  SITE 4: note = 4-amino-1-((3-fluoro-4-methylbenzyl)glycyl)piperidi ne-4-carboxamide
  SITE 5: note = Amidated Phenylalanine
FGLXF

SEQ ID NO: 64 - protein, 5 aa, synthetic construct
  SITE 2: note = Sarcosine
  SITE 4: note = 4-amino-1-((3-fluoro-4-methylbenzyl)glycyl)piperidi ne-4-carboxamide
  SITE 5: note = Amidated Tryptophan
FGLXW

SEQ ID NO: 65 - protein, 10 aa, synthetic construct
  SITE 1: note = D-Asparagine
  SITE 2: note = D-Asparagine
  SITE 3: note = D-Glutamic Acid
  SITE 4: note = D-Glutamic Acid
  SITE 5: note = D-Asparagine
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
NNEENFGLXW

SEQ ID NO: 66 - protein, 10 aa, synthetic construct
  SITE 1: note = D-Asparagine
  SITE 2: note = D-Asparagine
  SITE 3: note = D-Lysine
  SITE 4: note = D-Glutamic Acid
  SITE 5: note = D-Asparagine
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
NNKENFGLXW

SEQ ID NO: 67 - protein, 7 aa, synthetic construct
  SITE 1: note = D-Asparagine
  SITE 2: note = D-Asparagine
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
NNFGLXW

SEQ ID NO: 68 - protein, 7 aa, synthetic construct
  SITE 1: note = D-Lysine
  SITE 2: note = D-Asparagine
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
KNFGLXW

SEQ ID NO: 69 - protein, 9 aa, synthetic construct
  SITE 1: note = D-Tyrosine
  SITE 2: note = hydroxyproline
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YXNTFGLXW

SEQ ID NO: 70 - protein, 6 aa, synthetic construct
  SITE 1: note = D-Asparagine
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
NFGLXW

SEQ ID NO: 71 - protein, 9 aa, synthetic construct
  SITE 1: note = D-Tyrosine
  SITE 2: note = D-3-(4-pyridyl)alanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YXNTFGLXW

SEQ ID NO: 72 - protein, 9 aa, synthetic construct
  SITE 1: note = D-Tyrosine
  SITE 5: note = 3-Fluorophenylalanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YENTFGLXW

SEQ ID NO: 73 - protein, 7 aa, synthetic construct
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
NTFGLXW

SEQ ID NO: 74 - protein, 9 aa, synthetic construct
  SITE 1: note = D-Tyrosine
  SITE 5: note = 3-Fluorophenylalanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YKNTFGLXW

SEQ ID NO: 75 - protein, 9 aa, synthetic construct
  SITE 1: note = D-Tyrosine
  SITE 2: note = 3-(4-pyridyl)alanine
  SITE 5: note = 3-Fluorophenylalanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YXNTFGLXW

SEQ ID NO: 76 - protein, 8 aa, synthetic construct
  SITE 1: note = D-Tyrosine
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
YNTFGLXW

SEQ ID NO: 77 - protein, 10 aa, synthetic construct
  SITE 1: note = D-Tyrosine
  SITE 2: note = D-Histidine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
YHWNTFGLXW

SEQ ID NO: 78 - protein, 9 aa, synthetic construct
  SITE 1: note = D-Tyrosine
  SITE 5: note = 3-Fluorophenylalanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YWNTFGLXW

SEQ ID NO: 79 - protein, 9 aa, synthetic construct
  SITE 1: note = D-Tyrosine
  SITE 5: note = 3-Fluorophenylalanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YENTFGLXW

SEQ ID NO: 80 - protein, 5 aa, synthetic construct
  SITE 2: note = Azaglycine
  SITE 3: note = D-Leucine
  SITE 4: note = 4-amino-1-((3-fluoro-4-methylbenzyl)glycyl)piperidi ne-4-carboxamide
  SITE 5: note = Amidated Tryptophan
FGLXW

SEQ ID NO: 81 - protein, 5 aa, synthetic construct
  SITE 3: note = D-Leucine
  SITE 4: note = 4-amino-1-carbamimidoylpiperidine-4-carboxylic acid
  SITE 5: note = Amidated Tryptophan
FGLXW

SEQ ID NO: 82 - protein, 5 aa, synthetic construct
  SITE 2: note = Sarcosine
  SITE 3: note = Methyl-leucine
  SITE 4: note = 4-amino-1-((3-fluoro-4-methylbenzyl)glycyl)piperidi ne-4-carboxamide
  SITE 5: note = Amidated Tryptophan
FGLXW

SEQ ID NO: 83 - protein, 5 aa, synthetic construct
  SITE 2: note = Azaglycine
  SITE 4: note = 4-amino-1-((3-fluoro-4-methylbenzyl)glycyl)piperidi ne-4-carboxamide
  SITE 5: note = Amidated Methyl-tryptophan
FGLXW

SEQ ID NO: 84-86 - skipped (3 entries)

SEQ ID NO: 87 - protein, 5 aa, synthetic construct
  SITE 3: note = Methyl-leucine
  SITE 4: note = 4-amino-1-((3-fluoro-4-methylbenzyl)glycyl)piperidi ne-4-carboxamide
  SITE 5: note = Amidated Tryptophan
FGLXW

SEQ ID NO: 88 - skipped

SEQ ID NO: 89 - protein, 5 aa, synthetic construct
  SITE 2: note = Azaglycine
  SITE 4: note = 4-amino-1-((3-fluoro-4-methylbenzyl)glycyl)piperidi ne-4-carboxamide
  SITE 5: note = Amidated Phenylalanine
FGLXF

SEQ ID NO: 90 - protein, 9 aa, synthetic construct
  SITE 1: note = D-Tyrosine
  SITE 2: note = D-Tryptophan
  SITE 6: note = Azaglycine
  SITE 8: note = 4-amino-1-((3-fluoro-4-methylbenzyl)glycyl)piperidi ne-4-carboxamide
  SITE 9: note = Amidated Phenylalanine
YWNTFGLXF

SEQ ID NO: 91 - protein, 9 aa, synthetic construct
  SITE 1: note = D-Tyrosine
  SITE 2: note = 3-(2-Naphthyl)alanine or
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YXNTFGLXW

SEQ ID NO: 92 - protein, 9 aa, synthetic construct
  SITE 1: note = D-Tyrosine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YRNTFGLXW

SEQ ID NO: 93 - protein, 10 aa, synthetic construct
  SITE 1: note = D-Tyrosine
  SITE 2: note = D-Asparagine
  SITE 3: note = D-Arginine
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
YNRNTFGLXW

SEQ ID NO: 94 - protein, 7 aa, synthetic construct
  SITE 1: note = D-Glutamic Acid
  SITE 2: note = D-Asparagine
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
ENFGLXW

SEQ ID NO: 95 - protein, 9 aa, synthetic construct
  SITE 1: note = D-Tyrosine
  SITE 2: note = D-Alanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YANTFGLXW

SEQ ID NO: 96 - protein, 9 aa, synthetic construct
  SITE 1: note = D-Tyrosine
  SITE 2: note = Lysine bound to an optional linker and chelating moiety or a radionuclide complex
  SITE 5: note = 3-Fluorophenylalanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YKNTFGLXW

SEQ ID NO: 97 - protein, 9 aa, synthetic construct
  SITE 1: note = D-Tyrosine
  SITE 2: note = D-Tryptophan
  SITE 6: note = Azaglycine
  SITE 8: note = 4-amino-1-(4-(3-fluoro-4-methylphenyl)butanoyl)pipe ridine-4-carboxylic acid
  SITE 9: note = Amidated Tryptophan
YWNTFGLXW

SEQ ID NO: 98 - protein, 9 aa, synthetic construct
  SITE 1: note = D-Tyrosine
  SITE 2: note = D-Tryptophan
  SITE 6: note = Azaglycine
  SITE 8: note = 4-amino-1-(4-(p-tolyl)butanoyl)piperidine-4-carboxy lic acid
  SITE 9: note = Amidated Tryptophan
YWNTFGLXW

SEQ ID NO: 99 - protein, 9 aa, synthetic construct
  SITE 1: note = D-Tyrosine
  SITE 2: note = D-Tryptophan
  SITE 6: note = Azaglycine
  SITE 8: note = 4-amino-1-(2-(p-tolyl)acetyl)piperidine-4-carboxyli c acid
  SITE 9: note = Amidated Tryptophan
YWNTFGLXW

SEQ ID NO: 100 - protein, 9 aa, synthetic construct
  SITE 1: note = D-Tyrosine
  SITE 2: note = D-Tryptophan
  SITE 6: note = Azaglycine
  SITE 8: note = 4-amino-1-(2-(4-iodophenyl)acetyl)piperidine-4-carb oxylic acid
  SITE 9: note = Amidated Tryptophan
YWNTFGLXW

SEQ ID NO: 101 - protein, 9 aa, synthetic construct
  SITE 1: note = D-Tyrosine
  SITE 2: note = D-Tryptophan
  SITE 6: note = Azaglycine
  SITE 8: note = 4-amino-1-((4-aminobenzyl)glycyl)piperidine-4-carbo xylic acid
  SITE 9: note = Amidated Tryptophan
YWNTFGLXW

SEQ ID NO: 102 - protein, 9 aa, synthetic construct
  SITE 1: note = D-Tyrosine
  SITE 2: note = D-Tryptophan
  SITE 6: note = Azaglycine
  SITE 8: note = 4-amino-1-(2-amino-2-(3-fluoro-4-methylphenyl)acety l)piperidine-4-carboxylic acid
  SITE 9: note = Amidated Tryptophan
YWNTFGLXW

SEQ ID NO: 103 - protein, 9 aa, synthetic construct
  SITE 1: note = D-Tyrosine
  SITE 2: note = D-Tryptophan
  SITE 6: note = Azaglycine
  SITE 8: note = 4-amino-1-(N-(2-aminoethyl)-N-methylglycyl)piperidi ne-4-carboxylic acid
  SITE 9: note = Amidated Tryptophan
YWNTFGLXW

SEQ ID NO: 104 - protein, 9 aa, synthetic construct
  SITE 1: note = D-Tyrosine
  SITE 2: note = D-Tryptophan
  SITE 6: note = Azaglycine
  SITE 8: note = 4-amino-1-((4-cyclopropylbenzyl)glycyl)piperidine-4 -carboxylic acid
  SITE 9: note = Amidated Tryptophan
YWNTFGLXW

SEQ ID NO: 105 - protein, 9 aa, synthetic construct
  SITE 1: note = D-Tyrosine
  SITE 2: note = D-Tryptophan
  SITE 6: note = Azaglycine
  SITE 8: note = 4-amino-1-((4-ethylbenzyl)glycyl)piperidine-4-carbo xylic acid
  SITE 9: note = Amidated Tryptophan
YWNTFGLXW

SEQ ID NO: 106 - protein, 9 aa, synthetic construct
  SITE 1: note = D-Tyrosine
  SITE 2: note = D-Tryptophan
  SITE 6: note = Azaglycine
  SITE 8: note = 4-amino-1-((4-hydroxybenzyl)glycyl)piperidine-4-car boxylic acid
  SITE 9: note = Amidated Tryptophan
YWNTFGLXW

SEQ ID NO: 107 - protein, 9 aa, synthetic construct
  SITE 1: note = D-Tyrosine
  SITE 2: note = D-Tryptophan
  SITE 6: note = Azaglycine
  SITE 8: note = 4-amino-1-((3,5-difluoro-4-hydroxybenzyl)glycyl)pip eridine-4-carboxylic acid
  SITE 9: note = Amidated Tryptophan
YWNTFGLXW

SEQ ID NO: 108 - protein, 9 aa, synthetic construct
  SITE 1: note = D-Tyrosine
  SITE 2: note = D-Tryptophan
  SITE 6: note = Azaglycine
  SITE 8: note = (R)-4-amino-1-((R)-(1-(3-fluoro-4-methylphenyl)ethy l)glycyl)piperidine-4-carboxylic acid
  SITE 9: note = Amidated Tryptophan
YWNTFGLXW

SEQ ID NO: 109 - protein, 9 aa, synthetic construct
  SITE 1: note = D-Tyrosine
  SITE 2: note = D-Tryptophan
  SITE 6: note = Azaglycine
  SITE 8: note = 4-amino-1-((3,4-dimethylbenzyl)glycyl)piperidine-4- carboxylic acid
  SITE 9: note = Amidated Tryptophan
YWNTFGLXW

SEQ ID NO: 110 - protein, 9 aa, synthetic construct
  SITE 1: note = D-Tyrosine
  SITE 2: note = D-Tryptophan
  SITE 6: note = Azaglycine
  SITE 8: note = 4-amino-1-((4-chloro-3-fluorobenzyl)glycyl)piperidi ne-4-carboxylic acid
  SITE 9: note = Amidated Tryptophan
YWNTFGLXW

SEQ ID NO: 111 - protein, 9 aa, synthetic construct
  SITE 1: note = D-Tyrosine
  SITE 2: note = D-Tryptophan
  SITE 6: note = Azaglycine
  SITE 8: note = 4-amino-1-((4-carboxybenzyl)glycyl)piperidine-4-car boxylic acid
  SITE 9: note = Amidated Tryptophan
YWNTFGLXW

SEQ ID NO: 112 - protein, 9 aa, synthetic construct
  SITE 1: note = D-Tyrosine
  SITE 2: note = D-Tryptophan
  SITE 6: note = Azaglycine
  SITE 8: note = 4-amino-1-((4-carboxy-2,3-difluorobenzyl)glycyl)pip eridine-4-carboxylic acid
  SITE 9: note = Amidated Tryptophan
YWNTFGLXW

SEQ ID NO: 113 - protein, 9 aa, synthetic construct
  SITE 1: note = D-Tyrosine
  SITE 2: note = D-Tryptophan
  SITE 6: note = Azaglycine
  SITE 8: note = 4-amino-1-((3-fluoro-4-methoxybenzyl)glycyl)piperid ine-4-carboxylic acid
  SITE 9: note = Amidated Tryptophan
YWNTFGLXW

SEQ ID NO: 114 - protein, 9 aa, synthetic construct
  SITE 1: note = D-Tyrosine
  SITE 2: note = D-Tryptophan
  SITE 6: note = Azaglycine
  SITE 8: note = 4-amino-1-((3-chloro-4-methylbenzyl)glycyl)piperidi ne-4-carboxylic acid
  SITE 9: note = Amidated Tryptophan
YWNTFGLXW

SEQ ID NO: 115 - protein, 9 aa, synthetic construct
  SITE 1: note = D-Tyrosine
  SITE 2: note = D-Tryptophan
  SITE 6: note = Azaglycine
  SITE 8: note = 4-amino-1-((3,4,5-trifluorobenzyl)glycyl)piperidine -4-carboxylic acid
  SITE 9: note = Amidated Tryptophan
YWNTFGLXW

SEQ ID NO: 116 - protein, 9 aa, synthetic construct
  SITE 1: note = D-Tyrosine
  SITE 2: note = D-Tryptophan
  SITE 6: note = Azaglycine
  SITE 8: note = 4-amino-1-((4-(methylamino)benzyl)glycyl)piperidine -4-carboxylic acid
  SITE 9: note = Amidated Tryptophan
YWNTFGLXW

SEQ ID NO: 117 - protein, 9 aa, synthetic construct
  SITE 1: note = D-Tyrosine
  SITE 2: note = D-Tryptophan
  SITE 6: note = Azaglycine
  SITE 8: note = (Z)-4-amino-1-(N'-((3-fluoro-4-methylbenzyl)carbamo yl)carbamimidoyl)piperidine-4-carboxylic acid
  SITE 9: note = Amidated Tryptophan
YWNTFGLXW

SEQ ID NO: 118 - protein, 9 aa, synthetic construct
  SITE 1: note = D-Tyrosine
  SITE 2: note = D-Tryptophan
  SITE 6: note = Azaglycine
  SITE 8: note = 4-amino-1-((3-fluoro-4-methylbenzyl)glycyl)piperidi ne-4-carboxylic acid
  SITE 9: note = Amidated Tryptophan
YWNTFGLXW

SEQ ID NO: 119 - protein, 9 aa, synthetic construct
  SITE 1: note = D-Tyrosine
  SITE 2: note = D-Tryptophan
  SITE 6: note = Azaglycine
  SITE 8: note = 4-amino-1-(N-methylcarbamimidoyl)piperidine-4-carbo xylic acid
  SITE 9: note = Amidated Tryptophan
YWNTFGLXW

SEQ ID NO: 120 - protein, 5 aa, synthetic construct
  SITE 4: note = 4-amino-1-(N-methylcarbamimidoyl)piperidine-4-carbo xylic acid
  SITE 5: note = Amidated Tryptophan
FGLXW

SEQ ID NO: 121 - protein, 7 aa, synthetic construct
  SITE 1: note = D-Tyrosine
  SITE 2: note = hydroxyproline
  SITE 5: note = (S)-2-(4-((S)-1-amino-2-phenylethyl)-1H-1,2,3-triaz ol-1-yl)-4-methylpentanoic acid
  SITE 6: note = 4-amino-1-((3-fluoro-4-methylbenzyl)glycyl)piperidi ne-4-carboxamide
  SITE 7: note = Amidated Tryptophan
YXNTXXW

SEQ ID NO: 122 - protein, 7 aa, synthetic construct
  SITE 1: note = D-Asparagine
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
NGFGLXW

SEQ ID NO: 123 - protein, 7 aa, synthetic construct
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
NGFGLXW

SEQ ID NO: 124 - protein, 7 aa, synthetic construct
  SITE 1: note = D-Asparagine
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
NTFGLXW

SEQ ID NO: 125 - protein, 8 aa, synthetic construct
  SITE 1: note = D-Tryptophan
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
WNTFGLXW

SEQ ID NO: 126 - protein, 6 aa, synthetic construct
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
WFGLXW

SEQ ID NO: 127 - protein, 6 aa, synthetic construct
  SITE 1: note = (S)-2-amino-3-(pyridin-2-yl)propanoic acid
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 128 - protein, 9 aa, synthetic construct
  SITE 1: note = D-Tyrosine
  SITE 2: note = D-Tryptophan
  SITE 6: note = Azaglycine
  SITE 8: note = 4-amino-1-((3-fluoro-4-methylbenzyl)glycyl)piperidi ne-4-carboxamide
  SITE 9: note = Amidated 4-cyano phenylalanine
YWNTFGLXF

SEQ ID NO: 129 - protein, 9 aa, synthetic construct
  SITE 1: note = D-Tyrosine
  SITE 2: note = D-Tryptophan
  SITE 6: note = Azaglycine
  SITE 8: note = 4-amino-1-((3-fluoro-4-methylbenzyl)glycyl)piperidi ne-4-carboxamide
  SITE 9: note = Amidated 3-(4-pyridyl)alanine
YWNTFGLXX

SEQ ID NO: 130 - protein, 10 aa, synthetic construct
  SITE 1: note = D-Tyrosine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated 3-(4-pyridyl)alanine
YNWNTFGLXX

SEQ ID NO: 131 - protein, 10 aa, synthetic construct
  SITE 1: note = D-Tyrosine
  SITE 2: note = D-Histidine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated 3-(4-pyridyl)alanine
YHWNTFGLXX

SEQ ID NO: 132 - protein, 9 aa, synthetic construct
  SITE 1: note = D-Tyrosine
  SITE 2: note = Azaglycine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YGNTFGLXW

SEQ ID NO: 133 - protein, 9 aa, synthetic construct
  SITE 1: note = D-Tyrosine
  SITE 2: note = D-Tryptophan
  SITE 6: note = Azaglycine
  SITE 8: note = 4-amino-1-((3-fluoro-4-methylbenzyl)glycyl)piperidi ne-4-carboxamide
  SITE 9: note = Amidated Phenylalanine
YWNAFGLXF

SEQ ID NO: 134 - protein, 9 aa, synthetic construct
  SITE 1: note = D-Tyrosine
  SITE 2: note = D-Tryptophan
  SITE 6: note = Azaglycine
  SITE 8: note = 4-amino-1-((3-fluoro-4-methylbenzyl)glycyl)piperidi ne-4-carboxamide
  SITE 9: note = Amidated Leucine
YWNAFGLXL

SEQ ID NO: 135-140 - skipped (6 entries)

SEQ ID NO: 141 - protein, 9 aa, synthetic construct
  SITE 1: note = D-Tyrosine
  SITE 2: note = D-Tryptophan
  SITE 6: note = Azaglycine
  SITE 8: note = 4-amino-1-((4-methoxybenzyl)glycyl)piperidine-4-car boxylic acid
  SITE 9: note = Amidated Phenylalanine
YWNTFGLXF

SEQ ID NO: 142-145 - skipped (4 entries)

SEQ ID NO: 146 - protein, 10 aa, synthetic construct
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
GYWNTFGLXW

SEQ ID NO: 147 - protein, 7 aa, synthetic construct
  SITE 3: note = 3-Fluorophenylalanine
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
NTFGLXW

SEQ ID NO: 148 - protein, 8 aa, synthetic construct
  SITE 4: note = 3-Fluorophenylalanine
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
ENTFGLXW

SEQ ID NO: 149 - protein, 8 aa, synthetic construct
  SITE 1: note = D-Glutamic Acid
  SITE 4: note = 3-Fluorophenylalanine
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
ENTFGLXW

SEQ ID NO: 150 - protein, 8 aa, synthetic construct
  SITE 4: note = 3-Fluorophenylalanine
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
KNTFGLXW

SEQ ID NO: 151 - protein, 7 aa, synthetic construct
  SITE 3: note = 3-Fluorophenylalanine
  SITE 4: note = Azaglycine
  SITE 6: note = 4-amino-1-((3-fluoro-4-methylbenzyl)glycyl)piperidi ne-4-carboxamide
  SITE 7: note = Amidated 3-(4-pyridyl)alanine
NTFGLXX

SEQ ID NO: 152 - protein, 8 aa, synthetic construct
  SITE 4: note = 3-Fluorophenylalanine
  SITE 5: note = Azaglycine
  SITE 7: note = 4-amino-1-((3-fluoro-4-methylbenzyl)glycyl)piperidi ne-4-carboxamide
  SITE 8: note = Amidated 3-(4-pyridyl)alanine
ENTFGLXX

SEQ ID NO: 153 - protein, 5 aa, synthetic construct
  SITE 2: note = Azaglycine
  SITE 4: note = 4-amino-1-((3-fluoro-4-methylbenzyl)glycyl)piperidi ne-4-carboxamide
  SITE 5: note = Amidated Tyrosine
FGLXY

SEQ ID NO: 154 - protein, 9 aa, synthetic construct
  SITE 1: note = D-Tyrosine
  SITE 2: note = D-Tryptophan
  SITE 6: note = Azaglycine
  SITE 7: note = (S)-2-aminopentanoic acid
  SITE 8: note = 4-amino-1-((3-fluoro-4-methylbenzyl)glycyl)piperidi ne-4-carboxamide
  SITE 9: note = Amidated 3-(4-pyridyl)alanine
YWNTFGXXX

SEQ ID NO: 155 - protein, 9 aa, synthetic construct
  SITE 2: note = D-Tryptophan
  SITE 6: note = Azaglycine
  SITE 8: note = 4-amino-1-R1-piperidine-4-carboxylic acid
  SITE 9: note = Amidated Tryptophan
YWNTFGLXW

SEQ ID NO: 156 - protein, 9 aa, synthetic construct
  SITE 1: note = D-Tyrosine
  SITE 2: note = D-Tryptophan
  SITE 6: note = Azaglycine
  SITE 8: note = 4-amino-1-R1-piperidine-4-carboxylic acid
  SITE 9: note = Amidated Tryptophan
YWNTFGLXW

SEQ ID NO: 157 - protein, 9 aa, synthetic construct
  SITE 1: note = D-Tyrosine
  SITE 2: note = D-Tryptophan
  SITE 5: note = 3-Fluorophenylalanine
  SITE 6: note = Azaglycine
  SITE 8: note = 4-amino-1-R1-piperidine-4-carboxylic acid
  SITE 9: note = Amidated Tryptophan
YWNTFGLXW

SEQ ID NO: 158 - protein, 9 aa, synthetic construct
  SITE 2: note = D-Tryptophan
  SITE 5: note = 3-Fluorophenylalanine
  SITE 6: note = Azaglycine
  SITE 8: note = 4-amino-1-R1-piperidine-4-carboxylic acid
  SITE 9: note = Amidated Tryptophan
YWNTFGLXW

SEQ ID NO: 159 - protein, 5 aa, synthetic construct
  SITE 2: note = Azaglycine
  SITE 4: note = 4-amino-1-R1-piperidine-4-carboxylic acid
  SITE 5: note = Amidated Tryptophan
FGLXW

SEQ ID NO: 160 - protein, 5 aa, synthetic construct
  SITE 2: note = Azaglycine
  SITE 3: note = D-Leucine
  SITE 4: note = 4-amino-1-R1-piperidine-4-carboxylic acid
  SITE 5: note = Amidated Tryptophan
FGLXW

SEQ ID NO: 161 - protein, 5 aa, synthetic construct
  SITE 2: note = Azaglycine
  SITE 4: note = 4-amino-1-R1-piperidine-4-carboxylic acid
  SITE 5: note = Me-Trp-NH2
FGLXW

SEQ ID NO: 162 - protein, 5 aa, synthetic construct
  SITE 4: note = 4-amino-1-R1-piperidine-4-carboxylic acid
  SITE 5: note = Amidated Tryptophan
FGLXW

SEQ ID NO: 163 - protein, 5 aa, synthetic construct
  SITE 3: note = Methyl-leucine
  SITE 4: note = 4-amino-1-R1-piperidine-4-carboxylic acid
  SITE 5: note = Amidated Tryptophan
FGLXW

SEQ ID NO: 164 - protein, 5 aa, synthetic construct
  SITE 1: note = alpha-Me-Phenylalanine
  SITE 2: note = Azaglycine
  SITE 4: note = 4-amino-1-R1-piperidine-4-carboxylic acid
  SITE 5: note = Amidated Tryptophan
FGLXW

SEQ ID NO: 165 - protein, 5 aa, synthetic construct
  SITE 2: note = Sarcosine
  SITE 4: note = 4-amino-1-R1-piperidine-4-carboxylic acid
  SITE 5: note = Amidated Tryptophan
FGLXW

SEQ ID NO: 166 - protein, 5 aa, synthetic construct
  SITE 2: note = Sarcosine
  SITE 3: note = Methyl-leucine
  SITE 4: note = 4-amino-1-R1-piperidine-4-carboxylic acid
  SITE 5: note = Amidated Tryptophan
FGLXW

SEQ ID NO: 167 - protein, 10 aa, synthetic construct
  SITE 7: note = Azaglycine
  SITE 9: note = 4-amino-1-R1-piperidine-4-carboxylic acid
  SITE 10: note = Amidated Tryptophan
AAAAAFGLXW

SEQ ID NO: 168 - protein, 10 aa, synthetic construct
  SITE 7: note = Azaglycine
  SITE 9: note = 4-amino-1-R1-piperidine-4-carboxylic acid
  SITE 10: note = Amidated Tryptophan
NNNNNFGLXW

SEQ ID NO: 169 - protein, 10 aa, synthetic construct
  SITE 7: note = Azaglycine
  SITE 9: note = 4-amino-1-R1-piperidine-4-carboxylic acid
  SITE 10: note = Amidated Tryptophan
NNEENFGLXW

SEQ ID NO: 170 - protein, 10 aa, synthetic construct
  SITE 7: note = Azaglycine
  SITE 9: note = 4-amino-1-R1-piperidine-4-carboxylic acid
  SITE 10: note = Amidated Tryptophan
NNKENFGLXW

SEQ ID NO: 171 - protein, 7 aa, synthetic construct
  SITE 1: note = Sarcosine
  SITE 2: note = Sarcosine
  SITE 4: note = Azaglycine
  SITE 6: note = 4-amino-1-R1-piperidine-4-carboxylic acid
  SITE 7: note = Amidated Tryptophan
GGFGLXW

SEQ ID NO: 172 - protein, 7 aa, synthetic construct
  SITE 4: note = Azaglycine
  SITE 6: note = 4-amino-1-R1-piperidine-4-carboxylic acid
  SITE 7: note = Amidated Tryptophan
NNFGLXW

SEQ ID NO: 173 - protein, 7 aa, synthetic construct
  SITE 4: note = Azaglycine
  SITE 6: note = 4-amino-1-R1-piperidine-4-carboxylic acid
  SITE 7: note = Amidated Tryptophan
KNFGLXW

SEQ ID NO: 174 - protein, 8 aa, synthetic construct
  SITE 4: note = 3-Fluorophenylalanine
  SITE 5: note = Azaglycine
  SITE 7: note = 4-amino-1-R1-piperidine-4-carboxylic acid
  SITE 8: note = Amidated Tryptophan
KNTFGLXW

SEQ ID NO: 175 - protein, 9 aa, synthetic construct
  SITE 6: note = Azaglycine
  SITE 8: note = 4-amino-1-R1-piperidine-4-carboxylic acid
  SITE 9: note = Amidated Tryptophan
YPNTFGLXW

SEQ ID NO: 176 - protein, 6 aa, synthetic construct
  SITE 3: note = Azaglycine
  SITE 5: note = 4-amino-1-R1-piperidine-4-carboxylic acid
  SITE 6: note = Amidated Tryptophan
NFGLXW

SEQ ID NO: 177 - protein, 9 aa, synthetic construct
  SITE 2: note = 3-(4-pyridyl)alanine
  SITE 6: note = Azaglycine
  SITE 8: note = 4-amino-1-R1-piperidine-4-carboxylic acid
  SITE 9: note = Amidated Tryptophan
YXNTFGLXW

SEQ ID NO: 178 - protein, 9 aa, synthetic construct
  SITE 1: note = D-Tyrosine
  SITE 2: note = 3-(4-pyridyl)alanine
  SITE 6: note = Azaglycine
  SITE 8: note = 4-amino-1-R1-piperidine-4-carboxylic acid
  SITE 9: note = Amidated Tryptophan
YXNTFGLXW

SEQ ID NO: 179 - protein, 9 aa, synthetic construct
  SITE 1: note = D-Tyrosine
  SITE 2: note = D-3-(4-pyridyl)alanine
  SITE 6: note = Azaglycine
  SITE 8: note = 4-amino-1-R1-piperidine-4-carboxylic acid
  SITE 9: note = Amidated Tryptophan
YXNTFGLXW

SEQ ID NO: 180 - protein, 8 aa, synthetic construct
  SITE 1: note = D-3-(4-pyridyl)alanine
  SITE 5: note = Azaglycine
  SITE 7: note = 4-amino-1-R1-piperidine-4-carboxylic acid
  SITE 8: note = Amidated Tryptophan
XNTFGLXW

SEQ ID NO: 181 - protein, 9 aa, synthetic construct
  SITE 1: note = D-Tyrosine
  SITE 5: note = 3-Fluorophenylalanine
  SITE 6: note = Azaglycine
  SITE 8: note = 4-amino-1-R1-piperidine-4-carboxylic acid
  SITE 9: note = Amidated Tryptophan
YENTFGLXW

SEQ ID NO: 182 - protein, 9 aa, synthetic construct
  SITE 5: note = 3-Fluorophenylalanine
  SITE 6: note = Azaglycine
  SITE 8: note = 4-amino-1-R1-piperidine-4-carboxylic acid
  SITE 9: note = Amidated Tryptophan
YENTFGLXW

SEQ ID NO: 183 - protein, 7 aa, synthetic construct
  SITE 1: note = D-Asparagine
  SITE 4: note = Azaglycine
  SITE 6: note = 4-amino-1-R1-piperidine-4-carboxylic acid
  SITE 7: note = Amidated Tryptophan
NTFGLXW

SEQ ID NO: 184 - protein, 7 aa, synthetic construct
  SITE 4: note = Azaglycine
  SITE 6: note = 4-amino-1-R1-piperidine-4-carboxylic acid
  SITE 7: note = Amidated Tryptophan
NTFGLXW

SEQ ID NO: 185 - protein, 7 aa, synthetic construct
  SITE 3: note = 3-Fluorophenylalanine
  SITE 4: note = Azaglycine
  SITE 6: note = 4-amino-1-R1-piperidine-4-carboxylic acid
  SITE 7: note = Amidated Tryptophan
NTFGLXW

SEQ ID NO: 186 - protein, 9 aa, synthetic construct
  SITE 5: note = 3-Fluorophenylalanine
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 187 - protein, 8 aa, synthetic construct
  SITE 1: note = D-Tryptophan
  SITE 4: note = 3-Fluorophenylalanine
  SITE 5: note = Azaglycine
  SITE 7: note = 4-amino-1-R1-piperidine-4-carboxylic acid
  SITE 8: note = Amidated Tryptophan
WNTFGLXW

SEQ ID NO: 188 - protein, 8 aa, synthetic construct
  SITE 5: note = Azaglycine
  SITE 7: note = 4-amino-1-R1-piperidine-4-carboxylic acid
  SITE 8: note = Amidated Tryptophan
WNTFGLXW

SEQ ID NO: 189 - protein, 10 aa, synthetic construct
  SITE 7: note = Azaglycine
  SITE 9: note = 4-amino-1-R1-piperidine-4-carboxylic acid
  SITE 10: note = Amidated Tryptophan
YHWNTFGLXW

SEQ ID NO: 190 - protein, 10 aa, synthetic construct
  SITE 1: note = D-Tyrosine
  SITE 7: note = Azaglycine
  SITE 9: note = 4-amino-1-R1-piperidine-4-carboxylic acid
  SITE 10: note = Amidated Tryptophan
YHWNTFGLXW

SEQ ID NO: 191 - protein, 9 aa, synthetic construct
  SITE 1: note = Sarcosine
  SITE 2: note = Sarcosine
  SITE 3: note = Sarcosine
  SITE 4: note = Sarcosine
  SITE 8: note = 4-amino-1-R1-piperidine-4-carboxylic acid
  SITE 9: note = Amidated Tryptophan
GGGGFGLXW

SEQ ID NO: 192 - protein, 5 aa, synthetic construct
  SITE 2: note = Azaglycine
  SITE 4: note = 4-amino-1-R1-piperidine-4-carboxylic acid
  SITE 5: note = Amidated Tryptophan
YGLXW

SEQ ID NO: 193 - skipped

SEQ ID NO: 194 - protein, 8 aa, synthetic construct
  SITE 1: note = Sarcosine
  SITE 2: note = Sarcosine
  SITE 3: note = Sarcosine
  SITE 4: note = Sarcosine
  SITE 5: note = Sarcosine
  SITE 7: note = 4-amino-1-R1-piperidine-4-carboxylic acid
  SITE 8: note = Amidated Tryptophan
GGGGGFXW

SEQ ID NO: 195 - protein, 8 aa, synthetic construct
  SITE 1: note = Sarcosine
  SITE 2: note = Sarcosine
  SITE 3: note = Sarcosine
  SITE 4: note = Sarcosine
  SITE 5: note = Sarcosine
  SITE 6: note = phenylglycine
  SITE 7: note = 4-amino-1-R1-piperidine-4-carboxylic acid
  SITE 8: note = Amidated Tryptophan
GGGGGGXW

SEQ ID NO: 196 - skipped

SEQ ID NO: 197 - protein, 5 aa, synthetic construct
  SITE 2: note = Azaglycine
  SITE 4: note = 4-amino-1-R1-piperidine-4-carboxylic acid
  SITE 5: note = Amidated Phenylalanine
FGLXF

SEQ ID NO: 198 - protein, 9 aa, synthetic construct
  SITE 2: note = D-Tryptophan
  SITE 5: note = 3-Fluorophenylalanine
  SITE 6: note = Azaglycine
  SITE 8: note = 4-amino-1-R1-piperidine-4-carboxylic acid
  SITE 9: note = Amidated Phenylalanine
YWNTFGLXF

SEQ ID NO: 199 - protein, 9 aa, synthetic construct
  SITE 1: note = D-Tyrosine
  SITE 2: note = D-Tryptophan
  SITE 6: note = Azaglycine
  SITE 8: note = 4-amino-1-R1-piperidine-4-carboxylic acid
  SITE 9: note = Amidated Phenylalanine
YWNTFGLXF

SEQ ID NO: 200 - protein, 9 aa, synthetic construct
  SITE 6: note = Azaglycine
  SITE 8: note = 4-amino-1-R1-piperidine-4-carboxylic acid
  SITE 9: note = Amidated 4-cyano phenylalanine
YWNTFGLXF

SEQ ID NO: 201 - protein, 8 aa, synthetic construct
  SITE 5: note = Azaglycine
  SITE 7: note = 4-amino-1-R1-piperidine-4-carboxylic acid
  SITE 8: note = Amidated Phenylalanine
ENTFGLXF

SEQ ID NO: 202 - protein, 8 aa, synthetic construct
  SITE 5: note = Azaglycine
  SITE 7: note = 4-amino-1-R1-piperidine-4-carboxylic acid
  SITE 8: note = Amidated 4-cyano phenylalanine
ENTFGLXF

SEQ ID NO: 203 - protein, 8 aa, synthetic construct
  SITE 1: note = D-Glutamic Acid
  SITE 5: note = Azaglycine
  SITE 7: note = 4-amino-1-R1-piperidine-4-carboxylic acid
  SITE 8: note = Amidated 4-cyano phenylalanine
ENTFGLXF

SEQ ID NO: 204 - protein, 7 aa, synthetic construct
  SITE 4: note = Azaglycine
  SITE 6: note = 4-amino-1-R1-piperidine-4-carboxylic acid
  SITE 7: note = Amidated Phenylalanine
NTFGLXF

SEQ ID NO: 205 - protein, 7 aa, synthetic construct
  SITE 4: note = Azaglycine
  SITE 6: note = 4-amino-1-R1-piperidine-4-carboxylic acid
  SITE 7: note = Amidated 4-cyano phenylalanine
NTFGLXF

SEQ ID NO: 206 - protein, 5 aa, synthetic construct
  SITE 2: note = Azaglycine
  SITE 4: note = 4-amino-1-R1-piperidine-4-carboxylic acid
  SITE 5: note = Amidated 4-cyano phenylalanine
FGLXF

SEQ ID NO: 207 - protein, 9 aa, synthetic construct
  SITE 1: note = D-Tyrosine
  SITE 6: note = Azaglycine
  SITE 8: note = 4-amino-1-R1-piperidine-4-carboxylic acid
  SITE 9: note = Amidated Phenylalanine
YWNAFGLXF

SEQ ID NO: 208 - protein, 9 aa, synthetic construct
  SITE 2: note = D-3-(2-naphthyl)-alanine
  SITE 6: note = Azaglycine
  SITE 8: note = 4-amino-1-R1-piperidine-4-carboxylic acid
  SITE 9: note = Amidated Phenylalanine
YXNTFGLXF

SEQ ID NO: 209 - protein, 9 aa, synthetic construct
  SITE 1: note = D-Tyrosine
  SITE 2: note = D-3-(2-naphthyl)-alanine
  SITE 6: note = Azaglycine
  SITE 8: note = 4-amino-1-R1-piperidine-4-carboxylic acid
  SITE 9: note = Amidated Phenylalanine
YXNTFGLXF

SEQ ID NO: 210 - protein, 9 aa, synthetic construct
  SITE 1: note = D-Tyrosine
  SITE 2: note = D-3-(2-naphthyl)-alanine
  SITE 6: note = Azaglycine
  SITE 8: note = 4-amino-1-R1-piperidine-4-carboxylic acid
  SITE 9: note = Amidated Phenylalanine
YXNTFGLXF

SEQ ID NO: 211 - protein, 8 aa, synthetic construct
  SITE 1: note = 3-(2-naphthyl)alanine
  SITE 5: note = Azaglycine
  SITE 7: note = 4-amino-1-R1-piperidine-4-carboxylic acid
  SITE 8: note = Amidated Phenylalanine
XNTFGLXF

SEQ ID NO: 212 - protein, 9 aa, synthetic construct
  SITE 1: note = D-Tyrosine
  SITE 2: note = 3-(2-naphthyl)alanine
  SITE 6: note = Azaglycine
  SITE 8: note = 4-amino-1-R1-piperidine-4-carboxylic acid
  SITE 9: note = Amidated 4-cyano phenylalanine
YXNTFGLXF

SEQ ID NO: 213 - protein, 8 aa, synthetic construct
  SITE 7: note = 4-amino-1-R1-piperidine-4-carboxylic acid
  SITE 8: note = Amidated Phenylalanine
TNRNFLXF

SEQ ID NO: 214 - protein, 9 aa, synthetic construct
  SITE 1: note = D-Tyrosine
  SITE 2: note = 3-(2-naphthyl)alanine
  SITE 6: note = Azaglycine
  SITE 8: note = 4-amino-1-R1-piperidine-4-carboxylic acid
  SITE 9: note = Amidated Tryptophan
YXNTFGLXW

SEQ ID NO: 215 - protein, 9 aa, synthetic construct
  SITE 1: note = D-Tyrosine
  SITE 2: note = D-3-(2-naphthyl)-alanine
  SITE 6: note = Azaglycine
  SITE 8: note = 4-amino-1-R1-piperidine-4-carboxylic acid
  SITE 9: note = Amidated Tryptophan
YXNTFGLXW

SEQ ID NO: 216 - protein, 9 aa, synthetic construct
  SITE 1: note = D-Tyrosine
  SITE 6: note = Azaglycine
  SITE 8: note = 4-amino-1-R1-piperidine-4-carboxylic acid
  SITE 9: note = Amidated Tryptophan
YRNTFGLXW

SEQ ID NO: 217 - protein, 10 aa, synthetic construct
  SITE 1: note = D-Tyrosine
  SITE 7: note = Azaglycine
  SITE 9: note = 4-amino-1-R1-piperidine-4-carboxylic acid
  SITE 10: note = Amidated Tryptophan
YNRNTFGLXW

SEQ ID NO: 218 - protein, 10 aa, synthetic construct
  SITE 1: note = Sarcosine
  SITE 2: note = Sarcosine
  SITE 3: note = Sarcosine
  SITE 4: note = Sarcosine
  SITE 5: note = Sarcosine
  SITE 7: note = Azaglycine
  SITE 9: note = 4-amino-1-R1-piperidine-4-carboxylic acid
  SITE 10: note = Amidated Tryptophan
GGGGGFGLXW

SEQ ID NO: 219 - protein, 7 aa, synthetic construct
  SITE 4: note = Azaglycine
  SITE 6: note = 4-amino-1-R1-piperidine-4-carboxylic acid
  SITE 7: note = Amidated Tryptophan
ENFGLXW

SEQ ID NO: 220 - protein, 8 aa, synthetic construct
  SITE 5: note = Azaglycine
  SITE 7: note = 4-amino-1-R1-piperidine-4-carboxylic acid
  SITE 8: note = Amidated Tryptophan
ENTFGLXW

SEQ ID NO: 221 - protein, 8 aa, synthetic construct
  SITE 4: note = 3-Fluorophenylalanine
  SITE 5: note = Azaglycine
  SITE 7: note = 4-amino-1-R1-piperidine-4-carboxylic acid
  SITE 8: note = Amidated Tryptophan
ENTFGLXW

SEQ ID NO: 222 - protein, 8 aa, synthetic construct
  SITE 1: note = D-Glutamic Acid
  SITE 4: note = 3-Fluorophenylalanine
  SITE 5: note = Azaglycine
  SITE 7: note = 4-amino-1-R1-piperidine-4-carboxylic acid
  SITE 8: note = Amidated Tryptophan
ENTFGLXW

SEQ ID NO: 223 - protein, 9 aa, synthetic construct
  SITE 1: note = D-Tyrosine
  SITE 6: note = Azaglycine
  SITE 8: note = 4-amino-1-R1-piperidine-4-carboxylic acid
  SITE 9: note = Amidated Tryptophan
YANTFGLXW

SEQ ID NO: 224 - protein, 9 aa, synthetic construct
  SITE 1: note = D-Tyrosine
  SITE 6: note = Azaglycine
  SITE 8: note = 4-amino-1-R1-piperidine-4-carboxylic acid
  SITE 9: note = Amidated Tryptophan
YPNTFGLXW

SEQ ID NO: 225 - protein, 7 aa, synthetic construct
  SITE 1: note = D-Asparagine
  SITE 4: note = Azaglycine
  SITE 6: note = 4-amino-1-R1-piperidine-4-carboxylic acid
  SITE 7: note = Amidated Tryptophan
NGFGLXW

SEQ ID NO: 226 - protein, 7 aa, synthetic construct
  SITE 4: note = Azaglycine
  SITE 6: note = 4-amino-1-R1-piperidine-4-carboxylic acid
  SITE 7: note = Amidated Tryptophan
NGFGLXW

SEQ ID NO: 227 - protein, 6 aa, synthetic construct
  SITE 3: note = Azaglycine
  SITE 5: note = 4-amino-1-R1-piperidine-4-carboxylic acid
  SITE 6: note = Amidated Tryptophan
WFGLXW

SEQ ID NO: 228 - protein, 6 aa, synthetic construct
  SITE 1: note = 2-Pal
  SITE 3: note = Azaglycine
  SITE 5: note = 4-amino-1-R1-piperidine-4-carboxylic acid
  SITE 6: note = Amidated Tryptophan
XFGLXW

SEQ ID NO: 229 - protein, 9 aa, synthetic construct
  SITE 2: note = Azaglycine
  SITE 6: note = Azaglycine
  SITE 8: note = 4-amino-1-R1-piperidine-4-carboxylic acid
  SITE 9: note = Amidated Tryptophan
YGNTFGLXW

SEQ ID NO: 230 - protein, 9 aa, synthetic construct
  SITE 1: note = D-Tyrosine
  SITE 2: note = D-Tryptophan
  SITE 6: note = Azaglycine
  SITE 8: note = 4-amino-1-R1-piperidine-4-carboxylic acid
  SITE 9: note = Amidated Leucine
YWNAFGLXL

SEQ ID NO: 231 - protein, 9 aa, synthetic construct
  SITE 1: note = D-Tyrosine
  SITE 2: note = D-Tryptophan
  SITE 6: note = Azaglycine
  SITE 8: note = 4-amino-1-R1-piperidine-4-carboxylic acid
  SITE 9: note = Amidated 3-(4-pyridyl)alanine
YWNTFGLXX

SEQ ID NO: 232 - protein, 10 aa, synthetic construct
  SITE 1: note = D-Tyrosine
  SITE 3: note = D-Tryptophan
  SITE 7: note = Azaglycine
  SITE 9: note = 4-amino-1-R1-piperidine-4-carboxylic acid
  SITE 10: note = Amidated 3-(4-pyridyl)alanine
YNWNTFGLXX

SEQ ID NO: 233 - protein, 10 aa, synthetic construct
  SITE 1: note = D-Tyrosine
  SITE 3: note = D-Tryptophan
  SITE 7: note = Azaglycine
  SITE 9: note = 4-amino-1-R1-piperidine-4-carboxylic acid
  SITE 10: note = Amidated 3-(4-pyridyl)alanine
YHWNTFGLXX

SEQ ID NO: 234 - protein, 9 aa, synthetic construct
  SITE 1: note = D-Tyrosine
  SITE 2: note = 4-iodophenylalanine
  SITE 5: note = 4-iodophenylalanine
  SITE 6: note = Azaglycine
  SITE 8: note = 4-amino-1-R1-piperidine-4-carboxylic acid
  SITE 9: note = Amidated 3-(4-pyridyl)alanine
YFNTFGLXX

SEQ ID NO: 235 - protein, 10 aa, synthetic construct
  SITE 1: note = D-Tyrosine
  SITE 3: note = D-Tryptophan
  SITE 7: note = Azaglycine
  SITE 9: note = 4-amino-1-R1-piperidine-4-carboxylic acid
  SITE 10: note = Amidated 3-(4-pyridyl)alanine
YNWNTFGLXX

SEQ ID NO: 236 - protein, 9 aa, synthetic construct
  SITE 1: note = D-Tyrosine
  SITE 2: note = (S)-2-amino-3-(anthracen-9-yl)propanoic acid
  SITE 6: note = Azaglycine
  SITE 7: note = Norleucine
  SITE 8: note = 4-amino-1-R1-piperidine-4-carboxylic acid
  SITE 9: note = Amidated 3-(4-pyridyl)alanine
YXNTFGXXX

SEQ ID NO: 237 - protein, 9 aa, synthetic construct
  SITE 1: note = D-Tyrosine
  SITE 2: note = (S)-2-amino-3-(anthracen-9-yl)propanoic acid
  SITE 6: note = Azaglycine
  SITE 8: note = 4-amino-1-R1-piperidine-4-carboxylic acid
  SITE 9: note = Amidated 3-(4-pyridyl)alanine
YXNTFGLXX

SEQ ID NO: 238 - protein, 8 aa, synthetic construct
  SITE 1: note = D-Tyrosine
  SITE 5: note = Azaglycine
  SITE 7: note = 4-amino-1-R1-piperidine-4-carboxylic acid
  SITE 8: note = Amidated 3-(4-pyridyl)alanine
YNTFGLXX

SEQ ID NO: 239 - protein, 8 aa, synthetic construct
  SITE 1: note = 3-(9-anthryl)-alanine
  SITE 5: note = Azaglycine
  SITE 7: note = 4-amino-1-R1-piperidine-4-carboxylic acid
  SITE 8: note = Amidated 3-(4-pyridyl)alanine
XNTFGLXX

SEQ ID NO: 240 - protein, 9 aa, synthetic construct
  SITE 1: note = D-Tyrosine
  SITE 2: note = 3-(9-anthryl)-alanine
  SITE 5: note = 4-iodophenylalanine
  SITE 6: note = Azaglycine
  SITE 8: note = 4-amino-1-R1-piperidine-4-carboxylic acid
  SITE 9: note = Amidated 3-(4-pyridyl)alanine
YXNTFGLXX

SEQ ID NO: 241 - protein, 7 aa, synthetic construct
  SITE 4: note = Azaglycine
  SITE 6: note = 4-amino-1-R1-piperidine-4-carboxylic acid
  SITE 7: note = Amidated 3-(4-pyridyl)alanine
NTFGLXX

SEQ ID NO: 242 - protein, 7 aa, synthetic construct
  SITE 3: note = 3-Fluorophenylalanine
  SITE 4: note = Azaglycine
  SITE 6: note = 4-amino-1-R1-piperidine-4-carboxylic acid
  SITE 7: note = Amidated 3-(4-pyridyl)alanine
NTFGLXX

SEQ ID NO: 243 - protein, 8 aa, synthetic construct
  SITE 4: note = 3-Fluorophenylalanine
  SITE 5: note = Azaglycine
  SITE 7: note = 4-amino-1-R1-piperidine-4-carboxylic acid
  SITE 8: note = Amidated 3-(4-pyridyl)alanine
ENTFGLXX

SEQ ID NO: 244 - protein, 9 aa, synthetic construct
  SITE 6: note = Azaglycine
  SITE 7: note = Nva
  SITE 8: note = 4-amino-1-R1-piperidine-4-carboxylic acid
  SITE 9: note = Amidated 3-(4-pyridyl)alanine
YWNTFGXXX

SEQ ID NO: 245 - protein, 9 aa, synthetic construct
  SITE 1: note = D-Tyrosine
  SITE 2: note = D-Tryptophan
  SITE 6: note = Azaglycine
  SITE 7: note = Nva
  SITE 8: note = 4-amino-1-R1-piperidine-4-carboxylic acid
  SITE 9: note = Amidated 3-(4-pyridyl)alanine
YWNTFGXXX

SEQ ID NO: 246 - protein, 9 aa, synthetic construct
  SITE 2: note = 3-(2-naphthyl)alanine
  SITE 6: note = Azaglycine
  SITE 8: note = 4-amino-1-R1-piperidine-4-carboxylic acid
  SITE 9: note = Amidated 3-(4-pyridyl)alanine
YXNTFGLXX

SEQ ID NO: 247 - protein, 9 aa, synthetic construct
  SITE 1: note = D-Tyrosine
  SITE 2: note = 3-(2-naphthyl)alanine
  SITE 6: note = Azaglycine
  SITE 8: note = 4-amino-1-R1-piperidine-4-carboxylic acid
  SITE 9: note = Amidated 3-(4-pyridyl)alanine
YXNTFGLXX

SEQ ID NO: 248 - protein, 8 aa, synthetic construct
  SITE 1: note = 3-(2-naphthyl)alanine
  SITE 5: note = Azaglycine
  SITE 7: note = 4-amino-1-R1-piperidine-4-carboxylic acid
  SITE 8: note = Amidated 3-(4-pyridyl)alanine
XNTFGLXX

SEQ ID NO: 249 - protein, 9 aa, synthetic construct
  SITE 1: note = D-Tyrosine
  SITE 2: note = 3-(4-pyridyl)alanine
  SITE 6: note = Azaglycine
  SITE 8: note = 4-amino-1-R1-piperidine-4-carboxylic acid
  SITE 9: note = Amidated 3-(4-pyridyl)alanine
YXNTFGLXX

SEQ ID NO: 250 - protein, 9 aa, synthetic construct
  SITE 1: note = D-Tyrosine
  SITE 2: note = D-3-(4-pyridyl)alanine
  SITE 6: note = Azaglycine
  SITE 8: note = 4-amino-1-R1-piperidine-4-carboxylic acid
  SITE 9: note = Amidated 3-(4-pyridyl)alanine
YXNTFGLXX

SEQ ID NO: 251 - protein, 8 aa, synthetic construct
  SITE 1: note = D-3-(4-pyridyl)alanine
  SITE 5: note = Azaglycine
  SITE 7: note = 4-amino-1-R1-piperidine-4-carboxylic acid
  SITE 8: note = Amidated 3-(4-pyridyl)alanine
XNTFGLXX

SEQ ID NO: 252 - protein, 8 aa, synthetic construct
  SITE 1: note = bGlu
  SITE 4: note = 3-Fluorophenylalanine
  SITE 5: note = Azaglycine
  SITE 7: note = 4-amino-1-R1-piperidine-4-carboxylic acid
  SITE 8: note = Amidated 3-(4-pyridyl)alanine
ENTFGLXX

SEQ ID NO: 253 - protein, 7 aa, synthetic construct
  SITE 3: note = 3-(4-pyridyl)alanine
  SITE 4: note = Azaglycine
  SITE 6: note = 4-amino-1-R1-piperidine-4-carboxylic acid
  SITE 7: note = Amidated 3-(4-pyridyl)alanine
NTXGLXX

SEQ ID NO: 254-256 - skipped (3 entries)

SEQ ID NO: 257 - protein, 10 aa, synthetic construct
  SITE 3: note = D-Tryptophan
  SITE 7: note = Azaglycine
  SITE 9: note = 4-amino-1-R1-piperidine-4-carboxylic acid
  SITE 10: note = Amidated Tryptophan
GYWNTFGLXW

SEQ ID NO: 258 - protein, 9 aa, synthetic construct
  SITE 1: note = D-Tyrosine
  SITE 2: note = 4-benzoylphenylalanine
  SITE 6: note = Azaglycine
  SITE 8: note = 4-amino-1-R1-piperidine-4-carboxylic acid
  SITE 9: note = Amidated Tryptophan
YXNTFGLXW

SEQ ID NO: 259 - protein, 9 aa, synthetic construct
  SITE 1: note = D-Tyrosine
  SITE 2: note = 3-(9-anthryl)-alanine
  SITE 6: note = Azaglycine
  SITE 8: note = 4-amino-1-R1-piperidine-4-carboxylic acid
  SITE 9: note = Amidated Tryptophan
YXNTFGLXW

SEQ ID NO: 260 - skipped

SEQ ID NO: 261 - protein, 5 aa, synthetic construct
  SITE 4: note = 4-amino-1-R1-piperidine-4-carboxylic acid
  SITE 5: note = Amidated Tyrosine
FGLXY

SEQ ID NO: 262 - protein, 5 aa, synthetic construct
  SITE 2: note = Azaglycine
  SITE 4: note = 4-amino-1-R1-piperidine-4-carboxylic acid
  SITE 5: note = Amidated Tyrosine
FGLXY

SEQ ID NO: 263 - protein, 6 aa, synthetic construct
  SITE 3: note = Azaglycine
  SITE 5: note = 4-amino-1-R1-piperidine-4-carboxylic acid
  SITE 6: note = Amidated Tyrosine
TFGLXY

SEQ ID NO: 264 - protein, 7 aa, synthetic construct
  SITE 4: note = Azaglycine
  SITE 6: note = 4-amino-1-R1-piperidine-4-carboxylic acid
  SITE 7: note = Amidated Tyrosine
NTFGLXY

SEQ ID NO: 265 - protein, 8 aa, synthetic construct
  SITE 1: note = D-Glutamic Acid
  SITE 5: note = Azaglycine
  SITE 7: note = 4-amino-1-R1-piperidine-4-carboxylic acid
  SITE 8: note = Amidated Tyrosine
ENTFGLXY

SEQ ID NO: 266 - protein, 9 aa, synthetic construct
  SITE 1: note = D-Tyrosine
  SITE 2: note = 3-(2-naphthyl)alanine
  SITE 6: note = Azaglycine
  SITE 8: note = 4-amino-1-R1-piperidine-4-carboxylic acid
  SITE 9: note = Amidated Tyrosine
YXNTFGLXY

SEQ ID NO: 267 - protein, 9 aa, synthetic construct
  SITE 1: note = D-Tyrosine
  SITE 2: note = D-3-(2-naphthyl)-alanine
  SITE 6: note = Azaglycine
  SITE 8: note = 4-amino-1-R1-piperidine-4-carboxylic acid
  SITE 9: note = Amidated Tyrosine
YXNTFGLXY

SEQ ID NO: 268 - protein, 10 aa, synthetic construct
  SITE 2: note = D-Tyrosine
  SITE 3: note = D-3-(4-pyridyl)alanine
  SITE 7: note = Azaglycine
  SITE 9: note = 4-amino-1-R1-piperidine-4-carboxylic acid
  SITE 10: note = Amidated Tyrosine
GYXNTFGLXY

SEQ ID NO: 269 - protein, 9 aa, synthetic construct
  SITE 1: note = D-Tyrosine
  SITE 2: note = D-Tryptophan
  SITE 6: note = Azaglycine
  SITE 8: note = 4-amino-1-R1-piperidine-4-carboxylic acid
  SITE 9: note = Amidated Tyrosine
YWNTFGLXY

SEQ ID NO: 270 - protein, 9 aa, synthetic construct
  SITE 1: note = D-Tyrosine
  SITE 2: note = Bip
  SITE 6: note = Azaglycine
  SITE 8: note = 4-amino-1-R1-piperidine-4-carboxylic acid
  SITE 9: note = Amidated Tryptophan
YXNTFGLXW

SEQ ID NO: 271 - protein, 9 aa, synthetic construct
  SITE 1: note = D-Tyrosine
  SITE 2: note = Bip
  SITE 6: note = Azaglycine
  SITE 8: note = 4-amino-1-R1-piperidine-4-carboxylic acid
  SITE 9: note = Amidated D-Tryptophan
YXNTFGLXW

SEQ ID NO: 272 - protein, 8 aa, synthetic construct
  SITE 4: note = beta-(2-thienyl)-Ala
  SITE 5: note = Azaglycine
  SITE 7: note = 4-amino-1-R1-piperidine-4-carboxylic acid
  SITE 8: note = Amidated Tryptophan
ENTAGLXW

SEQ ID NO: 273 - protein, 9 aa, synthetic construct
  SITE 1: note = 4-iodophenylalanine
  SITE 6: note = Azaglycine
  SITE 8: note = 4-amino-1-R1-piperidine-4-carboxylic acid
  SITE 9: note = Amidated Tryptophan
FWNTFGLXW

SEQ ID NO: 274 - protein, 9 aa, synthetic construct
  SITE 6: note = Azaglycine
  SITE 8: note = 4-amino-1-R1-piperidine-4-carboxylic acid
  SITE 9: note = Amidated Tryptophan
KWNTFGLXW

SEQ ID NO: 275 - protein, 6 aa, synthetic construct
  SITE 3: note = Azaglycine
  SITE 5: note = 4-amino-1-R1-piperidine-4-carboxylic acid
  SITE 6: note = Amidated Tryptophan
TFGLXW

SEQ ID NO: 276 - protein, 7 aa, synthetic construct
  SITE 4: note = Azaglycine
  SITE 6: note = 4-amino-1-R1-piperidine-4-carboxylic acid
  SITE 7: note = Amidated Tryptophan
ETFGLXW

SEQ ID NO: 277 - protein, 5 aa, synthetic construct
  SITE 2: note = Azaglycine
  SITE 4: note = 4-amino-1-R1-piperidine-4-carboxylic acid
  SITE 5: note = Amidated Histidine
FGLXH

SEQ ID NO: 278 - protein, 7 aa, synthetic construct
  SITE 4: note = Azaglycine
  SITE 6: note = 4-amino-1-R1-piperidine-4-carboxylic acid
  SITE 7: note = Tyr(3-NO2)-NH2
NTFGLXY

SEQ ID NO: 279 - protein, 9 aa, synthetic construct
  SITE 1: note = D-Tyrosine
  SITE 2: note = D-Tryptophan
  SITE 5: note = biphenylalanine
  SITE 6: note = Azaglycine
  SITE 8: note = 4-amino-1-R1-piperidine-4-carboxylic acid
  SITE 9: note = Amidated Tryptophan
YWNTXGLXW

SEQ ID NO: 280 - protein, 8 aa, synthetic construct
  SITE 1: note = 3-(2-(2-hydroxyethoxy)ethoxy)propanoic acid D-Lysine bound to an optional linker and chelating moiety or a radionuclide complex
  SITE 5: note = Azaglycine
  SITE 7: note = 4-amino-1-R1-piperidine-4-carboxylic acid
  SITE 8: note = Amidated 3-(4-pyridyl)alanine
KNTFGLXX

SEQ ID NO: 281 - protein, 9 aa, synthetic construct
  SITE 2: note = hydroxyproline
  SITE 6: note = Azaglycine
  SITE 8: note = 4-amino-1-R1-piperidine-4-carboxylic acid
  SITE 9: note = Amidated Tryptophan
YXNTFGLXW

SEQ ID NO: 282 - protein, 9 aa, synthetic construct
  SITE 1: note = D-Tyrosine
  SITE 2: note = hydroxyproline
  SITE 6: note = Azaglycine
  SITE 8: note = 4-amino-1-R1-piperidine-4-carboxylic acid
  SITE 9: note = Amidated Tryptophan
YXNTFGLXW

SEQ ID NO: 283 - protein, 9 aa, synthetic construct
  SITE 1: note = D-Tyrosine
  SITE 2: note = D-hydroxyproline
  SITE 6: note = Azaglycine
  SITE 8: note = 4-amino-1-R1-piperidine-4-carboxylic acid
  SITE 9: note = Amidated Tryptophan
YXNTFGLXW

SEQ ID NO: 284 - skipped

SEQ ID NO: 285 - protein, 7 aa, synthetic construct
  SITE 3: note = cyclohexylalanine
  SITE 4: note = Azaglycine
  SITE 6: note = 4-amino-1-R1-piperidine-4-carboxylic acid
  SITE 7: note = Amidated Tryptophan
NTXGLXW

SEQ ID NO: 286 - protein, 8 aa, synthetic construct
  SITE 1: note = D-3-(4-pyridyl)alanine
  SITE 4: note = cyclohexylalanine
  SITE 5: note = Azaglycine
  SITE 7: note = 4-amino-1-R1-piperidine-4-carboxylic acid
  SITE 8: note = Amidated Tryptophan
XNTXGLXW

SEQ ID NO: 287 - protein, 9 aa, synthetic construct
  SITE 1: note = D-Tyrosine
  SITE 2: note = D-3-(4-pyridyl)alanine
  SITE 5: note = cyclohexylalanine
  SITE 6: note = Azaglycine
  SITE 8: note = 4-amino-1-R1-piperidine-4-carboxylic acid
  SITE 9: note = Amidated Tryptophan
YXNTXGLXW

SEQ ID NO: 288 - protein, 9 aa, synthetic construct
  SITE 1: note = D-Tyrosine
  SITE 2: note = D-3-(2-naphthyl)-alanine
  SITE 5: note = cyclohexylalanine
  SITE 6: note = Azaglycine
  SITE 8: note = 4-amino-1-R1-piperidine-4-carboxylic acid
  SITE 9: note = Amidated Tryptophan
YXNTXGLXW

SEQ ID NO: 289 - skipped

SEQ ID NO: 290 - protein, 7 aa, synthetic construct
  SITE 1: note = D-Tyrosine
  SITE 5: note = (S)-2-(4-((S)-1-amino-2-phenylethyl)-1H-1,2,3-triaz ol-1-yl)-4-methylpentanoic acid
  SITE 6: note = 4-amino-1-R1-piperidine-4-carboxylic acid
  SITE 7: note = Amidated Tryptophan
YPNTXXW

SEQ ID NO: 291-301 - skipped (11 entries)

SEQ ID NO: 302 - protein, 8 aa, synthetic construct
  SITE 1: note = D-Tyrosine
  SITE 2: note = D-Tryptophan
  SITE 6: note = 2-(2-aminoethoxy)-[1,1'-biphenyl]-4-carboxylic acid
  SITE 7: note = 4-amino-1-R1-piperidine-4-carboxylic acid
  SITE 8: note = Amidated Tryptophan
YWNTFXXW

SEQ ID NO: 303-305 - skipped (3 entries)

SEQ ID NO: 306 - protein, 9 aa, synthetic construct
  SITE 1: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
KWNTFGLXW

SEQ ID NO: 307 - protein, 8 aa, synthetic construct
  SITE 1: note = (3-(2-(2-methoxyethoxy)ethoxy)propanoyl)-L-Lysine bound to an optional linker and chelating moiety or a radionuclide complex
  SITE 5: note = Azaglycine
  SITE 7: note = 4-amino-1-R1-piperidine-4-carboxylic acid
  SITE 8: note = Amidated Tryptophan
KNTFGLXW

SEQ ID NO: 308 - protein, 9 aa, synthetic construct
  SITE 1: note = D-Tyrosine
  SITE 2: note = Lysine bound to an optional linker and chelating moiety or a radionuclide complex
  SITE 5: note = 3-Fluorophenylalanine
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 309 - protein, 9 aa, synthetic construct
  SITE 1: note = D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 310 - protein, 9 aa, synthetic construct
  SITE 1: note = Ac-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 311 - protein, 9 aa, synthetic construct
  SITE 1: note = octanoic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 312 - protein, 9 aa, synthetic construct
  SITE 1: note = dodecanoic acid acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 313 - protein, 9 aa, synthetic construct
  SITE 1: note = palmitic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 314 - protein, 9 aa, synthetic construct
  SITE 1: note = 3-(4-iodophenyl)propanoic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 315 - protein, 10 aa, synthetic construct
  SITE 1: note = (3-fluoro-4-methylbenzyl)glycine
  SITE 2: note = D-Tyrosine
  SITE 3: note = Lysine bound to an optional linker and chelating moiety or a radionuclide complex
  SITE 7: note = Azaglycine
  SITE 9: note = 4-amino-1-R1-piperidine-4-carboxylic acid
  SITE 10: note = Amidated Tryptophan
GYKNTFGLXW

SEQ ID NO: 316 - protein, 9 aa, synthetic construct
  SITE 1: note = 3-(2-(2-hydroxyethoxy)ethoxy)propanoic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 317 - protein, 9 aa, synthetic construct
  SITE 1: note = 3-(2-(2-methoxyethoxy)ethoxy)propanoic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 318 - protein, 9 aa, synthetic construct
  SITE 1: note = 3-(2-(2-(3-(p-tolyl)propanamido)ethoxy)ethoxy)propa noic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 319 - protein, 9 aa, synthetic construct
  SITE 1: note = 3-(2-(2-(3-(4-iodophenyl)propanamido)ethoxy)ethoxy) propanoic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 320 - protein, 9 aa, synthetic construct
  SITE 1: note = 1-(3-fluoro-4-methylphenyl)-4-oxo-8,11-dioxa-2,5-di azatetradecan-14-oic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 321 - protein, 10 aa, synthetic construct
  SITE 1: note = 3-(p-tolyl)propanoyl)-L-glutamic acid
  SITE 2: note = D-Tyrosine
  SITE 3: note = Lysine bound to an optional linker and chelating moiety or a radionuclide complex
  SITE 7: note = Azaglycine
  SITE 9: note = 4-amino-1-R1-piperidine-4-carboxylic acid
  SITE 10: note = Amidated Tryptophan
XYKNTFGLXW

SEQ ID NO: 322 - protein, 9 aa, synthetic construct
  SITE 1: note = 2,5,8,11,14,17,20,23,26,29,32,35,38,41,44,47,50,53, 56,59,62,65,68,71,74-pentacosaoxaheptaheptacontan-77-oic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 323 - protein, 9 aa, synthetic construct
  SITE 1: note = D-Tyrosine
  SITE 2: note = D-Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 324 - protein, 9 aa, synthetic construct
  SITE 1: note = Octanoic acid-D-Tyrosine
  SITE 2: note = D-Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 325 - protein, 9 aa, synthetic construct
  SITE 1: note = 3-(2-(2-(3-(4-iodophenyl)propanamido)ethoxy)ethoxy) propanoic acid-D-Tyrosine
  SITE 2: note = D-Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 326 - protein, 9 aa, synthetic construct
  SITE 1: note = 3-(2-(2-(3-(p-tolyl)propanamido)ethoxy)ethoxy)propa noic acid-D-Tyrosine
  SITE 2: note = D-Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 327 - protein, 10 aa, synthetic construct
  SITE 1: note = 3-fluoro-4-methylbenzyl)glycine
  SITE 2: note = D-Tyrosine
  SITE 3: note = D-Lysine bound to an optional linker and chelating moiety or a radionuclide complex
  SITE 7: note = Azaglycine
  SITE 9: note = 4-amino-1-R1-piperidine-4-carboxylic acid
  SITE 10: note = Amidated Tryptophan
GYKNTFGLXW

SEQ ID NO: 328 - protein, 9 aa, synthetic construct
  SITE 1: note = 1-(3-fluoro-4-methylphenyl)-4-oxo-8,11-dioxa-2,5-di azatetradecan-14-oic acid-D-Tyrosine
  SITE 2: note = D-Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tryptophan
YKNTFGLXW

SEQ ID NO: 329 - protein, 10 aa, synthetic construct
  SITE 1: note = gamma-D-Glutamic Acid
  SITE 2: note = D-Tyrosine
  SITE 3: note = Lysine bound to an optional linker and chelating moiety or a radionuclide complex
  SITE 7: note = Azaglycine
  SITE 9: note = 4-amino-1-R1-piperidine-4-carboxylic acid
  SITE 10: note = Amidated Tryptophan
EYKNTFGLXW

SEQ ID NO: 330 - protein, 10 aa, synthetic construct
  SITE 1: note = 3-(4-iodophenyl)propanoic acid Gamma-D-Glutamic acid
  SITE 2: note = D-Tyrosine
  SITE 3: note = Lysine bound to an optional linker and chelating moiety or a radionuclide complex
  SITE 7: note = Azaglycine
  SITE 9: note = 4-amino-1-R1-piperidine-4-carboxylic acid
  SITE 10: note = Amidated Tryptophan
EYKNTFGLXW

SEQ ID NO: 331 - protein, 7 aa, synthetic construct
  SITE 1: note = Lysine bound to an optional linker and chelating moiety or a radionuclide complex
  SITE 4: note = Azaglycine
  SITE 6: note = 4-amino-1-R1-piperidine-4-carboxylic acid
  SITE 7: note = Amidated 3-(4-pyridyl)alanine
KTFGLXX

SEQ ID NO: 332 - protein, 7 aa, synthetic construct
  SITE 1: note = 3-(2-(2-hydroxyethoxy)ethoxy)propanoic acid Lysine bound to an optional linker and chelating moiety or a radionuclide complex
  SITE 4: note = Azaglycine
  SITE 6: note = 4-amino-1-R1-piperidine-4-carboxylic acid
  SITE 7: note = Amidated 3-(4-pyridyl)alanine
KTFGLXX

SEQ ID NO: 333 - protein, 7 aa, synthetic construct
  SITE 1: note = D-Lysine bound to an optional linker and chelating moiety or a radionuclide complex
  SITE 4: note = Azaglycine
  SITE 6: note = 4-amino-1-R1-piperidine-4-carboxylic acid
  SITE 7: note = Amidated 3-(4-pyridyl)alanine
KTFGLXX

SEQ ID NO: 334 - protein, 7 aa, synthetic construct
  SITE 1: note = 3-(2-(2-hydroxyethoxy)ethoxy)propanoic acid D-Lysine bound to an optional linker and chelating moiety or a radionuclide complex
  SITE 4: note = Azaglycine
  SITE 6: note = 4-amino-1-R1-piperidine-4-carboxylic acid
  SITE 7: note = Amidated 3-(4-pyridyl)alanine
KTFGLXX

SEQ ID NO: 335 - protein, 8 aa, synthetic construct
  SITE 1: note = Lysine bound to an optional linker and chelating moiety or a radionuclide complex
  SITE 5: note = Azaglycine
  SITE 7: note = 4-amino-1-R1-piperidine-4-carboxylic acid
  SITE 8: note = Amidated 3-(4-pyridyl)alanine
KNTFGLXX

SEQ ID NO: 336 - protein, 8 aa, synthetic construct
  SITE 1: note = Octanoic acid Lysine bound to an optional linker and chelating moiety or a radionuclide complex
  SITE 5: note = Azaglycine
  SITE 7: note = 4-amino-1-R1-piperidine-4-carboxylic acid
  SITE 8: note = Amidated 3-(4-pyridyl)alanine
KNTFGLXX

SEQ ID NO: 337 - protein, 8 aa, synthetic construct
  SITE 1: note = 3-(2-(2-hydroxyethoxy)ethoxy)propanoic acid Lysine bound to an optional linker and chelating moiety or a radionuclide complex
  SITE 5: note = Azaglycine
  SITE 7: note = 4-amino-1-R1-piperidine-4-carboxylic acid
  SITE 8: note = Amidated 3-(4-pyridyl)alanine
KNTFGLXX

SEQ ID NO: 338 - protein, 8 aa, synthetic construct
  SITE 1: note = 3-(2-(2-methoxyethoxy)ethoxy)propanoic acid Lysine bound to an optional linker and chelating moiety or a radionuclide complex
  SITE 5: note = Azaglycine
  SITE 7: note = 4-amino-1-R1-piperidine-4-carboxylic acid
  SITE 8: note = Amidated 3-(4-pyridyl)alanine
KNTFGLXX

SEQ ID NO: 339 - protein, 8 aa, synthetic construct
  SITE 1: note = 3-(2-(2-(3-(p-tolyl)propanamido)ethoxy)ethoxy)propa noic acid Lysine bound to an optional linker and chelating moiety or a radionuclide complex
  SITE 5: note = Azaglycine
  SITE 7: note = 4-amino-1-R1-piperidine-4-carboxylic acid
  SITE 8: note = Amidated 3-(4-pyridyl)alanine
KNTFGLXX

SEQ ID NO: 340 - protein, 8 aa, synthetic construct
  SITE 1: note = 3-(2-(2-(3-(4-iodophenyl)propanamido)ethoxy)ethoxy) propanoic acid Lysine bound to an optional linker and chelating moiety or a radionuclide complex
  SITE 5: note = Azaglycine
  SITE 7: note = 4-amino-1-R1-piperidine-4-carboxylic acid
  SITE 8: note = Amidated 3-(4-pyridyl)alanine
KNTFGLXX

SEQ ID NO: 341 - protein, 8 aa, synthetic construct
  SITE 1: note = 1-hydroxy-3,6,9,12,15,18,21,24-octaoxaheptacosan-27 -oic acid-Lysine bound to an optional linker and chelating moiety or a radionuclide complex
  SITE 5: note = Azaglycine
  SITE 7: note = 4-amino-1-R1-piperidine-4-carboxylic acid
  SITE 8: note = Amidated 3-(4-pyridyl)alanine
KNTFGLXX

SEQ ID NO: 342 - protein, 8 aa, synthetic construct
  SITE 1: note = 2,5,8,11,14,17,20,23,26,29,32,35,38,41,44,47,50,53, 56,59,62,65,68,71,74-pentacosaoxaheptaheptacontan-77-oic acid Lysine bound to an optional linker and chelating moiety or a radionuclide complex
  SITE 5: note = Azaglycine
  SITE 7: note = 4-amino-1-R1-piperidine-4-carboxylic acid
  SITE 8: note = Amidated 3-(4-pyridyl)alanine
KNTFGLXX

SEQ ID NO: 343 - protein, 8 aa, synthetic construct
  SITE 1: note = Lysine bound to an optional linker and chelating moiety or a radionuclide complex
  SITE 5: note = Azaglycine
  SITE 7: note = 4-amino-1-R1-piperidine-4-carboxylic acid
  SITE 8: note = Amidated 3-(4-pyridyl)alanine
KETFGLXX

SEQ ID NO: 344 - protein, 8 aa, synthetic construct
  SITE 1: note = 3-(2-(2-hydroxyethoxy)ethoxy)propanoic acid-Lysine bound to an optional linker and chelating moiety or a radionuclide complex
  SITE 5: note = Azaglycine
  SITE 7: note = 4-amino-1-R1-piperidine-4-carboxylic acid
  SITE 8: note = Amidated 3-(4-pyridyl)alanine
KETFGLXX

SEQ ID NO: 345 - protein, 9 aa, synthetic construct
  SITE 1: note = D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Phenylalanine
YKNTFGLXF

SEQ ID NO: 346 - protein, 9 aa, synthetic construct
  SITE 1: note = 3-(2-(2-(3-(p-tolyl)propanamido)ethoxy)ethoxy)propa noic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Phenylalanine
YKNTFGLXF

SEQ ID NO: 347 - protein, 9 aa, synthetic construct
  SITE 1: note = 3-(2-(2-(3-(4-iodophenyl)propanamido)ethoxy)ethoxy) propanoic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Phenylalanine
YKNTFGLXF

SEQ ID NO: 348 - protein, 9 aa, synthetic construct
  SITE 1: note = D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tyrosine
YKNTFGLXY

SEQ ID NO: 349 - protein, 9 aa, synthetic construct
  SITE 1: note = palmitic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tyrosine
YKNTFGLXY

SEQ ID NO: 350 - protein, 9 aa, synthetic construct
  SITE 1: note = dodecanoic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tyrosine
YKNTFGLXY

SEQ ID NO: 351 - protein, 9 aa, synthetic construct
  SITE 1: note = dodecanoic acid-D-Tyrosine
  SITE 2: note = D-Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tyrosine
YKNTFGLXY

SEQ ID NO: 352 - protein, 9 aa, synthetic construct
  SITE 1: note = dodecanoic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tyrosine
YKNTFGLXY

SEQ ID NO: 353 - protein, 9 aa, synthetic construct
  SITE 1: note = 3-(2-(2-(3-(4-iodophenyl)propanamido)ethoxy)ethoxy) propanoic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated Tyrosine
YKNTFGLXY

SEQ ID NO: 354 - protein, 9 aa, synthetic construct
  SITE 1: note = D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated 4-cyano phenylalanine
YKNTFGLXF

SEQ ID NO: 355 - protein, 9 aa, synthetic construct
  SITE 1: note = 3-(2-(2-(3-(4-iodophenyl)propanamido)ethoxy)ethoxy) propanoic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated 4-cyano phenylalanine
YKNTFGLXF

SEQ ID NO: 356 - protein, 9 aa, synthetic construct
  SITE 1: note = 3-(2-(2-(3-(p-tolyl)propanamido)ethoxy)ethoxy)propa noic acid-D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated 4-cyano phenylalanine
YKNTFGLXF

SEQ ID NO: 357 - protein, 9 aa, synthetic construct
  SITE 1: note = D-Tyrosine
  SITE 2: note = Lysine bound to an optional linker and chelating moiety or a radionuclide complex
  SITE 5: note = cyclohexylalanine
  SITE 6: note = Azaglycine
  SITE 8: note = 4-amino-1-R1-piperidine-4-carboxylic acid
  SITE 9: note = Amidated Tryptophan
YKNTXGLXW

SEQ ID NO: 358 - protein, 9 aa, synthetic construct
  SITE 1: note = 3-(2-(2-(3-(p-tolyl)propanamido)ethoxy)ethoxy)propa noic acid-D-Tyrosine
  SITE 2: note = Lysine bound to an optional linker and chelating moiety or a radionuclide complex
  SITE 5: note = cyclohexylalanine
  SITE 6: note = Azaglycine
  SITE 8: note = 4-amino-1-R1-piperidine-4-carboxylic acid
  SITE 9: note = Amidated Tryptophan
YKNTXGLXW

SEQ ID NO: 359 - protein, 9 aa, synthetic construct
  SITE 1: note = 3-(2-(2-(3-(4-iodophenyl)propanamido)ethoxy)ethoxy) propanoic acid-D-Tyrosine
  SITE 2: note = Lysine bound to an optional linker and chelating moiety or a radionuclide complex
  SITE 5: note = cyclohexylalanine
  SITE 6: note = Azaglycine
  SITE 8: note = 4-amino-1-R1-piperidine-4-carboxylic acid
  SITE 9: note = Amidated Tryptophan
YKNTXGLXW

SEQ ID NO: 360 - protein, 9 aa, synthetic construct
  SITE 1: note = octanoic acid-D-Tyrosine
  SITE 2: note = Lysine bound to an optional linker and chelating moiety or a radionuclide complex
  SITE 5: note = cyclohexylalanine
  SITE 6: note = Azaglycine
  SITE 8: note = 4-amino-1-R1-piperidine-4-carboxylic acid
  SITE 9: note = Amidated Tryptophan
YKNTXGLXW

SEQ ID NO: 361 - protein, 9 aa, synthetic construct
  SITE 1: note = 3-(2-(2-(3-(p-tolyl)propanamido)ethoxy)ethoxy)propa noic acid-D-Tyrosine
  SITE 2: note = D-Lysine bound to an optional linker and chelating moiety or a radionuclide complex
  SITE 5: note = cyclohexylalanine
  SITE 6: note = Azaglycine
  SITE 8: note = 4-amino-1-R1-piperidine-4-carboxylic acid
  SITE 9: note = Amidated Tryptophan
YKNTXGLXW

SEQ ID NO: 362 - skipped

SEQ ID NO: 363 - protein, 7 aa, synthetic construct
  SITE 1: note = D-Asparagine
  SITE 2: note = D-Asparagine
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
NNFGLXW

SEQ ID NO: 364 - protein, 7 aa, synthetic construct
  SITE 1: note = D-Lysine
  SITE 2: note = D-Asparagine
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
KNFGLXW

SEQ ID NO: 365 - protein, 6 aa, synthetic construct
  SITE 1: note = D-Asparagine
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
NFGLXW

SEQ ID NO: 366 - protein, 10 aa, synthetic construct
  SITE 1: note = D-Tyrosine
  SITE 2: note = D-Histidine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
YHWNTFGLXW

SEQ ID NO: 367 - protein, 9 aa, synthetic construct
  SITE 1: note = D-Tyrosine
  SITE 5: note = 3-Fluorophenylalanine
  SITE 6: note = Azaglycine
  SITE 8: note = 4-amino-1-R1-piperidine-4-carboxylic acid
  SITE 9: note = Amidated Tryptophan
YWNTFGLXW

SEQ ID NO: 368 - protein, 7 aa, synthetic construct
  SITE 1: note = D-Glutamic Acid
  SITE 2: note = D-Asparagine
  SITE 4: note = Azaglycine
  SITE 6: note = 4-amino-1-R1-piperidine-4-carboxylic acid
  SITE 7: note = Amidated Tryptophan
ENFGLXW

SEQ ID NO: 369 - protein, 8 aa, synthetic construct
  SITE 1: note = D-Tryptophan
  SITE 5: note = Azaglycine
  SITE 7: note = 4-amino-1-R1-piperidine-4-carboxylic acid
  SITE 8: note = Amidated Tryptophan
WNTFGLXW

SEQ ID NO: 370 - protein, 9 aa, synthetic construct
  SITE 1: note = D-Tyrosine
  SITE 2: note = Azaglycine
  SITE 6: note = Azaglycine
  SITE 8: note = 4-amino-1-R1-piperidine-4-carboxylic acid
  SITE 9: note = Amidated Tryptophan
YGNTFGLXW

SEQ ID NO: 371 - protein, 9 aa, synthetic construct
  SITE 1: note = D-Tyrosine
  SITE 2: note = D-Tryptophan
  SITE 6: note = Azaglycine
  SITE 8: note = 4-amino-1-R1-piperidine-4-carboxylic acid
  SITE 9: note = Amidated Phenylalanine
YWNAFGLXF

SEQ ID NO: 372 - protein, 9 aa, synthetic construct
  SITE 1: note = D-4-iodophenylalanine
  SITE 2: note = D-Tryptophan
  SITE 6: note = Azaglycine
  SITE 8: note = 4-amino-1-R1-piperidine-4-carboxylic acid
  SITE 9: note = Amidated Tryptophan
FWNTFGLXW

SEQ ID NO: 373 - protein, 8 aa, synthetic construct
  SITE 1: note = D-beta-glutamic acid
  SITE 4: note = 3-Fluorophenylalanine
  SITE 5: note = Azaglycine
  SITE 7: note = 4-amino-1-R1-piperidine-4-carboxylic acid
  SITE 8: note = Amidated 3-(4-pyridyl)alanine
ENTFGLXX

SEQ ID NO: 374 - protein, 8 aa, synthetic construct
  SITE 1: note = D-3-(2-naphthyl)-alanine
  SITE 5: note = Azaglycine
  SITE 7: note = 4-amino-1-R1-piperidine-4-carboxylic acid
  SITE 8: note = Amidated Phenylalanine
XNTFGLXF

SEQ ID NO: 375 - protein, 9 aa, synthetic construct
  SITE 1: note = D-Tyrosine
  SITE 2: note = D-3-(2-Naphthyl)alanine
  SITE 6: note = Azaglycine
  SITE 8: note = 4-amino-1-R1-piperidine-4-carboxylic acid
  SITE 9: note = Amidated Tryptophan
YXNTFGLXW

SEQ ID NO: 376 - protein, 9 aa, synthetic construct
  SITE 1: note = D-Tyrosine
  SITE 2: note = 4-benzoylphenylalanine
  SITE 6: note = Azaglycine
  SITE 8: note = 4-amino-1-R1-piperidine-4-carboxylic acid
  SITE 9: note = Amidated D-Tryptophan
YXNTFGLXW

SEQ ID NO: 377 - protein, 9 aa, synthetic construct
  SITE 1: note = D-Tyrosine
  SITE 2: note = D-Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated D-Tryptophan
YKNTFGLXW

SEQ ID NO: 378 - protein, 9 aa, synthetic construct
  SITE 1: note = D-Tyrosine
  SITE 2: note = D-Lysine bound to an optional linker and chelating moiety or a radionuclide complex
  SITE 6: note = cyclohexylalanine
  SITE 8: note = Azaglycine
  SITE 9: note = Amidated D-Tryptophan
YKNTXGLXW

SEQ ID NO: 379 - protein, 9 aa, synthetic construct
  SITE 1: note = D-Tyrosine
  SITE 2: note = Lysine bound to an optional linker and chelating moiety or a radionuclide complex
  SITE 5: note = cyclohexylalanine
  SITE 6: note = Azaglycine
  SITE 8: note = 4-amino-1-R1-piperidine-4-carboxylic acid
  SITE 9: note = Amidated Tryptophan
YKNTXGLXW

SEQ ID NO: 380 - protein, 9 aa, synthetic construct
  SITE 1: note = D-Tyrosine
  SITE 2: note = D-Lysine bound to an optional linker and chelating moiety or a radionuclide complex
  SITE 5: note = cyclohexylalanine
  SITE 6: note = Azaglycine
  SITE 8: note = 4-amino-1-R1-piperidine-4-carboxylic acid
  SITE 9: note = Amidated Tryptophan
YKNTXGLXW

SEQ ID NO: 381 - protein, 9 aa, synthetic construct
  SITE 1: note = D-Tyrosine
  SITE 2: note = Lysine bound to an optional linker and chelating moiety or a radionuclide complex
  SITE 6: note = Azaglycine
  SITE 8: note = 4-amino-1-R1-piperidine-4-carboxylic acid
  SITE 9: note = Amidated D-Tryptophan
YKNTFGLXW

SEQ ID NO: 382 - protein, 9 aa, synthetic construct
  SITE 1: note = D-Tyrosine
  SITE 2: note = D-Tryptophan
  SITE 6: note = Azaglycine
  SITE 8: note = 4-amino-1-R1-piperidine-4-carboxylic acid
  SITE 9: note = Amidated D-Tryptophan
YWNTFGLXW

SEQ ID NO: 383 - protein, 8 aa, synthetic construct
  SITE 5: note = Azaglycine
  SITE 6: note = D-Leucine
  SITE 7: note = 4-amino-1-R1-piperidine-4-carboxylic acid
  SITE 8: note = Amidated Phenylalanine
ENTFGLXF

SEQ ID NO: 384 - protein, 8 aa, synthetic construct
  SITE 1: note = D-Lysine bound to an optional linker and chelating moiety or a radionuclide complex
  SITE 5: note = Azaglycine
  SITE 7: note = 4-amino-1-R1-piperidine-4-carboxylic acid
  SITE 8: note = Amidated 3-(4-pyridyl)alanine
KNTFGLXX

SEQ ID NO: 385 - protein, 8 aa, synthetic construct
  SITE 1: note = 1-hydroxy-3,6,9,12,15,18,21,24-octaoxaheptacosan-27 -oic acid-Lysine bound to an optional linker and chelating moiety or a radionuclide complex
  SITE 5: note = Azaglycine
  SITE 7: note = 4-amino-1-R1-piperidine-4-carboxylic acid
  SITE 8: note = Amidated 3-(4-pyridyl)alanine
KNTFGLXX

SEQ ID NO: 386 - protein, 6 aa, synthetic construct
  SITE 1: note = DOTA-bAla
  SITE 2: note = Sarcosine
  SITE 3: note = Sarcosine
  SITE 4: note = Sarcosine
  SITE 5: note = Sarcosine
  SITE 6: note = Sarcosine
AGGGGG

SEQ ID NO: 387 - protein, 5 aa, synthetic construct
  SITE 1: note = DOTA-D-Ala
  SITE 2: note = D-Alanine
  SITE 3: note = D-Alanine
  SITE 4: note = D-Alanine
  SITE 5: note = D-Alanine
AAAAA

SEQ ID NO: 388 - protein, 6 aa, synthetic construct
  SITE 1: note = DOTA-Beta-Alanine
  SITE 2: note = D-Asparagine
  SITE 3: note = D-Asparagine
  SITE 4: note = D-Asparagine
  SITE 5: note = D-Asparagine
  SITE 6: note = D-Asparagine
ANNNNN

SEQ ID NO: 389 - protein, 6 aa, synthetic construct
  SITE 1: note = 2,2',2'',2'''-(1,4,7,10-tetraazacyclododecane-1,4,7 ,10-tetrayl)tetraacetic acid-Beta-Alanine
  SITE 2: note = D-Asparagine
  SITE 3: note = D-Asparagine
  SITE 4: note = D-Glutamic Acid
  SITE 5: note = D-Glutamic Acid
  SITE 6: note = D-Asparagine
ANNEEN

SEQ ID NO: 390 - protein, 6 aa, synthetic construct
  SITE 1: note = 2,2',2'',2'''-(1,4,7,10-tetraazacyclododecane-1,4,7 ,10-tetrayl)tetraacetic acid-Beta-Alanine
  SITE 2: note = D-Asparagine
  SITE 3: note = D-Asparagine
  SITE 4: note = D-Lysine
  SITE 5: note = D-Glutamic Acid
  SITE 6: note = D-Asparagine
ANNKEN

SEQ ID NO: 391 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYWNTFGLXW

SEQ ID NO: 392 - protein, 10 aa, synthetic construct
  SITE 1: note = indium(III) 2,2',2''-(10-(2-((5-carboxypentyl)amino )-2-oxoethyl)-1,4,7,10-tetraazacyclododecane-1,4,7-triyl)t riacetate
  SITE 2: note = D-Tyrosine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYWNTFGLXW

SEQ ID NO: 393 - protein, 10 aa, synthetic construct
  SITE 1: note = Lutetium (III) 2,2',2''-(10-(2-((5-carboxypentyl)am ino)-2-oxoethyl)-1,4,7,10-tetraazacyclododecane-1,4,7-triy l)triacetate
  SITE 2: note = D-Tyrosine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYWNTFGLXW

SEQ ID NO: 394 - protein, 10 aa, synthetic construct
  SITE 1: note = gallium(III) 2,2',2''-(10-(2-((5-carboxypentyl)amin o)-2-oxoethyl)-1,4,7,10-tetraazacyclododecane-1,4,7-triyl) triacetate
  SITE 2: note = D-Tyrosine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYWNTFGLXW

SEQ ID NO: 395 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 396 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 5: note = 4-amino-1-carbamimidoylpiperidine-4-carboxylic acid
  SITE 6: note = Amidated Tryptophan
XFGLXW

SEQ ID NO: 397 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = alpha-Me-Phenylalanine
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 398 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 3: note = Azaglycine
  SITE 5: note = 4-amino-1-carbamimidoylpiperidine-4-carboxylic acid
  SITE 6: note = Amidated Tryptophan
XFGLXW

SEQ ID NO: 399 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-carbamimidoylpiperidine-4-carboxylic acid
  SITE 10: note = Amidated Tryptophan
XYWNTFGLXW

SEQ ID NO: 400 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 3: note = Azaglycine
  SITE 5: note = 4-amino-1-(4-(p-tolyl)butanoyl)piperidine-4-carboxy lic acid
  SITE 6: note = Amidated Tryptophan
XFGLXW

SEQ ID NO: 401-402 - skipped (2 entries)

SEQ ID NO: 403 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 404 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 5: note = 4-amino-1-((3-fluoro-4-methylbenzyl)glycyl)piperidi ne-4-carboxamide
  SITE 6: note = Amidated Tyrosine
XFGLXY

SEQ ID NO: 405 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 3: note = Sarcosine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 406 - protein, 11 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Alanine
  SITE 3: note = D-Alanine
  SITE 4: note = D-Alanine
  SITE 5: note = D-Alanine
  SITE 6: note = D-Alanine
  SITE 8: note = Azaglycine
  SITE 10: note = 4-amino-1-((3-fluoro-4-methylbenzyl)glycyl)piperidi ne-4-carboxamide
  SITE 11: note = Amidated Tryptophan
XAAAAAFGLXW

SEQ ID NO: 407 - protein, 11 aa, synthetic construct
  SITE 1: note = DOTA-beta-Alanine
  SITE 2: note = D-Asparagine
  SITE 3: note = D-Asparagine
  SITE 4: note = D-Asparagine
  SITE 5: note = D-Asparagine
  SITE 6: note = D-Asparagine
  SITE 8: note = Azaglycine
  SITE 10: note = 4-amino-1-((3-fluoro-4-methylbenzyl)glycyl)piperidi ne-4-carboxamide
  SITE 11: note = Amidated Tryptophan
ANNNNNFGLXW

SEQ ID NO: 408 - protein, 11 aa, synthetic construct
  SITE 1: note = 2,2',2'',2'''-(1,4,7,10-tetraazacyclododecane-1,4,7 ,10-tetrayl)tetraacetic acid-beta-Alanine
  SITE 2: note = D-Asparagine
  SITE 3: note = D-Asparagine
  SITE 4: note = D-Glutamic Acid
  SITE 5: note = D-Glutamic Acid
  SITE 6: note = D-Asparagine
  SITE 8: note = Azaglycine
  SITE 10: note = 4-amino-1-((3-fluoro-4-methylbenzyl)glycyl)piperidi ne-4-carboxamide
  SITE 11: note = Amidated Tryptophan
ANNEENFGLXW

SEQ ID NO: 409 - protein, 11 aa, synthetic construct
  SITE 1: note = 2,2',2'',2'''-(1,4,7,10-tetraazacyclododecane-1,4,7 ,10-tetrayl)tetraacetic acid-beta-Alanine
  SITE 2: note = D-Asparagine
  SITE 3: note = D-Asparagine
  SITE 4: note = D-Lysine
  SITE 5: note = D-Glutamic Acid
  SITE 6: note = D-Asparagine
  SITE 8: note = Azaglycine
  SITE 10: note = 4-amino-1-((3-fluoro-4-methylbenzyl)glycyl)piperidi ne-4-carboxamide
  SITE 11: note = Amidated Tryptophan
ANNKENFGLXW

SEQ ID NO: 410 - protein, 7 aa, synthetic construct
  SITE 1: note = 2,2',2'',2'''-(1,4,7,10-tetraazacyclododecane-1,4,7 ,10-tetrayl)tetraacetic acid-Sarcosine
  SITE 2: note = Sarcosine
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
GGFGLXW

SEQ ID NO: 411 - protein, 7 aa, synthetic construct
  SITE 1: note = 2,2',2'',2'''-(1,4,7,10-tetraazacyclododecane-1,4,7 ,10-tetrayl)tetraacetic acid-D-Asparagine
  SITE 2: note = D-Asparagine
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
NNFGLXW

SEQ ID NO: 412 - protein, 7 aa, synthetic construct
  SITE 1: note = 2,2',2'',2'''-(1,4,7,10-tetraazacyclododecane-1,4,7 ,10-tetrayl)tetraacetic acid-D-Lys
  SITE 2: note = D-Asparagine
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
KNFGLXW

SEQ ID NO: 413 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYPNTFGLXW

SEQ ID NO: 414 - protein, 6 aa, synthetic construct
  SITE 1: note = 2,2',2'',2'''-(1,4,7,10-tetraazacyclododecane-1,4,7 ,10-tetrayl)tetraacetic acid-D-Asn
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
NFGLXW

SEQ ID NO: 415 - protein, 7 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 3: note = NHCH2-Phenylalanine-C(=O)-
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFFGLXW

SEQ ID NO: 416 - protein, 9 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = D-3-(4-pyridyl)alanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YXNTFGLXW

SEQ ID NO: 417 - protein, 10 aa, synthetic construct
  SITE 1: note = 2,2',2'',2'''-(1,4,7,10-tetraazacyclododecane-1,4,7 ,10-tetrayl)tetraacetic acid-2-(4-aminopiperidin-1-yl)acetic acid
  SITE 2: note = D-Tyrosine
  SITE 6: note = 3-fluorophenylalanine
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYENTFGLXW

SEQ ID NO: 418 - protein, 6 aa, synthetic construct
  SITE 1: note = 2,2',2'',2'''-(1,4,7,10-tetraazacyclododecane-1,4,7 ,10-tetrayl)tetraacetic acid-NHCH2-Chx-C(=O)-
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 419 - protein, 8 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
XNTFGLXW

SEQ ID NO: 420 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 6: note = 3-fluorophenylalanine
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYKNTFGLXW

SEQ ID NO: 421 - protein, 6 aa, synthetic construct
  SITE 1: note = 2,2',2'',2'''-(1,4,7,10-tetraazacyclododecane-1,4,7 ,10-tetrayl)tetraacetic acid-APM
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 422 - protein, 10 aa, synthetic construct
  SITE 1: note = 2,2',2'',2'''-(1,4,7,10-tetraazacyclododecane-1,4,7 ,10-tetrayl)tetraacetic acid-2-(4-aminopiperidin-1-yl)acetic acid
  SITE 2: note = D-Tyrosine
  SITE 3: note = D-3-(4-pyridyl)alanine
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYXNTFGLXW

SEQ ID NO: 423 - protein, 9 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tryptophan
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
XWNTFGLXW

SEQ ID NO: 424 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(4-pyridyl)alanine
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYXNTFGLXW

SEQ ID NO: 425 - protein, 11 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Histidine
  SITE 4: note = D-Tryptophan
  SITE 8: note = Azaglycine
  SITE 10: note = 4-amino-1-((3-fluoro-4-methylbenzyl)glycyl)piperidi ne-4-carboxamide
  SITE 11: note = Amidated Tryptophan
XYHWNTFGLXW

SEQ ID NO: 426 - protein, 11 aa, synthetic construct
  SITE 1: note = 2,2',2'',2'''-(1,4,7,10-tetraazacyclododecane-1,4,7 ,10-tetrayl)tetraacetic acid-APM
  SITE 2: note = D-Tyrosine
  SITE 3: note = D-Histidine
  SITE 4: note = D-Tryptophan
  SITE 8: note = Azaglycine
  SITE 10: note = 4-amino-1-((3-fluoro-4-methylbenzyl)glycyl)piperidi ne-4-carboxamide
  SITE 11: note = Amidated Tryptophan
XYHWNTFGLXW

SEQ ID NO: 427 - protein, 10 aa, synthetic construct
  SITE 1: note = 2,2',2'',2'''-(1,4,7,10-tetraazacyclododecane-1,4,7 ,10-tetrayl)tetraacetic acid-APM
  SITE 2: note = D-Tyrosine
  SITE 3: note = D-Tryptophan
  SITE 6: note = 3-fluorophenylalanine
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYWNTFGLXW

SEQ ID NO: 428 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 6: note = 3-fluorophenylalanine
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYWNTFGLXW

SEQ ID NO: 429 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 6: note = 3-fluorophenylalanine
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYENTFGLXW

SEQ ID NO: 430 - protein, 6 aa, synthetic construct
  SITE 1: note = 2,2',2'',2'''-(1,4,7,10-tetraazacyclododecane-1,4,7 ,10-tetrayl)tetraacetic acid-Lys
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
KFGLXW

SEQ ID NO: 431 - protein, 7 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = NHCH2-Chx-C(=O)
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
XXFGLXW

SEQ ID NO: 432 - skipped

SEQ ID NO: 433 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 3: note = Azaglycine
  SITE 4: note = D-Leucine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 434 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = Sarcosine
  SITE 3: note = Sarcosine
  SITE 4: note = Sarcosine
  SITE 5: note = Sarcosine
  SITE 9: note = 4-amino-1-carbamimidoylpiperidine-4-carboxylic acid
  SITE 10: note = Amidated Tryptophan
XGGGGFGLXW

SEQ ID NO: 435 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 3: note = Sarcosine
  SITE 4: note = Methyl-leucine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 436 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 3: note = Azaglycine
  SITE 5: note = 4-amino-1-((3-fluoro-4-methylbenzyl)glycyl)piperidi ne-4-carboxamide
  SITE 6: note = Me-Trp-NH2
XFGLXW

SEQ ID NO: 437-438 - skipped (2 entries)

SEQ ID NO: 439 - protein, 9 aa, synthetic construct
  SITE 1: note = 2,2',2'',2'''-(1,4,7,10-tetraazacyclododecane-1,4,7 ,10-tetrayl)tetraacetic acid-Beta-Alanine
  SITE 2: note = Sarcosine
  SITE 3: note = Sarcosine
  SITE 4: note = Sarcosine
  SITE 5: note = Sarcosine
  SITE 6: note = Sarcosine
  SITE 8: note = 4-amino-1-((3-fluoro-4-methylbenzyl)glycyl)piperidi ne-4-carboxamide
  SITE 9: note = Amidated Tryptophan
AGGGGGFXW

SEQ ID NO: 440 - protein, 9 aa, synthetic construct
  SITE 1: note = 2,2',2'',2'''-(1,4,7,10-tetraazacyclododecane-1,4,7 ,10-tetrayl)tetraacetic acid-Beta-Alanine
  SITE 2: note = Sarcosine
  SITE 3: note = Sarcosine
  SITE 4: note = Sarcosine
  SITE 5: note = Sarcosine
  SITE 6: note = Sarcosine
  SITE 7: note = phenylglycine
  SITE 8: note = 4-amino-1-((3-fluoro-4-methylbenzyl)glycyl)piperidi ne-4-carboxamide
  SITE 9: note = Amidated Tryptophan
AGGGGGGXW

SEQ ID NO: 441 - protein, 5 aa, synthetic construct
  SITE 1: note = 2,2',2'',2'''-(1,4,7,10-tetraazacyclododecane-1,4,7 ,10-tetrayl)tetraacetic acid-Phe
  SITE 2: note = Azaglycine
  SITE 4: note = 4-amino-1-((3-fluoro-4-methylbenzyl)glycyl)piperidi ne-4-carboxamide
  SITE 5: note = Amidated Tryptophan
FGLXW

SEQ ID NO: 442 - protein, 6 aa, synthetic construct
  SITE 1: note = 2,2',2'',2'''-(1,4,7,10-tetraazacyclododecane-1,4,7 ,10-tetrayl)tetraacetic acid-ABA
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 443 - skipped

SEQ ID NO: 444 - protein, 6 aa, synthetic construct
  SITE 1: note = 2,2',2'',2'''-(1,4,7,10-tetraazacyclododecane-1,4,7 ,10-tetrayl)tetraacetic acid-ABA
  SITE 3: note = Sarcosine
  SITE 4: note = Methyl-leucine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 445 - protein, 6 aa, synthetic construct
  SITE 1: note = 2,2',2'',2'''-(1,4,7,10-tetraazacyclododecane-1,4,7 ,10-tetrayl)tetraacetic acid-ABA
  SITE 4: note = N-Me-Leu
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 446 - skipped

SEQ ID NO: 447 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 3: note = Azaglycine
  SITE 5: note = 4-amino-1-((3-fluoro-4-methylbenzyl)glycyl)piperidi ne-4-carboxamide
  SITE 6: note = Amidated Phenylalanine
XFGLXF

SEQ ID NO: 448 - protein, 9 aa, synthetic construct
  SITE 1: note = 2,2',2'',2'''-(1,4,7,10-tetraazacyclododecane-1,4,7 ,10-tetrayl)tetraacetic acid-NH-glycol D-Tyrosine
  SITE 2: note = D-Tryptophan
  SITE 6: note = Azaglycine
  SITE 8: note = 4-amino-1-((3-fluoro-4-methylbenzyl)glycyl)piperidi ne-4-carboxamide
  SITE 9: note = Amidated Phenylalanine
YWNTFGLXF

SEQ ID NO: 449 - protein, 9 aa, synthetic construct
  SITE 1: note = 2,2',2'',2'''-(1,4,7,10-tetraazacyclododecane-1,4,7 ,10-tetrayl)tetraacetic acid-NH-glycol-D-Tyrosine
  SITE 2: note = D-Tryptophan
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YWNTFGLXW

SEQ ID NO: 450 - protein, 9 aa, synthetic construct
  SITE 1: note = 2,2',2'',2'''-(1,4,7,10-tetraazacyclododecane-1,4,7 ,10-tetrayl)tetraacetic acid-NH-glycol D-Tyrosine
  SITE 2: note = 3-(2-Naphthyl)alanine or
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YXNTFGLXW

SEQ ID NO: 451 - protein, 9 aa, synthetic construct
  SITE 1: note = 2,2',2'',2'''-(1,4,7,10-tetraazacyclododecane-1,4,7 ,10-tetrayl)tetraacetic acid-NH-glycol D-Tyrosine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YRNTFGLXW

SEQ ID NO: 452 - protein, 10 aa, synthetic construct
  SITE 1: note = 2,2',2'',2'''-(1,4,7,10-tetraazacyclododecane-1,4,7 ,10-tetrayl)tetraacetic acid-NH-glycol D-Tyrosine
  SITE 2: note = D-Asparagine
  SITE 3: note = D-Arginine
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
YNRNTFGLXW

SEQ ID NO: 453 - protein, 11 aa, synthetic construct
  SITE 1: note = 2,2',2'',2'''-(1,4,7,10-tetraazacyclododecane-1,4,7 ,10-tetrayl)tetraacetic acid-beta-Alanine
  SITE 2: note = Sarcosine
  SITE 3: note = Sarcosine
  SITE 4: note = Sarcosine
  SITE 5: note = Sarcosine
  SITE 6: note = Sarcosine
  SITE 8: note = Azaglycine
  SITE 10: note = 4-amino-1-((3-fluoro-4-methylbenzyl)glycyl)piperidi ne-4-carboxamide
  SITE 11: note = Amidated Tryptophan
AGGGGGFGLXW

SEQ ID NO: 454 - protein, 7 aa, synthetic construct
  SITE 1: note = 2,2',2'',2'''-(1,4,7,10-tetraazacyclododecane-1,4,7 ,10-tetrayl)tetraacetic acid-D-Glu
  SITE 2: note = D-Asparagine
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
ENFGLXW

SEQ ID NO: 455 - protein, 9 aa, synthetic construct
  SITE 1: note = 2,2',2''-(10-(2-((2-(carboxymethoxy)ethyl)amino)-2- oxoethyl)-1,4,7,10-tetraazacyclododecane-1,4,7-triyl)triac etic acid coupled to N-term of D-Tyrosine
  SITE 2: note = D-Alanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YANTFGLXW

SEQ ID NO: 456 - protein, 9 aa, synthetic construct
  SITE 1: note = HO-PEG3-C(=O)-D-Tyrosine
  SITE 2: note = Lysine(1,4,7,10-tetraazacyclododecane-1,4,7,10-tetr aacetic acid)
  SITE 5: note = 3-fluorophenylalanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YKNTFGLXW

SEQ ID NO: 457 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-(4-(3-fluoro-4-methylphenyl)butanoyl)pipe ridine-4-carboxylic acid
  SITE 10: note = Amidated Tryptophan
XYWNTFGLXW

SEQ ID NO: 458 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-(4-(p-tolyl)butanoyl)piperidine-4-carboxy lic acid
  SITE 10: note = Amidated Tryptophan
XYWNTFGLXW

SEQ ID NO: 459 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-(2-(p-tolyl)acetyl)piperidine-4-carboxyli c acid
  SITE 10: note = Amidated Tryptophan
XYWNTFGLXW

SEQ ID NO: 460 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-(2-(4-iodophenyl)acetyl)piperidine-4-carb oxylic acid
  SITE 10: note = Amidated Tryptophan
XYWNTFGLXW

SEQ ID NO: 461 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((4-aminobenzyl)glycyl)piperidine-4-carbo xylic acid
  SITE 10: note = Amidated Tryptophan
XYWNTFGLXW

SEQ ID NO: 462 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-(2-amino-2-(3-fluoro-4-methylphenyl)acety l)piperidine-4-carboxylic acid
  SITE 10: note = Amidated Tryptophan
XYWNTFGLXW

SEQ ID NO: 463 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-(N-(2-aminoethyl)-N-methylglycyl)piperidi ne-4-carboxylic acid
  SITE 10: note = Amidated Tryptophan
XYWNTFGLXW

SEQ ID NO: 464 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((4-cyclopropylbenzyl)glycyl)piperidine-4 -carboxylic acid
  SITE 10: note = Amidated Tryptophan
XYWNTFGLXW

SEQ ID NO: 465 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((4-ethylbenzyl)glycyl)piperidine-4-carbo xylic acid
  SITE 10: note = Amidated Tryptophan
XYWNTFGLXW

SEQ ID NO: 466 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((4-hydroxybenzyl)glycyl)piperidine-4-car boxylic acid
  SITE 10: note = Amidated Tryptophan
XYWNTFGLXW

SEQ ID NO: 467 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((3,5-difluoro-4-methylbenzyl)glycyl)pipe ridine-4-carboxylic acid
  SITE 10: note = Amidated Tryptophan
XYWNTFGLXW

SEQ ID NO: 468 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = (R)-4-amino-1-((R)-(1-(3-fluoro-4-methylphenyl)ethy l)glycyl)piperidine-4-carboxylic acid
  SITE 10: note = Amidated Tryptophan
XYWNTFGLXW

SEQ ID NO: 469 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((3,4-dimethylbenzyl)glycyl)piperidine-4- carboxylic acid
  SITE 10: note = Amidated Tryptophan
XYWNTFGLXW

SEQ ID NO: 470 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((4-chloro-3-fluorobenzyl)glycyl)piperidi ne-4-carboxylic acid
  SITE 10: note = Amidated Tryptophan
XYWNTFGLXW

SEQ ID NO: 471 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((4-carboxybenzyl)glycyl)piperidine-4-car boxylic acid
  SITE 10: note = Amidated Tryptophan
XYWNTFGLXW

SEQ ID NO: 472 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((2,3-difluoro-4-methylbenzyl)glycyl)pipe ridine-4-carboxylic acid
  SITE 10: note = Amidated Tryptophan
XYWNTFGLXW

SEQ ID NO: 473 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((3-fluoro-4-methoxybenzyl)glycyl)piperid ine-4-carboxylic acid
  SITE 10: note = Amidated Tryptophan
XYWNTFGLXW

SEQ ID NO: 474 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((3-chloro-4-methylbenzyl)glycyl)piperidi ne-4-carboxylic acid
  SITE 10: note = Amidated Tryptophan
XYWNTFGLXW

SEQ ID NO: 475 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((3,4,5-trifluorobenzyl)glycyl)piperidine -4-carboxylic acid
  SITE 10: note = Amidated Tryptophan
XYWNTFGLXW

SEQ ID NO: 476 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((4-(methylamino)benzyl)glycyl)piperidine -4-carboxylic acid
  SITE 10: note = Amidated Tryptophan
XYWNTFGLXW

SEQ ID NO: 477 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = (Z)-4-amino-1-(N'-((3-fluoro-4-methylbenzyl)carbamo yl)carbamimidoyl)piperidine-4-carboxylic acid
  SITE 10: note = Amidated Tryptophan
XYWNTFGLXW

SEQ ID NO: 478 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((3-fluoro-4-methylbenzyl)glycyl)piperidi ne-4-carboxylic acid
  SITE 10: note = Amidated Tryptophan
XYWNTFGLXW

SEQ ID NO: 479 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-carbamimidoylpiperidine-4-carboxylic acid(Me)
  SITE 10: note = Amidated Tryptophan
XYWNTFGLXW

SEQ ID NO: 480 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 5: note = 4-amino-1-carbamimidoylpiperidine-4-carboxylic acid(Me)
  SITE 6: note = Amidated Tryptophan
XFGLXW

SEQ ID NO: 481 - protein, 9 aa, synthetic construct
  SITE 1: note = 2,2',2'',2'''-(1,4,7,10-tetraazacyclododecane-1,4,7 ,10-tetrayl)tetraacetic acid-NH-glycol-D-Tyrosine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YPNTFGLXW

SEQ ID NO: 482 - protein, 8 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 6: note = (S)-2-(4-((S)-1-amino-2-phenylethyl)-1H-1,2,3-triaz ol-1-yl)-4-methylpentanoic acid
  SITE 7: note = 4-amino-1-((3-fluoro-4-methylbenzyl)glycyl)piperidi ne-4-carboxamide
  SITE 8: note = Amidated Tryptophan
XYPNTXXW

SEQ ID NO: 483 - protein, 7 aa, synthetic construct
  SITE 1: note = 2,2',2'',2'''-(1,4,7,10-tetraazacyclododecane-1,4,7 ,10-tetrayl)tetraacetic acid-D-Asn
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
NGFGLXW

SEQ ID NO: 484 - protein, 7 aa, synthetic construct
  SITE 1: note = 2,2',2'',2'''-(1,4,7,10-tetraazacyclododecane-1,4,7 ,10-tetrayl)tetraacetic acid-Asn
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
NGFGLXW

SEQ ID NO: 485 - protein, 7 aa, synthetic construct
  SITE 1: note = 2,2',2'',2'''-(1,4,7,10-tetraazacyclododecane-1,4,7 ,10-tetrayl)tetraacetic acid-D-Asn
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
NTFGLXW

SEQ ID NO: 486 - protein, 8 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
XNTFGLXW

SEQ ID NO: 487 - protein, 7 aa, synthetic construct
  SITE 1: note = 2,2',2'',2'''-(1,4,7,10-tetraazacyclododecane-1,4,7 ,10-tetrayl)tetraacetic acid-Asn
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
NTFGLXW

SEQ ID NO: 488 - protein, 8 aa, synthetic construct
  SITE 1: note = 2,2',2'',2'''-(1,4,7,10-tetraazacyclododecane-1,4,7 ,10-tetrayl)tetraacetic acid-D-Trp
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
WNTFGLXW

SEQ ID NO: 489 - protein, 7 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
XWFGLXW

SEQ ID NO: 490 - protein, 7 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = 2-Pal
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
XXFGLXW

SEQ ID NO: 491 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated 4-cyano phenylalanine
XYWNTFGLXF

SEQ ID NO: 492 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated 3-(4-pyridyl)alanine
XYWNTFGLXX

SEQ ID NO: 493 - protein, 10 aa, synthetic construct
  SITE 1: note = indium(III) 2,2',2''-(10-(2-((5-carboxypentyl)amino )-2-oxoethyl)-1,4,7,10-tetraazacyclododecane-1,4,7-triyl)t riacetate
  SITE 2: note = D-Tyrosine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated 3-(4-pyridyl)alanine
XYWNTFGLXX

SEQ ID NO: 494 - protein, 11 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 4: note = D-Tryptophan
  SITE 8: note = Azaglycine
  SITE 10: note = 4-amino-1-((3-fluoro-4-methylbenzyl)glycyl)piperidi ne-4-carboxamide
  SITE 11: note = Amidated 3-(4-pyridyl)alanine
XYNWNTFGLXX

SEQ ID NO: 495 - protein, 11 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Histidine
  SITE 4: note = D-Tryptophan
  SITE 8: note = Azaglycine
  SITE 10: note = 4-amino-1-((3-fluoro-4-methylbenzyl)glycyl)piperidi ne-4-carboxamide
  SITE 11: note = Amidated 3-(4-pyridyl)alanine
XYHWNTFGLXX

SEQ ID NO: 496 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = Azaglycine
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYGNTFGLXW

SEQ ID NO: 497 - protein, 6 aa, synthetic construct
  SITE 1: note = 2,2',2'',2'''-(1,4,7,10-tetraazacyclododecane-1,4,7 ,10-tetrayl)tetraacetic acid-AMBA
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 498 - protein, 7 aa, synthetic construct
  SITE 1: note = 2,2',2'',2'''-(1,4,7,10-tetraazacyclododecane-1,4,7 ,10-tetrayl)tetraacetic acid-AMBA
  SITE 2: note = (1r,4r)-4-(aminomethyl)cyclohexane-1-carboxylic acid
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
XXFGLXW

SEQ ID NO: 499 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated Phenylalanine
XYWNAFGLXF

SEQ ID NO: 500 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated Leucine
XYWNAFGLXL

SEQ ID NO: 501 - protein, 13 aa, synthetic construct
  SITE 1: note = 2,2',2'',2'''-(1,4,7,10-tetraazacyclododecane-1,4,7 ,10-tetrayl)tetraacetic acid-D-CsA
  SITE 2: note = D-CsA
  SITE 3: note = D-CsA
  SITE 4: note = Ahx
  SITE 5: note = D-Tyrosine
  SITE 6: note = D-Tryptophan
  SITE 10: note = Azaglycine
  SITE 12: note = 4-amino-1-((3-fluoro-4-methylbenzyl)glycyl)piperidi ne-4-carboxamide
  SITE 13: note = Amidated Tryptophan
XXXXYWNTFGLXW

SEQ ID NO: 502 - protein, 11 aa, synthetic construct
  SITE 1: note = 2,2',2'',2'''-(1,4,7,10-tetraazacyclododecane-1,4,7 ,10-tetrayl)tetraacetic acid-D-CsA
  SITE 2: note = Ahx
  SITE 3: note = D-Tyrosine
  SITE 4: note = D-Tryptophan
  SITE 8: note = Azaglycine
  SITE 10: note = 4-amino-1-((3-fluoro-4-methylbenzyl)glycyl)piperidi ne-4-carboxamide
  SITE 11: note = Amidated Tryptophan
XXYWNTFGLXW

SEQ ID NO: 503 - protein, 7 aa, synthetic construct
  SITE 1: note = 2,2',2'',2'''-(1,4,7,10-tetraazacyclododecane-1,4,7 ,10-tetrayl)tetraacetic acid-D-CsA
  SITE 2: note = Ahx
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
XXFGLXW

SEQ ID NO: 504 - protein, 9 aa, synthetic construct
  SITE 1: note = 2,2',2'',2'''-(1,4,7,10-tetraazacyclododecane-1,4,7 ,10-tetrayl)tetraacetic acid-AMBA-Met-Val-Lyse-ada-D-Tyrosine
  SITE 2: note = D-Tryptophan
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YWNTFGLXW

SEQ ID NO: 505 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = 3-(2-Naphthyl)alanine or
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYXNTFGLXW

SEQ ID NO: 506 - protein, 11 aa, synthetic construct
  SITE 1: note = (C16-gGlu)
  SITE 2: note = (DOTA-PEG4)-eLys
  SITE 3: note = D-Tyrosine
  SITE 4: note = D-Tryptophan
  SITE 8: note = Azaglycine
  SITE 10: note = 4-amino-1-((3-fluoro-4-methylbenzyl)glycyl)piperidi ne-4-carboxamide
  SITE 11: note = Amidated Tryptophan
XKYWNTFGLXW

SEQ ID NO: 507 - protein, 12 aa, synthetic construct
  SITE 1: note = 2,2',2'',2'''-(1,4,7,10-tetraazacyclododecane-1,4,7 ,10-tetrayl)tetraacetic acid-D-CsA
  SITE 2: note = D-CsA
  SITE 3: note = Ahx
  SITE 4: note = D-Tyrosine
  SITE 5: note = D-Tryptophan
  SITE 9: note = Azaglycine
  SITE 11: note = 4-amino-1-((3-fluoro-4-methylbenzyl)glycyl)piperidi ne-4-carboxamide
  SITE 12: note = Amidated Tryptophan
XXXYWNTFGLXW

SEQ ID NO: 508 - protein, 9 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = 4-iodophenylalanine
  SITE 5: note = 4-iodophenylalanine
  SITE 6: note = Azaglycine
  SITE 8: note = 4-amino-1-carbamimidoylpiperidine-4-carboxylic acid
  SITE 9: note = Amidated 3-(4-pyridyl)alanine
YFNTFGLXX

SEQ ID NO: 509 - protein, 8 aa, synthetic construct
  SITE 1: note = CH3-(Peg)25-C(=O)-Lys(DOTA)
  SITE 5: note = Azaglycine
  SITE 7: note = 4-amino-1-carbamimidoylpiperidine-4-carboxylic acid
  SITE 8: note = Amidated 3-(4-pyridyl)alanine
KNTFGLXX

SEQ ID NO: 510 - protein, 9 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = 3-(9-anthryl)-alanine
  SITE 6: note = Azaglycine
  SITE 7: note = Norleucine
  SITE 8: note = 4-amino-1-carbamimidoylpiperidine-4-carboxylic acid
  SITE 9: note = Amidated 3-(4-pyridyl)alanine
YXNTFGXXX

SEQ ID NO: 511 - protein, 8 aa, synthetic construct
  SITE 1: note = 2,2',2'',2'''-(1,4,7,10-tetraazacyclododecane-1,4,7 ,10-tetrayl)tetraacetic acid-D-CsA
  SITE 2: note = D-CsA
  SITE 3: note = 6-aminohexanoic acid
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
XXXFGLXW

SEQ ID NO: 512 - protein, 9 aa, synthetic construct
  SITE 1: note = 2,2',2'',2'''-(1,4,7,10-tetraazacyclododecane-1,4,7 ,10-tetrayl)tetraacetic acid-D-CsA
  SITE 2: note = D-CsA
  SITE 3: note = D-CsA
  SITE 4: note = 6-aminohexanoic acid
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
XXXXFGLXW

SEQ ID NO: 513 - protein, 6 aa, synthetic construct
  SITE 1: note = 2,2',2'',2'''-(1,4,7,10-tetraazacyclododecane-1,4,7 ,10-tetrayl)tetraacetic acid-AMBA-Met-Val-Lyse-Ada
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 514 - protein, 10 aa, synthetic construct
  SITE 1: note = 2,2',2'',2'''-(1,4,7,10-tetraazacyclododecane-1,4,7 ,10-tetrayl)tetraacetic acid-Met-Val-Lyse-Ada
  SITE 2: note = D-Tyrosine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
XYWNTFGLXW

SEQ ID NO: 515-520 - skipped (6 entries)

SEQ ID NO: 521 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((4-methoxybenzyl)glycyl)piperidine-4-car boxylic acid
  SITE 10: note = Amidated Phenylalanine
XYWNTFGLXF

SEQ ID NO: 522-523 - skipped (2 entries)

SEQ ID NO: 524 - protein, 6 aa, synthetic construct
  SITE 1: note = 2,2',2'',2'''-(1,4,7,10-tetraazacyclododecane-1,4,7 ,10-tetrayl)tetraacetic acid-bAla-Met-Val-Lys-C(=O)-6-aminohexanoic acid
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 525 - protein, 9 aa, synthetic construct
  SITE 1: note = 2,2',2'',2'''-(1,4,7,10-tetraazacyclododecane-1,4,7 ,10-tetrayl)tetraacetic acid--Met-Val-Lys-C(=O)-6-aminohexanoic acid
  SITE 2: note = D-Tryptophan
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
XWNTFGLXW

SEQ ID NO: 526 - protein, 8 aa, synthetic construct
  SITE 1: note = 2,2',2'',2'''-(1,4,7,10-tetraazacyclododecane-1,4,7 ,10-tetrayl)tetraacetic acid--Met-Val-Lys-C(=O)-6-aminohexanoic acid
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
XNTFGLXW

SEQ ID NO: 527 - protein, 8 aa, synthetic construct
  SITE 1: note = 2,2',2'',2'''-(1,4,7,10-tetraazacyclododecane-1,4,7 ,10-tetrayl)tetraacetic acid-D-CsA
  SITE 2: note = D-CsA
  SITE 3: note = 4-(aminomethyl)benzoic acid
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
XXXFGLXW

SEQ ID NO: 528-529 - skipped (2 entries)

SEQ ID NO: 530 - protein, 13 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 4: note = 6-aminohexanoic acid
  SITE 5: note = D-Tyrosine
  SITE 6: note = D-Tryptophan
  SITE 10: note = Azaglycine
  SITE 12: note = 4-amino-1-((3-fluoro-4-methylbenzyl)glycyl)piperidi ne-4-carboxamide
  SITE 13: note = Amidated Tryptophan
XGYXYWNTFGLXW

SEQ ID NO: 531 - protein, 11 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 4: note = D-Tryptophan
  SITE 8: note = Azaglycine
  SITE 10: note = 4-amino-1-((3-fluoro-4-methylbenzyl)glycyl)piperidi ne-4-carboxamide
  SITE 11: note = Amidated Tryptophan
XGYWNTFGLXW

SEQ ID NO: 532 - protein, 10 aa, synthetic construct
  SITE 1: note = 2,2',2'',2'''-(1,4,7,10-tetraazacyclododecane-1,4,7 ,10-tetrayl)tetraacetic acid-Glycine
  SITE 3: note = D-Tryptophan
  SITE 7: note = Azaglycine
  SITE 9: note = 4-amino-1-((3-fluoro-4-methylbenzyl)glycyl)piperidi ne-4-carboxamide
  SITE 10: note = Amidated Tryptophan
GYWNTFGLXW

SEQ ID NO: 533 - protein, 9 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 2: note = D-Tyrosine
  SITE 3: note = D-Tryptophan
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YWNTFGLXW

SEQ ID NO: 534 - protein, 6 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 535 - protein, 8 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 4: note = 3-fluorophenylalanine
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
XNTFGLXW

SEQ ID NO: 536 - protein, 6 aa, synthetic construct
  SITE 1: note = 2,2',2''-(10-(2-(4-(3-carboxypropanamido)piperidin- 1-yl)-2-oxoethyl)-1,4,7,10-tetraazacyclododecane-1,4,7-tri yl)triacetic acid-Phenylalanine
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 537 - protein, 6 aa, synthetic construct
  SITE 1: note = 2,2',2'',2'''-(1,4,7,10-tetraazacyclododecane-1,4,7 ,10-tetrayl)tetraacetic acid-gamma-D-Glutamic acid
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
EFGLXW

SEQ ID NO: 538 - protein, 5 aa, synthetic construct
  SITE 1: note = 2,2',2'',2'''-(1,4,7,10-tetraazacyclododecane-1,4,7 ,10-tetrayl)tetraacetic acid-2,2',2''-(10-(2-(4-carboxypip eridin-1-yl)-2-oxoethyl)-1,4,7,10-tetraazacyclododecane-1, 4,7-triyl)triacetic acid-Phenylalanine
  SITE 2: note = Azaglycine
  SITE 4: note = 4-amino-1-((3-fluoro-4-methylbenzyl)glycyl)piperidi ne-4-carboxamide
  SITE 5: note = Amidated Tryptophan
FGLXW

SEQ ID NO: 539 - protein, 8 aa, synthetic construct
  SITE 1: note = 2,2',2'',2'''-(1,4,7,10-tetraazacyclododecane-1,4,7 ,10-tetrayl)tetraacetic acid-APM
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
XNTFGLXW

SEQ ID NO: 540 - protein, 9 aa, synthetic construct
  SITE 1: note = 2,2',2'',2'''-(1,4,7,10-tetraazacyclododecane-1,4,7 ,10-tetrayl)tetraacetic acid-APM
  SITE 5: note = 3-fluorophenylalanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
XENTFGLXW

SEQ ID NO: 541 - protein, 9 aa, synthetic construct
  SITE 1: note = 2,2',2'',2'''-(1,4,7,10-tetraazacyclododecane-1,4,7 ,10-tetrayl)tetraacetic acid-(1r,4r)-4-(aminomethyl)cyclohexane-1-carboxylic acid
  SITE 5: note = 3-fluorophenylalanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
XENTFGLXW

SEQ ID NO: 542 - protein, 9 aa, synthetic construct
  SITE 1: note = 2,2',2'',2'''-(1,4,7,10-tetraazacyclododecane-1,4,7 ,10-tetrayl)tetraacetic acid-(1r,4r)-4-(aminomethyl)cyclohexane-1-carboxylic acid
  SITE 2: note = D-Glutamic Acid
  SITE 5: note = 3-fluorophenylalanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
XENTFGLXW

SEQ ID NO: 543 - protein, 5 aa, synthetic construct
  SITE 1: note = 2,2',2''-(10-(2-(4-(3-carboxy-N-(2-(2-(2-carboxyeth oxy)ethoxy)ethyl)propanamido)piperidin-1-yl)-2-oxoethyl)-1 ,4,7,10-tetraazacyclododecane-1,4,7-triyl)triacetic acid-Phenylalanine
  SITE 2: note = Azaglycine
  SITE 4: note = 4-amino-1-((3-fluoro-4-methylbenzyl)glycyl)piperidi ne-4-carboxamide
  SITE 5: note = Amidated Tryptophan
FGLXW

SEQ ID NO: 544 - protein, 5 aa, synthetic construct
  SITE 1: note = 2,2',2''-(10-(2-(4-(3-carboxy-N-(2-(2-(2-carboxyeth oxy)ethoxy)ethyl)propanamido)piperidin-1-yl)-2-oxoethyl)-1 ,4,7,10-tetraazacyclododecane-1,4,7-triyl)triacetic acid-Phenylalanine
  SITE 2: note = Azaglycine
  SITE 4: note = 4-amino-1-((3-fluoro-4-methylbenzyl)glycyl)piperidi ne-4-carboxamide
  SITE 5: note = Amidated Tryptophan
FGLXW

SEQ ID NO: 545 - protein, 9 aa, synthetic construct
  SITE 1: note = 2,2',2'',2'''-(1,4,7,10-tetraazacyclododecane-1,4,7 ,10-tetrayl)tetraacetic acid-2-(4-aminopiperidin-1-yl)acetic acid
  SITE 5: note = 3-fluorophenylalanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
XKNTFGLXW

SEQ ID NO: 546 - protein, 7 aa, synthetic construct
  SITE 1: note = 2,2',2'',2'''-(1,4,7,10-tetraazacyclododecane-1,4,7 ,10-tetrayl)tetraacetic acid-gamma-D-Glutamic Acid
  SITE 2: note = (1r,4r)-4-(aminomethyl)cyclohexane-1-carboxylic acid
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
XXFGLXW

SEQ ID NO: 547 - protein, 7 aa, synthetic construct
  SITE 1: note = 2,2',2'',2'''-(1,4,7,10-tetraazacyclododecane-1,4,7 ,10-tetrayl)tetraacetic acid-D-Glutamic Acid
  SITE 2: note = (1r,4r)-4-(aminomethyl)cyclohexane-1-carboxylic acid
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
XXFGLXW

SEQ ID NO: 548 - protein, 7 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid-M-V-4-(aminomethyl)benzoic acid-Met-Val-Lyse-Ada-Asparagine
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
NTFGLXW

SEQ ID NO: 549 - protein, 7 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid-Met-Trp-Lyse-Ada-Asn
  SITE 4: note = Azaglycine
  SITE 6: note = 4-amino-1-((3-fluoro-4-methylbenzyl)glycyl)piperidi ne-4-carboxamide
  SITE 7: note = Amidated Tryptophan
NTFGLXW

SEQ ID NO: 550 - protein, 8 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 4: note = 3-fluorophenylalanine
  SITE 5: note = Azaglycine
  SITE 7: note = 4-amino-1-((3-fluoro-4-methylbenzyl)glycyl)piperidi ne-4-carboxamide
  SITE 8: note = Amidated 3-(4-pyridyl)alanine
XNTFGLXX

SEQ ID NO: 551 - protein, 8 aa, synthetic construct
  SITE 1: note = 2,2',2''-(10-(2-((((1s,4s)-4-carboxycyclohexyl)meth yl)amino)-2-oxoethyl)-1,4,7,10-tetraazacyclododecane-1,4,7 -triyl)triacetic acid coupled-Glu
  SITE 4: note = 3-fluorophenylalanine
  SITE 5: note = Azaglycine
  SITE 7: note = 4-amino-1-((3-fluoro-4-methylbenzyl)glycyl)piperidi ne-4-carboxamide
  SITE 8: note = Amidated 3-(4-pyridyl)alanine
ENTFGLXX

SEQ ID NO: 552 - protein, 8 aa, synthetic construct
  SITE 1: note = 2,2',2'',2'''-(1,4,7,10-tetraazacyclododecane-1,4,7 ,10-tetrayl)tetraacetic acid-Glu
  SITE 4: note = 3-fluorophenylalanine
  SITE 5: note = Azaglycine
  SITE 7: note = 4-amino-1-((3-fluoro-4-methylbenzyl)glycyl)piperidi ne-4-carboxamide
  SITE 8: note = Amidated 3-(4-pyridyl)alanine
ENTFGLXX

SEQ ID NO: 553 - protein, 9 aa, synthetic construct
  SITE 1: note = 2,2',2'',2'''-(1,4,7,10-tetraazacyclododecane-1,4,7 ,10-tetrayl)tetraacetic acid-8-aminoquinoline-4-carboxylic acid
  SITE 5: note = 3-fluorophenylalanine
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
XENTFGLXW

SEQ ID NO: 554 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 3: note = Azaglycine
  SITE 5: note = 4-amino-1-((3-fluoro-4-methylbenzyl)glycyl)piperidi ne-4-carboxamide
  SITE 6: note = Amidated Tyrosine
XFGLXY

SEQ ID NO: 555 - protein, 9 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 4: note = 6-aminohexanoic acid
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
XGYXFGLXW

SEQ ID NO: 556 - protein, 6 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid-Met-Phe-Lyse-C(=O)-6-aminohexanoic acid
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 557 - protein, 6 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid-Met-Trp-Lyse-C(=O)-6-aminohexanoic acid
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 558 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 8: note = norvaline
  SITE 9: note = 4-amino-1-((3-fluoro-4-methylbenzyl)glycyl)piperidi ne-4-carboxamide
  SITE 10: note = Amidated 3-(4-pyridyl)alanine
XYWNTFGXXX

SEQ ID NO: 559 - protein, 10 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 2: note = D-Tyrosine
  SITE 3: note = 3-(2-Naphthyl)alanine
  SITE 7: note = Azaglycine
  SITE 9: note = 4-amino-1-((3-fluoro-4-methylbenzyl)glycyl)piperidi ne-4-carboxamide
  SITE 10: note = Amidated 3-(4-pyridyl)alanine
XYXNTFGLXX

SEQ ID NO: 560 - protein, 9 aa, synthetic construct
  SITE 1: note = C7H15-C(=O)-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-((3-fluoro-4-methylbenzyl)glycyl)piperidi ne-4-carboxamide
  SITE 9: note = Amidated Tryptophan
YKNTFGLXW

SEQ ID NO: 561 - protein, 9 aa, synthetic construct
  SITE 1: note = Me-PEG25-C(=O)-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylpiperidine-4-carboxylic acid
  SITE 9: note = Amidated Tryptophan
YKNTFGLXW

SEQ ID NO: 562 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = 4-benzoylphenylalanine
  SITE 7: note = Azaglycine
  SITE 9: note = 4-amino-1-carbamimidoylpiperidine-4-carboxylic acid
  SITE 10: note = Amidated Tryptophan
XYXNTFGLXW

SEQ ID NO: 563 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = 3-(9-anthryl)-alanine
  SITE 7: note = Azaglycine
  SITE 9: note = 4-amino-1-carbamimidoylpiperidine-4-carboxylic acid
  SITE 10: note = Amidated Tryptophan
XYXNTFGLXW

SEQ ID NO: 564 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-2Nal
  SITE 7: note = Azaglycine
  SITE 9: note = 4-amino-1-carbamimidoylpiperidine-4-carboxylic acid
  SITE 10: note = Amidated Tyrosine
XYXNTFGLXY

SEQ ID NO: 565 - protein, 8 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = D-3-(4-pyridyl)alanine
  SITE 3: note = 6-aminohexanoic acid
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
YXXFGLXW

SEQ ID NO: 566 - protein, 8 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = D-3-(4-pyridyl)alanine
  SITE 3: note = 6-aminohexanoic acid
  SITE 5: note = Azaglycine
  SITE 7: note = 4-amino-1-((3-fluoro-4-methylbenzyl)glycyl)piperidi ne-4-carboxamide
  SITE 8: note = Amidated Tryptophan
YXXFGLXW

SEQ ID NO: 567 - protein, 11 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = 3-(9-anthryl)-alanine
  SITE 5: note = 4-iodophenylalanine
  SITE 6: note = Azaglycine
  SITE 8: note = 4-amino-1-carbamimidoylpiperidine-4-carboxylic acid
  SITE 9: note = Amidated 3-(4-pyridyl)alanine
YXYXNTFGLXX

SEQ ID NO: 568 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 3: note = Azaglycine
  SITE 5: note = 4-amino-1-(N-methylcarbamimidoyl)piperidine-4-carbo xylic acid
  SITE 6: note = Amidated Tryptophan
XFGLXW

SEQ ID NO: 569 - protein, 8 aa, synthetic construct
  SITE 1: note = 3-(2-(2-(3-(p-tolyl)propanamido)ethoxy)ethoxy)propa noic acid coupled to N-term of (S)-2,2',2''-(10-(2-((5-ami no-5-carboxypentyl)amino)-2-oxoethyl)-1,4,7,10-tetraazacyc lododecane-1,4,7-triyl)triacetic acid
  SITE 5: note = Azaglycine
  SITE 7: note = 4-amino-1-carbamimidoylpiperidine-4-carboxylic acid
  SITE 8: note = Amidated 3-(4-pyridyl)alanine
KNTFGLXX

SEQ ID NO: 570 - protein, 8 aa, synthetic construct
  SITE 1: note = 3-(2-(2-(3-(4-iodophenyl)propanamido)ethoxy)ethoxy) propanoic acid coupled to N-term of (S)-2,2',2''-(10-(2-(( 5-amino-5-carboxypentyl)amino)-2-oxoethyl)-1,4,7,10-tetraa zacyclododecane-1,4,7-triyl)triacetic acid
  SITE 5: note = Azaglycine
  SITE 7: note = 4-amino-1-carbamimidoylpiperidine-4-carboxylic acid
  SITE 8: note = Amidated 3-(4-pyridyl)alanine
KNTFGLXX

SEQ ID NO: 571 - protein, 6 aa, synthetic construct
  SITE 1: note = 2,2',2'',2'''-(1,4,7,10-tetraazacyclododecane-1,4,7 ,10-tetrayl)tetraacetic acid-(1r,4r)-4-(aminomethyl)cyclohexane-1-carboxylic acid
  SITE 3: note = Azaglycine
  SITE 5: note = 4-amino-1-((3-fluoro-4-methylbenzyl)glycyl)piperidi ne-4-carboxamide
  SITE 6: note = Amidated Tryptophan
XFGLXW

SEQ ID NO: 572 - protein, 10 aa, synthetic construct
  SITE 1: note = 2,2',2'',2'''-(1,4,7,10-tetraazacyclododecane-1,4,7 ,10-tetrayl)tetraacetic acid-Glycine
  SITE 2: note = D-Tyrosine
  SITE 3: note = D-3-(4-pyridyl)alanine
  SITE 7: note = Azaglycine
  SITE 9: note = 4-amino-1-((3-fluoro-4-methylbenzyl)glycyl)piperidi ne-4-carboxamide
  SITE 10: note = Amidated Tyrosine
GYXNTFGLXY

SEQ ID NO: 573 - protein, 10 aa, synthetic construct
  SITE 1: note = 2,2',2'',2'''-(1,4,7,10-tetraazacyclododecane-1,4,7 ,10-tetrayl)tetraacetic acid-In-Glycine
  SITE 2: note = D-Tyrosine
  SITE 3: note = D-3-(4-pyridyl)alanine
  SITE 7: note = Azaglycine
  SITE 9: note = 4-amino-1-((3-fluoro-4-methylbenzyl)glycyl)piperidi ne-4-carboxamide
  SITE 10: note = Amidated Tyrosine
GYXNTFGLXY

SEQ ID NO: 574 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = 3-(9-anthryl)-alanine
  SITE 7: note = Azaglycine
  SITE 9: note = 4-amino-1-carbamimidoylpiperidine-4-carboxylic acid
  SITE 10: note = Amidated 3-(4-pyridyl)alanine
XYXNTFGLXX

SEQ ID NO: 575 - protein, 9 aa, synthetic construct
  SITE 1: note = 3-(2-(2-(3-(p-tolyl)propanamido)ethoxy)ethoxy)propa noic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylpiperidine-4-carboxylic acid
  SITE 9: note = Amidated Tryptophan
YKNTFGLXW

SEQ ID NO: 576 - protein, 9 aa, synthetic construct
  SITE 1: note = 3-(2-(2-(3-(4-iodophenyl)propanamido)ethoxy)ethoxy) propanoic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylpiperidine-4-carboxylic acid
  SITE 9: note = Amidated Tryptophan
YKNTFGLXW

SEQ ID NO: 577 - protein, 9 aa, synthetic construct
  SITE 1: note = 3-(2-(2-(3-(4-iodophenyl)propanamido)ethoxy)ethoxy) propanoic acid coupled-D-Tyrosine
  SITE 2: note = Indium(III) (S)-2,2',2''-(10-(2-((5-amino-5-carboxy pentyl)amino)-2-oxoethyl)-1,4,7,10-tetraazacyclododecane-1 ,4,7-triyl)triacetate
  SITE 6: note = Azaglycine
  SITE 8: note = 4-amino-1-carbamimidoylpiperidine-4-carboxylic acid
  SITE 9: note = Amidated Tryptophan
YKNTFGLXW

SEQ ID NO: 578 - protein, 9 aa, synthetic construct
  SITE 1: note = 3-(2-(2-methoxyethoxy)ethoxy)propanoic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylpiperidine-4-carboxylic acid
  SITE 9: note = Amidated Tryptophan
YKNTFGLXW

SEQ ID NO: 579 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = biphenylalanine
  SITE 7: note = Azaglycine
  SITE 9: note = 4-amino-1-carbamimidoylpiperidine-4-carboxylic acid
  SITE 10: note = Amidated Tryptophan
XYXNTFGLXW

SEQ ID NO: 580 - protein, 9 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 8: note = 4-amino-1-carbamimidoylpiperidine-4-carboxylic acid
  SITE 9: note = Amidated Phenylalanine
XTNRNFLXF

SEQ ID NO: 581 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-2Nal
  SITE 7: note = Azaglycine
  SITE 9: note = Piperidine-2-carboxylic acid(MeGly)
  SITE 10: note = Amidated Tryptophan
XYXNTFGLXW

SEQ ID NO: 582 - protein, 10 aa, synthetic construct
  SITE 1: note = indium(III) 2,2',2''-(10-(2-((5-carboxypentyl)amino )-2-oxoethyl)-1,4,7,10-tetraazacyclododecane-1,4,7-triyl)t riacetate
  SITE 2: note = D-Tyrosine
  SITE 3: note = D-3-(2-Naphthyl)alanine
  SITE 7: note = Azaglycine
  SITE 9: note = Piperidine-2-carboxylic acid(MeGly)
  SITE 10: note = Amidated Tryptophan
XYXNTFGLXW

SEQ ID NO: 583 - protein, 9 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = 3-(9-anthryl)-alanine
  SITE 6: note = Azaglycine
  SITE 8: note = Piperidine-2-carboxylic acid(MeGly)
  SITE 9: note = Amidated 3-(4-pyridyl)alanine
YXNTFGLXX

SEQ ID NO: 584 - protein, 5 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid-Met-Phe-Lyse-Ada-phenylalanine
  SITE 2: note = Azaglycine
  SITE 4: note = 4-amino-1-((3-fluoro-4-methylbenzyl)glycyl)Piperidi ne-2-carboxylic acideridine-4-carboxamide
  SITE 5: note = Amidated Tryptophan
FGLXW

SEQ ID NO: 585 - skipped

SEQ ID NO: 586 - protein, 8 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated Tyrosine
XNTFGLXY

SEQ ID NO: 587 - protein, 9 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 2: note = D-Glutamic Acid
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tyrosine
XENTFGLXY

SEQ ID NO: 588 - protein, 8 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated 4-cyano phenylalanine
XNTFGLXF

SEQ ID NO: 589 - protein, 9 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated 4-cyano phenylalanine
XENTFGLXF

SEQ ID NO: 590 - protein, 9 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Glutamic Acid
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated 4-cyano phenylalanine
XENTFGLXF

SEQ ID NO: 591 - protein, 7 aa, synthetic construct
  SITE 1: note = 2,2',2''-(10-(2-(((S)-1-((((1s,4R)-4-carboxycyclohe xyl)methyl)amino)-1,5-dioxo-5-(((2S,3R,4R,5R)-2,3,4,5,6-pe ntahydroxyhexyl)amino)pentan-2-yl)amino)-2-oxoethyl)-1,4,7 ,10-tetraazacyclododecane-1,4,7-triyl)triacetic acid-Asn
  SITE 4: note = Azaglycine
  SITE 6: note = 4-amino-1-((3-fluoro-4-methylbenzyl)glycyl)Piperidi ne-2-carboxylic acideridine-4-carboxamide
  SITE 7: note = Amidated Tryptophan
NTFGLXW

SEQ ID NO: 592 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = AzaPhenylalanine
  SITE 3: note = Azaglycine
  SITE 5: note = 4-amino-1-((3-fluoro-4-methylbenzyl)glycyl)Piperidi ne-2-carboxylic acideridine-4-carboxamide
  SITE 6: note = Amidated Tyrosine
XFGLXY

SEQ ID NO: 593 - protein, 7 aa, synthetic construct
  SITE 1: note = 2,2',2''-(10-(2-((((2S,3R,4R,5R,6S)-6-((((1s,4R)-4- carboxycyclohexyl)methyl)carbamoyl)-3,4,5-trihydroxytetrah ydro-2H-pyran-2-yl)methyl)amino)-2-oxoethyl)-1,4,7,10-tetr aazacyclododecane-1,4,7-triyl)triacetic acid coupled to N-term of Asparagine
  SITE 4: note = Azaglycine
  SITE 6: note = 4-amino-1-((3-fluoro-4-methylbenzyl)glycyl)Piperidi ne-2-carboxylic acideridine-4-carboxamide
  SITE 7: note = Amidated Tryptophan
NTFGLXW

SEQ ID NO: 594 - protein, 10 aa, synthetic construct
  SITE 1: note = 2,2',2'',2'''-(1,4,7,10-tetraazacyclododecane-1,4,7 ,10-tetrayl)tetraacetic acid-Glycine
  SITE 3: note = 4-(aminomethyl)benzoic acid
  SITE 7: note = Azaglycine
  SITE 9: note = 4-amino-1-((3-fluoro-4-methylbenzyl)glycyl)Piperidi ne-2-carboxylic acideridine-4-carboxamide
  SITE 10: note = Amidated Tryptophan
GYXNTFGLXW

SEQ ID NO: 595-598 - skipped (4 entries)

SEQ ID NO: 599 - protein, 9 aa, synthetic construct
  SITE 1: note = 2,2',2'',2'''-(1,4,7,10-tetraazacyclododecane-1,4,7 ,10-tetrayl)tetraacetic acid-APM
  SITE 5: note = beta-(2-thienyl)-Alanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
XENTAGLXW

SEQ ID NO: 600 - protein, 9 aa, synthetic construct
  SITE 1: note = 2,2',2'',2'''-(1,4,7,10-tetraazacyclododecane-1,4,7 ,10-tetrayl)tetraacetic acid-APM
  SITE 2: note = O-(dihydroxy(oxo)-l6-phosphaneyl)-L-serine
  SITE 5: note = 3-Fluorophenylalanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
XSNTFGLXW

SEQ ID NO: 601 - protein, 9 aa, synthetic construct
  SITE 1: note = 2,2',2'',2'''-(1,4,7,10-tetraazacyclododecane-1,4,7 ,10-tetrayl)tetraacetic acid-APM
  SITE 2: note = (S)-2-amino-4-(2H-tetrazol-5-yl)butanoic acid
  SITE 5: note = 3-Fluorophenylalanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
XXNTFGLXW

SEQ ID NO: 602 - protein, 9 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = 3-(9-anthryl)-alanine
  SITE 6: note = Azaglycine
  SITE 8: note = 4-amino-1-carbamimidoylPiperidine-2-carboxylic acideridine-4-carboxylic acid
  SITE 9: note = Amidated 3-(4-pyridyl)alanine
YXNTFGLXX

SEQ ID NO: 603 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = 3-(9-anthryl)-alanine
  SITE 7: note = Azaglycine
  SITE 9: note = 4-amino-1-carbamimidoylPiperidine-2-carboxylic acideridine-4-carboxylic acid
  SITE 10: note = Amidated 3-(4-pyridyl)alanine
XYXNTFGLXX

SEQ ID NO: 604 - skipped

SEQ ID NO: 605 - protein, 9 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 2: note = 3-(9-anthryl)-alanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
XXNTFGLXW

SEQ ID NO: 606 - protein, 8 aa, synthetic construct
  SITE 1: note = CH3-(Peg)3-C(=O)-Lys(DOTA)
  SITE 5: note = Azaglycine
  SITE 7: note = 4-amino-1-carbamimidoylPiperidine-2-carboxylic acideridine-4-carboxylic acid
  SITE 8: note = Amidated 3-(4-pyridyl)alanine
KNTFGLXX

SEQ ID NO: 607 - protein, 8 aa, synthetic construct
  SITE 1: note = C7H15-C(=O)-Lyssine(2,2',2'',2'''-(1,4,7,10-tetraaz acyclododecane-1,4,7,10-tetrayl)tetraacetic acid)
  SITE 5: note = Azaglycine
  SITE 7: note = 4-amino-1-carbamimidoylPiperidine-2-carboxylic acideridine-4-carboxylic acid
  SITE 8: note = Amidated 3-(4-pyridyl)alanine
KNTFGLXX

SEQ ID NO: 608 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-4-iodophenylalanine
  SITE 3: note = D-Tryptophan
  SITE 7: note = Azaglycine
  SITE 9: note = 4-amino-1-(N-methylcarbamimidoyl)Piperidine-2-carbo xylic acideridine-4-carboxylic acid
  SITE 10: note = Amidated Tryptophan
XFWNTFGLXW

SEQ ID NO: 609 - skipped

SEQ ID NO: 610 - protein, 10 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 7: note = Azaglycine
  SITE 9: note = 4-amino-1-(N-methylcarbamimidoyl)Piperidine-2-carbo xylic acideridine-4-carboxylic acid
  SITE 10: note = Amidated Tryptophan
XKWNTFGLXW

SEQ ID NO: 611 - protein, 8 aa, synthetic construct
  SITE 1: note = 2,2',2'',2'''-(1,4,7,10-tetraazacyclododecane-1,4,7 ,10-tetrayl)tetraacetic acid-D-bGlu
  SITE 4: note = 3-fluorophenylalanine
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated 3-(4-pyridyl)alanine
ENTFGLXX

SEQ ID NO: 612 - protein, 9 aa, synthetic construct
  SITE 1: note = 2,2',2'',2'''-(1,4,7,10-tetraazacyclododecane-1,4,7 ,10-tetrayl)tetraacetic acid-(1r,4r)-4-(aminomethyl)cyclohexane-1-carboxylic acid
  SITE 2: note = D-3-aminopentanedioic acid
  SITE 5: note = 3-fluorophenylalanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated 3-(4-pyridyl)alanine
XENTFGLXX

SEQ ID NO: 613 - protein, 9 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 2: note = D-3-aminopentanedioic acid
  SITE 5: note = 3-fluorophenylalanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated 3-(4-pyridyl)alanine
XENTFGLXX

SEQ ID NO: 614 - protein, 7 aa, synthetic construct
  SITE 1: note = 2,2',2'',2'''-(1,4,7,10-tetraazacyclododecane-1,4,7 ,10-tetrayl)tetraacetic acid-APM
  SITE 4: note = Azaglycine
  SITE 6: note = 4-amino-1-((3-fluoro-4-methylbenzyl)glycyl)Piperidi ne-2-carboxylic acideridine-4-carboxamide
  SITE 7: note = Amidated Tryptophan
XTFGLXW

SEQ ID NO: 615 - protein, 8 aa, synthetic construct
  SITE 1: note = 2,2',2'',2'''-(1,4,7,10-tetraazacyclododecane-1,4,7 ,10-tetrayl)tetraacetic acid-APM
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated Tryptophan
XETFGLXW

SEQ ID NO: 616 - protein, 8 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 5: note = Azaglycine
  SITE 7: note = 4-amino-1-((3-fluoro-4-methylbenzyl)glycyl)piperidi ne-4-carboxamide(4-OMe)
  SITE 8: note = Amidated Phenylalanine
XNTFGLXF

SEQ ID NO: 617 - protein, 9 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 6: note = Azaglycine
  SITE 8: note = Apc(4-OMe)
  SITE 9: note = Amidated Phenylalanine
XENTFGLXF

SEQ ID NO: 618 - protein, 8 aa, synthetic construct
  SITE 1: note = 2,2',2'',2'''-(1,4,7,10-tetraazacyclododecane-1,4,7 ,10-tetrayl)tetraacetic acid-Glu
  SITE 5: note = Azaglycine
  SITE 7: note = Apc(4-OMe)
  SITE 8: note = Amidated Phenylalanine
ENTFGLXF

SEQ ID NO: 619 - protein, 9 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
XENTFGLXW

SEQ ID NO: 620 - protein, 9 aa, synthetic construct
  SITE 1: note = 2,2',2'',2'''-(1,4,7,10-tetraazacyclododecane-1,4,7 ,10-tetrayl)tetraacetic acid-Glu
  SITE 5: note = Azaglycine
  SITE 6: note = D-Leucine
  SITE 7: note = Apc(4-OMe)
  SITE 8: note = Amidated Phenylalanine
XENTFGLXF

SEQ ID NO: 621 - protein, 8 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 5: note = 2,2',2'',2'''-(1,4,7,10-tetraazacyclododecane-1,4,7 ,10-tetrayl)tetraacetic acid-4-(aminomethyl)benzoic acid
  SITE 7: note = 4-amino-1-((3-fluoro-4-methylbenzyl)glycyl)Piperidi ne-2-carboxylic acideridine-4-carboxamide
  SITE 8: note = Amidated 4-cyano phenylalanine
XNTFGLXF

SEQ ID NO: 622 - protein, 9 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated 4-cyano phenylalanine
XENTFGLXF

SEQ ID NO: 623 - protein, 9 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 2: note = D-Glutamic Acid
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated 4-cyano phenylalanine
XENTFGLXF

SEQ ID NO: 624 - protein, 8 aa, synthetic construct
  SITE 1: note = 2,2',2'',2'''-(1,4,7,10-tetraazacyclododecane-1,4,7 ,10-tetrayl)tetraacetic acid-4-oxo-4-(Piperidine-2-carboxylic acideridin-4-ylamino)butanoic acid
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated Tryptophan
XNTFGLXW

SEQ ID NO: 625 - protein, 6 aa, synthetic construct
  SITE 1: note = 2,2',2'',2'''-(1,4,7,10-tetraazacyclododecane-1,4,7 ,10-tetrayl)tetraacetic acid-APM
  SITE 3: note = Azaglycine
  SITE 5: note = 4-amino-1-((3-fluoro-4-methylbenzyl)glycyl)Piperidi ne-2-carboxylic acideridine-4-carboxamide
  SITE 6: note = Amidated 4-cyano phenylalanine
XFGLXF

SEQ ID NO: 626 - protein, 6 aa, synthetic construct
  SITE 1: note = 2,2',2'',2'''-(1,4,7,10-tetraazacyclododecane-1,4,7 ,10-tetrayl)tetraacetic acid-APM
  SITE 3: note = Azaglycine
  SITE 5: note = 4-amino-1-((3-fluoro-4-methylbenzyl)glycyl)Piperidi ne-2-carboxylic acideridine-4-carboxamide
  SITE 6: note = Amidated Phenylalanine
XFGLXF

SEQ ID NO: 627 - skipped

SEQ ID NO: 628 - protein, 6 aa, synthetic construct
  SITE 1: note = 2,2',2'',2'''-(1,4,7,10-tetraazacyclododecane-1,4,7 ,10-tetrayl)tetraacetic acid-APM
  SITE 3: note = Azaglycine
  SITE 5: note = 4-amino-1-((3-fluoro-4-methylbenzyl)glycyl)Piperidi ne-2-carboxylic acideridine-4-carboxamide
  SITE 6: note = Amidated Tyrosine
XFGLXY

SEQ ID NO: 629 - protein, 6 aa, synthetic construct
  SITE 1: note = 2,2',2'',2'''-(1,4,7,10-tetraazacyclododecane-1,4,7 ,10-tetrayl)tetraacetic acid-APM
  SITE 3: note = Azaglycine
  SITE 5: note = 4-amino-1-((3-fluoro-4-methylbenzyl)glycyl)Piperidi ne-2-carboxylic acideridine-4-carboxamide
  SITE 6: note = Amidated Histidine
XFGLXH

SEQ ID NO: 630 - protein, 8 aa, synthetic construct
  SITE 1: note = 2,2',2'',2'''-(1,4,7,10-tetraazacyclododecane-1,4,7 ,10-tetrayl)tetraacetic acid-APM
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated Tyrosine
XNTFGLXY

SEQ ID NO: 631 - protein, 7 aa, synthetic construct
  SITE 1: note = 2,2',2'',2'''-(1,4,7,10-tetraazacyclododecane-1,4,7 ,10-tetrayl)tetraacetic acid-APM
  SITE 4: note = Azaglycine
  SITE 6: note = 4-amino-1-((3-fluoro-4-methylbenzyl)glycyl)Piperidi ne-2-carboxylic acideridine-4-carboxamide
  SITE 7: note = Amidated Tyrosine
XTFGLXY

SEQ ID NO: 632 - protein, 7 aa, synthetic construct
  SITE 1: note = 3-(2-(2-hydroxyethoxy)ethoxy)propanoic acid-Lys-(DOTA)
  SITE 4: note = Azaglycine
  SITE 6: note = 4-amino-1-((3-fluoro-4-methylbenzyl)glycyl)Piperidi ne-2-carboxylic acideridine-4-carboxamide
  SITE 7: note = Amidated 3-(4-pyridyl)alanine
KTFGLXX

SEQ ID NO: 633 - protein, 8 aa, synthetic construct
  SITE 1: note = 3-(2-(2-hydroxyethoxy)ethoxy)propanoic acid-Lys(DOTA)
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated 3-(4-pyridyl)alanine
KETFGLXX

SEQ ID NO: 634 - protein, 8 aa, synthetic construct
  SITE 1: note = 3-(2-(2-hydroxyethoxy)ethoxy)propanoic acid-Lys(DOTA)
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated 3-(4-pyridyl)alanine
KNTFGLXX

SEQ ID NO: 635 - protein, 8 aa, synthetic construct
  SITE 1: note = 3-(2-(2-hydroxyethoxy)ethoxy)propanoic acid-D-Lys(DOTA)
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated 3-(4-pyridyl)alanine
KNTFGLXX

SEQ ID NO: 636 - protein, 8 aa, synthetic construct
  SITE 1: note = 3-(2-(2-methoxyethoxy)ethoxy)propanoic acid-Lys(DOTA)
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated 3-(4-pyridyl)alanine
KNTFGLXX

SEQ ID NO: 637 - protein, 8 aa, synthetic construct
  SITE 1: note = 1-hydroxy-3,6,9,12,15,18,21,24-octaoxaheptacosan-27 -oic acid-Lys(DOTA)
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated 3-(4-pyridyl)alanine
KNTFGLXX

SEQ ID NO: 638 - protein, 8 aa, synthetic construct
  SITE 1: note = 2,2',2'',2'''-(1,4,7,10-tetraazacyclododecane-1,4,7 ,10-tetrayl)tetraacetic acid-APM
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Tyr(3-NO2)-NH2
XNTFGLXY

SEQ ID NO: 639 - protein, 8 aa, synthetic construct
  SITE 1: note = 2,2',2'',2'''-(1,4,7,10-tetraazacyclododecane-1,4,7 ,10-tetrayl)tetraacetic acid-APM
  SITE 4: note = 3-(4-pyridyl)alanine
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated 3-(4-pyridyl)alanine
XNTXGLXX

SEQ ID NO: 640 - protein, 8 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 4: note = 3-(4-pyridyl)alanine
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated 3-(4-pyridyl)alanine
XNTXGLXX

SEQ ID NO: 641 - protein, 6 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 3: note = Azaglycine
  SITE 4: note = D-Leucine
  SITE 5: note = 4-amino-1-((3-fluoro-4-methylbenzyl)glycyl)Piperidi ne-2-carboxylic acideridine-4-carboxamide
  SITE 6: note = Amidated Tryptophan
XFGLXW

SEQ ID NO: 642 - skipped

SEQ ID NO: 643 - protein, 8 aa, synthetic construct
  SITE 1: note = 2,2',2'',2'''-(1,4,7,10-tetraazacyclododecane-1,4,7 ,10-tetrayl)tetraacetic acid-8-aminoquinoline-3-carboxylic acid
  SITE 5: note = Azaglycine
  SITE 7: note = 4-amino-1-carbamimidoylPiperidine-2-carboxylic acideridine-4-carboxylic acid
  SITE 8: note = Amidated Tryptophan
XNTFGLXW

SEQ ID NO: 644 - skipped

SEQ ID NO: 645 - protein, 8 aa, synthetic construct
  SITE 1: note = 3-(2-(2-methoxyethoxy)ethoxy)propanoic acid-Lys(DOTA)
  SITE 5: note = Azaglycine
  SITE 7: note = 4-amino-1-carbamimidoylPiperidine-2-carboxylic acideridine-4-carboxylic acid
  SITE 8: note = Amidated Tryptophan
KNTFGLXW

SEQ ID NO: 646 - skipped

SEQ ID NO: 647 - protein, 9 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = 3-(2-Naphthyl)alanine or
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YXNTFGLXW

SEQ ID NO: 648 - protein, 6 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 3: note = Azaglycine
  SITE 5: note = 4-amino-1-((3-fluoro-4-methylbenzyl)glycyl)Piperidi ne-2-carboxylic acideridine-4-carboxamide
  SITE 6: note = Amidated Tryptophan
XYGLXW

SEQ ID NO: 649 - skipped

SEQ ID NO: 650 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = 4-amino-1-carbamimidoylPiperidine-2-carboxylic acideridine-4-carboxylic acid
  SITE 10: note = Amidated Tryptophan
XYXNTFGLXW

SEQ ID NO: 651 - protein, 10 aa, synthetic construct
  SITE 1: note = indium(III) 2,2',2''-(10-(2-((5-carboxypentyl)amino )-2-oxoethyl)-1,4,7,10-tetraazacyclododecane-1,4,7-triyl)t riacetate
  SITE 2: note = D-Tyrosine
  SITE 3: note = D-3-(2-Naphthyl)alanine
  SITE 7: note = Azaglycine
  SITE 9: note = 4-amino-1-carbamimidoylPiperidine-2-carboxylic acideridine-4-carboxylic acid
  SITE 10: note = Amidated Tryptophan
XYXNTFGLXW

SEQ ID NO: 652 - protein, 11 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = 4-(aminomethyl)benzoic acid
  SITE 3: note = D-Tyrosine
  SITE 4: note = D-Tryptophan
  SITE 8: note = Azaglycine
  SITE 10: note = 4-amino-1-carbamimidoylPiperidine-2-carboxylic acideridine-4-carboxylic acid
  SITE 11: note = Amidated Tryptophan
XXYWNTFGLXW

SEQ ID NO: 653 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 6: note = biphenylalanine
  SITE 7: note = Azaglycine
  SITE 9: note = 4-amino-1-carbamimidoylPiperidine-2-carboxylic acideridine-4-carboxylic acid
  SITE 10: note = Amidated Tryptophan
XYWNTXGLXW

SEQ ID NO: 654 - protein, 8 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 5: note = Azaglycine
  SITE 7: note = 4-amino-1-carbamimidoylPiperidine-2-carboxylic acideridine-4-carboxylic acid
  SITE 8: note = Amidated 3-(4-pyridyl)alanine
XNTFGLXX

SEQ ID NO: 655 - protein, 9 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = 3-(4-pyridyl)alanine
  SITE 6: note = Azaglycine
  SITE 8: note = 4-amino-1-carbamimidoylPiperidine-2-carboxylic acideridine-4-carboxylic acid
  SITE 9: note = Amidated 3-(4-pyridyl)alanine
YXNTFGLXX

SEQ ID NO: 656 - skipped

SEQ ID NO: 657 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = 3-(2-Naphthyl)alanine or
  SITE 7: note = Azaglycine
  SITE 9: note = 4-amino-1-carbamimidoylPiperidine-2-carboxylic acideridine-4-carboxylic acid
  SITE 10: note = Amidated 3-(4-pyridyl)alanine
XYXNTFGLXX

SEQ ID NO: 658 - protein, 10 aa, synthetic construct
  SITE 1: note = (DOTA)-6-aminohexanoic acid
  SITE 2: note = D-Tyrosine
  SITE 3: note = 3-(2-Naphthyl)alanine or
  SITE 7: note = Azaglycine
  SITE 9: note = 4-amino-1-carbamimidoylPiperidine-2-carboxylic acideridine-4-carboxylic acid
  SITE 10: note = Amidated Tyrosine
XYXNTFGLXY

SEQ ID NO: 659 - protein, 10 aa, synthetic construct
  SITE 1: note = (DOTA)-6-aminohexanoic acid
  SITE 2: note = D-Tyrosine
  SITE 3: note = 3-(2-Naphthyl)alanine or
  SITE 7: note = Azaglycine
  SITE 9: note = 4-amino-1-carbamimidoylPiperidine-2-carboxylic acideridine-4-carboxylic acid
  SITE 10: note = Amidated Phenylalanine
XYXNTFGLXF

SEQ ID NO: 660 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = 3-(2-Naphthyl)alanine or
  SITE 7: note = Azaglycine
  SITE 9: note = 4-amino-1-carbamimidoylPiperidine-2-carboxylic acideridine-4-carboxylic acid
  SITE 10: note = Amidated 4-cyano phenylalanine
XYXNTFGLXF

SEQ ID NO: 661 - protein, 9 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = 2-(2-aminoethoxy)-[1,1'-biphenyl]-4-carboxylic acid
  SITE 8: note = 4-amino-1-((3-fluoro-4-methylbenzyl)glycyl)Piperidi ne-2-carboxylic acideridine-4-carboxamide
  SITE 9: note = Amidated Tryptophan
XYWNTFXXW

SEQ ID NO: 662 - skipped

SEQ ID NO: 663 - protein, 9 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = D-3-(4-pyridyl)alanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated 3-(4-pyridyl)alanine
YXNTFGLXX

SEQ ID NO: 664-666 - skipped (3 entries)

SEQ ID NO: 667 - protein, 8 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = 3-(2-Naphthyl)alanine or
  SITE 3: note = 6-aminohexanoic acid
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated Tryptophan
YXXFGLXW

SEQ ID NO: 668 - protein, 8 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = 3-(2-Naphthyl)alanine or
  SITE 3: note = 6-aminohexanoic acid
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated 3-(4-pyridyl)alanine
YXXFGLXX

SEQ ID NO: 669 - protein, 8 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = 3-(2-Naphthyl)alanine or
  SITE 3: note = ahx
  SITE 5: note = Azaglycine
  SITE 7: note = 4-amino-1-carbamimidoylPiperidine-2-carboxylic acideridine-4-carboxylic acid
  SITE 8: note = Amidated Tryptophan
YXXFGLXW

SEQ ID NO: 670 - protein, 8 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = 3-(2-Naphthyl)alanine or
  SITE 3: note = 4-(aminomethyl)benzoic acid
  SITE 5: note = Azaglycine
  SITE 7: note = 4-amino-1-carbamimidoylPiperidine-2-carboxylic acideridine-4-carboxylic acid
  SITE 8: note = Amidated Phenylalanine
YXXFGLXF

SEQ ID NO: 671 - protein, 8 aa, synthetic construct
  SITE 1: note = 2,2',2'',2'''-(1,4,7,10-tetraazacyclododecane-1,4,7 ,10-tetrayl)tetraacetic acid-D-Tyrosine
  SITE 2: note = 3-(2-Naphthyl)alanine or
  SITE 3: note = 6-aminohexanoic acid
  SITE 5: note = Azaglycine
  SITE 7: note = 4-amino-1-carbamimidoylPiperidine-2-carboxylic acideridine-4-carboxylic acid
  SITE 8: note = Amidated Phenylalanine
YXXFGLXF

SEQ ID NO: 672 - protein, 9 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tryptophan
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated 3-(4-pyridyl)alanine
XWNTFGLXX

SEQ ID NO: 673 - protein, 9 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated 3-(4-pyridyl)alanine
XYNTFGLXX

SEQ ID NO: 674 - protein, 9 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-3-(2-Naphthyl)alanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated 3-(4-pyridyl)alanine
XXNTFGLXX

SEQ ID NO: 675 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = Apc(OMe)
  SITE 10: note = Amidated 3-(4-pyridyl)alanine
XYWNTFGLXX

SEQ ID NO: 676 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(4-pyridyl)alanine
  SITE 7: note = Azaglycine
  SITE 9: note = 4-amino-1-((3-fluoro-4-methylbenzyl)glycyl)Piperidi ne-2-carboxylic acideridine-4-carboxamide
  SITE 10: note = Amidated 3-(4-pyridyl)alanine
XYXNTFGLXX

SEQ ID NO: 677 - protein, 9 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-3-(4-pyridyl)alanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated 3-(4-pyridyl)alanine
XXNTFGLXX

SEQ ID NO: 678 - protein, 9 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-3-(4-pyridyl)alanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
XXNTFGLXW

SEQ ID NO: 679-680 - skipped (2 entries)

SEQ ID NO: 681 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(4-pyridyl)alanine
  SITE 7: note = Azaglycine
  SITE 9: note = Apc(OMe)
  SITE 10: note = Amidated 3-(4-pyridyl)alanine
XYXNTFGLXX

SEQ ID NO: 682 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = 4-amino-1-carbamimidoylPiperidine-2-carboxylic acideridine-4-carboxylic acid
  SITE 10: note = Amidated Phenylalanine
XYXNTFGLXF

SEQ ID NO: 683 - protein, 9 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-3-(2-Naphthyl)alanine
  SITE 6: note = Azaglycine
  SITE 8: note = 4-amino-1-carbamimidoylPiperidine-2-carboxylic acideridine-4-carboxylic acid
  SITE 9: note = Amidated Phenylalanine
XXNTFGLXF

SEQ ID NO: 684 - protein, 8 aa, synthetic construct
  SITE 1: note = 2,2',2'',2'''-(1,4,7,10-tetraazacyclododecane-1,4,7 ,10-tetrayl)tetraacetic acid-D-3-(2-Naphthyl)alanine
  SITE 5: note = Azaglycine
  SITE 7: note = 4-amino-1-carbamimidoylPiperidine-2-carboxylic acideridine-4-carboxylic acid
  SITE 8: note = Amidated Phenylalanine
XNTFGLXF

SEQ ID NO: 685 - skipped

SEQ ID NO: 686 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = 4-amino-1-((3-fluoro-4-methylbenzyl)glycyl)Piperidi ne-2-carboxylic acideridine-4-carboxamide
  SITE 10: note = Amidated Tryptophan
XYXNTFGLXW

SEQ ID NO: 687 - protein, 8 aa, synthetic construct
  SITE 1: note = 2,2',2'',2'''-(1,4,7,10-tetraazacyclododecane-1,4,7 ,10-tetrayl)tetraacetic acid-Glycine
  SITE 3: note = 6-aminohexanoic acid
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated Tryptophan
GYXFGLXW

SEQ ID NO: 688 - protein, 9 aa, synthetic construct
  SITE 1: note = 2,2',2'',2'''-(1,4,7,10-tetraazacyclododecane-1,4,7 ,10-tetrayl)tetraacetic acid-Glycine
  SITE 2: note = D-Tyrosine
  SITE 3: note = 3-(2-Naphthyl)alanine or
  SITE 4: note = 6-aminohexanoic acid
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
GYXXFGLXW

SEQ ID NO: 689 - protein, 9 aa, synthetic construct
  SITE 1: note = 2,2',2'',2'''-(1,4,7,10-tetraazacyclododecane-1,4,7 ,10-tetrayl)tetraacetic acid-Glycine
  SITE 3: note = 3-(2-Naphthyl)alanine or
  SITE 4: note = 6-aminohexanoic acid
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
GYXXFGLXW

SEQ ID NO: 690 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-hydroxyproline
  SITE 7: note = Azaglycine
  SITE 9: note = 4-amino-1-((3-fluoro-4-methylbenzyl)glycyl)Piperidi ne-2-carboxylic acideridine-4-carboxamide
  SITE 10: note = Amidated Tryptophan
XYXNTFGLXW

SEQ ID NO: 691 - protein, 11 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = N6-(4-(p-tolyl)butanoyl)-D-lysine
  SITE 3: note = D-Tyrosine
  SITE 4: note = D-3-(2-Naphthyl)alanine
  SITE 8: note = Azaglycine
  SITE 10: note = 4-amino-1-carbamimidoylPiperidine-2-carboxylic acideridine-4-carboxylic acid
  SITE 11: note = Amidated Phenylalanine
XXYXNTFGLXF

SEQ ID NO: 692 - protein, 11 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = N6-(4-(4-iodophenyl)butanoyl)-D-lysine
  SITE 3: note = D-Tyrosine
  SITE 4: note = D-3-(2-Naphthyl)alanine
  SITE 8: note = Azaglycine
  SITE 10: note = 4-amino-1-carbamimidoylPiperidine-2-carboxylic acideridine-4-carboxylic acid
  SITE 11: note = Amidated Phenylalanine
XXYXNTFGLXF

SEQ ID NO: 693 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = hydroxyproline
  SITE 7: note = Azaglycine
  SITE 9: note = 4-amino-1-carbamimidoylPiperidine-2-carboxylic acideridine-4-carboxylic acid
  SITE 10: note = Amidated Tryptophan
XYXNTFGLXW

SEQ ID NO: 694 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-hydroxyproline
  SITE 7: note = Azaglycine
  SITE 9: note = 4-amino-1-carbamimidoylPiperidine-2-carboxylic acideridine-4-carboxylic acid
  SITE 10: note = Amidated Tryptophan
XYXNTFGLXW

SEQ ID NO: 695 - protein, 8 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 4: note = cyclohexylalanine
  SITE 5: note = Azaglycine
  SITE 7: note = 4-amino-1-((3-fluoro-4-methylbenzyl)glycyl)Piperidi ne-2-carboxylic acideridine-4-carboxamide
  SITE 8: note = Amidated Tryptophan
XNTXGLXW

SEQ ID NO: 696 - protein, 9 aa, synthetic construct
  SITE 1: note = (2,2',2'',2'''-(1,4,7,10-tetraazacyclododecane-1,4, 7,10-tetrayl)tetraacetic acid)-6-aminohexanoic acid
  SITE 2: note = D-3-(4-pyridyl)alanine
  SITE 5: note = cyclohexylalanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
XXNTXGLXW

SEQ ID NO: 697 - protein, 9 aa, synthetic construct
  SITE 1: note = (2,2',2'',2'''-(1,4,7,10-tetraazacyclododecane-1,4, 7,10-tetrayl)tetraacetic acid)-D-Tyrosine
  SITE 2: note = D-3-(4-pyridyl)alanine
  SITE 5: note = cyclohexylalanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
YXNTXGLXW

SEQ ID NO: 698 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(4-pyridyl)alanine
  SITE 6: note = cyclohexylalanine
  SITE 7: note = Azaglycine
  SITE 9: note = 4-amino-1-((3-fluoro-4-methylbenzyl)glycyl)Piperidi ne-2-carboxylic acideridine-4-carboxamide
  SITE 10: note = Amidated Tryptophan
XYXNTXGLXW

SEQ ID NO: 699 - protein, 10 aa, synthetic construct
  SITE 1: note = (DOTA)-6-aminohexanoic acid
  SITE 2: note = D-Tyrosine
  SITE 3: note = D-3-(2-Naphthyl)alanine
  SITE 6: note = cyclohexylalanine
  SITE 7: note = Azaglycine
  SITE 9: note = 4-amino-1-carbamimidoylPiperidine-2-carboxylic acideridine-4-carboxylic acid
  SITE 10: note = Amidated Tryptophan
XYXNTXGLXW

SEQ ID NO: 700 - protein, 9 aa, synthetic construct
  SITE 1: note = (DOTA)-D-Tyrosine
  SITE 2: note = D-3-(2-Naphthyl)alanine
  SITE 5: note = cyclohexylalanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
YXNTXGLXW

SEQ ID NO: 701 - protein, 10 aa, synthetic construct
  SITE 1: note = (DOTA)-6-aminohexanoic acid
  SITE 2: note = D-Tyrosine
  SITE 3: note = D-3-(2-Naphthyl)alanine
  SITE 7: note = Azaglycine
  SITE 9: note = 4-amino-1-(N-methylcarbamimidoyl)Piperidine-2-carbo xylic acideridine-4-carboxylic acid
  SITE 10: note = Amidated D-Tryptophan
XYXNTFGLXW

SEQ ID NO: 702 - protein, 10 aa, synthetic construct
  SITE 1: note = 3-(4-iodophenyl)propanoic acid-gamma-D-Glu
  SITE 2: note = D-Tyrosine
  SITE 3: note = Lysine(1,4,7,10-tetraazacyclododecane-1,4,7,10-tetr aacetic acid)
  SITE 7: note = Azaglycine
  SITE 9: note = 4-amino-1-carbamimidoylPiperidine-2-carboxylic acideridine-4-carboxylic acid
  SITE 10: note = Amidated Tryptophan
EYKNTFGLXW

SEQ ID NO: 703 - protein, 9 aa, synthetic construct
  SITE 1: note = 3-(4-iodophenyl)propanoic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YKNTFGLXW

SEQ ID NO: 704 - protein, 9 aa, synthetic construct
  SITE 1: note = 3-(2-(2-(3-(p-tolyl)propanamido)ethoxy)ethoxy)propa noic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylPiperidine-2-carboxylic acideridine-4-carboxylic acid
  SITE 9: note = Amidated Phenylalanine
YKNTFGLXF

SEQ ID NO: 705 - protein, 9 aa, synthetic construct
  SITE 1: note = 3-(2-(2-(3-(4-iodophenyl)propanamido)ethoxy)ethoxy) propanoic-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylPiperidine-2-carboxylic acideridine-4-carboxylic acid
  SITE 9: note = Amidated Phenylalanine
YKNTFGLXF

SEQ ID NO: 706 - protein, 9 aa, synthetic construct
  SITE 1: note = 3-(2-(2-(3-(4-iodophenyl)propanamido)ethoxy)ethoxy) propanoic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylPiperidine-2-carboxylic acideridine-4-carboxylic acid
  SITE 9: note = Amidated Tyrosine
YKNTFGLXY

SEQ ID NO: 707 - protein, 9 aa, synthetic construct
  SITE 1: note = 3-(2-(2-(3-(4-iodophenyl)propanamido)ethoxy)ethoxy) propanoic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylPiperidine-2-carboxylic acideridine-4-carboxylic acid
  SITE 9: note = Amidated 4-cyano phenylalanine
YKNTFGLXF

SEQ ID NO: 708 - protein, 9 aa, synthetic construct
  SITE 1: note = 3-(2-(2-(3-(p-tolyl)propanamido)ethoxy)ethoxy)propa noic acid-D-Tyrosine
  SITE 2: note = Lysine(1,4,7,10-tetraazacyclododecane-1,4,7,10-tetr aacetic acid)
  SITE 5: note = cyclohexylalanine
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YKNTXGLXW

SEQ ID NO: 709 - protein, 9 aa, synthetic construct
  SITE 1: note = 3-(2-(2-(3-(4-iodophenyl)propanamido)ethoxy)ethoxy) propanoic acid-D-Tyrosine
  SITE 2: note = Lysine(1,4,7,10-tetraazacyclododecane-1,4,7,10-tetr aacetic acid)
  SITE 5: note = cyclohexylalanine
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YKNTXGLXW

SEQ ID NO: 710 - protein, 9 aa, synthetic construct
  SITE 1: note = palmitic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YKNTFGLXW

SEQ ID NO: 711 - protein, 9 aa, synthetic construct
  SITE 1: note = dodecanoic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YKNTFGLXW

SEQ ID NO: 712 - protein, 9 aa, synthetic construct
  SITE 1: note = palmitic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylPiperidine-2-carboxylic acideridine-4-carboxylic acid
  SITE 9: note = Amidated Tyrosine
YKNTFGLXY

SEQ ID NO: 713 - protein, 9 aa, synthetic construct
  SITE 1: note = dodecanoic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylPiperidine-2-carboxylic acideridine-4-carboxylic acid
  SITE 9: note = Amidated Tyrosine
YKNTFGLXY

SEQ ID NO: 714 - protein, 9 aa, synthetic construct
  SITE 1: note = octanoic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylPiperidine-2-carboxylic acideridine-4-carboxylic acid
  SITE 9: note = Amidated Tyrosine
YKNTFGLXY

SEQ ID NO: 715 - protein, 9 aa, synthetic construct
  SITE 1: note = 3-(2-(2-(3-(p-tolyl)propanamido)ethoxy)ethoxy)propa noic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylPiperidine-2-carboxylic acideridine-4-carboxylic acid
  SITE 9: note = Amidated 4-cyano phenylalanine
YKNTFGLXF

SEQ ID NO: 716 - protein, 9 aa, synthetic construct
  SITE 1: note = 3-(2-(2-(3-(4-iodophenyl)propanamido)ethoxy)ethoxy) propanoic acid-D-Tyrosine
  SITE 2: note = D-Lysine(1,4,7,10-tetraazacyclododecane-1,4,7,10-te traacetic acid)
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YKNTFGLXW

SEQ ID NO: 717 - protein, 11 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = N6-(4-(p-tolyl)butanoyl)-D-lysine
  SITE 3: note = D-Tyrosine
  SITE 4: note = D-3-(2-Naphthyl)alanine
  SITE 8: note = Azaglycine
  SITE 10: note = 4-amino-1-carbamimidoylPiperidine-2-carboxylic acideridine-4-carboxylic acid
  SITE 11: note = Amidated Tryptophan
XKYXNTFGLXW

SEQ ID NO: 718 - protein, 11 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = N6-(4-(4-iodophenyl)butanoyl)-D-lysine
  SITE 3: note = D-Tyrosine
  SITE 4: note = D-2-Nal
  SITE 8: note = Azaglycine
  SITE 10: note = 4-amino-1-carbamimidoylPiperidine-2-carboxylic acideridine-4-carboxylic acid
  SITE 11: note = Amidated Tryptophan
XKYXNTFGLXW

SEQ ID NO: 719 - protein, 12 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = N6-(4-(4-iodophenyl)butanoyl)-D-lysine
  SITE 3: note = ahx
  SITE 4: note = D-Tyrosine
  SITE 5: note = D-3-(2-Naphthyl)alanine
  SITE 9: note = Azaglycine
  SITE 11: note = 4-amino-1-carbamimidoylPiperidine-2-carboxylic acideridine-4-carboxylic acid
  SITE 12: note = Amidated Tryptophan
XKXYXNTFGLXW

SEQ ID NO: 720 - protein, 9 aa, synthetic construct
  SITE 1: note = 3-(2-(2-(3-(p-tolyl)propanamido)ethoxy)ethoxy)propa noic acid-D-Tyrosine
  SITE 2: note = D-Lysine(1,4,7,10-tetraazacyclododecane-1,4,7,10-te traacetic acid)
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YKNTFGLXW

SEQ ID NO: 721 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = Piperidine-2-carboxylic acid
  SITE 10: note = Amidated D-Tryptophan
XYXNTFGLXW

SEQ ID NO: 722 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = 4-amino-1-(methyl-D-alanyl)Piperidine-2-carboxylic acideridine-4-carboxylic acid
  SITE 10: note = Amidated Tryptophan
XYXNTFGLXW

SEQ ID NO: 723 - protein, 9 aa, synthetic construct
  SITE 1: note = 1-(3-fluoro-4-methylphenyl)-4-oxo-8,11-dioxa-2,5-di azatetradecan-14-oic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YKNTFGLXW

SEQ ID NO: 724 - protein, 10 aa, synthetic construct
  SITE 1: note = (3-fluoro-4-methylbenzyl)glycine
  SITE 2: note = D-Tyrosine
  SITE 3: note = Lysine(1,4,7,10-tetraazacyclododecane-1,4,7,10-tetr aacetic acid)
  SITE 7: note = Azaglycine
  SITE 9: note = 4-amino-1-carbamimidoylPiperidine-2-carboxylic acideridine-4-carboxylic acid
  SITE 10: note = Amidated Tryptophan
GYKNTFGLXW

SEQ ID NO: 725 - protein, 10 aa, synthetic construct
  SITE 1: note = (3-fluoro-4-methylbenzyl)glycine
  SITE 2: note = D-Tyrosine
  SITE 3: note = D-Lysine(1,4,7,10-tetraazacyclododecane-1,4,7,10-te traacetic acid)
  SITE 7: note = Azaglycine
  SITE 9: note = 4-amino-1-carbamimidoylPiperidine-2-carboxylic acideridine-4-carboxylic acid
  SITE 10: note = Amidated Tryptophan
GYKNTFGLXW

SEQ ID NO: 726 - protein, 9 aa, synthetic construct
  SITE 1: note = 1-(3-fluoro-4-methylphenyl)-4-oxo-8,11-dioxa-2,5-di azatetradecan-14-oic acid-D-Tyrosine
  SITE 2: note = D-Lysine(1,4,7,10-tetraazacyclododecane-1,4,7,10-te traacetic acid)
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YKNTFGLXW

SEQ ID NO: 727 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = 4-benzoylphenylalanine
  SITE 7: note = Azaglycine
  SITE 9: note = 4-amino-1-carbamimidoylpiperidine-4-carboxylic acid(Sarcosine)
  SITE 10: note = Amidated D-Tryptophan
XYXNTFGLXW

SEQ ID NO: 728 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = 4-benzoylphenylalanine
  SITE 7: note = Azaglycine
  SITE 9: note = 4-amino-1-carbamimidoylpiperidine-4-carboxylic acid(Sarcosine)
  SITE 10: note = Amidated Tryptophan
XYXNTFGLXW

SEQ ID NO: 729 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = biphenylalanine
  SITE 7: note = Azaglycine
  SITE 9: note = 4-amino-1-carbamimidoylpiperidine-4-carboxylic acid(Sarcosine)
  SITE 10: note = Amidated Tryptophan
XYXNTFGLXW

SEQ ID NO: 730 - protein, 9 aa, synthetic construct
  SITE 1: note = dodecanoic acid-D-Tyrosine
  SITE 2: note = D-Lysine(1,4,7,10-tetraazacyclododecane-1,4,7,10-te traacetic acid)
  SITE 6: note = Azaglycine
  SITE 8: note = 4-amino-1-carbamimidoylPiperidine-2-carboxylic acideridine-4-carboxylic acid
  SITE 9: note = Amidated Tyrosine
YKNTFGLXY

SEQ ID NO: 731 - protein, 10 aa, synthetic construct
  SITE 1: note = (3-(4-(l5-methyl)phenyl)propanoyl)-D-glutamic acid
  SITE 2: note = D-Tyrosine
  SITE 3: note = Lysine(1,4,7,10-tetraazacyclododecane-1,4,7,10-tetr aacetic acid)
  SITE 7: note = Azaglycine
  SITE 9: note = 4-amino-1-carbamimidoylPiperidine-2-carboxylic acideridine-4-carboxylic acid
  SITE 10: note = Amidated Tryptophan
EYKNTFGLXW

SEQ ID NO: 732 - protein, 9 aa, synthetic construct
  SITE 1: note = octanoic acid-D-Tyrosine
  SITE 2: note = D-Lysine(1,4,7,10-tetraazacyclododecane-1,4,7,10-te traacetic acid)
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YKNTFGLXW

SEQ ID NO: 733 - protein, 9 aa, synthetic construct
  SITE 1: note = octanoic acid-D-Tyrosine
  SITE 2: note = D-Lysine(1,4,7,10-tetraazacyclododecane-1,4,7,10-te traacetic acid)
  SITE 5: note = cyclohexylalanine
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YKNTXGLXW

SEQ ID NO: 734 - protein, 9 aa, synthetic construct
  SITE 1: note = 3-(2-(2-(3-(p-tolyl)propanamido)ethoxy)ethoxy)propa noic acid-D-Tyrosine
  SITE 2: note = D-Lysine(1,4,7,10-tetraazacyclododecane-1,4,7,10-te traacetic acid)
  SITE 5: note = cyclohexylalanine
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YKNTXGLXW

SEQ ID NO: 735 - protein, 9 aa, synthetic construct
  SITE 1: note = octanoic acid-D-Tyrosine
  SITE 2: note = D-Lysine(1,4,7,10-tetraazacyclododecane-1,4,7,10-te traacetic acid)
  SITE 6: note = Azaglycine
  SITE 8: note = 4-amino-1-carbamimidoylpiperidine-4-carboxylic acid(Sarcosine)
  SITE 9: note = Amidated D-Tryptophan
YKNTFGLXW

SEQ ID NO: 736 - protein, 9 aa, synthetic construct
  SITE 1: note = octanoic acid-D-Tyrosine
  SITE 2: note = D-Lysine(1,4,7,10-tetraazacyclododecane-1,4,7,10-te traacetic acid)
  SITE 6: note = Azaglycine
  SITE 8: note = 4-amino-1-carbamimidoylpiperidine-4-carboxylic acid(Sarcosine)
  SITE 9: note = Amidated Tryptophan
YKNTFGLXW

SEQ ID NO: 737 - protein, 9 aa, synthetic construct
  SITE 1: note = octanoic acid-D-Tyrosine
  SITE 2: note = D-Lysine(1,4,7,10-tetraazacyclododecane-1,4,7,10-te traacetic acid)
  SITE 5: note = cyclohexylalanine
  SITE 6: note = Azaglycine
  SITE 8: note = 4-amino-1-carbamimidoylpiperidine-4-carboxylic acid(Sarcosine)
  SITE 9: note = Amidated D-Tryptophan
YKNTXGLXW

SEQ ID NO: 738 - protein, 9 aa, synthetic construct
  SITE 1: note = 3-(2-(2-(3-(p-tolyl)propanamido)ethoxy)ethoxy)propa noic acid-D-Tyrosine
  SITE 2: note = Lysine(1,4,7,10-tetraazacyclododecane-1,4,7,10-tetr aacetic acid)
  SITE 5: note = cyclohexylalanine
  SITE 6: note = Azaglycine
  SITE 8: note = 4-amino-1-carbamimidoylpiperidine-4-carboxylic acid(Sarcosine)
  SITE 9: note = Amidated D-Tryptophan
YKNTXGLXW

SEQ ID NO: 739 - protein, 9 aa, synthetic construct
  SITE 1: note = 3-(2-(2-(3-(p-tolyl)propanamido)ethoxy)ethoxy)propa noic acid-D-Tyrosine
  SITE 2: note = Lysine(1,4,7,10-tetraazacyclododecane-1,4,7,10-tetr aacetic acid)
  SITE 5: note = cyclohexylalanine
  SITE 6: note = Azaglycine
  SITE 8: note = 4-amino-1-carbamimidoylpiperidine-4-carboxylic acid(Sarcosine)
  SITE 9: note = Amidated Tryptophan
YKNTXGLXW

SEQ ID NO: 740 - protein, 9 aa, synthetic construct
  SITE 1: note = 3-(2-(2-(3-(p-tolyl)propanamido)ethoxy)ethoxy)propa noic acid-D-Tyrosine
  SITE 2: note = D-Lysine(1,4,7,10-tetraazacyclododecane-1,4,7,10-te traacetic acid)
  SITE 5: note = cyclohexylalanine
  SITE 6: note = Azaglycine
  SITE 8: note = 4-amino-1-carbamimidoylpiperidine-4-carboxylic acid(Sarcosine)
  SITE 9: note = Amidated D-Tryptophan
YKNTXGLXW

SEQ ID NO: 741 - protein, 9 aa, synthetic construct
  SITE 1: note = 1-(3-fluoro-4-methylphenyl)-4-oxo-8,11-dioxa-2,5-di azatetradecan-14-oic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylpiperidine-4-carboxylic acid(Sarcosine)
  SITE 9: note = Amidated D-Tryptophan
YKNTFGLXW

SEQ ID NO: 742 - protein, 10 aa, synthetic construct
  SITE 1: note = (3-fluoro-4-methylbenzyl)glycine
  SITE 2: note = D-Tyrosine
  SITE 3: note = Lysine(1,4,7,10-tetraazacyclododecane-1,4,7,10-tetr aacetic acid)
  SITE 7: note = Azaglycine
  SITE 9: note = 4-amino-1-carbamimidoylpiperidine-4-carboxylic acid(Sarcosine)
  SITE 10: note = Amidated D-Tryptophan
GYKNTFGLXW

SEQ ID NO: 743 - protein, 9 aa, synthetic construct
  SITE 1: note = 1-(3-fluoro-4-methylphenyl)-4-oxo-8,11-dioxa-2,5-di azatetradecan-14-oic acid-D-Tyrosine
  SITE 2: note = D-Lysine(1,4,7,10-tetraazacyclododecane-1,4,7,10-te traacetic acid)
  SITE 6: note = Azaglycine
  SITE 8: note = 4-amino-1-carbamimidoylpiperidine-4-carboxylic acid(Sarcosine)
  SITE 9: note = Amidated Tryptophan
YKNTFGLXW

SEQ ID NO: 744 - protein, 9 aa, synthetic construct
  SITE 1: note = (3-fluoro-4-methylbenzyl)glycine
  SITE 2: note = D-Tyrosine
  SITE 2: note = D-Lysine(1,4,7,10-tetraazacyclododecane-1,4,7,10-te traacetic acid)
  SITE 6: note = Azaglycine
  SITE 8: note = 4-amino-1-carbamimidoylpiperidine-4-carboxylic acid(Sarcosine)
  SITE 9: note = Amidated Tryptophan
YKNTFGLXW

SEQ ID NO: 745 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = biphenylalanine
  SITE 7: note = Azaglycine
  SITE 9: note = 4-amino-1-((3-fluoro-4-methylbenzyl)glycyl)Piperidi ne-2-carboxylic acideridine-4-carboxamide
  SITE 10: note = Amidated Tryptophan
XYXNTFGLXW

SEQ ID NO: 746 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = 4-benzoylphenylalanine
  SITE 7: note = Azaglycine
  SITE 9: note = 4-amino-1-((3-fluoro-4-methylbenzyl)glycyl)Piperidi ne-2-carboxylic acideridine-4-carboxamide
  SITE 10: note = Amidated Tryptophan
XYXNTFGLXW

SEQ ID NO: 747 - protein, 10 aa, synthetic construct
  SITE 1: note = 2,2',2'',2'''-(1,4,7,10-tetraazacyclododecane-1,4,7 ,10-tetrayl)tetraacetic acid-Gly
  SITE 2: note = D-Tyrosine
  SITE 3: note = D-3-(2-Naphthyl)alanine
  SITE 7: note = Azaglycine
  SITE 9: note = 4-amino-1-((3-fluoro-4-methylbenzyl)glycyl)Piperidi ne-2-carboxylic acideridine-4-carboxamide
  SITE 10: note = Amidated Tryptophan
GYXNTFGLXW

SEQ ID NO: 748 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((3-fluoro-4-methylbenzyl)glycyl)Piperidi ne-2-carboxylic acideridine-4-carboxamide
  SITE 10: note = Amidated Tyrosine
XYWNTFGLXY

SEQ ID NO: 749 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = 4-amino-1-((3-fluoro-4-methylbenzyl)glycyl)Piperidi ne-2-carboxylic acideridine-4-carboxamide
  SITE 10: note = Amidated Tyrosine
XYXNTFGLXY

SEQ ID NO: 750 - protein, 9 aa, synthetic construct
  SITE 1: note = 2,2',2''-(10-(2-((2-(2-(2-carboxyethoxy)ethoxy)ethy l)amino)-2-oxoethyl)-1,4,7,10-tetraazacyclododecane-1,4,7- triyl)triacetic acid-D-Tyrosine
  SITE 2: note = D-3-(4-pyridyl)alanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
YXNTFGLXW

SEQ ID NO: 751 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = 4-amino-1-(((1-methylPiperidine-2-carboxylic acideridin-4-yl)methyl)glycyl)Piperidine-2-carboxylic acideridine-4-carboxylic acid
  SITE 10: note = Amidated Tryptophan
XYXNTFGLXW

SEQ ID NO: 752 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = 4-amino-1-((pyridin-4-ylmethyl)glycyl)Piperidine-2- carboxylic acideridine-4-carboxylic acid
  SITE 10: note = Amidated D-Tryptophan
XYXNTFGLXW

SEQ ID NO: 753 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = 4-amino-1-((4-iodobenzyl)glycyl)Piperidine-2-carbox ylic acideridine-4-carboxylic acid
  SITE 10: note = Amidated D-Tryptophan
XYXNTFGLXW

SEQ ID NO: 754 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = 1-(((1H-pyrrol-2-yl)methyl)glycyl)-4-aminoPiperidin e-2-carboxylic acideridine-4-carboxylic acid
  SITE 10: note = Amidated Tryptophan
XYXNTFGLXW

SEQ ID NO: 755 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = 4-amino-1-((4-iodophenethyl)glycyl)Piperidine-2-car boxylic acideridine-4-carboxylic acid
  SITE 10: note = Amidated D-Tryptophan
XYXNTFGLXW

SEQ ID NO: 756 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = 4-amino-1-(octylglycyl)Piperidine-2-carboxylic acideridine-4-carboxylic acid
  SITE 10: note = Amidated D-Tryptophan
XYXNTFGLXW

SEQ ID NO: 757 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = 4-amino-1-(((tetrahydro-2H-thiopyran-4-yl)methyl)gl ycyl)Piperidine-2-carboxylic acideridine-4-carboxylic acid
  SITE 10: note = Amidated D-Tryptophan
XYXNTFGLXW

SEQ ID NO: 758 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = 4-amino-1-((naphthalen-2-ylmethyl)glycyl)Piperidine -2-carboxylic acideridine-4-carboxylic acid
  SITE 10: note = Amidated D-Tryptophan
XYXNTFGLXW

SEQ ID NO: 759 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = 3-(2-Naphthyl)alanine or
  SITE 7: note = Azaglycine
  SITE 9: note = 4-amino-1-((3-fluoro-4-methylbenzyl)glycyl)Piperidi ne-2-carboxylic acideridine-4-carboxamide
  SITE 10: note = Amidated Tyrosine
XYXNTFGLXY

SEQ ID NO: 760 - protein, 9 aa, synthetic construct
  SITE 1: note = 3-(2-(2-hydroxyethoxy)ethoxy)propanoic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
YKNTFGLXW

SEQ ID NO: 761 - protein, 9 aa, synthetic construct
  SITE 1: note = Acetyl-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
YKNTFGLXW

SEQ ID NO: 762 - protein, 9 aa, synthetic construct
  SITE 1: note = octanoic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
YKNTFGLXW

SEQ ID NO: 763 - protein, 9 aa, synthetic construct
  SITE 1: note = 2,2',2''-(10-(2-((2-(2-(2-carboxyethoxy)ethoxy)ethy l)amino)-2-oxoethyl)-1,4,7,10-tetraazacyclododecane-1,4,7- triyl)triacetic acid-D-Tyrosine
  SITE 2: note = D-Tryptophan
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
YWNTFGLXW

SEQ ID NO: 764 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-Tryptophan
  SITE 7: note = Azaglycine
  SITE 9: note = 4-amino-1-((3-fluoro-4-methylbenzyl)glycyl)Piperidi ne-2-carboxylic acideridine-4-carboxamide
  SITE 10: note = Amidated D-Tryptophan
XYWNTFGLXW

SEQ ID NO: 765 - protein, 9 aa, synthetic construct
  SITE 1: note = 1-(3-fluoro-4-methylphenyl)-4-oxo-8,11-dioxa-2,5-di azatetradecan-14-oic acid-D-Tyrosine
  SITE 2: note = Lysine(1,4,7,10-tetraazacyclododecane-1,4,7,10-tetr aacetic acid)
  SITE 6: note = Azaglycine
  SITE 8: note = 4-amino-1-carbamimidoylpiperidine-4-carboxylic acid(Sarcosine)
  SITE 9: note = Amidated Tryptophan
YKNTFGLXW

SEQ ID NO: 766 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = D-3-(2-Naphthyl)alanine
  SITE 7: note = Azaglycine
  SITE 9: note = 4-amino-1-(octylglycyl)Piperidine-2-carboxylic acideridine-4-carboxylic acid
  SITE 10: note = Amidated Tryptophan
XYXNTFGLXW

SEQ ID NO: 767 - protein, 9 aa, synthetic construct
  SITE 1: note = (3-fluoro-4-methylbenzyl)glycine
  SITE 2: note = D-Tyrosine
  SITE 3: note = Lysine(1,4,7,10-tetraazacyclododecane-1,4,7,10-tetr aacetic acid)
  SITE 6: note = Azaglycine
  SITE 8: note = 4-amino-1-carbamimidoylpiperidine-4-carboxylic acid(Sarcosine)
  SITE 9: note = Amidated Tryptophan
YKNTFGLXW

SEQ ID NO: 768 - protein, 9 aa, synthetic construct
  SITE 1: note = 3-(2-(2-(3-(p-tolyl)propanamido)ethoxy)ethoxy)propa noic acid-D-Tyrosine
  SITE 2: note = Lysine(1,4,7,10-tetraazacyclododecane-1,4,7,10-tetr aacetic acid)
  SITE 5: note = cyclohexylalanine
  SITE 6: note = Azaglycine
  SITE 8: note = 4-amino-1-carbamimidoylpiperidine-4-carboxylic acid(Sarcosine)
  SITE 9: note = Amidated Tryptophan
YKNTXGLXW

SEQ ID NO: 769 - protein, 9 aa, synthetic construct
  SITE 1: note = 3-(2-(2-(3-(p-tolyl)propanamido)ethoxy)ethoxy)propa noic acid-D-Tyrosine
  SITE 2: note = D-Lysine(1,4,7,10-tetraazacyclododecane-1,4,7,10-te traacetic acid)
  SITE 5: note = cyclohexylalanine
  SITE 6: note = Azaglycine
  SITE 8: note = 4-amino-1-carbamimidoylpiperidine-4-carboxylic acid(Sarcosine)
  SITE 9: note = Amidated Tryptophan
YKNTXGLXW

SEQ ID NO: 770 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tyrosine
  SITE 3: note = biphenylalanine
  SITE 7: note = Azaglycine
  SITE 9: note = 4-amino-1-carbamimidoylpiperidine-4-carboxylic acid(Sarcosine)
  SITE 10: note = Amidated D-Tryptophan
XYXNTFGLXW

SEQ ID NO: 771 - protein, 6 aa, synthetic construct
  SITE 1: note = indium(III) 2,2',2''-(10-(2-((5-carboxypentyl)amino )-2-oxoethyl)-1,4,7,10-tetraazacyclododecane-1,4,7-triyl)t riacetate
  SITE 3: note = Azaglycine
  SITE 5: note = 4-amino-1-((3-fluoro-4-methylbenzyl)glycyl)Piperidi ne-2-carboxylic acideridine-4-carboxamide
  SITE 6: note = Amidated Tryptophan
XFGLXW

SEQ ID NO: 772 - protein, 10 aa, synthetic construct
  SITE 1: note = indium(III) 2,2',2''-(10-(2-((5-carboxypentyl)amino )-2-oxoethyl)-1,4,7,10-tetraazacyclododecane-1,4,7-triyl)t riacetate
  SITE 2: note = D-Tyrosine
  SITE 3: note = D-Tryptophan
  SITE 7: note = Azaglycine
  SITE 9: note = 4-amino-1-carbamimidoylPiperidine-2-carboxylic acideridine-4-carboxylic acid
  SITE 10: note = Amidated Tryptophan
XYWNTFGLXW

SEQ ID NO: 773 - skipped

SEQ ID NO: 774 - protein, 5 aa, synthetic construct
  SITE 1: note = 3-Fluorophenylalanine
  SITE 2: note = Azaglycine
  SITE 4: note = 4-amino-1-R1-Piperidine-2-carboxylic acideridine-4-carboxylic acid
  SITE 5: note = Amidated 4-cyano phenylalanine
FGLXF

SEQ ID NO: 775 - protein, 5 aa, synthetic construct
  SITE 1: note = 3-Fluorophenylalanine
  SITE 2: note = Azaglycine
  SITE 4: note = 4-amino-1-R1-Piperidine-2-carboxylic acideridine-4-carboxylic acid
  SITE 5: note = Amidated Tryptophan
FGLXW

SEQ ID NO: 776-785 - skipped (10 entries)

SEQ ID NO: 786 - protein, 5 aa, synthetic construct
  SITE 2: note = Azaglycine
  SITE 4: note = 4-amino-1-R1-Piperidine-2-carboxylic acideridine-4-carboxylic acid
  SITE 5: note = Amidated Leucine
FGLXL

SEQ ID NO: 787-789 - skipped (3 entries)

SEQ ID NO: 790 - protein, 5 aa, synthetic construct
  SITE 1: note = AzaPhe
  SITE 2: note = Azaglycine
  SITE 4: note = 4-amino-1-((3-fluoro-4-methylbenzyl)glycyl)Piperidi ne-2-carboxylic acideridine-4-carboxamide
  SITE 5: note = Amidated Tyrosine
FGLXY

SEQ ID NO: 791 - skipped

SEQ ID NO: 792 - protein, 5 aa, synthetic construct
  SITE 1: note = beta-(2-thienyl)-Ala
  SITE 2: note = Azaglycine
  SITE 4: note = 4-amino-1-R1-Piperidine-2-carboxylic acideridine-4-carboxylic acid
  SITE 5: note = Amidated Tryptophan
AGLXW

SEQ ID NO: 793 - protein, 5 aa, synthetic construct
  SITE 2: note = Azaglycine
  SITE 4: note = 4-amino-1-R1-Piperidine-2-carboxylic acideridine-4-carboxylic acid
  SITE 5: note = Trp(3-NO2)-OH-NH2
AGLXW

SEQ ID NO: 794-795 - skipped (2 entries)

SEQ ID NO: 796 - protein, 7 aa, synthetic construct
  SITE 1: note = 4-(aminomethyl)benzoic acid
  SITE 4: note = 4-(aminomethyl)benzoic acid
  SITE 7: note = N6-(5-carboxypentanoyl)-L-lysine
XMVXMVX

SEQ ID NO: 797 - protein, 6 aa, synthetic construct
  SITE 1: note = 6-(DOTA-amino)hexanoic acid
  SITE 2: note = Sarcosine
  SITE 3: note = Sarcosine
  SITE 4: note = Sarcosine
  SITE 5: note = Sarcosine
  SITE 6: note = Sarcosine
XGGGGG

SEQ ID NO: 798 - protein, 6 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = Sarcosine
  SITE 3: note = Sarcosine
  SITE 4: note = Sarcosine
  SITE 5: note = Sarcosine
XGGGGG

SEQ ID NO: 799 - protein, 7 aa, synthetic construct
  SITE 1: note = 2,2',2'',2'''-(1,4,7,10-tetraazacyclododecane-1,4,7 ,10-tetrayl)tetraacetic acid coupled to N-term of 4-(aminomethyl)benzoic acid (4-(aminomethyl)benzoic acid)
  SITE 4: note = 4-(aminomethyl)benzoic acid
  SITE 7: note = N6-(5-carboxypentanoyl)-L-lysine
XMVXMVX

SEQ ID NO: 800 - protein, 8 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 5: note = Azaglycine
  SITE 7: note = 1-amino-4-guanidinocyclohexane-1-carboxylic acid
  SITE 8: note = Amidated Tryptophan
XNTFGLXW

SEQ ID NO: 801 - protein, 10 aa, synthetic construct
  SITE 1: note = 2,2',2'',2'''-(1,4,7,10-tetraazacyclododecane-1,4,7 ,10-tetrayl)tetraacetic acid 6-aminohexanoic acid
  SITE 2: note = D-Tryptophan
  SITE 6: note = Azaglycine
  SITE 8: note = 1-amino-4-guanidinocyclohexane-1-carboxylic acid
  SITE 9: note = Amidated Tryptophan
XYWNTFGLXW

SEQ ID NO: 802 - protein, 9 aa, synthetic construct
  SITE 1: note = Acetyl-D-Tyrosine
  SITE 2: note = D-Tryptophan
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
YWNTFGLXW

SEQ ID NO: 803 - protein, 9 aa, synthetic construct
  SITE 1: note = Acetyl-D-Tyrosine
  SITE 2: note = D-Tryptophan
  SITE 5: note = cyclohexylalanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
YWNTXGLXW

SEQ ID NO: 804 - protein, 5 aa, synthetic construct
  SITE 1: note = Acetyl-Phe
  SITE 2: note = Azaglycine
  SITE 4: note = 4-amino-1-((3-fluoro-4-methylbenzyl)glycyl)Piperidi ne-2-carboxylic acideridine-4-carboxamide
  SITE 5: note = Amidated Tryptophan
FGLXW

SEQ ID NO: 805 - protein, 9 aa, synthetic construct
  SITE 1: note = Acetyl-D-Tyrosine
  SITE 2: note = D-3-(2-naphthyl)-alanine
  SITE 6: note = Azaglycine
  SITE 8: note = 4-amino-1-carbamimidoylpiperidine-4-carboxylic acid(Sarcosine)
  SITE 9: note = Amidated Tryptophan
YXNTFGLXW

SEQ ID NO: 806 - protein, 9 aa, synthetic construct
  SITE 1: note = 3-(2-(2-(3-(4-iodophenyl)propanamido)ethoxy)ethoxy) propanoic acid coupled to N-term of D-Tyrosine
  SITE 2: note = D-3-(2-naphthyl)-alanine
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YXNTFGLXW

SEQ ID NO: 807 - protein, 9 aa, synthetic construct
  SITE 1: note = 4-(4-iodophenyl)butanoic acid coupled to N-term of D-Tyrosine
  SITE 2: note = D-3-(2-naphthyl)-alanine
  SITE 6: note = Azaglycine
  SITE 8: note = 4-amino-1-carbamimidoylpiperidine-4-carboxylic acid(Sarcosine)
  SITE 9: note = Amidated Tryptophan
YXNTFGLXW

SEQ ID NO: 808 - protein, 9 aa, synthetic construct
  SITE 1: note = 3-(2-(2-(3-(4-iodophenyl)propanamido)ethoxy)ethoxy) propanoic acid coupled to N-term of D-Tyrosine
  SITE 2: note = D-3-(2-naphthyl)-alanine
  SITE 6: note = Azaglycine
  SITE 8: note = 4-amino-1-carbamimidoylpiperidine-4-carboxylic acid(Sarcosine)
  SITE 9: note = Amidated Tryptophan
YXNTFGLXF

SEQ ID NO: 809 - protein, 9 aa, synthetic construct
  SITE 1: note = 3-(2-(2-(3-(4-iodophenyl)propanamido)ethoxy)ethoxy) propanoic acid coupled to N-term of D-Tyrosine
  SITE 2: note = D-3-(2-naphthyl)-alanine
  SITE 6: note = Azaglycine
  SITE 8: note = 4-amino-1-carbamimidoylPiperidine-2-carboxylic acideridine-4-carboxylic acid
  SITE 9: note = Amidated Phenylalanine
YXNTFGLXY

SEQ ID NO: 810 - protein, 9 aa, synthetic construct
  SITE 1: note = 4-(4-iodophenyl)butanoic acid coupled to N-term of D-Tyrosine
  SITE 2: note = D-3-(2-naphthyl)-alanine
  SITE 6: note = Azaglycine
  SITE 8: note = 4-amino-1-carbamimidoylpiperidine-4-carboxylic acid(Sarcosine)
  SITE 9: note = Amidated Phenylalanine
YXNTFGLXF

SEQ ID NO: 811 - protein, 9 aa, synthetic construct
  SITE 1: note = 4-(4-iodophenyl)butanoic acid coupled to N-term of D-Tyrosine
  SITE 2: note = D-3-(2-naphthyl)-alanine
  SITE 6: note = Azaglycine
  SITE 8: note = 4-amino-1-carbamimidoylpiperidine-4-carboxylic acid(Sarcosine)
  SITE 9: note = Amidated Tyrosine
YXNTFGLXY

SEQ ID NO: 812 - protein, 9 aa, synthetic construct
  SITE 1: note = dodecanoic acid coupled to N-term of D-Tyrosine
  SITE 2: note = D-3-(2-naphthyl)-alanine
  SITE 6: note = Azaglycine
  SITE 8: note = 4-amino-1-carbamimidoylpiperidine-4-carboxylic acid(Sarcosine)
  SITE 9: note = Amidated Tyrosine
YXNTFGLXW

SEQ ID NO: 813 - protein, 9 aa, synthetic construct
  SITE 1: note = dodecanoic acid coupled to N-term of D-Tyrosine
  SITE 2: note = D-3-(2-naphthyl)-alanine
  SITE 6: note = Azaglycine
  SITE 8: note = 4-amino-1-carbamimidoylpiperidine-4-carboxylic acid(Sarcosine)
  SITE 9: note = Amidated Tyrosine
YXNTFGLXY

SEQ ID NO: 814 - protein, 9 aa, synthetic construct
  SITE 1: note = D-Tyrosine
  SITE 2: note = D-Tryptophan
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
YWNTFGLXW

SEQ ID NO: 815 - protein, 9 aa, synthetic construct
  SITE 1: note = 4-(p-tolyl)butanoic acid coupled to N-term of D-Tyrosine
  SITE 2: note = D-Tryptophan
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
YWNTFGLXW

SEQ ID NO: 816 - protein, 9 aa, synthetic construct
  SITE 1: note = D-Tyrosine
  SITE 2: note = D-Tryptophan
  SITE 5: note = cyclohexylalanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
YWNTXGLXW

SEQ ID NO: 817 - protein, 9 aa, synthetic construct
  SITE 1: note = Octanoic acid coupled to N-term of D-Tyrosine
  SITE 2: note = D-Tryptophan
  SITE 5: note = cyclohexylalanine
  SITE 6: note = Azaglycine
  SITE 8: note = 4-amino-1-((3-fluoro-4-methylbenzyl)glycyl)Piperidi ne-2-carboxylic acideridine-4-carboxamide
  SITE 9: note = Amidated Tryptophan
YWNTXGLXW

SEQ ID NO: 818 - protein, 9 aa, synthetic construct
  SITE 1: note = D-Tyrosine
  SITE 2: note = D-3-(2-naphthyl)-alanine
  SITE 6: note = Azaglycine
  SITE 8: note = 4-amino-1-carbamimidoylpiperidine-4-carboxylic acid(Sarcosine)
  SITE 9: note = Amidated Phenylalanine
YXNTFGLXF

SEQ ID NO: 819 - protein, 9 aa, synthetic construct
  SITE 1: note = D-Tyrosine
  SITE 2: note = D-3-(2-naphthyl)-alanine
  SITE 6: note = Azaglycine
  SITE 8: note = 4-amino-1-carbamimidoylpiperidine-4-carboxylic acid(Sarcosine)
  SITE 9: note = Amidated Tyrosine
YXNTFGLXY

SEQ ID NO: 820 - protein, 9 aa, synthetic construct
  SITE 1: note = D-Tyrosine
  SITE 2: note = D-3-(2-naphthyl)-alanine
  SITE 6: note = Azaglycine
  SITE 8: note = 4-amino-1-carbamimidoylpiperidine-4-carboxylic acid(Sarcosine)
  SITE 9: note = Amidated Tryptophan
YXNTFGLXW

SEQ ID NO: 821 - protein, 9 aa, synthetic construct
  SITE 1: note = D-Tyrosine
  SITE 2: note = D-3-(2-naphthyl)-alanine
  SITE 6: note = Azaglycine
  SITE 8: note = 4-amino-1-carbamimidoylPiperidine-2-carboxylic acideridine-4-carboxylic acid
  SITE 9: note = Amidated Phenylalanine
YXNTFGLXF

SEQ ID NO: 822 - protein, 9 aa, synthetic construct
  SITE 1: note = 3-(2-(2-aminoethoxy)ethoxy)propanoic acid coupled to N-term of D-Tyrosine
  SITE 2: note = D-3-(2-naphthyl)-alanine
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YXNTFGLXW

SEQ ID NO: 823 - protein, 9 aa, synthetic construct
  SITE 1: note = D-Tyrosine
  SITE 2: note = D-3-(2-naphthyl)-alanine
  SITE 6: note = Azaglycine
  SITE 8: note = 4-amino-1-carbamimidoylPiperidine-2-carboxylic acideridine-4-carboxylic acid
  SITE 9: note = Amidated Tryptophan
YXNTFGLXW

SEQ ID NO: 824 - protein, 9 aa, synthetic construct
  SITE 1: note = (R)-3-(4-(tert-butoxy)phenyl)-2-(6-(2-(4,7,10-tris( 2-(tert-butoxy)-2-oxoethyl)-1,4,7,10-tetraazacyclododecan- 1-yl)acetamido)hexanamido)propanoic acid
  SITE 2: note = 1-(tert-butoxycarbonyl)-D-tryptophan
  SITE 3: note = N4-trityl-L-asparagine
  SITE 4: note = O-(tert-butyl)-L-threonine
  SITE 6: note = Azaglycine
  SITE 8: note = 4-amino-1-(N-(tert-butoxycarbonyl)-N-(3-fluoro-4-me thylbenzyl)glycyl)Piperidine-2-carboxylic acideridine-4-carboxylic acid
  SITE 9: note = 1-(tert-butoxycarbonyl)-L-tryptophan
XWDTFGLXW

SEQ ID NO: 825 - protein, 9 aa, synthetic construct
  SITE 1: note = (R)-3-(4-(tert-butoxy)phenyl)-2-octanamidopropanoic acid
  SITE 2: note = N6-(1-(4,4-dimethyl-2,6-dioxocyclohexylidene)ethyl) -L-lysine
  SITE 3: note = N4-trityl-L-asparagine
  SITE 4: note = O-(tert-butyl)-L-threonine
  SITE 6: note = Azaglycine
  SITE 3: note = N4-trityl-L-asparagine
  SITE 4: note = O-(tert-butyl)-L-threonine
  SITE 8: note = 4-amino-1-(tert-butoxycarbonyl)Piperidine-2-carboxy lic acideridine-4-carboxylic acid
  SITE 9: note = (S)-2-amino-3-(4-(tert-butoxy)phenyl)propanoic acid
XKDTFGLXX

SEQ ID NO: 826 - protein, 9 aa, synthetic construct
  SITE 1: note = (R)-3-(4-(tert-butoxy)phenyl)-2-octanamidopropanoic acid
  SITE 2: note = (S)-2,2',2''-(10-(2-((5-amino-5-carboxypentyl)amino )-2-oxoethyl)-1,4,7,10-tetraazacyclododecane-1,4,7-triyl)t riacetic acid
  SITE 3: note = N4-trityl-L-asparagine
  SITE 4: note = O-(tert-butyl)-L-threonine
  SITE 6: note = Azaglycine
  SITE 8: note = 4-amino-1-(tert-butoxycarbonyl)Piperidine-2-carboxy lic acideridine-4-carboxylic acid
  SITE 9: note = (S)-2-amino-3-(4-(tert-butoxy)phenyl)propanoic acid
XXDTFGLXX

SEQ ID NO: 827 - protein, 9 aa, synthetic construct
  SITE 1: note = D-Tyrosine
  SITE 2: note = (S)-2,2',2''-(10-(2-((5-amino-5-carboxypentyl)amino )-2-oxoethyl)-1,4,7,10-tetraazacyclododecane-1,4,7-triyl)t riacetic acid
  SITE 6: note = Azaglycine
  SITE 8: note = 4-aminoPiperidine-2-carboxylic acideridine-4-carboxylic acid
  SITE 9: note = Amidated Tyrosine
YXNTFGLXY

SEQ ID NO: 828 - protein, 10 aa, synthetic construct
YNWNSFGLRF

SEQ ID NO: 829 - protein, 4 aa, synthetic construct
  SITE 2: note = D-Lysine
YKNT

SEQ ID NO: 830 - protein, 4 aa, synthetic construct
  SITE 1: note = D-Tyrosine
  SITE 2: note = D-Lysine
YKNT

SEQ ID NO: 831 - protein, 6 aa, synthetic construct
  SITE 1: note = Sarcosine
  SITE 2: note = Sarcosine
  SITE 3: note = Sarcosine
  SITE 4: note = Sarcosine
  SITE 5: note = Sarcosine
  SITE 6: note = Sarcosine
GGGGGG

SEQ ID NO: 832 - protein, 4 aa, synthetic construct
GFGG

SEQ ID NO: 833 - protein, 6 aa, synthetic construct
  VARIANT 2..6: note = Residues may be deleted
GGGGGG

SEQ ID NO: 834 - protein, 6 aa, synthetic construct
  SITE 1..6: note = beta-Alanine
  VARIANT 2..6: note = Residues may be deleted
AAAAAA

SEQ ID NO: 835 - protein, 6 aa, synthetic construct
  VARIANT 2..6: note = Residues may be deleted
AAAAAA

SEQ ID NO: 836 - protein, 6 aa, synthetic construct
  VARIANT 2..6: note = Residues may be deleted
EEEEEE

SEQ ID NO: 837 - protein, 6 aa, synthetic construct
  SITE 1..6: note = Sarcosine
  VARIANT 2..6: note = Residues may be deleted
GGGGGG

SEQ ID NO: 838 - protein, 6 aa, synthetic construct
  VARIANT 2..6: note = Residues may be deleted
NNNNNN

SEQ ID NO: 839 - protein, 7 aa, synthetic construct
  SITE 1: note = beta-Alanine
  VARIANT 2..7: note = Residues may be deleted
AGGGGGG

SEQ ID NO: 840 - protein, 7 aa, synthetic construct
  SITE 1: note = beta-Alanine
  SITE 2..7: note = Sarcosine
  VARIANT 2..7: note = Residues may be deleted
AGGGGGG

SEQ ID NO: 841 - protein, 7 aa, synthetic construct
  SITE 1: note = beta-Alanine
  VARIANT 2..7: note = Residues may be deleted
AEEEEEE

SEQ ID NO: 842 - protein, 7 aa, synthetic construct
  SITE 1: note = beta-Alanine
  VARIANT 2..7: note = Residues may be deleted
ANNNNNN

SEQ ID NO: 843 - protein, 7 aa, synthetic construct
  VARIANT 2..6: note = Residues may be deleted
  SITE 7: note = beta-Alanine
EEEEEEX

SEQ ID NO: 844 - protein, 7 aa, synthetic construct
  SITE 1: note = 2,2',2'',2'''-(1,4,7,10-tetraazacyclododecane-1,4,7 ,10-tetrayl)tetraacetic acid-4-(aminomethyl)benzoic acid
  SITE 4: note = 4-(aminomethyl)benzoic acid
  SITE 7: note = N6-(5-carboxypentanoyl)-L-lysine
XMVXMVX

SEQ ID NO: 845 - protein, 4 aa, synthetic construct
  SITE 1: note = 2-(2-(2-hydroxyethoxy)ethoxy)acetic acid coupled to N-terminus of D-Tyrosine
  SITE 2: note = Lysine(2,2',2'',2'''-(1,4,7,10-tetraazacyclododecan e-1,4,7,10-tetrayl)tetraacetic acid)
YKNT

SEQ ID NO: 846 - protein, 7 aa, synthetic construct
  SITE 1: note = palmitic acid coupled to N-terminus of Glutamic Acid
  SITE 2: note = Lysine(2,2',2'',2'''-(1,4,7,10-tetraazacyclododecan e-1,4,7,10-tetrayl)tetraacetic acid)
EKYNWNS

SEQ ID NO: 847 - protein, 7 aa, synthetic construct
  SITE 1: note = Acetyl coupled to N-terminus of Glutamic Acid
  SITE 2: note = Lysine(2,2',2'',2'''-(1,4,7,10-tetraazacyclododecan e-1,4,7,10-tetrayl)tetraacetic acid)
  SITE 3: note = D-Alanine
EKANWNG

(19) United States
(12) Patent Application Publication
(10) Pub. No.: US 2025/0312499 A1
(43) Pub. Date: Oct. 9, 2025

(54) KISSPEPTIN RECEPTOR (KISS1R) TARGETED THERAPEUTICS AND USES THEREOF

(71) Applicant: Radionetics Oncology, Inc., San Diego, CA (US)

(72) Inventors: Junjie LIU, San Diego, CA (US); Yunfei ZHU, San Diego, CA (US); Yifeng XIONG, San Diego, CA (US); Jian ZHAO, San Diego, CA (US)

(21) Appl. No.: 19/172,351

(22) Filed: Apr. 7, 2025

Related U.S. Application Data
(60) Provisional application No. 63/631,177, filed on Apr. 8, 2024.
(60) Provisional application No. 63/683,591, filed on Aug. 15, 2024.

(51) Int. Cl. A61K 51/08

(52) U.S. Cl. CPC A61K 51/088; A61K 2121/00

(57) ABSTRACT

Described herein are radiotherapeutics that target tumor cells expressing the Kisspeptin receptor (KISS1R) and their use in the treatment and/or diagnosis of cancer.

### FIELD OF THE INVENTION

Described herein are radiotherapeutics that target tumor cells expressing Kisspeptin receptor (KISS1R) and methods of using such radiotherapeutics as cancer therapeutics, diagnostics, or both.

### SEQUENCE LISTING

The instant application contains a Sequence Listing which has been submitted electronically in XML format and is hereby incorporated by reference in its entirety. Said XML copy, created on Mar. 31, 2025, is named 63172-710_201 SL.xml and is 1,990,980 bytes in size.

### CROSS-REFERENCE TO RELATED APPLICATIONS

This application claims the benefit of U.S. Provisional Patent Application No. 63/631,177, filed Apr. 8, 2024, and U.S. Provisional Patent Application No. 63/683,591, filed Aug. 15, 2024, which are incorporated herein by reference in their entireties.

### BACKGROUND OF THE INVENTION

Neoplasms are abnormal growth of cells and cause enormous medical burdens, including morbidity and mortality, in humans. Neoplasms include benign or noncancerous neoplasms which do not display malignant features and are generally unlikely to become dangerous (e.g., adenomas). Malignant neoplasms display features such as genetic mutations, loss of normal function, rapid division, and ability metastasize (invade) to other tissues; and neoplasms of uncertain or unknown behavior. Malignant neoplasms (i.e., cancerous solid tumors) are the leading cause of death in industrialized countries. Noncancerous neoplasms including benign adenomas can also cause significant morbidity and mortality. Although standard treatments can achieve significant effects in tumor growth inhibition and even tumor elimination, the applied drugs exhibit only minor selectivity for the malignant tissue over healthy tissue and their severe side effects limit their efficacy and use. Specific targeting of neoplastic cells without affecting healthy tissue is a major desire for effective solid tumor therapy.

G protein-coupled receptors (GPCRs) are an important class of cell surface receptors that are frequently overexpressed in tumor cells and considered promising targets for selective tumor therapy. KISS1R, also referred as GPR54, is a GPCR overexpressed in several cancers, including, but not limited to, breast cancer, renal cell carcinoma, and lung cancer. Additionally, the kisspeptin/KISS1R signaling pathway is responsible for secretion of gonadotropin-releasing hormone (GnRH), an important modulator of the reproductive system. GnRH receptors are expressed in various tumor cells such as melanoma, prostate and endometrial carcinomas, leiomyomas, leiomyosarcomas, breast cancer, choriocarcinoma, epithelia and stromal tumors of the ovary. As such, targeted delivery of radionuclides to tumors with KISS1R-targeting conjugates offers a novel approach to treat and diagnose various cancers

### SUMMARY OF THE INVENTION

Described herein are radiopharmaceuticals for use in the diagnosis and/or treatment of tumors. The present disclosure provides an alternative and improved method for the treatment of tumors by targeting tumors that overexpress the Kisspeptin receptor (KISS1R). In some embodiments, the radiopharmaceuticals disclosed herein are useful in the treatment of tumors that overexpress KISS1R. In some other embodiments, the radiopharmaceuticals disclosed herein are useful in the identification of tissues or organs in a subject comprising tumors overexpressing KISS1R. The radiopharmaceuticals disclosed herein are also useful in vivo imaging of a subject for the presence of and distribution of tumors that overexpress KISS1R in the subject.

In one aspect, described herein is a compound of Formula (I), or a pharmaceutically acceptable salt thereof:

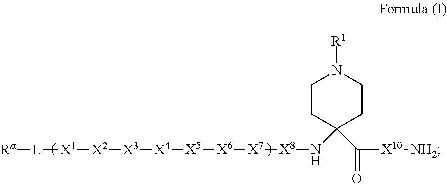

wherein:
R^{a} is a chelating moiety or a radionuclide complex thereof;
L is an optional linker that is attached to any one of X¹, X², X³, X⁴, X⁵, X⁶, or X⁷; or L is attached to X⁸ if X¹, X², X³, X⁴, X⁵, X⁶, and X⁷ are absent; and
R¹ is H,

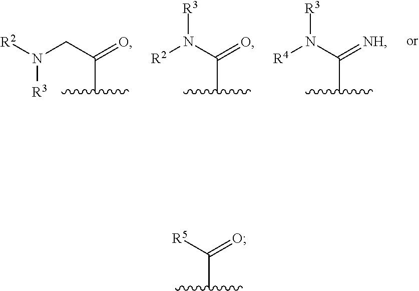

R² is C₁-C₆ alkyl, substituted or unsubstituted heteroalkyl, —((CHR⁶)ₙ-heterocycloalkyl, —(CHR⁶)ₙ-aryl, —(CHR⁶)ₙ-heteroaryl, —C(═O)—(CHR⁶)ₙ-aryl, or —C(═O)NH—(CHR⁶)ₙ-aryl; wherein C₁-C₆ alkyl is optionally substituted with R⁷, and wherein the heterocycloalkyl, aryl, or heteroaryl are each independently optionally substituted with R⁷, R⁸, R⁹, R¹⁰, and R¹¹;
R³ is H or C₁-C₄ alkyl;
R⁴ is H, C₁-C₄ alkyl, or R²,
R⁵ is substituted or unsubstituted alkyl, substituted or unsubstituted heteroalkyl, or —(CHR⁶)ₙ-aryl; wherein aryl is optionally substituted with R⁷, R⁸, R⁹, R¹⁰, and R¹¹
each R⁶ is independently H, F, —CH₃, —NH₂, or —OH;
R⁷, R⁸, R⁹, R¹⁰, and R¹¹ are each independently selected from H, F, Cl, Br, I, OH, —O—C₁-C₄ alkyl, —NH₂, —NHC₁-C₄ alkyl, —N(C₁-C₄ alkyl)₂, —CN, —CO₂H, —CO₂C₁-C₁ alkyl, —C₁-C₆ alkyl, —C₁-C₆ fluroroalkyl or —C₃-C₆ cycloalkyl;
n is 0, 1, 2, 3, 4, 5, or 6;
X¹ is absent, tyrosine (Tyr) glycine (Gly), sarcosine (Sar), alanine (Ala), aspartic acid (Asp), lysine (Lys), phenylalanine (Phe), 3-(3-pyridyl)alanine (3-Pal), threonine (Thr), methionine (Met), 4-iodophenylalanine (Phe(4-I)), N6-(4-(p-tolyl)butanoyl)-lysine, N6-(4-(4-iodophenyl)butanoyl)-lysine, or γ-glutamic acid (γ-Glu);
X² is absent, asparagine (Asn), glutamine (Gln), aspartic acid (Asp), glutamic acid (Glu), serine (Ser), histidine (His), alanine (Ala), sarcosine (Sar), tyrosine (Tyr), Proline (Pro), hydroxyproline((Hyp), azetidine-2-carboxylic acid (Aze), 2,3,4,5-tetrahydroisoquinoline-3-carboxylic acid (Tic), phenylalanine(Phe), 3-(2-pyridyl)alanine (2-Pal), 3-(3-pyridyl)alanine (3-Pal), or 3-(4-pyridyl)alanine (4-Pal);
X³ is absent, tryptophan (Trp), serine (Ser), leucine (Leu), isoleucine (Ile), phenylalanine (Phe), 4-iodophenylalanine (Phe(4-1)), 3-(2-pyridyl)alanine (2-Pal), 3-(3-pyridyl)alanine (3-Pal), 3-(4-pyridyl)alanine (4-Pal), 2-amino-3-(naphthalen-2-yl)propanoic acid (H-2-NAL-OH), lysine (Lys), asparagine (Asn), glutamine (Gln), aspartic acid (Asp), glutamic acid (Glu), arginine (Arg), methyl arginine (Arg(Me)), norarginine (AGBA), methyl norarginine (AGBA(Me)), hormoarginine (HArg), methyl homoarginine (HArg(Me)), citrulline (Cit), methyl citrulline (Cit(Me)), canavanine, methyl-canavanine, glycine (Gly), alanine (Ala), sarcosine (Sar), tyrosine (Tyr), cyclohexylalanine (Cha), 3-(1-naphthyl)alanine (α-Nal), 3-(2-naphthyl)alanine (β-Nal), threonine (Thr), proline (Pro), hydroxyproline (Hyp), tetrahydroisoquinoline-3-carboxylic acid (Tic), O-phospho-serine (SOP), 2-amino-4-(2H-tetrazol-5-yl)butanoic acid, β-glutamic acid, 8-aminoquinoline-3-carboxylic acid, biphenylalanine (Bip), 4-benzoylphenylalanine (Bpa), or 3-(9-anthryl)-alanine (H-Ala(9-Anth)-OH or AAP);
X⁴ is absent, asparagine (Asn), glutamine (Gln), aspartic acid (Asp), glutamic acid (Glu), tryptophan (Trp), glycine (Gly), tyrosine (Tyr), alanine (Ala), sarcosine (Sar), or arginine (Arg);
X⁵ is absent, serine (Ser), threonine (Thr), lysine (Lys), asparagine (Asn), glutamine (Gln), aspartic acid (Asp), glutamic acid (Glu), glycine (Gly), alanine (Ala), or sarcosine (Sar);
X⁶ is absent, phenylalanine (Phe), alpha-methylphenylalanine (α-Me-Phe), N-methylphenylalanine (N-Me-Phe), 2-fluorophenylalanine (2-F-Phe), 3-fluorophenylalanine (3-F-Phe), 4-fluorophenylalanine (4-F-Phe), 4-iodophenylalanine (Phe(4-I)), 2-amino-2-indancarboxylic acid (Aic), biphenylalanine (Bip), ββ-(2-thienyl)-Ala, tryptophan (Trp), 2-aminotetralin-2-carboxylic acid (Atc), 3β-(2-thienyl)-alanine, 3-(4-pyridyl)alanine (4-Pal), cyclohexylalanine (Cha), or tyrosine (Tyr);
X⁷ is absent, glycine (Gly), aza-glycine (aza-Gly), alanine (Ala), N-methyglycine (Sar), or 1-aminocyclopropane-1-carboxylic acid (ACC);
X⁸ is leucine (Leu), norvaline (Nva), valine (Val), isoleucine (lie), homoalanine (HAla), tryptophan (Trp), phenylalanine (Phe), or phenylglycine (Phg);
or —X⁷—X⁸— is

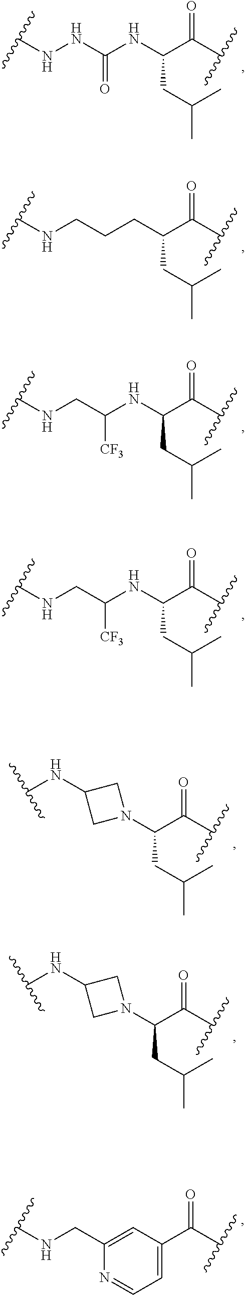

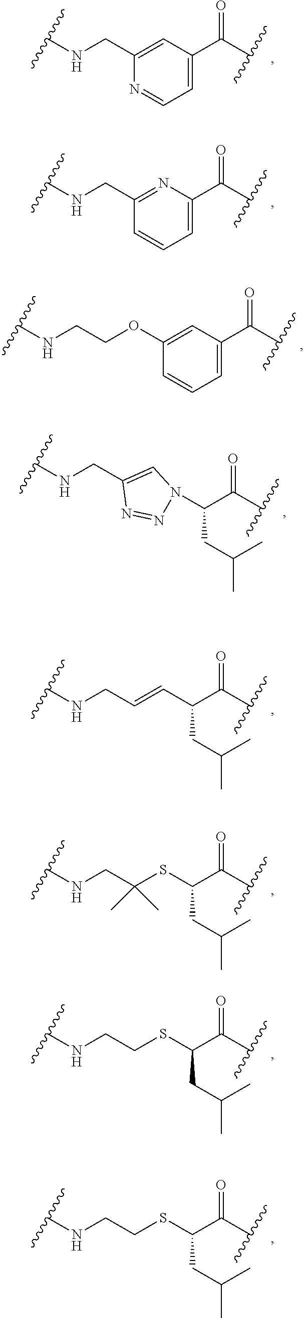

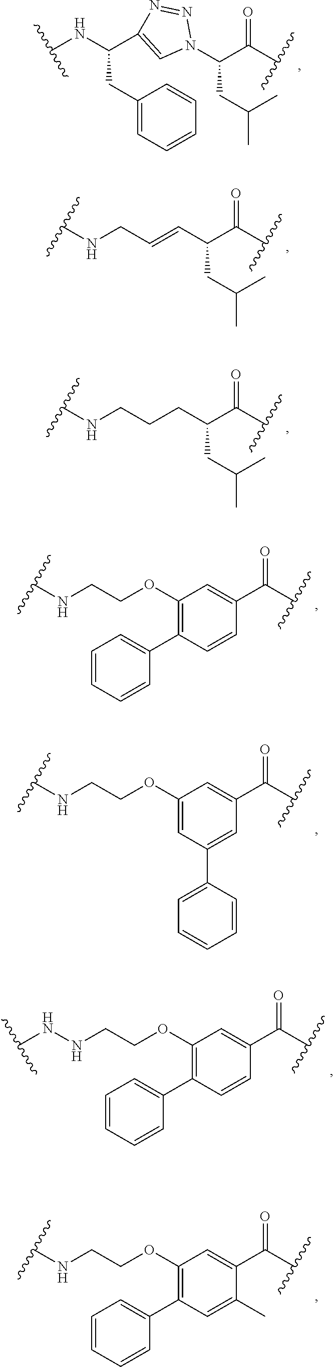

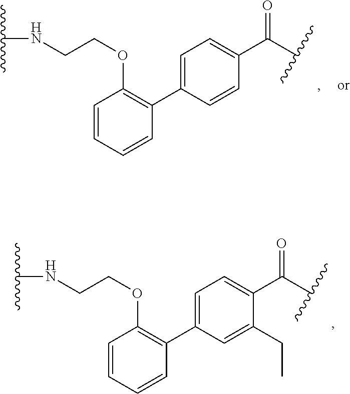 [SMILES: *NCCOc1ccccc1-c1ccc(C(*)=O)cc1 ; *NCCOc1ccccc1-c1ccc(C(*)=O)c(CC)c1]

or —X⁶—X⁷—X⁸— is

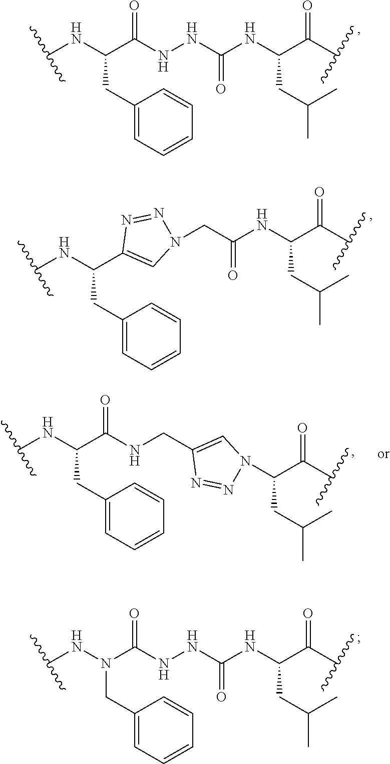 [SMILES: *N[C@@H](Cc1ccccc1)C(=O)NNC(=O)N[C@@H](CC(C)C)C(*)=O ; *N[C@@H](Cc1ccccc1)c1cn(CC(=O)N[C@@H](CC(C)C)C(*)=O)nn1 ; *N[C@@H](Cc1ccccc1)C(=O)NCc1cn([C@@H](CC(C)C)C(*)=O)nn1 ; *NN(Cc1ccccc1)C(=O)NNC(=O)N[C@@H](CC(C)C)C(*)=O]

X¹⁰ is tryptophan (Trp), 1-methyltryptophan (1MT), tyrosine (Tyr), phenylalanine (Phe), 4-cyano phenylalanine (Phe(4-CN)), 3-(4-pyridyl)alanine (4-Pal), leucine (Leu), phenylglycine (Phg), cyclohexylalanine (Cha), 3-(1-naphthyl)alanine (α-Nal), 3-(2-naphthyl)-alanine (β-Nal), histidine (His), or 3-nitro-tyrosine (Tyr(3-NO₂));
wherein the N-terminal amino acid or the compound of Formula (I) is optionally substituted with —C(═O)—C₁-C₂₀(alkyl, —C(═O)—(CH₂CH₂O)_{y}—CH₂CH₂—R¹⁵, —C₁-C₂₀alkyl, N-hexadecanoyl-Glu, —C₄-C₂₀ polyethylene glycol, a saccharide, —R¹⁶, —C(═O)—(CH₂CH₂O)ₓ—CH₃, —C(═O)—(CH₂CH₂O)ₓ—H, —C(═O)—CH₂CH₂—CH(COOH)—R¹⁵, —C(═O)—(CH₂)₂R¹⁹, or —C(═O)CH₂NHCH₂R¹⁹;
R¹⁵ is selected from —OR¹⁶, —N(R¹⁶)₂, —C(═O)OR¹⁶, or —C(═O)N(R¹⁶)₂;
each R¹⁶ is independently H, —C₁-C₆ alkyl, —C(═O)—(CH₂)R¹⁹, —C(CH₂NHCH₂R¹⁹, or a saccharide or derivative thereof;
R¹⁹ is 4-iodophenylene, 4-methylphenylene, or 3-fluoro-4-methylphenylene;
y is 0, 1, 2, 3, 4, 5, 6, 7, 8, 9, or 10; x is an integer from 1 and 25; and v is 1, 2, 3, or 4;
wherein any free—NH— of a peptide bond is optionally independently substituted with —CH₃ or —CH₂CH₃; and
wherein any alpha position of an amino acid is optionally independently substituted with —CH₃— or —CH₂CH₃.

In some embodiments, R^{a} is a chelating moiety independently selected from the group consisting of:

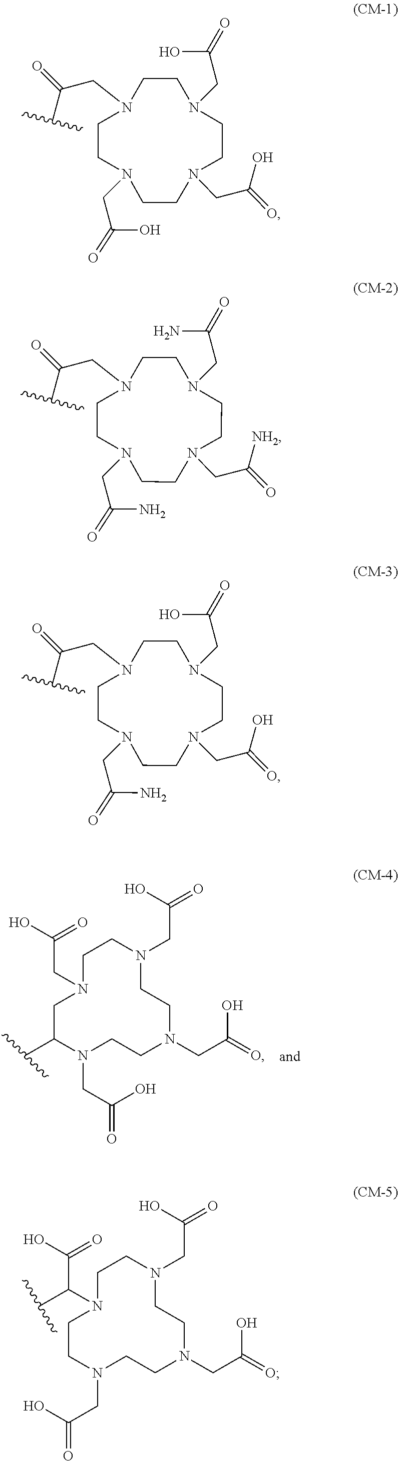 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

In some embodiments, -L- is absent, *-L¹, *—NR¹⁷-L¹-, *—NR¹⁷-L⁵-L¹-, *—NR¹⁷-L⁵-C(═O)-L¹, *—NR¹⁷-L⁵-NR¹⁷—C(═O)-L¹-, *-L⁵-C(═O)-L¹-, *-L⁵-L¹,*—NR¹⁷-L⁵-NR¹⁷-L¹-, *—NR¹⁷-L⁵-C(═O)NR¹⁷-L¹,*-(L³)_{w}, *—NR¹⁷-L⁵-C(═O)—, or *-(L³)_{w}, —NR¹⁷-L⁵-C(═O)
L¹-; wherein * denotes the attachment point to R^{a};
L⁵ is substituted or unsubstituted C₁-C₆ alkylene;
or L⁵ and R¹⁷ are taken together with the N atom to which they are attached to form N-heterocycloalkyl;
R¹⁷ is selected from hydrogen, C₁-C₆ alkyl, C₁-C₆ alkyl-CO₂H, —(CH₂CH₂O)_{z}—CH₂CH₂—CO₂H;
L¹ is absent, -L²-, -L²-(L³)_{w}-, -(L³)_{w}, -L²-(L³)_{w}, or -L²-(L³)_{w}-L²-(L³)_{w};
each L² is independently absent, —C₀-C₆ alkylene-(substituted or unsubstituted arylene)-C₀-C₆ alkylene-C(═O)—, —C₀-C₆ alkylene-(substituted or unsubstituted arylene)-C₀-C₆ alkylene-OC(═O)—, —C₀-C₆ alkylene-(substituted or unsubstituted cyclohexylene)-C₀-C₆ alkylene-C(═O)—, —C₀-C₆ alkylene-(substituted or unsubstituted heterocycloalkyl lene)-C₀-C₆alkylene-C(═O)—, —C₀-C₆alkylene-(substituted or unsubstituted heteroarylene)-C₀-C₆ alkylene-C(═O)—, —C₀-C₆ alkylene-(substituted or unsubstituted heteroarylene)-C₀-C₆ alkylene-OC(═O)—, —C₄-C₂₀ polyethylene glycol, C₄-C₂₀ polyethylene glycol-C(═O)—, substituted or unsubstituted —C₁-C₂₀ alkylene, substituted or unsubstituted C₄-C₂₀ alkylene-C(═O)—, substituted or unsubstituted 2 to 20 membered heteroalkylene, —(CH₂CH₂O)CH₂—, CH₂—, —(CH₂CH₂O)_{z}—CH₂CH₂—, (CH₂CH₂O)_{z}—CH₂—C(═O)—; or —(CH₂CH₂O)_{z}—CH₂CH₂—C(═O)—;
each z is independently 1, 2, 3, 4, 5, or 6;
each L³ is independently selected from natural or unnatural amino acids, wherein any free amine of an amino acid or peptide bond is optionally independently substituted with L⁴, and wherein when two or more amino acids are present then the N atom of the amide linking the amino acids is optionally substituted with —CH₃;
each L⁴ is independently selected from —C₁-C₆ alkyl, —C(═O)—C₁-C₆ alkyl-C(═O)—, —CO(═O)—NH—C₁-C₁ alkyl-C(═O)—, —C(═O)—C₁-C₁ alkyl-(substituted or unsubstituted heteroaryl)-C₁-C₆ alkyl-C(═O)—, —C₁-C₆ alkyl-C(═O)—, —C(═O)—C₁-C₆ alkyl-(substituted or unsubstituted aryl)-, and —C₁-C₆alkyl-(substituted or unsubstituted aryl)-C(═O)—; wherein if L⁴ is present then: L⁴ is attached to the any one of X¹, X², X³, X⁴, X⁵, X⁶, or X⁷, or L⁴ is attached to X⁸ if X¹, X², X³, X⁴, X⁵, X⁶, and X⁷ are absent, and each w is independently 1, 2, 3, 4, 5, or 6.

In some embodiments, the radionuclide of the radionuclide complex is: an Auger electron-emitting radionuclide; or an α-emitting radionuclide; or a β-emitting radionuclide; or a γ-emitting radionuclide.

Also described herein is a pharmaceutical composition comprising a compound described herein (e.g., a compound of Formula (I), or a pharmaceutically acceptable salt thereof, and at least one pharmaceutically acceptable excipient. In some embodiments, the pharmaceutical composition is formulated for administration to a mammal by intravenous administration or subcutaneous administration. In some embodiments, the pharmaceutical composition is formulated for administration to a mammal by intravenous administration.

In another aspect, described herein is a method for the treatment of cancer comprising administering to a mammal with cancer an effective amount of a compound described herein (e.g., a compound of Formula (I)), or a pharmaceutically acceptable salt thereof, or an effective amount of pharmaceutical composition comprising a compound described herein (e.g., a compound of Formula (I)), or a pharmaceutically acceptable salt thereof. In some embodiments, the cancer comprises tumors and the tumors overexpress the Kisspeptin receptor (KISS1R). In some embodiments, the cancer is glioma, thyroid cancer, lung cancer, colorectal cancer, stomach cancer, liver cancer, pancreatic cancer, renal cancer, prostate cancer, testis cancer, breast cancer, cervical cancer, endometrial cancer, ovarian cancer or melanoma. In some embodiments, the cancer is breast cancer. In some embodiments, the cancer is renal cancer. In some embodiments, the cancer is lung cancer.

In another aspect, described herein is a method for treating tumors in a mammal with a radionuclide comprising administering to the mammal a compound described herein (e.g., a compound of Formula (I)), or a pharmaceutically acceptable salt thereof, or a pharmaceutical composition comprising a compound described herein (e.g., a compound of Formula (I)), or a pharmaceutically acceptable salt thereof. In some embodiments, the mammal has been diagnosed with breast cancer. In some embodiments, the mammal has been diagnosed with renal cancer. In some embodiments, the mammal has been diagnosed with lung cancer.

In another aspect, described herein is a method of targeting delivery of a radionuclide to tumors in a mammal comprising administering to a mammal with tumors a compound described herein (e.g., a compound of Formula (I)), or a pharmaceutically acceptable salt thereof, or a pharmaceutical composition comprising a compound described herein (e.g., a compound of Formula (I)), or a pharmaceutically acceptable salt thereof; wherein the tumors overexpress the Kisspeptin receptor (KISS1R).

In another aspect, described herein is a method for identifying tissues or organs in a mammal with tumors expressing the Kisspeptin receptor (KISS1R) comprising administering to the mammal a compound described herein (e.g., a compound of Formula (I)), or a pharmaceutically acceptable salt thereof, or a pharmaceutical composition comprising a compound described herein (e.g., a compound of Formula (I)), or a pharmaceutically acceptable salt thereof; and performing positron emission tomography (PET) analysis, single-photon emission computerized tomography (SPECT), or magnetic resonance imaging (MIR); wherein R^{a} is a chelating moiety-diagnostic radionuclide complex.

In yet another aspect, described herein is a method for the in vivo imaging of tissues or organs in mammal with tumors expressing the Kisspeptin receptor (KISS1R) comprising administering to the mammal a compound described herein (e.g., a compound of Formula (I)), or a pharmaceutically acceptable salt thereof, or a pharmaceutical composition comprising a compound described herein (e.g., a compound of Formula (I)), or a pharmaceutically acceptable salt thereof; and performing positron emission tomography (PET) analysis, single-photon emission computerized tomography (SPECT), or magnetic resonance imaging (MRI); wherein R^{a} is a chelating moiety-diagnostic radionuclide complex.

In any of the embodiments disclosed herein, the mammal is a human.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

FIG. 1 depicts biodistribution of ¹¹¹In[In]-Compound 1 in tumor bearing Swiss nude mice. Timepoints are 0.5, 2.0, 5.0, 24 and 72 h post IV treatment. Activity is measured as percentage of injected dose per gram of tissue (% ID/g).

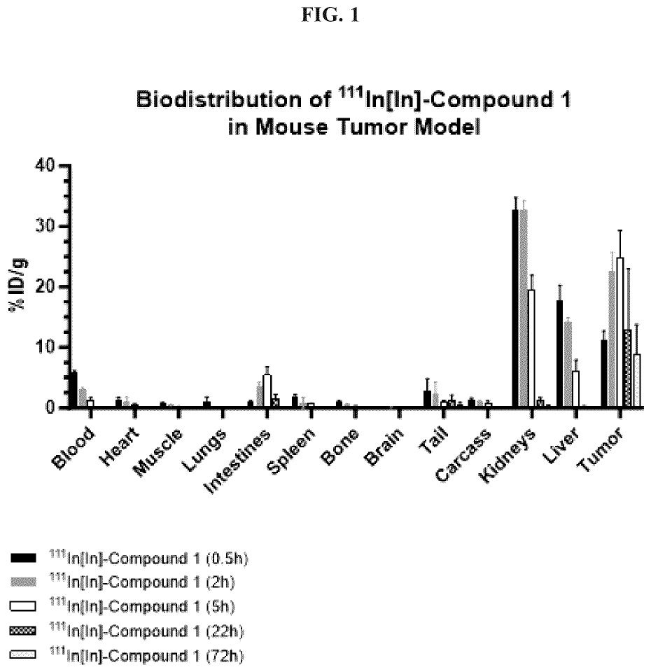

FIG. 2 depicts competition study of ¹¹¹In[In]-Compound 1 co-administered with excessive unlabeled Compound 1 (1:100 ratio respectively) in tumor bearing Swiss nude mice. Timepoint is 2 h. Activity is measured as percentage of injected dose per gram of tissue (% ID/g).

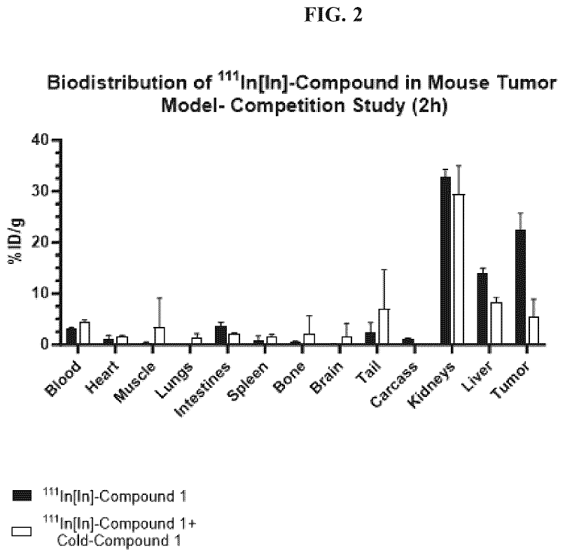

FIG. 3 depicts biodistribution of ¹¹¹In[In]-Compound 6 in tumor bearing Swiss nude mice. Timepoints are 0.75, 2.0, 5.0, 24 and 44 h post IV treatment. Activity is measured as percentage of injected dose per gram of tissue (% ID/g).

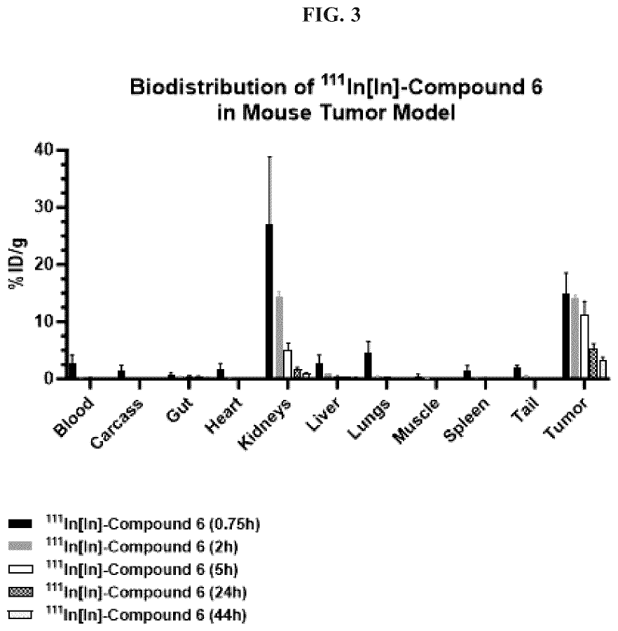

FIG. 4 depicts competition study of ¹¹¹In[In]-Compound 6 co-administered with excessive unlabeled Compound 6 (1:100 ratio respectively) in tumor bearing Swiss nude mice. Timepoint is 2 h. Activity is measured as percentage of injected dose per gram of tissue (% ID/g).

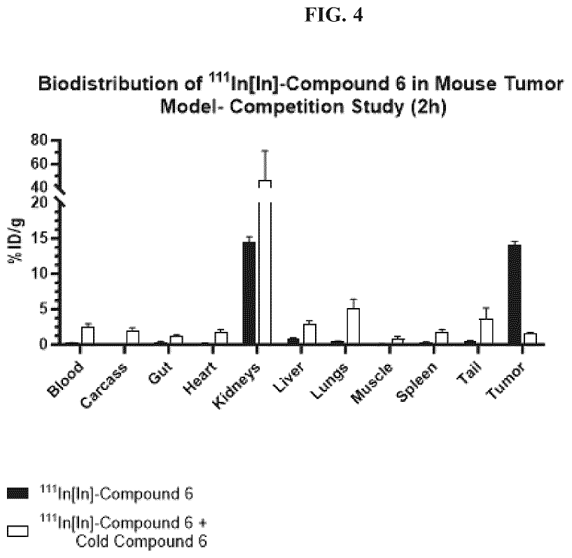

FIG. 5 depicts biodistribution of ¹¹¹In[In]-Compound 105 in tumor bearing Swiss nude mice. Timepoints are 0.5, 2.0, 5.0, 24 and 44 h post IV treatment. Activity is measured as percentage of injected dose per gram of tissue (% ID/g).

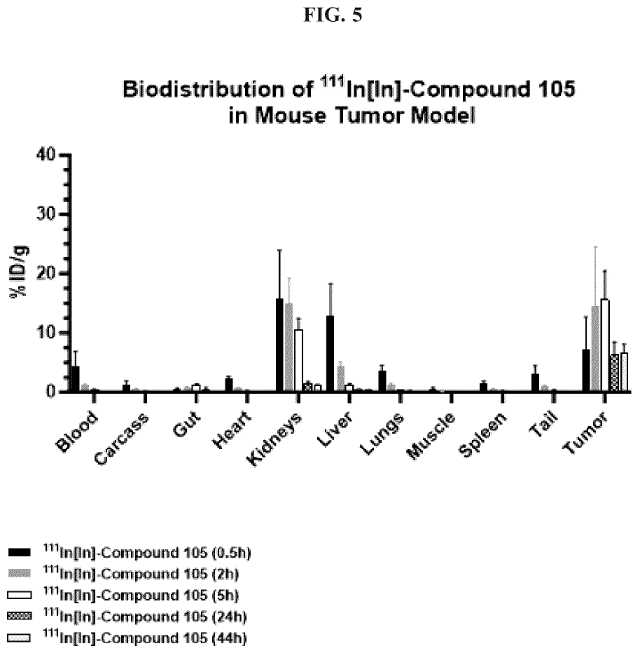

FIG. 6 depicts competition study of ¹¹¹In[In]-Compound 1 co-administered with excessive unlabeled Compound 1 (1:100 ratio respectively) in tumor bearing Swiss nude mice. Timepoints is 2 h. Activity is measured as percentage of injected dose per gram of tissue (% ID/g).

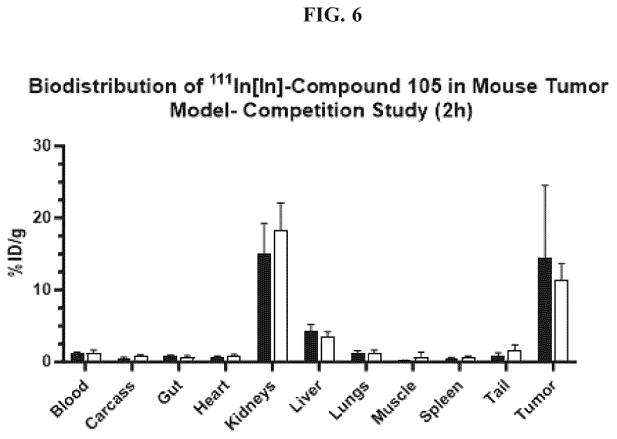

### DETAILED DESCRIPTION OF THE INVENTION

Cancer, a disease in which some cells undergo a genetic change in the control of their growth and replication that results in uncontrolled growth and spreading, is one of the leading causes of death worldwide. General types of cancers include solid tumors (cancers that typically originate in organs), carcinomas (cancers that originate in skin or tissues that line organs), sarcomas (cancers of connective tissues such as bones), leukemias cancers of bone marrow), and lymphomas and myelomas (cancers of the immune system). Neoplasms are abnormal growth of cells that result in solid tumors which may be benign (i.e. do not display malignant features and are generally unlikely to become dangerous such as adenomas), malignant (i.e. display features such as genetic mutations, loss of normal function, rapid division, and ability metastasize (invade) to other tissues), and of uncertain or unknown behavior. State-of-the-art treatment of neoplasms is accomplished by a combination of surgical procedures, chemotherapy, and radiation therapy. Surgical procedures can be curative under some conditions, but often require multiple interventions and are often done in combination with radiation and chemotherapy. Chemotherapy proves to be a potent weapon in the fight against cancer in many cases. Chemotherapy is typically performed by systemic administration of potent cytotoxic drugs, but these compounds often lack tumor selectivity and therefore also kill healthy cells in the body. The resulting non-specific toxicity is the cause of severe side effects of chemotherapy which occur because chemotherapy does not target the cancerous cells specifically over other cells. Radiotherapy is the use of high-energy radiation to kill cells. The source of radiation may be external-beam radiation (applied using an external source), internal radiation (placement of a radioactive material near the target cells), or radiotherapy from the systemic administration of a radioactive material. Like chemotherapy, many radiation therapy options also lack tumor cell identification properties needed to achieve the ultimate goal of targeted tumor therapy with drug molecules or radionuclides.

Described herein are radiopharmaceuticals that selectively deliver radionuclides to malignant cells that overexpress KISS1R for use in cancer detection, image guided cancer surgery, and selective tumor killing.

### The Kisspeptin Receptor (KISS1R)

Kisspeptin (KP) is a peptide hormone cleaved from a 145 amino acid precursor protein (KiSS1) encoded by the KiSS1 gene. Kisspeptin is made up of 54 amino acids that can be proteolytically processed into shorter peptides with a common C-terminal decapeptide sequence: Tyr-Asn-Trp-Asn-Ser-Phe-Gly-Leu-Arg-Phe-NH, (SEQ ID NO: 828). This sequence strongly binds to a G-protein coupled receptor GPR54, also known as Kisspeptin receptor (KISS1R). The KP/KISS1R signaling system has been shown to exhibit dual roles in cancer; that is, the KiSS1 gene has been reported as a metastasis promoter and suppressor, depending on the type of cancer.

Kisspeptin and its receptor are expressed in several tissues, including the brain, pancreas, placenta, and testis. KISS1R is a G-protein coupled seven transmembrane receptor. Binding of kisspeptin to KISS1R activates G-protein Gq/11 and phospholipase C to hydrolyze phosphatidylinositol-4,5-bisphosphate (PIP2) into inositol 1,4,5-triphosphate (IP3) and diacylglycerol (DAG). IP3 activates intracellular calcium release and DAG activates the mitogen-activated protein kinase (MAPK) pathway. There are several downstream effects of these signals, including effects on hormone secretion, metastasis, migration, angiogenesis, and proliferation.

The KP/KISS1R signaling system has been suggested to promote metastasis in breast cancer and liver cancer, and suppress metastasis in bladder cancer, ovarian cancer, colorectal cancer, pancreatic cancer, prostate cancer, lung cancer, and thyroid cancer. The KP/KISS1R signaling system has also been described as an important modulator of gonadotropin-releasing hormone (GnRH), a key regulator of the human reproductive system. Peptide analogs of kisspeptin have been shown to interrupt kisspeptin signaling and suppress the pulsatile secretion of GnRH, showing promise for treating hormone-dependence diseases such as prostate cancer. These peptide analogs show evidence of higher metabolic stability than native kisspeptins and also display good KISS1R agonist activity. Radiopharmaceuticals targeting KISS1R are important for the development of new cancer therapies.

### Breast Cancer

Breast cancer is a type of cancer that starts in the breast. It can start in one or both breasts, in various parts of the breast. There are many types of breast cancer, and a breast cancer's type is determined by the specific cells in the breast that become cancer.

### Breast Cancer Types

Most breast cancers are carcinomas, which are tumors that start in the epithelial cells that line organs and tissues throughout the body. When carcinomas form in the breast, they are usually a more specific type called adenocarcinoma, which starts in cells in the ducts (the milk ducts) or the lobules (glands in the breast that make milk).

The type of breast cancer can also refer to whether the cancer has spread or not. In situ breast cancer (ductal carcinoma in situ or DCIS) is a pre-cancer that starts in a milk duct and has not grown into the rest of the breast tissue. The term invasive (or infiltrating) breast cancer is used to describe any type of breast cancer that has spread (invaded) into the surrounding breast tissue.

### Breast Cancer Staging

The staging system most often used for breast cancer is the American Joint Committee on Cancer (AJCC) TNM system. The most recent AJCC system, effective January 2018, has both clinical and pathologic staging systems for breast cancer:

The pathologic stage (also called the surgical stage) is determined by examining tissue removed during an operation.

Sometimes, if surgery is not possible right away or at all, the cancer will be given a clinical stage instead. This is based on the results of a physical exam, biopsy, and imaging tests. The clinical stage is used to help plan treatment. Sometimes, though, the cancer has spread further than the clinical stage estimates, and may not predict the patient's outlook as accurately as a pathologic stage.

In both staging systems, 7 key pieces of information are used:
i. The extent (size) of the tumor (T);
ii. The spread to nearby lymph nodes (N);
iii. The spread (metastasis) to distant sites (M);
iv. Estrogen Receptor (ER) status;
v. Progesterone Receptor (PR) status;
vi. HER2 status; and vii. Grade of the cancer (G).

In addition, Oncotype Dx® Recurrence Score results may also be considered in the stage in certain situations. Once all of these factors have been determined, this information is combined in a process called stage grouping to assign an overall stage.

### Breast Cancer Treatment

Tumors can form in the breasts. The types of treatment used to treat breast tumors include: surgery, radiation therapy, chemotherapy, hormone therapy, targeted drug therapy and immunotherapy.

There are two main types of surgery to remove breast cancer: breast-conserving surgery and mastectomy. Breast-conserving surgery is surgery to remove the cancer as well as some surrounding normal tissue. Only the part of the breast containing the cancer is removed. How much breast is removed depends on where and how big the tumor is, as well as other factors. This surgery is also called a lumpectomy, quadrantectomy, partial mastectomy, or segmental mastectomy. Mastectomy is a surgery in which the entire breast is removed, including all of the breast tissue and sometimes other nearby tissues. There are several different types of mastectomies. Some women may also have both breasts removed in a double mastectomy. Sometimes surgery is done to remove the nearby lymph nodes and other tissue where the cancer has spread.

Radiation therapy uses high-energy x-rays or other types of radiation to kill cancer cells or keep them from growing. There are two types of radiation therapy: external radiation therapy uses a machine outside the body to send radiation toward the area of the body with cancer; internal radiation therapy uses a radioactive substance sealed in needles, seeds, wires, or catheters that are placed directly into or near the cancer. Additionally, targeted radiopharmaceuticals can provide targeted radiation to the site of the tumor. Chemotherapy is a cancer treatment that uses drugs to stop the growth of cancer cells, either by killing the cells or by stopping them from dividing.

Thus, a need exists for treatment options for breast tumors. Described herein are radiopharmaceuticals that target delivery of radionuclides to breast tumors, which overexpress the KISS1R. Targeted therapies usually cause less harm to normal cells than chemotherapy or radiation therapy do.

Solid tumors: benign and/or malignant neoplasms (cancer)

In one aspect, the KISS1R-targeted radiopharmaceuticals described herein are used to treat benign and/or malignant neoplasms (solid tumors), wherein the neoplasm comprises cells that overexpress KISS1R on the cell surface.

The term “neoplasm” as used herein, refers to an abnormal growth of cells that may proliferate in an uncontrolled way and may have the ability to metastasize (spread).

Neoplasms include solid tumors, adenomas, carcinomas, sarcomas, leukemias and lymphomas, at any stage of the disease with or without metastases.

A solid tumor is an abnormal mass of tissue that usually does not contain cysts or liquid areas. Solid tumors may be benign (not cancer), or malignant (cancer). Different types of solid tumors are named for the type of cells that form them. Examples of solid tumors are sarcomas, carcinomas, and lymphomas. Leukemias (cancers of the blood) generally do not form solid tumors.

Solid tumors are cancers that typically originate in organs, such as the bladder, bowel, brain, breast, endometrium, heart, kidney, lung, liver, uterus, ovaries, pancreas or other endocrine organs (thyroid), and prostate.

In some embodiments, the KISS1R-targeted radiopharmaceuticals described herein are used to treat an adenoma. An adenoma is a tumor that is not cancer. It starts in gland-like cells of the epithelial tissue (thin layer of tissue that covers organs, glands, and other structures within the body). An adenoma can grow from many glandular organs, including the adrenal glands, pituitary gland, thyroid, prostate, and others Even though benign, they have the potential to cause serious health complications by compressing other structures (mass effect) and by producing large amounts of hormones in an unregulated, non-feedback-dependent manner (causing paraneoplastic syndromes). Overtime adenomas may transform to become malignant, at which point they are called adenocarcinomas.

Adenomas may be found in the colon (e.g. adenomatous polyps, which have a tendency to become malignant and to lead to colon cancer), kidneys (e.g. renal adenomas may be precursor lesions to renal carcinomas), adrenal glands (e.g. adrenal adenomas; some secrete hormones such as cortisol, causing Cushing's syndrome, aldosterone causing Conn's syndrome, or androgens causing hyperandrogenism), thyroid (e.g. thyroid adenoma), pituitary (e.g. pituitary adenomas, such as prolactinoma, Cushing's disease and acromegaly), parathyroid (e.g. an adenoma of a parathyroid gland may secrete inappropriately high amounts of parathyroid hormone and thereby cause primary hyperparathyroidism), liver (e.g. hepatocellular adenoma), breast (e.g. fibroadenomas), appendix (e.g. cystadenoma), bronchial (e.g. bronchial adenomas may cause carcinoid syndrome, a type of paraneoplastic syndrome), prostate (e.g. prostate adenoma), sebaceous gland (e.g. sebaceous adenoma), and salivary glands.

Metastasis is the spread of malignant cells to new areas of the body, often by way of the lymph system or bloodstream. A metastatic tumor is one that has spread from the primary site of origin, or where it started, into different areas of the body. Metastatic tumors comprise malignant cells that may express cell surface KISS1R.

Tumors formed from cells that have spread are called secondary tumors. Tumors may have spread to areas near the primary site, called regional metastasis, or to parts of the body that are farther away, called distant metastasis.

In some embodiments, the tumor to be treated comprises tumor cells expressing KISS1R, wherein the tumor is a primary or metastatic tumor. In some embodiments, the tumor to be treated comprises tumor cells expressing KISS1R, wherein the tumor is a primary or metastatic tumor of breast origin. In some embodiments, the tumor to be treated comprises tumor cells expressing KISS1R, wherein the tumor is a primary or metastatic tumor of endometrial origin. In some embodiments, the tumor to be treated comprises tumor cells expressing KISS1R, wherein the tumor is a primary or metastatic tumor of ovarian origin. In some embodiments, the tumor to be treated comprises tumor cells expressing KISS1R, wherein the tumor is a primary or metastatic tumor of prostate origin. In some embodiments, the tumor to be treated comprises tumor cells expressing KISS1R, wherein the tumor is a primary or metastatic tumor of renal origin. In some embodiments, the tumor to be treated comprises tumor cells expressing KISS1R, wherein the tumor is a primary or metastatic tumor of lung origin.

In some embodiments, the KISS1R-targeted radiopharmaceuticals described herein are used to treat a carcinoma. Carcinomas include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, etc. In some embodiments, the KISS1R-targeted radiopharmaceuticals described herein are used to treat breast carcinoma. In some embodiments, the KISS1R-targeted radiopharmaceuticals described herein are used to treat renal cell carcinoma. In some embodiments, the KISS1R-targeted radiopharmaceuticals described herein are used to treat lung carcinoma.

In some embodiments, the KISS1R-targeted radiopharmaceuticals described herein are used to treat a sarcoma. Sarcomas include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Solid tumors include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. Benign solid tumors include adenomas.

Primary and metastatic tumors include, e.g., lung cancer (including, but not limited to, lung adenocarcinoma, squamous cell carcinoma, large cell carcinoma, bronchioloalveolar carcinoma, non-small-cell carcinoma, small cell carcinoma, mesothelioma); breast cancer (including, but not limited to, ductal carcinoma, lobular carcinoma, inflammatory breast cancer, clear cell carcinoma, mucinous carcinoma); colorectal cancer (including, but not limited to, colon cancer, rectal cancer); anal cancer; pancreatic cancer (including, but not limited to, pancreatic adenocarcinoma, islet cell carcinoma, neuroendocrine tumors); prostate cancer; ovarian carcinoma (including, but not limited to, ovarian epithelial carcinoma or surface epithelial-stromal tumor including serous tumor, endometrioid tumor and mucinous cystadenocarcinoma, sex-cord-stromal tumor); liver and bile duct carcinoma (including, but not limited to, hepatocellular carcinoma, cholangiocarcinoma, hemangioma); esophageal carcinoma (including, but not limited to, esophageal adenocarcinomna and squamous cell carcinoma); non-Hodgkin's lymphoma; bladder carcinoma; carcinoma of the uterus (including, but not limited to, endometrial adenocarcinoma, uterine papillary serous carcinoma, uterine clear-cell carcinoma, uterine sarcomas and leiomyosarcomas, mixed mullerian tumors); glioma, glioblastoma, medulloblastoma, and other tumors of the brain; kidney cancers (including, but not limited to, renal cell carcinoma, clear cell carcinoma, Wilm's tumor); cancer of the head and neck (including, but not limited to, squamous cell carcinomas); cancer of the stomach (including, but not limited to, stomach adenocarcinomna, gastrointestinal stromal tumor); multiple myeloma; testicular cancer; germ cell tumor; neuroendocrine tumor; cervical cancer; carcinoids of the gastrointestinal tract, breast, and other organs; and signet ring cell carcinoma.

### Representative Kisspeptin Receptor (KISS1R) Targeting Conjugates

In some embodiments, the KISS1R-targeted radiopharmaceuticals described herein have an affinity to KISS1R that is at least 10-fold, at least 50-fold, at least 100-fold, at least 200-fold, at least 500-fold, or at least 1000-fold greater than the affinity for other non-target receptors.

In some embodiments, the KISS1R-targeted radiopharmaceuticals described herein preferentially accumulate in tumor tissues that express the targeted KISS1R. In some embodiments, the KISS1R-targeted radiopharmaceuticals described herein preferentially accumulate in tissues or organs comprising tumor cells that express KISS1R as compared to tissues or organ(s) lacking tumor cells that express KISS1R. In some embodiments, the KISS1R-targeted radiopharmaceuticals described herein preferentially accumulate at least 1-fold, at least 2-fold, at least 3-fold, at least 4-fold, at least 5-fold, or greater than 5-fold more in tissues or organ(s) comprising tumor cells that express KISS1R as compared to tissues or organs lacking tumor cells that express KISS1R. It is understood that the compound may accumulate in certain tissues and organs involved in the metabolism and or excretion of therapeutics, including but not limited to the kidneys and liver.

In one aspect, described herein is a compound of Formula (I), or a pharmaceutically acceptable salt thereof:

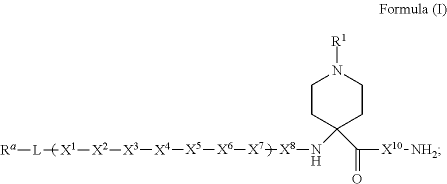

wherein:
R^{a} is a chelating moiety or a radionuclide complex thereof;
L is an optional linker that is attached to any one of X¹, X², X³, X⁴, X⁵, X⁶, or X⁷; or L is attached to X⁸ if X¹, X², X³, X⁴, X⁴, X⁶, and X⁷ are absent;
R¹ is H,

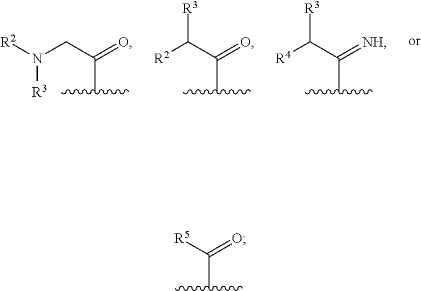

R² is C₁-C₆ alkyl, substituted or unsubstituted heteroalkyl, —(CHR⁶)ₙ-heterocycloalkyl, —(CHR⁶)ₙ-aryl, —(CHR⁶)ₙ-heteroaryl, —C(═O)—(CHR⁶)ₙ-aryl, or —C(═O)NH—(CHR⁶)ₙ-aryl; wherein C₁-C₆ alkyl is optionally substituted with R⁷, and wherein the heterocycloalkyl, aryl, or heteroaryl are each independently optionally substituted with R⁷, R⁸, R⁹, R¹⁰, and
R³ is H or C₁-C₄ alkyl;
R⁴ is H, C₁-C₄ alkyl, or R²;
R⁵ is substituted or unsubstituted alkyl, substituted or unsubstituted heteroalkyl, or —(CHR⁶)ₙ-aryl; wherein aryl is optionally substituted with R⁷, R⁸, R⁹, R¹⁰, and R¹¹;
each R⁶ is independently H, F, —CH₃, —NH₂, or —OH;
R⁷, R⁸, R⁹, R¹⁰, and R¹¹ are each independently selected from H, F, Cl, Br, I, —OH, —O—C₁-C₄ alkyl, —NH₂, —NHC₁-C₄ alkyl, —N(C₁-C₄ alkyl)₂, —CN, —CO₂H, —CO₂C₁-C₄ alkyl, —C₁-C₆ alkyl, —C₁-C₆ fluroroalkyl or —C₆-C₆ cycloalkyl;
n is 0, 1, 2, 3, 4, 5, or 6;
X¹ is absent, tyrosine (Tyr), glycine (Gly), sarcosine (Sar), alanine (Ala), aspartic acid (Asp), lysine (Lys), phenylalanine (Phe), or D-3-(3-pyridyl)alanine (D-3-Pal), threonine (Thr), methionine (Met), 4-iodophenylalanine (Phe(4-I)), N6-(4-(p-tolyl)butanoyl)-lysine, or N6-(4-(4-iodophenyl)butanoyl)-lysine, or γ-glutamic acid (γ-Glu);
X² is absent, asparagine (Asn), glutamine (Gln), aspartic acid (Asp), glutamic acid (Glu), serine (Ser), histidine (His), alanine (Ala), sarcosine (Sar), tyrosine (Tyr), proline (Pro), hydroxyproline(Hyp), azetidine-2-carboxylic acid (Aze), 2,3,4,5-tetrahydroisoquinoline-3-carboxylic acid (Tic), phenylalanine(Phe), 3-(2-pyridyl)alanine (2-Pal), 3-(3-pyridyl)alanine (3-Pal), or 3-(4-pyridylalanine (4-Pal);
X³ is absent, tryptophan (Trp), serine (Ser), leucine (Leu), isoleucine (Ile), phenylalanine (Phe), 4-iodophenylalanine(Phe(4-4)), 3-(2-pyridyl)alanine (2-Pal), 3-(3-pyridyl)alanine (3-Pal), 3-(4-pyridyl)alanine (4-Pal), 2-amino-3-(naphthalen-2-yl)propanoic acid (1H-2-NAL-OH), lysine (Lys), asparagine (Asn), glutamine (Gln), aspartic acid (Asp), glutamic acid (Glu), arginine (Arg), methyl arginine (Arg(Me)), norarginine (AGBA), methyl norarginine (AGBA(Me)), homoarginine (HArg), methyl homoarginine (HArg(Me)), citrulline (Cit), methyl citrulline (Cit(Me)), canavanine, methyl-canavanine, glycine (Gly), alanine (Ala), sarcosine (Sar), tyrosine (Tyr), cyclohexylalanine (Cha), 3-(1-naphthyl)alanine (α-Nal), 3-(2-naphthyl)alanine (β-Nal), threonine (Thr), proline (Pro), hydroxyproline (Hyp), tetrahydroisoquinoline-3-carboxylic acid (Tic), O-phospho-serine (SOP), 2-amino-4-(2H-tetrazol-5-yl)butanoic acid, β-glutamic acid, 8-aminoquinoline-3-carboxylic acid, biphenylalanine (Bip), 4-benzoylphenylalanine (Bpa), or 3-(9-anthryl)-alanine (H-Ala(9-Anth)-OH or AAP);
X¹ is absent, asparagine (Asn), glutamine (Gln), aspartic acid (Asp), glutamic acid (Glu), tryptophan (Trp), glycine (Gly), tyrosine (Tyr), alanine (Ala), sarcosine (Sar), or arginine (Arg);
X⁵ is absent, serine Ser), threonine (Thr), lysine (Lys), asparagine (Asn), glutamine (Gln), aspartic acid (Asp), glutamic acid (Glu), glycine (Gly), alanine (Ala), or sarcosine (Sar);
X⁶ is absent, phenylalanine (Phe), alpha-methylphenylalanine (α-Me-Phe), N-methylphenylalanine (N-Me-Phe), 2-fluorophenylalanine (2-F-Phe), 3-fluorophenylalanine (3-F-Phe), 4-fluorophenylalanine (4-F-Phe), 4-iodophenylalanine (Phe(4-I)), 2-amino-2-indancarboxylic acid (Aic), biphenylalanine (Bip), (ββ-(2-thienyl)-Ala), tryptophan (Trp), 2-aminotetralin-2-carboxylic acid (Atc), 3β-(2-thienyl)-alanine, 3-(4-pyridyl)alanine (4-Pal), cyclohexylalanine (Cha), or tyrosine (Tyr);
X⁷ is absent, glycine (Gly), aza-glycine (aza-Gly), alanine (Ala), N-methylglycine (Sar), or 1-aminocyclopropane-1-carboxylic acid (ACC);
X⁸ is leucine (Leu), norvaline (Nva) valine (Val) isoleucine (Lie), homoalanine (HAla), tryptophan (Trp), phenylalanine (Phe), or phenylglycine (Phg);
or —X⁷—X⁸— is

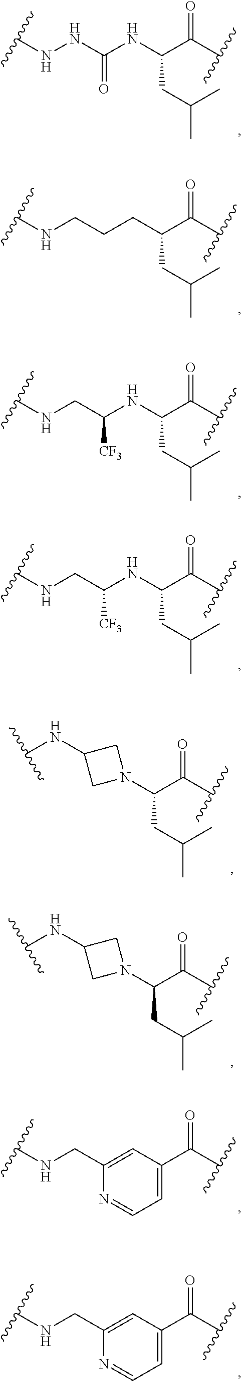

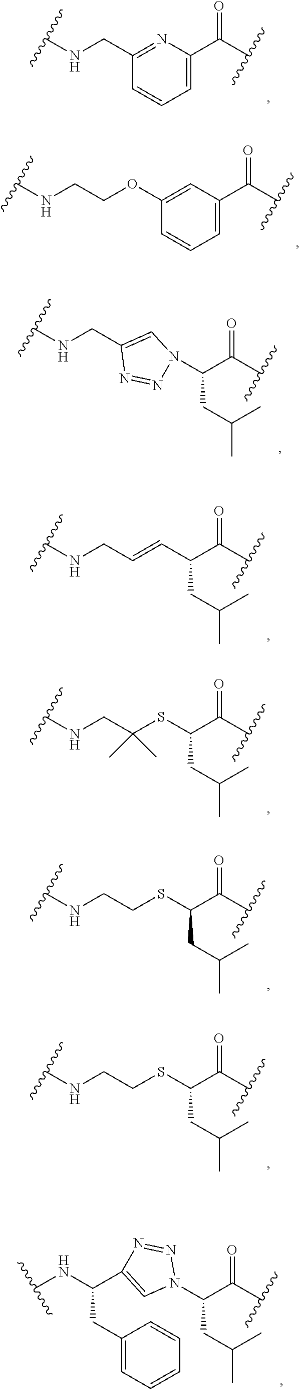

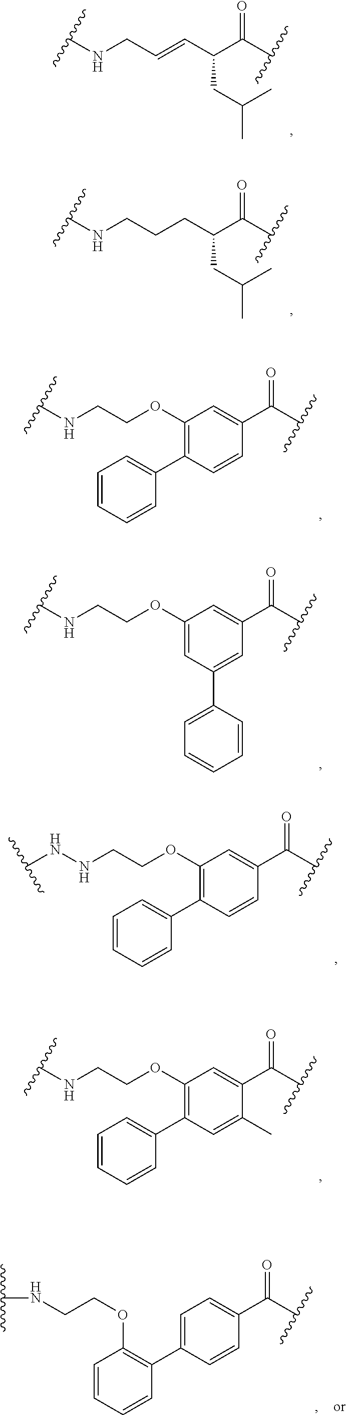

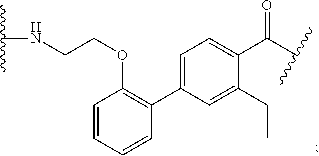 [SMILES: *NCCOc1ccccc1-c1ccc(C(*)=O)c(CC)c1]

or —X⁶—X⁷—X⁸— is

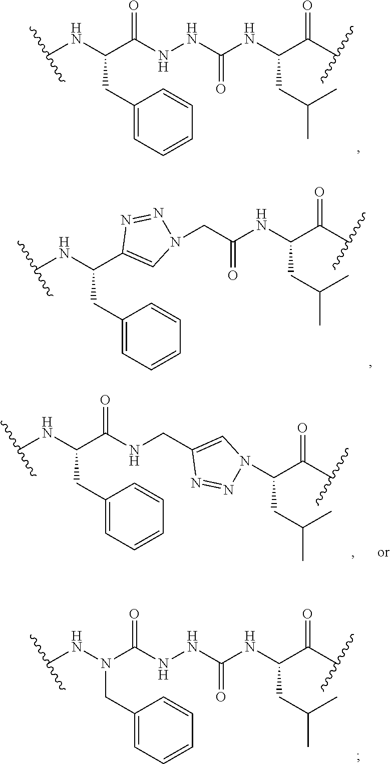 [SMILES: *N[C@@H](Cc1ccccc1)C(=O)NNC(=O)N[C@@H](CC(C)C)C(*)=O ; *N[C@@H](Cc1ccccc1)c1cn(CC(=O)N[C@@H](CC(C)C)C(*)=O)nn1 ; *N[C@@H](Cc1ccccc1)C(=O)NCc1cn([C@@H](CC(C)C)C(*)=O)nn1 ; *NN(Cc1ccccc1)C(=O)NNC(=O)N[C@@H](CC(C)C)C(*)=O]

X¹⁰ is tryptophan (Trp), 1-methyltryptophan (1 MT) tyrosine (Tyr), phenylalanine (Phe), 4-cyano phenylalanine (Phe(4-CN)), 3-(4-pyridyl)alanine (4-Pal), leucine (Leu), phenylglycine (Phg), cyclohexylalanine (Cha), 3-(1-naphthyl)alanine (α-Nal), 3-(2-naphthyl)-alanine (β-Nal), histidine (His), or 3-nitro-tyrosine (Tyr(3-NO₂));
   wherein the N-terminal amino acid or the compound of Formula (I) is optionally substituted with —C(═O)—C₁-C₂₀ alkyl, —C(═O)—(CH₂CH₂O)_{y}—CH₂CH₂—R¹⁵, C₁-C₂₀ alkyl, N-hexadecanoyl-Glu, C₄-C₂₀ polyethylene glycol, a saccharide, —R¹⁶, —C(═O)—(CH₂CH₂O)ₓ—CH₃, —C(═O)—(CH₂CH₂O)ₓ—H, —C(═O)—CH₂CH₂CH(COOH)—R¹⁵, —C(═O)—(CH₂)₂R¹⁹, or —C(═O))CH₂NHCH₂R¹⁹;
R¹⁵ is selected from —OR¹⁶, —N(R¹⁶)₂, —C(═O)OR¹⁶, or C(═O)N(R¹⁶)₂;
   each R¹⁶ is independently H, —C₁-C₆ alkyl, —C(═O)—(CH₂)ᵥR¹⁹, —C(═O)CH—NHCH₂R¹⁹, or a saccharide or derivative thereof; R¹⁹ is 4-iodophenylene, 4-methylphenylene, or 3-fluoro-4-methylphenylene;
y is 0, 1, 2, 3, 4, 5, 6, 7, 8, 9, or 10;
x is an integer from 1 and 25; and
v is 1, 2, 3, or 4;
wherein any free —NH— of a peptide bond is optionally independently substituted with —CH₃ or —CH₂CH₃; and
wherein any alpha position of an amino acid is optionally independently substituted with —CH₃ or —CH₂CH₃.

In some embodiments, described herein is a compound of Formula (I), or a pharmaceutically acceptable salt thereof:

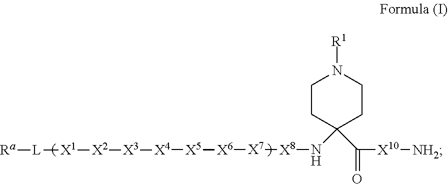

wherein:
R^{a} is a chelating moiety or a radionuclide complex thereof;
L is an optional linker that is attached to any one of X¹, X², X³, X⁴, X⁵, X⁶, or X⁷;
or L is attached to X⁸ if X¹, X², X³, X⁴, X⁵, X⁶ and X⁷ are absent; and
R¹ is H,

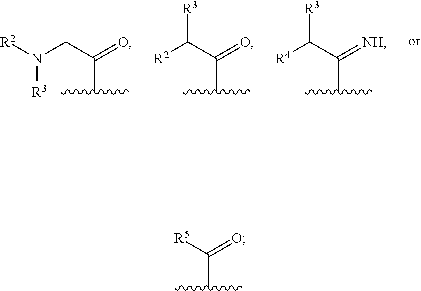

R² is C₁-C₆ alkyl, substituted or unsubstituted heteroalkyl, —(CHR⁶)ₙ-aryl, —C(═O)—(CHR⁶)ₙ-aryl, or —C(═O)NH—(CHR⁶)ₙ-aryl; wherein C₁-C₆ alkyl is optionally substituted with R⁷, and
   wherein aryl is optionally substituted with R⁷, R⁸, R⁹, R¹⁰, and R¹¹;
R³ is H or C₁-C₄ alkyl;
R⁴ is H, C₁-C₄ alkyl, or R²;
R⁵ is substituted or unsubstituted alkyl, substituted or unsubstituted heteroalkyl, or —(CHR⁶)ₙ-aryl; wherein aryl is optionally substituted with R⁷, R⁸, R⁹, R¹⁰, and R¹¹;
each R⁶ is independently H, F, —CH₃, —NH₂, or —OH;
R⁷, R⁸, R⁹, R¹⁰, and R¹¹ are independently selected from H, F, Cl, Br, I, —OH, —O—C₁-C₄ alkyl, —NH₂, —NHC₁-C₄ alkyl, —N(C₁-C₄ alkyl)₂, —CN, —CO₂, —CO₂C₁-C₄ alkyl, —C₁-C₆ alkyl, —C₁-C₆ fluoroalkyl or —C₃-C₆ cycloalkyl;
   n is 0, 1, 2, 3, 4, 5, or 6:
X¹ is absent, tyrosine (Tyr), glycine (Gly), sarcosine (Sar), alanine (Ala), aspartic acid (Asp), lysine (Lys), phenylalanine (Phe), or D-3-(3-pyridyl)alanine (D-3-Pal);
X² is absent, asparagine (Asn), glutamine (Gln), aspartic acid (Asp), glutamic acid (Glu), serine (Ser), histidine (His), alanine (Ala), sarcosine (Sar), tyrosine (Tyr), Proline (Pro), hydroxyproline(Hyp), azetidine-2-carboxylic acid (Aze), 2,3,4,5-tetrahydroisoquinoline-3-carboxylic acid (Tic), phenylalanine (Phe), 3-(2-pyridyl)alanine (2-Pal), 3-(3-pyridyl)alanine (3-Pal), or 3-(4-pyridyl)alanine (4-Pal);
X³ is absent, tryptophan (Trp), serine (Ser), leucine (Leu), isoleucine (Ile), phenylalanine (Phe), 3-(2-pyridyl)alanine (2-Pal), 3-(3-pyridyl)alanine (3-Pal), 3-(4-pyridyl)alanine (4-Pal), lysine (Lys), asparagine (Asn), glutamine (Gln), aspartic acid (Asp), glutamic acid (Glu), arginine (Arg), methyl arginine (Arg(Me)), norarginine (AGBA), methyl norarginine (AGBA(Me)), homoarginine (HArg), methyl homoarginine (HArg(Me)), citrulline (Cit), methyl citrulline (Cit(Me)), canavanine, methyl-canavanine, glycine ((Gly), alanine (Ala), sarcosine (Sar), tyrosine (Tyr), cyclohexylalanine (Cha), 3-(1-naphthyl)alanine (α-Nal), 3-(2-naphthyl)alanine (β-Nal), or threonine (Thr), proline (Pro), hydroxyproline (Hyp), or tetrahydroisoquinoline-3-carboxylic acid (Tic);
X⁴ is absent, asparagine (Asn), glutamine (Gln), aspartic acid (Asp), glutamic acid (Glu), tryptophan (Trp), glycine (Gly), tyrosine (Tyr), alanine (Ala), or sarcosine (Sar);
X⁵ is absent, serine (Ser), threonine (Thr), lysine (Lys), asparagine (Asn), glutamine (Gln), aspartic acid (Asp), glutamic acid (Glu), glycine ((Gly), alanine (Ala), or sarcosine (Sar);
X⁶ is absent, phenylalanine (Phe), alpha-methylphenylalanine (α-Me-Phe), N-methylphenylalanine (N-Me-Phe), 2-fluorophenylalanine (2-F-Phe), 3-fluorophenylalanine (3-F-Phe), 4-fluorophenylalanine (4-F-Phe), 2-amino-2-indancarboxylic acid (Aic), biphenylalanine (Bip), tryptophan (Trp), 2-aminotetralin-2-carboxylic acid (Atc);
X⁷ is absent, glycine (Gly), aza-glycine (aza-Gly), alanine (Ala), N-methylglycine (Sar), or 1-aminocyclopropane-1-carboxylic acid (ACC);
X⁸ is leucine (Leu), norvaline (Nva), valine (Val), isoleucine (Ile), homoalanine (HAla), tryptophan (Trp), phenylalanine (Phe), or phenylglycine (Phg);
or —X⁶—X⁷—X⁸— is

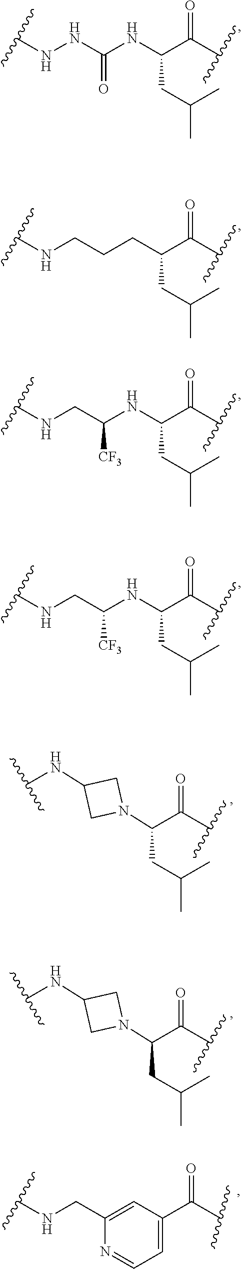

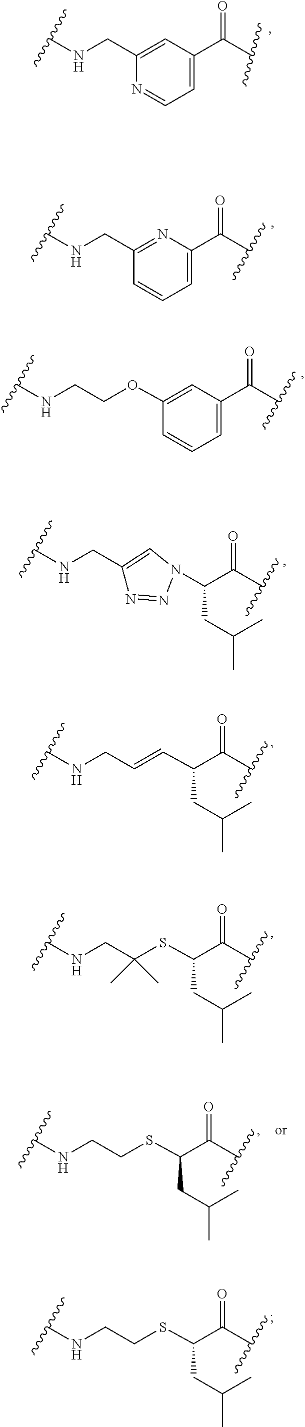

or —X⁶—X⁷—X⁸— is

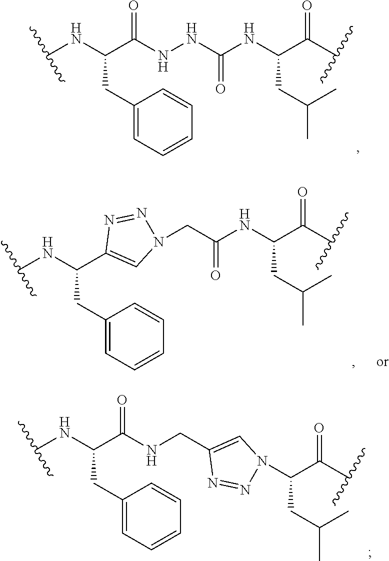

X¹⁰ is tryptophan (Trp), 1-methyltryptophan (1MT), tyrosine (Tyr), phenylalanine (Ph e), 4-cyano phenylalanine (Phe(4-CN)), 3-(4-pyridylalanine (4-Pal), leucine (Leu), phenylglycine. (Phg), cyclohexylalanine (Cha) 3-(1-naphthyl)alanine (α-Nal), 3-(2-naphthyl)-alanine (β-Nal);
wherein the N-terminal amino acid or the compound of Formula, (1) is optionally substituted with —C(═O)—C₁-C₂₀alkyl, —C(═O)—(CH₂CH₂O)_{y}—CH₂CH₂—R¹⁵, C₁-C₂₀alkyl, N-hexadecanoyl-Glu, C₄-C₂₀ polyethylene glycol, a saccharide
R¹⁵ is selected from —OR¹⁶, —N(R¹⁶)₂, —C(═O)OR¹⁶, —C(═O)N(R¹⁶)₂;
each R¹⁶ is independently H, —C₁-C₆ alkyl, or a saccharide or derivative thereof;
y is 0, 1, 2, 3, 4, 5, 6, 7, 8, 9, or 10;
wherein any free —NH— of a peptide bond is optionally independently substituted with —CH₃ or —CH₂CH₃; and
wherein any alpha position of an amino acid is optionally independently substituted with —CH₃ or —CH₂CH₃.

In some embodiments,
X¹ is absent, tyrosine (Tyr), glycine (Gly), sarcosine (Sar), alanine (Ala), aspartic acid (Asp), lysine (Lys), phenylalanine (Phe), 3-(3-pyridyl)alanine (3-Pal) (3-Pal), threonine (Thr), methionine (Met), 4-iodophenylalanine (Phe(4-1)), N6-(4-(p-tolyl)butanoyl)-lysine, N6-(4-(4-iodophenyl)butanoyl)-lysine, or γ-glutamic acid (γ-Glu);
X² is absent, asparagine (Asn), glutamine (Gln), aspartic acid (Asp), glutamic acid (Glu), serine (Ser), histidine (His), alanine (Ala), sarcosine (Sar), tyrosine (Tyr), proline (Pro), hydroxyproline(Hyp), azetidine-2-carboxylic acid (Aze), 2,3,4,5-tetrahydroisoquinoline-3-carboxylic acid (Tic), phenylalanine (Phe), 3-(2-pyridyl)alanine (2-Pal), 3-(3-pyridyl)alanine (3-Pal), or 3-(4-pyridyl)alanine (4-Pal);
X³ is absent, tryptophan (Trp), serine (Ser), leucine (Leu), isoleucine (Ile), phenylalanine (Phe), 4-iodophenylalanine(Phe(4-1)), 3-(2-pyridyl)alanine (2-Pal), 3-(3-pyridyl)alanine (3-Pal), 3-(4-pyridyl)alanine (4-Pal), 2-amino-3-(naphthalen-2-yl)propanoic acid (H-2-NAL-OH), lysine (Lys), asparagine (Asn), glutamine (Gln), aspartic acid (Asp), glutamic acid (Gi), arginine (Arg), methyl arginine (Arg(Me)), norarginine (AGBA), methyl norarginine (AGBA(Me)), homoarginine (HArg), methyl homoarginine (HArg(Me)), citrulline (Cit), methyl citrulline (Cit(Me)), canavanine, menthyl-canavanine, glycine (Gly), alanine (Ala), sarcosine (Sar), tyrosine (Tyr), cyclohexylalanine (Cha), 3-(1-naphthyl)alanine (α-Nal), 3-(2-naphthyl)alanine (β-Nal), threonine (Thr), proline (Pro), hydroxyproline (Hyp), tetrahydroisoquinoline-3-carboxylic acid (Tic), O-phospho-serine (SOP), 2-amino-4-(2H-tetrazol-5-yl)butanoic acid, β-glutamic acid (bGlu), 8-aminoquinoline-3-carboxylic acid, biphenylalanine (Bip), 4-benzoylphenylalanine (Bpa), or 3-(9-anthryl)-alanine (H-Ala(9-Anth)-OH or AAP);
X⁴ is absent, asparagine (Asn), glutamine (Gln), aspartic acid (Asp), glutamic acid (Glu), tryptophan (Trp), glycine (Gly), D-alanine (D-Ala), sarcosine (Sar), or arginine (Arg); and
X⁵ is absent, serine (Ser), threonine (Thr), lysine (Lys), asparagine (Asn), glutamine (Gln), aspartic acid (Asp), glutamic acid (Glu), glycine (Gly), alanine (Ala), or sarcosine (Sar).

In some embodiments,
X¹ is absent, D-tyrosine (D-Tyr), glycine (Gly), sarcosine (Sar), D-alanine (D-Ala), aspartic acid (Asp), lysine (Lys), phenylalanine (Phe), or D-3-(3-pyridyl)alanine (3-Pal) (D-3-Pal);
X² is absent, D-asparagine (D-Asn), glutamine (Gln), aspartic acid (Asp), glutamic acid (Glu), D-serine (D-Ser), D-histidine (D-His), alanine (Ala), sarcosine (Sar), tyrosine (Tyr), Proline (Pro), hydroxyproline (Hyp), azetidine-2-carboxylic acid (Aze), 2,3,4,5-tetrahydroisoquinoline-3-carboxylic acid (Tic), phenylalanine (Phe), 3-(2-pyridyl)alanine (2-Pal), 3-(3-pyridyl)alanine (3-Pal), or 3-(4-pyridyl)alanine (4-Pal);
X³ is absent, D-tryptophan (D-Trp), serine (Ser), leucine (Leu), isoleucine (Ile), phenylalanine (Phe), D-3-(2-pyridyl)alanine (D-2-Pal), D-3-(3-pyridyl)alanine (D-3-Pal), D-3-(4-pyridyl)alanine (D-4-Pal), D-lysine (D-Lys), asparagine (Asn), glutamine (Gln), D-aspartic acid (D-Asp), D-glutamic acid (D-Glu), arginine (Arg), methyl arginine (Arg(Me)), norarginine (AGBA), methyl norarginine (AGBA(Me)), homoarginine (HArg), methyl homoarginine (HArg(Me)), citrulline (Cit), methyl citrulline (Cit(Me)), canavanine, methyl-canavanine, glycine (Gly), alanine (Ala), sarcosine (Sar), tyrosine (Tyr), cyclohexylalanine (Cha), 3-(1-naphthyl)alanine(α-Nal), 3-(2-naphthyl)alanine (β-Nal), threonine (Thr), proline (Pro), hydroxyproline (Hyp), tetrahydroisoquinoline-3-carboxylic acid (Tic);
X⁴ is absent, D-asparagine (D-Asn), glutamine (Gln), D-aspartic acid (D-Asp), glutamic acid (Glu), tryptophan (Trp), glycine (Gly), D-alanine (D-Ala), or sarcosine (Sar); and
X⁵ is absent, serine (Ser), threonine (Thr), lysine (Lys), D-asparagine (D-Asn), glutamine (Gln), aspartic acid (Asp), glutamic acid (Glu), glycine (Gly), alanine (Ala), or sarcosine (Sar).

In some embodiments,
X¹ is absent, tyrosine (Tyr), 3-(3-pyridyl)alanine (3-Pal), or γ-glutamic acid (r-Glu);
X² is absent, asparagine (Asn), glutamine (Gln), serine (Ser), histidine (His), or phenylalanine (Phe);
X³ is absent, tryptophan (Trp), isoleucine (Ile), 3-(4-pyridyl)alanine (4-Pal), lysine (Lys), aspartic acid (Asp), glutamic acid (Glu), glycine (Gly), alanine (Ala), cyclohexylalanine (Cha), 3-(2-naphthyl)alanine (β-Nal), hydroxyproline (Hyp), biphenylalanine (Bip), 4-benzoylphenylalanine (Bpa), or 3-(9-anthryl)-alanine (H-Ala(9-Anth)-OH or AAP)
X⁴ is absent, asparagine (Asn), or glutamine (Gln); and
X⁵ is absent, serine (Ser), threonine (Thr), glycine (Gly), or alanine (Ala).

In some embodiments,
X¹ is tyrosine (Tyr), 3-(3-pyridyl)alanine (3-Pal), or γ-glutamic acid (γ-Glu);
X² is absent;
X³ is tryptophan (Trp) isoleucine (Ile), 3-(4-pyridyl)alanine (4-Pal), lysine (Lys), aspartic acid (Asp), glutamic acid (Glu), glycine (Gly), alanine (Ala), cyclohexylalanine (Cha), 3-(2-naphthyl)alanine (β-Nal), hydroxyproline (Hyp), biphenylalanine (Bip), 4-benzoylphenylalanine (Bpa), or 3-(9-anthryl)-alanine (1-Ala(9-Anth)-OH or AAP);
X⁴ is asparagine (Asn) or glutamine (Gln); and
X⁵ is serine (Ser), threonine (Thr), glycine (Gly), or alanine (Ala).

In some embodiments,
X¹ is absent, tyrosine (Tyr), or 3-(3-pyridyl)alanine (3-Pal));
X² is absent, asparagine (Asn), glutamine (Gln), serine (Ser), D-histidine (D-His), or phenylalanine (Phe);
X³ is absent, tryptophan (Trp), isoleucine (Ile), 3-(4-pyridyl)alanine (4-Pal), lysine (Lys), aspartic acid (Asp), glutamic acid (Glu), glycine (Gly), alanine (Ala), cyclohexylalanine (Cha), hydroxyproline (Hyp), biphenylalanine (Bip); 4-benzoylphenylalanine (Bpa), or 3-(9-anthryl)-alanine (AAP);
X⁴ is absent, asparagine (Asn), or glutamine (Gln); and
X⁵ is absent, serine (Ser), threonine (Thr), glycine (Gly), or alanine (Ala).

In some embodiments,
X¹ is tyrosine (Tyr), or 3-(3-pyridyl)alanine (3-Pal));
X² is absent;
X³ is tryptophan (Trp), isoleucine (Ile), 3-(4-pyridyl)alanine (4-Pal), lysine (Lys), aspartic acid (Asp), glutamic acid (Glu), glycine (Gly), alanine (Ala), cyclohexylalanine (Cha), hydroxyproline (Hyp), biphenylalanine (lip); 4-benzoylphenylalanine (Bpa), or 3-(9-anthryl)-alanine (AAP);
X⁴ is asparagine (Asn), or glutamine (Gln); and
X⁵ is serine (Ser), threonine (Thr), glycine (Gly), or alanine (Ala).

In some embodiments, X¹ is not absent; X² is absent; X³ is not absent; X⁴ is not absent; and X⁵ is not absent.

In some embodiments, X¹ is D-tyrosine (D-Tyr); X² is absent; X³ is D-tryptophan (D-Trp), biphenylalanine (Bip); 4-benzoylphenylalanine (Bpa), or 3-(9-anthryl)-alanine (AAP); X⁴ is asparagine (Asn); and X⁵ is serine (Set) or threonine (Thr).

In some embodiments, X¹ is absent, Tyr, Asp, Lys, 3-Pal, Sar, or Phe; and X² is absent, Asn, Gin, Asp, Glu, Ser, His, Ala, Sar, Pro, Hyp, Aze, Tic, Phe, or 4-Pal.

In some embodiments, X¹ is absent, D-Tyr, Asp, Lys, D-3-Pal, Sar, or Phe; and X² is absent, D-Asn, Gin, Asp, Glu, Ser, His, Ala, Sar, Pro, Hyp, Aze, Tic, Phe, or 4-Pal.

In some embodiments, X³ is absent, Trp, Ser, Ile, Phe, 4-Pal, Lys, Asn, Gln, Asp, Glu, Arg, Arg(Me), AGBA, AGBA(Me), Harg, Harg(Me), Cit, Cit(Me), canavanine, methyl-canavanine, Gly, Ala, Sar, Tyr, Cha, β-Nal, Hyp, Thr, Bip, Bpa, or H-Ala(9-Anth)-OH; X⁴ is absent, Asn, Gin, Asp, Glu, Trp, Gly, Ala, or Sar; and X⁵ is absent, Ser, Thr, Asn, Gln, Asp, Glu, Gly, Ala or Sar.

In some embodiments, X³ is absent, Trp, Ser, lie, Phe, 4-Pal, Lys, Asn, Gln, Asp, Glu, Arg, Arg(Me), AGBA, AGBA(Me), Harg, Harg(Me), Cit, Cit(Me) canavanine, methyl-canavanine, Gly, Ala, Sar, Tyr, Cha, β-Nal, Hyp, Thr, Bip, Bpa, or AAP; X⁴ is absent, -Asn, Gin, Asp, Glu, Trp, Gly, Ala, or Sar; and X⁵ is absent, Thr, Ser or Ala.

In some embodiments, X¹ is absent, Tyr, Asp, Lys, 3-Pal, Sar, or Phe; X² is absent, Asn, Gin, Asp, Glu, Ser, His, Ala, Sar, Pro, Hyp, Aze, Tic, Phe, or 4-Pal; X³ is absent, Trp, Ser, lie, Phe, 4-Pal, Lys, Asn, Gin, Asp, Glu, Arg, Arg(Me), Gly, Ala, Sar, Tyr, Cha, β-Nal, Hyp, Thr, Bip, Bpa, or AAP; X⁴ is absent, Asn, Gln, Asp, Glu, Trp, Gly, Ala, or Sar; and X⁵ is absent, Thr, Ser, Gly, or Ala.

In some embodiments, NV is absent, Tyr, Asp, Lys, 3-Pal, Sar, or Phe. In some embodiments, X¹ is absent, D-Tyr, Asp, Lys, D-3-Pal, Sar, or Phe. In some embodiments, X is absent, Tyr, or 3-Pal. In some embodiments, X is absent. In some embodiments, X¹ is Tyr. In some embodiments, X¹ is D-Tyr. In some embodiments, X is 3-Pal.

In some embodiments, X² is absent, Asn, Gln, Asp, Glu, Ser, His, Ala, Sar, Pro, Hyp, Aze, Tic, Phe, or 4-Pal. In some embodiments, V is absent, D-Asn, Gln, Asp, Glu, Ser, His, Ala, Sar, Pro, Hyp, Aze, Tic, Phe, or 4-Pal. In some embodiments, X² is absent, D-Asn, Gln, Asp, Glu, Ala, Sar, Pro, Hyp, Aze, Tic, Phe, or 4-Pal. In some embodiments, X² is absent, Asn, Gin, Ser, or His. In some embodiments, V is absent. In some embodiments, X² is Asn. In some embodiments, X² is Gln. In some embodiments, X² is Ser. In some embodiments, X² is His.

In some embodiments, X³ is absent, Trp, Ser, lie, Phe, 4-Pal, Lys, Asn, Gln, Asp, Glu, Arg, Arg(Me), AGBA, AGBA(Me), HArg, HArg(Me), Cit, Cit(Me), canavanine, methyl-canavanine, Gly, Ala, Sar, Tyr, Cha, β-Nal, Hyp, Thr, Bip, Bpa, or H-Ala(9-Anth)-OH. In some embodiments, X³ is absent, Trp, Ser, Ile, Phe, 4-Pal, Lys, Asn, Gin, Asp, Glu, Arg, Arg(Me), AGBA, AGBA(Me), HArg, HArg(Me), Cit, Cit(Me), canavanine, methyl-canavanine, Gly, Ala, Sar, Tyr, Cha, β-Nal, Hyp, or Thr. In some embodiments, X³ is X³ is absent, D-Trp, Ser, Ile, Phe, 4-Pal, D-Lys, Asn, Gin, D-Asp, D-Glu, Arg, Arg(Me), AGBA, AGBA(Me), HArg, HArg(Me), Cit, Cit(Me), canavanine, methyl-canavanine, Gly, Ala, Sat, Tyr, Cha, β-Nal, Hyp, or Thr. In some embodiments, X³ is absent, D-Trp, Ser, Ile, Phe, 4-Pal, D-Lys, Asn, Gln, D-Asp, D-Glu, Arg, Arg(Me), Gly, Ala, Sar, Tyr, Cha, β-Nal, Hyp, or Thr. In some embodiments, X³ is absent, Trp, Ile, 4-Pal, Lys, Asp, Glu, Gly, Ala, Cha, β-Nal, Hyp, Bip, Bpa, or AAP. In some embodiments, X³ is absent, Ile, 4-Pal, Lys, Asp, Glu, Gly, Ala, Cha, β-Nal, or Hyp. In some embodiments, X³ is Trp, Bip, Bpa, or AAP. In some embodiments, X³ is absent. In some embodiments, X³ is Trp. In some embodiments, X³ is Ile. In some embodiments, X³ is 4-Pal. In some embodiments, X³ is Lys. In some embodiments, X³ is Asp. In some embodiments, X³ is Glu. In some embodiments, X³ is Gly. In some embodiments, X³ is Ala. In some embodiments, X³ is Cha. In some embodiments, X³ is β-Nal. In some embodiments, X³ is Hyp. In some embodiments, X³ is Bip. In some embodiments, X³ is Bpa. In some embodiments, X³ is AAP

In some embodiments, X⁴ is absent, Asn, Gin, Asp, Glu, Trp, Gly, Ala, or Sar. In some embodiments, X⁴ is D-Asn, Gin, Asp, Glu, Trp, Gly, Ala, or Sar. In some embodiments, X⁴ is absent, Asn, or Gin. In some embodiments, X⁴ is absent. In some embodiments, X⁴ is Asn. In some embodiments, X⁴ is Gin.

In some embodiments, X⁵ is absent, Ser, Thr, Asn, Gln, Asp, Glu, Gly, Ala or Sar. In some embodiments, X⁵ is absent, Thr, Ser or Ala. In some embodiments, X⁵ is absent, Ser, Thr, Gly, or Ala. In some embodiments, X⁵ is absent. In some embodiments, X⁵ is Ser. In some embodiments, X⁵ is Thr. In some embodiments, X⁵ is Gly. In some embodiments, X⁵ is Ala.

In some embodiments, X⁶ is Phe, 3-F-Phe, Bip, ββ-(2-thienyl)-Ala), Cha, or Tyr. In some embodiments, X⁶ is Phe. In some embodiments, X⁶ is 3-F-Phe. In some embodiments, X⁶ is Bip. In some embodiments, X⁶ is β-(2-thienyl)-Ala. In some embodiments, X⁶ is Cha. In some embodiments, X⁶ is Tyr.

In some embodiments, X⁷ is Gly or azaGly. In some embodiments, X⁷ is Gly. In some embodiments, X⁷ is azaGly.

In some embodiments, X⁸ is Leu or Nva. In some embodiments, X⁸ is Leu. In some embodiments, X⁸ is Nva.

In some embodiments, X⁸ is Leu, Nva, Ile, HAla, or Phe, and X¹⁰ is Trp, 1MT, Tyr, 4-Pal, Phe(4-CN) or Phe.

In some embodiments, X⁶ is

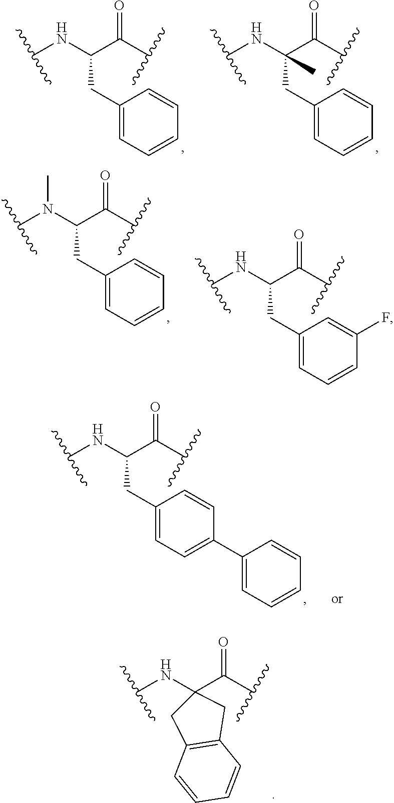

In some embodiments X⁶ is

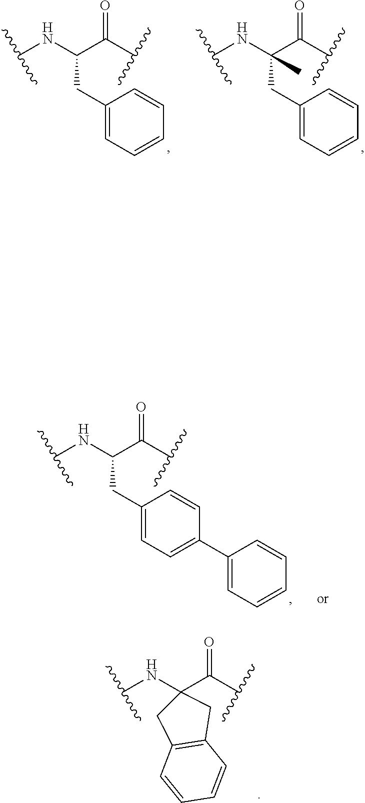

In some embodiments, X⁷ is

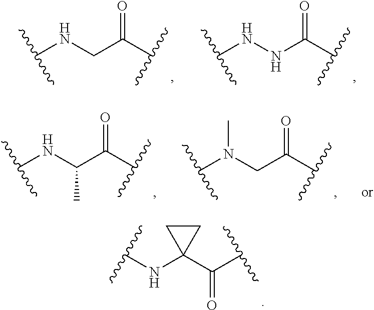

In some embodiments, X⁷ is

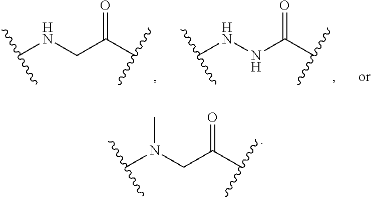

In some embodiments, X⁸ is

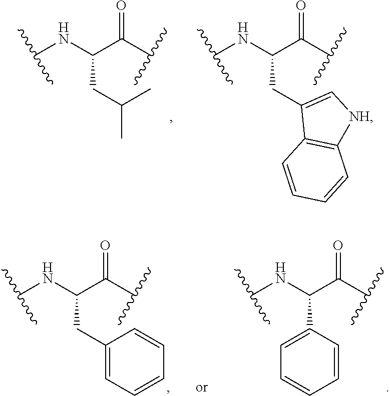

In some embodiments, X⁸ is

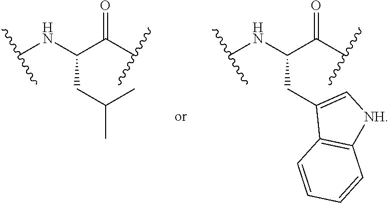

In some embodiments,
X⁶ is

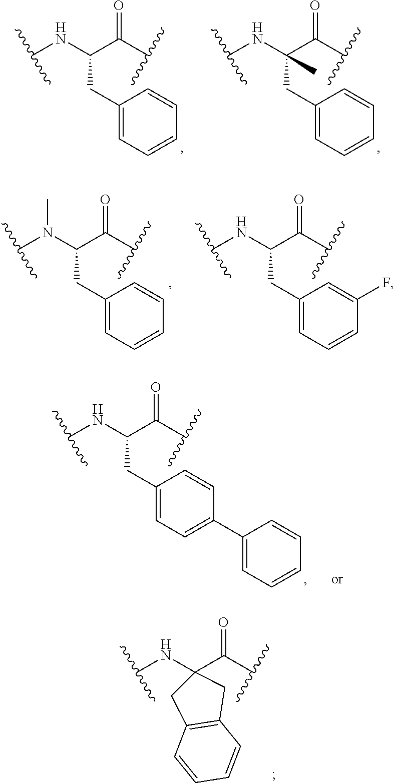

X⁷ is

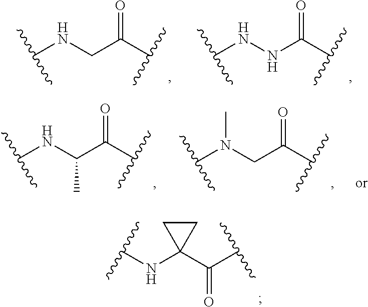

X⁸ is

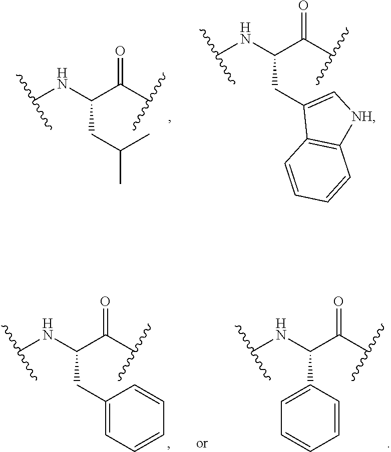

In some embodiments,
X⁶ is

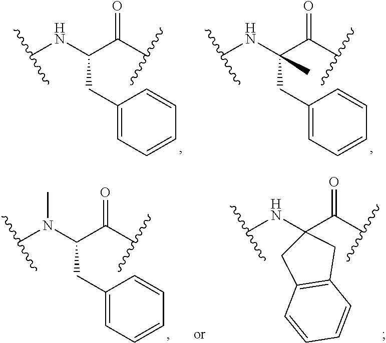

X⁷—X⁸ is

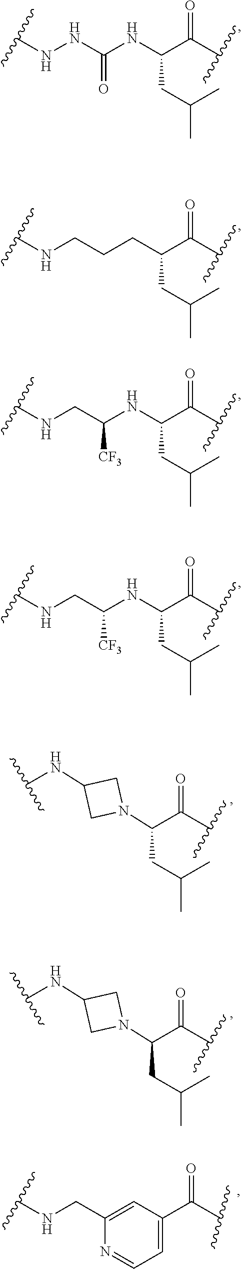

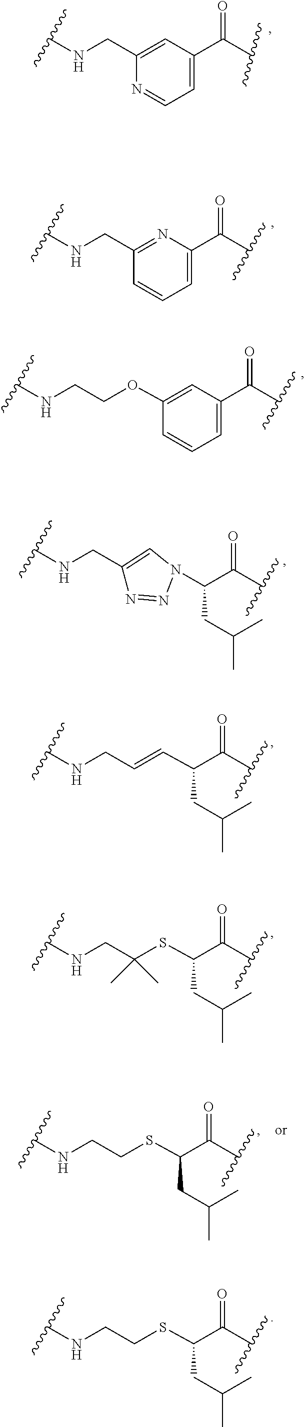

In some embodiments, —X⁶—X⁷—X⁸— is

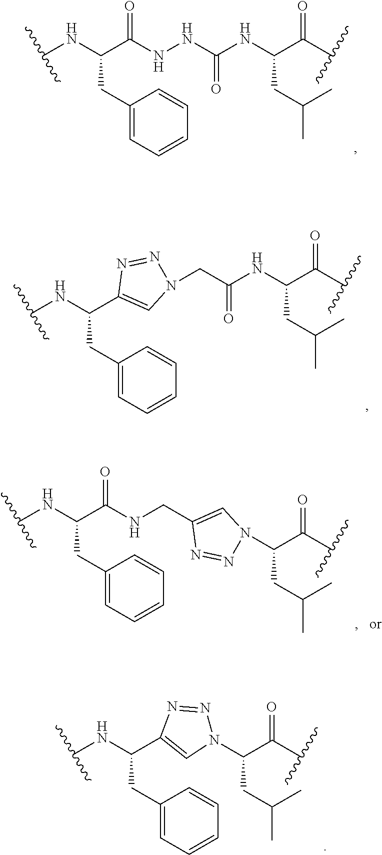 [SMILES: *N[C@@H](Cc1ccccc1)C(=O)NNC(=O)N[C@@H](CC(C)C)C(*)=O ; *N[C@@H](Cc1ccccc1)c1cn(CC(=O)N[C@@H](CC(C)C)C(*)=O)nn1 ; *N[C@@H](Cc1ccccc1)C(=O)NCc1cn([C@@H](CC(C)C)C(*)=O)nn1 ; *N[C@@H](Cc1ccccc1)c1cn([C@@H](CC(C)C)C(*)=O)nn1]

In any embodiment described herein,

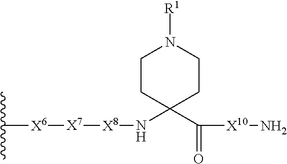

is

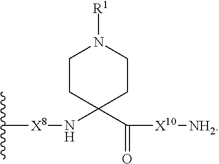

In some embodiments, X⁶ is absent, X⁷ is absent, X⁸ is not absent and X¹⁰ is not absent.

In any embodiment described herein,

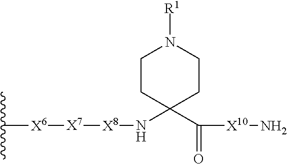

is

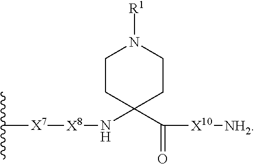

In some embodiments. X⁶ is absent, X⁷ is not absent, X⁸ is not absent, and X¹⁰ is not absent. In some embodiments, X⁶ is absent, X⁷ is not absent, X⁸ is not absent, and X¹⁰ is Trp or Tyr. In some embodiments, X⁶ is absent, X⁷ is AzaGly, X⁸ is not absent, and X¹⁰ is Trp or Tyr. In some embodiments, X⁶ is absent, X⁷ is AzaGly, X⁸ is Leu, and X¹⁰ is Trp or Tyr.

In some embodiments, X⁶ is not absent, X⁷ is not absent, X⁸ is not absent, and X¹⁰ is Trp or Tyr. In some embodiments, X⁶ is AzaGly, X⁷ is not absent, X⁸ is not absent, and X¹⁰ is Trp or Tyr. In some embodiments, X⁶ is AzaGly, X⁷ is Leu, X⁸ is not absent, and X¹⁰ is Trp or Tyr.

In some embodiments, X¹⁰ is Trp, 1MT, Tyr, Phe, Phe(4-CN), 4-Pal, Leu, Phg, Cha, α-Nal, or β-Nal. In some embodiments, X¹⁰ is Trp, 1MT, Tyr, Phe, Phe(4-CN), 4-Pal, or Leu. In some embodiments, X¹⁰ is Trp, Tyr, Phe, or Phe(4-CN). In some embodiments, X¹⁰ is 1MT, 4-Pal, or Leu. In some embodiments, X¹⁰ is Trp. In some embodiments, X¹⁰ is Tyr. In some embodiments, X¹⁰ is Phe. In some embodiments, X¹⁰ is Phe(4-CN). In some embodiments, X¹⁰ is 1MT. In some embodiments, X¹⁰ is 4-Pal. In some embodiments, X¹⁰ is Leu.

In some embodiments, X¹ is absent; X² is absent; X³ is absent; X⁴ is absent; and X⁵ is absent; X⁶ is absent, X⁷ is absent, X⁸ is not absent, and X¹⁰ is not absent.

In some embodiments, X¹ is absent; X² is absent; X¹ is absent; X⁴ is absent; and X⁵ is absent: X⁶ is absent, X⁷ is not absent, X⁸ is not absent, and X¹⁰ is not absent.

In some embodiments, X¹ is absent; X² is absent; X³ is absent; X⁴ is absent; and X⁵ is absent: X⁶ is not absent, X⁷ is not absent, X⁸ is not absent, and X¹⁰ is not absent.

In some embodiments, X¹ is absent; X² is absent; X³ is absent; X⁴ is absent; and X⁵ is not absent; X⁶ is not absent, X⁷ is not absent, X⁸ is not absent, and X¹⁰ is not absent.

In some embodiments, X¹ is absent; X² is absent; X³ is absent; X⁴ is not absent; and X⁵ is not absent; X⁶ is not absent, X⁷ is not absent, X⁸ is not absent, and X¹⁰ is not absent.

In some embodiments, X¹ is absent; X² is absent; X³ is not absent; X⁴ is not absent; and X⁵ is not absent; X⁶ is not absent, X⁷ is not absent, X⁸ is not absent, and X¹⁰ is not absent.

In some embodiments, NV is not absent: X¹ is absent: X³ is not absent; X⁴ is not absent; and X⁵ is not absent; X⁶ is not absent, X⁷ is not absent, X⁸ is not absent, and X¹¹ is not absent.

In some embodiments, X¹ is not absent; X¹ is absent; X³ is not absent; NV is not absent; and X⁵ is not absent: X⁶ is not absent, X⁷ is not absent, X⁸ is not absent, and X¹⁰ is not absent. In some embodiments, X¹ is absent, Tyr, Asp, Lys, 3-Pal, Sar, or Phe; NV is absent; X³ is absent, Trp, Ile, 4-Pal, Lys, Asp, Glu, Gly, Ala, Cha, β-Nal, Hyp, Bip, Bpa, or AAP; X⁴ is Asn, or Gln; X⁵ is Ser, Thr, Gly, or Ala; X⁶ is Phe, 3-F-Phe, Bip, β-(2-thienyl)-Ala), Cha, or Tyr; X⁷ is Gly or azaGly; X⁸ is Leu or Nva; X⁸ is Leu, Nva, Ile, HAla, or Phe, and X¹⁰ is Trp, 1MT, Tyr, 4-Pal, Phe(4-CN) or Phe.

In some embodiments, the N-terminal amino acid or the compound of Formula (I) is optionally substituted with —C(═O)—C₁-C₂₀alkyl, —C(═O)—(CH₂CH₂O)_{y}—CH₂CH₂—R¹⁵, C₁-C₂₀ alkyl N-hexadecanoyl-Glu, C₄-C₂₀ polyethylene glycol, a saccharide, —R¹⁶, —C(═O)—(CH₂CH₂O)ₓ—CH₃, —C(═O)—(CH₂CH₂O)ₓ—H, —C(═O)—CH₂CH₂CH(COOH)—R¹⁵, —C(═O)—(CH₂)₂R¹⁹, or —C(═O)CH₂NHCH₂R¹⁹; R¹⁵ is selected from —OR¹⁶, —N(R¹⁶)₂, —CO(═O)OR¹⁶, or —C(═O)N(R¹⁶)₂; each R¹⁶ is independently H, —C₁-C₆ alkyl, —C(═O)—(CH₂)ᵥR¹⁹, —C(═O)CH₂NHCH₂R¹⁹, or a saccharide or derivative thereof; R¹⁹ is 4-iodophenylene, 4-methylphenylene, or 3-fluoro-4-methylphenylene; y is 0, 1, 2, 3, 4, 5, 6, 7, 8, 9, or 10; x is an integer from 1 and 25; and v is 1, 2, 3, or 4.

In some embodiments, the N-terminal amino acid or the compound of Formula (I) is optionally substituted with —C(═O)—C₁-C₂₀ alkyl. In some embodiments, the N-terminal amino acid or the compound of Formula (I) is optionally substituted with —C(═O)—(CH₂)₂R¹⁹. In some embodiments, the N-terminal amino acid or the compound of Formula (I) is substituted with —R¹⁶. In some embodiments, the N-terminal amino acid or the compound of Formula (I) is substituted with —R¹⁶ and R¹⁶ is C(═O)—(CH₂)ᵥR¹⁹. In some embodiments, the N-terminal amino acid or the compound of Formula (I) is substituted with —C(═O)—(CH₂CH₂O)_{y}—CH₂CH₂—R¹⁵. In some embodiments, the N-terminal amino acid or the compound of Formula (I) is substituted with —C(═O)—(CH₂CH₂O)_{y}—CH₂CH₂—R¹⁵, R¹⁵ is —OR¹⁶ or —N(R¹⁶)₂, and each R¹⁶ is independently H, —C₁-C₆ alkyl, or —C(═O)—(CH₂)ᵥR¹⁹. In some embodiments, y is 2. In some embodiments, v is 2. In some embodiments, R¹⁶ is H or —CH₃. In some embodiments, R¹⁹ is 4-iodophenylene or 4-methylphenylene.

In some embodiments, R¹⁵ is

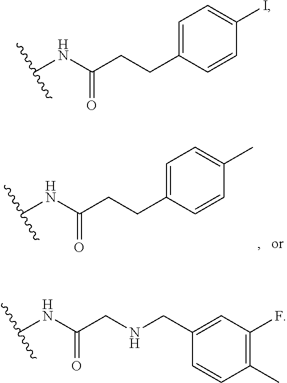 [SMILES: *NC(=O)CCc1ccc(I)cc1 ; *NC(=O)CCc1ccc(C)cc1 ; *NC(=O)CNCc1ccc(C)c(F)c1]

In some embodiments, the N-terminal amino acid or the compound of Formula (I) is substituted with —C(═O)—(CH₂CH₂O)_{y}—CH₂CH₂—R¹⁵ and R¹⁵ is

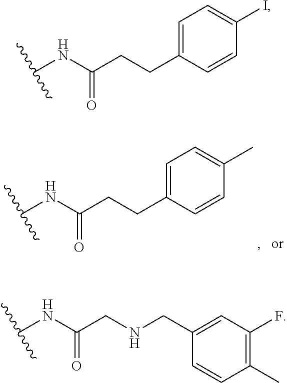 [SMILES: *NC(=O)CCc1ccc(I)cc1 ; *NC(=O)CCc1ccc(C)cc1 ; *NC(=O)CNCc1ccc(C)c(F)c1]

In some embodiments, x is 3 or 9 or 25.

In some embodiments, the N-terminal amino acid or the compound of Formula (I) is optionally substituted with

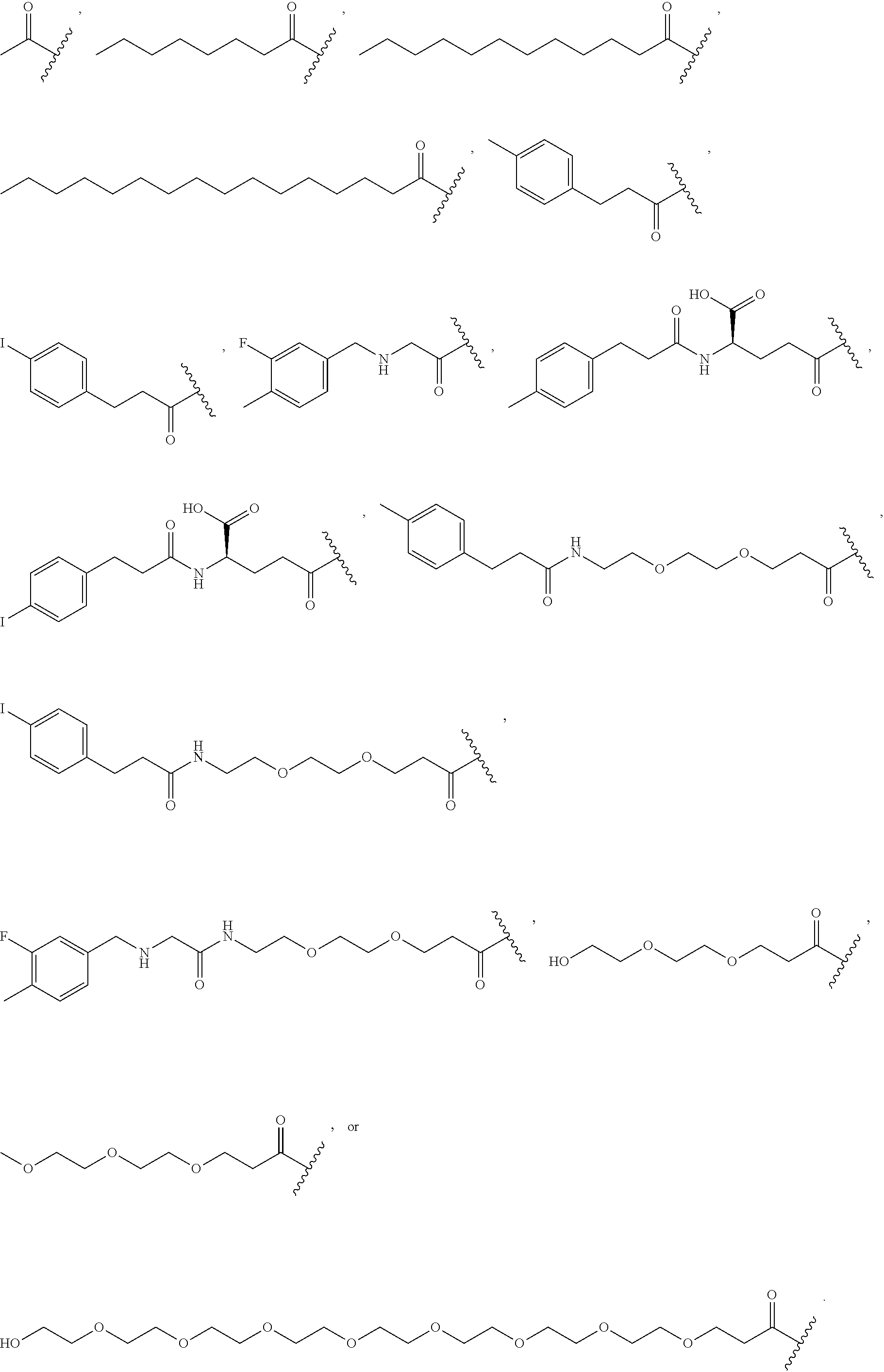

In some embodiments, R¹ is

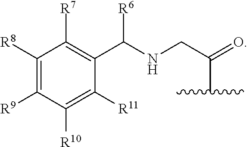

In some embodiments, R¹ is

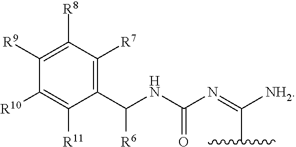

In some embodiments, R¹ is

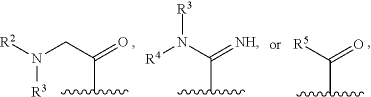

In some embodiments, R¹ is H.

In some embodiments, R¹ is

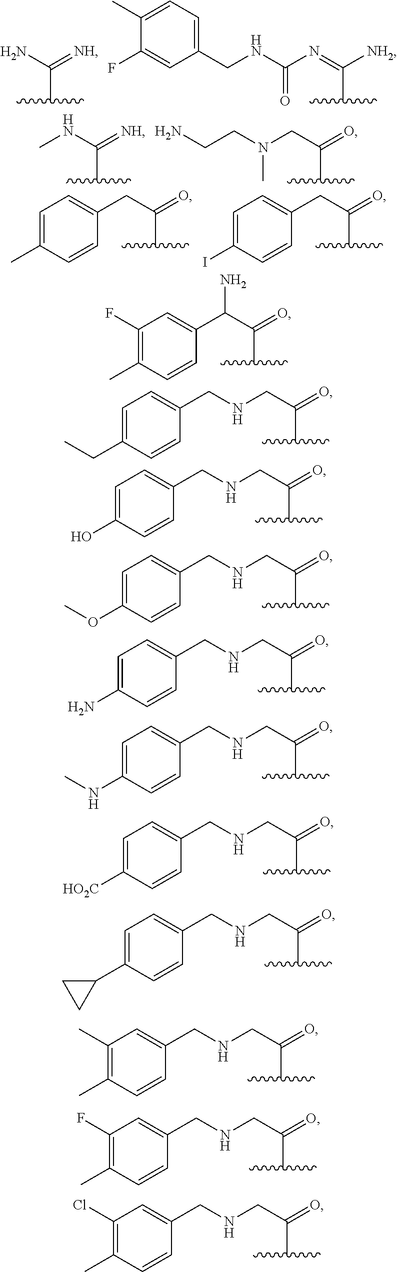

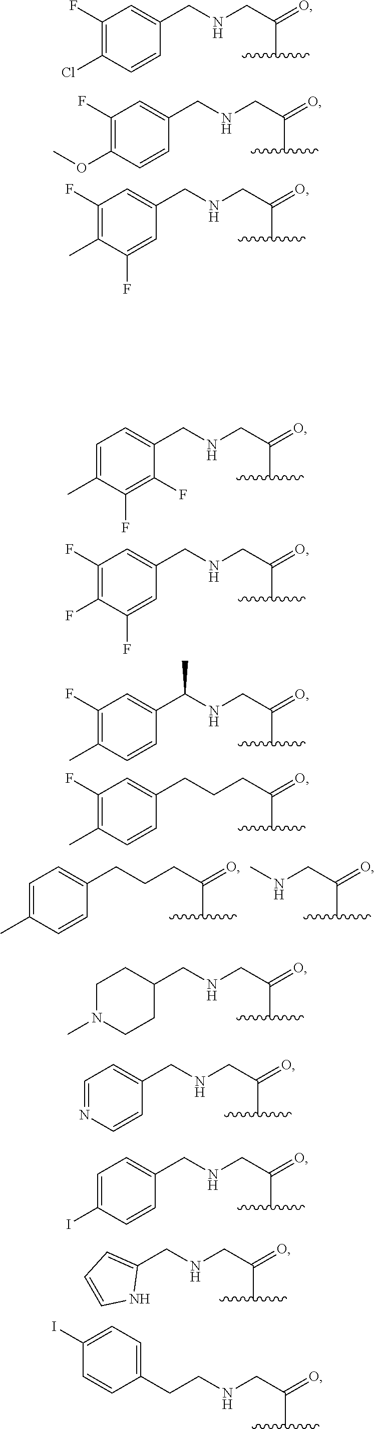

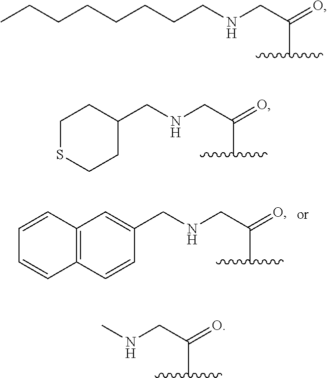

In some embodiments, R¹ is

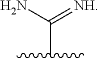 [SMILES: *C(=N)N]

In some embodiments, R¹ is

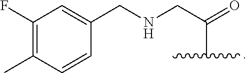 [SMILES: *C(=O)CNCc1ccc(C)c(F)c1]

some embodiments, R¹

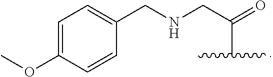 [SMILES: *C(=O)CNCc1ccc(OC)cc1]

In some embodiments, R¹ is

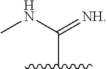 [SMILES: *C(=N)NC]

In some embodiments, R¹ is

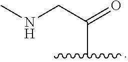 [SMILES: *C(=O)CNC]

In some embodiments, R² is

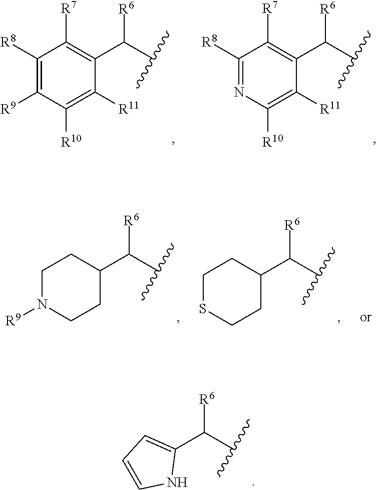

In some embodiments, R² is

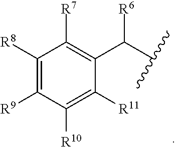

In some embodiments, R² is C₁-C₆ alkyl, wherein C₁-C₆ alkyl is optionally substituted with R⁷.

In some embodiments, R² is

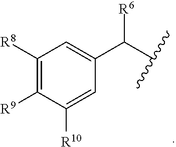

In some embodiments, R³ is H or —CH₃, and R⁴ is H, —CH₃, or R².

In some embodiments, R⁵ is

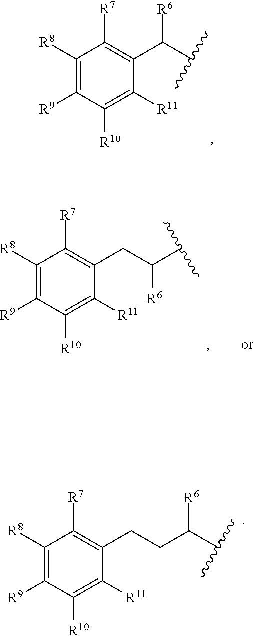

In some embodiments, R⁷, R⁸, R⁹, R¹⁰, and R¹¹ are each independently selected from H, F, Cl, Br, I, —OH, —OCH₃, —OCH₂CH₃, —NH₂, —NHCH₃, —N(CH₃)₂, —CN, —CO₂H, —CO₂CH₃, —CO₂CH₂CH₃, —CH₃, —CH₂CH₃, —CH(CH₃)₂—(CH₃)₃, —CF₃, —CH₂F, —CH₂F, or cyclopropyl.

In some embodiments, R⁷ is H. In some embodiments, R⁸ is F, Cl, Br, I, —CH₃, —CH₂CH₃, or —CF₃. In some embodiments, R⁷ is H, F, Cl, Br, I, —OH, —OCH₃, —OCH₂CH₃, —NH₂, —NHCH₃, —N(CH₃)₂, —CO₂H, —CO₂CH₃, —CO₂CH₂CH₃, —CH₃, —CH₂CH₃, —CH(CH₃)₂, —(CH₃)₃, —CF₃, —CH₂F, —CH₂F, or cyclopropyl. In some embodiments, R¹⁰ is F, Cl, Br, I, —CH₃, —CH₂CH₃, or —CF₃. In some embodiments, R¹¹ is H, F, Cl, Br, or I.

In some embodiments, R⁸ is F, Cl, Br, or I and R⁹ is —CH₃, —CH₂CH₃, or —CF₃.

In some embodiments, R⁸ is H or F and R⁹ is —CH₃, —OCH₃.

In some embodiments,

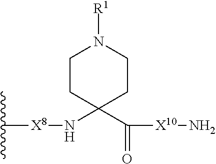

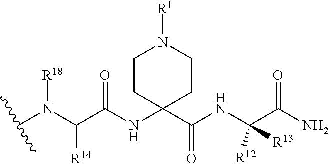

wherein, R¹⁸ is H or —CH₃;
R¹² is

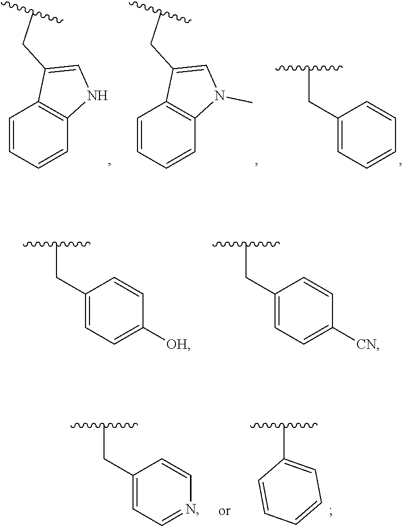

or
R¹³ is H or —CH₃; and
R¹⁴ is

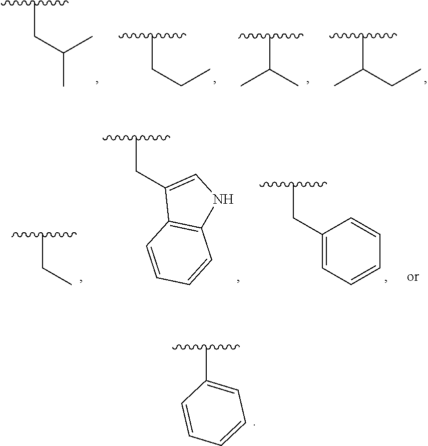

In some embodiments, R¹⁸ is H or —CH₃, and R¹² is

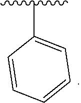 [SMILES: *c1ccccc1]

In some embodiments, R¹² is

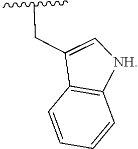 [SMILES: *Cc1c[nH]c2ccccc12]

In some embodiments, R¹⁴ is

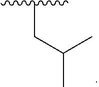 [SMILES: *CC(C)C]

In some embodiments,

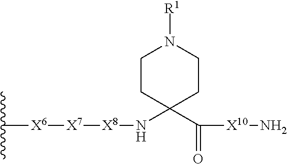

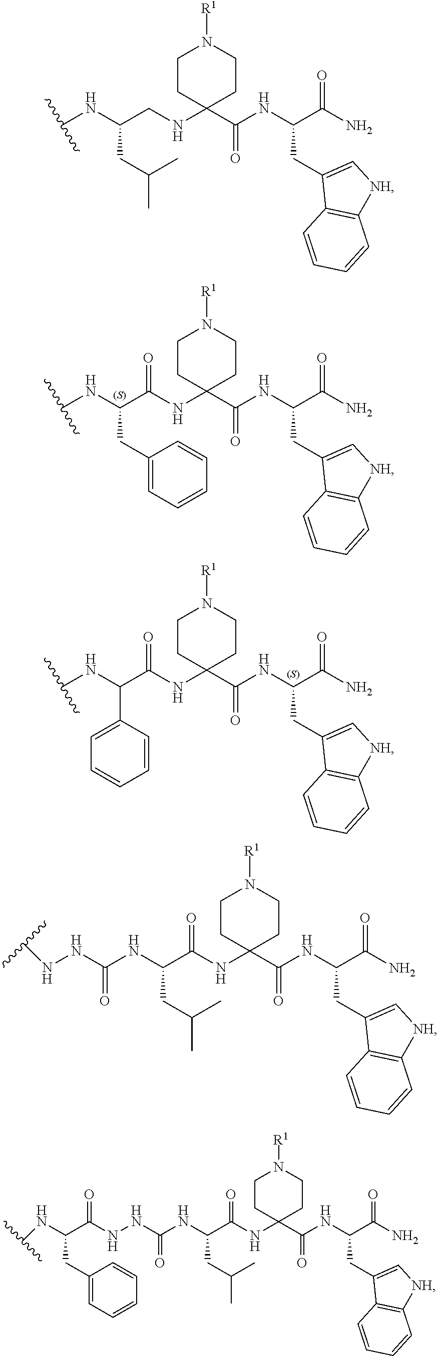

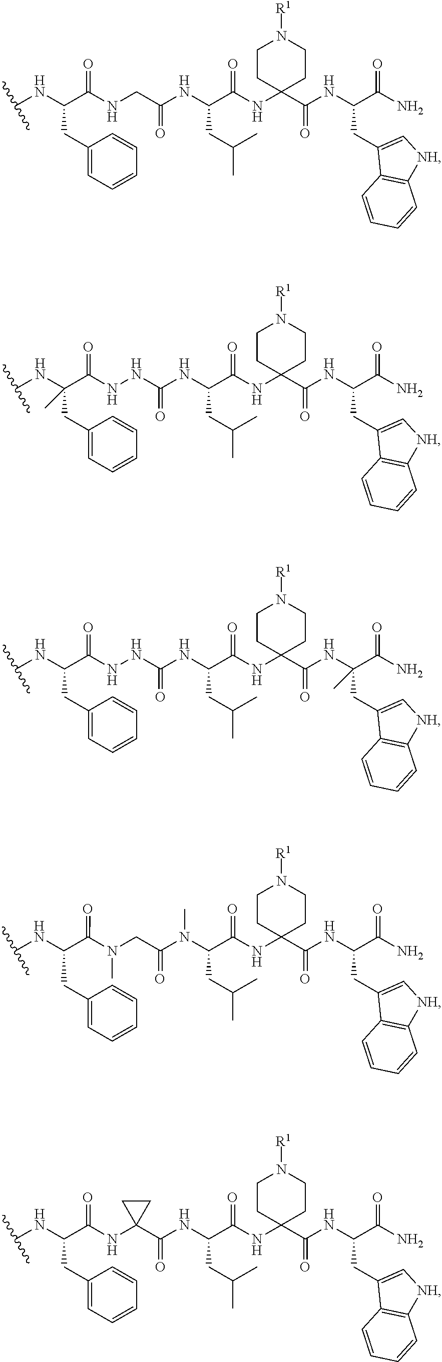

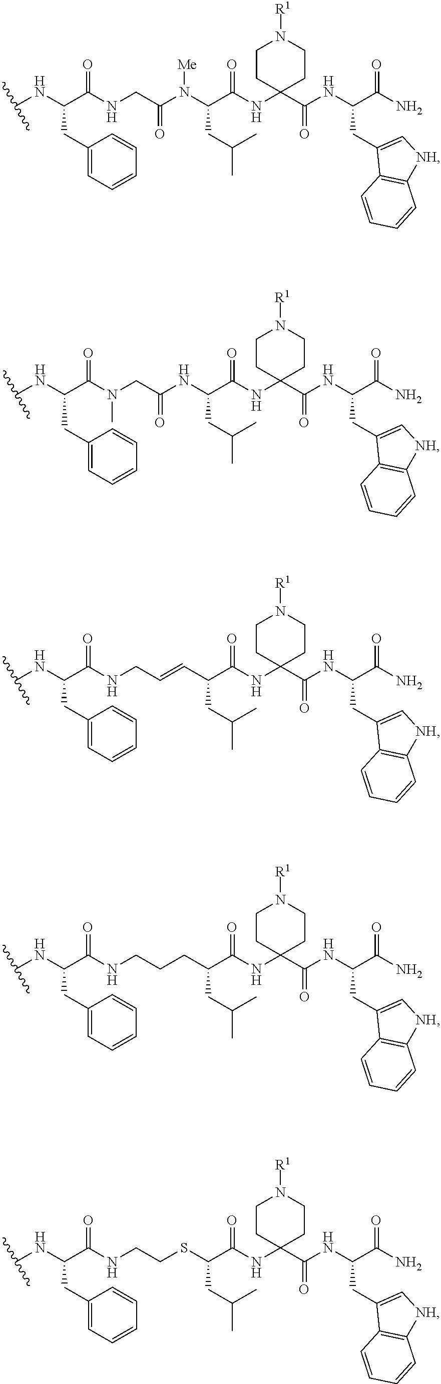

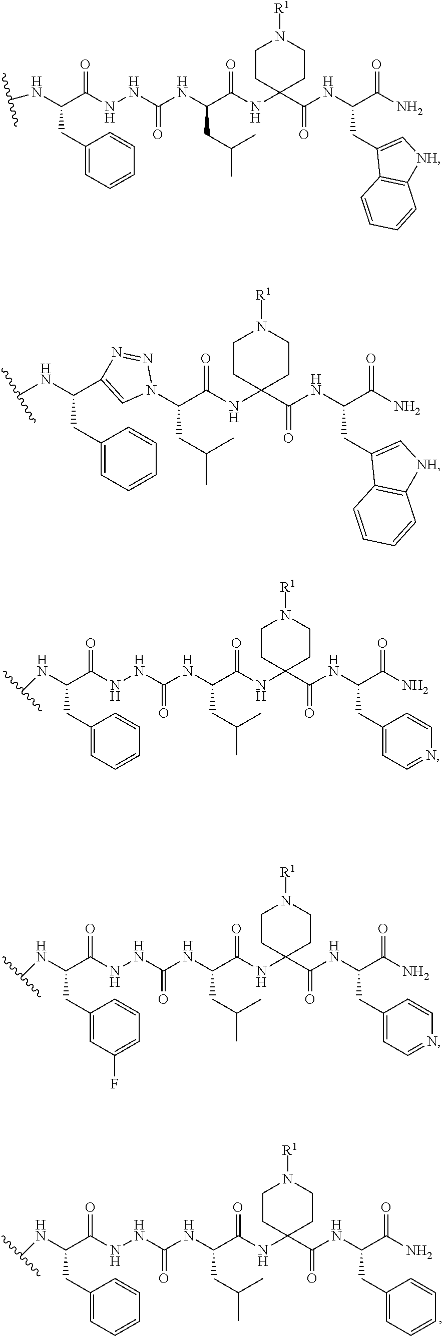

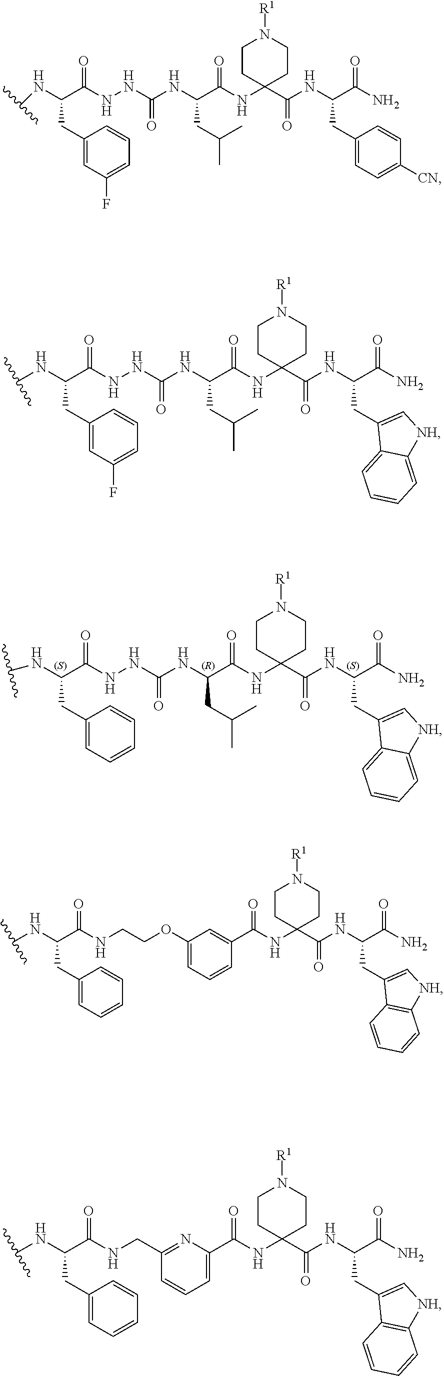

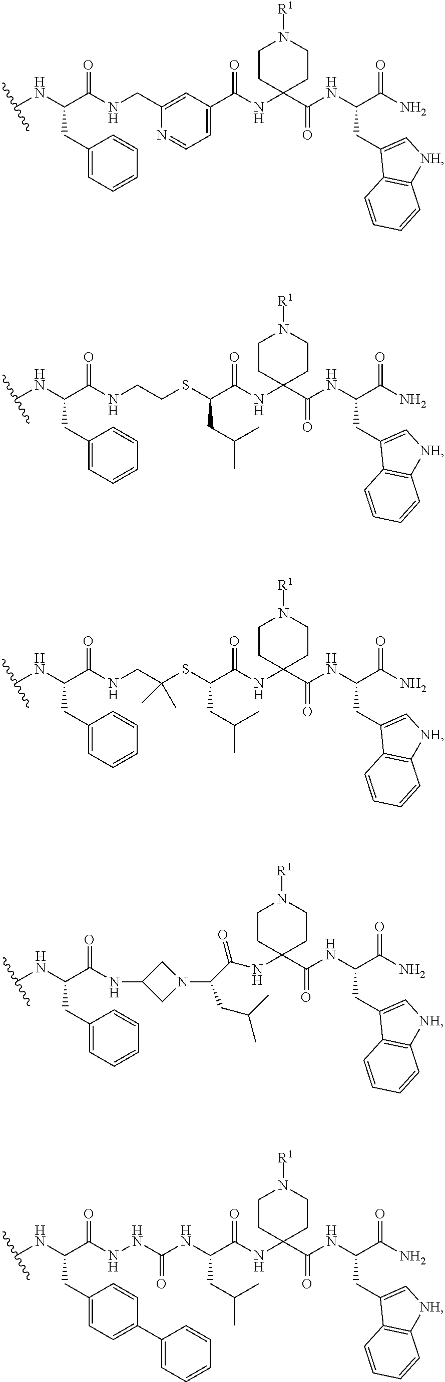

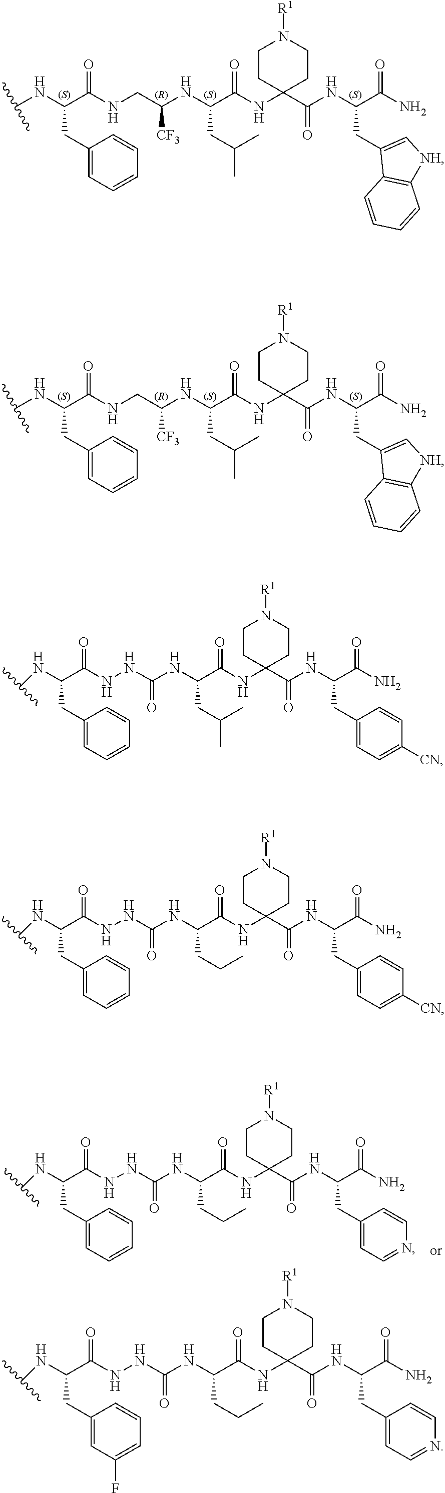

In some embodiments,

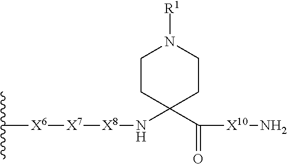

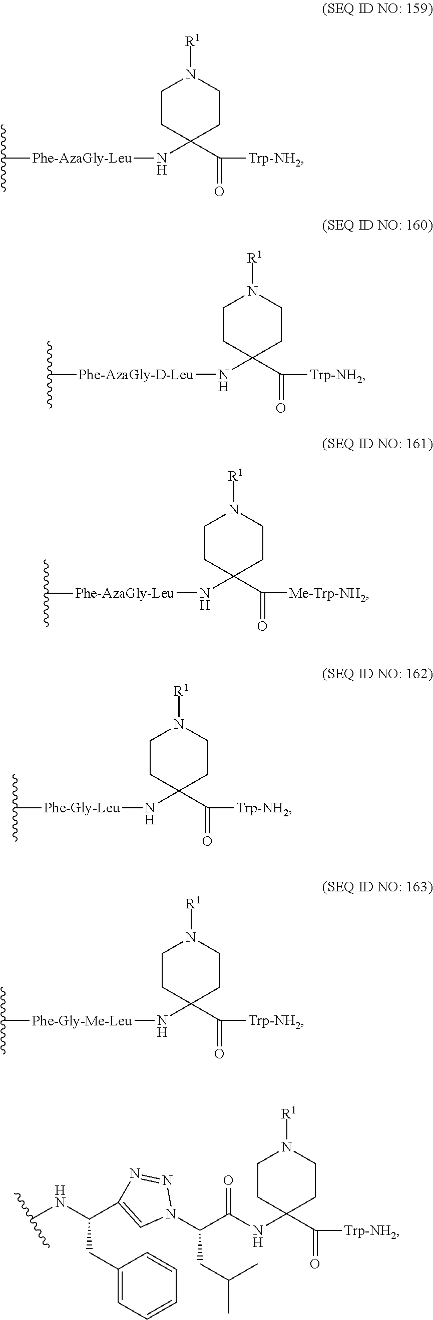

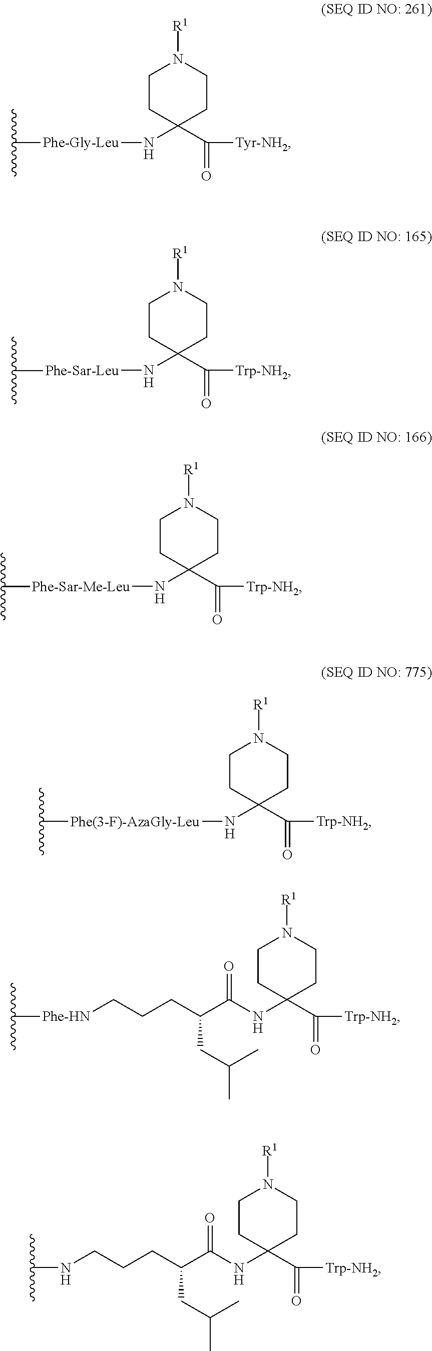

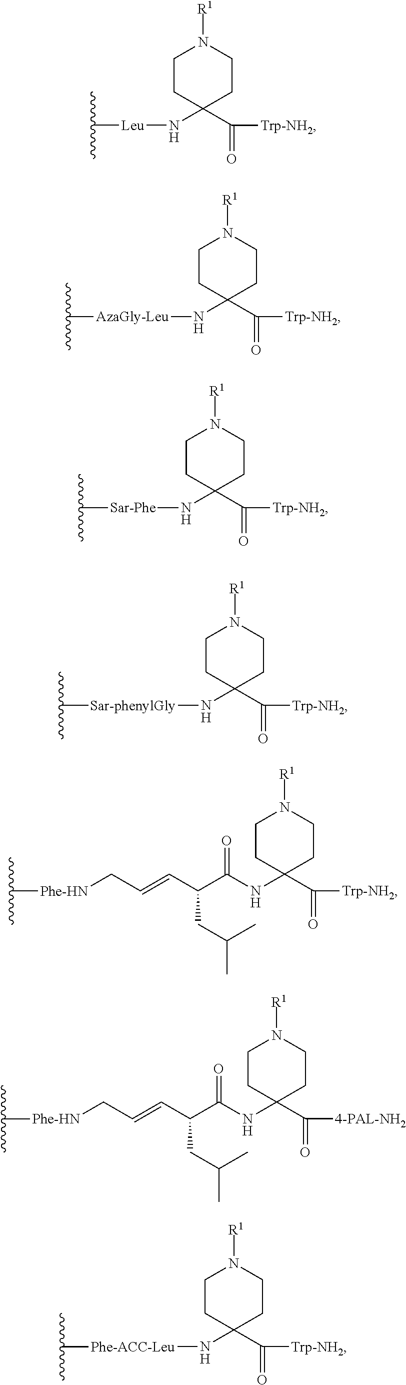

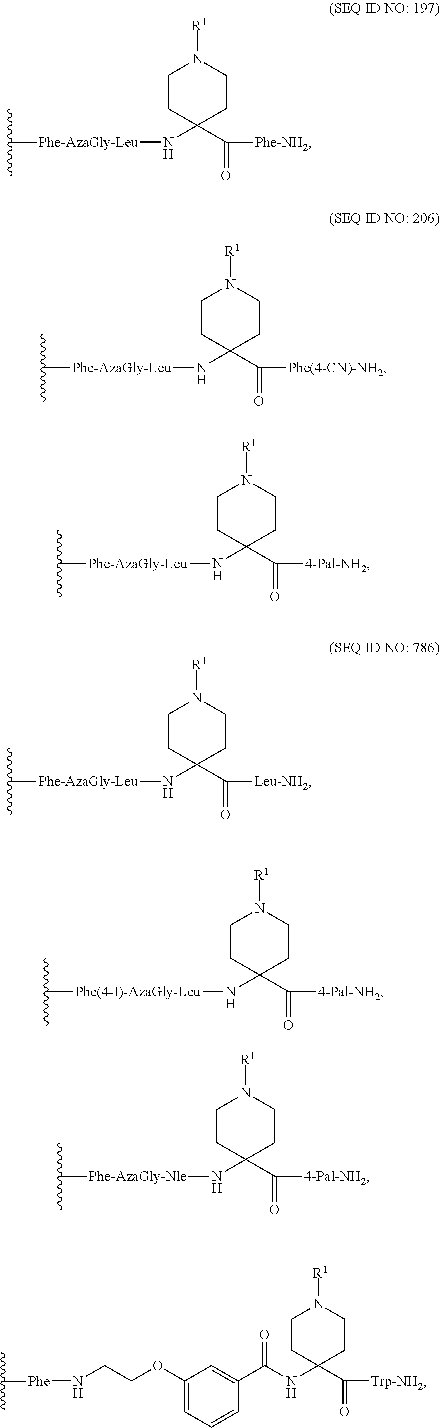

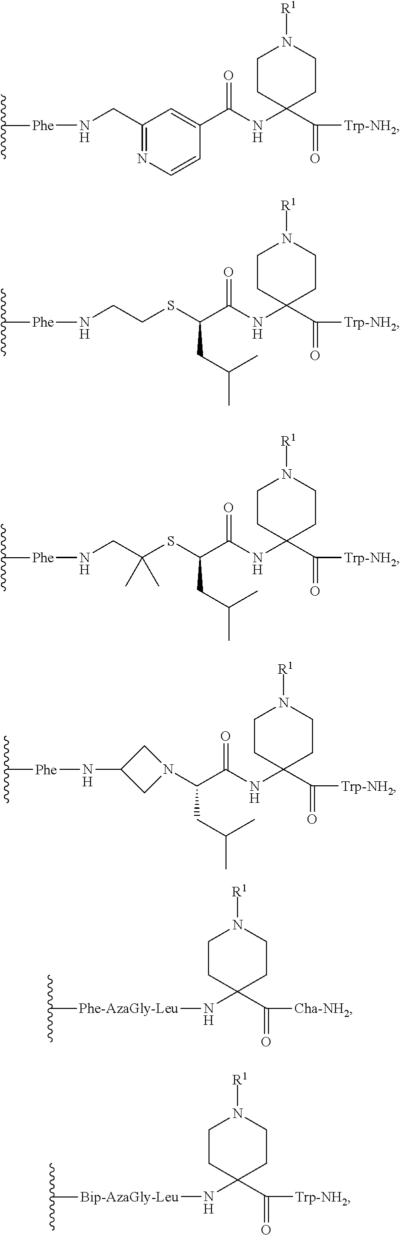

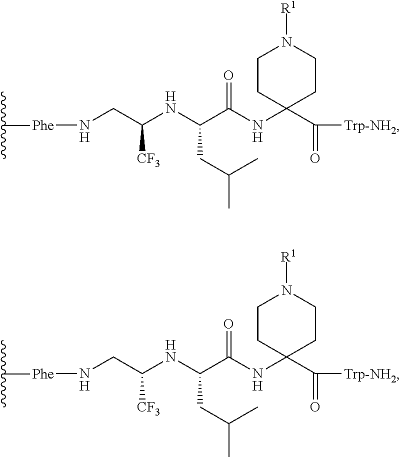

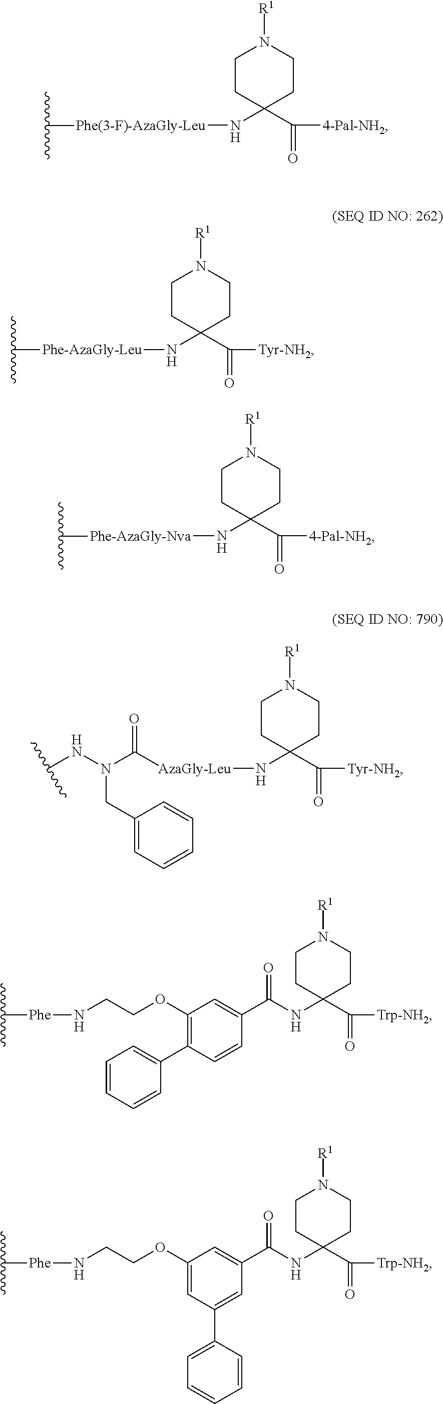

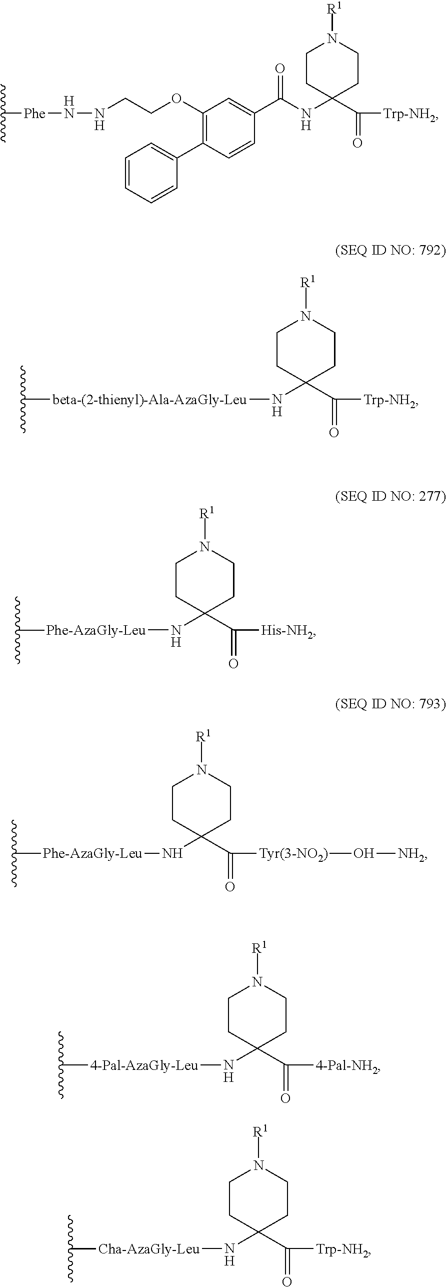

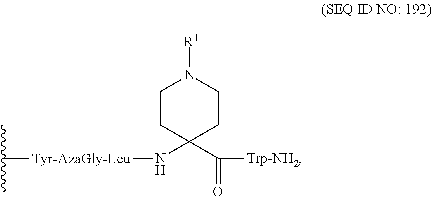

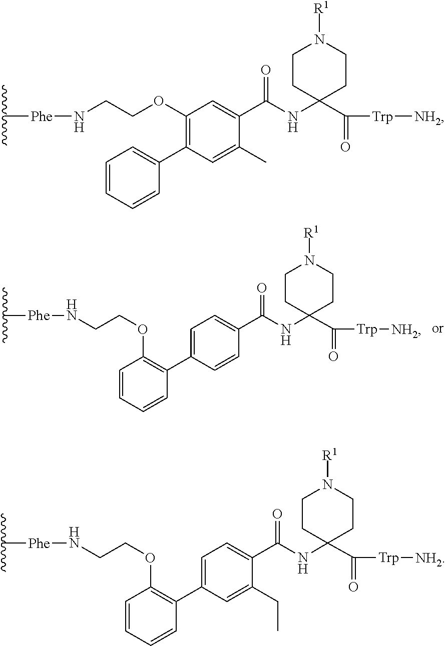

In some embodiments,

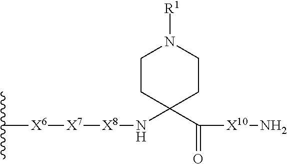

is

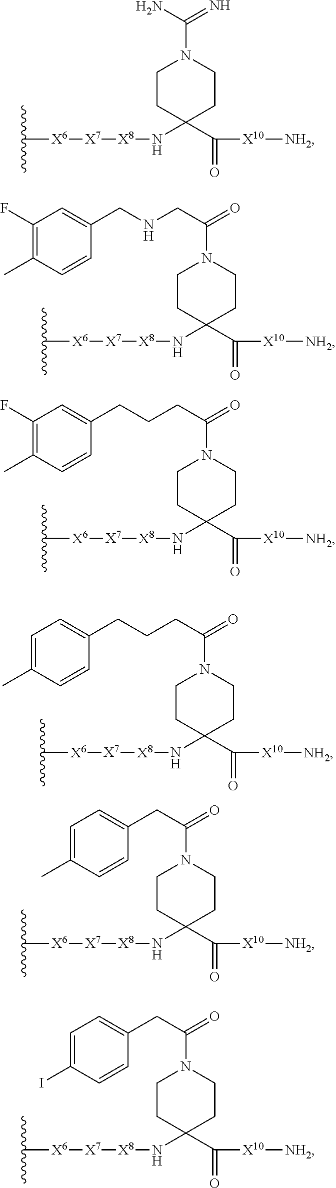

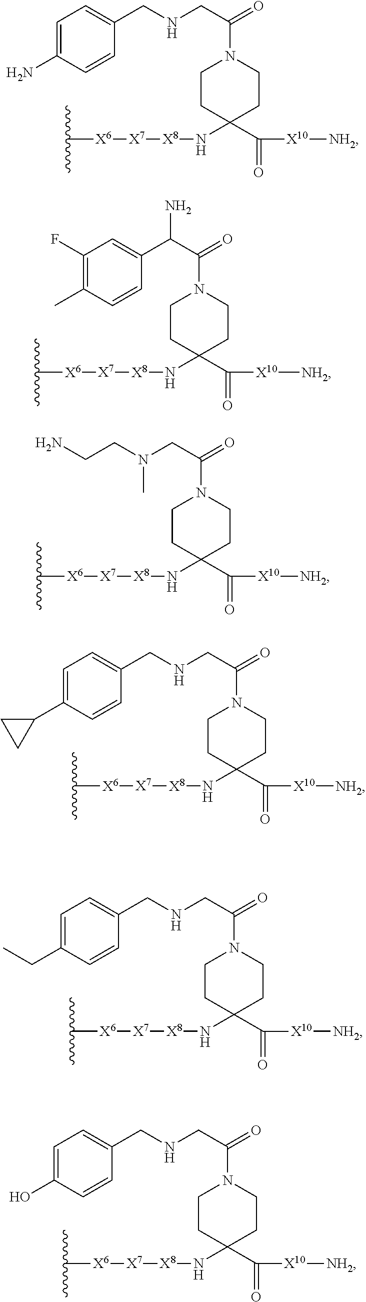

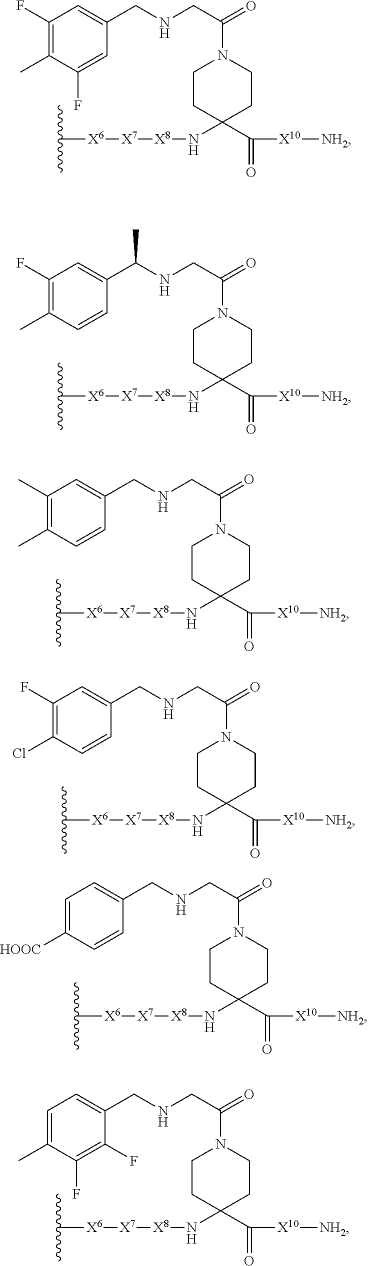

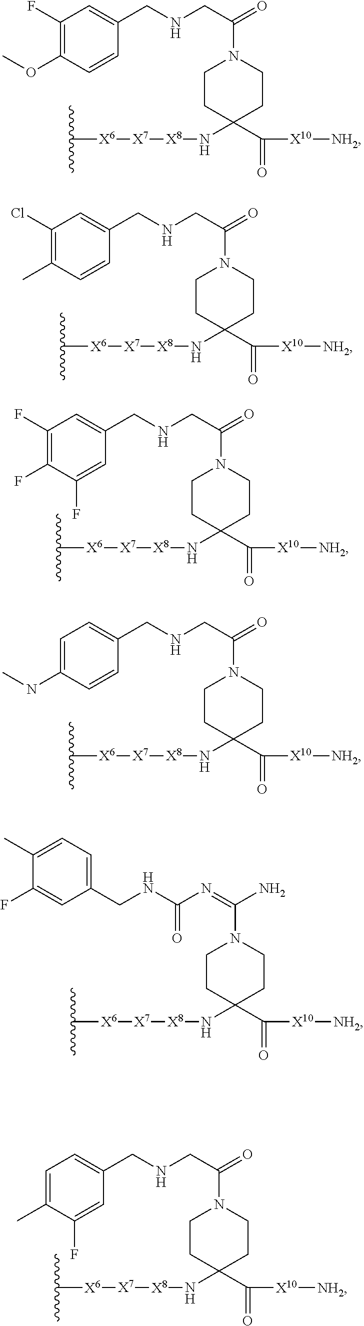

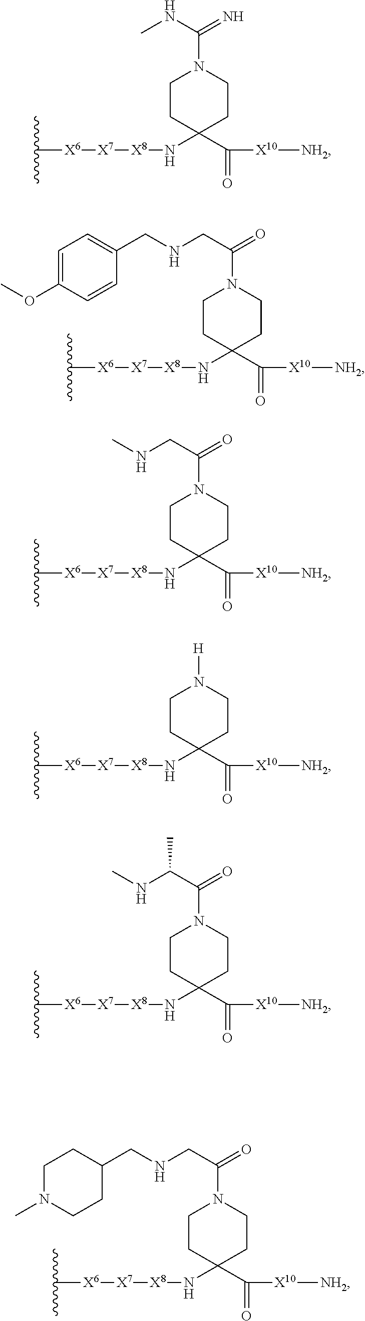

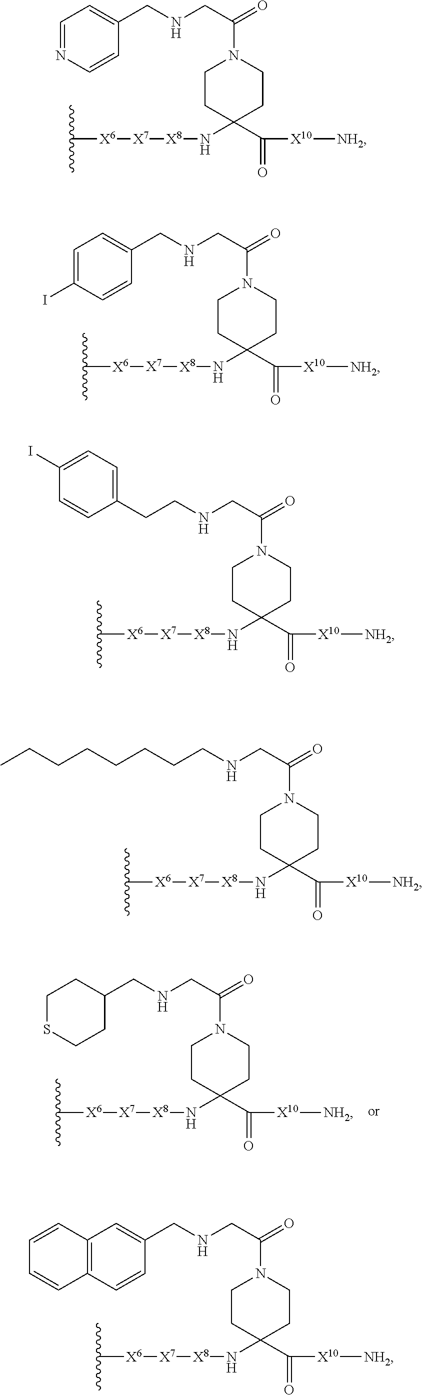

In some embodiments,

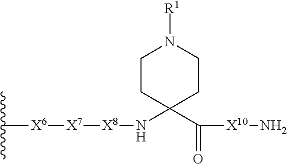

is

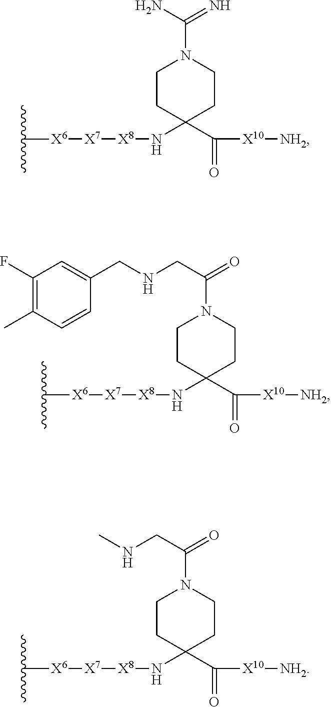

In some embodiments

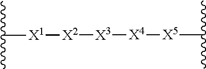

is absent

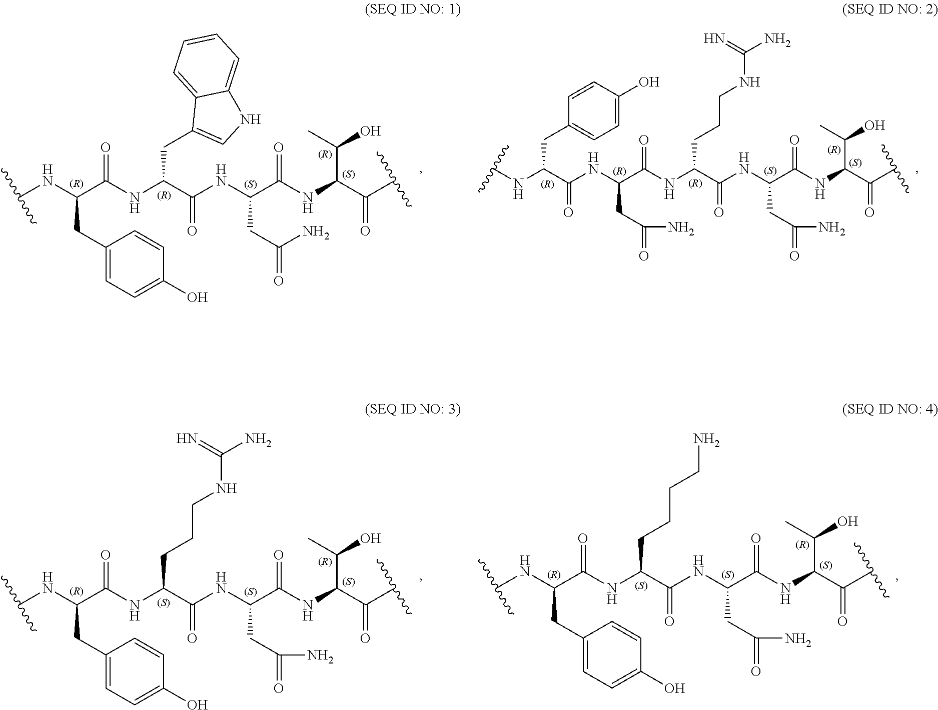

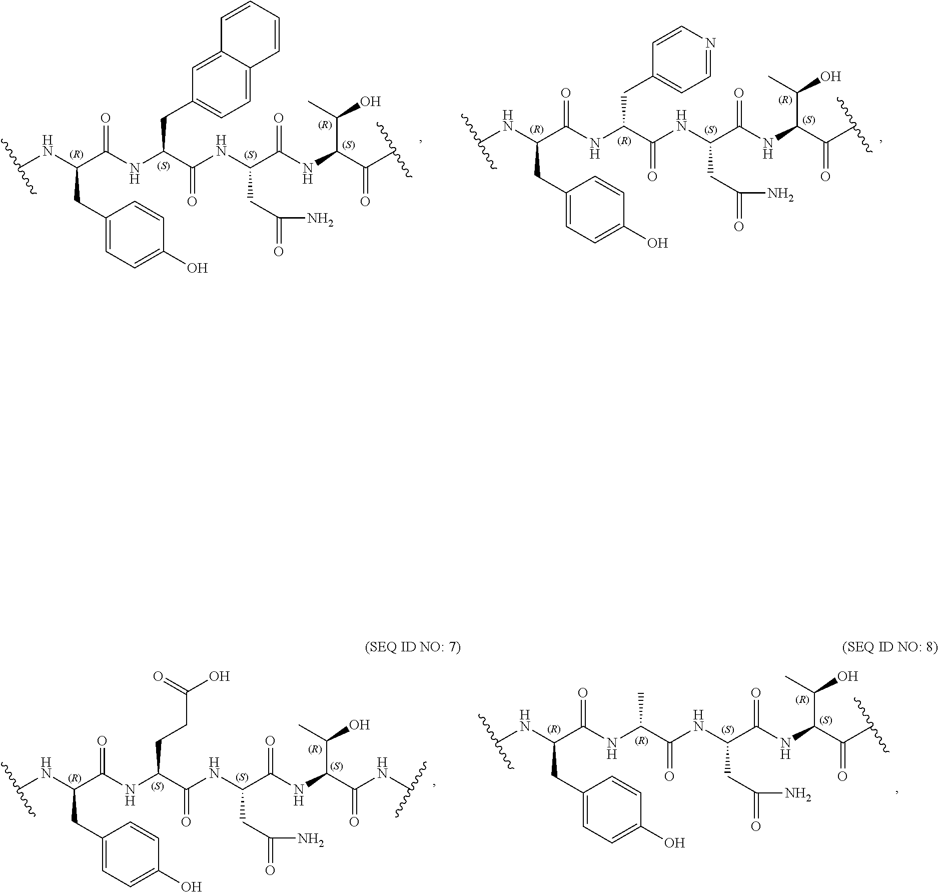

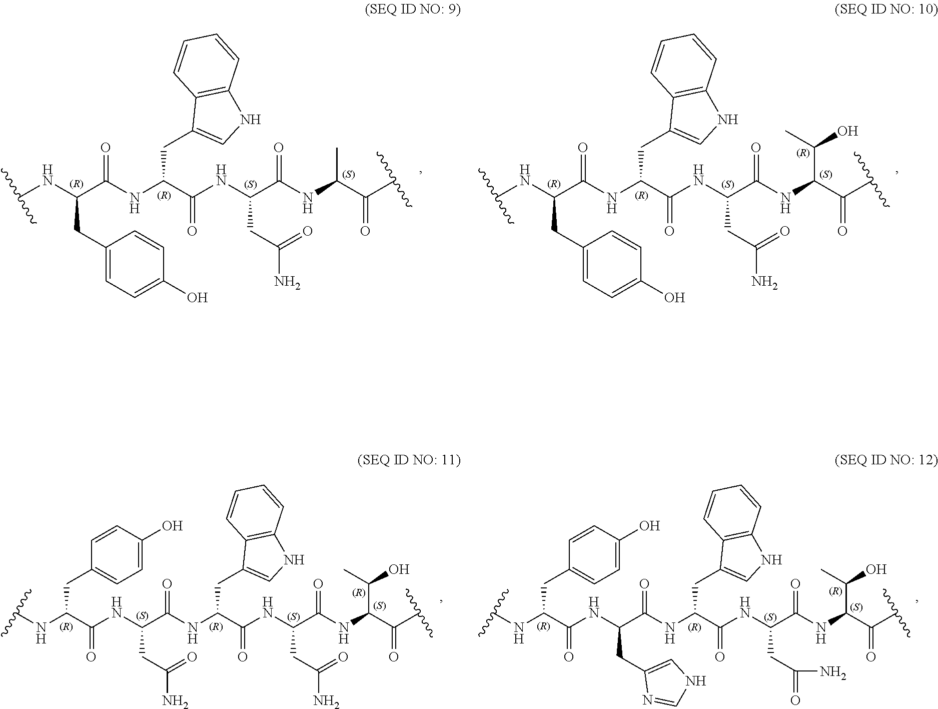

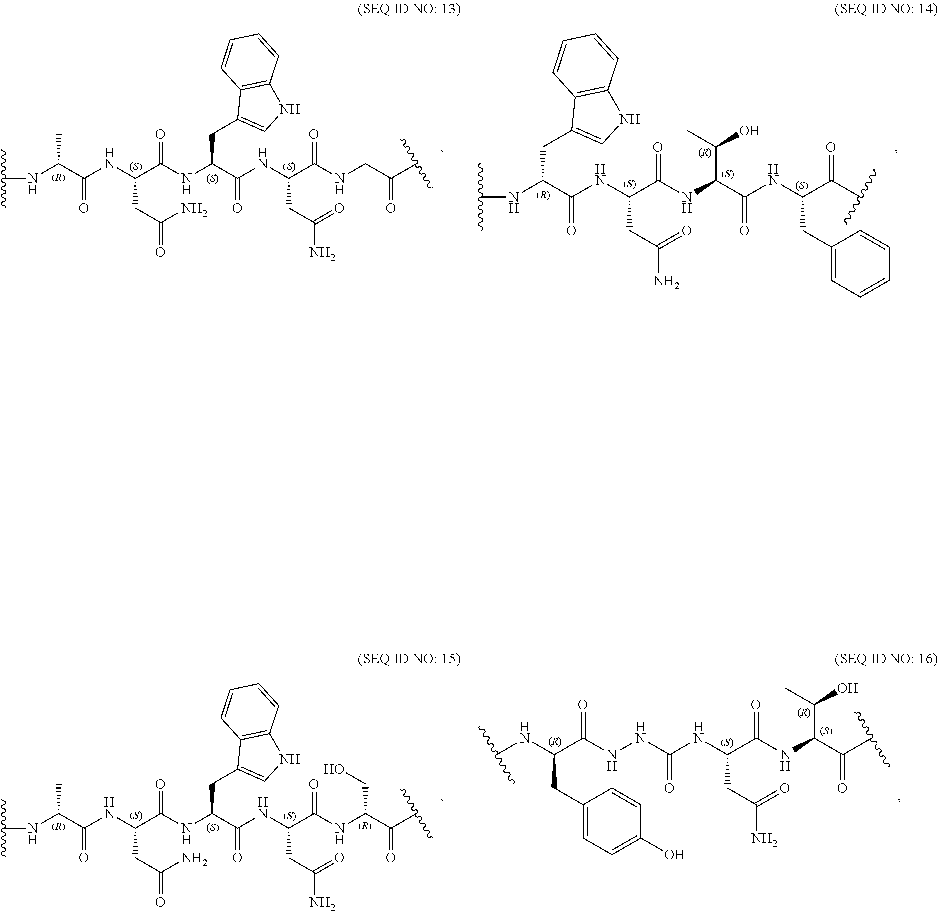

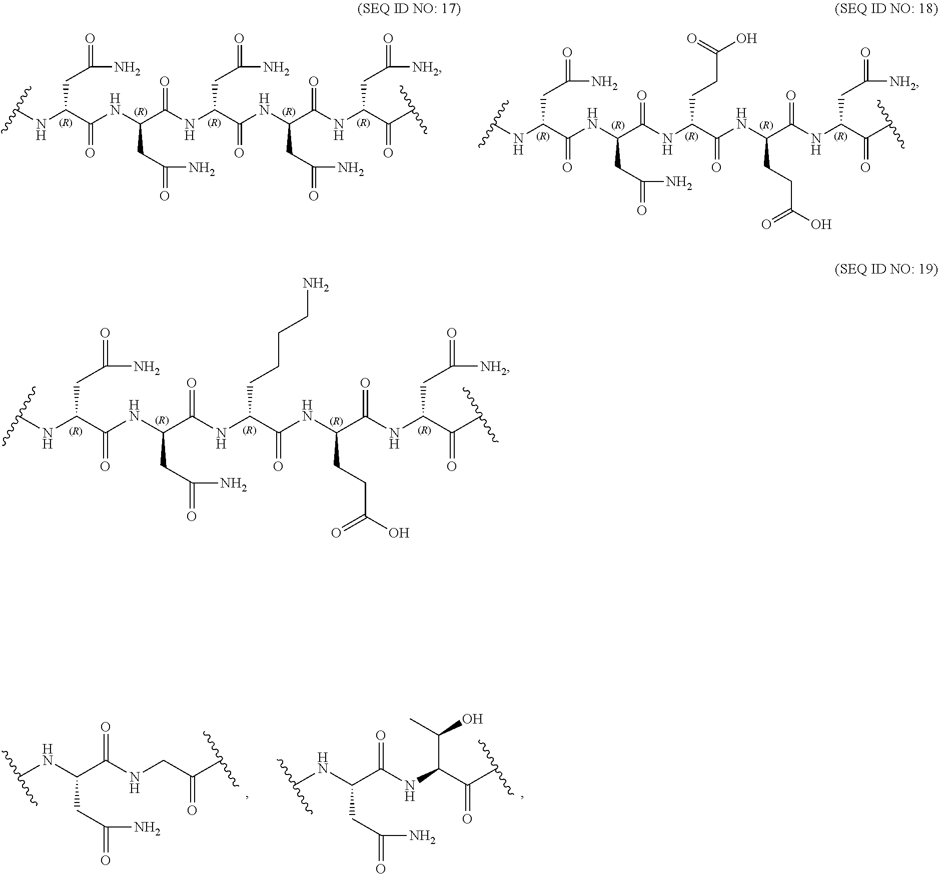

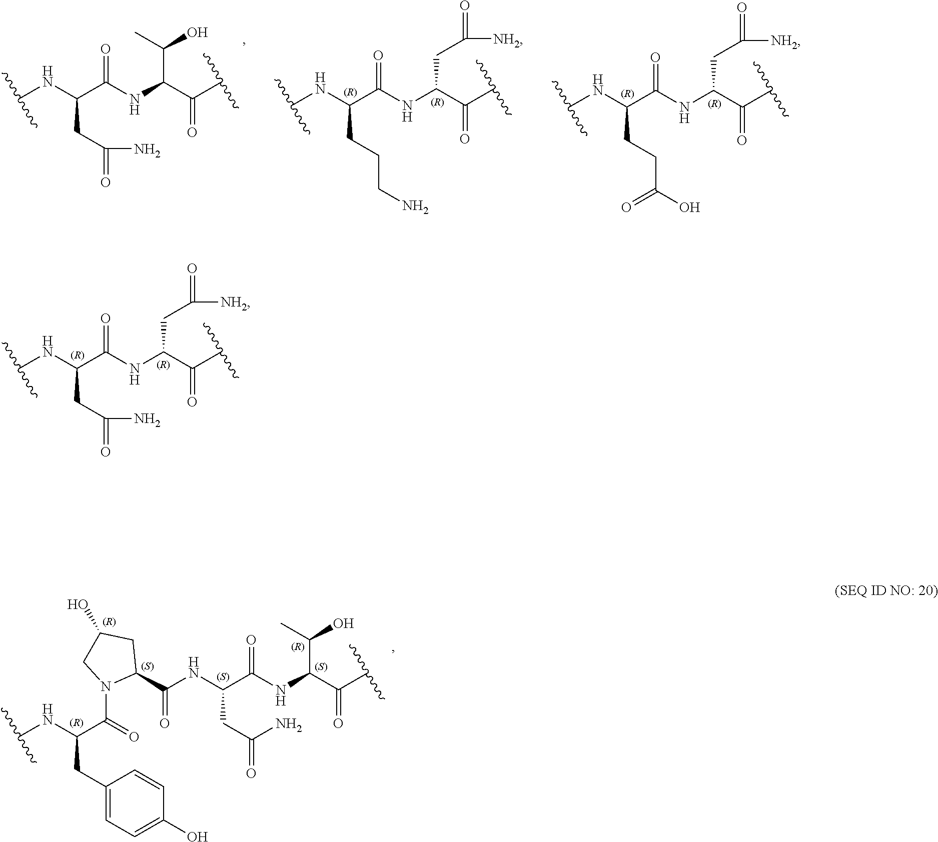

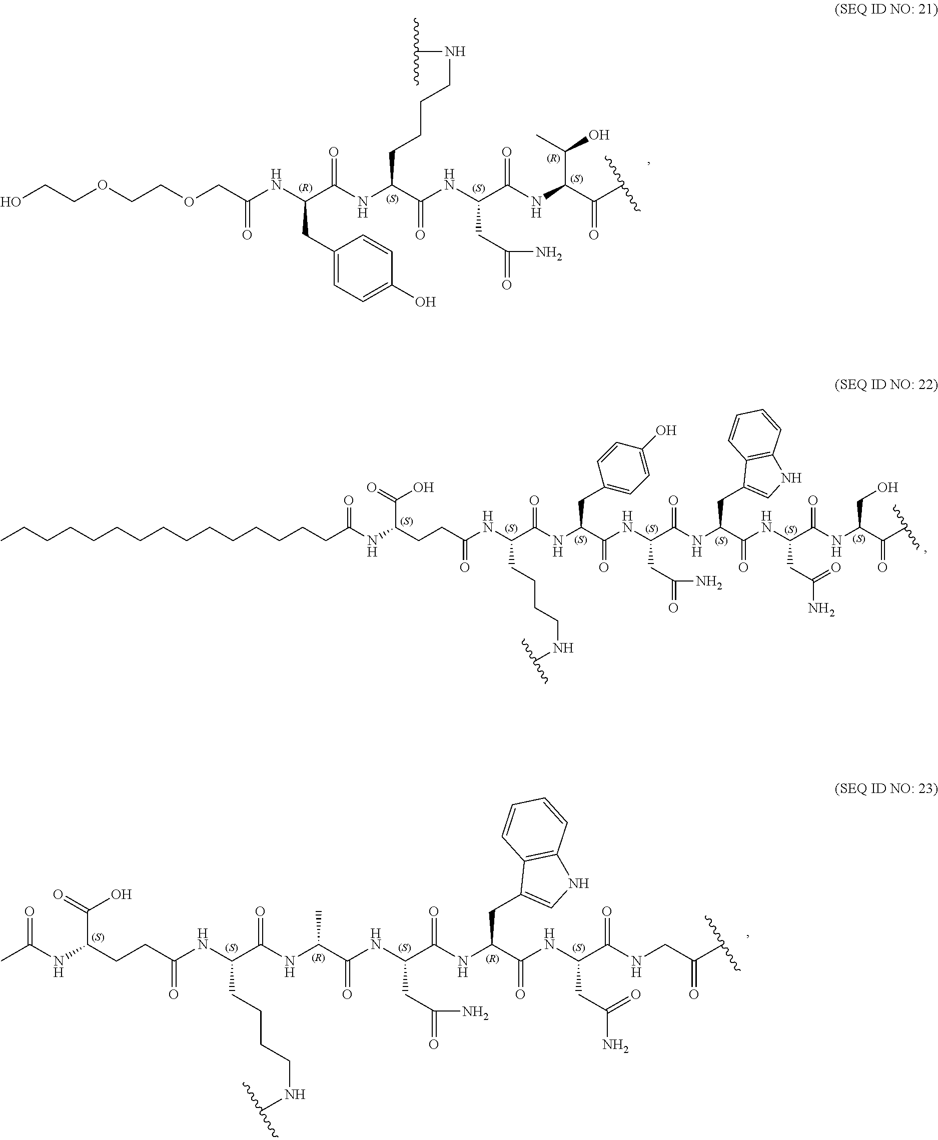

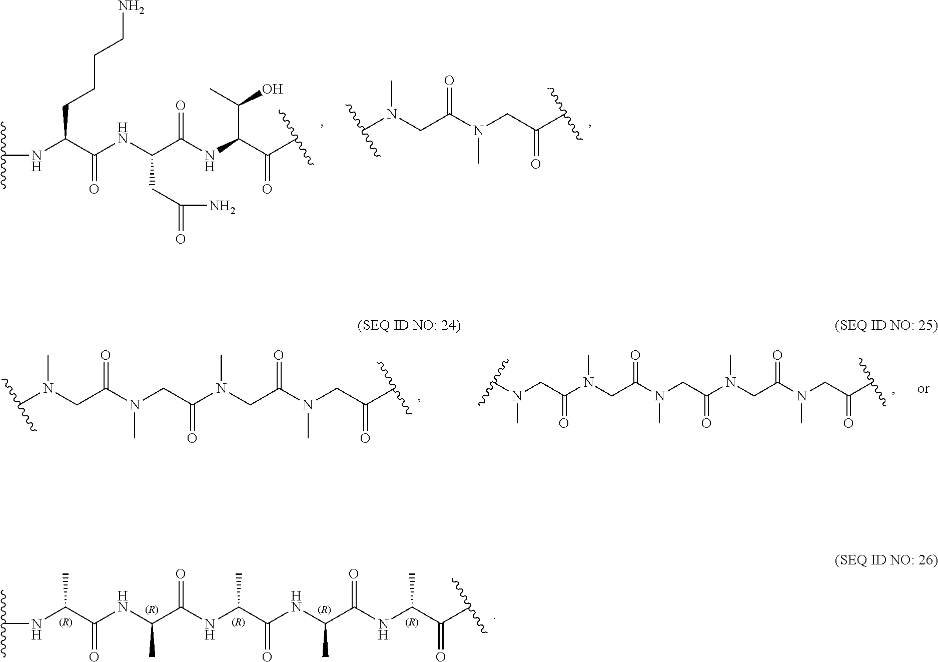

In some embodiments,

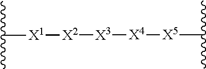

is absent, Asn-, -Lys-, -2-Pal-,-Thr-, -Trp-, -Asn-Asn-, -Asn-Gly-, -Asn-Thr-, -Glu-Asn-, -Glu-Thr-, -Lys-Asn-, -Lys(DOTA)-Asn-,-Lys-Thr-, -Lys(DOTA)-Thr-, -Lys-Glu-, -Lys(DOTA)-Glu-, -Sar-Sar-, -AAP-Asn-Thr-, -Asn-Phe-Thr-, -Glu-Asn-Thr-, -D-Glu-Asn-Thr-, -bGlu-Asn-Thr-, -Gly-Tyr-Ahx-, -Lys-Asn-Thr-,-D-β-Nal-Asn-Thr-, -D-4Pal-Asn-Thr-, -Thr-Asn-Arg-, -Trp-Asn-Thr-, -D-Trp-Asn-Thr-, -D-Tyr-Asn-Thr-, -Lys-Asn-Thr-, -Lys(DOTA)-Asn-Thr-, -Gly-Tyr-β-Nal-Ahx-, -Lys-Trp-Asn-Thr-(SEQ 1) NO: 28), -Phe(4-I)-Trp-Asn-Thr- (SEQ ID NO: 29), -D-Phe(4-I)-D-Trp-Asn-Thr-(SEQ ID NO: 30), -Sar-Sar-Sar-Sar- (SEQ ID NO: 24), D-Trp-Asn-Thr-Phe- (SEQ ID NO: 14), -D-Tyr-AAP-Asn-Thr-, -D-Tyr-Arg-Asn-Thr- (SEQ ID NO: 3), -D-Tyr-D-Ala-Asn-Thr- (SEQ ID NO: 8), -Tyr-AzaGly-Asn-Thr- (SEQ ID NO: 32), -D-Tyr-AzaGly-Asn-Thr- (SEQ ID NO: 16), -D-Tyr-Bip-Asn-Thr-, -D-Tyr-Bpa-Asn-Thr-, -D-Tyr-Glu-Asn-Thr- (SEQ ID NO: 7), -D-Tyr-Hyp-Asn-Thr-,-D-Tyr-D-Hyp-Asn-Thr-,-Tyr-Lys-Asn-Thr- (SEQ ID NO: 4), -D-Tyr-Lys(DOTA)-Asn-Thr- (SEQ ID NO: 38), HO—(CH₂CH₂O)₂—CH₂C(═O)-D-Tyr-Lys(DOTA)-Asn-Thr-(SEQ ID NO: 845), -Tyr-D-Lys-Asn-Thr- (SEQ ID NO: 829), -Tyr-D-Lys(DOTA)-Asn-Thr-(SEQ ID NO: 39), -D-Tyr-D-Lys-Asn-Thr- (SEQ ID NO: 830), -D-Tyr-D-Lys(DOTA)-Asn-Thr-(SEQ ID NO: 40), -Tyr-β-Nal-Asn-Thr-, -D-Tyr-r-Nal-Asn-Thr-, -D-Tyr-D-β- Nal-Asn-Thr-, -Tyr-4Pal-Asn-Thr-, -D-Tyr-4Pal-Asn-Thr-, -D-Tyr-D-4Pal-Asn-Thr-, -D-Tyr-Phe(4-I)-Asn-Thr- (SEQ ID NO: 45), -Tyr-Pro-Asn-Thr- (SEQ ID NO: 46), -D-Tyr-Pro-Asn-Thr-(SEQ ID NO: 20), D-Tyr-Trp-Asn-Ala- (SEQ ID NO: 9), -D-Tyr-D-Trp-Asn-Ala-(SEQ ID NO: 47), -D-Tyr-Trp-Asn-Thr- (SEQ ID NO: 10), -D-Tyr-D-Trp-Asn-Thr- (SEQ ID NO: 1), -D-Ala-D-Ala-D-Ala-D-Ala-D-Ala- (SEQ ID NO: 26), D-Ala-Asn-Trp-Asn-Gly- (SEQ ID NO: 13), D-Ala-Asn-Trp-Asn-D-Ser (SEQ ID NO: 15), -D-Asn-)-Asn-D-Asn-D-Asn-D-Asn-(SEQ ID NO: 17), -D-Asn-D-Asn-D-Glu-D-Glu-D-Asn- (SEQ ID NO: 18), -D-Asn-D-Asn-D-Lys-D-Glu-D-Asn-(SEQ ID NO: 19), -γ-D-Glu-D-Tyr-Lys(DOTA)-Asn-Thr- (SEQ ID NO: 48) -Gly-D-Tyr-β-Nal-Asn-Thr- (SEQ ID NO: 49), -Gly-Tyr-D-Trp-Asn-Thr- (SEQ ID NO: 50), -Sar-Sar-Sar-Sar-Sar- (SEQ ID NO: 25), -Tyr-Asn-Trp-Asn-Ser- (SEQ ID NO: 51), -D-Tyr-D-Asn-D-Arg-Asn-Thr-(SEQ ID NO: 2), -D-Tyr-Asn-D-Trp-Asn-Thr (SEQ ID NO: 11), -Tyr-Glu-Asn-Thr-3-F-Phe- (SEQ ID NO: 52), -D-Tyr-D-Asn-D-Trp-Asn-Thr- (SEQ ID NO: 12), -Tyr-D-Trp-Asn-Thr-3-F-Phe- (SEQ ID NO: 53), -D-Tyr-D-Trp-Asn-Thr-3-F-Phe- (SEQ ID NO: 54), Palmitic Acid-γGlu-Lys(DOTA)-Tyr-Asn-Trp-Asn-Ser- (SEQ ID NO: 846), or Ac-γGlu-Lys(DOTA)-D-Ala-Asn-Trp-Asn-Gly-(SEQ ID NO: 847).

In some embodiments,

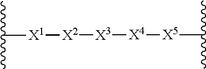

is

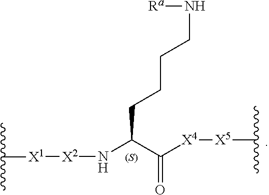

In some embodiments,

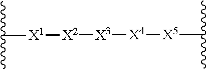

is

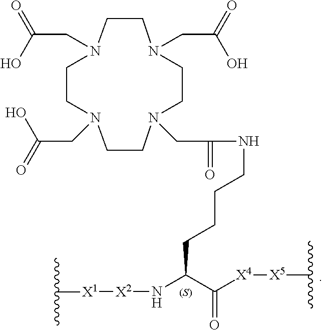

In some embodiments,

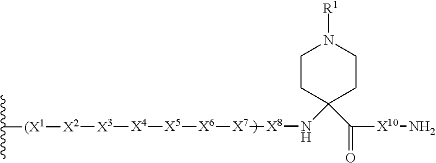

is

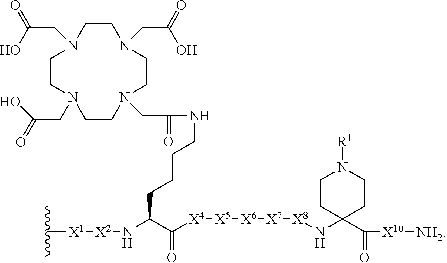

In some embodiments,

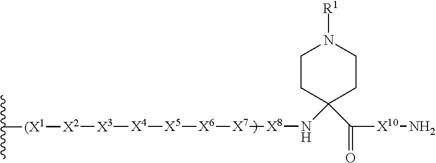

is

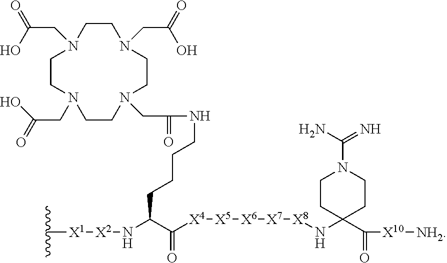

In some embodiments,

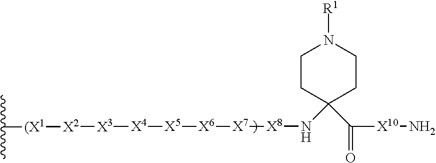

is

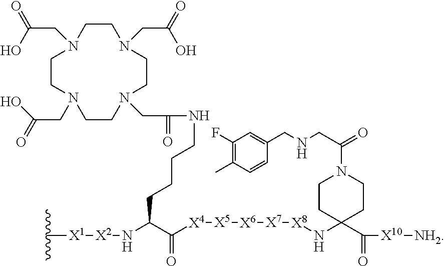

In some embodiments,

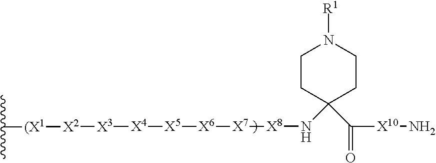

is

[chemical structure]

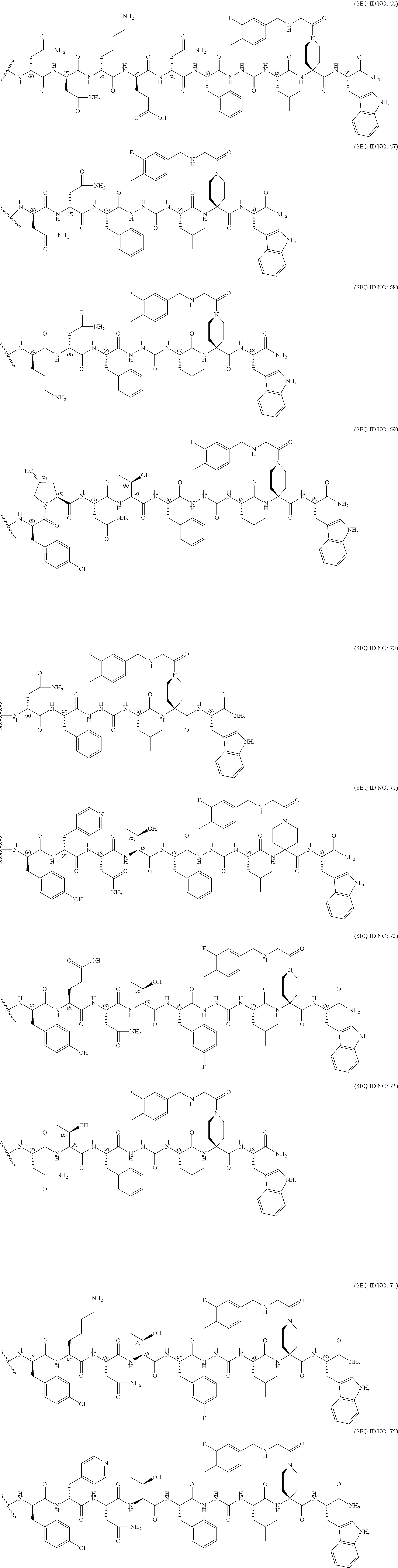

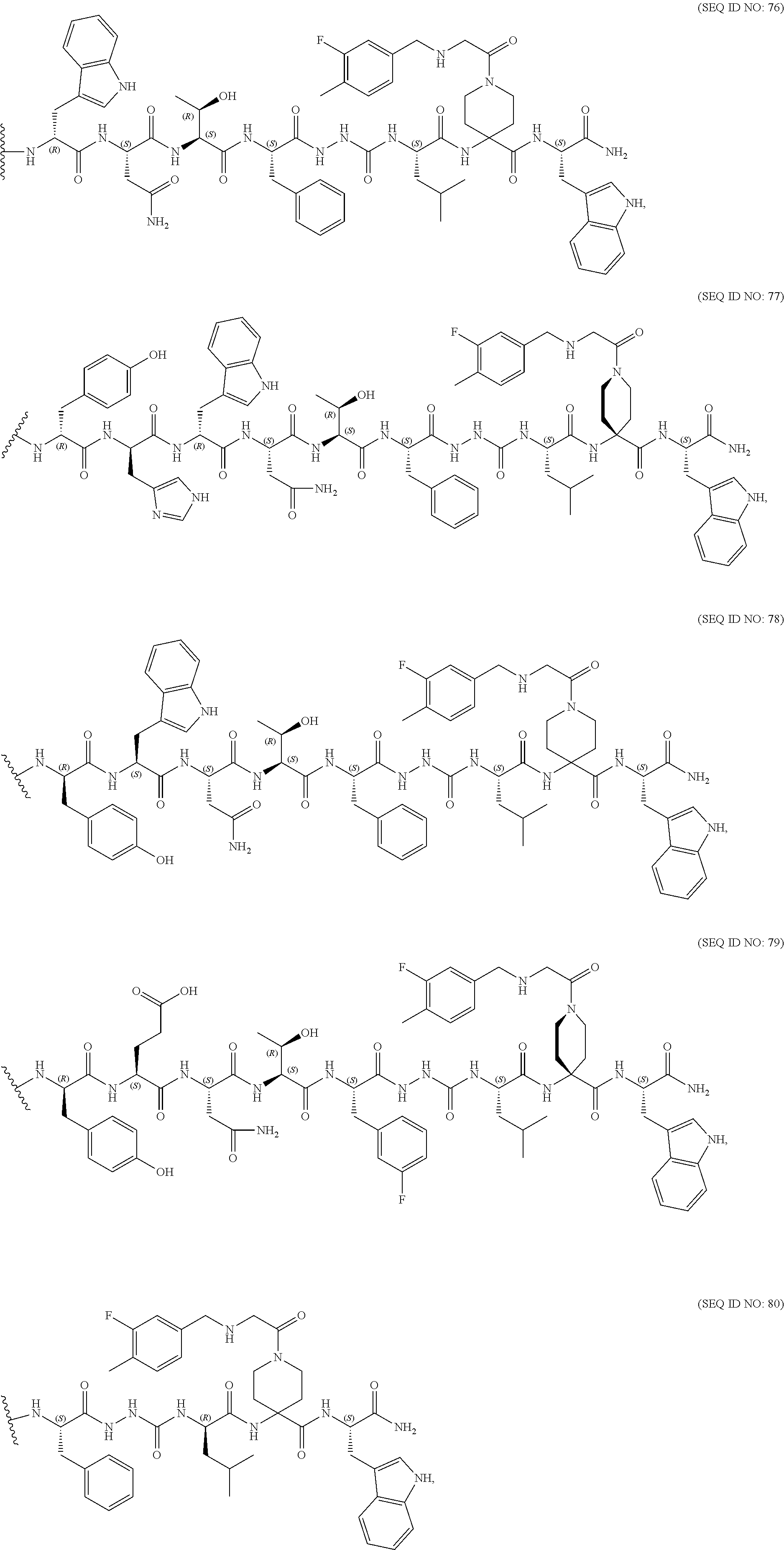

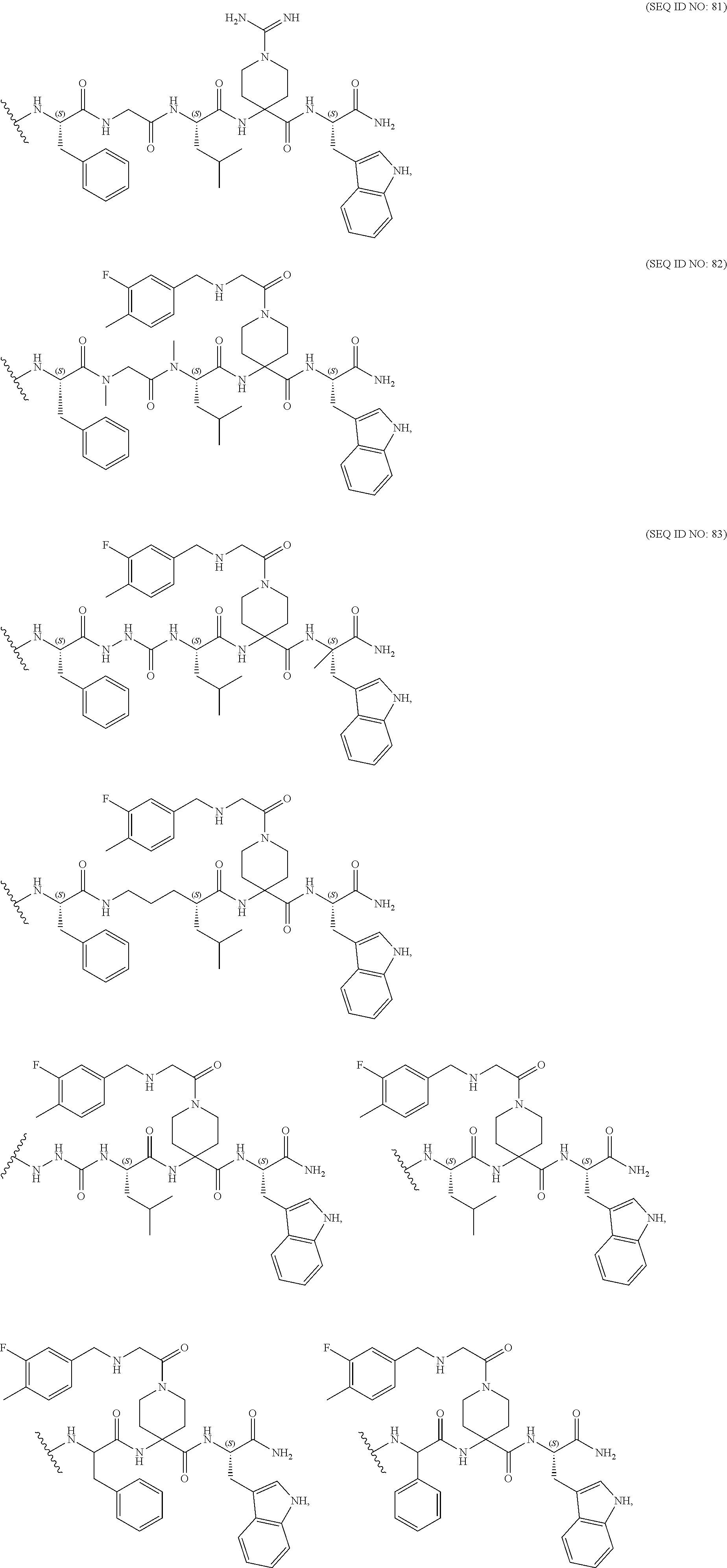

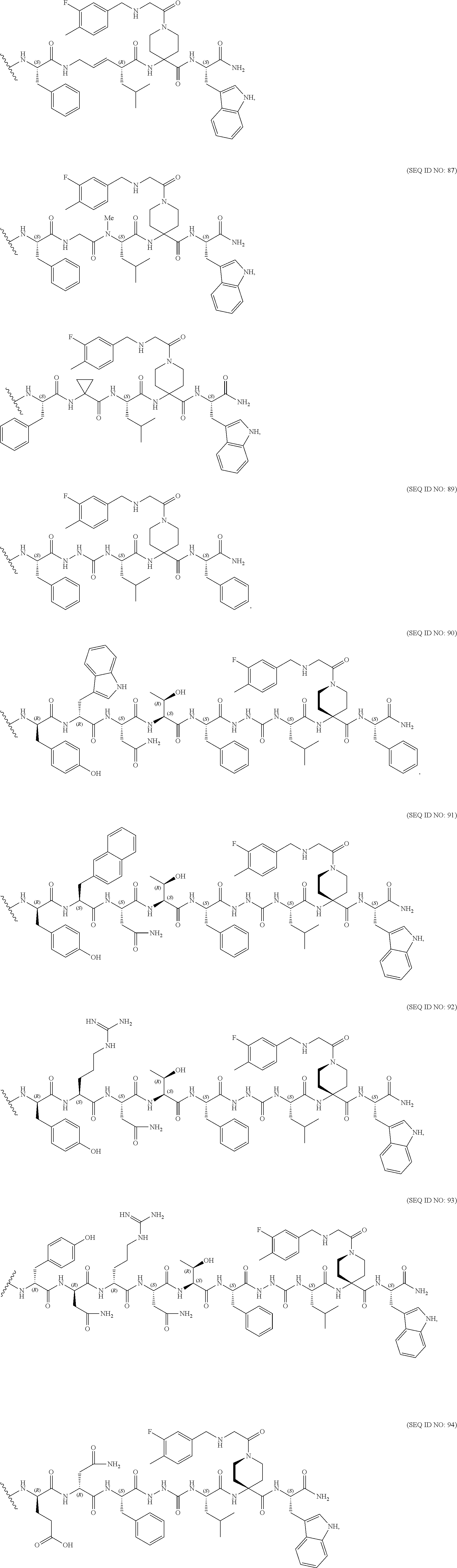

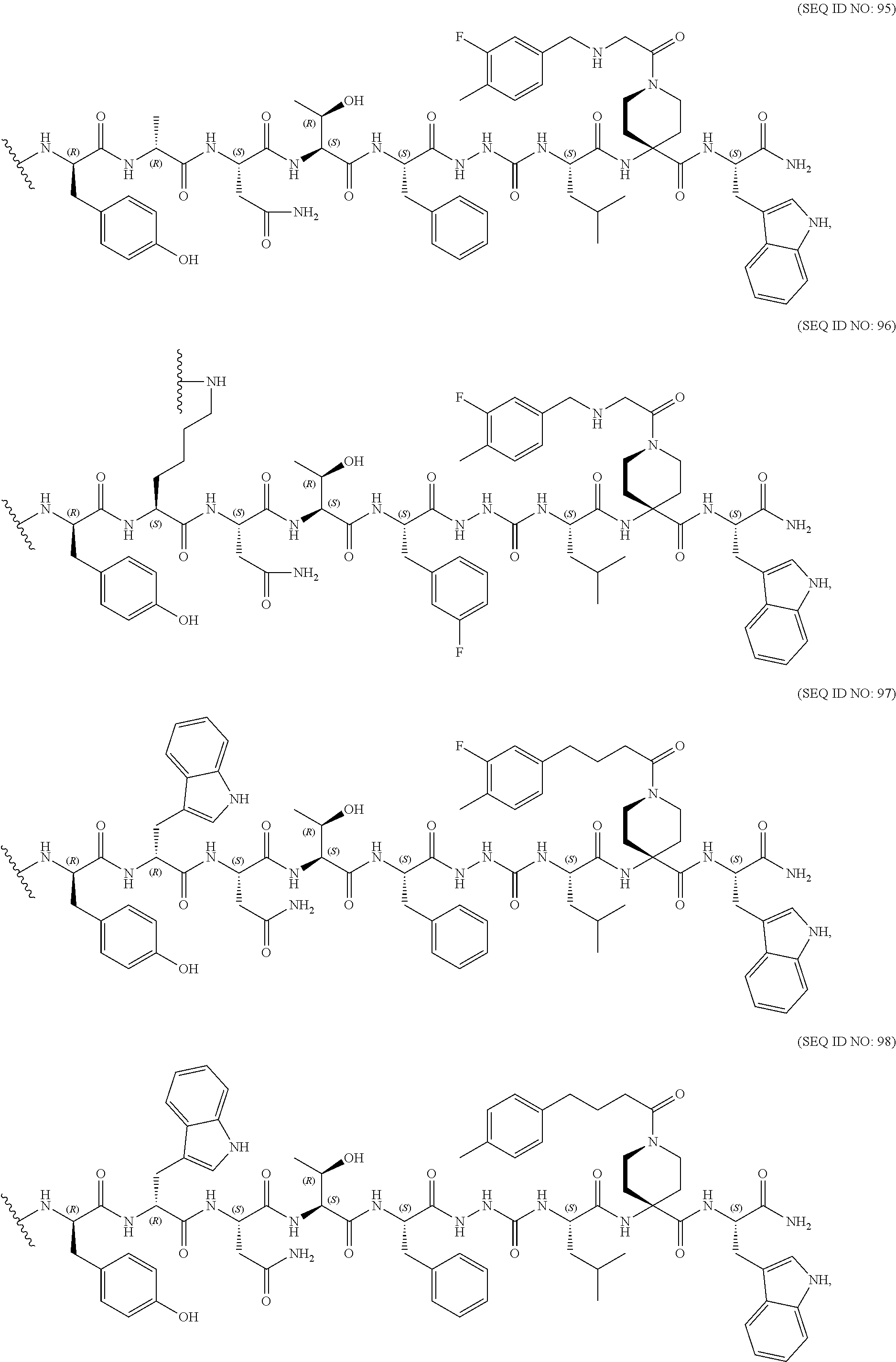

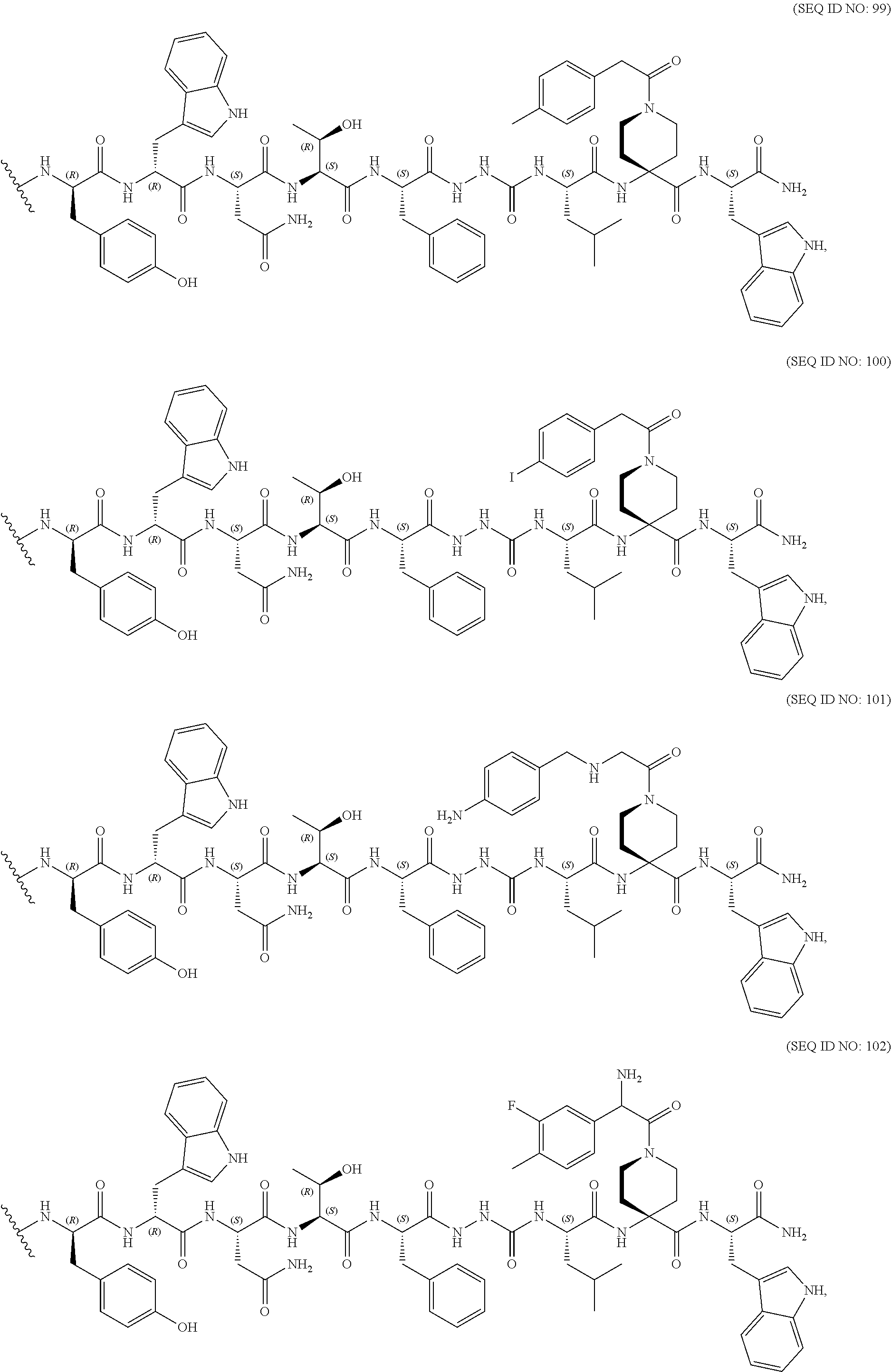

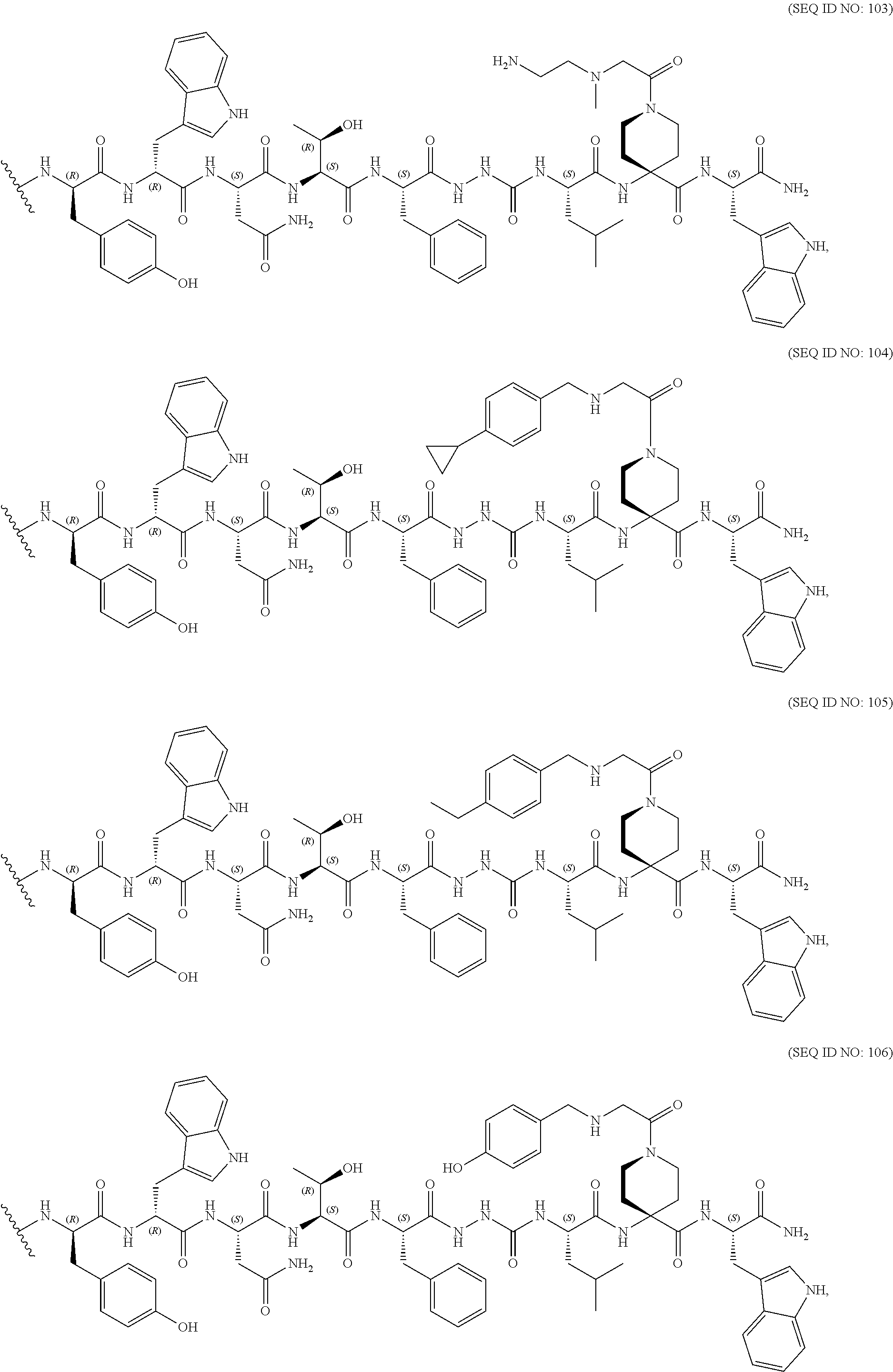

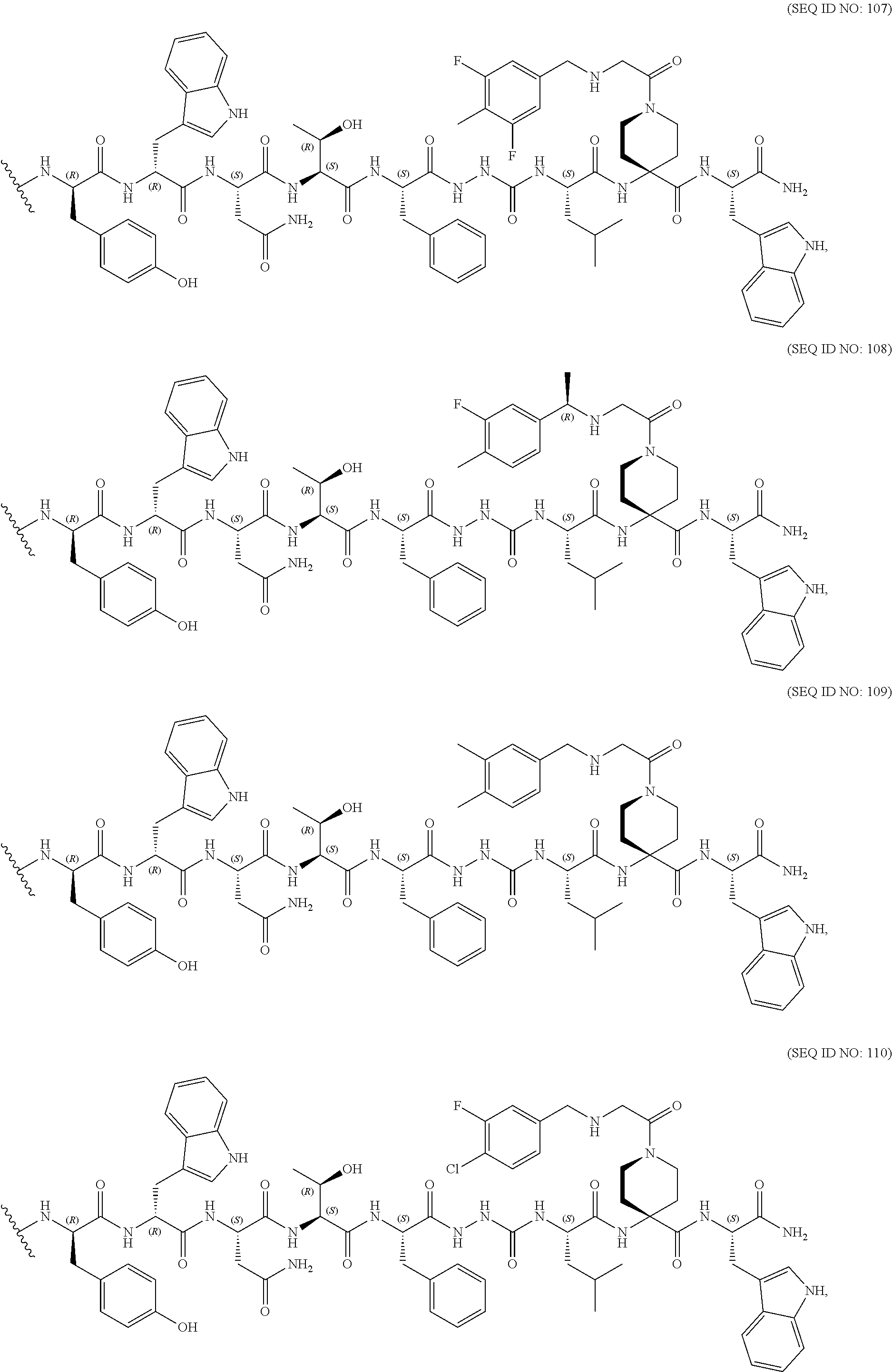

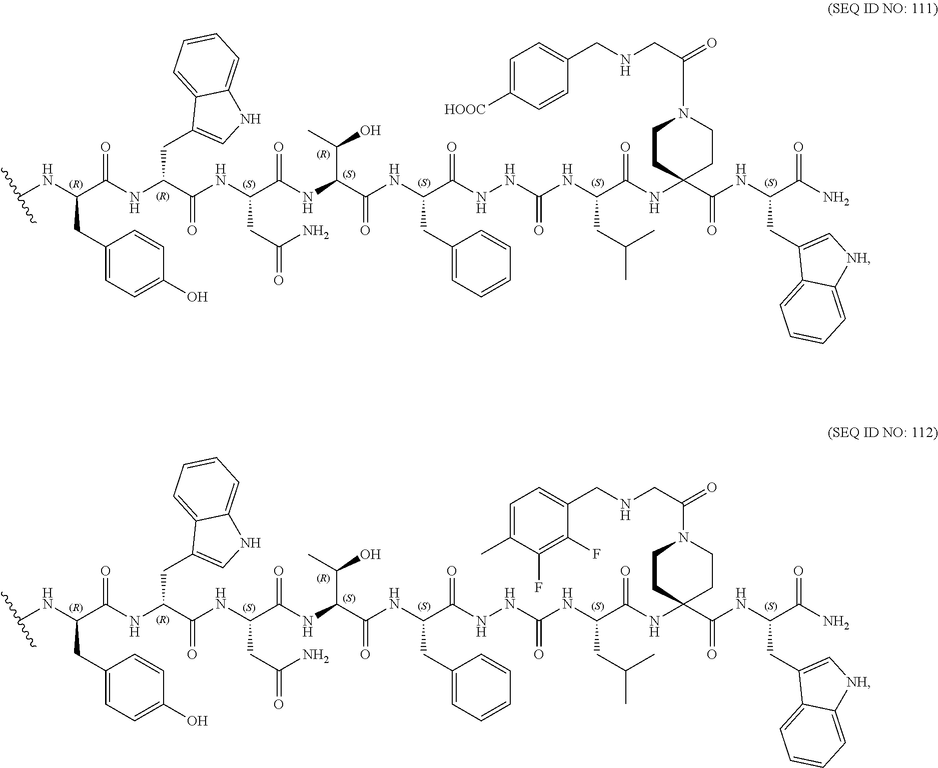

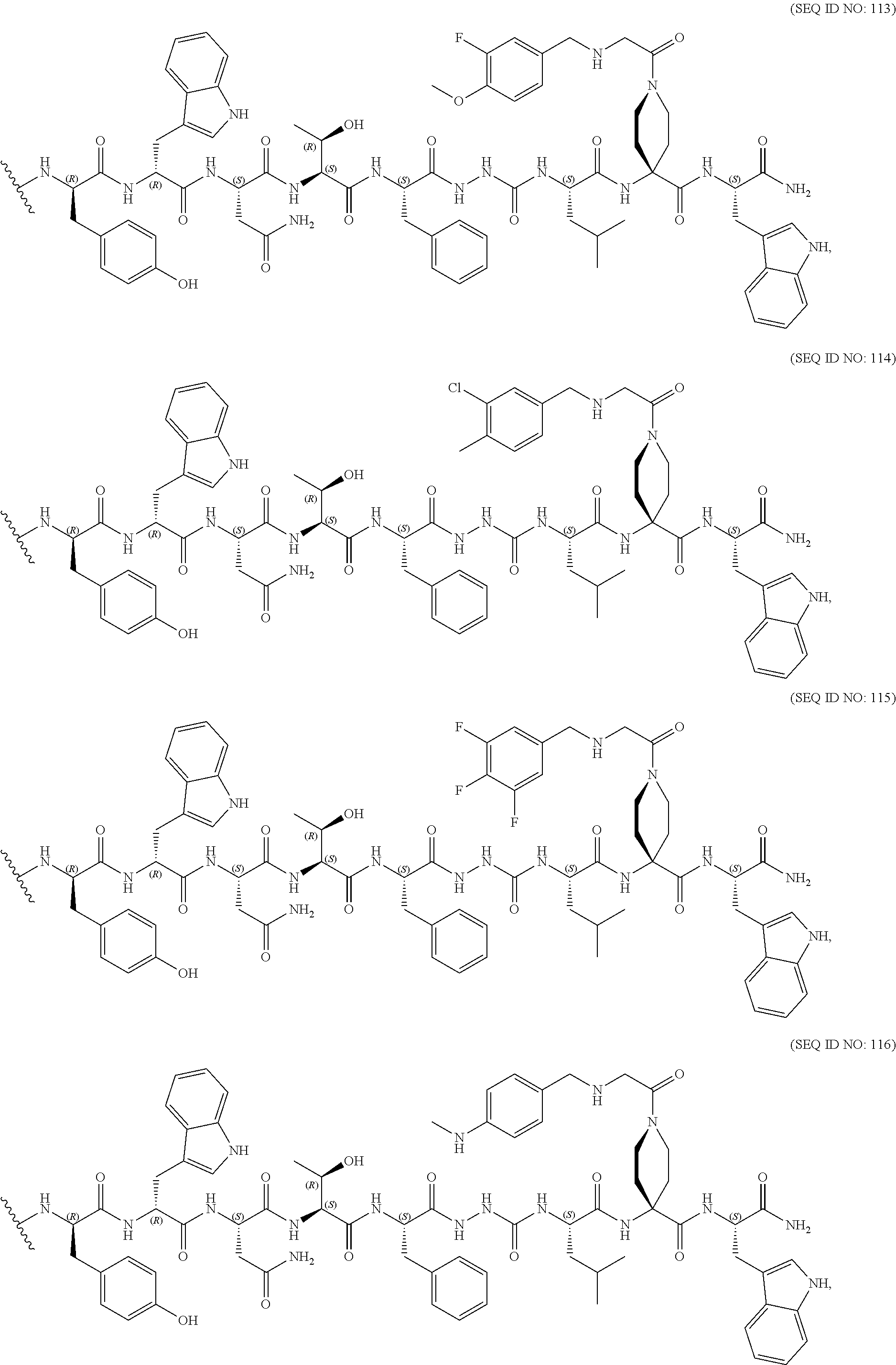

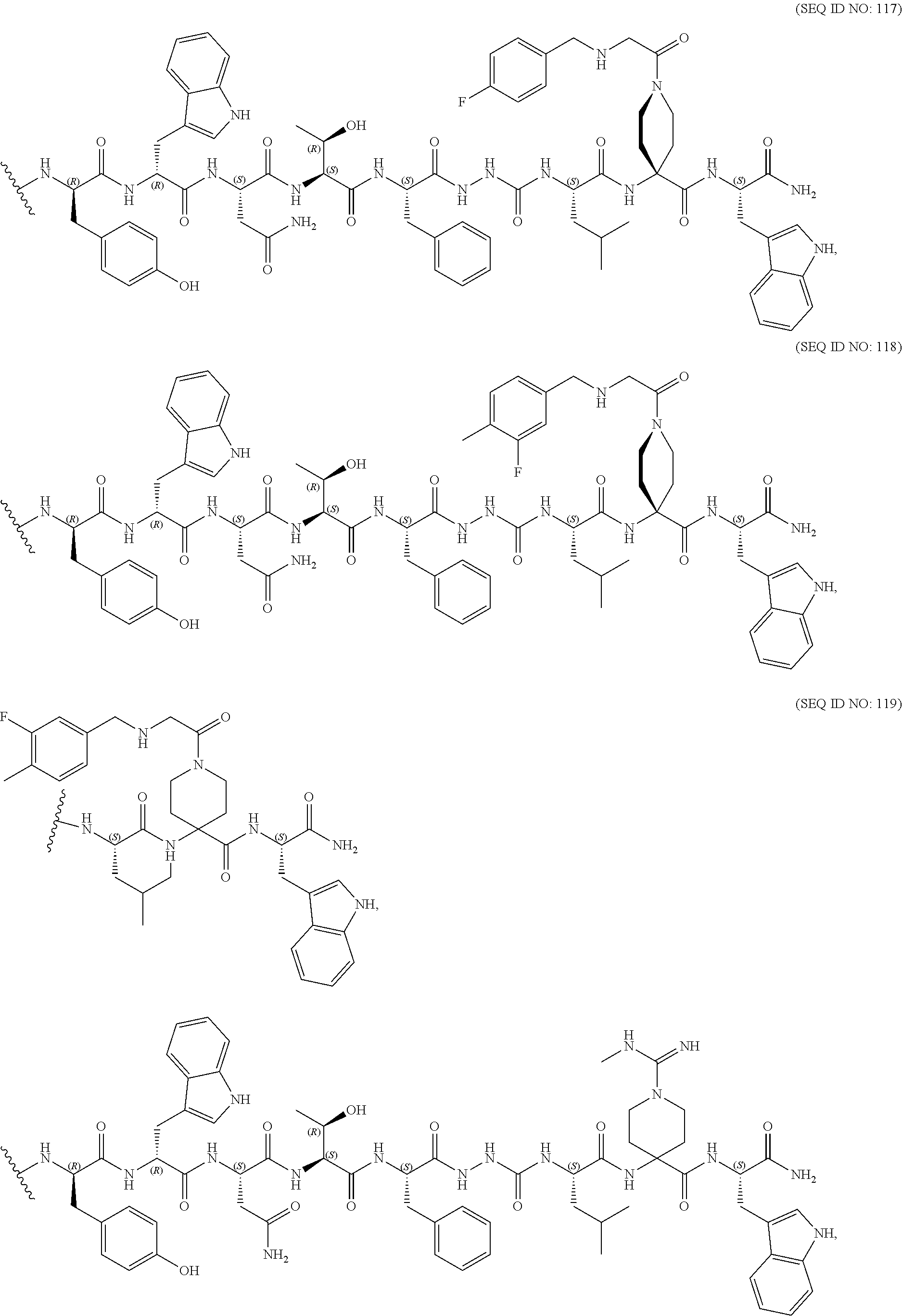

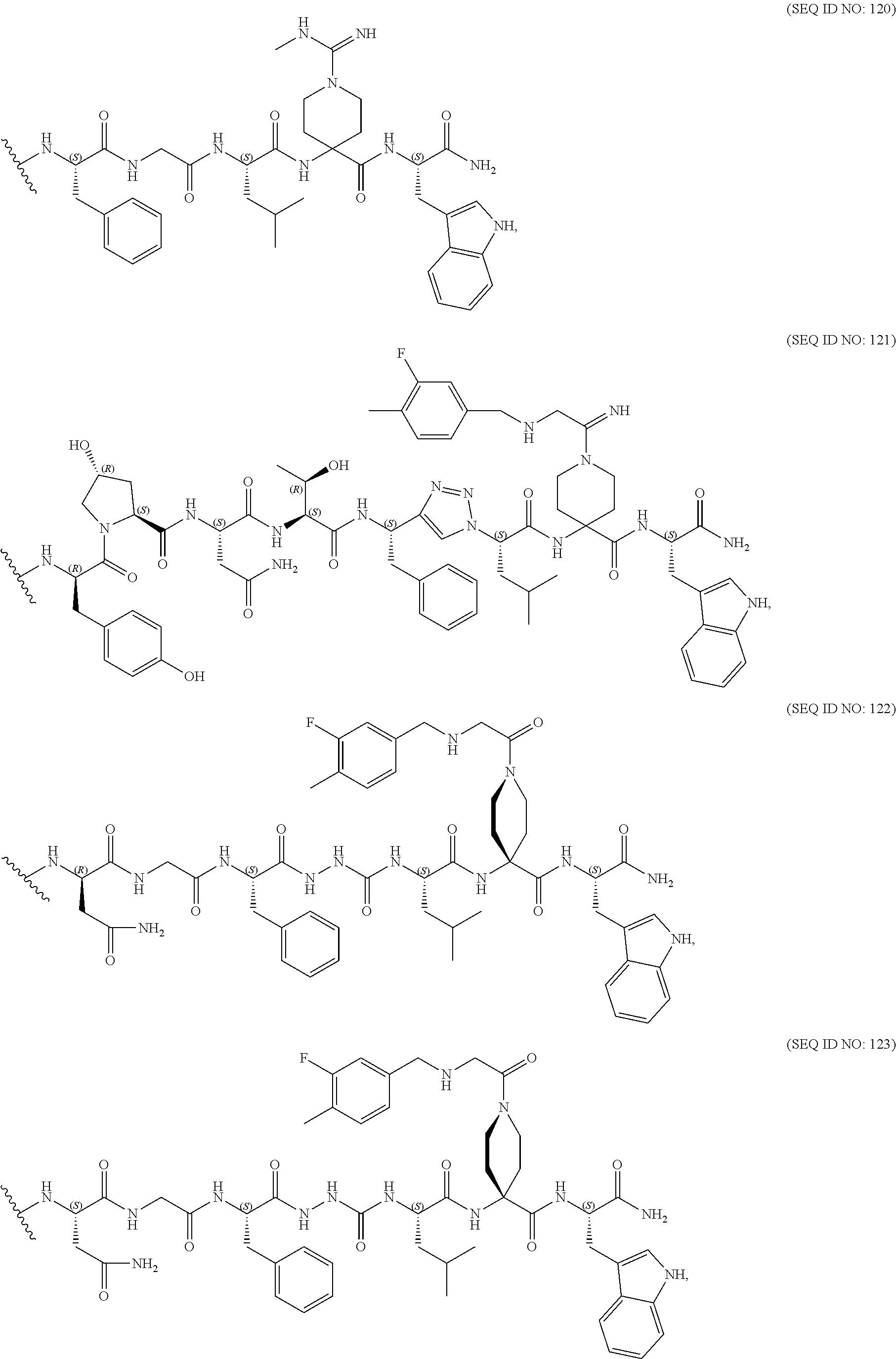

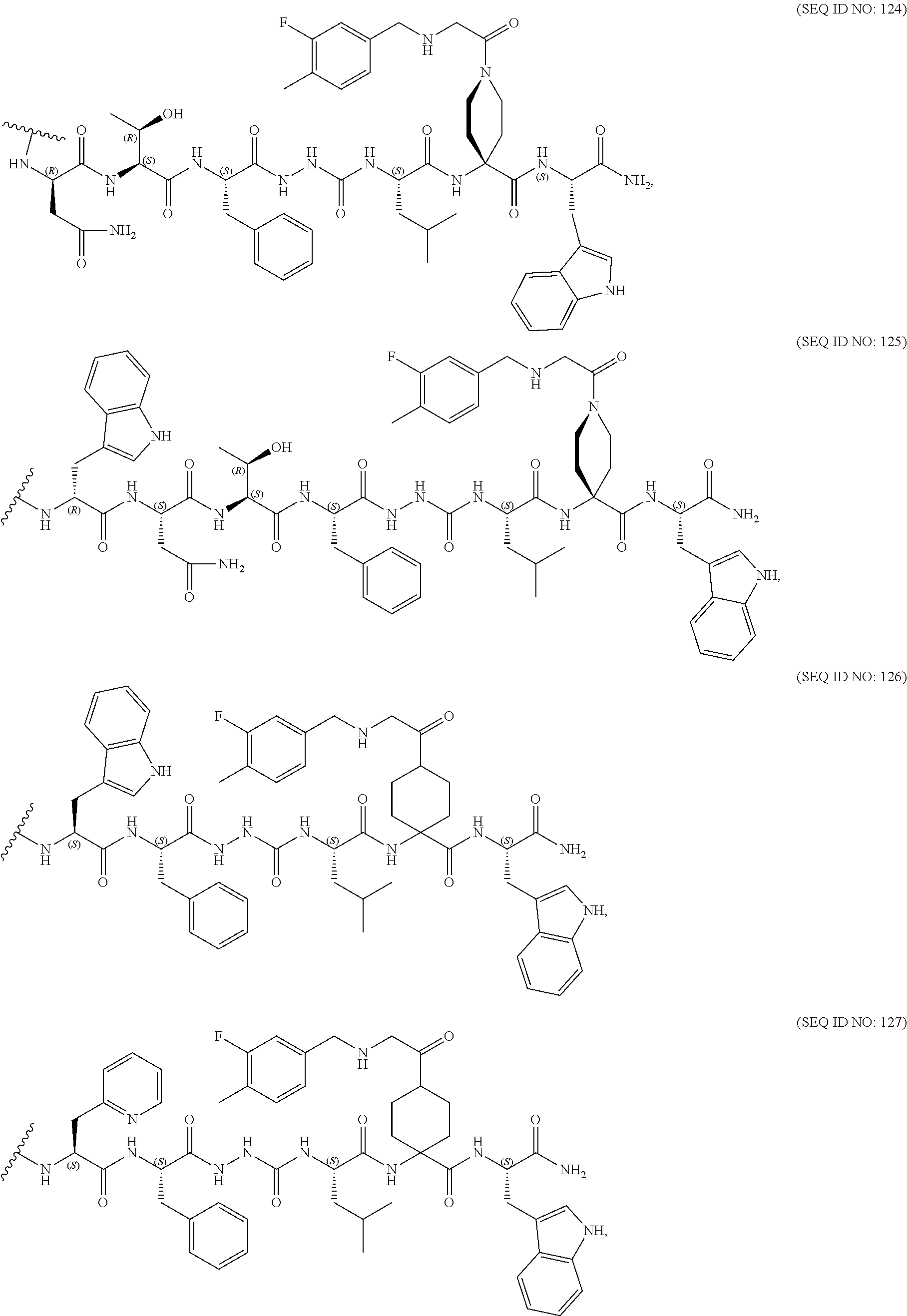

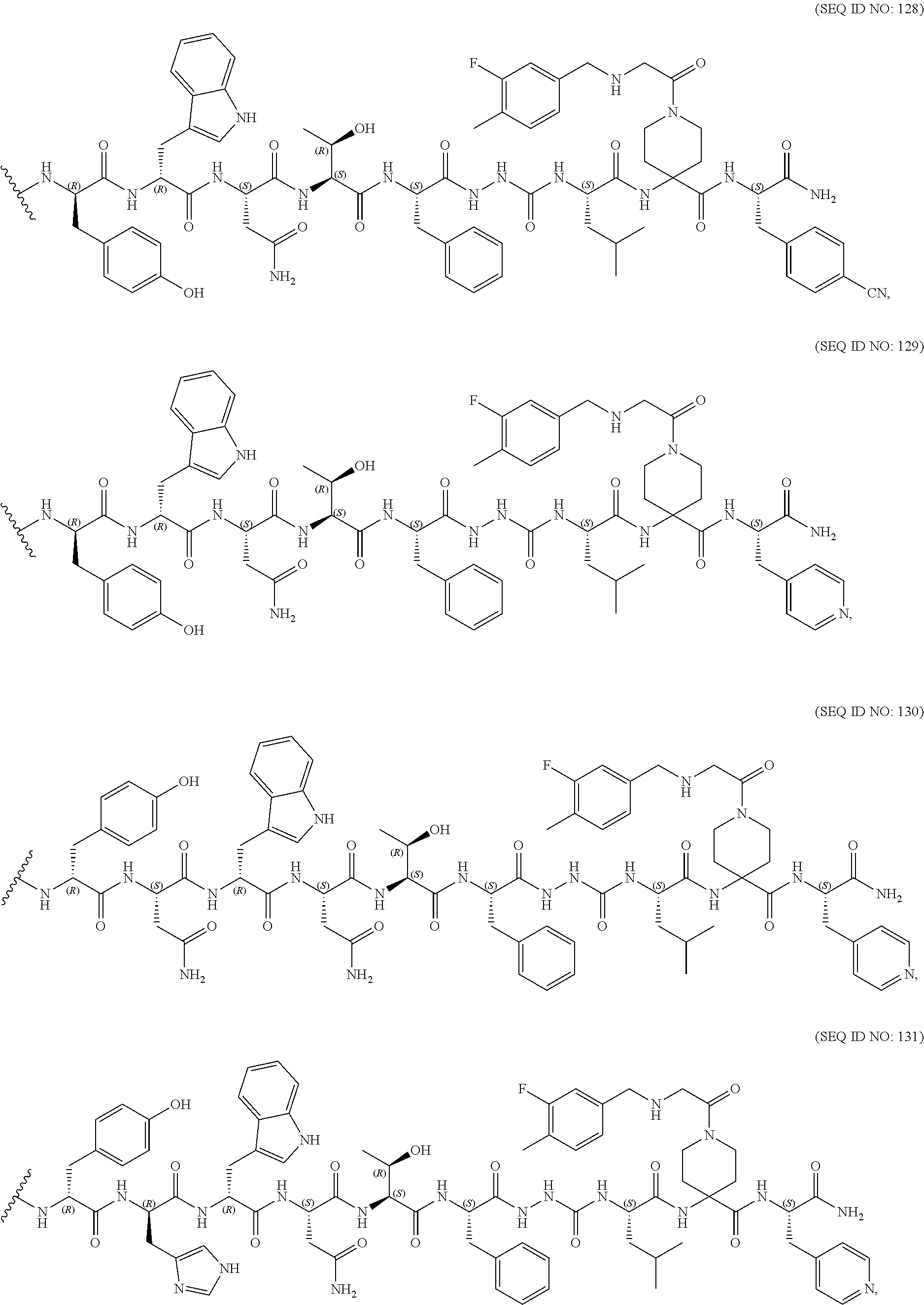

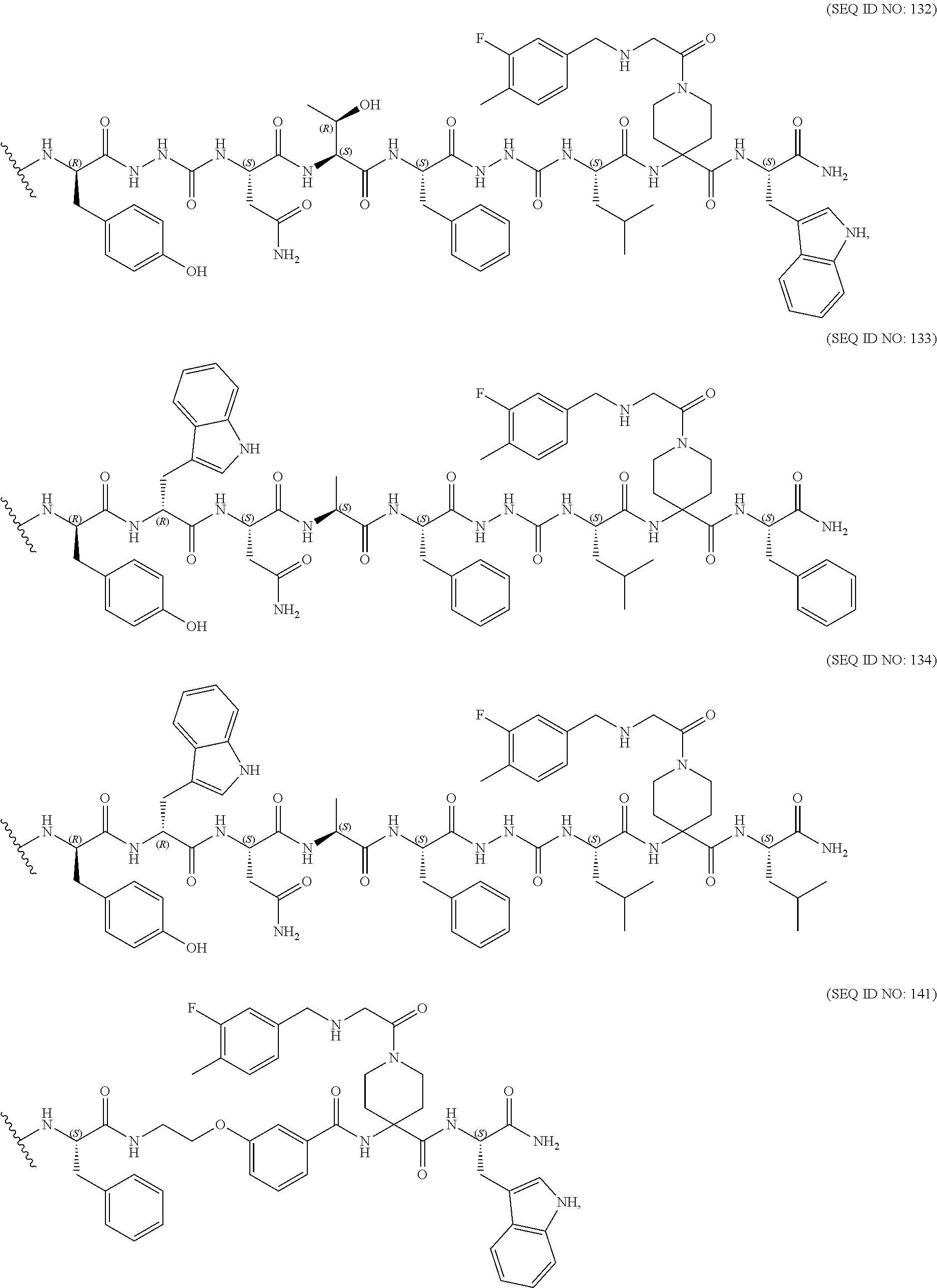

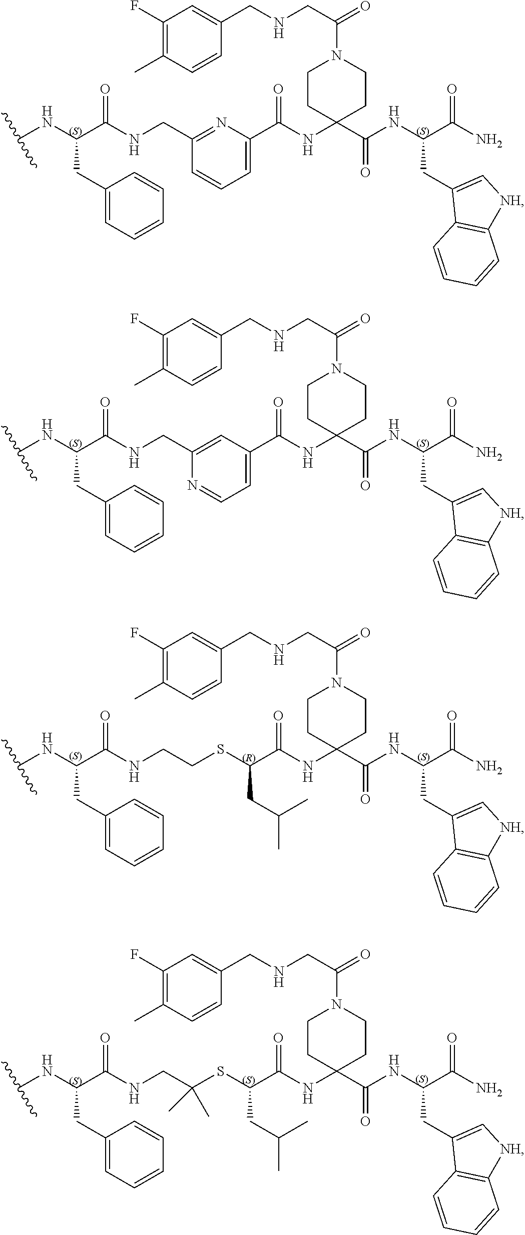

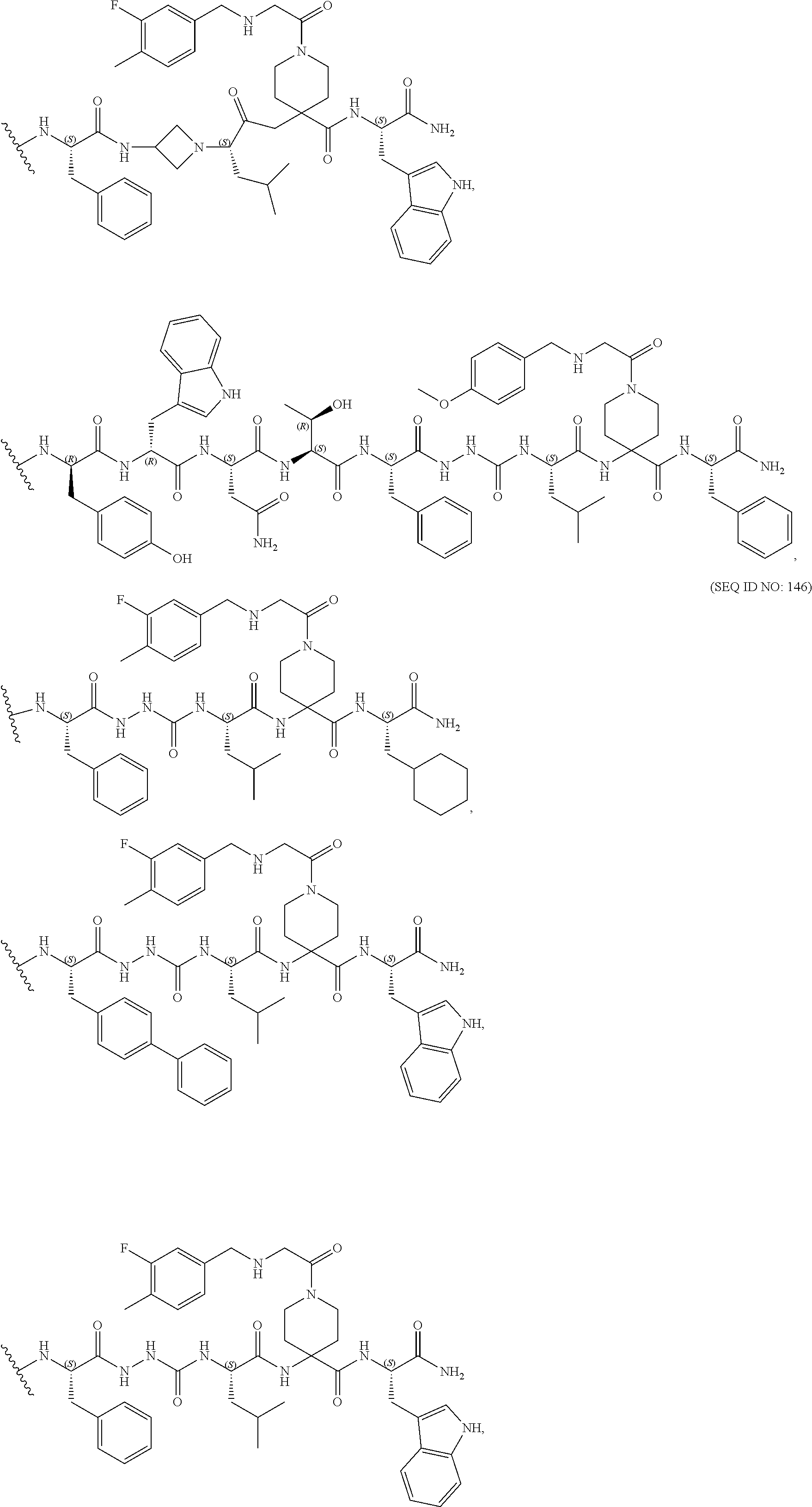

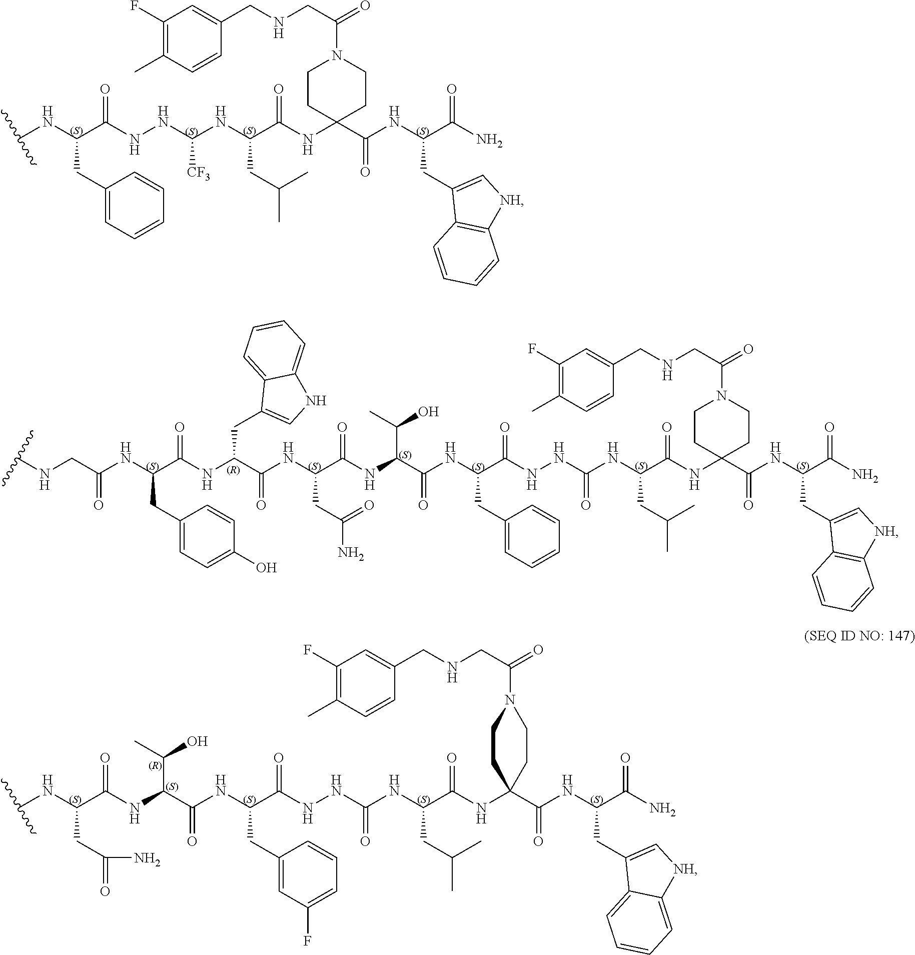

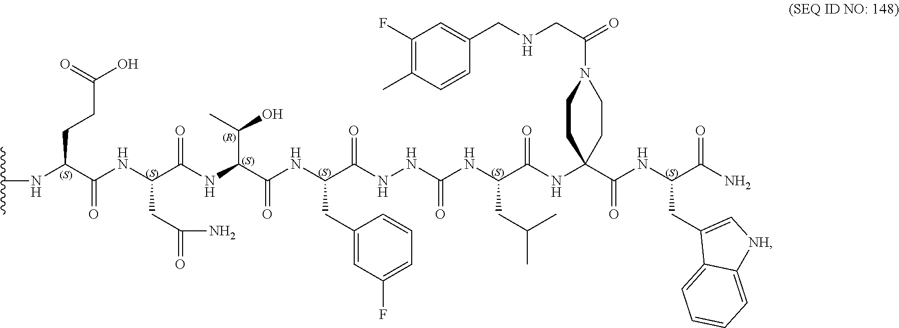

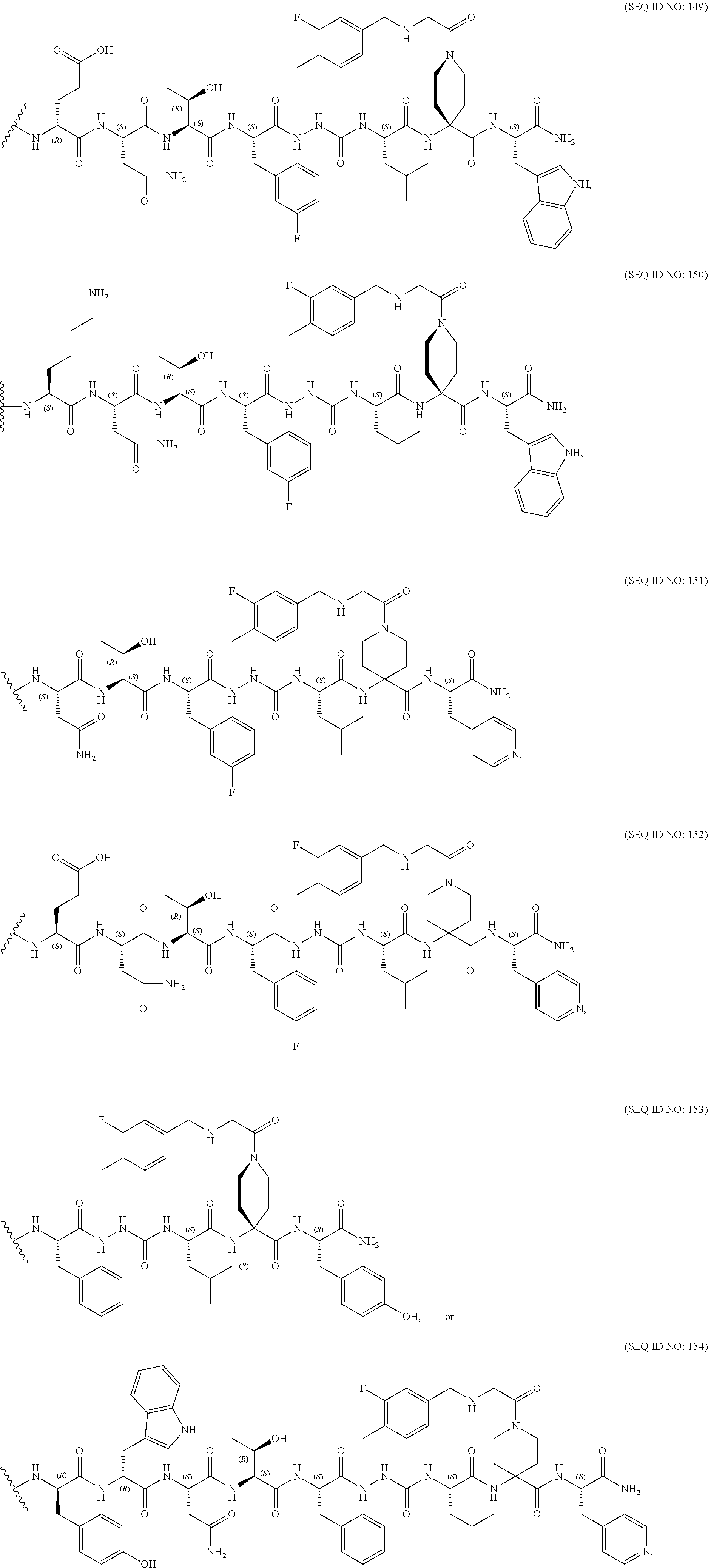

In some embodiments,

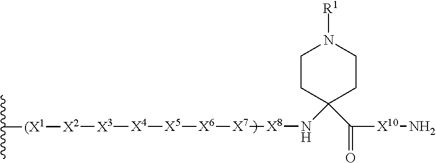

is

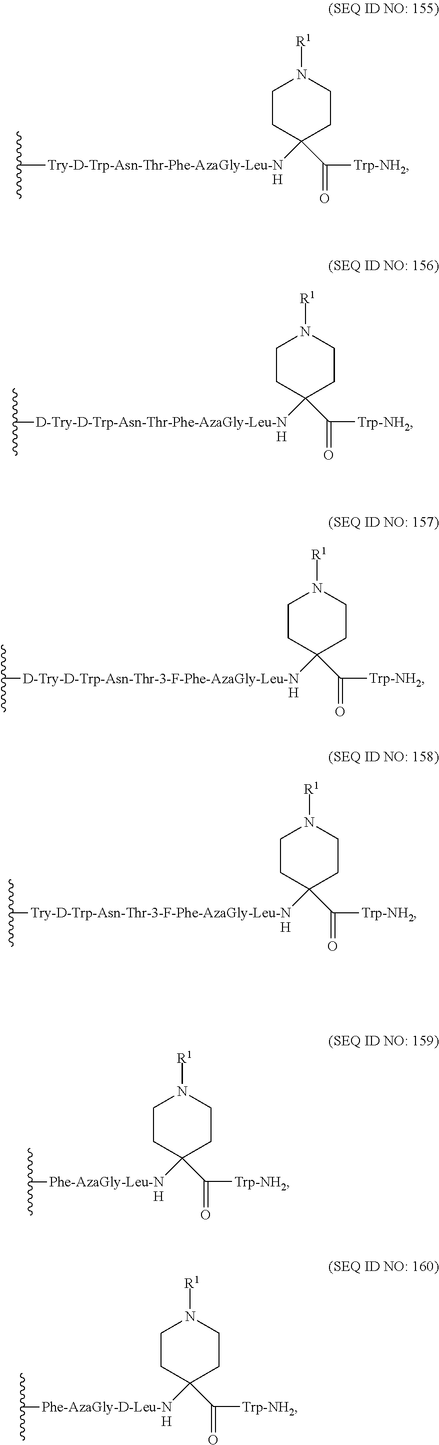

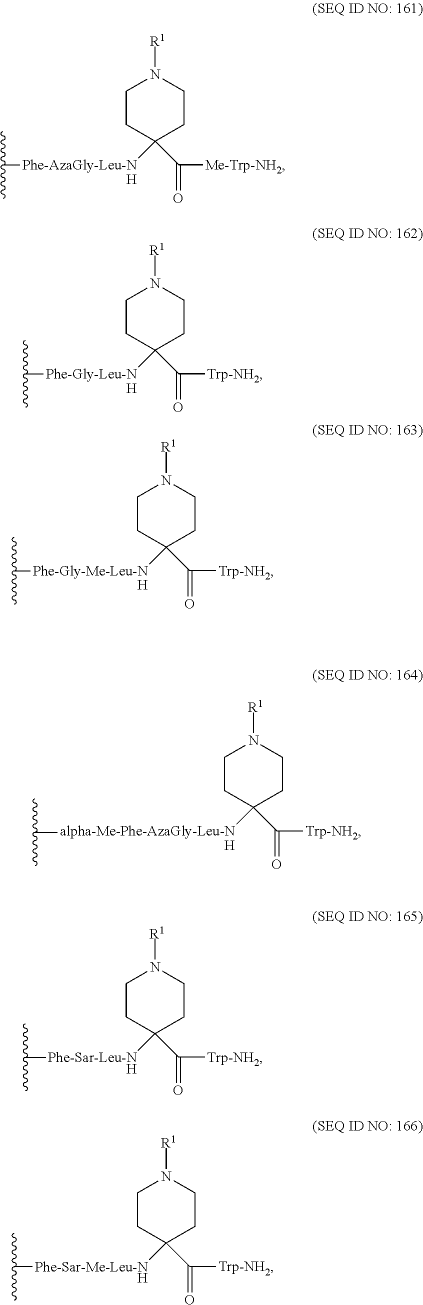

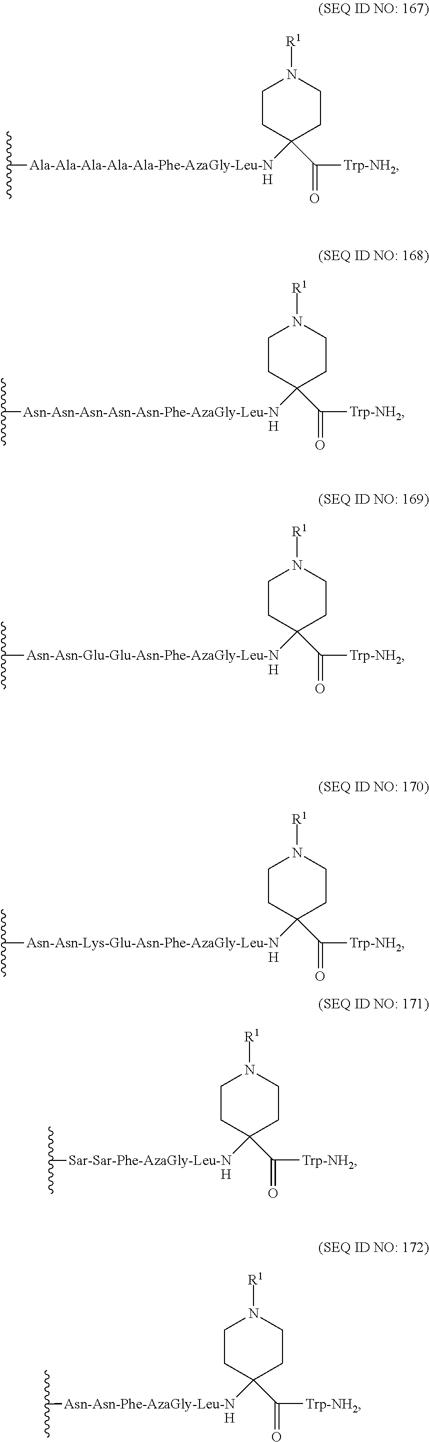

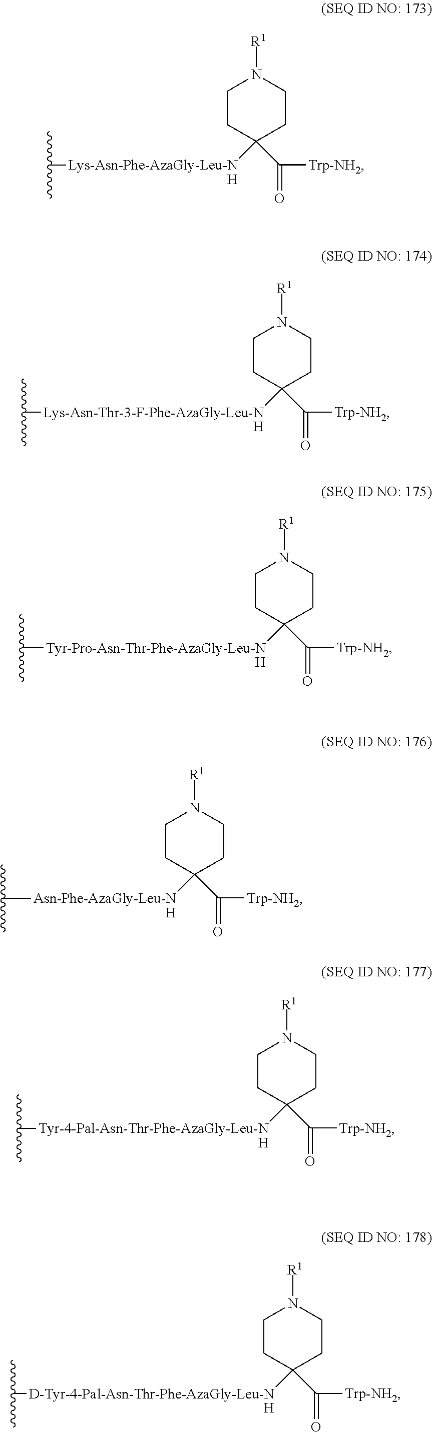

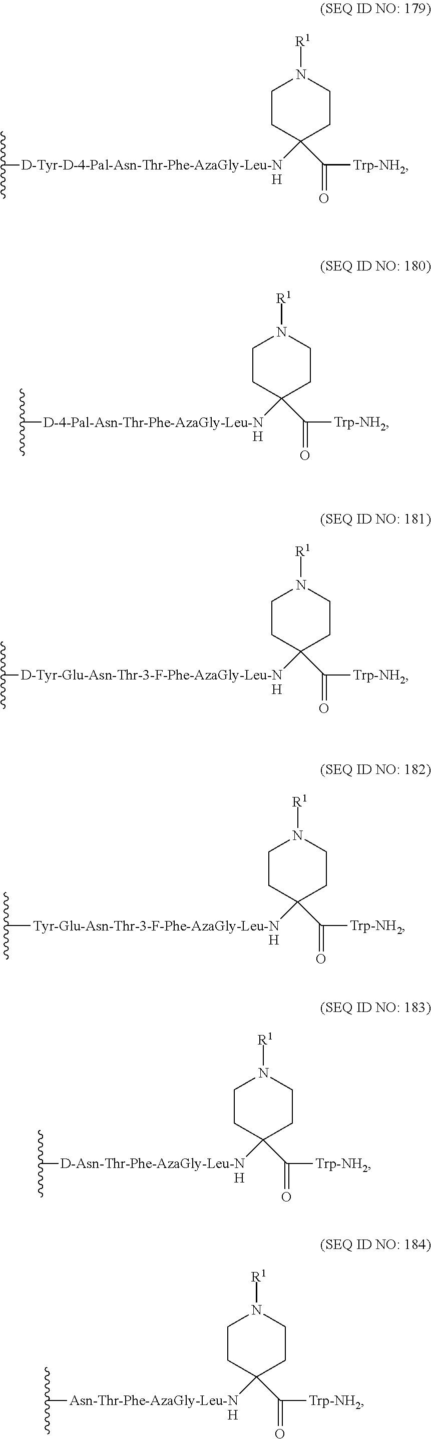

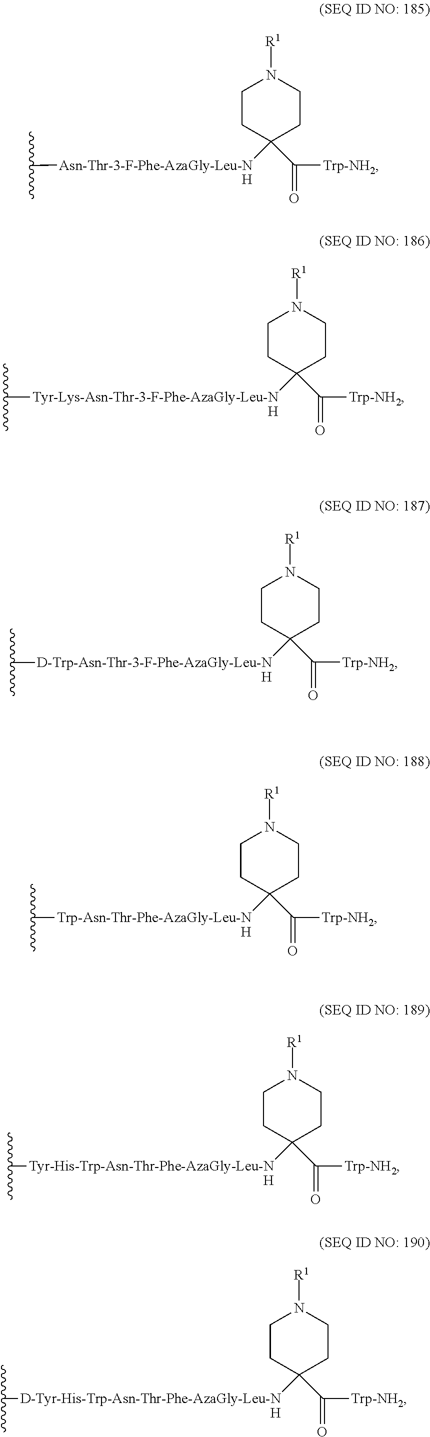

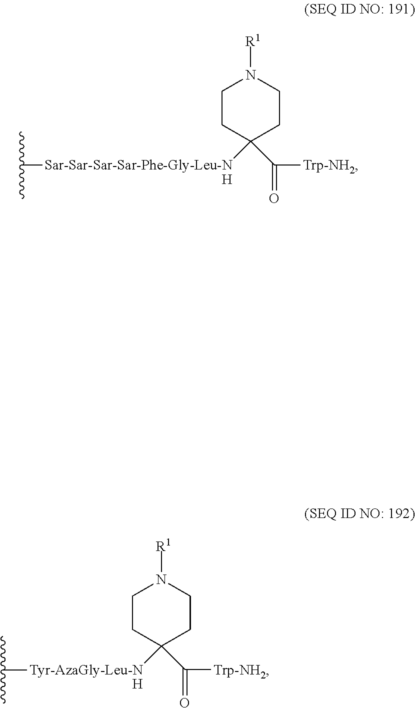

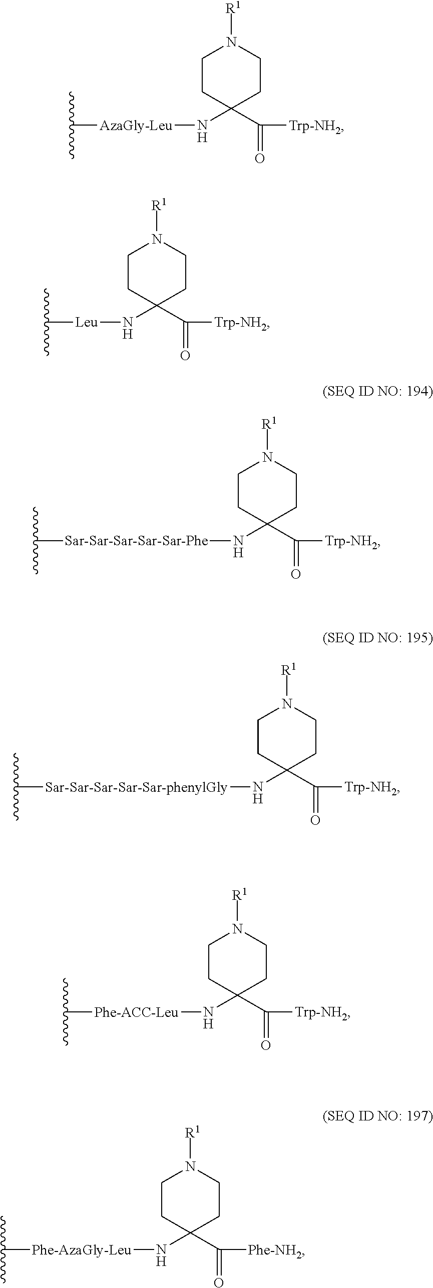

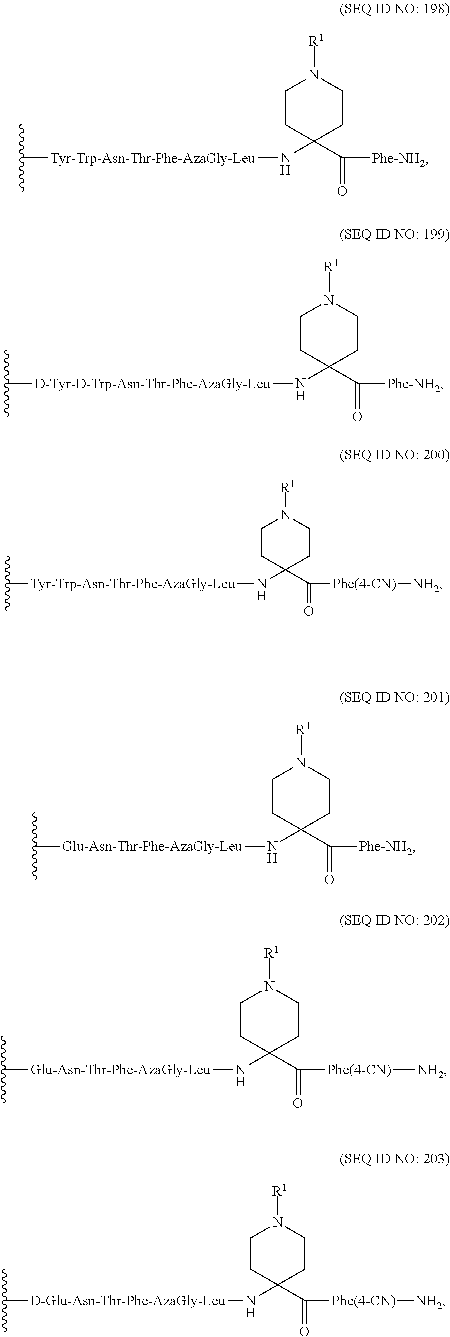

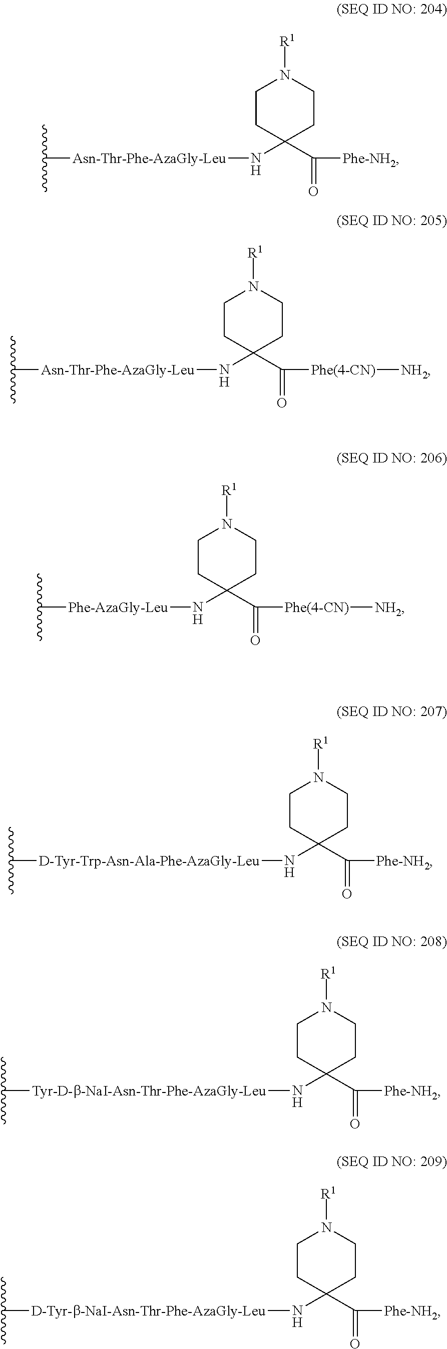

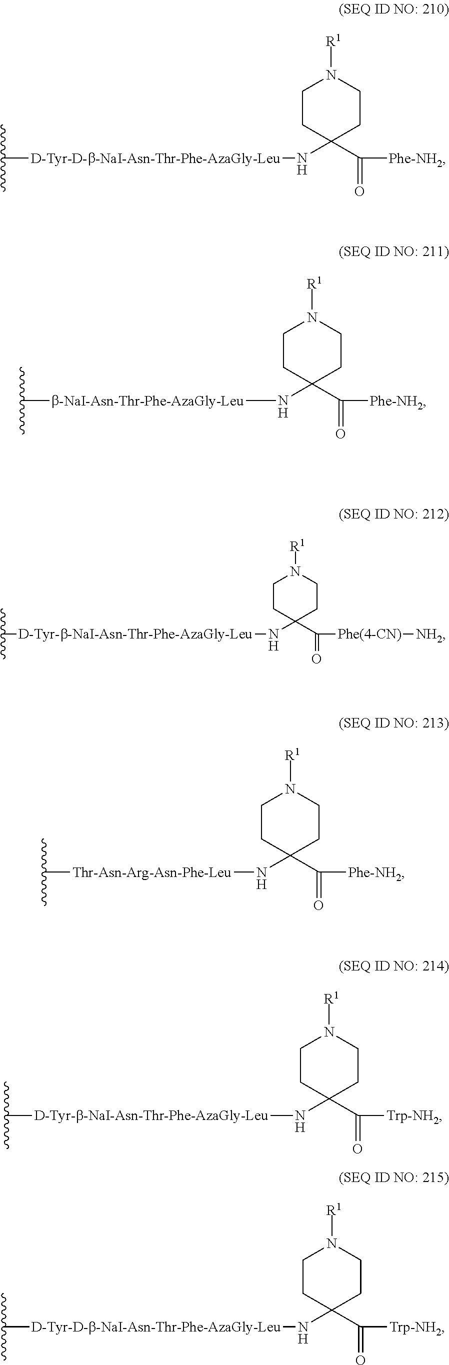

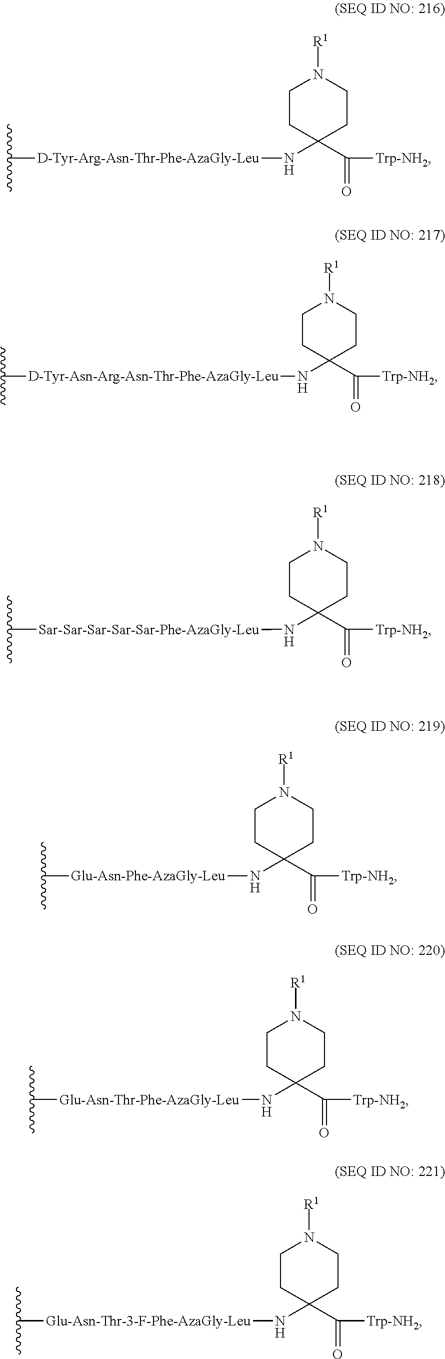

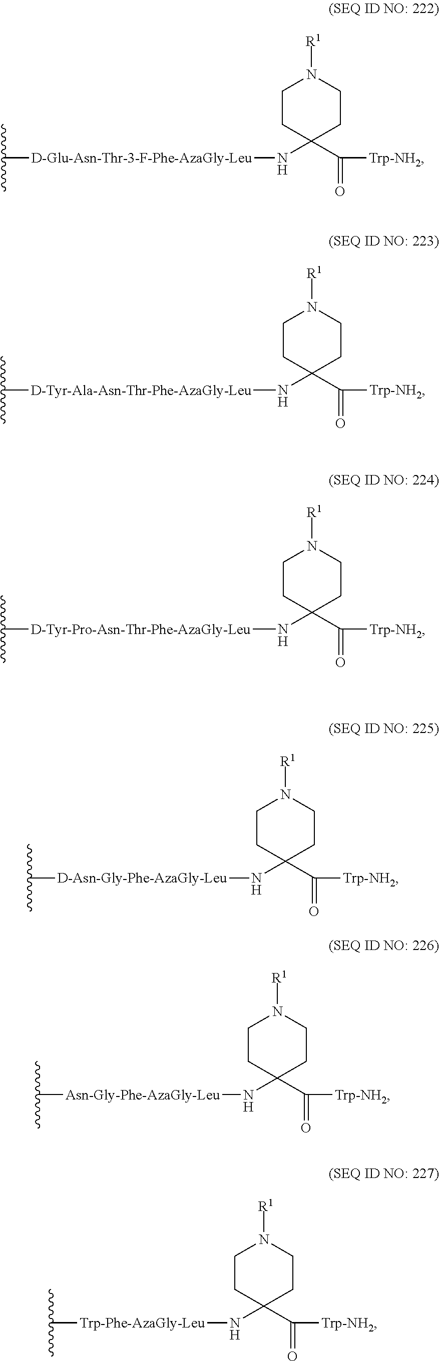

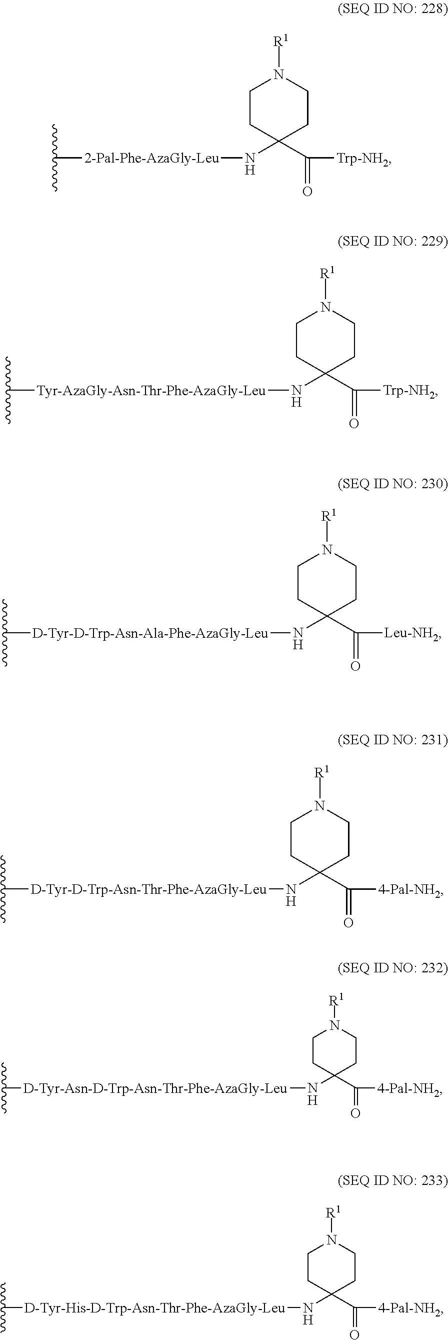

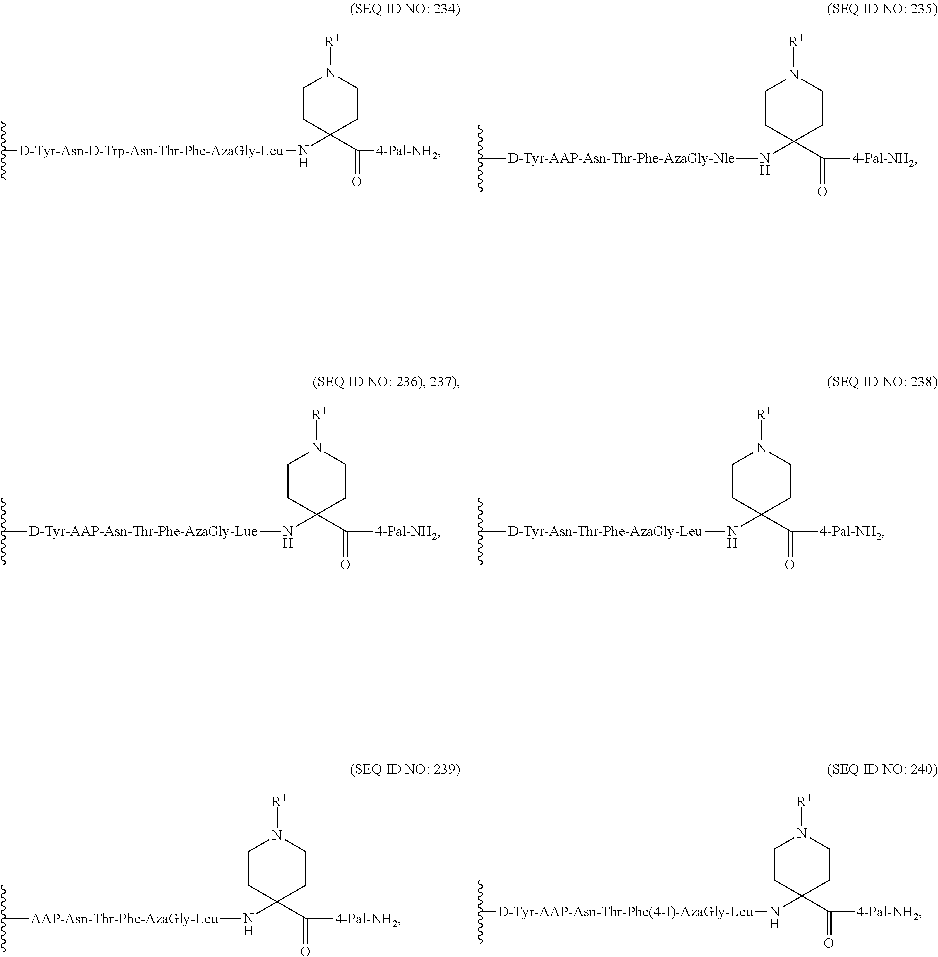

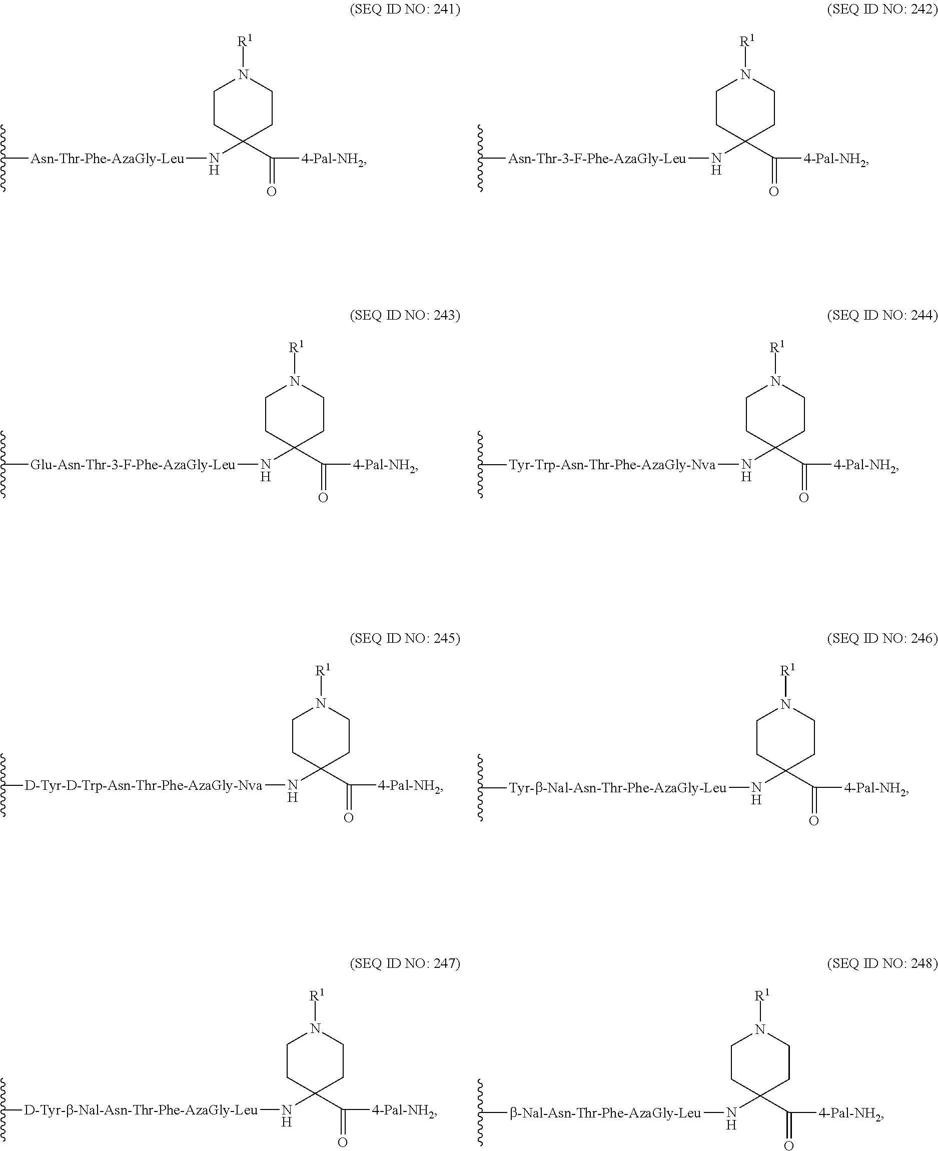

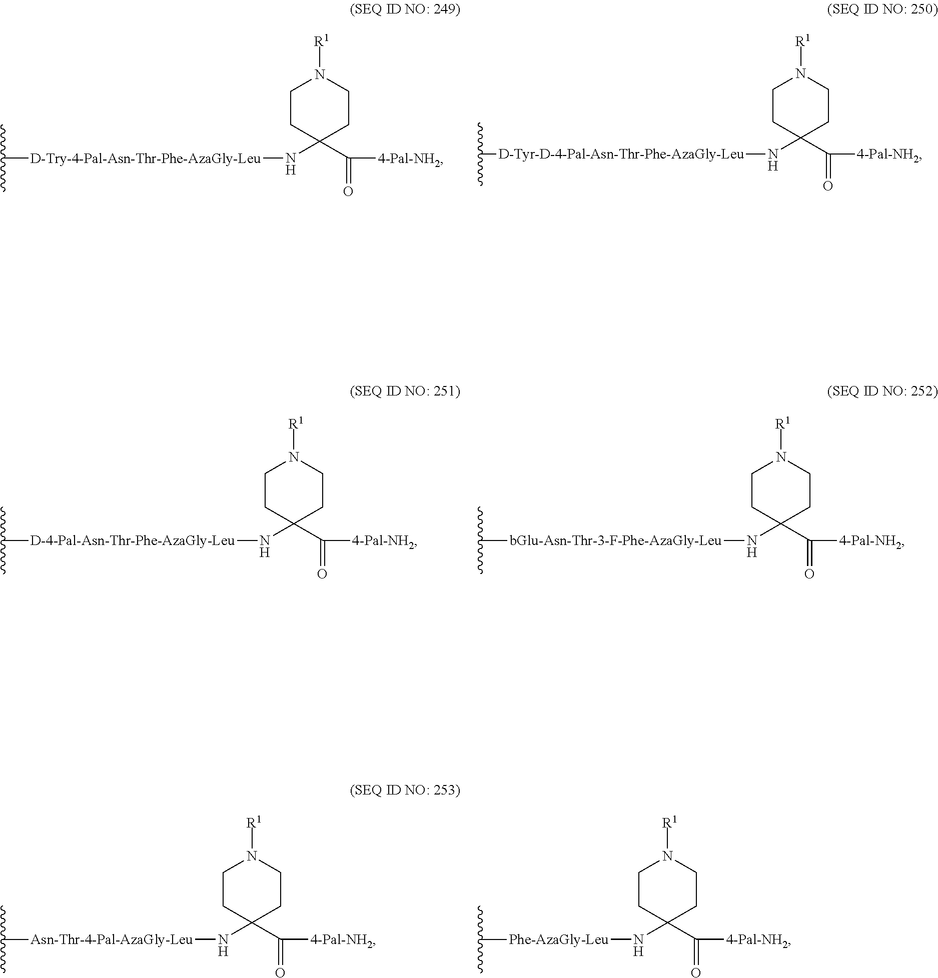

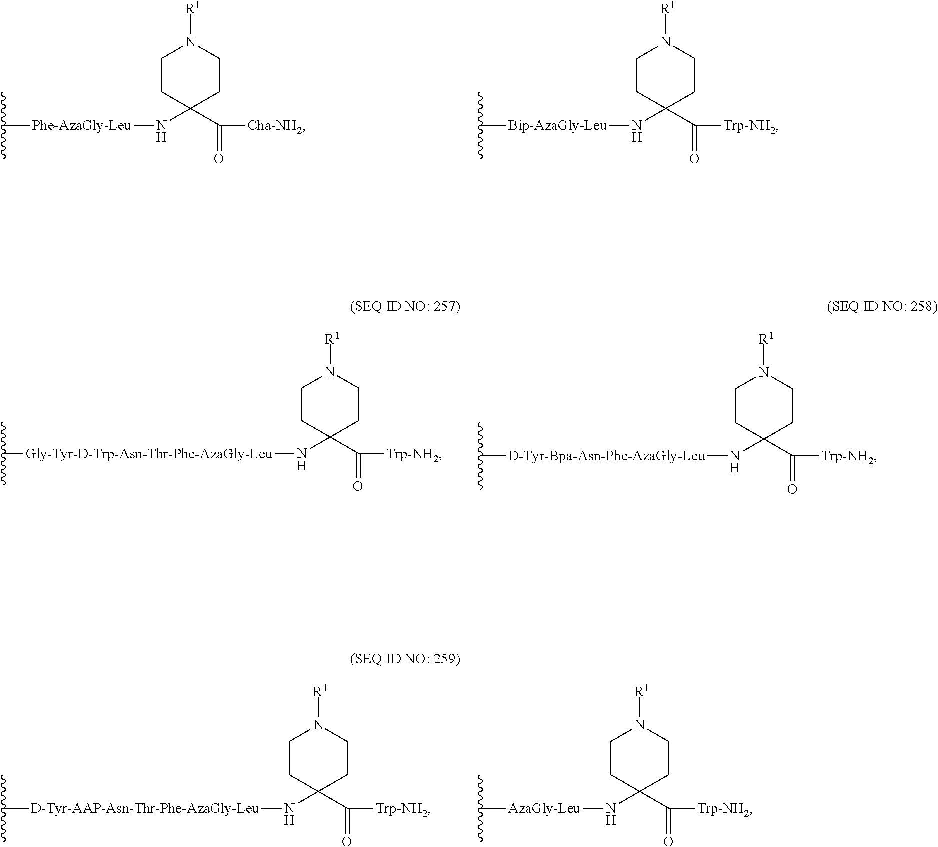

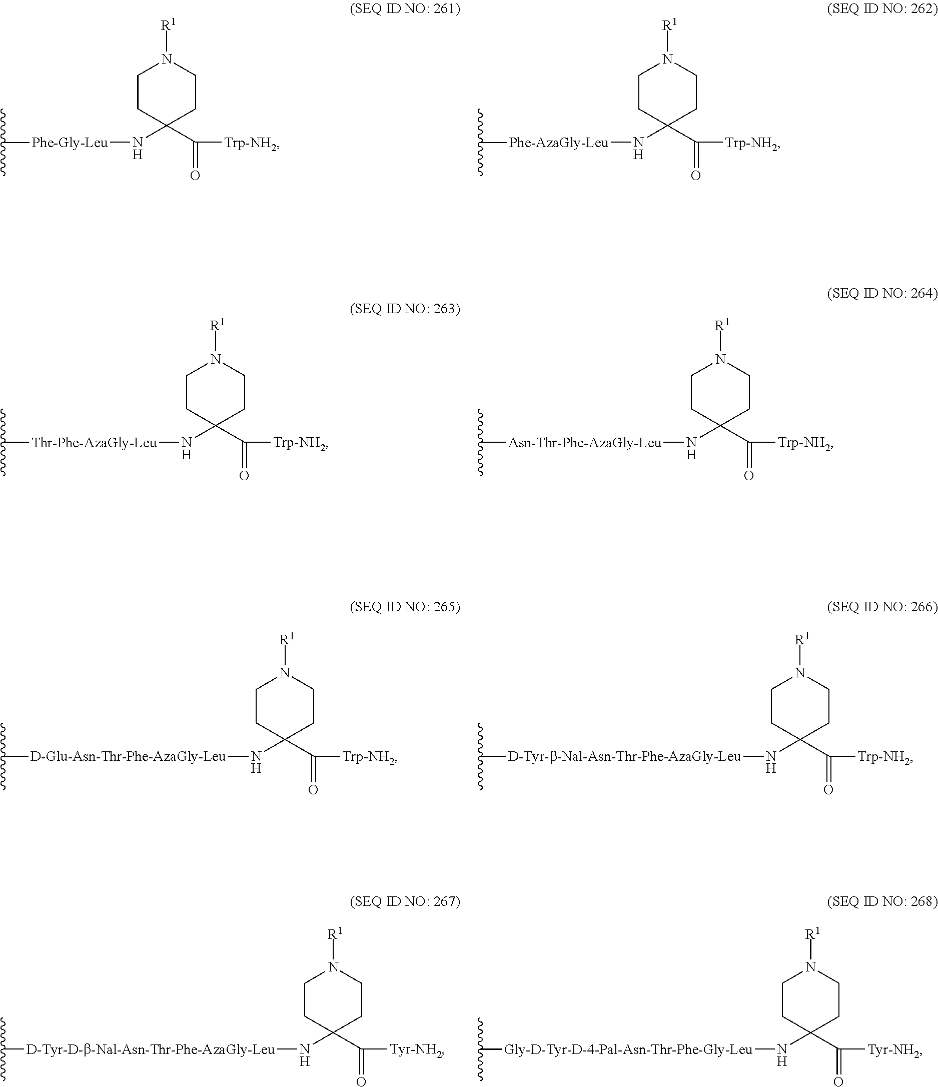

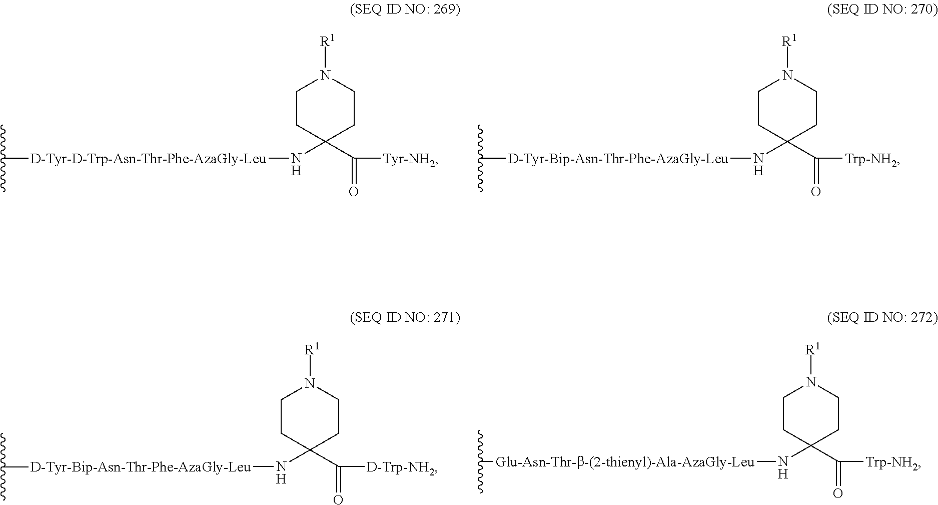

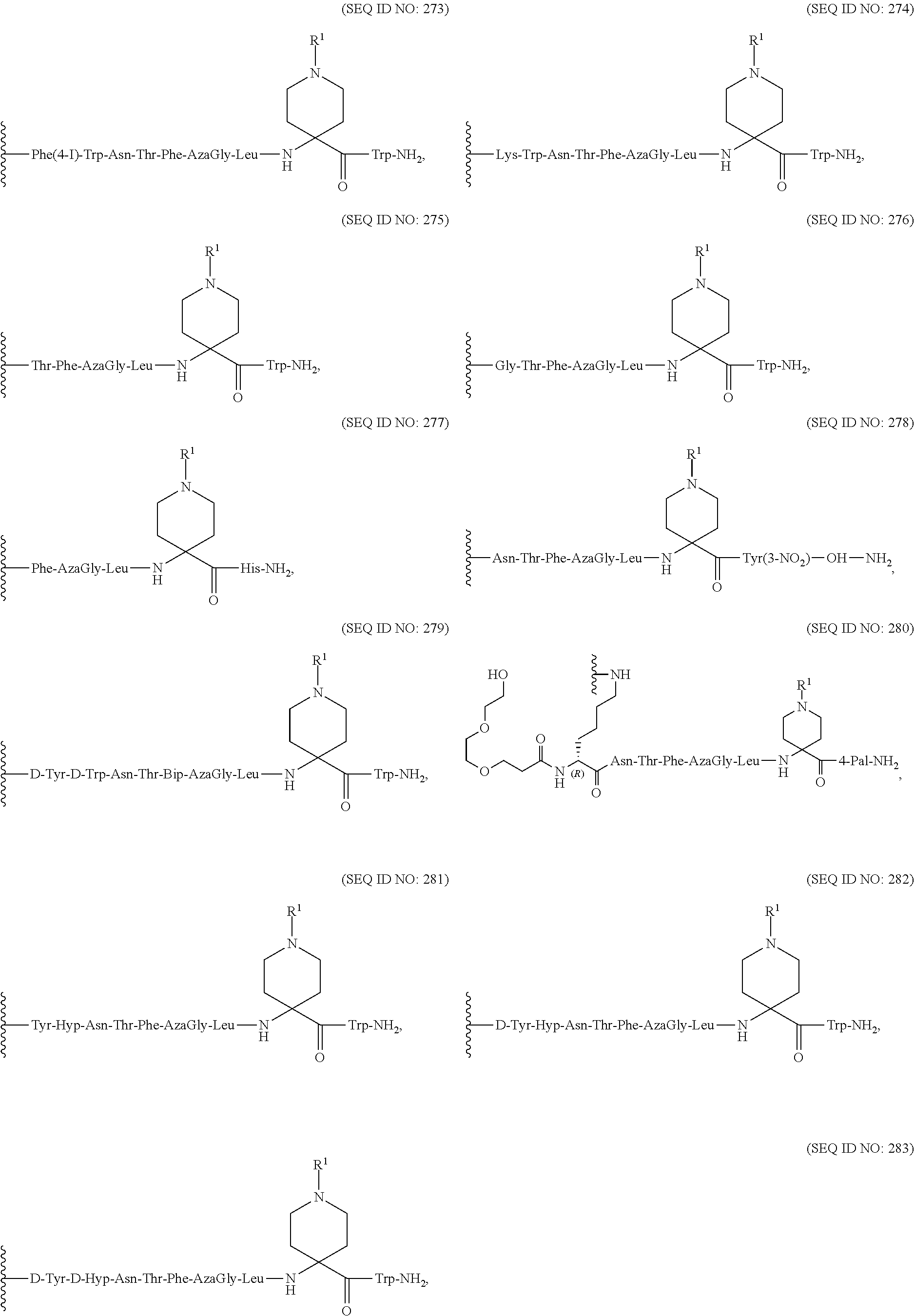

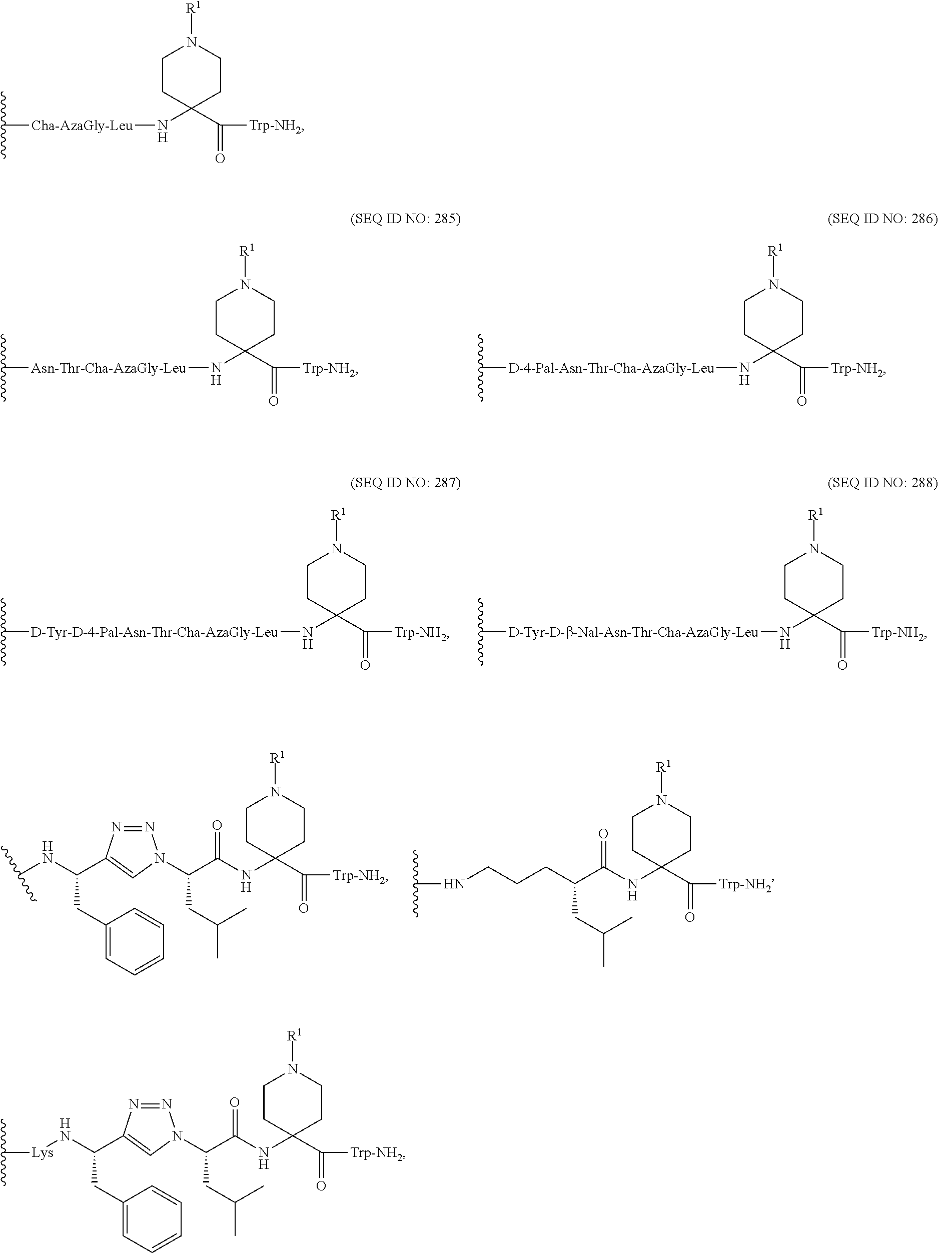

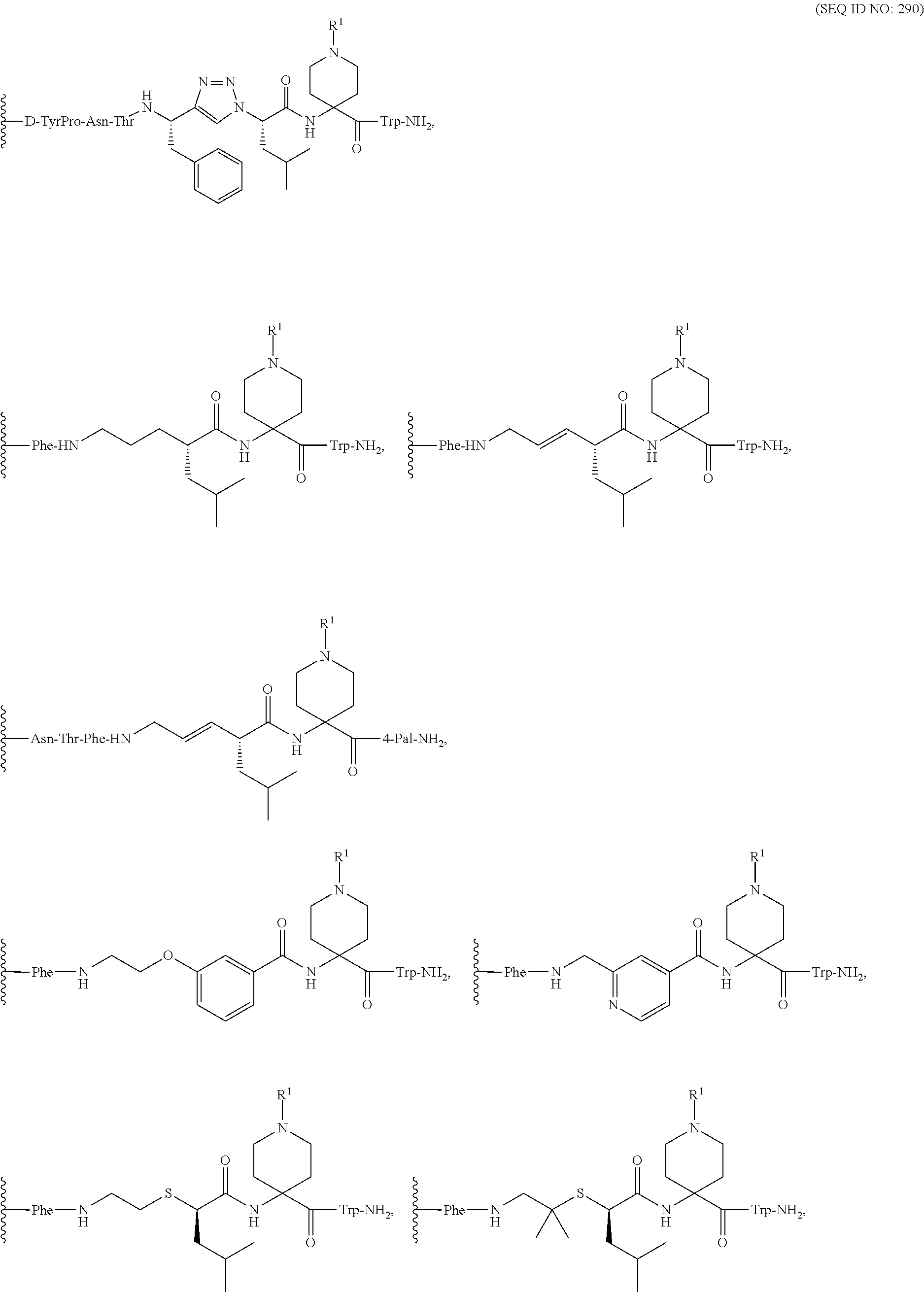

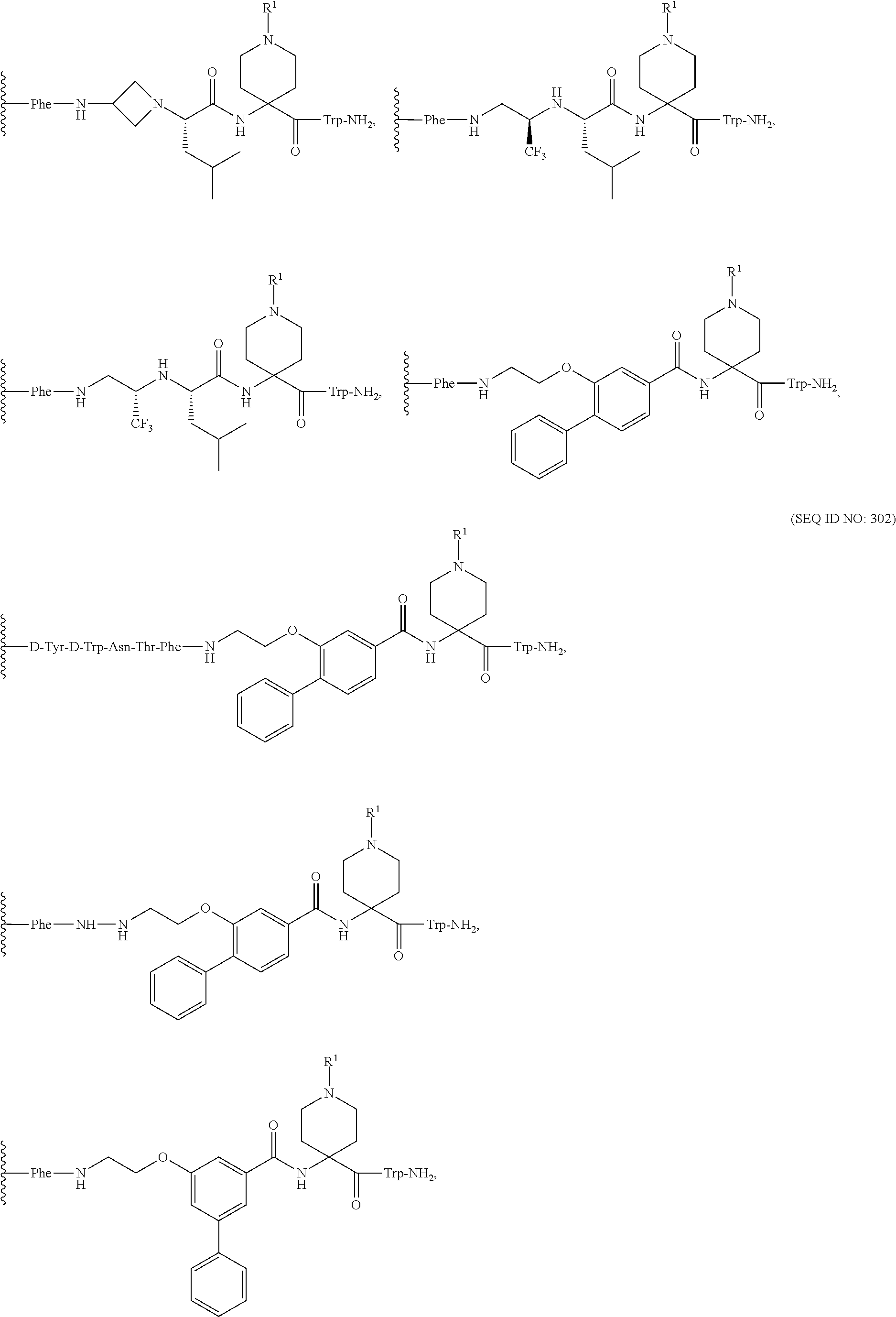

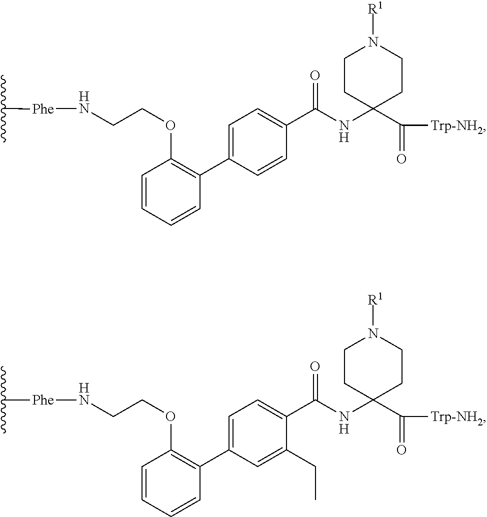

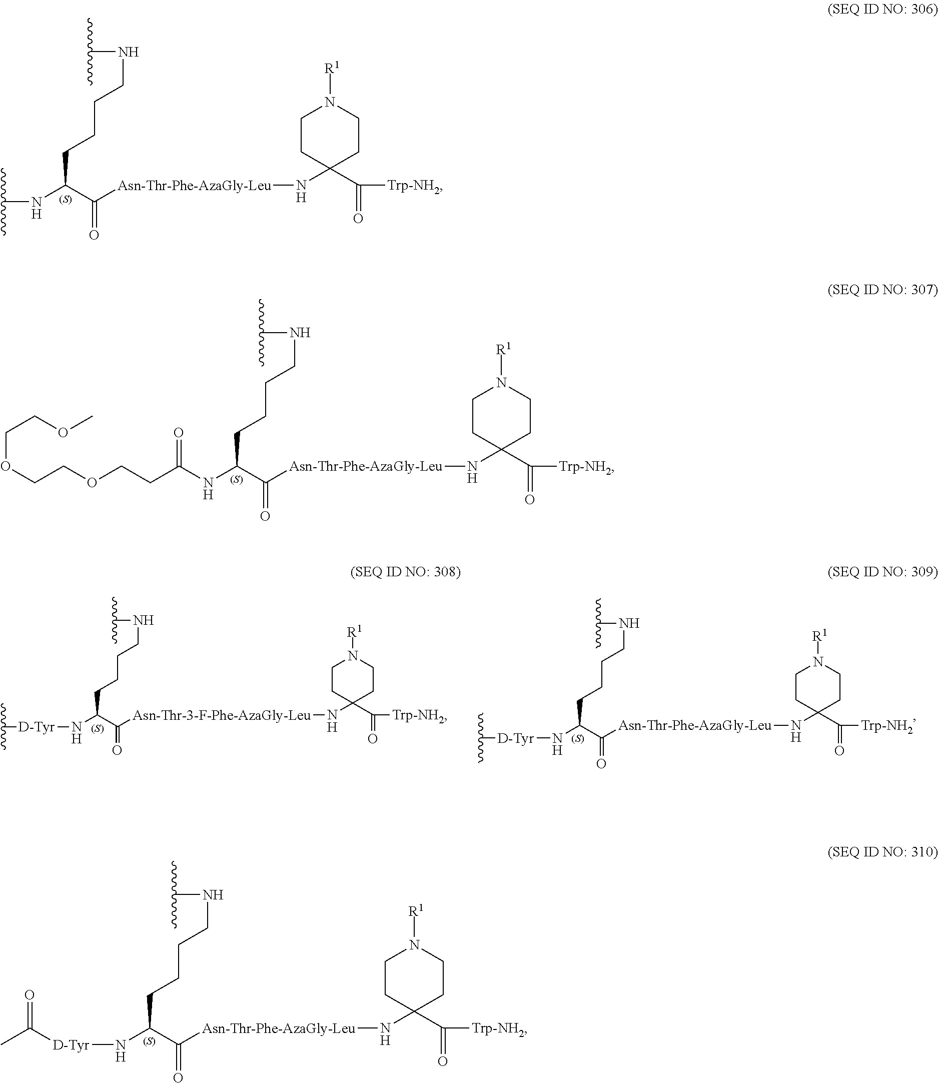

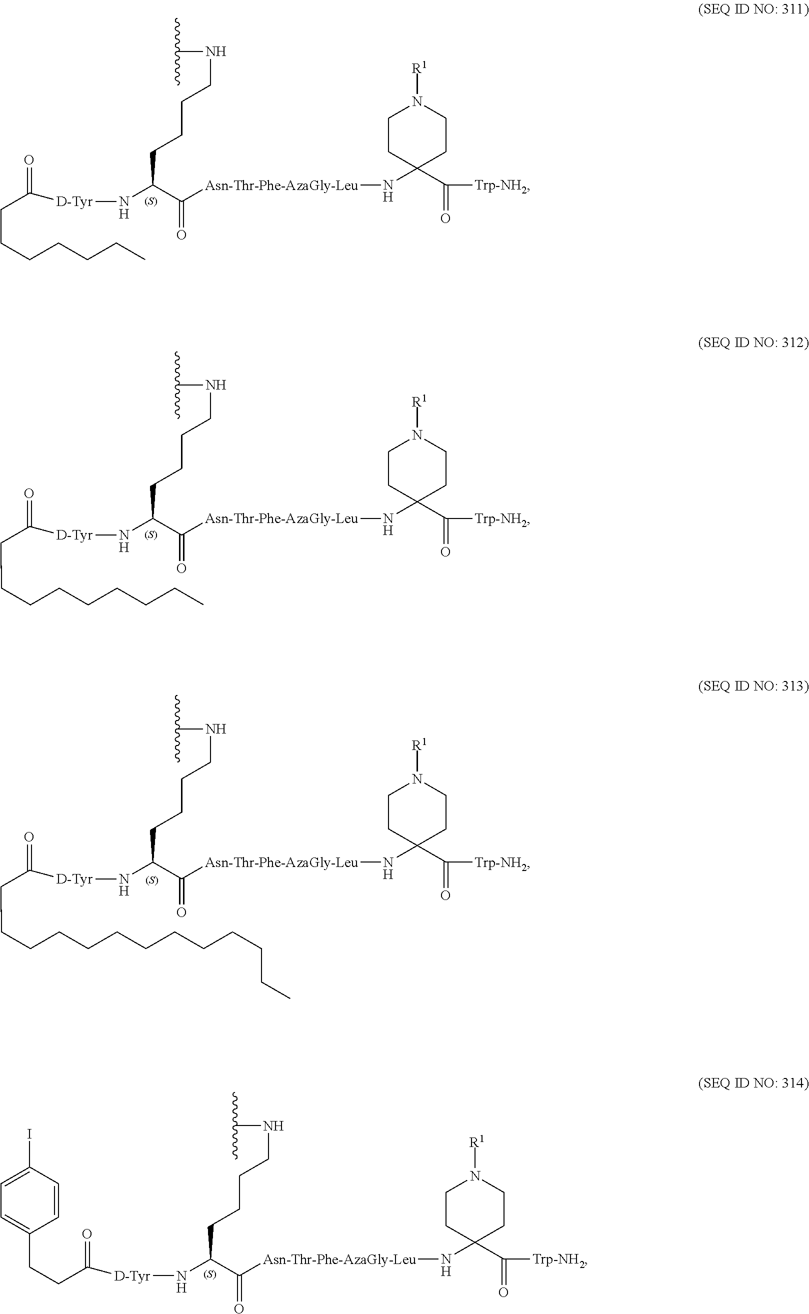

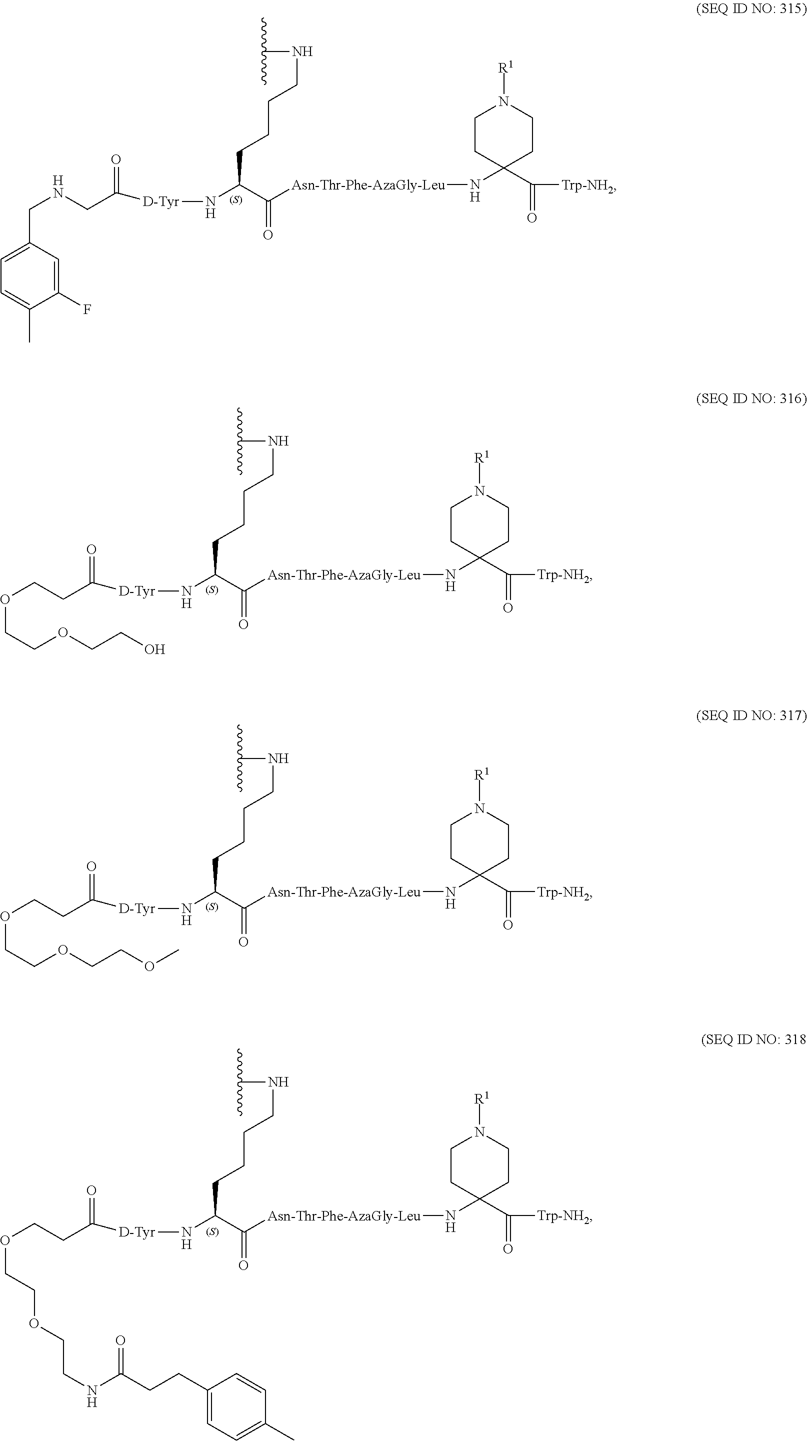

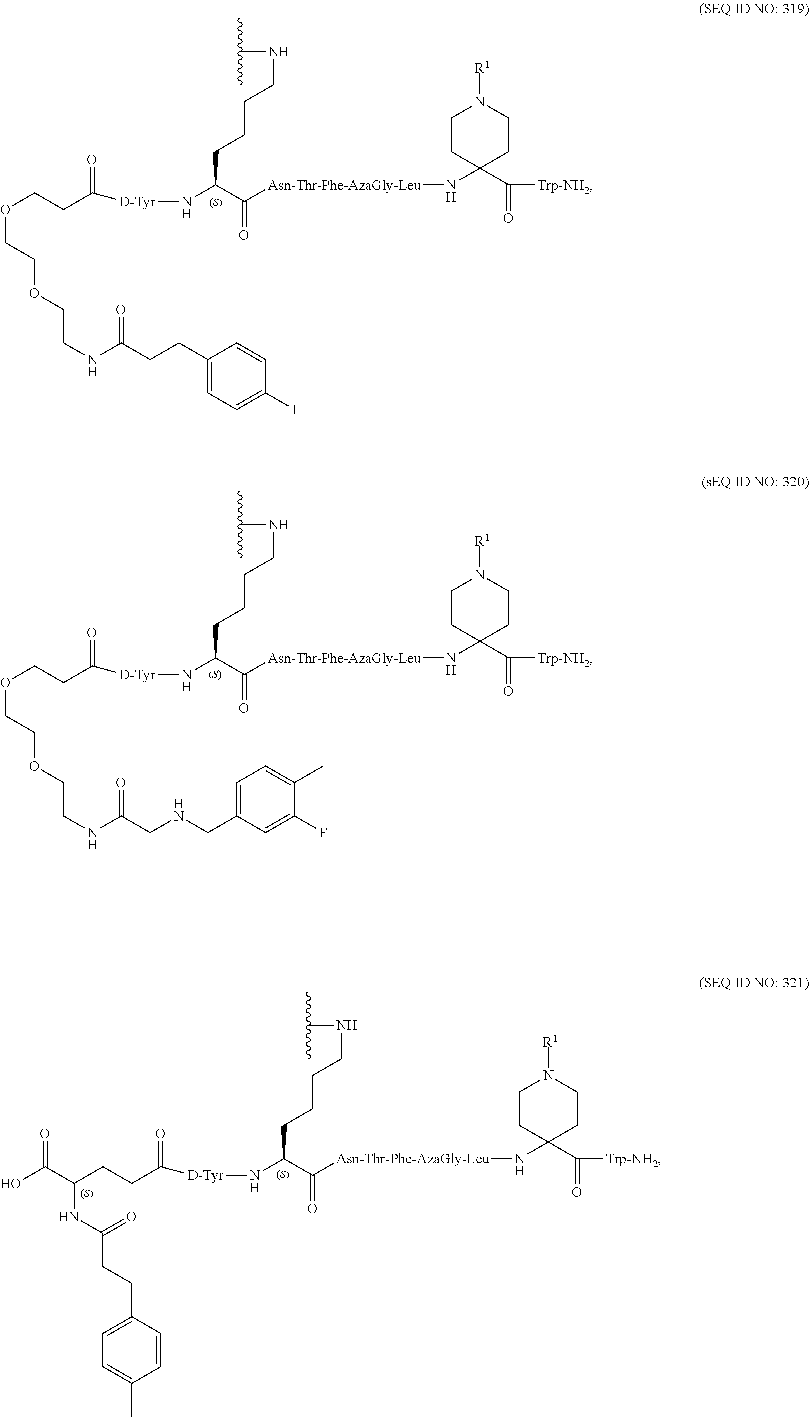

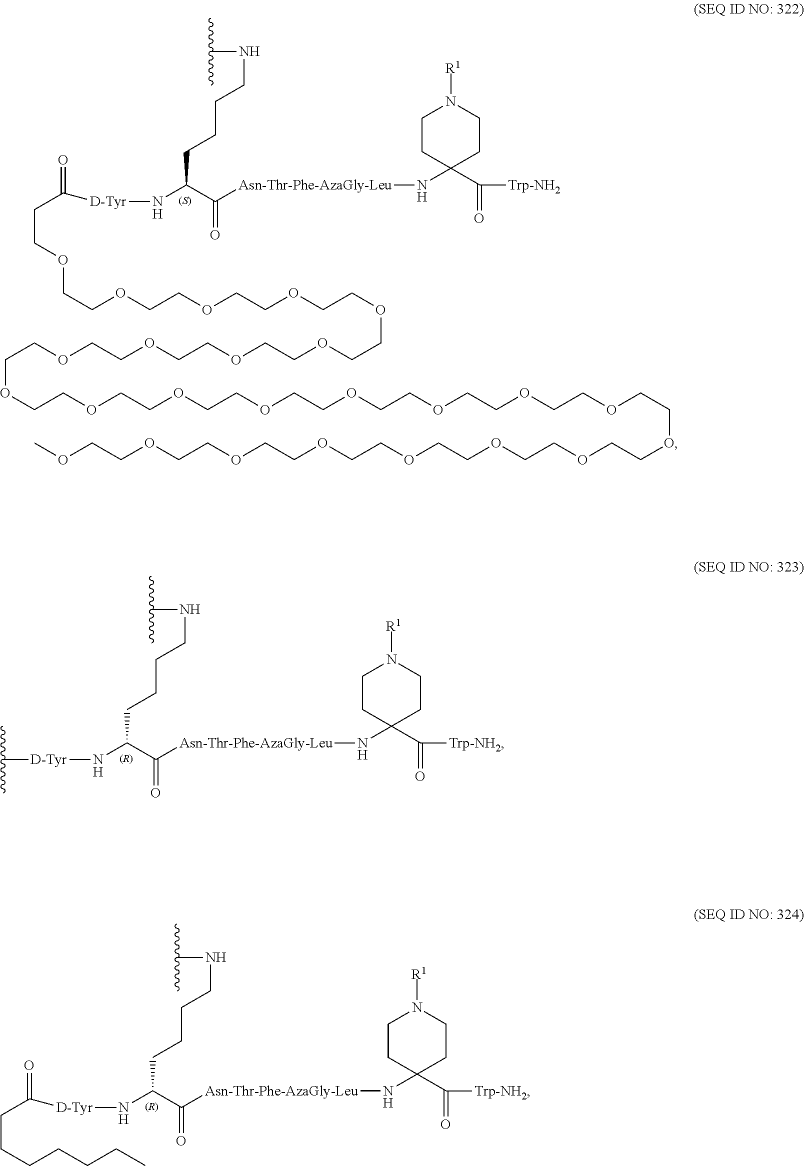

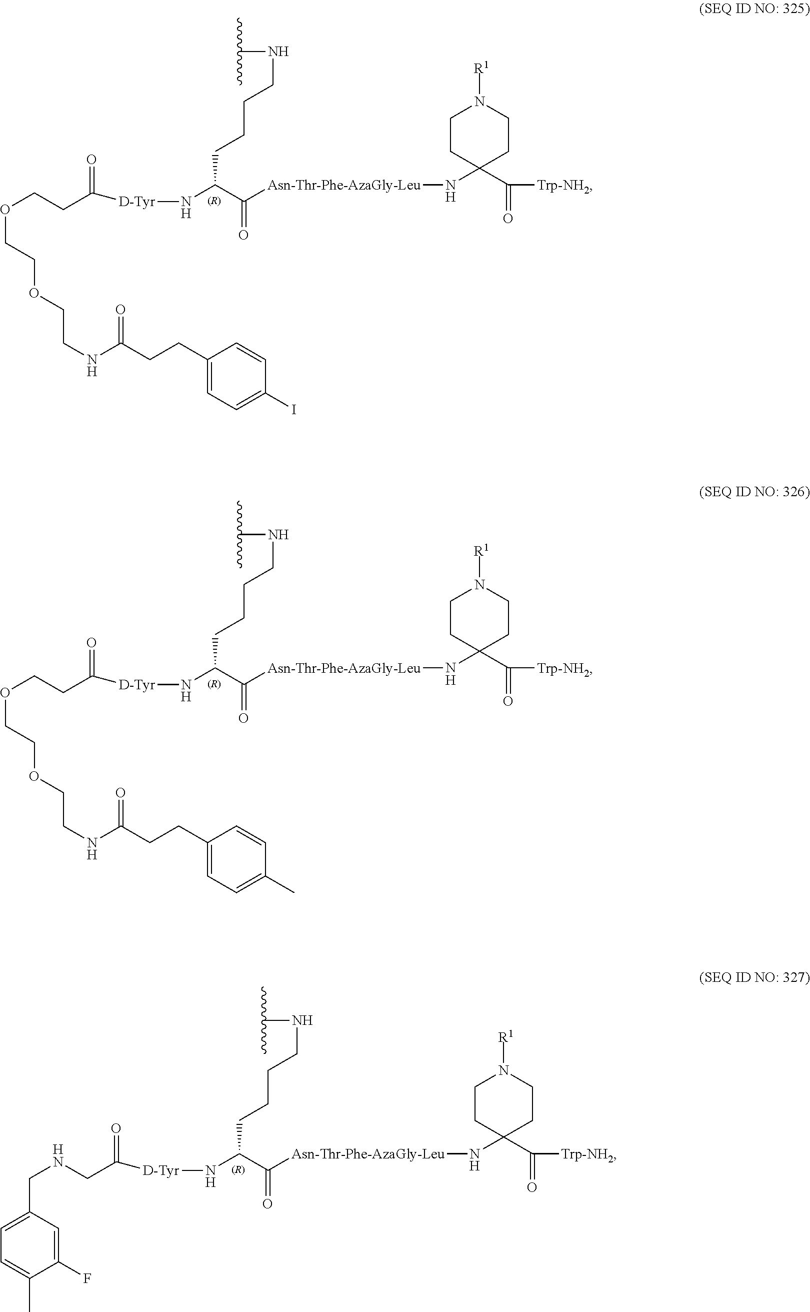

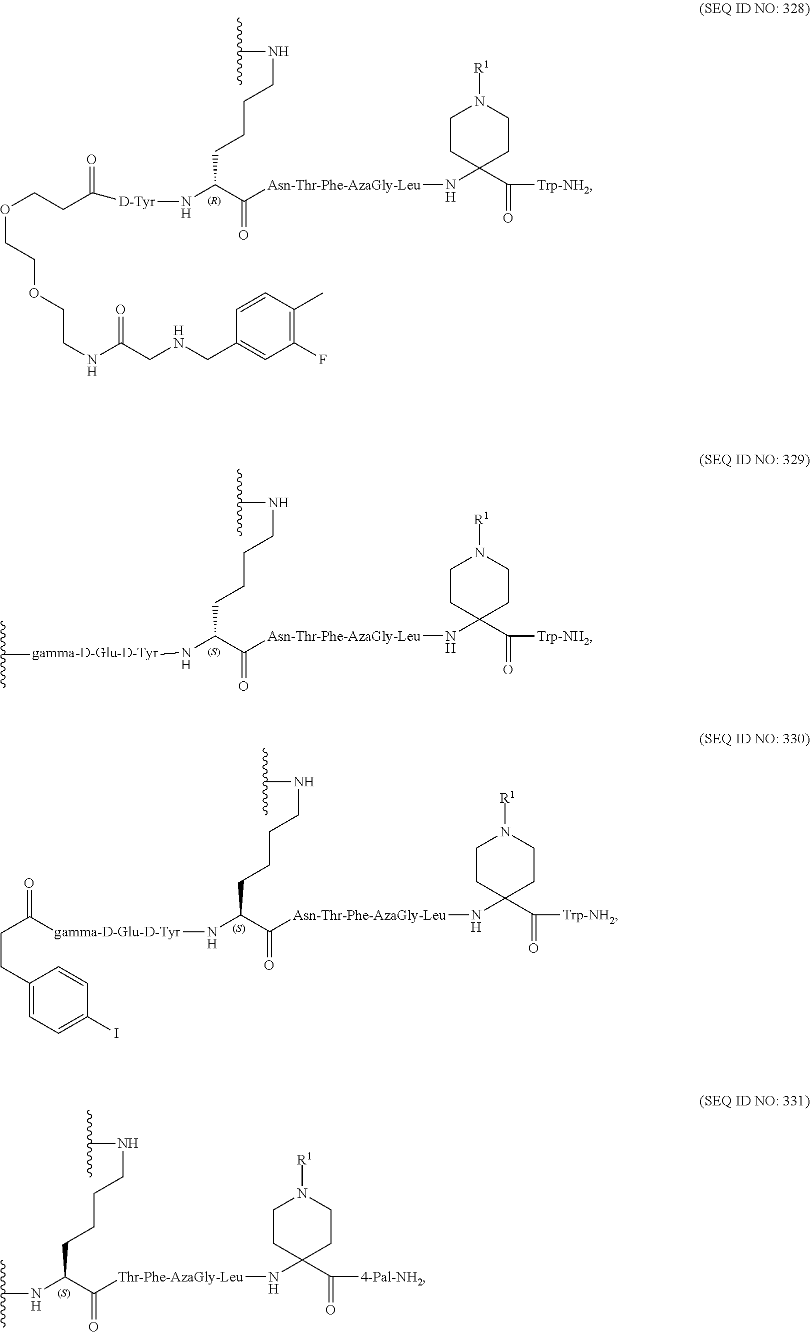

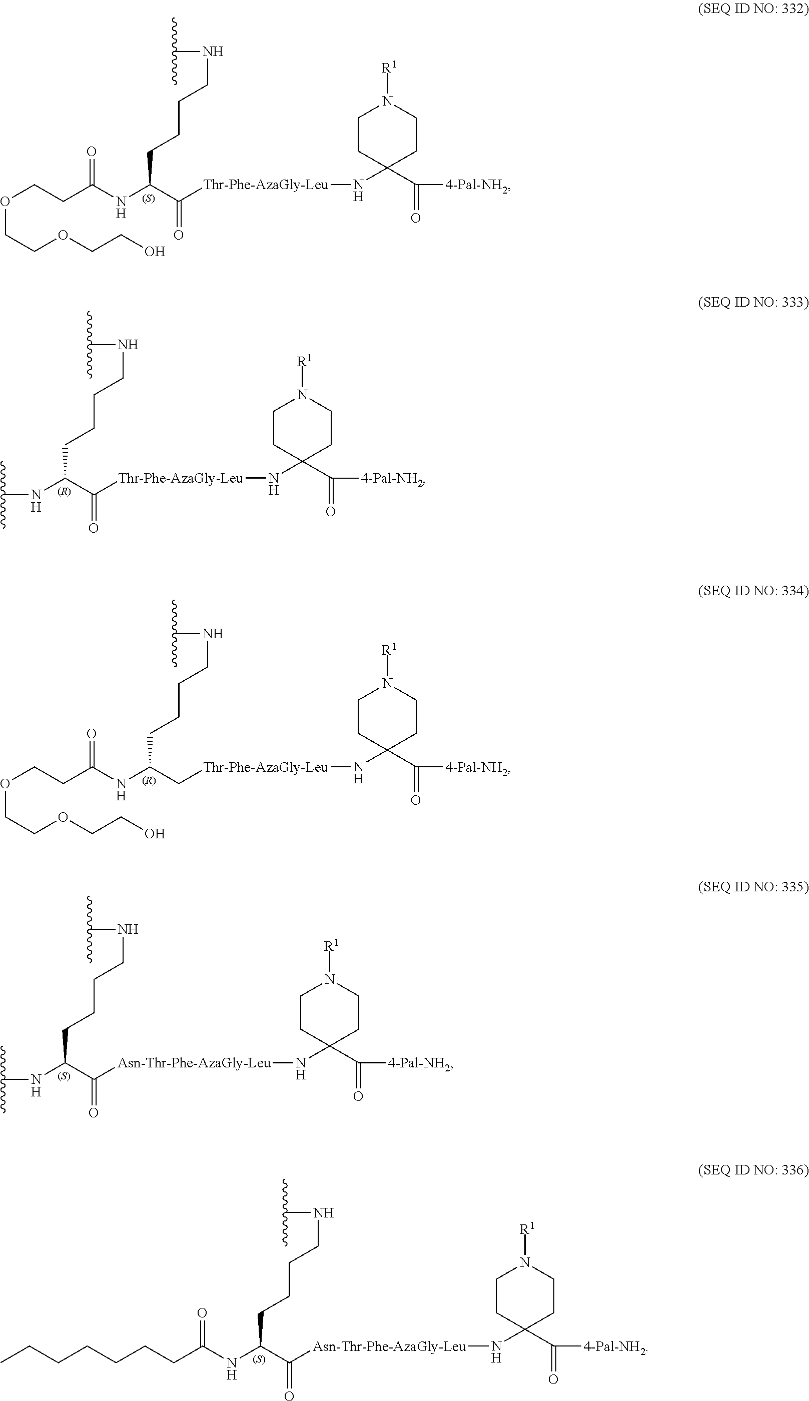

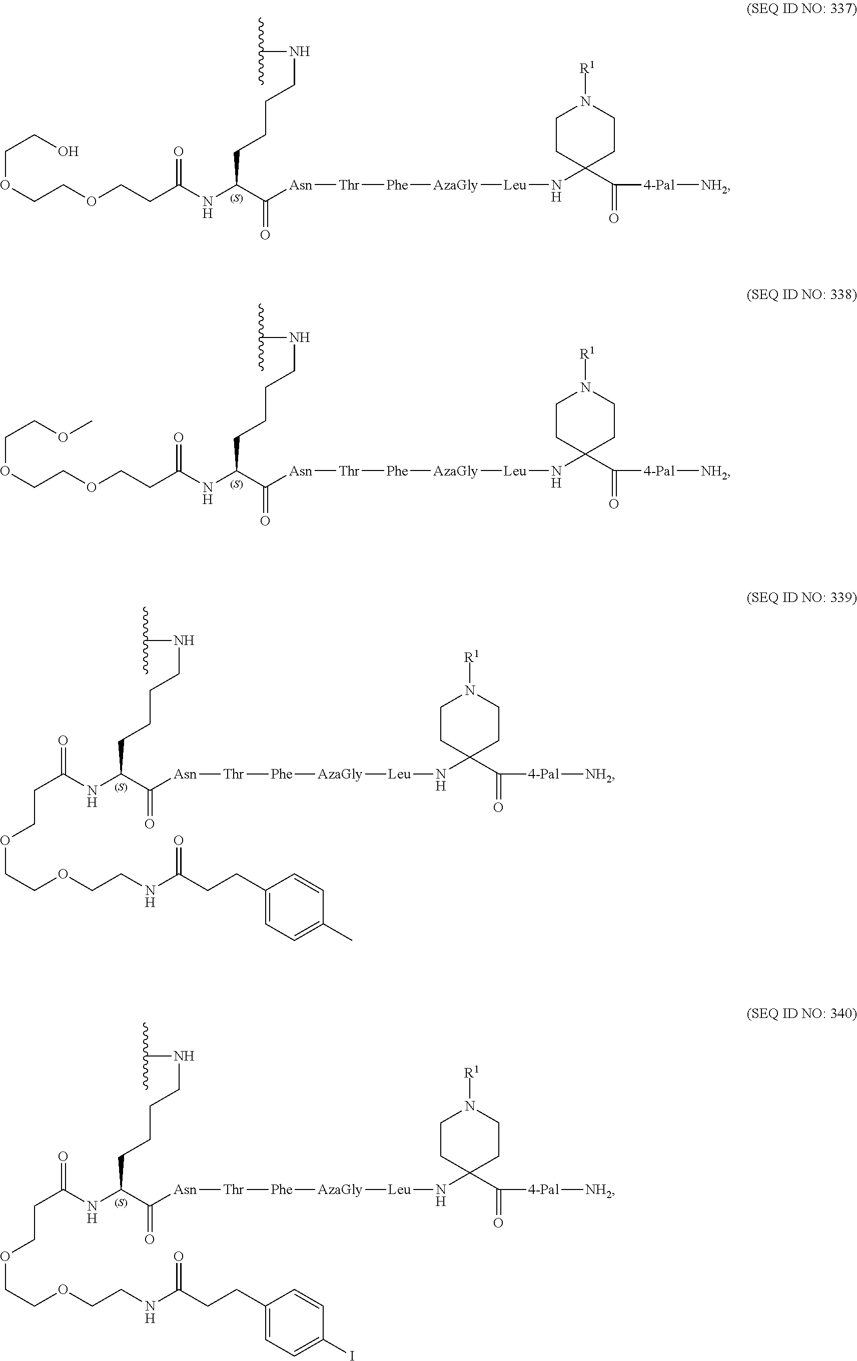

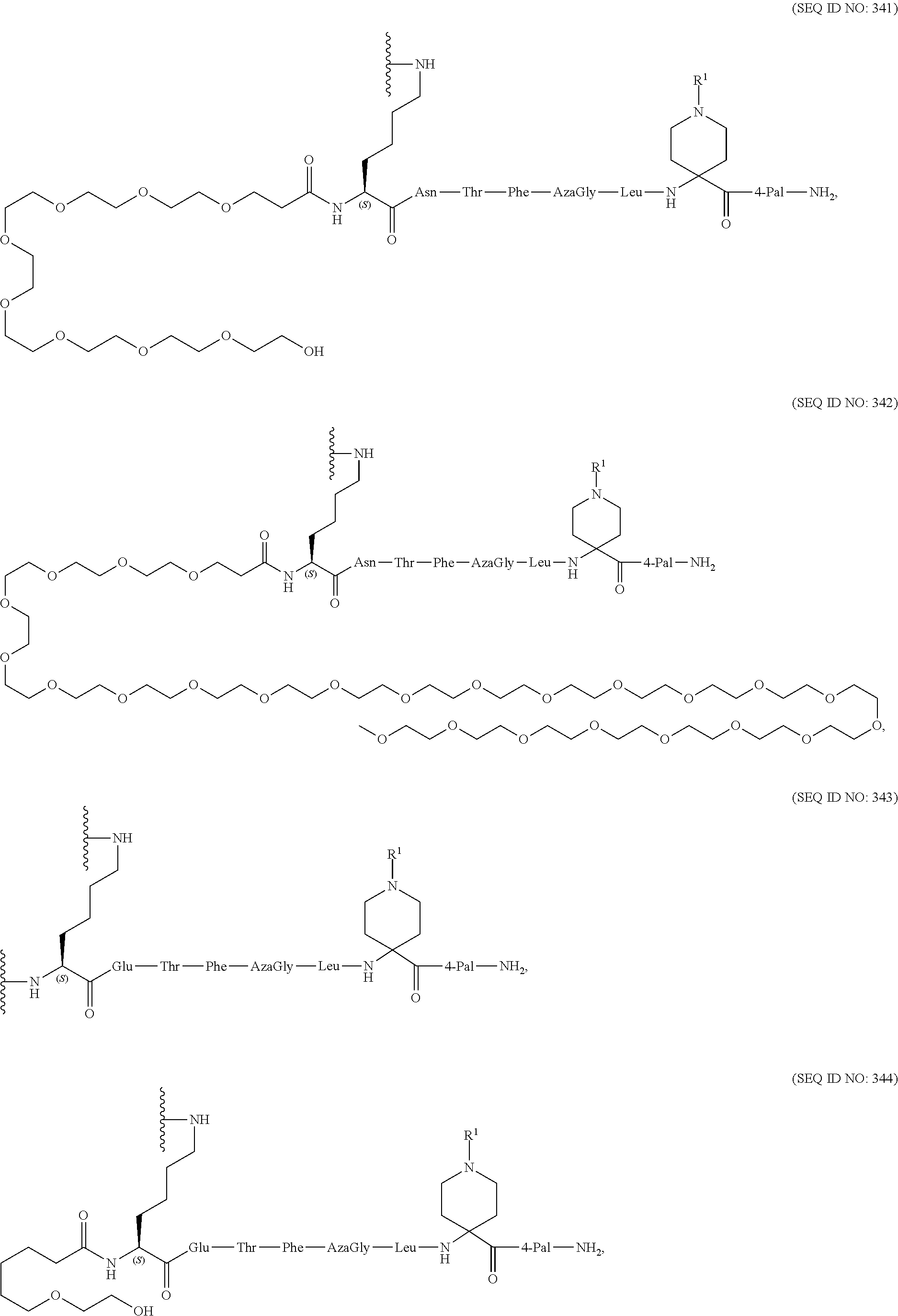

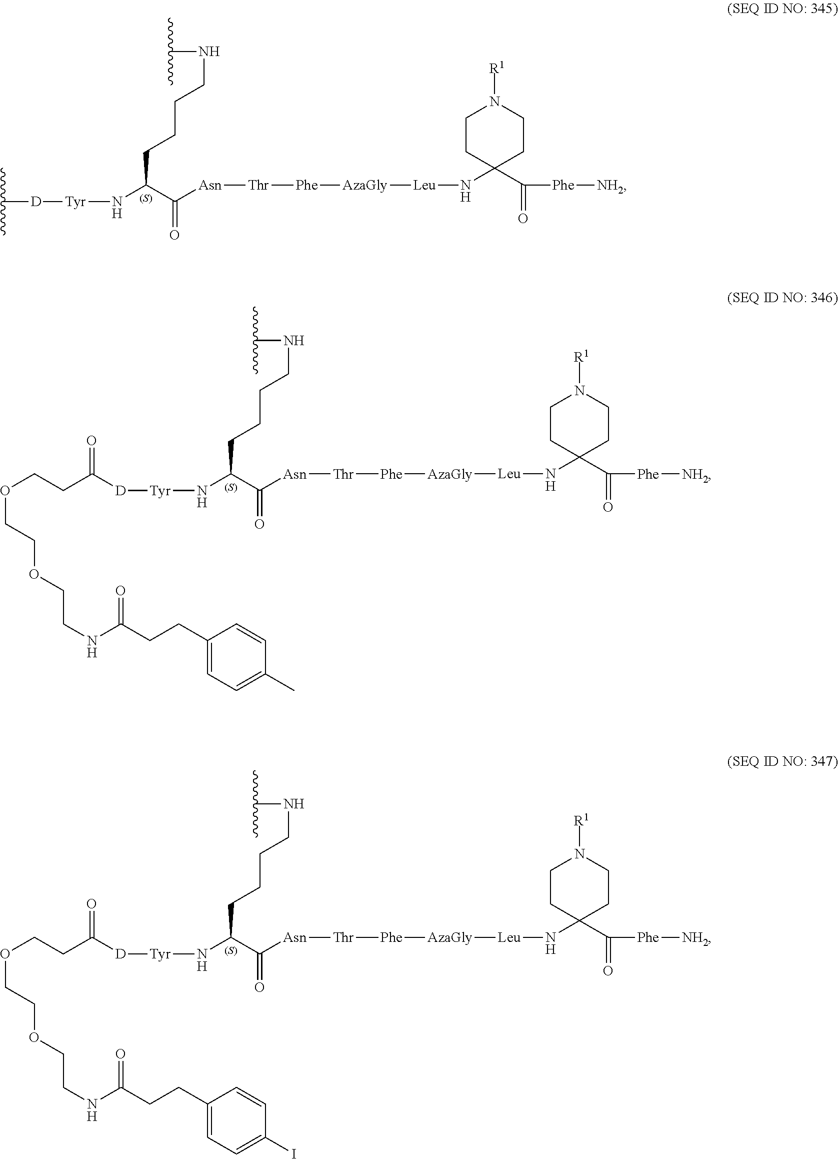

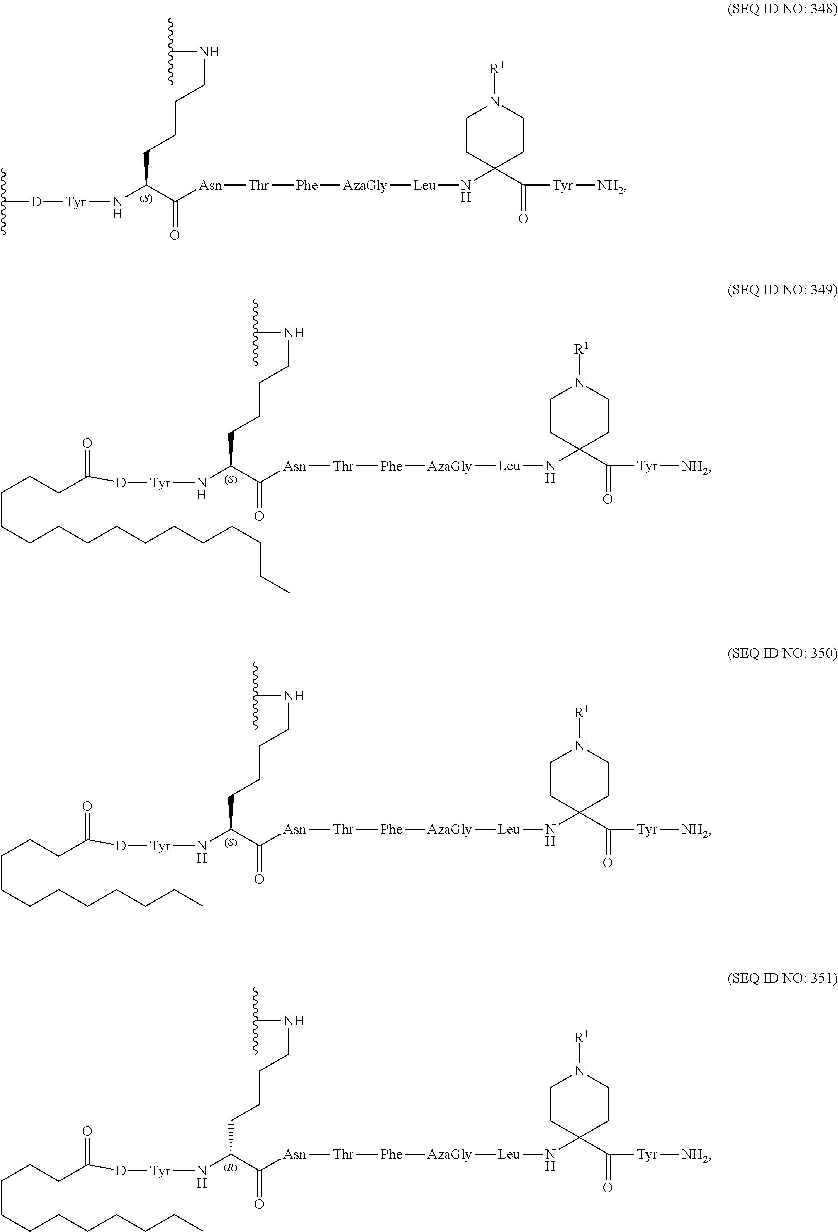

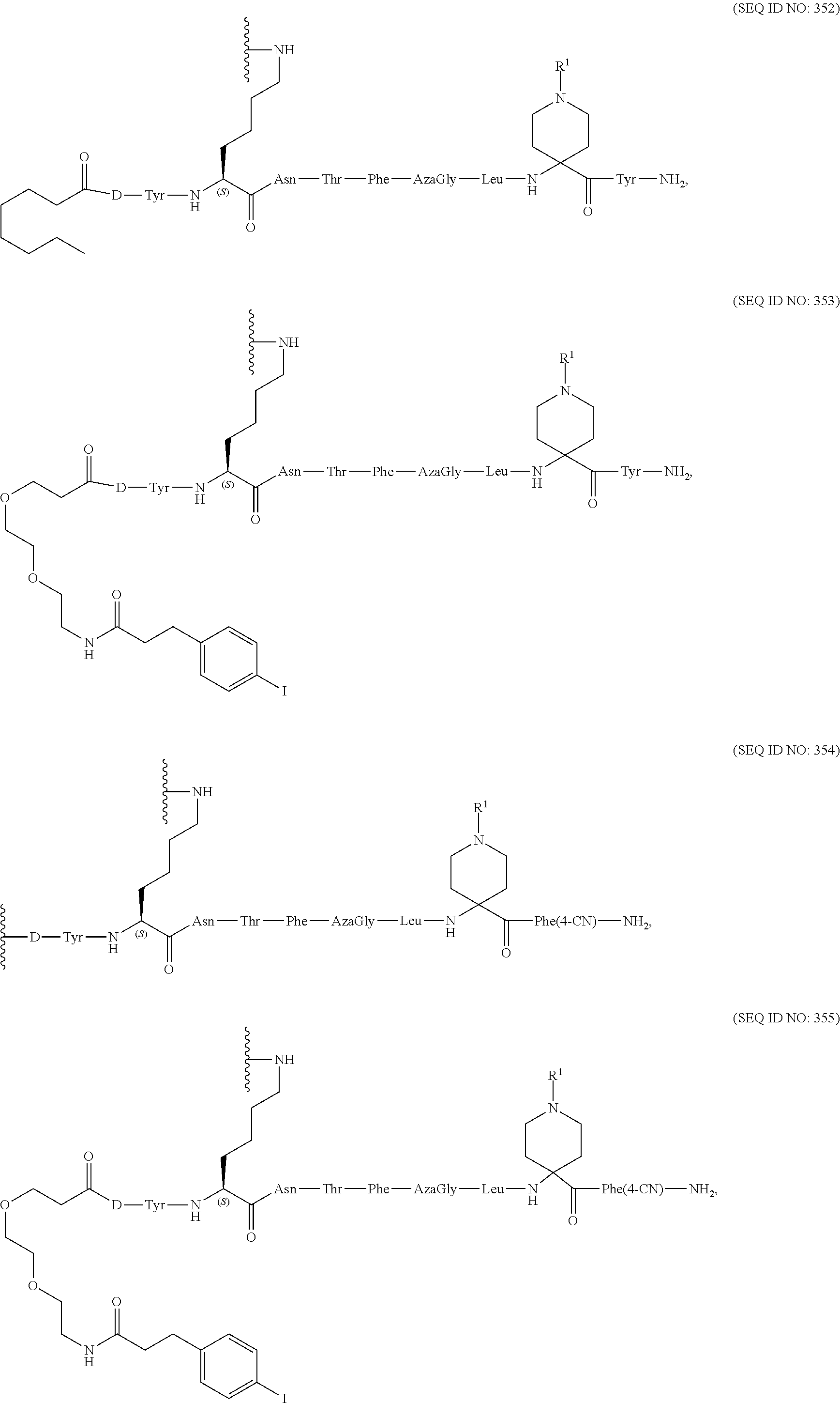

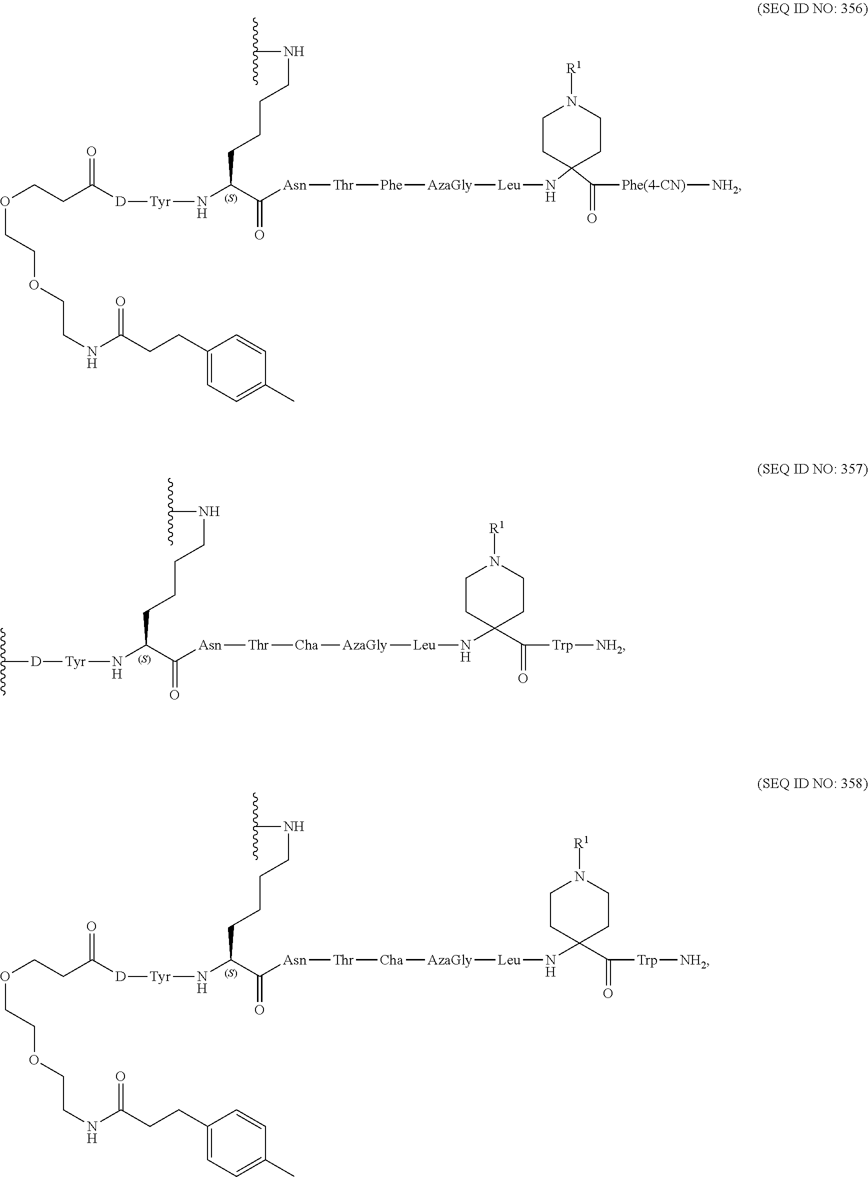

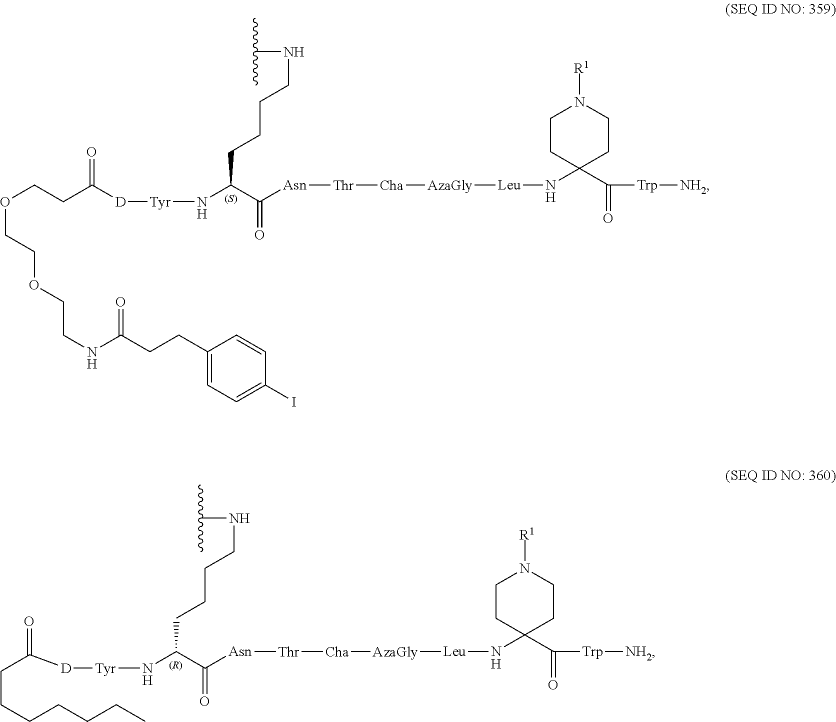

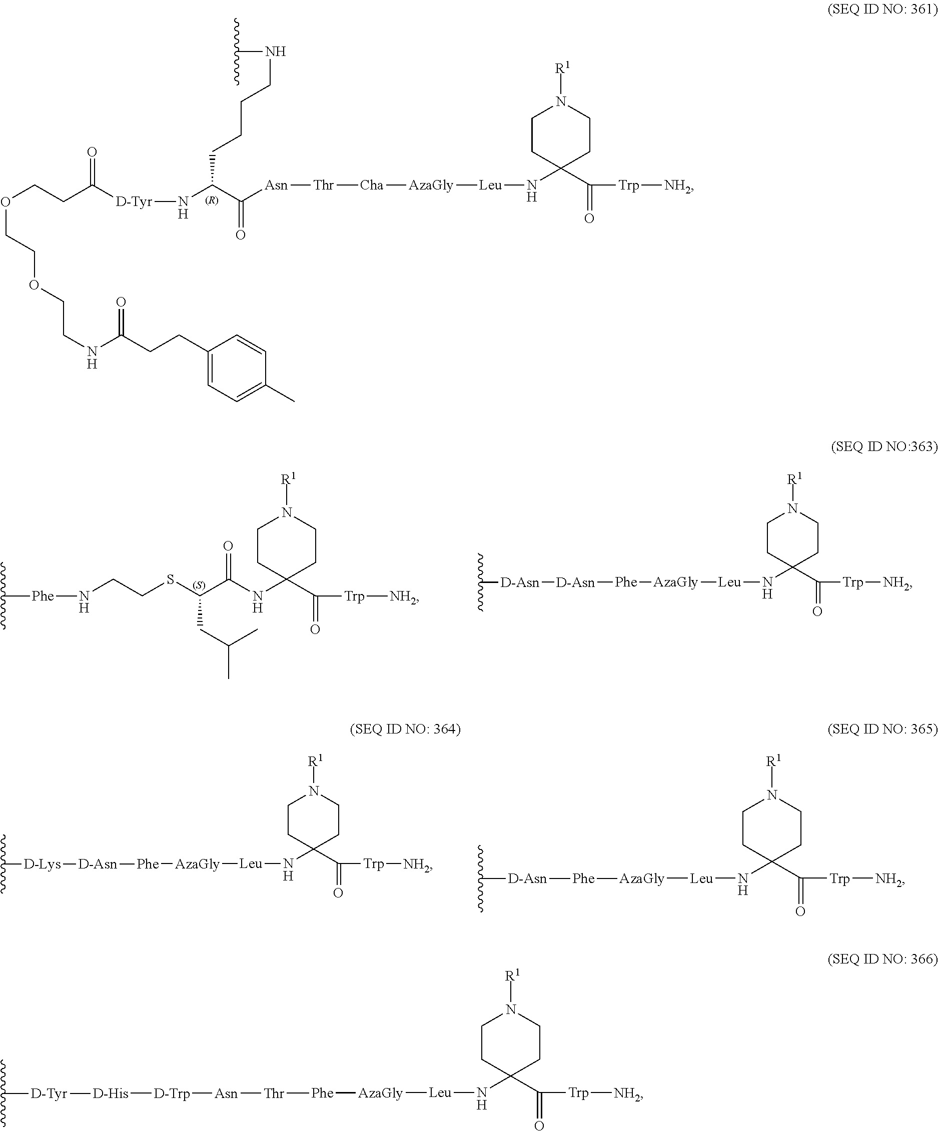

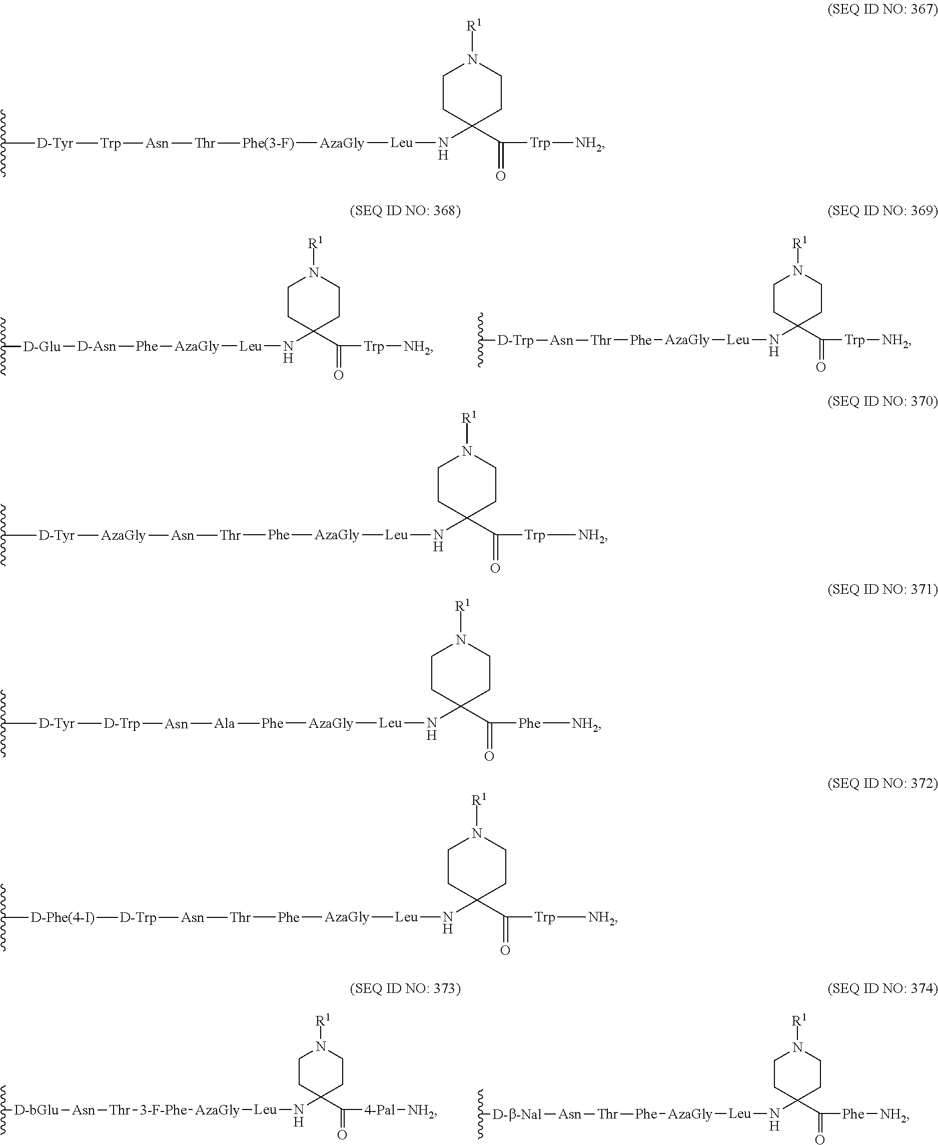

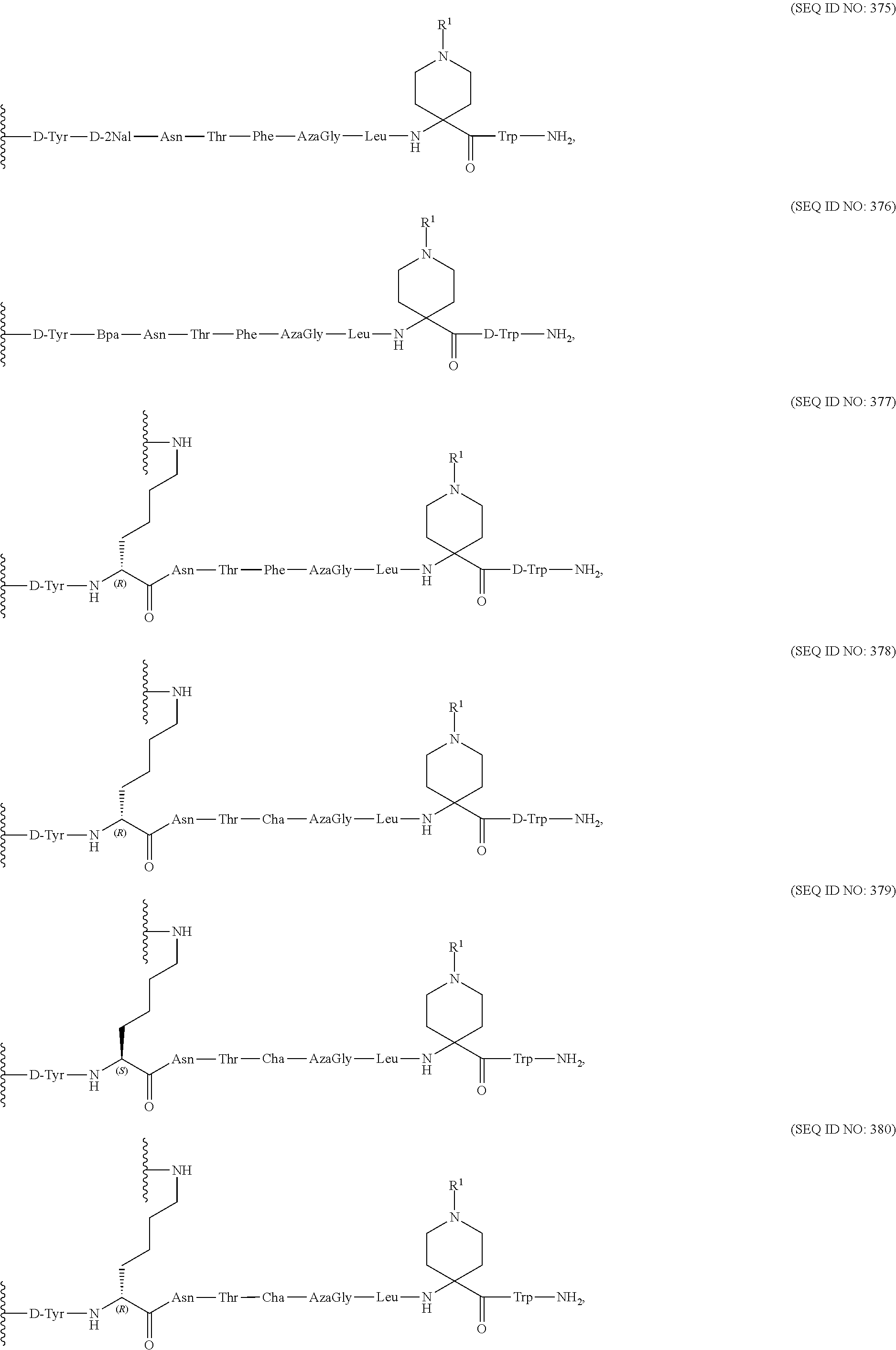

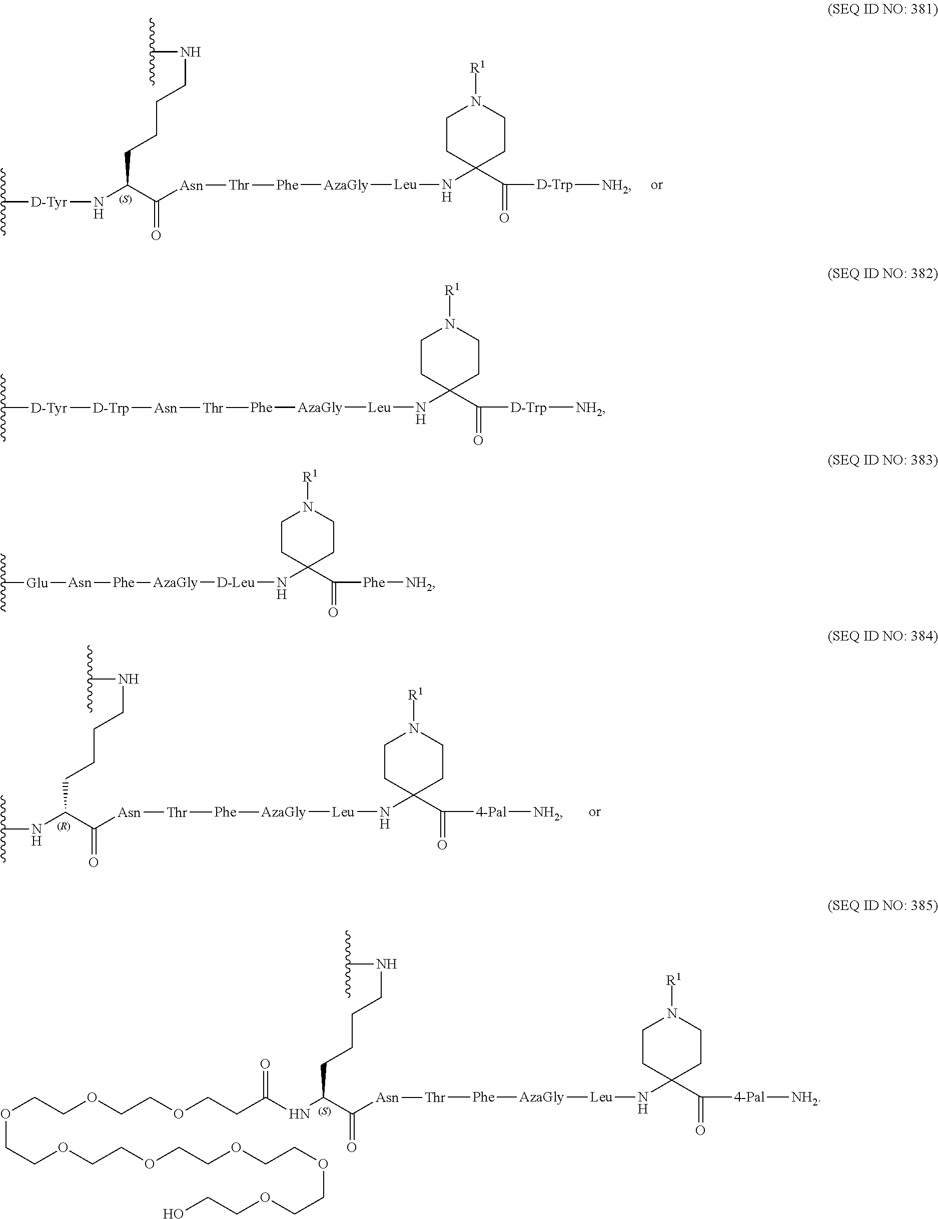

In some embodiments,

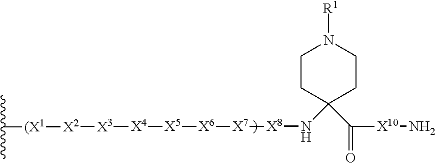

is

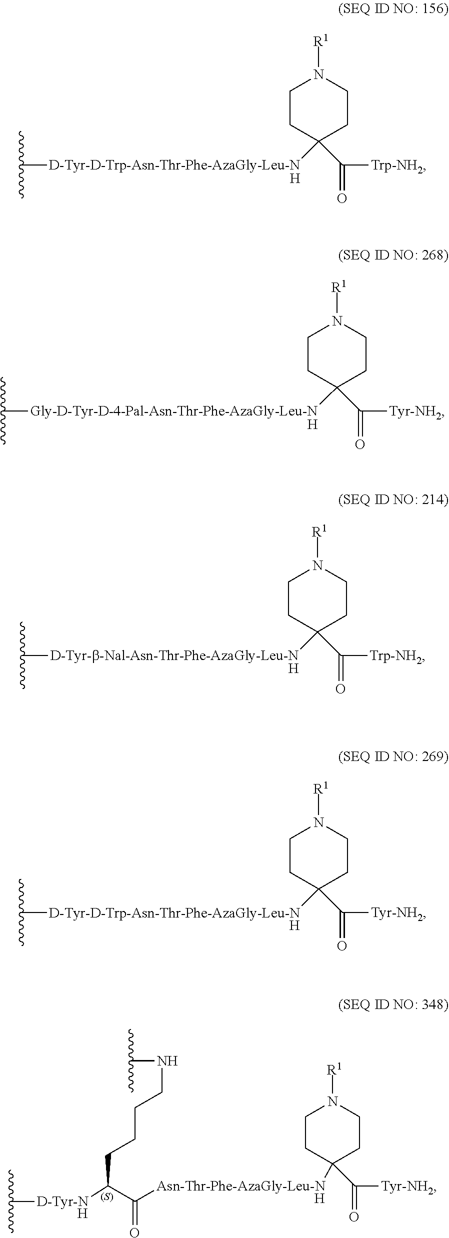

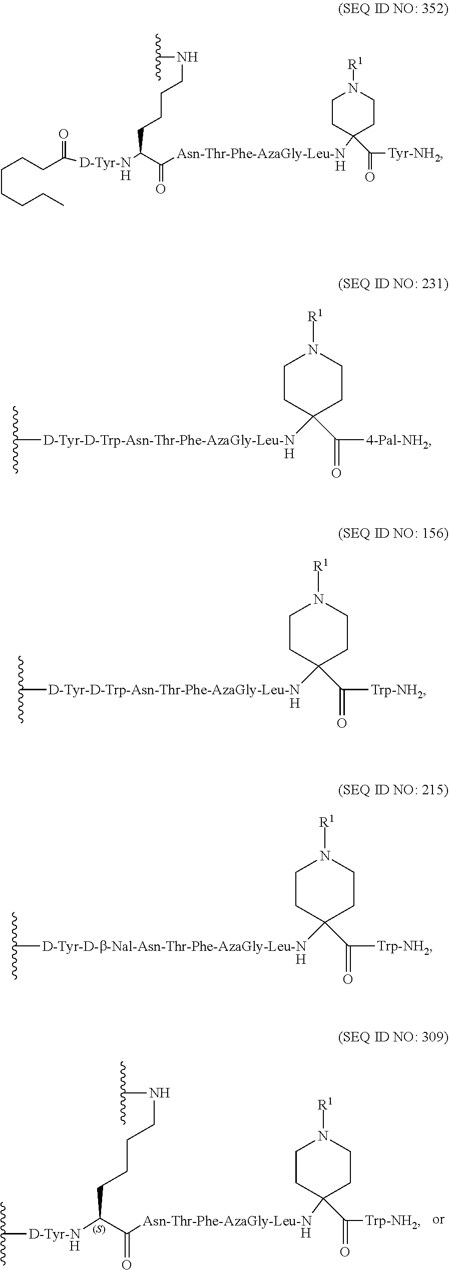

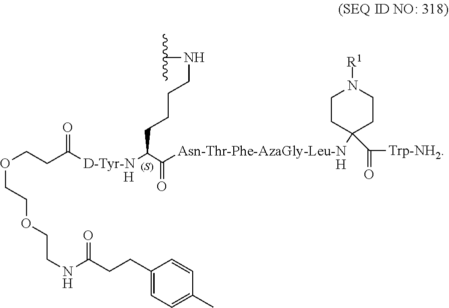

some embodiments of the previous embodiment R¹ is

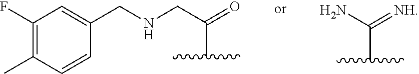

In some embodiments R¹ is

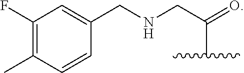 [SMILES: *C(=O)CNCc1ccc(C)c(F)c1]

In some embodiments R¹ is

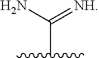 [SMILES: *C(=N)N]

In some embodiments R¹ is

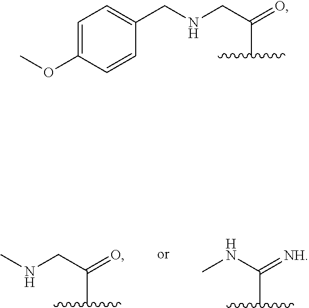

In some embodiments R¹ is

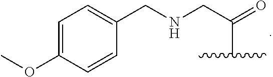 [SMILES: *C(=O)CNCc1ccc(OC)cc1]

In some embodiments R¹ is

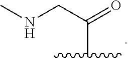 [SMILES: *C(=O)CNC]

In some embodiments R¹ is

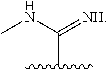 [SMILES: *C(=N)NC]

In some embodiments, the compound of Formula (I) has the following structure, or a pharmaceutically acceptable salt thereof:

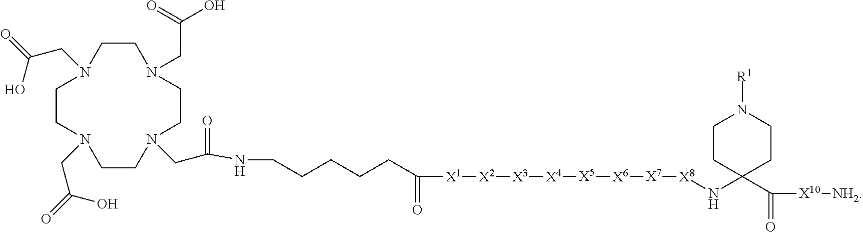

In some embodiments, the compound of Formula (I) has the following structure, or a pharmaceutically acceptable salt thereof:

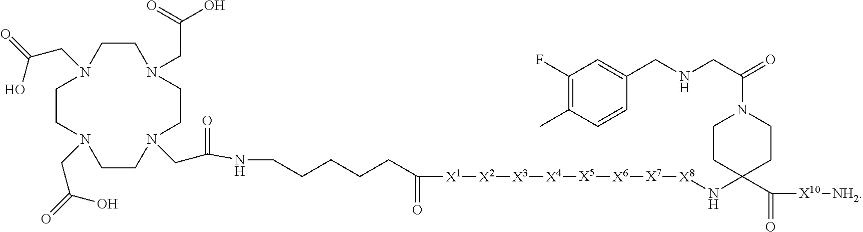

In some embodiments, the compound of Formula (I) has the following structure, or a pharmaceutically acceptable salt thereof:

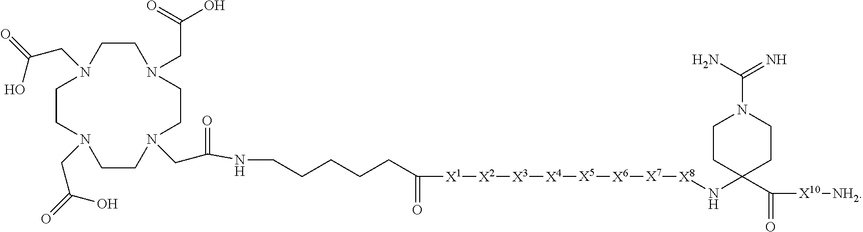

In some embodiments, the compound of Formula (I) has the following structure, or a pharmaceutically acceptable salt thereof:

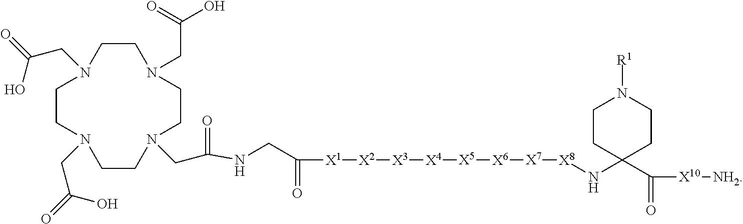

In some embodiments, the compound of Formula (I) has the following structure, or a pharmaceutically acceptable salt thereof:

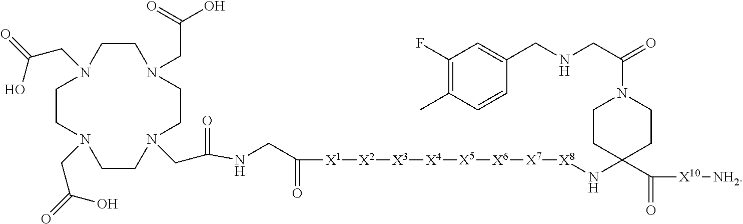

In some embodiments, the compound of Formula (I) has the following structure, or a pharmaceutically acceptable salt thereof:

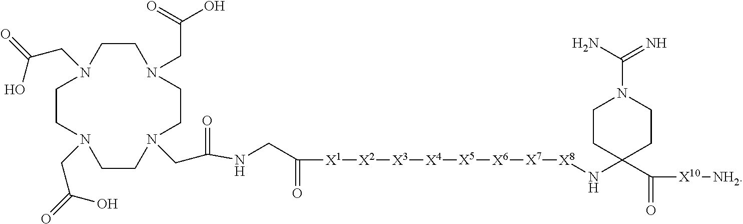

In some embodiments, the compound of Formula (I) has the following structure, or a pharmaceutically acceptable salt thereof:

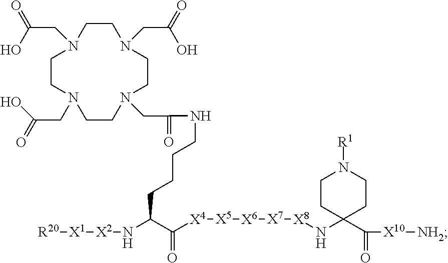

wherein R²⁰ is H, —C(═O)—C₁-C₂₀ alkyl, —C(═O)—(CH₂CH₂O)_{y}—CH₂CH₂—R¹⁵, —C₁-C₂₀ alkyl, N-hexadecanoyl-Glu, —C₄-C₂₀ polyethylene glycol, a saccharide, —C(═O)—(CH₂CH₂O)ₓ—CH₃, —C(═O)—(CH₂CH₂O)ₓ, —H, —C(═O)—CH₂CH₂CH(COOH)—R¹⁵, —C(═O)—(CH₂)₂R¹⁹, or —C(═O)CH₂NHCH₂R¹⁹. In some embodiments, the compound of Formula (I) has the following structure, or a pharmaceutically acceptable salt thereof:

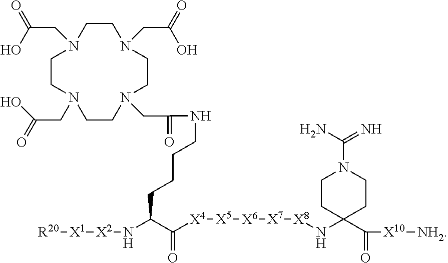

In some embodiments, the compound of Formula (I) has the following structure, or a pharmaceutically acceptable salt thereof:

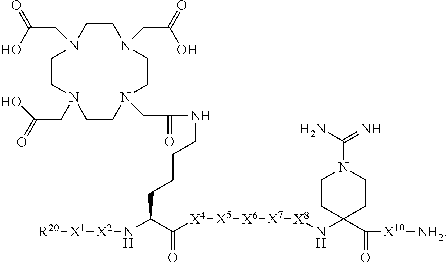

In some embodiments, the compound of Formula (I) has the following structure, or a pharmaceutically acceptable salt thereof:

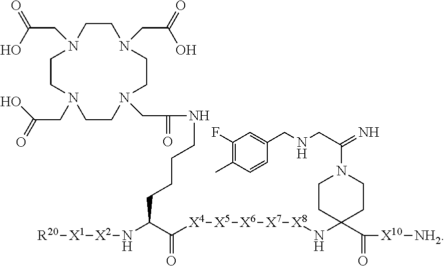

In some embodiments, R²⁰ is —C(═O)—C₁-C₁₀ alkyl. In some embodiments, R²⁰ is —C(═O)—(CH₂CH₂O)_{y}—CH₂CH₂—R¹⁵; wherein v is 1, 2, 3, or 4. In some embodiments, R¹⁵ is

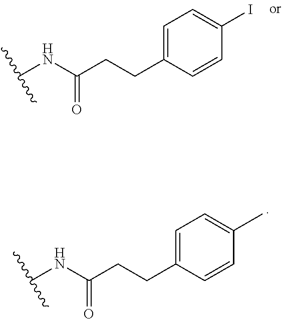 [SMILES: *NC(=O)CCc1ccc(I)cc1 ; *NC(=O)CCc1ccc(C)cc1]

### Radionuclide Complexes

Radiopharmaceuticals have increasingly become very useful tools for physicians to diagnose, stage, treat, and monitor the progression of several diseases, especially cancer. The primary difference between radiopharmaceuticals and other pharmaceutical drugs is that radiopharmaceuticals contain a radionuclide. The nuclear decay properties of the radionuclide determine whether a radiopharmaceutical will be used clinically as a diagnostic agent or as a therapeutic agent. Diagnostic radiopharmaceuticals require radionuclides that emit either gamma (γ) rays or positrons (β+), which subsequently annihilate with nearby electrons to produce two 511 keV annihilation photons emitted approximately 180° away from each other. Gamma ray-emitting radionuclides (e. g. ^{99m}Tc, ¹¹¹In, ²⁰¹TI, etc.) are useful for single photon emission computed tomography (SPECT), while positron-emitting radionuclides (e. g. ¹⁸F, 89Zr, ⁶⁸Ga, etc.) are useful for positron emission tomography (PET).

In contrast, therapeutic radiopharmaceuticals require radionuclides that emit particulate radiation, such as alpha (α) particles, beta (β−) particles, or Auger electrons. These particles, which strongly interact with target tissues (e. g. cancerous tumor) and lead to extensive localized ionization, can damage chemical bonds in DNA molecules and potentially induce cytotoxicity.

For most nuclear medicine applications, it is desired that a diagnostic radiopharmaceutical is paired with a therapeutic radiopharmaceutical. This concept is commonly known as “theranostics”. As a first step in the theranostic concept, a target molecule labeled with a diagnostic radionuclide is used for quantitative imaging of a tumor imaging biomarker, either by positron emission tomography (PET) or single photon emission computed tomography (SPECT). When it is demonstrated that, with this targeted molecule, a tumoricidal radiation absorbed dose can be delivered to tumor and metastases, as a second step, via administration of the same or a similar target molecule labeled with a therapeutic radionuclide.

In some embodiments, the chemical and pharmacokinetic behaviors of both the diagnostic and therapeutic radiopharmaceuticals match. In some embodiments, the diagnostic and therapeutic radionuclides are a chemically identical radioisotope pair (also known as a “matched pair”). One examples of a matched pair for theranostic radiopharmaceutical applications is the ¹²³I/¹³¹I pair, where ¹²³I-labeled compounds are used for diagnosis, while ¹³¹I-labeled compounds are used for therapy. Other theranostic matched pairs include ⁴⁴Sc/⁴⁷Sc, ⁶⁴Cu/⁶⁷Cu, ⁷²As/⁷⁷As, ⁸⁶Y/⁹⁰Y, and ²⁰³Pb/²¹²Pb, among others. Alternatively, radionuclide pairs from different elements can be utilized for theranostic radiopharmaceutical development when their chemistry is very similar (e.g. ^{99m}Tc/^{186/188}Re) and there is no significant difference in the pharmacokinetic behavior between the diagnostic and therapeutic analogues. Another example is the ⁶⁸Ga/¹⁷⁷Lu pair, where ⁶⁸Ga is used for diagnosis and ¹⁷⁷Lu is used for therapy. For example, gastroenteropancreatic endocrine tumors express high amounts of sst2 receptor that can be targeted with somatostatin receptor scintigraphy for diagnostic purposes with a ⁶⁸Ga sst2 ligand conjugate ([⁶⁸Ga]Ga-DOTA-TATE (NETSPOT™) or [⁶⁸Ga]Ga-DOTA-TOC (DOTA-(D-Phe1, Tyr3)-octreotide, SomaKit TOC®)), followed by treatment with a ¹⁷⁷Lu sst2 ligand conjugate ([¹⁷⁷Lu]Lu-DOTA-TATE) for endoradiotherapy.

### Chelating Moieties Used to Generate Metal (Radionuclide) Complexes

The compounds described herein comprise at least one R^{a} group, wherein R^{a} is a chelating moiety capable of chelating a radionuclide (Z′), or radionuclide complex thereof. In some embodiments, any suitable group or atom(s) of the chelator are used to connect, via an optional linker, to the KISS1R targeting ligand.

In some embodiments, the chelator is capable of binding a radioactive atom. In some embodiments, the binding is direct, e.g., the chelator makes hydrogen bonds or electrostatic interactions with a radioactive atom. In some embodiments, the binding is indirect, e.g., the chelator binds to a molecule that comprises a radioactive atom. In some embodiments, the chelator is or comprises a macrocycle.

In some embodiments, the chelator comprises one or more amine groups. In some embodiments, the metal chelator comprises two or more amine groups. In some embodiments, the chelator comprises three or more amine groups. In some embodiments, the chelator comprises four or more amine groups. In some embodiments, the chelator includes 4 or more N atoms, 4 or more carboxylic acid groups, or a combination thereof. In some embodiments, the chelator does not comprise S. In some embodiments, the chelator comprises a ring. In some embodiments, the ring comprises an O and/or a N atom. In some embodiments, the chelator is a ring that includes 3 or more N atoms, 3 or more carboxylic acid groups, or a combination thereof. In some embodiments, the chelator is polydentate ligand, bidentate ligand, or monodentate ligand. Polydentate ligands range in the number of atoms used to bond to a metal atom or ion. EDTA, a hexadentate ligand, is an example of a polydentate ligand that has six donor atoms with electron pairs that can be used to bond to a central metal atom or ion. Bidentate ligands have two donor atoms which allow them to bind to a central metal atom or ion at two points. Ethylenediamine (en) and the oxalate ion (ox) are examples of bidentate ligands.

In some embodiments, a chelator described herein comprises a cyclic chelating agent or an acyclic chelating agent. In some embodiments, a chelator described herein comprises a cyclic chelating agent. In some embodiments, a chelator described herein comprises an acyclic chelating agent.

In some embodiments, a chelator described herein comprises cyclen, DO2A, DO3A, HP-DO3A, DO3A-Nprop, DO3 AP, DO3A^{PrA}, DO3AP^{ABn}, DO3AM^{nBu} BT-DO3A, DOTA, PSC, DOTAGA, DOTA(GA)₂, DOTAM, DOTA-4AMP, DOTMA, DOTP, CB-DO2A, DOTPA, DOTMP, DOTAMAP, TRITA, L^{py}, cyclam, TETA, CB-Cyclam, CB-TE2A, TE2A, NOTA, NODAGA, NODA-MPAA, TACN, TACN-TM, NOTP, Sarcophagine (Sar), DiAmSar, SarAr, AmBaSar, cis-DO2A2P, trans-DO2A2P, DOTEP, p-NO₂-Bn-DOTA, BAT, DO3TMP-Monoamide, CHX-A″-DTPA, c-DEPA, PCTA, p-NO₂-Bn-PCTA, TRAP, TRAPH, TRAP-OH, TRAP-Ph, NOPO, AAZTA, DATAM, HEHA, PEPA, DTA, EDTMP, DTPMP, NTA, EDTA, DTPA, CyDTPA, DFO, DFO*, deferiprone, TTHA, HBED, HBED-CC, HBED-CC TFP, H₄pypa, H₄py4pa, CP256, THP, YM103, t-Bu-calix[4]arene-tetracarboxylic acid, CHX-A″-DTPA, H₆phospha, p-NH₂-Bn-CHXA″-DTPA, DEDPA, H₄octox, H₄octapa, H₄CHXoctapa, HYNIC, macropa, crown, macropid, HOPO, Bis(2-mercaptoacetamide), Bis(aminothiolate), or SBTG₂DAP.

In some embodiments, a chelator described herein comprises DOTA, DOTAGA, DOTA(GA)₂, NOTA, NODAGA, TRITA, TETA, DOTA-MA, HP-DO3A, DOTMA, DOTA-pNB, DOTP, DOTMP, DOTEP, DOTMPE, F-DOTPME, DOTPP, DOTBzP, DOTA-monoamide, BAT, DO3TMP-Monoamide, and CHX-A″-DTPA.

In some embodiments, a chelator described herein comprises DTA, CyEDTA, EDTMP, DTPMP, DTPA, CyDTPA, Cy2DTPA, DTPA-MA, DTPA-BA, and BOPA.

In some embodiments, a chelator described herein comprises DOTA, DOTAGA, DOTA(GA)₂, DOTP, DOTMA, DOTAM, DTPA, NTA, EDTA, DO3A, DO2A, NOC, NOTA, TETA. TACN, DiAmSar, CB-Cyclam, CB-TE2A, DOTA-4AMP, or NOTP.

In some embodiments, a chelator described herein comprises HP-DO3A, BT-DO3A, DO3A-Nprop, DO3AP, DO2A2P, DOA3P, DOTP, DOTPMB, DOTAMAE, DOTAMAP, DO3AM^{Bu}, DOTMA, TCE-DOTA, DEPA, PCTA, p-NO₂-Bn-PCTA, p-NO₂-Bn-DOTA, symPC2APA, symPCA2PA, asymPC2APA, asymPCA2PA, TRAP, AAZTA, DATA^{m}, THP, HEHA, HBED, or HBED-CC TFP.

In some embodiments, a chelator described herein comprises DOTA, NOTA, NODAGA, DOTAGA, HBED, HBED-CC TFP, H2DEPDPA, DFO-B, Deferiprone, CP256, YM103, TETA, CB-TE2A, TE2A, Sar, DiAmSar, TRAPH, TRAP-Pr, TRAP-OH, TRAP-Ph, NOPO, DEADPA, PCTA, EDTA, PEPA, HEHA, DTPA, EDTMP, AAZTA, DO3 AP, DO3AP^{PrA}, DO3AP^{ABn}, or DOTAM.

In some embodiments, the chelator is or comprises DOTA, HBED-CC, DOTAGA, DOTA(GA)₂, NOTA, and DOTAM. In some embodiments, the chelator is or comprises NODAGA, NOTA, DOTAGA, DOTA(GA)₂, TRAP, NOPO, NCTA, DFO, DTPA, and HYNIC.

In some embodiments, the chelator comprises a macrocycle, e.g., a macrocycle comprising an O and/or a N atom, DOTA, HBED-CC, DOTAGA, DOTA(GA)₂, NOTA, DOTAM, one or more amines, one or more ethers, one or more carboxylic acids, EDTA, DTPA, TETA, DO3A, PCTA, or desferrioxamine.

In some embodiments, a metal chelator described herein comprises one of the following structures:

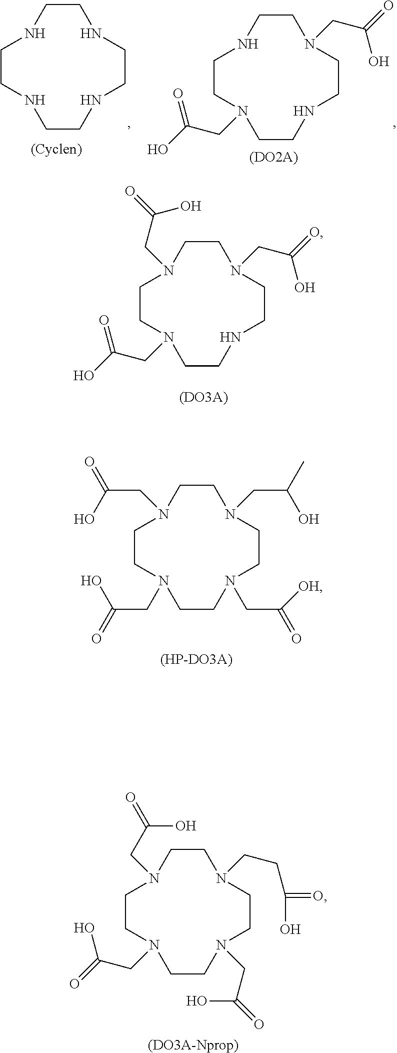

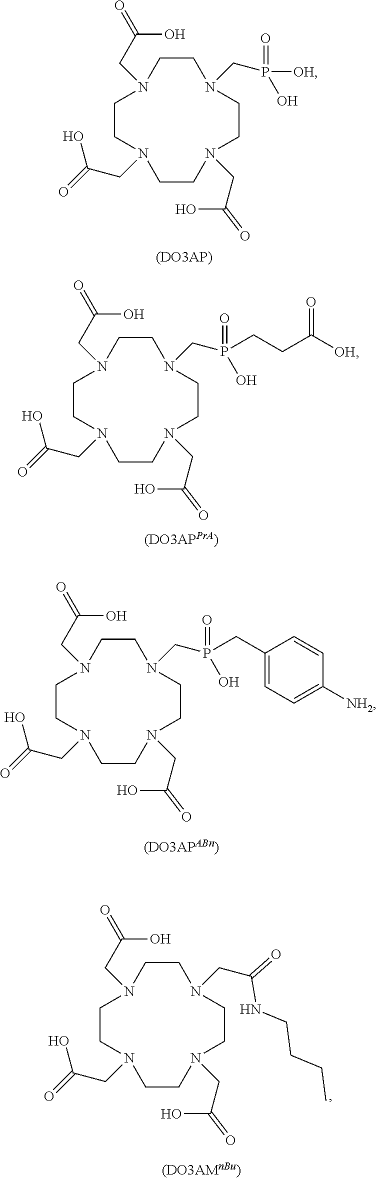

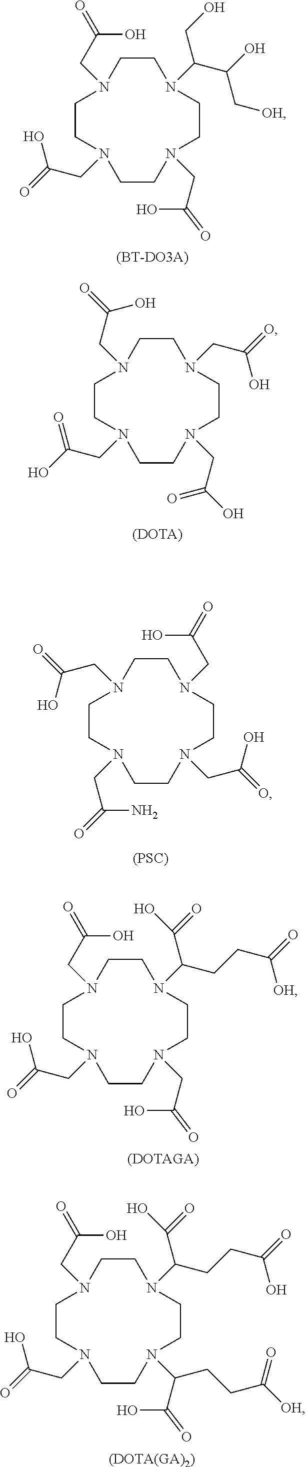

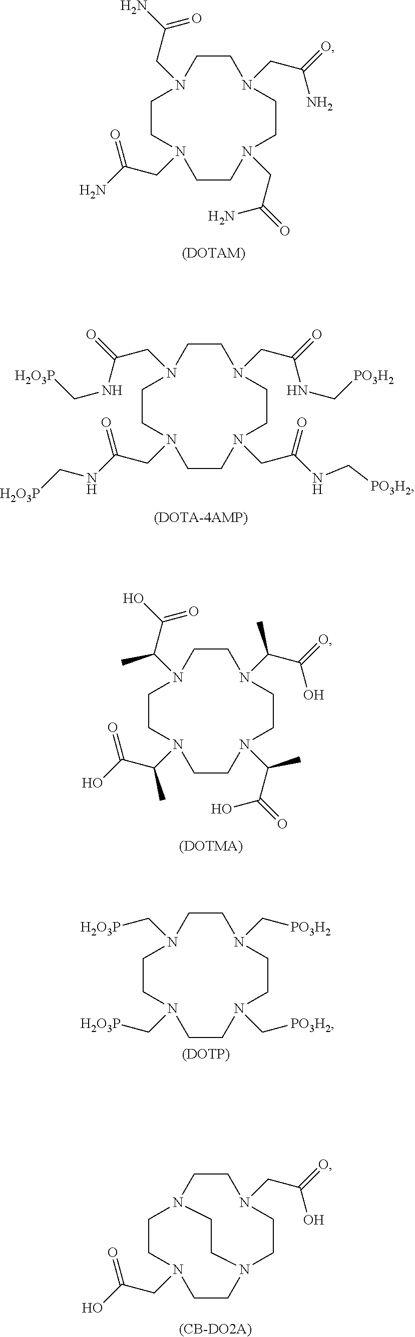

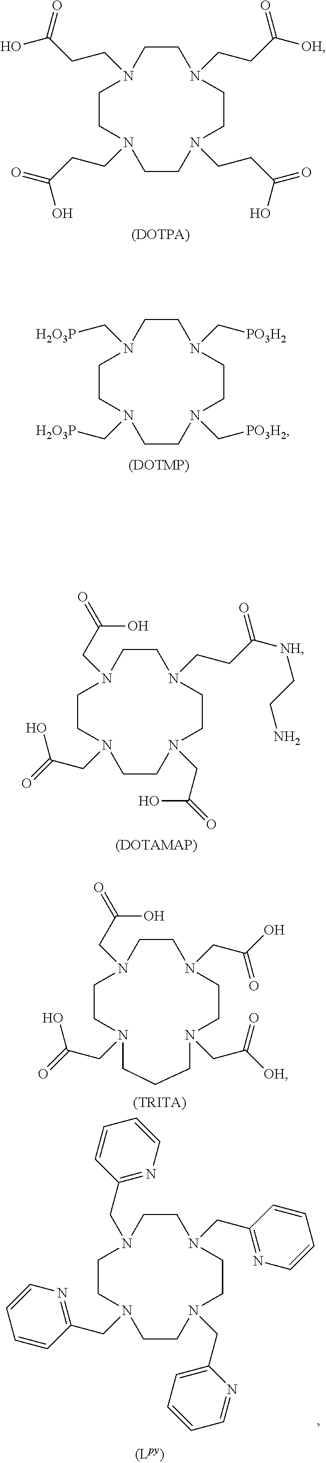

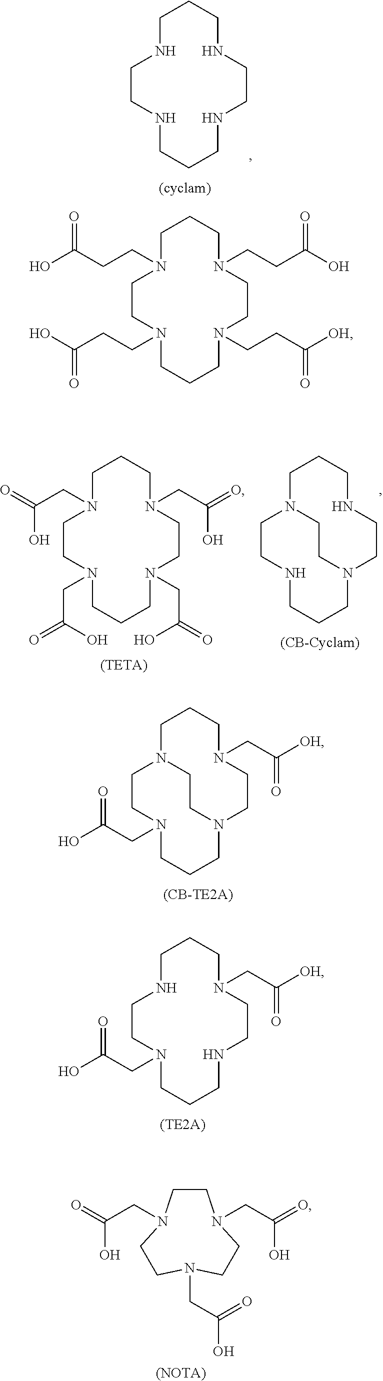

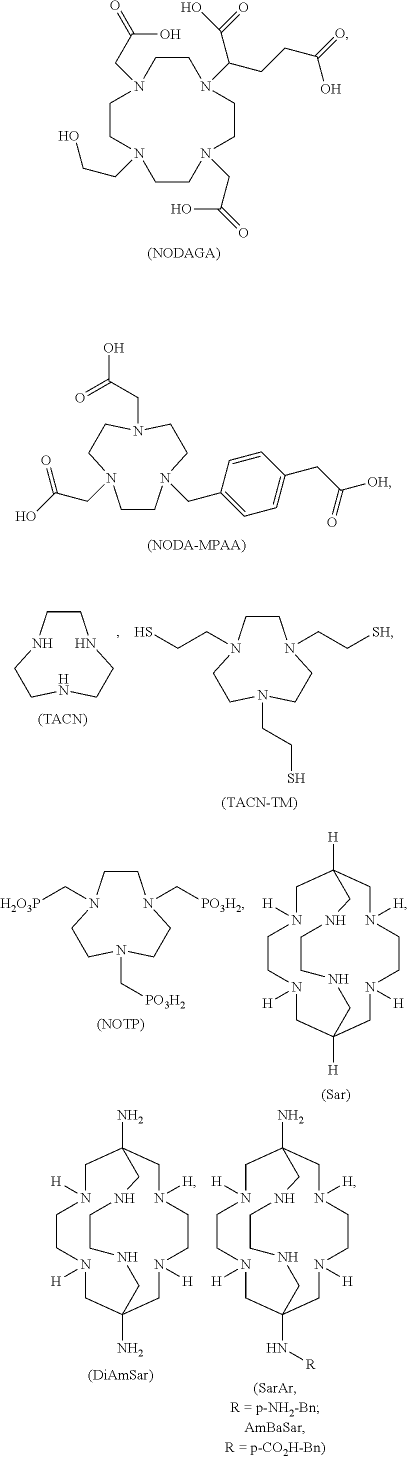

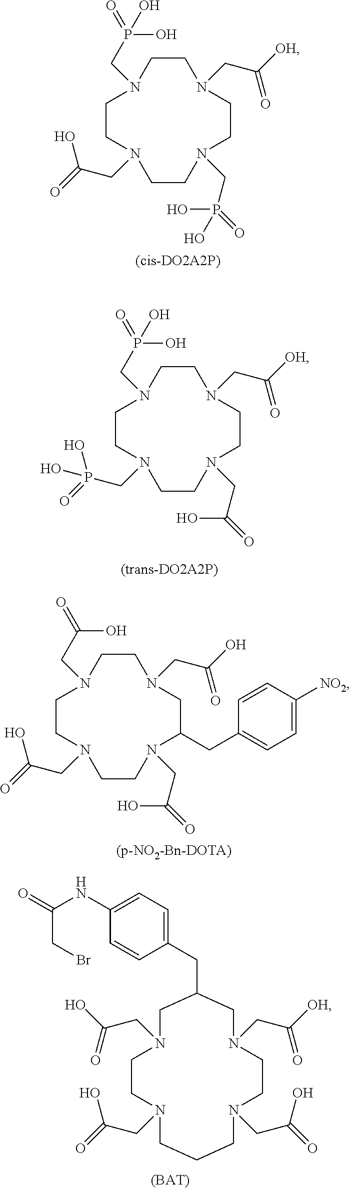

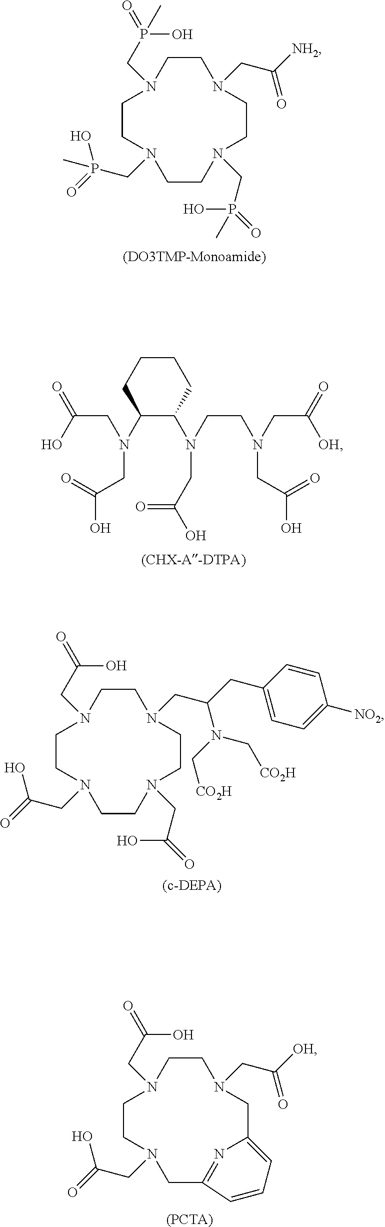

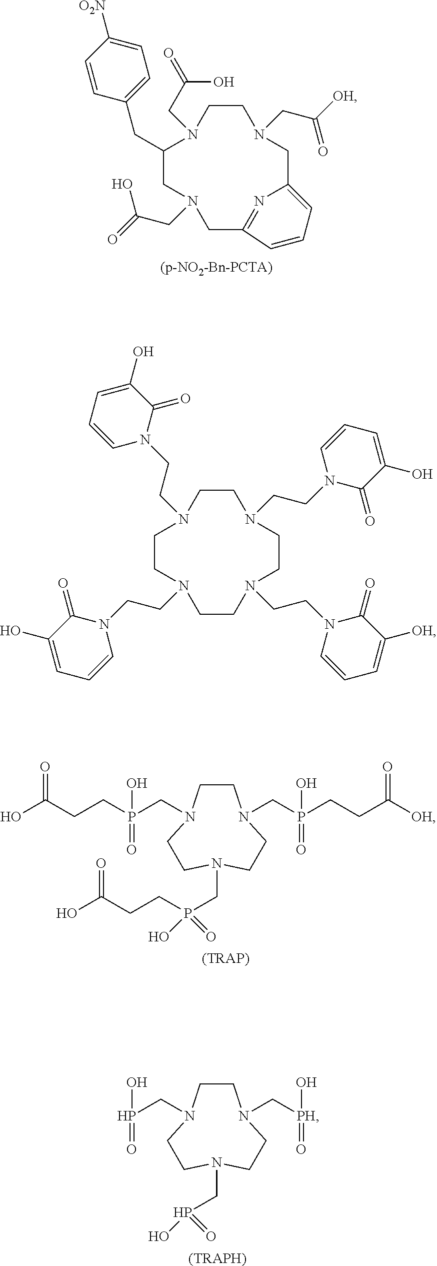

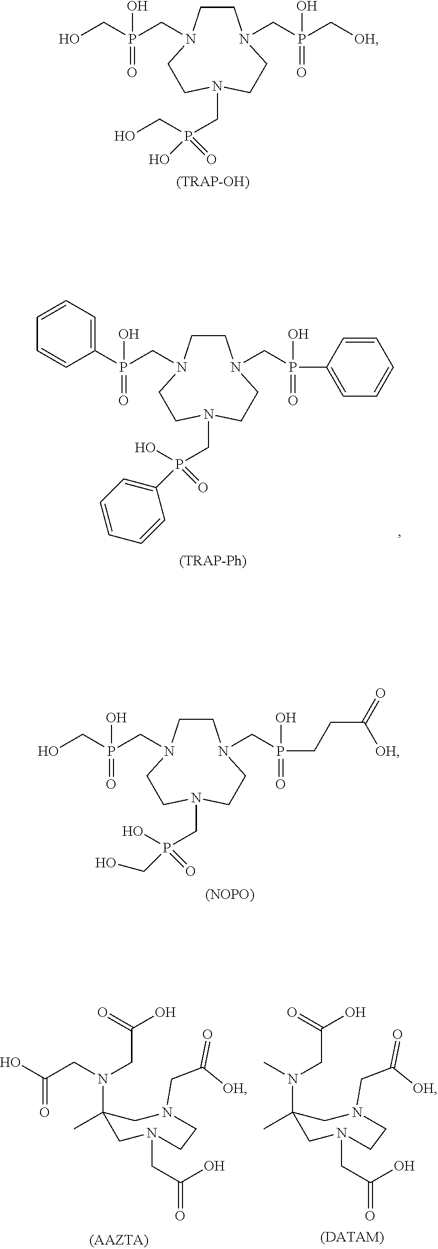

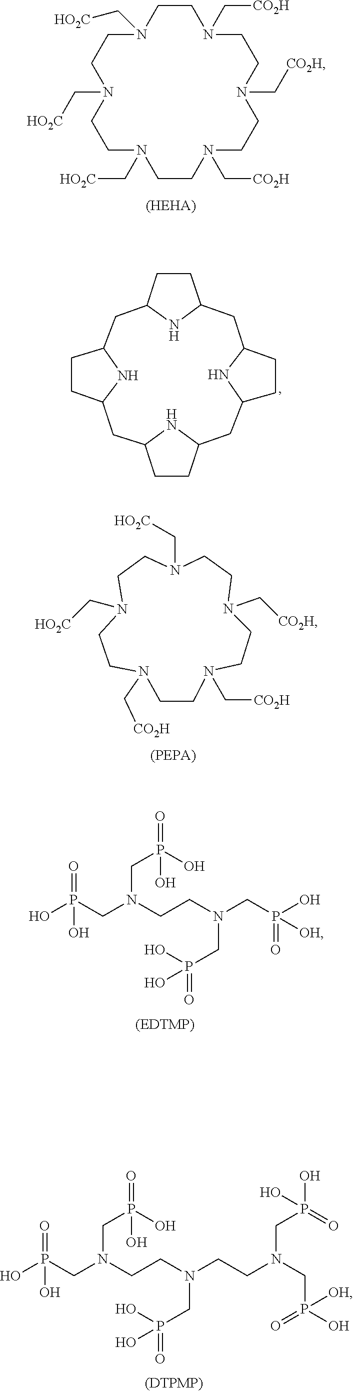

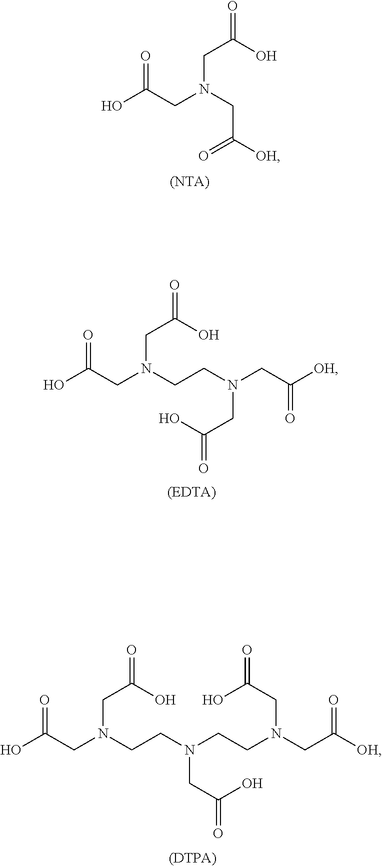 [SMILES: O=C(O)CN(CC(=O)O)CC(=O)O ; O=C(O)CN(CCN(CC(=O)O)CC(=O)O)CC(=O)O ; O=C(O)CN(CCN(CC(=O)O)CC(=O)O)CCN(CC(=O)O)CC(=O)O]

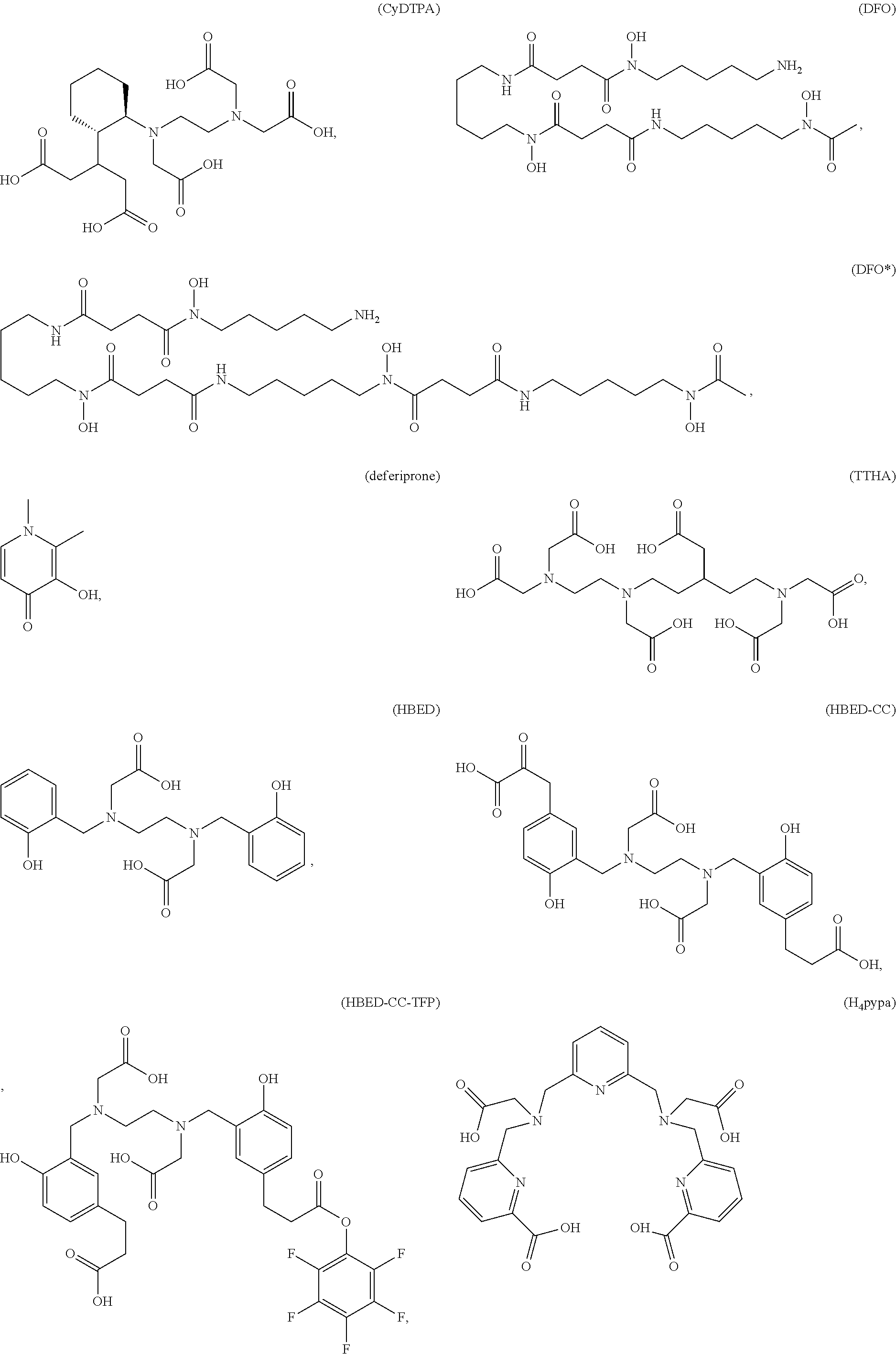

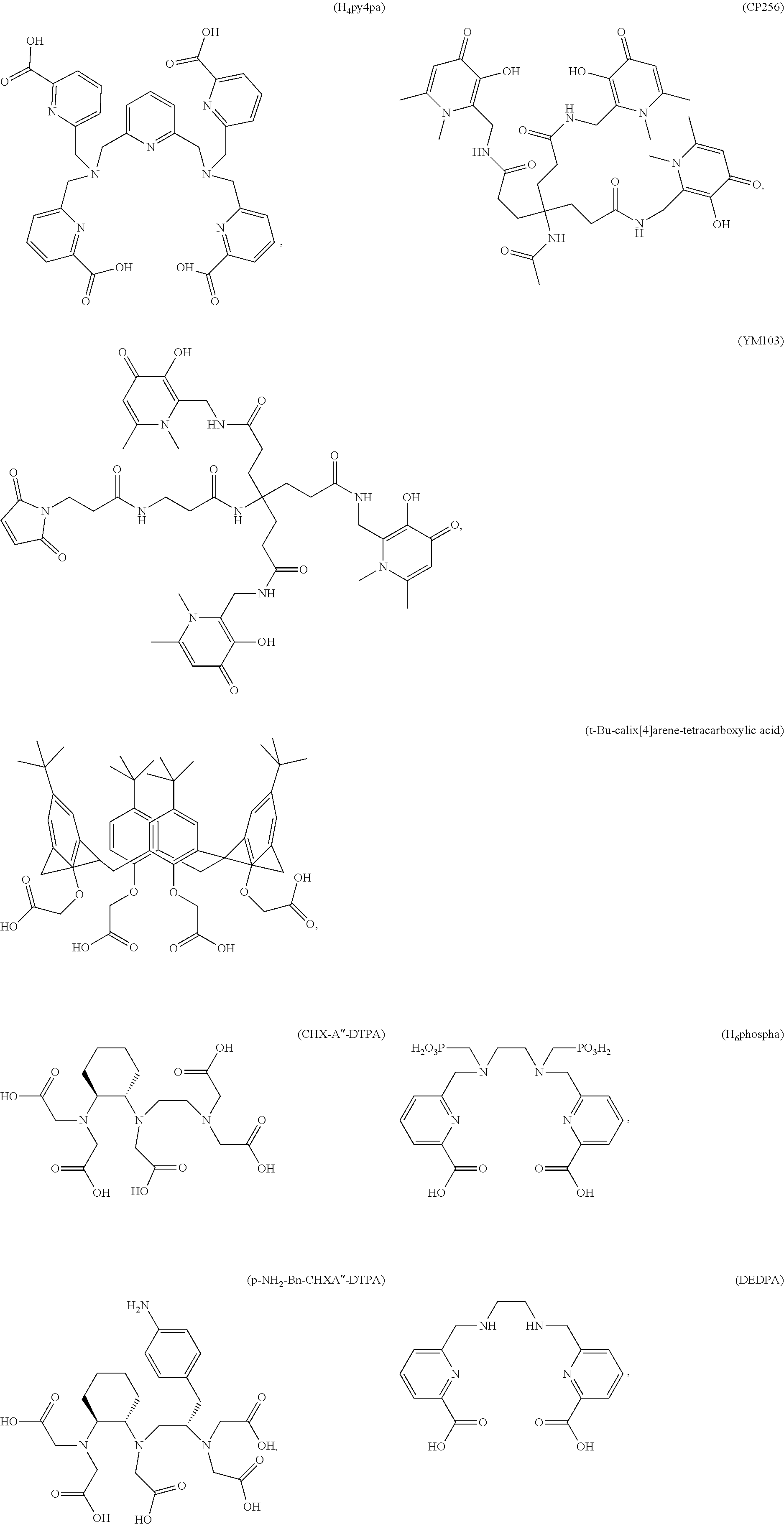

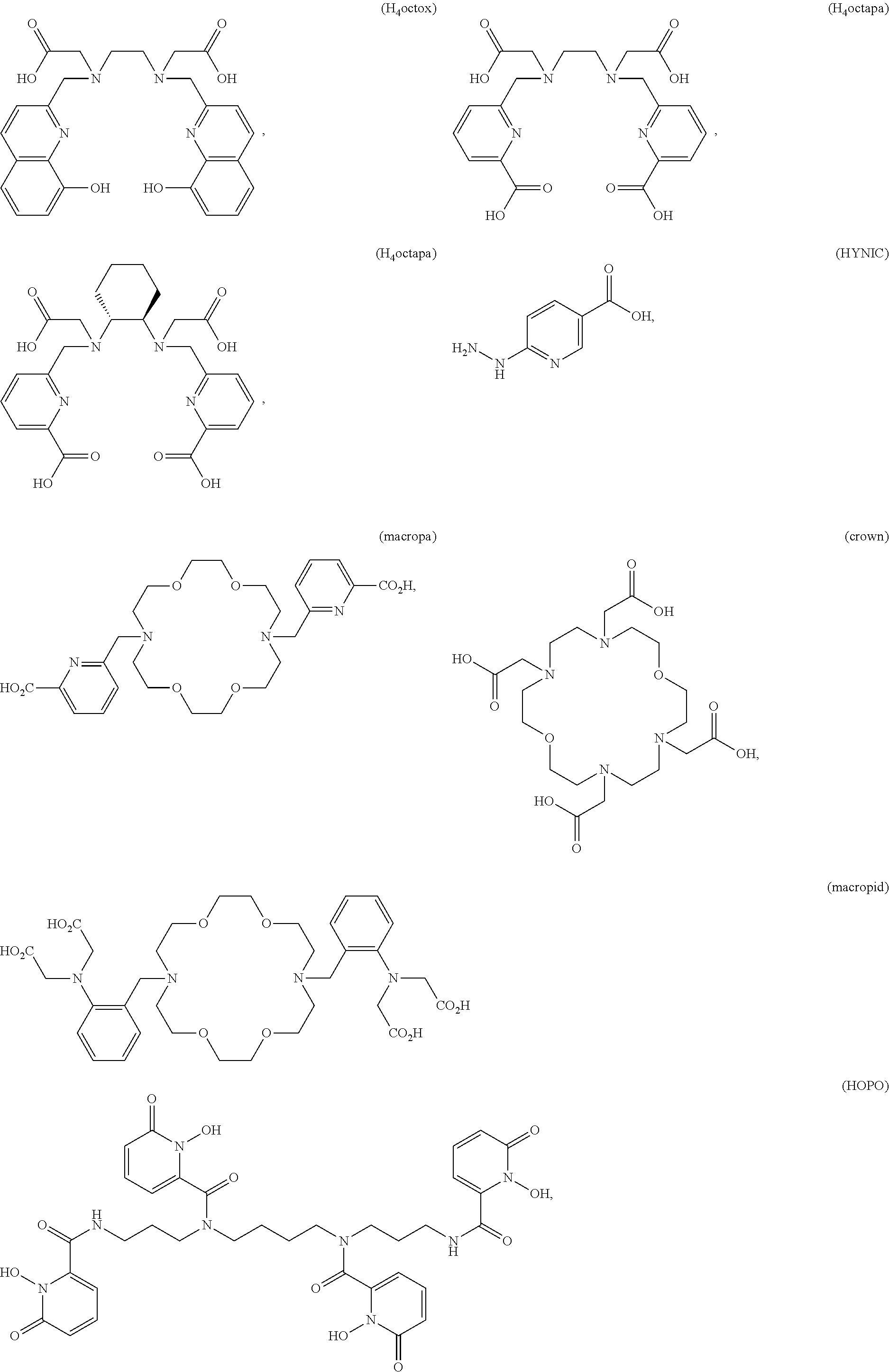

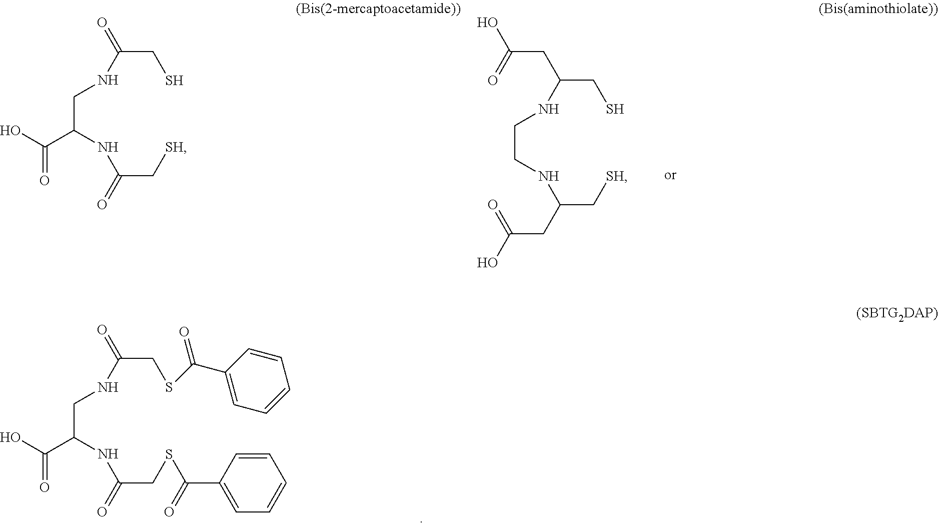 [SMILES: O=C(CS)NCC(NC(=O)CS)C(=O)O ; O=C(O)CC(CS)NCCNC(CS)CC(=O)O ; "or" ; O=C(CSC(=O)c1ccccc1)NCC(NC(=O)CSC(=O)c1ccccc1)C(=O)O]

In some embodiments, the chelating moiety R^{a} comprises a radionuclide and DOTA. In some embodiments, the chelating moiety R^{a} comprises a radionuclide and a DOTA derivative. In some embodiments, the chelating moiety comprises two independent chelators, and at least one or both are DOTA.

In some embodiments, the chelating moiety comprises a radionuclide and a chelator configured to bind the radionuclide (Z′), wherein the chelator comprises DOTA, DOTP, DOTMA, DOTAM, DTPA, NOTA, NTA, NODAGA, EDTA, DO3A, DO2A, NOC, TETA, CB-TE2A, DiAmSar, CB-Cyclam, DOTA-4AMP, H₄pypa, H₄octox, H₄octapa, p-NO₂-Bn-neunpa, or NOTP.

In some embodiments, the metal chelator described herein comprises macropa or crown. In some embodiments, the metal chelator described herein comprises macropa. In some embodiments, the metal chelator described herein comprises crown. In some embodiments, the metal chelator described herein comprises

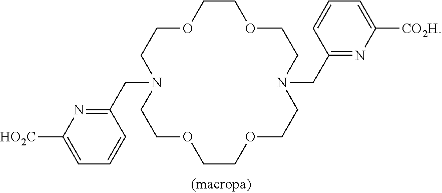 [SMILES: O=C(O)c1cccc(CN2CCOCCOCCN(Cc3cccc(C(=O)O)n3)CCOCCOCC2)n1]

In some embodiments, the metal chelator described herein comprises

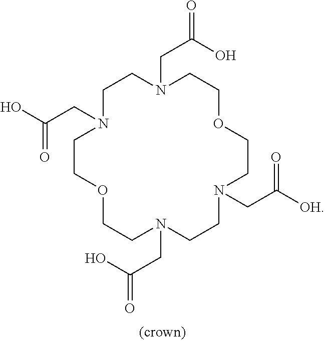 [SMILES: O=C(O)CN1CCOCCN(CC(=O)O)CCN(CC(=O)O)CCOCCN(CC(=O)O)CC1]

In some embodiments, the chelating moiety of R^{a} is independently selected from the group consisting of: cyclen, DO2A, DO3A, HP-DO3A, DO3 A-Nprop, DO3AP, D3 AP^{PrA}, DO3AP^{ABn}, DO3AM^{nBu}, BT-DO3 A, DOTA, DOTAGA, DOTA(GA)₂, DOTAM, DOTA-4AMP, DOTMA, DOTP, CB-DO2A, DOTPA, DOTMP, DOTAMAP, TRITA, L^{py}, cyclam, TETA, CB-Cyclam, CB-TE2A, TE2A, NOTA, NODAGA, NO)A-MPAA, TACN, TACN-TM, NOTP, Sarcophagine (Sar), DiAmSar, SarAr, AmBaSar, cis-DO2A2P, trans-DO2A2P, DOTEP, p-NO₂-Bn-DOTA, BAT, DO3TMP-Monoamide, CHX-A″-DTPA, c-DEPA, PCTA, p-NO₂-Bn-PCTA, TRAP, TRAPH, TRAP-OH, TRAP-Ph, NOPO, AAZTA, DATAM, HEHA, PEPA, DTA, EDTMP, DTPMP, NTA, EDTA, DTPA, CyDTPA, DFO, DFO*, deferiprone, TTHA, HBED, HBED-CC, HBED-CC TFP, H₄pypa, H₄py4pa,CP256, TRP, YM103, t-Bu-calix[4]arene-tetracarboxylic acid, CHX-A″-DTPA, H₆phospha, p-NH₂-Bn-CHXA″-DTPA, DEDPA, H₄octox, H₄octapa, H₄CHXoctapa, HYNIC, macropa, crown, macropid, HOPO, Bis(2-mercaptoacetamide), Bis(aminothiolate), and SBTG₂DAP.

In some embodiments, R^{a} is a chelating moiety selected from the group consisting of: DOTA; 2,2′,2″-(10-(2-amino-²-oxoethyl)-1,4,7,10-tetraazacyclododecane-1,4,7-triylitriacetic acid (PSC); DO3A; DO2A; DOTMA; DOTAM; DOTPA; 2,2′,2″-(10-(2-amino-2-oxoethyl)-1,4,7,10-tetraazacyclododecane-1,4,7-triyl)triacetic acid; Bn-DOTA; p-OH-Bn-DOTA; H₄pypa; H₄pypa-benzyl; H₄py4pa; H₄py4pa-benzyl; NOTA; macropa; crown; H₄octapa; H₄octapa-benzyl; and TTHA; or a radionuclide complex thereof.

In some embodiments, the chelating moiety of R^{a} is independently selected from the group consisting of: 1,4,7,10-tetraazacyclododecane-1,4,7,10-tetraacetic acid (DOTA);
1,4,7,10-tetraazacyclododecane-1,4,7-triacetic acid (DO3A);
1,4,7,10-tetraazacyclododecane-1,7-diacetic acid (DO2A);
   α,α′″,α″′-tetramethyl-1,4,7,10-tetraazacyclododecane-1,4,7,10-tetraacetic acid (DOTMA);
1,4,7,10-tetrakis(carbamoylmethyl)-1,4,7,10-tetraazacyclododecane (DOTAM);
1,4,7,10-tetraazacyclododecane-1,4,7,10-tetrapropionic acid (DOTPA);
   2,2′,2-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA);
   p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyI))bis(methylene))dipicolinic acid (H₄pypa); H₄pypa-benzyl;
   6,6′,6″,6″′-(((pyridine-2,6-diylbis(methylene))bis(azanetriyl))tetrakis(methylene))-tetrapicolinic acid (H₄py4pa); H₄py4pa-benzyl;
   2,2′,2″-(1,4,7-triazacyclononane-1,4,7-triyl)triacetic acid (NOTA);
   6,6′-((1,4,10,13-tetraoxa-7,16-diazacyclooctadecane-7,16-diyl)bis(methylene))dipicolinic acid (macropa);
   2,2′,2′″-(1,10-dioxa-4,7,13,16-tetraazacyclooctadecane-4,7,13,16-tetrayl)tetraacetic acid (crown);
   6,6′-((ethane-1,2-diylbis((carboxymethyl)azanediyl))bis(methylene))dipicolinic acid (H₄octapa); H₄octapa-benzyl; and
3,6,9,12-tetrakis(carboxymethyl)-3,6,9,12-tetraazatetradecanedioic acid (TTHA); or a radionuclide complex thereof.

In some embodiments, the chelating moiety of R^{a} is independently selected from the group consisting of: DOTA; DO3A, DO2A; DOTMA; DOTAM; DOTPA; 2,2,2″-(10-(2-amino-2-oxoethyl)-1,4,7,10-tetraazacyclododecane-1,4,7-triyl)triacetic acid; H₄pypa; H₄py4pa; NOTA; macropa; crown; H₄octapa; and TTHA; or a radionuclide complex thereof.

In some embodiments, R^{a} is DOTA or a radionuclide complex thereof. In some embodiments, R^{a} is DO3A or a radionuclide complex thereof. In some embodiments, R^{a} is DO2A or a radionuclide complex thereof. In some embodiments, R^{a} is DOTMA or a radionuclide complex thereof. In some embodiments, R^{a} is DOTAM or a radionuclide complex thereof. In some embodiments, R^{a} is DOTPA or a radionuclide complex thereof. In some embodiments, R^{a} is 2,2,2″-(10-(2-amino-2-oxoethyl)-1,4,7,10-tetraazacyclododecane-1,4,7-triyl)triacetic acid or a radionuclide complex thereof. In some embodiments, R^{a} is H₄pypa or a radionuclide complex thereof. In some embodiments, R^{a} is 1H₄py4pa or a radionuclide complex thereof. In some embodiments, R^{a} is NOTA or a radionuclide complex thereof. In some embodiments, R^{a} is macropa or a radionuclide complex thereof. In some embodiments, R^{a} is crown or a radionuclide complex thereof. In some embodiments, R^{a} is H₄octapa or a radionuclide complex thereof. In some embodiments, R^{a} is TTHA or a radionuclide complex thereof.

In some embodiments, R^{a} is: DOTA or DO3A; or a radionuclide complex thereof.

In some embodiments, the chelating moiety of R^{a} is independently selected from the group consisting of: DOTA; DO3DA DO2A; DOTMA; DOTAM; DOTPA; 2,2′,2″-(10-(2-amino-2-oxoethyl)-1,4,7,10-tetraazacyclododecane-1,4,7-triyl)triacetic acid; H₄pypa; H₄py4pa; NOTA; macropa; crown; H₄octapa; and TTHA; or a radionuclide complex thereof.

In some embodiments, R^{a} is DOTA or a radionuclide complex thereof. In some embodiments, R^{a} is DO)₃A or a radionuclide complex thereof. In some embodiments, R^{a} is DO2A or a radionuclide complex thereof. In some embodiments, R^{a} is DOTMA or a radionuclide complex thereof. In some embodiments, R^{a} is DOTAM or a radionuclide complex thereof. In some embodiments, R^{a} is DOTPA or a radionuclide complex thereof. In some embodiments, R^{a} is 2,2′,2″10-(2-amino-2-oxoethyl)-1,4,7,10-tetraazacyclododecane-1,4,7-triyl)triacetic acid or a radionuclide complex thereof. In some embodiments, R^{a} is H₄pypa or a radionuclide complex thereof. In some embodiments, R^{a} is H₄py4pa or a radionuclide complex thereof. In some embodiments, R^{a} is NOTA. In some embodiments, R^{a} is macropa. In some embodiments, R^{a} is crown. In some embodiments, R^{a} is H₄octapa or a radionuclide complex thereof. In some embodiments, R^{a} is TTHA or a radionuclide complex thereof.

In some embodiments, the chelating moiety of R^{a} is: DOTA or DO3A; or a radionuclide complex thereof.

In some embodiments, R^{a} is a chelating moiety selected from the group consisting of:

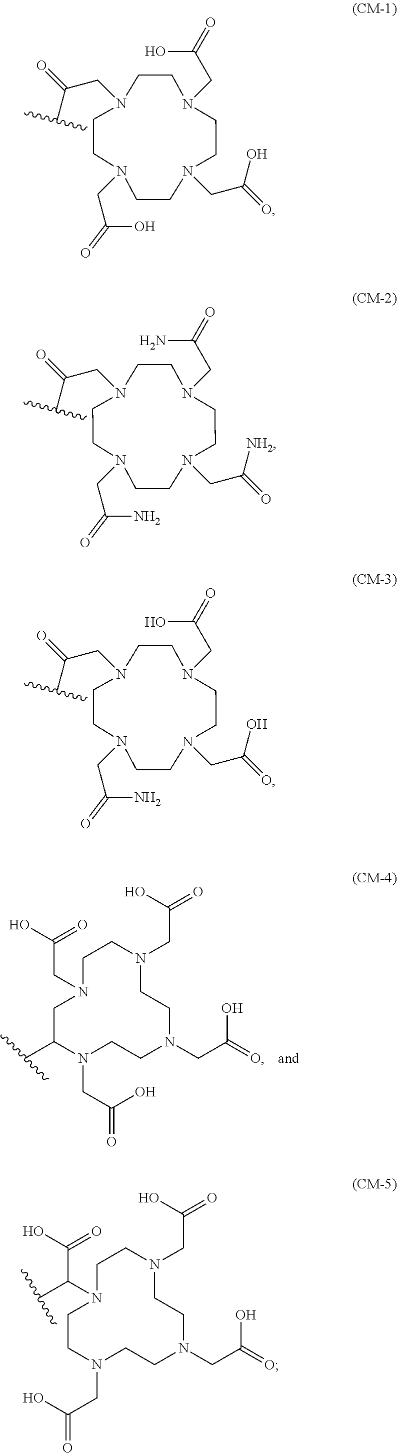 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

In some embodiments, R^{a} is a chelating moiety selected from the group consisting of: (CM-1), (CM-2) (CM-4) and (CM-5); or a radionuclide complex thereof.

In some embodiments, R^{a} is

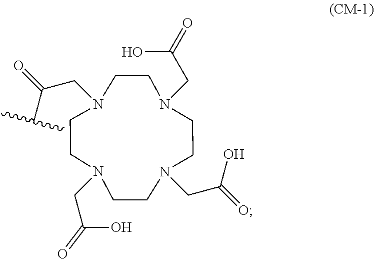 [SMILES: *C(=O)CN1CCN(CC(=O)O)CCN(CC(=O)O)CCN(CC(=O)O)CC1]

or a radionuclide complex thereof.

In some embodiments, R^{a} is: (CM-2), (CM-3), (CM-4), or (CM-5); or a radionuclide complex thereof.

In some embodiments, R^{a} is (CM-2), (CM-4), or (CM-5); or a radionuclide complex thereof.

In some embodiments, R^{a} is: (CM-2); or a radionuclide complex thereof. In some embodiments, R^{a} is: (CM-3); or a radionuclide complex thereof. In some embodiments, R^{a} is: (CM-5); or a radionuclide complex thereof.

In some embodiments, R^{a} is:

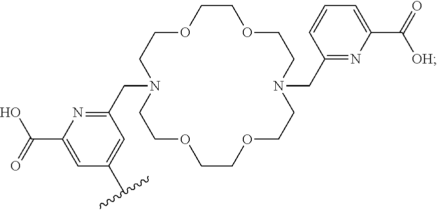 [SMILES: *c1cc(CN2CCOCCOCCN(Cc3cccc(C(=O)O)n3)CCOCCOCC2)nc(C(=O)O)c1]

or a radionuclide complex thereof. In some embodiments, R^{a} is

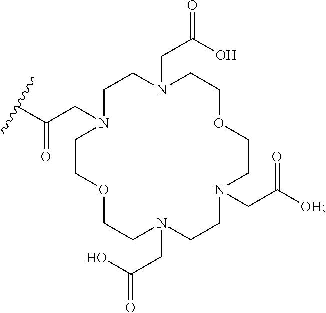 [SMILES: *C(=O)CN1CCOCCN(CC(=O)O)CCN(CC(=O)O)CCOCCN(CC(=O)O)CC1]

or a radionuclide complex thereof.

In some embodiments, R^{a} is:

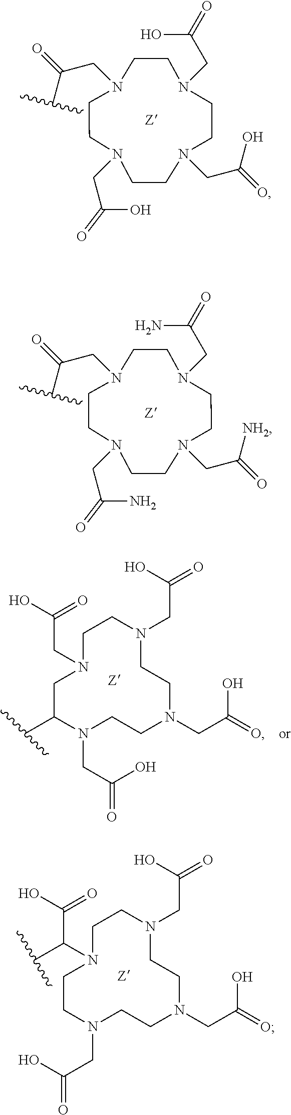

wherein Z′ is a diagnostic or therapeutic radionuclide.

In some embodiments, R^{a} is:

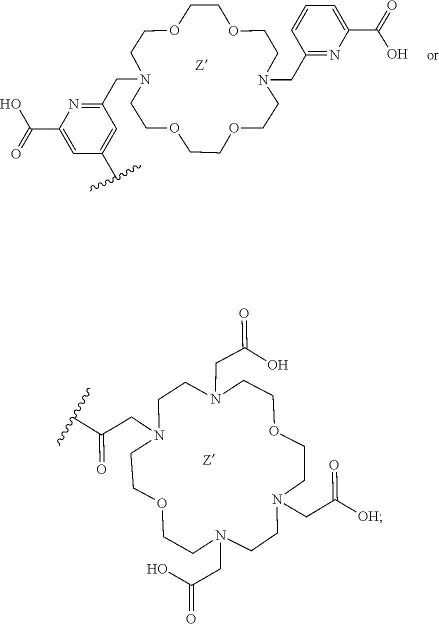

wherein Z′ is a diagnostic or therapeutic radionuclide.

In some embodiments, Z′ is an Auger electron-emitting radionuclide, α-emitting radionuclide, β-emitting radionuclide, or γ-emitting radionuclide. In some embodiments, Z′ is an Auger electron-emitting radionuclide that is 111-indium (¹¹¹In), 67-gallium (⁶⁷Ga), 68-gallium (⁶⁸Ga), 99m-technetium (^{99m}Tc), 64-copper (⁶⁴Cu), or 195m-platinum (^{195m}Pt).

In some embodiments, Z′ is an α-emitting radionuclide that is 225-actinium (²²⁵Ac), 213-bismuth (²¹³Bi), 223-Radium (²²³Ra), or 212-lead (²¹²Pb). In some embodiments, Z′ is a β-emitting radionuclide that is 90-yttrium (⁹⁰Y), 177-lutetium (¹⁷⁷Lu), iodine-131I (¹³¹I), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn).

In some embodiments, Z′ is a γ-emitting radionuclide that is 60-cobalt (⁶⁰Co), 103-palladium (¹⁰³Pd), 137-cesium (¹³⁷Cs), 169-ytterbium (169Yb), 192-iridium (¹⁹²Ir), or 226-radium (²²⁶Ra).

In some embodiments, R^{a} comprises a radionuclide (Z) and a chelator configured to bind the radionuclide (Z), wherein the radionuclide is suitable for positron emission tomography (PET) analysis, single-photon emission computerized tomography (SPECT), or magnetic resonance imaging (MRI). In some embodiments, the radionuclide is copper-64 (⁶⁴Cu), gallium-68 (⁶⁸Ga), 111-indium (¹¹¹In), or technetium-99m (^{99m}Tc).

### Metals (Radionuclides)

In some embodiments, Z′ is an Auger electron-emitting radionuclide. In some embodiments, Z′ is an α-emitting radionuclide. In some embodiments, V is a β-emitting radionuclide. In some embodiments, Z′ is a γ-emitting radionuclide. In some embodiments, the type of radionuclide used in a peptide targeted therapeutic compound can be tailored to the specific type of cancer, the type of targeting moiety (e.g., peptide ligand), etc. Radionuclides that undergo α-decay emit α-particles (helium ions with a +2 charge) from their nuclei. As a result of α-decay the daughter nuclide has 2 protons less and 2 neutrons less than the parent nuclide. This means that in α-decay, the proton number is reduced by 2 while the nucleon number is reduced by 4. Radionuclides that undergo β-decay emit β-particles (electrons) from their nuclei. During β-decay, one of the neutrons changes into a proton and an electron. The proton remains in the nucleus while the electron is emitted as a β-particle. This means that in β-decay, the nucleus loses a neutron but gains a proton. In γ-decay, a nucleus in an excited state (higher energy state) emits a γ-ray photon to change to a lower energy state. There is no change in the proton number and nucleon number during the γ-decay. The emission of γ-rays often accompanies the emission of α-particles and β-particles.

Auger electrons (AEs) are very low energy electrons that are emitted by radionuclides that decay by electron capture (EC) (e.g. ¹¹¹In, ⁶⁷Ga, ^{99m}Tc, ^{193m}pt ¹²⁵I and ¹²³I). This energy is deposited over nanometer-micrometer distances, resulting in high linear energy transfer that is potent for causing lethal damage in cancer cells. Thus, AE-emitting radiotherapeutic agents have great potential for treatment of cancer.

β-Particles are electrons emitted from the nucleus. They typically have a longer range in tissue (of the order of 1-5 mm) and are the most frequently used.

α-Particles are helium nuclei (two protons and two neutrons) that are emitted from the nucleus of a radioactive atom. Depending on their emission energy, they can travel 50-100 μm in tissue. They are positively charged and are orders of magnitude larger than electrons. The amount of energy deposited per path length travelled (designated ‘linear energy transfer’) of α-particles is approximately 400 times greater than that of electrons. This leads to substantially more damage along their path than that caused by electrons. An α-particle track leads to a preponderance of complex and largely irreparable DNA double-strand breaks. The absorbed dose required to achieve cytotoxicity relates to the number of α-particles traversing the cell nucleus. With use of this as a measure, cytotoxicity may be achieved with a range of 1 to 20 α-particle traversals of the cell nucleus. The resulting high potency, combined with the short range of α-particles (which reduces normal organ toxicity), has led to substantial interest in developing α-particle-emitting agents. The α-particle emitters typically used include bismuth-212, lead-212, bismuth-213, actinium-225, radium-223 and thorium-227.

In some embodiments, Z′ is a diagnostic or therapeutic radionuclide.

Representative Radionuclides

| | Radionuclide | |
|---|---|---|
| Isotope | t_{1/2} (h) | Decay mode |
| ⁶⁰Cu | 0.4 | β+ (93%), EC (7%) |
| ⁶¹Cu | 3.3 | β+ (62%), EC (38%) |
| ⁶²Cu | 0.16 | β+ (98%), EC (2%) |
| ⁶⁴Cu | 12.7 | β+ (19%), EC (41%), β− (40%) |
| ⁶⁷Cu | 61.9 | |
| ⁶⁶Ga | 9.5 | β+ (56%), EC (44%) |
| ⁶⁷Ga | 78.2 | EC (100%) |
| ⁶⁸Ga | 1.1 | β+ (90%), EC (10%) |
| ⁴⁴Sc | 3.9 | β+ (94%), EC (6%) |
| ⁴⁷Sc | 80.2 | β− (100%) |
| ¹¹¹In | 67.2 | EC (100%) |
| ^{114m}In | 49.5 d | EC (100%) |
| ¹¹⁴In (daughter) | 73 s | β− (100%) |
| ¹⁷⁷Lu | 159.4 | β− (100%) |
| ⁸⁶Y | 14.7 | β+ (33%), EC (66%) |
| ⁹⁰Y | 64.1 | β− (100%) |
| ⁸⁹Zr | 78.5 | β+ (23%), EC (77%) |
| ²¹²Bi | 1.1 | α (36%), β− (64%) |
| ²¹³Bi | 0.76 | α (2.2%), β− (97.8%) |
| ²¹²Pb (daughter is ²¹²Bi) | 10.6 | β− (100%) |
| ²²⁵Ac | 240 | α (100%) |
| ²²⁷Th | 448.8 | α |
| ²¹¹At | 7.2 | α |

In some embodiments, is an Ager electro-emitting radionuclide. In some embodiments, Z′ is an Auger electron-emitting radionuclide that is 111-indium (¹¹¹In), 67-gallium (⁶⁷Ga), 68-gallium (⁶⁸Ga), 99m-technetium (⁹⁹-Tc), or 195m-platinum (^{195m}Pt).

In some embodiments, Z′ is a β-emitting radionuclide. In some embodiments, Z′ is a β-emitting radionuclide that is 225-actinium (²²⁵Ac), 213-bismuth (²¹³Bi), 223-Radium (²²³Ra), or 212-lead (²¹²Pb).

In some embodiments, Z′ is a β-emitting radionuclide. In some embodiments, Z′ is a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cut) 153-samarium (¹⁵³St), 89-strontium (⁸⁹Sr) 198-gold (¹⁹⁸Au), 169-Erbium (¹⁶⁹Er), 165-dysprosium (¹⁶⁵Dy), 99m-technetium (^{99m}-Tc), 89-zirconium (¹⁹Zr), or 52-manganese (⁵²Mn).

In some embodiments, Z is a γ-emitting radionuclide. In some embodiments, Z′ is a γ-emitting radionuclide that is 60-cobalt (⁶⁰Co), 103-pallidum (¹⁰³Pd), 137-cesium (¹³⁷C), 169-ytterbium (169Yb), 192-iridium (¹⁹²Ir), or 226-radium (226Ra).

In some embodiments, Z′ is an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{195m}Pt); or Z′ is an α-emitting radionuclide that is 225-actinium (²²⁵Ac), 213-bismuth (²¹³Bi), 223-Radium (²²³Ra), or 212-lead (²¹²Pb); or Z′ is α-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (⁵³Sm), 89-strontium (⁸⁹Sr), 198-gold (¹⁹⁸Au), 169-Erbium (¹⁶⁹Er), 165-dysprosium (¹⁶⁵Dy) 99m-technetium (^{99m}Tc), 89-zirconium (⁸⁹Zr), or 52-manganese (⁵²Mn); or Z′ is a 7-emitting radionuclide that is 60-cobalt (60Co), 103-pallidum (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z′ is 90-yttrium (⁹⁰Y), 177-lutetium (¹⁷⁷Lu), 186-rhenium (¹⁸⁶Re), 188-rhenium (¹⁸⁸Re), 67-copper (⁶⁷Cu), 153-samarium (¹⁵³Sm), 89-strontium (⁸⁹Sr), 198-gold (198Au), 169-Erbium (¹⁶⁹Er), 165-dysprosium (165Dy), or technetium-99m (^{99m}Tc).

In some embodiments, Z′ is ⁹⁴Tc, ⁹⁰In, ¹¹¹In, ⁶⁷Ga, ⁶⁸Ga, ⁸⁶Y, ⁹⁰Y, ¹⁷⁷Lu, ¹⁶¹Tb, ¹⁸⁶Re, ¹⁸⁸Re, ⁶⁴Cu, ⁶⁷Cu, ⁵⁵Co, ⁵⁷Co, ⁴³Sc, ⁴⁴Sc, ⁴⁷Sc, ²²⁵Ac, ¹¹³Bi, ²¹²Bi ²¹²Pb, ²²⁷Th, ¹⁵³Sm, ¹⁶⁶Ho, ¹⁵²Gd, ¹⁵³Gd, ¹⁵⁷Gd, and ¹⁶⁶Dy.

In some embodiments, Z′ is ⁶⁷Cu, ⁶⁴Cu, ⁹⁰Y, ¹⁰⁹Pd, ¹¹¹Ag, ¹⁴⁹Pm, ¹⁵³Sm, ¹⁶⁶Ho, ^{99m}Tc, ⁶⁷Ga, ⁶⁸Ga, ¹¹¹In, ⁹⁰Y, 177Lu, ¹⁸⁶Re, ¹⁸⁸Re, ¹⁹⁷Au, ¹⁹⁸Au, ¹⁹⁹Au, ¹⁰⁵Rh, ¹⁶⁵Ho, ¹⁶¹Tb, ¹⁴⁹Pm, ⁴⁴Sc, ⁴⁷Sc, ⁷⁰As, ⁷¹As, ⁷²As, ⁷³As, ⁷⁴As, ⁷⁶As, ⁷⁷As, ²¹²Pb, ²¹²Bi, ²¹³Bi, ²²⁵Ac, ^{117m}Sn, ⁶⁷Ga, ²⁰¹TI, ¹⁶⁰Gd, ¹⁴⁸Nd, and ⁸⁹Sr.

In some embodiments, Z′ is ⁶⁸Ga, ⁴³Sc, ⁴⁴Sc, 47Sc, 177Lu, ¹⁶¹Tb, ²²⁵Ac, ²¹³Bi, ²¹²Bi, or ²¹²Pb. In some embodiments, Z′ is ⁶⁷Ga, ^{99m}Tc, ¹¹¹In, or ²⁰¹TI.

In some embodiments, the radionuclide (Z′) is ⁴⁴Sc, ⁶⁴Cu, ⁶⁷Ga, ⁶⁸Ga, ⁸⁶Y, ⁸⁹Zr, ^{99m}Tc, ¹¹¹In, or ¹⁷⁷Lu.

In some embodiments, Z′ is ⁴⁴Sc, ⁶⁴Cu, ⁶⁷Ga, ⁸⁶Y or ⁸⁹Zr. In some embodiments, Z′ is ⁶⁷Ga, ^{99m}Tc, ¹¹¹In, ¹⁷⁷Lu.

In some embodiments, Z′ is ⁶⁷Cu, ⁹⁰Y, ¹¹¹In, ¹⁷⁷Lu, ²²⁵Ac, ²¹²Pb, or ²¹³Bi.

In some embodiments, Z is 111-indium (¹¹¹In), 115-indium (¹¹¹In), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵CU), or 67-copper (⁶⁷Cu).

In some embodiments, Z′ is 111-indium (¹¹¹In). In some embodiments, Z′ is 115-indium (¹¹⁵In). In some embodiments, Z′ is 67-gallium (⁶⁷Ga). In some embodiments, Z′ is 68-gallium (⁶⁸Ga). In some embodiments, Z′ is 69-gallium (⁶⁹Ga), 71-gallium (⁷¹Ga), or a mixture thereof. In some embodiments, Z′ is 225-actinium (²²⁵Ac). In some embodiments, Z is 175-lutetium (¹⁷⁵Lu). In some embodiments, Z′ is 177-lutetium (¹⁷⁷Lu). In some embodiments, Z′ is 204-lead (²⁰⁴Pb), 206-lead (²⁰⁶Pb), 207-lead (²⁰⁷Pb) 208-lead (²⁰⁸Pb), or a mixture thereof. In some embodiments, Z is 212-lead (²¹²Pb). In some embodiments, Z′ is 64-copper (⁶⁴Cu). In some embodiments, Z′ is 63-copper (⁶³Cu), 65-copper (⁶⁵Cu), or a mixture thereof. In some embodiments, Z is 67-copper (⁶⁷Cu).

In some embodiments, Z′ is 111-indium (¹¹¹In), 115-indium (¹¹⁵In), 67-gallium (⁶⁷Ga), 68-gallium (⁶⁸Ga), 225-actinium (²²⁵Ac), 175-lutetium (¹⁷⁵Lu) or 177-lutetium (¹⁷⁷Lu).

### Exemplary Chelator and Radionuclide Complexes

Radionuclides have useful emission properties that can be used for diagnostic imaging techniques, such as single photon emission computed tomography (SPECT, e.g. ⁶⁷Ga, ^{99m}Tc, ¹¹¹In, ¹⁷⁷Lu) and positron emission tomography (PET, e.g. ⁶⁸Ga, ⁶⁶Cu, ⁴⁴Sc, ⁸⁶Y, ⁸⁹Zr), as well as therapeutic applications (e.g. ⁴⁷Sc, ¹¹⁴mIn, ¹⁷⁷Lu, ⁹⁰Y, ^{212/213}Bi, ²¹²Pb, ²²⁵Ac, ^{186/188}Re). A fundamental component of a radiometal-based radiopharmaceutical is the chelator, the ligand system that binds the radiometal ion in a tight stable coordination complex so that it can be properly directed to a desirable molecular target in vivo. Guidance for selecting the optimal match between chelator and radiometal for a particular use is provided in the art (e.g., see Price et al., “Matching chelators to radiometals for radiopharmaceuticals”, Chem. Soc. Rev., 2014, 43, 260-290).

In some embodiments, R^{a} is a chelating moiety selected from the group consisting of: DOTA; DO3A; DO2A; DOTMA; DOTAM; DOTPA; Bn-DOTA; p-OH-Bn-DOTA; H₄pypa; H₄pypa-benzyl; H₄py4pa; H₄py4pa-benzyl; H₄octapa; H₄octapa-benzyl; and TTHA; or a radionuclide complex thereof.

In some embodiments, R^{a} is:

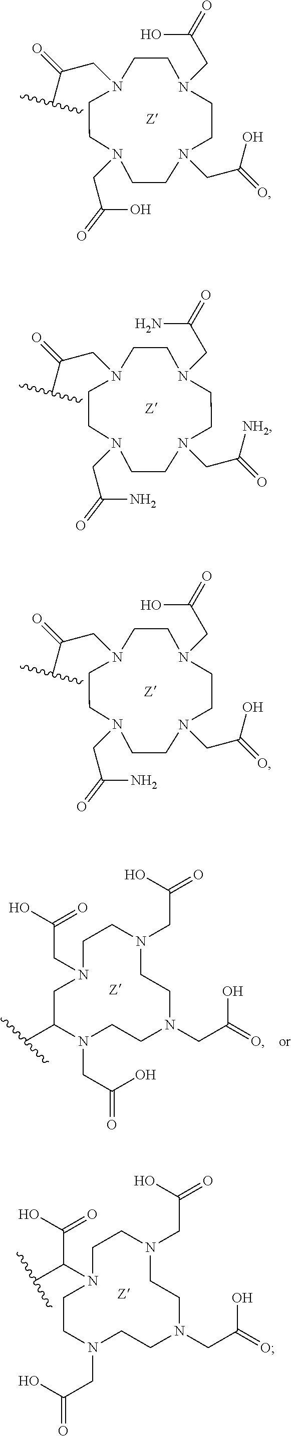

wherein Z′ is a diagnostic or therapeutic radionuclide.

In some embodiments, the radionuclide (Z′) is ⁴⁴Sc, ⁶⁴Cu, ⁶⁷Ga, ⁶⁸Ga, ⁸⁶Y, ⁸⁹Zr, ^{99m}Tc, ¹¹¹In, or ¹⁷⁷Lu. In some embodiments, the radionuclide (Z′) is ⁴⁴Sc, ⁶⁴Cu, ⁶⁸Ga, ⁸⁶Y, or ⁸⁹Zr. In some embodiments, the radionuclide (Z′) is ⁶⁷Ga, ^{99m}Tc, ¹¹¹In, or ¹⁷⁷Lu.

In some embodiments, the radionuclide (Z′) is ⁶⁷Cu, ⁹⁰Y, ¹¹¹In, ¹⁷⁷Lu, ²²⁵Ac, ²¹²Pb, or ²¹³Bi.

In some embodiments, the radionuclide (Z′) is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 225-actinium (²²⁵Ac)., 175-lutetium (¹⁷⁵Lu), 177-lutetium (¹⁷⁷Lu), 206-lead (²⁰⁶Pb), 207-lead (²⁰⁷Pb), 208-lead (²⁰⁸Pb), 212-lead (²¹²Pb), 60-copper (⁶⁰Cu), 61-copper (61Cu), 62-copper (⁶¹Cu), 63-copper (⁶³Cu), 64-copper (⁶⁴Cu), 65-copper (⁶⁵Cu), or 67-copper (⁶⁷Cu).

In some embodiments, the radionuclide (Z′) is ¹¹¹-indium (¹¹¹In) or 115-indium (¹¹⁵In), or a mixture thereof. In some embodiments, the radionuclide (Z′) is 67-gallium (⁶⁷Ga), 68-gallium (⁶⁸Ga), 69-gallium (⁶⁹Ga), or 71-gallium (71Ga), or a mixture thereof. In some embodiments, the radionuclide (Z′) is 225-actinium (²²⁵Ac). In some embodiments, the radionuclide (Z′) is 175-lutetium (175Lu) or 17⁷-lutetium (177Lu), or a mixture thereof. In some embodiments, the radionuclide (Z′) is 206-lead (²⁰⁶Pb), 207-lead (²⁰⁷Pb), 208-lead (²⁰⁸Pb), or 212-lead (²¹²Pb), or a mixture thereof. In some embodiments, the radionuclide (Z) is 60-copper (⁶⁰Cu), 61-copper (⁶¹Cu), 62-copper (⁶²Cu), 63-copper (⁶³Cu), 64-copper (⁶⁴Cu), 65-copper (⁶⁵Cu), or 67-copper (⁶⁷Cu), or a mixture thereof.

In some embodiments, the radionuclide (Z′) is 111-indium (¹¹¹In), 115-indium (¹¹⁵In), 67-gallium (⁶⁷Ga), 68-gallium (⁶⁸Ga), 225-actinium (²²⁵Ac), 175-lutetium (¹⁷⁵Lu) or 177-lutetium (¹⁷⁷Lu).

In some embodiments, the radionuclide (Z) is 90-yttrium (⁹⁰Y), 177-lutetium (¹⁷⁷Lu), 186-rhenium (¹⁸⁶Re), 188-rhenium (¹⁸⁸Re), 67-copper (67Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), or technetium-99m (^{99m}Tc)

### Emission Tomography

In some embodiments, R^{a} comprises a chelated radionuclide that is suitable for positron emission tomography (PET) analysis or single-photon emission computerized tomography (SPECT). In some embodiments, R^{a} comprises a chelated radionuclide that is suitable for single-photon emission computerized tomography (SPECT). In some embodiments, R^{a} comprises a chelated radionuclide that is suitable for positron emission tomography (PET) analysis. In some embodiments, R, comprises a chelated radionuclide that is suitable for positron emission tomography imaging, positron emission tomography with computed tomography imaging, or positron emission tomography with magnetic resonance imaging (MRI).

In some embodiments, R^{a} is a chelating moiety selected from the group consisting of: DOTA; DO3A; DO2A; DOTMA; DOTAM; DOTPA; En-DOTA; p-OH-Bn-DOTA; H₄pypa; H₄pypa-benzyl; H₄py4pa; H₄py4pa-benzyl; H₄octapa; H₄octapa-benzyl; and TTHA; or a radionuclide complex thereof. In some embodiments, the radionuclide is copper-64 (⁶⁴Cu, gallium-68 (⁶⁸Ga), or technetium-99m (^{99m}Tc).

In some embodiments, a conjugate described herein is designed to have a prescribed elimination profile. The elimination profile can be designed by adjusting the sequence and length of the peptide ligand, the property of the linker, the type of radionuclide, etc. In some embodiments, the conjugate has an elimination half-life of about 5 minutes to about 12 hours. In some embodiments, the conjugate has an elimination half-life of about 10 minutes to about 8 hours. In some embodiments, the conjugate has an elimination half-life of at least about 15 minutes, at least about 30 minutes, at least about 1 hour, at least about 2 hours, at least about 3 hours, at least about 4 hours, at least about 5 hours, at least about 6 hours, or at least about 8 hours. In some embodiments, the conjugate has an elimination half-life of at most about 15 minutes, at most about 30 minutes, at most about 1 hour, at most about 2 hours, at most about 3 hours, at most about 4 hours, at most about 5 hours, at most about 6 hours, or at most about 8 hours. In some embodiments, the elimination half-life is determined in rats. In some embodiments, the elimination half-life is determined in humans.

A herein described conjugate can have an elimination half-life in a tumor and non-tumor tissue of the subject. The elimination half-life in a tumor can be the same as or different from (either longer or shorter than) the elimination half-life in a non-tumor issue. In some embodiments, the elimination half-life of the conjugate in a tumor is about 15 minutes to about 1 day. In some embodiments, the elimination half-life of the conjugate in a tumor is at least 1.1, at least 1.2, at least 1.3, at least 1.4, at least 1.5, at least 2.0, at least 2.5, at least 3.0, at least 4.0, or at least 5.0-fold of the elimination half-life of the conjugate in a non-tumor tissue of the subject.

As used herein, the “elimination half-life” can refer to the time it takes from the maximum concentration after administration to half maximum concentration. In some embodiments, the elimination half-life is determined after intravenous administration. In some embodiments, the elimination half-life is measured as biological half-life, which is the half-life of the pharmaceutical in the living system. In some embodiments, the elimination half-life is measured as effective half-life, which is the half-life of a radiopharmaceutical in a living system taking into account the half-life of the radionuclide.

Response and toxicity prediction is essential for the rational implementation of cancer therapy. The biological effects of radionuclide therapy are mediated by a well-defined physical quantity, the absorbed dose (D), which is defined as the energy absorbed per unit mass of tissue.

Radiation dosimetry is the measurement, calculation and assessment of the ionizing radiation dose absorbed by an object, usually the human body, and may be thought of as the ability to perform the equivalent of a pharmacodynamic study in treated patients in real time. This applies both internally, due to ingested or inhaled radioactive substances, or externally due to irradiation by sources of radiation. Dosimetry analysis may be performed as part of patient treatment to calculate tumor versus normal organ absorbed dose and therefore the likelihood of treatment success.

A conjugate described herein can have a prescribed time-integrated activity coefficient (i.e., a) in a tumor or non-tumor tissues of a subject. As used herein, A represents the cumulative number of nuclear transformations occurring in a source tissue over a dose-integration period per unit administered activity. The ã value of a conjugate can be tuned by modifications of the NPDC. The ã value can be determined using a method known in the art. In some embodiments, the ãvalue of the conjugate in a tumor is from about 10 minutes to about 1 day. The ã value of the conjugate in a tumor can be the same as the ãvalue of the conjugate in a non-tumor tissue of the subject. The ã value of the conjugate in a tumor can be longer or shorter than the ã value of the conjugate in a non-tumor tissue of the subject. In some embodiments, the ã value of the conjugate in a tumor is at least 1.1, at least 1.2, at least 1.3, at least 1.4, at least 1.5, at least 20, at least 2.5, at least 3.0, at least 4.0, or at least 5.0-fold of the ã value of the conjugate in a non-tumor tissue of the subject.

A conjugate described herein can have an \tilde{α} value in an organ of a subject. In some embodiments, the conjugate has an \tilde{α} value in a kidney of the subject of at most 24 hours. In some embodiments, the ã value of the conjugate in a kidney of the subject is at most 18 hours, 15 hours, 12 hours, 10 hours, 8 hours, 6 hours, or 5 hours. In some embodiments, the ã value of the conjugate in a kidney of the subject is about 30 minutes to about 24 hours. In some embodiments, the ã value of the conjugate in a kidney of the subject is about 2 to 24 hours. In some embodiments, the ã value of the conjugate in a kidney of the subject is more than 24 hours. In some embodiments, the ã value of the conjugate in a liver of the subject is at most 24 hours. In some embodiments, the ã value of the conjugate in a liver of the subject is at most 18 hours, 15 hours, 12 hours, 10 hours, 8 hours, 6 hours, or 5 hours. In some embodiments, the ã value of the conjugate in a liver of the subject is about 30 minutes to about 24 hours. In some embodiments, the ã value of the conjugate in a liver of the subject is about 2 to 24 hours. In some embodiments, the ã value of the conjugate in a liver of the subject is more than 24 hours.

### Linkers

In some embodiments, the linker has a prescribed length thereby linking the Kisspeptin receptor (KISS1R) targeting ligand and the chelating moiety or a radionuclide complex thereof (R^{a}) while allowing an appropriate distance therebetween.

For the linkers described herein, no orientation of the linker is implied by the direction in which the formula of the linker is written. For example, the formula

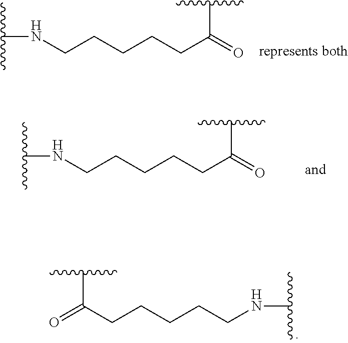 [SMILES: *NCCCCCC(*)=O ; *NCCCCCC(*)=O ; *NCCCCCC(*)=O]

Additionally, the formula

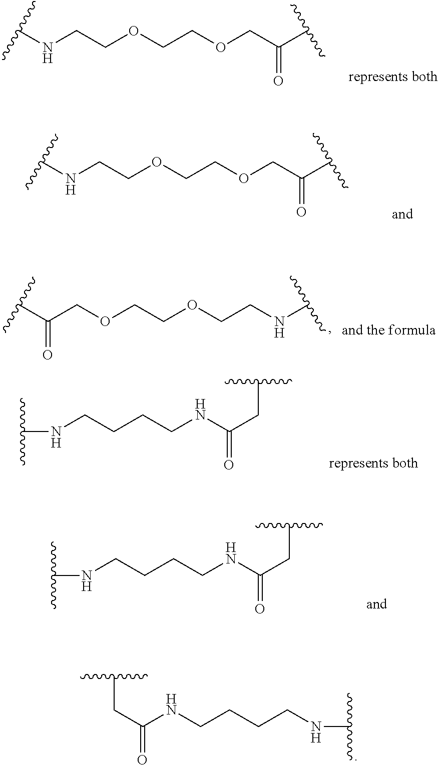 [SMILES: *NCCOCCOCC(*)=O ; *NCCOCCOCC(*)=O ; *NCCOCCOCC(*)=O ; *CC(=O)NCCCCN* ; *CC(=O)NCCCCN* ; *CC(=O)NCCCCN*]

In some embodiments, the linker is flexible. In some embodiments, the linker is rigid.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, the linker comprises one or more linear structures, one or more non-linear structures, one or more branched structures, one or more cyclic structures, one or more flexible moieties, one or more rigid moieties, or combinations thereof.

In some embodiments, a linker comprises one or more amino acid residues. In some embodiments, the linker comprises 1 to 3, 1 to 5, 1 to 10, 5 to 10, or 5 to 20 amino acid residues. In some embodiments, one or more amino acids of the linker are unnatural amino acids.

In some embodiments, the linker comprises a peptide linkage. The peptide linkage comprises L-amino acids and/or D-amino acids. In some embodiments, D-amino acids are preferred in order to minimize immunogenicity and nonspecific cleavage by background peptidases or proteases.

In some embodiments, a linker has 1 to 100 atoms, 1 to 50 atoms, 1 to 30 atoms, 1 to 20 atoms, 1 to 15 atoms, 1 to 10 atoms, or 1 to 5 atoms in length. In some embodiments, the linker has 1 to 10 atoms in length. In some embodiments, the linker has 1 to 20 atoms in length.

In some embodiments, a linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences, proline-rich sequences, and regions rich in double and/or triple bonds.

In some embodiments, the linker comprises a click chemistry residue. In some embodiments, the linker is attached to a peptide ligand, to a metal chelator or both via click chemistry. For example, in some embodiments, a peptide ligand comprises an azide group that reacts with an alkyne moiety of the linker. For another example, in some embodiments, a peptide ligand comprises an alkyne group that reacts with an azide of the linker. The metal chelator and the linker can be attached similarly. In some embodiments, the linker comprises an azide moiety, an alkyne moiety, or both. In some embodiments, the linker comprises a triazole moiety.

In some embodiments,
-L- is absent, *-L, *—NR¹⁷-L¹-, *—NR¹⁷-L⁵-L¹, *—NR¹⁷-L⁵-C(═O)-L¹, *—NR¹⁷-L⁵-NR¹⁷—C(═O)-L¹-, *-L⁵-C(═O)-L¹-, *-L⁵-L¹, *—NR¹⁷-L⁵-NR¹⁷-L¹, *—NR¹⁷-L⁵-C(═O)NR¹⁷-L⁵,*-(L³)_{w}-, *—NR¹⁷-L⁵-C(═O)-L³-NR¹⁷-L⁵-C(═O)—, or *-(L³)_{w}-NR¹⁷-L⁵-C(═O)-L¹; wherein * denotes the attachment point to R^{a};
L⁵ is substituted or unsubstituted —C₁-C₆ alkylene; or L⁵ and R¹⁷ are taken together with the N atom to which they are attached to form N-heterocycloalkyl;
R¹⁷ is selected from hydrogen, —C₁-C₆ alkyl, —C₁-C₆ alkyl-CO₂H, —(CH₂CH₂O)_{z}—CH₂CH₂—CO₂H;
L¹ is absent, -L²-, -L²-(L³)_{w}-, -(L³)_{w}-L²-, or -L²-(L³)_{w}-L²-(L³)_{w};
each L² is independently absent, —C₀-C₆ alkylene-(substituted or unsubstituted arylene)-C₀-C₆ alkylene-C(═O)—, —C₀-C₆ alkylene-(substituted or unsubstituted arylene)-C₀-C₆ alkylene-OC(═O)—, —C₀-C₆ alkylene-(substituted or unsubstituted cyclohexylene)-C₀-C₆ alkylene-C(═O)—, —C₀-C₆ alkylene-(substituted or unsubstituted heterocycloalkylene)-C₀-C₆ alkylene-C₀-C₆)—, —C₀-C₆alkylene-(substituted or unsubstituted heteroarylene)-C₀-C₆ alkylene-C(═O)—, C₀-C₆ alkylene-(substituted or unsubstituted heteroarylene)-C₀-C₆alkylene-OC(═O)—, —C₁-C₂₀ polyethylene glycol, —C₄-C₂₀ polyethylene glycol-C(═O)—, substituted or unsubstituted C₁-C₂₀ alkylene, substituted or un substituted C₁-C₂₀, alkylene-C(═O)—, substituted or unsubstituted 2 to 20 membered heteroalkylene, —(CH₂CH₂O)_{z}—CH₂—, —(CH₂CH₂O)_{z}—CH₂CH₂—, —(CH₂CH₂O)_{z}—CH₂—C(═O)—; or —(CH₂CH₂O)_{z}—CH₂CH₂—C(═O)—;
each z is independently 1, 2, 3,4, 5, or 6;
each L³ is independently selected from natural or unnatural amino acids, wherein any free amine of an amino acid or peptide bond is optionally independently substituted with L-, and wherein when two or more amino acids are present then the N atom of the amide linking the amino acids is optionally substituted with —CH₃;
each L⁴ is independently selected from C₁-C₅ alkylene, C(═O)—C₁-C₆ akylene-C(═O), C(═O)—NH—C₁-C₆ alkylene-C(═O), C(═O)—C₁-C₆ alkylene-(substituted or unsubstituted heteroarylene)-C₁-C₆ alkylene-C(═O), C₁-C₆ alkylene-C(═O), —C(═O)—C₁-C₆ alkylene-(substituted or unsubstituted arylene), and C₁-C₆ alkylene-(substituted or unsubstituted arylene)-C(═O); wherein if L⁴ is present then: L⁴ is attached to the any one of X¹, X², X³, X⁴, X⁵, X⁶, or X⁷, or L⁴ is attached to X⁸ if X, X², X³, X⁴, X⁵, X⁶, and X⁷ are absent;
each w is independently 1, 2, 3, 4, 5, or 6.

In some embodiments, each L² is independently absent, -(substituted or unsubstituted phenylene)-C₀-C₆ alkylene-C(═O)—, -(substituted or unsubstituted cyclohexylene)-C₀-C₆ alkylene-C(═O)—, -(substituted or unsubstituted heterocycloalkylene)-C₀-C₆ alkylene-C(═O)—, -(substituted or unsubstituted heteroarylene)-C₀-C₆alkylene-O(═O)—, substituted or unsubstituted C₁-C₂₀ alkylene-C(═O)—, —(CH₂CH₂O)_{z}—CH₂—, —(CH₂CH₂O)_{z}—CH₂CH₂—, —(CH₂CH₂O)_{z}—CH₂—C(═O)—; or —(CH₂CH₂O)—CH₂CH₂—C(═O)—, wherein each z is independently 1, 2, 3, 4, 5, or 6.

In some embodiments, each L² is independently absent, -(substituted or unsubstituted phenylene)-C₀-C₆ alkylene-C(═O)—, -(substituted or unsubstituted cyclohexylene)-C₀-C₆ alkylene-C(═O)—, -(substituted or unsubstituted heterocycloalkylene)-C₀-C₆ alkylene-C(═O)—, -(substituted or unsubstituted heteroarylene)-C₀-C₆alkyl-C(═O)—, substituted or unsubstituted C₀-C₂₀ alkylene-C(═O)—, —(CH₂CH₂O)_{z}—CH₂—C(═O)—; or —(CH₂CH₂O)_{z}—CH₂CH₂—C(═O)—, wherein each z is independently 1, 2, 3, 4, 5, or 6.

In some embodiments, -L- is *—NR¹⁷—*—NR¹⁷—*—NR¹⁷-L⁵-C(═O)—, —NR¹⁷—C₀-C₆ alkylene-(substituted or unsubstituted phenylene)-C₀-C₆ alkylene-C(═O)—, *—NR¹⁷—C₀-C₆ alkylene-(substituted or unsubstituted cyclohexylene)-C₀-C₆ alkylene-C(═O)—, *—NR¹⁷—C₀-C₆ alkylene-(substituted or unsubstituted heterocycloalkylene)-C₀-C₆ alkylene-C(═O)—, *—NR¹⁷—C₀-C₆ alkylene-(substituted or unsubstituted heteroarylene)-C₀-C₆ alkylene-C(═O)—, *—NR¹⁷-substituted or unsubstituted C₁-C₂₀alkylene-C(═O)—, *—NR¹⁷—(CH₂CH₂O)_{z}—CH₂—C(═O)—, *—NR¹⁷—(CH₂CH₂O)_{z}—CH₂CH₂—C(═O)—, *—R¹⁷-L⁵-C(═O)-(L³)_{w}-, *—NR⁵-L⁵-C(═O)NR¹⁷—C₀-C₆ alkylene-(substituted or unsubstituted phenylene)-C₀-C₆ alkylene-C(═O)—, *—NR⁵-L⁵-C(═O)NR¹⁷—C₀-C₆ alkylene-(substituted or unsubstituted cyclohexylene)-C₀-C₆ alkylene-C(═O)—, *—NR⁵-L⁵-C(═O)NR¹⁷—C₀-C₆ alkylene-(substituted or unsubstituted heterocycloalkylene)-C₀-C₆ alkylene-C(═O)—, or *—NR⁵-L⁵-C(═O)NR¹⁷—C₀-C₆ alkylene-(substituted or unsubstituted heteroarylene)-C₀-C₆ alkylene-C(═O)—, wherein each z is independently 1, 2, 3, 4, 5, or 6, and wherein * denotes the attachment point to R^{a}

In some embodiments, each L³ is independently selected from the group consisting of alanine (Ala), arginine (Arg), asparagine (Asn), aspartate (Asp), glutamine (Gln), glutamate (Glu), glycine (Gly), leucine (Leu), lysine (Lys), 3-(2-naphthyl)-L-alanine (2-Nab), 3-4-pyridyl)alanine (4-Pal), phenylalanine (Phe), serine (Ser), sarcosine, tyrosine (Tyr), 3-sulfo-alanine (Ala-SO₃H), methionine (Met), valine (Val), 2-(3-aminopropoxy)-[1,1-biphenyl]-4-carboxylic acid, 2′-(3-aminopropoxy)-[1,1-biphenyl]-4-carboxylic acid, 0-(dihydroxy(oxo)-16-phosphaneyl)-L-serine, (S)-2-amino-4-(2-tetrazol-5-yl)butanoic acid, and (S)-2-amino-3-(anthracen-9-yl)propanoic acid, wherein any free amine of an amino acid or peptide bond is optionally independently substituted with L⁴, and wherein when two or more amino acids are present then the N atom of the amide linking the amino acids is optionally substituted with —CH₃. In some embodiments, each L³ is independently selected from the group consisting of alanine (Ala), glycine (Gly), serine (Ser), sarcosine, methionine (Met), 3-sulfo-alanine (Ala-SO₃H), and valine (Val), wherein any free amine of an amino acid or peptide bond is optionally independently substituted with L⁴, wherein L⁴ is —C(═O)—C₁-C₆ alkylene-C(═O)— or —C(═O)—NH—C₁-C₆ alkylene-C(═O)—, and wherein when two or more amino acids are present then the N atom of the amide linking the amino acids is optionally substituted with —CH₃.

In some embodiments, -(L³)_{w}- is sarcosine, sarcosine-sarcosine, sarcosine-sarcosine-sarcosine, sarcosine-sarcosine-sarcosine-sarcosine (SEQ ID NO: 24), sarcosine-sarcosine-sarcosine-sarcosine-sarcosine (SEQ ID NO: 25), sarcosine-sarcosine-sarcosine-sarcosine-sarcosine-sarcosine (SEQ ID NO: 831), valine-citrulline, valine-alanine, methionine-valine-lysine, glycine-phenylalanine-glycine-glycine (SEQ ID NO: 832), tyrosine-arginine-valine, arginine-valine, 3-sulfo-alanine, 3-sulfo-alanine-3-sulfo-alanine, 3-sulfo-alanine-3-sulfo-alanine-3-sulfo-alanine, glycine-glutamate, glycine-glutamate-glycine, glycine-glutamate-glutamate, methionine-tryptophan-lysine, methionine-phenylalanine-lysine, methionine-valine, methionine-valine-lysine, or phenylalanine-lysine.

In some embodiments, -(L³)_{w}- is sarcosine, sarcosine-sarcosine, sarcosine-sarcosine-sarcosine, sarcosine-sarcosine-sarcosine-sarcosine (SEQ ID NO: 24), sarcosine-sarcosine-sarcosine-sarcosine-sarcosine (SEQ ID NO: 25), sarcosine-sarcosine-sarcosine-sarcosine-sarcosine-sarcosine (SEQ ID NO: 831), valine-citrulline, valine-alanine, methionine-valine-lysine, glycine-phenylalanine-glycine-glycine (SEQ ID NO: 832), tyrosine-arginine-valine, arginine-valine, 3-sulfo-alanine, 3-sulfo-alanine-3-sulfo-alanine, 3-sulfo-alanine-3-sulfo-alanine-3-sulfo-alanine, glycine-glutamate, glycine-glutamate-glycine, glycine-glutamate-glutamate, methionine-tryptophan-lysine, methionine-phenylalanine-lysine, methionine-valine, methionine-valine-lysine, or phenylalanine-lysine, wherein the free amine of lysine is optionally independently substituted with L⁴; L⁴ is —C(O)—(CH₂)₃—C(═O)—, —C(═O)—CH₂)₄—C(═O)—, —C(═O)—(CH₂)₅—C(═O)—, —C(═O)—(CH₂)₆—C(═O)—, —C(═O)NH—(CH₂)₃—C(═O)—, —C(═O)NH—(CH₂)₄—C(═O)—, —C(═O)NH—(CH₂)₅—C(═O)—, —C(═O)NH—(CH₂)₆—C(═O)—, —C(═O)—(CH₂)₂-(triazolylene)-(CH₂)₁—C(═O)— or —C(═O)—(C H₂)₂-(triazolylene)-(CH₂)₂—C(═O)—; wherein if L⁴ is present then: L⁴ is attached to the any one of X¹, X², X³, X⁴, X⁵, X⁶, or X⁷, or L⁴ is attached to X if X¹, X², X³, X⁴, X⁵, X⁶, and X⁷ are absent.

In some embodiments, -L- is:

absent,

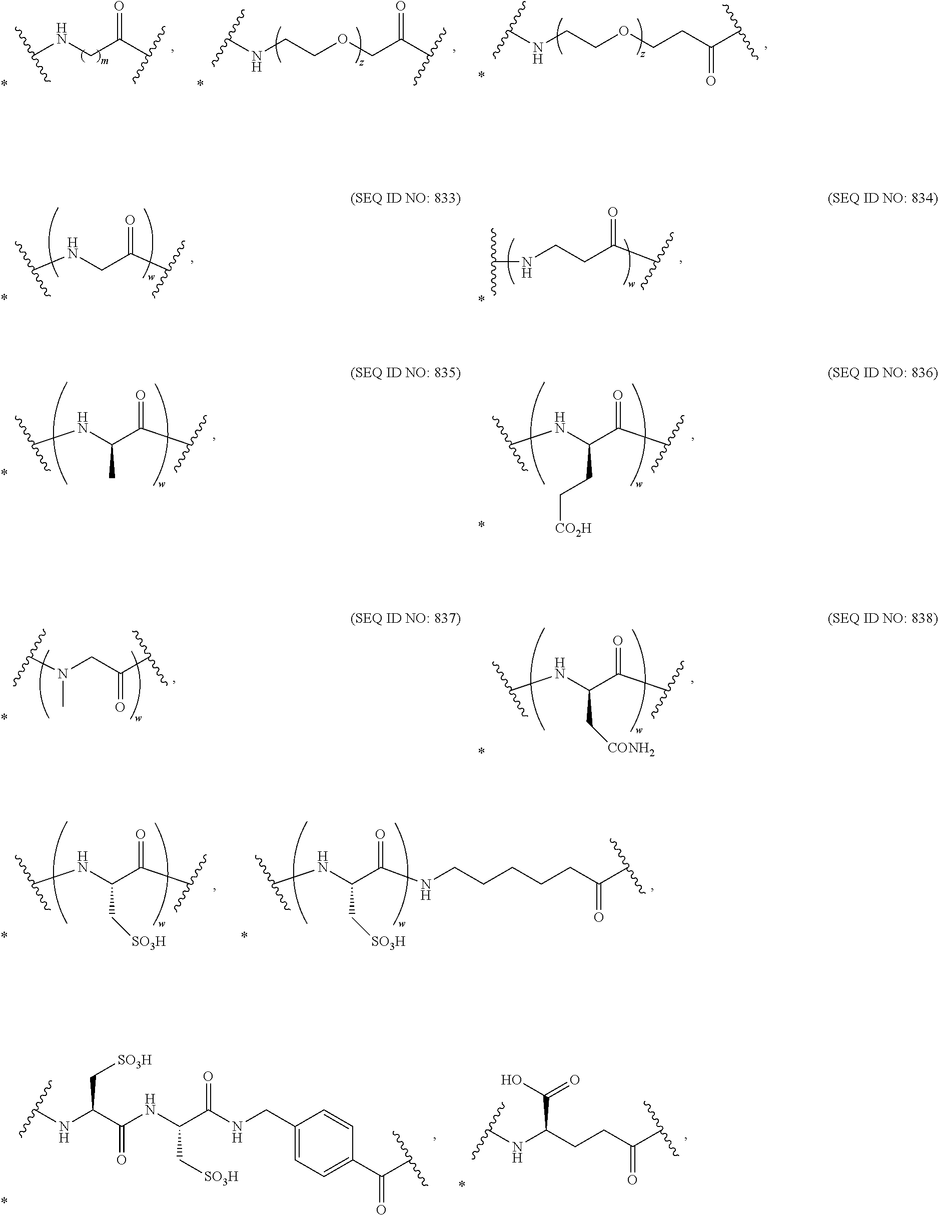

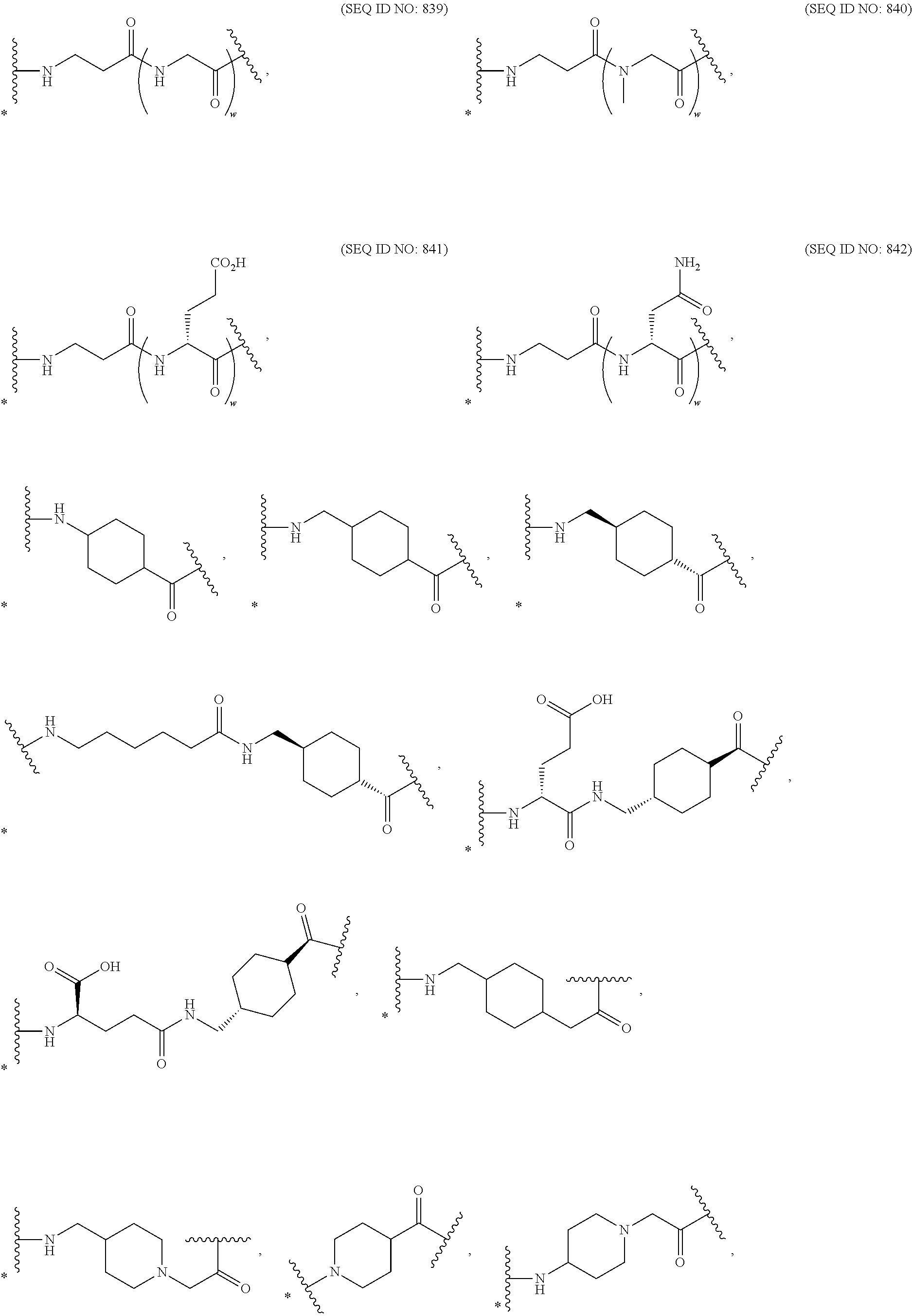

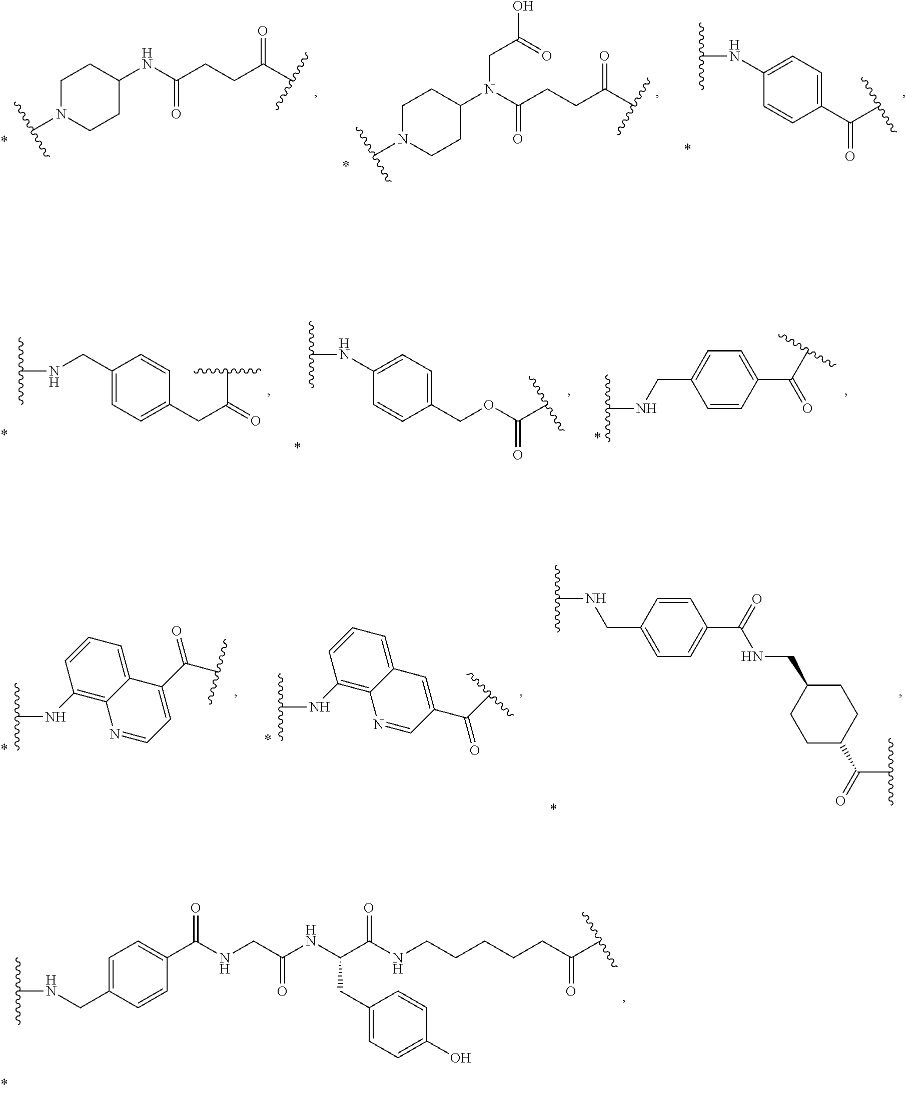

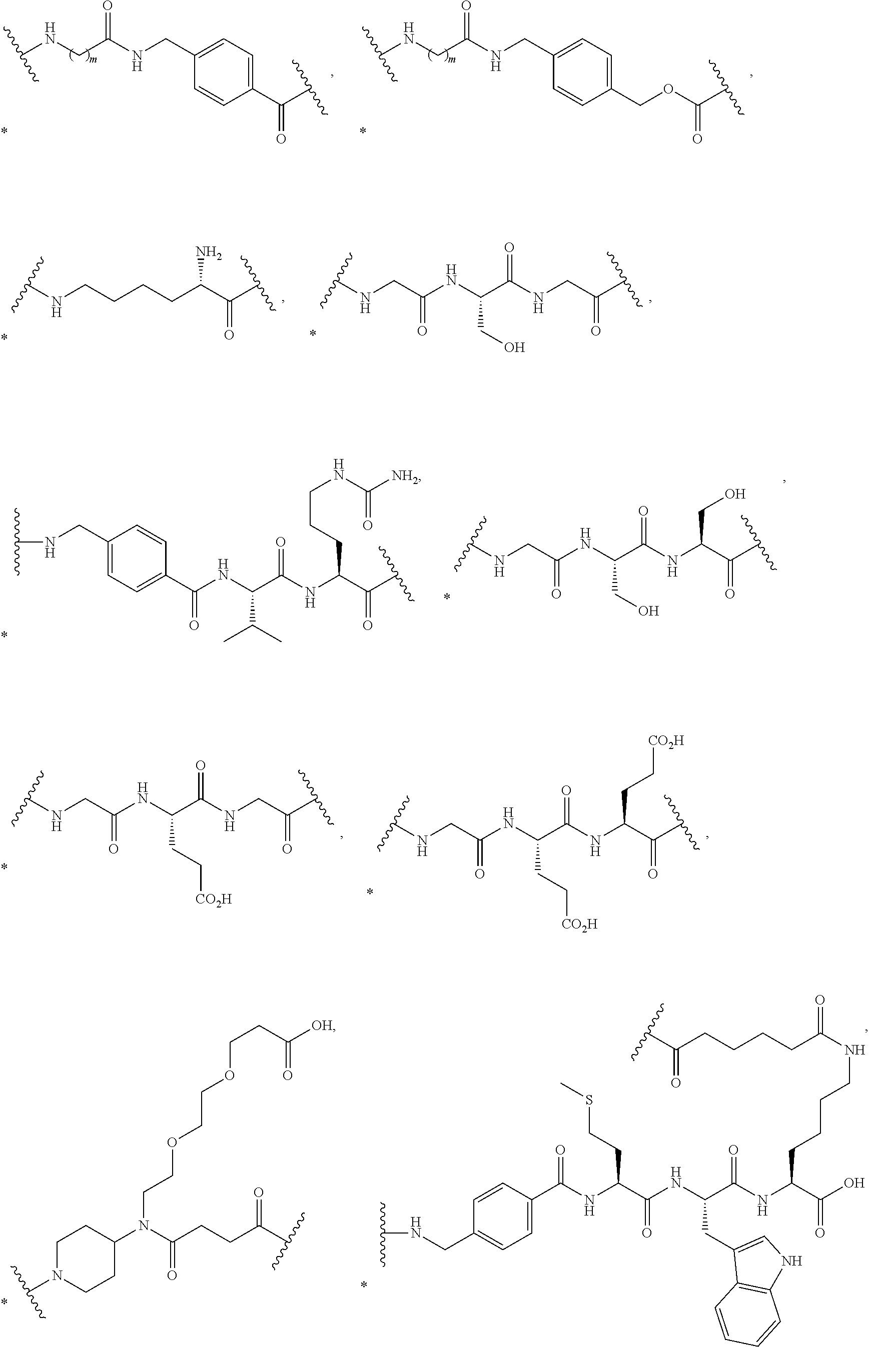

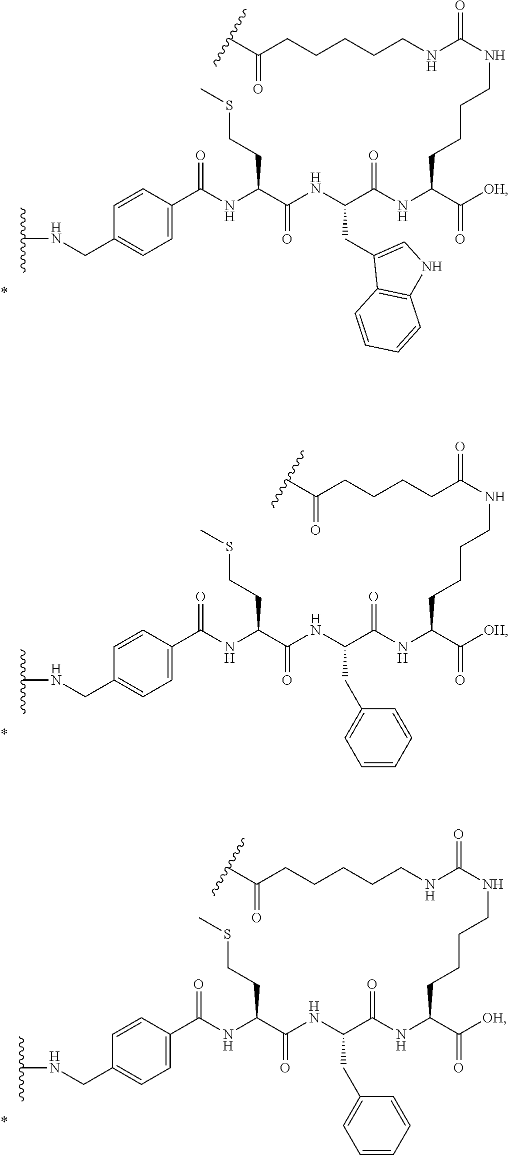 [SMILES: *NCc1ccc(C(=O)N[C@@H](CCSC)C(=O)N[C@@H](Cc2c[nH]c3ccccc23)C(=O)N[C@@H](CCCCNC(=O)NCCCCCC(*)=O)C(=O)O)cc1 ; *NCc1ccc(C(=O)N[C@@H](CCSC)C(=O)N[C@@H](Cc2ccccc2)C(=O)N[C@@H](CCCCNC(=O)CCCCC(*)=O)C(=O)O)cc1 ; *NCc1ccc(C(=O)N[C@@H](CCSC)C(=O)N[C@@H](Cc2ccccc2)C(=O)N[C@@H](CCCCNC(=O)NCCCCCC(*)=O)C(=O)O)cc1]

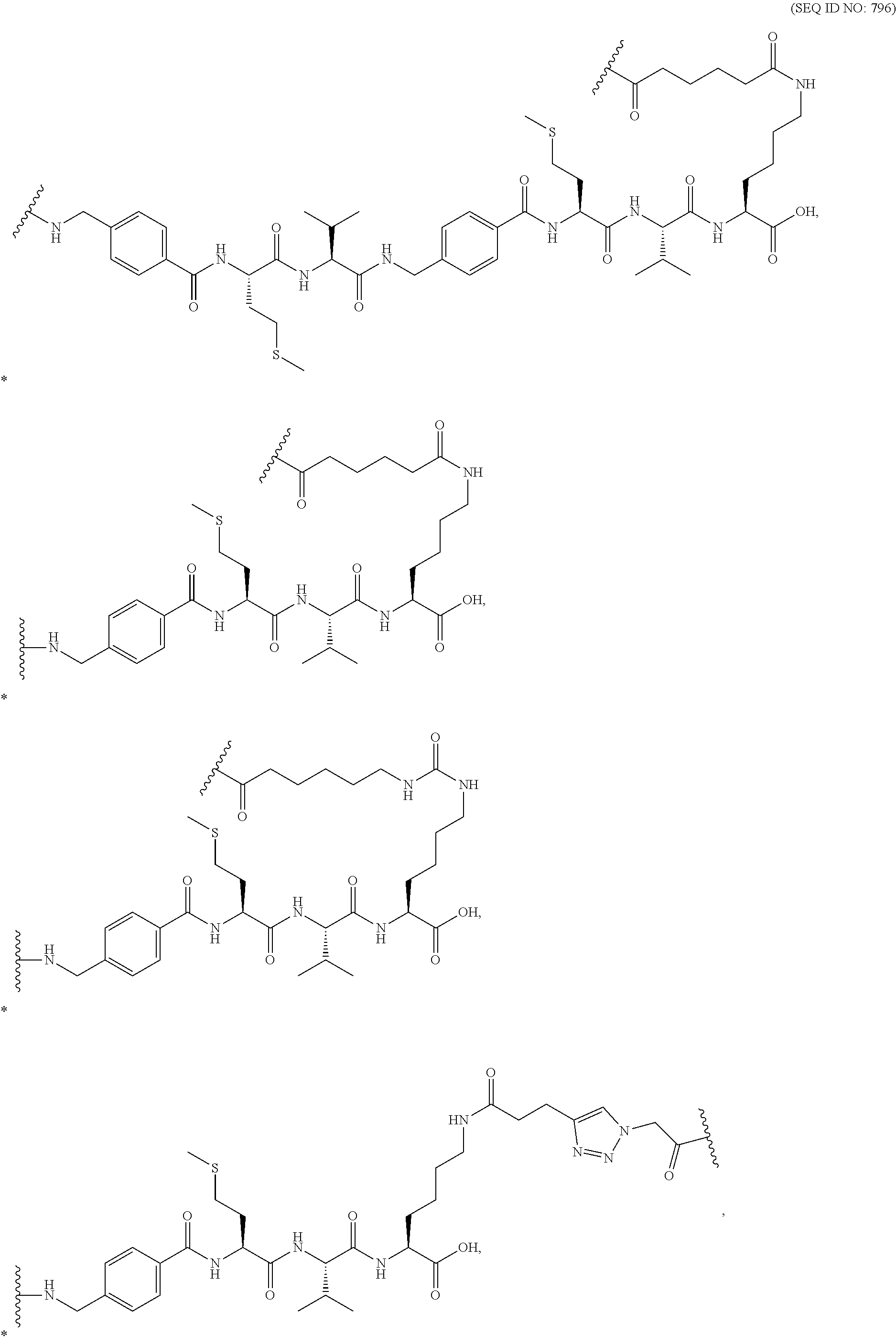 [SMILES: *NCc1ccc(C(=O)N[C@@H](CCSC)C(=O)N[C@H](C(=O)NCc2ccc(C(=O)N[C@@H](CCSC)C(=O)N[C@H](C(=O)N[C@@H](CCCCNC(=O)CCCCC(*)=O)C(=O)O)C(C)C)cc2)C(C)C)cc1 ; *NCc1ccc(C(=O)N[C@@H](CCSC)C(=O)N[C@H](C(=O)N[C@@H](CCCCNC(=O)CCCCC(*)=O)C(=O)O)C(C)C)cc1 ; *NCc1ccc(C(=O)N[C@@H](CCSC)C(=O)N[C@H](C(=O)N[C@@H](CCCCNC(=O)NCCCCCC(*)=O)C(=O)O)C(C)C)cc1 ; *NCc1ccc(C(=O)N[C@@H](CCSC)C(=O)N[C@H](C(=O)N[C@@H](CCCCNC(=O)CCc2cn(CC(*)=O)nn2)C(=O)O)C(C)C)cc1]

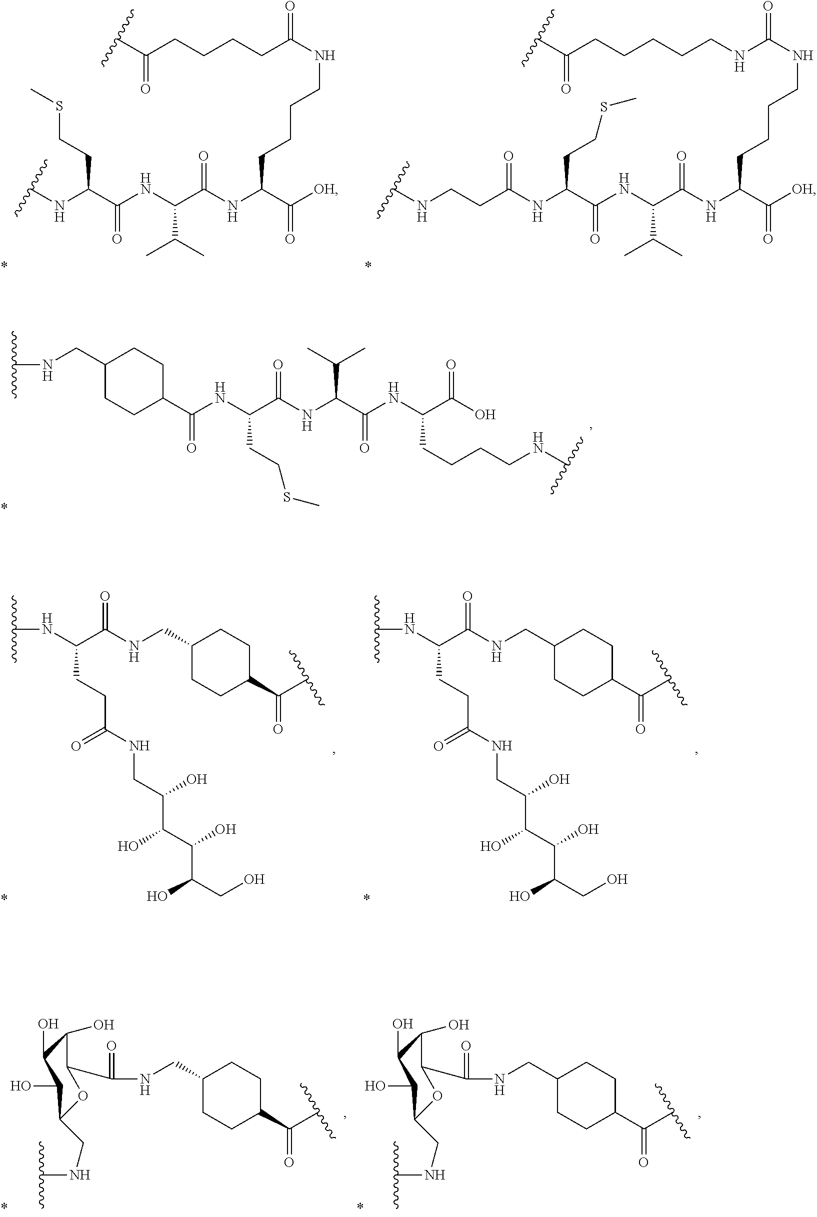

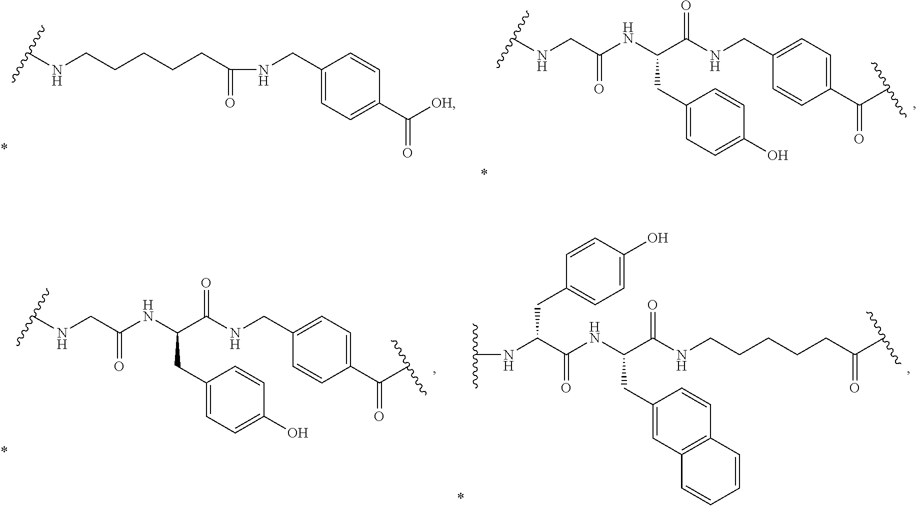 [SMILES: *NCCCCCC(=O)NCc1ccc(C(=O)O)cc1 ; *NCC(=O)N[C@@H](Cc1ccc(O)cc1)C(=O)NCc1ccc(C(*)=O)cc1 ; *NCC(=O)N[C@H](Cc1ccc(O)cc1)C(=O)NCc1ccc(C(*)=O)cc1 ; *N[C@H](Cc1ccc(O)cc1)C(=O)N[C@@H](Cc1ccc2ccccc2c1)C(=O)NCCCCCC(*)=O]

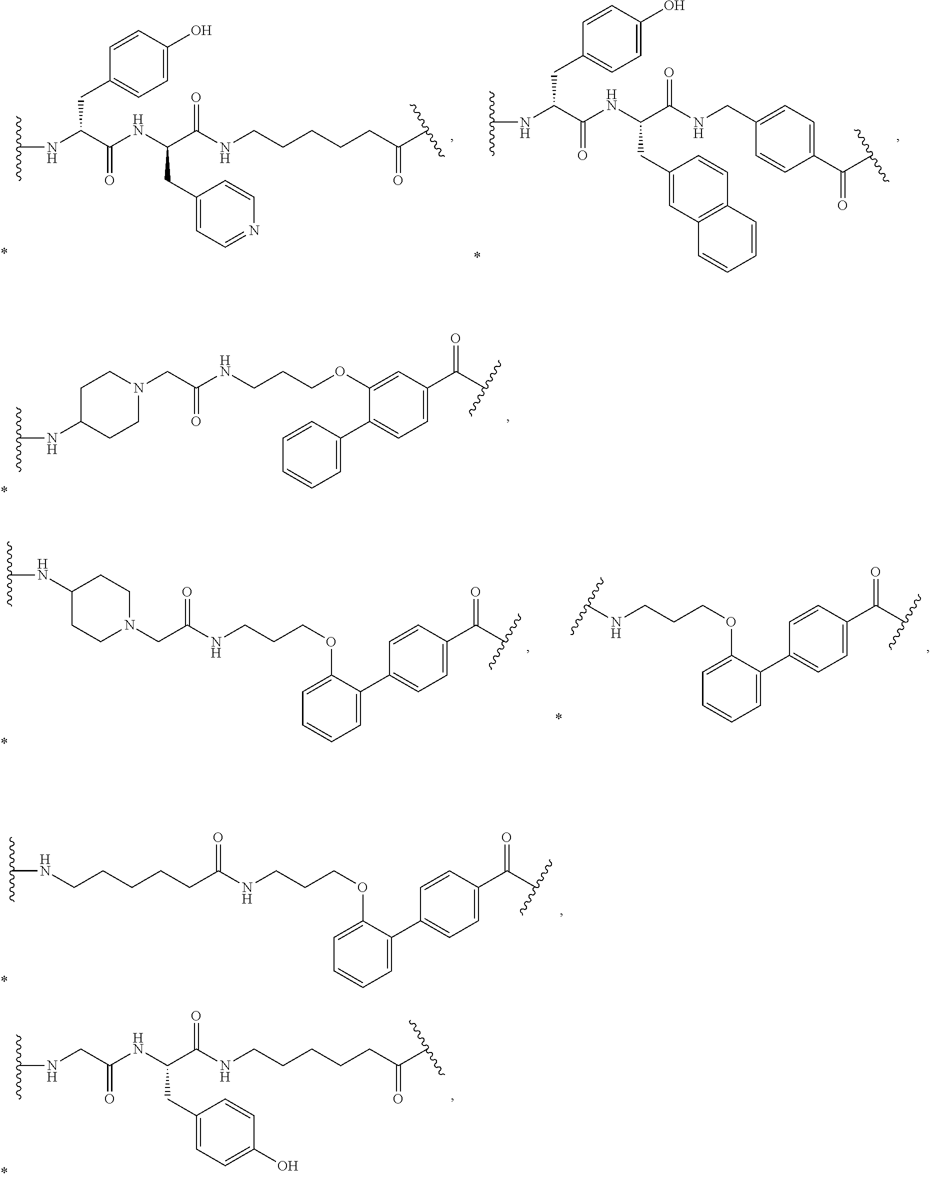

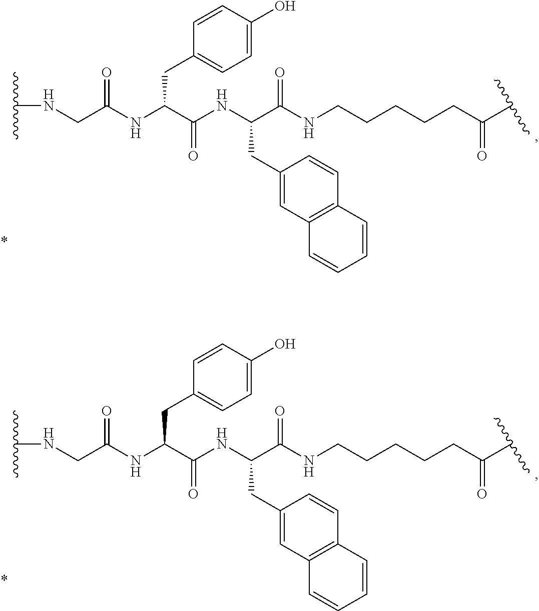 [SMILES: *NCC(=O)N[C@H](Cc1ccc(O)cc1)C(=O)N[C@@H](Cc1ccc2ccccc2c1)C(=O)NCCCCCC(*)=O ; *NCC(=O)N[C@@H](Cc1ccc(O)cc1)C(=O)N[C@@H](Cc1ccc2ccccc2c1)C(=O)NCCCCCC(*)=O]

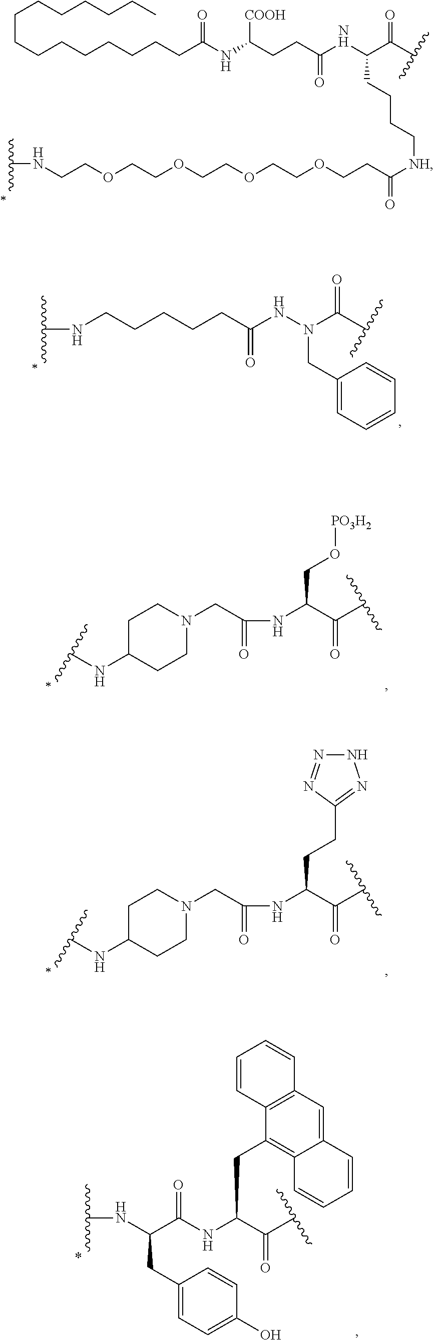

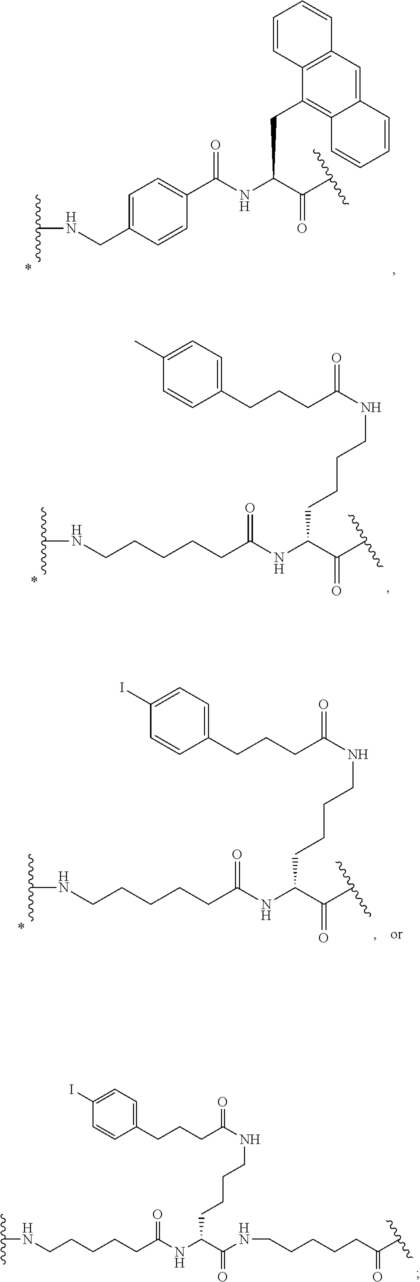 [SMILES: *NCc1ccc(C(=O)N[C@@H](Cc2c3ccccc3cc3ccccc23)C(*)=O)cc1 ; *NCCCCCC(=O)N[C@H](CCCCNC(=O)CCCc1ccc(C)cc1)C(*)=O ; *NCCCCCC(=O)N[C@H](CCCCNC(=O)CCCc1ccc(I)cc1)C(*)=O ; *NCCCCCC(=O)N[C@H](CCCCNC(=O)CCCc1ccc(I)cc1)C(=O)NCCCCCC(*)=O]

m is 1, 2, 3, 4, 5 or 6;
z is 1, 2, 3, 4, 5, or 6;
w is 1, 2, 3, 4, 5, or 6;
wherein * denotes the attachment point to R^{a}.

In some embodiments, -L- is:
absent,

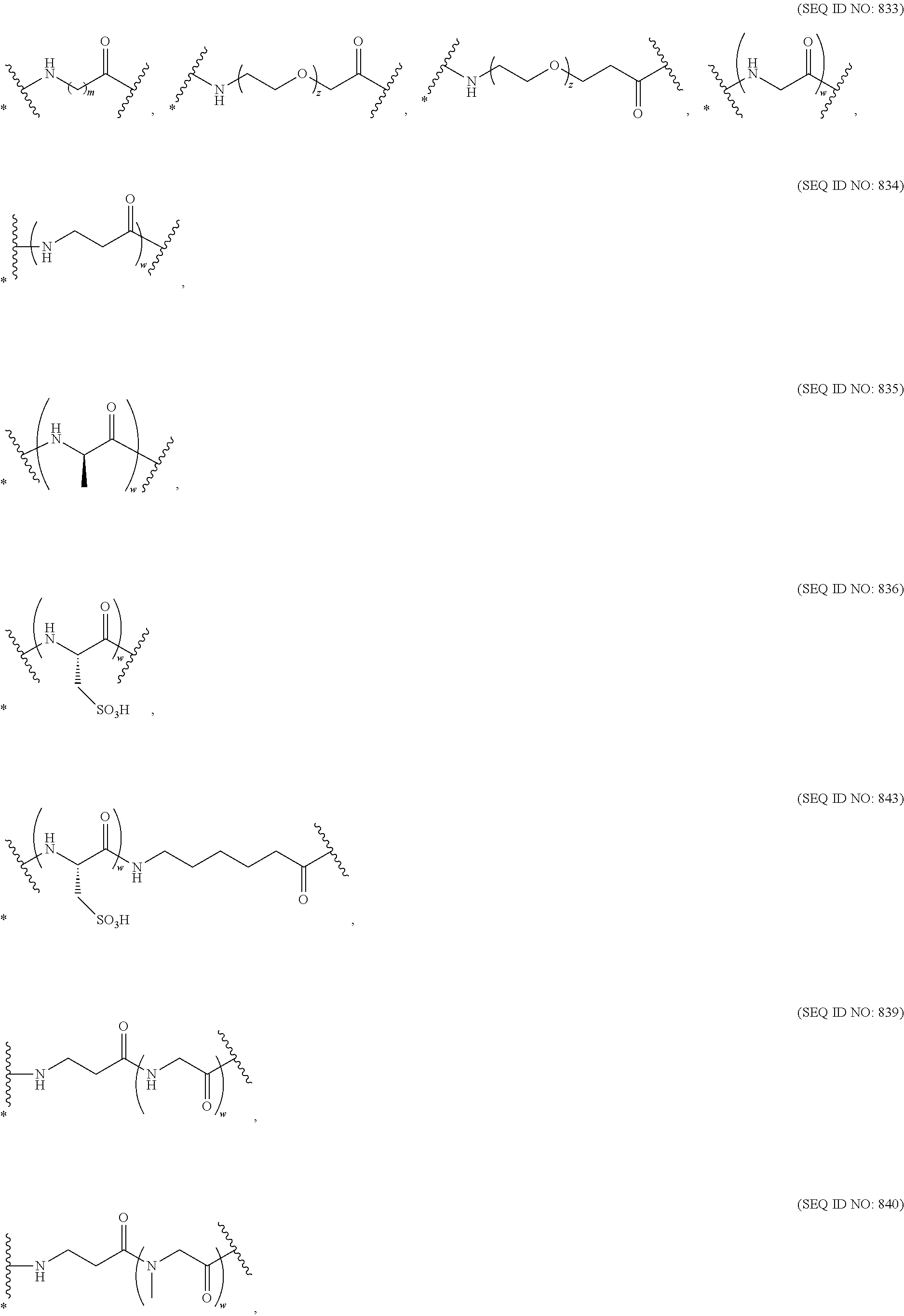

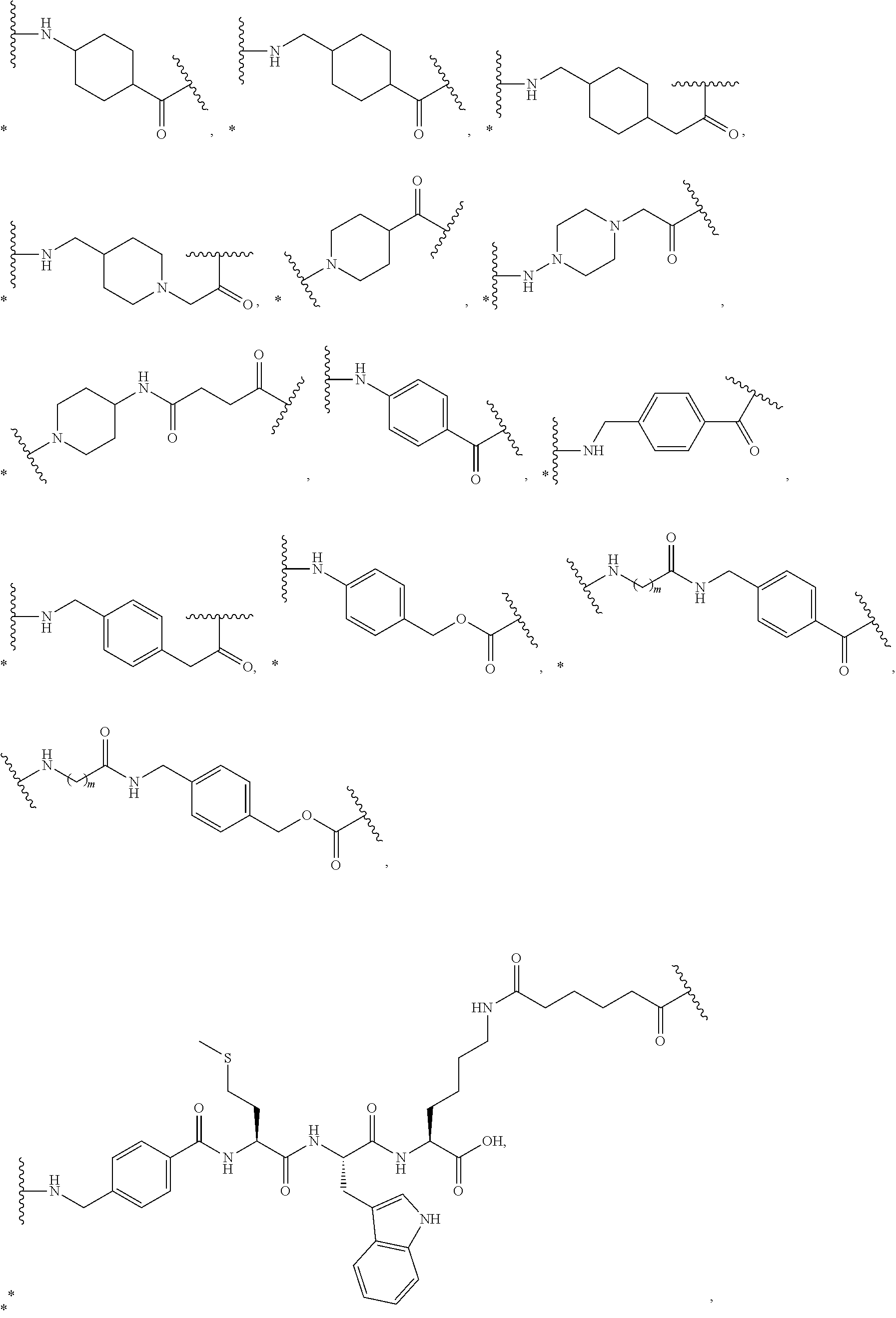

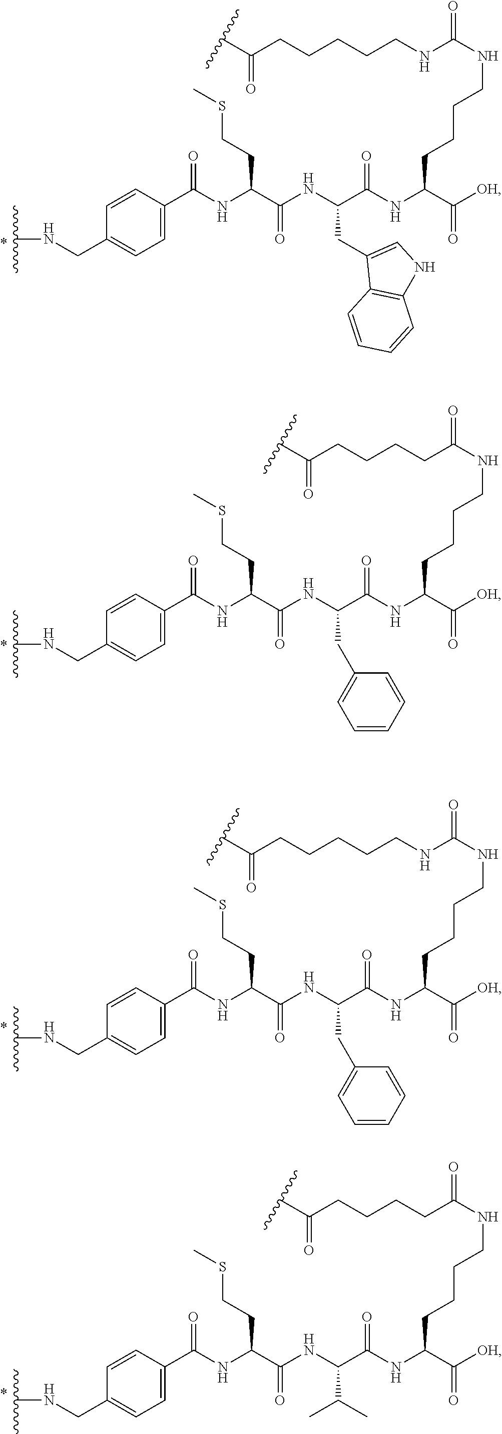 [SMILES: *NCc1ccc(C(=O)N[C@@H](CCSC)C(=O)N[C@@H](Cc2c[nH]c3ccccc23)C(=O)N[C@@H](CCCCNC(=O)NCCCCCC(*)=O)C(=O)O)cc1 ; *NCc1ccc(C(=O)N[C@@H](CCSC)C(=O)N[C@@H](Cc2ccccc2)C(=O)N[C@@H](CCCCNC(=O)CCCCC(*)=O)C(=O)O)cc1 ; *NCc1ccc(C(=O)N[C@@H](CCSC)C(=O)N[C@@H](Cc2ccccc2)C(=O)N[C@@H](CCCCNC(=O)NCCCCCC(*)=O)C(=O)O)cc1 ; *NCc1ccc(C(=O)N[C@@H](CCSC)C(=O)N[C@H](C(=O)N[C@@H](CCCCNC(=O)CCCCC(*)=O)C(=O)O)C(C)C)cc1]

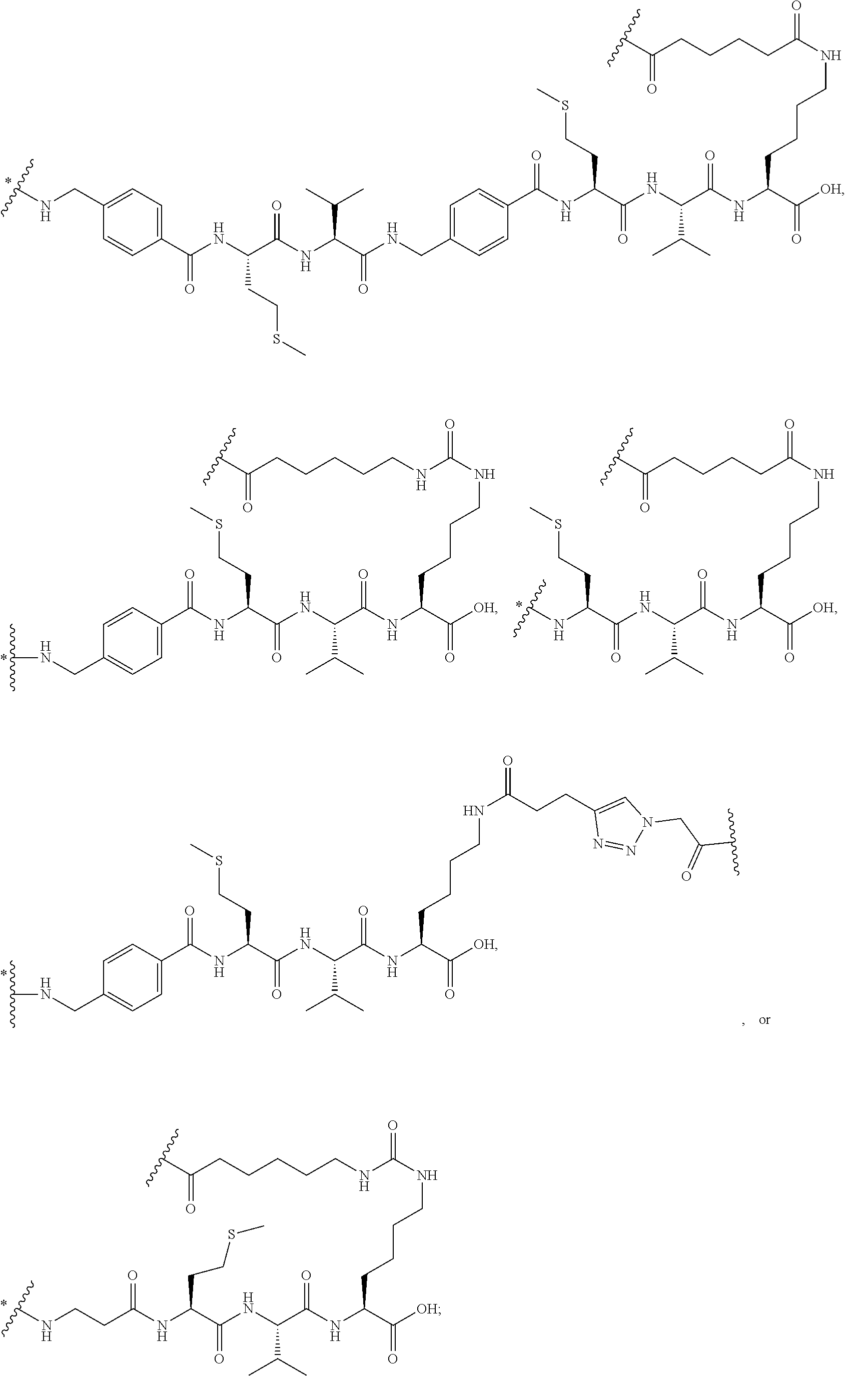 [SMILES: *NCc1ccc(C(=O)N[C@@H](CCSC)C(=O)N[C@H](C(=O)NCc2ccc(C(=O)N[C@@H](CCSC)C(=O)N[C@H](C(=O)N[C@@H](CCCCNC(=O)CCCCC(*)=O)C(=O)O)C(C)C)cc2)C(C)C)cc1 ; *NCc1ccc(C(=O)N[C@@H](CCSC)C(=O)N[C@H](C(=O)N[C@@H](CCCCNC(=O)NCCCCCC(*)=O)C(=O)O)C(C)C)cc1 ; *N[C@@H](CCSC)C(=O)N[C@H](C(=O)N[C@@H](CCCCNC(=O)CCCCC(*)=O)C(=O)O)C(C)C ; *NCc1ccc(C(=O)N[C@@H](CCSC)C(=O)N[C@H](C(=O)N[C@@H](CCCCNC(=O)CCc2cn(CC(*)=O)nn2)C(=O)O)C(C)C)cc1 ; *NCCC(=O)N[C@@H](CCSC)C(=O)N[C@H](C(=O)N[C@@H](CCCCNC(=O)NCCCCCC(*)=O)C(=O)O)C(C)C]

m is 1, 2, 3, 4, 5, or 6;
wherein * denotes the attachment point to R^{a}.

In some embodiments, m is 1. In some embodiments, m is 2. In some embodiments, m is 3. In some embodiments, m is 4. In some embodiments, m is 5. In some embodiments, m is 6.

In some embodiments, w is 1. In some embodiments, w is 2. In some embodiments, w is 3. In some embodiments, w is 4. In some embodiments, w is 5. In some embodiments, w is 6.

In some embodiments, z is 1. In some embodiments, z is 2. In some embodiments, z is 3. In some embodiments, z is 4. In some embodiments, z is 5. In some embodiments, z is 6.

In some embodiments, -L- is: absent,

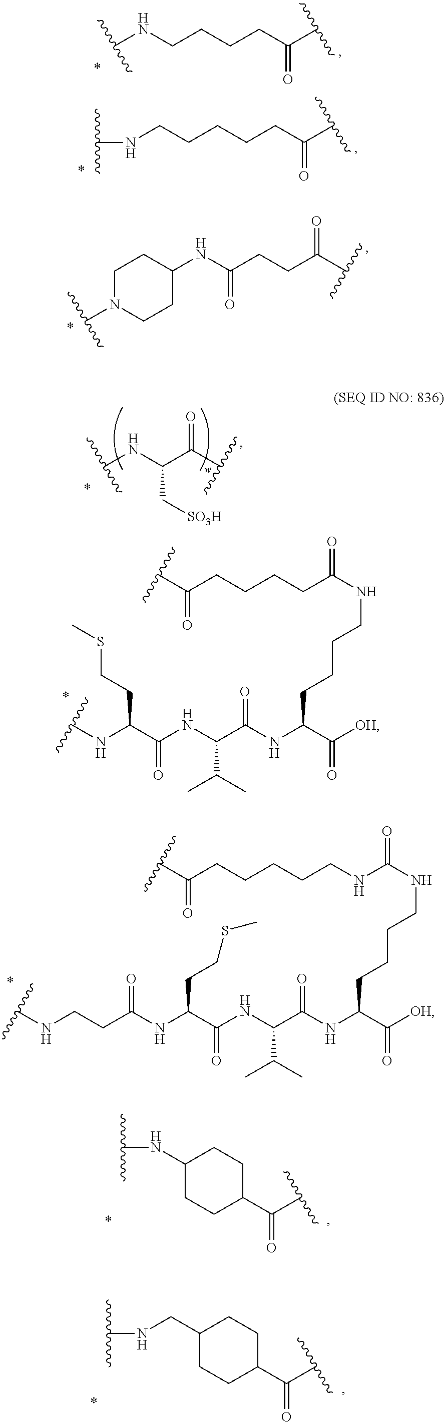

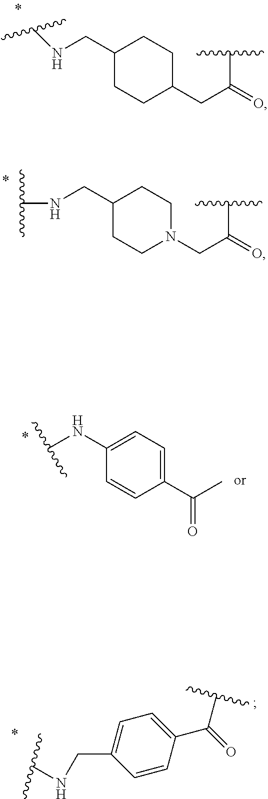 [SMILES: *NCC1CCC(CC(*)=O)CC1 ; *NCC1CCN(CC(*)=O)CC1 ; *Nc1ccc(C(C)=O)cc1 ; *NCc1ccc(C(*)=O)cc1]

wherein * denotes the attachment point to R^{a}.

### Representative Linker and Chelating Moieties

In some embodiments, R^{a}-L- is R^{a}

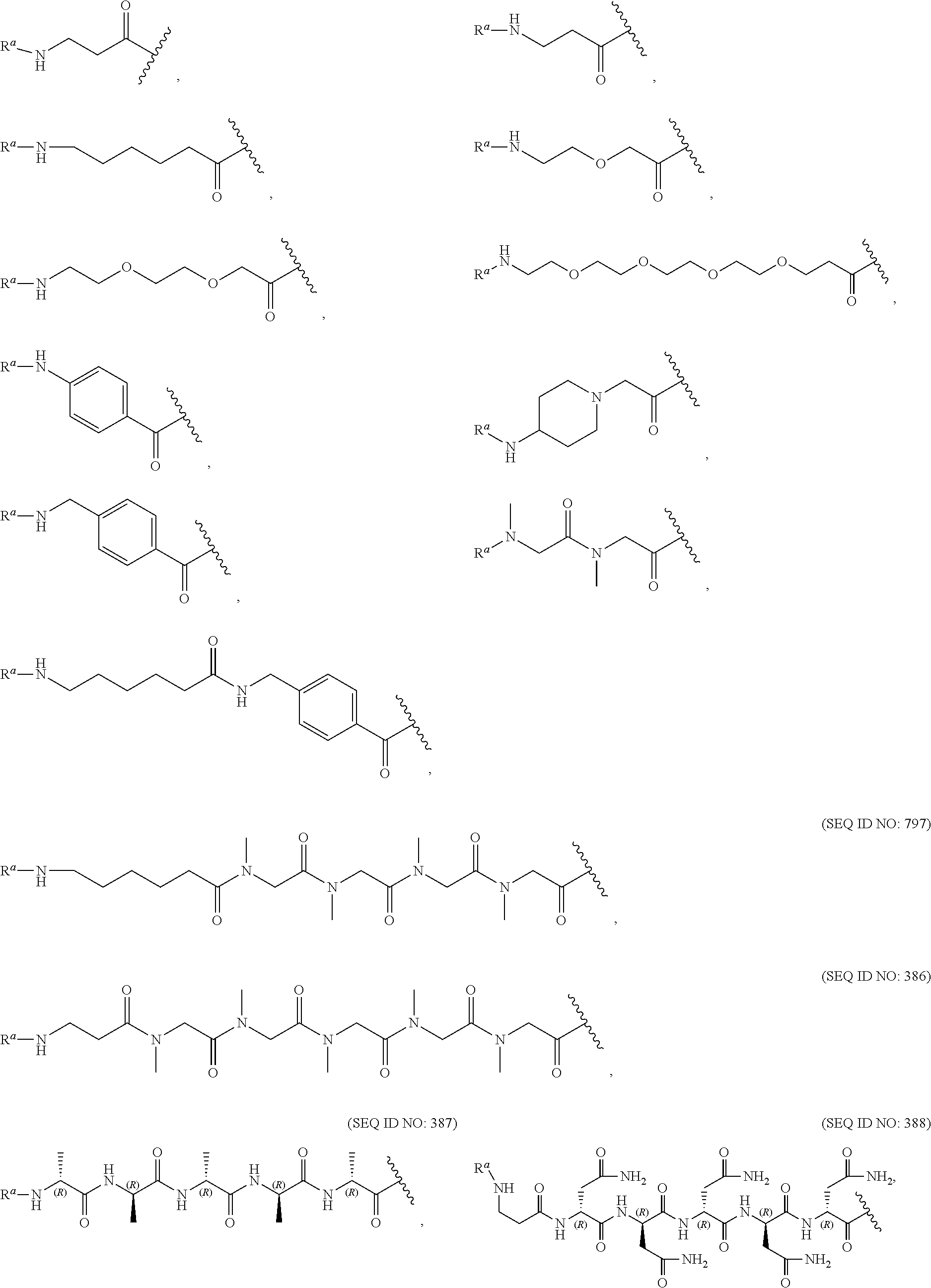

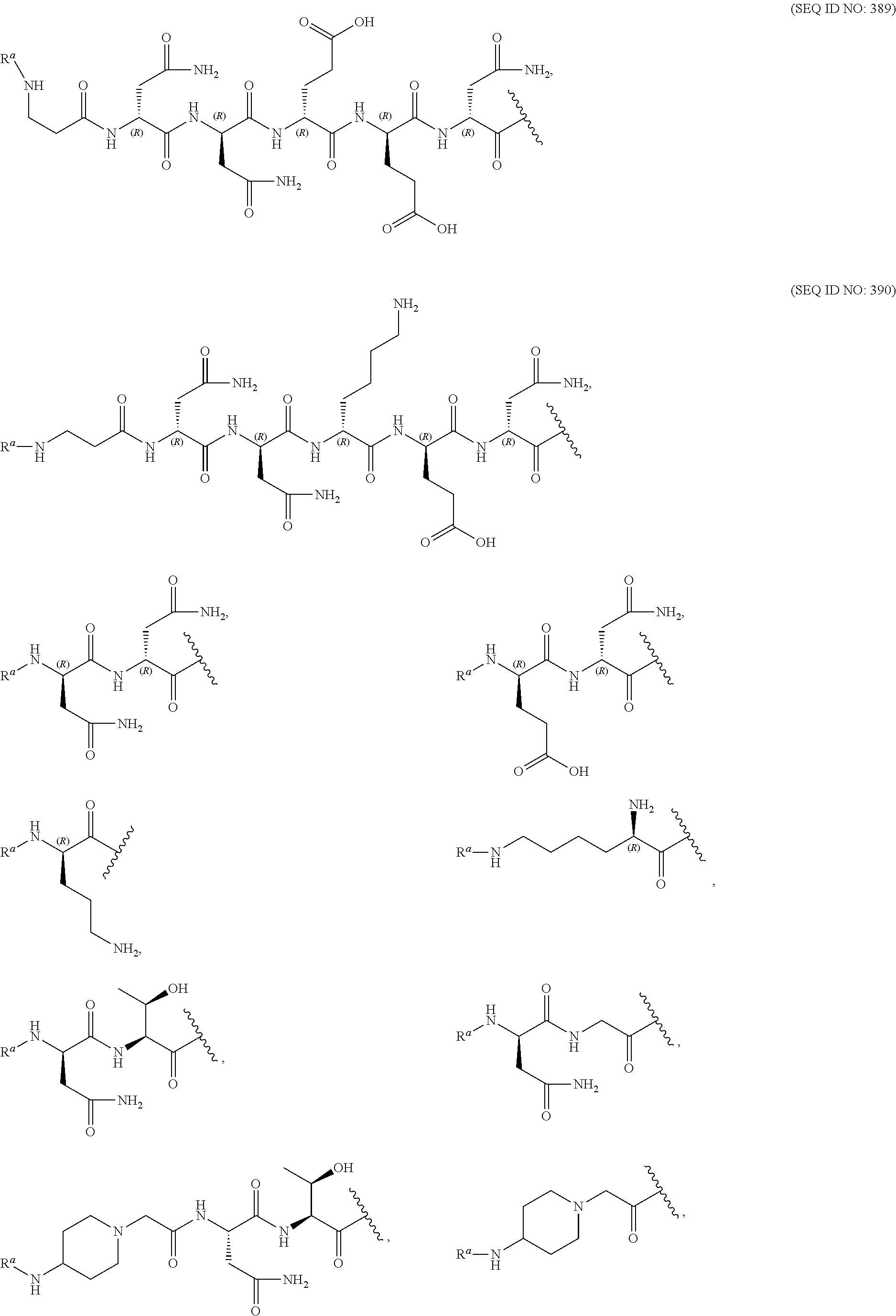

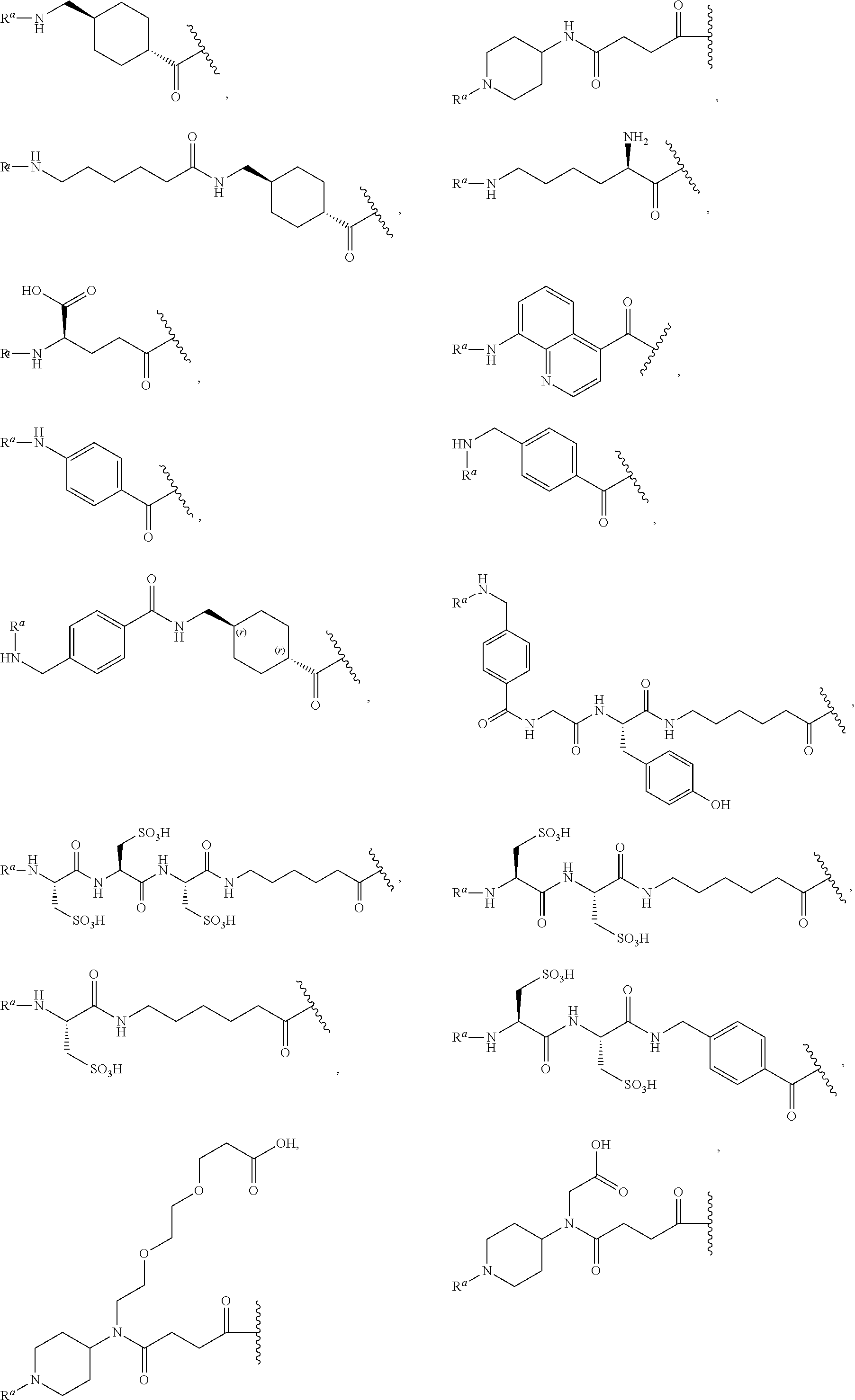

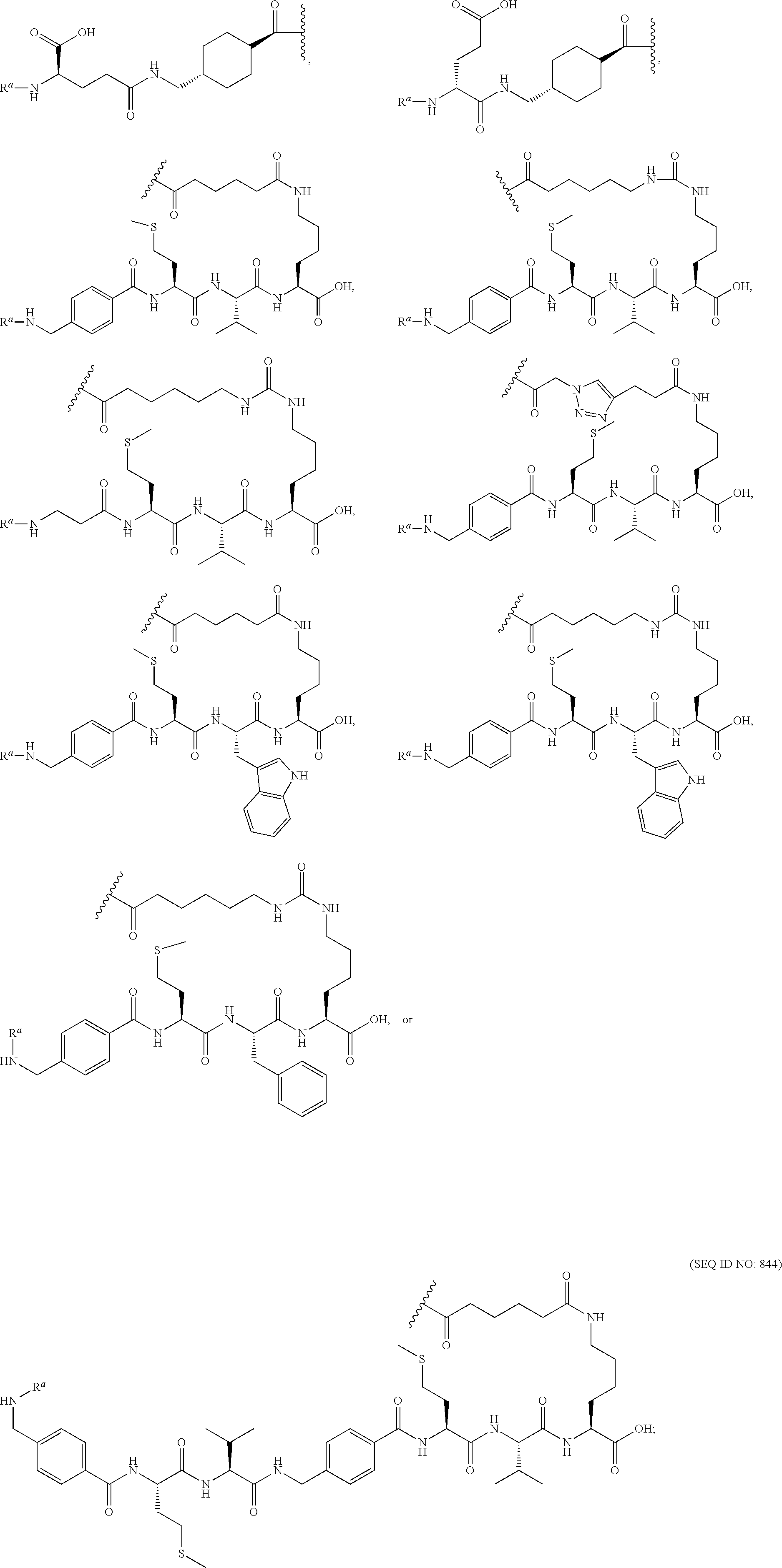

wherein R^{a} is.

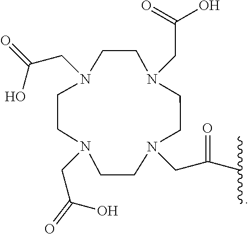 [SMILES: *C(=O)CN1CCN(CC(=O)O)CCN(CC(=O)O)CCN(CC(=O)O)CC1]

### Representative Compounds

In some embodiments, the compound of Formula (I) is compound in Table A, or a pharmaceutically acceptable salt thereof:

TABLE A

| Cmp. | |
|---|---|
| # | Structure |
| 1 | SEQ ID NO: 391 |
| 1-In | In Complex of Compound 1 |
| | SEQ ID NO: 392 |
| 1-Lu | Lu Complex of Compound 1 |
| | SEQ ID NO: 393 |
| 1-Ga | Ga Complex of Compound 1 |
| | SEQ ID NO: 394 |
| 2 | SEQ ID NO: 395 |
| 2-In | In Complex of Compound 2 |
| | SEQ ID NO: 771 |
| 3 | SEQ ID NO: 396 |
| 4 | SEQ ID NO: 397 |
| 5 | SEQ ID NO: 398 |
| 6 | SEQ ID NO: 399 |
| 6-In | In Complex of Compound 6 |
| | SEQ ID NO: 772 |
| 7 | SEQ ID NO: 400 |
| 8 | 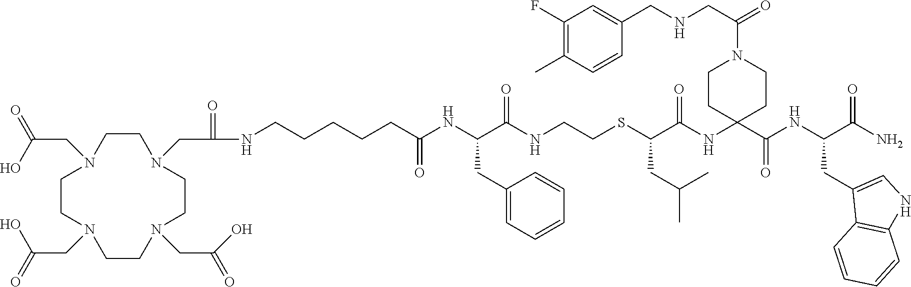 [SMILES: Cc1ccc(CNCC(=O)N2CCC(NC(=O)[C@H](CC(C)C)SCCNC(=O)[C@H](Cc3ccccc3)NC(=O)CCCCCNC(=O)CN3CCN(CC(=O)O)CCN(CC(=O)O)CCN(CC(=O)O)CC3)(C(=O)N[C@@H](Cc3c[nH]c4ccccc34)C(N)=O)CC2)cc1F] |
| 9 | 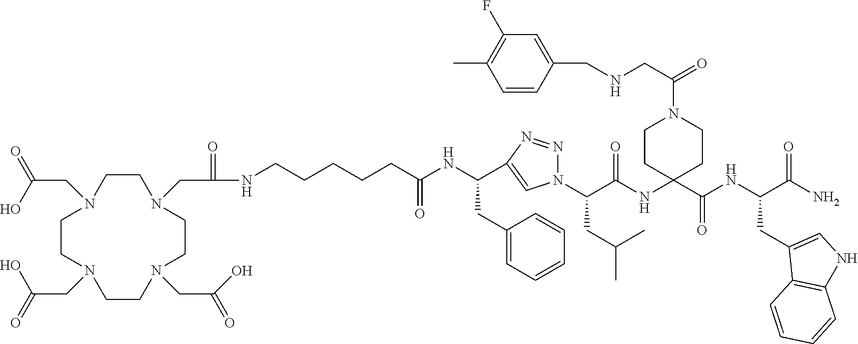 [SMILES: Cc1ccc(CNCC(=O)N2CCC(NC(=O)[C@H](CC(C)C)n3cc([C@H](Cc4ccccc4)NC(=O)CCCCCNC(=O)CN4CCN(CC(=O)O)CCN(CC(=O)O)CCN(CC(=O)O)CC4)nn3)(C(=O)N[C@@H](Cc3c[nH]c4ccccc34)C(N)=O)CC2)cc1F] |
| 10 | SEQ ID NO: 403 |
| 11 | SEQ ID NO: 404 |
| 12 | SEQ ID NO: 405 |
| 13 | SEQ ID NO: 406 |
| 14 | SEQ ID NO: 407 |
| 15 | SEQ ID NO: 408 |
| 16 | SEQ ID NO: 409 |
| 17 | SEQ ID NO: 410 |
| 18 | SEQ ID NO: 411 |
| 19 | SEQ ID NO: 412 |
| 20 | SEQ ID NO: 413 |
| 21 | SEQ ID NO: 414 |
| 22 | SEQ ID NO: 415 |
| 23 | SEQ ID NO: 416 |
| 24 | SEQ ID NO: 417 |
| 25 | SEQ ID NO: 418 |
| 26 | SEQ ID NO: 419 |
| 27 | SEQ ID NO: 420 |
| 28 | SEQ ID NO: 421 |
| 29 | SEQ ID NO: 422 |
| 30 | SEQ ID NO: 423 |
| 31 | SEQ ID NO: 424 |
| 32 | SEQ ID NO: 425 |
| 33 | SEQ ID NO: 426 |
| 34 | SEQ ID NO: 427 |
| 35 | SEQ ID NO: 428 |
| 36 | SEQ ID NO: 429 |
| 37 | SEQ ID NO: 430 |
| 38 | SEQ ID NO: 431 |
| 39 | 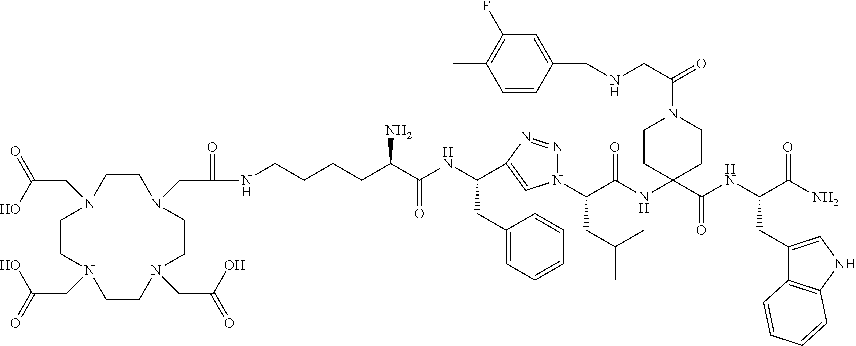 [SMILES: Cc1ccc(CNCC(=O)N2CCC(NC(=O)[C@H](CC(C)C)n3cc([C@H](Cc4ccccc4)NC(=O)[C@H](N)CCCCNC(=O)CN4CCN(CC(=O)O)CCN(CC(=O)O)CCN(CC(=O)O)CC4)nn3)(C(=O)N[C@@H](Cc3c[nH]c4ccccc34)C(N)=O)CC2)cc1F] |
| 49 | SEQ ID NO: 441 |
| 50 | SEQ ID NO: 442 |
| 51 | 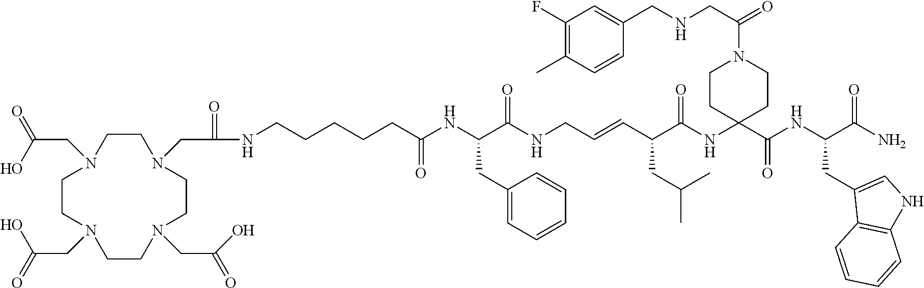 [SMILES: Cc1ccc(CNCC(=O)N2CCC(NC(=O)[C@@H](/C=C/CNC(=O)[C@H](Cc3ccccc3)NC(=O)CCCCCNC(=O)CN3CCN(CC(=O)O)CCN(CC(=O)O)CCN(CC(=O)O)CC3)CC(C)C)(C(=O)N[C@@H](Cc3c[nH]c4ccccc34)C(N)=O)CC2)cc1F] |
| 55 | SEQ ID NO: 447 |
| 57 | SEQ ID NO: 449 |
| 58 | SEQ ID NO: 450 |
| 64 | SEQ ID NO: 456 |
| 91 | SEQ ID NO: 479 |
| 104 | SEQ ID NO: 491 |
| 105 | SEQ ID NO: 492 |
| 105- | In Complex of Compound 105 |
| In | SEQ ID NO: 493 |
| 106 | SEQ ID NO: 494 |
| 107 | SEQ ID NO: 495 |
| 108 | SEQ ID NO: 496 |
| 109 | SEQ ID NO: 497 |
| 110 | SEQ ID NO: 498 |
| 113 | SEQ ID NO: 500 |
| 114 | SEQ ID NO: 501 |
| 115 | SEQ ID NO: 502 |
| 116 | SEQ ID NO: 503 |
| 117 | SEQ ID NO: 504 |
| 118 | SEQ ID NO: 505 |
| 118-Lu | Lu Complex of Compound 118 |
| | 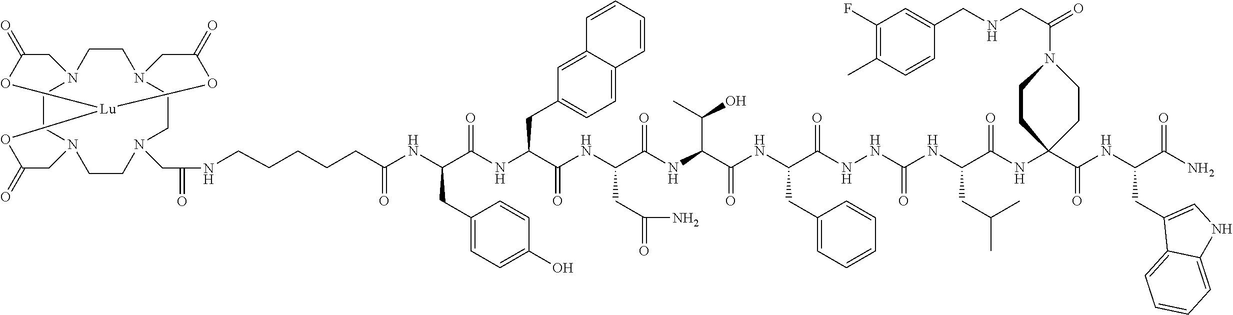 |
| 119 | SEQ ID NO: 506 |
| 120 | SEQ ID NO: 507 |
| 121 | SEQ ID NO: 508 |
| 123 | SEQ ID NO: 510 |
| 130 | SEQ ID NO: 511 |
| 131 | SEQ ID NO: 512 |
| 132 | SEQ ID NO: 513 |
| 133 | SEQ ID NO: 514 |
| 134 | 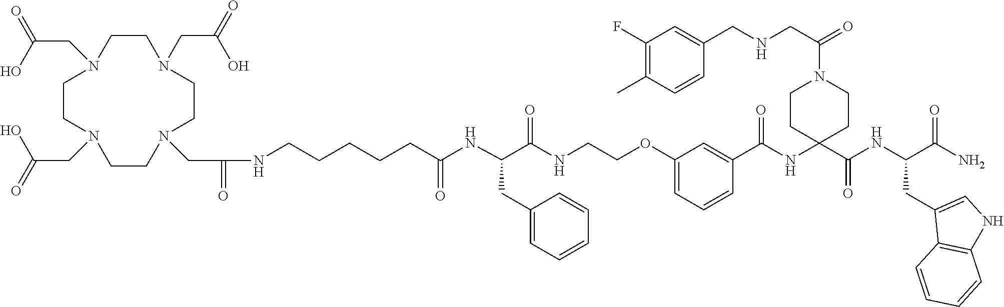 [SMILES: Cc1ccc(CNCC(=O)N2CCC(NC(=O)c3cccc(OCCNC(=O)[C@H](Cc4ccccc4)NC(=O)CCCCCNC(=O)CN4CCN(CC(=O)O)CCN(CC(=O)O)CCN(CC(=O)O)CC4)c3)(C(=O)N[C@@H](Cc3c[nH]c4ccccc34)C(N)=O)CC2)cc1F] |
| 135 | 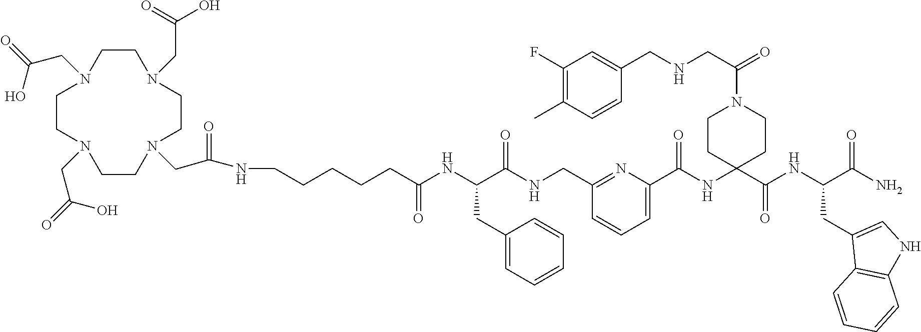 [SMILES: Cc1ccc(CNCC(=O)N2CCC(NC(=O)c3cccc(CNC(=O)[C@H](Cc4ccccc4)NC(=O)CCCCCNC(=O)CN4CCN(CC(=O)O)CCN(CC(=O)O)CCN(CC(=O)O)CC4)n3)(C(=O)N[C@@H](Cc3c[nH]c4ccccc34)C(N)=O)CC2)cc1F] |
| 136 | 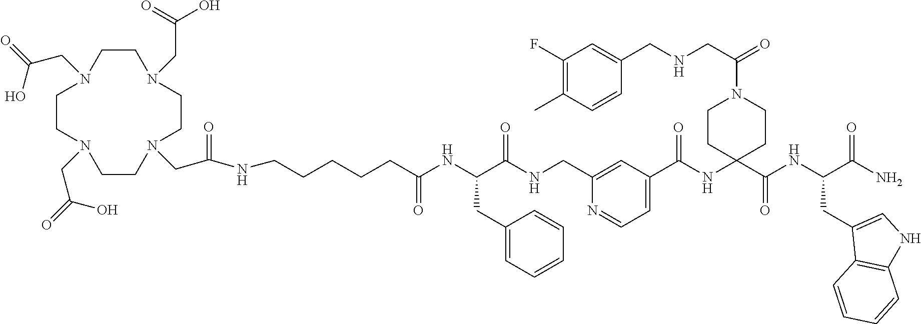 [SMILES: Cc1ccc(CNCC(=O)N2CCC(NC(=O)c3ccnc(CNC(=O)[C@H](Cc4ccccc4)NC(=O)CCCCCNC(=O)CN4CCN(CC(=O)O)CCN(CC(=O)O)CCN(CC(=O)O)CC4)c3)(C(=O)N[C@@H](Cc3c[nH]c4ccccc34)C(N)=O)CC2)cc1F] |
| 137 | 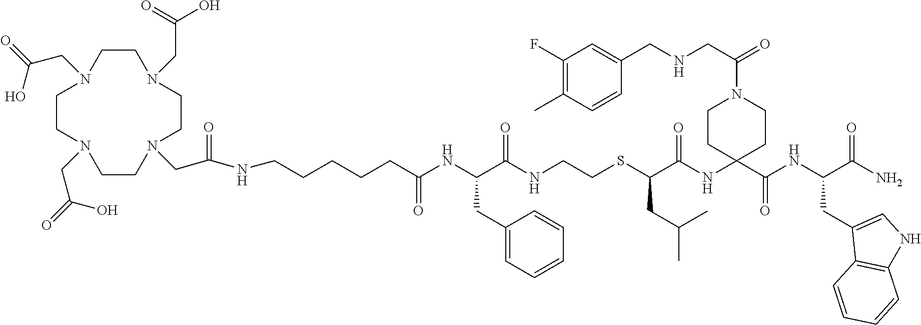 [SMILES: Cc1ccc(CNCC(=O)N2CCC(NC(=O)[C@@H](CC(C)C)SCCNC(=O)[C@H](Cc3ccccc3)NC(=O)CCCCCNC(=O)CN3CCN(CC(=O)O)CCN(CC(=O)O)CCN(CC(=O)O)CC3)(C(=O)N[C@@H](Cc3c[nH]c4ccccc34)C(N)=O)CC2)cc1F] |
| 138 | 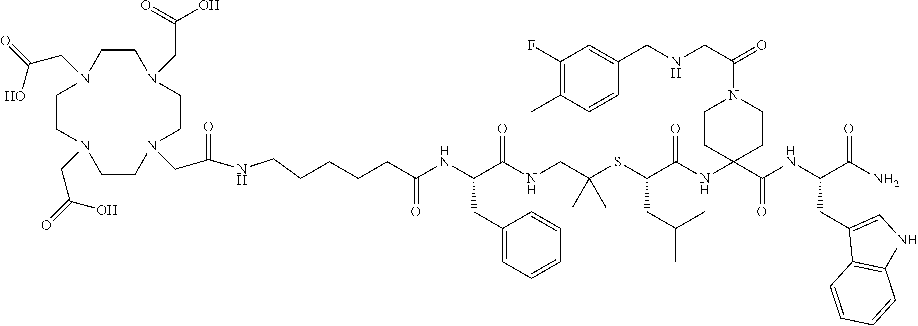 [SMILES: Cc1ccc(CNCC(=O)N2CCC(NC(=O)[C@H](CC(C)C)SC(C)(C)CNC(=O)[C@H](Cc3ccccc3)NC(=O)CCCCCNC(=O)CN3CCN(CC(=O)O)CCN(CC(=O)O)CCN(CC(=O)O)CC3)(C(=O)N[C@@H](Cc3c[nH]c4ccccc34)C(N)=O)CC2)cc1F] |
| 139 | 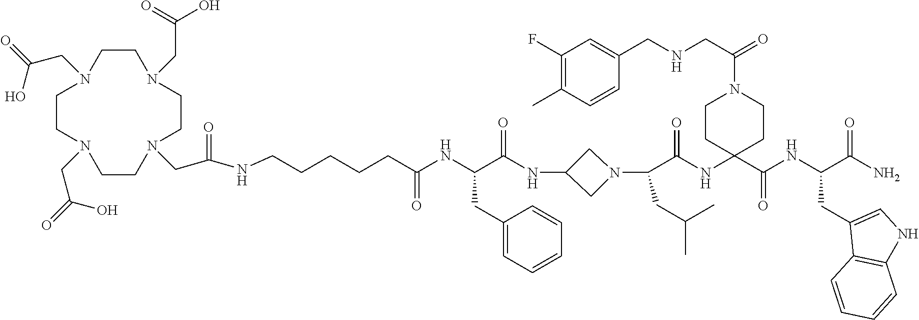 [SMILES: Cc1ccc(CNCC(=O)N2CCC(NC(=O)[C@H](CC(C)C)N3CC(NC(=O)[C@H](Cc4ccccc4)NC(=O)CCCCCNC(=O)CN4CCN(CC(=O)O)CCN(CC(=O)O)CCN(CC(=O)O)CC4)C3)(C(=O)N[C@@H](Cc3c[nH]c4ccccc34)C(N)=O)CC2)cc1F] |
| 140 | SEQ ID NO: 521 |
| 141 | 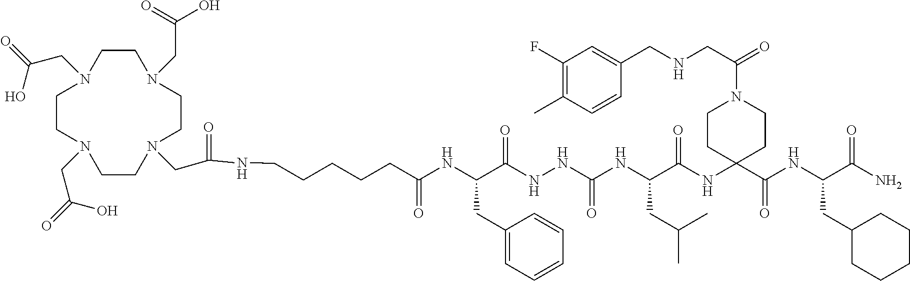 [SMILES: Cc1ccc(CNCC(=O)N2CCC(NC(=O)[C@H](CC(C)C)NC(=O)NNC(=O)[C@H](Cc3ccccc3)NC(=O)CCCCCNC(=O)CN3CCN(CC(=O)O)CCN(CC(=O)O)CCN(CC(=O)O)CC3)(C(=O)N[C@@H](CC3CCCCC3)C(N)=O)CC2)cc1F] |
| 142 | 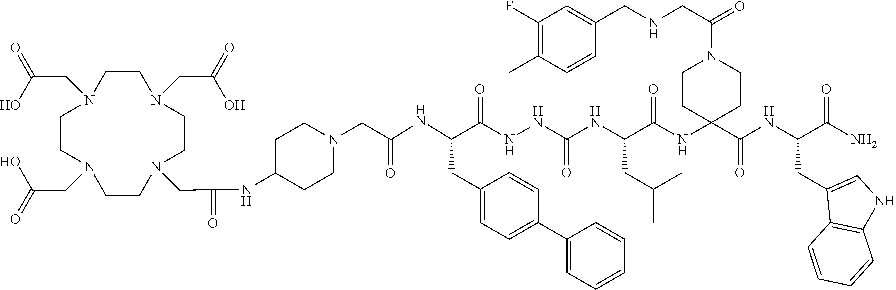 [SMILES: Cc1ccc(CNCC(=O)N2CCC(NC(=O)[C@H](CC(C)C)NC(=O)NNC(=O)[C@H](Cc3ccc(-c4ccccc4)cc3)NC(=O)CN3CCC(NC(=O)CN4CCN(CC(=O)O)CCN(CC(=O)O)CCN(CC(=O)O)CC4)CC3)(C(=O)N[C@@H](Cc3c[nH]c4ccccc34)C(N)=O)CC2)cc1F] |
| 146 | SEQ ID NO: 527 |
| 147 | 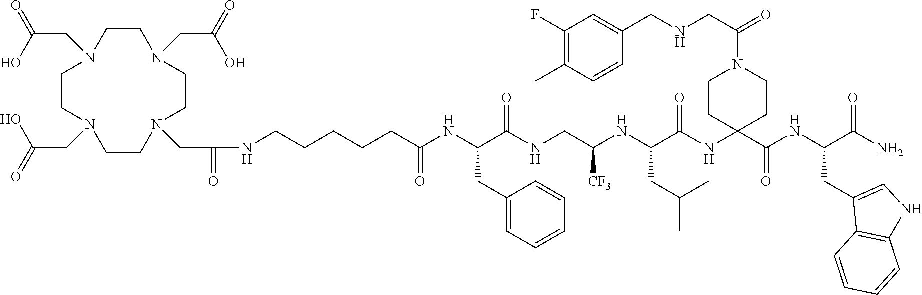 [SMILES: Cc1ccc(CNCC(=O)N2CCC(NC(=O)[C@H](CC(C)C)N[C@H](CNC(=O)[C@H](Cc3ccccc3)NC(=O)CCCCCNC(=O)CN3CCN(CC(=O)O)CCN(CC(=O)O)CCN(CC(=O)O)CC3)C(F)(F)F)(C(=O)N[C@@H](Cc3c[nH]c4ccccc34)C(N)=O)CC2)cc1F] |
| 148 | 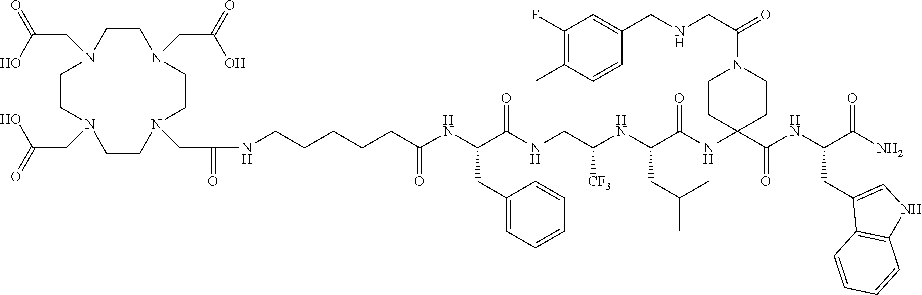 [SMILES: Cc1ccc(CNCC(=O)N2CCC(NC(=O)[C@H](CC(C)C)N[C@@H](CNC(=O)[C@H](Cc3ccccc3)NC(=O)CCCCCNC(=O)CN3CCN(CC(=O)O)CCN(CC(=O)O)CCN(CC(=O)O)CC3)C(F)(F)F)(C(=O)N[C@@H](Cc3c[nH]c4ccccc34)C(N)=O)CC2)cc1F] |
| 149 | SEQ ID NO: 530 |
| 151 | SEQ ID NO: 532 |
| 156 | SEQ ID NO: 537 |
| 158 | SEQ ID NO: 539 |
| 159 | SEQ ID NO: 540 |
| 160 | SEQ ID NO: 541 |
| 161 | SEQ ID NO: 542 |
| 164 | SEQ ID NO: 545 |
| 165 | SEQ ID NO: 546 |
| 166 | SEQ ID NO: 547 |
| 167 | SEQ ID NO: 548 |
| 168 | SEQ ID NO: 549 |
| 169 | SEQ ID NO: 550 |
| 170 | SEQ ID NO: 551 |
| 171 | SEQ ID NO: 552 |
| 176 | SEQ ID NO: 555 |
| 181 | SEQ ID NO: 560 |
| 182 | SEQ ID NO: 561 |
| 183 | SEQ ID NO: 562 |
| 184 | SEQ ID NO: 563 |
| 185 | SEQ ID NO: 564 |
| 186 | SEQ ID NO: 565 |
| 187 | SEQ ID NO: 566 |
| 188 | SEQ ID NO: 567 |
| 189 | SEQ ID NO: 568 |
| 192 | SEQ ID NO: 571 |
| 193 | SEQ ID NO: 572 |
| 193- | Indium complex of Compound 193 |
| In | SEQ ID NO: 573 |
| 195 | SEQ ID NO: 575 |
| 196 | SEQ ID NO: 576 |
| 196- | In Complex of Compound 196 |
| In | SEQ ID NO: 577 |
| 197 | SEQ ID NO: 578 |
| 198 | SEQ ID NO: 579 |
| 199 | SEQ ID NO: 580 |
| 201 | SEQ ID NO: 581 |
| 201- | In Complex of Compound 201 |
| In | SEQ ID NO: 582 |
| 202 | SEQ ID NO: 583 |
| 203 | SEQ ID NO: 584 |
| 204 | 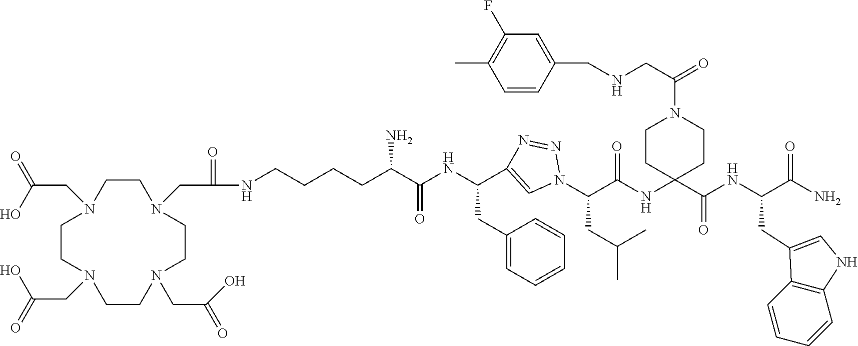 [SMILES: Cc1ccc(CNCC(=O)N2CCC(NC(=O)[C@H](CC(C)C)n3cc([C@H](Cc4ccccc4)NC(=O)[C@@H](N)CCCCNC(=O)CN4CCN(CC(=O)O)CCN(CC(=O)O)CCN(CC(=O)O)CC4)nn3)(C(=O)N[C@@H](Cc3c[nH]c4ccccc34)C(N)=O)CC2)cc1F] |
| 205 | SEQ ID NO: 586 |
| 206 | SEQ ID NO: 587 |
| 213 | SEQ ID NO: 594 |
| 214 | 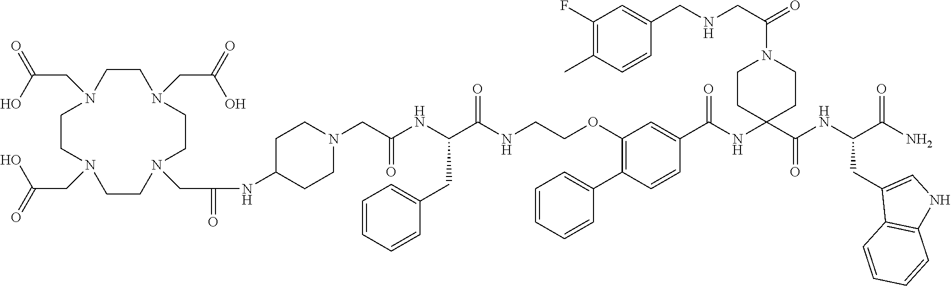 [SMILES: Cc1ccc(CNCC(=O)N2CCC(NC(=O)c3ccc(-c4ccccc4)c(OCCNC(=O)[C@H](Cc4ccccc4)NC(=O)CN4CCC(NC(=O)CN5CCN(CC(=O)O)CCN(CC(=O)O)CCN(CC(=O)O)CC5)CC4)c3)(C(=O)N[C@@H](Cc3c[nH]c4ccccc34)C(N)=O)CC2)cc1F] |
| 215 | 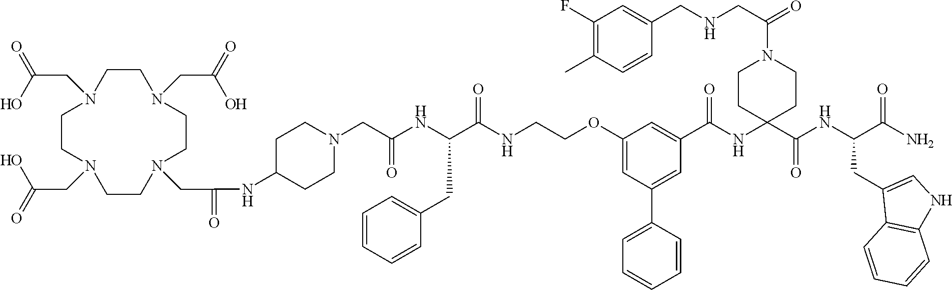 [SMILES: Cc1ccc(CNCC(=O)N2CCC(NC(=O)c3cc(OCCNC(=O)[C@H](Cc4ccccc4)NC(=O)CN4CCC(NC(=O)CN5CCN(CC(=O)O)CCN(CC(=O)O)CCN(CC(=O)O)CC5)CC4)cc(-c4ccccc4)c3)(C(=O)N[C@@H](Cc3c[nH]c4ccccc34)C(N)=O)CC2)cc1F] |
| 216 | 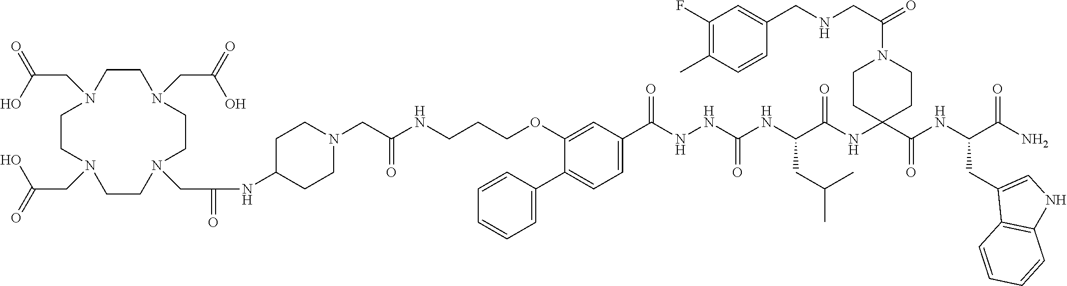 [SMILES: Cc1ccc(CNCC(=O)N2CCC(NC(=O)[C@H](CC(C)C)NC(=O)NNC(=O)c3ccc(-c4ccccc4)c(OCCCNC(=O)CN4CCC(NC(=O)CN5CCN(CC(=O)O)CCN(CC(=O)O)CCN(CC(=O)O)CC5)CC4)c3)(C(=O)N[C@@H](Cc3c[nH]c4ccccc34)C(N)=O)CC2)cc1F] |
| 217 | 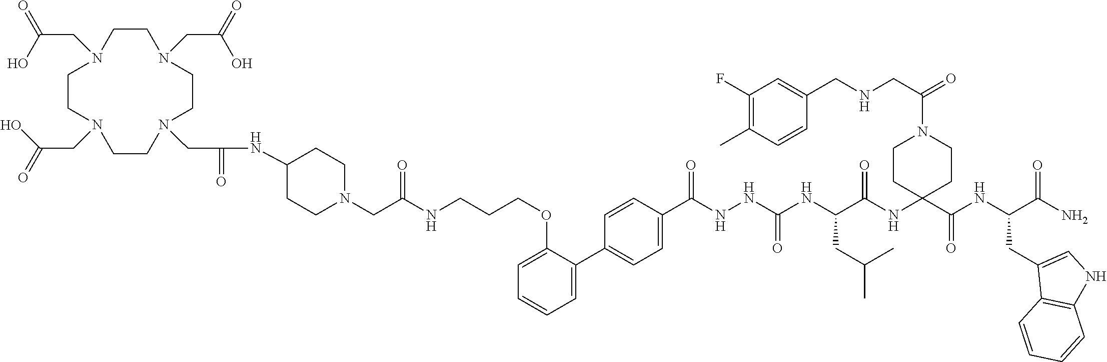 [SMILES: Cc1ccc(CNCC(=O)N2CCC(NC(=O)[C@H](CC(C)C)NC(=O)NNC(=O)c3ccc(-c4ccccc4OCCCNC(=O)CN4CCC(NC(=O)CN5CCN(CC(=O)O)CCN(CC(=O)O)CCN(CC(=O)O)CC5)CC4)cc3)(C(=O)N[C@@H](Cc3c[nH]c4ccccc34)C(N)=O)CC2)cc1F] |
| 218 | SEQ ID NO: 599 |
| 221 | SEQ ID NO: 602 |
| 222 | SEQ ID NO: 603 |
| 226 | SEQ ID NO: 607 |
| 230 | SEQ ID NO: 611 |
| 231 | SEQ ID NO: 612 |
| 232 | SEQ ID NO: 613 |
| 233 | SEQ ID NO: 614 |
| 234 | SEQ ID NO: 615 |
| 235 | SEQ ID NO: 616 |
| 236 | SEQ ID NO: 617 |
| 237 | SEQ ID NO: 618 |
| 238 | SEQ ID NO: 619 |
| 239 | SEQ ID NO: 620 |
| 240 | SEQ ID NO: 621 |
| 241 | SEQ ID NO: 622 |
| 242 | SEQ ID NO: 623 |
| 244 | SEQ ID NO: 625 |
| 245 | SEQ ID NO: 626 |
| 246 | 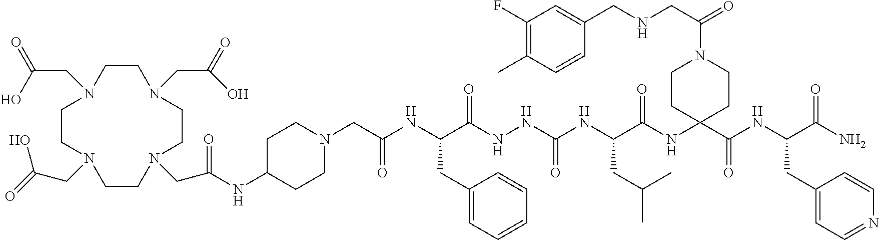 [SMILES: Cc1ccc(CNCC(=O)N2CCC(NC(=O)[C@H](CC(C)C)NC(=O)NNC(=O)[C@H](Cc3ccccc3)NC(=O)CN3CCC(NC(=O)CN4CCN(CC(=O)O)CCN(CC(=O)O)CCN(CC(=O)O)CC4)CC3)(C(=O)N[C@@H](Cc3ccncc3)C(N)=O)CC2)cc1F] |
| 247 | SEQ ID NO: 628 |
| 248 | SEQ ID NO: 629 |
| 249 | SEQ ID NO: 630 |
| 250 | SEQ ID NO: 631 |
| 251 | SEQ ID NO: 632 |
| 252 | SEQ ID NO: 633 |
| 253 | SEQ ID NO: 634 |
| 254 | SEQ ID NO: 635 |
| 255 | SEQ ID NO: 636 |
| 256 | SEQ ID NO: 637 |
| 258 | SEQ ID NO: 639 |
| 259 | SEQ ID NO: 640 |
| 260 | SEQ ID NO: 641 |
| 261 | 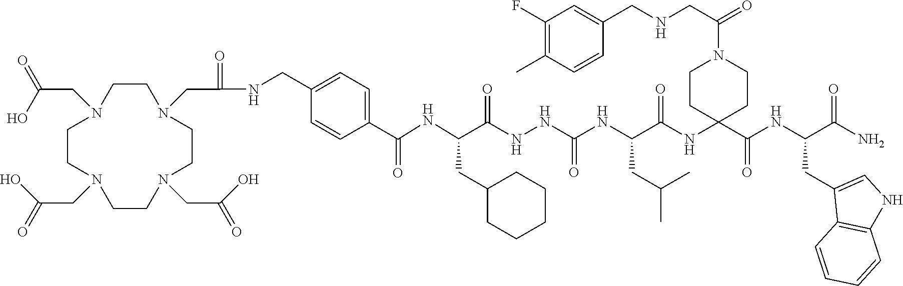 [SMILES: Cc1ccc(CNCC(=O)N2CCC(NC(=O)[C@H](CC(C)C)NC(=O)NNC(=O)[C@H](CC3CCCCC3)NC(=O)c3ccc(CNC(=O)CN4CCN(CC(=O)O)CCN(CC(=O)O)CCN(CC(=O)O)CC4)cc3)(C(=O)N[C@@H](Cc3c[nH]c4ccccc34)C(N)=O)CC2)cc1F] |
| 264 | SEQ ID NO: 645 |
| 266 | SEQ ID NO: 647 |
| 269 | SEQ ID NO: 650 |
| 269- | In Complex of Compound 269 |
| In | SEQ ID NO: 651 |
| 270 | SEQ ID NO: 652 |
| 271 | SEQ ID NO: 653 |
| 272 | SEQ ID NO: 654 |
| 273 | SEQ ID NO: 655 |
| 275 | SEQ ID NO: 657 |
| 276 | SEQ ID NO: 658 |
| 277 | SEQ ID NO: 659 |
| 278 | SEQ ID NO: 660 |
| 279 | SEQ ID NO: 661 |
| 280 | 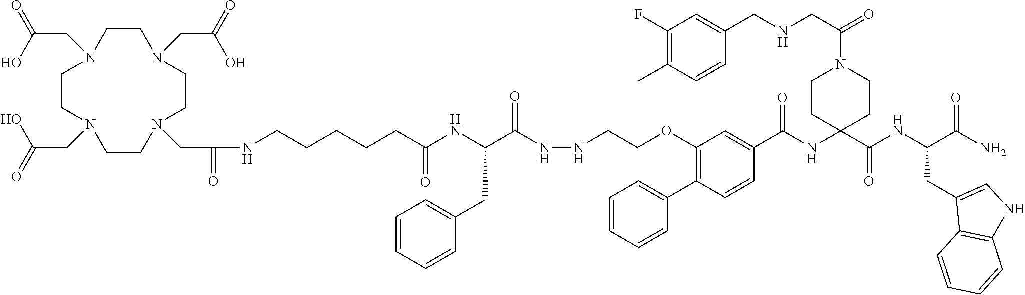 [SMILES: Cc1ccc(CNCC(=O)N2CCC(NC(=O)c3ccc(-c4ccccc4)c(OCCNNC(=O)[C@H](Cc4ccccc4)NC(=O)CCCCCNC(=O)CN4CCN(CC(=O)O)CCN(CC(=O)O)CCN(CC(=O)O)CC4)c3)(C(=O)N[C@@H](Cc3c[nH]c4ccccc34)C(N)=O)CC2)cc1F] |
| 281 | SEQ ID NO: 663 |
| 282 | 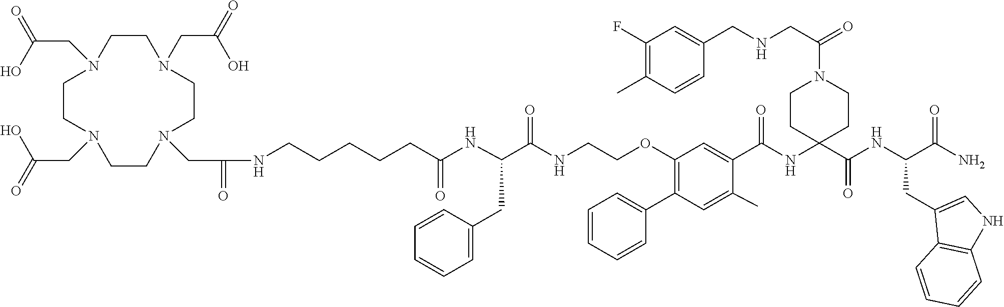 [SMILES: Cc1ccc(CNCC(=O)N2CCC(NC(=O)c3cc(OCCNC(=O)[C@H](Cc4ccccc4)NC(=O)CCCCCNC(=O)CN4CCN(CC(=O)O)CCN(CC(=O)O)CCN(CC(=O)O)CC4)c(-c4ccccc4)cc3C)(C(=O)N[C@@H](Cc3c[nH]c4ccccc34)C(N)=O)CC2)cc1F] |
| 283 | 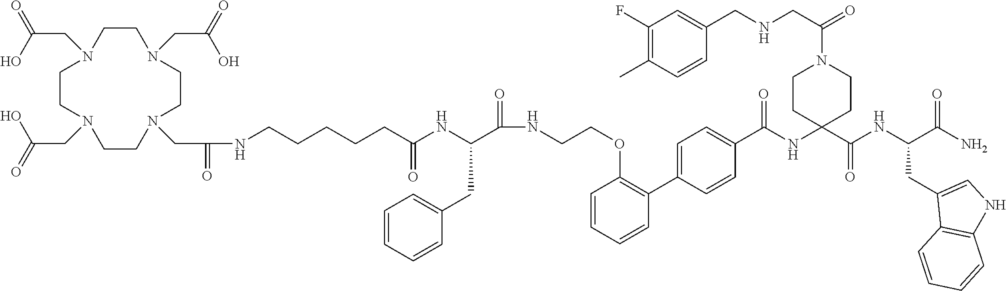 [SMILES: Cc1ccc(CNCC(=O)N2CCC(NC(=O)c3ccc(-c4ccccc4OCCNC(=O)[C@H](Cc4ccccc4)NC(=O)CCCCCNC(=O)CN4CCN(CC(=O)O)CCN(CC(=O)O)CCN(CC(=O)O)CC4)cc3)(C(=O)N[C@@H](Cc3c[nH]c4ccccc34)C(N)=O)CC2)cc1F] |
| 284 | 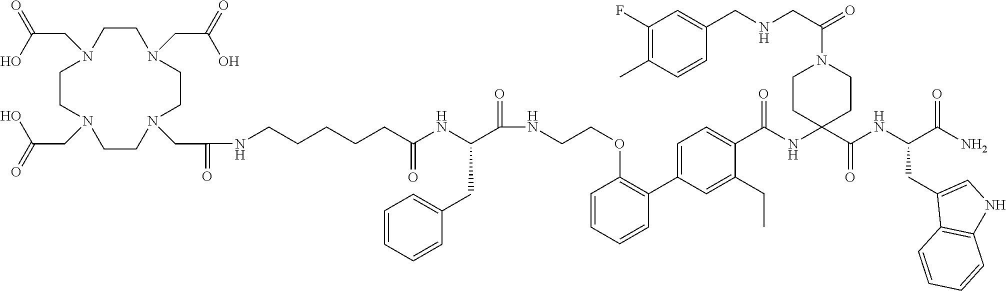 [SMILES: CCc1cc(-c2ccccc2OCCNC(=O)[C@H](Cc2ccccc2)NC(=O)CCCCCNC(=O)CN2CCN(CC(=O)O)CCN(CC(=O)O)CCN(CC(=O)O)CC2)ccc1C(=O)NC1(C(=O)N[C@@H](Cc2c[nH]c3ccccc23)C(N)=O)CCN(C(=O)CNCc2ccc(C)c(F)c2)CC1] |
| 285 | SEQ ID NO: 667 |
| 286 | SEQ ID NO: 668 |
| 287 | SEQ ID NO: 669 |
| 288 | SEQ ID NO: 670 |
| 289 | SEQ ID NO: 671 |
| 290 | SEQ ID NO: 672 |
| 291 | SEQ ID NO: 673 |
| 292 | SEQ ID NO: 674 |
| 293 | SEQ ID NO: 675 |
| 294 | SEQ ID NO: 676 |
| 295 | SEQ ID NO: 677 |
| 296 | SEQ ID NO: 678 |
| 297 | 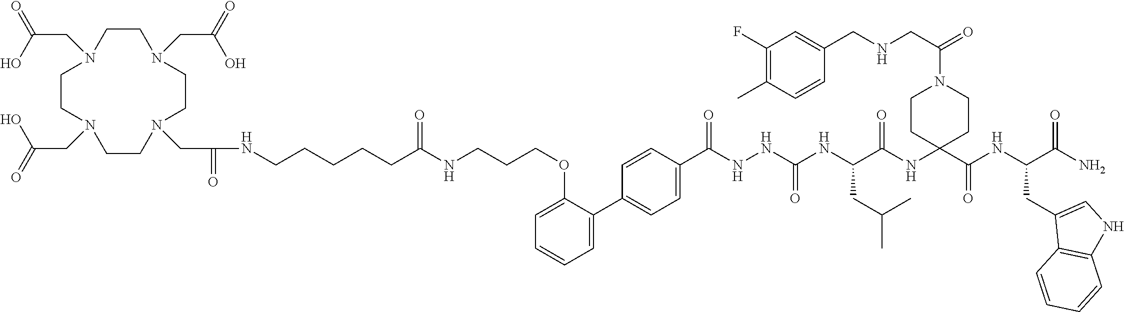 [SMILES: Cc1ccc(CNCC(=O)N2CCC(NC(=O)[C@H](CC(C)C)NC(=O)NNC(=O)c3ccc(-c4ccccc4OCCCNC(=O)CCCCCNC(=O)CN4CCN(CC(=O)O)CCN(CC(=O)O)CCN(CC(=O)O)CC4)cc3)(C(=O)N[C@@H](Cc3c[nH]c4ccccc34)C(N)=O)CC2)cc1F] |
| 298 | 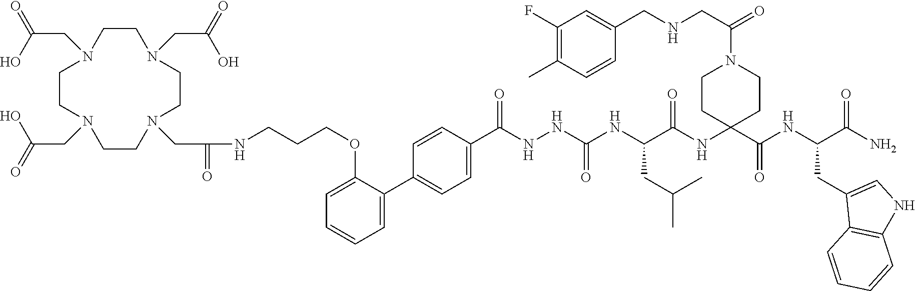 [SMILES: Cc1ccc(CNCC(=O)N2CCC(NC(=O)[C@H](CC(C)C)NC(=O)NNC(=O)c3ccc(-c4ccccc4OCCCNC(=O)CN4CCN(CC(=O)O)CCN(CC(=O)O)CCN(CC(=O)O)CC4)cc3)(C(=O)N[C@@H](Cc3c[nH]c4ccccc34)C(N)=O)CC2)cc1F] |
| 299 | SEQ ID NO: 681 |
| 300 | SEQ ID NO: 682 |
| 301 | SEQ ID NO: 683 |
| 302 | SEQ ID NO: 684 |
| 304 | SEQ ID NO: 686 |
| 305 | SEQ ID NO: 687 |
| 306 | SEQ ID NO: 688 |
| 307 | SEQ ID NO: 689 |
| 308 | SEQ ID NO: 690 |
| 309 | SEQ ID NO: 691 |
| 310 | SEQ ID NO: 692 |
| 311 | SEQ ID NO: 693 |
| 312 | SEQ ID NO: 694 |
| 313 | SEQ ID NO: 695 |
| 314 | SEQ ID NO: 696 |
| 315 | SEQ ID NO: 697 |
| 316 | SEQ ID NO: 698 |
| 317 | SEQ ID NO: 699 |
| 318 | SEQ ID NO: 700 |
| 319 | SEQ ID NO: 701 |
| 320 | SEQ ID NO: 702 |
| 321 | SEQ ID NO: 703 |
| 322 | SEQ ID NO: 704 |
| 323 | SEQ ID NO: 705 |
| 324 | SEQ ID NO: 706 |
| 325 | SEQ ID NO: 707 |
| 326 | SEQ ID NO: 708 |
| 327 | SEQ ID NO: 709 |
| 328 | SEQ ID NO: 710 |
| 329 | SEQ ID NO: 711 |
| 330 | SEQ ID NO: 712 |
| 331 | SEQ ID NO: 713 |
| 332 | SEQ ID NO: 714 |
| 333 | SEQ ID NO: 715 |
| 334 | SEQ ID NO: 716 |
| 335 | SEQ ID NO: 717 |
| 336 | SEQ ID NO: 718 |
| 337 | SEQ ID NO: 719 |
| 338 | SEQ ID NO: 720 |
| 339 | SEQ ID NO: 721 |
| 345 | SEQ ID NO: 727 |
| 346 | SEQ ID NO: 728 |
| 353 | SEQ ID NO: 735 |
| 354 | SEQ ID NO: 736 |
| 355 | SEQ ID NO: 737 |
| 356 | SEQ ID NO: 738 |
| 357 | SEQ ID NO: 739 |
| 358 | SEQ ID NO: 740 |
| 359 | SEQ ID NO: 741 |
| 360 | SEQ ID NO: 742 |
| 363 | SEQ ID NO: 745 |
| 364 | SEQ ID NO: 746 |
| 365 | SEQ ID NO: 747 |
| 366 | SEQ ID NO: 748 |
| 367 | SEQ ID NO: 749 |
| 368 | SEQ ID NO: 750 |
| 370 | SEQ ID NO: 752 |
| 371 | SEQ ID NO: 753 |
| 373 | SEQ ID NO: 755 |
| 374 | SEQ ID NO: 756 |
| 375 | SEQ ID NO: 757 |
| 376 | SEQ ID NO: 758 |
| 378 | SEQ ID NO: 759 |
| 379 | SEQ ID NO: 760 |
| 380 | SEQ ID NO: 761 |
| 381 | SEQ ID NO: 762 |
| 382 | SEQ ID NO: 763 |
| 383 | SEQ ID NO: 764 |
| 384 | SEQ ID NO: 765 |
| 386 | SEQ ID NO: 767 |
| 387 | SEQ ID NO: 768 |
| 388 | SEQ ID NO: 769 |
| 389 | SEQ ID NO: 770 |

In some embodiments, the compound of Formula (I) has a structure as shown in Table B-1, or a pharmaceutically acceptable salt thereof, wherein R^{a} is

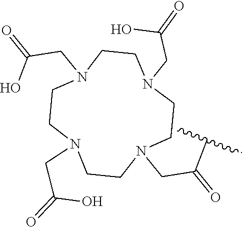 [SMILES: *C(=O)CN1CCN(CC(=O)O)CCN(CC(=O)O)CCN(CC(=O)O)CC1]

TABLE B-1

| Cmp. | |
|---|---|
| # | Structure |
| 40 | 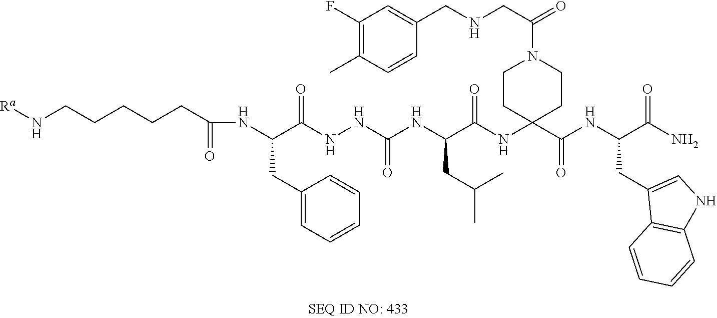 |
| 41 | 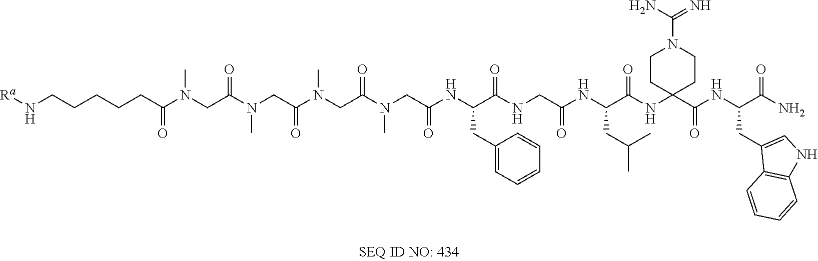 |
| 42 | 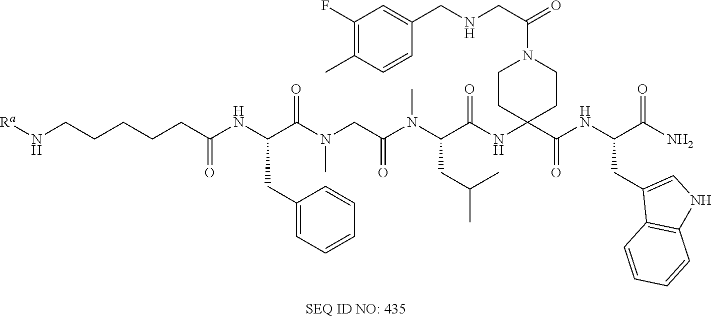 |
| 43 | 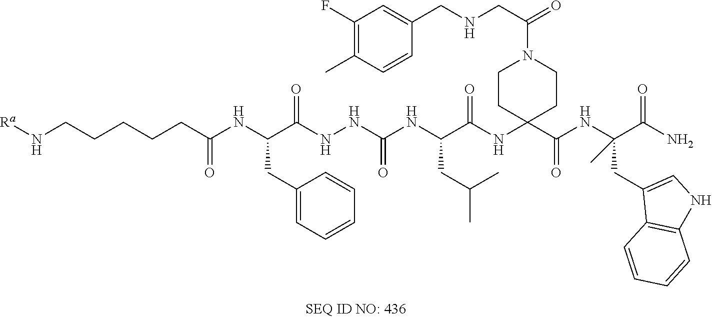 |
| 44 | 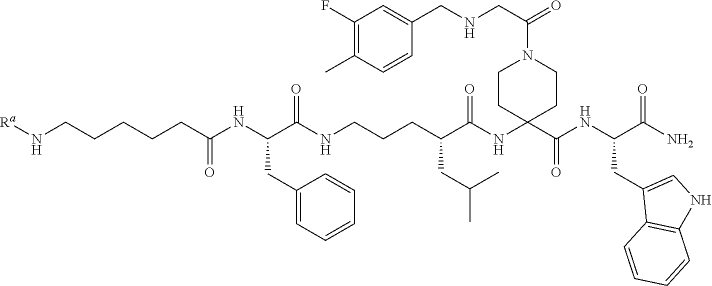 |
| 45 | 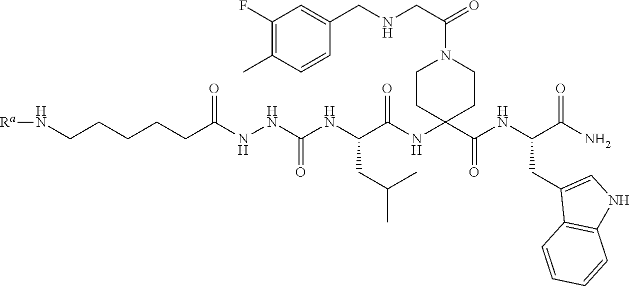 |
| 46 | 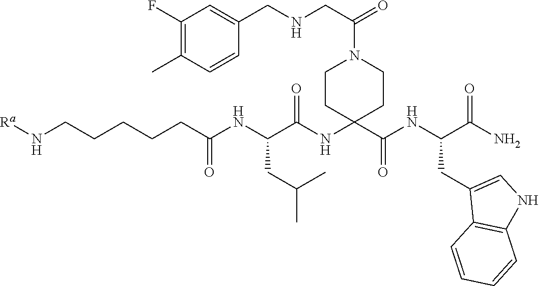 |
| 47 | 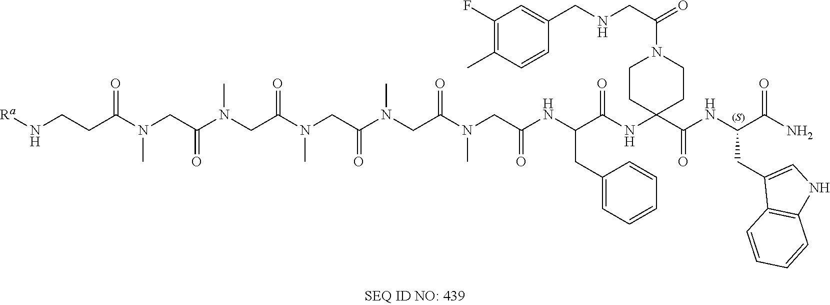 |
| 48 | 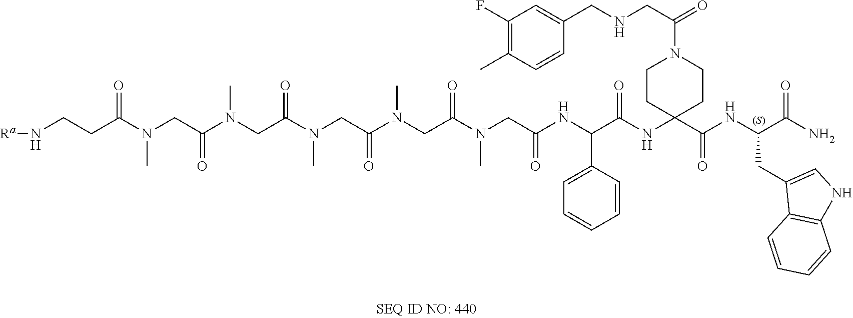 |
| 52 | 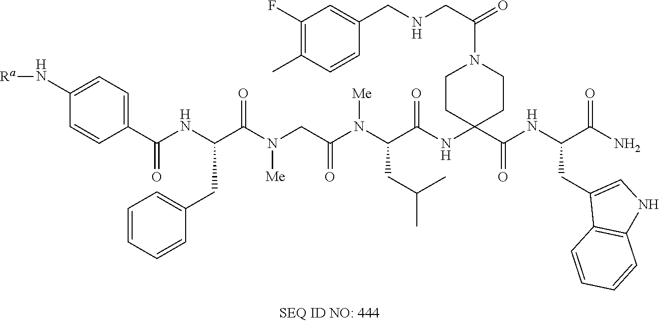 |
| 53 | 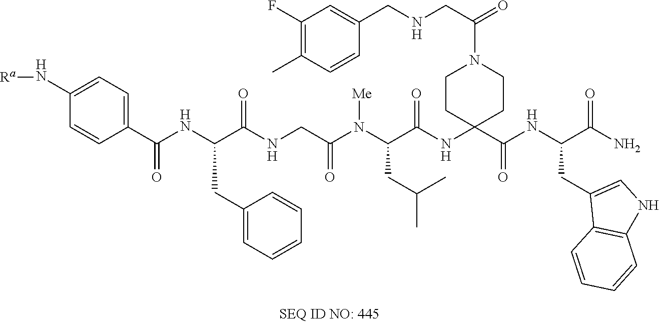 |
| 54 | 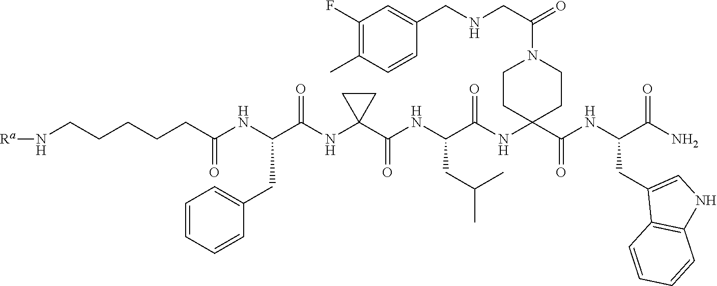 |
| 56 | 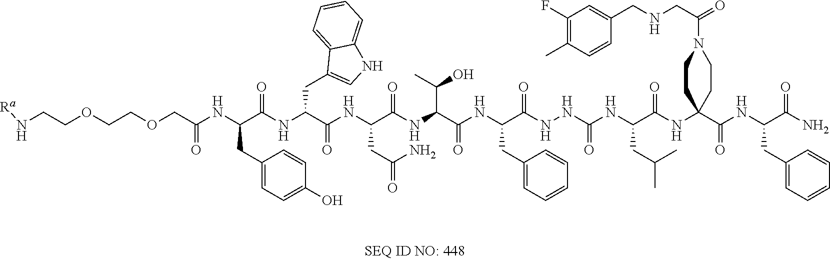 |
| 59 | 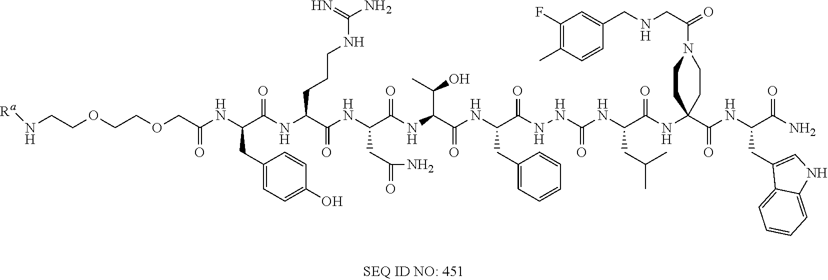 |
| 60 | 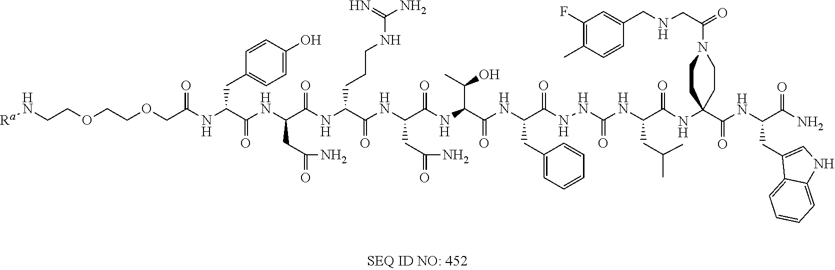 |
| 61 | 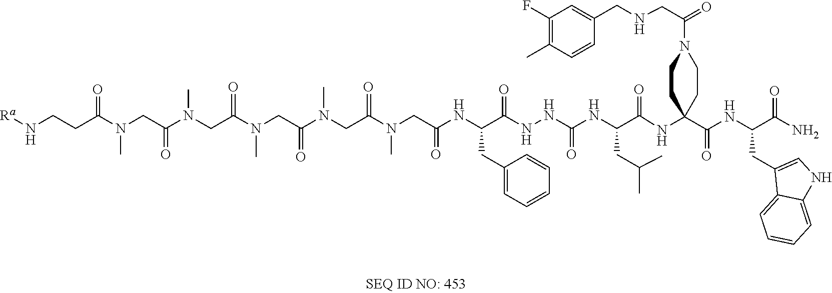 |
| 62 | 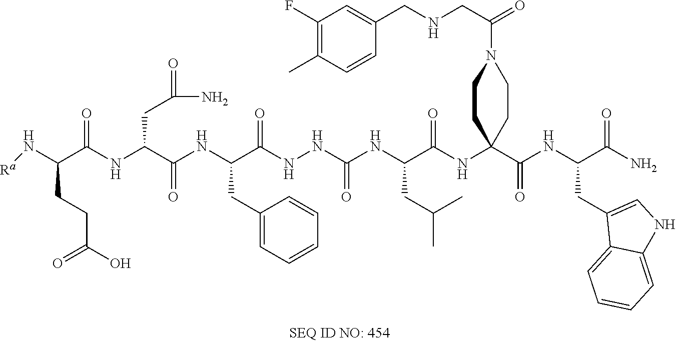 |
| 63 | 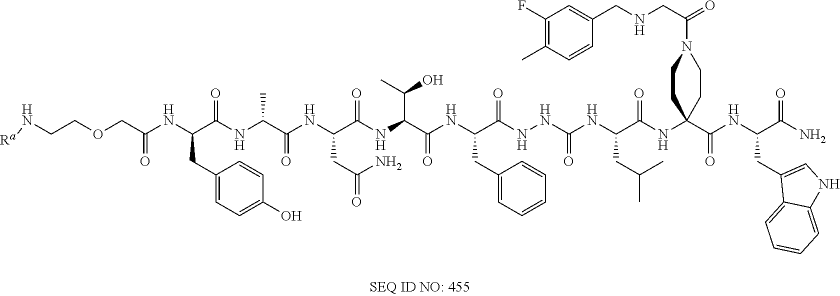 |
| 65 | 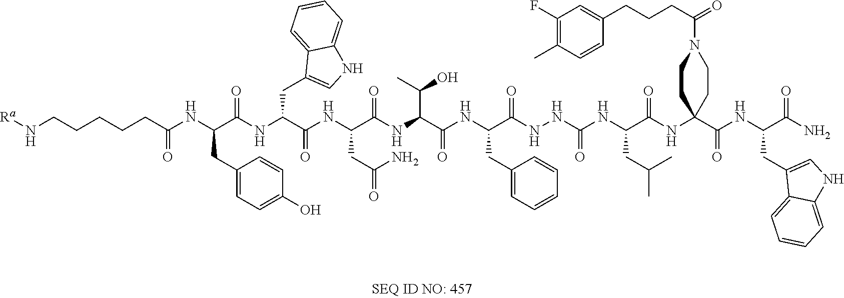 |
| 66 | 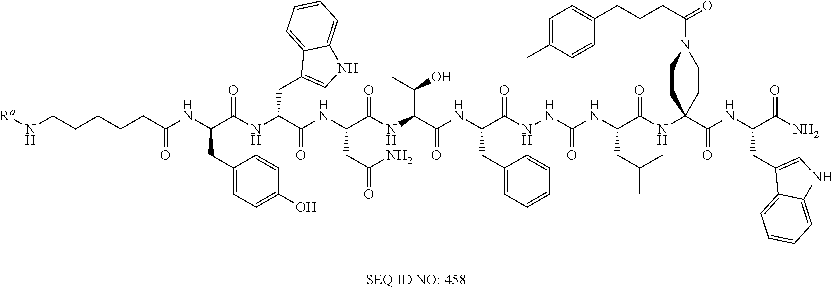 |
| 67 | 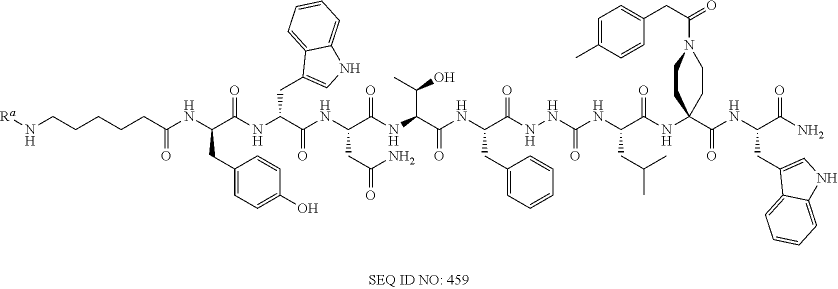 |
| 68 | 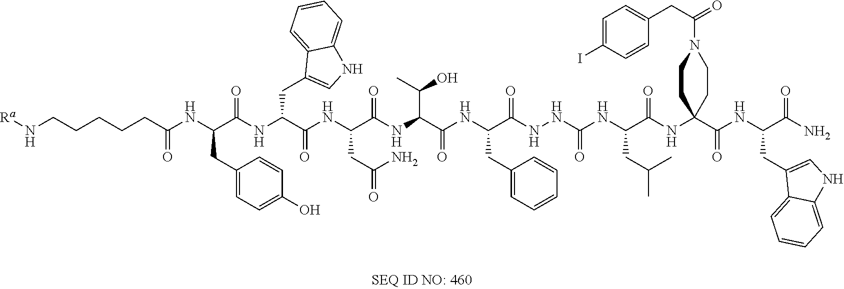 |
| 69 | 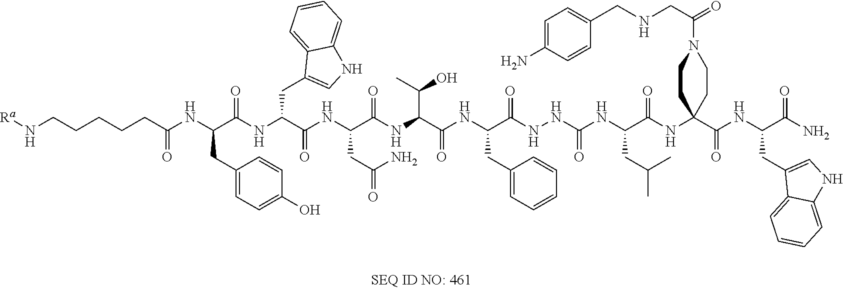 |
| 70 | 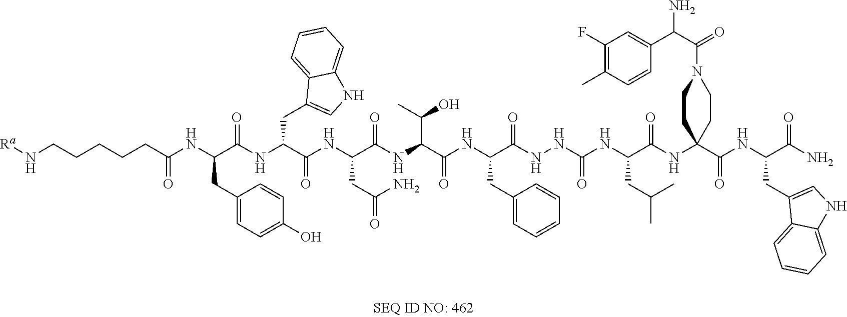 |
| 71 | 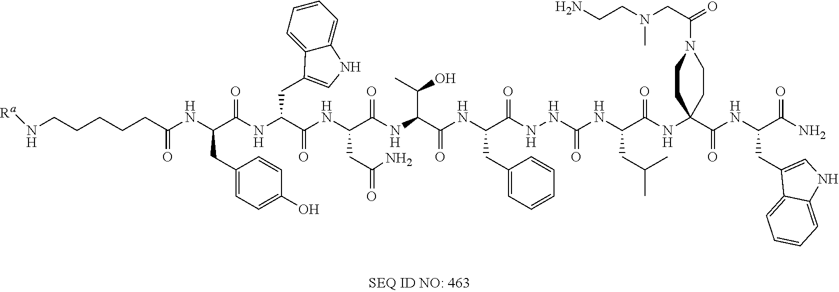 |
| 72 | 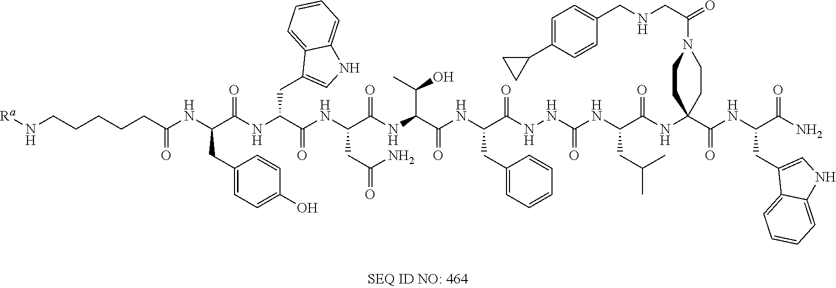 |
| 73 | 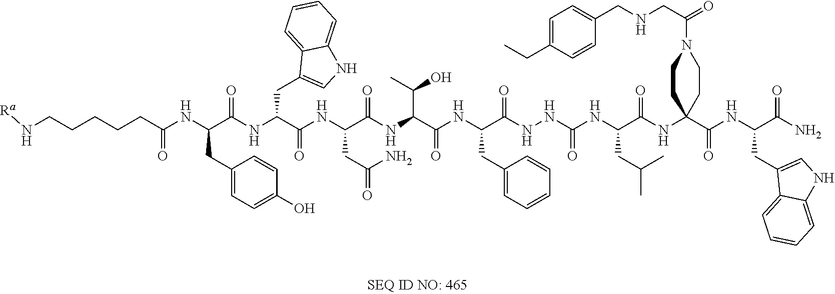 |
| 74 | 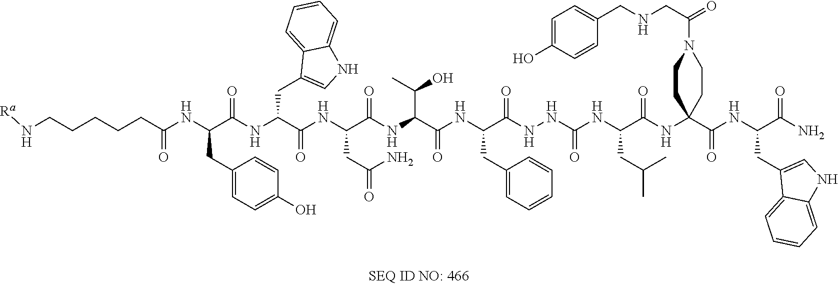 |
| 75 | 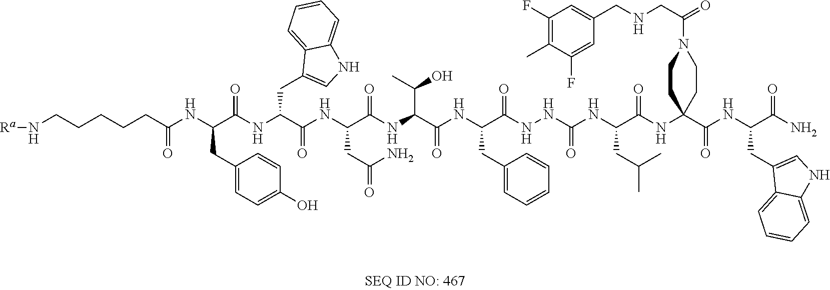 |
| 76 | 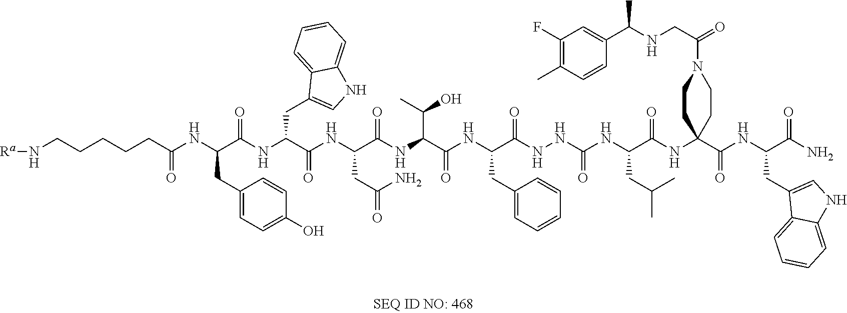 |
| 77 | 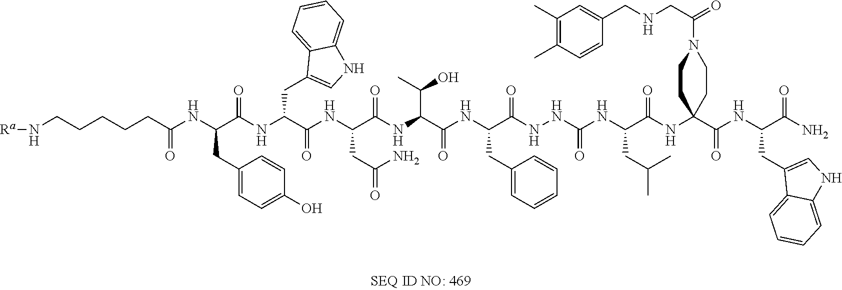 |
| 78 | 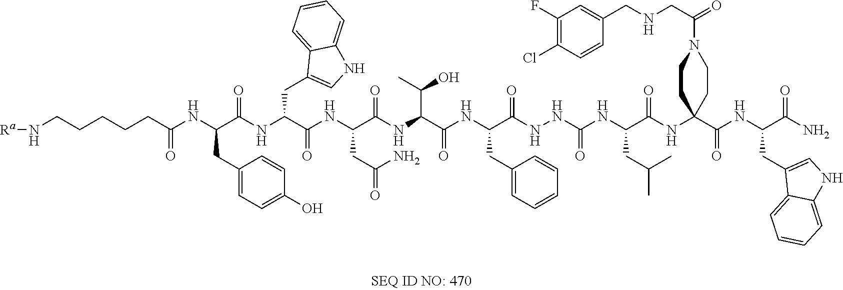 |
| 79 | 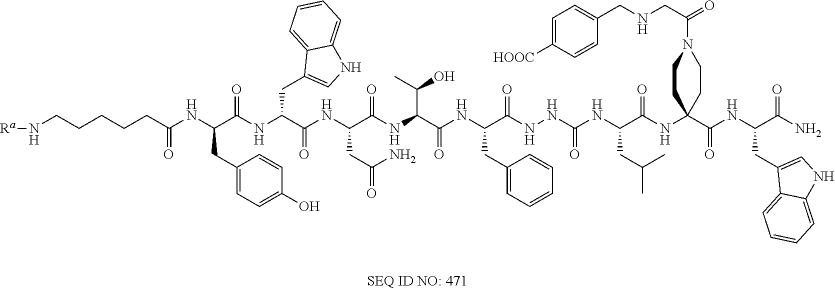 |
| 80 | 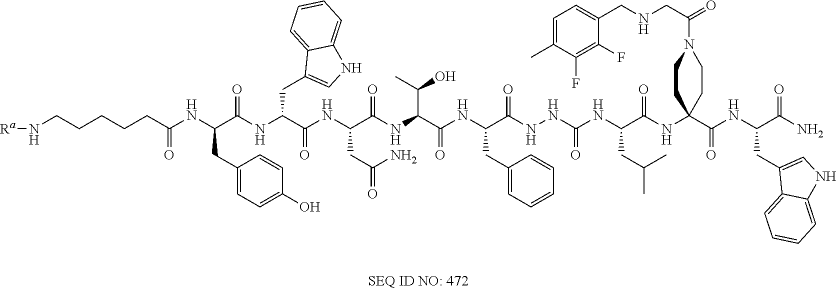 |
| 81 | 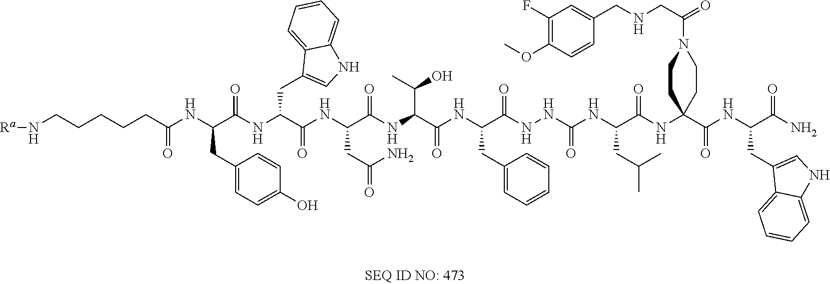 |
| 82 | 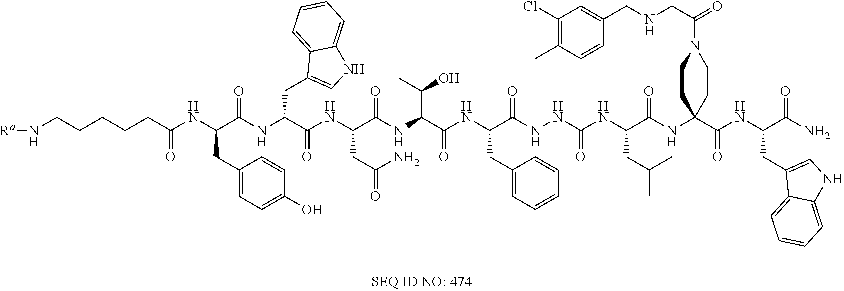 |
| 83 | 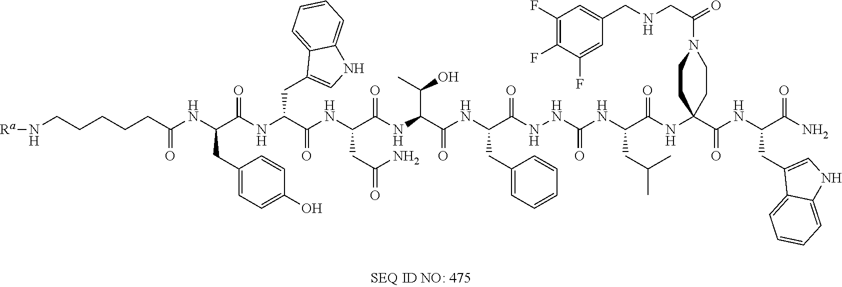 |
| 84 | 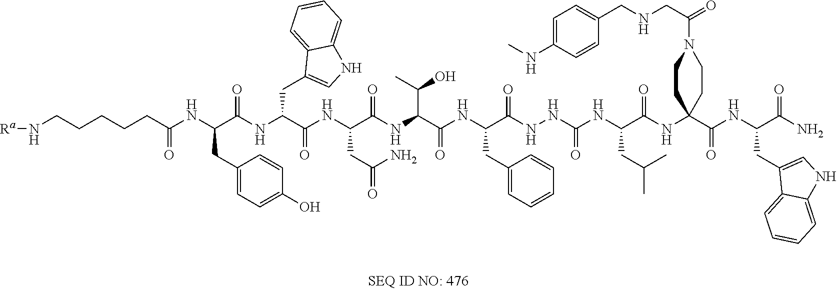 |
| 85 | 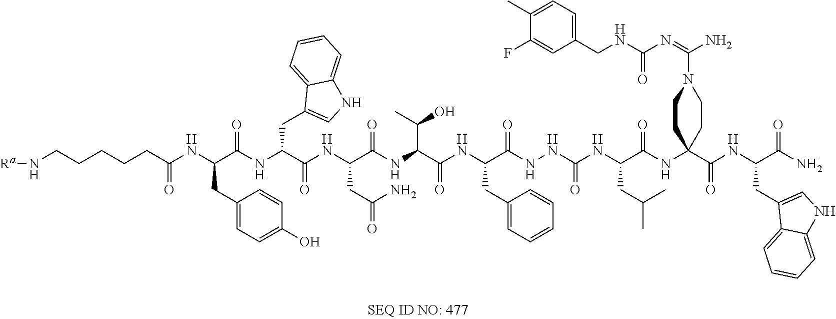 |
| 86 | 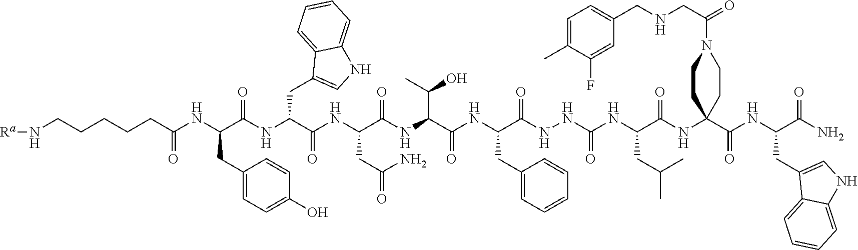 |
| | SEQ ID NO: 478 |
| 87 | 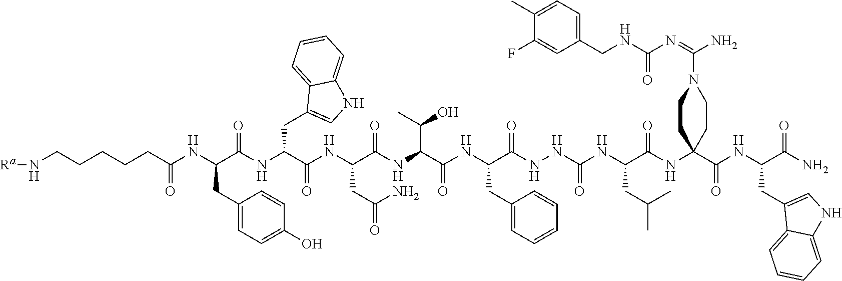 |
| | SEQ ID NO: 477 |
| 88 | 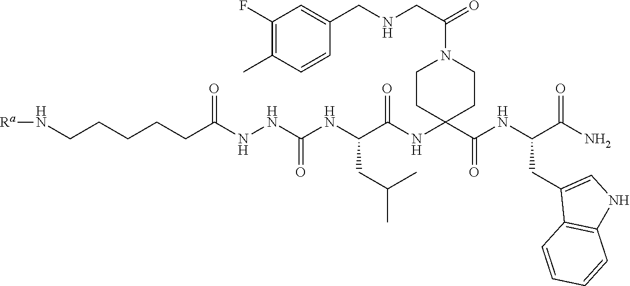 |
| 89 | 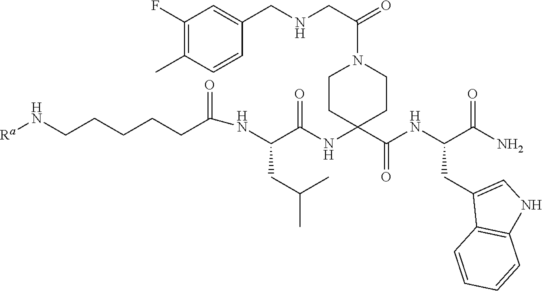 |
| 90 | 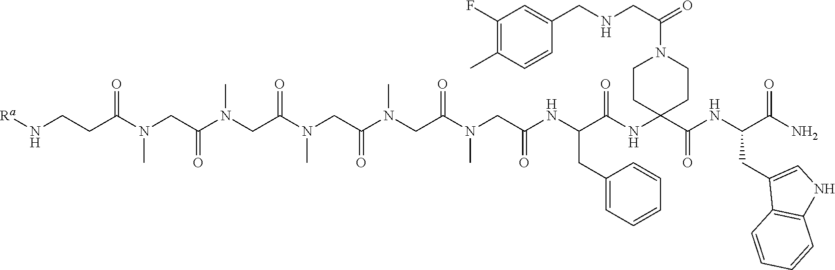 |
| | SEQ ID NO: 439 |
| 92 | 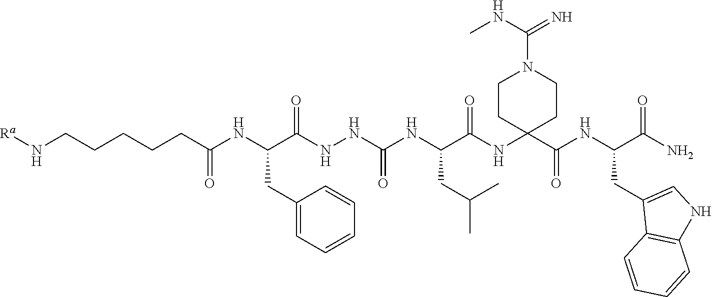 |
| | SEQ ID NO: 480 |
| 93 | 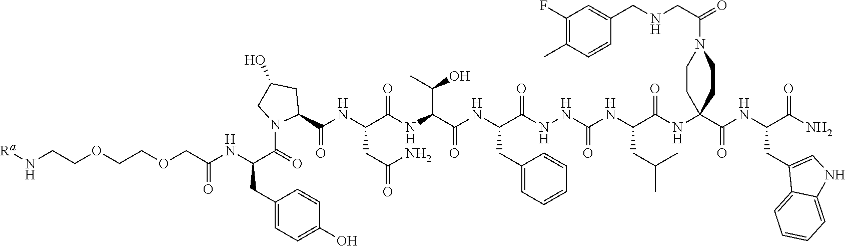 |
| | SEQ ID NO: 481 |
| 94 | 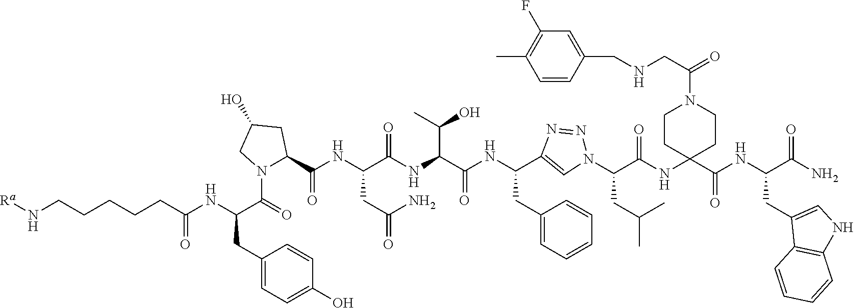 |
| | SEQ ID NO: 482 |
| 95 | 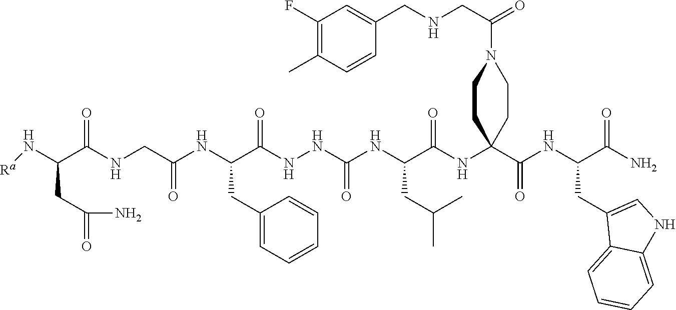 |
| | SEQ ID NO: 483 |
| 96 | 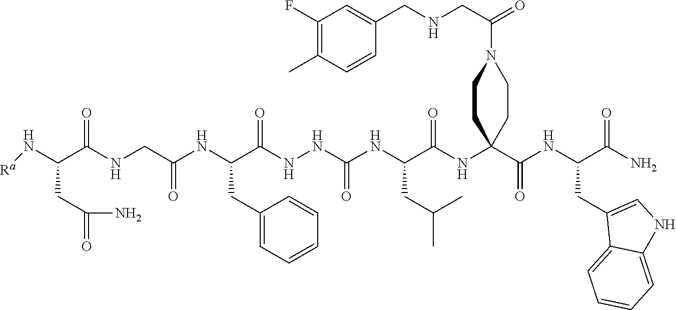 |
| | SEQ ID NO: 484 |
| 97 | 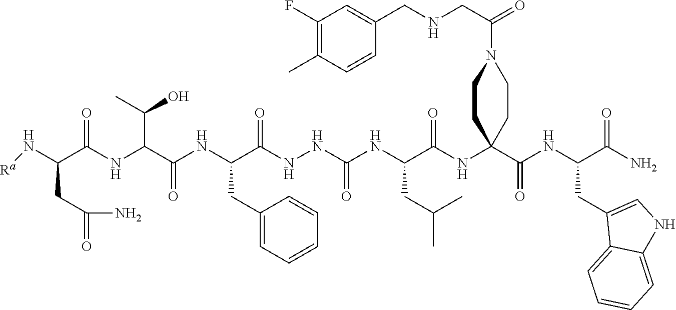 |
| | SEQ ID NO: 485 |
| 98 | 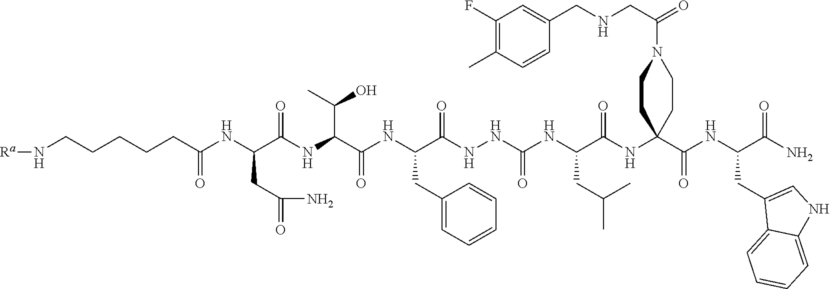 |
| | SEQ ID NO: 486 |
| 99 | 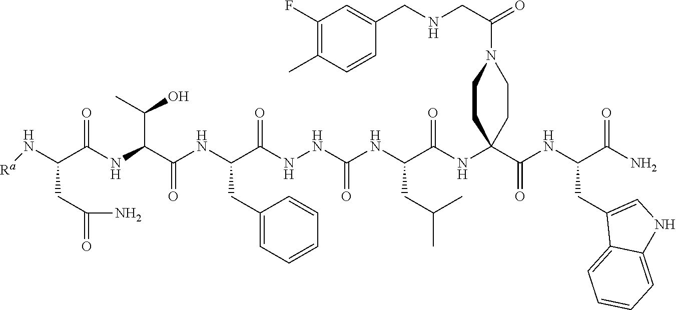 |
| | SEQ ID NO: 487 |
| 100 | 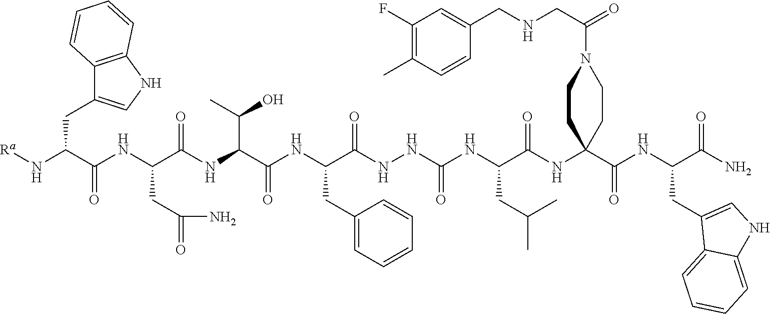 |
| | SEQ ID NO: 488 |
| 101 | 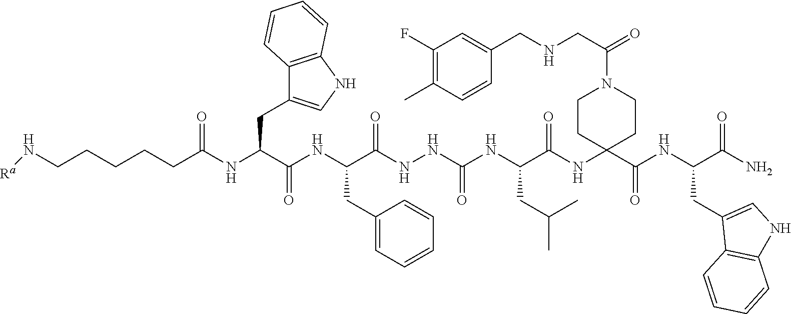 |
| | SEQ ID NO: 489 |
| 102 | 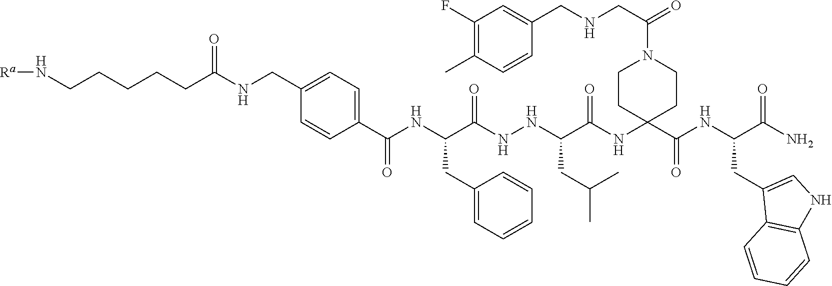 |
| | SEQ ID NO: 415 |
| 103 | 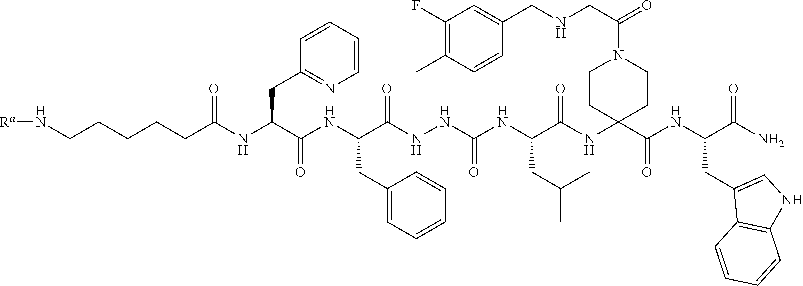 |
| | SEQ ID NO: 490 |
| 112 | 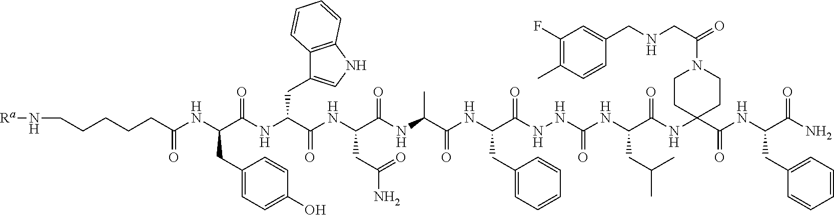 |
| | SEQ ID NO: 499 |
| 122 | 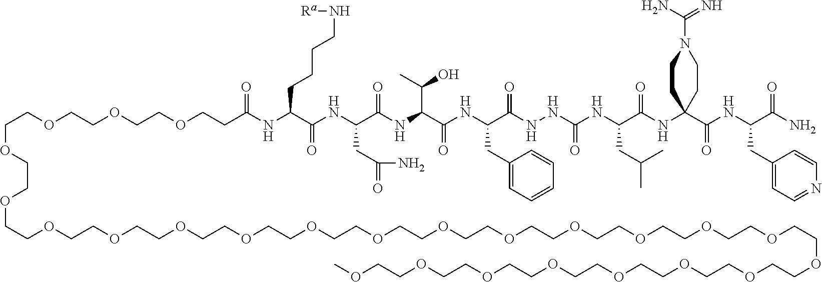 |
| | SEQ ID NO: 509 |
| 143 | 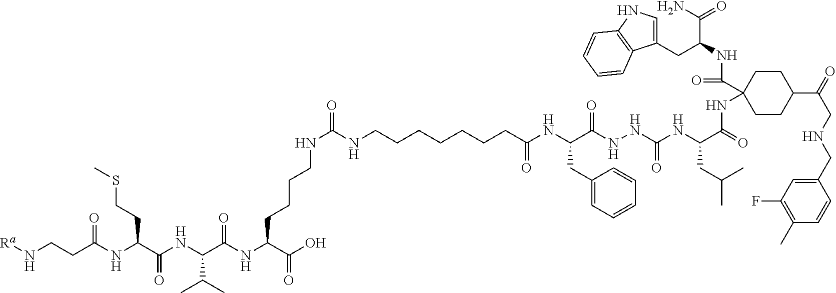 |
| | SEQ ID NO: 524 |
| 144 | 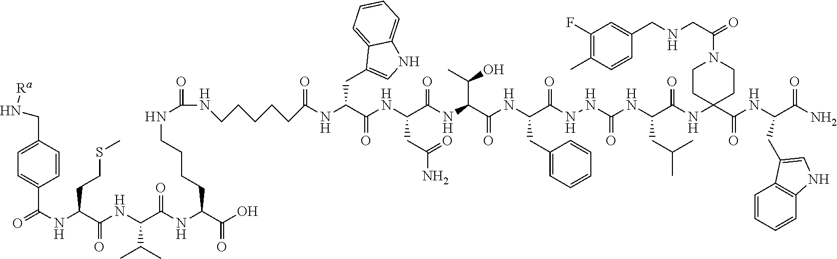 |
| | SEQ ID NO: 525 |
| 145 | 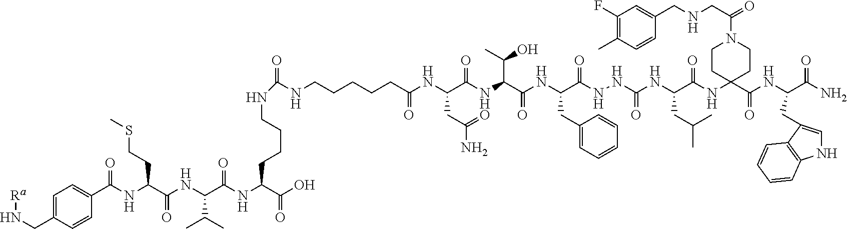 |
| | SEQ ID NO: 526 |
| 150 | 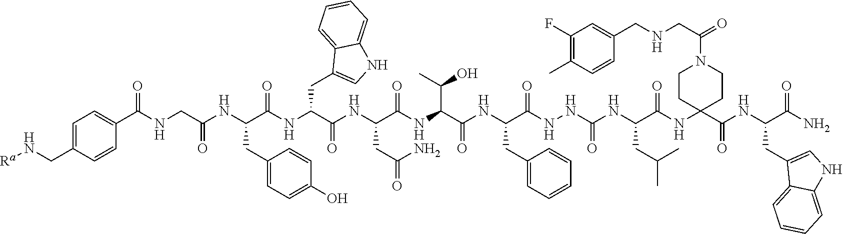 |
| | SEQ ID NO: 531 |
| 152 | 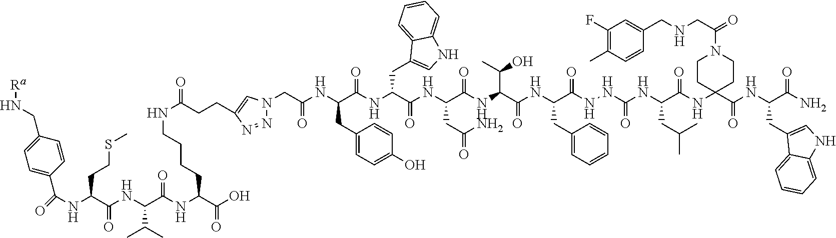 |
| | SEQ ID NO: 533 |
| 153 | 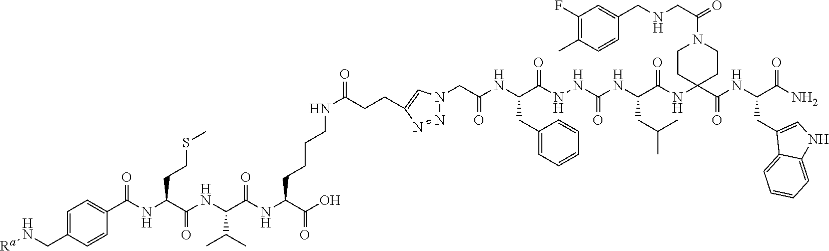 |
| | SEQ ID NO: 534 |
| 154 | 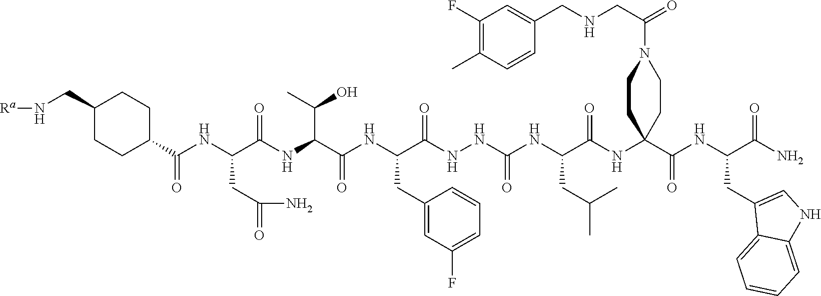 |
| | SEQ ID NO: 535 |
| 155 | 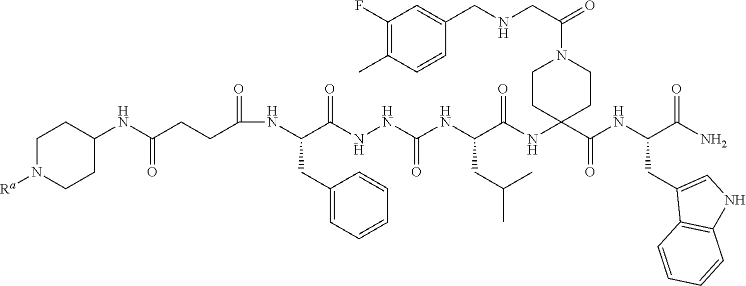 |
| | SEQ ID NO: 536 |
| 157 | 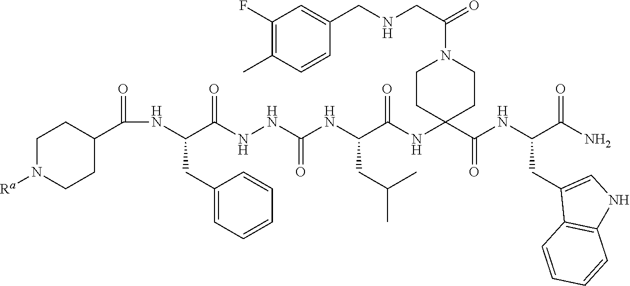 |
| | SEQ ID NO: 538 |
| 162 | 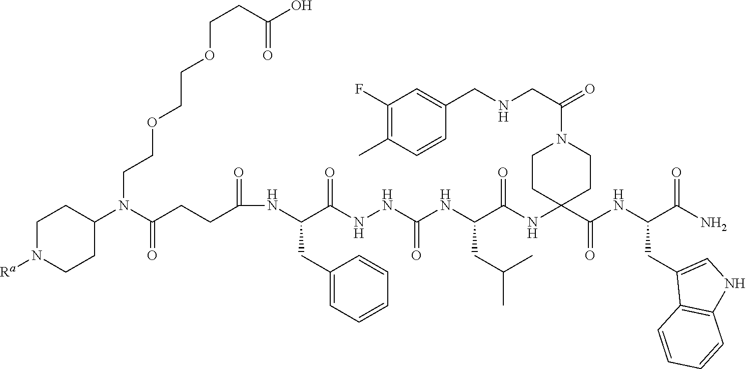 |
| | SEQ ID NO: 543 |
| 163 | 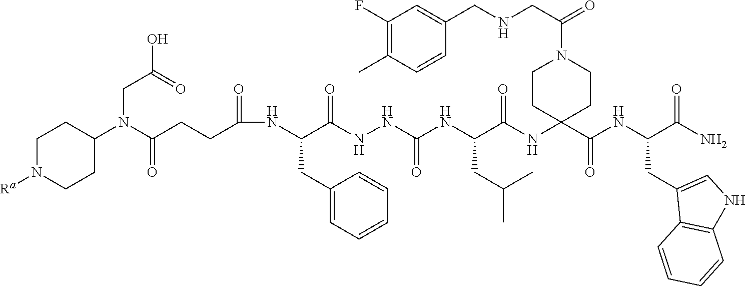 |
| | SEQ ID NO: 544 |
| 172 | 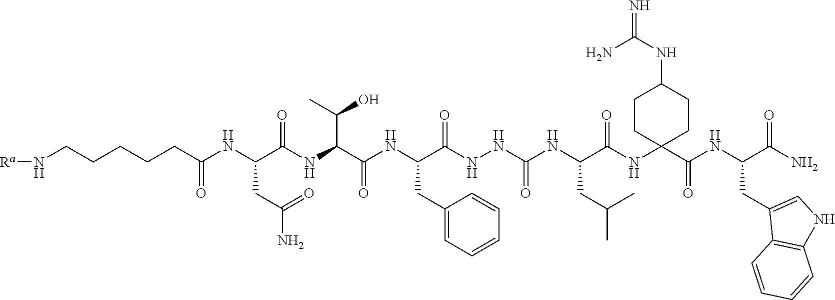 |
| | SEQ ID NO: 800 |
| 173 | 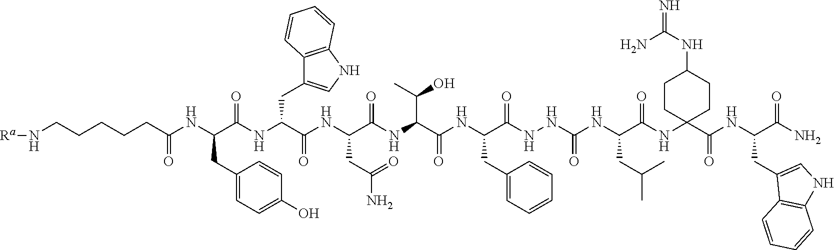 |
| | SEQ ID NO: 801 |
| 174 | 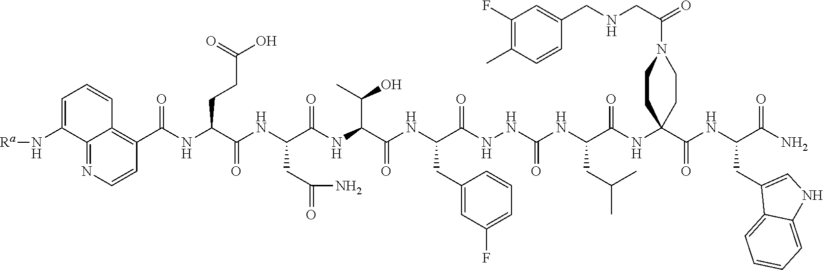 |
| | SEQ ID NO: 553 |
| 175 | 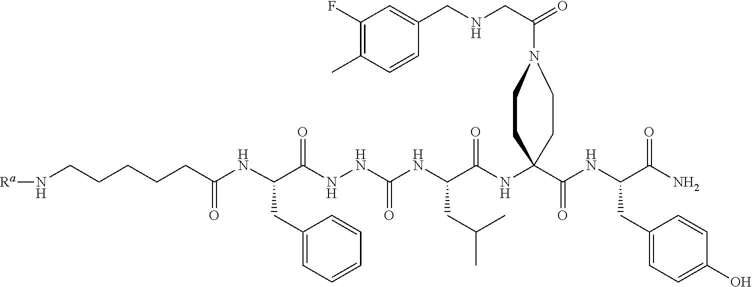 |
| | SEQ ID NO: 554 |
| 177 | 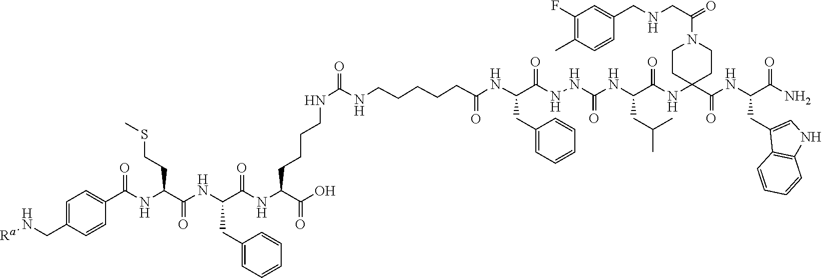 |
| | SEQ ID NO: 556 |
| 178 | 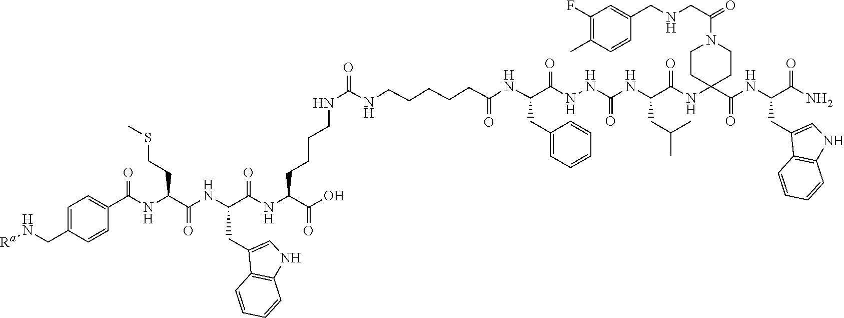 |
| | SEQ ID NO: 557 |
| 179 | 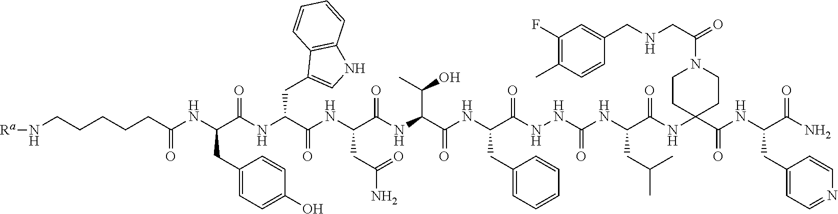 |
| | SEQ ID NO: 558 |
| 180 | 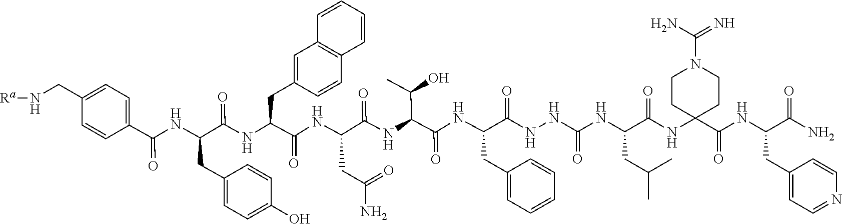 |
| | SEQ ID NO: 559 |
| 190 | 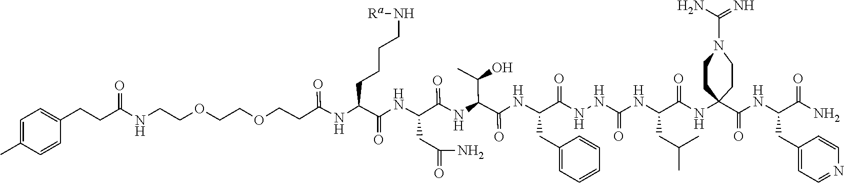 |
| | SEQ ID NO: 569 |
| 191 | 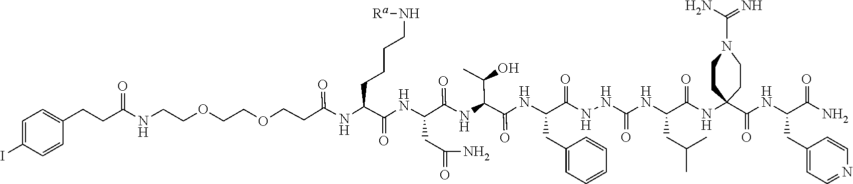 |
| | SEQ ID NO: 570 |
| 194 | 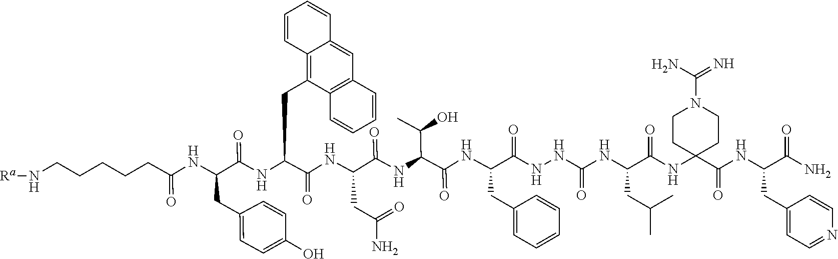 |
| | SEQ ID NO: 574 |
| 207 | 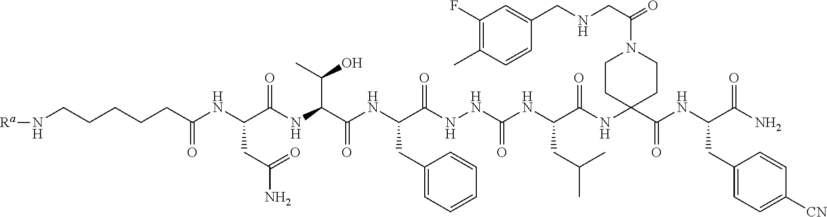 |
| | SEQ ID NO: 588 |
| 208 | 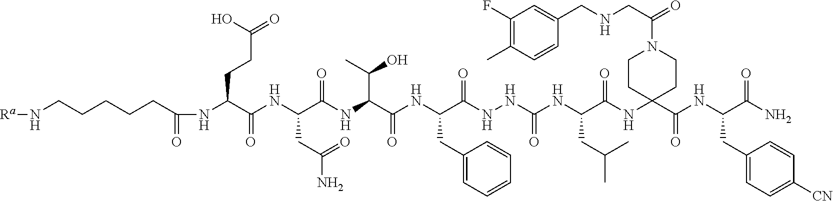 |
| | SEQ ID NO: 589 |
| 209 | 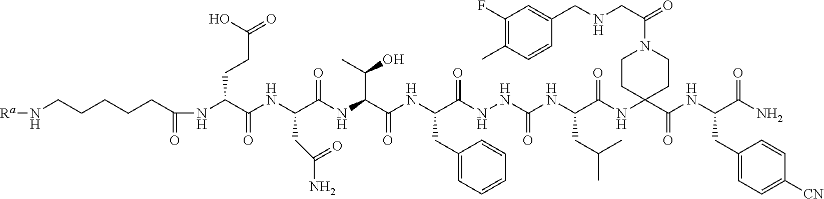 |
| | SEQ ID NO: 590 |
| 210 | 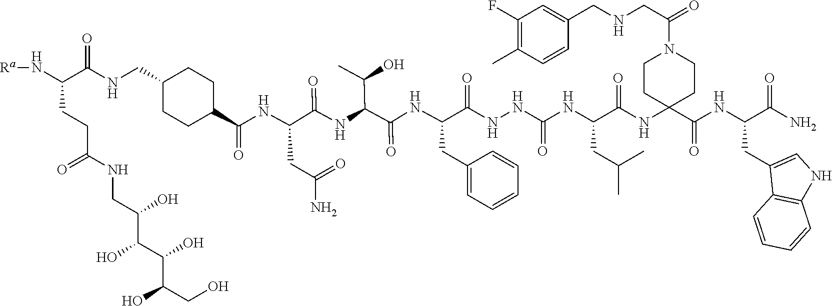 |
| | SEQ ID NO: 591 |
| 211 | 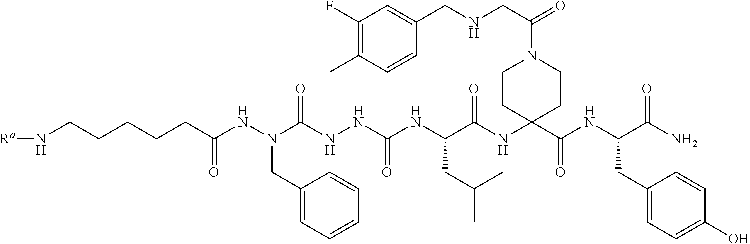 |
| 212 | 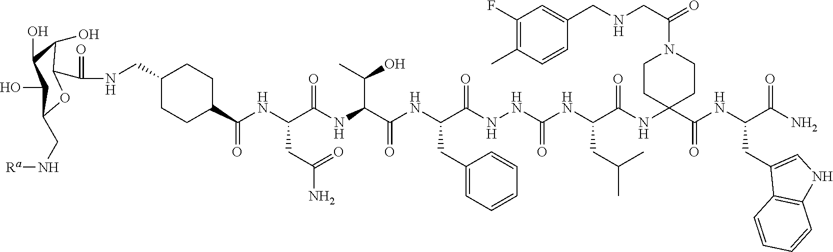 |
| | SEQ ID NO: 593 |
| 219 | 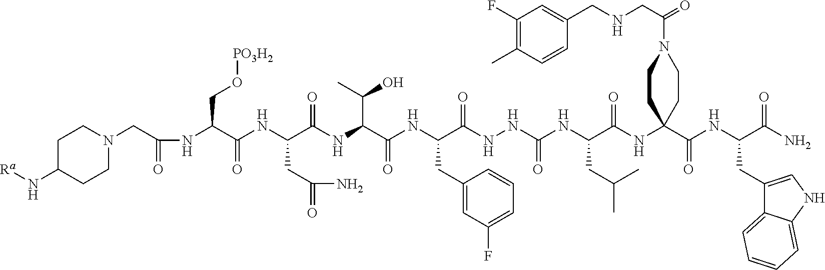 |
| | SEQ ID NO: 600 |
| 220 | 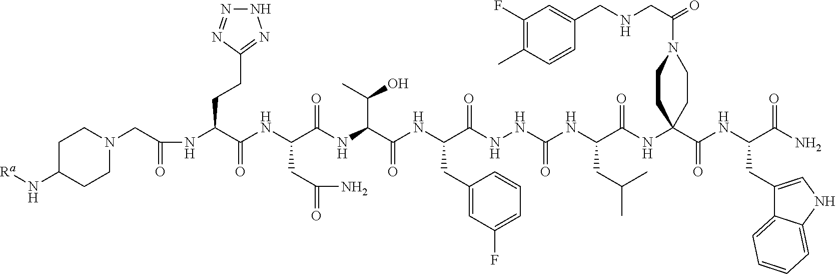 |
| | SEQ ID NO: 601 |
| 224 | 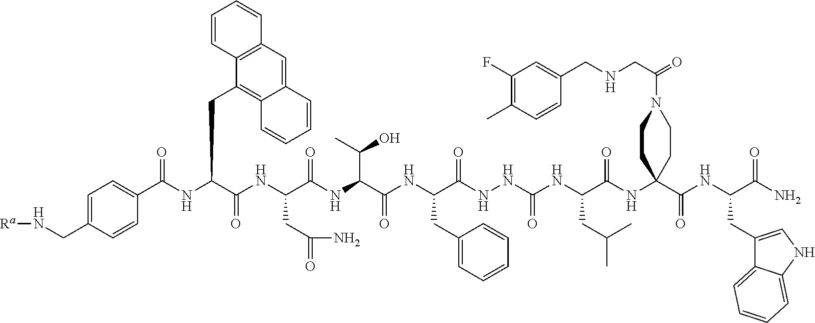 |
| | SEQ ID NO: 605 |
| 225 | 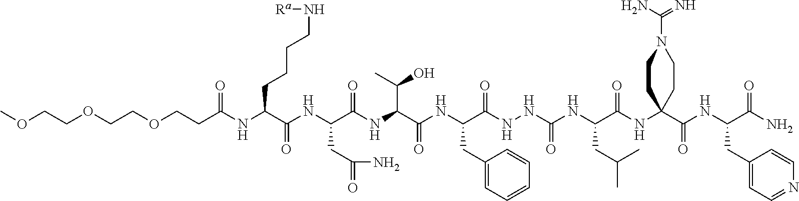 |
| | SEQ ID NO: 606 |
| 227 | 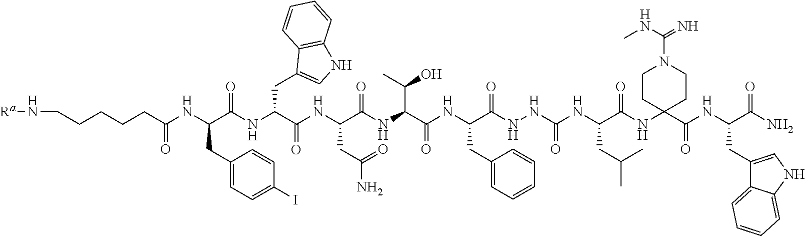 |
| | SEQ ID NO: 608 |
| 229 | 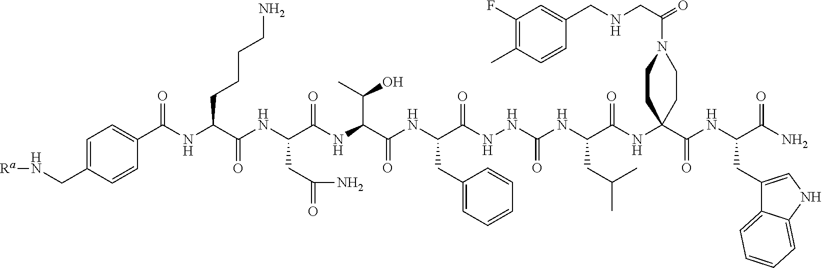 |
| | SEQ ID NO: 610 |
| 243 | 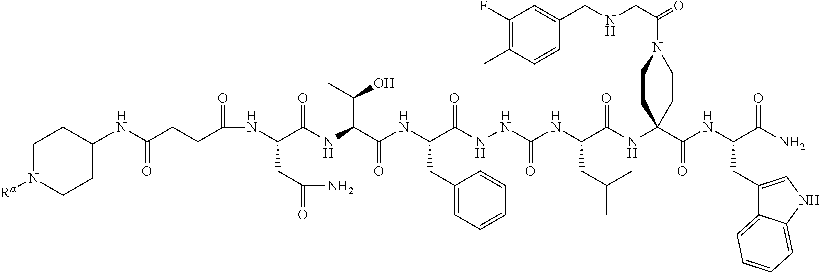 |
| | SEQ ID NO: 624 |
| 257 | 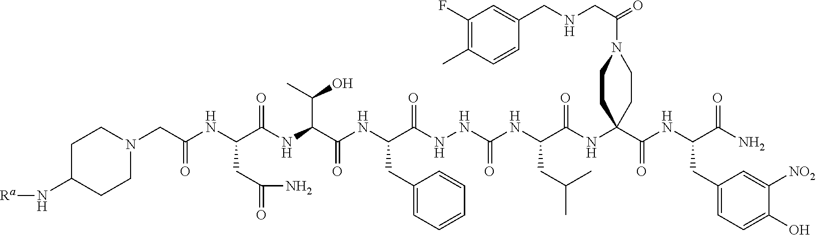 |
| | SEQ ID NO: 638 |
| 262 | 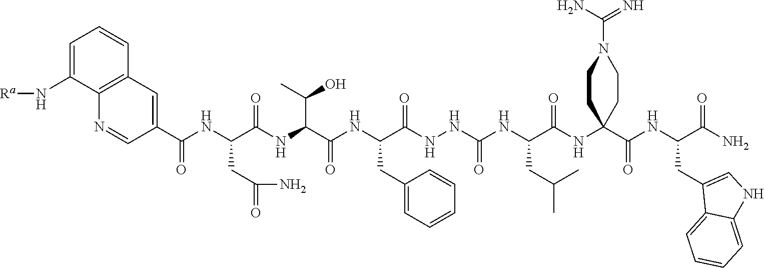 |
| | SEQ ID NO: 643 |
| 267 | 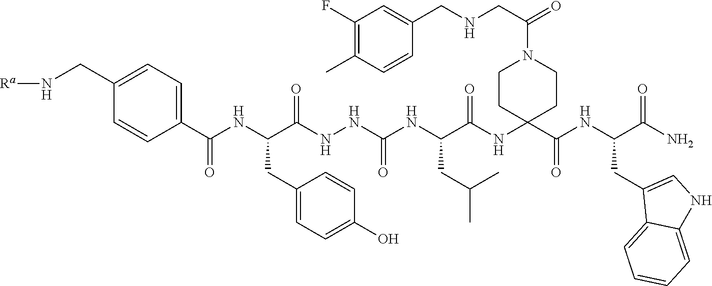 |
| | SEQ ID NO: 648 |
| 268 | 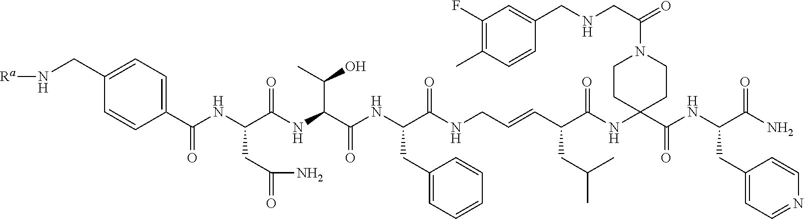 |
| 274 | 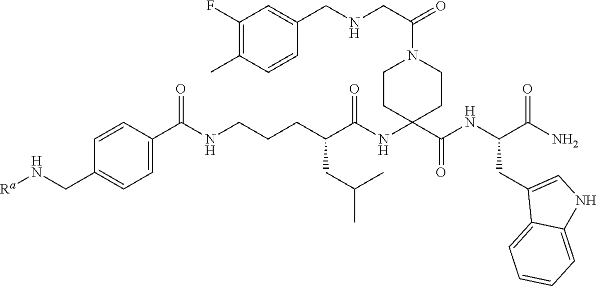 |
| 340 | 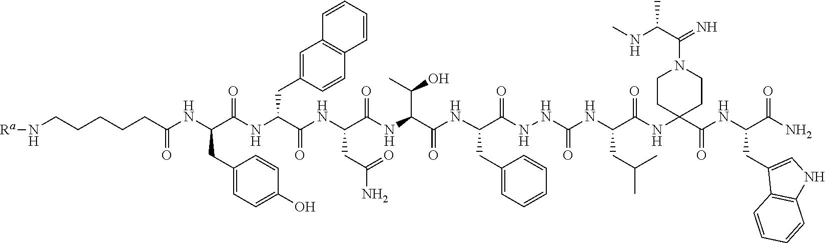 |
| | SEQ ID NO: 722 |
| 341 | 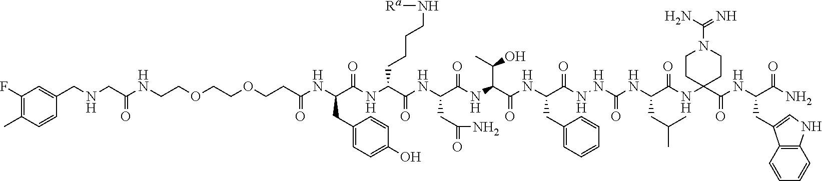 |
| | SEQ ID NO: 723 |
| 342 | 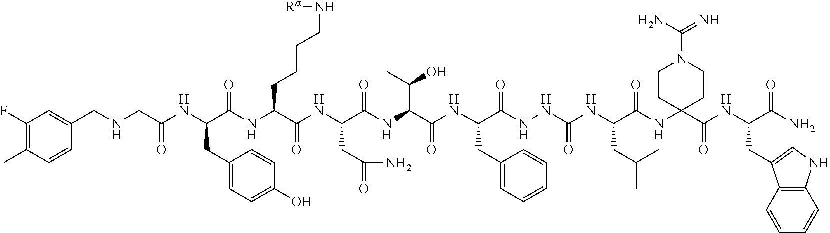 |
| | SEQ ID NO: 724 |
| 343 | 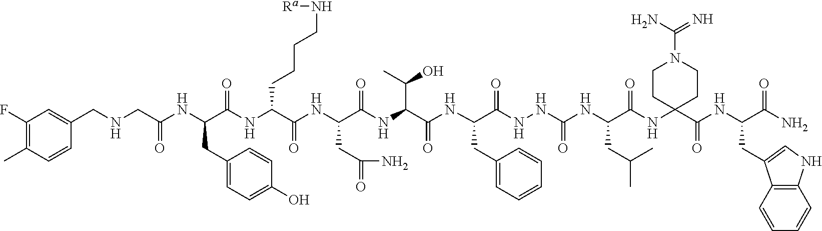 |
| | SEQ ID NO: 725 |
| 344 | 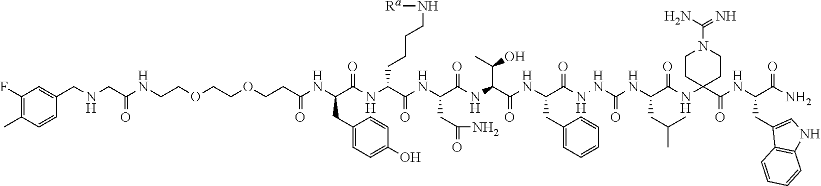 |
| | SEQ ID NO: 726 |
| 347 | 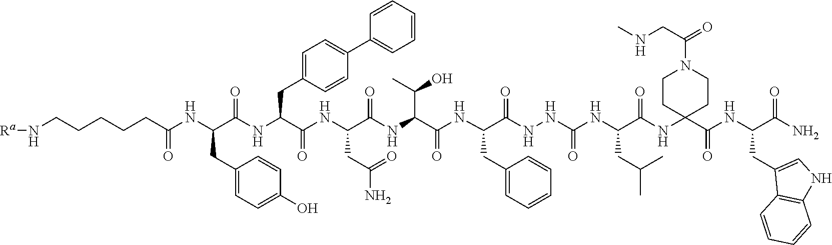 |
| | SEQ ID NO: 729 |
| 348 | 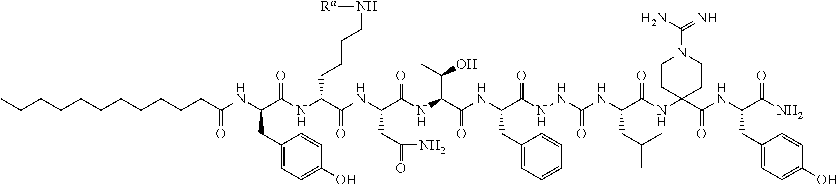 |
| | SEQ ID NO: 730 |
| 349 | 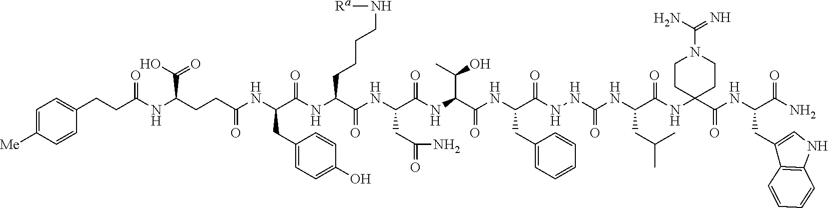 |
| | SEQ ID NO: 731 |
| 350 | 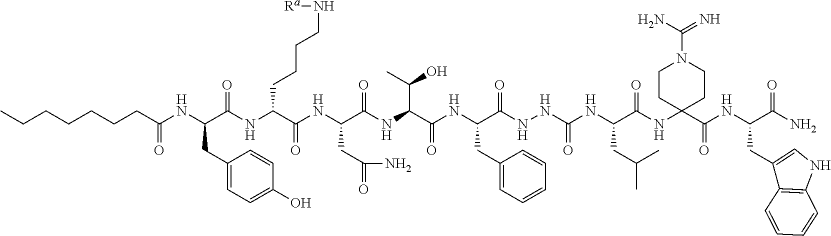 |
| | SEQ ID NO: 732 |
| 351 | 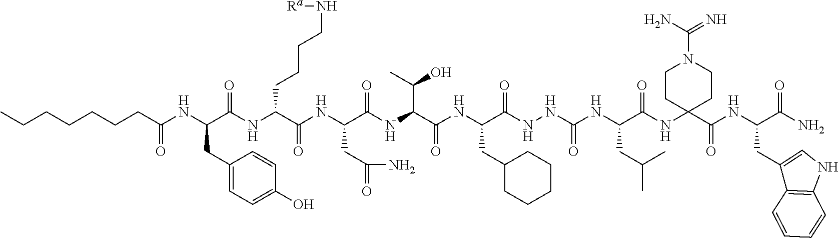 |
| | SEQ ID NO: 733 |
| 352 | 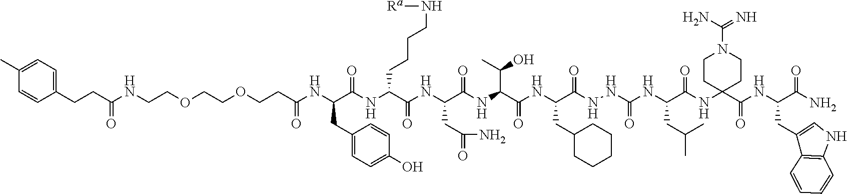 |
| | SEQ ID NO: 734 |
| 361 | 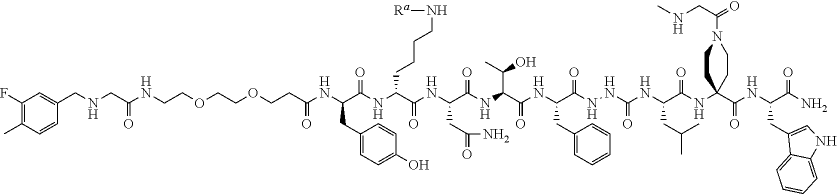 |
| | SEQ ID NO: 743 |
| 362 | 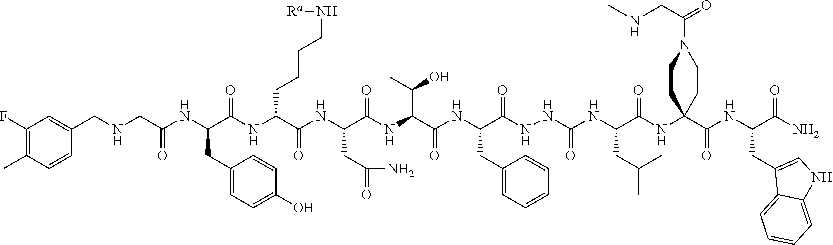 |
| | SEQ ID NO: 744 |
| 369 | 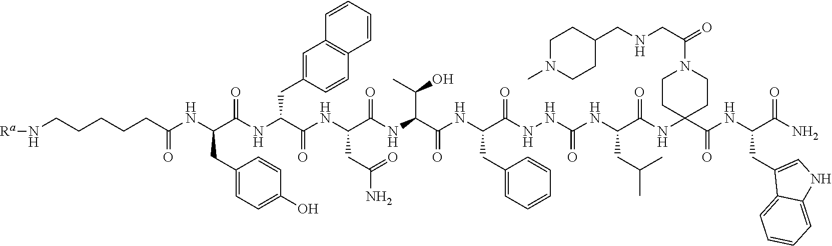 |
| | SEQ ID NO: 751 |
| 372 | 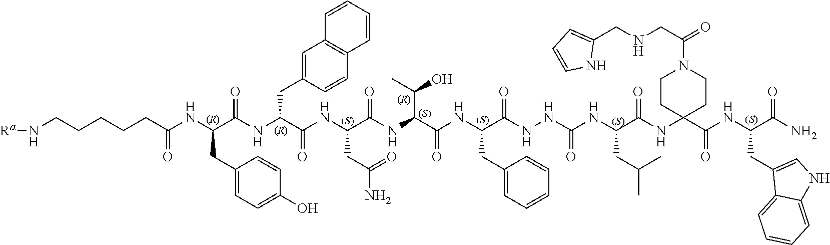 |
| | SEQ ID NO: 754 |
| 385 | 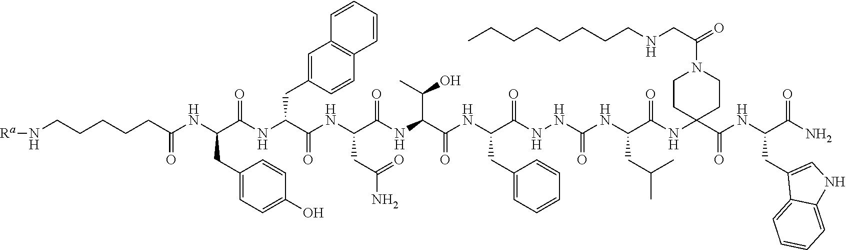 |
| | SEQ ID NO: 766 |

In some embodiments, the compound of Formula MI is compound 1, a pharmaceutically acceptable salt thereof, or radionuclide complex thereof; compound 1-In, a pharmaceutically acceptable salt thereof, compound 1-Lu, a pharmaceutically acceptable salt thereof; compound 1-Ga, a pharmaceutically acceptable salt thereof, compound 2, a pharmaceutically acceptable salt thereof, or a radionuclide complex thereof; compound 2-In, a pharmaceutically acceptable salt thereof, or a radionuclide complex thereof; compound 3, a pharmaceutically acceptable salt thereof, or a radionuclide complex thereof, compound 4, a pharmaceutically acceptable salt thereof, or a radionuclide complex thereof; compound 5, a pharmaceutically acceptable salt thereof, or a radionuclide complex thereof; compound 6, a pharmaceutically acceptable salt thereof, or a radionuclide complex thereof; compound 6-In, a pharmaceutically acceptable salt thereof, or a radionuclide complex thereof; compound 7, a pharmaceutically acceptable salt thereof, or a radionuclide complex thereof; compound 8, a pharmaceutically acceptable salt thereof, or a radionuclide complex thereof: compound 9, a pharmaceutically acceptable salt thereof, or a radionuclide complex thereof; compound 10, a pharmaceutically acceptable salt thereof, or a radionuclide complex thereof; compound 11, a pharmaceutically acceptable salt thereof, or a radionuclide complex thereof; compound 12, a pharmaceutically acceptable salt thereof, or a radionuclide complex thereof; compound 13, a pharmaceutically acceptable salt thereof, or a radionuclide complex thereof; compound 14, a pharmaceutically acceptable salt thereof, or a radionuclide complex thereof; compound 15, a pharmaceutically acceptable salt thereof, or a radionuclide complex thereof; compound 16, a pharmaceutically acceptable salt thereof, or a radionuclide complex thereof; compound 17, a pharmaceutically acceptable salt thereof, or a radionuclide complex thereof; compound 18, a pharmaceutically acceptable salt thereof, or a radionuclide complex thereof; compound 19, a pharmaceutically acceptable salt thereof, or a radionuclide complex thereof; compound 20, a pharmaceutically acceptable salt thereof, or a radionuclide complex thereof; compound 21, a pharmaceutically acceptable salt thereof, or a radionuclide complex thereof; compound 22, a pharmaceutically acceptable salt thereof, or a radionuclide complex thereof; compound 23, a pharmaceutically acceptable salt thereof, or a radionuclide complex thereof; compound 24, a pharmaceutically acceptable salt thereof, or a radionuclide complex thereof; compound 25, a pharmaceutically acceptable salt thereof, or a radionuclide complex thereof; compound 26, a pharmaceutically acceptable salt thereof, or a radionuclide complex thereof; compound 27, a pharmaceutically acceptable salt thereof, or a radionuclide complex thereof; compound 28, a pharmaceutically acceptable salt thereof, or a radionuclide complex thereof; compound 29, a pharmaceutically acceptable salt thereof, or a radionuclide complex thereof; compound 30, a pharmaceutically acceptable salt thereof, or a radionuclide complex thereof; compound 31, a pharmaceutically acceptable salt thereof, or a radionuclide complex thereof; compound 32, a pharmaceutically acceptable salt thereof, or a radionuclide complex thereof: compound 33, a pharmaceutically acceptable salt thereof, or a radionuclide complex thereof; compound 34, a pharmaceutically acceptable salt thereof, or a radionuclide complex thereof; compound 35, a pharmaceutically acceptable salt thereof, or a radionuclide complex thereof; compound 36, a pharmaceutically acceptable salt thereof, or a radionuclide complex thereof; compound 37, a pharmaceutically acceptable salt thereof, or a radionuclide complex thereof; compound 38, a pharmaceutically acceptable salt thereof, or a radionuclide complex thereof; compound 39, a pharmaceutically acceptable salt thereof, or a radionuclide complex thereof; compound 40, a pharmaceutically acceptable salt thereof, or a radionuclide complex thereof, compound 41, a pharmaceutically acceptable salt thereof, or a radionuclide complex thereof; compound 42, a pharmaceutically acceptable salt thereof, or a radionuclide complex thereof; compound 43, a pharmaceutically acceptable salt thereof, or a radionuclide complex thereof; compound 44, a pharmaceutically acceptable salt thereof, or a radionuclide complex thereof; compound 45, a pharmaceutically acceptable salt thereof, or a radionuclide complex thereof; compound 46, a pharmaceutically acceptable salt thereof, or a radionuclide complex thereof; compound 47, a pharmaceutically acceptable salt thereof, or a radionuclide complex thereof; compound 48, a pharmaceutically acceptable salt thereof, or a radionuclide complex thereof; compound 49, a pharmaceutically acceptable salt thereof, or a radionuclide complex thereof; compound 50, a pharmaceutically acceptable salt thereof, or a radionuclide complex thereof; compound 51, a pharmaceutically acceptable salt thereof, or a radionuclide complex thereof; compound 52, a pharmaceutically acceptable salt thereof, or a radionuclide complex thereof; compound 53, a pharmaceutically acceptable salt thereof, or a radionuclide complex thereof; compound 54, a pharmaceutically acceptable salt thereof, or a radionuclide complex thereof; compound 55, a pharmaceutically acceptable salt thereof, or a radionuclide complex thereof; compound 56, a pharmaceutically acceptable salt thereof, or a radionuclide complex thereof; compound 57, a pharmaceutically acceptable salt thereof, or a radionuclide complex thereof; compound 58, a pharmaceutically acceptable salt thereof, or a radionuclide complex thereof; compound 59, a pharmaceutically acceptable salt thereof, or a radionuclide complex thereof; compound 60, a pharmaceutically acceptable salt thereof, or a radionuclide complex thereof; compound 61, a pharmaceutically acceptable salt thereof, or a radionuclide complex thereof; compound 62, a pharmaceutically acceptable salt thereof, or a radionuclide complex thereof; compound 63, a pharmaceutically acceptable salt thereof, or a radionuclide complex thereof: compound 64, a pharmaceutically acceptable salt thereof, or a radionuclide complex thereof; compound 65, a pharmaceutically acceptable salt thereof, or a radionuclide complex thereof; compound 66, a pharmaceutically acceptable salt thereof, or a radionuclide complex thereof; compound 67, a pharmaceutically acceptable salt thereof, or a radionuclide complex thereof; compound 68, a pharmaceutically acceptable salt thereof, or a radionuclide complex thereof; compound 69, a pharmaceutically acceptable salt thereof, or a radionuclide complex thereof; compound 70, a pharmaceutically acceptable salt thereof, or a radionuclide complex thereof; compound 71, a pharmaceutically acceptable salt thereof, or a radionuclide complex thereof: compound 72, a pharmaceutically acceptable salt thereof, or a radionuclide complex thereof; compound 73, a pharmaceutically acceptable salt thereof, or a radionuclide complex thereof; compound 74, a pharmaceutically acceptable salt thereof, or a radionuclide complex thereof; compound 75, a pharmaceutically acceptable salt thereof, or a radionuclide complex thereof; compound 76, a pharmaceutically acceptable salt thereof, or a radionuclide complex thereof; compound 77, a pharmaceutically acceptable salt thereof, or a radionuclide complex thereof, compound 78, a pharmaceutically acceptable salt thereof, or a radionuclide complex thereof; compound 79, a pharmaceutically acceptable salt thereof, or a radionuclide complex thereof; compound 80, a pharmaceutically acceptable salt thereof, or a radionuclide complex thereof; compound 81, a pharmaceutically acceptable salt thereof, or a radionuclide complex thereof, compound 82, a pharmaceutically acceptable salt thereof, or a radionuclide complex thereof; compound 83, a pharmaceutically acceptable salt thereof, or a radionuclide complex thereof; compound 84, a pharmaceutically acceptable salt thereof, or a radionuclide complex thereof; compound 85, a pharmaceutically acceptable salt thereof, or a radionuclide complex thereof; compound 86, a pharmaceutically acceptable salt thereof, or a radionuclide complex thereof; compound 87, a pharmaceutically acceptable salt thereof, or a radionuclide complex thereof; compound 88, a pharmaceutically acceptable salt thereof, or a radionuclide complex thereof; compound 89, a pharmaceutically acceptable salt thereof, or a radionuclide complex thereof; compound 90, a pharmaceutically acceptable salt thereof, or a radionuclide complex thereof; compound 91, a pharmaceutically acceptable salt thereof, or a radionuclide complex thereof; compound 92, a pharmaceutically acceptable salt thereof, or a radionuclide complex thereof; compound 93, a pharmaceutically acceptable salt thereof, or a radionuclide complex thereof; compound 93, a pharmaceutically acceptable salt thereof, or a radionuclide complex thereof; compound 95, a pharmaceutically acceptable salt thereof, or a radionuclide complex thereof: compound 96, a pharmaceutically acceptable salt thereof, or a radionuclide complex thereof; compound 96, a pharmaceutically acceptable salt thereof, or a radionuclide complex thereof; compound 98, a pharmaceutically acceptable salt thereof, or a radionuclide complex thereof; compound 99, a pharmaceutically acceptable salt thereof, or a radionuclide complex thereof; compound 100, a pharmaceutically acceptable salt thereof, or a radionuclide complex thereof; compound 101, a pharmaceutically acceptable salt thereof, or a radionuclide complex thereof: compound 102, a pharmaceutically acceptable salt thereof, or a radionuclide complex thereof; compound 103, a pharmaceutically acceptable salt thereof, or a radionuclide complex thereof; compound 104, a pharmaceutically acceptable salt thereof, or a radionuclide complex thereof; compound 105, a pharmaceutically acceptable salt thereof, or a radionuclide complex thereof; compound 105-In, a pharmaceutically acceptable salt thereof, or a radionuclide complex thereof; compound 106, a pharmaceutically acceptable salt thereof, or a radionuclide complex thereof; compound 107, a pharmaceutically acceptable salt thereof, or a radionuclide complex thereof, compound 108, a pharmaceutically acceptable salt thereof, or a radionuclide complex thereof; compound 109, a pharmaceutically acceptable salt thereof, or a radionuclide complex thereof; compound 110, a pharmaceutically acceptable salt thereof, or a radionuclide complex thereof; compound 112, a pharmaceutically acceptable salt thereof, or a radionuclide complex thereof, compound 113, a pharmaceutically acceptable salt thereof, or a radionuclide complex thereof; compound 114, a pharmaceutically acceptable salt thereof, or a radionuclide complex thereof; compound 115, a pharmaceutically acceptable salt thereof, or a radionuclide complex thereof; compound 116, a pharmaceutically acceptable salt thereof, or a radionuclide complex thereof; compound 117, a pharmaceutically acceptable salt thereof, or a radionuclide complex thereof; compound 118, a pharmaceutically acceptable salt thereof, or a radionuclide complex thereof; compound 118-Lu, a pharmaceutically acceptable salt thereof, or a radionuclide complex thereof; compound 119, a pharmaceutically acceptable salt thereof, or a radionuclide complex thereof; compound 120, a pharmaceutically acceptable salt thereof, or a radionuclide complex thereof; compound 121, a pharmaceutically acceptable salt thereof, or a radionuclide complex thereof; compound 122, a pharmaceutically acceptable salt thereof, or a radionuclide complex thereof; compound 123, a pharmaceutically acceptable salt thereof, or a radionuclide complex thereof; compound 130, a pharmaceutically acceptable salt thereof, or a radionuclide complex thereof; compound 131, a pharmaceutically acceptable salt thereof, or a radionuclide complex thereof; compound 132, a pharmaceutically acceptable salt thereof, or a radionuclide complex thereof; compound 133, a pharmaceutically acceptable salt thereof, or a radionuclide complex thereof; compound 134, a pharmaceutically acceptable salt thereof, or a radionuclide complex thereof; compound 135, a pharmaceutically acceptable salt thereof, or a radionuclide complex thereof; compound 136, a pharmaceutically acceptable salt thereof, or a radionuclide complex thereof; compound 13⁷, a pharmaceutically acceptable salt thereof, or a radionuclide complex thereof; compound 138, a pharmaceutically acceptable salt thereof, or a radionuclide complex thereof; compound 139, a pharmaceutically acceptable salt thereof, or a radionuclide complex thereof; compound 140, a pharmaceutically acceptable salt thereof, or a radionuclide complex thereof; compound 141, a pharmaceutically acceptable salt thereof, or a radionuclide complex thereof; compound 142, a pharmaceutically acceptable salt thereof, or a radionuclide complex thereof; compound 143, a pharmaceutically acceptable salt thereof, or a radionuclide complex thereof; compound 144, a pharmaceutically acceptable salt thereof, or a radionuclide complex thereof; compound 145, a pharmaceutically acceptable salt thereof, or a radionuclide complex thereof; compound 146, a pharmaceutically acceptable salt thereof, or a radionuclide complex thereof; compound 147, a pharmaceutically acceptable salt thereof, or a radionuclide complex thereof; compound 148, a pharmaceutically acceptable salt thereof, or a radionuclide complex thereof; compound 149, a pharmaceutically acceptable salt thereof, or a radionuclide complex thereof; compound 150, a pharmaceutically acceptable salt thereof, or a radionuclide complex thereof; compound 151, a pharmaceutically acceptable salt thereof, or a radionuclide complex thereof; compound 152, a pharmaceutically acceptable salt thereof, or a radionuclide complex thereof; compound 153, a pharmaceutically acceptable salt thereof, or a radionuclide complex thereof; compound 154, a pharmaceutically acceptable salt thereof, or a radionuclide complex thereof; compound 155, a pharmaceutically acceptable salt thereof, or a radionuclide complex thereof; compound 156, a pharmaceutically acceptable salt thereof, or a radionuclide complex thereof; compound 157, a pharmaceutically acceptable salt thereof, or a radionuclide complex thereof; compound 158, a pharmaceutically acceptable salt thereof, or a radionuclide complex thereof; compound 159, a pharmaceutically acceptable salt thereof, or a radionuclide complex thereof; compound 160, a pharmaceutically acceptable salt thereof, or a radionuclide complex thereof; compound 161, a pharmaceutically acceptable salt thereof, or a radionuclide complex thereof; compound 162, a pharmaceutically acceptable salt thereof, or a radionuclide complex thereof; compound 163, a pharmaceutically acceptable salt thereof, or a radionuclide complex thereof; compound 164, a pharmaceutically acceptable salt thereof, or a radionuclide complex thereof; compound 165, a pharmaceutically acceptable salt thereof, or a radionuclide complex thereof; compound 166, a pharmaceutically acceptable salt thereof, or a radionuclide complex thereof; compound 167, a pharmaceutically acceptable salt thereof, or a radionuclide complex thereof; compound 168, a pharmaceutically acceptable salt thereof, or a radionuclide complex thereof; compound 169, a pharmaceutically acceptable salt thereof, or a radionuclide complex thereof; compound 170, a pharmaceutically acceptable salt thereof, or a radionuclide complex thereof; compound 171, a pharmaceutically acceptable salt thereof, or a radionuclide complex thereof; compound 174, a pharmaceutically acceptable salt thereof, or a radionuclide complex thereof; compound 175, a pharmaceutically acceptable salt thereof, or a radionuclide complex thereof; compound 176, a pharmaceutically acceptable salt thereof, or a radionuclide complex thereof; compound 177, a pharmaceutically acceptable salt thereof, or a radionuclide complex thereof; compound 178, a pharmaceutically acceptable salt thereof, or a radionuclide complex thereof; compound 179, a pharmaceutically acceptable salt thereof, or a radionuclide complex thereof; compound 180, a pharmaceutically acceptable salt thereof, or a radionuclide complex thereof; compound 181, a pharmaceutically acceptable salt thereof, or a radionuclide complex thereof; compound 182, a pharmaceutically acceptable salt thereof, or a radionuclide complex thereof; compound 183, a pharmaceutically acceptable salt thereof, or a radionuclide complex thereof; compound 184, a pharmaceutically acceptable salt thereof, or a radionuclide complex thereof; compound 185, a pharmaceutically acceptable salt thereof, or a radionuclide complex thereof; compound 186, a pharmaceutically acceptable salt thereof, or a radionuclide complex thereof; compound 187, a pharmaceutically acceptable salt thereof, or a radionuclide complex thereof; compound 188, a pharmaceutically acceptable salt thereof, or a radionuclide complex thereof; compound 189, a pharmaceutically acceptable salt thereof, or a radionuclide complex thereof; compound 180, a pharmaceutically acceptable salt thereof, or a radionuclide complex thereof; compound 181, a pharmaceutically acceptable salt thereof, or a radionuclide complex thereof; compound 182, a pharmaceutically acceptable salt thereof, or a radionuclide complex thereof; compound 183, a pharmaceutically acceptable salt thereof, or a radionuclide complex thereof; compound 184, a pharmaceutically acceptable salt thereof, or a radionuclide complex thereof; compound 185, a pharmaceutically acceptable salt thereof, or a radionuclide complex thereof; compound 186, a pharmaceutically acceptable salt thereof, or a radionuclide complex thereof; compound 187, a pharmaceutically acceptable salt thereof, or a radionuclide complex thereof; compound 188, a pharmaceutically acceptable salt thereof, or a radionuclide complex thereof; compound 189, a pharmaceutically acceptable salt thereof, or a radionuclide complex thereof; compound 190, a pharmaceutically acceptable salt thereof, or a radionuclide complex thereof; compound 191, a pharmaceutically acceptable salt thereof, or a radionuclide complex thereof; compound 192, a pharmaceutically acceptable salt thereof, or a radionuclide complex thereof; compound 193, a pharmaceutically acceptable salt thereof, or a radionuclide complex thereof; compound 193-In, a pharmaceutically acceptable salt thereof, or a radionuclide complex thereof; compound 194, a pharmaceutically acceptable salt thereof, or a radionuclide complex thereof; compound 195, a pharmaceutically acceptable salt thereof, or a radionuclide complex thereof; compound 196, a pharmaceutically acceptable salt thereof, or a radionuclide complex thereof; compound 196-In, a pharmaceutically acceptable salt thereof, or a radionuclide complex thereof; compound 197, a pharmaceutically acceptable salt thereof, or a radionuclide complex thereof; compound 198, a pharmaceutically acceptable salt thereof, or a radionuclide complex thereof; compound 199, a pharmaceutically acceptable salt thereof, or a radionuclide complex thereof; compound 201, a pharmaceutically acceptable salt thereof, or a radionuclide complex thereof; compound 201-In, a pharmaceutically acceptable salt thereof, or a radionuclide complex thereof; compound 202, a pharmaceutically acceptable salt thereof, or a radionuclide complex thereof; compound 203, a pharmaceutically acceptable salt thereof, or a radionuclide complex thereof; compound 204, a pharmaceutically acceptable salt thereof, or a radionuclide complex thereof; compound 205, a pharmaceutically acceptable salt thereof, or a radionuclide complex thereof; compound 206, a pharmaceutically acceptable salt thereof, or a radionuclide complex thereof; compound 207, a pharmaceutically acceptable salt thereof, or a radionuclide complex thereof; compound 208, a pharmaceutically acceptable salt thereof, or a radionuclide complex thereof; compound 209, a pharmaceutically acceptable salt thereof, or a radionuclide complex thereof; compound 210, a pharmaceutically acceptable salt thereof, or a radionuclide complex thereof; compound 211, a pharmaceutically acceptable salt thereof, or a radionuclide complex thereof; compound 212, a pharmaceutically acceptable salt thereof, or a radionuclide complex thereof; compound 213, a pharmaceutically acceptable salt thereof, or a radionuclide complex thereof; compound 214, a pharmaceutically acceptable salt thereof, or a radionuclide complex thereof; compound 215, a pharmaceutically acceptable salt thereof, or a radionuclide complex thereof; compound 216, a pharmaceutically acceptable salt thereof, or a radionuclide complex thereof; compound 217, a pharmaceutically acceptable salt thereof, or a radionuclide complex thereof; compound 218, a pharmaceutically acceptable salt thereof, or a radionuclide complex thereof; compound 219, a pharmaceutically acceptable salt thereof, or a radionuclide complex thereof; compound 220, a pharmaceutically acceptable salt thereof, or a radionuclide complex thereof; compound 221, a pharmaceutically acceptable salt thereof, or a radionuclide complex thereof; compound 222, a pharmaceutically acceptable salt thereof, or a radionuclide complex thereof; compound 224, a pharmaceutically acceptable salt thereof, or a radionuclide complex thereof; compound 225, a pharmaceutically acceptable salt thereof, or a radionuclide complex thereof; compound 226, a pharmaceutically acceptable salt thereof, or a radionuclide complex thereof; compound 227, a pharmaceutically acceptable salt thereof, or a radionuclide complex thereof; compound 229, a pharmaceutically acceptable salt thereof, or a radionuclide complex thereof; compound 230, a pharmaceutically acceptable salt thereof, or a radionuclide complex thereof; compound 231, a pharmaceutically acceptable salt thereof, or a radionuclide complex thereof; compound 232, a pharmaceutically acceptable salt thereof, or a radionuclide complex thereof; compound 233, a pharmaceutically acceptable salt thereof, or a radionuclide complex thereof; compound 234, a pharmaceutically acceptable salt thereof, or a radionuclide complex thereof; compound 235, a pharmaceutically acceptable salt thereof, or a radionuclide complex thereof; compound 236, a pharmaceutically acceptable salt thereof, or a radionuclide complex thereof; compound 237, a pharmaceutically acceptable salt thereof, or a radionuclide complex thereof; compound 238, a pharmaceutically acceptable salt thereof, or a radionuclide complex thereof; compound 239, a pharmaceutically acceptable salt thereof, or a radionuclide complex thereof; compound 240, a pharmaceutically acceptable salt thereof, or a radionuclide complex thereof; compound 241, a pharmaceutically acceptable salt thereof, or a radionuclide complex thereof; compound 242, a pharmaceutically acceptable salt thereof, or a radionuclide complex thereof; compound 243, a pharmaceutically acceptable salt thereof, or a radionuclide complex thereof; compound 244, a pharmaceutically acceptable salt thereof, or a radionuclide complex thereof; compound 245, a pharmaceutically acceptable salt thereof, or a radionuclide complex thereof; compound 246, a pharmaceutically acceptable salt thereof, or a radionuclide complex thereof; compound 247, a pharmaceutically acceptable salt thereof, or a radionuclide complex thereof; compound 248, a pharmaceutically acceptable salt thereof, or a radionuclide complex thereof; compound 249, a pharmaceutically acceptable salt thereof, or a radionuclide complex thereof; compound 240, a pharmaceutically acceptable salt thereof, or a radionuclide complex thereof; compound 241, a pharmaceutically acceptable salt thereof, or a radionuclide complex thereof; compound 242, a pharmaceutically acceptable salt thereof, or a radionuclide complex thereof; compound 243, a pharmaceutically acceptable salt thereof, or a radionuclide complex thereof; compound 244, a pharmaceutically acceptable salt thereof, or a radionuclide complex thereof; compound 245, a pharmaceutically acceptable salt thereof, or a radionuclide complex thereof; compound 246, a pharmaceutically acceptable salt thereof, or a radionuclide complex thereof; compound 247, a pharmceutically acceptable salt thereof, or a radionuclide complex thereof; compound 248, a pharmaceutically acceptable salt thereof, or a radionuclide complex thereof; compound 249, a pharmaceutically acceptable salt thereof, or a radionuclide complex thereof; compound 250, a pharmaceutically acceptable salt thereof, or a radionuclide complex thereof; compound 251, a pharmaceutically acceptable salt thereof, or a radionuclide complex thereof; compound 252, a pharmaceutically acceptable salt thereof, or a radionuclide complex thereof; compound 253, a pharmaceutically acceptable salt thereof, or a radionuclide complex thereof; compound 254, a pharmaceutically acceptable salt thereof, or a radionuclide complex thereof; compound 255, a pharmaceutically acceptable salt thereof, or a radionuclide complex thereof; compound 256, a pharmaceutically acceptable salt thereof, or a radionuclide complex thereof; compound 257, a pharmaceutically acceptable salt thereof, or a radionuclide complex thereof; compound 258, a pharmaceutically acceptable salt thereof, or a radionuclide complex thereof; compound 259, a pharmaceutically acceptable salt thereof, or a radionuclide complex thereof; compound 260, a pharmaceutically acceptable salt thereof, or a radionuclide complex thereof; compound 261, a pharmaceutically acceptable salt thereof, or a radionuclide complex thereof; compound 262, a pharmaceutically acceptable salt thereof, or a radionuclide complex thereof; compound 264, a pharmaceutically acceptable salt thereof, or a radionuclide complex thereof; compound 266, a pharmaceutically acceptable salt thereof, or a radionuclide complex thereof; compound 267, a pharmaceutically acceptable salt thereof, or a radionuclide complex thereof; compound 268, a pharmaceutically acceptable salt thereof, or a radionuclide complex thereof; compound 269, a pharmaceutically acceptable salt thereof, or a radionuclide complex thereof, compound 269-In, a pharmaceutically acceptable salt thereof, or a radionuclide complex thereof; compound 270, a pharmaceutically acceptable salt thereof, or a radionuclide complex thereof; compound 271, a pharmaceutically acceptable salt thereof, or a radionuclide complex thereof; compound 272, a pharmaceutically acceptable salt thereof, or a radionuclide complex thereof; compound 273, a pharmaceutically acceptable salt thereof, or a radionuclide complex thereof; compound 274, a pharmaceutically acceptable salt thereof, or a radionuclide complex thereof; compound 275, a pharmaceutically acceptable salt thereof, or a radionuclide complex thereof; compound 276, a pharmaceutically acceptable salt thereof, or a radionuclide complex thereof; compound 277, a pharmaceutically acceptable salt thereof, or a radionuclide complex thereof; compound 278, a pharmaceutically acceptable salt thereof, or a radionuclide complex thereof; compound 279, a pharmaceutically acceptable salt thereof, or a radionuclide complex thereof; compound 280, a pharmaceutically acceptable salt thereof, or a radionuclide complex thereof; compound 281, a pharmaceutically acceptable salt thereof, or a radionuclide complex thereof; compound 282, a pharmaceutically acceptable salt thereof, or a radionuclide complex thereof; compound 283, a pharmaceutically acceptable salt thereof, or a radionuclide complex thereof; compound 284, a pharmaceutically acceptable salt thereof, or a radionuclide complex thereof; compound 285, a pharmaceutically acceptable salt thereof, or a radionuclide complex thereof; compound 286, a pharmaceutically acceptable salt thereof, or a radionuclide complex thereof; compound 287, a pharmaceutically acceptable salt thereof, or a radionuclide complex thereof; compound 288, a pharmaceutically acceptable salt thereof, or a radionuclide complex thereof; compound 289, a pharmaceutically acceptable salt thereof, or a radionuclide complex thereof; compound 290, a pharmaceutically acceptable salt thereof, or a radionuclide complex thereof; compound 291, a pharmaceutically acceptable salt thereof, or a radionuclide complex thereof; compound 292, a pharmaceutically acceptable salt thereof, or a radionuclide complex thereof; compound 293, a pharmaceutically acceptable salt thereof, or a radionuclide complex thereof; compound 294, a pharmaceutically acceptable salt thereof, or a radionuclide complex thereof; compound 295, a pharmaceutically acceptable salt thereof, or a radionuclide complex thereof; compound 296, a pharmaceutically acceptable salt thereof, or a radionuclide complex thereof; compound 297, a pharmaceutically acceptable salt thereof, or a radionuclide complex thereof; compound 298, a pharmaceutically acceptable salt thereof, or a radionuclide complex thereof; compound 299, a pharmaceutically acceptable salt thereof, or a radionuclide complex thereof; compound 300, a pharmaceutically acceptable salt thereof, or a radionuclide complex thereof; compound 301, a pharmaceutically acceptable salt thereof, or a radionuclide complex thereof; compound 302, a pharmaceutically acceptable salt thereof, or a radionuclide complex thereof; compound 304, a pharmaceutically acceptable salt thereof, or a radionuclide complex thereof: compound 305, a pharmaceutically acceptable salt thereof, or a radionuclide complex thereof; compound 306, a pharmaceutically acceptable salt thereof, or a radionuclide complex thereof; compound 307, a pharmaceutic ally acceptable salt thereof, or a radionuclide complex thereof; compound 308, a pharmaceutically acceptable salt thereof, or a radionuclide complex thereof; compound 309, a pharmaceutically acceptable salt thereof, or a radionuclide complex thereof; compound 310, a pharmaceutically acceptable salt thereof, or a radionuclide complex thereof; compound 311, a pharmaceutically acceptable salt thereof, or a radionuclide complex thereof; compound 312, a pharmaceutically acceptable salt thereof, or a radionuclide complex thereof; compound 313, a pharmaceutically acceptable salt thereof, or a radionuclide complex thereof; compound 314, a pharmaceutically acceptable salt thereof, or a radionuclide complex thereof; compound 315, a pharmaceutically acceptable salt thereof, or a radionuclide complex thereof; compound 316, a pharmaceutically acceptable salt thereof, or a radionuclide complex thereof; compound 317, a pharmaceutically acceptable salt thereof, or a radionuclide complex thereof; compound 318, a pharmaceutically acceptable salt thereof, or a radionuclide complex thereof; compound 319, a pharmaceutically acceptable salt thereof, or a radionuclide complex thereof; compound 320, a pharmaceutically acceptable salt thereof, or a radionuclide complex thereof; compound 321, a pharmaceutically acceptable salt thereof, or a radionuclide complex thereof; compound 322, a pharmaceutically acceptable salt thereof, or a radionuclide complex thereof; compound 323, a pharmaceutically acceptable salt thereof, or a radionuclide complex thereof; compound 324, a pharmaceutically acceptable salt thereof, or a radionuclide complex thereof; compound 325, a pharmaceutically acceptable salt thereof, or a radionuclide complex thereof; compound 326, a pharmaceutically acceptable salt thereof, or a radionuclide complex thereof; compound 327, a pharmaceutically acceptable salt thereof, or a radionuclide complex thereof; compound 328, a pharmaceutically acceptable salt thereof, or a radionuclide complex thereof; compound 329, a pharmaceutically acceptable salt thereof, or a radionuclide complex thereof; compound 330, a pharmaceutically acceptable salt thereof, or a radionuclide complex thereof; compound 331, a pharmaceutically acceptable salt thereof, or a radionuclide complex thereof; compound 332, a pharmaceutically acceptable salt thereof, or a radionuclide complex thereof; compound 333, a pharmaceutically acceptable salt thereof, or a radionuclide complex thereof; compound 334, a pharmaceutically acceptable salt thereof, or a radionuclide complex thereof; compound 335, a pharmaceutically acceptable salt thereof, or a radionuclide complex thereof; compound 336, a pharmaceutically acceptable salt thereof, or a radionuclide complex thereof; compound 337, a pharmaceutically acceptable salt thereof, or a radionuclide complex thereof; compound 338, a pharmaceutically acceptable salt thereof, or a radionuclide complex thereof; compound 339, a pharmaceutically acceptable salt thereof, or a radionuclide complex thereof; compound 340, a pharmaceutically acceptable salt thereof, or a radionuclide complex thereof; compound 341, a pharmaceutically acceptable salt thereof, or a radionuclide complex thereof; compound 342, a pharmaceutically acceptable salt thereof, or a radionuclide complex thereof; compound 343, a pharmaceutically acceptable salt thereof, or a radionuclide complex thereof; compound 344, a pharmaceutically acceptable salt thereof, or a radionuclide complex thereof; compound 345, a pharmaceutically acceptable salt thereof, or a radionuclide complex thereof; compound 346, a pharmaceutically acceptable salt thereof, or a radionuclide complex thereof; compound 347, a pharmaceutically acceptable salt thereof, or a radionuclide complex thereof; compound 348, a pharmaceutically acceptable salt thereof, or a radionuclide complex thereof; compound 349, a pharmaceutically acceptable salt thereof, or a radionuclide complex thereof; compound 350, a pharmaceutically acceptable salt thereof, or a radionuclide complex thereof; compound 351, a pharmaceutically acceptable salt thereof, or a radionuclide complex thereof; compound 352, a pharmaceutically acceptable salt thereof, or a radionuclide complex thereof; compound 353, a pharmaceutically acceptable salt thereof, or a radionuclide complex thereof; compound 354, a pharmaceutically acceptable salt thereof, or a radionuclide complex thereof; compound 355, a pharmaceutically acceptable salt thereof, or a radionuclide complex thereof; compound 356, a pharmaceutically acceptable salt thereof, or a radionuclide complex thereof; compound 357, a pharmaceutically acceptable salt thereof, or a radionuclide complex thereof; compound 358, a pharmaceutically acceptable salt thereof, or a radionuclide complex thereof; compound 359, a pharmaceutically acceptable salt thereof, or a radionuclide complex thereof; compound 360, a pharmaceutically acceptable salt thereof, or a radionuclide complex thereof; compound 361, a pharmaceutically acceptable salt thereof, or a radionuclide complex thereof; compound 362, a pharmaceutically acceptable salt thereof, or a radionuclide complex thereof; compound 363, a pharmaceutically acceptable salt thereof, or a radionuclide complex thereof; compound 364, a pharmaceutically acceptable salt thereof, or a radionuclide complex thereof; compound 365, a pharmaceutically acceptable salt thereof, or a radionuclide complex thereof; compound 366, a pharmaceutically acceptable salt thereof, or a radionuclide complex thereof; compound 367, a pharmaceutically acceptable salt thereof, or a radionuclide complex thereof; compound 368, a pharmaceutically acceptable salt thereof, or a radionuclide complex thereof; compound 369, a pharmaceutically acceptable salt thereof, or a radionuclide complex thereof; compound 370, a pharmaceutically acceptable salt thereof, or a radionuclide complex thereof; compound 371, a pharmaceutically acceptable salt thereof, or a radionuclide complex thereof; compound 372, a pharmaceutically acceptable salt thereof, or a radionuclide complex thereof; compound 373, a pharmaceutically acceptable salt thereof, or a radionuclide complex thereof; compound 374, a pharmaceutically acceptable salt thereof, or a radionuclide complex thereof; compound 375, a pharmaceutically acceptable salt thereof, or a radionuclide complex thereof; compound 376, a pharmaceutically acceptable salt thereof, or a radionuclide complex thereof; compound 378, a pharmaceutically acceptable salt thereof, or a radionuclide complex thereof; compound 379, a pharmaceutically acceptable salt thereof, or a radionuclide complex thereof; compound 380, a pharmaceutically acceptable salt thereof, or a radionuclide complex thereof; compound 381, a pharmaceutically acceptable salt thereof, or a radionuclide complex thereof; compound 382, a pharmaceutically acceptable salt thereof, or a radionuclide complex thereof; compound 383, a pharmaceutically acceptable salt thereof, or a radionuclide complex thereof; compound 384, a pharmaceutically acceptable salt thereof, or a radionuclide complex thereof; compound 385, a pharmaceutically acceptable salt thereof, or a radionuclide complex thereof; compound 386, a pharmaceutically acceptable salt thereof, or a radionuclide complex thereof; compound 387, a pharmaceutically acceptable salt thereof, or a radionuclide complex thereof; compound 388, a pharmaceutically acceptable salt thereof, or a radionuclide complex thereof, or compound 389, a pharmaceutically acceptable salt thereof, or a radionuclide complex thereof.

Any combination of the groups described above for the various variables is contemplated herein. Throughout the specification, groups and substituents thereof are chosen by one skilled in the field to provide stable moieties and compounds.

Representative Kisspeptin receptor (KISS1R ligands

In one aspect, the Kisspeptin ligand described herein has the structure of Formula (lI), or a pharmaceutically acceptable salt thereof. In some embodiments, described herein is a compound of Formula (II), or a pharmaceutically acceptable salt thereof:

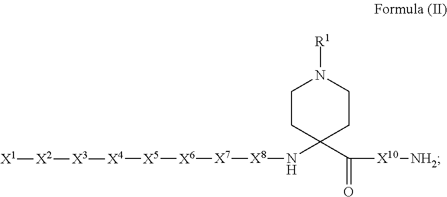

wherein:
R¹ is H,

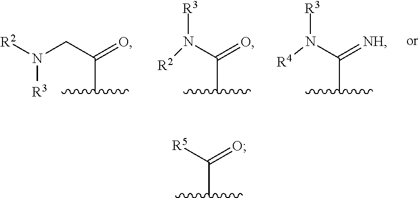

R² is C₁-C₈ alkyl, substituted or unsubstituted heteroalkyl, —(CHR⁶)ₙ-heterocycloalkyl, —(CHR⁶)ₙ-aryl, —(CHR⁶)ₙ-heteroaryl, —C(═)—(CHR⁶)ₙ-aryl, or —C(═O)NH—(CHR⁶)ₙ-aryl; wherein C₁-C₆ alkyl is optionally substituted with R⁷, and wherein the heterocycloalkyl, aryl, or heteroaryl are each independently optionally substituted with R⁷, R⁸, R⁹, R¹⁰, and R¹¹;
R³ is H or C₁-C₄ alkyl;
R⁴ is H, C₁-C₄ alkyl, or R²;
R⁵ is substituted or unsubstituted alkyl, substituted or unsubstituted heteroalkyl, or —(CHR⁶)ₙ-aryl; wherein aryl is optionally substituted with R⁷, R⁸, R⁹, R¹⁰, and R¹¹;
each R⁶ is independently H, F, —CH₃, ˜NH₂, or —OH;
R⁷, R⁸, R⁹, R¹⁰, and R¹¹ are each independently selected from H, F, Cl, Br, I, —OH, —O—C₁-C₄ alkyl, —NH₂, —NHC₁-C₄ alkyl, —N(C₁-C₄ alkyl)₂, —CN, —CO₂H, —CO₂C₁-C₄ alkyl, —C₁-C₆ alkyl, —C₁-C₆ fluroroalkyl or —C₃-C₆ cycloalkyl;
n is 0, 1, 2, 3, 4, 5, or 6;
X¹ is absent, tyrosine (Tyr), glycine (Gly), sarcosine (Sar), alanine (Ala), aspartic acid (Asp), lysine (Lys), phenylalanine (Phe), 3-(3-pyridyl)alanine (3-Pal) threonine (Thr), methionine (Met), 4-iodophenylalanine (Phe(4-1)), N6-(4-(p-tolyl)butanoyl)-lysine, N6-(4-(4-iodophenyl)butanoyl)-lysine, or γ-glutamic acid (γ-Glu));
X² is absent, asparagine (Asn), glutamine (Gln), aspartic acid (Asp), glutamic acid (Glu), serine (Ser), histidine (His), alanine (Ala), sarcosine (Sar), tyrosine (Tyr), proline (Pro), hydroxyproline (Hyp), azetidine-2-carboxylic acid (Aze), 2,3,4,5-tetrahydroisoquinoline-3-carboxylic acid (Tic), phenylalanine (Phe), 3-(2-pyridyl) alanine (2-Pal), 3-(3-pyridyl)alanine (3-Pal), or 3-(4-pyridyl)alanine (4-Pal);
X³ is absent, tryptophan (Trp), serine (Ser), leucine (Leu), isoleucine (Ile), phenylalanine (Phe), 4-iodophenylalanine (Phe(4-I)), 3-(2-pyridyl)alanine (2-Pal), 3-(3-pyridyl)alanine (3-Pal), 3-(4-pyridyl)alanine 4-Pal), 2-amino-3-(naphthalen-2-yl)propanoic acid (1-2-NAL-O)l), lysine (Lys), asparagine (Asn), glutamine (Gln), aspartic acid (Asp), glutamic acid (Glu), arginine (Arg), methyl arginine (Arg(Me)), norarginine (AGBA), methyl norarginine (AGBA(Me)), homoarginine (HArg), methyl homoarginine (HArg(Me)), citrulline (Cit), methyl citrulline (Cit(Me)), canavanine, methyl-canavanine, glycine (Gly), alanine (Ala), sarcosine (Sar), tyrosine (Tyr), cyclohexylalanine(Cha), 3-(1-naphthyl)alanine(α-Nal), 3-(2-naphthyl)alanine (β-Nal), or threonine (Thr), proline (Pro), hydroxyproline (Hyp), or tetrahydroisoquinoline-3-carboxylic acid (Tic); O-phospho-serine (SOP), 2-amino-4-(2H-tetrazol-5-yl)butanoic acid, β-glutamic acid, 8-aminoquinoline-3-carboxylic acid, biphenylalanine (Dip), 4-benzoylphenylalanine (Bpa), or 3-(9-anthryl)-alanine (H-Ala(9-Anth)-OH or AAP);
X⁴ is absent, asparagine (Asn), glutamine (Gln), aspartic acid (Asp), glutamic acid (Glu), tryptophan (Tip), glycine (Gly), tyrosine (Tyr), alanine (Ala), or sarcosine (Sar);
X⁵ is absent, serine (Ser), threonine (Thr), lysine (Lys), asparagine (Asn), glutamine (Gln), aspartic acid (Asp), glutamic acid (Glu), glycine (Gly), alanine (Ala), sarcosine (Sar), or arginine (Arg);
X⁶ is absent, phenylalanine (Phe), alpha-methylphenylalanine (α-Me-Phe), N-methylphenylalanine (N-Me-Phe), 2-fluorophenylalanine (2-F-Phe), 3-fluorophenylalanine (3-F-Phe), 4-fluorophenylalanine (4-F-Phe), 4-iodophenylalanine (Phe(4-I)), 2-amino-2-indancarboxylic acid (Aic), biphenylalanine (Bip), (β-(2-thienyl)-Ala), tryptophan (Trp), 2-aminotetralin-2-carboxylic acid (Atc); 3-(2-thienyl)-alanine, 3-(4-pyridyl)alanine (4-Pal), cyclohexylalanine (Cha), or tyrosine (Tyr);
X⁷ is absent, glycine (Gly), aza-glycine (aza-Gly), alanine (Ala), N-methylglycine (Sar), or 1-aminocyclopropane-1-carboxylic acid (ACC);
X⁸ is leucine (Leu), norvaline (Nva), valine (Val), isoleucine (Ile), homoalanine (HAla), tryptophan (Tip), phenylalanine (Phe), or phenylglycine (Phg);
or —X⁷—X⁸— is

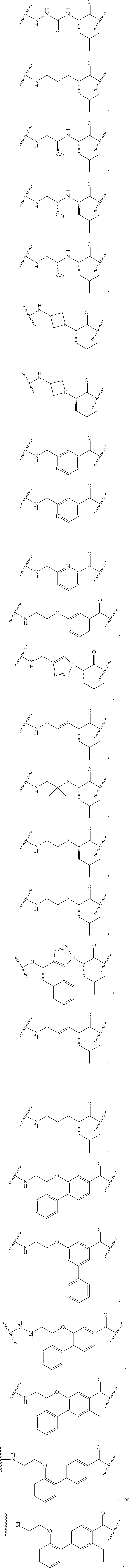

or —X⁶—X⁷—X⁸ or

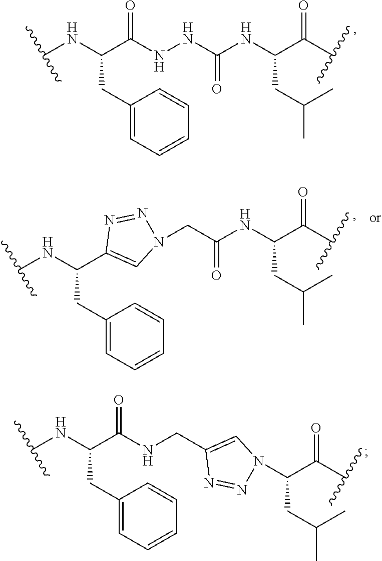

X¹⁰ is tryptophan (Trp), 1-methyltryptophan (1MT), tyrosine (Tyr), phenylalanine (Phe), 4-cyano phenylalanine (Phe(4-CN)), 3-(4-pyridyl)alanine (4-Pal), leucine (Leu), phenylglycine (Phg), cyclohexylalanine (Cha), 3(1-naphthyl)alanine (α-Nal), 3-(2-naphthyl)-alanine (β-Nal), histidine (His), or 3-nitro-tyrosine (Tyr(3-NO₂));
wherein the N-terminal amino acid or the compound of Formula (II) is optionally substituted with —C(═O)—C₁-C₂₀ alkyl, —C(═O)—(CH₂CH₂O)_{y}—CH₂CH₂—R¹⁵, —C₁-C₂₀ alkyl, N-hexadecanoyl-Glu, —C₄-C₂₀ polyethylene glycol, a saccharide, —R¹⁶, —C(═O)—(CH₂CH₂O)ₓ—CH₃, —C(═O)—(CH₂CH₂O)ₓ—, —C(═O)—CH₂CH₂CH(COOH)—R¹⁵, —C(═O)—(CH₂)₂R¹⁹, or —C(═O)CH₂NHCH₂R¹⁹;
R¹⁵ is selected from —OR¹⁶, —N(R¹⁶)₂, —C(═O)OR¹⁶, or —C(═O)N(R¹⁶)₂;
each R¹⁶ is independently H, —C₁-C₆ alkyl, —C(═O)—(CH₂)ᵥR¹⁹, —C(═O)CH₂NHCH₂R¹⁹, or a saccharide or derivative thereof;
R¹⁹ is 4-iodophenylene, 4-methylphenylene, or 3-fluoro-4-methylphenylene;
y is 0, 1, 2, 3, 4, 5, 6, 7, 8, 9, or 10;
x is an integer from 1 and 25; and
v is 1, 2, 3, or 4;
wherein any free —NH— of a peptide bond is optionally independently substituted with —CH₃ or —CH₂CH₃ and
wherein any alpha position of an amino acid is optionally independently substituted with —CH₃, or —CH₂CH₃.

In some embodiments, R¹ is

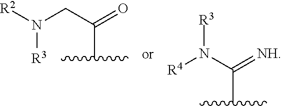

In some embodiments, R² is H and R³ is H or C₁-C₄ alkyl. In some embodiments, R² is H and R³ is H. In some embodiments, R² is H and R³ is CH₃.

In some embodiments, R³ is H and R⁴ is H or C₁-C₄ alkyl.

In some embodiments, R² is —(CHR⁶)ₙ-aryl.

In some embodiments, n is 1.

In some embodiments, R⁶ is H.

In some embodiments. R¹ is

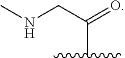 [SMILES: *C(=O)CNC]

In some embodiments, R¹ is

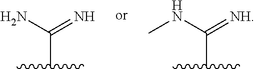

In some embodiments, R¹ is

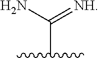 [SMILES: *C(=N)N]

In some embodiments, R¹ is

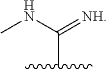 [SMILES: *C(=N)NC]

In some embodiments, R¹ is

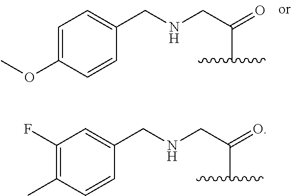 [SMILES: *C(=O)CNCc1ccc(OC)cc1 ; *C(=O)CNCc1ccc(C)c(F)c1]

In some embodiments, R¹ is

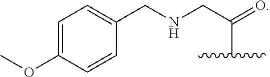 [SMILES: *C(=O)CNCc1ccc(OC)cc1]

In some embodiments, R¹ is

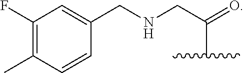 [SMILES: *C(=O)CNCc1ccc(C)c(F)c1]

In some embodiments, R¹ is

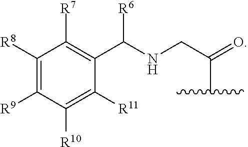

In some embodiments, R⁷, R⁸, R⁹, R¹⁰, and R¹¹ are each independently selected from H, F, Cl, Br, I, —OH, —O—C₁-C₄ alkyl, —NH₂, or —C₁-C₆ alkyl. In some embodiments, R⁷, R⁸, R⁹, R¹⁰, and R¹¹ are each independently selected from H, F, Cl, Br, I, —OH, —OCH₃, —NH₂, or —CH₃. In some embodiments, R⁷ is H. In some embodiments, R⁷ is F. In some embodiments, R⁷ is Cl. In some embodiments, R⁷ is Br. In some embodiments, R⁷ is I. In some embodiments, R⁷ is —OH. In some embodiments, R⁷ is —OCH₃. In some embodiments, R⁷ is —NH₂. In some embodiments, R⁷ is —CH₃. In some embodiments, R⁸ is H. In some embodiments, R⁸ is F. In some embodiments, R⁸ is Cl. In some embodiments, R⁸ is Br. In some embodiments, R⁸ is I. In some embodiments, R⁸ is —OH. In some embodiments, R¹ is —OCH₃. In some embodiments, R⁸ is ˜NH₂. In some embodiments, R¹⁰ is —CH₃. In some embodiments, R⁹ is H. In some embodiments, R⁹ is F. In some embodiments, R⁹ is Cl. In some embodiments, R⁹ is Br. In some embodiments, R⁹ is I. In some embodiments, R⁹ is —CH₃. In some embodiments, R⁹ is —OCH₃. In some embodiments, R⁹ is ˜NH₂. In some embodiments. R⁹ is —Cl. In some embodiments, R¹⁰ is H. In some embodiments, R¹⁰ is F. In some embodiments, R¹⁰ is Cl. In some embodiments, R¹⁰ is Br. In some embodiments, R¹⁰ is I. In some embodiments, R¹⁰ is —OH. In some embodiments, R¹⁰ is —OCH₃. In some embodiments, R¹⁰ is —NH₂. In some embodiments, R¹⁰ is —CH₃—. In some embodiments, R¹¹ is H. In some embodiments, R¹¹ is F. In some embodiments, R¹¹ is Cl. In some embodiments, R¹¹ is Br. In some embodiments, R¹¹ is I. In some embodiments, R¹¹ is —OH. In some embodiments, R¹¹ is —OCH₃. In some embodiments, R¹¹ is —NH₂. In some embodiments, R¹¹ is —CH₃. In some embodiments, R¹¹ is F and R⁹ is CH₃.

In some embodiments, X is tyrosine (Tyr

In some embodiments, X² is absent.

In some embodiments, X³ is 3-(2-naphthyl)alanine (β-Nal). In some embodiments, X³ is tryptophan (Trp).

In some embodiments, X⁴ is asparagine (Asn).

In some embodiments, X⁵ is threonine (Thr).

In some embodiments, X⁶ is phenylalanine (Phe). In some embodiments, X⁶ is cyclohexylalanine (Cha).

In some embodiments, X⁷ is azaglycine (aza-gly).

In some embodiments, X⁸ is leucine (Leu).

In some embodiments, X¹⁰ is tryptophan (Trp), 1-methyltryptophan (1MT), tyrosine (Tyr), phenylalanine(Phe), 4-cyanophenylalanine(Phe(4-CN)), 3-(4-pyridyl)alanine (4-Pal), or leucine (Leu). In some embodiments, X¹⁰ is tryptophan (Trp), tyrosine (Tyr), or phenylalanine (Phe). In some embodiments, X¹⁰ is tryptophan (Trp). In some embodiments, X¹⁰ is I-methyltryptophan (1MT). In some embodiments, X¹⁰ is tyrosine (Tyr). In some embodiments, X¹⁰ is phenylalanine (Phe), in some embodiments, X¹⁰ is 4-cyano phenylalanine (Phe(4-CN)). In some embodiments, X¹⁰ is 3-(4-pyridyl)alanine (4-Pal). In some embodiments, X¹⁰ is leucine (Leu).

In some embodiments, X is absent, tyrosine (Tyr), or 3-(3-pyridyl)alanine (3-Pal));
X² is absent, asparagine (Asn), glutamine (Gln), serine (Ser), D-histidine (His), or phenylalanine (Phe);
X³ is absent, tryptophan (Trp), isoleucine (Ile), 3-(4-pyridyl)alanine (4-Pal), lysine (Lys), aspartic acid (Asp), glutamic acid (Glu), glycine (Gly), alanine (Ala), cyclohexylalanine (Cha), (Hyp), biphenylalanine (Bip); 4-benzoylphenylalanine (Bpa), or 3-(9-anthryl)-alanine (AAP);
X⁴ is absent, asparagine (Asn), or glutamine (Gln); and
X⁵ is absent, serine (Ser), threonine (Thr), glycine (Gly), or alanine (Ala).

In some embodiments, X¹ is D-tyrosine (D-Tyr);
X² is absent;
X³ is D-tryptophan (D-Trp), biphenylalanine (Bip); 4-benzoylphenylalanine (Bpa), or 3-(9-anthryl)-alanine (AAP);
X⁴ is asparagine (Asn); and
X⁵ is serine (Ser) or threonine (Thr).

In some embodiments. X⁶ is phenylalanine (Phe), 3-fluorophenylalanine (3-F-Phe), biphenylalanine (Bip), or cyclohexylalanine (Cha); X⁷ is glycine (Gly) or aza-glycine (aza-Gly); and X⁸ is leucine (Leu) or norvaline (Nva). In some embodiments, X⁶ is phenylalanine (Phe) or cyclohexylalanine (Cha); X⁷ is aza-glycine (aza-Gly); and X⁸ is leucine (Leu).

In some embodiments, X⁸ is Leu, Nya, Ile, Hala, or Phe; and X¹⁰ is Trp, 1MT, Tyr, 4-Pal, Phe(4-CN), or Phe.

In some embodiments,

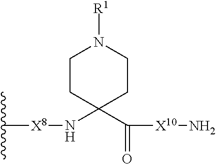

is

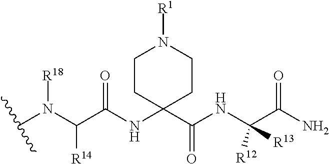

wherein,
R¹⁸ is H or —CH₃;
R¹² is

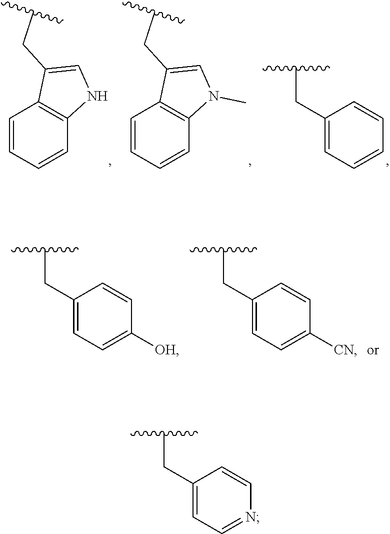

R¹³ is H or —CH₃; and
R¹⁴ is

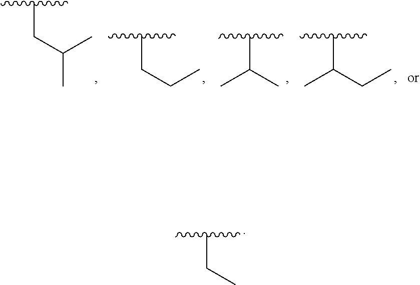

In some embodiments, the N-terminal amino acid or the compound of Formula (I I) is optionally substituted with —C(═O)—C₁-C₂₀ alkyl, —C(═O)—(CH₂CH₂O)_{y}, —CH₂CH₂—R¹⁵, —C₁-C₂₀ alkyl, N-hexadecanoyl-Glu, —C₄-C₂₀ polyethylene glycol, a saccharide, —R¹⁶, —C(═O)—(CH₂CH₂O)ₓ—CH₃, —(CH₂CH₂O)ₓ—H, —C(═O)—C₂CH₂CH(COOH)—R¹⁵, —C(═O)—(CH₂)₂R¹⁹, or —C(O)CH₂NHCH₂R¹⁹;
R¹⁵ is selected from —OR¹⁶, —N(R¹⁶)₂, —C(═O)OR¹⁶, or —C(═O)N(R⁶)₂;
each R¹⁶ is independently H, —C₁-C₆ alkyl, —C(═O)—(CH₂)_{y}R¹⁹, —C(═O)CH₂NHCH₂R¹⁹, or a saccharide or derivative thereof;
   R¹⁹ is 4-iodophenylene, 4-methylphenylene, or 3-fluoro-4-methylphenylene;
y is 0, 1, 2, 3, 4, 5, 6, 7, 8, 9, or 10;
x is an integer from 1 and 25; and
v is 1, 2, 3, or 4.

In some embodiments, the N-terminal amino acid or the compound of Formula (II) is optionally substituted with —C(═O)—C₁-C₁₂alkyl.

In some embodiments, the N-terminal amino acid or the compound of Formula (II) is optionally substituted with —C(═O)—(CH₂CH₂O)—CH₂CH₂—R¹⁵. In some embodiments, y is 2. In some embodiments, R¹⁵ is —N(R¹⁶)₂ and both R¹⁶ are H. In some embodiments, R¹⁵ is —N(R¹⁶)₂, one R¹⁶ is H and the other R¹⁶ is —C(═O)—(CHR²)ᵥR¹⁹. In some embodiments, the N-terminal amino acid or the compound of Formula (II) is optionally substituted with —R¹⁶. In some embodiments, R¹⁶ is —C(═O)—(CH₂)ᵥR¹⁹. In some embodiments, R¹⁹ is 2 or 3. In some embodiments, R¹⁹ is 4-iodophenylene or 4-methylphenylene.

In some embodiments, the N-terminal amino acid or the compound of Formula (II) is optionally substituted with

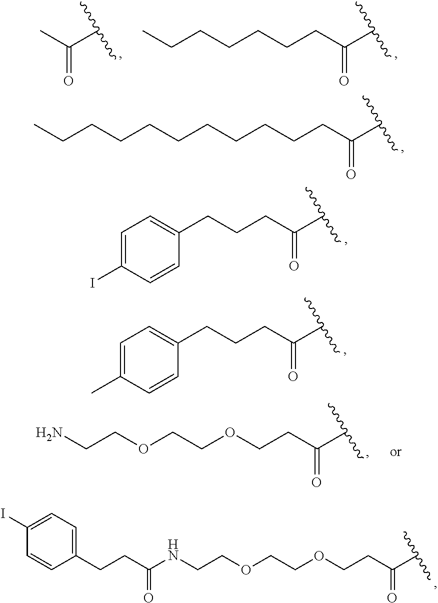

In some embodiments, the N-terminal amino acid or the compound of Formula (II) is optionally substituted with

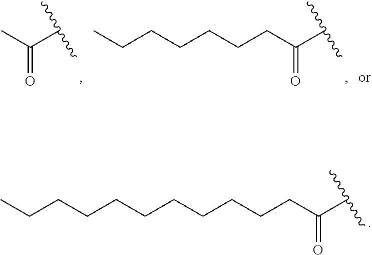

In some embodiments, the N-terminal amino acid or the compound of Formula (II) is optionally substituted with

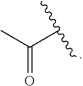 [SMILES: *C(C)=O]

In some embodiments, the N-terminal amino acid or the compound of Formula (II) is optionally substituted with

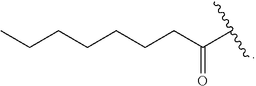 [SMILES: *C(=O)CCCCCCC]

In some embodiments, the N-terminal amino acid or the compound of Formula (II) is optionally substituted with

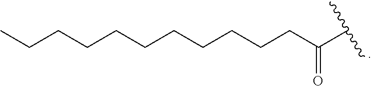 [SMILES: *C(=O)CCCCCCCCCCC]

In some embodiments, the N-terminal amino acid or the compound of Formula (II) is optionally substituted with

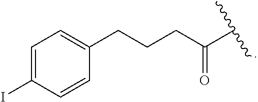 [SMILES: *C(=O)CCCc1ccc(I)cc1]

In some embodiments, the N-terminal amino acid or the compound of Formula (II) is optionally substituted with

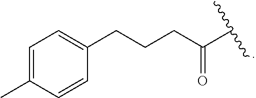 [SMILES: *C(=O)CCCc1ccc(C)cc1]

In some embodiments, the N-terminal amino acid or the compound of Formula (II) is optionally substituted with

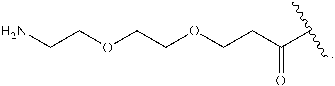 [SMILES: *C(=O)CCOCCOCCN]

In some embodiments, the N-terminal amino acid or the compound of Formula (II) is optionally substituted with

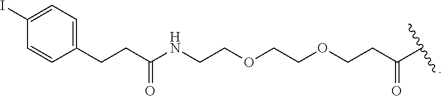 [SMILES: *C(=O)CCOCCOCCNC(=O)CCc1ccc(I)cc1]

In some embodiments, the compound of Formula (II) has the following structure, or a pharmaceutically acceptable salt thereof:

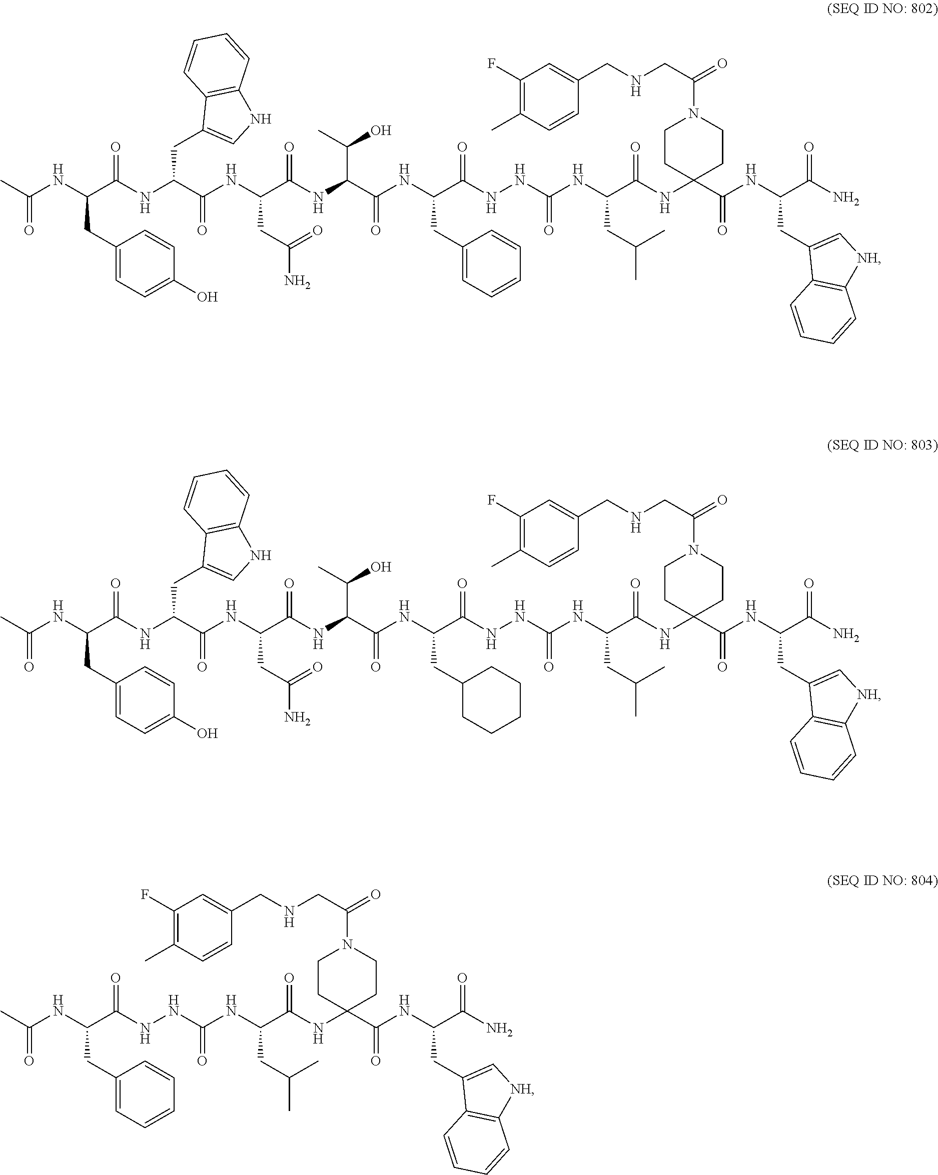

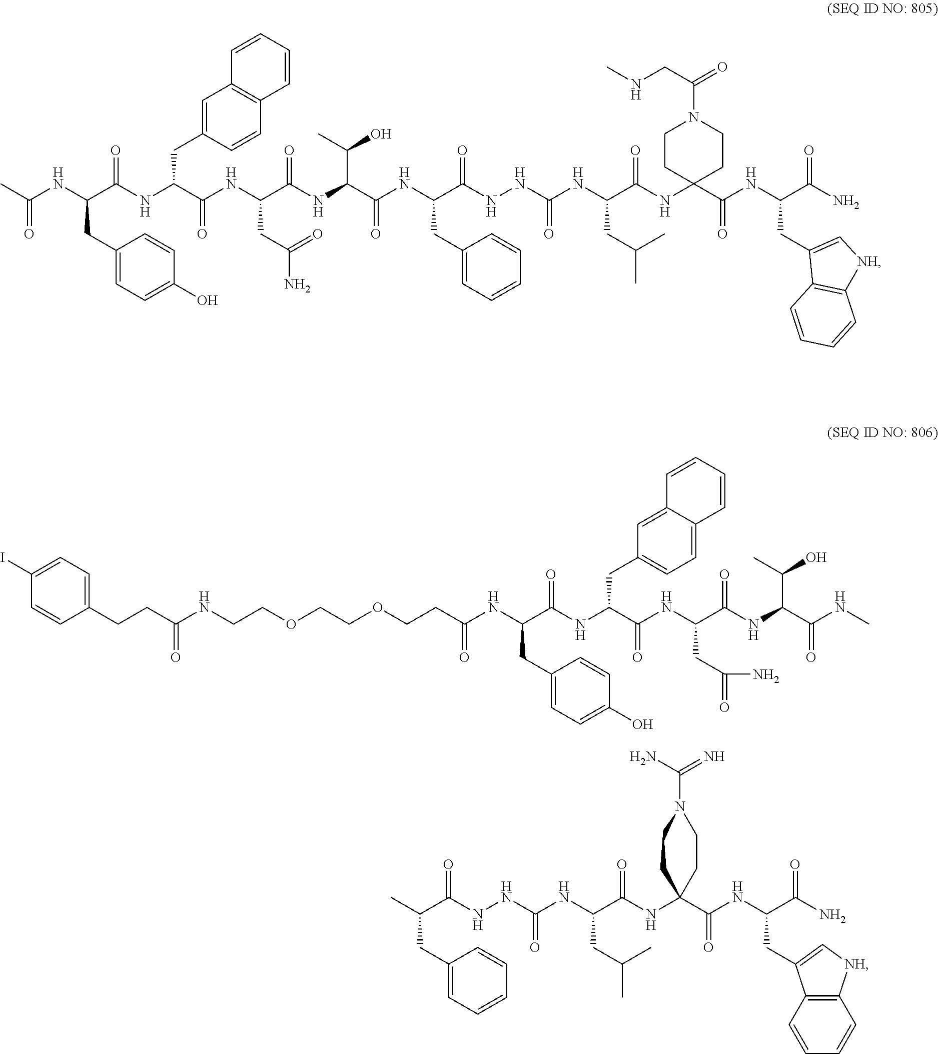

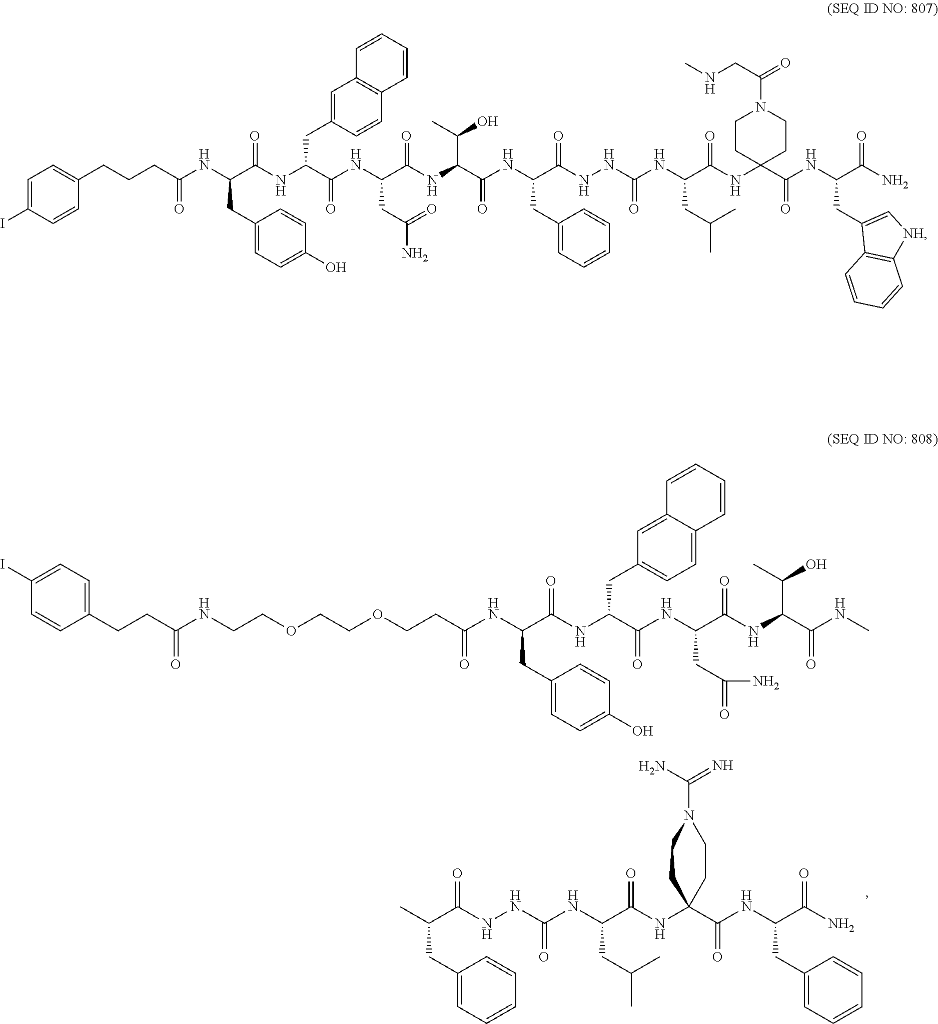

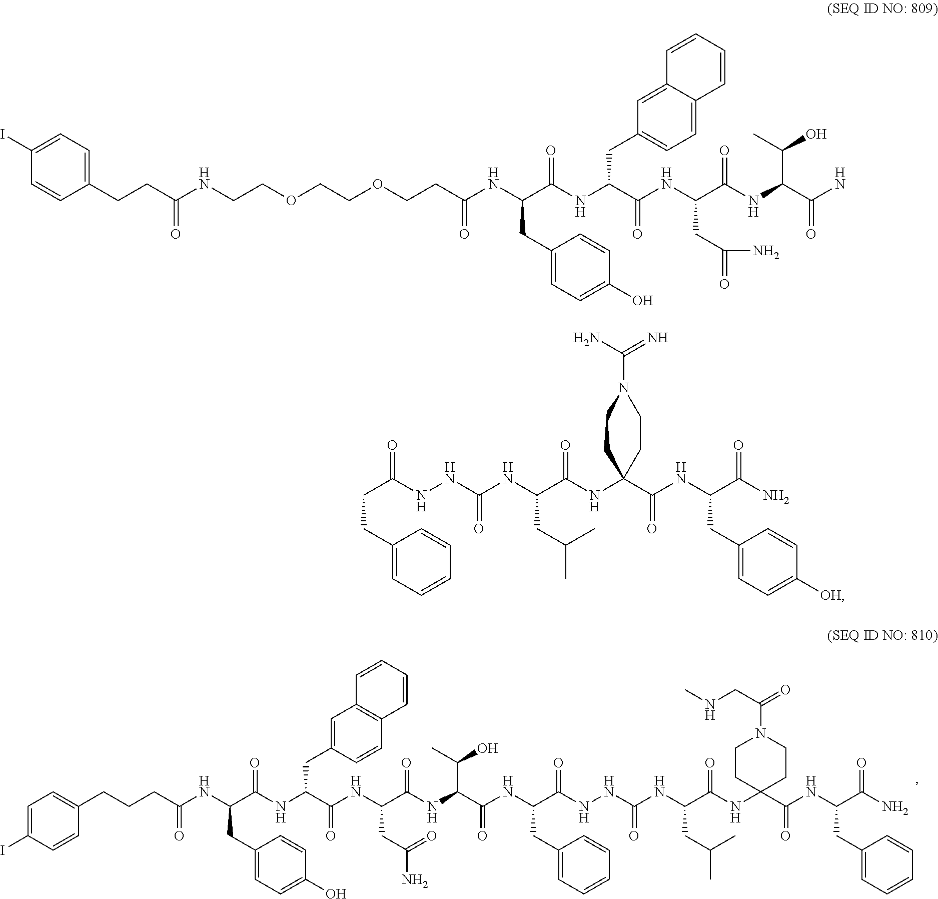

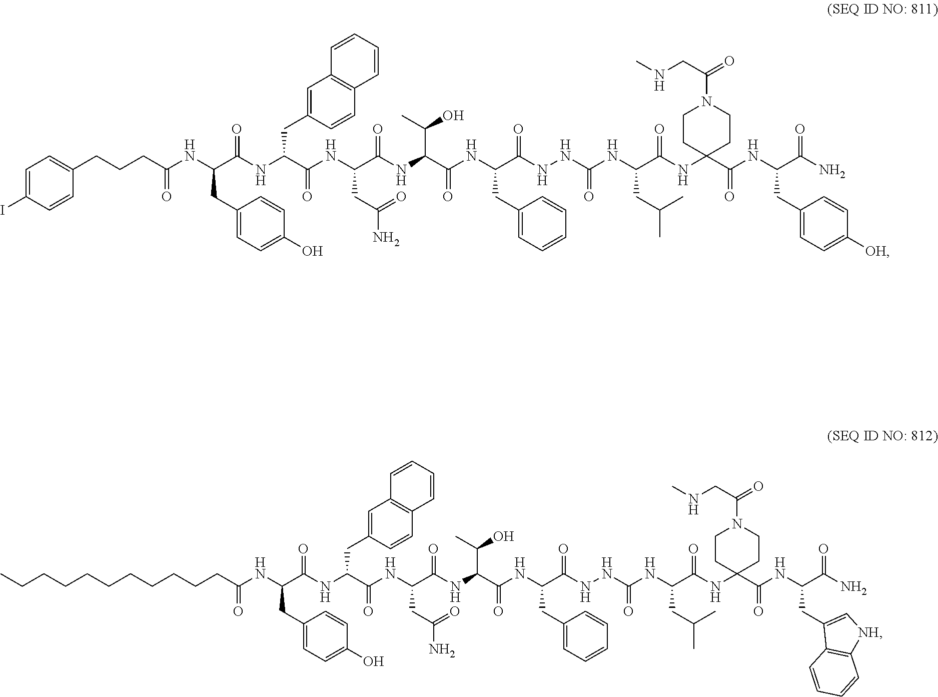 [SMILES: CNCC(=O)N1CCC(NC(=O)[C@H](CC(C)C)NC(=O)NNC(=O)[C@H](Cc2ccccc2)NC(=O)[C@@H](NC(=O)[C@H](CC(N)=O)NC(=O)[C@@H](Cc2ccc3ccccc3c2)NC(=O)[C@@H](Cc2ccc(O)cc2)NC(=O)CCCc2ccc(I)cc2)[C@@H](C)O)(C(=O)N[C@@H](Cc2ccc(O)cc2)C(N)=O)CC1 ; CCCCCCCCCCCC(=O)N[C@H](Cc1ccc(O)cc1)C(=O)N[C@H](Cc1ccc2ccccc2c1)C(=O)N[C@@H](CC(N)=O)C(=O)N[C@H](C(=O)N[C@@H](Cc1ccccc1)C(=O)NNC(=O)N[C@@H](CC(C)C)C(=O)NC1(C(=O)N[C@@H](Cc2c[nH]c3ccccc23)C(N)=O)CCN(C(=O)CNC)CC1)[C@@H](C)O]

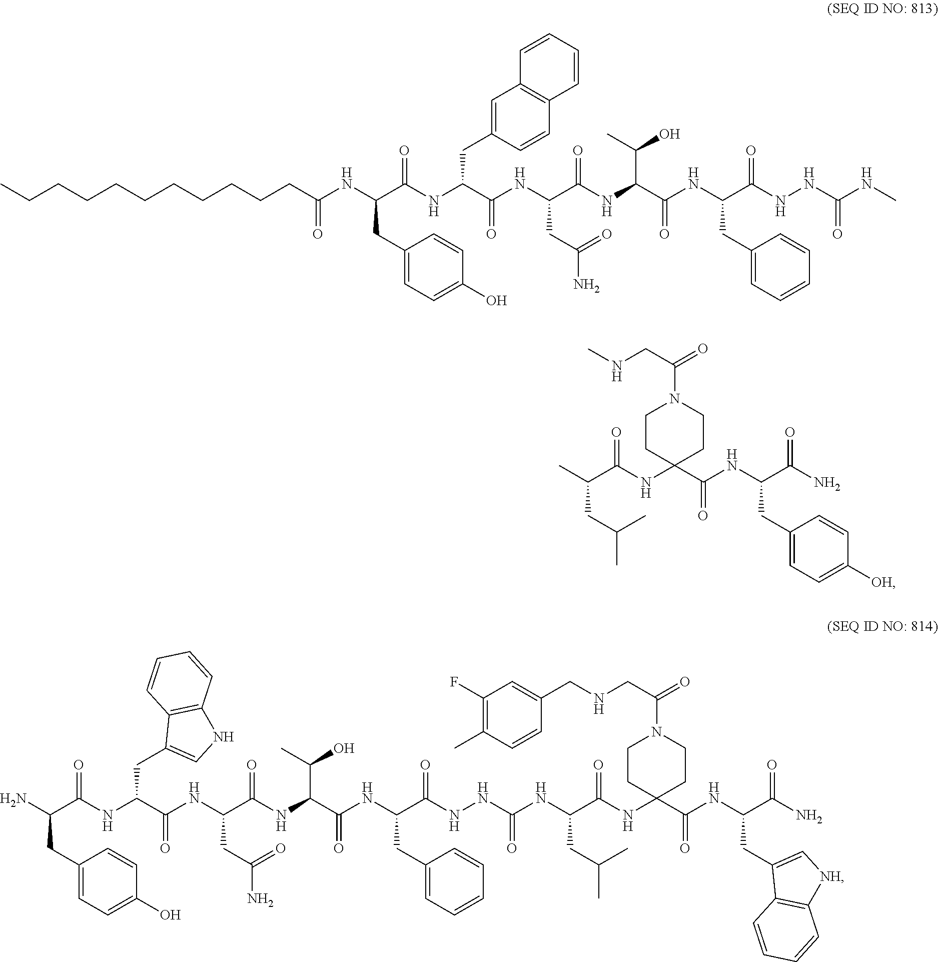

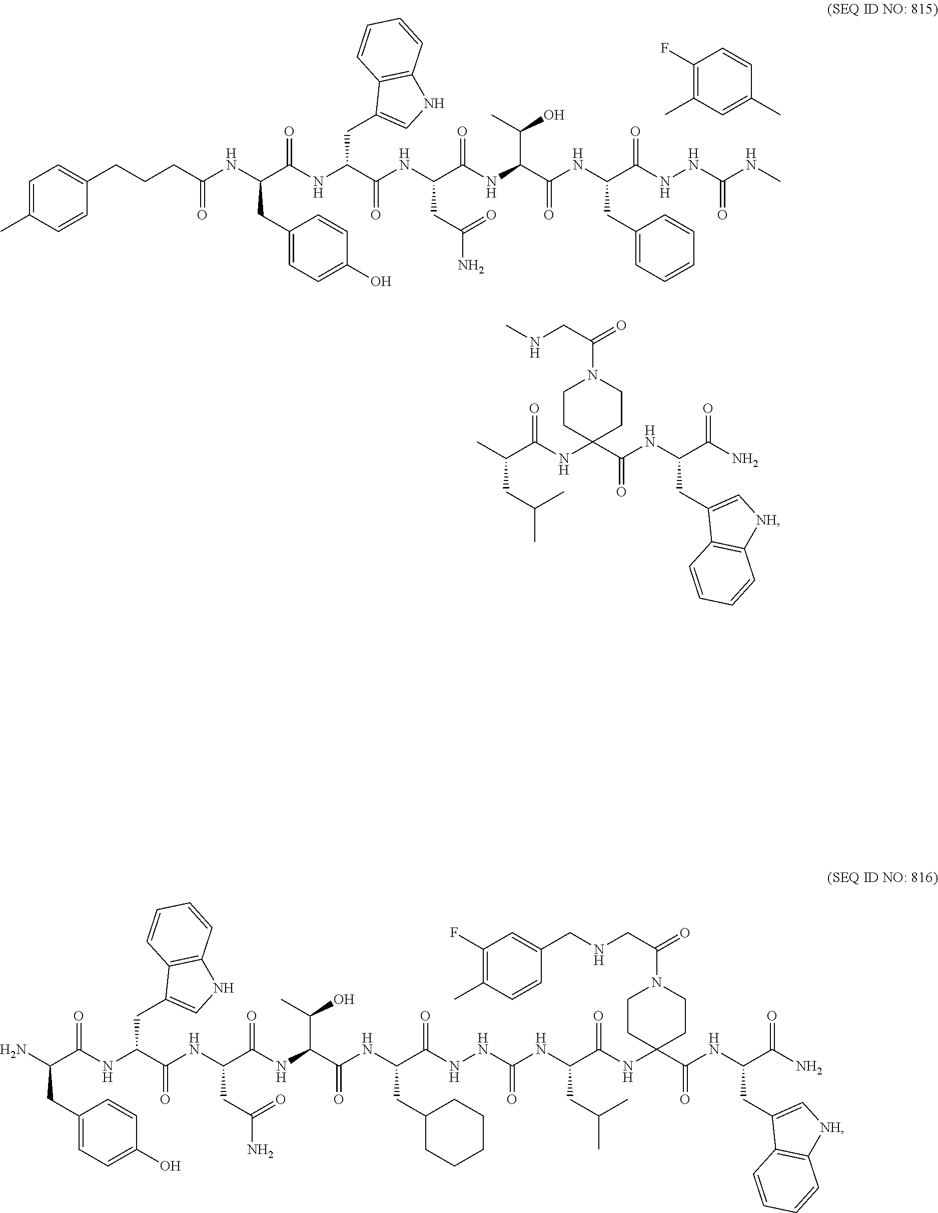

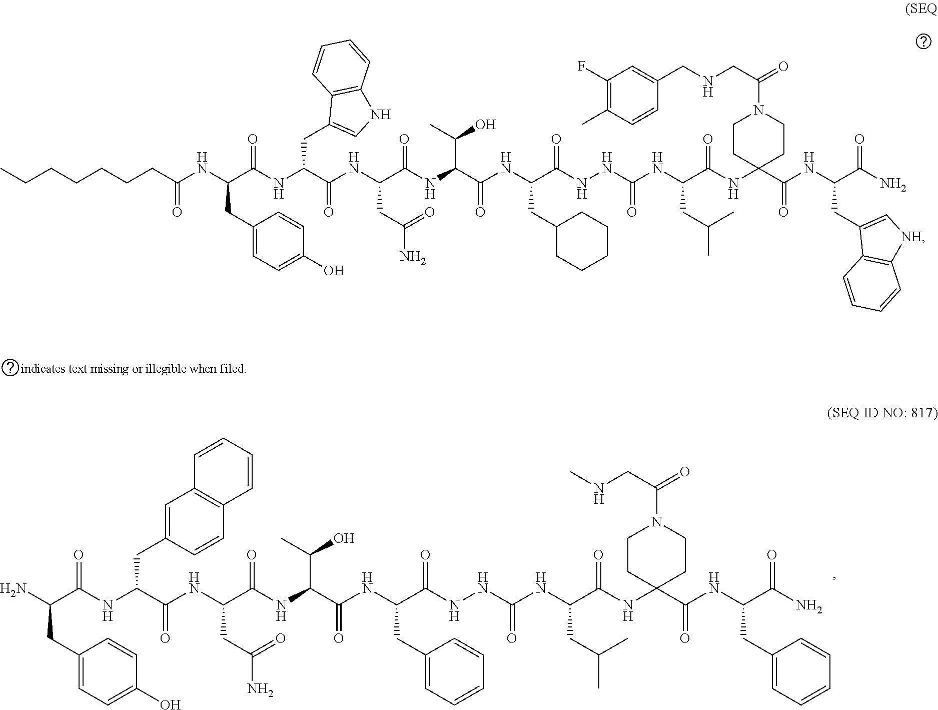 [SMILES: CCCCCCCC(=O)N[C@H](Cc1ccc(O)cc1)C(=O)N[C@H](Cc1c[nH]c2ccccc12)C(=O)N[C@@H](CC(N)=O)C(=O)N[C@H](C(=O)N[C@@H](CC1CCCCC1)C(=O)NNC(=O)N[C@@H](CC(C)C)C(=O)NC1(C(=O)N[C@@H](Cc2c[nH]c3ccccc23)C(N)=O)CCN(C(=O)CNCc2ccc(C)c(F)c2)CC1)[C@@H](C)O ; CNCC(=O)N1CCC(NC(=O)[C@H](CC(C)C)NC(=O)NNC(=O)[C@H](Cc2ccccc2)NC(=O)[C@@H](NC(=O)[C@H](CC(N)=O)NC(=O)[C@@H](Cc2ccc3ccccc3c2)NC(=O)[C@H](N)Cc2ccc(O)cc2)[C@@H](C)O)(C(=O)N[C@@H](Cc2ccccc2)C(N)=O)CC1]

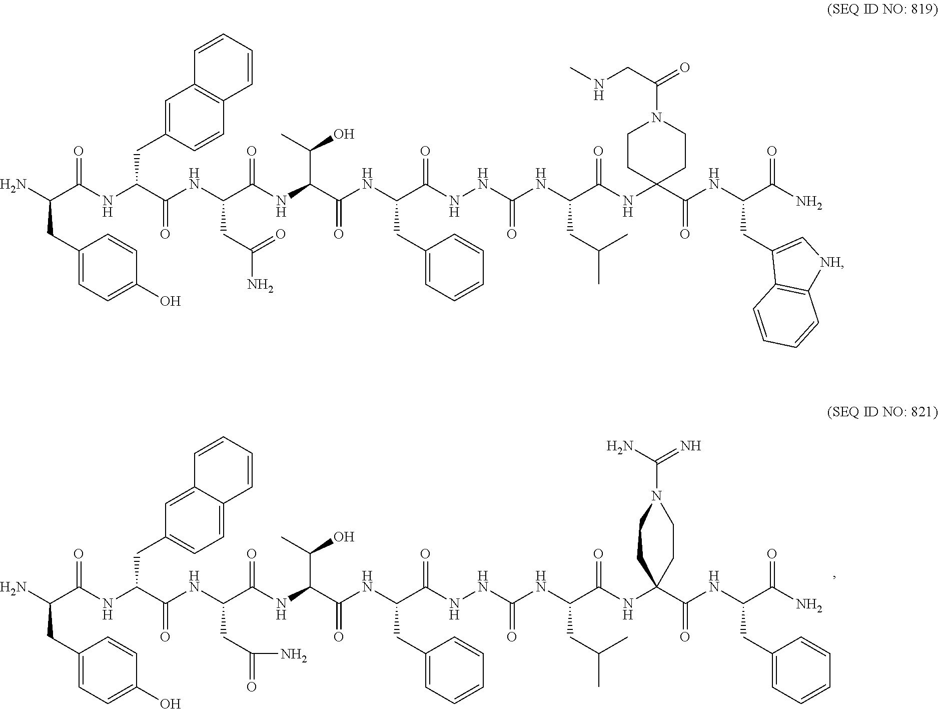

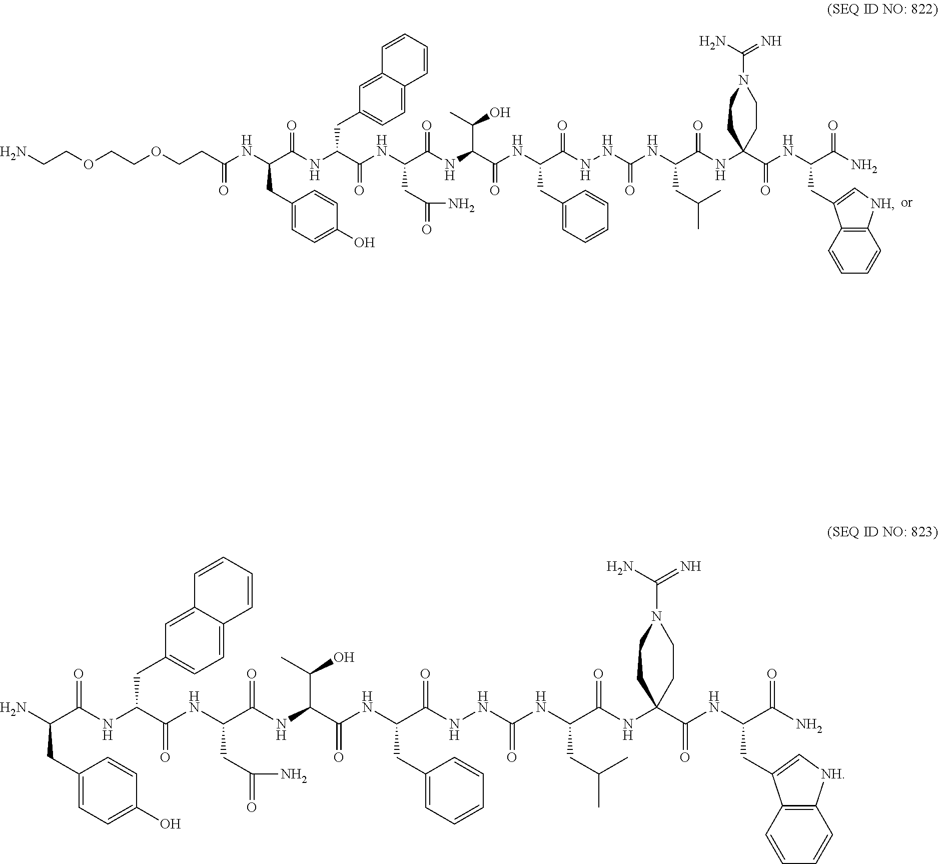

In some embodiments, the compound of Formula (II) is compound 390, or a pharmaceutically acceptable salt thereof; compound 391, or a pharmaceutically acceptable salt thereof; compound 392, or a pharmaceutically acceptable salt thereof; compound 393, or a pharmaceutically acceptable salt thereof; compound 394, or a pharmaceutically acceptable salt thereof; compound 395, or a pharmaceutically acceptable salt thereof; compound 396, or a pharmaceutically acceptable salt thereof; compound 397, or a pharmaceutically acceptable salt thereof; compound 398, or a pharmaceutically acceptable salt thereof; compound 399, or a pharmaceutically acceptable salt thereof; compound 400, or a pharmaceutically acceptable salt thereof; compound 398, or a pharmaceutically acceptable salt thereof; compound 401, or a pharmaceutically acceptable salt thereof; compound 402, or a pharmaceutically acceptable salt thereof; compound 403, or a pharmaceutically acceptable salt thereof; compound 404, or a pharmaceutically acceptable salt thereof; compound 405, or a pharmaceutically acceptable salt thereof; compound 406, or a pharmaceutically acceptable salt thereof; compound 407, or a pharmaceutically acceptable salt thereof; compound 408, or a pharmaceutically acceptable salt thereof; compound 409, or a pharmaceutically acceptable salt thereof; compound 410, or a pharmaceutically acceptable salt thereof; or compound 411, or a pharmaceutically acceptable salt thereof.

In some embodiments, the compound of Formula (II) is compound 402, or a pharmaceutically acceptable salt thereof. In some embodiments, the compound of Formula (II) is compound 403, or a pharmaceutically acceptable salt thereof. In some embodiments, the compound of Formula (II) is compound 404, or a pharmaceutically acceptable salt thereof. In some embodiments, the compound of Formula (II) is compound 405, or a pharmaceutically acceptable salt thereof. In some embodiments, the compound of Formula (II) is compound 406, or a pharmaceutically acceptable salt thereof. In some embodiments, the compound of Formula (II) is compound 407, or a pharmaceutically acceptable salt thereof. In some embodiments, the compound of Formula (II) is compound 408, or a pharmaceutically acceptable salt thereof. In some embodiments, the compound of Formula (II) is compound 409, or a pharmaceutically acceptable saltthereof. In some embodiments, the compound of Formula (II) is compound 410, or a pharmaceutically acceptable salt thereof. In some embodiments, the compound of Formula (II) is compound 411, or a pharmaceutically acceptable salt thereof.

### Further Forms of Compounds

In one aspect, compounds described herein are in the form of pharmaceutically acceptable salts. In addition, the compounds described herein can exist in unsolvated as well as solvated forms with pharmaceutically acceptable solvents such as water, ethanol, and the like. The solvated forms of the compounds presented herein are also considered to be disclosed herein.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. See for example Handbook of Pharmaceutical Salts: Properties, Selection and Use; International Union of Pure and Applied Chemistry, Wiley-VCH 2002; S. M. Berge, L. D. Bighley, D. C. Monkhouse, J. Pharm. Sci. 1977, 66, 1-19; and P. H. Stahl and C. C. Wermuth, editors, Handbook of Pharmaceutical Salts: Properties., Selection and Use, Weinheim/Zürich:Wiley-VCH/VHCA, 2002; which are incorporated herein by reference. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound of Formula (I) with an acid. In some embodiments, the acid is an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to: 1-hydroxy-2-naphthoic acid; 2,2-dichloroacetic acid; 2-hydroxyethanesulfonic acid; 2-oxoglutaric acid; 4-acetamidobenzoic acid; 4-aminosalicyclic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound of Formula (I), is prepared as a chloride salt, sulfate salt, bromide salt, mesylate salt, maleate salt, citrate salt or phosphate salt.

In some embodiments, pharmaceutically acceptable salts are obtained by reacting a compound of Formula (I), with a base. In some cases, compounds described herein coordinate with an organic base, such as, but not limited to, ethanolamine, diethanolamine, triethanolamine, tromethamine, meglumine, N-methylglucamine, dicyclohexylamine, or tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some embodiments, any one of the hydrogen atoms on the organic radicals (e.g., alkyl groups, aromatic rings) of compounds described herein are replaced with deuterium.

In some embodiments, the compounds of Formula (I), possess one or more stereocenters and each stereocenter exists independently in either the R or S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds described herein, are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the formation of diastereomeric salts and separation by recrystallization, or chromatography, or any combination thereof. See for example Jean Jacques, Andre Collet, Samuel H. Wilen, “Enantiomers, Racemates and Resolutions”, John Wiley And Sons, Inc., 1981, which is incorporated herein by reference. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. See for example Design of Prodrugs, Bundgaard, A. Ed., Elsevier, 1985 and Method in Enzymology, Widder, K. et ail., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference.

A “metabolite” of a compound disclosed herein is a derivative of that compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Metabolites of the compounds disclosed herein are optionally identified either by administration of compounds to a host and analysis of tissue samples from the host, or by incubation of compounds with hepatic cells in vitro and analysis of the resulting compounds.

### Synthesis of Compounds

Compounds described herein are synthesized using standard synthetic techniques or using methods known in the art in combination with methods described herein.

Unless otherwise indicated, conventional methods of mass spectroscopy, NMR, and HPLC are employed.

Compounds are prepared using standard organic chemistry techniques such as those described in, for example, March's Advanced Organic Chemistry, 6^{th} Edition, John Wiley and Sons, Inc. Compounds may also be prepared using solid-phase peptide synthesis techniques such as those described in, for example, Solid Phase Peptide Synthesis, 2^{nd} Edition. The Pierce Chemical Co., Rockford, Ill. (1984). Alternative reaction conditions for the synthetic transformations described herein may be employed such as variation of solvent, reaction temperature, reaction time, as well as different chemical reagents and other reaction conditions.

### Peptide Synthesis

A peptide of the present disclosure may be prepared through known methods, including solid-phase peptide synthesis (SPPS). (See, for example, Palomo, Jose M. (2014); “Solid-phase peptide synthesis: An overview focused on the preparation of biologically relevant peptides” RSC Adv. 4 (62): 32658-32672; Krch\hat{n}ák, V; Holladay, Mark W. (2002); “Solid Phase Heterocyclic Chemistry” Chemical Reviews. 102 (1): 61-92; Merrifield, B. (1986-04-18); “Solid phase synthesis” Science. 232 (4748): 341-347; Guillier, F; et al., (2000). “Linkers and Cleavage Strategies in Solid-Phase Organic Synthesis and Combinatorial Chemistry.” Chemical Reviews. 100 (6): 2091-2158; Amblard M, et al., “Methods and protocols of modern solid phase Peptide synthesis.” Mol Biotechnol. 2006 Jul.; 33(3):239-54).

In some embodiments, the solid-phase peptide synthesis is Fmoc solid-phase peptide synthesis. See, for example, Behrendt, R—, et al., (2016) Advances in Fmoc solid-phase peptide synthesis. J. Pept. Sci., 22: 4-27.

SPPS is a common technique for peptide synthesis. Usually, peptides are synthesized from the carbonyl group side (C-terminus) to amino group side (N-terminus) of the amino acid chain in the SPPS method, although peptides are biologically synthesized in the opposite direction in cells. In peptide synthesis, an amino-protected amino acid is bound to a solid phase material or resin (most commonly, low cross-linked polystyrene beads), forming a covalent bond between the carbonyl group and the resin, most often an amido or an ester bond. Then the amino group is deprotected and reacted with the carbonyl group of the next N-protected amino acid. The solid phase now bears a dipeptide. This cycle is repeated to form the desired peptide chain. After all reactions are complete, the synthesized peptide is cleaved from the bead.

The protecting groups for the amino groups mostly used in the peptide synthesis are 9-fluorenylmethyloxycarbonyl group (Fmoc) and t-butyloxycarbonyl (Boc). A number of amino acids bear functional groups in the side chain which must be protected specifically from reacting with the incoming N-protected amino acids. In contrast to Boc and Fmoc groups, these have to be stable over the course of peptide synthesis although they are also removed during the final deprotection of peptides.

An example solid-phase peptide synthesis may be carried out as follows. An esterification reaction occurs between the carboxyl group of a first amino acid (with a protected α-amino group) and the hydroxyl group of a hydroxyl-containing resin. The α-amino protecting group of the first amino acid is removed and a second amino acid is coupled with the first through its carboxyl group (all other functional groups are protected) to form a peptide bond between the first and second amino acids. The α-amino protecting group of the second amino acid is removed and a third amino acid is coupled with the second through its carboxyl group (all other functional groups are protected) to form a peptide bond between the second and third amino acids. These steps are repeated until the peptide of desired length is synthesized. Any remaining functional groups on the peptide chain are then deprotected. The peptide chain can then be cleaved from the resin.

Examples of resins used for SPPS include Merrifield resin, Rink amide resin, Wang resin, Sieber amide resin, MBHA resin, CTC resin, HMBA resin, DHP resin, and PAL resin. In some embodiments, the resin for SPPS is Rink amide resin. In some embodiments, the resin for SPPS is Wang resin. In some embodiments, the resin for SPPS is 2-chlorotrityl resin. In some embodiments, the resin for SPPS is Sieber amide resin.

Examples of α-amino protecting groups include benzyloxycarbonyl (Cbz), tertbutoxycarbonyl (Boc), fluorenylmethoxycarbonyl (Fmoc), an d allyloxycarbonyl (Alloc) groups. In some embodiments, the α-amino protecting group is Fmoc. In some embodiments, the α-amino protecting group can be deprotected using acid, such as hydrofluoric acid or trifluoroacetic acid, in some embodiments, the α-amino protecting group can be deprotected using base, such as piperidine.

Examples of condensation agents used to activate a carboxyl group for an amidification or esterification reaction include HATU, DCC, EDC, BOP, and HBTU. In some embodiments, the condensation agent is HATU.

Examples of acids use to cleave a peptide chain from the resin include TFA.

In some embodiments, compounds are prepared as described in the Examples.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to, delivery via parenteral routes (including injection or infusion, and subcutaneous).

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and contain optional agents as excipients such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use.

### Methods of Treatment

In some embodiments, the methods comprise administering to a subject a therapeutically effective amount of a compound of Formula (I), or a pharmaceutically acceptable salt or solvate thereof. In some embodiments, the compound of Formula (I) or pharmaceutically acceptable salt or solvate thereof is administered in a pharmaceutical composition. In some embodiments, the subject has cancer. In some embodiments, the cancer is a solid tumor. In some embodiments, the subject has a noncancerous tumor. In some embodiments, the subject has an adenoma.

In some embodiments, the treatment is sufficient to reduce or inhibit the growth of the subject's tumor, reduce the number or size of metastatic lesions, reduce tumor load, reduce primary tumor load, reduce invasiveness, prolong survival time, or maintain or improve the quality of life, or combinations thereof.

In some embodiments, provided herein are methods for killing a tumor cell comprising contacting the tumor cell with a compound of Formula (I), or a pharmaceutically acceptable salt or solvate thereof. In some embodiments, the compound of Formula (I), or pharmaceutically acceptable salt or solvate thereof releases a number of alpha particles by natural radioactive decay. In some embodiments, the released alpha particles are sufficient to kill the tumor cell. In some embodiments, the released alpha particles are sufficient to stop cell growth. In some embodiments, the tumor cell is a malignant tumor cell. In some embodiments, the tumor cell is a benign tumor cell. In some embodiments, the method comprises killing a tumor cell with a beta-particle emitting radionuclide. In some embodiments, the method comprises killing a tumor cell with an alpha-particle emitting radionuclide. In some embodiments, the method comprises killing a tumor cell with a gamma-particle emitting radionuclide.

In one aspect, provided herein are methods and compositions for treating cancers. In some embodiments, the cancer is ovarian cancer. In some embodiments, the cancer is breast cancer. In some embodiments, the cancer is endometrial cancer. In some embodiments, the cancer is prostate cancer. In some embodiments, the cancer is renal cell carcinoma. In some embodiments, the cancer is lung cancer.

In one aspect, provided herein are methods and compositions for treating an adenoma.

In one aspect, provided herein are methods and compositions for treating a carcinoma.

In one aspect, provided herein is a method for identifying tissues or organs in a mammal that overexpress KISS1R comprising: (i) administering to the mammal a KISS1R radiopharmaceutical described herein, or a pharmaceutically acceptable salt thereof; and (ii) performing single-photon emission computerized tomography (SPECT) or positron emission tomography (PET) analysis on the mammal. In some embodiments, the method comprises: (i) administering to the mammal a KISS1radiopharmaceutical described herein, or a pharmaceutically acceptable salt thereof; and (ii) performing positron emission tomography (PET) analysis on the mammal.

In some embodiments, the mammal was diagnosed with cancer. In some embodiments, the mammal was diagnosed with ovarian cancer. In some embodiments, the mammal was diagnosed with breast cancer. In some embodiments, the mammal was diagnosed with endometrial cancer. In some embodiments, the mammal was diagnosed with prostate cancer. In some embodiments, the tissues in the mammal that overexpress KISS1R are tumors.

In some embodiments, a KISS1R radiopharmaceutical described herein, or a pharmaceutically acceptable salt thereof are used in a method for in vivo imaging of a subject. In some embodiments, the method includes the steps of:
(i) administering to the mammal KISS1R radiopharmaceutical described herein, or a pharmaceutically acceptable salt thereof;
(ii) waiting a sufficient amount of time to allow the KISS1R radiopharmaceutical, to accumulate at a tissue or cell site to be imaged; and
(iii) imaging the cells or tissues with a non-invasive imaging technique.

In some embodiments, the non-invasive imaging technique is single-photon emission computerized tomography (SPECT) or positron emission tomography (PET) analysis. In some embodiments, the non-invasive imaging technique is single-photon emission computerized tomography (SPECT). In some embodiments, the non-invasive imaging technique is selected from positron emission tomography imaging, or positron emission tomography with computed tomography imaging, and positron emission tomography with magnetic resonance imaging.

In some embodiments, the methods comprise administering to a subject a therapeutically effective amount of a compound of Formula (II), or a pharmaceutically acceptable salt or solvate thereof. In some embodiments, the compound of Formula (II), or pharmaceutically acceptable salt or solvate thereof is administered in a pharmaceutical composition. In some embodiments, the pharmaceutical composition is formulated for administration to a mammal by oral administration. In some embodiments, the subject has cancer. In some embodiments, the cancer is breast cancer. In some embodiments, the cancer is prostate cancer. In some embodiments, the subject has an endocrine condition. In some embodiments, the subject has is polycystic ovary syndrome (PCOS). In some embodiments, the subject suffers from infertility.

In one aspect, provided herein are methods and compositions for treating an endocrine condition. In some embodiments, the endocrine condition is polycystic ovary syndrome (PCOS). In some embodiments, the endocrine condition is infertility.

In one aspect, provided herein are methods and compositions for treating cancer. In some embodiments, the cancer is breast cancer. In some embodiments, the cancer is prostate cancer.

In one aspect, provided herein are methods and compositions for treating infertility.

### Methods of Dosing and Treatment Regimens

In one embodiment, the KISS1R radiopharmaceutical described herein, or a pharmaceutically acceptable salt thereof, are used in the preparation of medicaments for the treatment of tumors in a mammal. Methods for treating any of the diseases or conditions described herein in a mammal in need of such treatment, involves administration of pharmaceutical compositions that include at least one compound of Formula (I), or a pharmaceutically acceptable salt thereof, in therapeutically effective amounts to said mammal.

In certain embodiments, the compositions containing the compound(s) described herein are administered for diagnostic and/or therapeutic treatments.

The amount of a given agent that corresponds to such an amount varies depending upon factors such as the particular conjugate, specific cancer or tumor to be treated (and its severity), the identity (e.g., weight, sex) of the subject or host in need of treatment, but nevertheless is determined according to the particular circumstances surrounding the case, including, e.g., the specific conjugate being administered, the route of administration, the condition being treated, and the subject or host being treated. Optimal doses are generally determined using experimental models and/or clinical trials. The optimal dose depends upon the body mass, weight, or blood volume of the subject.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans.

The amount of a compound of Formula (I), or pharmaceutically acceptable salts thereof, that are administered are sufficient to deliver a therapeutically effective dose to the particular subject. In some embodiments, dosages of a compound of Formula (I), are between about 0.1 pg and about 50 mg per kilogram of body weight, 1 μg and about 50 mg per kilogram of body weight, or between about 0.1 and about 10 mg/kg of body weight. Therapeutically effective dosages can also be determined at the discretion of a physician. By way of example only, the dose of a compound of Formula (I), or a pharmaceutically acceptable salt thereof described herein for methods of treating a disease as described herein is about 0.001 mg/kg to about 1 mg/kg body weight of the subject per dose. In some embodiments, the dose is about 0.001 mg to about 1000 mg per dose for the subject being treated. In some embodiments, a compound of Formula (I), or a pharmaceutically acceptable salt thereof described herein is administered to a subject at a dosage of from about 0.01 mg to about 500 mg, from about 0.01 mg to about 100 mg, or from about 0.01 mg to about 50 mg.

In some embodiments, a compound of Formula (I), or a pharmaceutically acceptable salt thereof described herein is administered to a subject at a dosage of about 0.01 picomole to about 1 mole, about 0.1 picomole to about 0.1 mole, about 1 nanomole to about 0.1 mole, or about 0.01 micromole to about 0.1 millimole.

In some embodiments, a compound of Formula (I), or a pharmaceutically acceptable salt thereof described herein is administered to a subject at a dosage of about 0.01 Gbq to about 1000 Gbq, about 0.5 Gbq to about 100 Gbq, or about 1 Gbq to about 50 Gbq.

In some embodiments, the dose is administered once a day, 1 to 3 times a week, 1 to 4 times a month, or 1 to 12 times a year.

In any of the aforementioned aspects are further embodiments in which the effective amount of the KISS1R radiopharmaceutical described herein, or a pharmaceutically acceptable salt thereof, is: (a) systemically administered to the mammal; and/or (b) intravenously administered to the mammal; and/or (c) administered by injection to the mammal.

In certain instances, it is appropriate to administer at least one KISS1R radiopharmaceutical described herein, or a pharmaceutically acceptable salt thereof, in combination with one or more other therapeutic agents.

### Certain Terminology

Unless otherwise stated, the following terms used in this application have the definitions given below. The use of the term “including” as well as other forms, such as “include,” “includes,” and “included,” is not limiting. The section headings used herein are for organizational purposes only and are not to be construed as limiting the subject matter described.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, C₁-Cₓ includes C₁-C₂, C₁-C₃ . . . C₁-Cₓ, By way of example only, a group designated as “C₁-C₆” indicates that there are one to six carbon atoms in the moiety, i.e., groups containing 1 carbon atom, 2 carbon atoms, 3 carbon atoms, 4 carbon atoms, 5 carbon atoms or 6 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e., a —C₁-C₁₀ alkyl. Whenever it appears herein, a numerical range such as “1 to 10” refers to each integer in the given range; e.g., “1 to 10 carbon atoms” means that the alkyl group consists of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a —C₁-C₆ alkyl, in one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl. In some embodiments, the alkyl group is an “alkenyl” or “alkynyl” group.

An “alkylene” group refers to a divalent alkyl radical. Any of the above-mentioned monovalent alkyl groups may be an alkylene by abstraction of a second hydrogen atom from the alkyl. In some embodiments, an alkylene is a —C₁-C₆alkylene. In other embodiments, an alkylene is a —C₁-C₄ alkylene. Typical alkylene groups include, but are not limited to: —CH₂—, —CH₂CH—, —CH₂CH₂CH₂—, —CH₂CH₂CH₂CH₂—, and the like. In some embodiments, an alkylene is —CH₂—. In some embodiments, an alkylene is —CH₂—CH₂—.

An “alkoxy” group refers to an (alkyl)O-group, where alkyl is as defined herein.

The term “alkenyl” refers to a type of alkyl group in which at least one carbon-carbon double bond is present. In one embodiment, an alkenyl group has the formula: —C(R)═CR₂, wherein R refers to the remaining portions of the alkenyl group, which may be the same or different. In some embodiments, each R is independently H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portion of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, and —CH₂C≡CH.

The term “heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. In some embodiments, the “heteroalkyl” group has 2 to 10 atoms in the backbone, which include a combination of carbon atoms and heteroatoms (e.g N, O, S), i.e., a 2 to 10-membered heteroalkyl. In some embodiments, the heteroalkyl is attached to the rest of the molecule at a carbon atom of the heteroalkyl. In one embodiment, a heteroalkyl is a 2 to 8 membered heteroalkyl.

A “heteroalkylene” group refers to a divalent alkyl radical derived from heteroalkyl, as exemplified, but not limited by, —CH₂—CH₂—O—C₂—CH₂— and —CH₂—O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(═O)O— represents both —C(═O)O— and —OC(═O)—. Additionally, the formula —C(═O)NH— represents both —C(═O)NH— and —NHC(═O)—.

The term “carbocyclic” or “carbocycle” refers to a ring or ring system where the atoms formingthe backbone ofthe ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀ aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e., skeletal atoms) is a carbon atom. In some embodiments, cycloalkyls are spirocyclic or bridged cycloalkyls. In some embodiments, cycloalkyls are optionally fused with an aromatic ring, and the point of attachment is at a carbon that is not an aromatic ring carbon atom. Cycloalkyl groups include groups having from 3 to 12 ring atoms. In some embodiments, cycloalkyl groups are selected from cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[0.1.1]pentyl. In some embodiments, a cycloalkyl is a C₃-C₆ cycloalkyl. In some embodiments, a cycloalkyl is a C₃-C₄ cycloalkyl. In some embodiments, a cycloalkyl is a C₅-C₆ cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a —C₁-C₆ fluoroalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 12 atoms in its ring system, and with the proviso that any ring does not contain two adjacent O or S atoms. Non-aromatic heterocyclic groups (also known as heterocycloalkyls) include rings having 3 to 12 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1l-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (A attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4N atoms in the ring. In some embodiments, a heteroaryl contains 1-4N atoms in the ring. In some embodiments, a heteroaryl contains 0-4N atoms, 0-1 O atoms, and 0-1 S atoms in the ring. In some embodiments, a heteroaryl contains 1-4N atoms, 0-1 O atoms, and 0-1 S atoms in the ring. In some embodiments, a heteroaryl contains 1 O atom. In some embodiments, a heteroaryl contains 1 S atom in the ring. In some embodiments, heteroaryl is a 5 to 10-membered heteroaryl. In some embodiments, a monocyclic heteroaryl is a 5 to 6 membered heteroaryl. In some embodiments, a monocyclic heteroaryl is a 5-membered heteroaryl. In some embodiments, a monocyclic heteroaryl is a 6-membered heteroaryl. In some embodiments, bicyclic heteroaryl is a 10-membered heteroaryl.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a 3 to 12 membered heterocycloalkyl. In another aspect, a heterocycloalkyl is a 5 to 10-membered heterocycloalkyl. In some embodiments, a heterocycloalkyl is a 5-membered heterocycloalkyl. In some embodiments, a heterocycloalkyl is a 6-membered heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 1-4 nitrogen (N) atoms in the ring. In some embodiments, a heterocycloalkyl contains 0-2N atoms, 0-2 oxygen (O) atoms and 0-1 sulfur (S) atoms in the ring.

The term “bond” or “single bond” refers to a chemical bond between two atoms, or two moieties when the atoms joined by the bond are considered to be part of a larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃), —OH, —CO₂H, —CO₂(C₁-C₄ alkyl), —C(═O)NH₂, —C(═O)NH(C₁-C₄ alkyl, —C(═O)N(C₁-C₄ alkyl)₂, —S(═O)₂NH₂, —S(═O)₂NH(C₁-C₄alkyl), —S(═O)₂N(C₁-C₄ alkyl)₂, —C₁-C₄, alkyl, C₃-C₆ cycloalkyl, —C₁-C₄ fluoroalkyl, —C₁-C₄heteroalkyl, —C₁-C₄ alkoxy, —C₁-C₄ fluoroalkoxy, —SC₁-C₄alkyl, —S(═O)C₁-C₄alkyl, and —S(═O)₂C₁-C₄ alkyl. In some embodiments, optional substituents are independently selected from halogen. —CN, —NH₂, —OH, ˜NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering,” “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion). Those of skill in the art are familiar with administration techniques that can be employed with the compounds and methods described herein.

The terms “co-administration” or the like, as used herein, are meant to encompass administration of the selected therapeutic agents to a single patient and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

Chemical structures depicted herein include all stereochemical forms of the structure, unless otherwise stated.

The term “peptide” as used herein refers to a compound comprising two or more amino acids in a serial array, linked through peptide bonds. The amino acids making up the polypeptide may be naturally derived, or may be synthetic.

The term “amino acid” as used herein refers to both natural and unnatural amino acids. The term “unnatural amino acid” as used herein refers to an amino acid that is not part of the 20 amino acids that occur naturally in protein.

As used herein, amino acid residue refers to an amino acid formed upon chemical digestion (hydrolysis) of a polypeptide at its peptide linkages. The amino acid residues described herein are, in certain embodiments, in the “L” isomeric form. Residues in the “D” isomeric form can be substituted for any “L” amino acid residue, as long as the desired functional property is retained by the polypeptide. “—NH₂” refers to the free amino group present at the amino terminus of a polypeptide. “—CO₂H” refers to the free carboxy group present at the carboxyl terminus of a polypeptide. In keeping with standard polypeptide nomenclature described in J Biol. Chem, 243:355259 (1969) and adopted at 37 C.F.R. §§ 1.821-1.822, abbreviations for amino acid residues are shown in the following Table B:

TABLE B
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino terminus to carboxyl terminus. In addition, the phrase “amino acid residue” is broadly defined to include the amino acids listed in the Table of Correspondence and modified and unusual amino acids, such as those referred to in 37 C.F.R. §§ 1.821-1.822, and incorporated herein by reference. Furthermore, it should be noted that a dash at the beginning or end of an amino acid residue sequence indicates a peptide bond to a further sequence of one or more amino acid residues or to an amino terminal group such as —NH₂ or to a carboxyl terminal group such as —CO₂H.

In a peptide, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub co., p. 224). Such substitutions can be made in accordance with those set forth in Table C as Table C.

TABLE C

| Original residue | Conservative substitution |
|---|---|
| Ala (A) | Gly; Ser |
| Arg (R) | Lys |
| Asn (N) | Gln; His |
| Asp (D) | Glu |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Representative amino acid side chains are shown in Table D.

TABLE D
Representative amino acid side chains

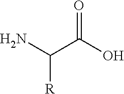

—H
Glycine (Gly)

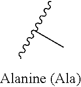

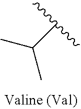 [SMILES: *C(C)C]

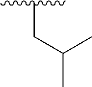 [SMILES: *CC(C)C]

Leucine (Leu)

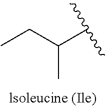 [SMILES: *C(C)CC]

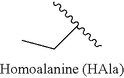 [SMILES: *CC]

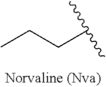 [SMILES: *CCC]

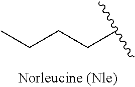 [SMILES: *CCCC]

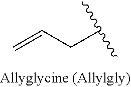 [SMILES: *CC=C]

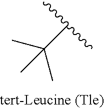 [SMILES: *C(C)(C)C]

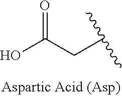 [SMILES: *CC(=O)O]

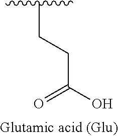 [SMILES: *CCC(=O)O]

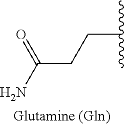 [SMILES: *CCC(N)=O]

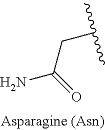 [SMILES: *CC(N)=O]

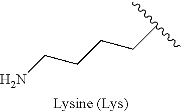 [SMILES: *CCCCN]

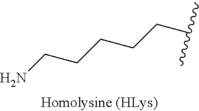 [SMILES: *CCCCCN]

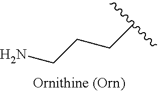 [SMILES: *CCCN]

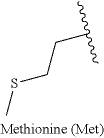 [SMILES: *CCSC]

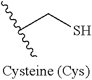 [SMILES: *CS]

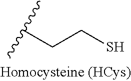 [SMILES: *CCS]

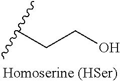 [SMILES: *CCO]

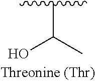 [SMILES: *C(C)O]

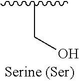 [SMILES: *CO]

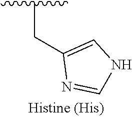 [SMILES: *Cc1c[nH]cn1]

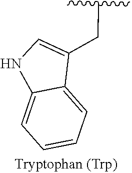 [SMILES: *Cc1c[nH]c2ccccc12]

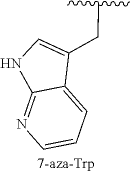 [SMILES: *Cc1c[nH]c2ncccc12]

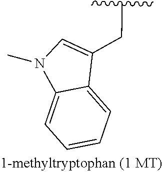 [SMILES: *Cc1cn(C)c2ccccc12]

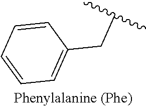 [SMILES: *Cc1ccccc1]

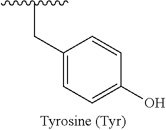 [SMILES: *Cc1ccc(O)cc1]

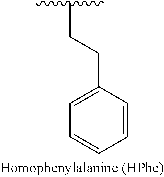 [SMILES: *CCc1ccccc1]

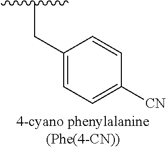 [SMILES: *Cc1ccc(C#N)cc1]

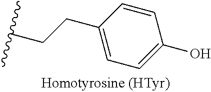 [SMILES: *CCc1ccc(O)cc1]

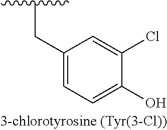 [SMILES: *Cc1ccc(O)c(Cl)c1]

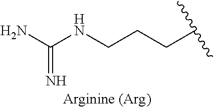 [SMILES: *CCCNC(=N)N]

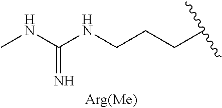 [SMILES: *CCCNC(=N)NC]

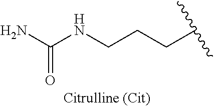 [SMILES: *CCCNC(N)=O]

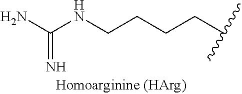 [SMILES: *CCCCNC(=N)N]

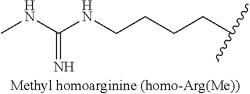 [SMILES: *CCCCNC(=N)NC]

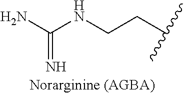 [SMILES: *CCNC(=N)N]

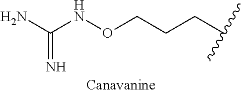 [SMILES: *CCCONC(=N)N]

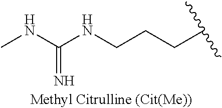 [SMILES: *CCCNC(=N)NC]

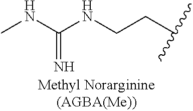 [SMILES: *CCNC(=N)NC]

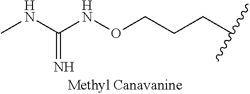 [SMILES: *CCCONC(=N)NC]

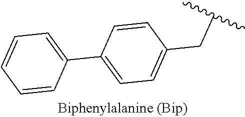 [SMILES: *Cc1ccc(-c2ccccc2)cc1]

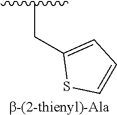 [SMILES: *Cc1cccs1]

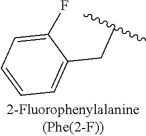 [SMILES: *Cc1ccccc1F]

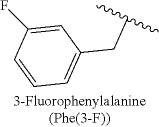 [SMILES: *Cc1cccc(F)c1]

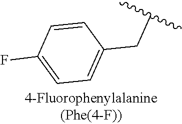 [SMILES: *Cc1ccc(F)cc1]

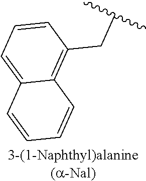 [SMILES: *Cc1cccc2ccccc12]

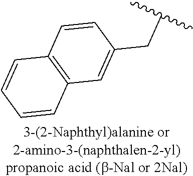 [SMILES: *Cc1ccc2ccccc2c1]

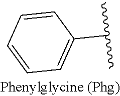 [SMILES: *c1ccccc1]

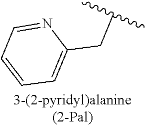 [SMILES: *Cc1ccccn1]

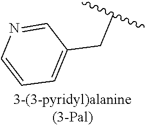 [SMILES: *Cc1cccnc1]

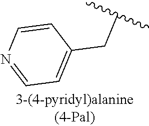 [SMILES: *Cc1ccncc1]

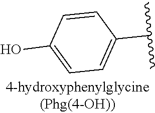 [SMILES: *c1ccc(O)cc1]

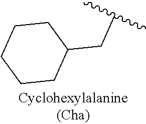 [SMILES: *CC1CCCCC1]

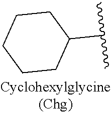 [SMILES: *C1CCCCC1]

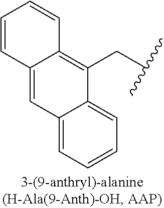 [SMILES: *Cc1c2ccccc2cc2ccccc12]

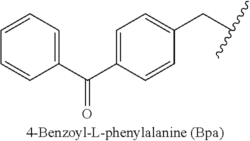 [SMILES: *Cc1ccc(C(=O)c2ccccc2)cc1]

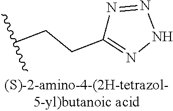 [SMILES: *CCc1nn[nH]n1]

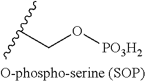

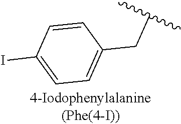 [SMILES: *Cc1ccc(I)cc1]

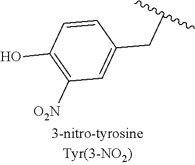 [SMILES: *Cc1ccc(O)c([N+](=O)[O-])c1]

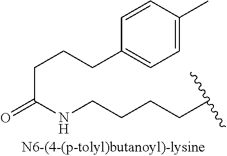 [SMILES: *CCCCNC(=O)CCCc1ccc(C)cc1]

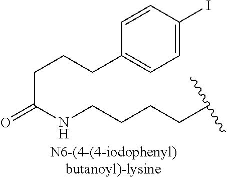 [SMILES: *CCCCNC(=O)CCCc1ccc(I)cc1]

Additional amino acids are shown in Tables E and F.

TABLE E
Representative cyclic and unnatural amino acids

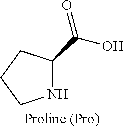

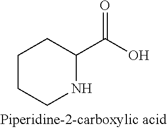

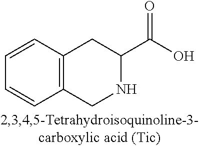 [SMILES: O=C(O)C1Cc2ccccc2CN1]

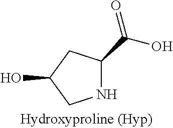

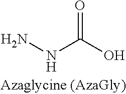

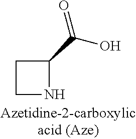

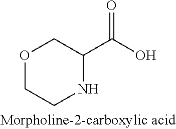

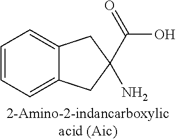 [SMILES: NC1(C(=O)O)Cc2ccccc2C1]

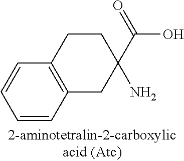 [SMILES: NC1(C(=O)O)CCc2ccccc2C1]

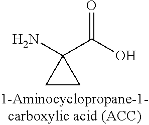

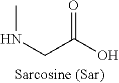

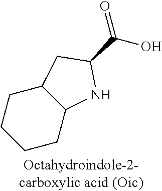 [SMILES: O=C(O)[C@@H]1CC2CCCCC2N1]

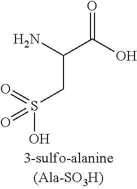

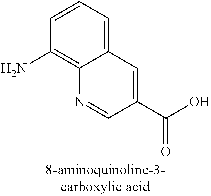 [SMILES: Nc1cccc2cc(C(=O)O)cnc12]

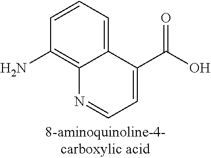 [SMILES: Nc1cccc2c(C(=O)O)ccnc12]

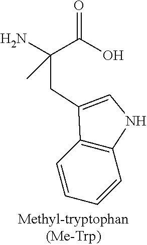 [SMILES: CC(N)(Cc1c[nH]c2ccccc12)C(=O)O]

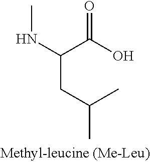

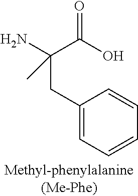

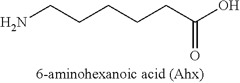

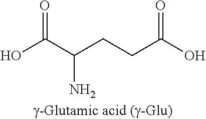

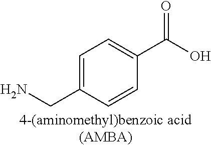 [SMILES: NCc1ccc(C(=O)O)cc1]

TABLE F
Representative β-amino acids

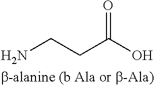

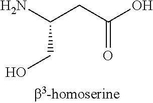

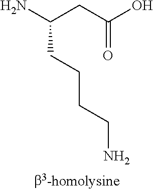 [SMILES: NCCCC[C@H](N)CC(=O)O]

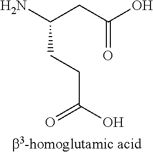 [SMILES: N[C@@H](CCC(=O)O)CC(=O)O]

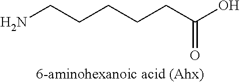

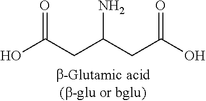

### Numbered Embodiments

Embodiment 1. A compound of Formula (I), or a pharmaceutically acceptable salt thereof:

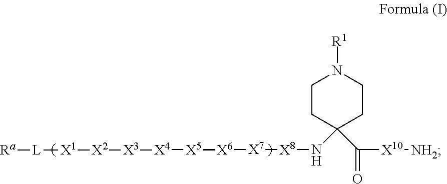

wherein:
R^{a} is a chelating moiety or a radionuclide complex thereof;
L is an optional linker that is attached to any one of X¹, X², X X⁴, X⁵, X⁶, or X⁷ or
L is attached to X⁸ if X¹, X², X³, X⁴, X⁵, X⁶, and X⁷ are absent; and
R¹ is H,

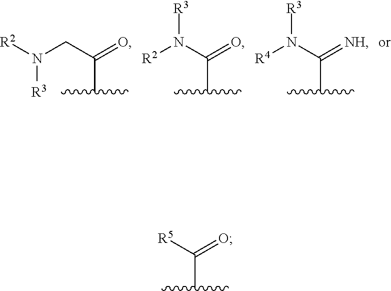

R² is C₁-C₈ alkyl, substituted or unsubstituted heteroalkyl, —(CHR⁶)ₙ-heterocycloalkyl, —(CHR⁶)ₙ-aryl, —(CHR⁶)ₙ-heteroaryl, —C(═O)—(CHR⁶)ₙ-aryl, or —C(═O)NH—(CHR⁶)ₙ-aryl; wherein C₁-C₆ alkyl is optionally substituted with R⁷, and wherein the heterocycloalkyl, aryl, or heteroaryl are each independently optionally substituted with R⁷, R⁸, R⁹, R¹⁰, and R¹¹;
R³ is H or (C₁-C₄ alkyl;
R⁴ is H, C₁-C₄ alkyl, or R²;
R⁵ is substituted or unsubstituted alkyl, substituted or unsubstituted heteroalkyl, or —(CHR⁶)ₙ-aryl; wherein aryl is optionally substituted with R⁷, R⁸, R⁹, R¹⁰, and R¹¹;
each R⁶ is independently H, F, —CR₃, ˜NH₂, or —OH;
R⁷, R⁸, R⁹, R¹⁰, and R¹¹ are each independently selected from H, F, Cl, Br, I, —OH, —O—C₁-C₄ alkyl, —NH₂, —NHC₁-C₄alkyl, —N(C₁-C₄ alkyl)₂, —CN, —CO₂H, —CO₂C₁-C₄ alkyl, —C₁-C₆ alkyl, —C₁-C₆ fluroroalkyl or —C₃-C₆ cycloalkyl;
   n is 0, 1, 2, 3, 4, 5, or 6;
X is absent, tyrosine (Tyr), glycine (Gly), sarcosine (Sar), alanine (Ala), aspartic acid (Asp), lysine (Lys), phenylalanine (Phe), 3-(3-pyridyl)alanine (3-Pal), threonine (Thr), methionine (Met), 4-iodophenylalanine (Phe(4-I)), N6-(4-(p-tolyl)butanoyl)-lysine, N6-(4-(4-iodophenyl)butanoyl)-lysine, or γ-glutamic acid (γ-Glu));
X² is absent, asparagine (Asn), glutamine (Gln), aspartic acid (Asp), glutamic acid (Glu), serine (Ser), histidine (His), alanine (Ala), sarcosine (Sar), tyrosine (Tyr), proline (Pro), hydroxyproline (Hyp), azetidine-2-carboxylic acid (Aze), 2,3,4,5-tetrahydroisoquinoline-3-carboxylic acid (Tic), phenylalanine (Phe), 3-(2-pyridyl)alanine (2-Pal), 3-(3-pyridyl)alanine (3-Pal), or 3-(4-pyridyl)alanine (4-Pal);
X³ is absent, tryptophan (Trp), serine (Ser), leucine (Leu), isoleucine (Ile), phenylalanine (Phe), 4-iodophenylalanine (Phe(4-I)), 3-(2-pyridyl)alanine (2-Pal), 3-(3-pyridyl)alanine (3-Pal), 3-(4-pyridyl)alanine (4-Pal), 2-amino-3-(naphthalen-2-yl)propanoic acid (H-2-NAL-OH), lysine (Lys), asparagine (Asn), glutamine (Gln), aspartic acid (Asp), glutamic acid (Glu), arginine (Arg), methyl arginine (Arg(Me)), norarginine (AGBA), methyl norarginine(AGBA(Me)), homoarginine(Harg), methyl homoarginine (Harg(Me)), citrulline (Cit), methyl citrulline (Cit(Me)), canavanine, methyl-canavanine, glycine (Gly), alanine (Ala), sarcosine (Sar), tyrosine (Tyr), cyclohexylalanine (Cha), 3-(1-naphthyl)alanine (α-Nal), 3-(2-naphthyl)alanine (β-Nal), threonine (Thr), proline (Pro), hydroxyproline (Hyp), tetrahydroisoquinoline-3-carboxylic acid (Tic), O-phospho-serine (SOP), 2-amino-4-(2H-tetrazol-5-yl)butanoic acid, 3-glutamic acid, 8-aminoquinoline-3-carboxylic acid, biphenylalanine (Bip), 4-benzoylphenylalanine (Bpa), or 3-(9-anthryl)-alanine (H-Ala(9-Anth)-OH or AAP);
X⁴ is absent, asparagine (Asn), glutamine (Gln), aspartic acid (Asp), glutamic acid (Glu), tryptophan (Trp), glycine (Gly), tyrosine (Tyr), alanine (Ala), sarcosine (Sar), or arginine (Arg);
X⁵ is absent, serine (Ser), threonine (Thr), lysine (Lys), asparagine (Asn), glutamine (Gln), aspartic acid (Asp), glutamic acid (Gln), glycine (Gly), alanine (Ala), or sarcosine (Sar);
X⁶ is absent, phenylalanine (Phe), alpha-methylphenylalanine (α-Me-Phe), N-methylphenylalanine (N-Me-Phe), 2-fluorophenylalanine (2-F-Phe), 3-fluorophenylalanine (3-F-Phe), 4-fluorophenylalanine (4-F-Phe), 4-iodophenylalanine (Phe(4-I)), 2-amino-2-indancarboxylic acid (Aic), biphenylalanine (Bip), (β-(2-thienyl)-Ala), tryptophan (Trp), 2-aminotetralin-2-carboxylic acid (Atc), 3-(2-thienyl)-alanine, 3-(4-pyridyl)alanine (4-Pal), cyclohexylalanine (Cha), or tyrosine (Tyr);
X⁷ is absent, glycine (Gly), aza-glycine (aza-Gly), alanine (Ala), N-methylglycine (Sar), or 1-aminocyclopropane-1-carboxylic acid (ACC);
X⁸ is leucine (Leu), norvaline (Nva), valine (Val), isoleucine (Ile), homoalanine (Hala), tryptophan (Trp), phenylalanine (Phe), or phenylglycine (Phg);
or —X⁷—X⁸— is

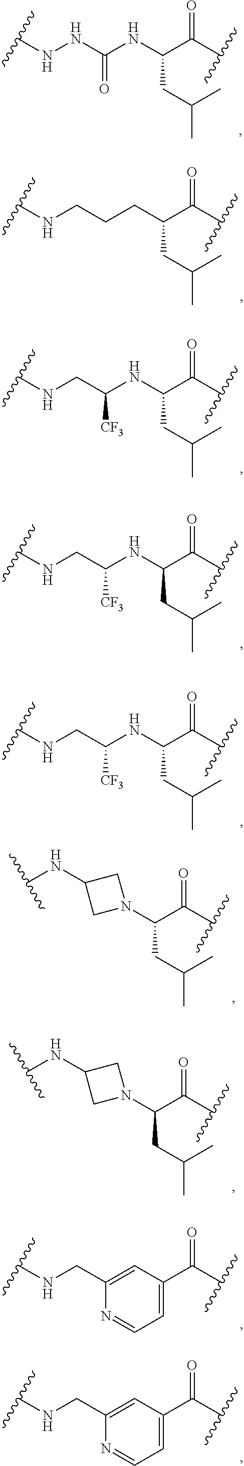

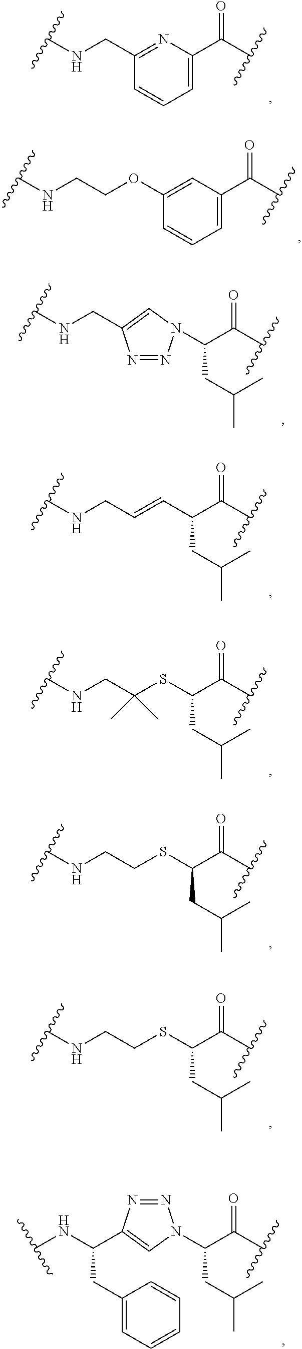

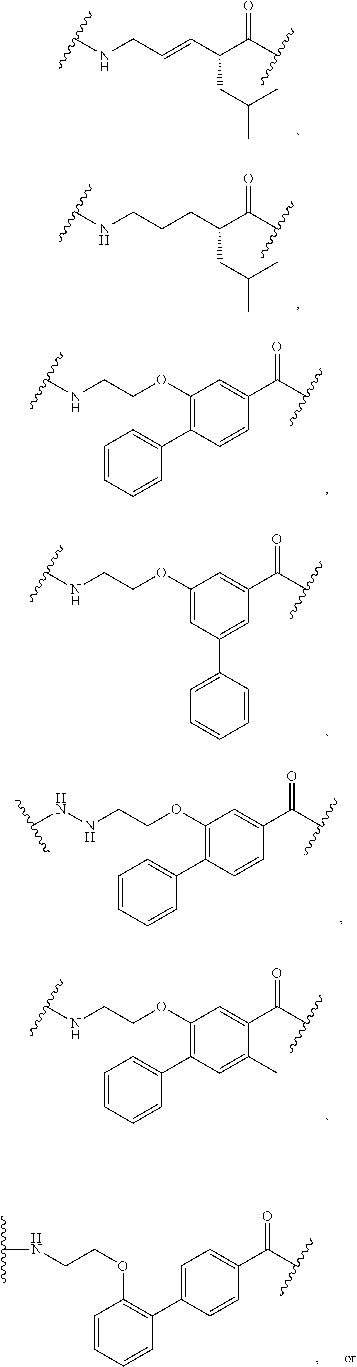

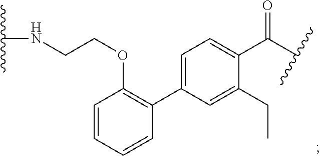 [SMILES: *NCCOc1ccccc1-c1ccc(C(*)=O)c(CC)c1]

or —X⁶—X⁷—X⁸— is

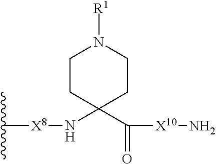

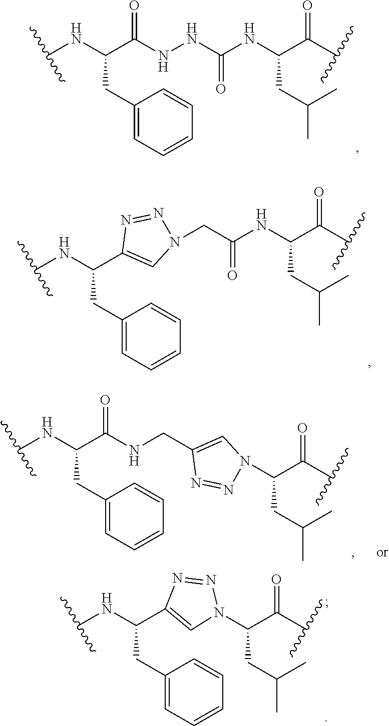

X¹⁰ is tryptophan (Trp), 1-methyltryptophan (1MT), tyrosine (Tyr), phenylalanine (Phe), 4-cyano phenylalanine (Phe(4-CN)), 3-(4-pyridyl)alanine (4-Pal), leucine (Leu), phenylglycine (Phg), cyclohexylalanine (Cha), 3-(1-naphthyl)alanine (t-Nal), 3-(2-naphthyl)-alanine (β-Nal), histidine (His), or 3-nitro-tyrosine (Tyr(3-NO₂))
wherein the N-terminal amino acid or the compound of Formula (I) is optionally substituted with —C(═O)—C₁-C₂₀ alkyl, —C(═O)—(CH₂CH₂O)_{y}—CHCH₂—R¹⁵, C₁-C₂₀ alkyl, N-hexadecanoyl-Glu, C₄-C₂₀ polyethylene glycol, a saccharide, —R¹⁶, —C((CH₂CH₂O)ₓ—CH₃, —C(═O)—(CH₂CH₂O)ₓ—H, —C(═O)—CH₂CH₂CH(COOH)—R¹⁵, —C(═O)—(CH₂)₂R¹⁹, or —C(═O)CH₂NHCH₂R¹⁹;
R¹⁵ is selected from —OR¹⁶, —N(R¹⁶)₂, —C(═O)OR¹⁶, or —C(═O)N(R¹⁶)₂;
each R¹⁶ is independently H, —C₁-C₆ alkyl, —C(═O)—(CH₂)ᵥR¹⁹, —C(═O)CH₂NHCH₂R¹⁹,
or a saccharide or derivative thereof;
R¹⁹ is 4-iodophenylene, 4-methylphenylene, or 3-fluoro-4-methylphenylene;
y is 0, 1, 2, 3, 4, 5, 6, 7, 8, 9, or 10;
x is an integer from 1 and 25; and
v is 1, 2, 3, or 4;
   wherein any free —NH— of a peptide bond is optionally independently substituted with —CH₃ or —CH₂CH₃; and
   wherein any alpha position of an amino acid is optionally independently substituted with —C-3 or —C-₂CH₃.

Embodiment 2. The compound of embodiment 1, or a pharmaceutically acceptable salt thereof, wherein:
X¹ is absent, tyrosine (Tyr), glycine (Gly), sarcosine (Sar), alanine (Ala), aspartic acid (Asp), lysine (Lys), phenylalanine (Phe), 3-(3-pyridyl)alanine (3-Pal), threonine (Thr), methionine (Met), 4-iodophenylalanine (Phe(4-I)), N6-(4-(p-tolyl)butanoyl)-lysine or N6-(4-(4-iodophenyl)butanoyl)-lysine, or γ-glutamic acid (γ-Glu);
X² is absent, asparagine (Asn), glutamine (Gln), aspartic acid (Asp), glutamic acid (Glu), serine (Ser), D-histidine (His), alanine (Ala), sarcosine (Sar), tyrosine (Tyr), proline (Pro), hydroxyproline (Hyp), azetidine-2-carboxylic acid (Aze), 2,3,4,5-tetrahydroisoquinoline-3-carboxylic acid (Tic), phenylalanine (Phe), 3-(2-pyridyl)alanine (2-Pal), 3-(3-pyridyl)alanine (3-Pal), or 3-(4-pyridyl)alanine (4-Pal);
X³ is absent, tryptophan(T p), serine (Ser), leucine(Leu), isoleucine (Ile), phenylalanine (Phe), 4-iodophenylalanine (Phe(4-I)), 3-(2-pyridyl)alanine (2-Pal), 3-(3-pyridyl)alanine (3-Pal), 3-(4-pyridyl)alanine (4-Pal), 2-amino-3-(naphthalen-2-yl)propanoic acid (H-2-NAL-OH), lysine (Lys), asparagine (Asn), glutamine (Gln), aspartic acid (Asp), glutamic acid (Glu), arginine (Arg), methyl arginine (Arg(Me)), norarginine (AGBA), methyl norarginine (AGBA(Me)), homoarginine (Harg), methyl homoarginine (Harg(Me)), citrulline (Cit), methyl citrulline (Cit(Me)), canavanine, methyl-canavanine, glycine (Gly), alanine (Ala), sarcosine (Sar), tyrosine (Tyr), cyclohexylalanine (Cha), 3-(1-naphthyl)alanine (α-Nal), 3-(2-naphthyl)alanine (β-Nal), threonine (Thr), proline (Pro), hydroxyproline (Hyp), tetrahydroisoquinoline-3-carboxylic acid (Tic), O-phospho-serine (SOP), 2-amino-4-(2H-tetrazol-5-yl)butanoic acid, beta-glutamic acid (bGlu), 8-aminoquinoline-3-carboxylic acid, biphenylalanine (Dip); 4-benzoylphenylalanine (Bpa), or 3-(9-anthryl)-alanine(H-Ala(9-Anth)-OH or AAP);
X⁴ is absent, -asparagine (−Asn), glutamine (Gln), aspartic acid (Asp), glutamic acid (Glu), tryptophan (Trp), glycine (Gly), alanine (Ala), sarcosine ( ), or arginine (Arg); and
X⁵ is absent, serine (Ser), threonine (Thr), lysine (Lys), asparagine (−Asn), glutamine (Gln), aspartic acid (Asp), glutamic acid (Glu), glycine (Gly), alanine (Ala), or sarcosine (Sar).

Embodiment 3. The compound of embodiment 1, or a pharmaceutically acceptable salt thereof, wherein:
X¹ is absent, tyrosine (Tyr), or 3-(3-pyridyl)alanine (3-Pal));
X² is absent, asparagine (Asn), glutamine (Gln), serine (Ser), D-histidine (His), or phenylalanine (Phe);
X³ is absent, tryptophan (Trp), isoleucine (fie), 3-(4-pyridyl)alanine (4-Pal), lysine (Lys), aspartic acid (Asp), glutamic acid (Glu), glycine (Gly), alanine (Ala), cyclohexylalanine (Cha), (Hyp), biphenylalanine (Bip); 4-benzoylphenylalanine (Bpa), or 3-(9-anthryl)-alanine (AAP);
X⁴ is absent, asparagine (Asn), or glutamine (Gln); and
X⁵ is absent, serine (Ser), threonine (Thr), glycine (Gly), or alanine (Ala).

Embodiment 4. The compound of embodiment 1, or a pharmaceutically acceptable salt thereof, wherein:
X¹ is D-tyrosine (D-Tyr);
X² is absent;
X³ is D-tryptophan (D-Trp), biphenylalanine (Bip); 4-benzoylphenylalanine (Bpa), or 3-(9-anthryl)-alanine (AAP);
X⁴ is asparagine (Asn); and
X⁵ is serine (Ser) or threonine (Thr).

Embodiment 5. The compound of any one of embodiments 1-4, or a pharmaceutically acceptable salt thereof, wherein:
X⁸ is Leu, Nva, lie, Hala, or Phe; and
X¹⁰ is Trp, 1MT, Tyr, 4-Pal, Phe(4-CN), or Phe.

Embodiment 6. The compound of any one of embodiments 1-4, or a pharmaceutically acceptable salt thereof, wherein:

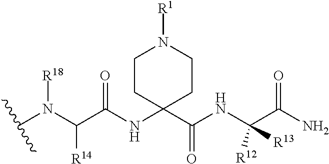

wherein,
R¹⁸ is H or —CH₃;
R¹² is

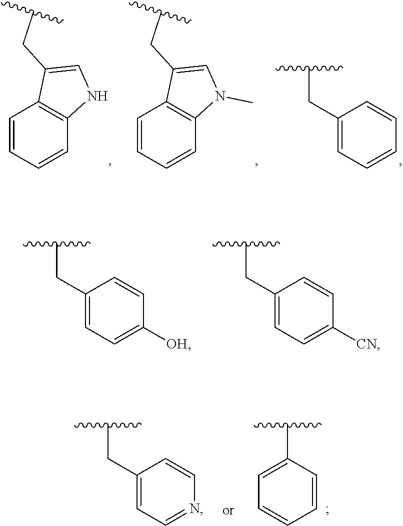

R¹³ is H or —CH₃; and
R¹⁴ is

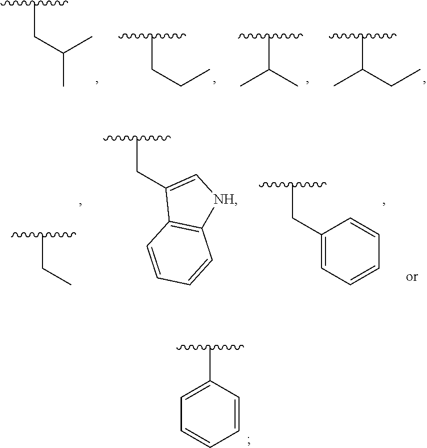

Embodiment 7. The compound of embodiment 6, or a pharmaceutically acceptable salt thereof, wherein:
R¹⁴ is

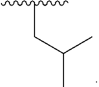 [SMILES: *CC(C)C]

Embodiment 8. The compound of embodiment 6 or 7, or a pharmaceutically acceptable salt thereof, wherein:
R¹² is

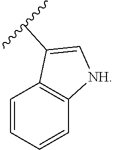 [SMILES: *c1c[nH]c2ccccc12]

Embodiment 9. The compound of embodiment 6, 7, or 8, or a pharmaceutically acceptable salt thereof, wherein:
R¹¹ is H or —CH-₃;
R¹² is

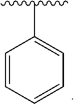 [SMILES: *c1ccccc1]

Embodiment 10 The compound of embodiment 1, or a pharmaceutically acceptable salt thereof, wherein:
X⁶ is phenylalanine (Phe), 3-fluorophenylalanine (3-F-Phe), biphenylalanine (Bip), or cyclohexylalanine (Cha);
X⁷ is glycine (Gly) or aza-glycine (aza-Gly); and
X⁸ is leucine (Leu) or norvaline (Nva).

Embodiment 11. The compound of embodiment 1, or a pharmaceutically acceptable salt thereof, wherein:
X⁶ is phenylalanine (Phe) or cyclohexylalanine (Cha);
X⁷ is aza-glycine (aza-Gly); and
X⁸ is leucine (Leu).

Embodiment 12. The compound of embodiment 1, or a pharmaceutically acceptable salt thereof, wherein:
X⁶ is

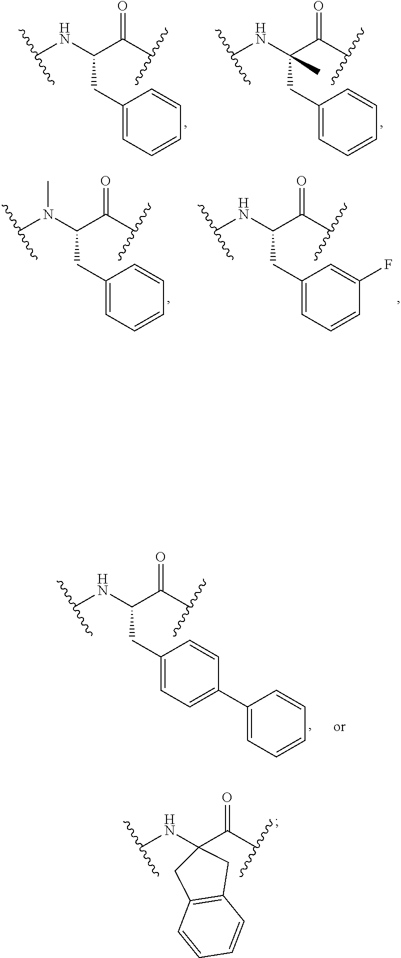

X⁷ is or

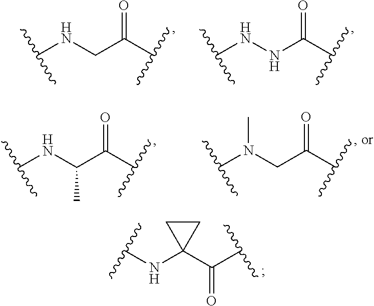

X⁸ is

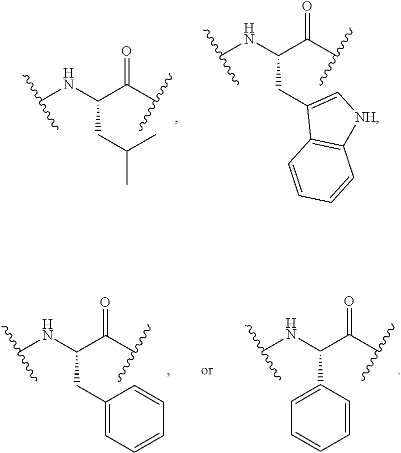

Embodiment 13. The compound of embodiment 12, or a pharmaceutically acceptable salt thereof, wherein:
X⁶ is

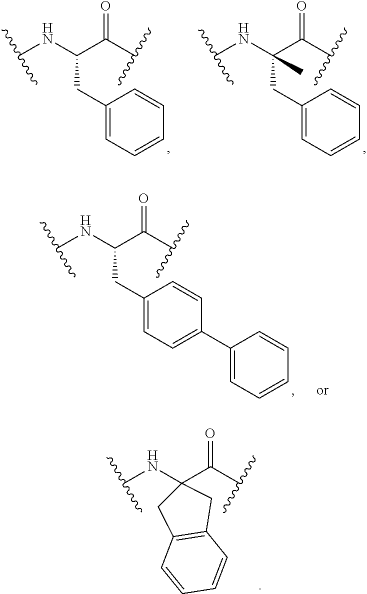

Embodiment 14 The compound of embodiment 12 or 13, or a pharmaceutically acceptable salt thereof, wherein:
X⁷ is

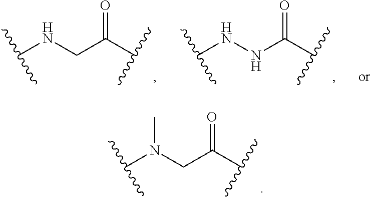

Embodiment 15. The compound of embodiment 12, 13, or 14, or a pharmaceutically acceptable salt thereof, wherein:
X⁸ is

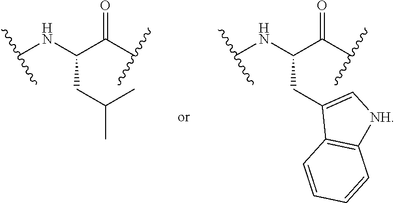

Embodiment 16. The compound of embodiment 1, or a pharmaceutically acceptable salt thereof, wherein:
X⁶ is

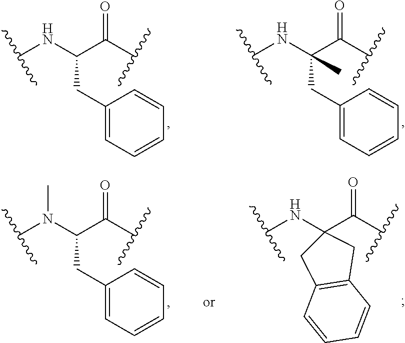

X⁷—X⁸— is

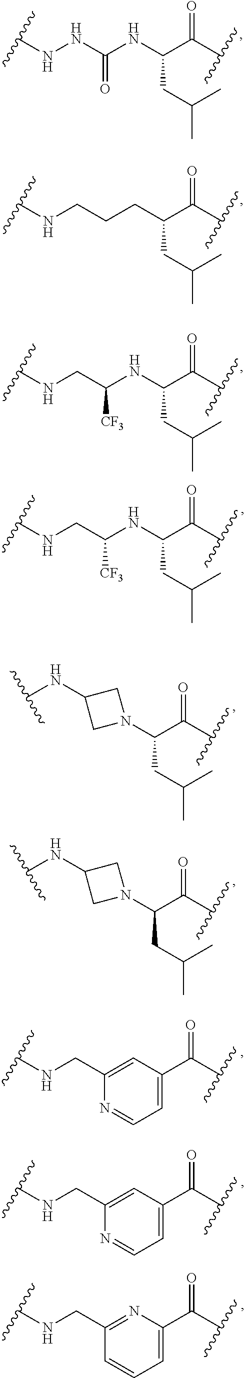

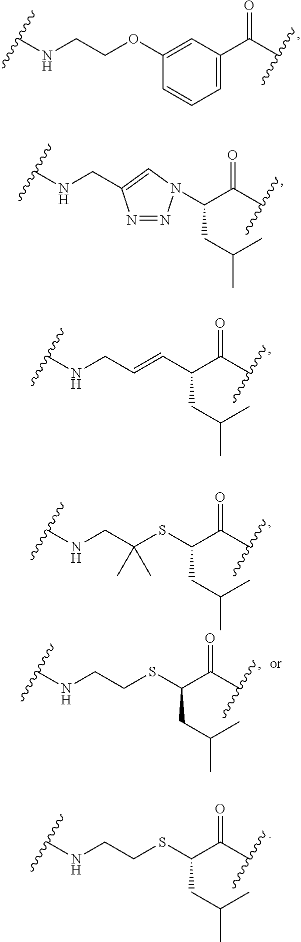

Embodiment 17. The compound of embodiment 1, or a pharmaceutically acceptable salt thereof, wherein:
—X⁶—X⁷—X⁸— is

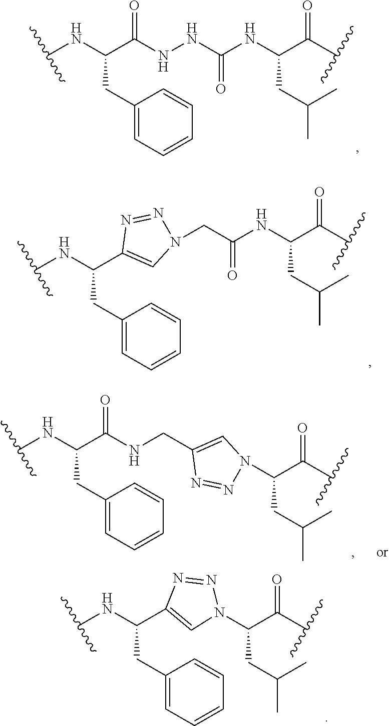

Embodiment 18. The compound of embodiment 1, or a pharmaceutically acceptable salt thereof, wherein:

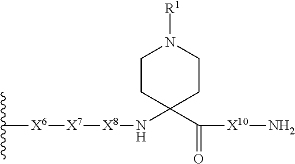

is

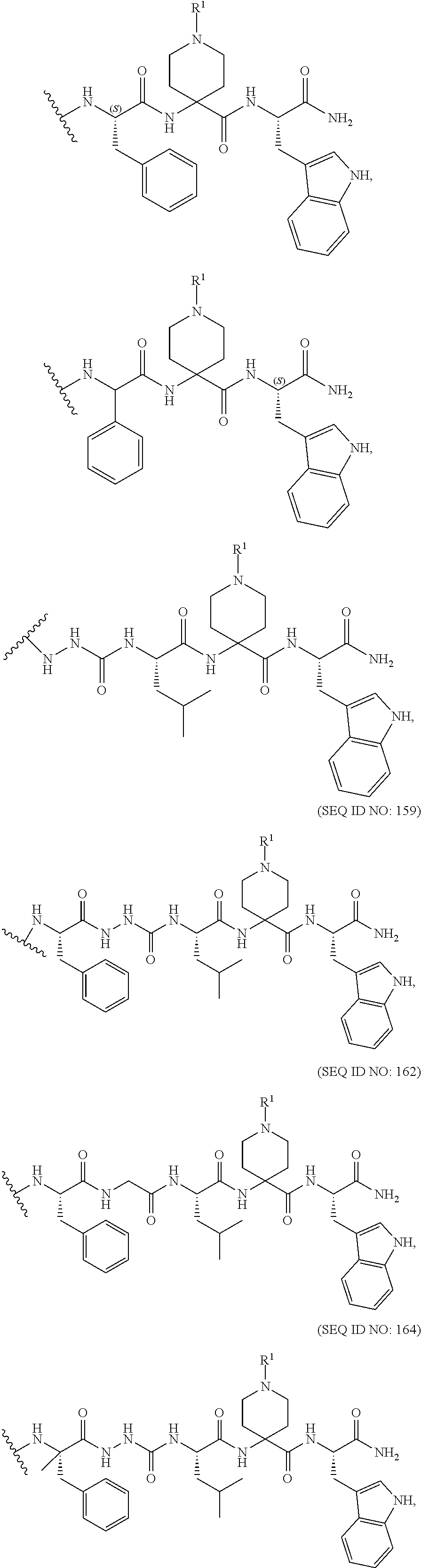

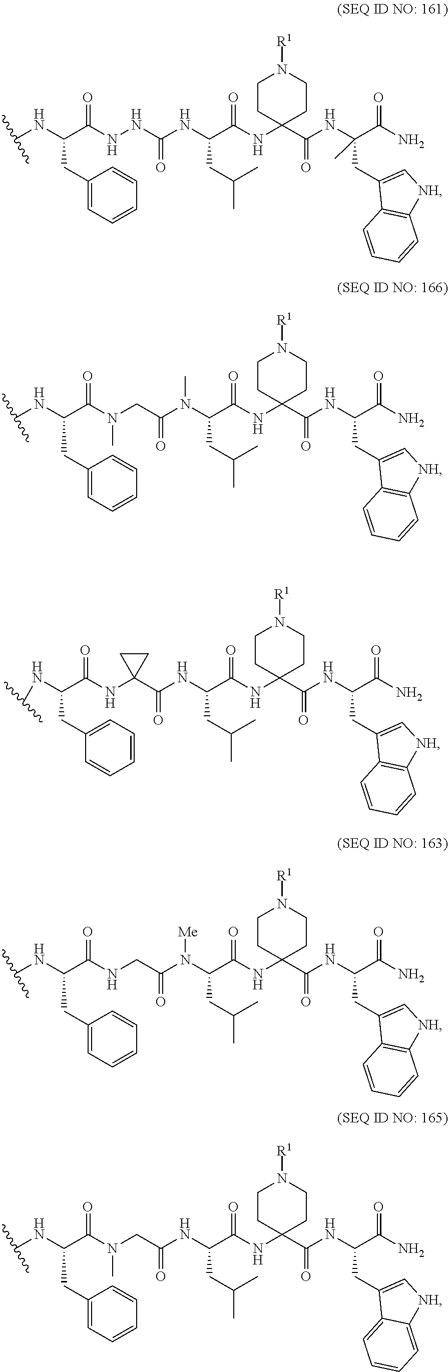

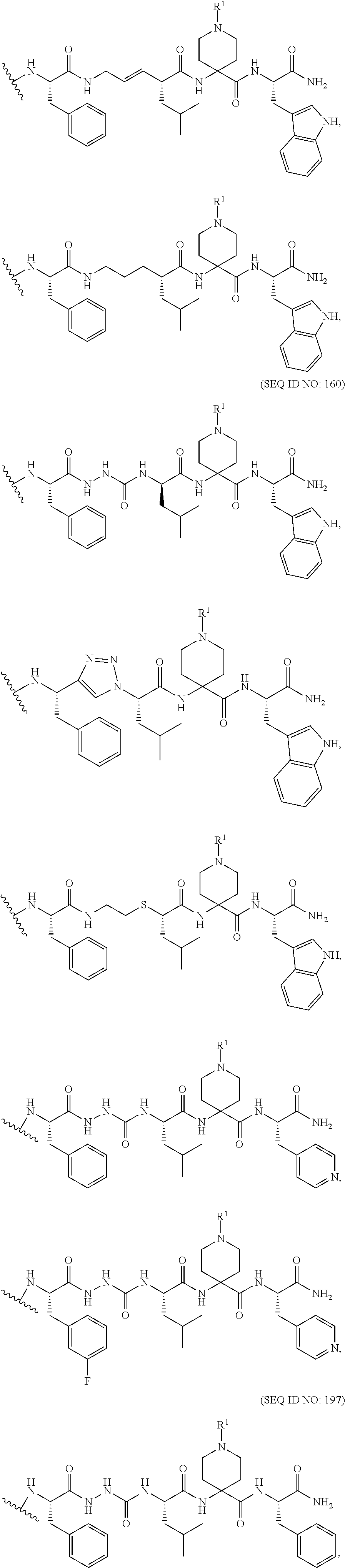

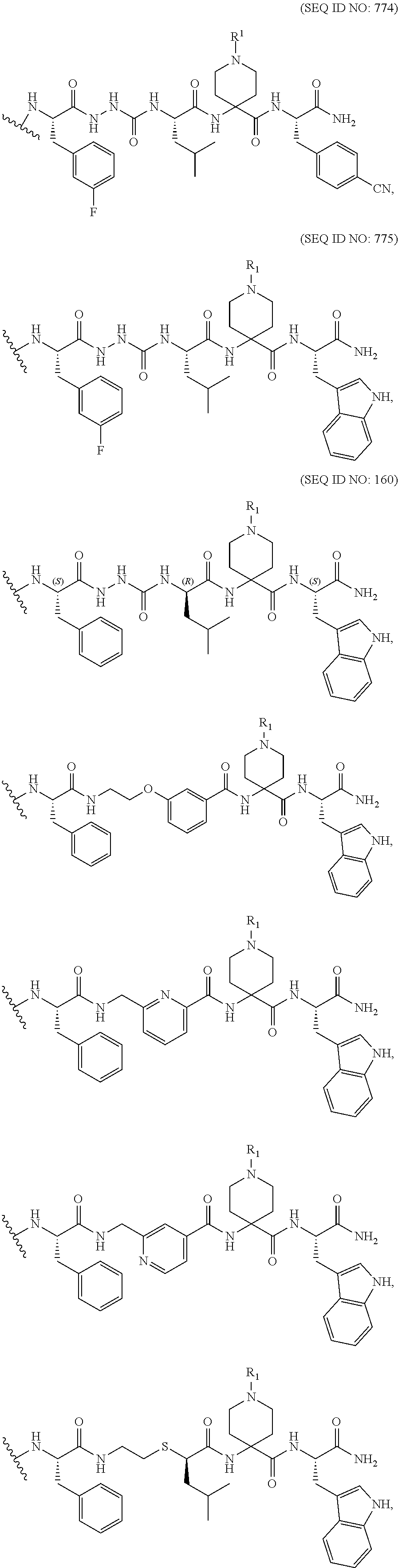

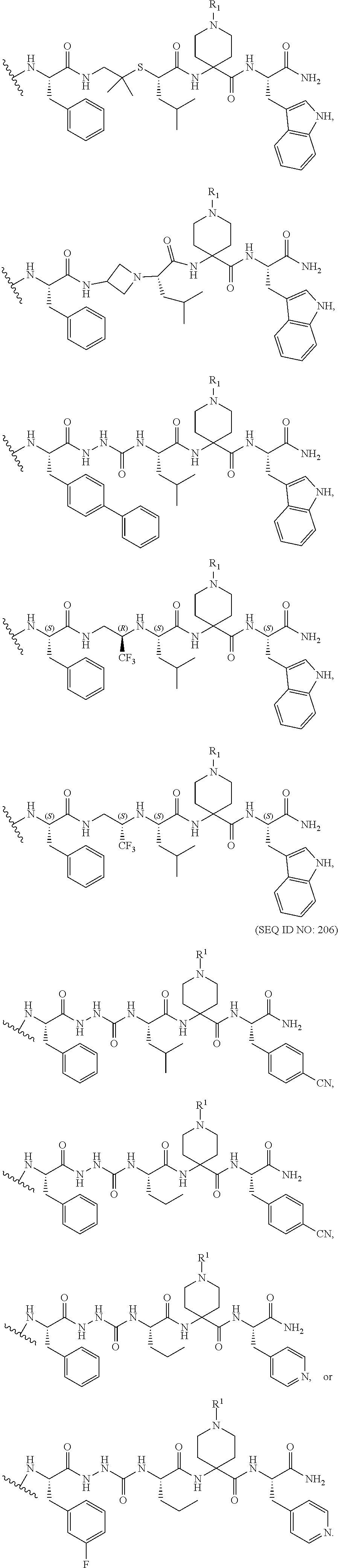

Embodiment 19. The compound of any one of embodiments 1-4 or 10-18, or a pharmaceutically acceptable salt thereof, wherein: X¹⁰ is tryptophan (Trp), 1-methyltryptophan (1MT), tyrosine (Tyr), phenylalanine (Phe), 4-cyano phenylalanine (Phe(4-CN)), 3-(4-pyridyl)alanine (4-Pal), or leucine (Leu).

Embodiment 20. The compound of any one of embodiments 1-4 or 10-18, or a pharmaceutically acceptable salt thereof, wherein: X¹⁰ is tryptophan (Trp), tyrosine (Tyr), phenylalanine (Phe), or 4-cyano phenylalanine (Phe(4-CN)).

Embodiment 21. The compound of any one of embodiments 1-20, or a pharmaceutically acceptable salt thereof, wherein:
R³ is H or CH₃;
R⁴ is H, —CH₃, or R².

Embodiment 22. The compound of any one of embodiments 1-21, or a, pharmaceutically acceptable salt thereof, wherein:
R¹ is

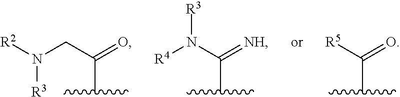

Embodiment 23. The compound of any one of embodiments 1-21, or a pharmaceutically acceptable salt thereof, wherein R¹ is H.

Embodiment 24. The compound of any one of embodiments 1-21, or a pharmaceutically acceptable salt thereof, wherein:
R¹ is

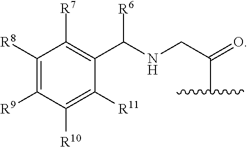

Embodiment 25. The compound of any one of embodiments 1-21, or a pharmaceutically acceptable salt thereof, wherein: R¹ is

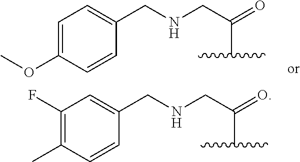

Embodiment 26. The compound of any one of embodiments 1-21, or a pharmaceutically acceptable salt thereof, wherein:
R¹ is

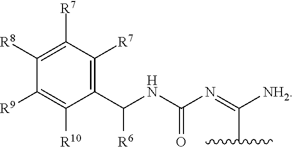

Embodiment 27. The compound of any one of embodiments 1-26, or a pharmaceutically acceptable salt thereof, wherein:
R² is

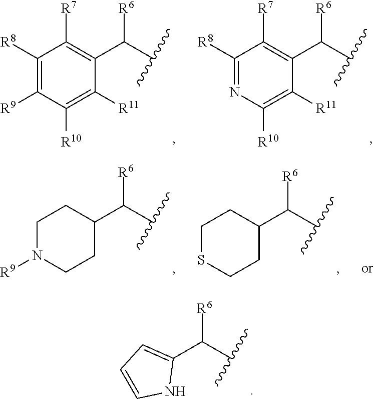

Embodiment 28. The compound of any one of embodiments 1-26, or a pharmaceutically acceptable salt thereof, wherein:
R² is

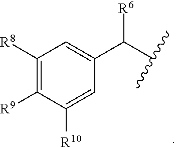

Embodiment 29. The compound of any one of embodiments 1-26, or a pharmaceutically acceptable salt thereof, wherein: R² is C₁-C₆ alkyl, wherein C₁-C₆ alkyl is optionally substituted with R⁷.

Embodiment 30. The compound of any one of embodiments 1-29, or a pharmaceutically acceptable salt thereof, wherein:
R⁵ is

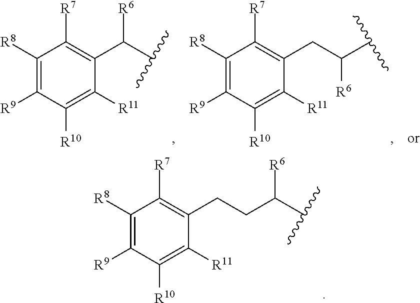

Embodiment 31 The compound of any one of embodiments 1-30, or a pharmaceutically acceptable salt thereof, wherein:
R⁷, R⁸, R⁹, R¹⁰, and R¹¹ are independently selected from H, F, Cl, Br, I, —OH, —OCH₃, —OCH₂CH₃, —NH₂, —NHCH₃, —N(CH₃)₂, —CN, —CO₂H, —CO₂CH₃, —CO₂CH₂CH₃, —CH₃, —CH₂CH₃, —CH(CH₃)₂, —(CH₃)₃, —CF₃, —CH₂F, —CH₂F, or cyclopropyl.

Embodiment 32. The compound of any one of embodiments 1-21, or a pharmaceutically acceptable salt thereof, wherein:
R¹ is

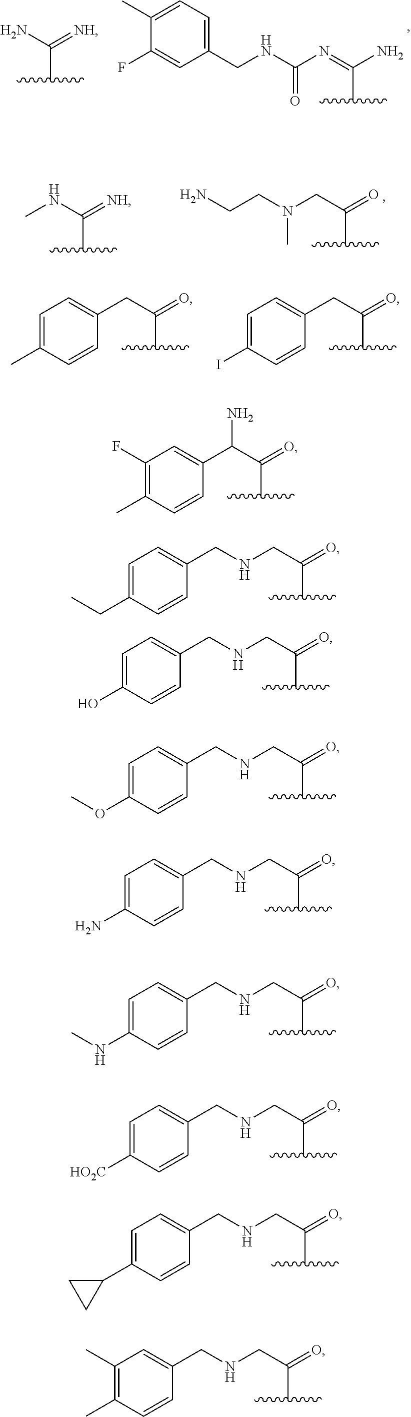

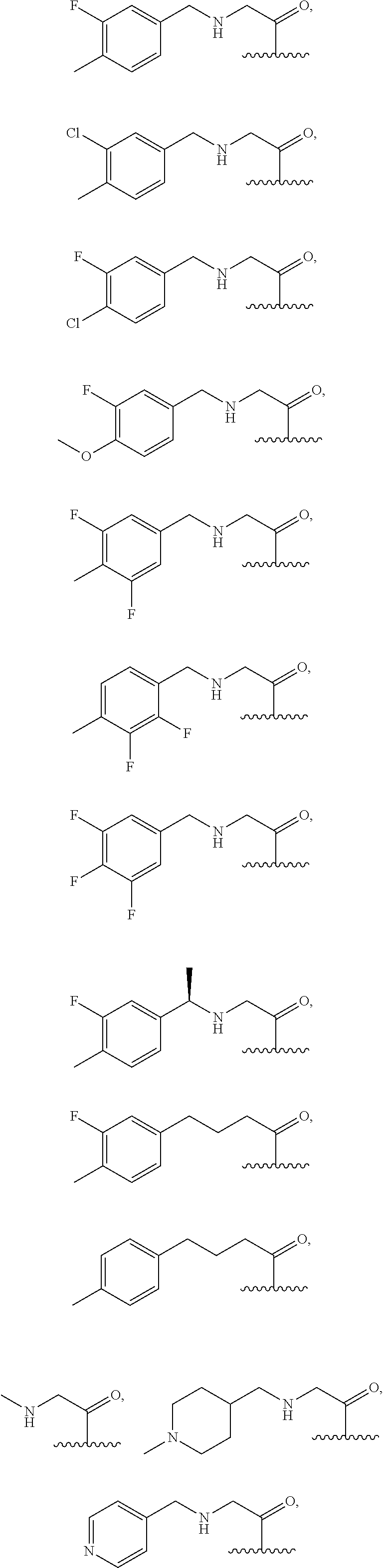

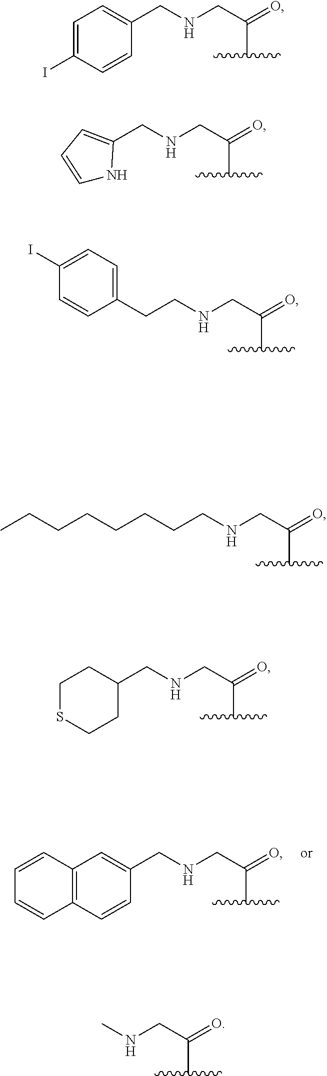

Embodiment 33, The compound of any one of embodiments 1-21, or a pharmaceutically acceptable salt thereof, wherein:
R¹ is

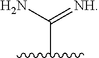 [SMILES: *C(=N)N]

Embodiment 34. The compound of any one of embodiments 1-21, or a pharmaceutically acceptable salt thereof, wherein: R¹ is

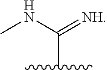 [SMILES: *C(=N)NC]

Embodiment 35. The compound of any one of embodiments 1-21, or a pharmaceutically acceptable salt thereof, wherein:
R¹ is

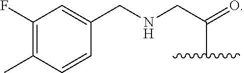 [SMILES: *C(=O)CNCc1ccc(C)c(F)c1]

Embodiment 36. The compound of any one of embodiments 1-21, or a pharmaceutically acceptable salt thereof, wherein:
R¹ is

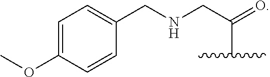 [SMILES: *C(=O)CNCc1ccc(OC)cc1]

Embodiment 37. The compound of any one of embodiments 1-21, or a pharmaceutically acceptable salt thereof, wherein.
X¹ is absent, Tyr, Asp, Lys, 3-Pal, Sar, or Phe; and
X² is absent, Asn, Gln, Asp, Glu, Ser, His, Ala, Sar, Pro, Hyp, Aze, Tic, Phe, or 4-Pal.

Embodiment 38. The compound of any one of embodiments 1-21, or a pharmaceutically acceptable salt thereof, wherein
X¹ is absent, D-Tyr, Asp, Lys, D-3-Pal, Sar, or Phe; and
X² is absent, D-Asn, Gln, Asp, Glu, Ser, His, Ala, Sar, Pro, Hyp, Aze, Tic, Phe, or 4-Pal.

Embodiment 39. The compound of any one of embodiments 1-38, or a pharmaceutically acceptable salt thereof, wherein
X³ is absent, Trp, Ser, Ile, Phe, 4-Pal, Lys, Asn, Gln, Asp, Glu, Arg, Arg(Me), AGBA, AGBA(Me), Harg, Harg(Me), Cit, Cit(Me), canavanine, methyl-canavanine, Gly, Ala, Sar, Tyr, Cha, β-Nal, Hyp, Thr, Bip, Bpa, or H-Ala(9-Anth)-OH;
X⁴ is absent, Asn, Gln, Asp, Glu, Trp, Gly, Ala, or Sar; and
X⁵ is absent, Ser, Thr, Asn, Gln, Asp, Glu, Gly, Ala or Sar.

Embodiment 40. The compound of any one of embodiments 1-38, or a pharmaceutically acceptable salt thereof, wherein:
X³ is absent, Trp, Ser, lie, Phe, 4-Pal, Lys, Asn, Gln, Asp, Glu, Arg, Arg(Me), AGBA, AGBA(Me), Harg, Harg(Me), Cit, Cit(Me), canavanine, methyl-canavanine, Gly, Ala, Sar, Tyr, Cha, β-Nal, Hyp, Thr, Bip, Bpa, or AAP;
X⁴ is absent, -Asn, Gln, Asp, Glu, Trp, Gly, Ala, or Sar; and
X⁵ is absent, Thr, Ser or Ala.

Embodiment 41. The compound of any one of embodiments 1-38, or a pharmaceutically acceptable salt thereof, wherein:
X¹ is absent, Tyr, Asp, Lys, 3-Pal, Sar, or Phe;
X² is absent, Asn, Gin, Asp, Glu, Ser, His, Ala, Sar, Pro, Hyp, Aze, Tic, Phe, or 4-Pal;
X³ is absent, Trp, Ser, Ile, Phe, 4-Pal, Lys, Asn, Gin, Asp, Glu, Arg, Arg(Me), Gly, Ala, Sar, Tyr, Cha, β-Nal, Fyp, Thr, Bip, Bpa, or AAP;
X⁴ is absent, Asn, Gln, Asp, Glu, Trp, Gly, Ala, or Sar; and
X⁵ is absent, Thr, Ser, Gly, or Ala.

Embodiment 42. The compound of any one of embodiments 1-38, or a pharmaceutically acceptable salt thereof, wherein

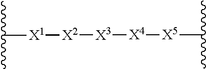

is absent,

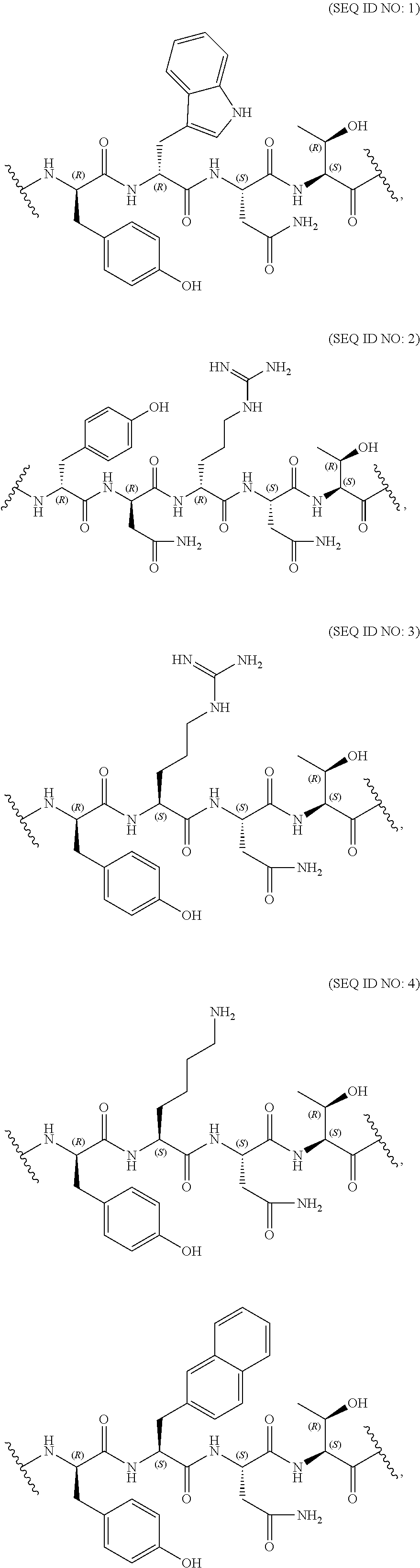

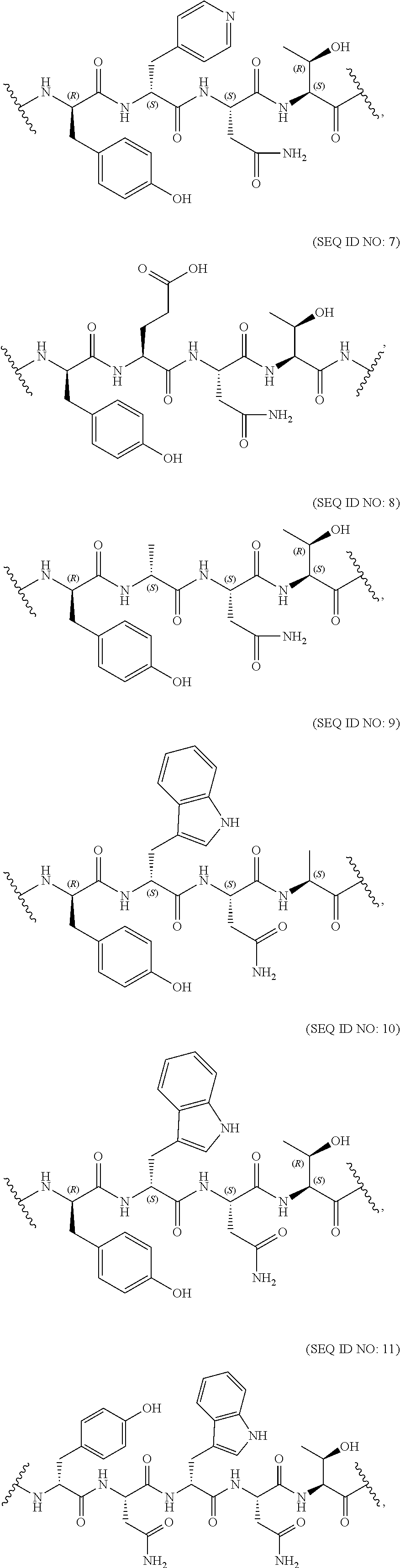

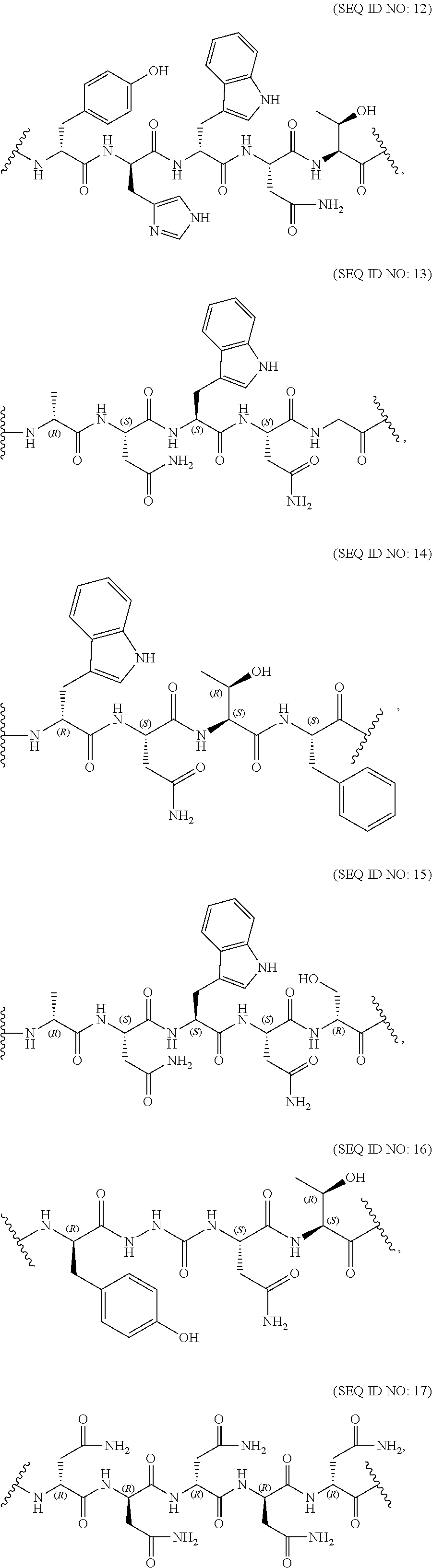

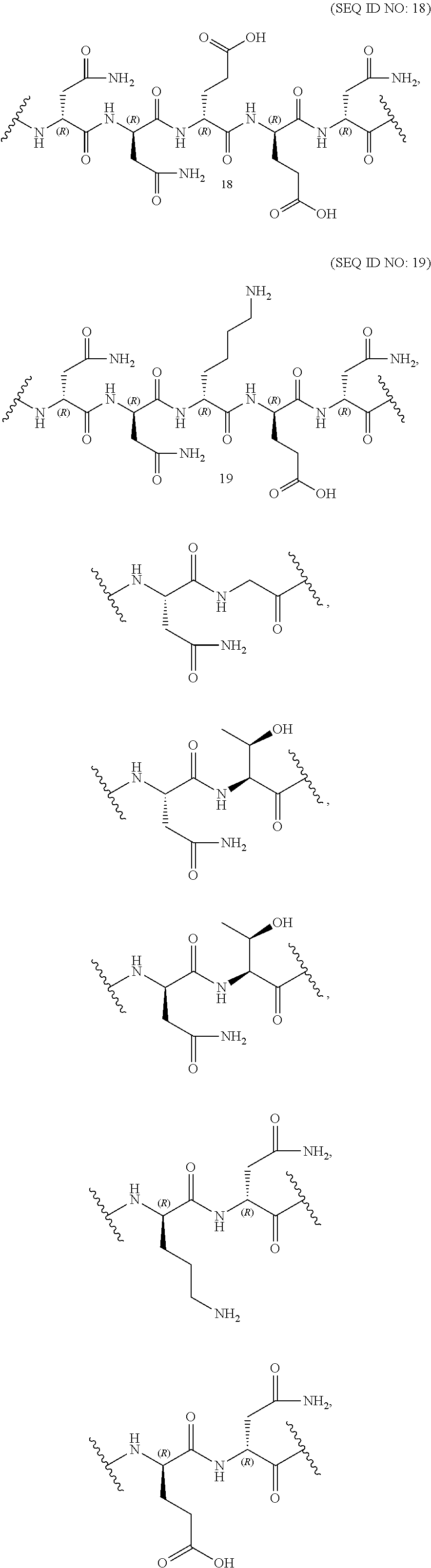

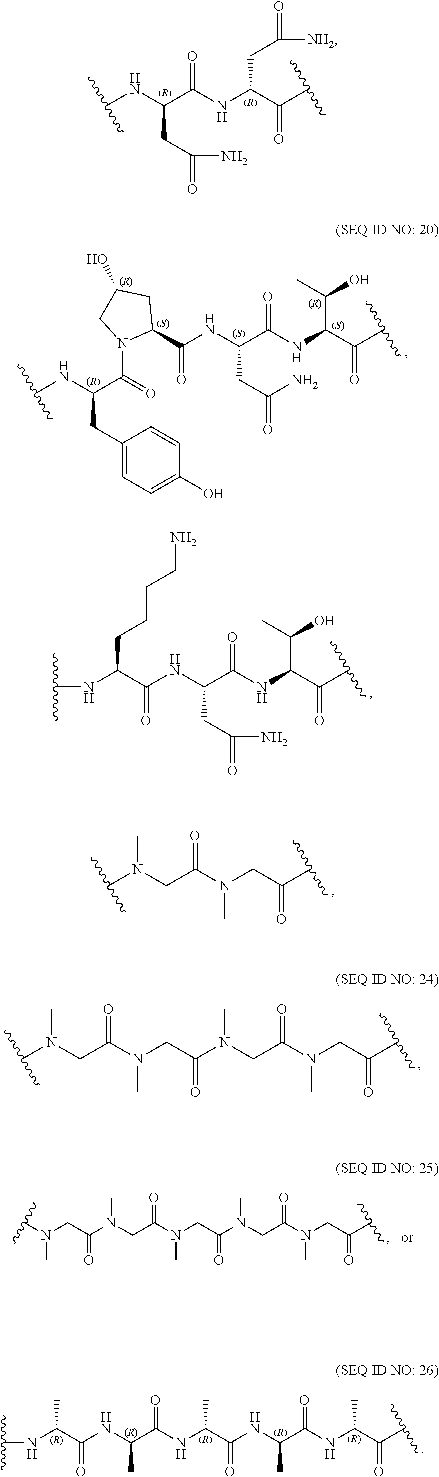

Embodiment 43. The compound of any one of embodiments 1-38, or a pharmaceutically acceptable salt thereof, wherein

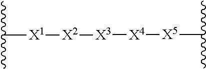

is absent, Asn-, -Lys-, -2-Pal-, -Thr-, -Trp-, -Asn-Asn-, -Asn-Gly-, -Asn-Thr-, -Glu-Asn-, -Glu-Thr-, -Lys-Asn-,-Lys(DOTA)-Asn-, -Lys-Thr-, -Lys(DOTA)-Thr-, -Lys-Glu-, -Lys(DOTA)-Glu-, -Sar-Sar-, -AAP-Asn-Thr, -Asn-Phe-Thr-, -Glu-Asn-Thr-, -D-Glu-Asn-Thr-, -bGlu-Asn-Thr-, -Gly-Tyr-Ahx-, -Lys-Asn-Thr-, -D-β-Nal-Asn-Thr-, -D-4Pal-Asn-Thr-, -Thr-Asn-Arg-, -Trp-Asn-Thr-, -D-Trp-Asn-Thr-, -D-Tyr-Asn-Thr-, -Lys-Asn-Thr-, -Lys(DOTA)-Asn-Thr-, -Gly-Tyr-β-Nal-Ahx-, -Lys-Trp-Asn-Thr- (SEQ ID NO: 28), -Phe(4-I)-Trp-Asn-Thr- (SEQ ID NO: 29), -D-Phe(4-I)-D-Trp-Asn-Thr- (SEQ ID NO: 30), -Sar-Sar-Sar-Sar- (SEQ ID NO: 24), D-Trp-Asn-Thr-Phe-(SEQ ID NO: 14), -D-Tyr-AAP-Asn-Thr-, -D-Tyr-Arg-Asn-Thr- (SEQ ID NO: 3), -D-Tyr-D-Ala-Asn-Thr-(SEQ ID NO: 8), -Tyr-AzaGly-Asn-Thr- (SEQ ID NO: 32), -D-Tyr-AzaGly-Asn-Thr-(SEQ ID NO: 16), -D-Tyr-Bip-Asn-Thr-, -D-Tyr-Bpa-Asn-Thr-, -D-Tyr-Glu-Asn-Thr-(SEQ ID NO: 7), -D-Tyr-Hyp-Asn-Thr-, -D-Tyr-D-Hyp-Asn-Thr-, -Tyr-Hyp-Asn-Thr-D-Tyr-Lys-Asn-Thr- (SEQ ID NO: 4), -D-Tyr-Lys(DOTA)-Asn-Thr- (SEQ ID NO: 38), 1HO—(CH₂CH₂)₂—CH₂C(═O)-D-Tyr-Lys(DOTA)-Asn-Thr- (SEQ ID NO: 21), -Tyr-D-Lys-Asn-Thr-, -Tyr-D-Lys(DOTA)-Asn-Thr-(SEQ ID NO: 39), -D-Tyr-D-Lys-Asn-Thr-, -D-Tyr-D-Lys(DOTA)-Asn-Thr-(SEQ ID NO: 40), -Tyr-β-Nal-Asn-Thr-, -D-Tyr-β-Nal-Asn-Thr-, -D-Tyr-D-3-Nal-Asn-Thr-, -Tyr-4Pal-Asn-Thr-, -D-Tyr-4Pal-Asn-Thr-, -D-Tyr-D-4Pal-Asn-Thr-, -D-Tyr-Phe(4-I)-Asn-Thr- (SEQ ID NO: 45), -Tyr-Pro-Asn-Thr- (SEQ ID NO: 46), -D-Tyr-Pro-Asn-Thr-(SEQ ID NO: 20), D-Tyr-Trp-Asn-Ala- (SEQ ID NO: 9), -D-Tyr-D-Trp-Asn-Ala-(SEQ ID NO: 47), -D-Tyr-Trp-Asn-Thr- (SEQ ID NO: 10), -D-Tyr-D-Trp-Asn-Thr- (SEQ ID NO: 1), -D-Ala-D-Ala-D-Ala-D-Ala-D-Ala- (SEQ ID NO: 26), D-Ala-Asn-Trp-Asn-Gly- (SEQ ID NO: 13), D-Ala-Asn-Trp-Asn-D-Ser (SEQ ID NO: 15), -D-Asn-D-Asn-D-Asn-D-Asn-D-Asn-(SEQ ID NO: 17), -D-Asn-D-Asn-D-Glu-D-Glu-D-Asn- (SEQ ID NO: 18), -D-Asn-D-Asn-D-Lys-D-Glu-D-Asn-(SEQ ID NO: 19), -γ-D-Glu-D-Tyr-Lys(DOTA)-Asn-Tin- (SEQ ID NO: 48) -Glu-D-Tyr-β-Nal-Asn-Thr- (SEQ ID NO: 49), -Gly-Tyr-D-Trp-Asn-Thr- (SEQ ID NO: 50), -Sar-Sar-Sar-Sar-Sar- (SEQ ID NO: 25), -Tyr-Asn-Trp-Asn-Ser- (SEQ ID NO: 51), -D-Tyr-D-Asn-D-Arg-Asn-Thr-(SEQ ID NO: 2), -D-Tyr-Asn-D-Trp-Asn-Thr (SEQ II) NO: 11), -Tyr-Glu-Asn-Thr-3-F-Phe- (SEQ ID NO: 52), -D-Tyr-D-His-D-Trp-Asn-Thr- (SEQ ID NO: 12), -Tyr-D-Trp-Asn-Thr-3-F-Phe- (SEQ ID NO: 53), -D-Tyr-D-Trp-Asn-Thr-3-F-Phe- (SEQ ID NO: 54), Palmitic Acid-,γGlu-Lys(DOTA)-Tyr-Asn-Trp-Asn-Ser- (SEQ ID NO: 846), or Ac-γGlu-Lys(DOTA)-D)-Ala-Asn-Trp-Asn-Gly-(SEQ ID NO: 23).

Embodiment 44. The compound of embodiment 1, or a pharmaceutically acceptable salt, thereof, wherein:

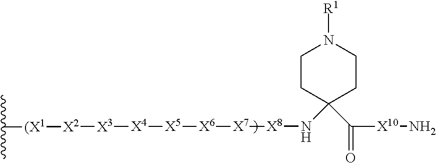

is

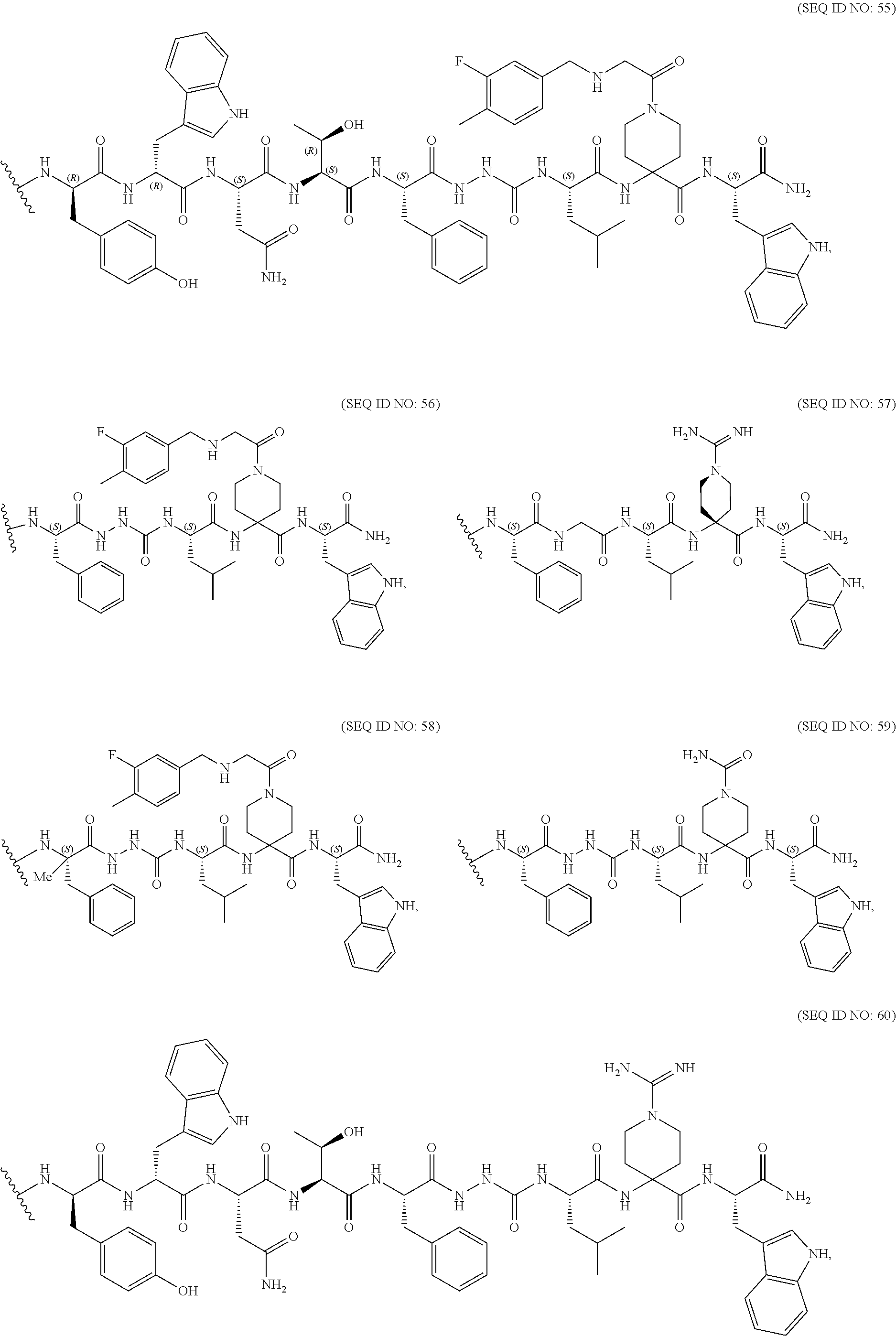

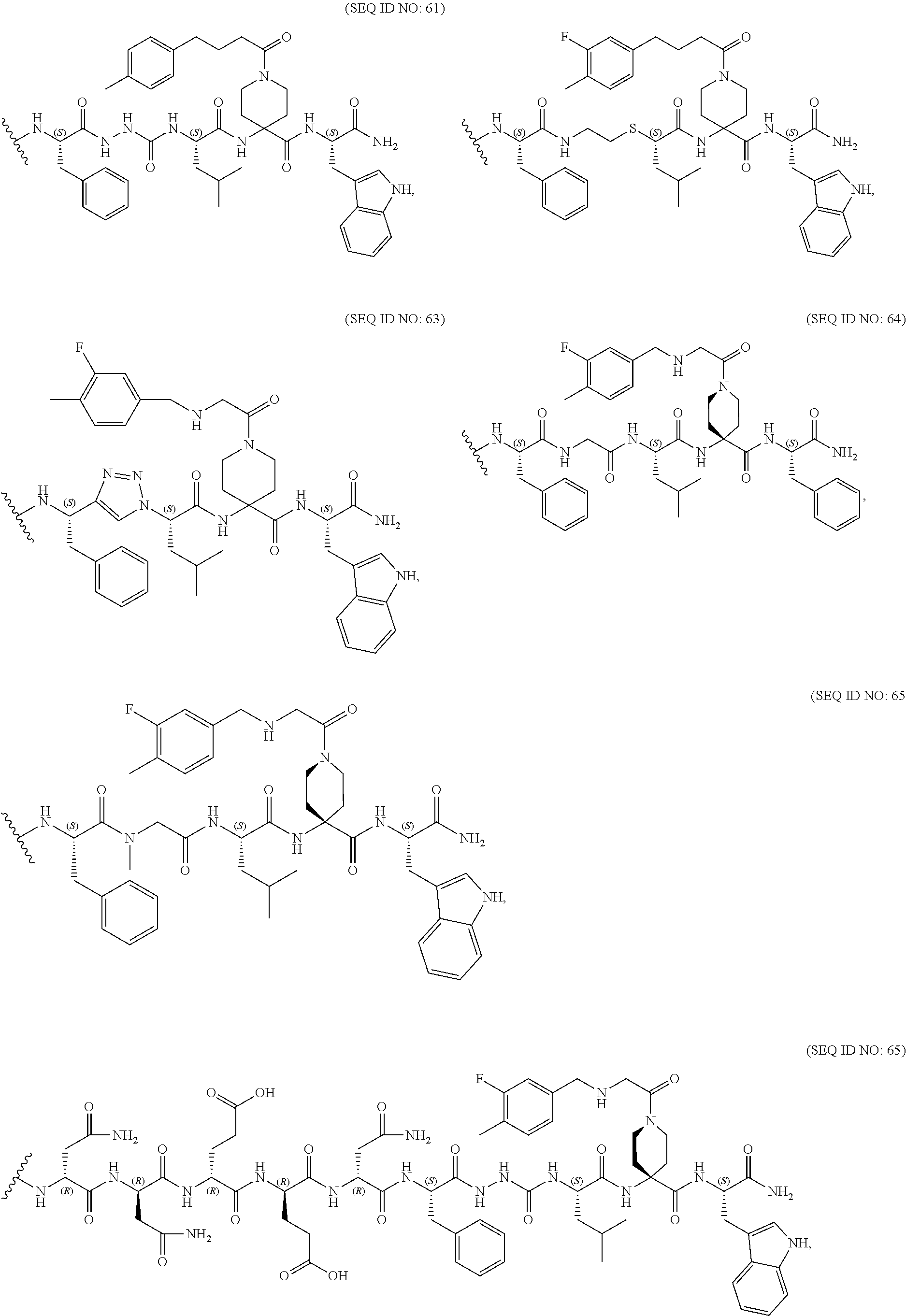

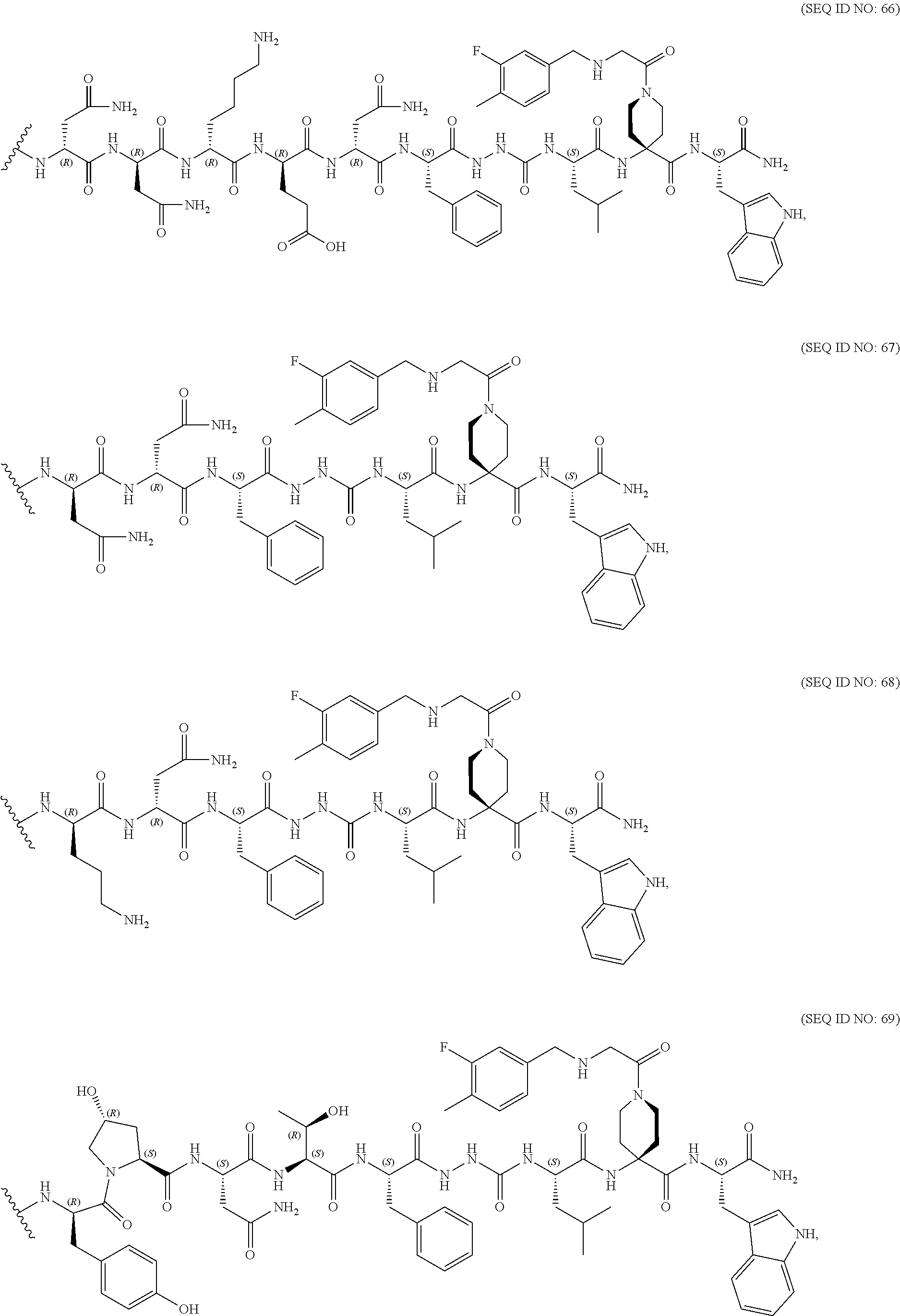

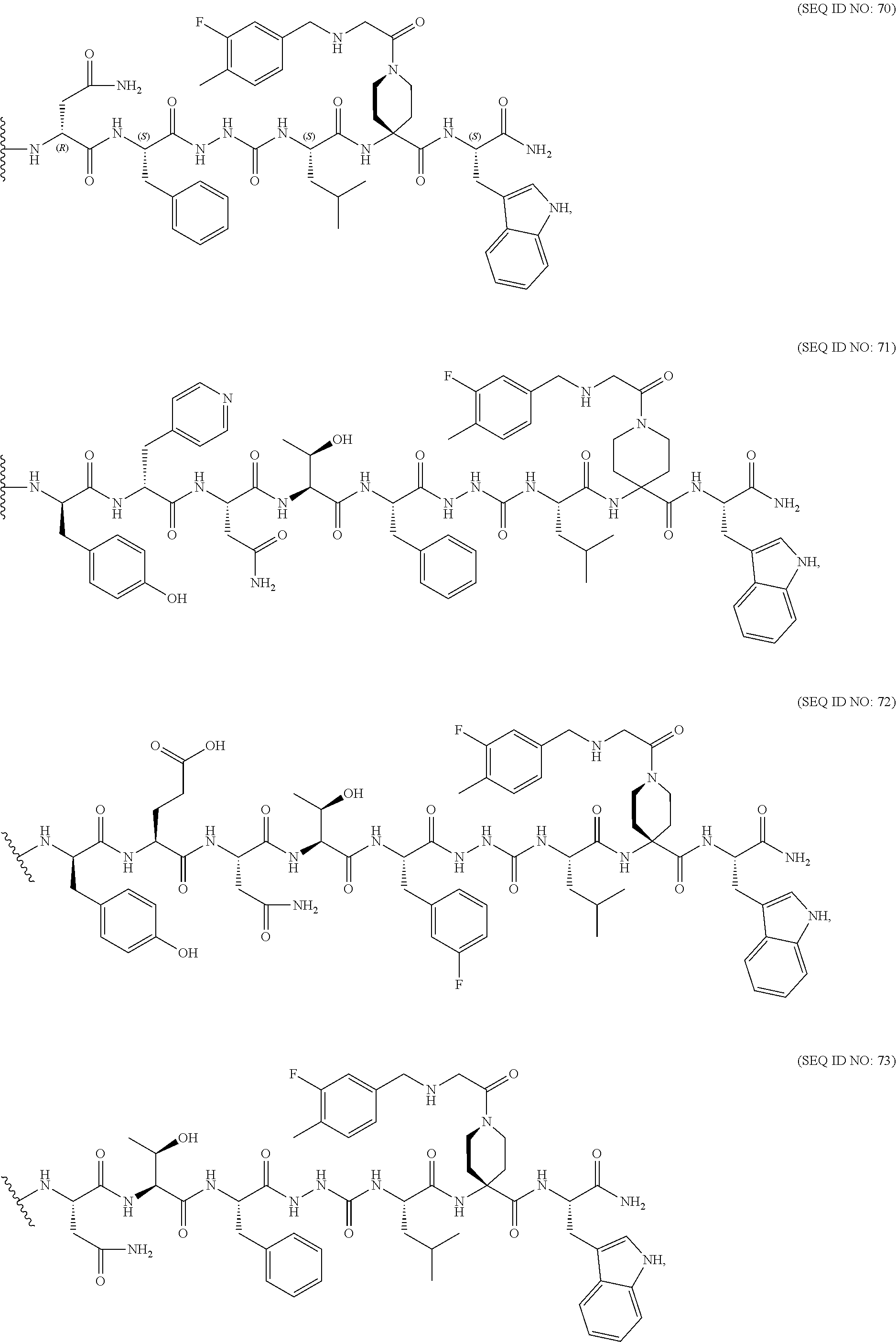

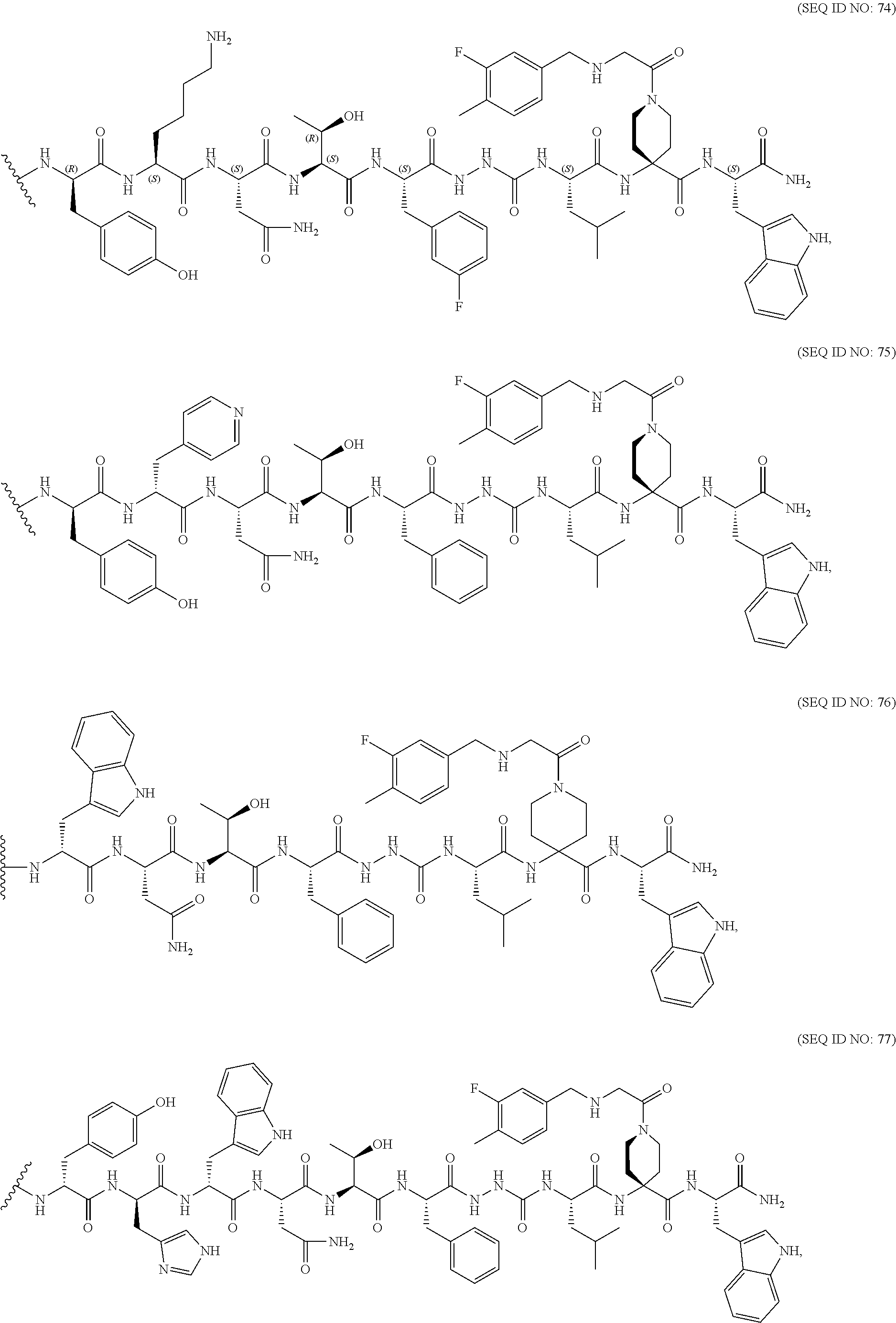

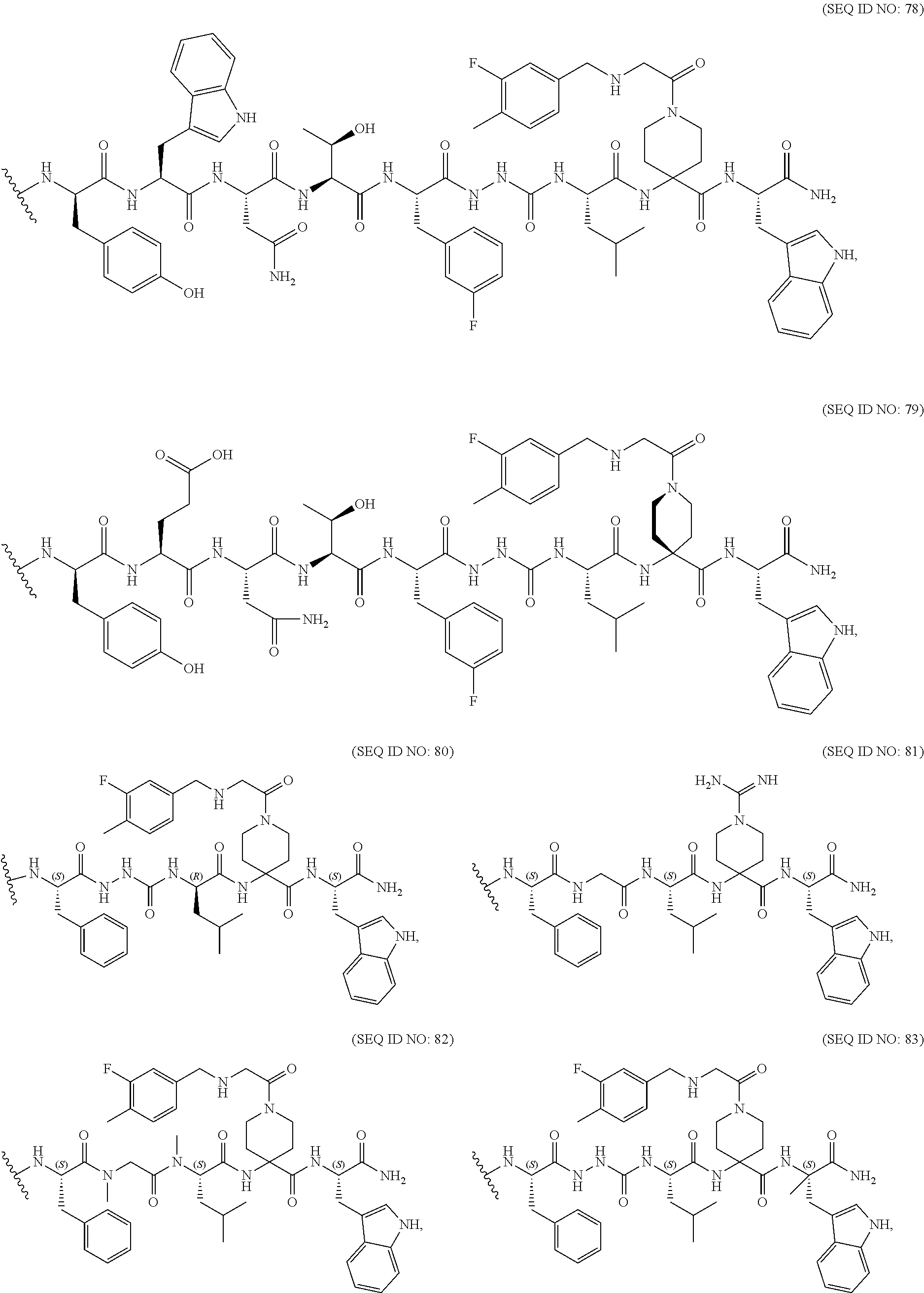

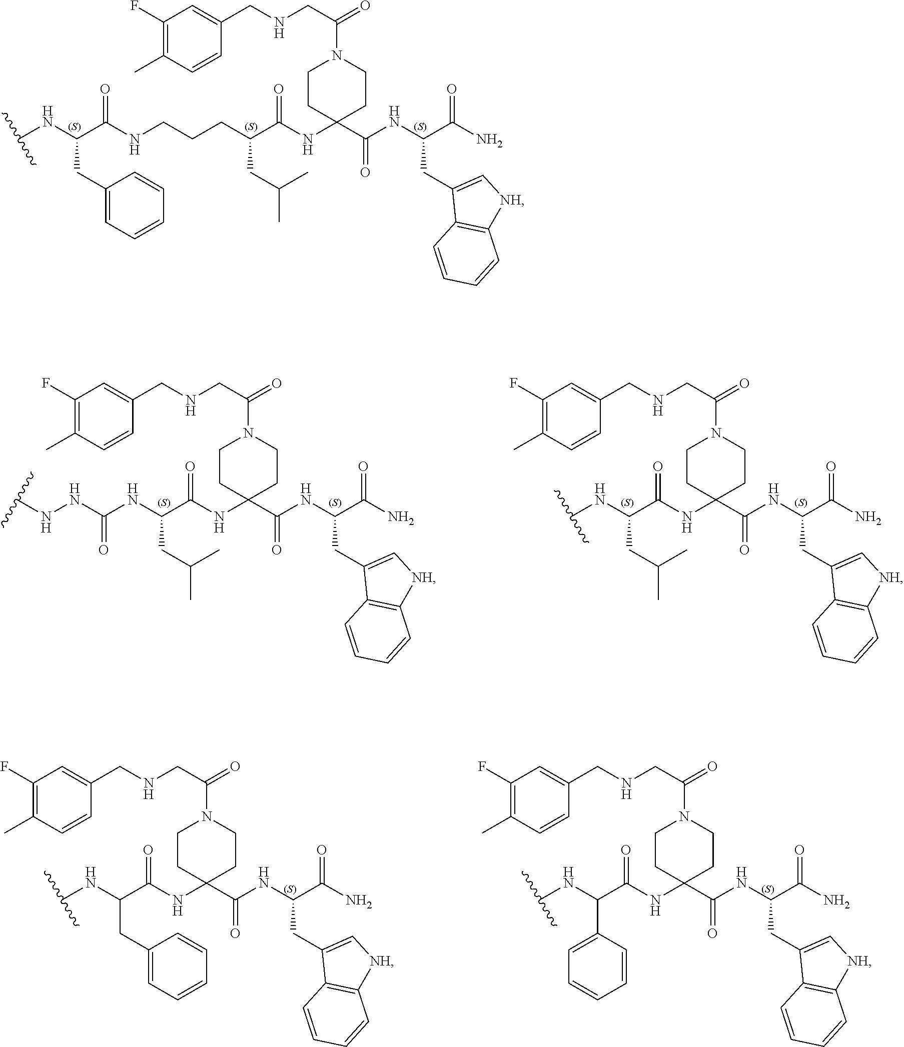

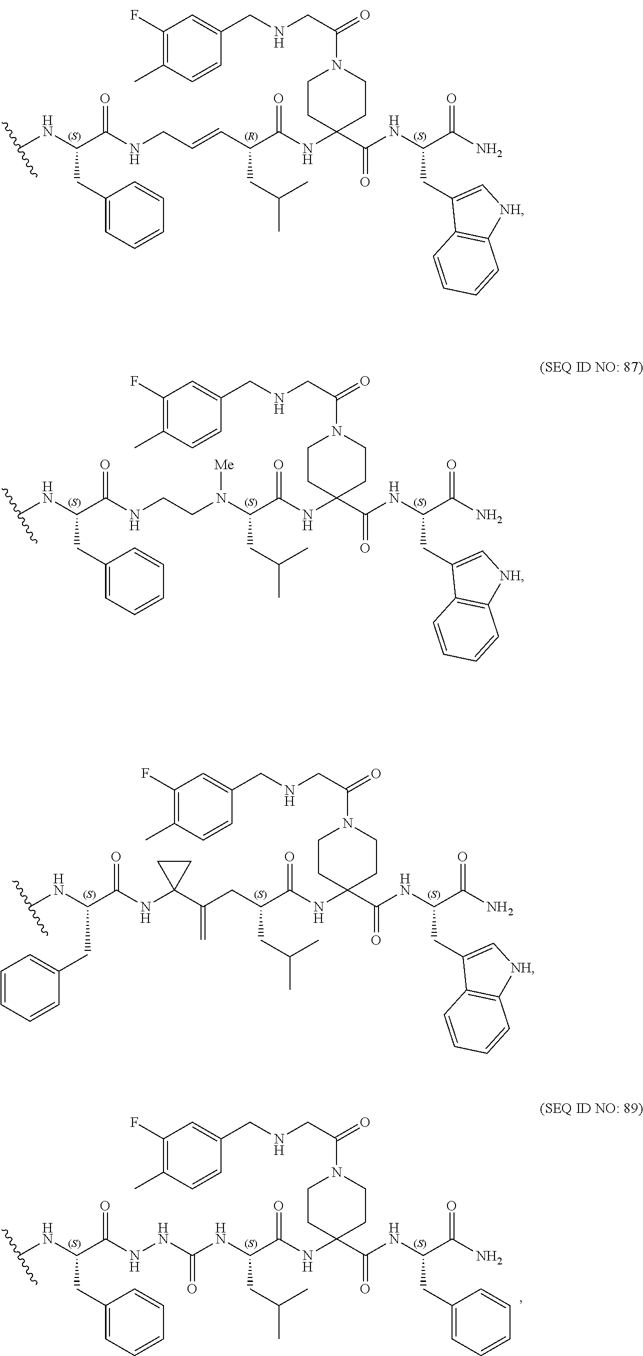

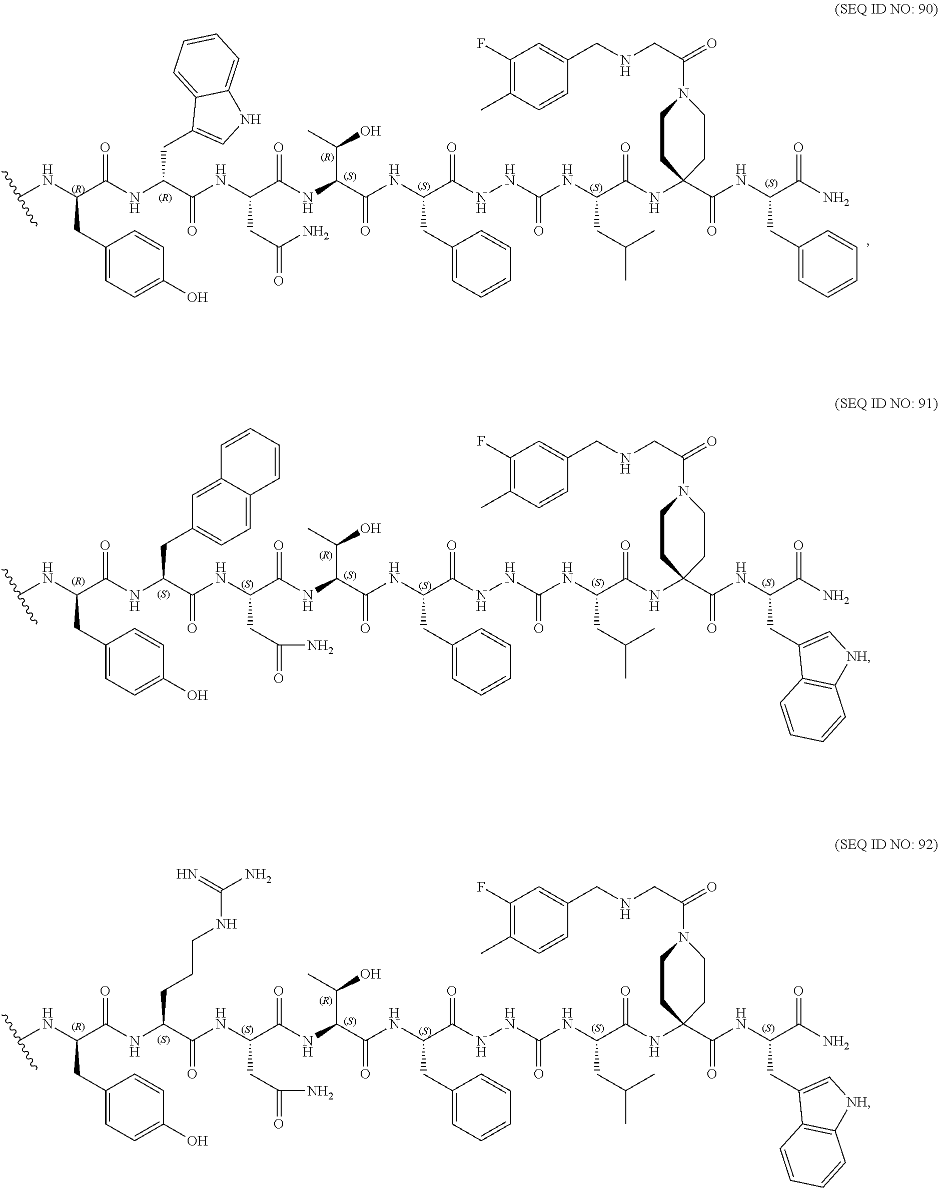

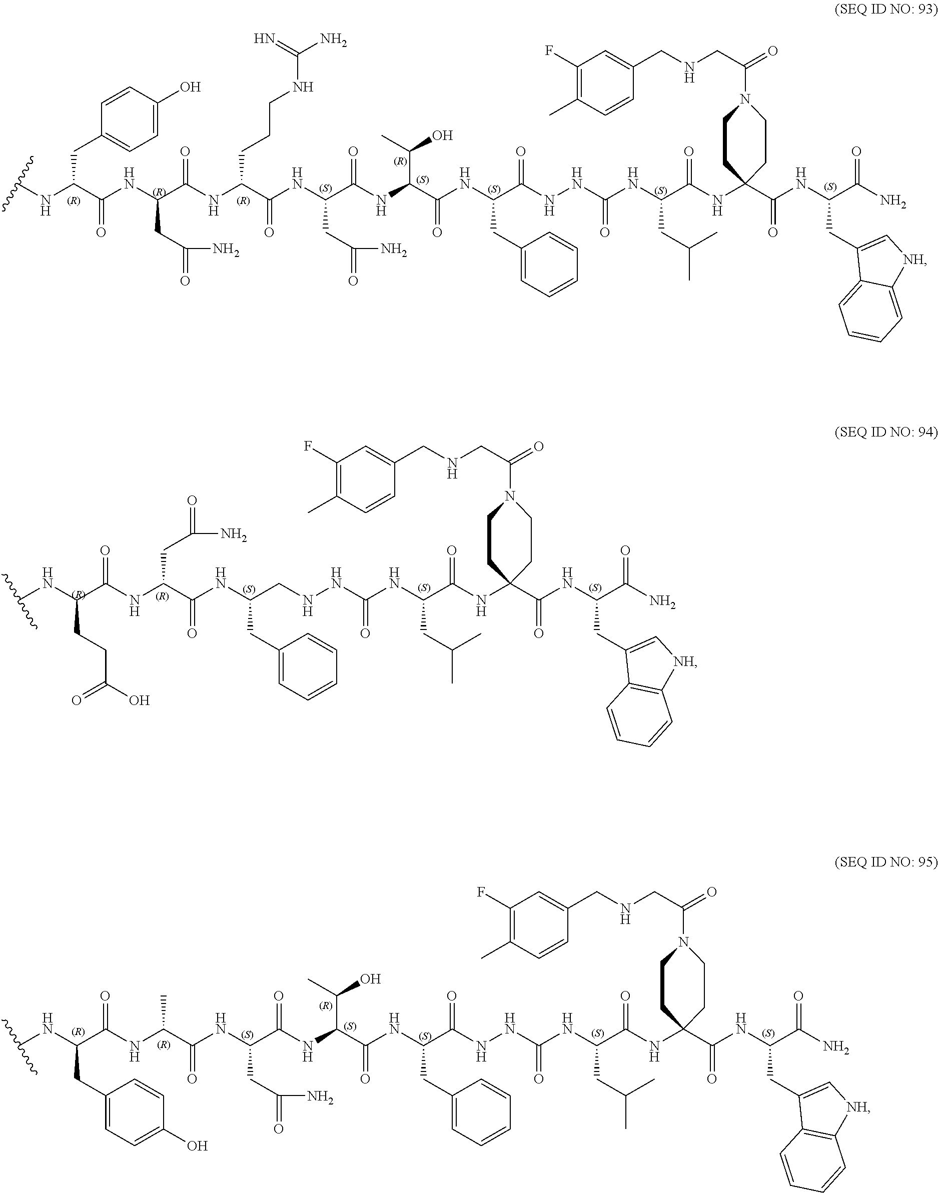

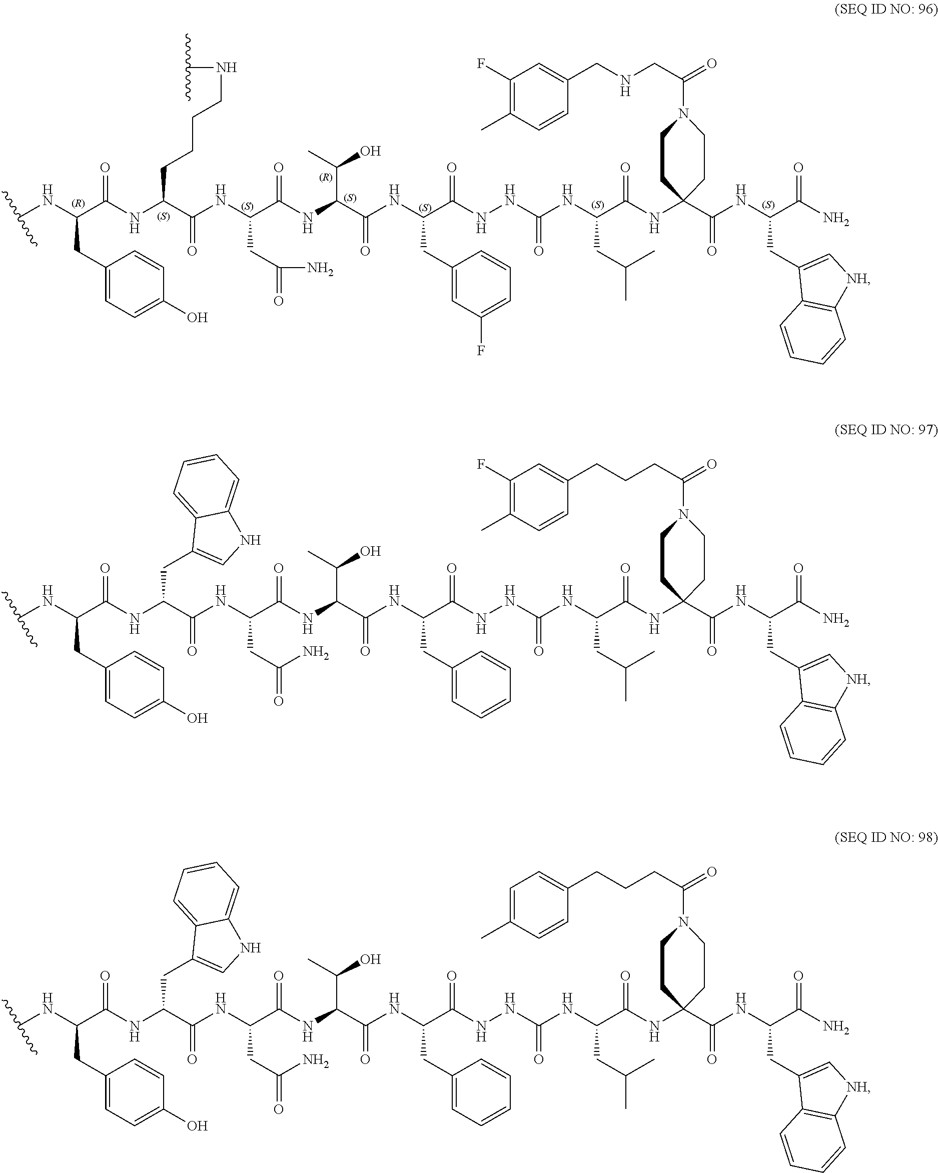

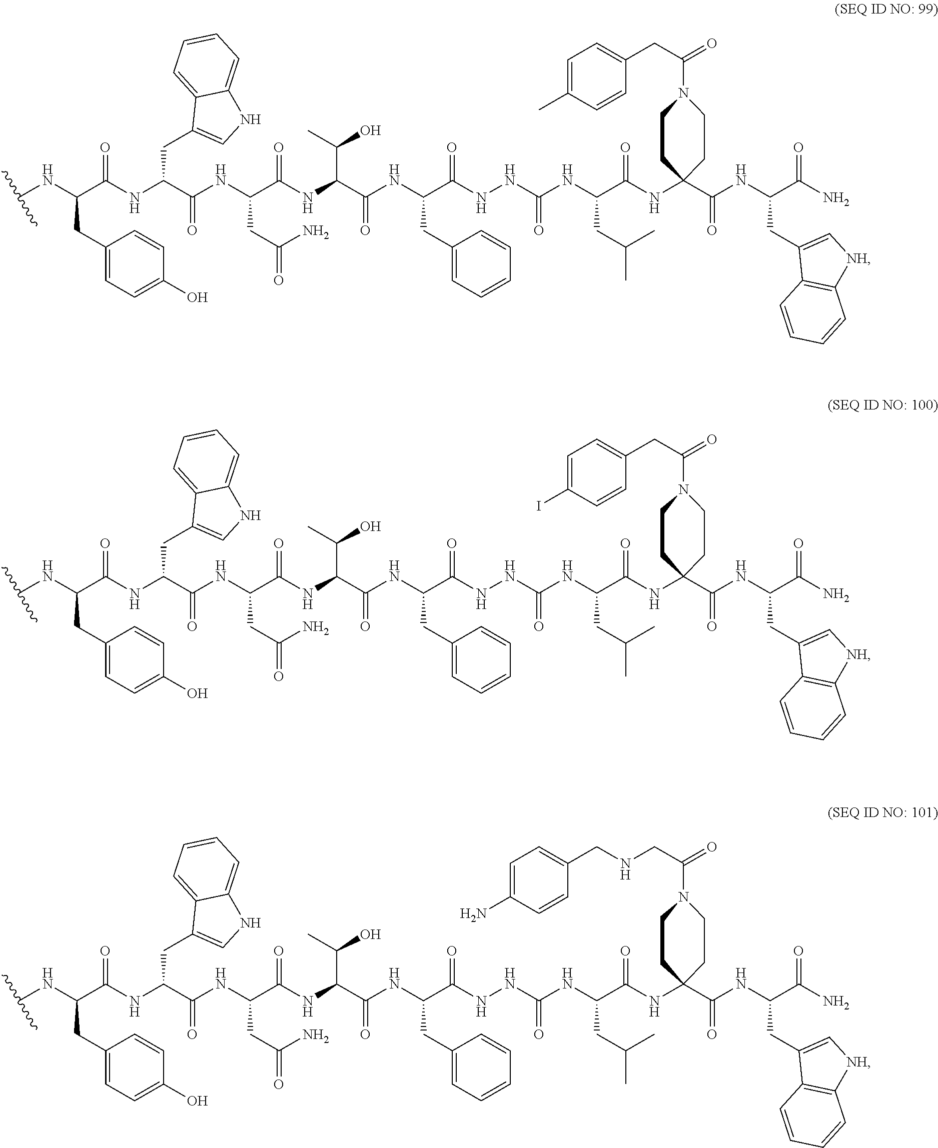

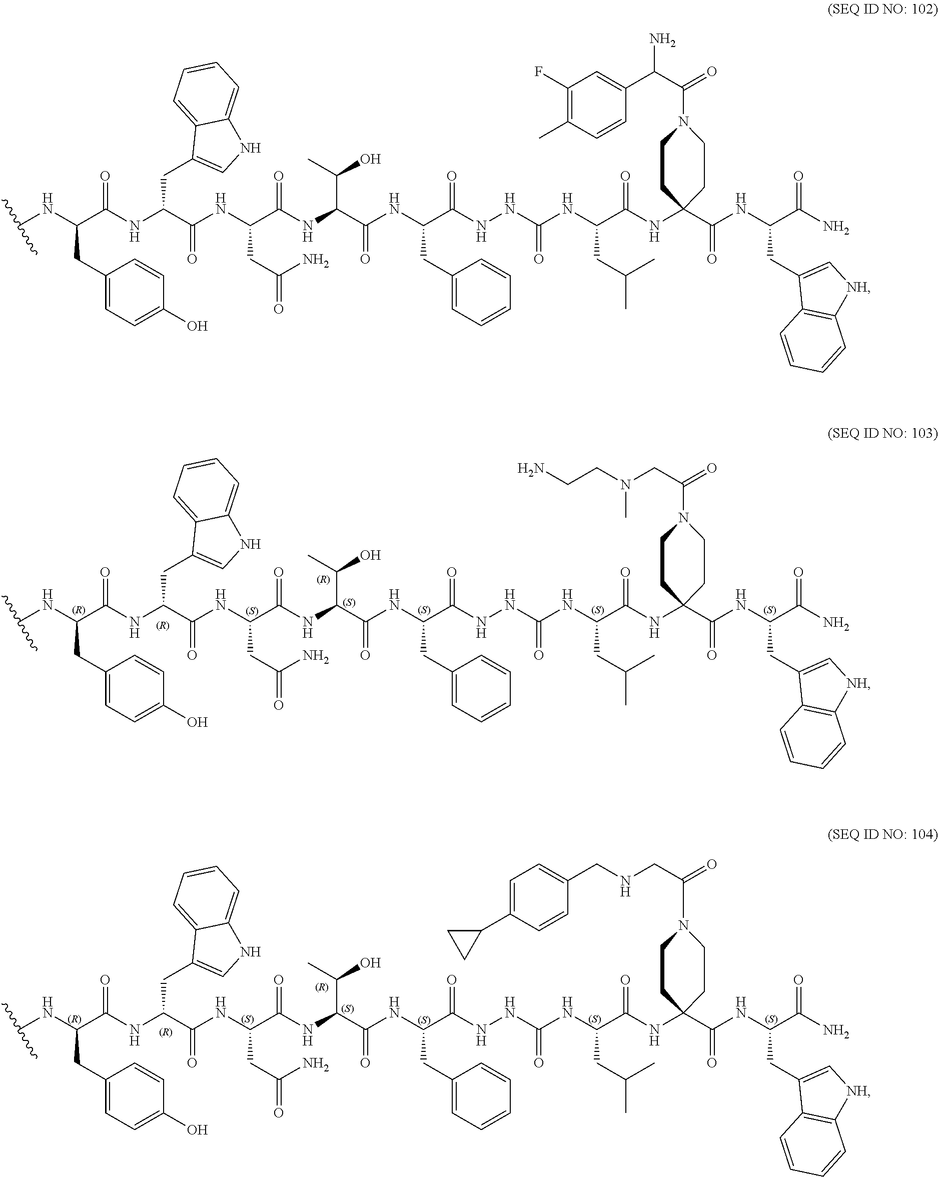

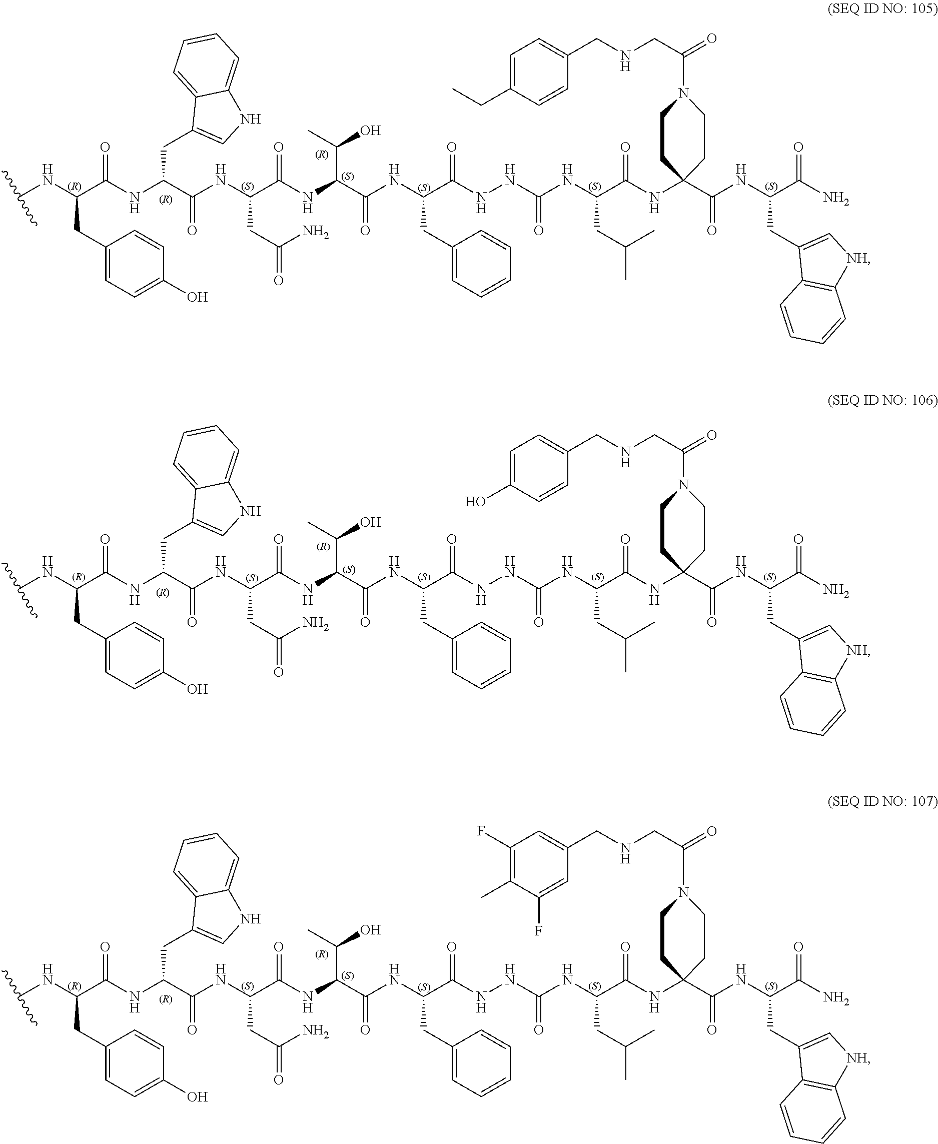

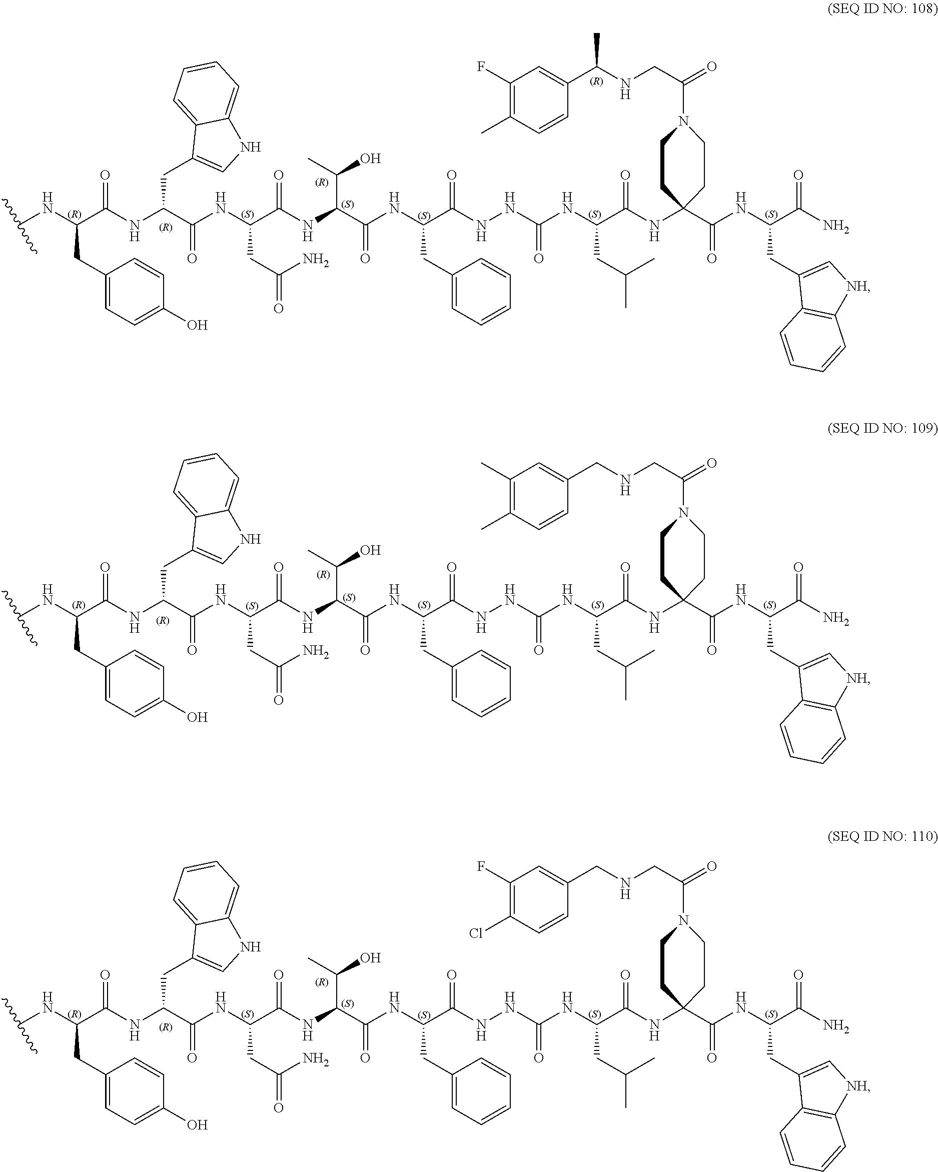

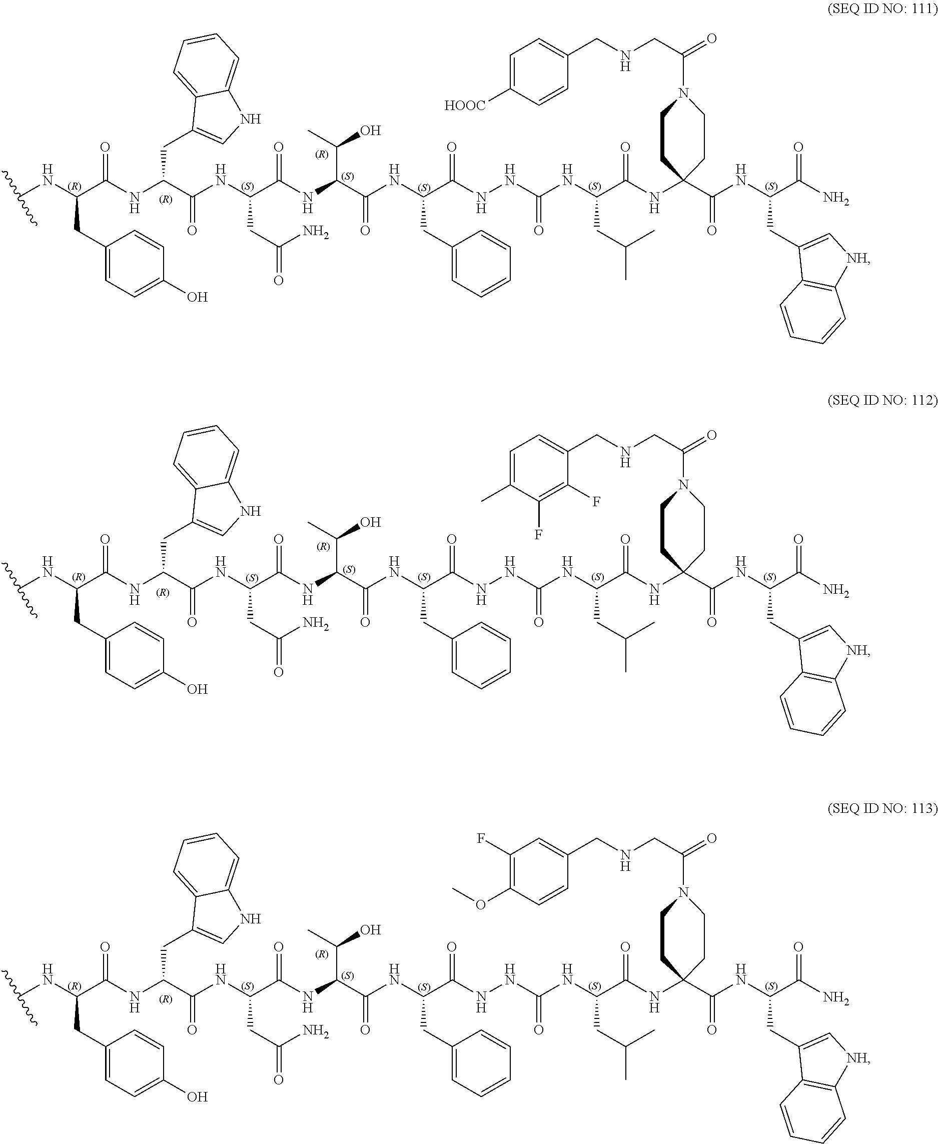

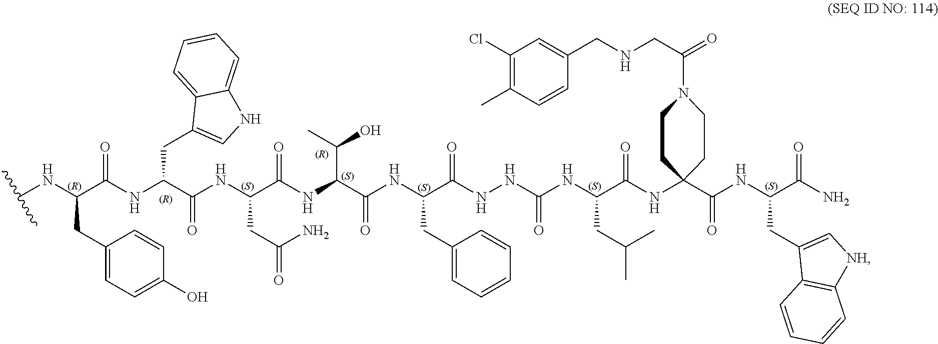

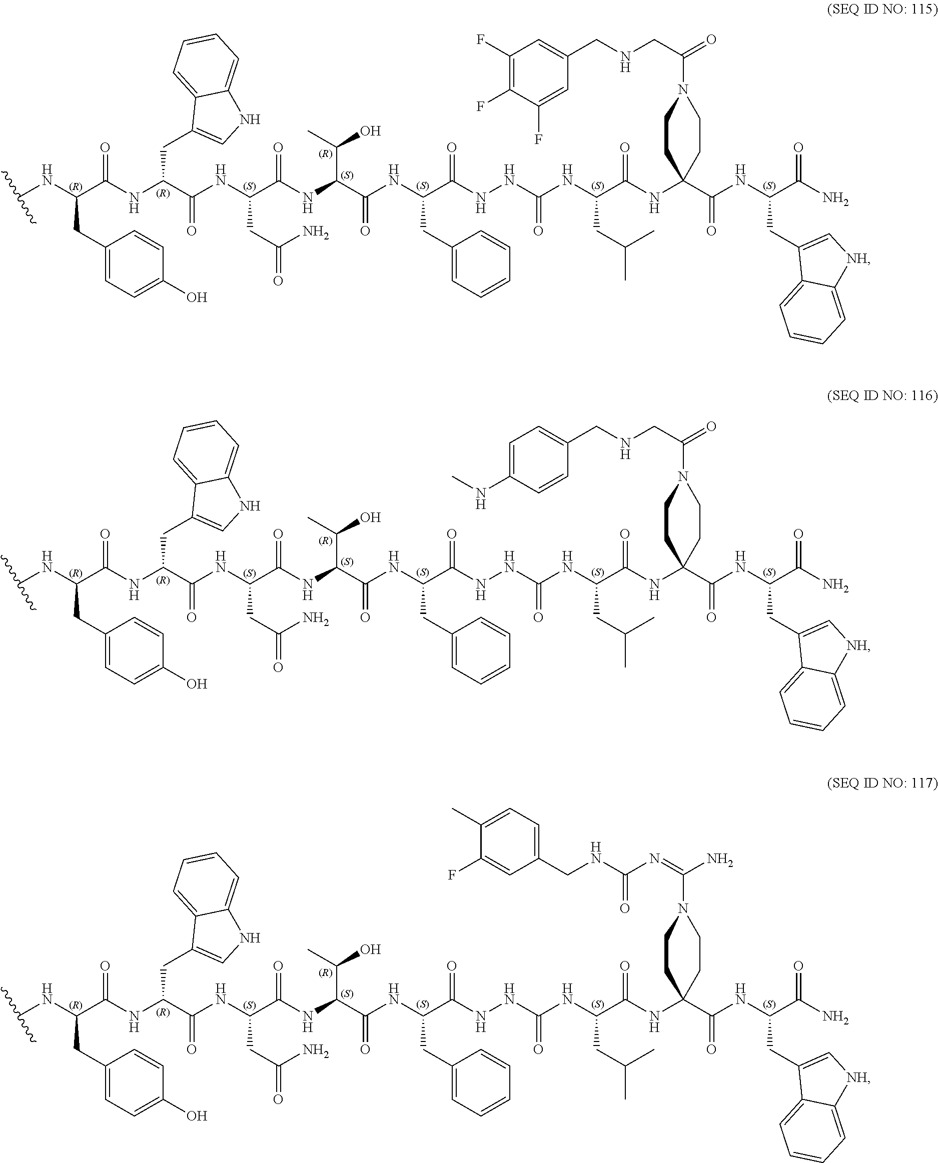

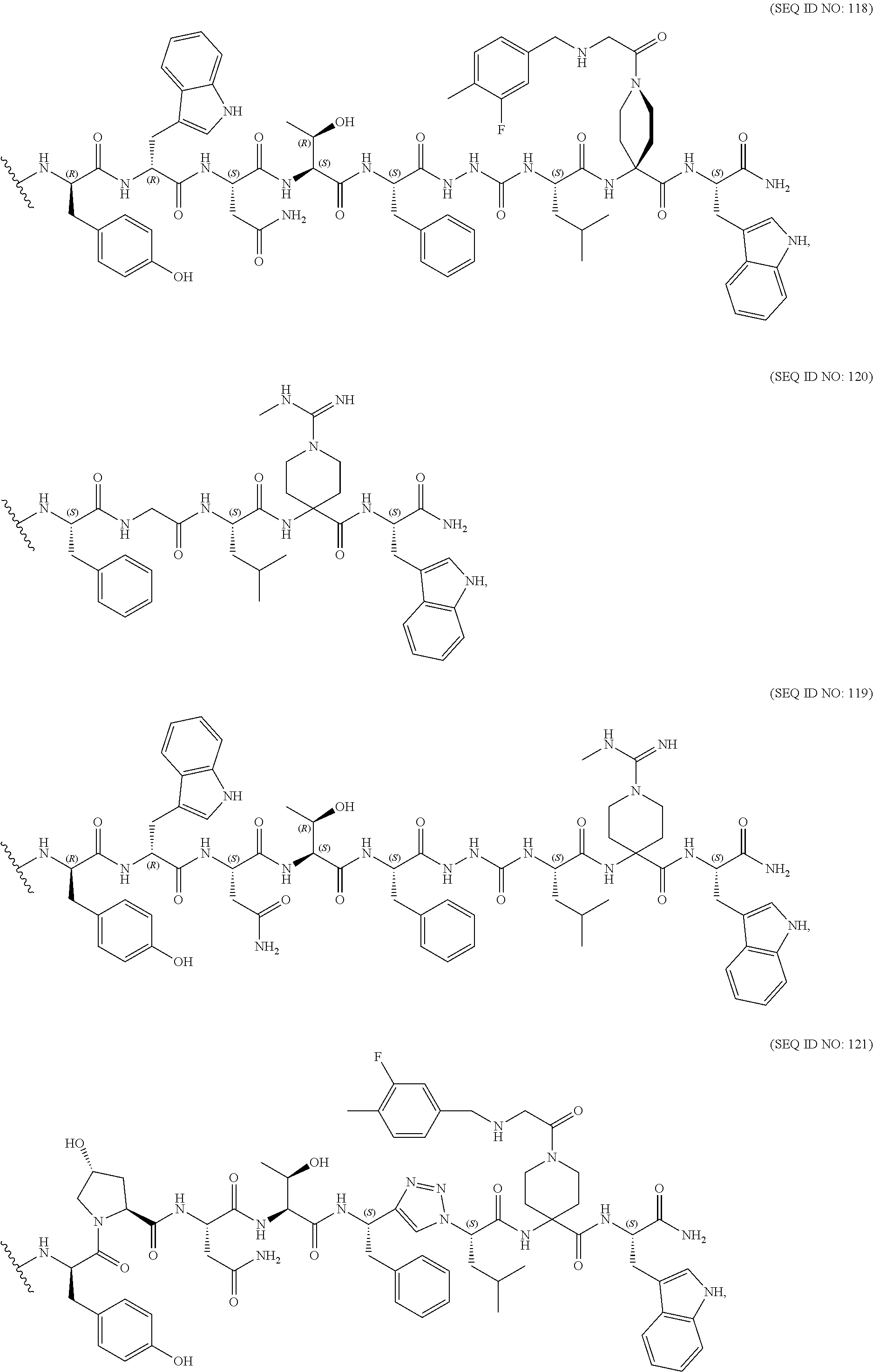

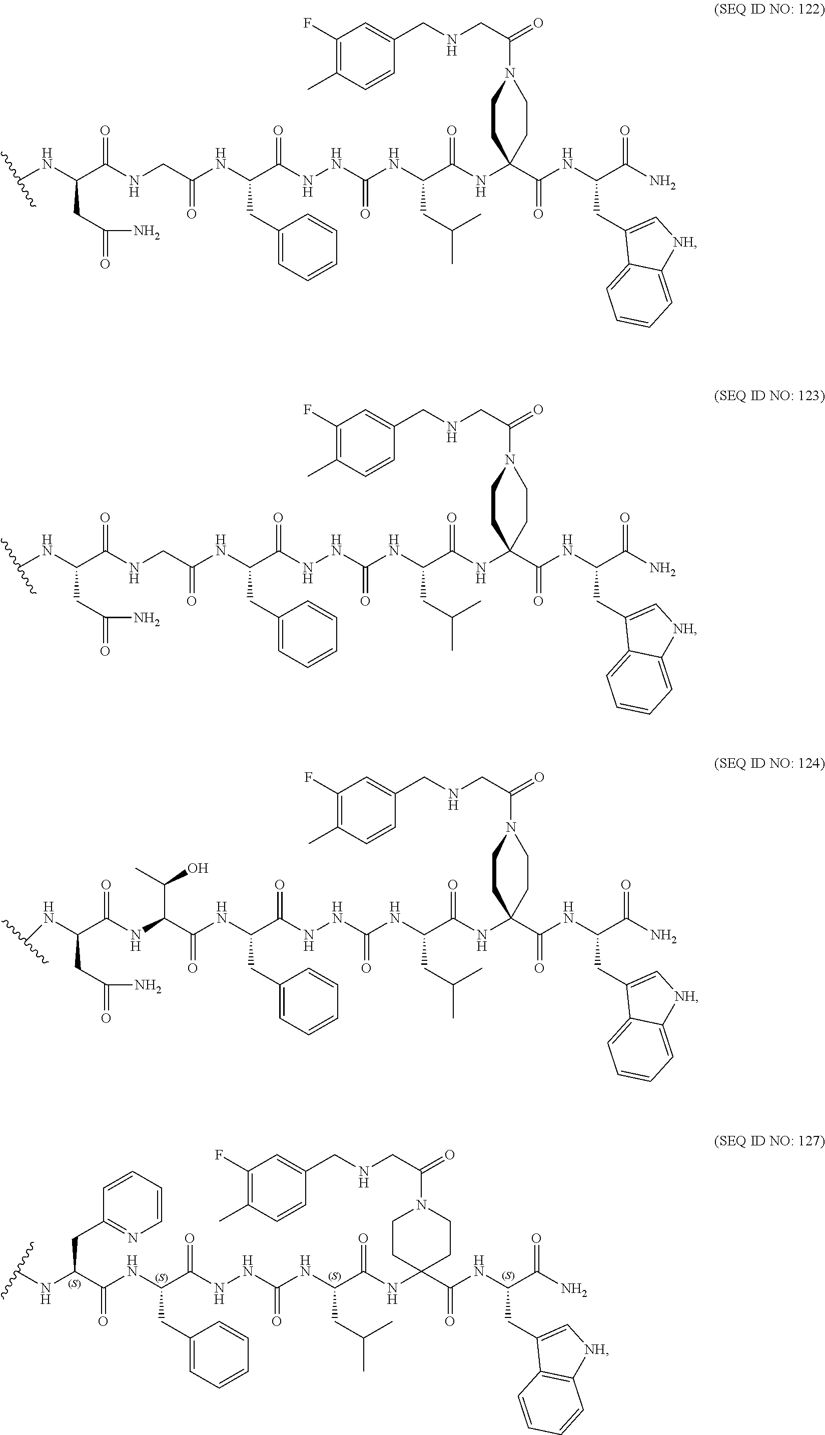

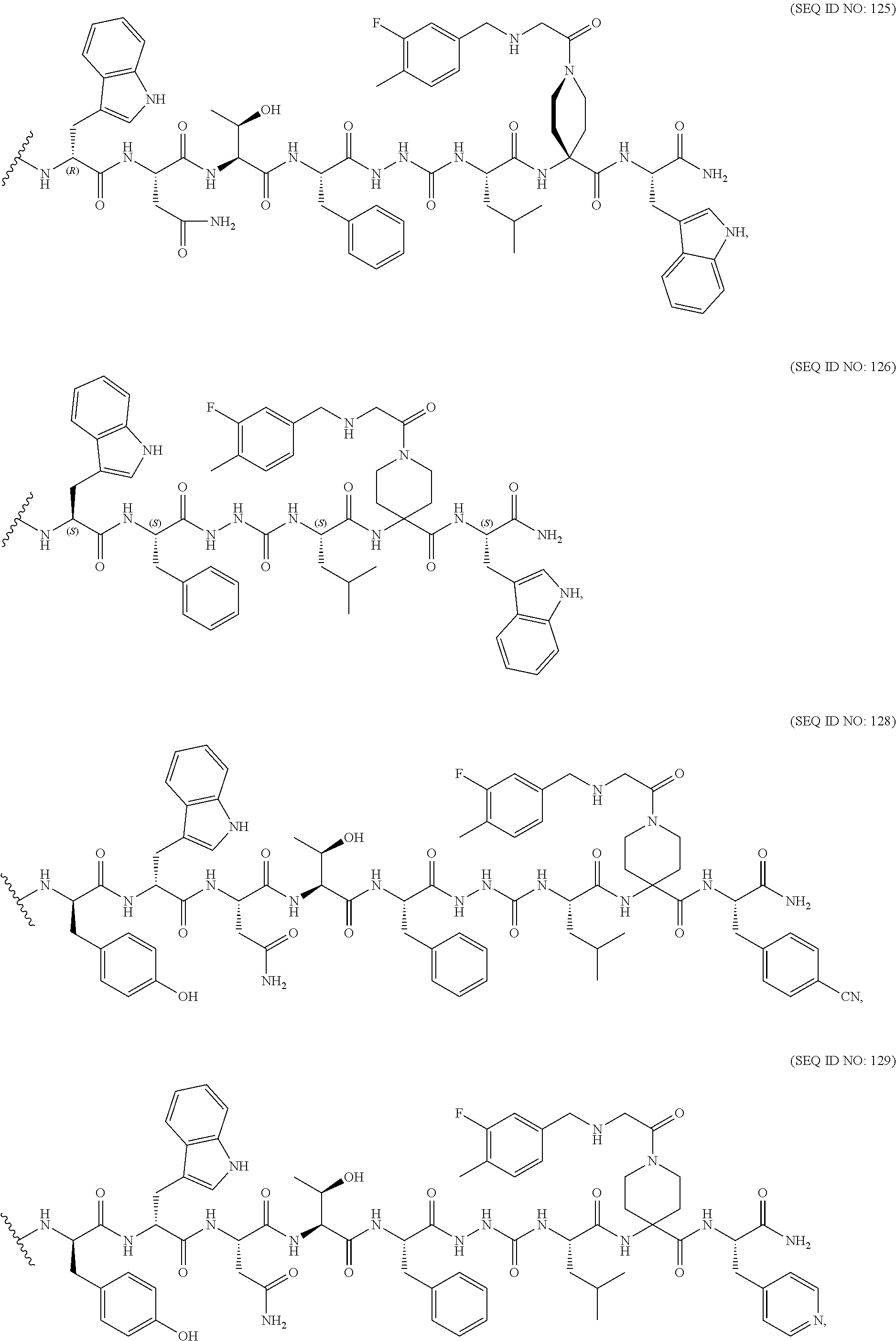

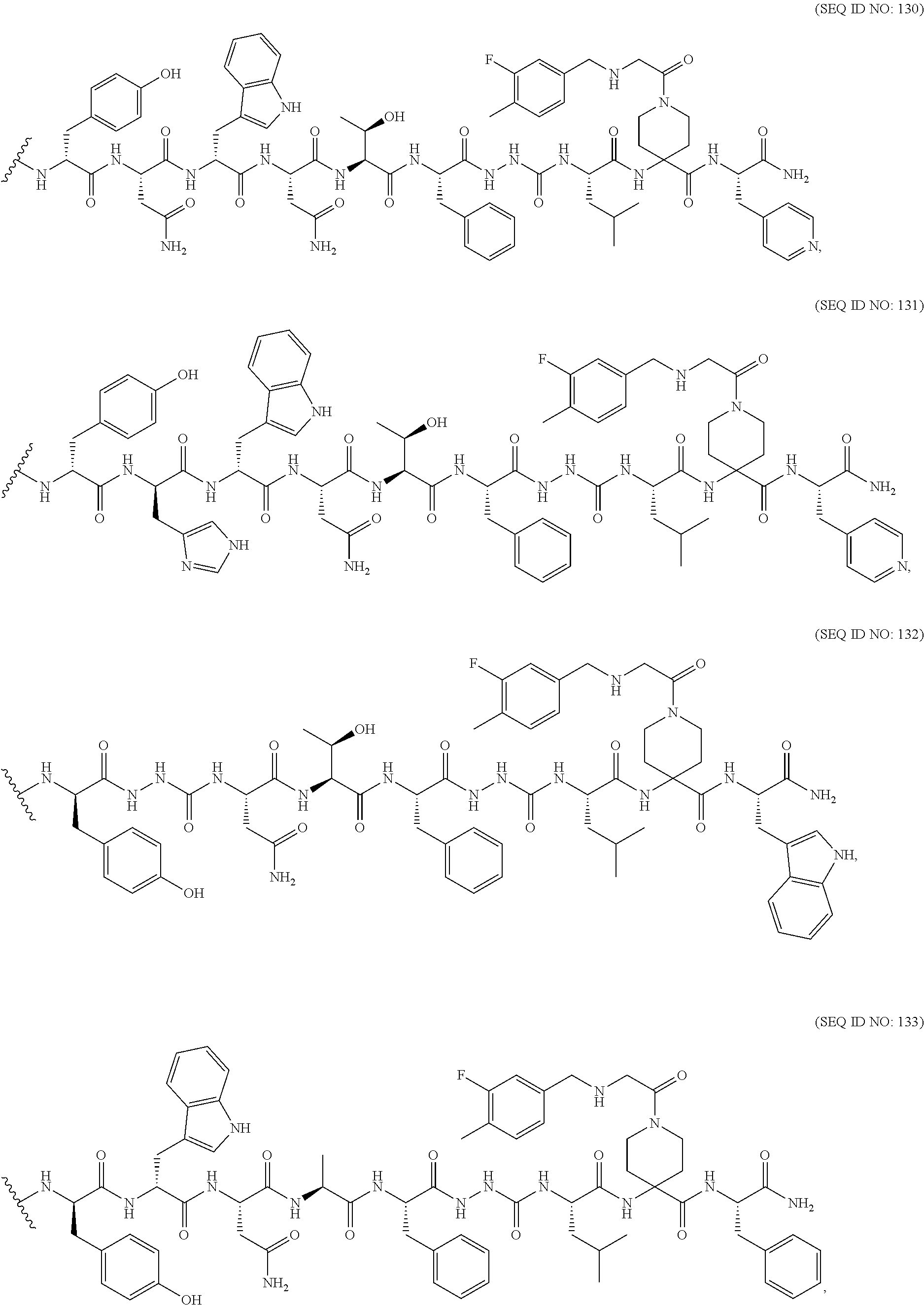

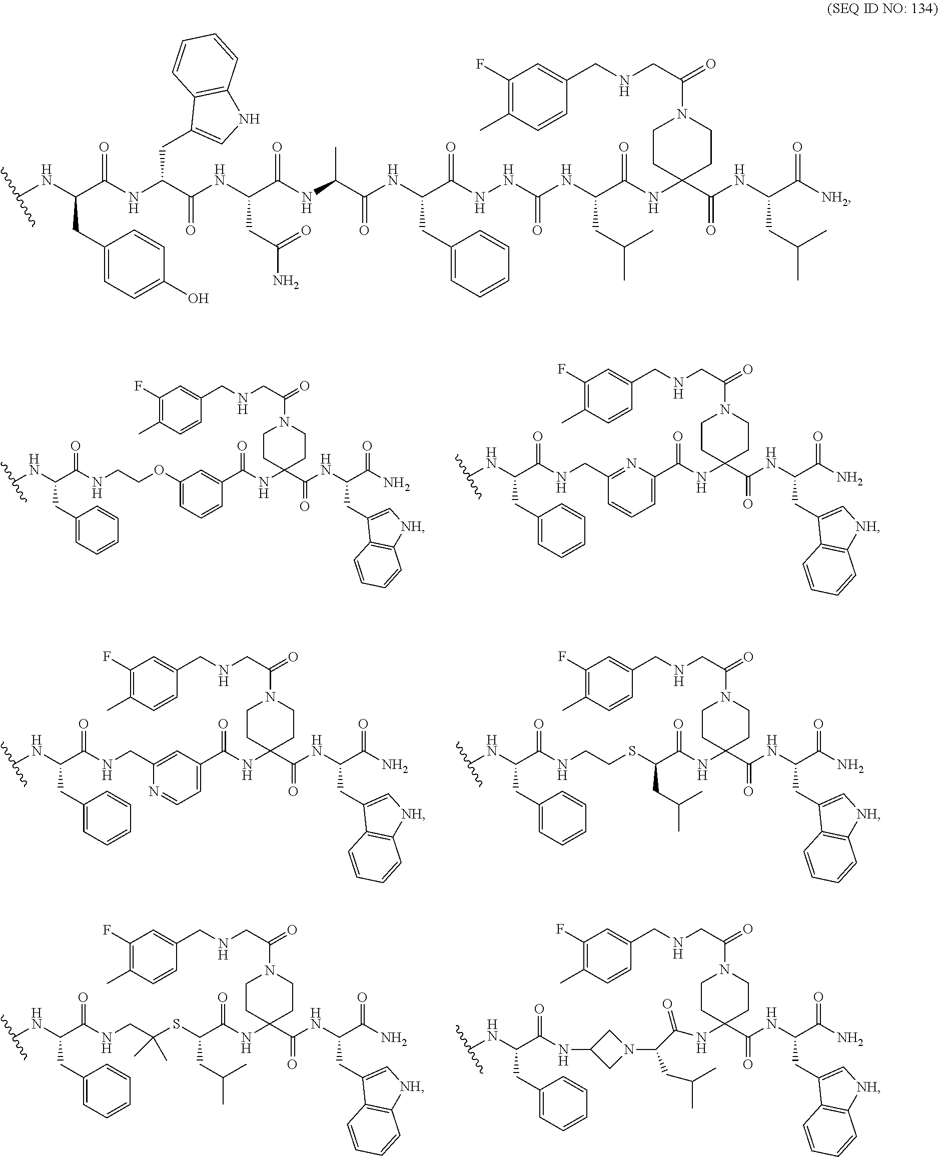

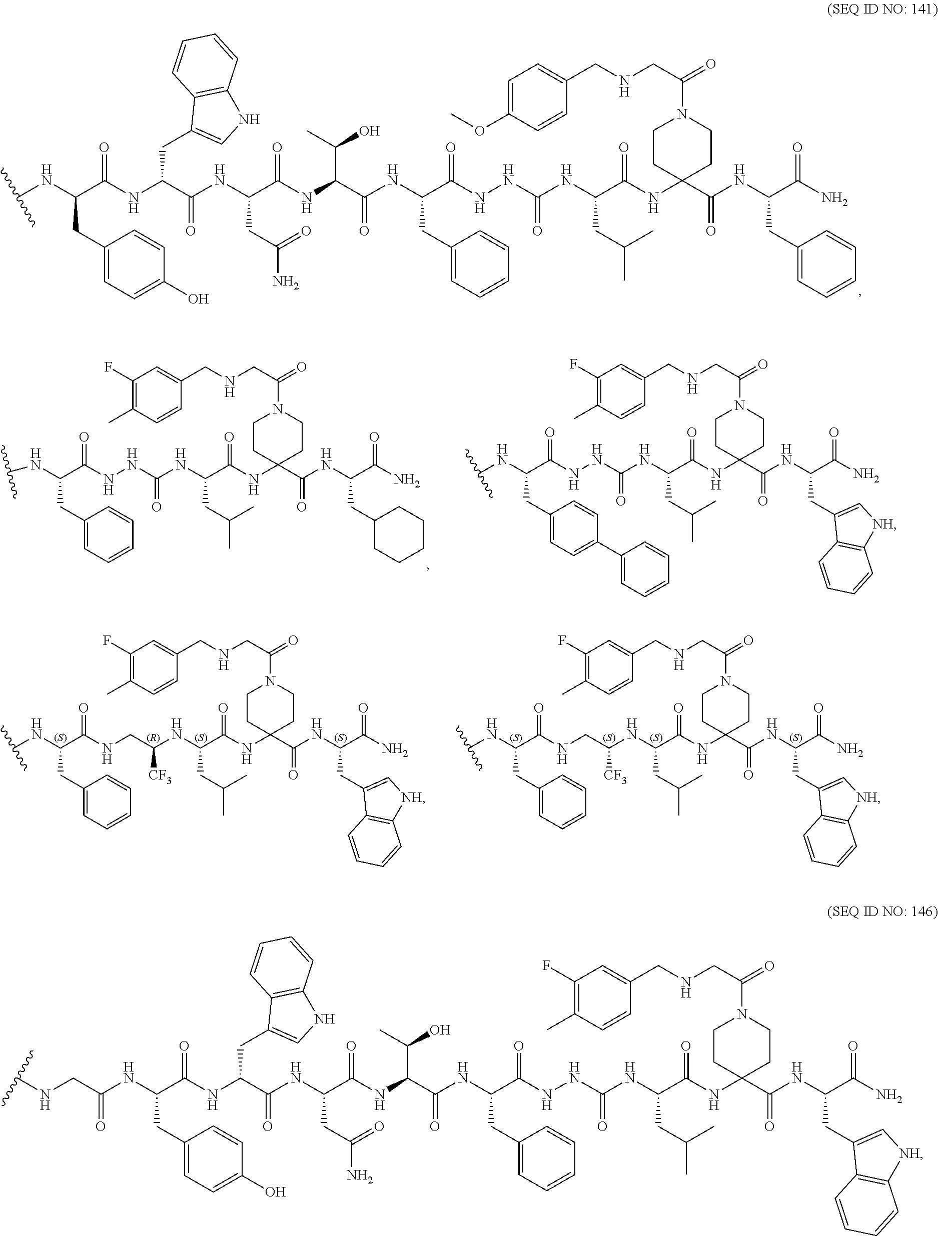

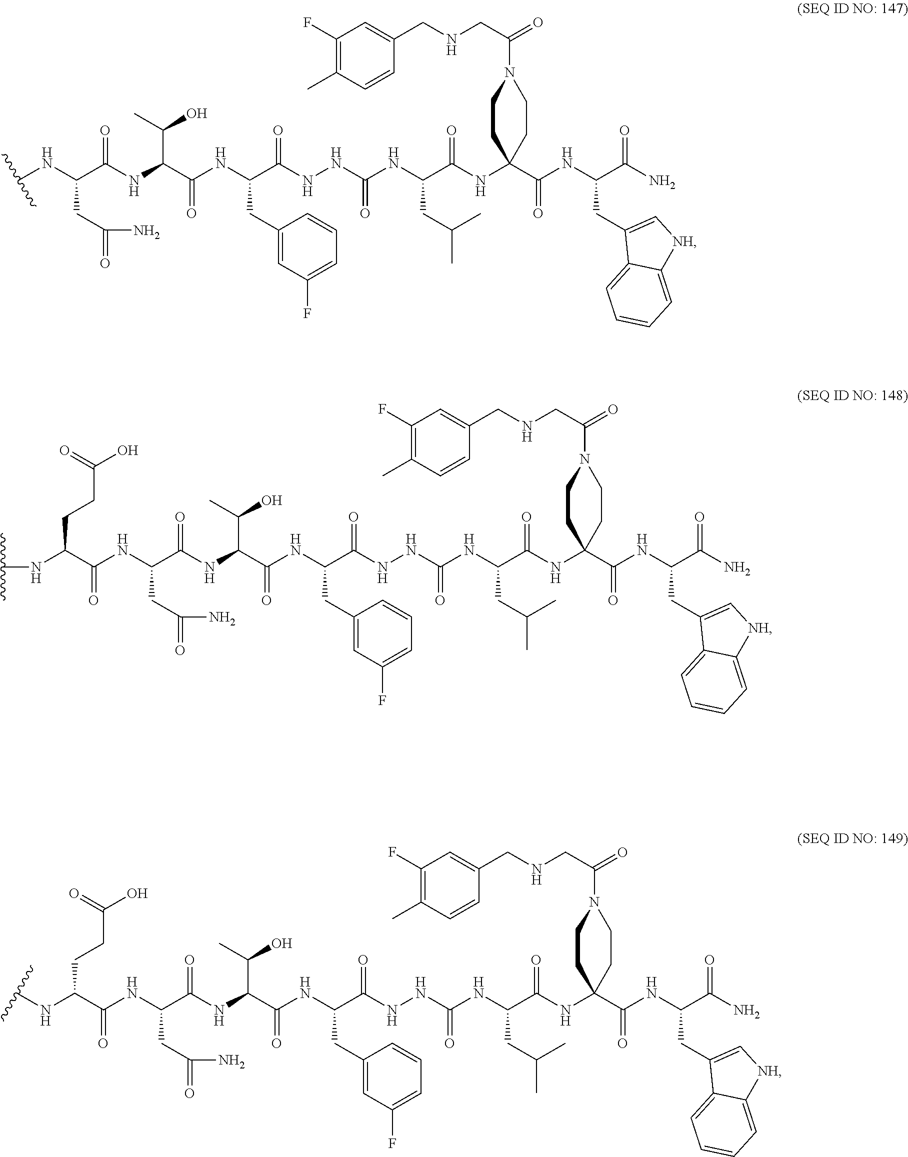

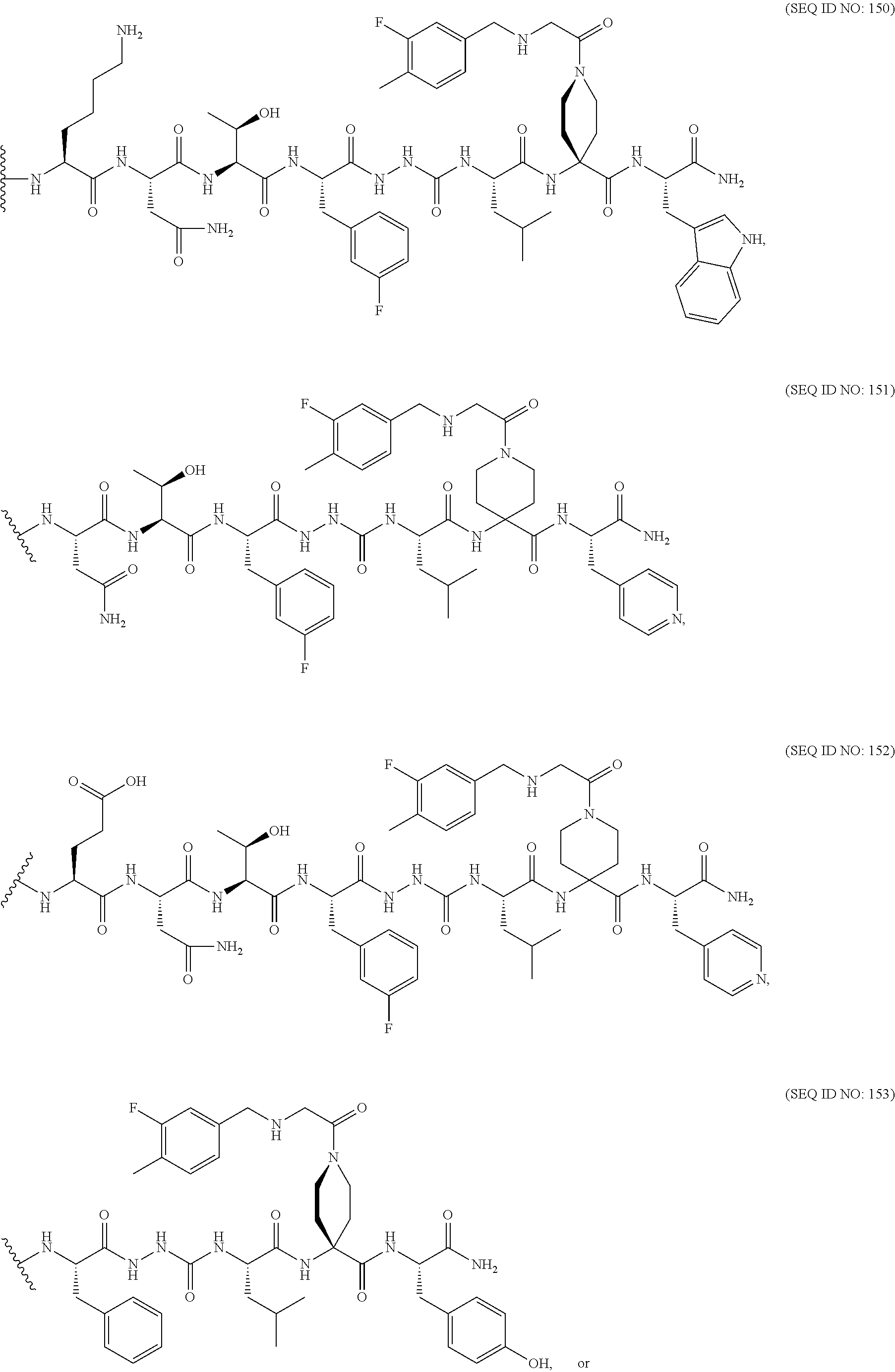

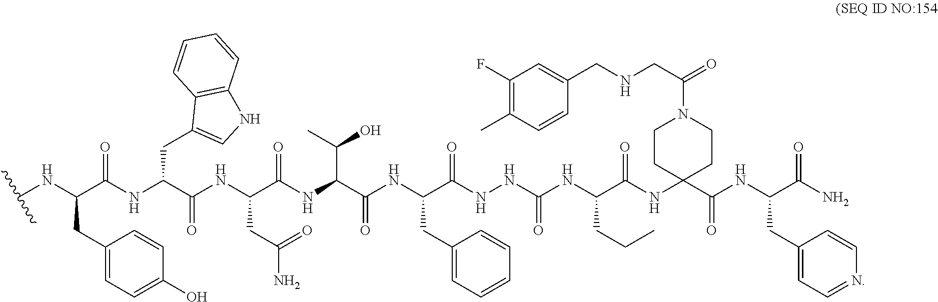 [SMILES: *N[C@H](Cc1ccc(O)cc1)C(=O)N[C@H](Cc1c[nH]c2ccccc12)C(=O)N[C@@H](CC(N)=O)C(=O)N[C@H](C(=O)N[C@@H](Cc1ccccc1)C(=O)NNC(=O)N[C@@H](CCC)C(=O)NC1(C(=O)N[C@@H](Cc2ccncc2)C(N)=O)CCN(C(=O)CNCc2ccc(C)c(F)c2)CC1)[C@@H](C)O]

Embodiment 45. The compound of embodiment 1, or a pharmaceutically acceptable salt thereof, wherein:

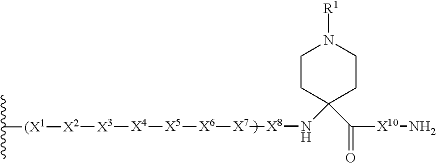

is

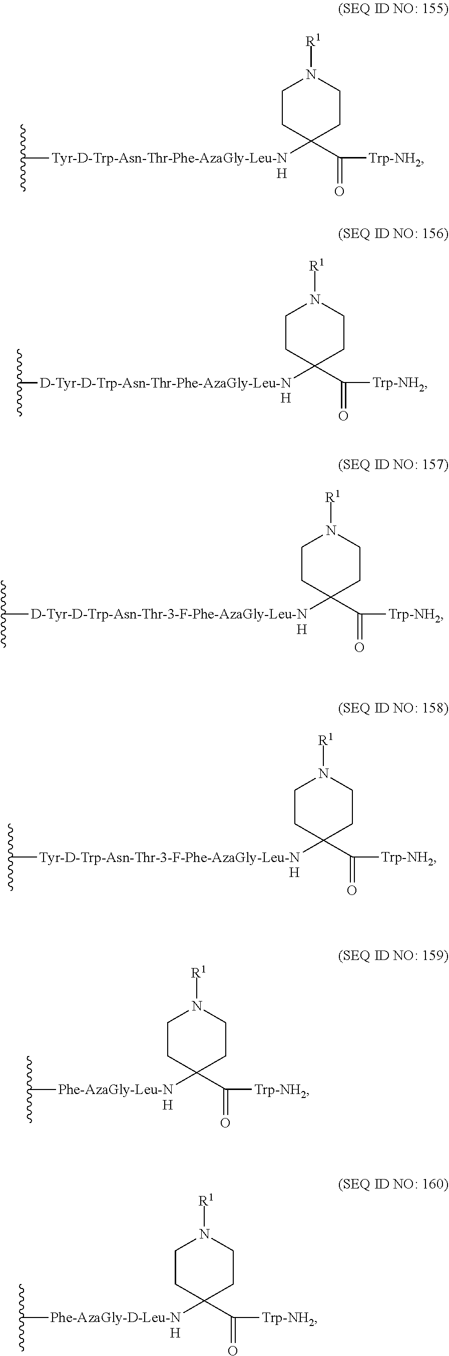

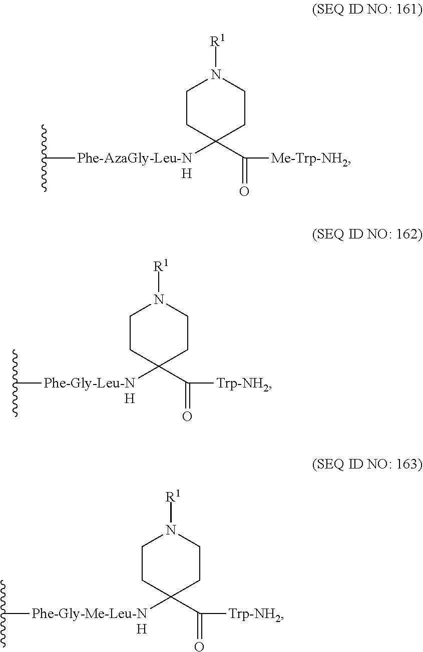

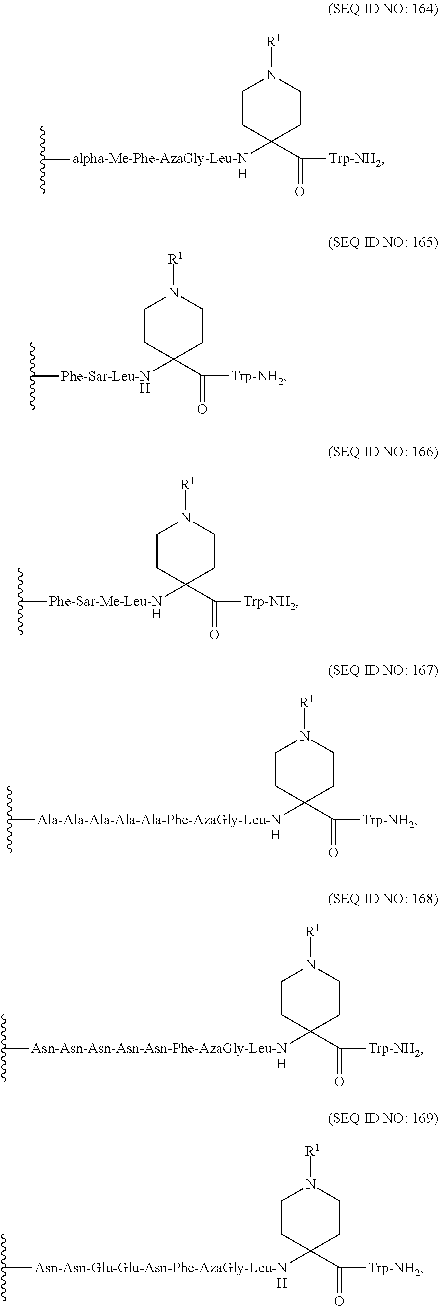

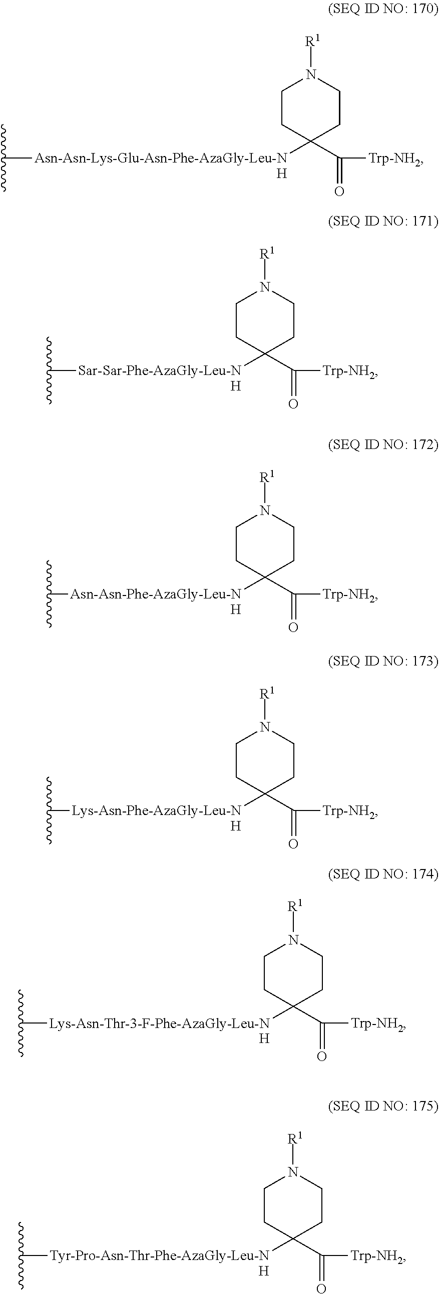

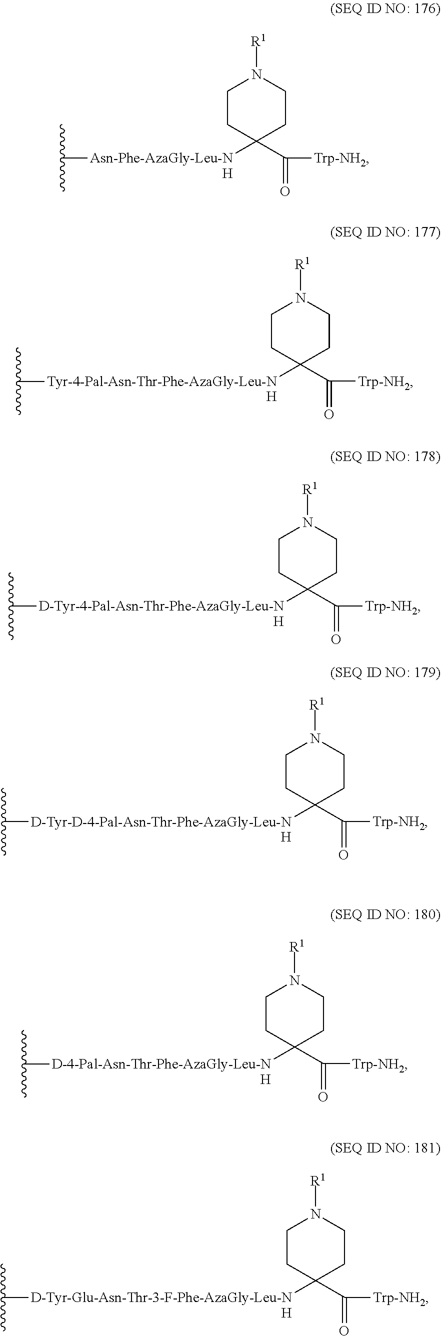

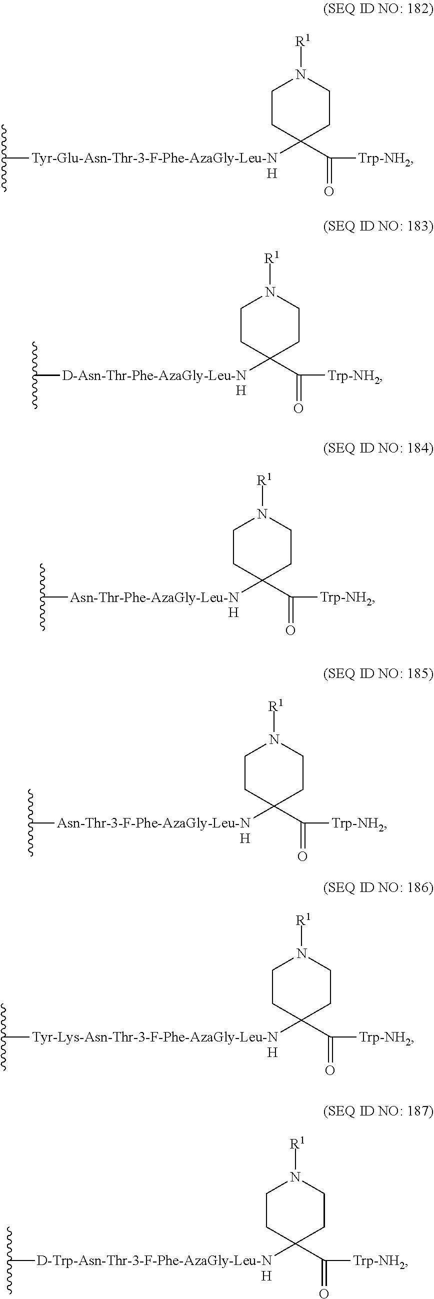

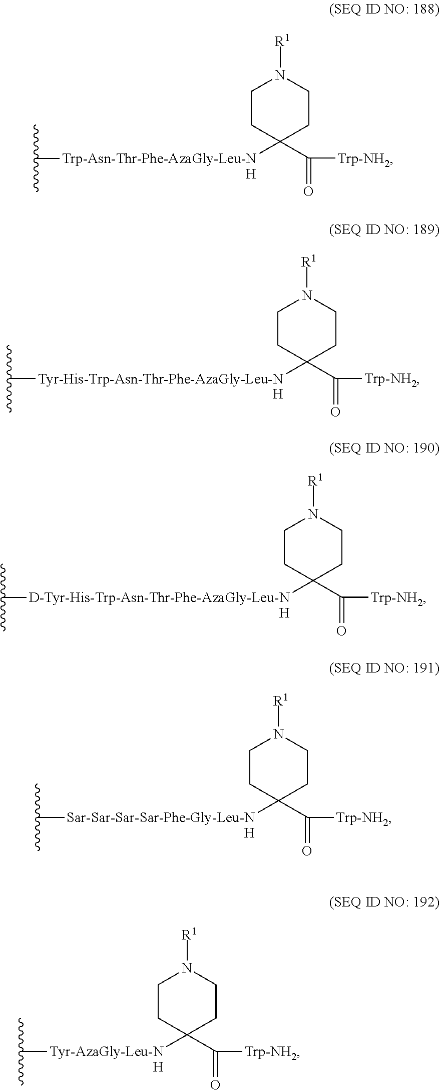

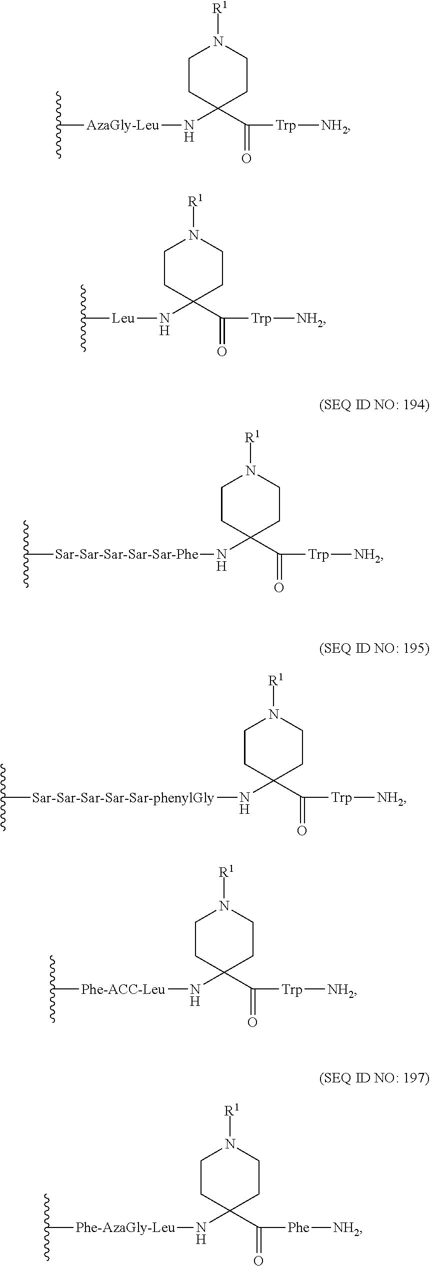

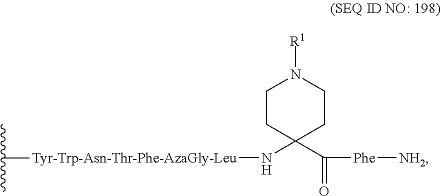

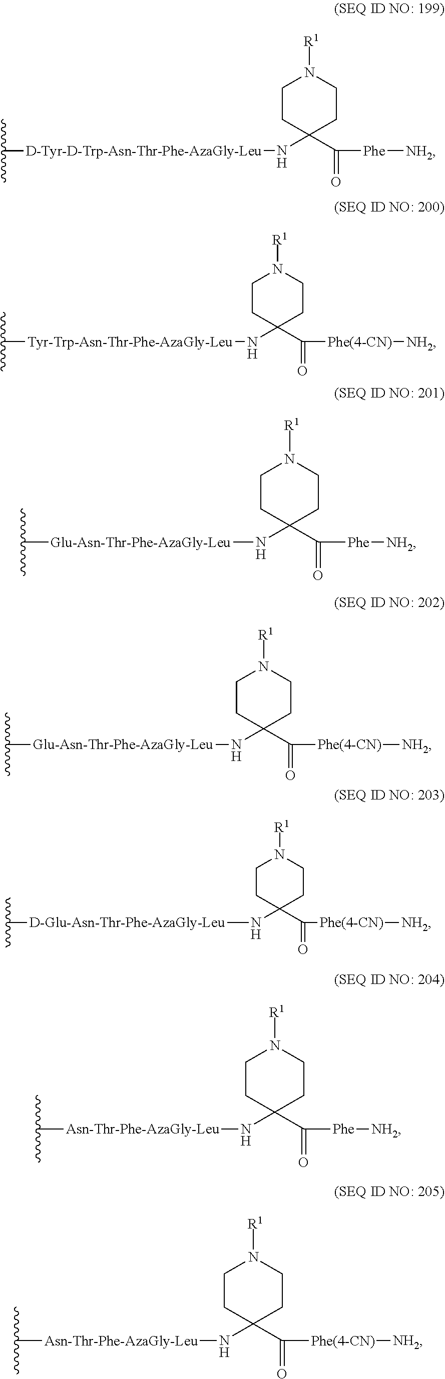

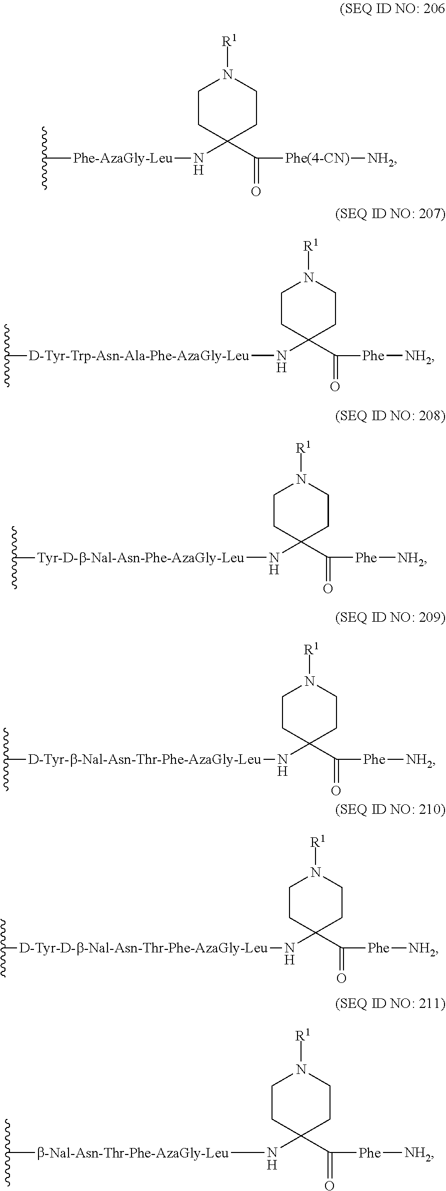

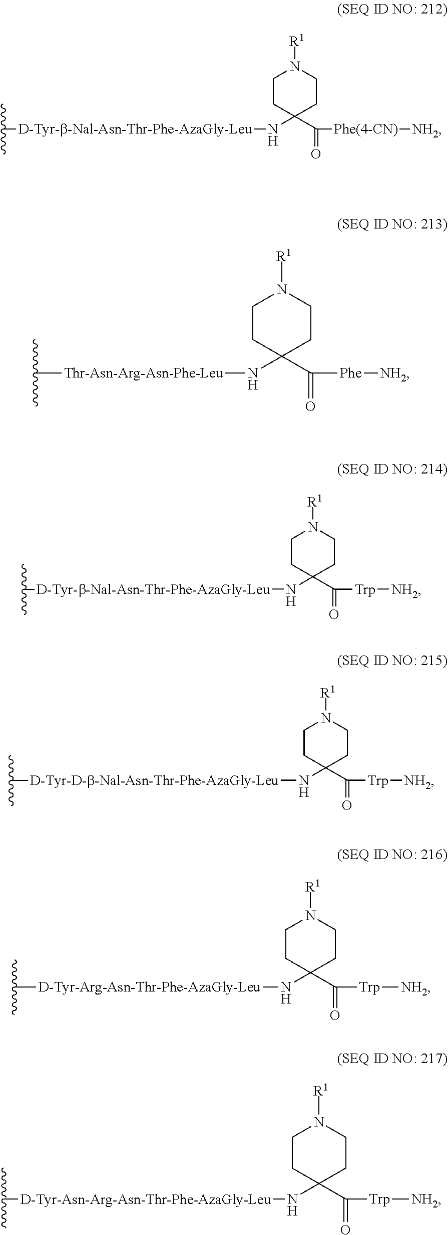

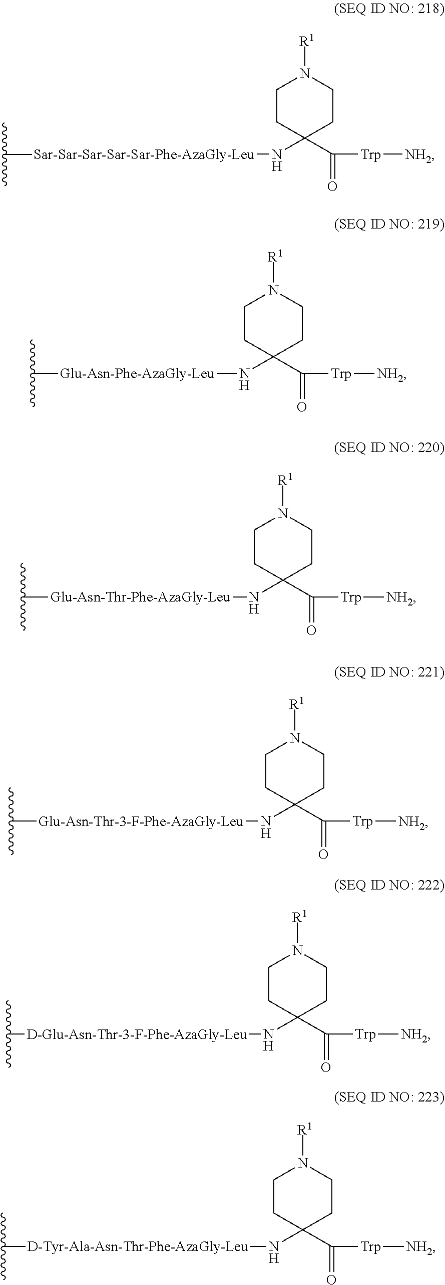

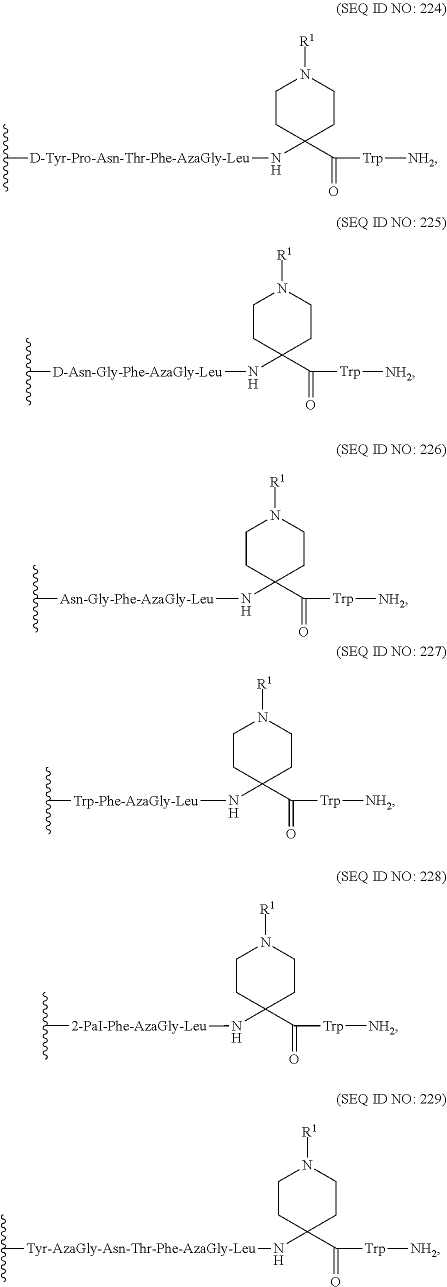

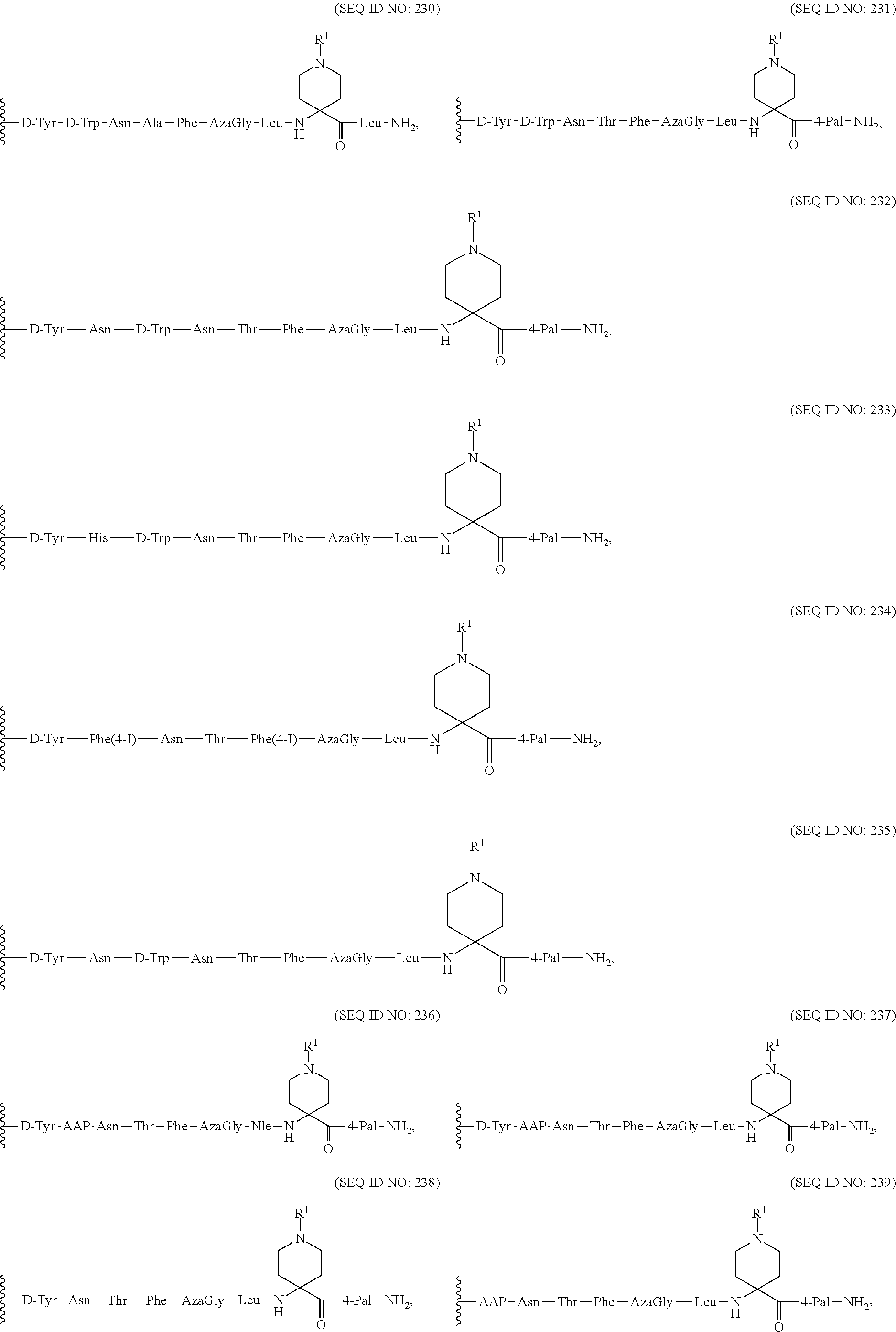

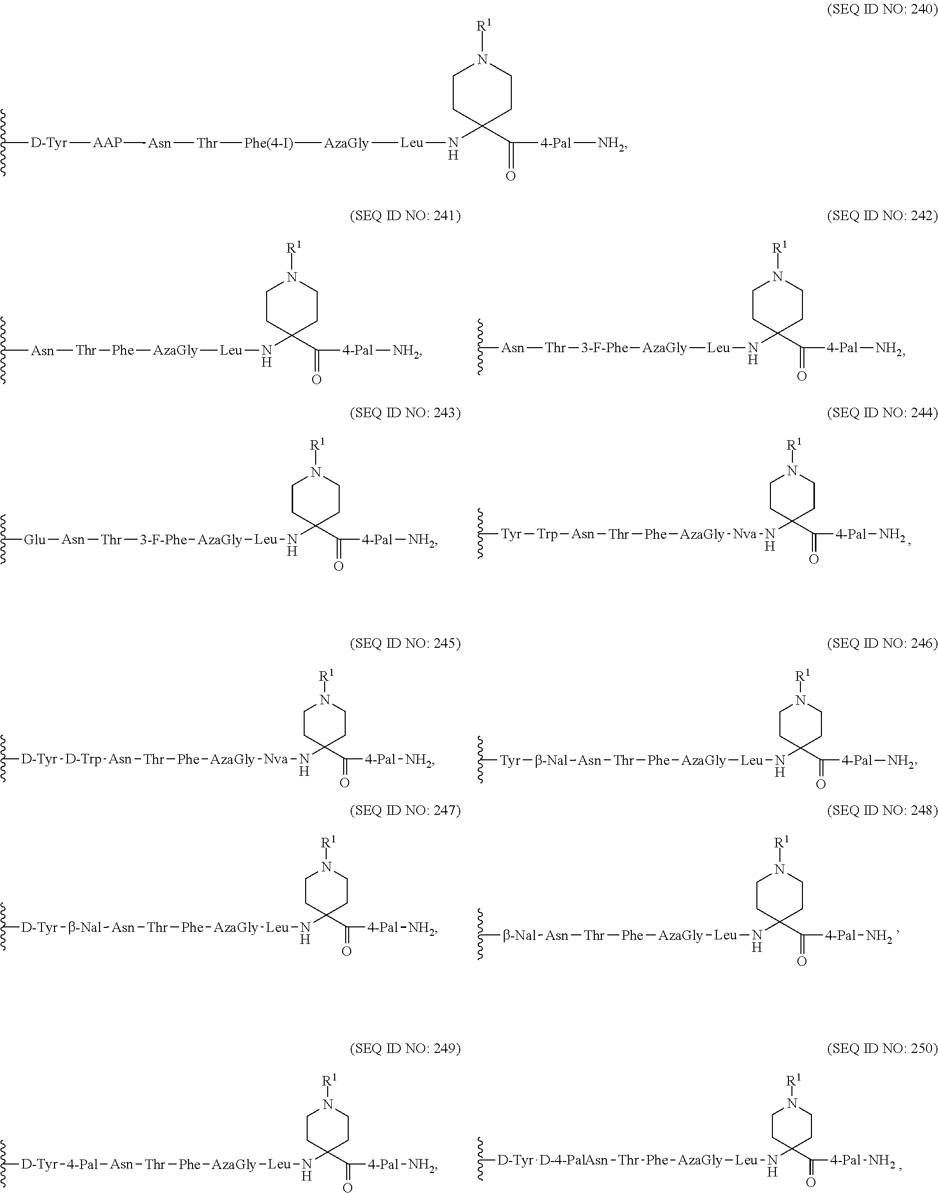

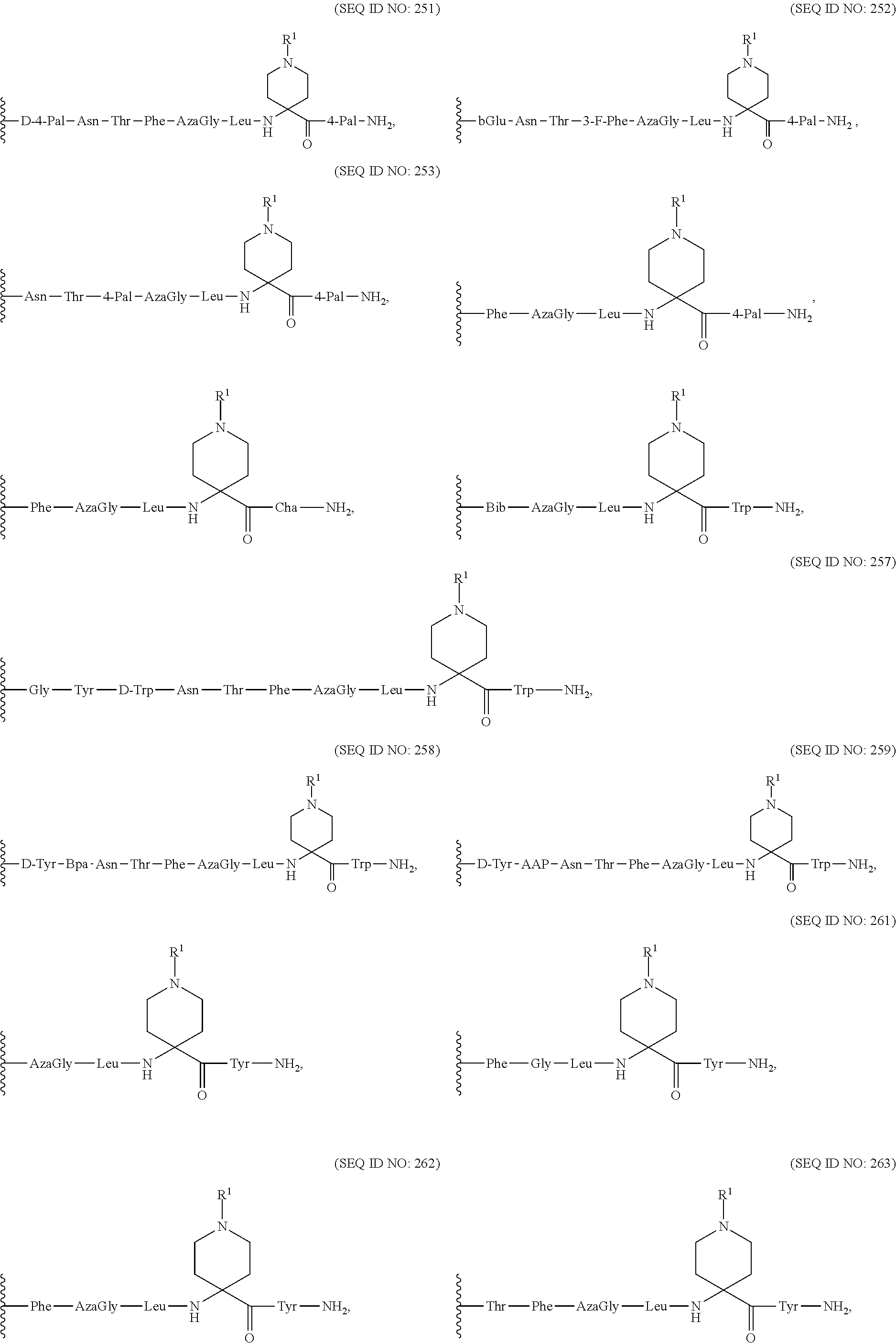

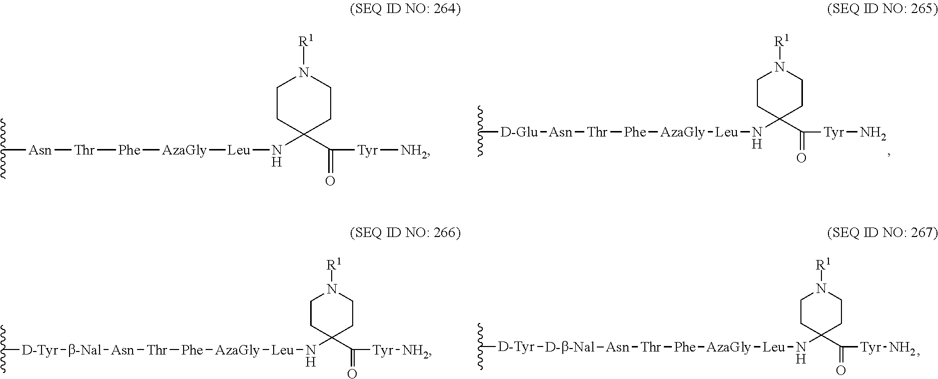

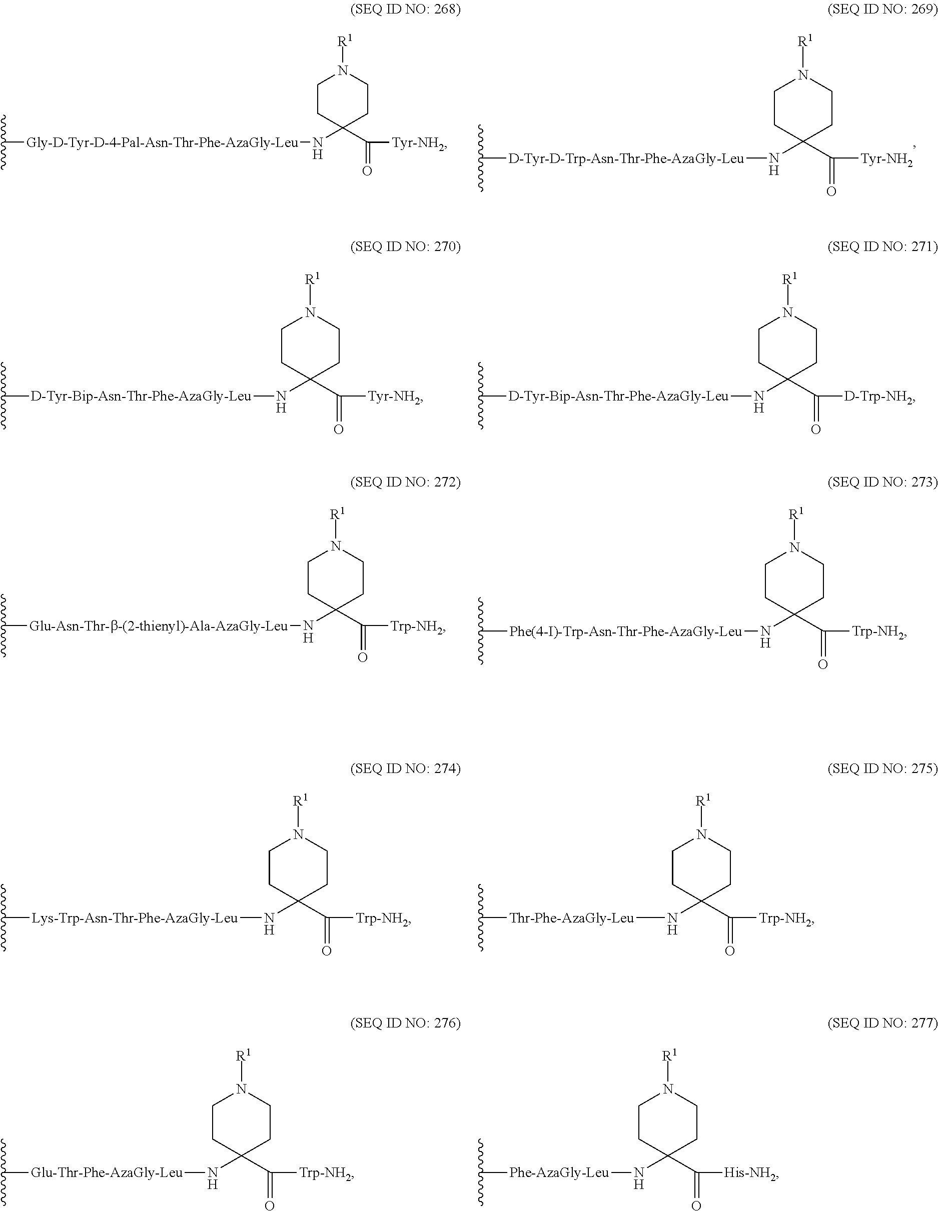

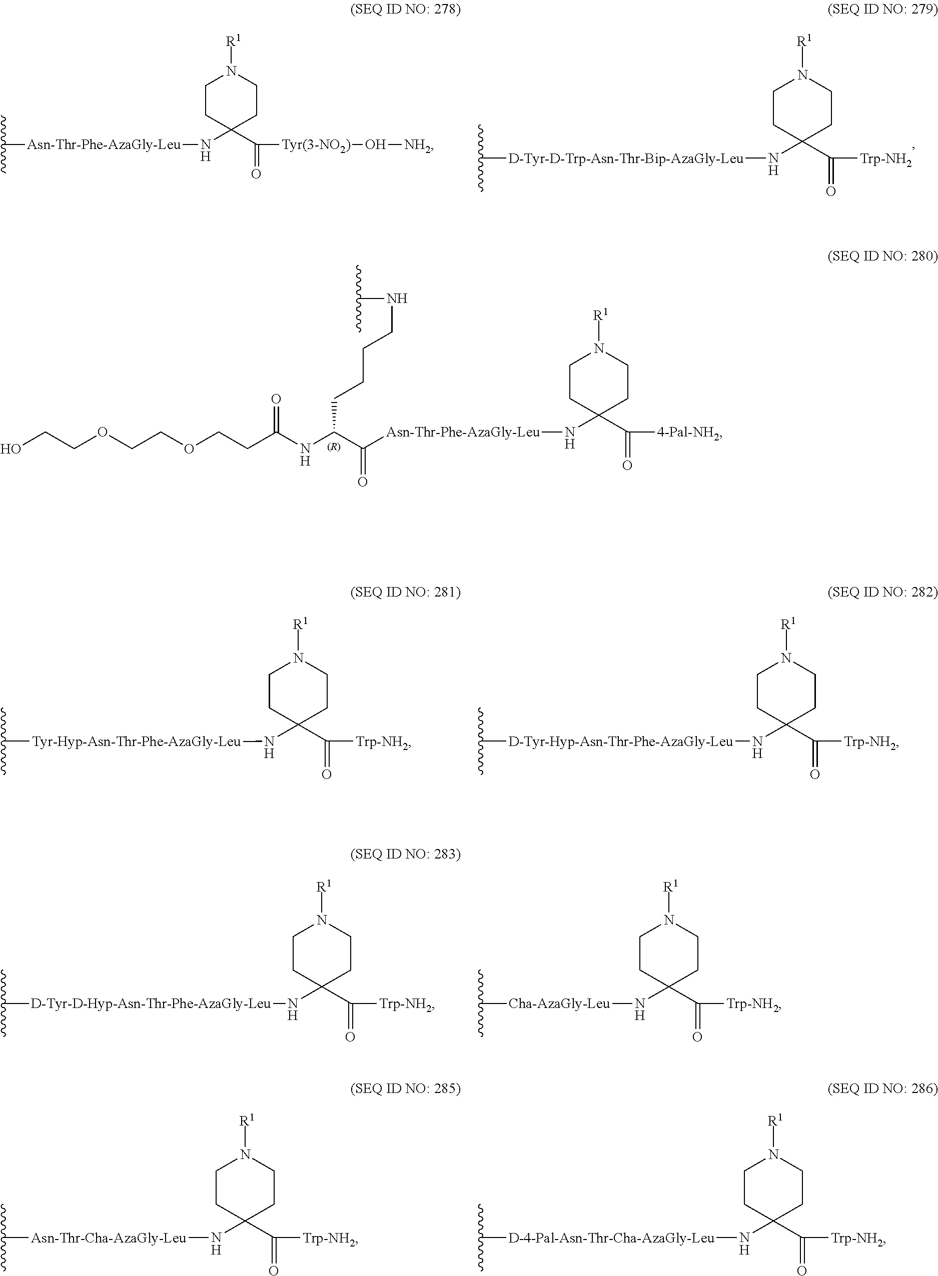

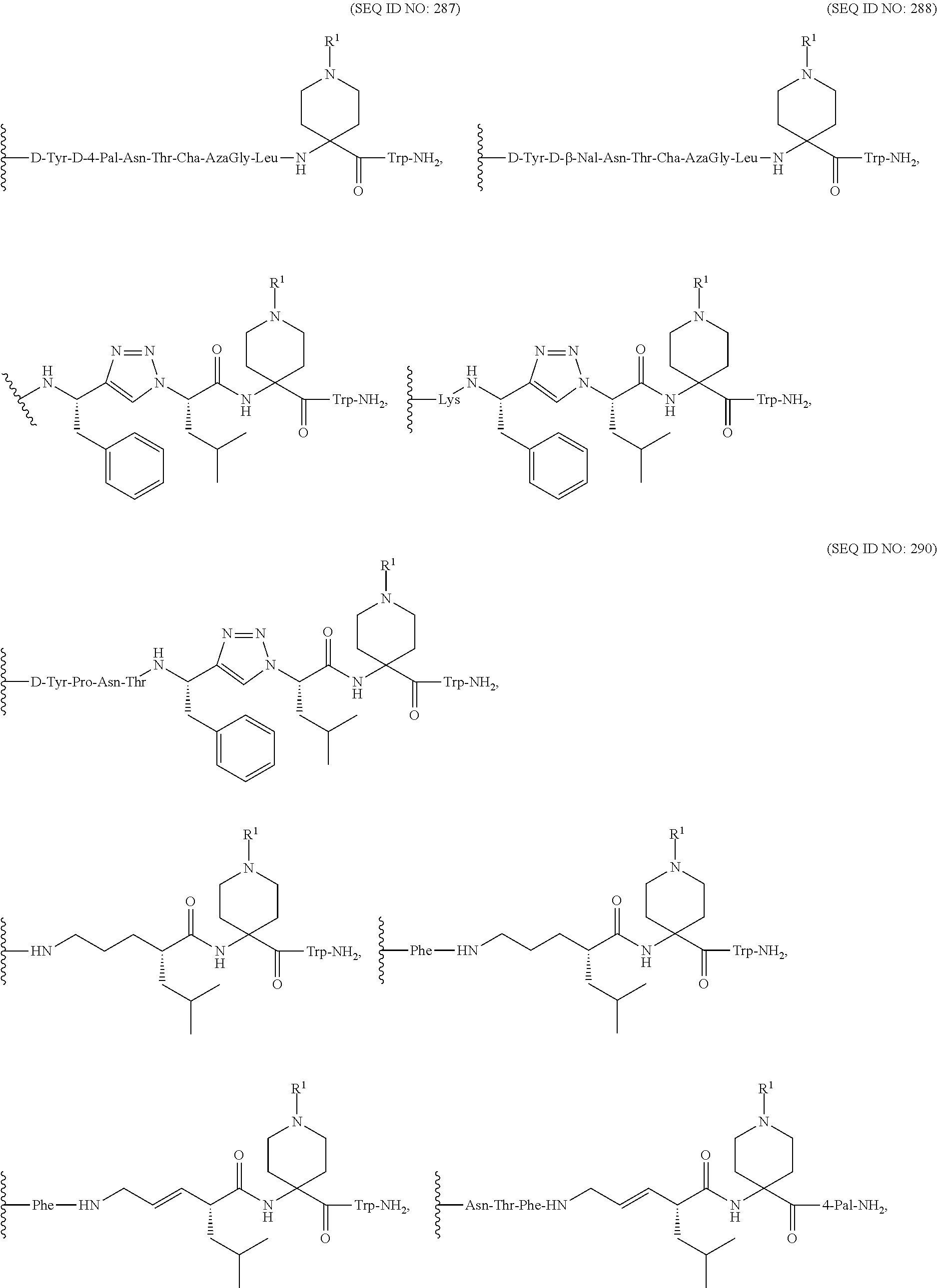

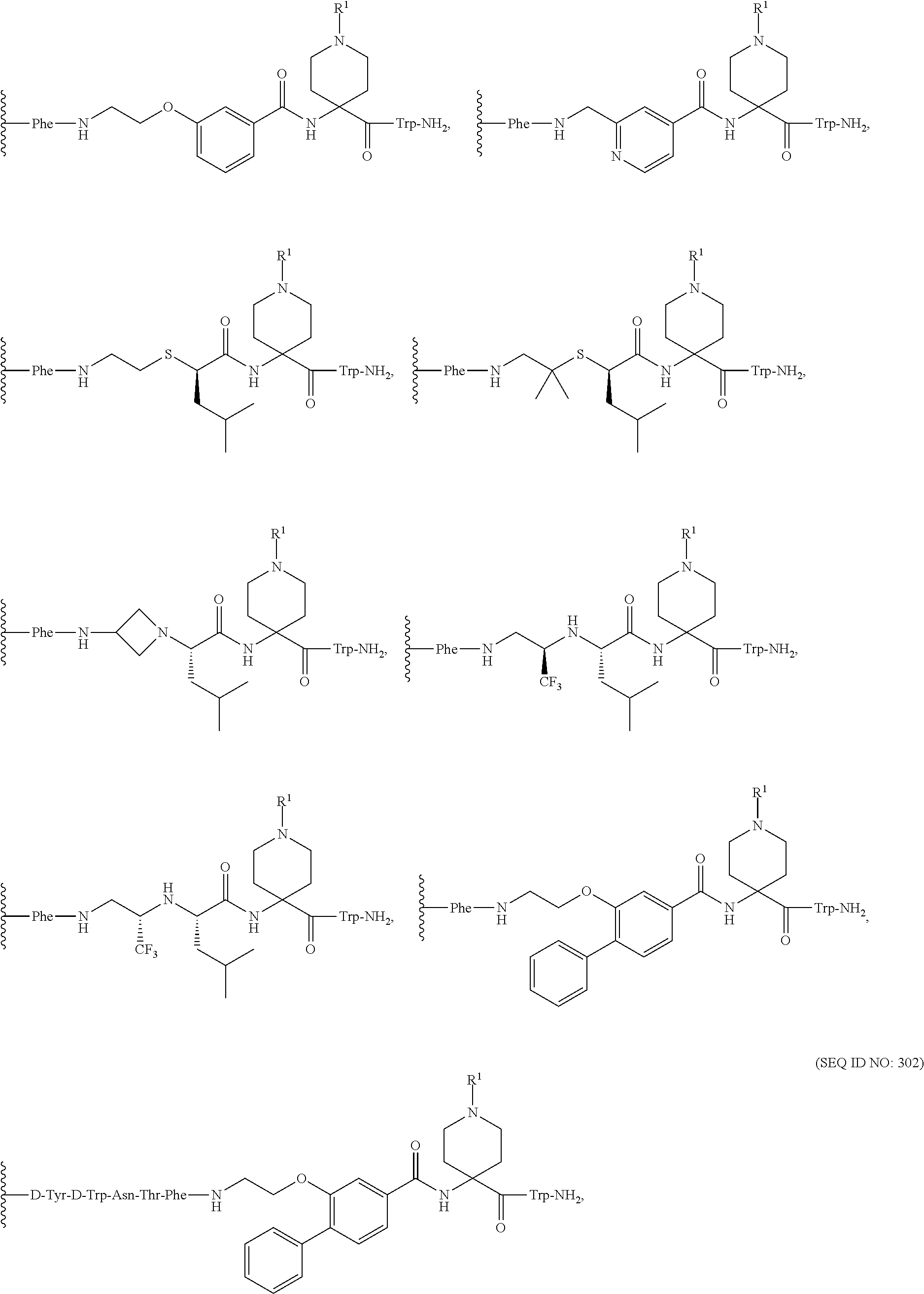

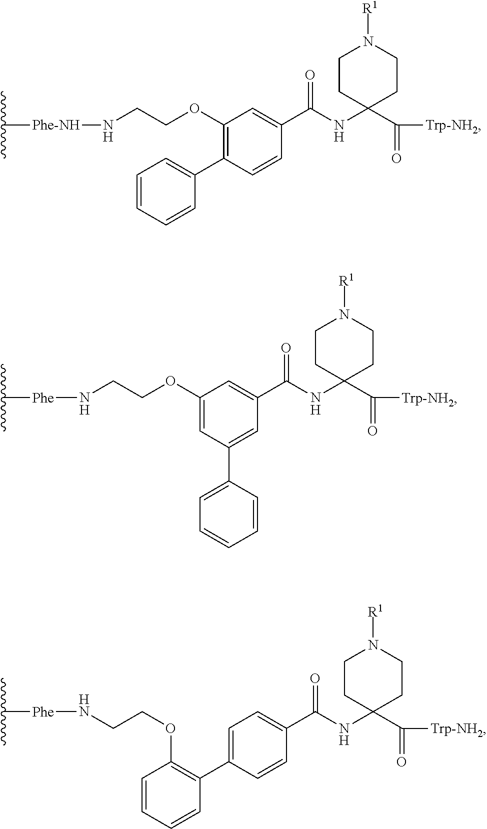

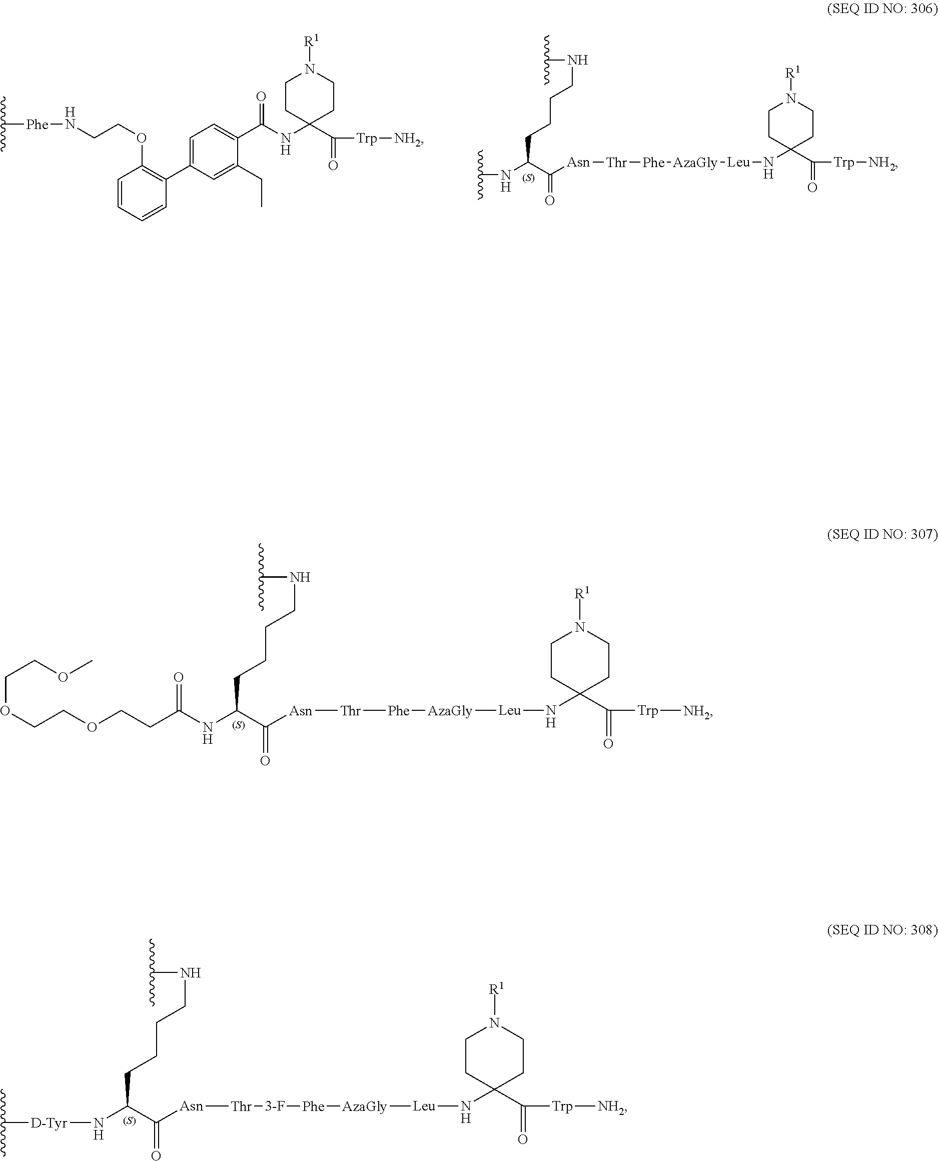

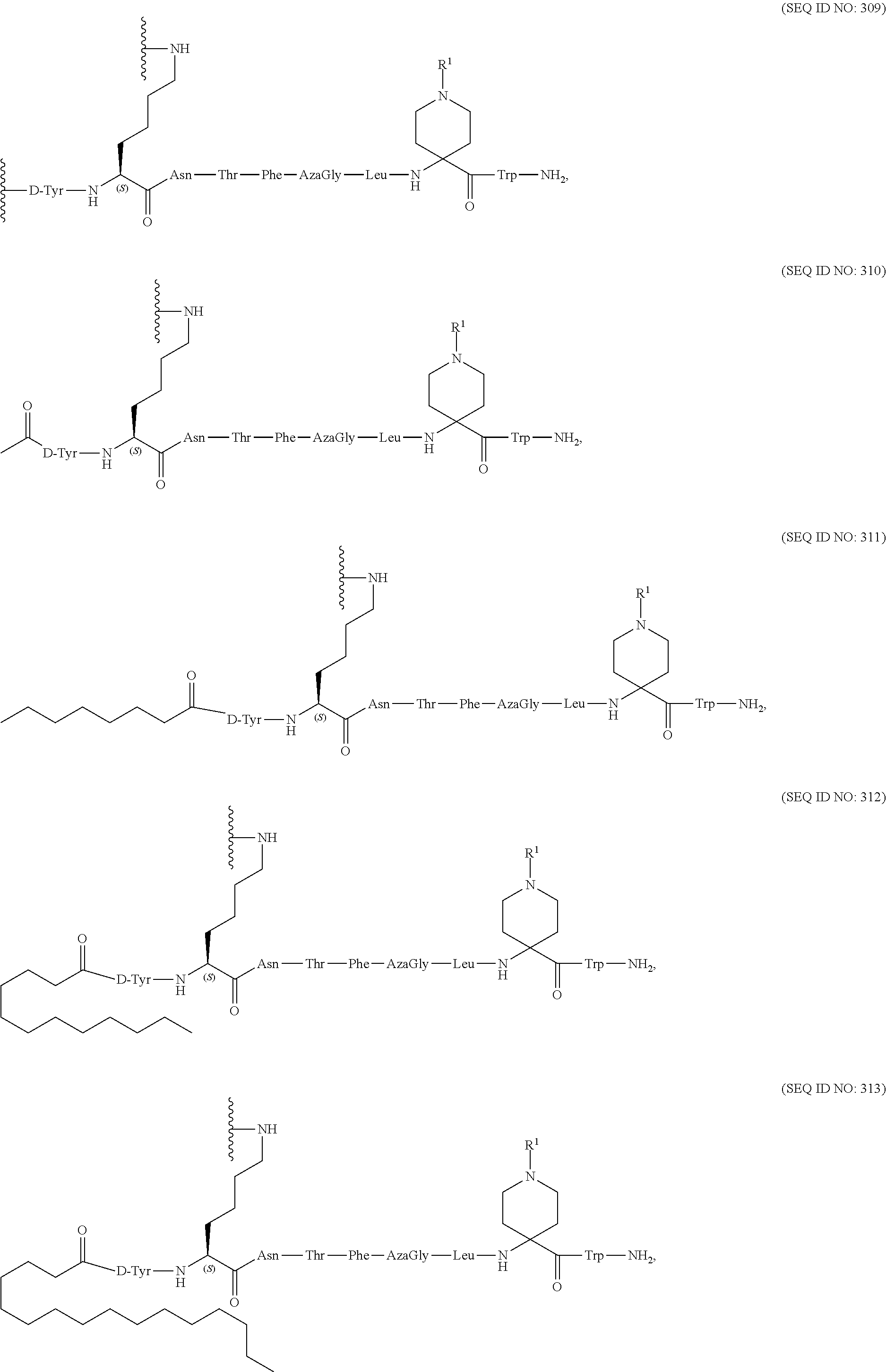

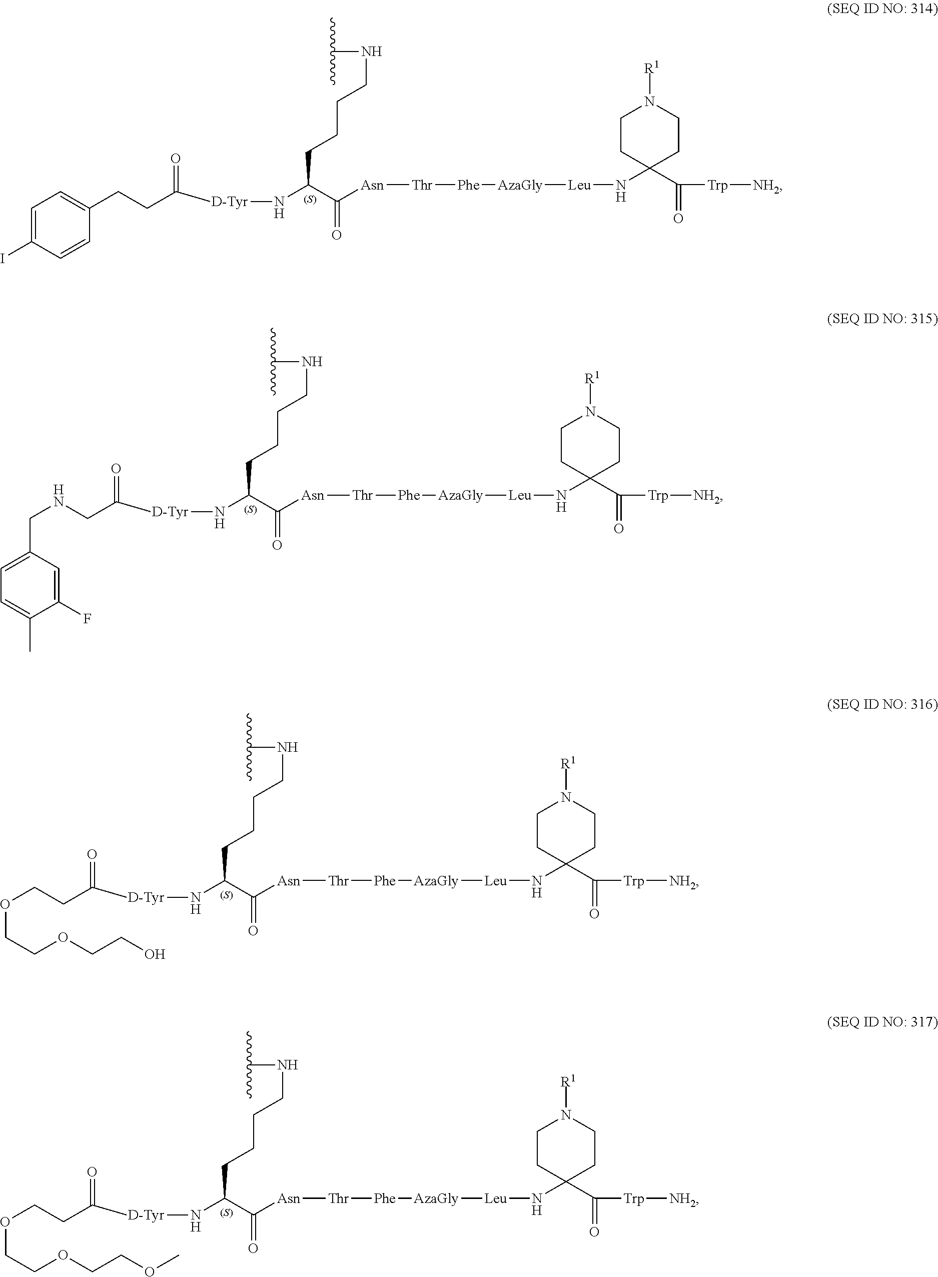

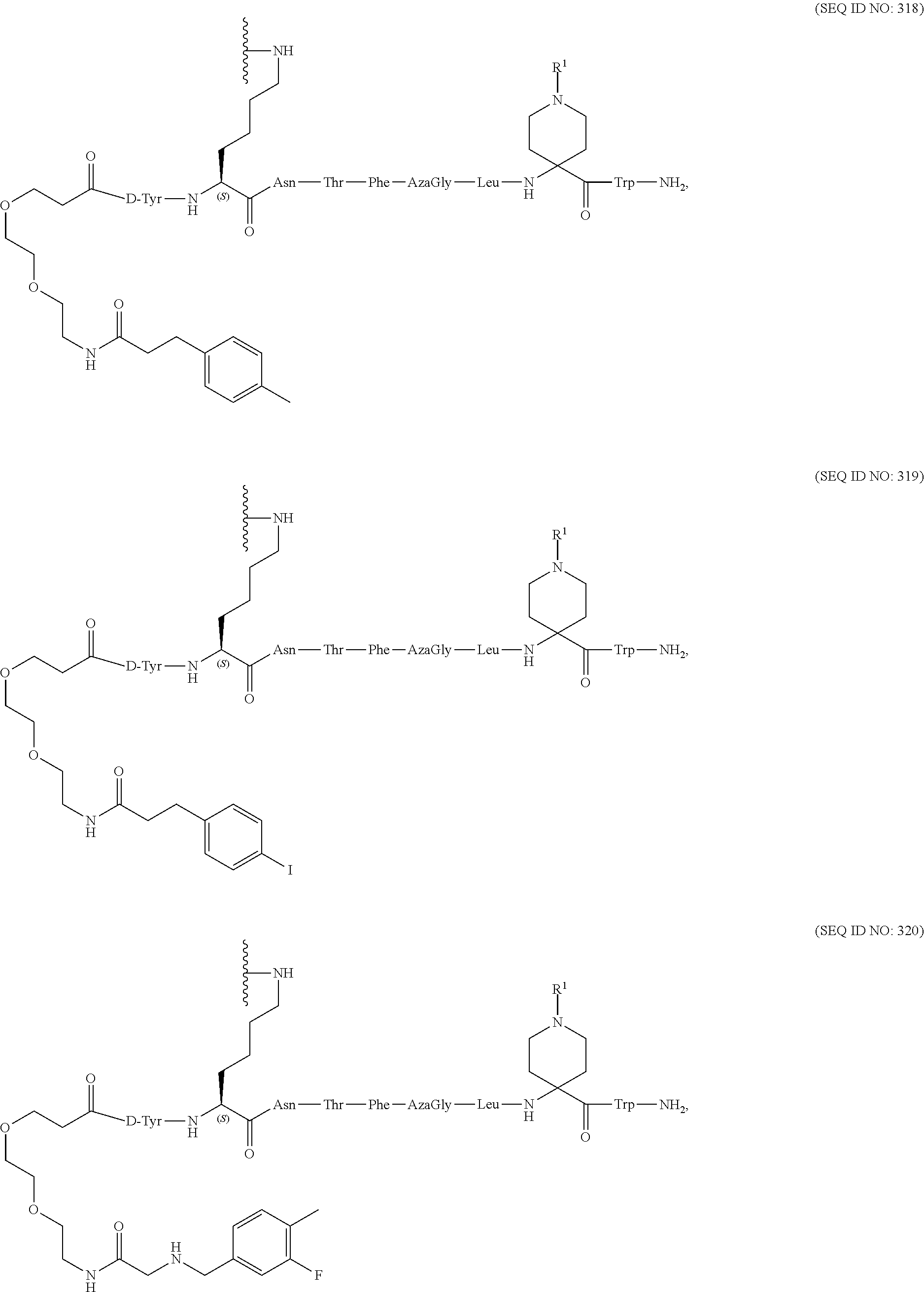

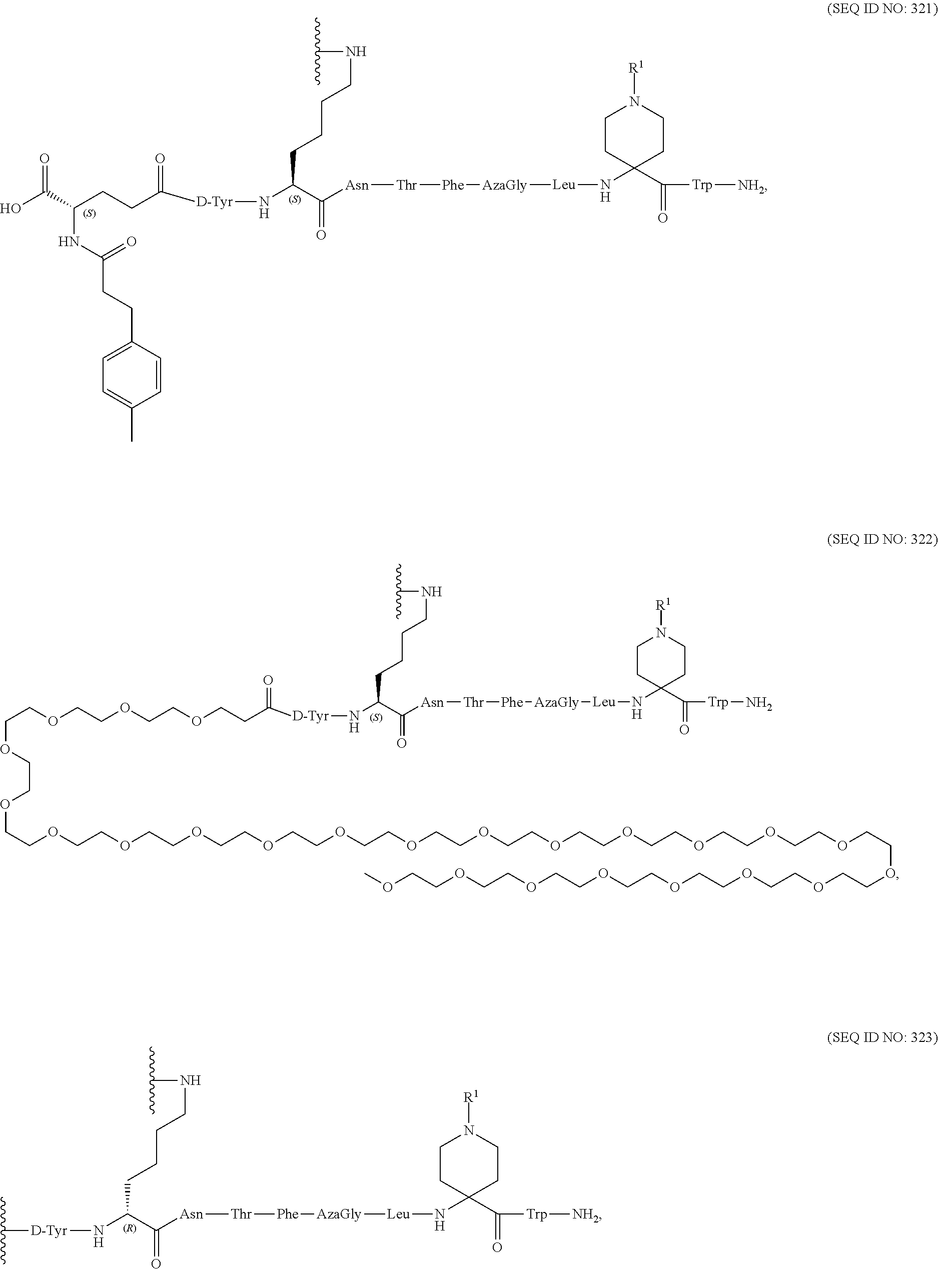

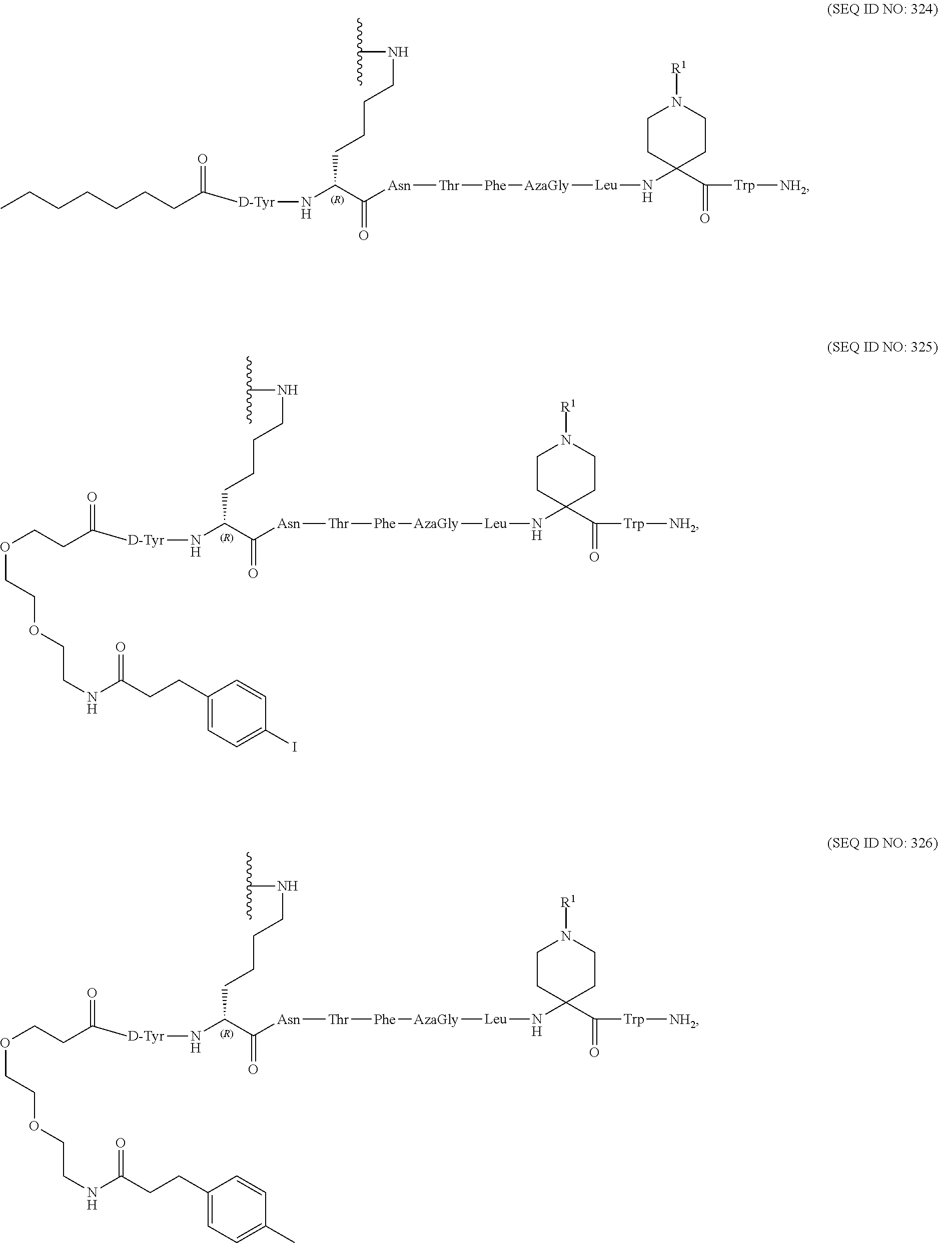

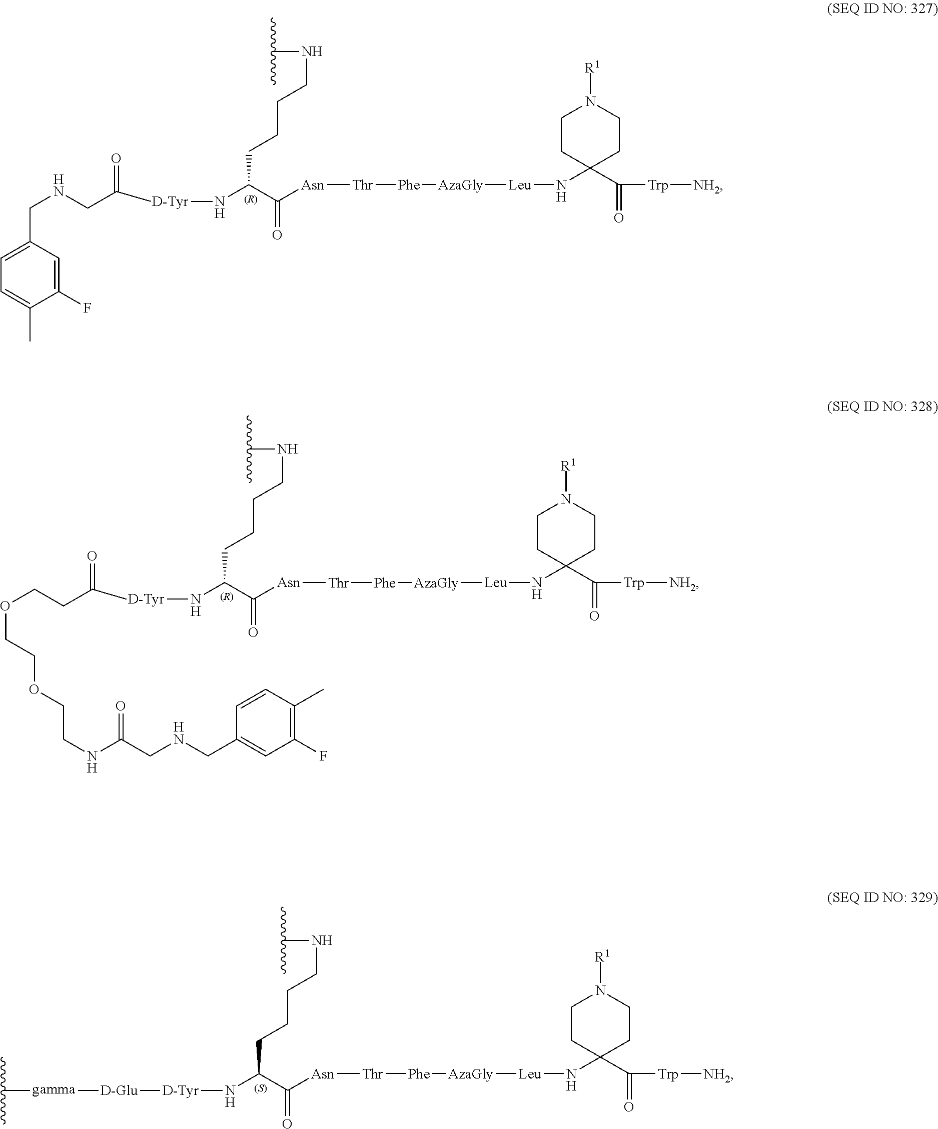

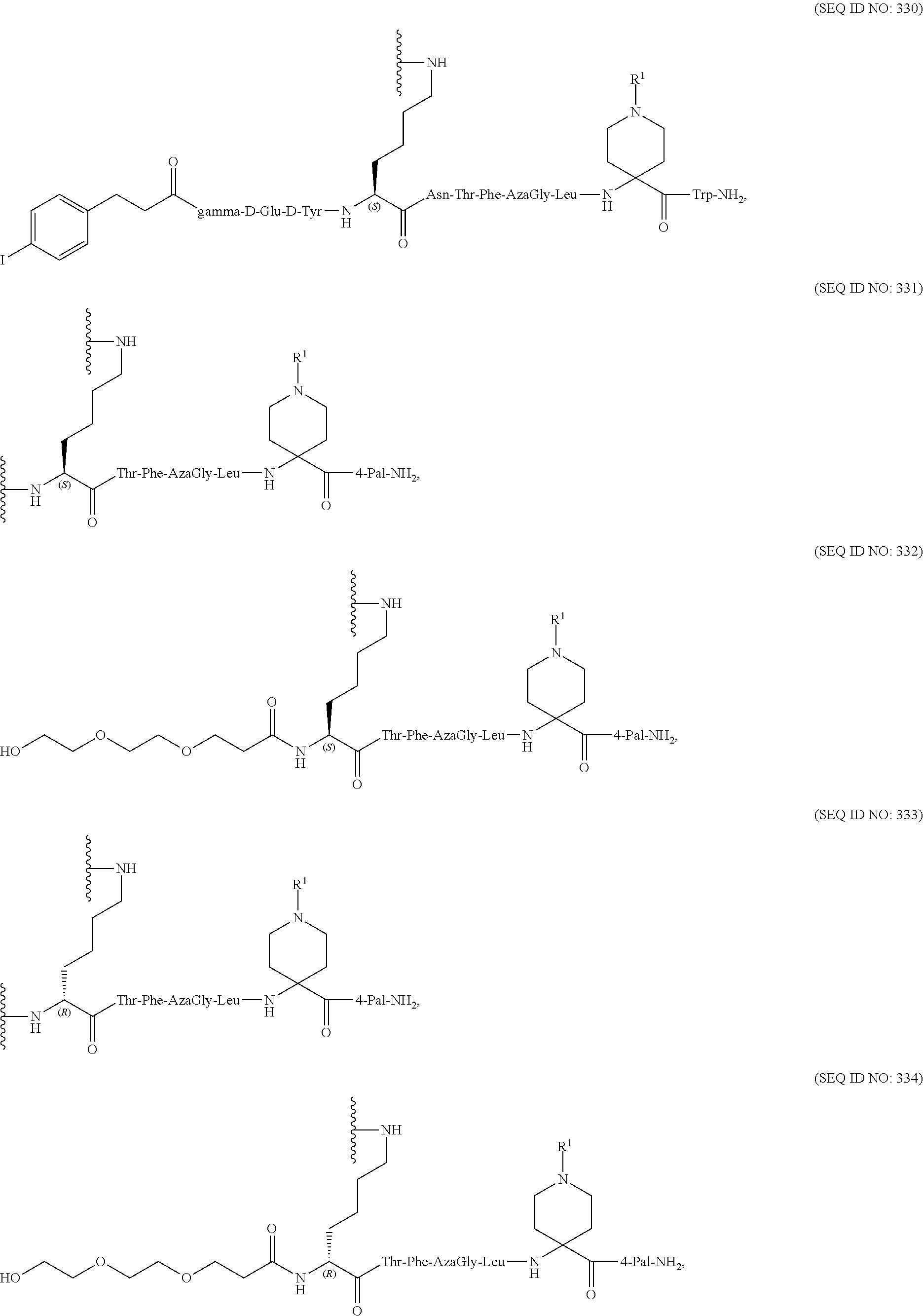

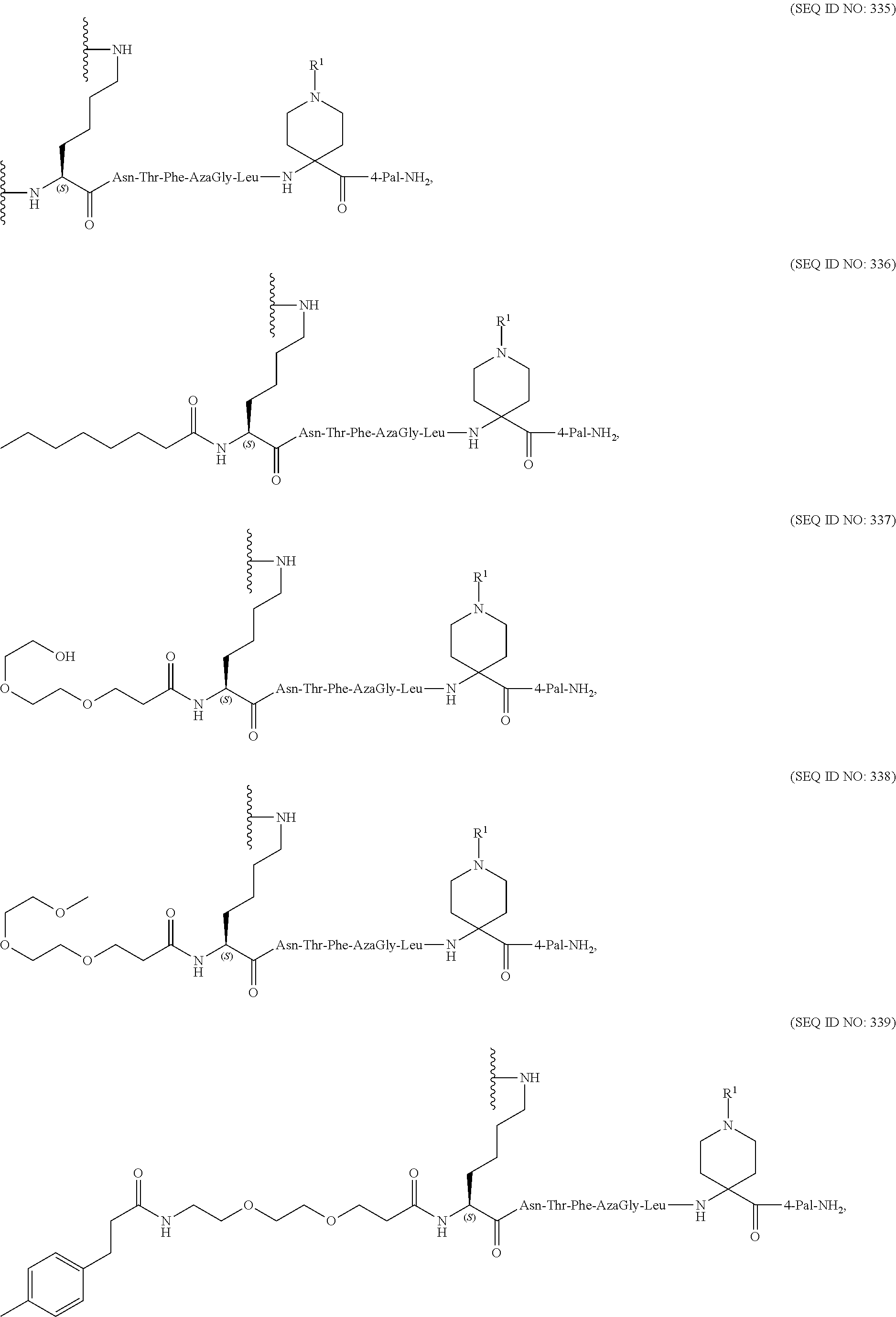

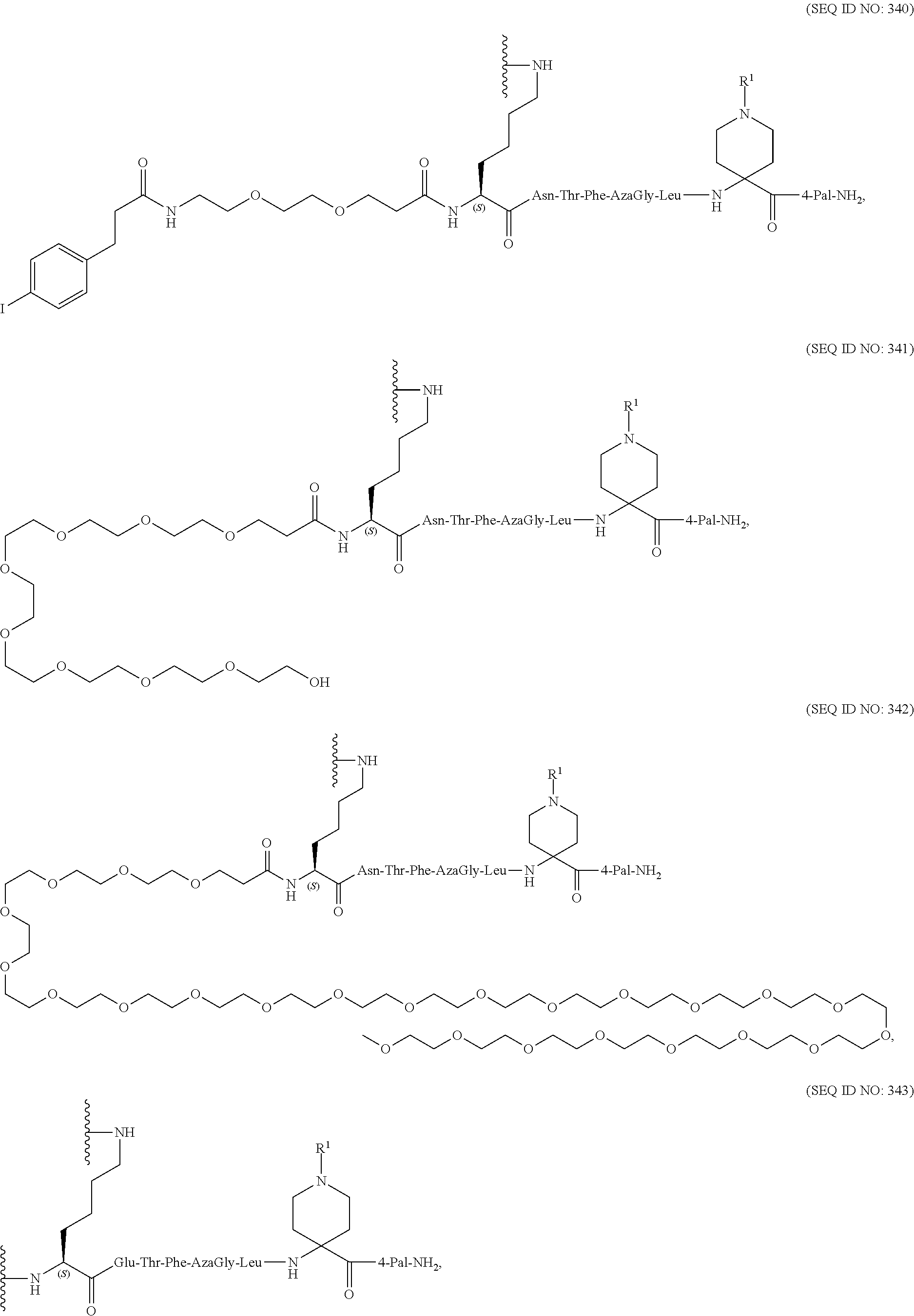

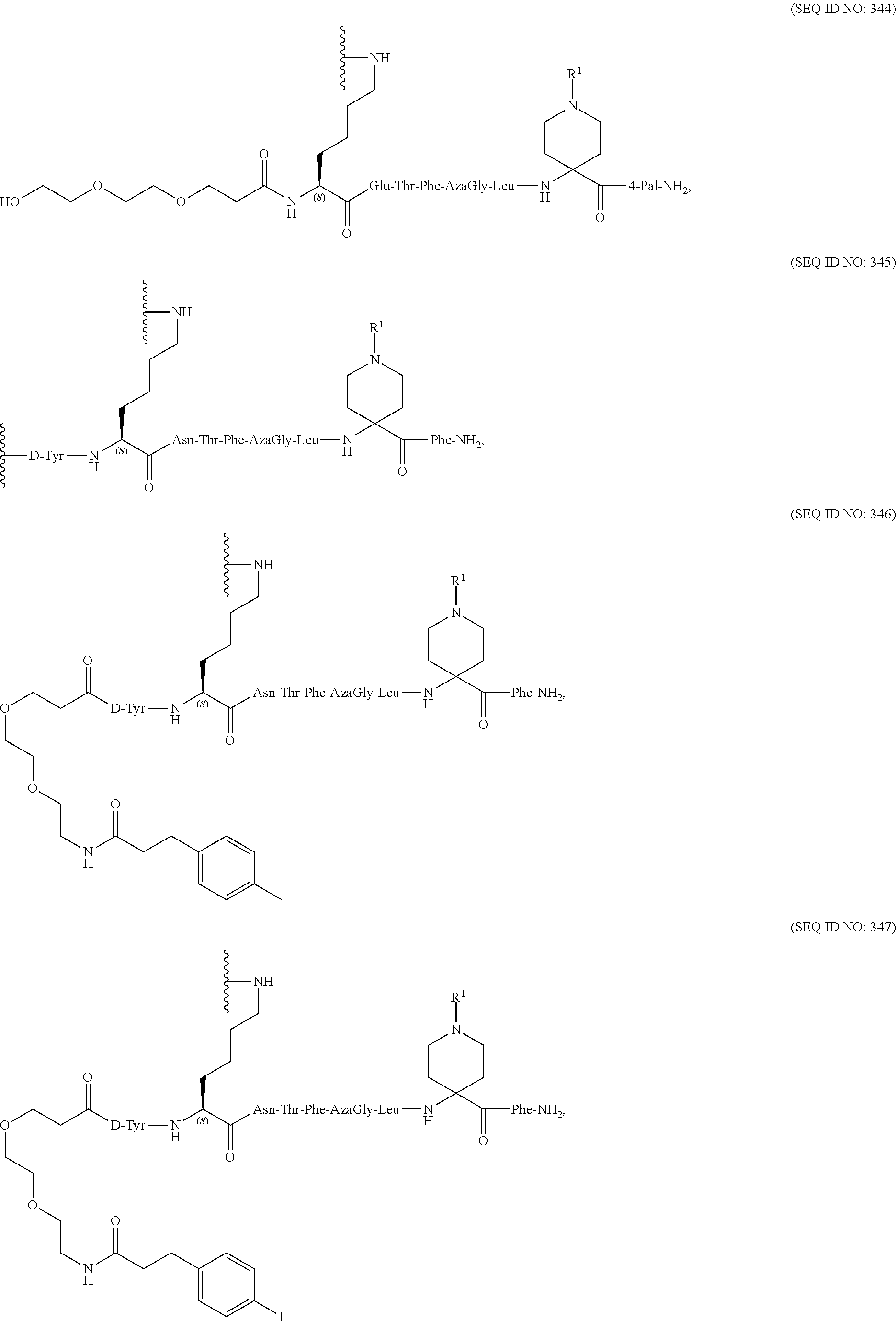

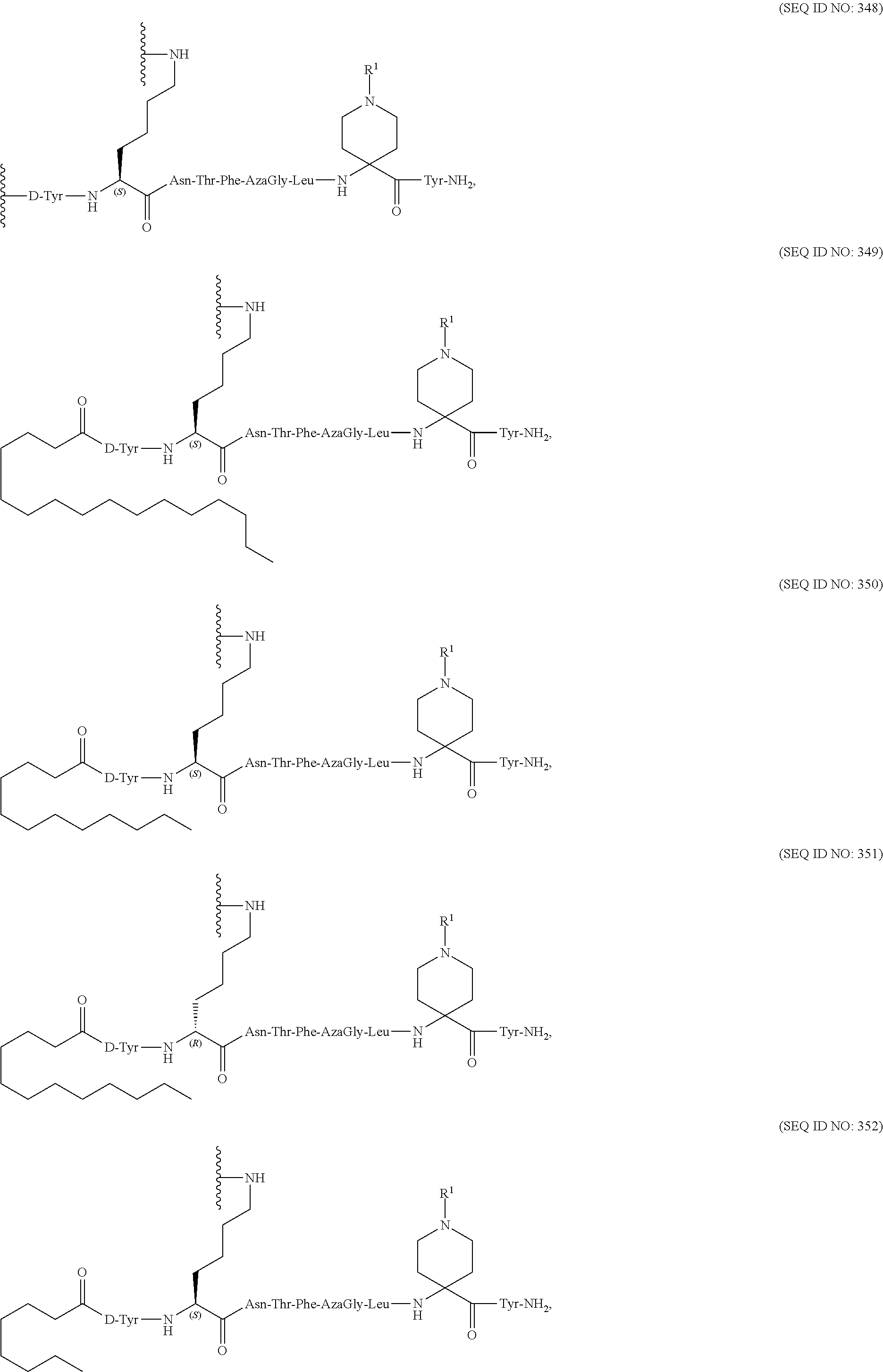

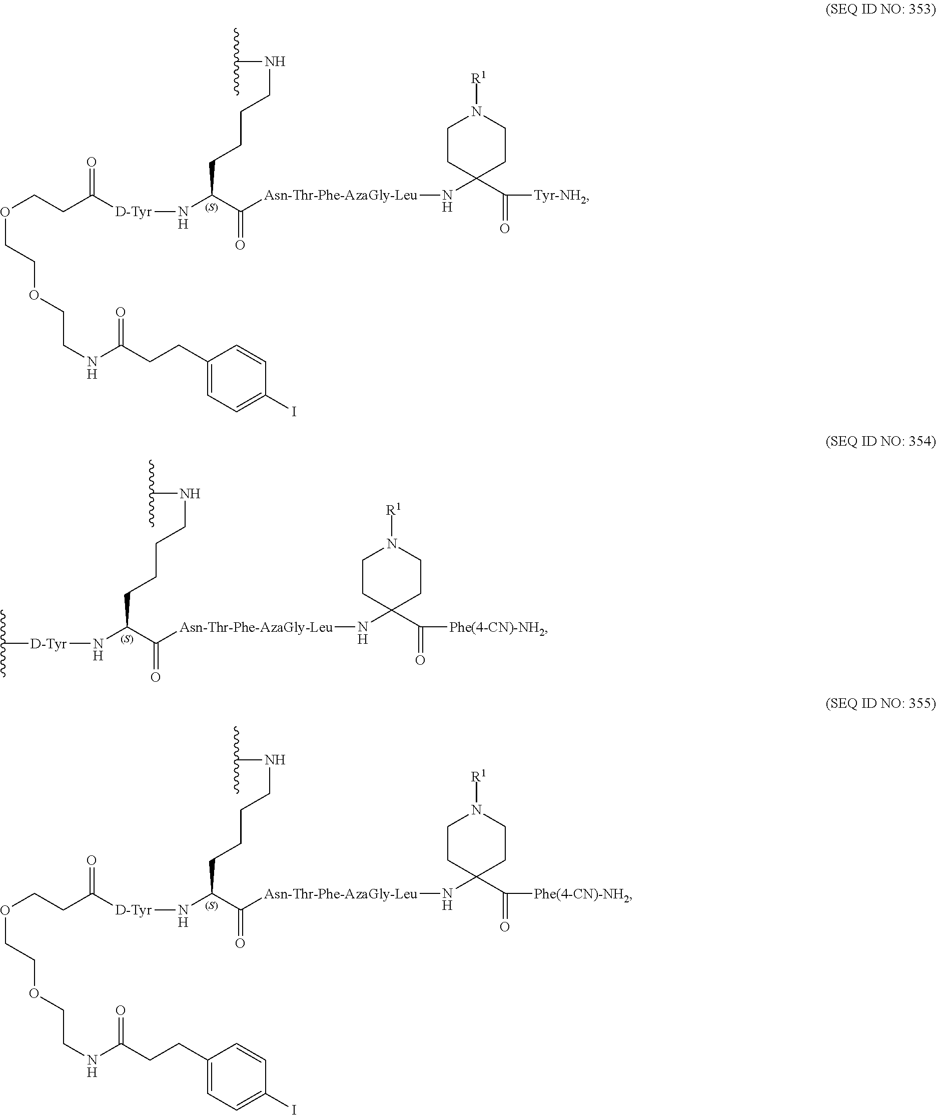

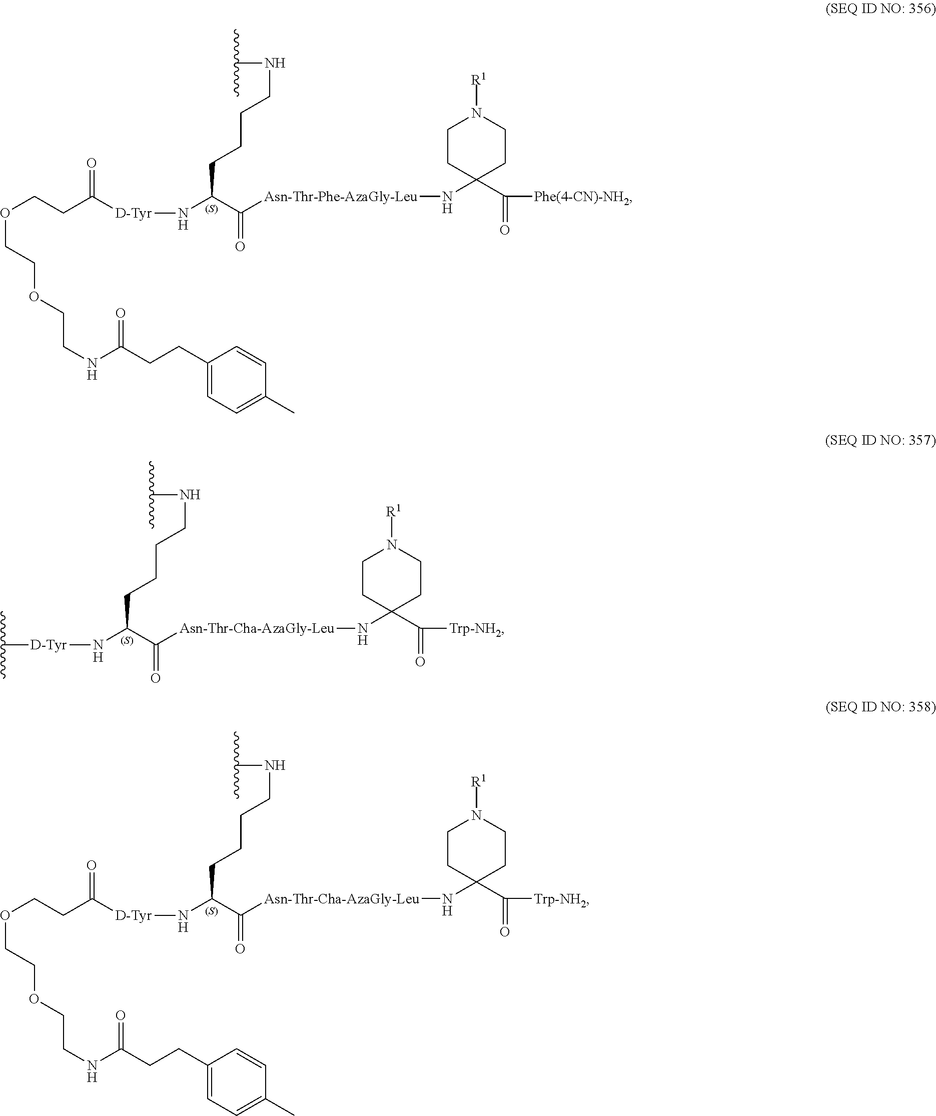

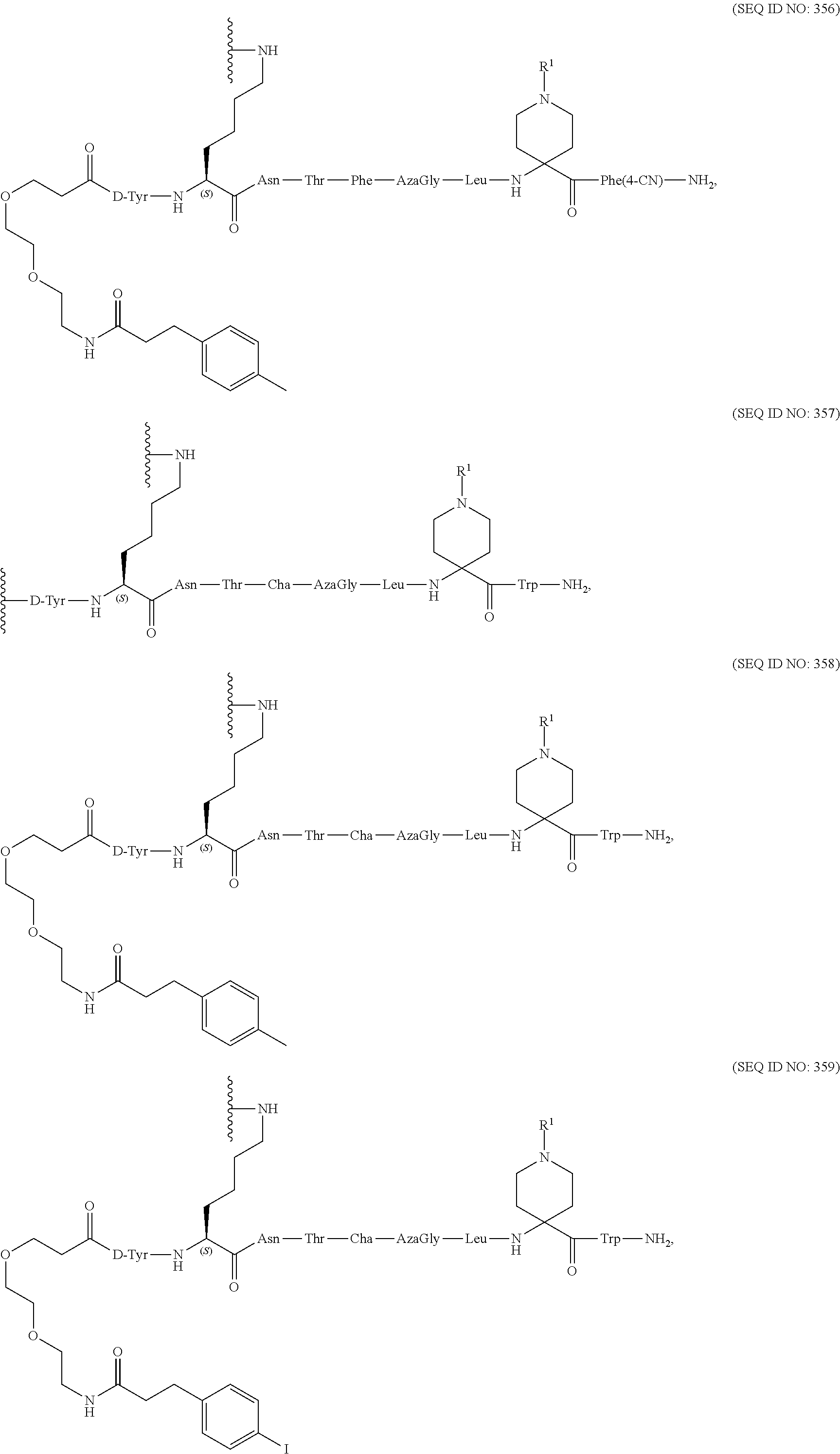

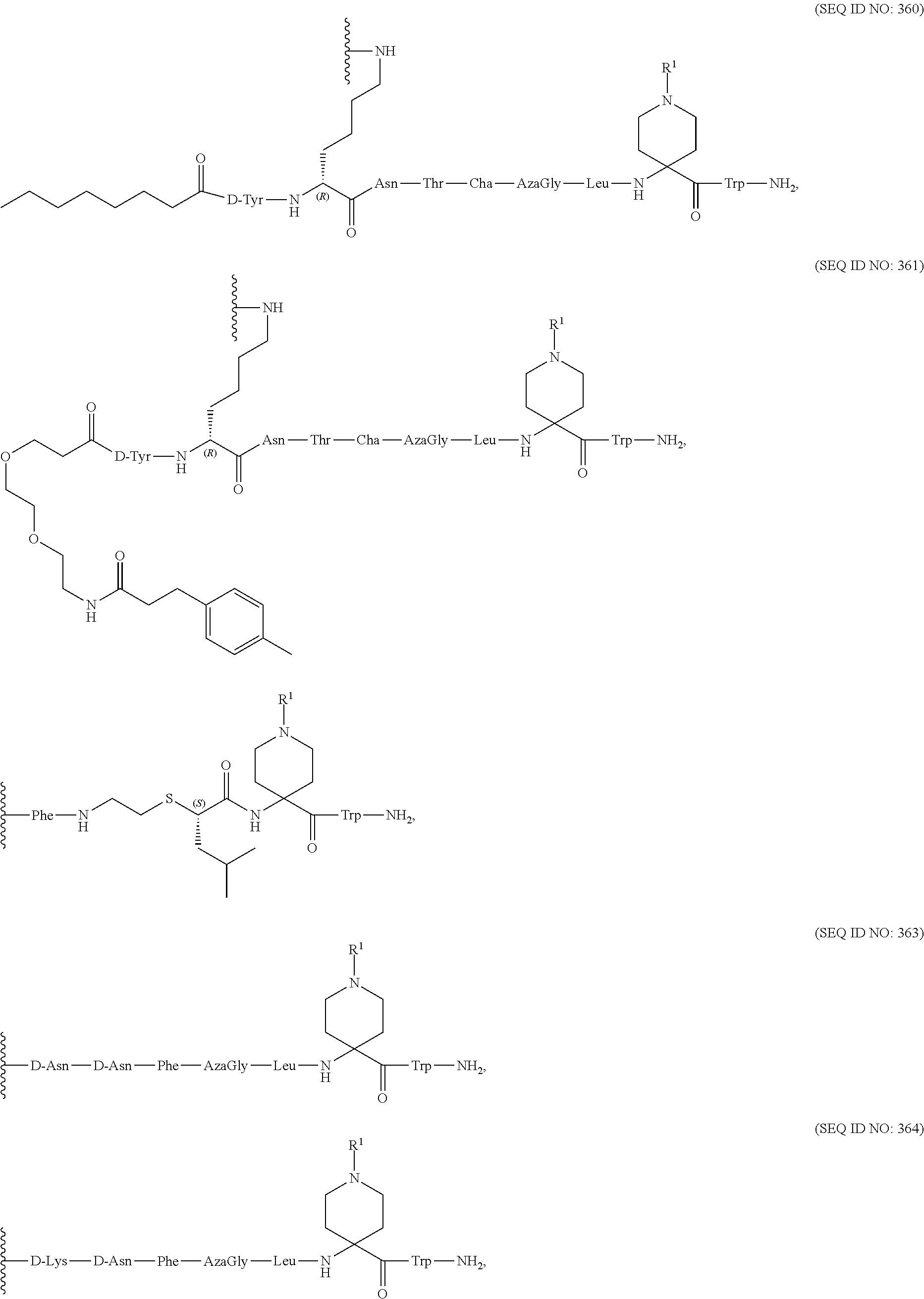

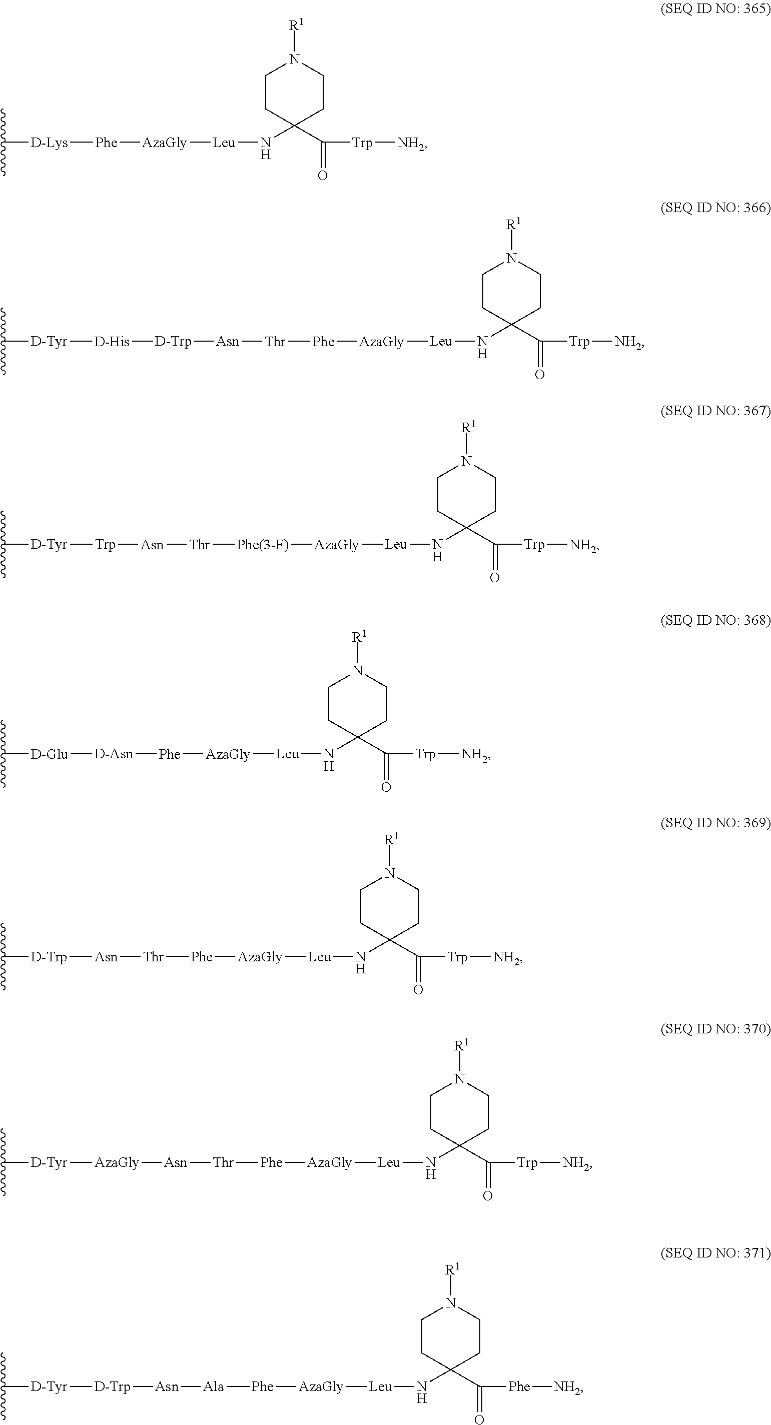

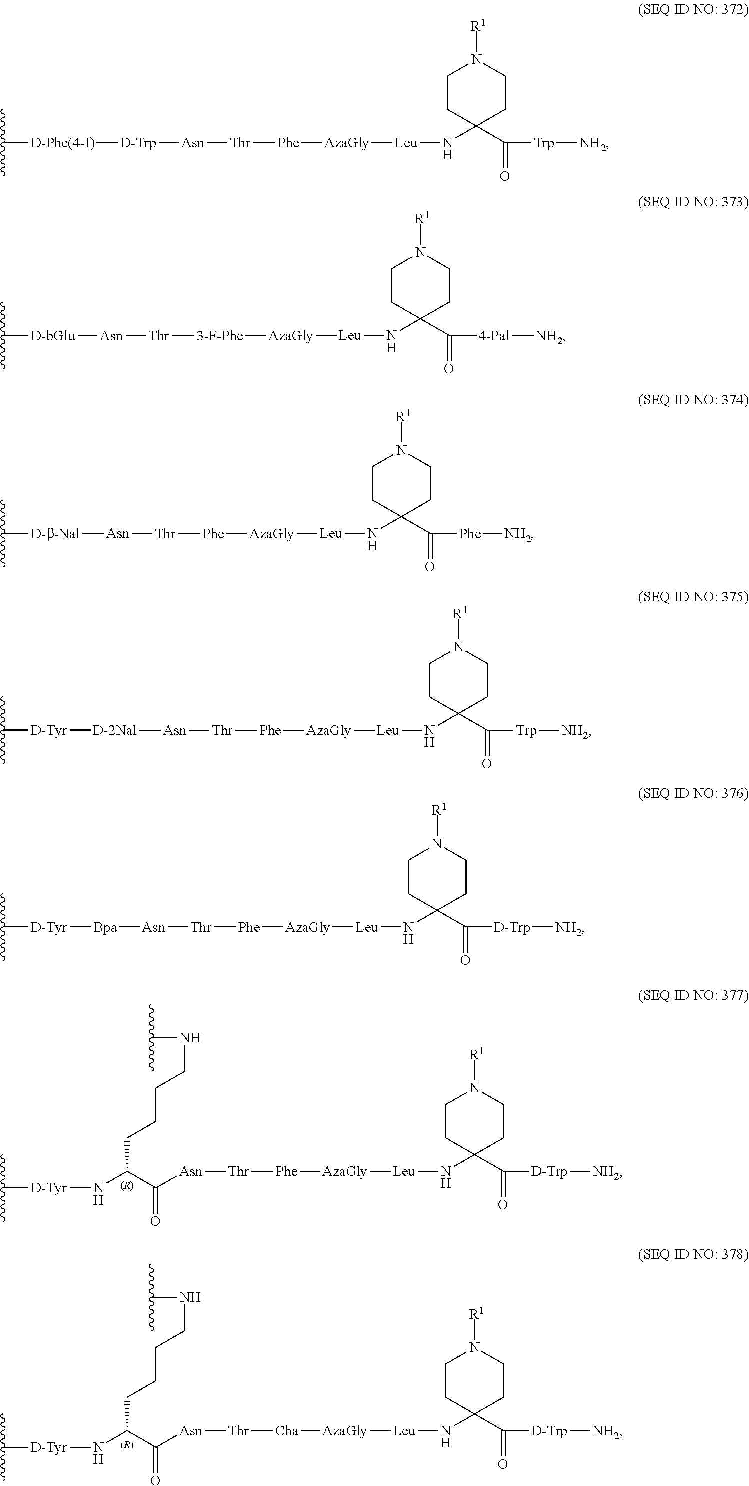

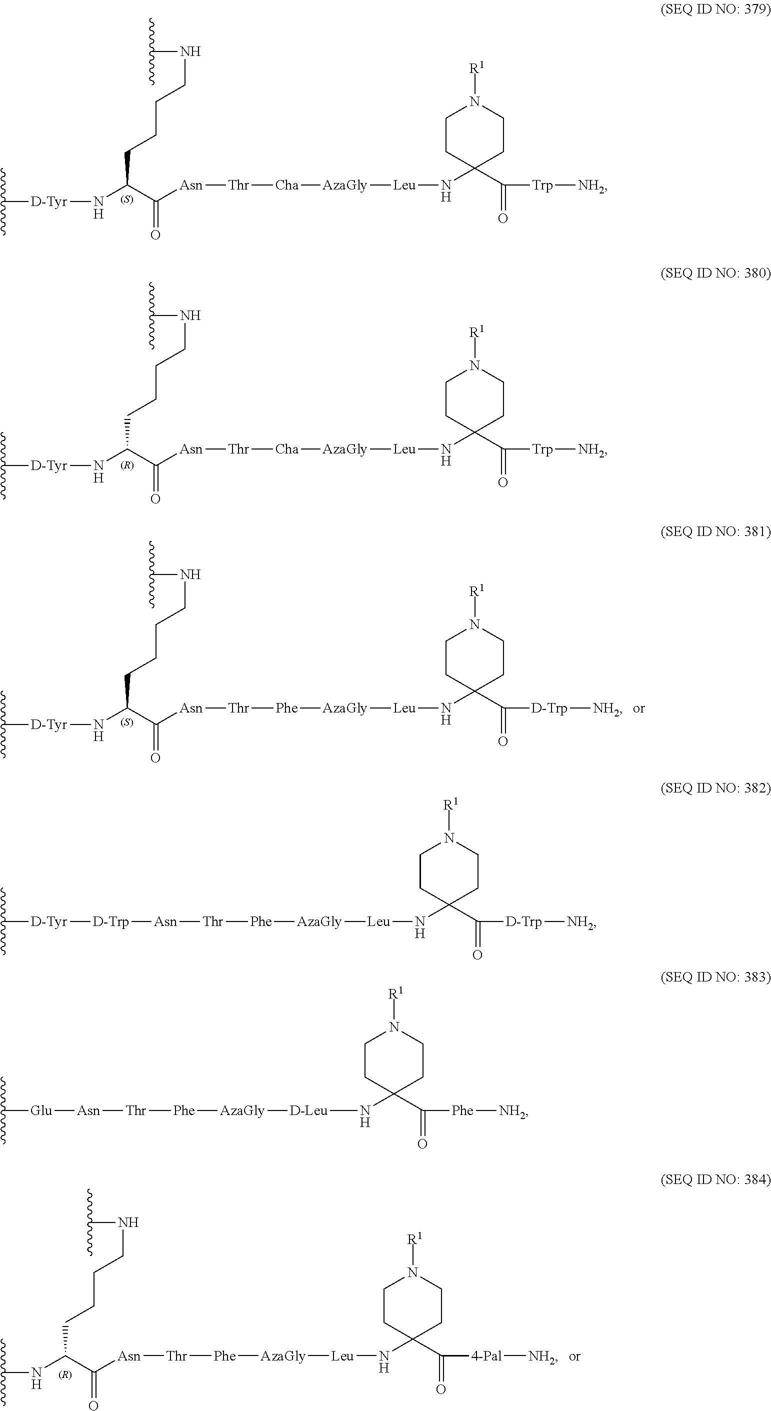

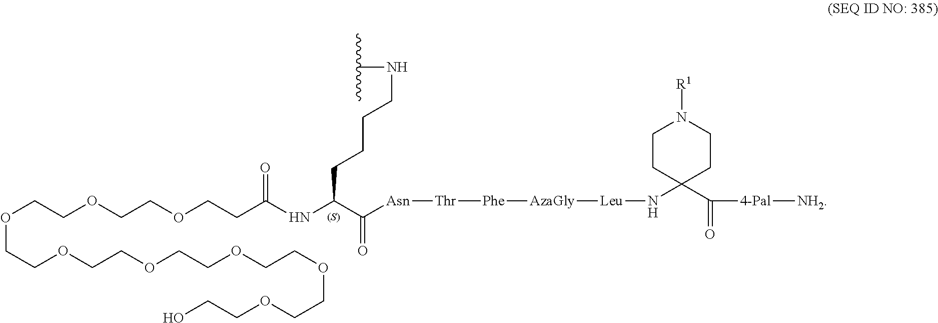

Embodiment 46. The compound of any one of embodiments 1-45, or a pharmaceutically acceptable salt thereof, wherein R^{a} is a chelating-moiety independently selected from the group consisting of:
1,4,7,10-tetraazacyclododecane-1,4,7,10-tetraacetic acid (DOTA);
2,2′,2″(10-(2-amino-2-oxoethyl)-1,4,7,10-tetraazacyclododecane-1,4,7-triyl)triacetic acid (PSC);
1,4,7,10-tetraazacyclododecane-1,4,7-triacetic acid (DO3A);
1,4,7,10-tetraazacyclododecane-1,7-diacetic acid (DO2A);
α,α′,α″,α″′-tetramethyl-1,4,7,10-tetraazacyclododecane-1,4,7,10-tetraacetic acid (DOTMA);
1,4,7,10-tetrakis(carbamoylmethyl)-1,4,7,10-tetraazacyclododecane (DOTAM);
1,4,7,10-tetraazacyclododecane-1,4,7,10-tetrapropionic acid (DOTPA);
2,2′,2″-(10-(2-amino-2-oxoethyl)-1,4,7,10-tetraazacyclododecane-1,4,7-triyl)triacetic acid:
benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA);
6,6′-(((pyridine-2,6-diylbis (methylene))bis((carboxy methyl)azanediyl))bis(methylene))dipicolinic acid (H₄pypa);
H₄pypa-benzyl;
6,6′,6″,6′″-(((pyridine-2,6-diylbis(methylene))bis(azanetriyl))tetrakis(methylene))-tetrapicolinic acid (H₄py4pa); H-₄py4pa-benzyl;
2,2′,2″-(1,4,7-triazacyclononane-1,4,7-triyl)triacetic acid (NOTA);
6,6′-((1,4,10,13-tetraoxa-7,16-diazacyclooctadecane-7,16-diyl)bis(methylene))dipicolinic acid (macropa);
2,2′,2″,2″′-(1,10-dioxa-4,7,13,16-tetraazacyclooctadecane-4,7,13,16-tetrayl)tetraacetic acid (crown);
6,6′-((ethane-1,2-diylbis((carboxymethyl)azanediyl))bis(methylene))dipicolinic acid (H₄octapa);
H₄octapa-benzyl; and
3,6,9,12-tetrakis(carboxymethyl)-3,6,9,12-tetraazatetradecanedioic acid (TTHA); or a radionuclide complex thereof.
Embodiment 47. The compound of any one of embodiments 1-45, or a pharmaceutically acceptable salt thereof, wherein R^{a} is a chelating moiety selected from the group consisting of:
1,4,7,10-tetraazacyclododecane-1,4,7,10-tetraacetic acid (DOTA) and
1,4,7,10-tetraazacyclododecane-1,4,7-triacetic acid (DO3A); or a radionuclide complex thereof.

Embodiment 48. The compound of any one of embodiments 1-45, or a pharmaceutically acceptable salt thereof, wherein R^{a} is a chelating moiety independently selected from the group consisting of:

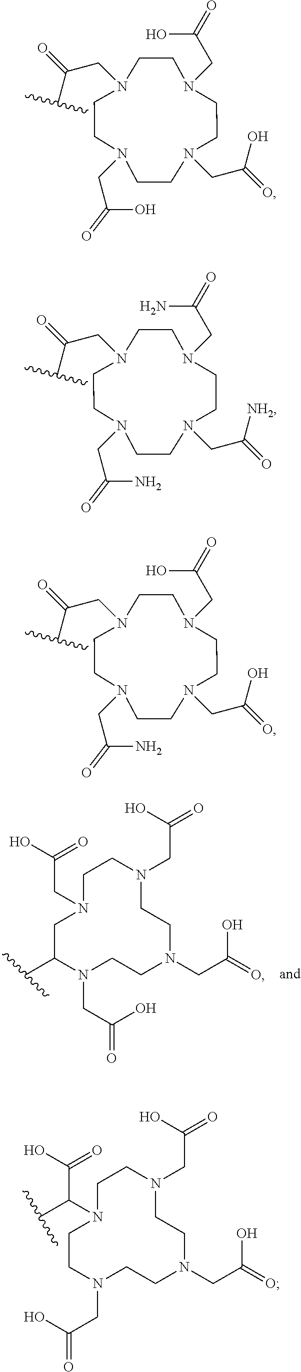 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

Embodiment 49. The compound of any one of embodiments 1-45, or a pharmaceutically acceptable salt thereof, wherein R^{a} is

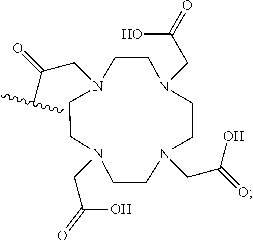 [SMILES: *C(=O)CN1CCN(CC(=O)O)CCN(CC(=O)O)CCN(CC(=O)O)CC1]

or a radionuclide complex thereof.

Embodiment 50. The compound of any one of embodiments 1-45, or a pharmaceutically acceptable salt thereof, wherein R^{a} is independently selected from:

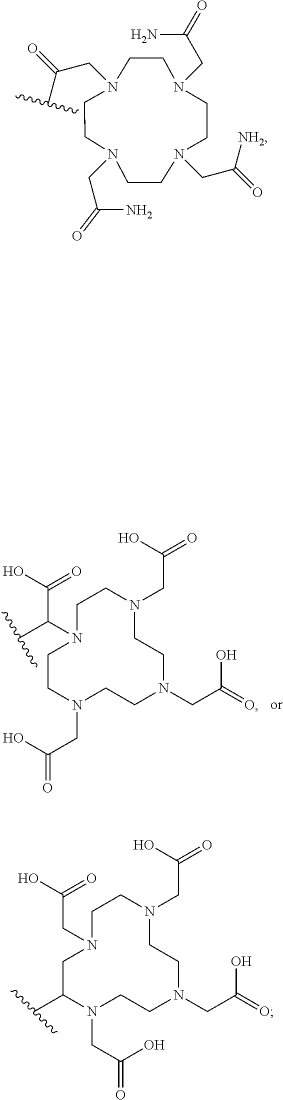 [SMILES: *C(=O)CN1CCN(CC(N)=O)CCN(CC(N)=O)CCN(CC(N)=O)CC1 ; *C(C(=O)O)N1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O]

or a radionuclide complex thereof.

Embodiment 51. The compound of any one of embodiments 1-50, or a pharmaceutically acceptable salt thereof, wherein:
-L- is absent, *-L¹-, *—NR¹⁷-L¹-, *—NR¹⁷-L⁵-L¹, *—NR¹⁷-L⁵-C(═O)-L¹, *—NR¹⁷-L⁵-NR¹⁷—C(═O)-L¹-, *-L⁵-C(═O)-L¹-, *-L⁵-L¹-, *—NR¹⁷-L⁵-NR¹⁷-L¹-, *—NR¹⁷-L⁵-C(═O)NR¹⁷-L¹-, *—NR¹⁷L⁵-C(═O)NR¹⁷-L¹-, *-(L³)-_{w}, *—NR¹⁷-L⁵-C(═O)—, or *-(L³)_{w}-NR¹⁷-L⁵-C(═O)-L¹-; wherein * denotes the attachment point to R^{a};
L⁵ is substituted or unsubstituted C₁-C₆ alkylene;
or L⁵ and R¹⁷ are taken together with the N atom to which they are attached to form N-heterocyclyl;
R¹⁷ is selected from hydrogen. C₁-C₆ alkyl, C₁-C₆ alkyl-CO₂H, —(CH₂CH₂O), —CH₂CH₂—CO₂H;
L¹ is absent, -L²-, -L²-(L³)_{w}-L², or -L²-(L³)_{w}-L²-(L³)_{w}-;
each L² is independently absent, —C₀-C₆ alkylene-(substituted or unsubstituted aryl)-C₀-C₆ alkylene-C(═O)—, —C₀-C₆ alkylene-(substituted or L unsubstituted arylene)-C₀-C₆ alkylene-OC(═O)—, —C₀-C₆ alkylene-(substituted or unsubstituted cyclohexylene)-C₀-C₆ alkylene-C(═O)—, —C₀-C₆ alkylene-(substituted or unsubstituted heterocycloalkylene)-C₀-C₆ alkylene-C(═O)—, —C₀-C₆ alkylene-(substituted or unsubstituted heteroarylene)-C₀-C₆ alkylene-C(═O)—, —C₀-C₆ alkylene-(substituted or unsubstituted heteroarylene)-C₀-C₆alkylene-OC(═O)—, —C₁-C₂₀ polyethylene glycol, —C₄-C₂₀ polyethylene glycol-C(═O)—, substituted or unsubstituted —C₁-C₂₀ alkylene, substituted or unsubstituted —C₁-C₂₀ alkylene-C(═O)—, substituted or unsubstituted 2 to 20 membered heteroalkylene, —(CH₂CH₂O)—CH₂—, —(CH₂CH₂O), —CH₂CH₂—, —(CH₂CH₂O)_{z}—CH₂—C(═O)—, or —(CH₂CH₂O)—CH₂CH₂—C(═O)—;
each z is independently 1, 2, 3, 4, 5, or 6;
each L³ is independently selected from natural or unnatural amino acids, wherein any free anine of an amino acid or peptide bond is optionally independently substituted with U, and wherein when two or more amino acids are present then the N atom of the amide linking the amino acids is optionally substituted with —CH₃;
each L⁴ is independently selected from C₁-C₆ alkylene, C(═)—C₁-C₆ alkylene-C(═O), C(═O)—NH—C₁-C₆ alkylene-C(═O), C(═O)—C₁-C₆ alkylene-(substituted or unsubstituted heteroarylene)-C₁-C₆ alkylene-C(═O), C₁-C₆ alkylene-C(═O), —C(═O)—C₁-C₆ alkylene-(substituted or unsubstituted arylene), and C₁-C₆ alkylene-(substituted or unsubstituted arylene)-C(═O), wherein if L⁴ is present then: L⁴ is attached to the any one of X¹, X², X³, X⁴, X⁵, X⁶, or X⁷, or L⁴ is attached to X⁸ if X¹, X², X³, X⁴, X⁵, X⁶, and X⁷ are absent; and
each w is independently 1, 2, 3, 4, 5, or 6.

Embodiment 52. The compound of embodiment 51, or a pharmaceutically acceptable salt thereof, wherein:

each L² is independently absent, -(substituted or unsubstituted phenylene)-C₀-C₆ alkylene-C(═O)—, -(substituted or unsubstituted cyclohexylene)-C₀-C₆ alkylene-C(═O)—, -(substituted or unsubstituted heterocycloalkylene)-C₀-C₆ alkylene-C(═O)—, -(substituted or unsubstituted heteroarylene)-C₀-C₆ alkylene-C(═O)—, substituted or unsubstituted C₁-C₂₀ alkylene-C(═O)—, —(CH₂CH₂O)_{z}—CH₂—, —(CH₂CH₂CH₂O)—CH₂CH₂—, —(CH₂CH₂O)_{z}—CH₂—C(═O)—, or —(CH₂CH₂O)—CH₂CH₂—C(═O)—; and
each z is independently 1, 2, 3, 4, 5, or 6.

Embodiment 53. The compound of embodiment 51, or a pharmaceutically acceptable salt thereof, wherein:
each L² is independently absent, -(substituted or unsubstituted phenylene)-C₀-C₆ alkylene-C(═O)—, -(substituted or unsubstituted cyclohexylene)-C₀-C₆ alkylene-C(═O)—, -(substituted or unsubstituted heterocycloalkylene)-C₀-C₆ alkylene-C(═O)—, -(substituted or unsubstituted heteroarylene)-C₀-C₆ alkylene-C(═O)—, substituted or unsubstituted C₁-C₂₀ alkylene-C(═O)—, —(CH₂CH₂O)_{z}—CH₂—C(═O)—, or —(CH₂CH₂O)_{z}—CH₂CH₂—C(═O)—; and
each z is independently 1, 2, 3, 4, 5, or 6.

Embodiment 54. The compound of any one of embodiments 51-53, or a pharmaceutically acceptable salt thereof, wherein:
L- is *—NR¹⁷—, *—NR¹⁷-L⁵, *—NR⁵-L⁵-C(═O)—, *—NR¹⁷—C₀-C₆ alkylene-(substituted or unsubstituted phenylene)-C₀-C₆alkylene-C(═O)—, *—NR¹⁷—C₀-C₆, alkylene-(substituted or unsubstituted cyclohexylene)-C₀-C₆ alkylene-C(═O)—, *—NR¹⁷—C₀-C₆ alkylene-(substituted or unsubstituted heterocycloalkylene)-C₀-C₆ alkylene-C(═O)—, *—NR¹⁷—C₀-C₆ alkylene-(substituted or unsubstituted heteroarylene)-C₀-C₆ alkylene-C(═O)—, *—NR¹⁷—C₀-C₆ alkylene substituted or unsubstituted C₀-C₁₀ alkylene-C(═O)—, *—NR¹⁷—(CH₂CH₂O)_{z}—CH₂—C(═O)—, *—NR¹⁷—(CH₂CH₂O)_{z}—CH₂CH₂—C(═O)—, *— R¹⁷-L⁵-C(═O)-(L³)_{w}-, *—NR⁵-L⁵—C(═O)NR¹⁷—C₀-C₆ alkylene-(substituted or unsubstituted phenylene)-C₀-C₆ alkylene-C(═O)—, *—NR⁵-L⁵-C(═O)NR¹⁷—C₀-C₆ alkylene-(substituted or unsubstituted cyclohexylene)-C₀-C₆ alkylene-C(═O)—, *—NR⁵-L⁵-C(═O)NR—C₀-C₆ alkylene-(substituted or unsubstituted heterocycloalkylene)-C₀-C₆ alkylene-C(═O)—, or *—NR⁵-L⁵-C(═O)NR¹⁷—C₀-C₆ alkylene-(substituted or unsubstituted heteroarylene)-C₀-C₆ alkylene-C(═O)—; and
z is 1, 2, 3, 4, 5, or 6;
wherein * denotes the attachment point to R^{a}.

Embodiment 55. The compound of any one of embodiments 51-53, or a pharmaceutically acceptable salt thereof, wherein:
each P is independently selected from the group consisting of alanine (Ala), arginine (Arg), asparagine (Asn), aspartate (Asp), glutamine (Gln), glutamate (Glu), glycine (Gly), leucine (Leu), lysine (Lys), 3-(2-naphthyl)-L-alanine (2-Nal), 3-(4-pyridyl)alanine (4-Pal), phenylalanine (Phe), serine (Ser), sarcosine, tyrosine (Tyr), 3-sulfo-alanine(Ala-SO₃H), methionine (Met), valine (Val), 2-(3-aminopropoxy)-[1,1-biphenyl]-4-carboxylic acid, 2′-(3-aminopropoxy)-[1,1′-biphenyl]-4-carboxylic acid, O-(dihydroxy(oxo)-16-phosphaneyl)-L-serine, (S)-2-amino-4-(2H-tetrazol-5-yl)butanoic acid, and (S)-2-amino-3-(anthracen-9-yl)propanoic acid, wherein any free amine of an amino acid or peptide bond is optionally independently substituted with L⁴, and wherein when two or more amino acids are present then the N atom of the amide linking the amino acids is optionally substituted with —CH₃.

Embodiment 56. The compound of any one of embodiments 51-53, or a pharmaceutically acceptable salt thereof, wherein:
each L³ is independently selected from the group consisting of alanine (Ala), glycine (Gly), serine (Ser), sarcosine, methionine (Met), 3-sulfo-alanine (Ala-SO₃H), and valine (Val), wherein any free amine of an amino acid or peptide bond is optionally independently substituted with L⁴, wherein L⁴ is —C(═O)—C₁-C₆ alkylene-C(═O)— or —C(═O)—NH—C₁-C₆ alkyl-C(═O)—, and wherein when two or more amino acids are present then the N atom of the amide linking the amino acids is optionally substituted with —CH₃.

Embodiment 57. The compound of any one of embodiments 51-53, or a pharmaceutically acceptable salt thereof, wherein -(L³)_{w}- is sarcosine, sarcosine-sarcosine, sarcosine-sarcosine-sarcosine, sarcosine-sarcosine-sarcosine-sarcosine, sarcosine-sarcosine-sarcosine-sarcosine-sarcosine, sarcosine-sarcosine-sarcosine-sarcosine-sarcosine-sarcosine, valine-citrulline, valine-alanine, methionine-valine-lysine, glycine-phenylalanine-glycine-glycine, tyrosine-arginine-valine, arginine-valine, 3-sulfo-alanine, 3-sulfo-alanine-3-sulfo-alanine, 3-sulfo-alanine-3-sulfo-alanine-3-sulfo-alanine, glycine-glutamate, gly % cine-glutamate-glycine, glycine-glutamate-glutamate, methionine-tryptophan-lysine, methionine-phenylalanine-lysine, methionine-valine, methionine-valine-lysine, or phenylalanine-lysine, wherein the free amine of lysine is optionally substituted with L⁴; L⁴ is —C(═O)(CH₂)₃—C(═O)—, —C(═O)—(CH₂)₄—C(═O)—, —C(═O)—(CH₂)₅—C(═O)—, —C(═O)—(CH₂)₆—C(═O)—, —C(═O)NH—(CH₂)₃—C(═O), —C(═O)NH—(CH₂)₄—C(═O)—, —C(═O)NH—(CH₂)₅—C(═O)—, —C(═O)NH—(CH₂)₆—C(═O)—, —C(═O)—(CH₂)₂-(triazolylene)-(CH₂)₁—C(═O)— or —C(═O)—(CH₂)₂-(triazolylene)-(CH₂)₂—C(═O)—; wherein if L⁴ is present then: L⁴ is attached to the any one of X¹, X², X³, X⁴, X⁵, X⁶, or X⁷, or L⁴ is attached to X⁸ if X¹, X², X³, X⁴, X⁵, X⁶, and N⁷ are absent.

Embodiment 58. The compound of any one of embodiments 1-50, or a pharmaceutically acceptable salt thereof, wherein: -L- is: absent,

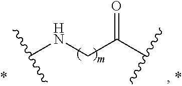

m is 1, 2, 3, 4, 5, or 6;
z is 1, 2, 3, 4, 5, or 6;
w is 1, 2,3, 4, 5, or 6;
wherein * denotes the attachment point to R^{a}.

Embodiment 59. The compound of any one of embodiments 1-50, or a pharmaceutically acceptable salt thereof, wherein: -L- is: absent,

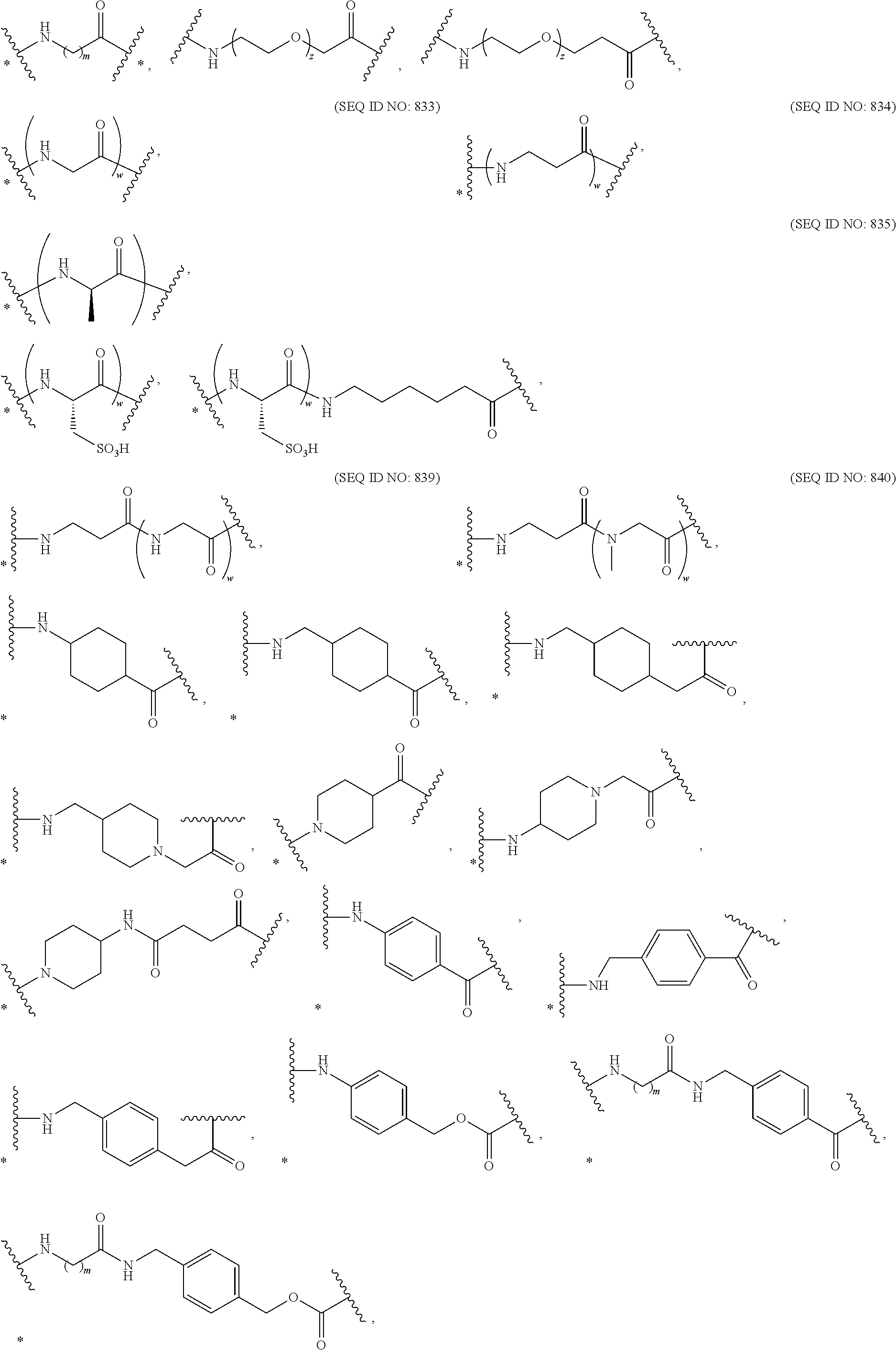

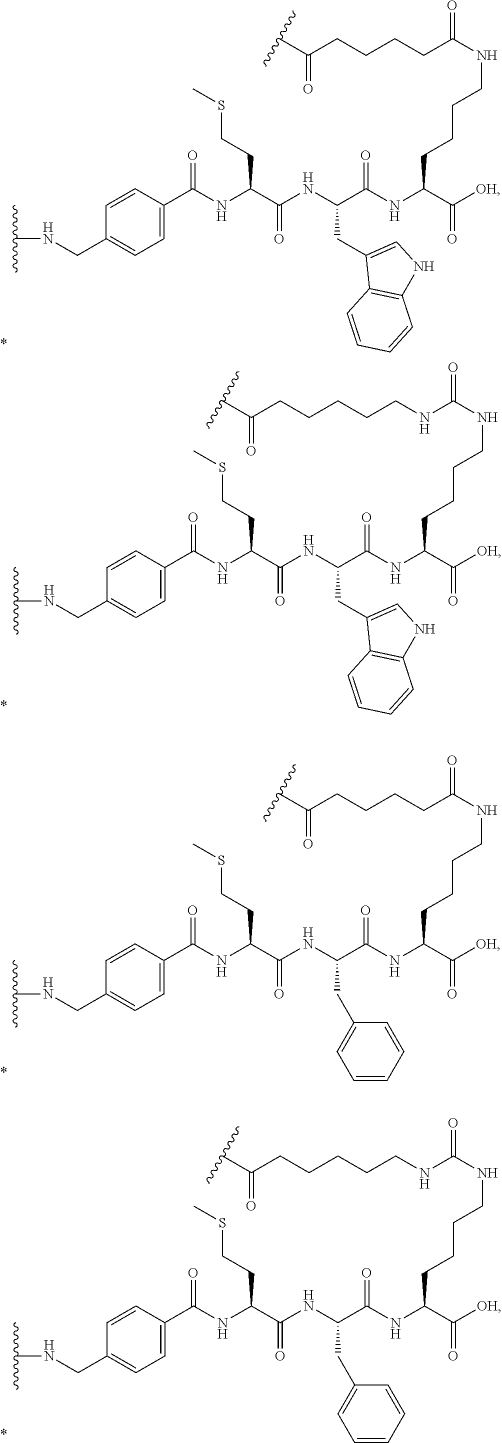 [SMILES: *NCc1ccc(C(=O)N[C@@H](CCSC)C(=O)N[C@@H](Cc2c[nH]c3ccccc23)C(=O)N[C@@H](CCCCNC(=O)CCCCC(*)=O)C(=O)O)cc1 ; *NCc1ccc(C(=O)N[C@@H](CCSC)C(=O)N[C@@H](Cc2c[nH]c3ccccc23)C(=O)N[C@@H](CCCCNC(=O)NCCCCCC(*)=O)C(=O)O)cc1 ; *NCc1ccc(C(=O)N[C@@H](CCSC)C(=O)N[C@@H](Cc2ccccc2)C(=O)N[C@@H](CCCCNC(=O)CCCCC(*)=O)C(=O)O)cc1 ; *NCc1ccc(C(=O)N[C@@H](CCSC)C(=O)N[C@@H](Cc2ccccc2)C(=O)N[C@@H](CCCCNC(=O)NCCCCCC(*)=O)C(=O)O)cc1]

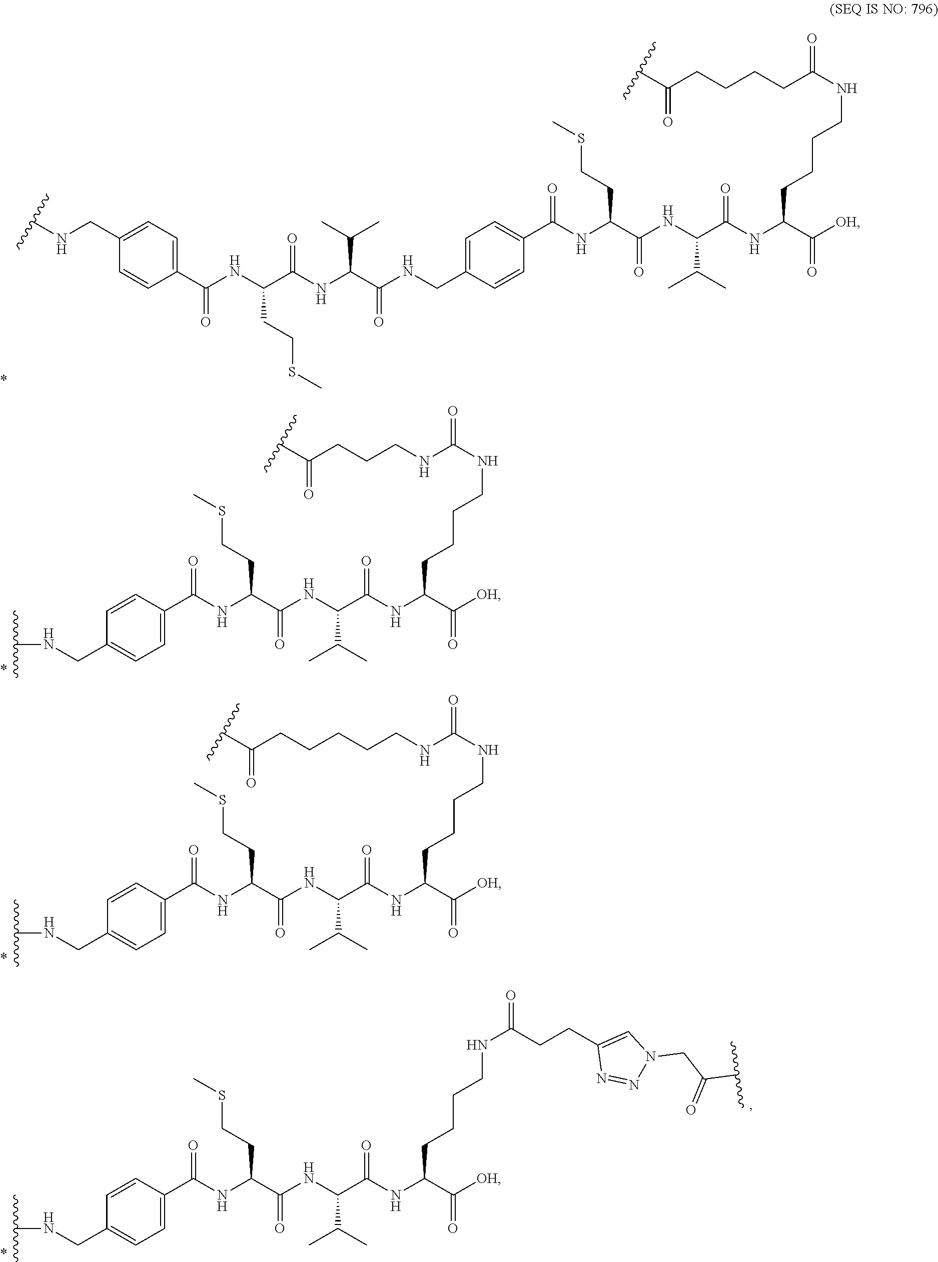 [SMILES: *NCc1ccc(C(=O)N[C@@H](CCSC)C(=O)N[C@H](C(=O)NCc2ccc(C(=O)N[C@@H](CCSC)C(=O)N[C@H](C(=O)N[C@@H](CCCCNC(=O)CCCCC(*)=O)C(=O)O)C(C)C)cc2)C(C)C)cc1 ; *NCc1ccc(C(=O)N[C@@H](CCSC)C(=O)N[C@H](C(=O)N[C@@H](CCCCNC(=O)NCCCC(*)=O)C(=O)O)C(C)C)cc1 ; *NCc1ccc(C(=O)N[C@@H](CCSC)C(=O)N[C@H](C(=O)N[C@@H](CCCCNC(=O)NCCCCCC(*)=O)C(=O)O)C(C)C)cc1 ; *NCc1ccc(C(=O)N[C@@H](CCSC)C(=O)N[C@H](C(=O)N[C@@H](CCCCNC(=O)CCc2cn(CC(*)=O)nn2)C(=O)O)C(C)C)cc1]

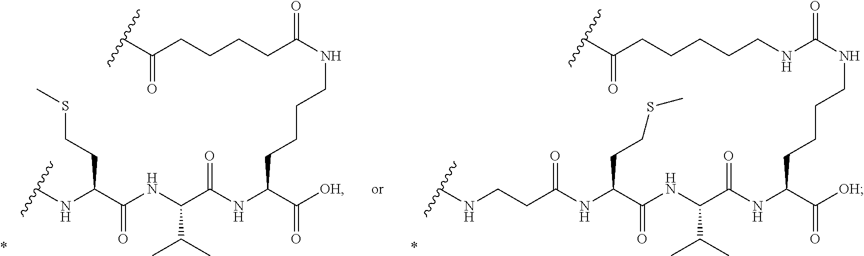 [SMILES: *N[C@@H](CCSC)C(=O)N[C@H](C(=O)N[C@@H](CCCCNC(=O)CCCCC(*)=O)C(=O)O)C(C)C ; *NCCC(=O)N[C@@H](CCSC)C(=O)N[C@H](C(=O)N[C@@H](CCCCNC(=O)NCCCCCC(*)=O)C(=O)O)C(C)C]

m is 1, 2, 3, 4, 5, or 6;
wherein * denotes the attachment point to R¹.

Embodiment 60. The compound of any one of embodiments 1-50, or a pharmaceutically acceptable salt thereof, wherein -L- is: absent,

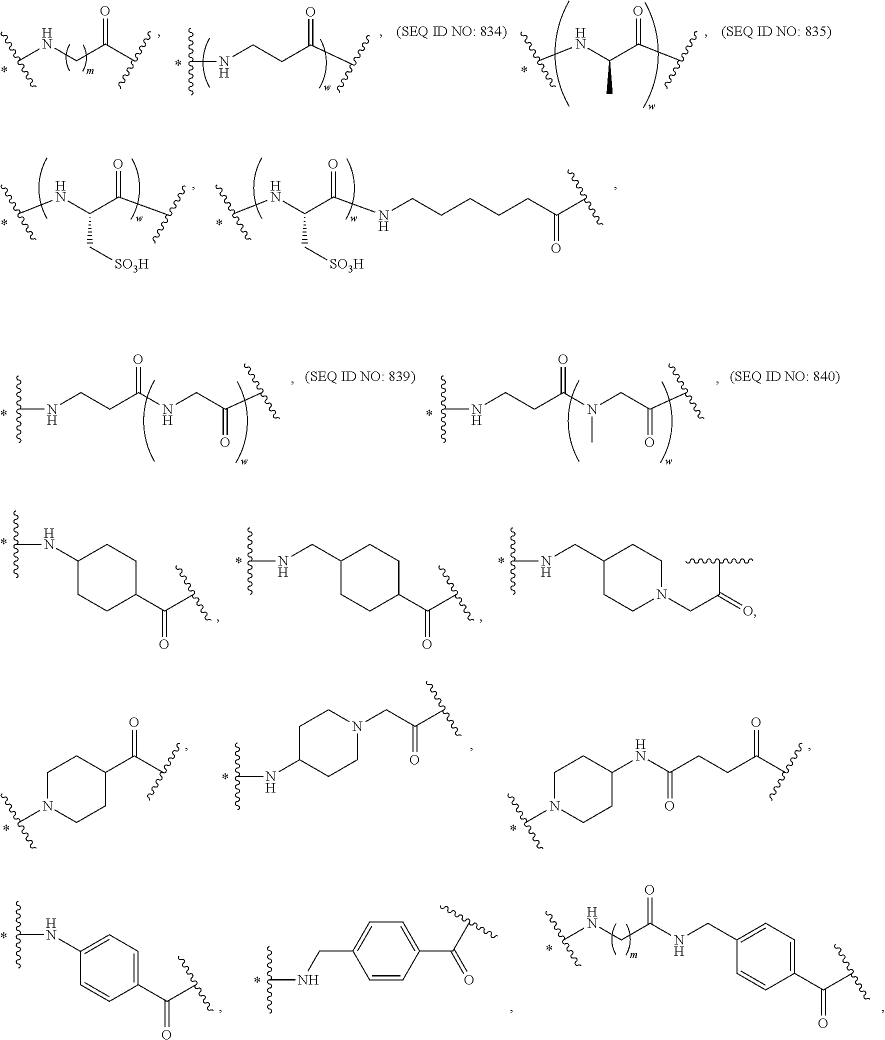

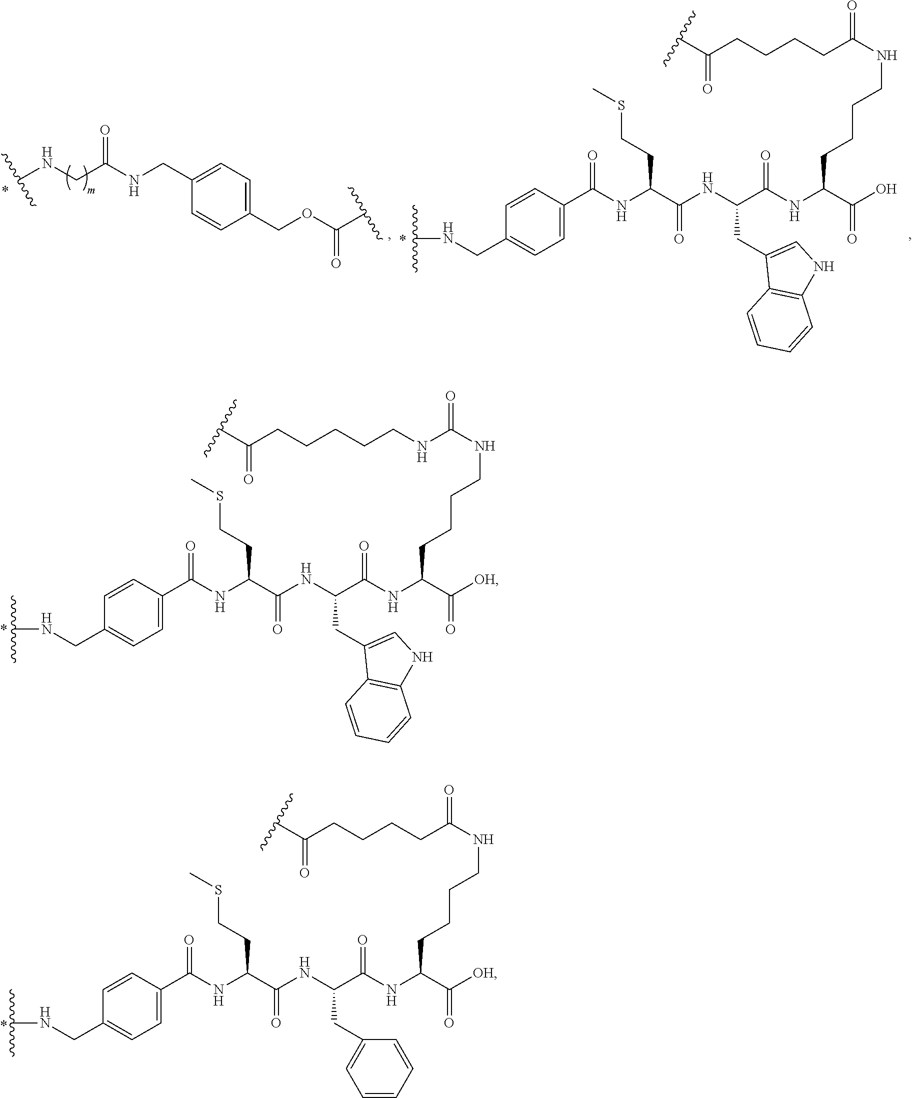

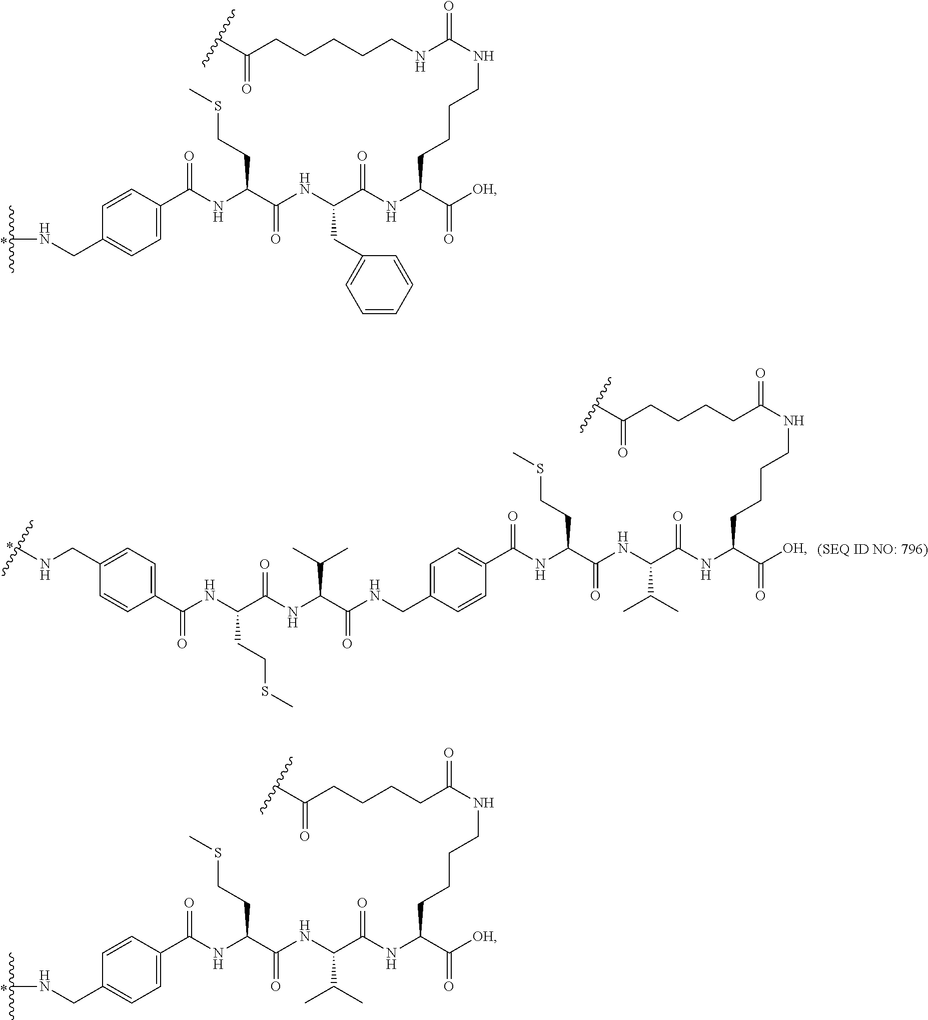 [SMILES: *NCc1ccc(C(=O)N[C@@H](CCSC)C(=O)N[C@@H](Cc2ccccc2)C(=O)N[C@@H](CCCCNC(=O)NCCCCCC(*)=O)C(=O)O)cc1 ; *NCc1ccc(C(=O)N[C@@H](CCSC)C(=O)N[C@H](C(=O)NCc2ccc(C(=O)N[C@@H](CCSC)C(=O)N[C@H](C(=O)N[C@@H](CCCCNC(=O)CCCCC(*)=O)C(=O)O)C(C)C)cc2)C(C)C)cc1 ; *NCc1ccc(C(=O)N[C@@H](CCSC)C(=O)N[C@H](C(=O)N[C@@H](CCCCNC(=O)CCCCC(*)=O)C(=O)O)C(C)C)cc1]

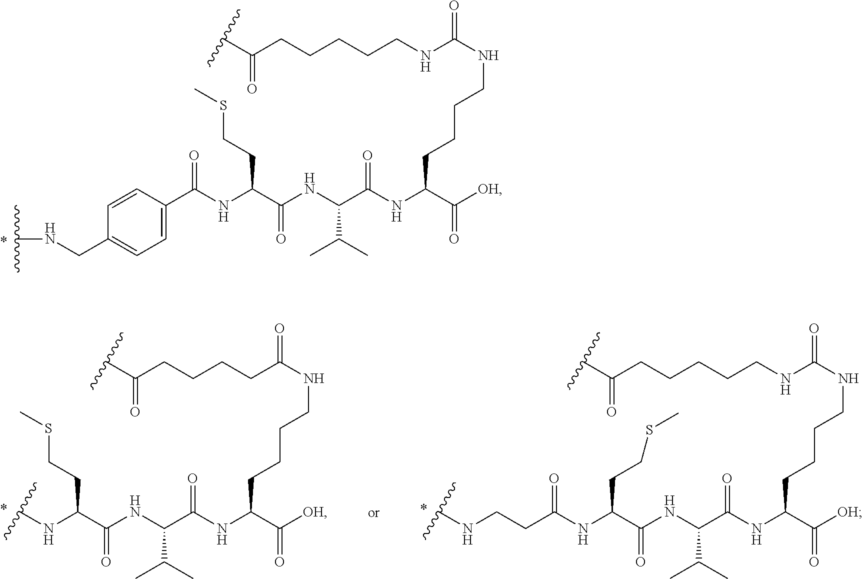 [SMILES: *NCc1ccc(C(=O)N[C@@H](CCSC)C(=O)N[C@H](C(=O)N[C@@H](CCCCNC(=O)NCCCCCC(*)=O)C(=O)O)C(C)C)cc1 ; *N[C@@H](CCSC)C(=O)N[C@H](C(=O)N[C@@H](CCCCNC(=O)CCCCC(*)=O)C(=O)O)C(C)C ; *NCCC(=O)N[C@@H](CCSC)C(=O)N[C@H](C(=O)N[C@@H](CCCCNC(=O)NCCCCCC(*)=O)C(=O)O)C(C)C]

m is 1, 2, 3, 4, 5, or 6;
wherein * denotes the attachment point to R^{a}.

Embodiment 61. The compound of any one of embodiments 58-60, or a pharmaceutically acceptable salt thereof, wherein:
m is 5.

Embodiment 62. The compound of any one of embodiments 1-50, or a pharmaceutically acceptable salt thereof, wherein -L- is:

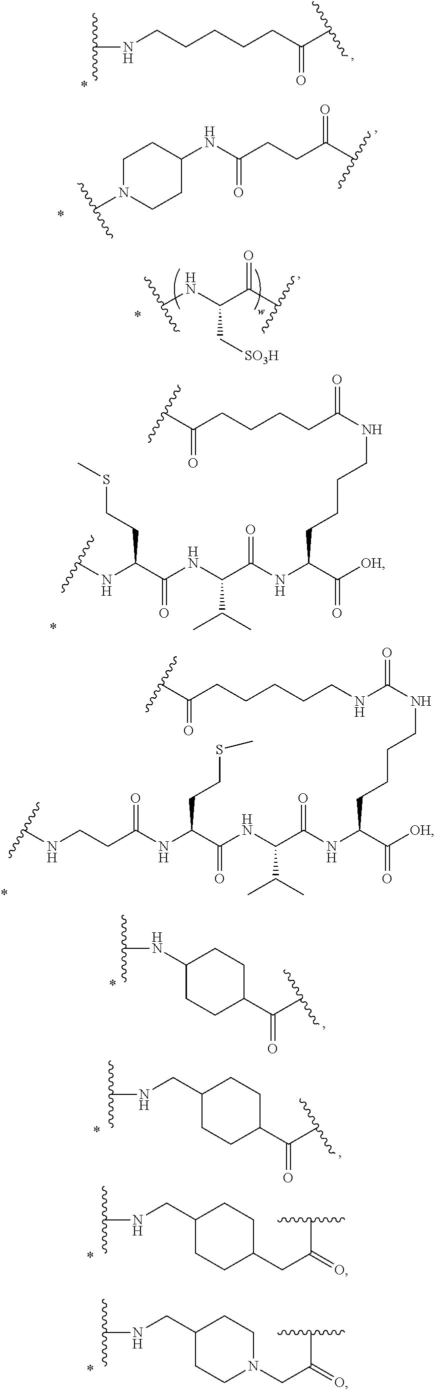

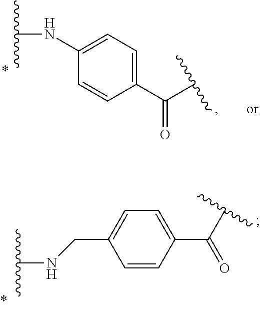 [SMILES: *Nc1ccc(C(*)=O)cc1 ; *NCc1ccc(C(*)=O)cc1]

wherein * denotes the attachment point to R^{a}.

Embodiment 63. The compound of any one of embodiments 1-50, or a pharmaceutically acceptable salt thereof, wherein: -L- is:

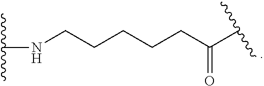 [SMILES: *NCCCCCC(*)=O]

Embodiment 64. The compound of any one of embodiments 1-50, or a pharmaceutically acceptable salt thereof, wherein R^{a}-L- is

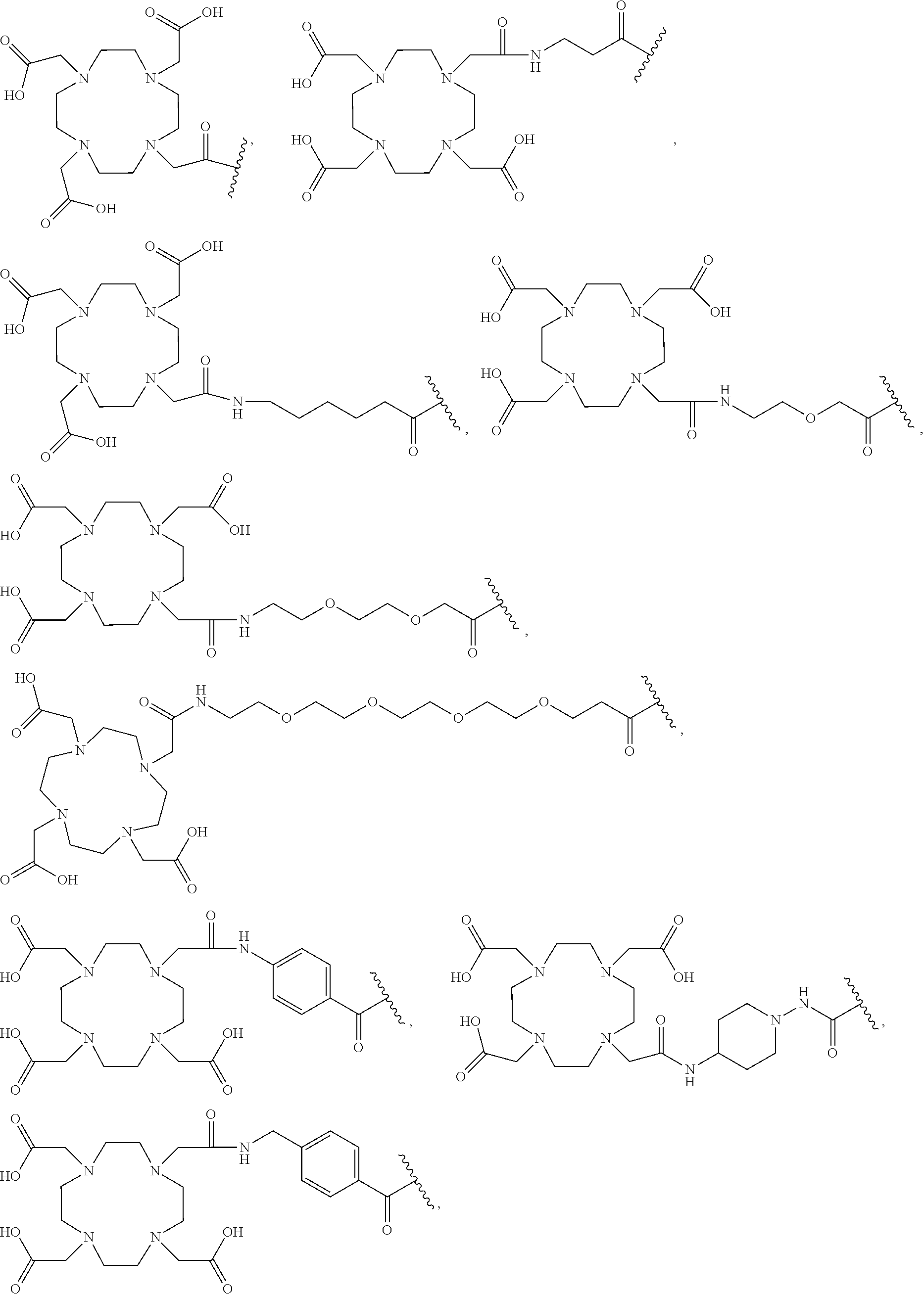

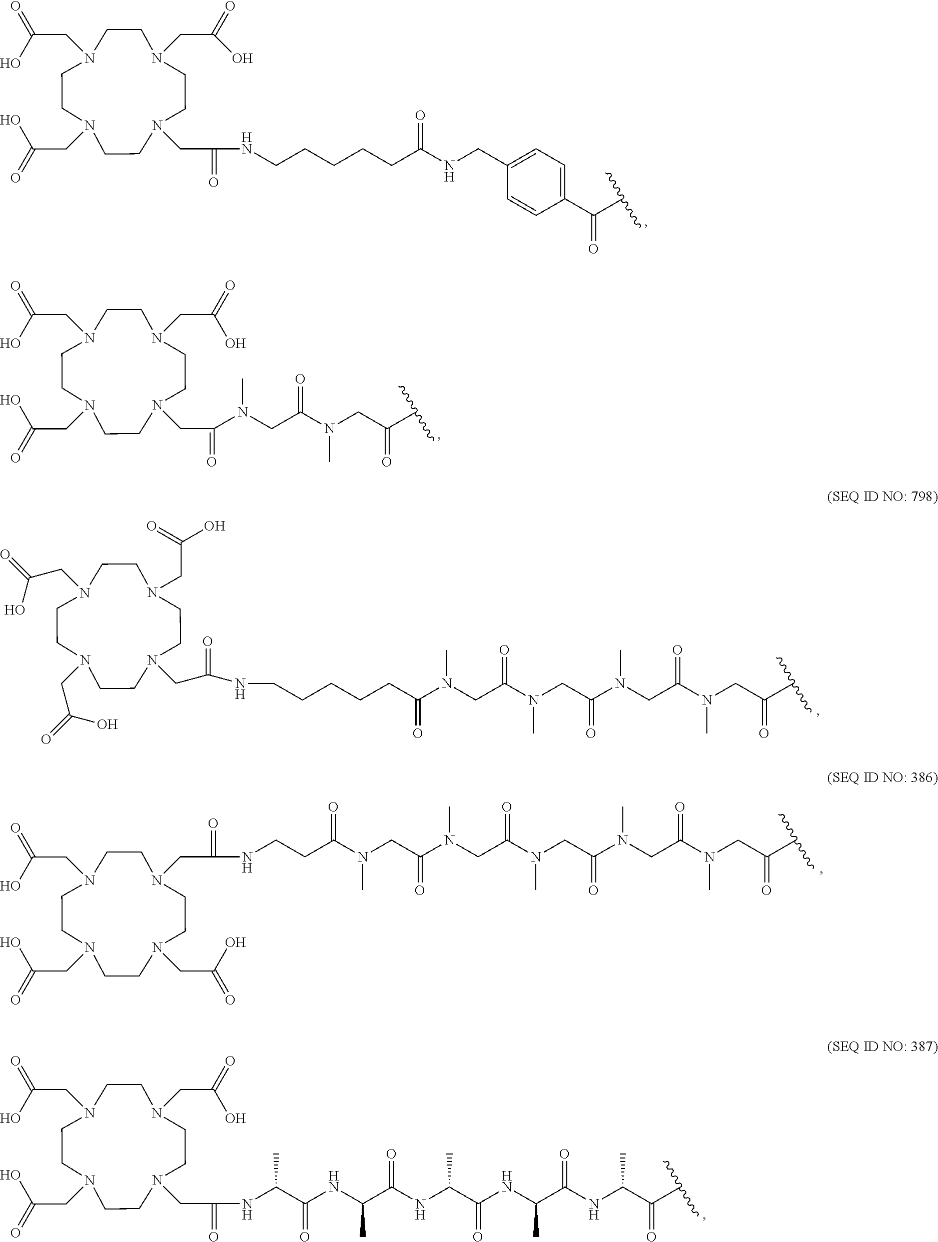 [SMILES: *C(=O)c1ccc(CNC(=O)CCCCCNC(=O)CN2CCN(CC(=O)O)CCN(CC(=O)O)CCN(CC(=O)O)CC2)cc1 ; *C(=O)CN(C)C(=O)CN(C)C(=O)CN1CCN(CC(=O)O)CCN(CC(=O)O)CCN(CC(=O)O)CC1 ; *C(=O)CN(C)C(=O)CN(C)C(=O)CN(C)C(=O)CN(C)C(=O)CCCCCNC(=O)CN1CCN(CC(=O)O)CCN(CC(=O)O)CCN(CC(=O)O)CC1 ; *C(=O)CN(C)C(=O)CN(C)C(=O)CN(C)C(=O)CN(C)C(=O)CN(C)C(=O)CCNC(=O)CN1CCN(CC(=O)O)CCN(CC(=O)O)CCN(CC(=O)O)CC1 ; *C(=O)[C@@H](C)NC(=O)[C@@H](C)NC(=O)[C@@H](C)NC(=O)[C@@H](C)NC(=O)[C@@H](C)NC(=O)CN1CCN(CC(=O)O)CCN(CC(=O)O)CCN(CC(=O)O)CC1]

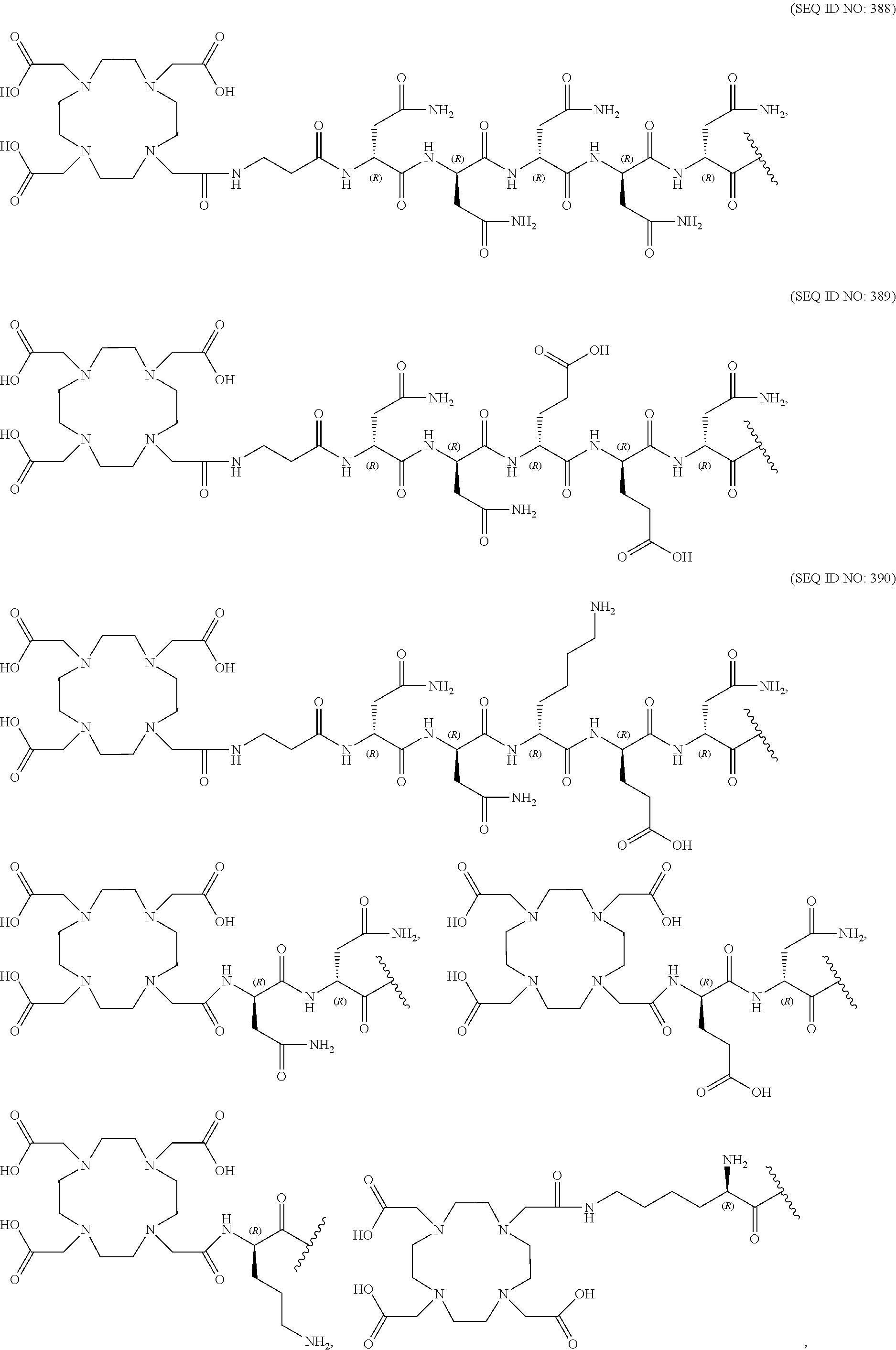

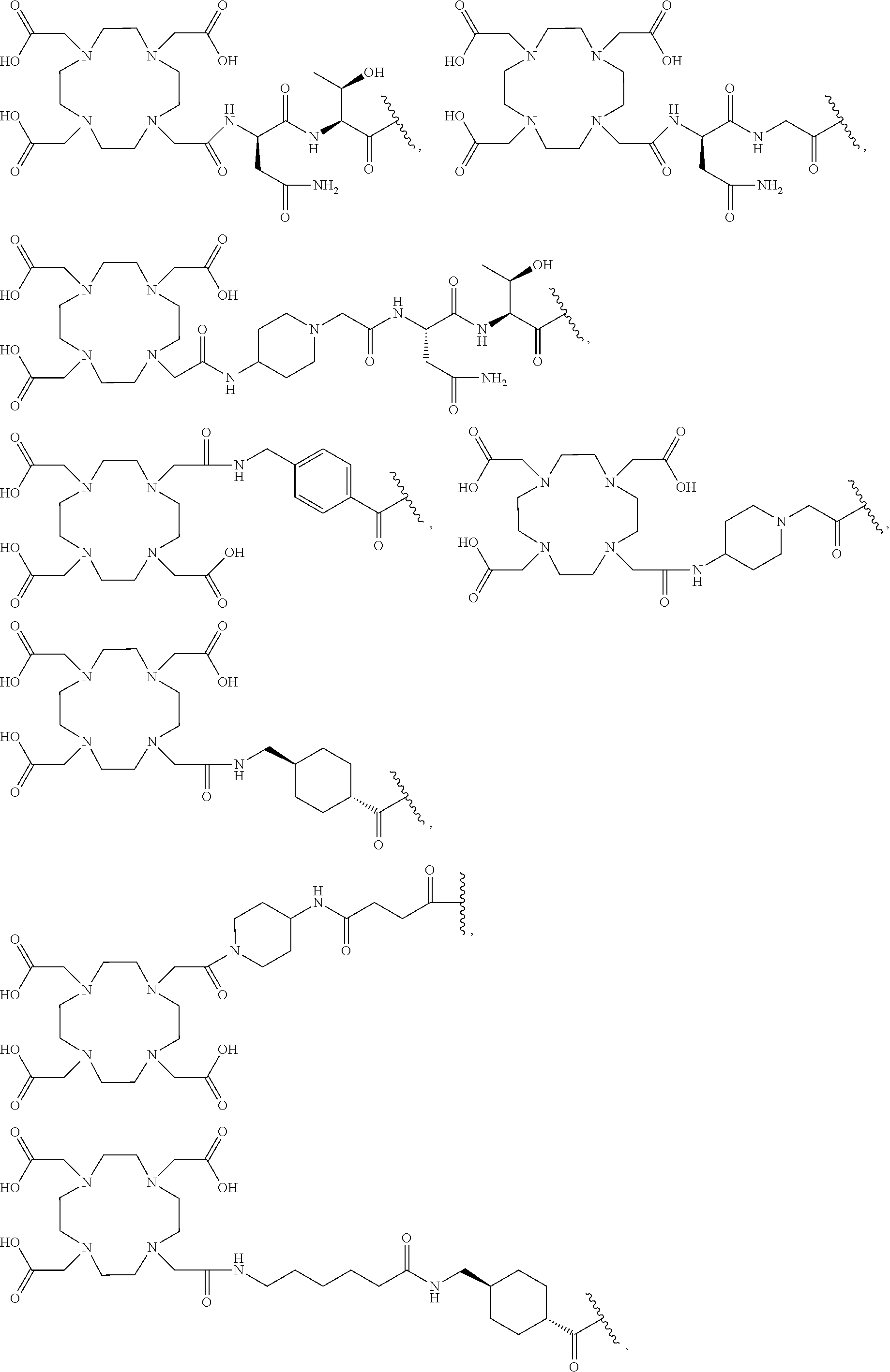

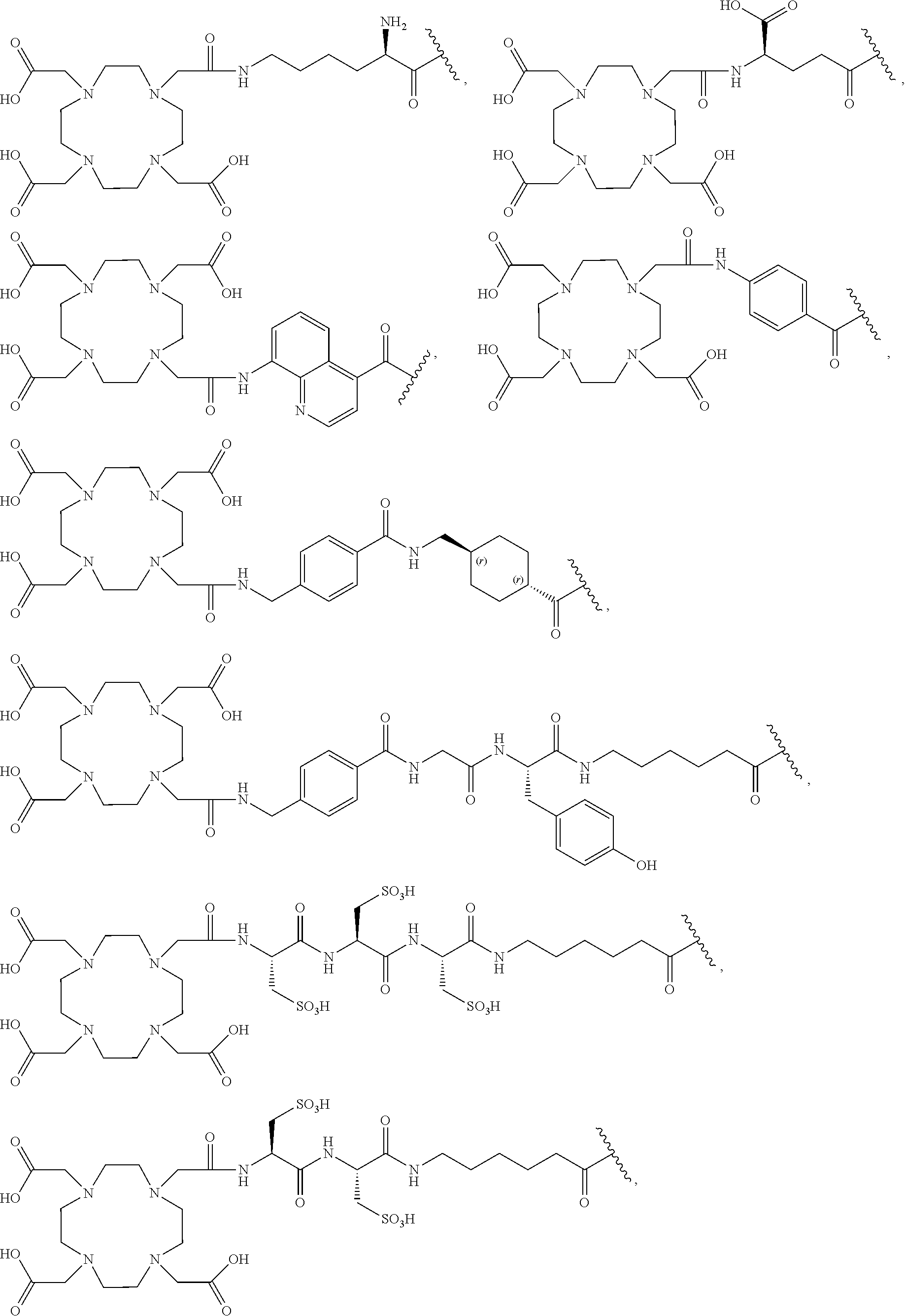

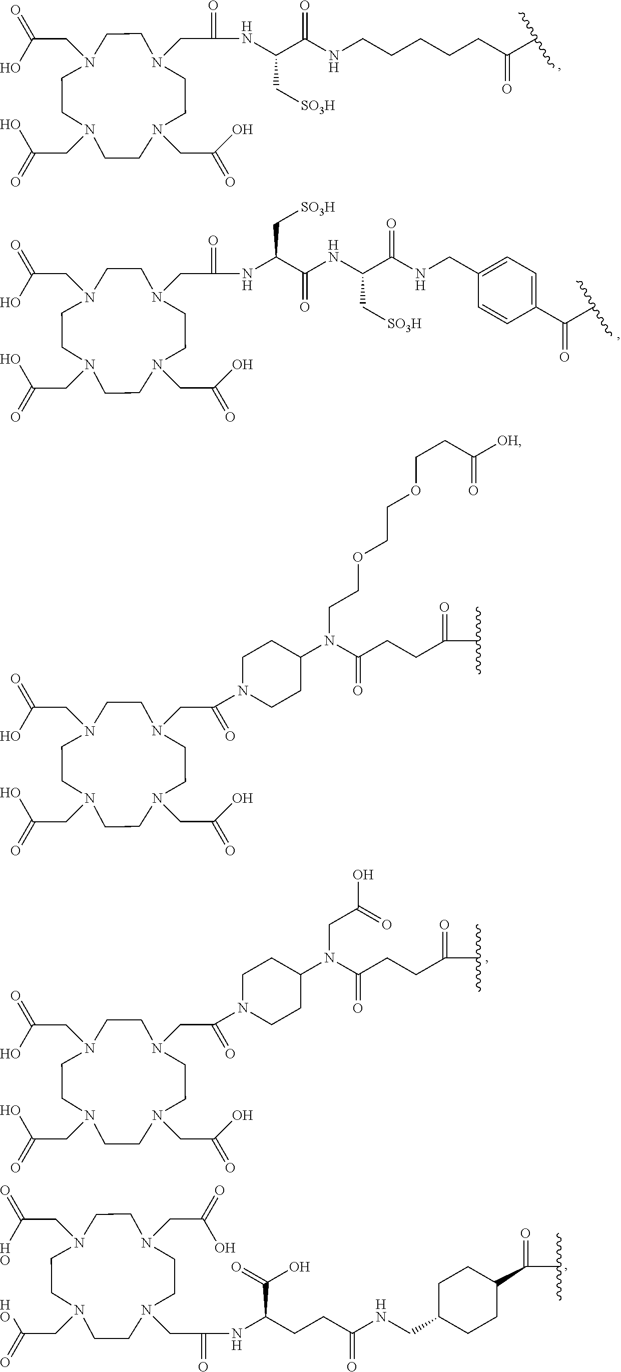

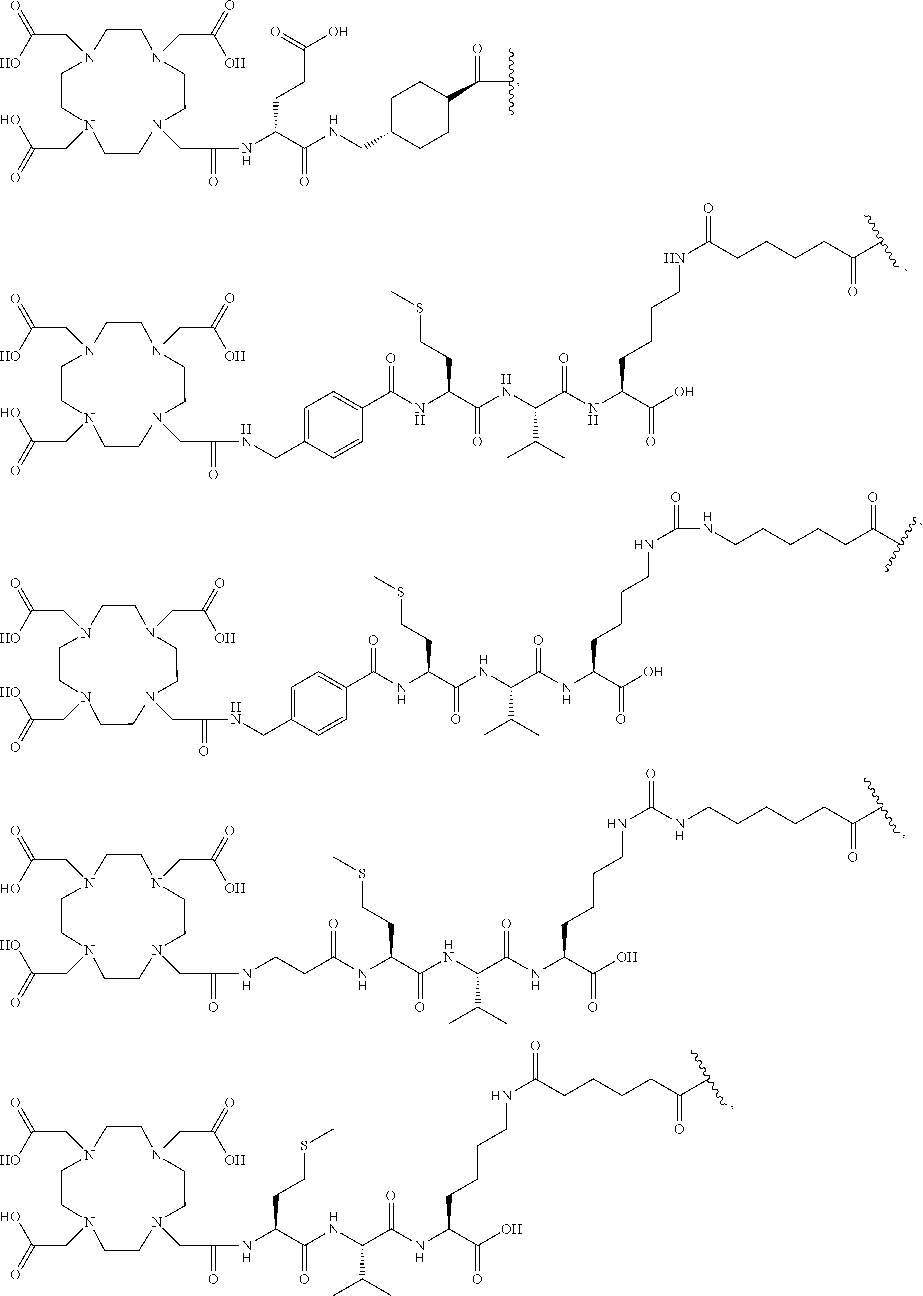

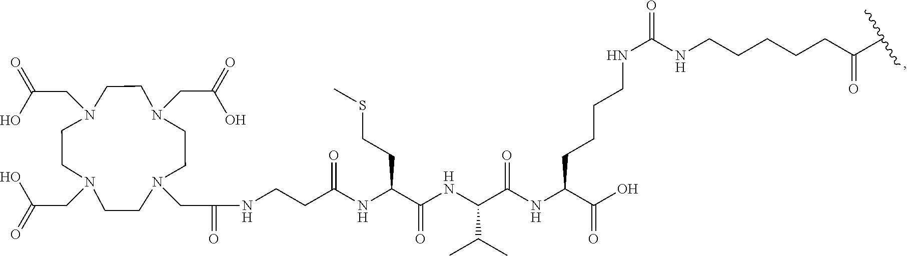 [SMILES: *C(=O)CCCCCNC(=O)NCCCC[C@H](NC(=O)[C@@H](NC(=O)[C@H](CCSC)NC(=O)CCNC(=O)CN1CCN(CC(=O)O)CCN(CC(=O)O)CCN(CC(=O)O)CC1)C(C)C)C(=O)O]

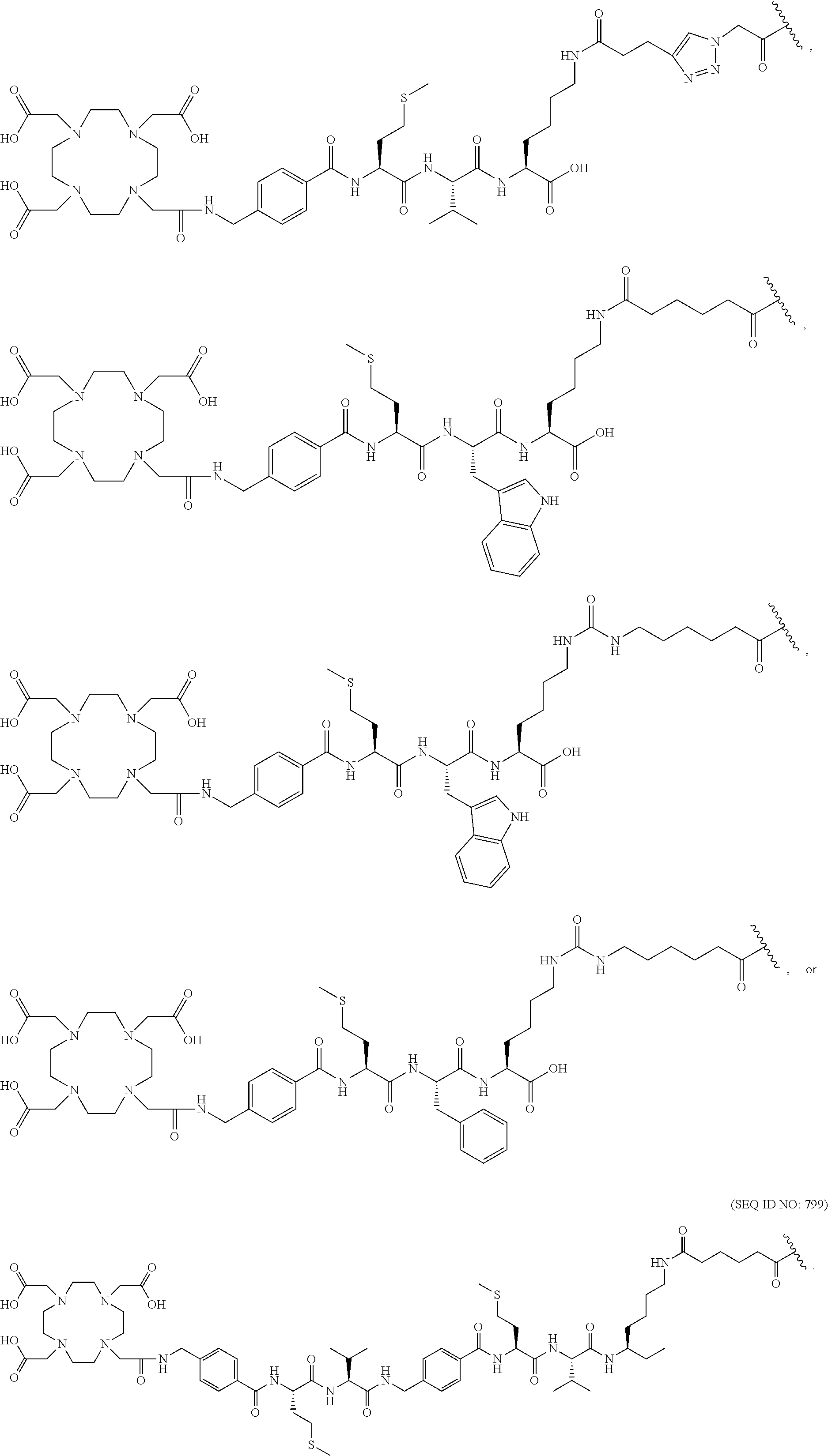

Embodiment 65. The compound of embodiment 1, or a pharmaceutically acceptable salt thereof, wherein the compound of Formula (I) has the chemical structure corresponding to one of the following SEQ ID numbers, or a pharmaceutically acceptable salt thereof:

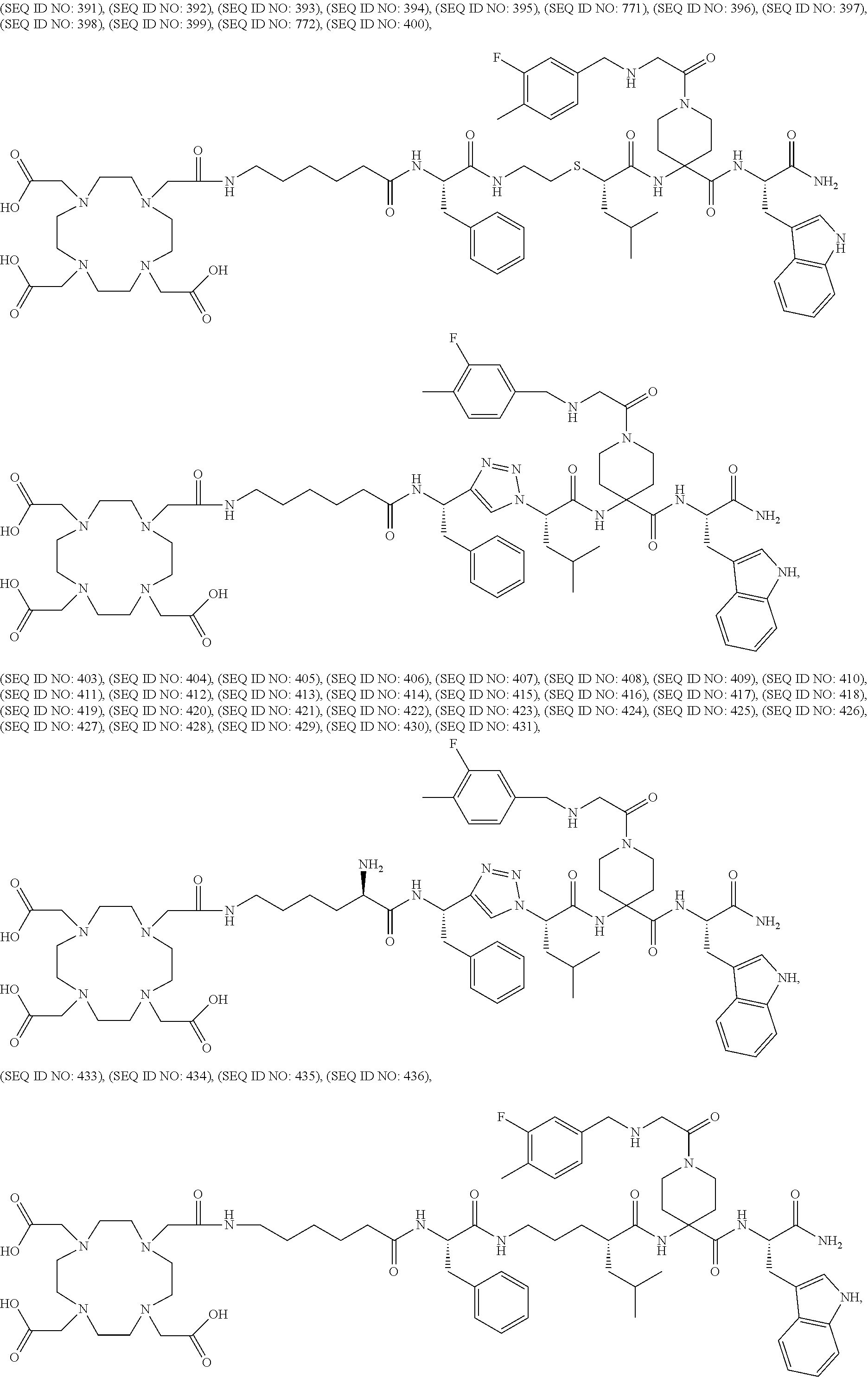

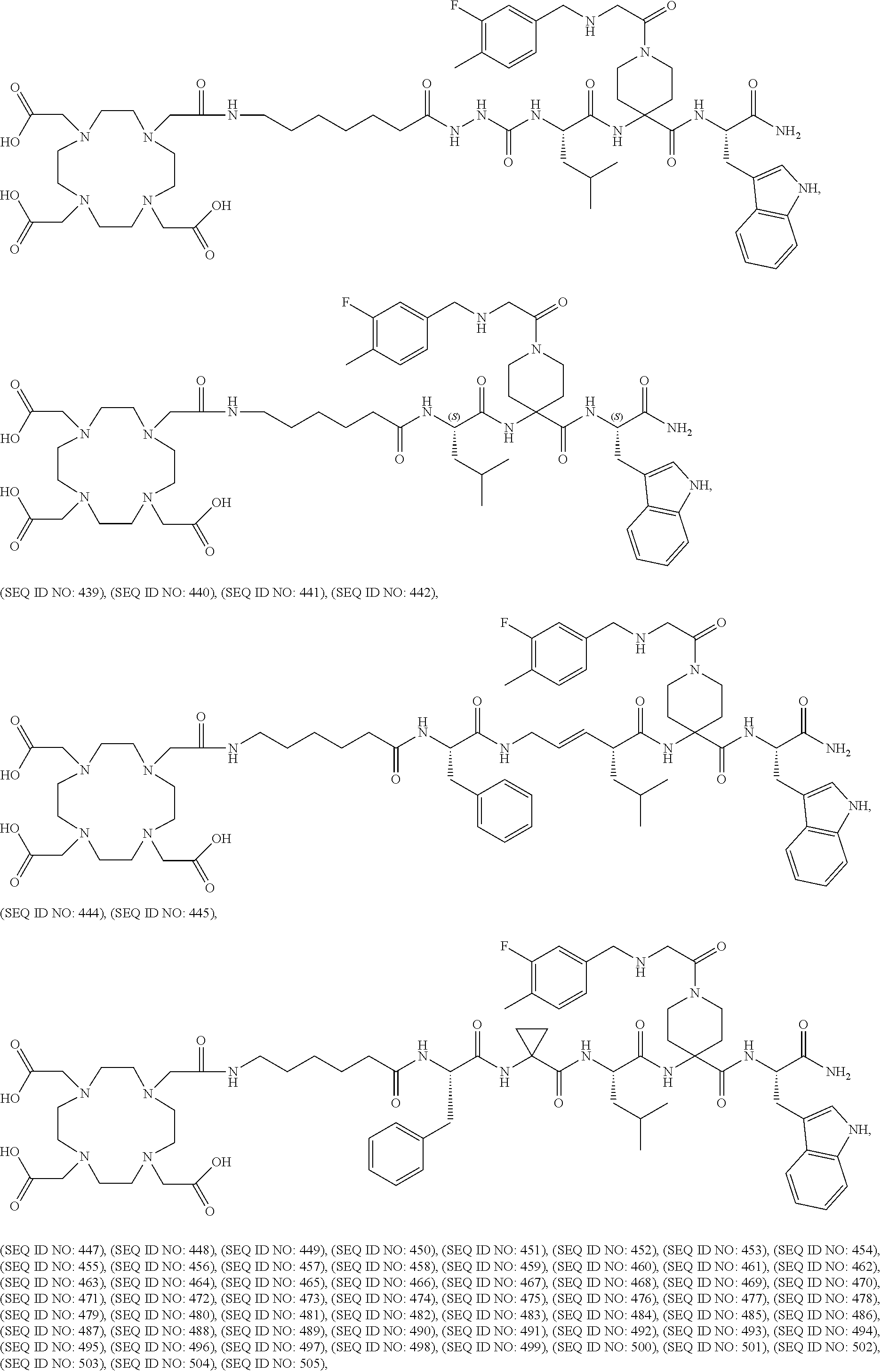

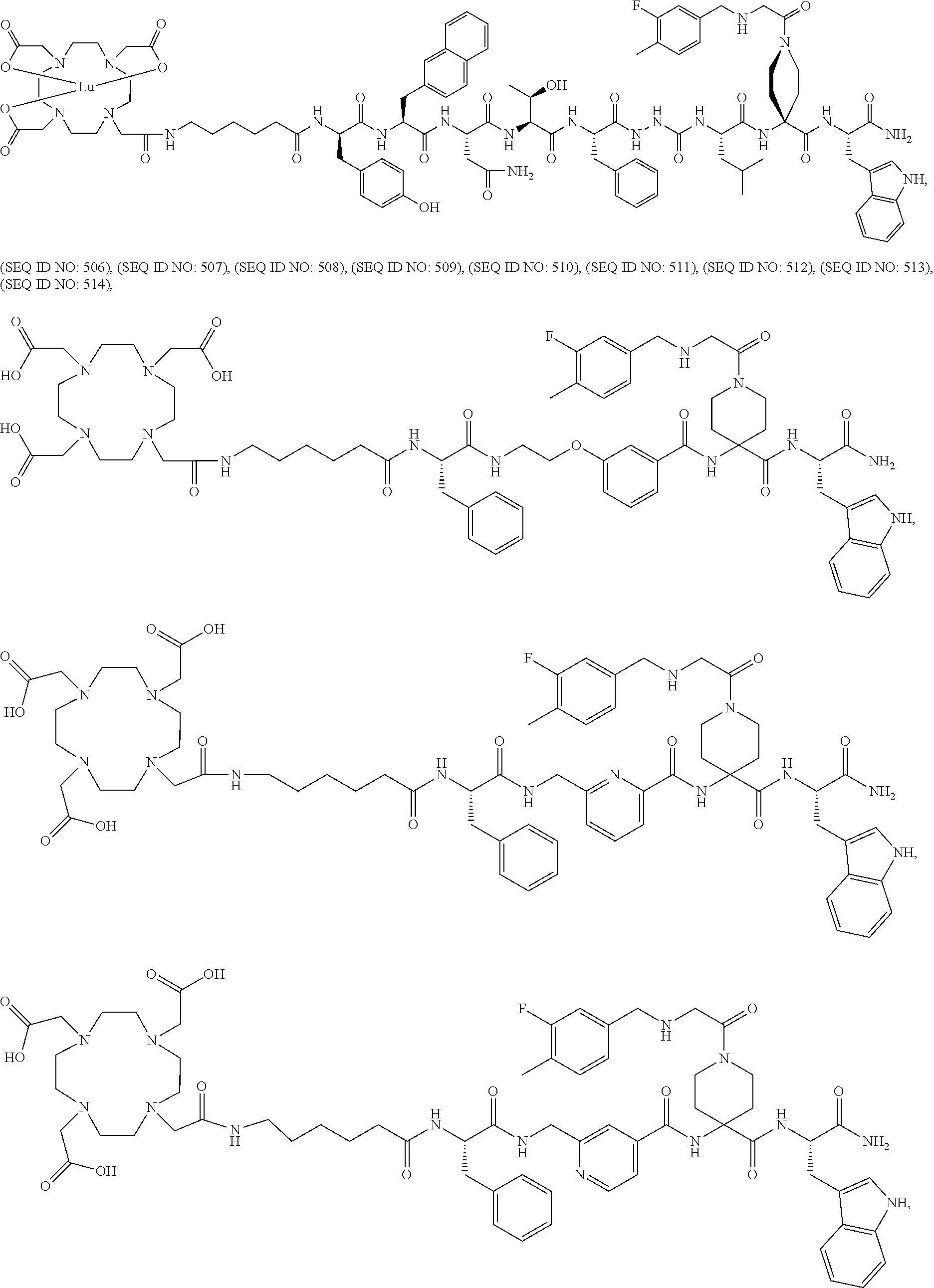

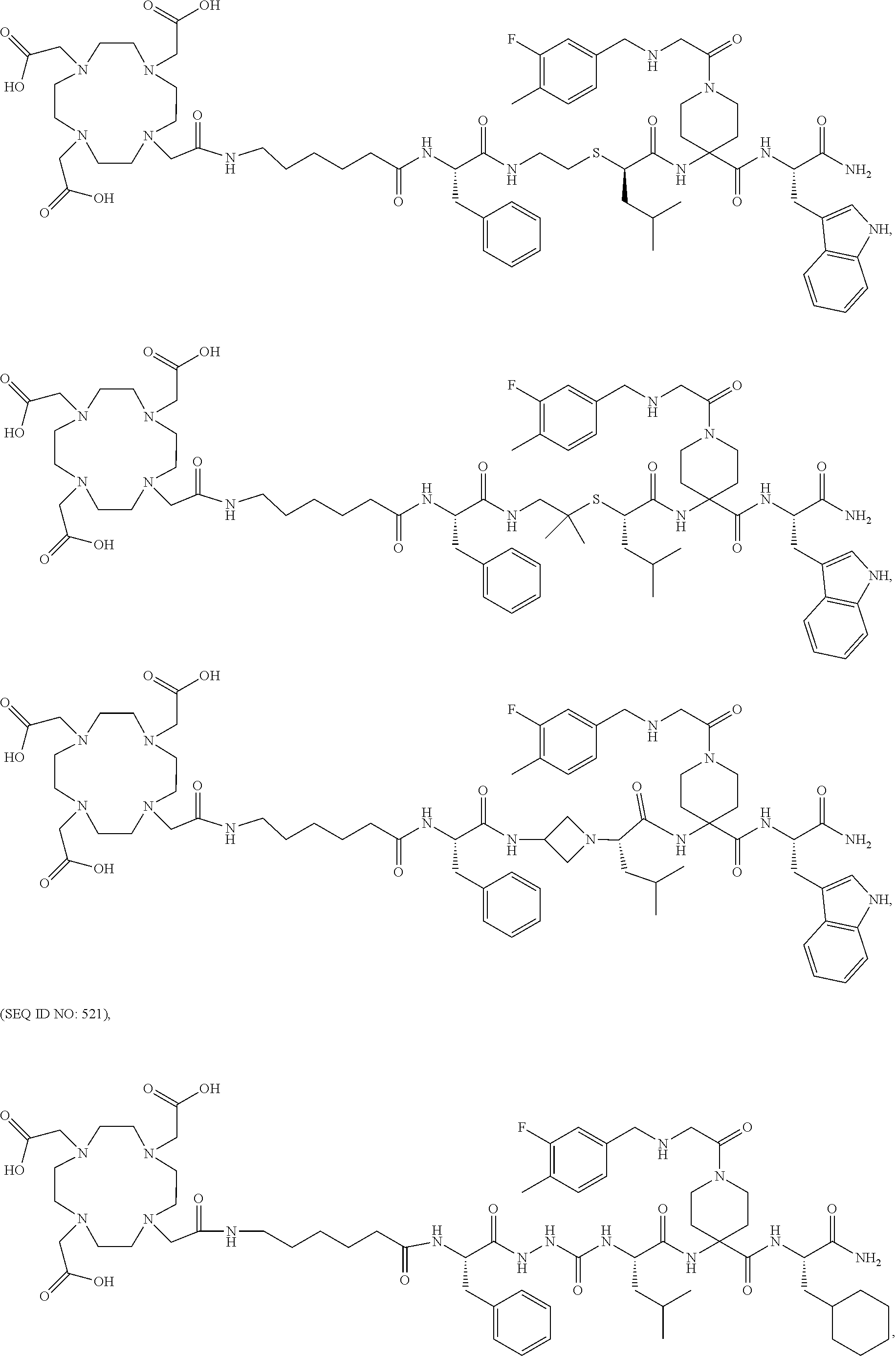

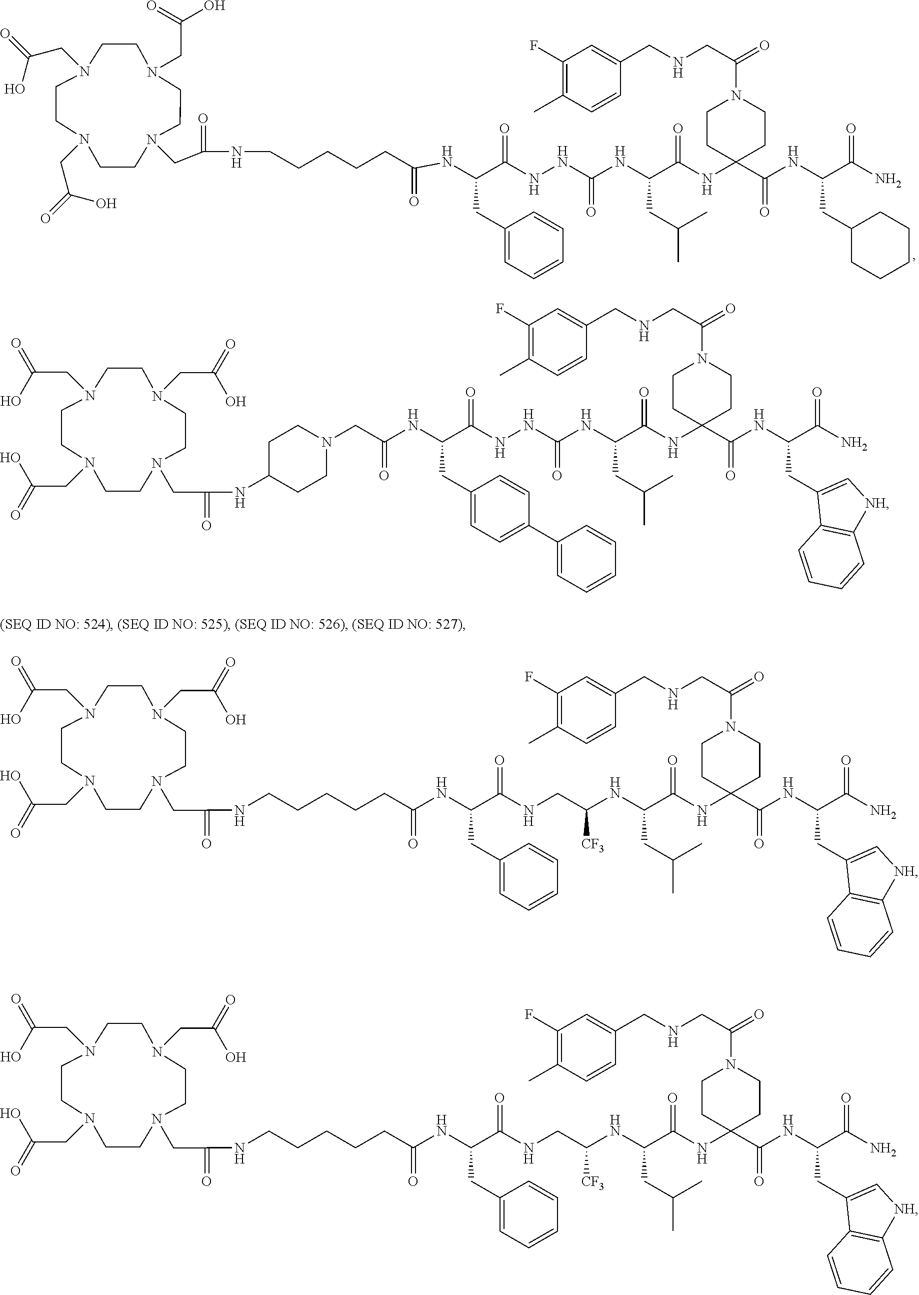

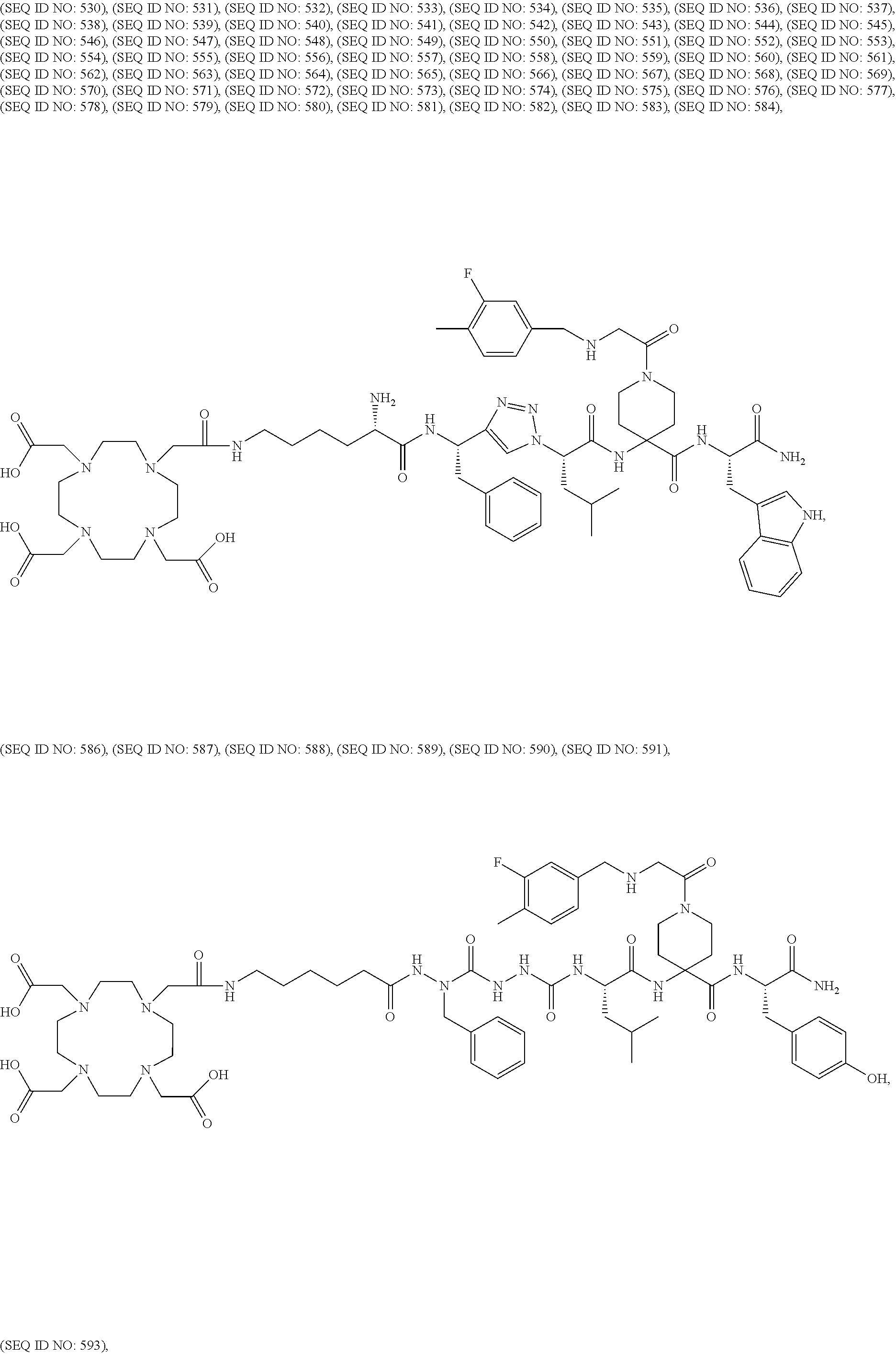 [SMILES: Cc1ccc(CNCC(=O)N2CCC(NC(=O)[C@H](CC(C)C)n3cc([C@H](Cc4ccccc4)NC(=O)[C@@H](N)CCCCNC(=O)CN4CCN(CC(=O)O)CCN(CC(=O)O)CCN(CC(=O)O)CC4)nn3)(C(=O)N[C@@H](Cc3c[nH]c4ccccc34)C(N)=O)CC2)cc1F ; Cc1ccc(CNCC(=O)N2CCC(NC(=O)[C@H](CC(C)C)NC(=O)NNC(=O)N(Cc3ccccc3)NC(=O)CCCCCNC(=O)CN3CCN(CC(=O)O)CCN(CC(=O)O)CCN(CC(=O)O)CC3)(C(=O)N[C@@H](Cc3ccc(O)cc3)C(N)=O)CC2)cc1F]

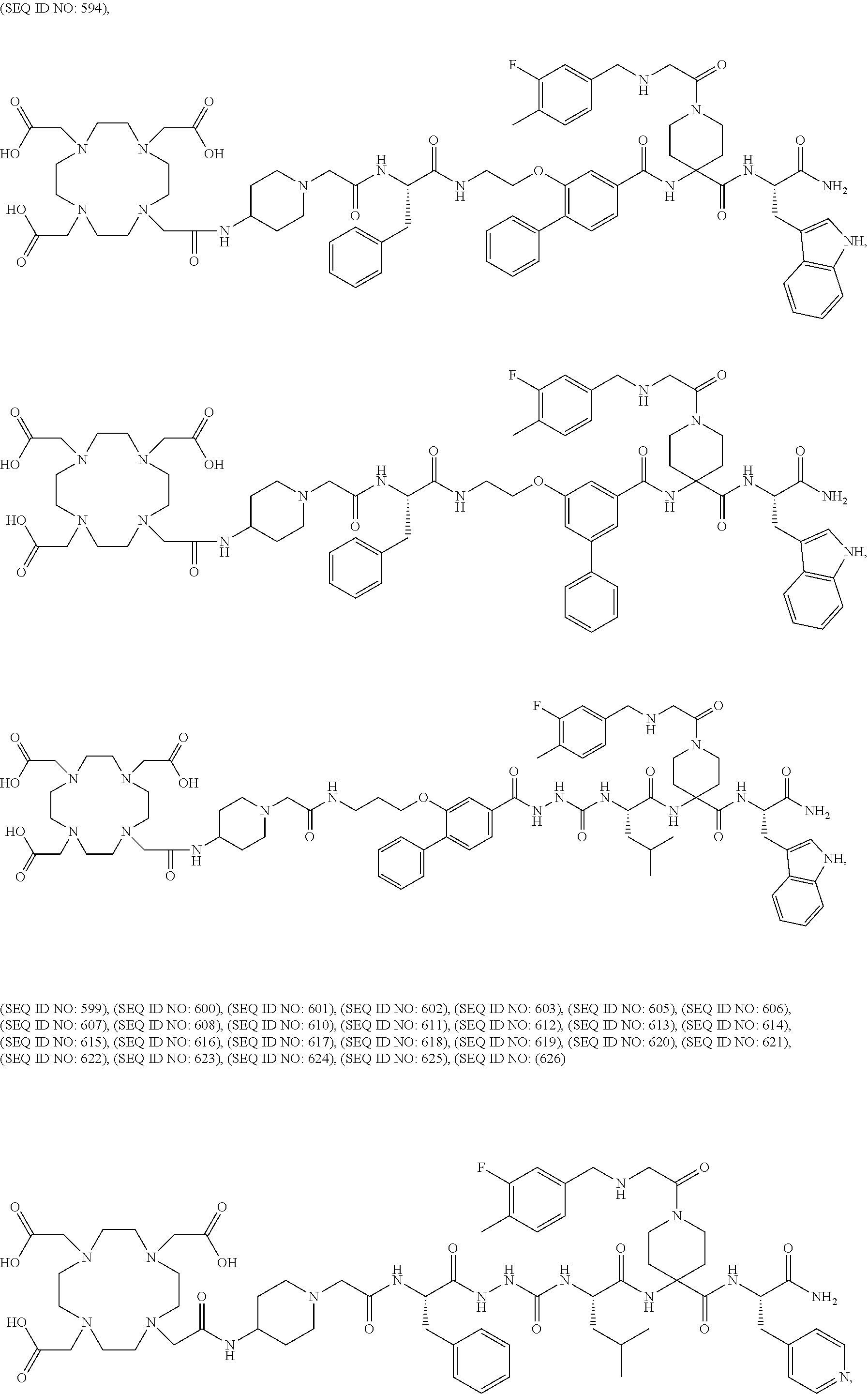

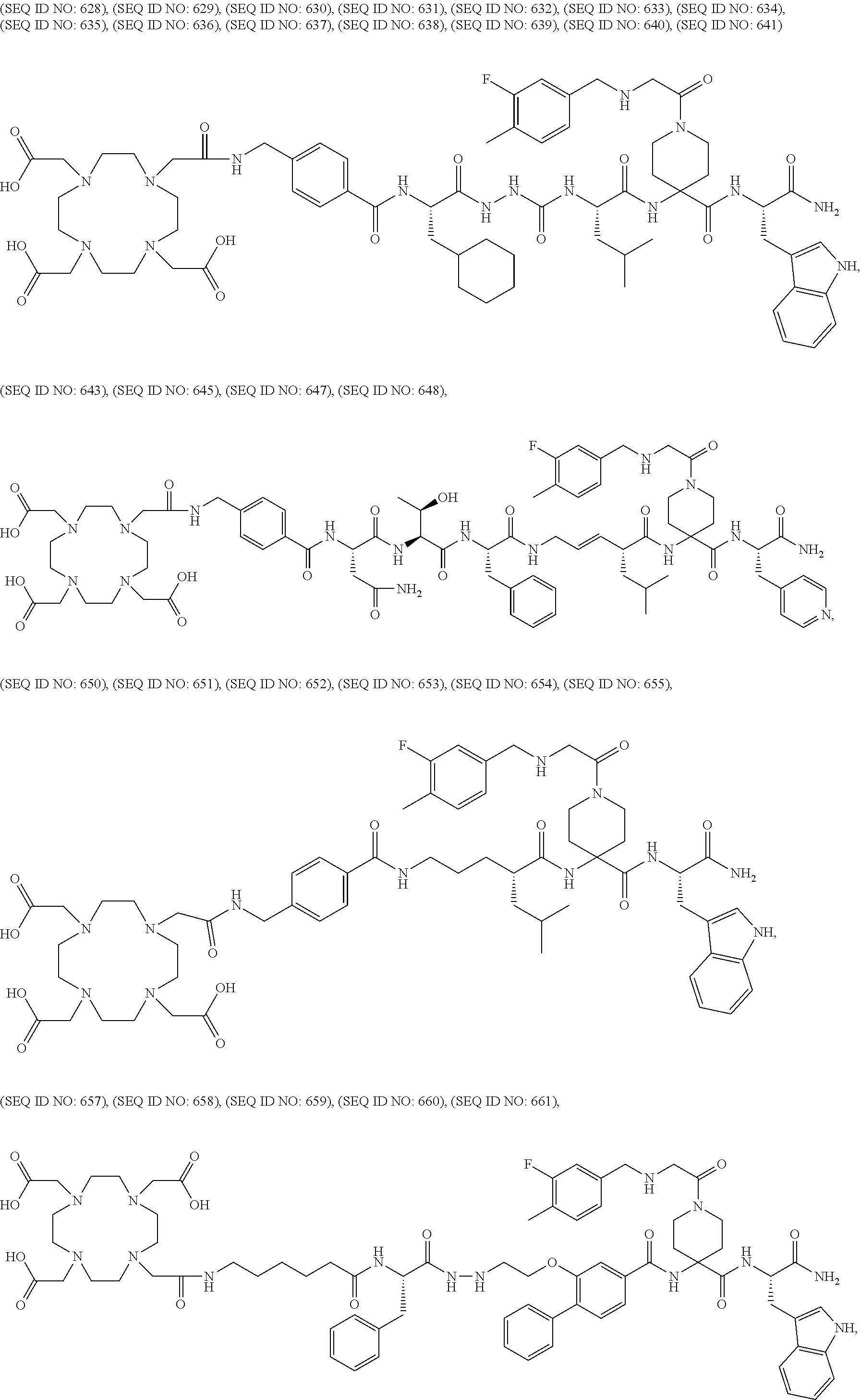

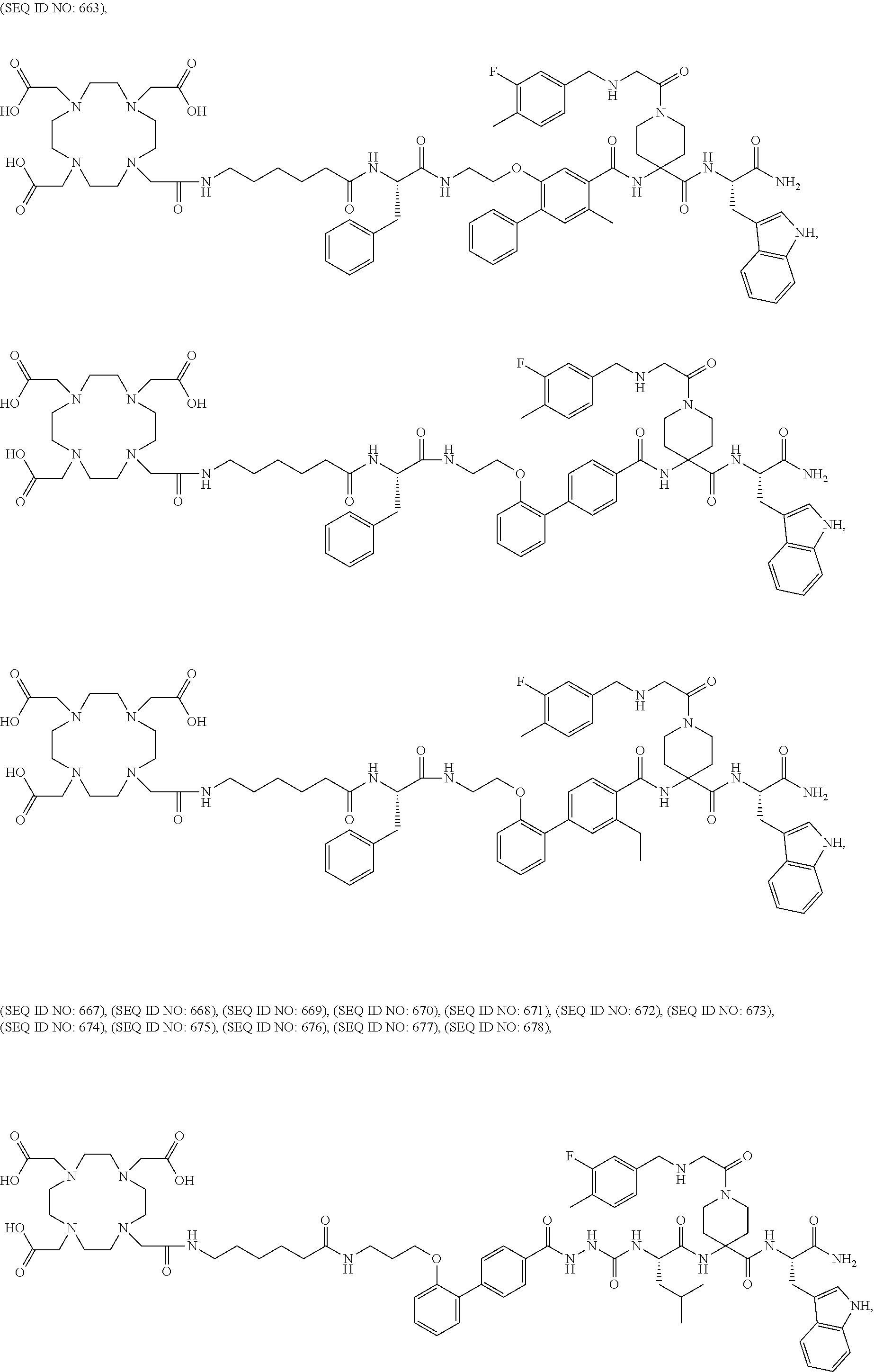

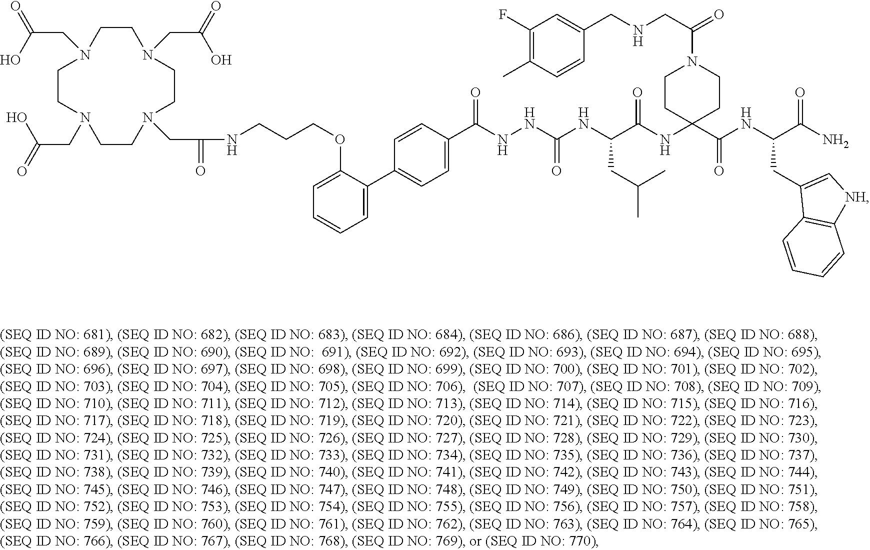 [SMILES: Cc1ccc(CNCC(=O)N2CCC(NC(=O)[C@H](CC(C)C)NC(=O)NNC(=O)c3ccc(-c4ccccc4OCCCNC(=O)CN4CCN(CC(=O)O)CCN(CC(=O)O)CCN(CC(=O)O)CC4)cc3)(C(=O)N[C@@H](Cc3c[nH]c4ccccc34)C(N)=O)CC2)cc1F]

or a radionuclide complex thereof.

Embodiment 66. The compound of any one of embodiments 1-65, or a pharmaceutically acceptable salt thereof, wherein: the radionuclide of the radionuclide complex is a lanthanide or an actinide.

Embodiment 67. The compound of any one of embodiments 1-65, or a pharmaceutically acceptable salt thereof, wherein: the radionuclide of the radionuclide complex is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium.

Embodiment 68. The compound of any one of embodiments 1-65, or a pharmaceutically acceptable salt thereof, wherein: the radionuclide of the radionuclide complex is a diagnostic or therapeutic radionuclide.

Embodiment 69. The compound of any one of embodiments 1-65, or a pharmaceutically acceptable salt thereof, wherein: the radionuclide of the radionuclide complex is an Auger electron-emitting radionuclide, α-emitting radionuclide, β-emitting radionuclide, or 7-emitting radionuclide.

Embodiment 70. The compound of any one of embodiments 1-65, or a pharmaceutically acceptable salt thereof, wherein the radionuclide of the radionuclide complex is:
an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{195m}Pt); or
an α-emitting radionuclide that is 225-actinium (²²⁵Ac), 213-bismuth (²¹³Bi), 223-Radium (²²³Ra), or 212-lead (²¹²Pb); or
a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (39Zr), or 52-manganese (⁵²Mn) or a 7-emitting radionuclide that is 60-cobalt (⁶⁰Co), 103-palldium (¹⁰³Pd), 137-cesium (¹³⁷Cs), 169-ytterbium (¹⁶⁹Yb), 192-iridium (¹⁹²Ir), or 226-radium (²²⁶Ra).

Embodiment 71. The compound of any one of embodiments 1-65, or a pharmaceutically acceptable salt thereof, wherein: the radionuclide of the radionuclide complex is 111-indium (¹¹¹In), 67-gallium (⁶⁷Ga), 68-gallium (⁶⁸Ga), 69-gallium (69Ga), 71-gallium (⁷¹Ca), 225-actinium (²⁵⁵Ac), 175-lutetium (¹⁷⁵Lu), 177-lutetium (¹⁷⁷Lu), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), or 67-copper (⁶⁷Cu).

Embodiment 72. The compound of any one of embodiments 1-65, or a pharmaceutically acceptable salt thereof, wherein: the radionuclide of the radionuclide complex is 64-copper(⁴Cu), 67-copper (⁶⁷Cu), 90-yttrium (⁹⁰Y), 111-indium (¹¹¹In), 67-gallium (⁶⁷Ga 68-gallium (⁶⁸Ga), 225-actinium (²²Ac), or 177-lutetium (¹⁷⁷Lu) or 212-lead (²¹²Pb).

Embodiment 73. A pharmaceutical composition comprising a compound of any one of embodiments 1-72, or a pharmaceutically acceptable salt thereof, and at least one pharmaceutically acceptable excipient.

Embodiment 74. The pharmaceutical composition of embodiment 73, wherein the pharmaceutical composition is formulated for administration to a mammal by intravenous administration.

Embodiment 75. A method for the treatment of cancer comprising administering to a mammal with cancer an effective amount of a compound of any one of embodiments 1-72, or a pharmaceutically acceptable salt thereof.

Embodiment 76. The method of embodiment 75, wherein the cancer comprises tumors and the tumors overexpress Kisspeptin receptor (KISS1R).

Embodiment 77. The method of embodiment 75 or embodiment 76, wherein the cancer is glioma, thyroid cancer, lung cancer, colorectal cancer, stomach cancer, liver cancer, pancreatic cancer, renal cancer, prostate cancer, testis cancer, breast cancer, cervical cancer, endometrial cancer, ovarian cancer, or melanoma.

Embodiment 78. The method of embodiment 75 or embodiment 76, wherein the cancer is breast cancer, renal cancer, or lung cancer.

Embodiment 79. A method of killing tumors in a mammal that overexpress Kisspeptin receptor (KISS1R) comprising administering to the mammal a compound of any one of embodiments 1-72, or a pharmaceutically acceptable salt thereof, wherein the compound of any one of embodiments 1-72, or a pharmaceutically acceptable salt thereof, comprises a therapeutic radionuclide.

Embodiment 80. The method of embodiment 79, wherein the mammal has been diagnosed with glioma, thyroid cancer, lung cancer, colorectal cancer, stomach cancer, liver cancer, pancreatic cancer, renal cancer, prostate cancer, testis cancer, breast cancer, cervical cancer, endometrial cancer, ovarian cancer, or melanoma.

Embodiment 81. The method of embodiment 79, wherein the mammal has been diagnosed with breast cancer, renal cancer, or lung cancer.

Embodiment 82. A method for identifying tumors expressing Kisspeptin receptor (KISS1R) in a mammal comprising administering to the mammal a compound of any one of embodiments 1-72, or a pharmaceutically acceptable salt thereof; and performing positron emission tomography (PET) analysis, single-photon emission computerized tomography (SPECT), or magnetic resonance imaging (MRI) wherein the compound of any one of embodiments 1-72, or a pharmaceutically acceptable salt thereof, comprises a diagnostic radionuclide.

Embodiment 83. A method for the in vivo imaging of tissues or organs in a mammal with tumors expressing the Kisspeptin receptor (KISS1R) comprising administering to the mammal a compound of any one of embodiments 1-72, or a pharmaceutically acceptable salt thereof; and performing positron emission tomography (PET) analysis, single-photon emission computerized tomography (SPECT), or magnetic resonance imaging (MRI); wherein the compound of any one of embodiments 1-72, or a pharmaceutically acceptable salt thereof, comprises a diagnostic radionuclide.

Embodiment 84. A compound of Formula (I I), or a pharmaceutically acceptable salt thereof:

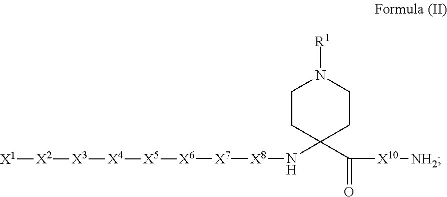

wherein:
R¹ is H,

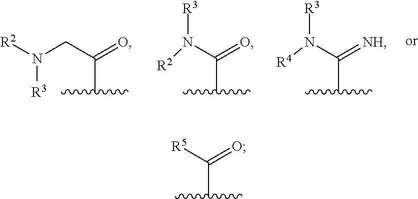

R² is C₁-C₈ alkyl, substituted or unsubstituted heteroalkyl, —(CHR⁶)ₙ-heterocycloalkyl, —(CHR⁶)ₙ-aryl, —(CHR⁶)ₙ-heteroaryl, —C(═O)—(CHR⁶)ₙ-aryl, or —C(═O)NH—(CHR⁶)ₙ-aryl; Wherein C₁-C₆ alkyl is optionally substituted with R⁷, and wherein the heterocycloalkyl, aryl, or heteroaryl are each independently optionally substituted with R⁷, R⁸, R⁹, R¹⁰, and R¹¹;
R³ is H or C₁-C₄ alkyl;
R⁴ is H, C₁-C₄ alkyl, or R²;
R⁵ is substituted or unsubstituted alkyl, substituted or unsubstituted heteroalkyl, or —(CHR⁶)ₙ-aryl; wherein aryl is optionally substituted with R⁷, R⁸, R⁹, R¹⁰, and R¹¹;
each R⁶ is independently H, F, —CH₃, —NH₂, or —OH;
R⁷, R⁸, ⁹, R¹⁰, and R¹¹ are each independently selected from H, F, Cl, Br, L—OH, —O—C₁-C₄ alkyl, —NH₂, —NHC₁-C₄ alkyl, —N(C₁-C₄ alkyl)₂, —CN, —CO₂H, —CO₂C₁-C₄ alkyl, —C₁-C₆ alkyl, —C₁-C₆ fluroroalkyl or —C₃-C₆ cycloalkyl;
   n is 0, 1, 2, 3, 4, 5, or 6;
X¹ is absent, tyrosine (Tyr), glycine (Gly), sarcosine (Sar), alanine (Ala), aspartic acid (Asp), lysine (Lys), phenylalanine (Phe), 3-(3-pyridyl)alanine (3-Pal) threonine (Thr), methionine (Met), 4-iodophenylalanine (Phe(4-4)), N6-(4-(p-tolyl)butanoyl)-lysine, N6-(4-(4-iodophenyl)butanoyl)-lysine, or γ-glutamic acid (γ-Glu));
X² is absent, asparagine (Asn), glutamine (Gln), aspartic acid (Asp), glutamic acid (Glu), serine (Ser), histidine (His), alanine (Ala), sarcosine (Sar), tyrosine (Tyr), proline (Pro), hydroxyproline (Hyp), azetidine-2-carboxylic acid (Aze), 2,3,4,5-tetrahydroisoquinoline-3-carboxylic acid (Tic), phenylalanine (Phe), 3-(2-Pyridyl)alanine (2-Pal), 3-(3-Pyridyl)alanine (3-Pal), or 3-(4-pyridyl)alanine (4-Pal);
X³ is absent, tryptophan (Trp), serine (Ser), leucine (Leu), isoleucine (Ile), phenylalanine (Phe), 4-iodophenylalanine (Phe(4-I)), 3-(2-pyridyl)alanine (2-Pal), 3-(3-pyridyl)alanine (3-Pal), 3-(4-pyridyl)alanine (4-Pal), 2-amino-3-(naphthalen-2-yl)propanoic acid (H-2-NAL-OH), lysine (Lys), asparagine (Asn), glutamine (Gln), aspartic acid (Asp), glutamic acid (Glu), arginine (Arg), methyl arginine (Arg(Me)), norarginine (AGBA), methyl norarginine (AGBA(Me)), homoarginine (HArg), methyl homoarginine (HArg(Me)), citrulline (Cit), methyl citrulline (Cit(Me)), canavanine, methyl-canavanine, glycine (Gly), alanine (Ala), sarcosine (Sar), tyrosine (Tyr), cyclohexylalanine (Cha), 3-(1-naphthyl)alanine(α-Nal), 3-(2-naphthyl)alanine (β-Nal), or threonine (Thr), proline (Pro), hydroxyproline (Hyp), or tetrahydroisoquinoline-3-carboxylic acid (Tic); O-phospho-serine (SOP), 2-amino-4-(2H-tetrazol-5-yl)butanoic acid, β-glutamnic acid, 8-aminoquinoline-3-carboxylic acid, biphenylalanine (Bip), 4-benzoylphenylalanine (Bpa), or 3-(9-anthryl)-alanine (H-Ala(9-Anth)-OH or AAP);
X⁴ is absent, asparagine (Asn), glutamine (Gln), aspartic acid (Asp), glutamic acid (Glu), tryptophan (Trp), glycine ((Gly), tyrosine (Tyr), alanine (Ala), or sarcosine (Sar);
X⁵ is absent, serine (Ser), threonine (Thr), lysine (Lys), asparagine (Asn), glutamine (Gln), aspartic acid (Asp), glutamic acid (Glu), glycine (Gly), alanine (Ala), sarcosine (San) or arginine (Arg);
X⁶ is absent, phenylalanine (Phe), alpha-methylphenylalanine (α-Me-Phe), N-methylphenylalanine (N-Me-Phe), 2-fluorophenylalanine (2-F-Phe), 3-fluorophenylalanine (3-F-Phe), 4-fluorophenylalanine (4-F-Phe), 4-iodophenylalanine (Phe(4-I)), 2-amino-2-indancarboxylic acid (Aic), biphenylalanine (Bip), (β-(2-thienyl)-Ala), tryptophan (Trp), 2-aminotetralin-2-carboxylic acid (Atc); 3-(2-thienyl)-alanine, 3-(4-pyridyl)alanine (4-Pal), cyclohexylalanine (Cha), or tyrosine (Tyr);
X⁷ is absent, glycine (Gly), aza-glycine (aza-Gly), alanine (Ala), N-methylglycine (Sar), or 1-aminocyclopropane-1-carboxylic acid (ACC);
X⁸ is leucine (Leu) norvaline (Nva), valine (Val), isoleucine (Ile), homoalanine (HAla), tryptophan (Trp), phenylalanine (Phe), or phenylglycine (Phg);
or —X⁷—X⁸— is

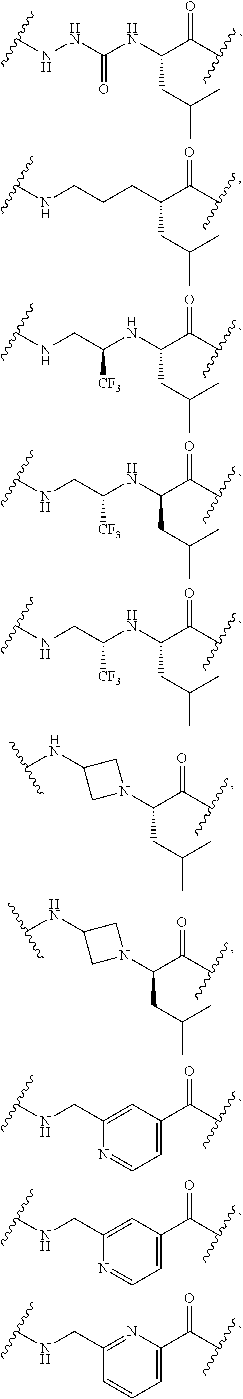

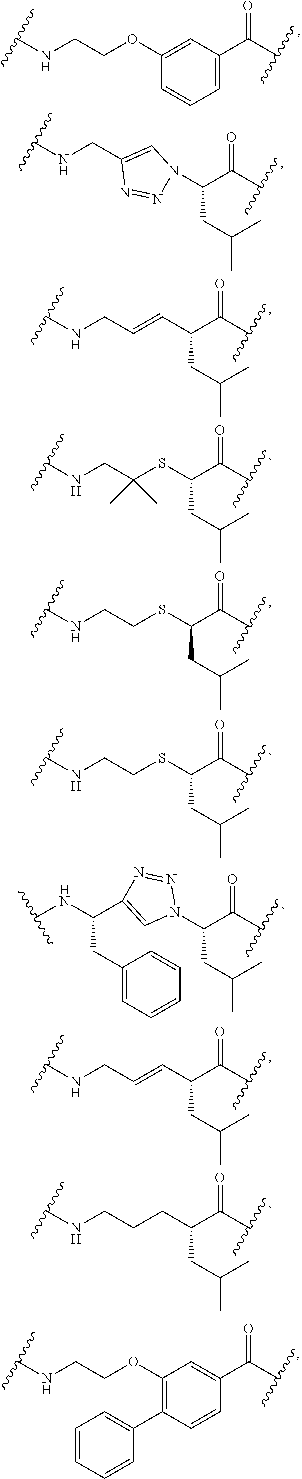

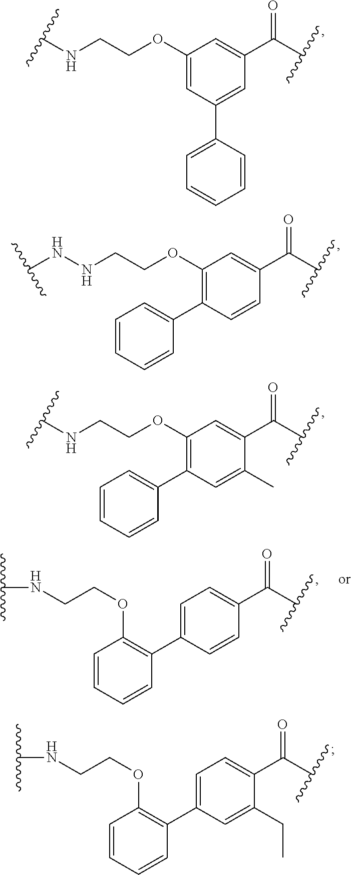 [SMILES: *NCCOc1cc(C(*)=O)cc(-c2ccccc2)c1 ; *NNCCOc1cc(C(*)=O)ccc1-c1ccccc1 ; *NCCOc1cc(C(*)=O)c(C)cc1-c1ccccc1 ; *NCCOc1ccccc1-c1ccc(C(*)=O)cc1 ; *NCCOc1ccccc1-c1ccc(C(*)=O)c(CC)c1]

or —X⁶—X⁷—X⁸— is

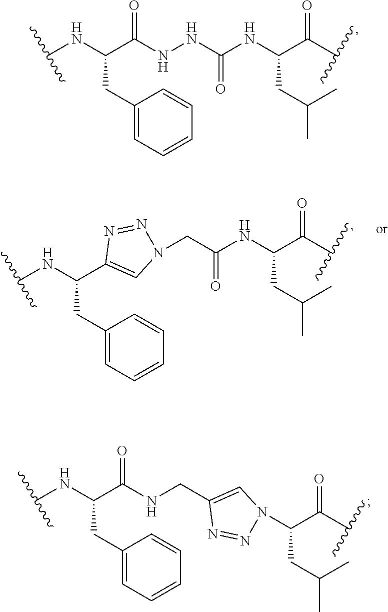 [SMILES: *N[C@@H](Cc1ccccc1)C(=O)NNC(=O)N[C@@H](CC(C)C)C(*)=O ; *N[C@@H](Cc1ccccc1)c1cn(CC(=O)N[C@@H](CC(C)C)C(*)=O)nn1 ; *N[C@@H](Cc1ccccc1)C(=O)NCc1cn([C@@H](CC(C)C)C(*)=O)nn1]

X¹⁰ is tryptophan (Trp), 1-methyltryptophan (1MT), tyrosine (Tyr), phenylalanine (Phe), 4-cyano phenylalanine (Phe(4-CN)), 3-(4-pyridyl)alanine (4-Pal), leucine (Leu), phenylglycine (Phg), cyclohexylalanine (Cha), 3-(1-naphthyl)alanine (α-Nal), 3-(2-naphthyl)-alanine (β-Nal), histidine (His), or 3-nitro-tyrosine (Tyr(3-NO₂)); wherein the N-terminal amino acid or the compound of Formula (II) is optionally substituted with —C(═O)—C₁-C₂₀ alkyl, —C(═O)—(CH₂CH₂O)_{y}—CH₂CH₂—R¹⁵, —C₁-C₂₀ alkyl, N-hexadecanoyl-Glu, —C₄-C₂₀ polyethylene glycol, a saccharide, —R¹⁶, —C(═O)—(CH₂CH₂)ₓ—CH₃, —C(═O)—(CH₂CH₂O)ₓ—H, —C(═O)CH₂CH₂(COOH)—R¹⁵, —C(═O)—(CH₂)₂R¹⁹, or —C(═O)CH₂NHCH₂R¹⁹;
R¹⁵ is selected from —OR¹⁶, —N(R¹⁶)₂, —C(═O)OR¹⁶, or —C(═O)N(R¹⁶)₂;
   each R¹⁶ is independently H, —C₁-C₆ alkyl, —C(═O)—(CH₂)ᵥR¹⁹, —C(═O)CH₂NHCH₂R¹⁹, or a saccharide or derivative thereof;
   R¹⁹ is 4-iodophenylene, 4-methylphenylene, or 3-fluoro-4-methylphenylene;
   y is 0, 1, 2, 3, 4, 5, 6, 7, 8, 9, or 10;
   x is an integer from 1 and 25; and
   v is 1,2,3, or 4;
wherein any free —NH— of a peptide bond is optionally independently substituted with —CH₃ or —CH₂CH₃; and
wherein any alpha position of an amino acid is optionally independently substituted with —CH₃ or —CH₂CH₃.

Embodiment 85. The compound of embodiment 84, or a pharmaceutically acceptable salt thereof, wherein R¹ is

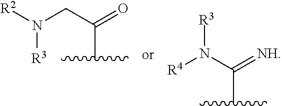

Embodiment 86. The compound of embodiment 84 or 85, or a pharmaceutically acceptable salt thereof, wherein R is H and R³ is H or C₁-C₄ alkyl.

Embodiment 87. The compound of embodiment 84 or 85, or a pharmaceutically acceptable salt thereof, wherein R³ is H and R⁴ is H or C₁-C₄ alkyl.

Embodiment 88. The compound of embodiment 84 or 85, or a pharmaceutically acceptable salt thereof, wherein R² is —(CHR⁶)ₙ-aryl.

Embodiment 89. The compound of embodiment 88, or a pharmaceutically acceptable salt thereof, wherein n is 1.

Embodiment 90. The compound of embodiment 88 or 89, or a pharmaceutically acceptable salt thereof, wherein R⁶ is H.

Embodiment 91. The compound of embodiment 84 or 85, or a pharmaceutically acceptable salt thereof, wherein R¹ is

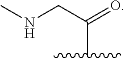 [SMILES: *C(=O)CNC]

Embodiment 92. The compound of embodiment 84 or 85, or a pharmaceutically acceptable salt thereof, wherein R¹ is

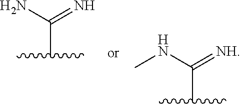

Embodiment 93. The compound of embodiment 84 or 85, or a pharmaceutically acceptable salt thereof, wherein R¹ is

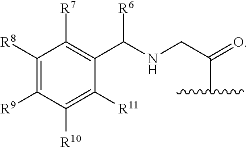

Embodiment 94. The compound of embodiment 84 or 85, or a pharmaceutically acceptable salt thereof, wherein R¹ is

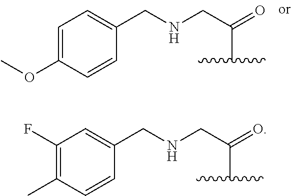 [SMILES: *C(=O)CNCc1ccc(OC)cc1 ; *C(=O)CNCc1ccc(C)c(F)c1]

Embodiment 95 The compound of embodiment 93, or a pharmaceutically acceptable salt thereof, wherein R⁷, R⁸, R⁹, R¹⁰, and R¹¹ are each independently selected from H, F, Cl, Br, L, —OH, —O—C₁-C₄ alkyl, —NH₂, or —C₁-C₆ alkyl.

Embodiment 96. The compound of embodiment 93, or a pharmaceutically acceptable salt thereof, wherein R⁷, R⁸, R⁹, R¹⁰, and R¹¹ are each independently selected from H, F, Cl, Br, I, —OH, —OCH₃, ˜NH₂, or —CH₃.

Embodiment 97. The compound of embodiment 93, or a pharmaceutically acceptable salt thereof, wherein R⁸ is F and R⁹ is CH₃.

Embodiment 98. The compound of any one of embodiments 84-97, or a pharmaceutically acceptable salt thereof, wherein: X¹ is tyrosine (Tyr).

Embodiment 99. The compound of any one of embodiments 84-97, or a pharmaceutically acceptable salt thereof, wherein: X² is absent.

Embodiment 100. The compound of any one of embodiments 84-97, or a pharmaceutically acceptable salt thereof, wherein: X³ is 3-(2-naphthyl)alanine (β-Nal) or tryptophan (Trp).

Embodiment 101. The compound of any one of embodiments 84-97, or a pharmaceutically acceptable salt thereof, wherein: X⁴ is asparagine (Asn).

Embodiment 102. The compound of any one of embodiments 84-97, or a pharmaceutically acceptable salt thereof, wherein: X⁵ is threonine (Thr).

Embodiment 103. The compound of any one of embodiments 84-97, or a pharmaceutically acceptable salt thereof, wherein: X⁶ is phenylalanine (Phe) or cyclohexylalanine (Cha).

Embodiment 104. The compound of any one of embodiments 84-97, or a pharmaceutically acceptable salt thereof, wherein: X⁷ is azaglycine (aza-gly).

Embodiment 105. The compound of any one of embodiments 84-97, or a pharmaceutically acceptable salt thereof, wherein: X⁸ is leucine (Leu).

Embodiment 106. The compound of any one of embodiments 84-97, or a pharmaceutically acceptable salt thereof, wherein: X¹⁰ is tryptophan (Trp), 1-methyltryptophan (1MT), tyrosine (Tyr), phenylalanine (Phe), 4-cyano phenylalanine (Phe(4-CN)), 3-(4-pyridyl)alanine (4-Pal), or leucine (Leu).

Embodiment 107. The compound of any one of embodiments 84-97, or a pharmaceutically acceptable salt thereof, wherein: X¹⁰ is tryptophan (Trp), tyrosine (Tyr), or phenylalanine (Phe).

Embodiment 108. The compound of any one of embodiments 84-97, or a pharmaceutically acceptable salt thereof, wherein:
X¹ is absent, tyrosine (Tyr), or 3-(3-pyridyl)alanine (3-Pal));
X² is absent, asparagine (Asn), glutamine (Gln), serine (Ser), D-histidine (His), or phenylalanine (Phe);
X³ is absent, tryptophan (Trp), isoleucine (Ile), 3-(4-pyridyl)alanine (4-Pal), lysine (Lys), aspartic acid (Asp), glutamnic acid (Glu), glycine (Gly), alanine (Ala), cyclohexylalanine (Cha), (Hyp), biphenylalanine (Bip); 4-benzoylphenylalanine (Bpa), or 3-(9-anthryl)-alanine (AAP);
X⁴ is absent, asparagine (Asn), or glutamine (Gln); and
X⁵ is absent, serine (Ser), threonine (Thr), glycine (Gly), or alanine (Ala).

Embodiment 109. The compound of any one of embodiments 84-97, or a pharmaceutically acceptable salt thereof, wherein: X¹ is D-tyrosine (D-Tyr);
X² is absent;
X³ is D-tryptophan (D-Trp), biphenylalanine (Bip); 4-benzoylphenylalanine (Bpa), or 3-(9-anthryl)-alanine (AAP);
X⁴ is asparagine (Asn); and
X⁵ is serine (Ser) or threonine (Thr).

Embodiment 110. The compound of any one of embodiments 84-97, or a pharmaceutically acceptable salt thereof, wherein:
X⁶ is phenylalanine (Phe), 3-fluorophenylalanine (3-F-Phe), biphenylalanine (Bip), or cyclohexylalanine (Cha);
X⁷ is glycine (Gly) or aza-glycine (aza-Gly); and
X⁸ is leucine (Leu) or norvaline (Nva).

Embodiment 111. The compound of any one of embodiments 84-97, or a pharmaceutically acceptable salt thereof, wherein:
X⁶ is phenylalanine (Phe) or cyclohexylalanine (Cha);
X⁷ is aza-glycine (aza-Gly); and
X⁸ is leucine (Leu).

Embodiment 112. The compound of any one of embodiments 84-97, or a pharmaceutically acceptable salt thereof, wherein:
X⁸ is Leu, Nva, lie, Hala, or Phe; and
X¹⁰ is Trp, 1MT, Tyr, 4-Pal, Phe(4-CN), or Phe.

Embodiment 113. The compound of any one of embodiments 84-97, or a pharmaceutically acceptable salt thereof, wherein:

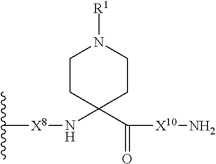

is

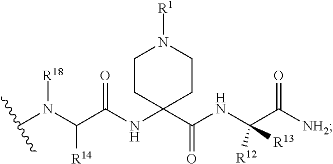

wherein,
R¹⁸ is H or —CH₃;
R¹² is

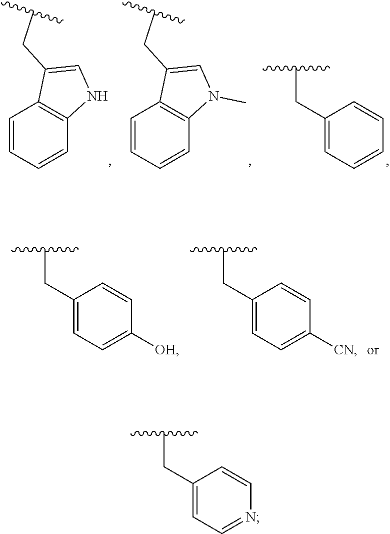

R¹³ is H or —CH₃; and
R¹⁴ is

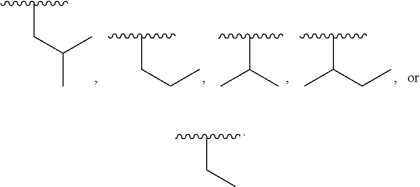

Embodiment 114. The compound of any one of embodiments 84-113, or a pharmaceutically acceptable salt thereof, wherein the N-terminal amino acid or the compound of Formula (II) is optionally substituted with —C(═O)—C₁-C₂alkyl, —C(═O)—(CH₂CH₂O)_{y}—CH₂CH₂—R¹⁵, —C₁-C₂₀alkyl, N-hexadecanoyl-Glu, —C₄-C₂₀ polyethylene glycol, a saccharide, —R¹⁶, —C(═O)—(CH₂CH₂O)ₓ—CH₃, —C(═O)—(CH₂CH₂O)ₓ—H, —C(═O)—CH₂CH₂CH(COOH)—R¹⁵, —C(═O)—(CH₂CH₂R¹⁹, or —C(═O)CH₂NHCH₂R¹⁹;
R¹⁵ is selected from —OR¹⁶, —N(R¹⁶)₂, —C(═O)OR¹⁶, or —C(═O)N(R¹⁶)₂;
each R¹⁶ is independently H, —C₁-C₆ alkyl, —C(═O))—(CH₂)ᵥR¹⁹, —C(═O) CH₂NHCH₂R¹⁹, or a saccharide or derivative thereof;
   R¹⁹ is 4-iodophenylene, 4-methylphenylene, or 3-fluoro-4-methylphenylene;
y is 0, 1, 2, 3, 4, 5, 6, 7, 8, 9, or 10;
x is an integer from 1 and 25; and
v is 1, 2, 3, or 4.

Embodiment 115. The compound of any one of embodiments 84-113, or a pharmaceutically acceptable salt thereof, wherein the N-terminal amino acid or the compound of Formula (II) is optionally substituted with —C(═O)—C₁-C₁₂ alkyl.

Embodiment 116. The compound of embodiment 115, or a pharmaceutically acceptable salt thereof, wherein the N-terminal amino acid or the compound of Formula (II) is optionally substituted with

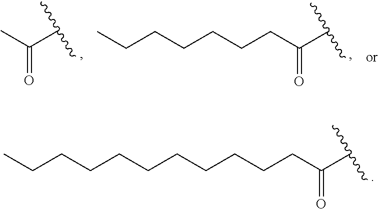

Embodiment 117. The compound of any one of embodiments 84-113, or a pharmaceutically acceptable salt thereof, wherein the N-terminal amino acid or the compound of Formula (II) is optionally substituted with —C(═O)—(CH₂CH₂O)_{y}—CH₂CH₂—R¹⁵.

Embodiment 118. The compound of embodiment 117, wherein y is 2.

Embodiment 119. The compound of embodiment 117 or 118, wherein R¹⁵ is —N(R¹⁶)₂ and both R¹⁶ are H.

Embodiment 120. The compound of embodiment 117 or 118, wherein R-⁵ is —N(R¹⁶)₂, one R¹⁶ is H and the other R¹⁶ is —C(═O)—(CH₂)ᵥR¹⁹.

Embodiment 121. The compound of any one of embodiments 84-113, or a pharmaceutically acceptable salt thereof, wherein the N-terminal amino acid or the compound of Formula (II) is optionally substituted with —R¹⁶.

Embodiment 122. The compound of embodiment 123, wherein R¹⁶ is —C(═O)—(CH₂)ᵥR¹⁹.

Embodiment 123. The compound of embodiment 120 or 122, wherein v is 2 or 3.

Embodiment 124. The compound ofany one of embodiments 120, 122, or 123 wherein R¹⁹ is 4-iodophenylene or 4-methylphenylene.

Embodiment 125. The compound of any one of embodiments 84-113, or a pharmaceutically acceptable salt thereof, wherein the N-terminal amino acid or the compound of Formula (II) is optionally substituted with

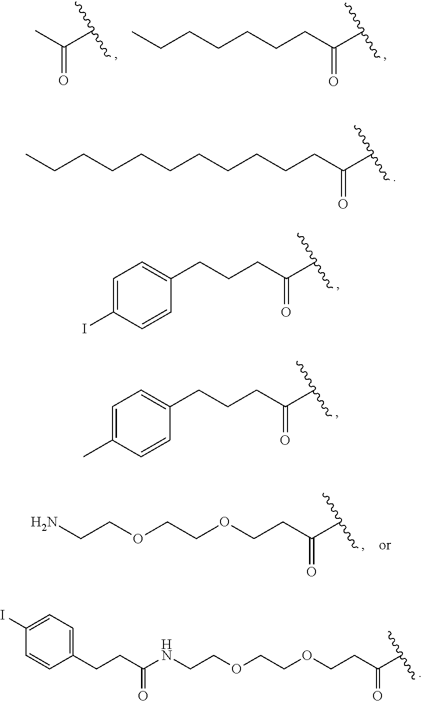

Embodiment 126. The compound of embodiment 84, or a pharmaceutically acceptable salt thereof, wherein the compound of Formula (II) has one of the following structures, or a pharmaceutically acceptable salt thereof:

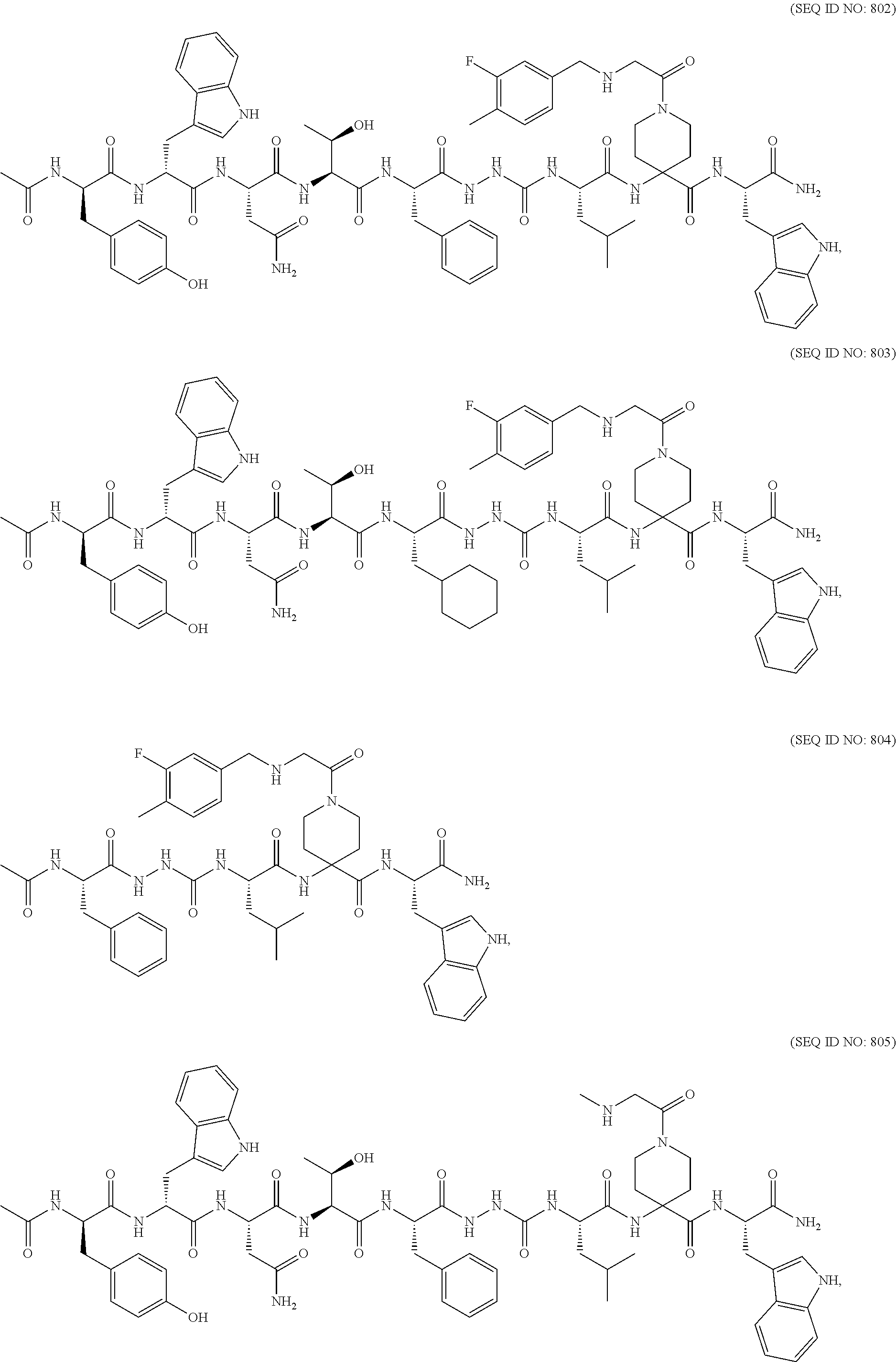

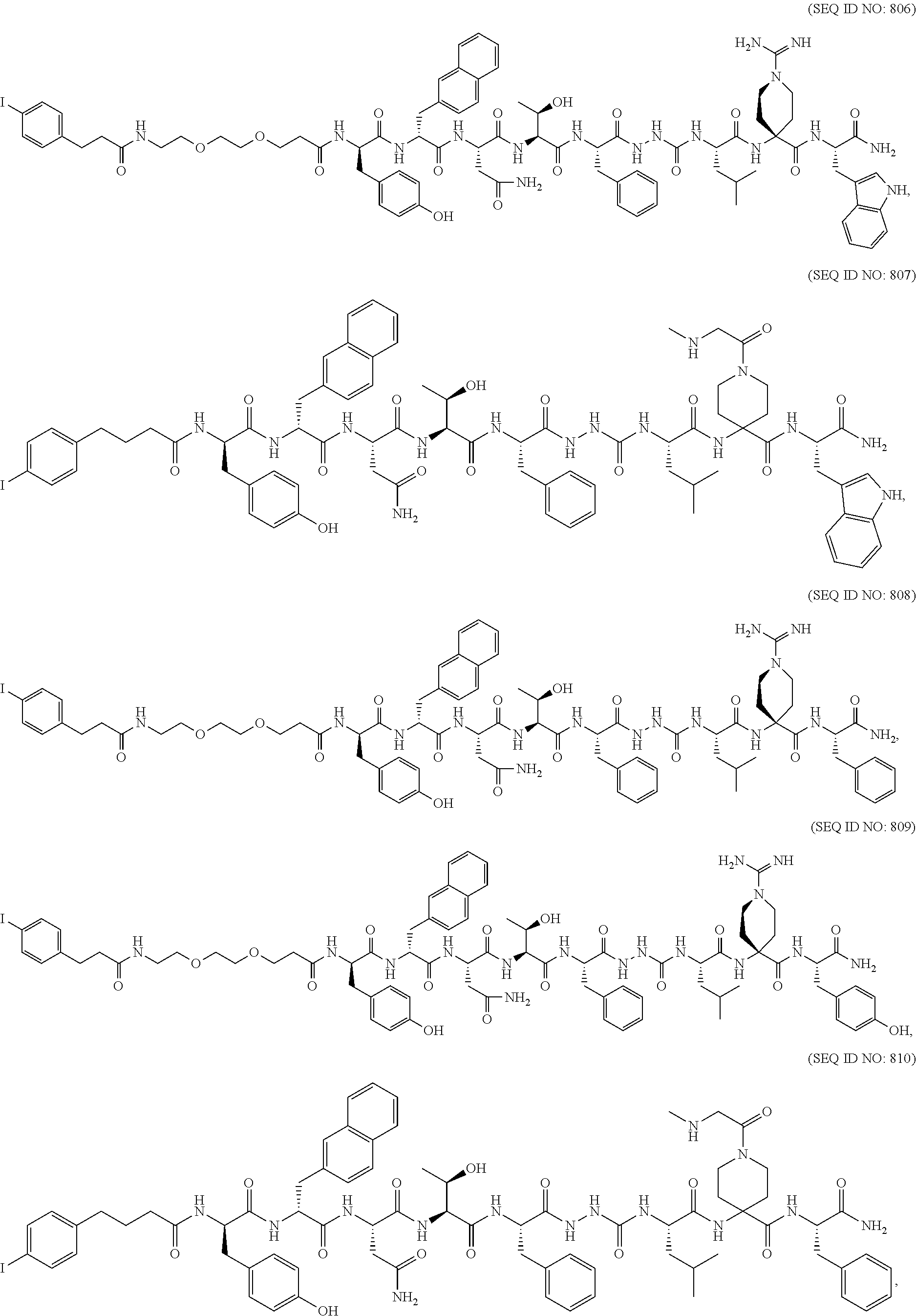

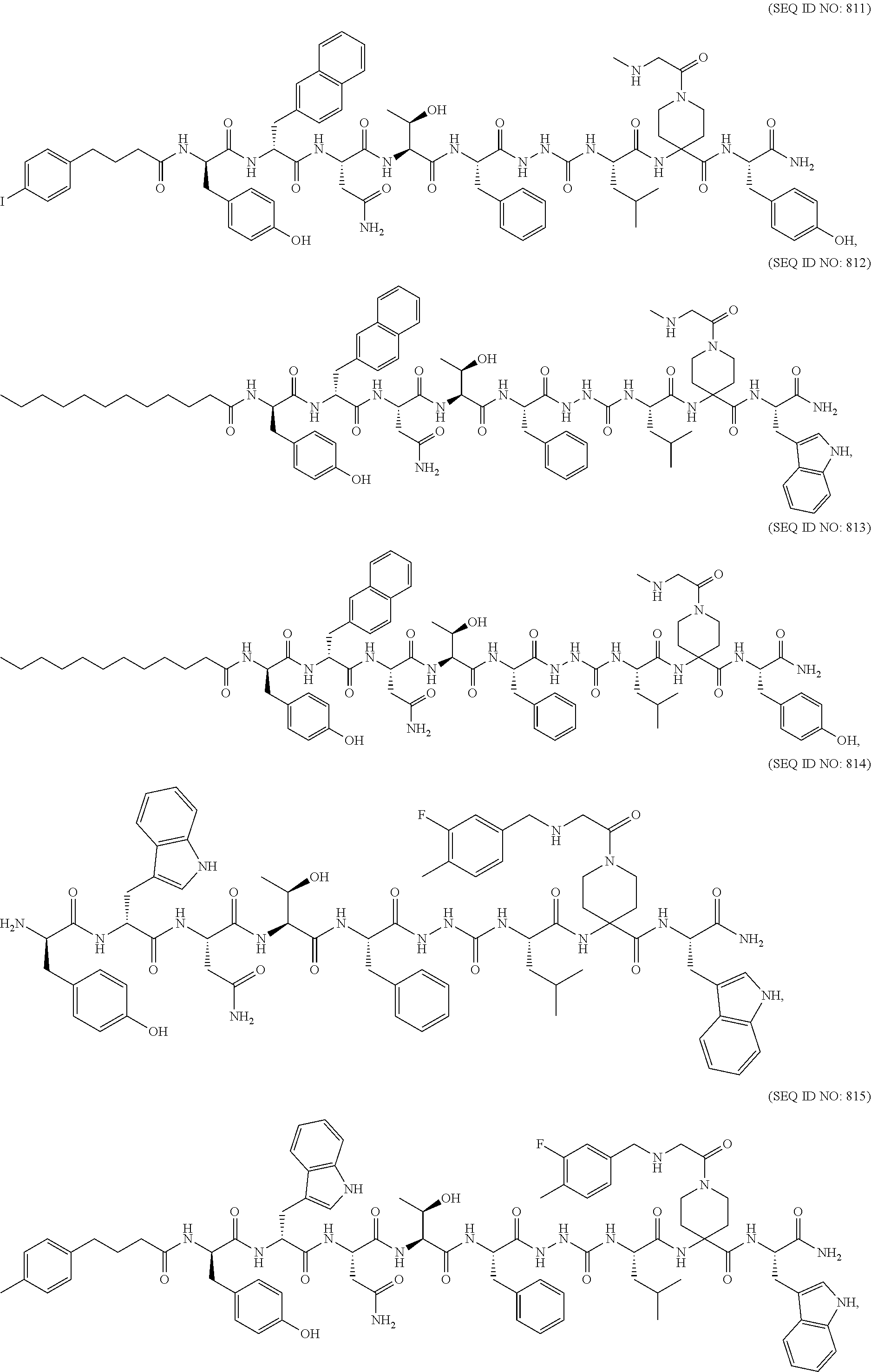

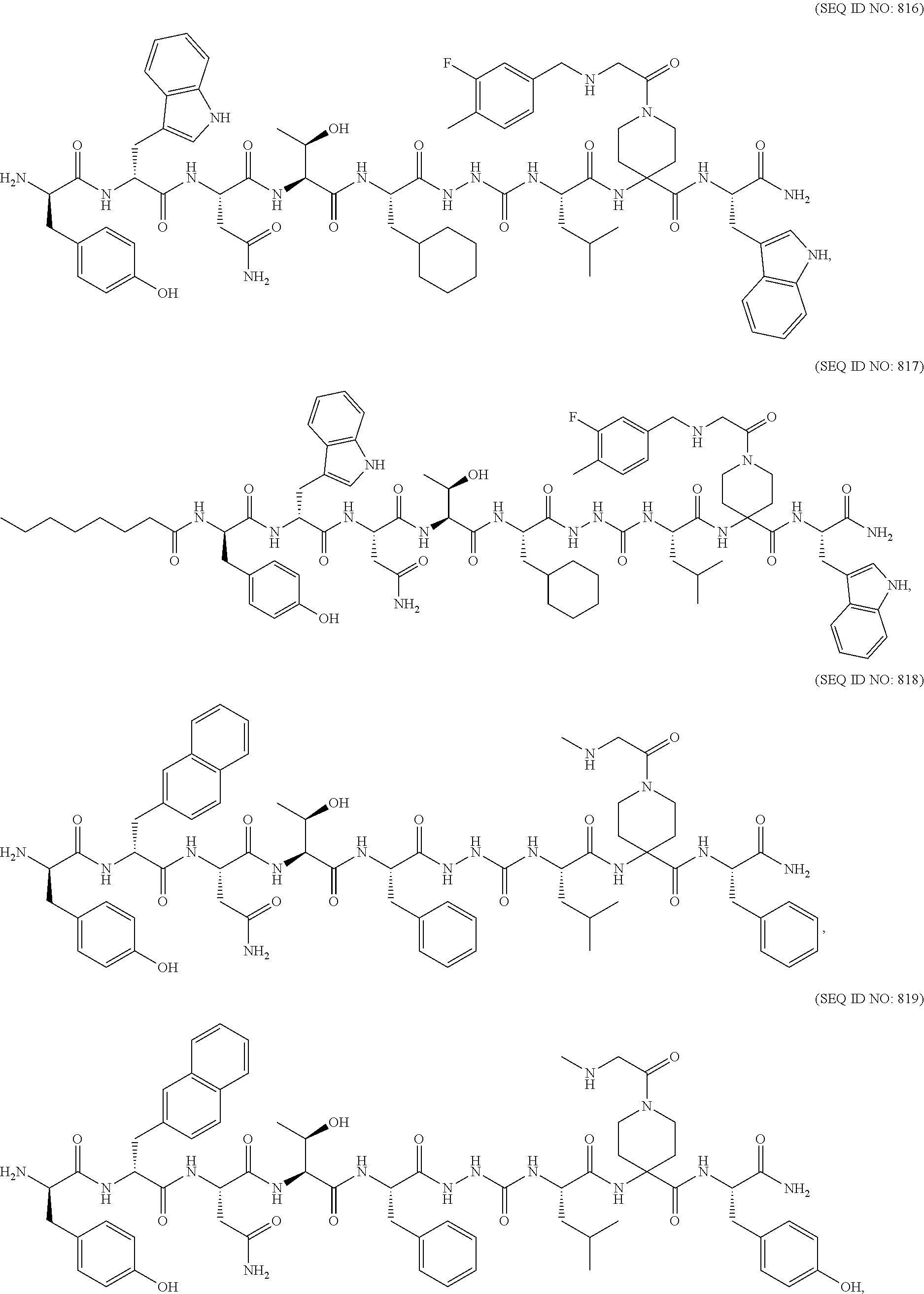

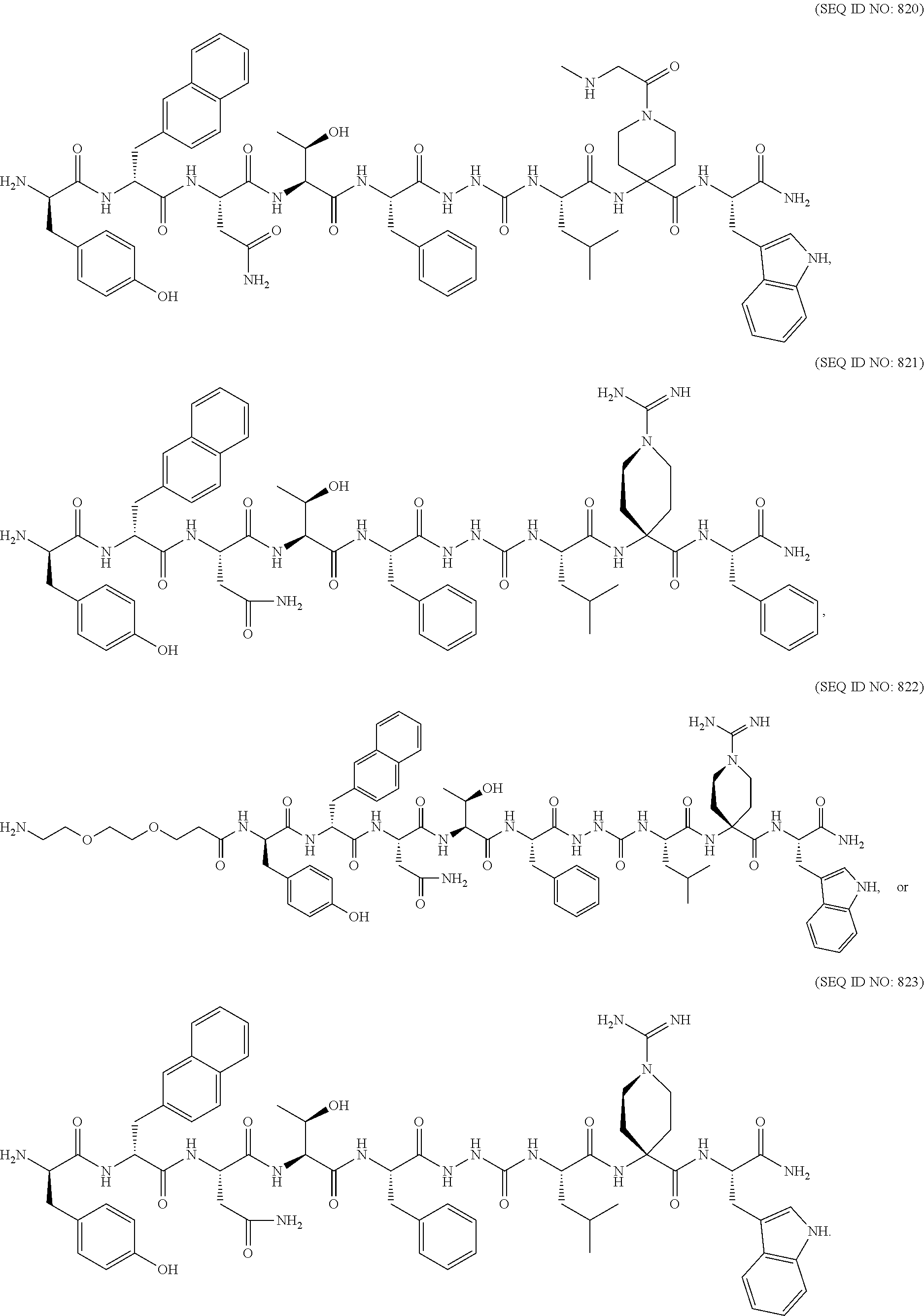

Embodiment 127. A pharmaceutical composition comprising a compound of any one of embodiments 84-126, or a pharmaceutically acceptable salt thereof, and at least one pharmaceutically acceptable excipient.

Embodiment 128. The pharmaceutical composition of embodiment 127, wherein the pharmaceutical composition is formulated for administration to a mammal by oral administration.

Embodiment 129. A method for the treatment of an endocrine condition comprising administering to a mammal an effective amount of a compound of any one of embodiments 84-126, or a pharmaceutically acceptable salt thereof.

Embodiment 130. The method of embodiment 129, wherein the endocrine condition is polycystic ovary syndrome (PCOS).

Embodiment 131. The method of embodiment 129, wherein the endocrine condition is infertility.

Embodiment 132. A method for the treatment of cancer comprising administering to a mammal an effective amount of a compound of any one of embodiments 84-126, or a pharmaceutically acceptable salt thereof.

Embodiment 133. The method of embodiment 117, wherein cancer is prostate cancer or breast cancer.

Embodiment 134. A method for the treatment of infertility comprising administering to a mammal an effective amount of a compound of any one of embodiments 84-126, or a pharmaceutically acceptable salt thereof.

### EXAMPLES

The following examples are provided for illustrative purposes only and not to limit the scope of the claims provided herein.

### Abbreviations

ACN or MeCN or CH₃CN acetonitrile; BBr₃: boron tribromide; brine: saturated NaCl solution;
BOP: Benzotriazole-1-yl-oxy-tris-(dimethylamino)-phosphonium hexafluorophosphate;
CDT: 1, 1′-carbonyl-di-(1,2,4-triazole); CTC resin: chlorotrityl chloride resin;
DBAD: Di-tert-butyl azodicarboxylate; DCC: dicyclohexylcarbodiimide;
DCM: dichloromethane; Dde-OH: 2-Acetyldimedone;
DHP resin: dihydropyranylmethoxymethyl resin;
DODT: 3,6-dioxa-1,8-octanedithiol; DIEA or DIPEA: N,N-diisopropylethylamine;
DMF: dimethylformamide; DMSO: dimethyl sulfoxide;
DOTA: 2,2′,2″,2″′-(1,4,7,10-tetraazacyclododecane-1,4,7,10-tetrayl)tetraacetic acid or 1,4,7,10-tetraazacyclododecane-1,4,7,10-tetraacetic acid;
EDC: (1-ethyl-3-(3-dimethylaminopropyl)carbodiimide hydrochloride);
EtOAc or EA: ethyl acetate; Fu catalyst complex: tris(dibenzylideneacetone)dipalladium(0) tri-tert-butylphosphonium tetrafluoroborate
Fmoc: fluorenylmethoxycarbonyl; Fmoc-OSu: N-(9-Fluorenylmethoxycarbonyloxy)succinimide
HATU: 1-[Bis(dimethylamino)methylene]-1H-1,2,3-triazolo[4,5-b]pyridinium 3-oxide hexafluorophosphate; HCl: hydrochloric acid or hydrochloride; Hex: hexanes; H₂O: water;
HBTU: N,N,N′,N′-Tetramethyl-O-(1H-benzotriazol-1-yl)uronium hexafluorophosphate;
HOBt: hydroxybenzotriazole; HMBA: hydroxymethyl benzoic acid resin;
HPLC: high-performance liquid chromatography; InCl₃: indium trichloride;
K₂CO₃: potassium carbonate; KOAc: potassium acetate; KOt-Bu: potassium t-butoxide
K₃PO₄: potassium phosphate; LCMS: Liquid chromatography-mass spectrometry;
LuCl₃: lutetium (III) chloride; MBH A resin: methylbenzhydryl amine resin;
MeOH: methanol; MPLC: Medium pressure liquid chromatography;
MS: mass spectrometry; NaH: sodium hydride; NaHCO₃: sodium bicarbonate;
NalO₄: sodium periodate; NaN[(CH₃)₃Si]₂: sodium bis(trimethylsilyl)amide
NaOAc: sodium acetate; Na₂SO₄: sodium sulfate; NMP: N-Methyl-2-pyrrolidone;
Pd(dppf)Cl₂: [1, 1′-Bis(diphenylphosphino)ferrocene]dichloropalladium(II);
Pd(DTBPF)Cl₂: [1, 1′-Bis(di-tert-butylphosphino)ferrocene]dichloropalladium(II);
PE: petroleum ether; PPh₃: triphenyl phosphine; RuCl₃: ruthenium(III) chloride;
Prep-HPLC: preparative high-performance liquid chromatography;
RP-HPLC: reversed-phase high-performance liquid chromatography;
SPPS: solid-phase peptide synthesis; TFA: trifluoroacetic acid; THF: tetrahydrofuran;
TIS: triisopropylsilane; rt: room temperature; hrs: hours; h or Ir: hour; min: minute;
mg: milligrams; kg: kilograms; mL or ml: milliliter;
Eq: equivalents; mmol: millimole; mol: moles; UV: ultraviolet; v/v: volume/volume.

Reagents were obtained from commercial suppliers and used without further purification, unless otherwise noted.

Reactions were carried out in Syro II (Biotage) and/or manual shaker using Fmoc chemistry, unless otherwise noted.

N^{α}-Fmoc-Rink amide resin purchased from Novabiochem (100-200 mesh, 0.62 mmol/g loading) was used for SPPS.

MPLC purifications were performed with a Orinedia preparative HPLC (BRIX 2802) on silica gel columns.

RP-HPLC purifications were performed on a Waters preparative HPLC (2767) at room temperature with reverse-phase C18 columns (X Select CSH C18 5 μm, 30 mm×150 mm).

HPLC solvents are H₂O containing 0.05% trifluoroacetic acid (mobile phase A) and acetonitrile (mobile phase B).

HPLC analysis was carried out with a Shimadzu LCMS (2020 series) containing a binary pump (LC-20AD), micro vacuum degasser, auto sampler (SIL-20AC HT), thermostat column compartment (CTO-20A), variable wavelength detector (SPD-M20A). HPLC data was analyzed using Lab Solutions software from the Shimadzu LCMS (2020 series). A Kinetex EVO column (2.6 μm, 100 Å, 4.6×100 mm) was used with a flow rate of 1.0 mL/min.

LCMS analysis was carried out with a Shimadzu LCMS (2020 series) containing a binary pump (LC-20ADXR), micro vacuum degasser, auto sampler (SIL-20AC XR), thermostat column compartment (CTO-20AC), variable wavelength detector (SPD-M20A). LCMS data was analyzed using Lab Solutions software from Agilent Technologies. An Ascentis Express C18 column (2.7 μm, 3.0×50 mm) was used with a flow rate of 1.5 mL/min.

¹H NMR spectra were recorded using an AVANCE III HD 300 MHz, AVANCE NEO 400 MHz, or Bruker 300 MHz or 400 MHz. Chemical shifts are reported in δ (ppm) relative to TMS₄Si (in CDCl₃) as internal standard using Bruker TopSpin software unless otherwise noted.

A peptide of the present disclosure may be prepared through known methods, including procedures referenced in “Design and synthesis of downsized metastin (45-54) analogs with maintenance of high GPR54 activity” Niida et al., Bioorganic & Medicinal Chemistry Letters 16: 134-137 (2006); “A synthetic kisspeptin analog that triggers ovulation and advances puberty” Decourt et al., Scientific Reports 6: 26908 (2016); “A kisspeptin-10 analog with greater in vivo bioactivity than kisspeptin-10” Curtis et al., American Journal of Physiology—Endocrinology and Metabolism 298: E296-E3303 (2010); “RFamide Peptides: Structure, Function, Mechanisms and Pharmaceutical Potential” Pharmaceuticals 4: 1248-1280 (2011). Findeisen et al.; “Serum stability of selected decapeptide agonists of KISS1R using psuedopeptides” Asami et al., Bioorganic & Medicinal Chemistry Letters 22: 6391-6396 (2012); “Rational design of triazololipopeptides analogs of kisspeptin inducing a long-lasting increase of gonadotropins” Beltramo et al., Journal of Medicinal Chemistry 58: 3459-3470 (2015); “Design, synthesis, and biological evaluation of novel investigational nonapeptide KISS1R agonists with testosterone-suppressive activity” Asami et al., Journal of Medicinal Chemistry 56: 8298-8307 (2013); “Trypsin resistance of a decapeptide KISS1R agonist containing an N^{ω}-methylarginine substitution” Asami et al., Bioorganic & Medicinal Chemistry Letters 22: 6328-6332 (2012); U.S. Pat. No. 9,884,891; PCT Appl. No. PCT/1EP2014/051886; U.S. Pat. Nos. 8,404,643; 8,778,871; 8,361,968; U.S. application Ser. No. 12/989,346; U.S. Pat. No. 6,800,611; JP Appl. No. JP2003433643.

### Solid-Phase Peptide Synthesis (SPPS)

### Procedure A: General Procedure for SPPS

### Procedure A-1: Resin Swelling and Attachment of First Amino Acid on Resin (CTC Resin)

2-Chlorotrityl chloride resin (1.1 mmol/g) and DCM (10 mL/g resin) were added into a sealed tube at room temperature under nitrogen. The mixture was swollen for 15 min at room temperature under nitrogen. The resin was washed with DCM (3×100 mL). The appropriate amino acid (1.0 eq.), DIEA (1.0 eq.), and DCM (10 mL/resin) were added to the mixture. The mixture was agitated for 5 min at room temperature under nitrogen. DIEA was added (1.5 eq.), and the mixture was agitated for another 60 min. MeOH (I-PLC grade, 0.8 mL/g resin) was added to endcap any remaining reactive trityl groups. The resin was filtered and washed twice with DCM (10 mL/g resin), twice with DMF, and three times with MeOH. The resin was dried under vacuum and the loading was calculated by weight gain.

### Procedure A-2: Resin Swelling (Rink Amide Resin)

The resin (100 mg/tube) was swollen with NMP (1 mL/tube) for 1-5 minutes at room temperature under nitrogen. The resin was washed four times with NMP (1 mL/tube).

### Procedure A-3: Fmoc Deprotection

The resin (100 mg/tube) was treated with 20% piperidine in NMP (1 mL) for 20 minutes at room temperature under nitrogen. The resin was washed four times with NMP (1 mL/tube).

### Procedure A-4: HATU Coupling

The resin (100 mg/tube) was treated with a mixture of amino acid (4.0 eq.), HATU (4.0 eq.), and DIEA (8.0 eq.) in NMP for 45 min at 30° C. under nitrogen. The resin was washed four times with NMP (1 mL/tube).

### Procedure A-5: Resin Capping

The resin (100 mg/tube) was capped with a solution of Ac₂O/DIEA/NMP (31.5:8:5:160 v/v/v) for 1 hour at room temperature under nitrogen. The resin was washed four times with NMP (1 mL/tube).

### Procedure A-6: Cleavage and Purification (CTC Resin)

The crude peptide was cleaved from the resin with a solution of 1,1,1,3,3,3-hexafluoropropan-2-ol/DCM (1:4 v/v) for 30 min at room temperature. The crude product was purified by prep-HPLC.

### Procedure A-7: Cleavage and Purification (Rink Amide Resin)

The crude peptide was cleaved from the resin with a solution of TFA/H₂O/TIS/DODT (37:1:1:1 v/v/v/v) for 2 h at room temperature. The mixture was filtered, and the filtrate was concentrated under reduced pressure. The residue was precipitated with cold ether, then the precipitate was centrifuged. The crude product was purified by prep-HPLC and dried by lyophilization.

### Procedure B: Synthesis of (S)-2-(4-((S)-1-((((9H-fluoren-9-yl)methoxy)carbonyl)amino)-2-phenylethyl)-1H-1,2,3-triazol-t-yl)-4-methylpentanoic acid

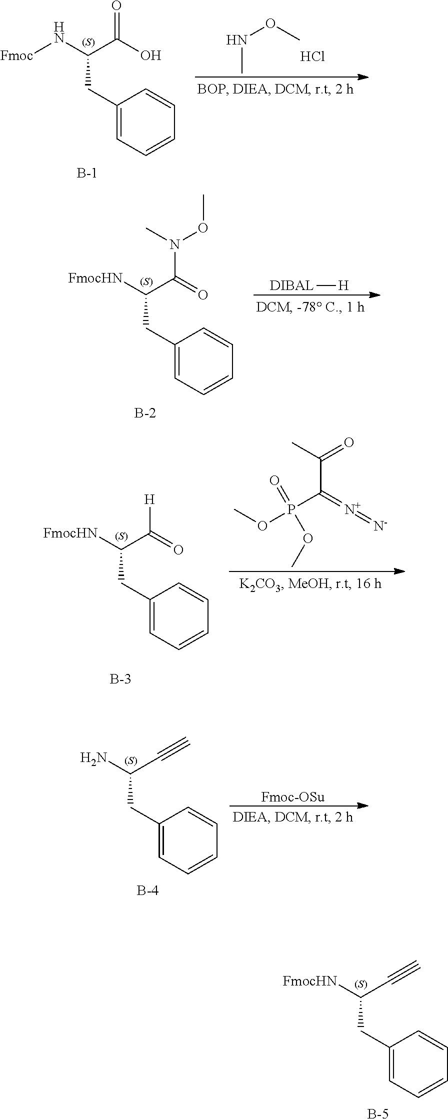

Into a 250-mL round bottom flask were added B-1 (10 g, 26 mmol, 1.0 eq), BOP (14 g, 32 mmol, 1.2 eq), DIEA (10 g, 13 mL, 77 mmol, 3.0 eq) and DCM (100 mL). The reaction mixture was stirred at 20° C. for 10 minutes, then N,O-dimethylhydroxylamine hydrochloride (3.8 g, 39 mmol, 1.5 eq) was added. The reaction mixture was stirred at 25° C. for an additional 2 h. The mixture was diluted with water (500 ml), extracted with EtOAc (3×200 mL), and the combined organic layers were washed with brine (200 mL), dried over anhydrous Na₂SO₄, filtered and concentrated under reduced pressure. The residue was purified by MPLC using a silica gel column (330 g) with a PE/EtOAc mobile phase (0% to 12% EtOAc in 25 min, 100 mL/min flow rate, UV 254 nm). The collected fractions were concentrated under reduced pressure to afford B-2 (9.2 g, 21 mmol, 83%) as a yellow solid. LCMS: (ESI, m/z): [M+Na]⁺=453.3.

To a solution of B-2 (9.2 g, 21 mmol, 1.0 eq) in DCM (90 mL) was added diisobutylaluminum hydride (40 mL, 2 M in toluene, 80 mmol. 3.7 eq) dropwise at −78° C. over a period of 10 minutes under a nitrogen atmosphere. The reaction mixture was stirred at −78° C. for 1 h and quenched by dropwise addition of MeOH (100 mL). After the solvent was removed, the crude B-3 (8.0 g, 13 mmol, 60%) was carried forward without further purification.

Into a 500-mL three-neck round bottom flask was added B-3 (8.0 g, 60% Wt, 13 mmol, 1.0 eq), followed by K₂CO₃ (4.0 g, 29 mmol, 2.2 eq) and dimethyl (1-diazo-2-oxopropyl)phosphonate (3.7 g, 19 mmol, 1.5 eq). The reaction mixture was stirred at 25° C. for 16 h. The mixture was diluted with 200 mL of potassium sodium tartrate solution and stirred at 25° C. for 2 h. The mixture was extracted with DCM (3×100 mL), and the combined organic layers were washed with brine (100 mL), dried over anhydrous Na₂SO₄, and concentrated under reduced pressure to afford B-4 (6.0 g, 12 mmol, 96%) as a yellow oil, which was used without further purification.

To a mixture of B-4 (6.0 g, 12 mmol, 1.0 eq), DIEA (4.8 g, 6.5 mL, 37 mmol, 3.0 eq) and DCM (60 mL) was added Fmoc-OSu (6.3 g, 19 mmol, 1.5 eq). The reaction mixture was stirred at 20° C. for 2 h. The mixture was concentrated under reduced pressure. The residue was purified by MPLC using a silica gel column (80 g) with a PE/EtOAc mobile phase (0% to 12% EtOAc in 40 min, 80 mL/min flow rate, UV 220 nm). The collected fractions were concentrated under reduced pressure to afford B-5 (3.6 g, 9.8 mmol, 79%) as a white solid. LCMS: (ESI, m/z): [M+H]⁺=368.1. ¹H NMR (300 MHz, Chloroform-d) δ 7.80 (d, J=7.5 Hz, 2H), 7.59 (d, J=7.4 Hz, 2H), 7.43-7.38 (m, 2H), 7.39-7.21 (m, 1H), 4.98-4.90 (m, 1H), 4.82-4.76 (m, 1H), 4.56-4.38 (m, 2H), 4.23 (t, J=6.8 Hz, 1H), 3.04-2.91 (m, 2H), 2.34 (d, J=2.3 Hz, 1H).

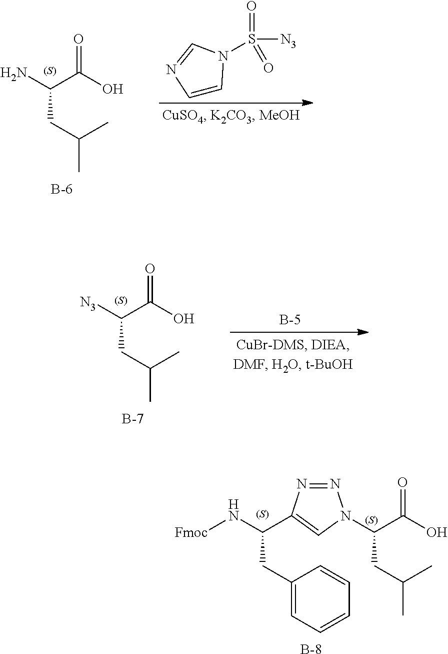

A solution of L-leucine (3 g, 22.9 mmol, 1 Eq), imidazole-1-sulfonyl azide (4.75 g, 27.4 mmol, 1.2 Eq), CuSO₄ (73.0 mg, 0.46 mmol, 0.02 Eq) and K₂CO₃ (6.32 g, 45.7 mmol, 2.0 Eq) in MeOH (120 mL) was stirred at 25° C. overnight and concentrated in vacuo. To the residue was added water (40 mL), and the mixture was acidified to pH 2 with HCl (aq.). The resulting mixture was extracted with EtOAc (4×50 mL). The combined organic layers were dried over anhydrous Na₂SO₄. After filtration, the filtrate was concentrated under reduced pressure to afford B-7 (2.9 g, 64.5%) as a green oil. Crude B-7 was used without further purification. LCMS: (ESI, m/z): [M−H]⁻=156.00

A solution of 17 (700 mg, 4.5 mmol, 1 Eq), 115 (1.72 mg, 4.7 mmol, 10.05 Eq), DIPEA (575.63 mg, 4.5 mmol, 1 Eq), CuBr-DMS (91.56 mg, 0.45 mmol, 0.1 Eq) in DMF (4.5 mL), t-BuOH (3.2 mL) and H₂O (0.9 mL) was stirred for 30 min at 25° C. The mixture was acidified to pH 2 with HCl (aq.). The resulting mixture was extracted with EtOAc (3×20 mL). The combined organic layers were dried over anhydrous Na₂SO₄. After filtration, the filtrate was concentrated under reduced pressure. The residue was purified by Prep-HPLC to afford B-8 (900 mg, 48.5%) as a white solid. LCMS: (ESI, m/z): [M+H]⁺=525.25. ¹H NMR (400 MHz, DMSO-d6) δ 0.83 (ddt, J=16.1, 9.5, 5.4 Hz, 6H), 1.08 (s, 1H), 1.92 (ddd, J=14.2, 9.5, 4.7 Hz, 1H), 2.06-2.24 (m, 1H), 2.97-3.12 (m, 1H), 3.20 (dd, J=13.8, 5.9 Hz, 1H), 4.18 (ddt, J=25.7, 13.8, 7.3 Hz, 3H), 4.95 (dt, J=9.9, 4.8 Hz, 1H1), 5.39 (dd, J=11.6, 4.2 Hz, 1H), 7.12-7.25 (m, 5H), 7.30 (td, J=7.3, 4.9 Hz, 2H), 7.41 (t, J=7.4 Hz, 2H), 7.64 (d, J=7.4 Hz, 2H), 7.90 (dd, J=15.2, 8.4 Hz, 3H), 8.00 (d, J=7.2 Hz, 1H), 13.44 (s, 1H).

### Procedure C: Synthesis of 4-((((9H-fluoren-9-yl)methoxy)carbonyl)amino)-1-(4-(p-tolyl)butanoyl)piperidine-4-carboxylic acid

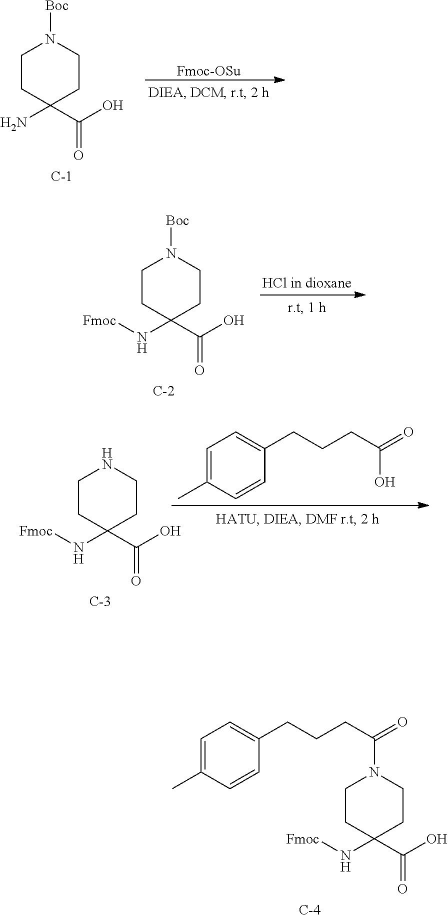

To a mixture of C-1 (2.0 g, 8.2 mmol, 1.0 eq), N-ethyl-V-isopropylpropan-2-amine (3.0 g, 23 mmol, 2.8 eq) and DCM (30 mL) was added (9H-fluoren-9-yl)methyl (2,5-dioxopyrrolidin-1-yl) carbonate (2.8 g, 8.3 mmol, 1.0 eq). The reaction mixture was stirred at 25° C. for 2 h. The mixture was concentrated, diluted with water (100 mL), and extracted with EtOAc (3×50 mL). The combined organic layers were washed with water (250 mL), brine (50 mL), dried over anhydrous Na₂SO₄, filtered and concentrated under reduced pressure. The residue was purified by MPLC using a silica gel column (40 g) with a PE/EtOAc mobile phase (0% to 35% EtOAc in 30 min, 40 mL/min flow rate, UV 254 nm). The collected fractions were concentrated under reduced pressure to provide C-2 (3.8 g, 7.3 mmol, 90%) as a yellow oil. LCMS: (ESI, m/z): [M+H]⁺=467.3.

To a solution of C-2 (2.0 g, 4.3 mmol, 1.0 eq) and 1,4-dioxane (15 mL) was added a 4 M HCl/dioxane solution (15 mL, 60 mmol, 14.0 eq). The reaction mixture was stirred at 25° C. for 2 h. The mixture was concentrated under reduced pressure, diluted with water (100 mL), and washed with EtOAc (3×50 mL). The aqueous phase was concentrated under reduced pressure to provide C-3 (2.8 g, 3.1 mmol, 71%) as a white solid. LCMS: (ESI, m/z): [M+H+MeCN]⁺=408.4.

Into a 100 mL. round bottom flask was added C-3 (1.5 g, 8.4 mmol, 1.2 eq), HATU (3.2 g, 8.4 mmol, 1.2 eq), DIEA (2.7 g, 3.6 mL, 21 mmol, 3.0 eq) and DMF (25 mL). The reaction mixture was stirred at 20° C. for 10 minutes. 4-((((9H-fluoren-9-yl)methoxy)carbonyl)amino)piperidine-4-carboxylic acid hydrochloride (2.8 g, 6.9 mmol, 1.0 eq) was added. The resulting reaction mixture was stirred for an additional 2 h at 25° C. The mixture was diluted with water (150 mL) and extracted with EtOAc (3×50 mL). The combined organic layers were washed with water (2×50 mL), brine (50 mL), dried over anhydrous Na₂SO₄, and concentrated under reduced pressure. The crude product was purified by MPLC using a silica gel column (80 g) with a PE/EtOAc mobile phase (0% to 85% EtOAc in 45 min, 80 mL/min flow rate, UV 254 nm). The collected fractions were concentrated under reduced pressure to afford C-4 (2.18 g, 4.14 mmol, 60%) as a white solid. LCMS: (ESI, m/z): [M+H]⁺=527.2. ¹H NMR (300 MHz, DMSO-d₆) δ 12.56 (brs, 1H), 7.90 (d, J=7.5 Hz, 2H), 7.78-7.70 (m, 3H), 7.48-7.23 (m, 4H), 7.08 (s, 4H), 4.3-4.18 (m, 3H), 4.11-3.99 (m, 1H), 3.68-3.58 (m, 1H), 3.25-2.15 (m, 1H), 2.96-2.82 (m, 1H), 2.60-2.52 (m, 2H), 2.31-2.20 (m, 5H), 2.00-1.82 (m, 2H), 1.76-1.58 (m, 4H).

### Procedure D: Synthesis of (S)-2-((2-(((9H-fluoren-9-yl)methoxy)carbonyl)amino)ethyl)thio)-4-methylpentanoic acid

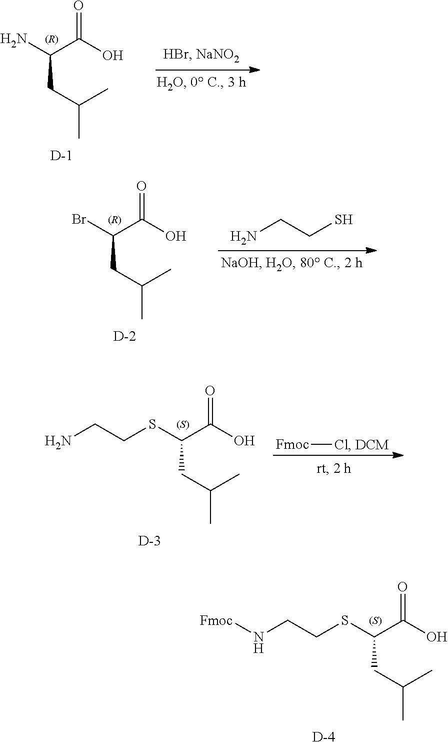

To a mixture of D-1 (6.0 g, 46 mmol, 1.0 eq) in 1420 (60 mL) were added an aqueous HBr solution (22 mL, 48 wt %, 0.19 mol, 4.3 eq), a solution of sodium nitrite (4.1 g, 2.6 mL, 59 mmol, 1.3 eq) in H₂O (40 mL) at 0° C. The reaction mixture was stirred at 0° C. for 1 hour and an additional 3 h at 25° C. The mixture was extracted with EtOAc (3×50 mL) and the combined organic layers were washed with brine (100 mL), dried over anhydrous Na₂SO₄, and concentrated under reduced pressure to afford D-2 (4.6 g, 24 mmol, 52%) as a brown crude oil, which was used without further purification. LCMS: (ESI, m/z): [M−H]⁻=193.0, 195.0.

Into a 250-mL round bottom flask were added crude D-2 (4.6 g, 1 Eq, 24 mmol), 2-aminoethane-1-thiol (1.9 g, 1.0 Eq, 25 mmol), NaOH (1.9 g, 2.0 Eq, 48 mmol) and H₂O (25 mL). The reaction mixture was stirred at 80° C. for 2 h. The crude D-3 (4.5 g, 9.6 mmol, 41%) was used directly without further purification. LCMS: (ESI, m/z): [M−H]⁻=190.1.

To a solution of crude D-3 (4.5 g, 41 wt. %, 9.6 mmol, 1.0 eq) in DCM (40 mL) was added (9H-fluoren-9-yl)methyl carbonochloridate (3.5 g, 1.4 Eq, 14 mmol) and DCM (40 mL). The reaction mixture was stirred at 20° C. for 2 h and diluted with water (60 ml). The mixture was extracted with DCM (3×50 mL), and the combined organic layers were washed with brine (50 mL), dried over anhydrous Na₂SO₄, and concentrated under reduced pressure. The residue was purified by MPLC using a silica gel column (120 g) with a PE/EtOAc mobile phase (0% to 75% EtOAc in 40 min, flow rate 100 mL/min, LV 254 nm). The collected fractions were concentrated under reduced pressure and dried by lyophilization to afford D-4 (1.3 g, 3.1 mmol, 33%) as a yellow solid. LCMS: (ESI, m/z): [M+H]⁺=414.1. ¹H NMR (300 MHz, DMSO-d₆) δ 12.53 (s, 1H), 7.90 (d, J=7.4 Hz, 2H), 7.69 (d, J=7.4 Hz, 2H), 7.49-7.28 (n. 51H), 4.35-4.17 (m, 3H), 3.29-3.12 (m, 3H), 2.70-2.55 (m, 21), 1.75-1.55 (m, 2H), 1.49-1.35 (m, 1H), 0.99-0.82 (m, 6H).

### Procedure E: Synthesis of 1-((((9H-fluoren-9-yl)methoxy)carbonyl)amino)-4-((tert-butoxycarbonyl)amino)cyclohexane-1-carboxylic acid

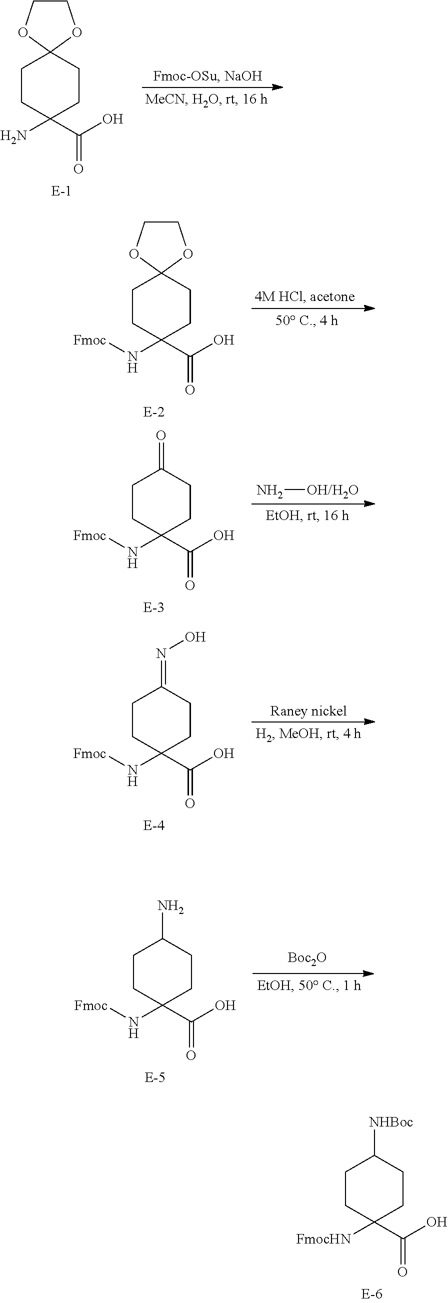

Into a 250-mL round bottom flask were added E-1 (50 g, 1 Eq, 25 mmol), NaOH (10 g, 25 mmol, 1.0 Eq), (9H-fluoren-9-yl)methyl (2,5-dioxopyrrolidin-1-yl) carbonate (8.4 g, 25 mmol, 1.0 Eq), H₂O (80 mL), and MeCN (80 mL). The reaction mixture was stirred at 25° C. for 16 h. The mixture was diluted with water (250 mL) and the pH was adjusted to 6.0 by 2N HCl solution. The mixture was extracted with EtOAc (3×250 mL), and the combined organic layers were washed with water (250 mL), brine (50 mL), dried over anhydrous Na₂SO₁, and concentrated under reduced pressure. The crude product was triturated with 200 ml of a 5:1 mixture of EtOAc:hexanes, filtered, and dried to afford E-2 (6.9 g, 16 mmol, 66%) as a white solid. LCMS: (ESI, m/z): [M+]⁺=424.1.

Into a 250-mL round bottom flask was added E-2 (6.9 g, 1 Eq, 16 mmol), acetone (70 mL) and 4M aqueous HCl solution (70 mL). The reaction mixture was stirred at 50° C. for 2 h. The mixture was concentrated under reduced pressure, filtered, and dried to afford E-3 (1-((((9H-fluoren-9-yl)methoxy)carbonyl)amino)-4-oxocyclohexane-1-carboxylic acid) (5.3 g, 14 mmol, 86%) as a white solid. LCMS: (ESI, m/z): [M+H]⁺=380.1.

Into a 250-mL round bottom flask were added E-3 (5.3 g, 1 Eq, 14 mmol), hydroxylamine (60 g, 55 mL, 50 wt. %, 0.91 mol, 65 Eq), and EtOH (55 mL). The reaction mixture was stirred at 25° C. for 16 h and concentrated. The residue was purified by MPLC The collected fractions were concentrated under reduced pressure to provide E-4 (4.4 g, 8.9 mmol, 64%) as ax white solid. LCMS: (ESI, m/z): [M+H]-=395.1.

To a solution of E-4 (4.4 g. 80 wt. % 8.9 mmol, 1.0 eq) in MeOH (100 mL) was carefully added Raney Nickel (1.4 g) under a N₂ atmosphere. The flask was evacuated and flushed with hydrogen three times, followed by flushing with hydrogen. The mixture was stirred at 25° C. for 2 h under H₂. The reaction mixture was filtered through a pad of celite and the filtrate was concentrated under reduced pressure to afford E-5 (2.8 g, 4.8 mmol, 54%) as a yellow crude oil, which was used without further purification. LCMS: (ESI, m/z): [M+H]⁺=381.2.

Into a 250-mL round bottom flask was added crude E-5 (2.9 g, 75 wt %, 5.7 mmol, 10.0 eq), di-tert-butyl dicarbonate (1.5 g, 6.9 mmol, 1.2 eq), and EtOH (40 mL). The reaction mixture was stirred at 50° C. for 2 h and concentrated in vacuo. The residue was purified by MPLC The collected fractions were concentrated under reduced pressure and dried by lyophilization to provide E-6 (1.53 g, 3.14 mmol, 55%) as a white solid. LCMS: (ESI, m/z): [M−H]⁻=479.2. ¹H NMR (300 MH z, DMSO-d₆) δ 7.89 (d, J=7.4 Hz, 2H), 7.81-7.60 (m, 2H), 7.51 (s, 1H), 7.42 (td, J=7.4, 1.4 Hz, 21), 7.40-7.31 (m, 2H), 6.85-6.78 (m, 1H), 4.22 (s, 3H), 3.23-3.18 (m, 1H), 2.20-2.06 (m, 2H), 1.68-1.52 (m, 4H), 1.48-1.30 (s, 1H).

### Procedure F: Synthesis of 4-((((9H-fluoren-9-yl)methoxy)carbonyl)amino)-1-(N-(tert-butoxycarbonyl)-N-(3-fluoro-4-methylbenzyl)glycyl)piperidine-4-carboxylic acid

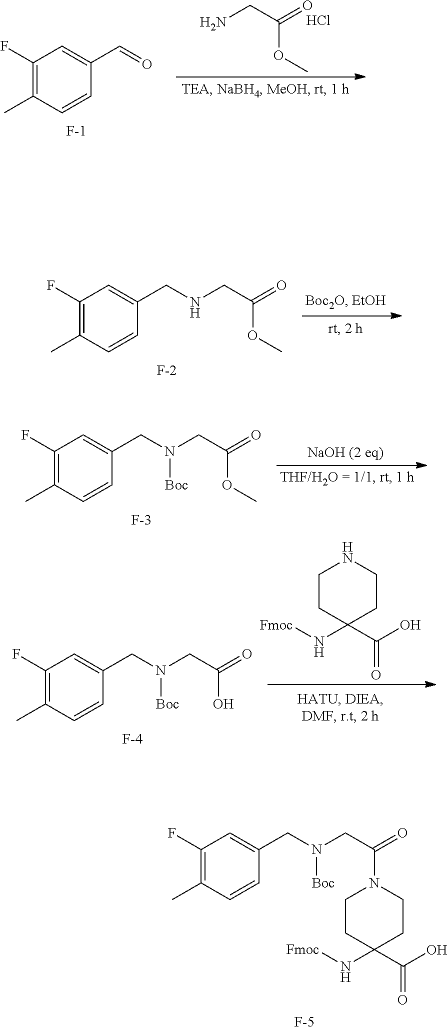

Into a 500 mL round bottom flask were added methyl glycinate hydrochloride (2 g 0.22 mol, 2.0 eq), TEA (22 g, 30 mL, 0.22 mol. 2.0 eq), F-1 (3-fluoro-4-methylbenzaldehyde) (15 g, 0.11 mol, 1.0 eq), and MeOH (150 mL). The reaction mixture was stirred at 0° C. for 30 min., then NaBH₄ (8.2 g, 0.22 mol, 2.0 eq) was added slowly under a nitrogen atmosphere at 0° C. The reaction mixture was stirred at 25° C. for an additional 1 h, and concentrated under reduced pressure. The residue was diluted with water (300 mL) and extracted with EtOAc (3×300 mL), then the combined organic layers were washed with brine (200 mL), dried over anhydrous Na₂SO₄, and concentrated under reduced pressure. The crude residue was purified by MPLC to afford F-2 (methyl (3-fluoro-4-methylbenzyl)glycinate) (11.5 g, 54.4 mmol, 50%) as a colorless oil. LCMS: (ESI, m/z): [M+H]⁺=212.3, ¹H NMR (400 MHz, DMSO-d₆) δ 7.19 (t, J=8.0 Hz, 1H), 7.07 (d, J=11.2 Hz, 1H), 7.02 (d, J=8.0 Hz, 1H), 3.69-3.67 (m, 2H), 3.62 (s, 3H), 3.30-3.27 (m, 2H), 2.20 (s, 3H).

To a solution of F-2 (methyl (3-fluoro-4-methylbenzyl)glycinate) (11.5 g, 54.4 mmol, 1.0 eq) and EtOH (120 mL) was added di-tert-butyl dicarbonate (1.1 g, 5.0 mmol, 1.1 eq). The reaction mixture was stirred at 25° C. for 2 h, then concentrated under reduced pressure to provide F-3 (17.0 g, 54.6 mmol, 100%) as a white solid, which was used without further purification. LCMS: (ESI, m/z): [M+H−Boc]⁺=212.3.

To a solution of F-3 (17 g, 55 mmol, 1.0 eq) in THF (100 mL)at 0° C. was slowly added sodium hydroxide (4.4 g, 2.0 Eq, 0.11 mol) in water (100 mL). The reaction mixture was stirred at 25° C. for 1 h. The mixture was acidified to pH=5 by addition of a HCl (aq. 1N), diluted with water (200 mL), and extracted with EtOAc (3×300 mL). The combined organic layers were washed with brine (300 mL), dried over anhydrous Na₂SO₄, filtered and concentrated under reduced pressure to provide F-4 (15.6 g, 52.5 mmol, 96%) as a colorless oil. LCMS: (ESI, m/z): [M+H−Boc]⁺=198.1.

Into a 500-mL round bottom flask were added F-4 (N-(tert-butoxycarbonyl)-N-(3-fluoro-4-methylbenzyl)glycine (13.2 g, 44.4 mmol, 1.05 eq), 2-(3l-[1,2,3]triazolo[4,5-b]pyridin-3-yl)-1,1,3,3-tetramethylisouronium hexafluorophosphate(V) (16.8 g, 44.2 mmol, 1.05 eq), N-ethyl-N-isopropylpropan-2-amine(16.4 g, 127 mmol, 3.0 eq), and DMF (200 mL). The reaction mixture was stirred at 20° C. for 10 minutes, then 4-((((9H-fluoren-9-yl)methoxy)carbonyl)amino)piperidine-4-carboxylic acid hydrochloride (17.0 g, 1.00 Eq, 42.2 mmol) was added. The reaction mixture was stirred at 25° C. for an additional 2 h and concentrated. The residue was purified by MPLC and the collected fractions were concentrated under reduced pressure and dried by lyophilization to provide F-5 (11.6 g, 18.0 mmol, 42.6%) as a white solid. LCMS: (ESI, m/z): [M+H]+=646.3. ¹H NMR (300 MHz, DMSO-d₆) δ 7.89 (d, =J 7.5 Hz, 2H), 7.73 (d, J=7.4 Hz, 2H), 7.67 (s, 1H), 7.42 (td, J=74, 1.2 Hz, 2H), 7.33 (t, J=7.4 Hz, 2H), 7.23 (t, J=8.0 Hz, 1H), 7.08-6.95 (m, 2H), 4.45-4.17 (m, 5H), 4.05-3.90 (m, 3H), 3.60-3.50(m, 1H), 3.20-3.10 (m, 1H), 3.03-2.90 (m, 1H), 2.21 (s, 3H), 2.05-1.95 (m, 2H), 1.88-1.65 (m, 2H), 1.38-1.32 (m, 9H).

### Procedure G: (S)-6˜((5-((((9H-fluoren-9-yl)methoxy)carbonyl)amino)-6-(tert-butoxy)-6-oxohexyl)amino)-6-oxohexanoic acid

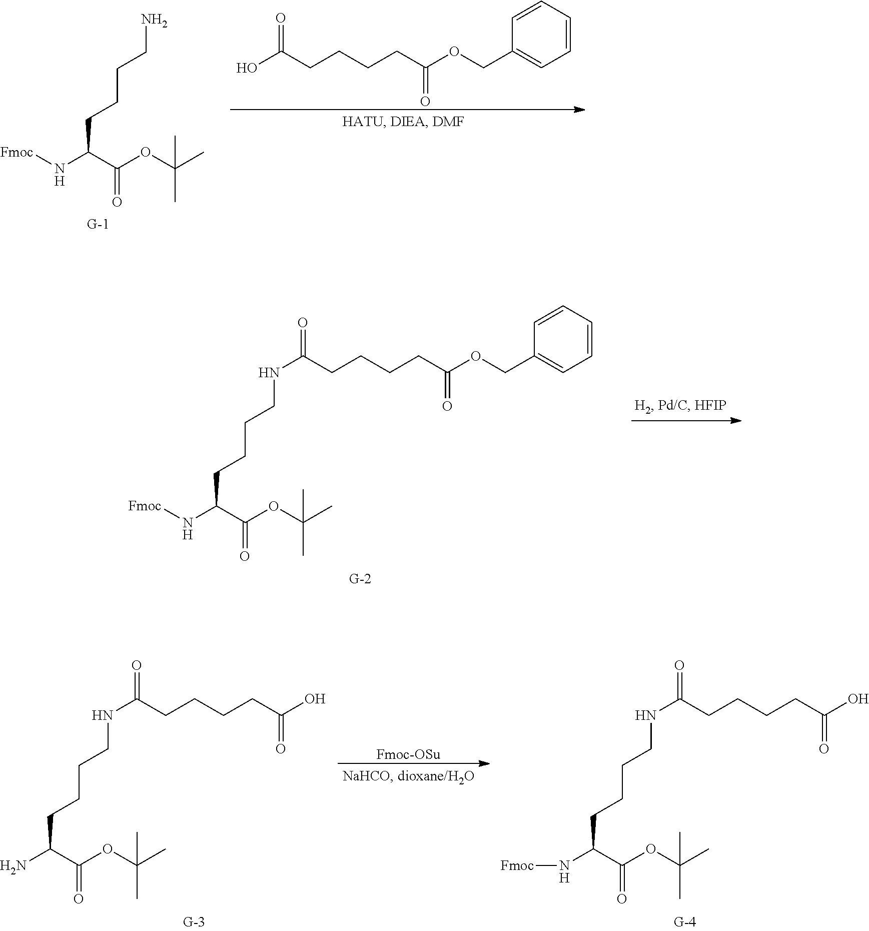

To a solution of 6-(benzyloxy)-6-oxohexanoic acid (5.0 g, 2116 mmol, 1.5 Eq) and G-1(6.50 g, 14.11 mmol, 1.0 Eq) in DMF (50 mL) were added HATU (8.05 g, 21.16 mmol, 1.5 eq) and DIEA (7.29 g, 56.43 mmol, 4.0 eq). The mixture was stirred for 1 h at 25° C. and extracted with EtOAc (3×500 mL). The combined organic layers were washed with brine (3×500 mL), then dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 90% gradient in 30 min; detector, UV 254 nm to afford G-2 (8.1 g, 83.6%) as a white solid. LCMS: (ESI, m/z): [M+H]643.40. ¹H NMR (400 MHz, DMSO-d₆) δ 7.90 (d, J=7.5 Hz, 2H), 7.78 (t, J=5.6 Hz, 1H), 7.73 (d, J=7.5 Hz 1H), 7.65 (d, J=7.8 Hz, 1H), 7.46-7.39 (M, 2H), 7.37-7.31 (m, 6H), 5.08 (s, 2H), 4.36-4.19 (m, 3H), 3.90-3.80 (m, 1H), 3.09-2.95 (m, 2H), 2.39-2.31 (n, 2H), 2.08 (s, 4H), 1.69-1.56 (m, 2H), 1.51 (p, J=2.6 Hz, 4H), 1.39 (s, 1H).

To a solution of G-2 (3.0 g, 4.67 mmol, 1.0 Eq)in 1,1,1,3,3,3-hexafluoroisopropanol (30 mL) was added Pd/C (0.1 g, 0.94 mmol, 20 wt. %)in a 100 mL round-bottom flask. The mixture was hydrogenated at room temperature overnight under hydrogen atmosphere using a hydrogen balloon, filtered through a celite pad and concentrated under reduced pressure to afford G-3 (2.9 g, crude) as a white solid. The product was used in the next step without further purification. LCMS: (ESI, m/z): [M+H⁺]=331.30.

To a mixture of G-3 (2.9 g, 9.08 mmol, 1.0 Eq) in 1,4-dioxane (225 mL) and water (75 mL) was added solid NaHCO₃. After the pH of the solution was adjusted to 8˜9, Fmoc-OSu (1.84 g, 5.45 mmol, 0.60 Eq) was added in portions at room temperature. The resulting mixture was stirred for 16 h at room temperature and extracted with EtOAc (3×750 mL). The combined organic layers were washed with water (3×700 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 40% to 70% gradient in 15 min; detector, UV 254 nm to afford G-4 (4.5 g, 73.6%) as a light brown oil. LCMS: (ESI, m/z): [M+H⁺]=553.35. ¹H NMR (400 MHz, DMSO-d₆) δ 12.00 (s, 1H), 7.98-7.83 (m, 2H), 7.73 (td, J=13.7, 6.9 Hz, 3H), 7.64 (d, J=7.8 Hz, 1H), 7.49-7.36 (m, 21H), 7.33 (td, J 7.74, 1.2 Hz, 2H), 4.27 (t, J=14.0, 7.2 Hz, 3H), 3.01 (q, J=6.6 Hz, 2H), 2.19 (t, J=6.7 Hz, 2H), 2.08 (s, 4H), 1.60 (dq, J=22.8, 8.4, 7.7 Hz, 2H), 1.47 (dq, J=6.9, 4.0 Hz, 4H), 1.39 (m, 1H).

### Procedure H: Synthesis of Compound 1 (1-3 Disclosed as SEQ ID NO: 824 And Compound 1 Disclosed as SEQ ID NO: 391)

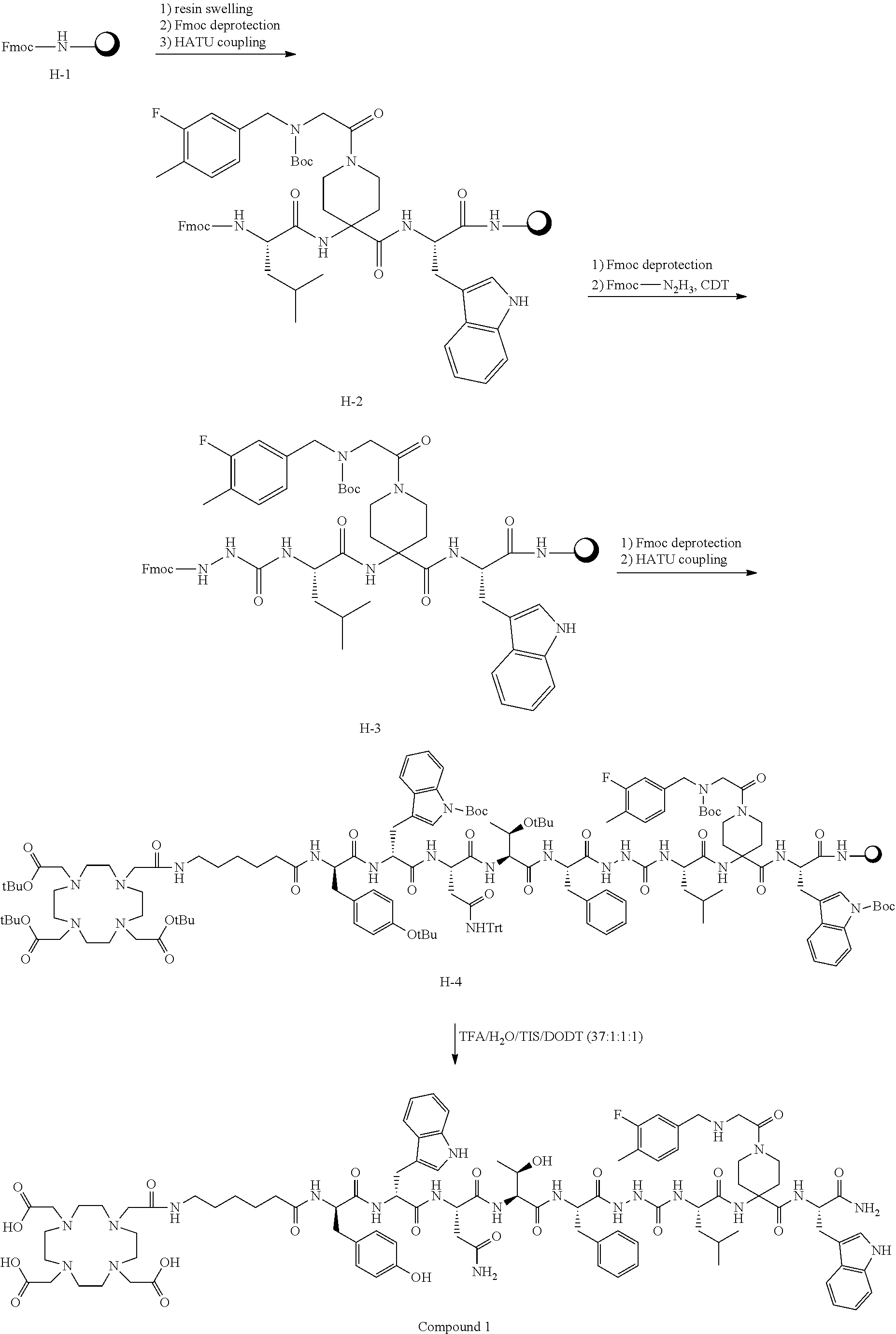

Peptide H-2 was prepared by standard Fmoc-based SPPS using N^{α}-Fmoc-Rink amide resin. The details were outlined in Procedure A above. The coupling reaction with precursor F-5 was performed twice with HATU at 60° C.

Peptide H-2 was treated with the mixture of Fmoc-N₂H₃ (3.0 Eq) and CDT (3.0 Eq) in NMP (10 mL/g resin) overnight, at room temperature, and under nitrogen atmosphere. The reaction was operated manually. The reaction was washed with NMP (3×2 mL) to afford peptide H-3 on resin.

Elongation of H-3 was carried out by repeating Procedures A-3 and A-4 until peptide H-4 was obtained.

Peptide H-4 was cleaved from the resin, followed by purification by RP-HPLC according to Procedures A-7. The desired fractions were concentrated and lyophilized to afford Compound 1 as a white solid (overall yield 8.54%, 98.5% purity). MS Calc'd for C₉₃H₁₂₄FN₂₁O₂₁: 1889.9, found [M+2H]²⁺: 946.4.

### Procedure I: Synthesis of Compound 332 (1-3 Disclosed as SEQ ID NO: 825, 1-4 Disclosed as SEQ ID NO: 826, 1-5 Disclosed as SEQ ID NO: 827 and Compound 332 Disclosed as SEQ ID NO: 714)

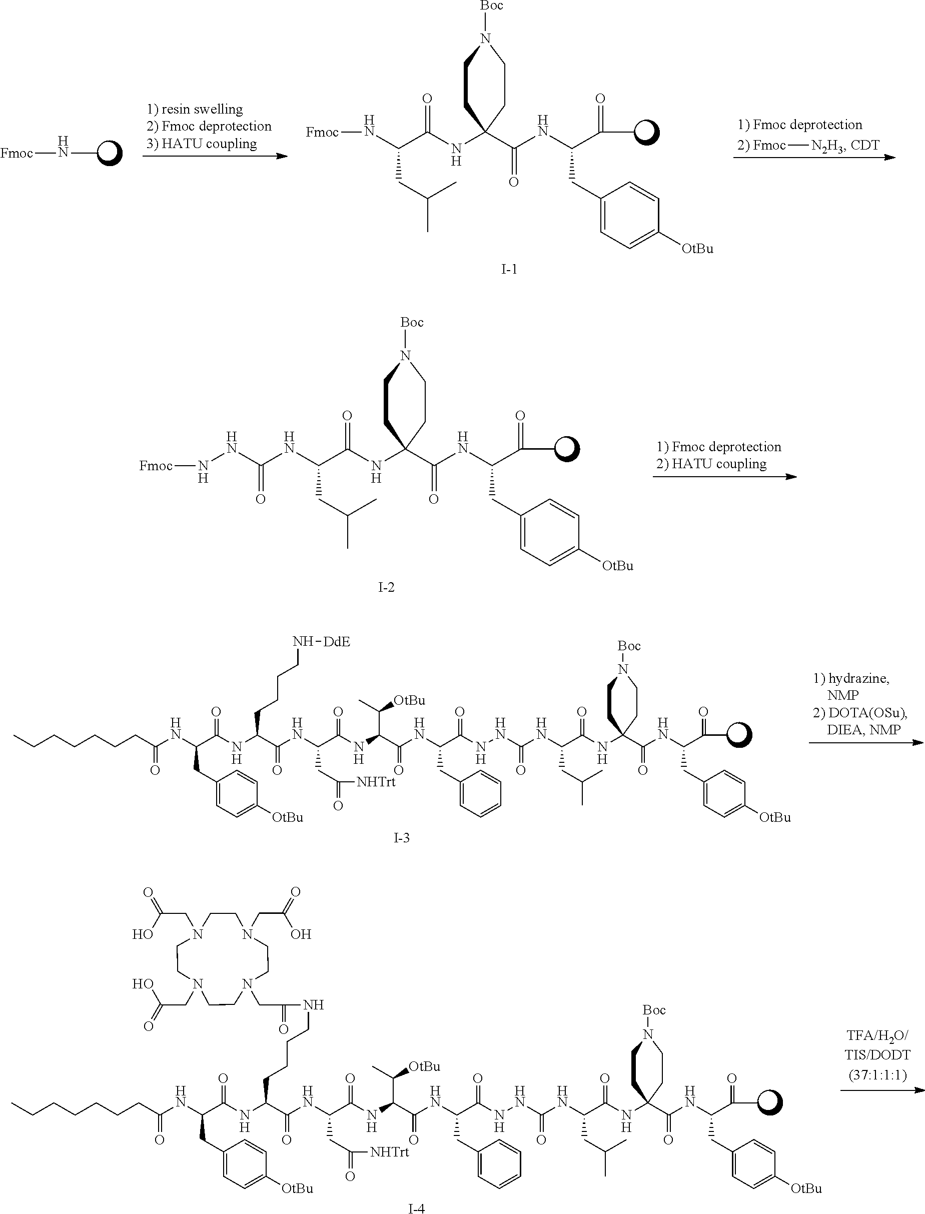

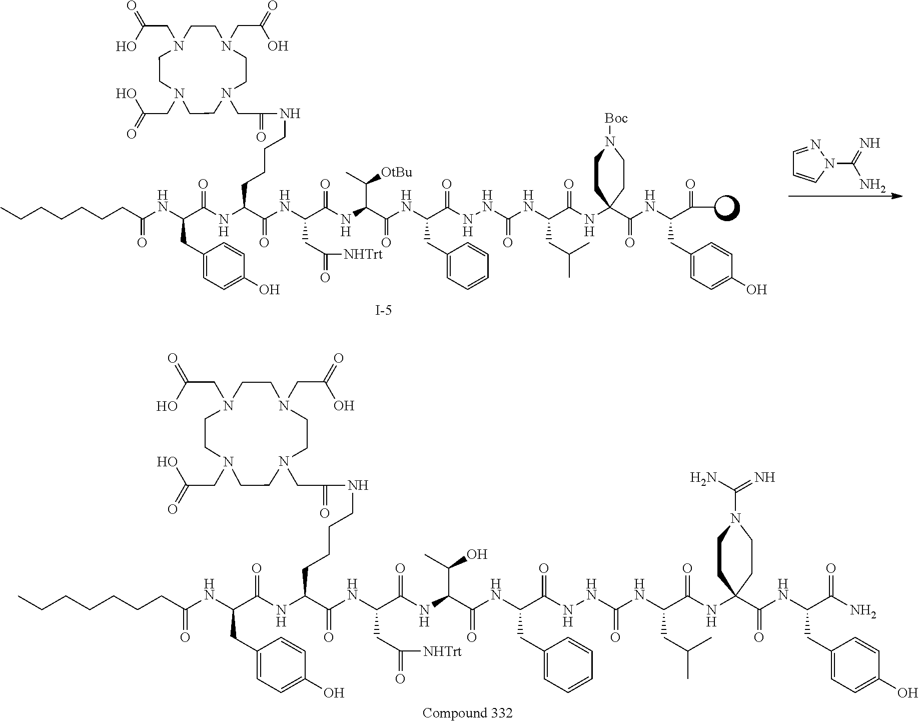

Peptide 1-1 was prepared by standard Fmoc-based SPPS using N^{α}-Fmoc-Rink amide resin. The details were outlined in Procedure A above (A-2, A-3, and A-4).

Peptide I-1 was treated with the mixture of Fmoc-N₂H₃ (3.0 Eq) and CDT (3.0 Eq) in NMP (10 mL/g resin) overnight at room temperature under nitrogen atmosphere. The reaction was operated manually. The reaction was washed with NMP (3×2 mL) to afford peptide 1-2 on resin.

Elongation of I-2 was carried out by repeating Procedures A-3 and A-4 until peptide I-3 was obtained.

1-3 on resin (0.45 g) was treated with 2% hydrazine hydrate in NMP (5 mL) for 2 h under nitrogen atmosphere and then washed with NP (6×15 mL). To the resin in NMP (15 mL) were added DOTA(OSu) (279.8 mg, 0.56 mmol, 2.0 Eq) and DIEA (144.0 mg, 1.12 mmol, 4.0 Eq). The mixture was incubated for 2 h at room temperature and washed with NMP (6×15 mL) to afford 1-4. The crude peptide was cleaved from the resin with a solution of TFA/H₂O/TIS/DODT (37:1:1:1 v/v/v/v, 15 mL) for 2 h at room temperature. The mixture was filtered, and the filtrate was concentrated under reduced pressure. The crude product was purified by RP-HPLC. The desired fractions were combined, concentrated and lyophilized to afford peptide 1-5 (90.0 mg, overall yield 19.6%) as a white solid.

To a solution of peptide I-5 (90.0 mg, 0.055 mmol, 1.0 Eq) in DMF (1.0 mL) were added 1H-pyrazole-1-carboxamidine (60.3 mg, 0.550 mmol, 100 Eq) and DIEA (708 ng, 0.550 mmol, 10.0 Eq). The resulting mixture was stirred for 4 h at 30° C. The crude product was purified by RP-HPLC. The desired fractions were combined, concentrated and lyophilized to afford Compound 332 (69.2 mg, 98.9% purity, 74.2% yield) as a white solid. MS Calc'd 1684.89, found [M+2H]²⁺:843.95.

### Procedure J: General Synthesis Procedure for Metal Complexation

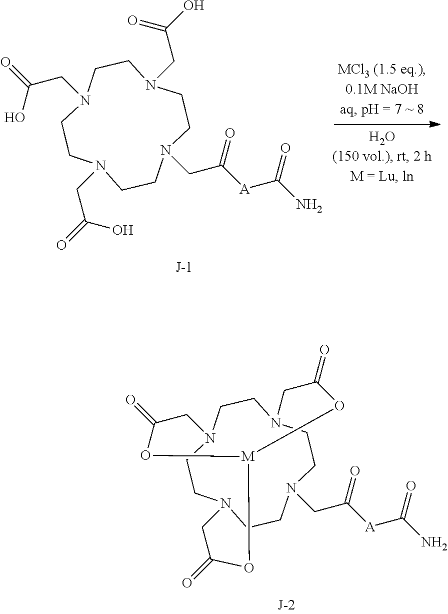

Compound J-1 (1.0 Eq) and MCl₃ (1.5 Eq) were dissolved in H₂O (150.0 vol). The mixture was neutralized with 0.1 M aq. NaOH to pH=7-8. The mixture was stirred for 2 h at room temperature. The reaction was monitored by LCMS. Upon completion, the mixture was centrifuged, and the precipitate was purified by prep-HPLC and dried by lyophilization afford J-2.

In compounds J-1 and J-2, “A” is a generic representation for the portion of each radionuclide conjugate molecule that connects the DOTA group to the amidated C-terminal carboxyl group of the peptide ligand. In other words, “A” is a generic representation for the portion of each radionuclide conjugate molecule that connects R^{a}(or R^{a} complexed with a metal) to the amidated C-terminal carboxyl group of the peptide ligand.

### Procedure K: General Synthesis Procedure for ¹¹¹In-labeling

[¹¹¹In]InCl₃ in HCl was added to a solution of a ligand in NH₄OAc or NaOAc buffer (0.1 M, pH 5.0-5.5). The resulting mixture was heated at 60-95° C. in a thermal mixer for 15-30 min. Radiochemical purity was determined using iTLC or radio-HPLC analytical methods. The typical molar activities used in the studies ranged from 5-7 MBq/nmol to 10-15 MBq/nmol. The radiotracer solution for in vivo studies was formulated by dilution of the reaction mixture with 0.9% saline containing proper excipients based on stability studies.

Examples disclosed herein were prepared according to the procedures outlined above.

### EXAMPLES

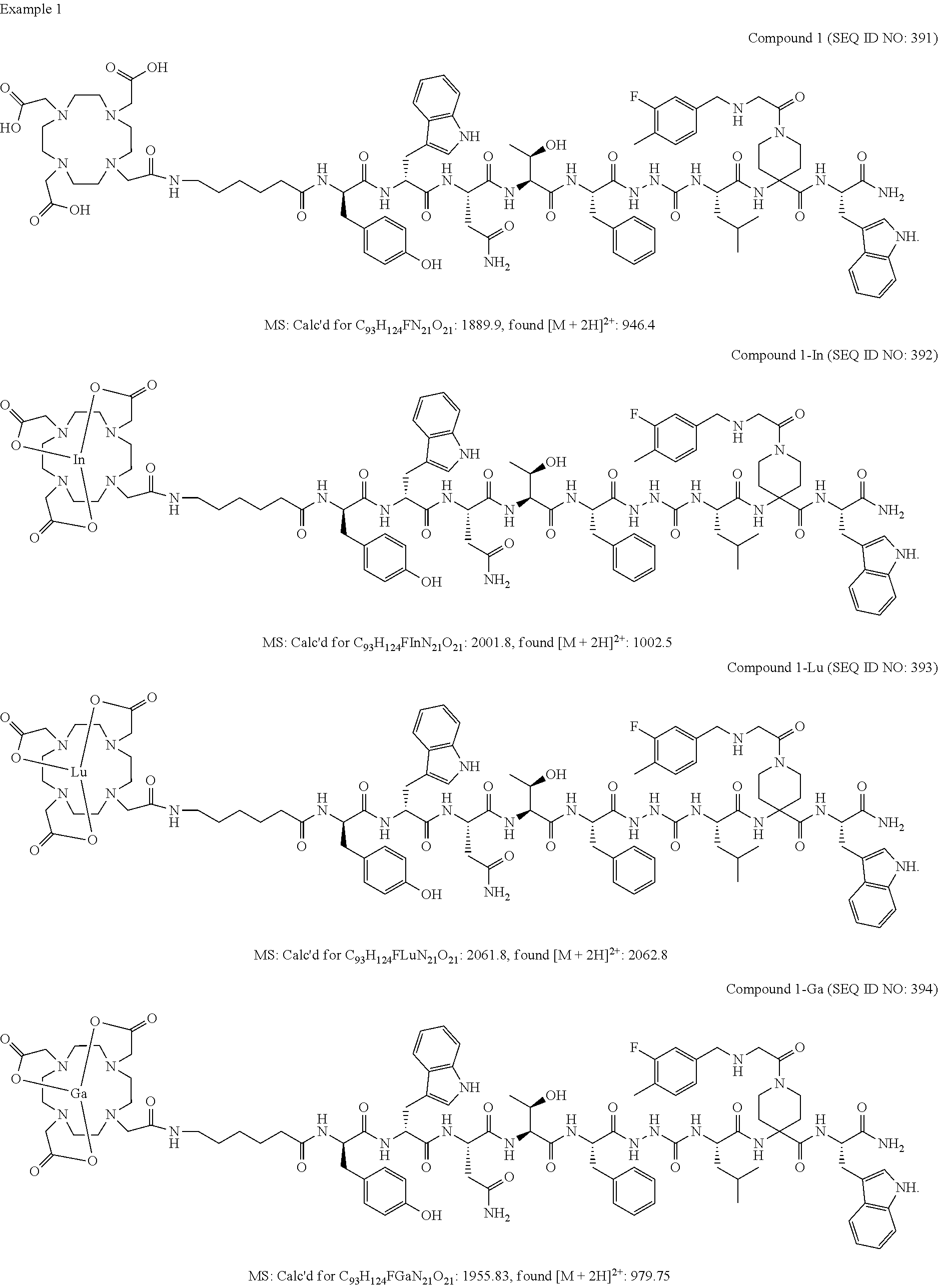

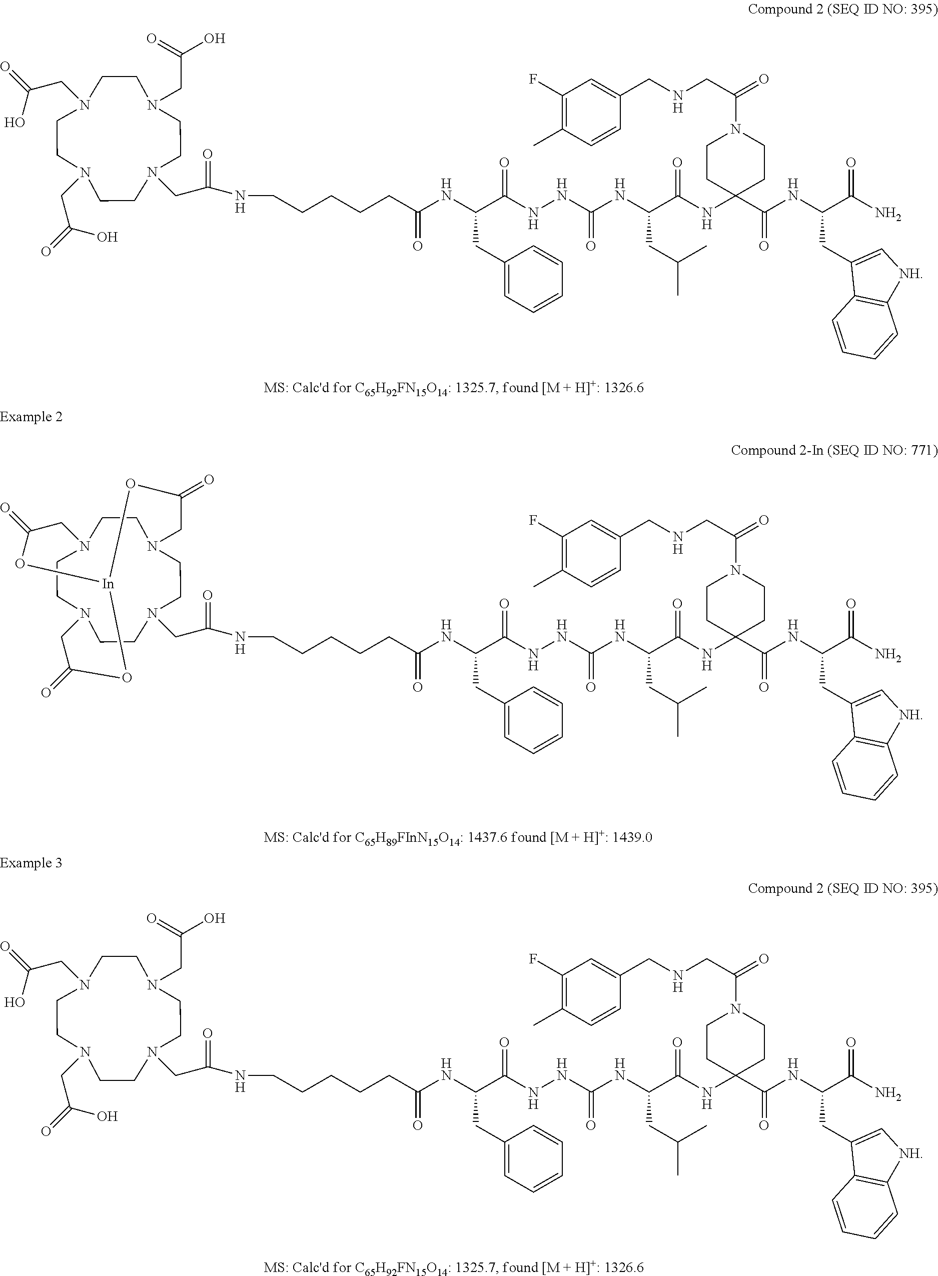

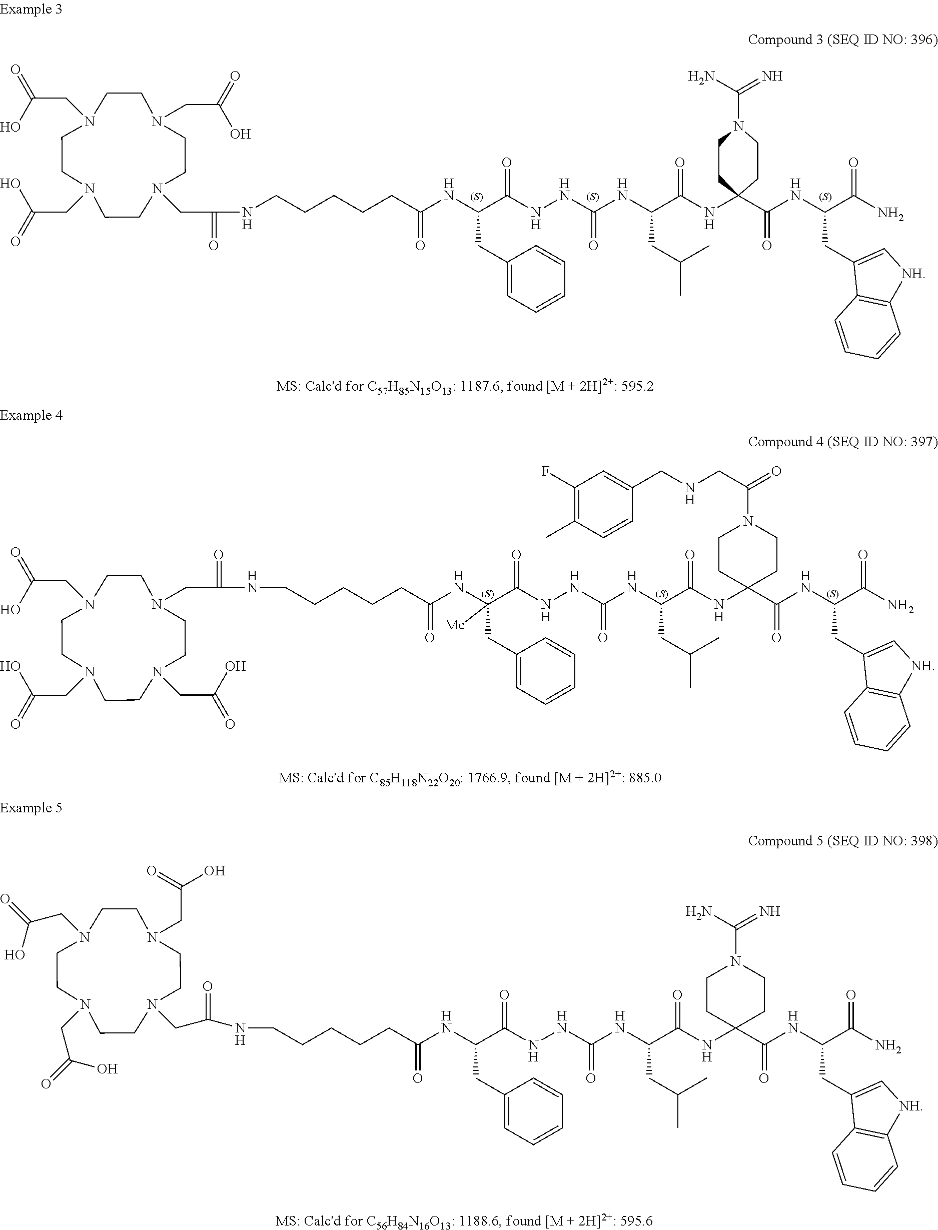

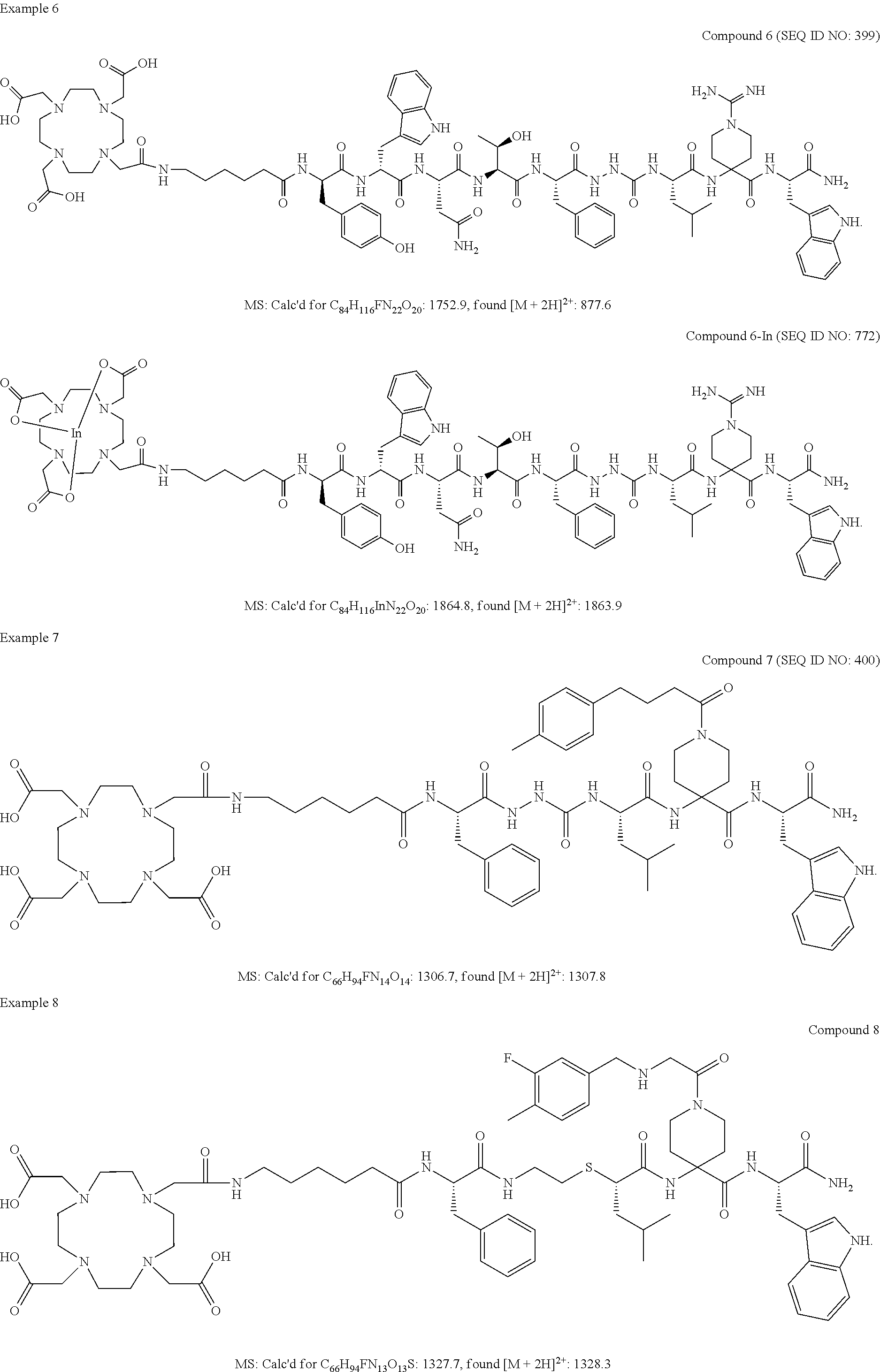

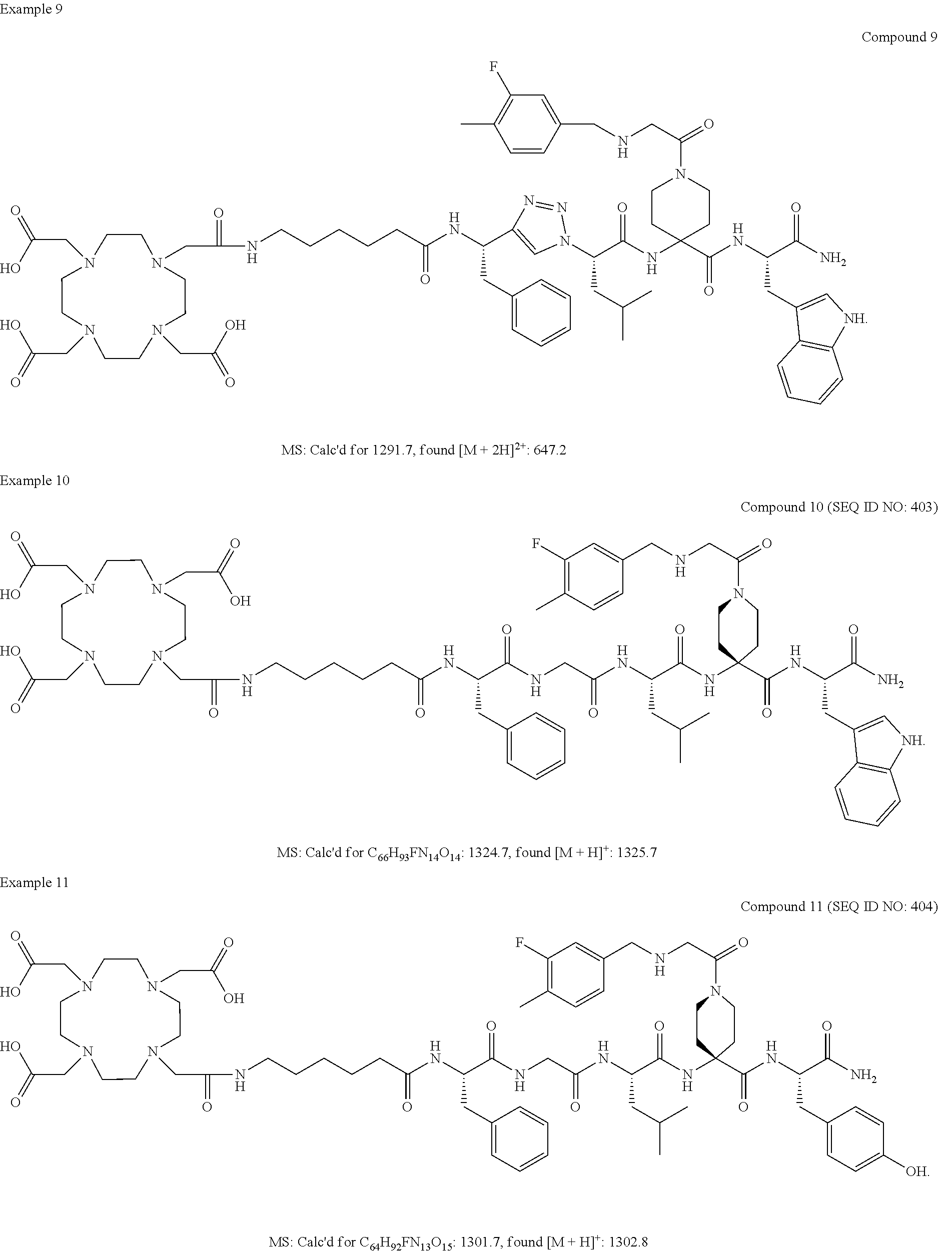

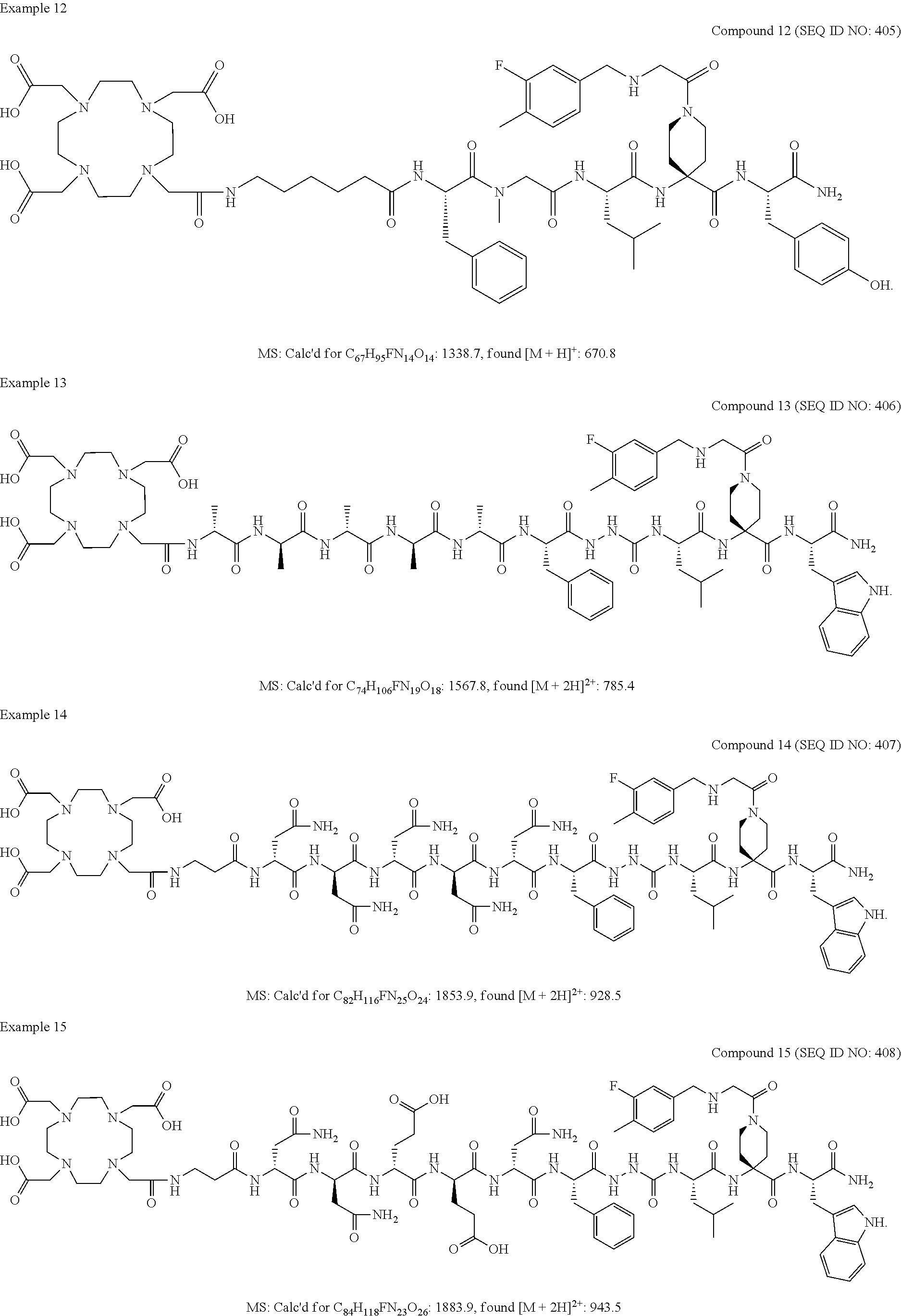

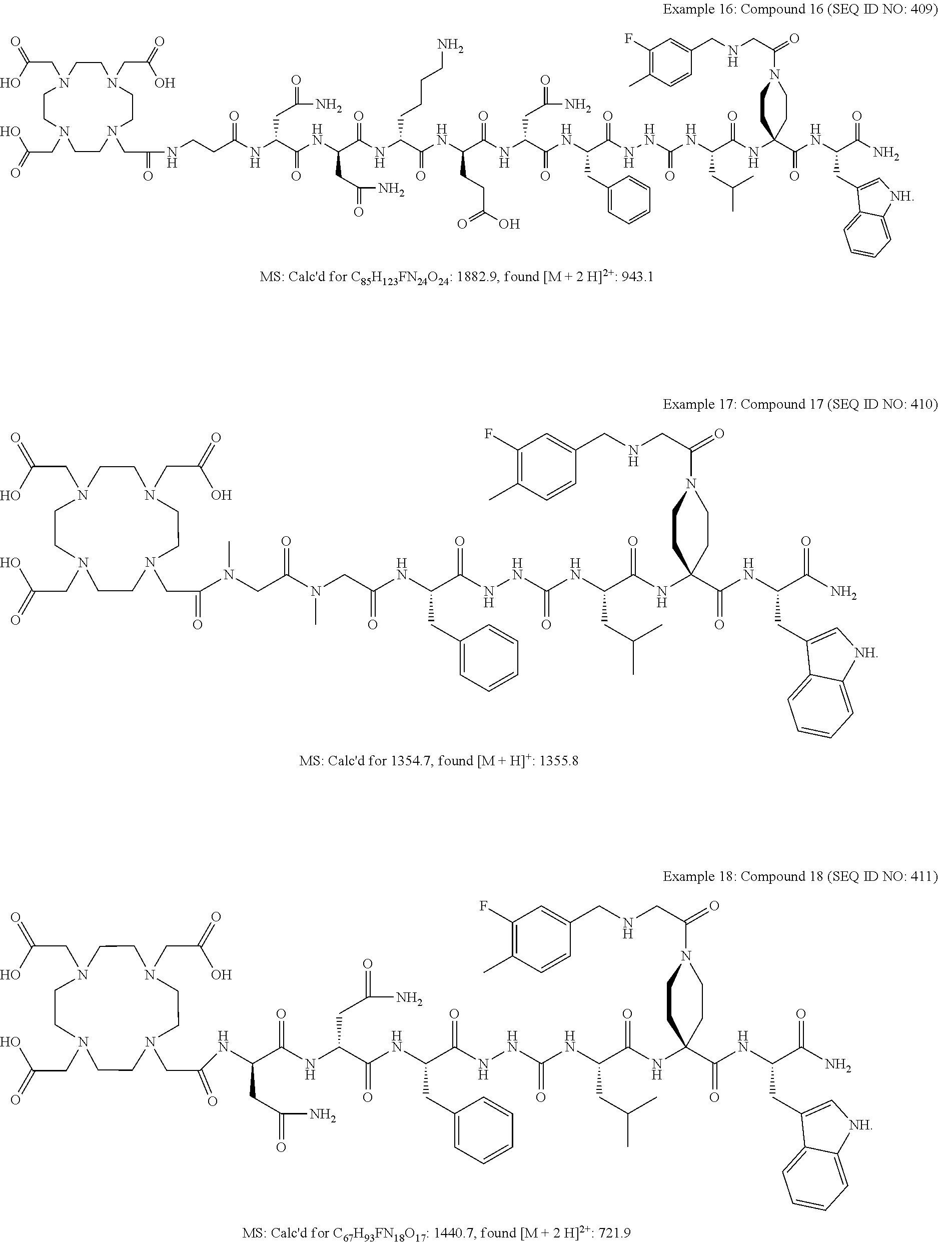

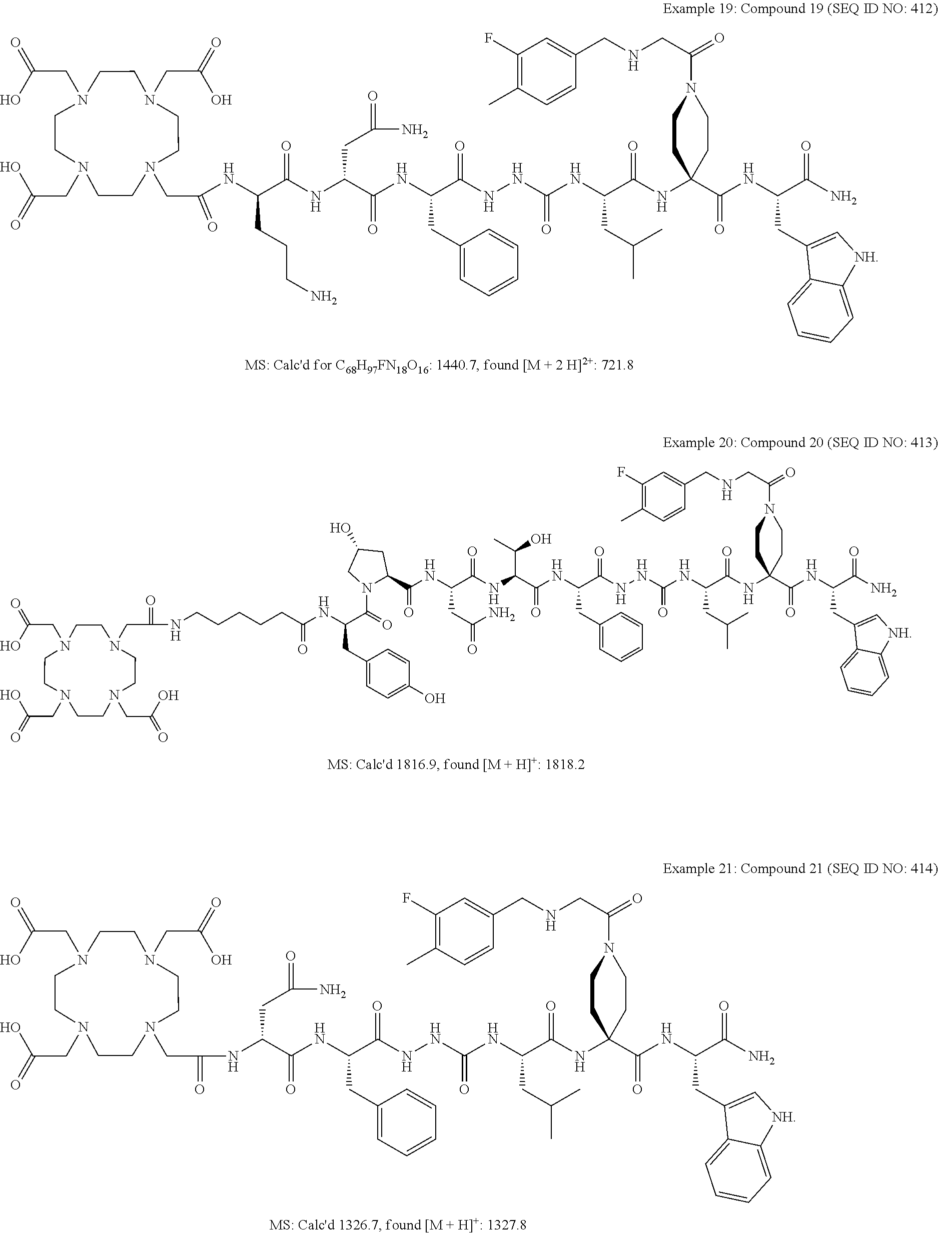

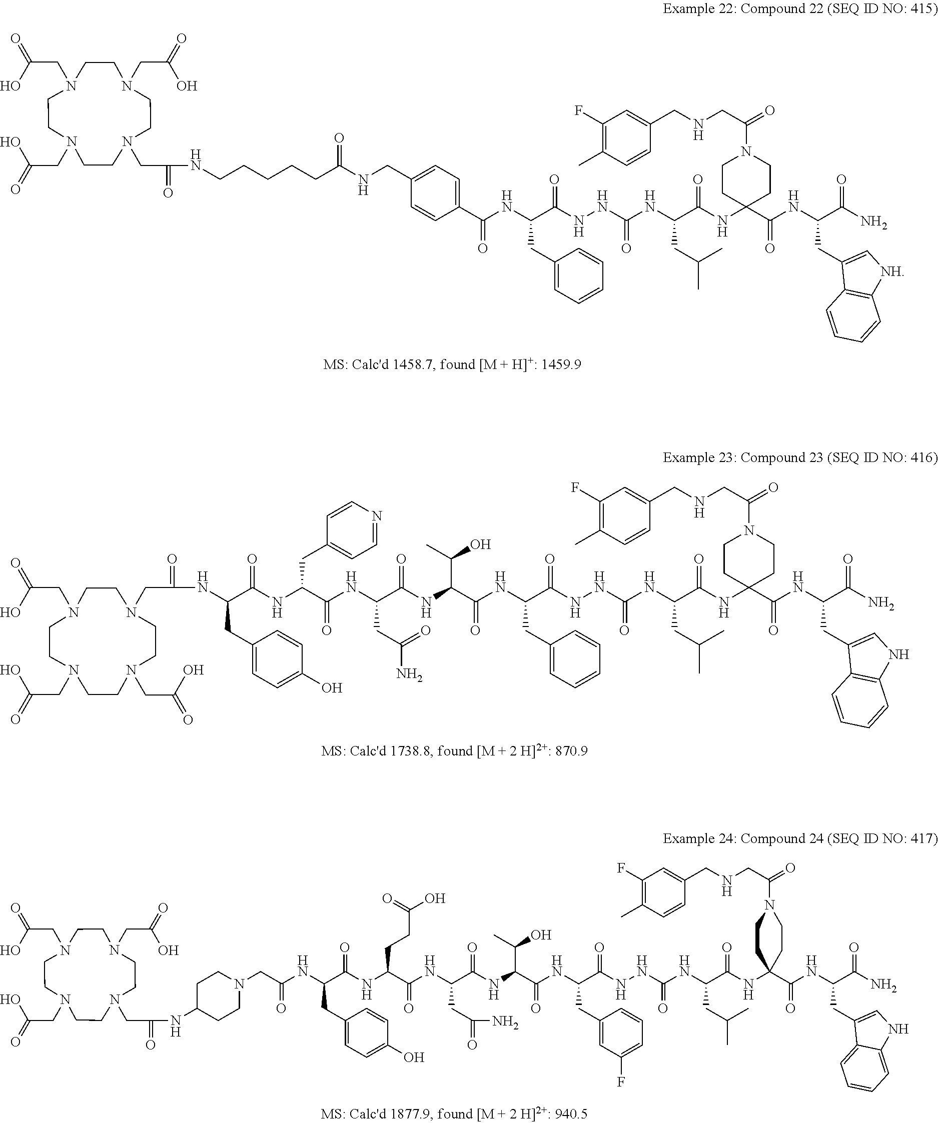

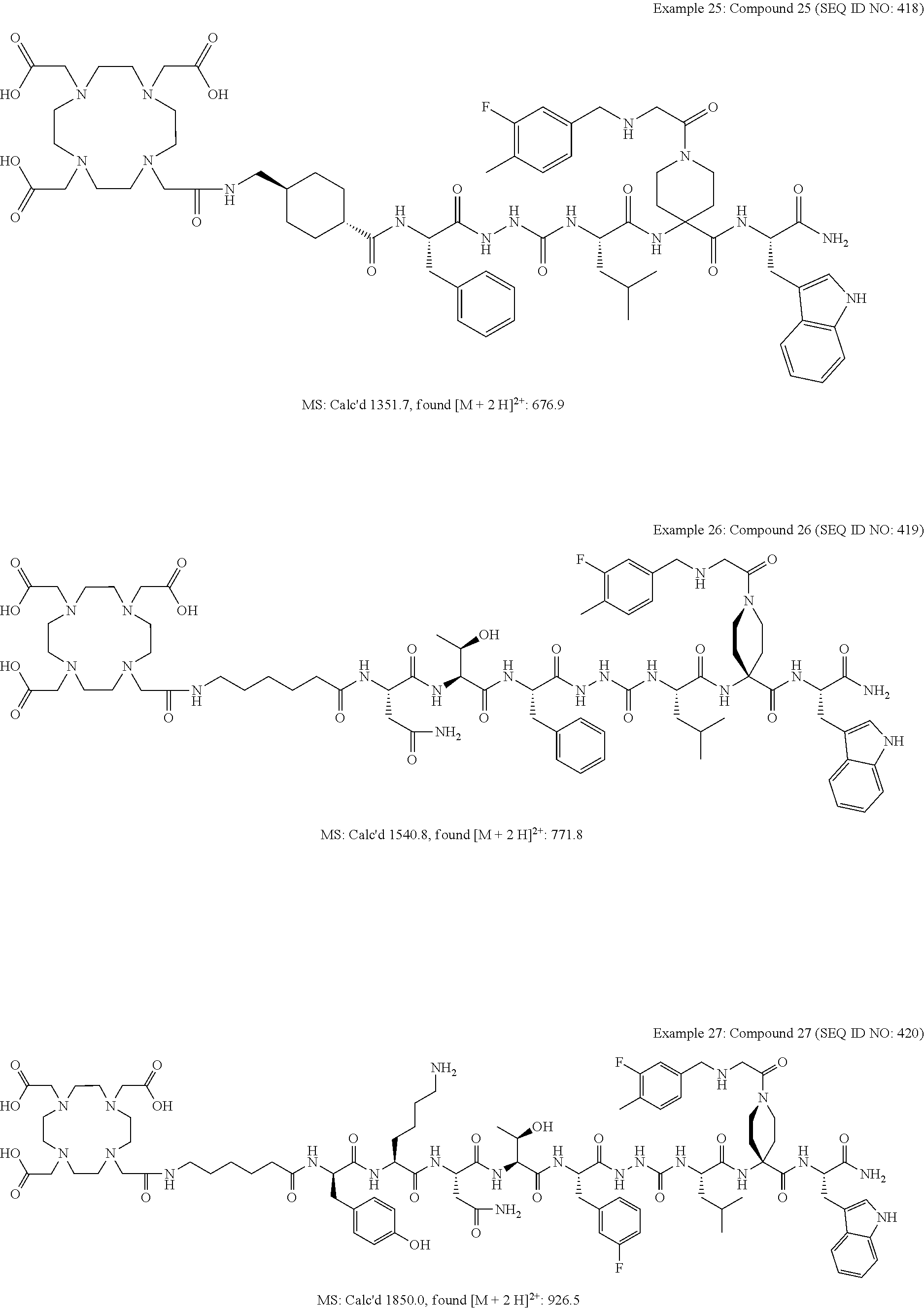

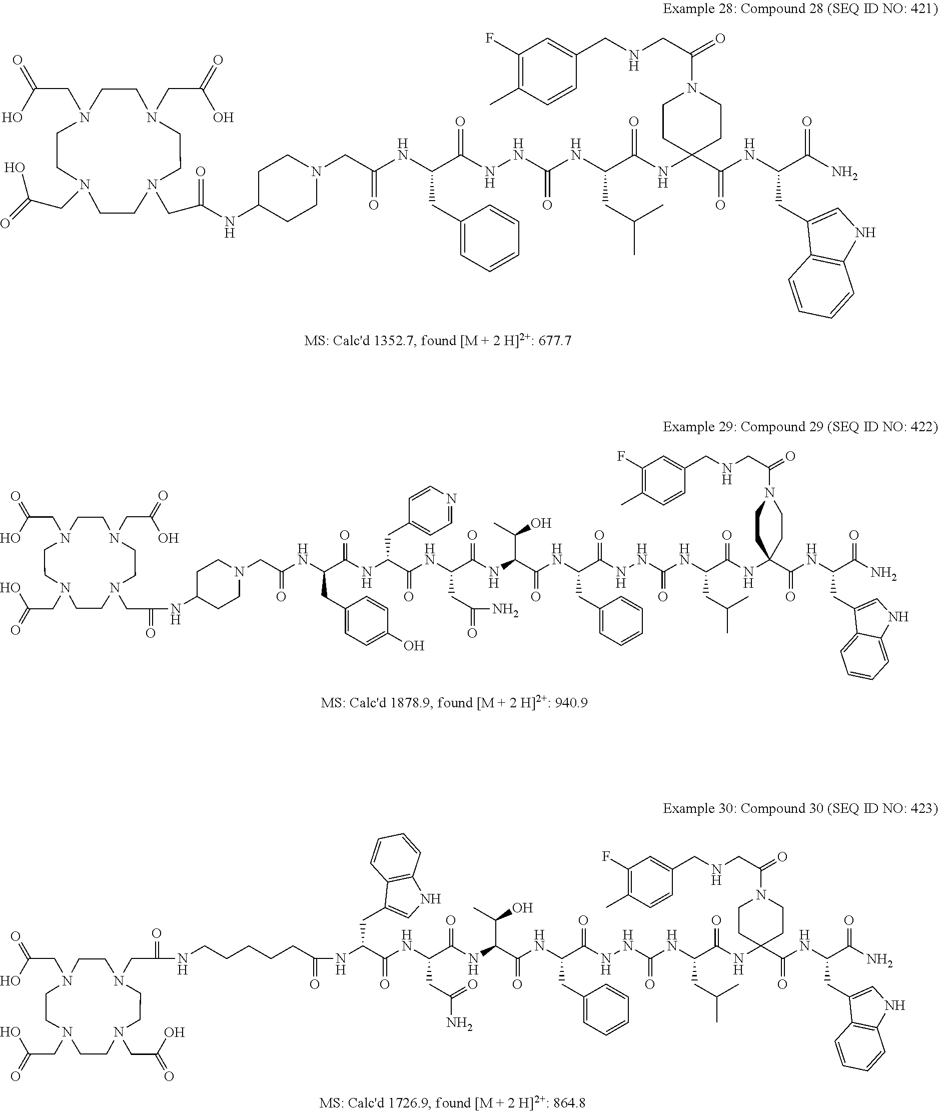

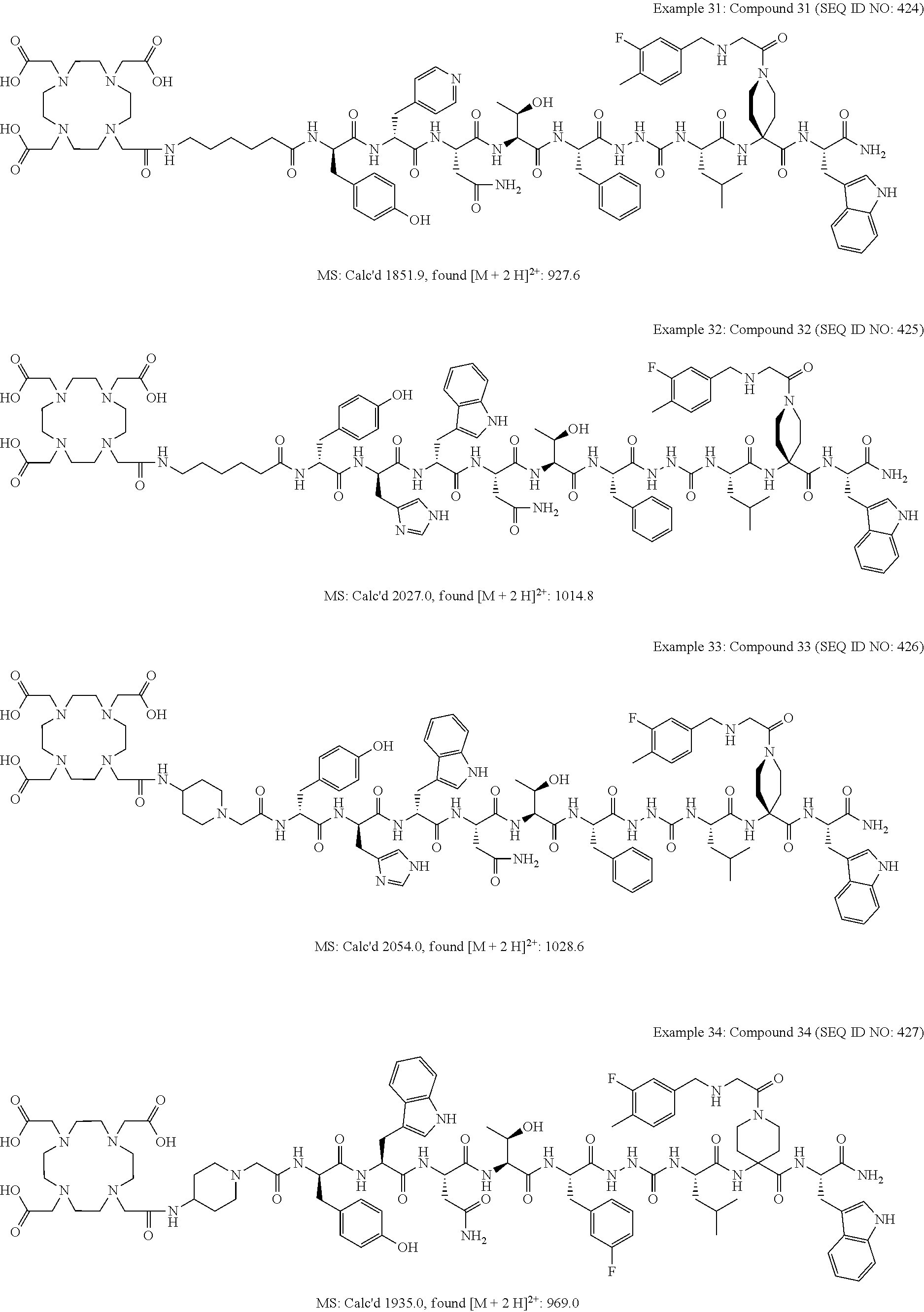

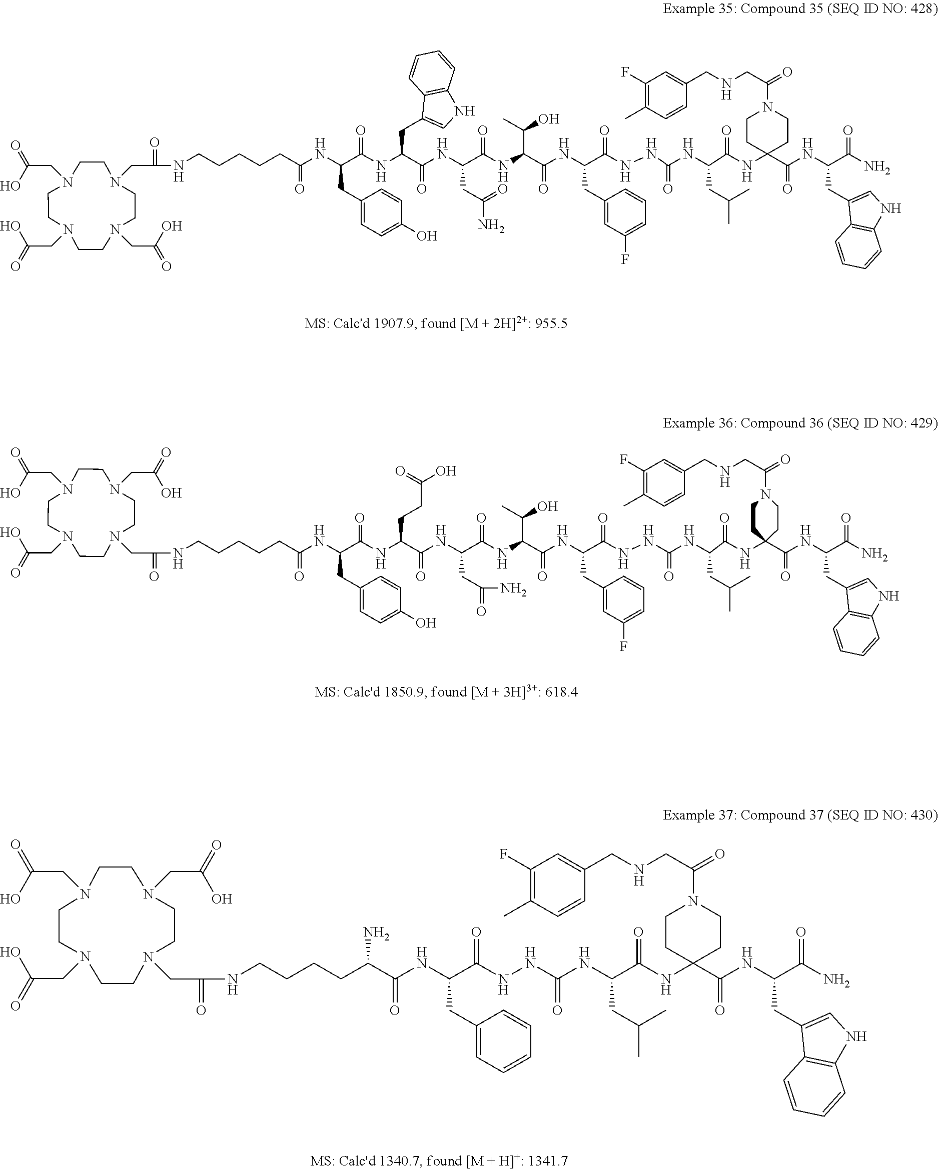

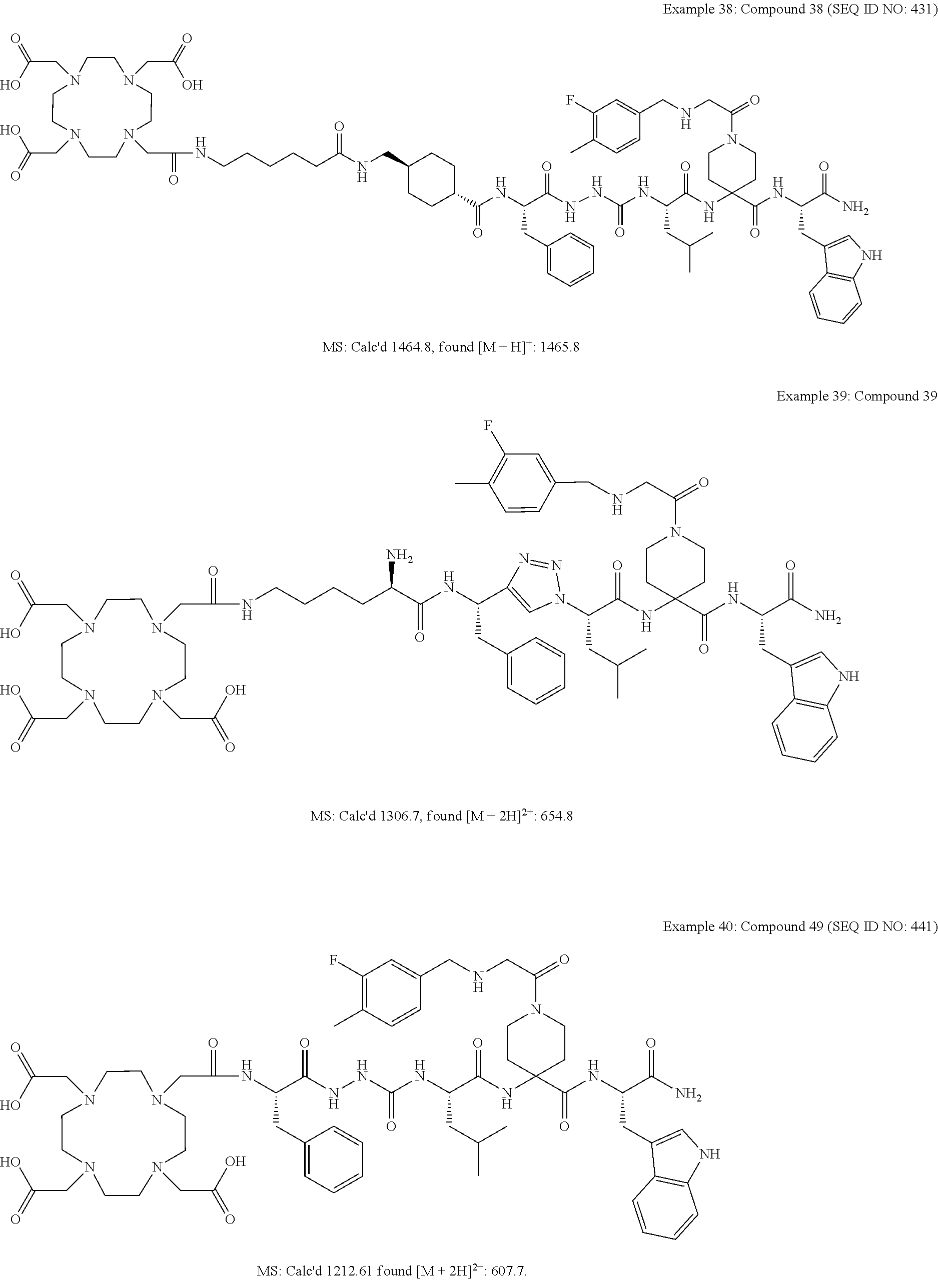

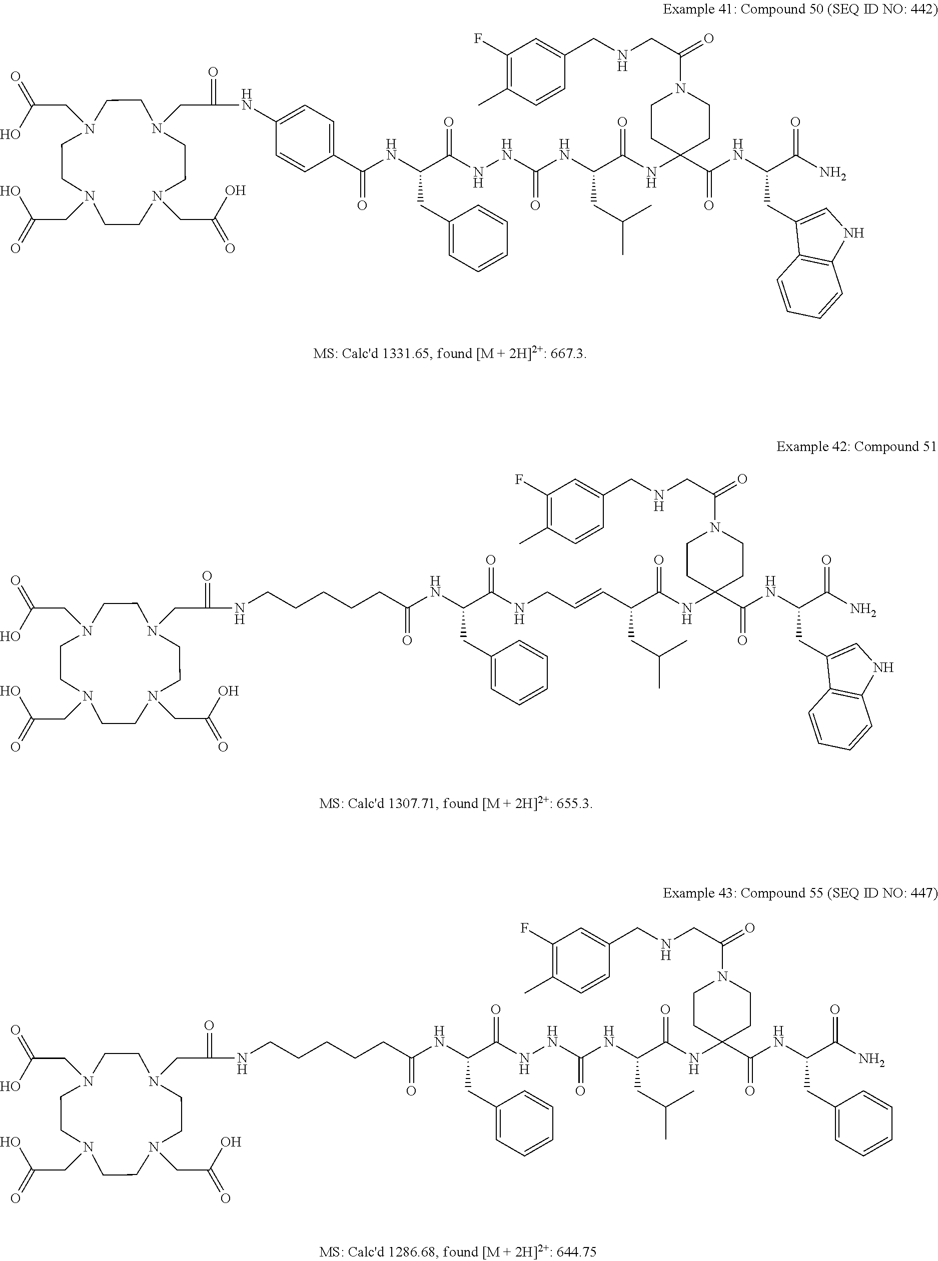

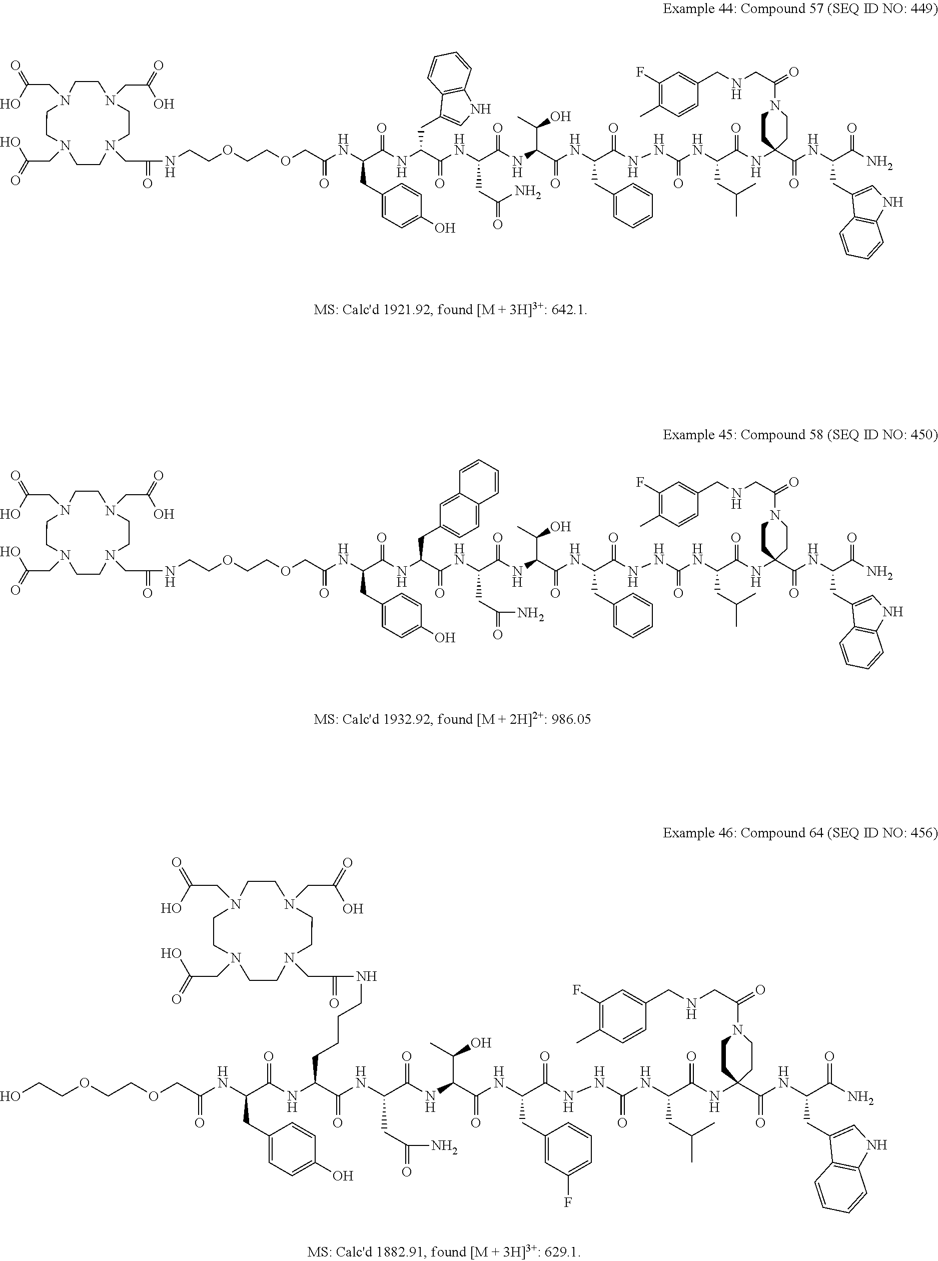

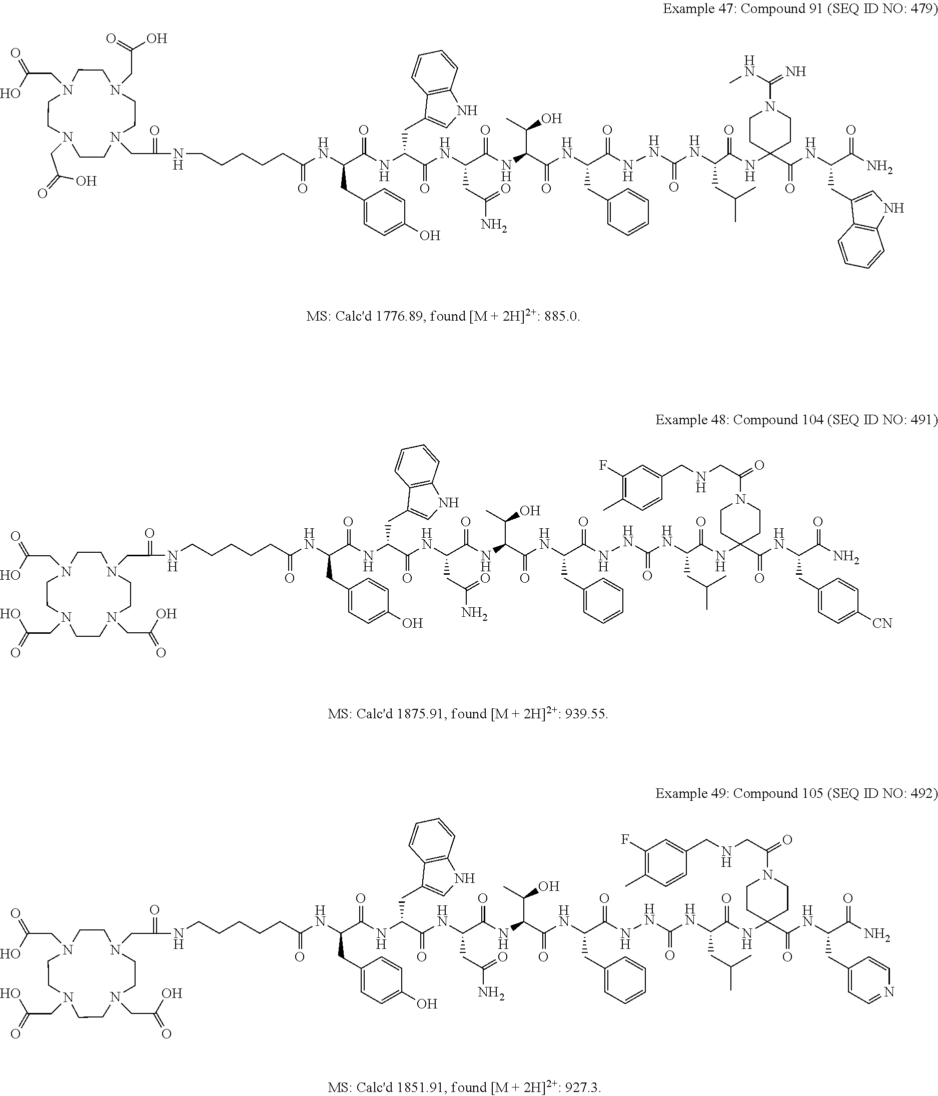

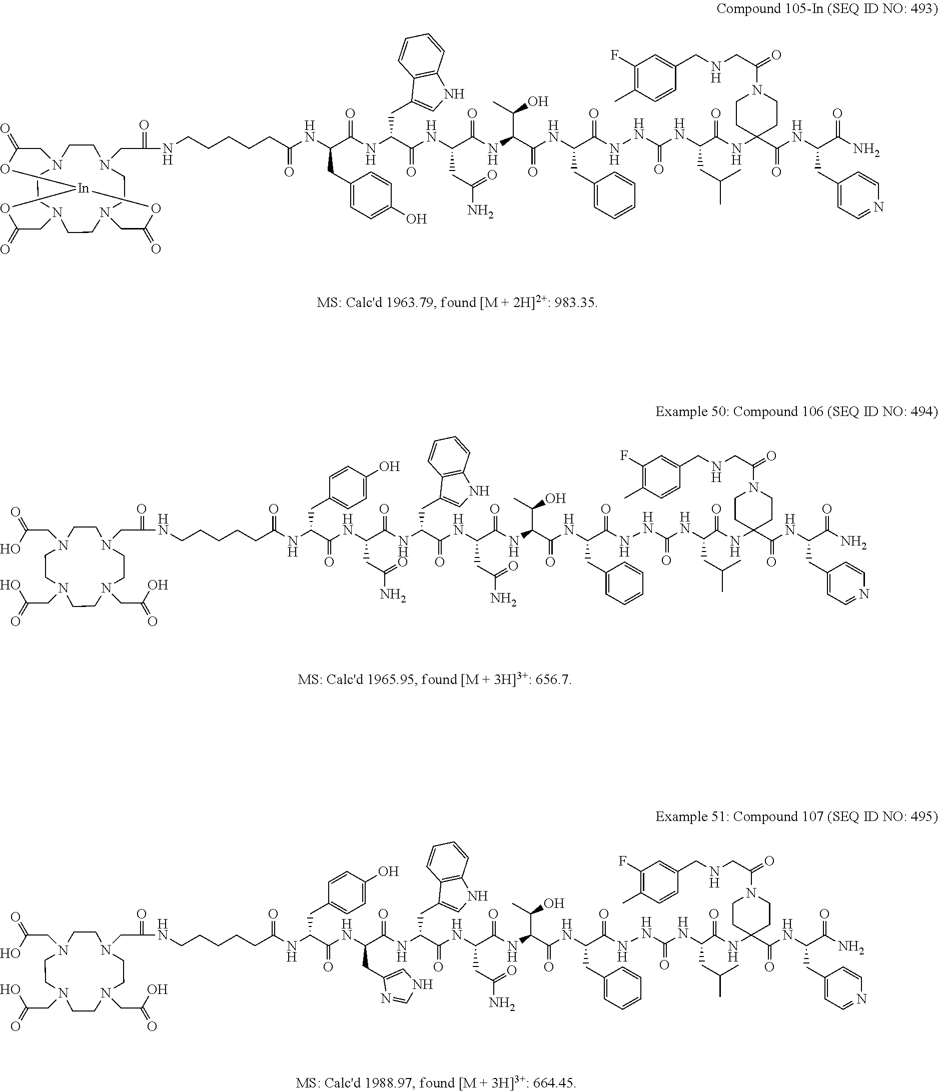

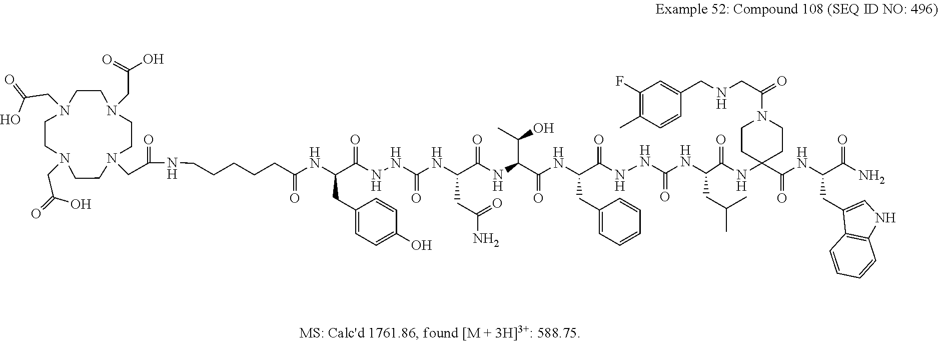 [SMILES: Cc1ccc(CNCC(=O)N2CCC(NC(=O)[C@H](CC(C)C)NC(=O)NNC(=O)[C@H](Cc3ccccc3)NC(=O)[C@@H](NC(=O)[C@H](CC(N)=O)NC(=O)NNC(=O)[C@@H](Cc3ccc(O)cc3)NC(=O)CCCCCNC(=O)CN3CCN(CC(=O)O)CCN(CC(=O)O)CCN(CC(=O)O)CC3)[C@@H](C)O)(C(=O)N[C@@H](Cc3c[nH]c4ccccc34)C(N)=O)CC2)cc1F]

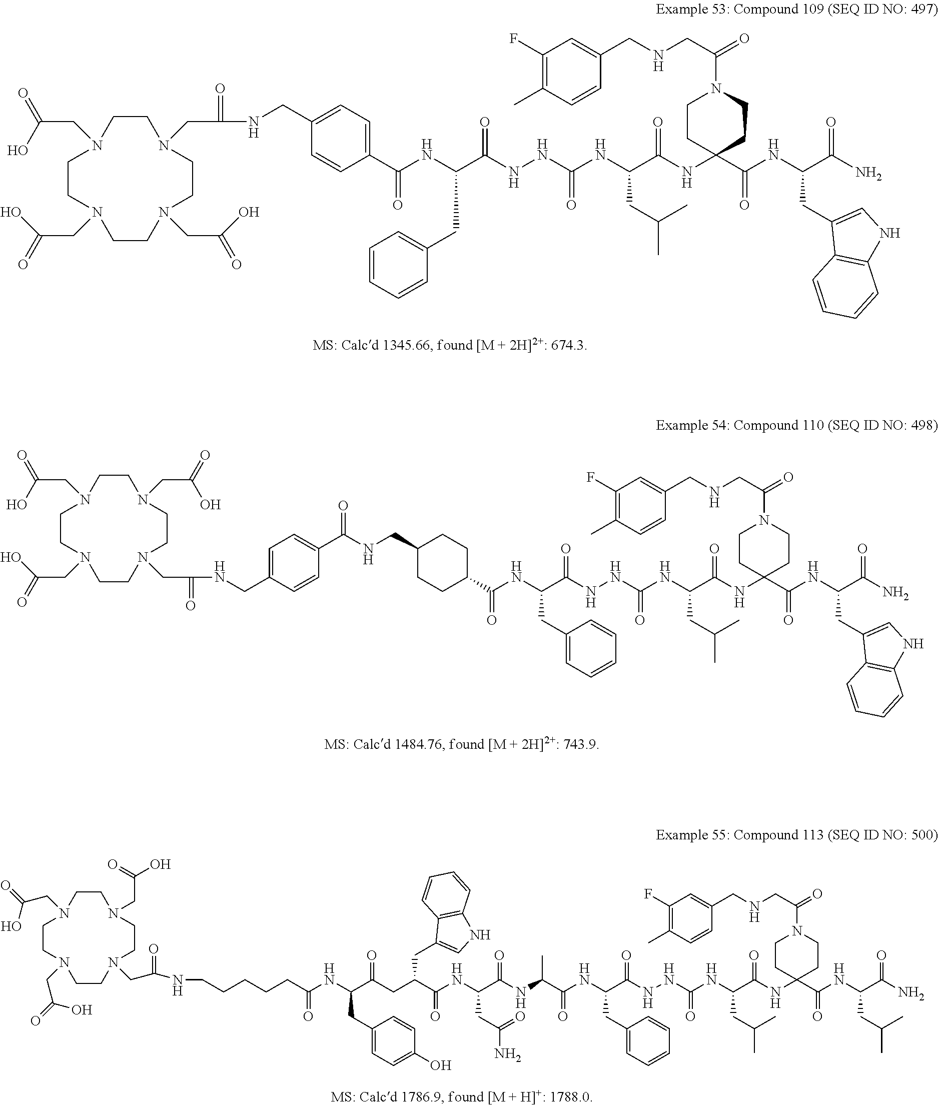

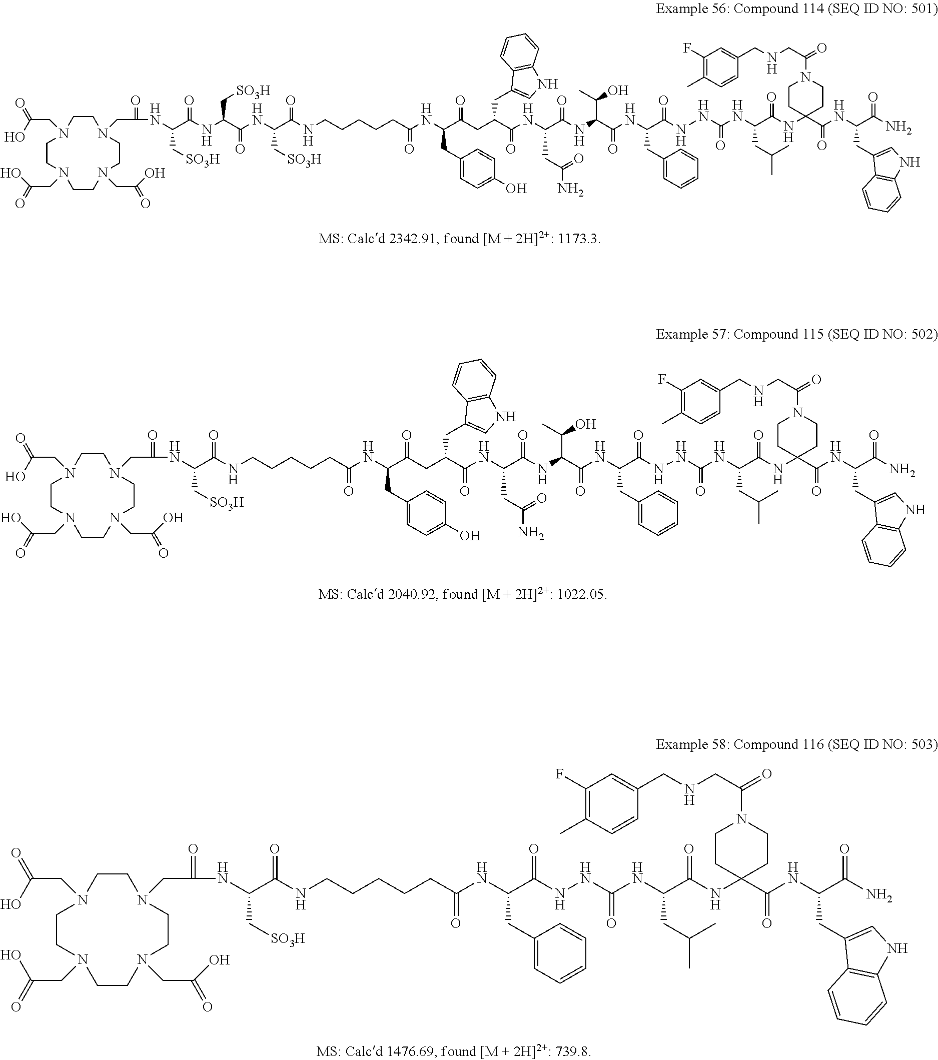

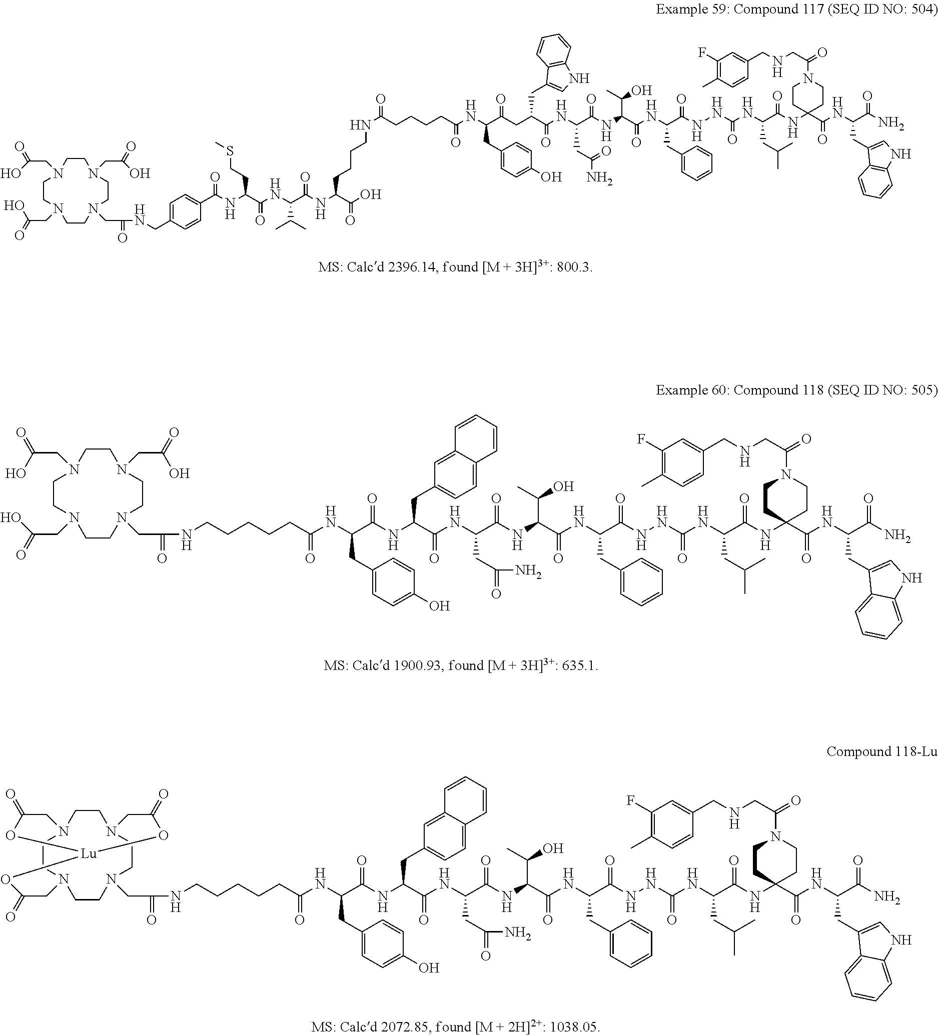

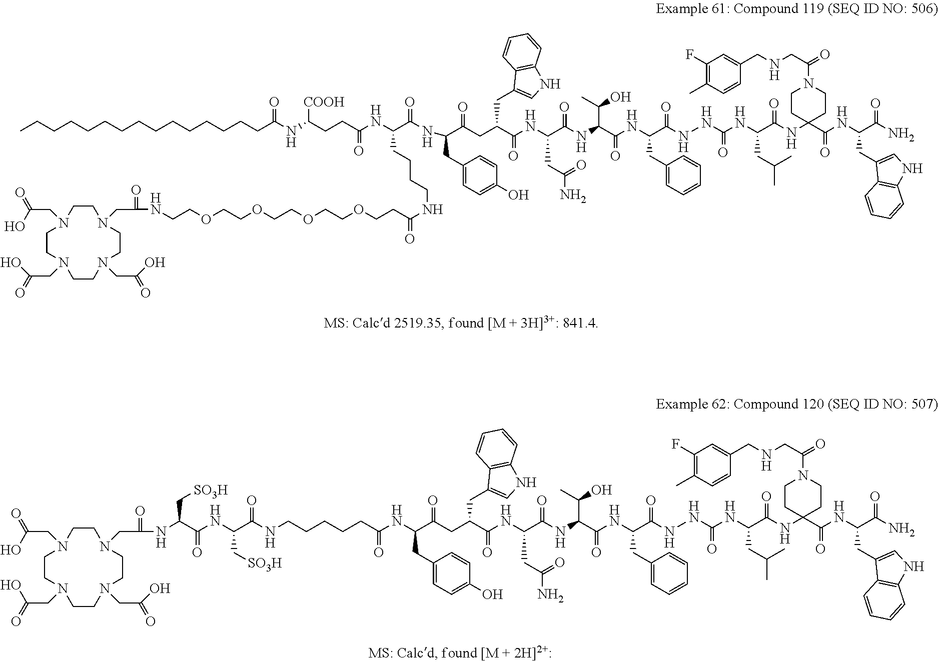

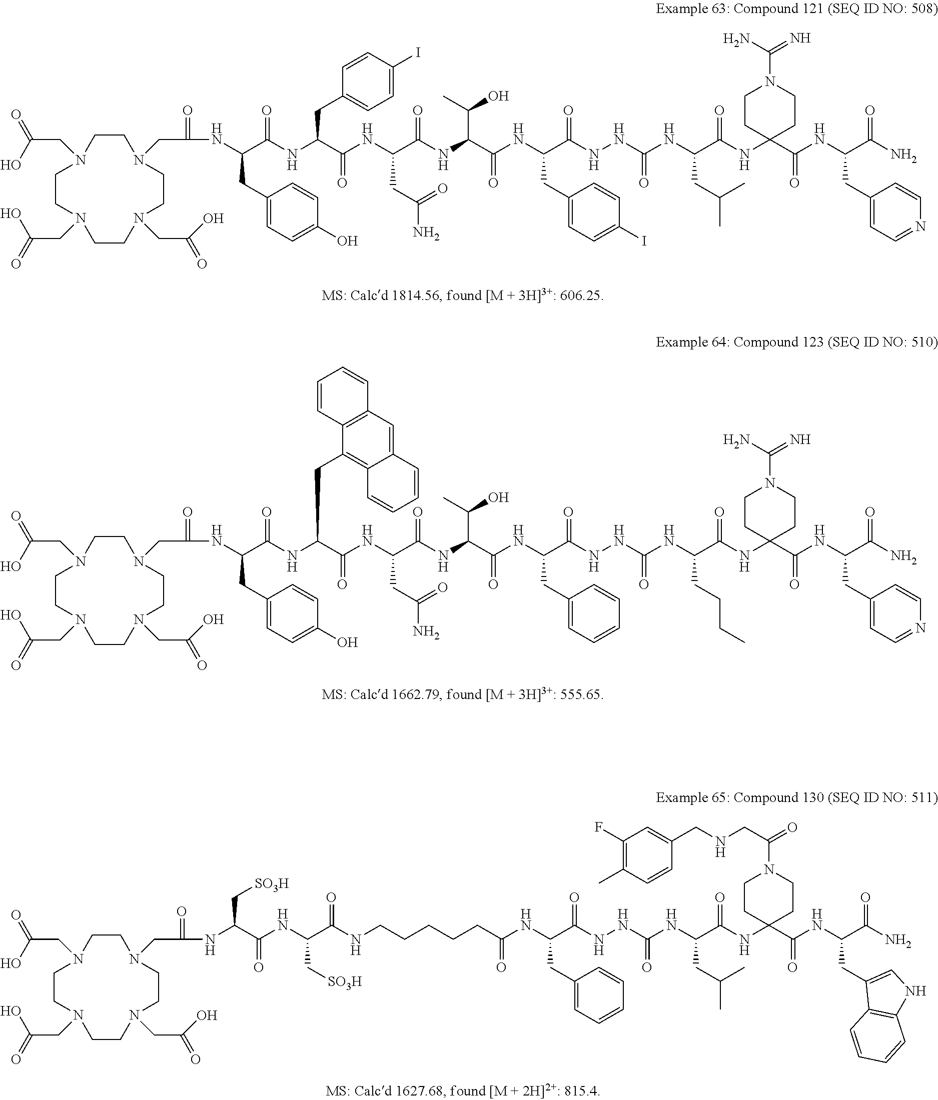

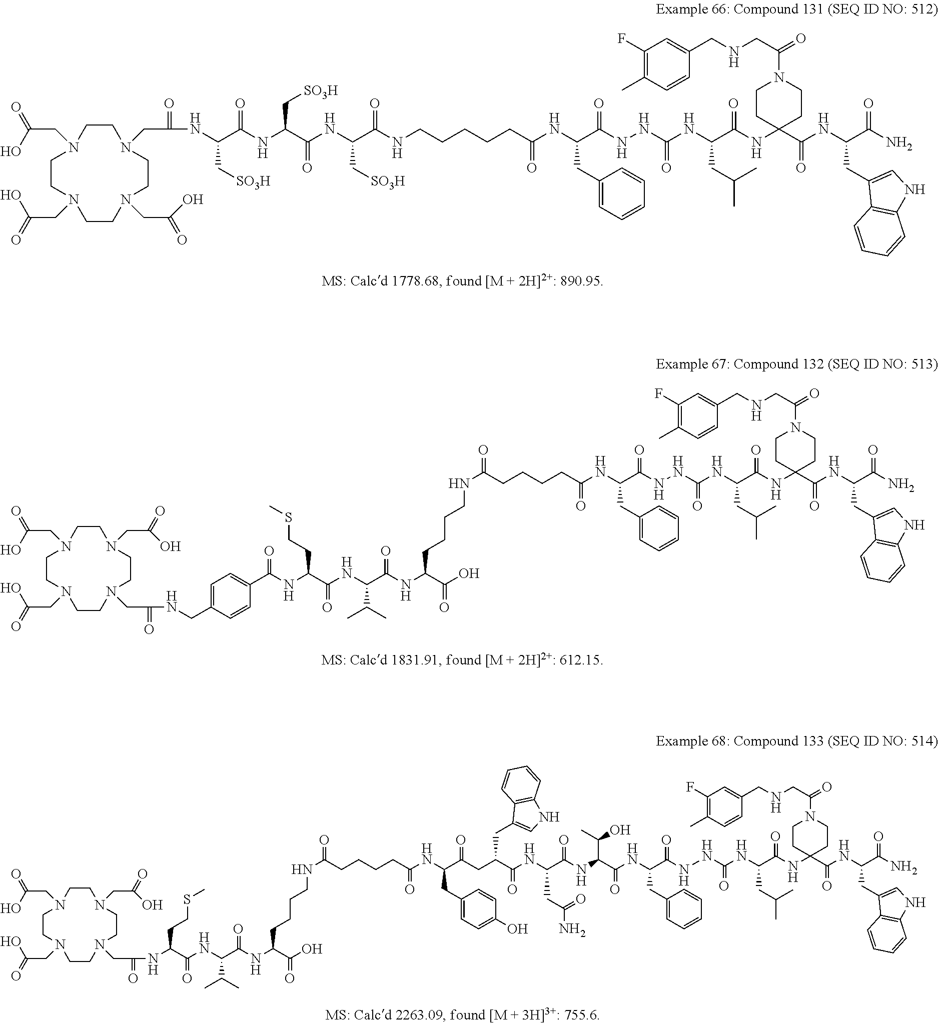

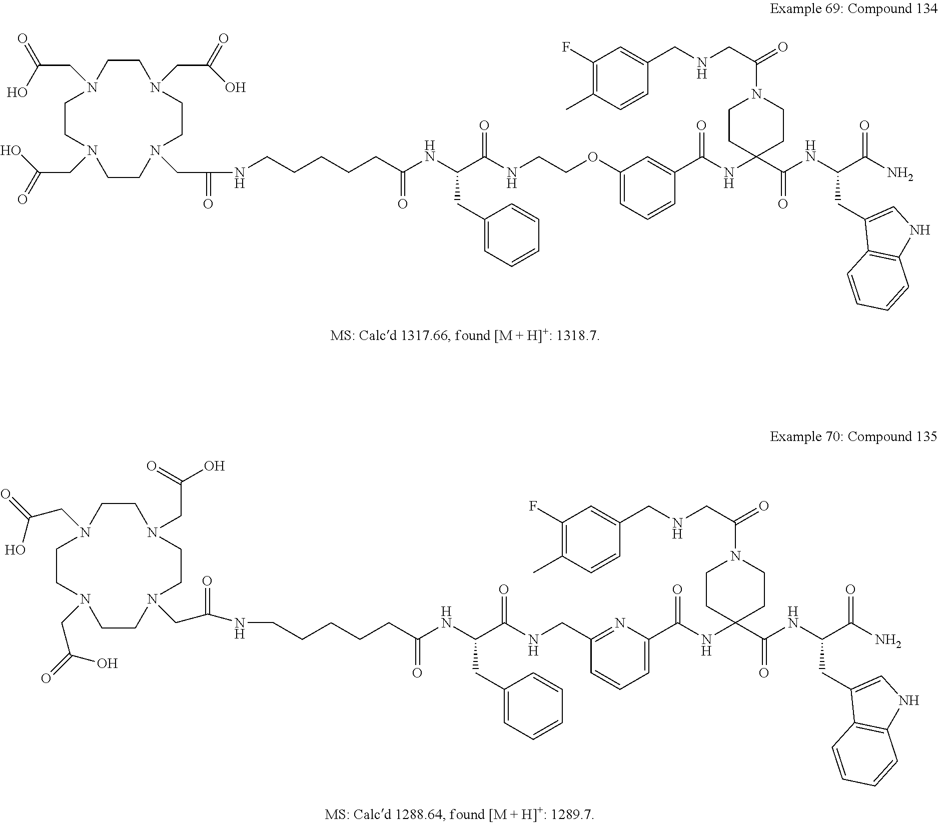 [SMILES: Cc1ccc(CNCC(=O)N2CCC(NC(=O)c3cccc(OCCNC(=O)[C@H](Cc4ccccc4)NC(=O)CCCCCNC(=O)CN4CCN(CC(=O)O)CCN(CC(=O)O)CCN(CC(=O)O)CC4)c3)(C(=O)N[C@@H](Cc3c[nH]c4ccccc34)C(N)=O)CC2)cc1F ; Cc1ccc(CNCC(=O)N2CCC(NC(=O)c3cccc(CNC(=O)[C@H](Cc4ccccc4)NC(=O)CCCCCNC(=O)CN4CCN(CC(=O)O)CCN(CC(=O)O)CCN(CC(=O)O)CC4)n3)(C(=O)N[C@@H](Cc3c[nH]c4ccccc34)C(N)=O)CC2)cc1F]

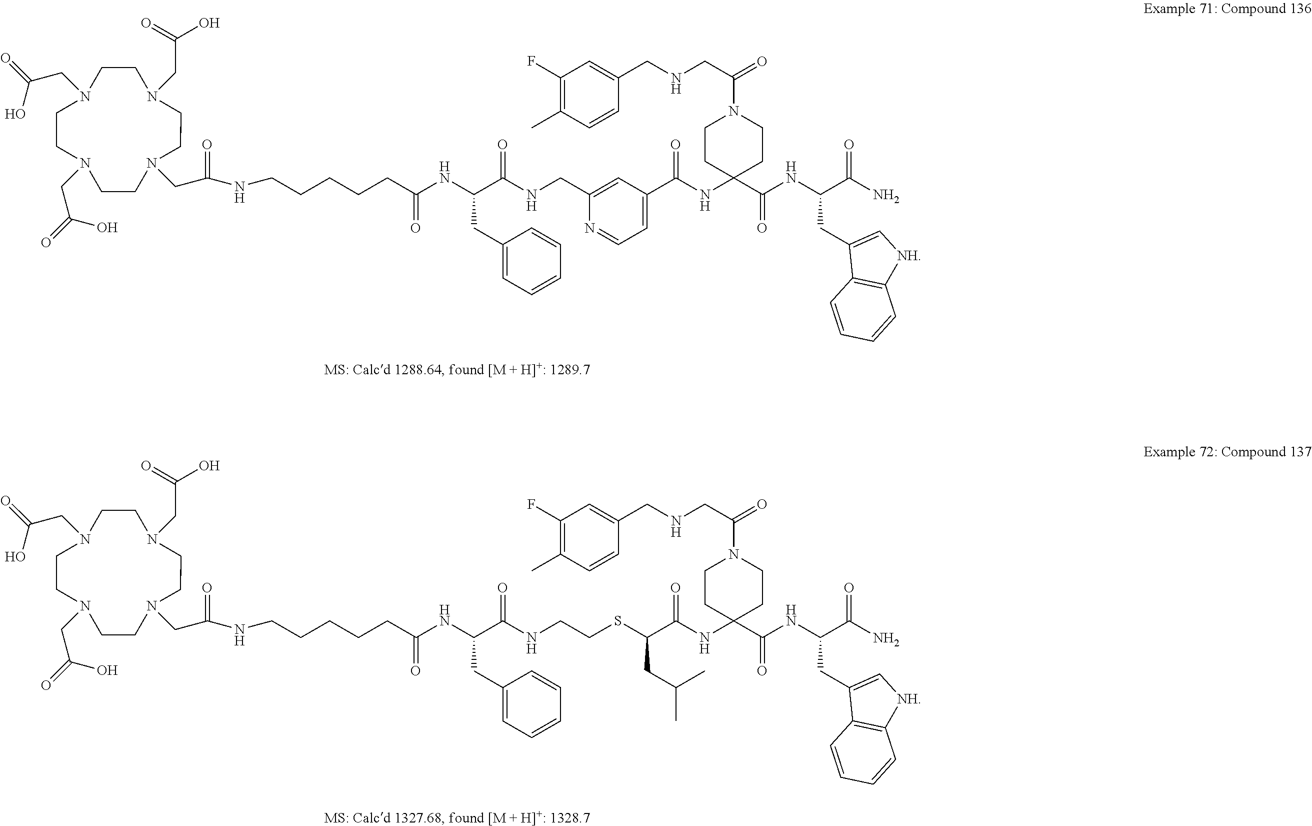 [SMILES: Cc1ccc(CNCC(=O)N2CCC(NC(=O)c3ccnc(CNC(=O)[C@H](Cc4ccccc4)NC(=O)CCCCCNC(=O)CN4CCN(CC(=O)O)CCN(CC(=O)O)CCN(CC(=O)O)CC4)c3)(C(=O)N[C@@H](Cc3c[nH]c4ccccc34)C(N)=O)CC2)cc1F ; Cc1ccc(CNCC(=O)N2CCC(NC(=O)[C@@H](CC(C)C)SCCNC(=O)[C@H](Cc3ccccc3)NC(=O)CCCCCNC(=O)CN3CCN(CC(=O)O)CCN(CC(=O)O)CCN(CC(=O)O)CC3)(C(=O)N[C@@H](Cc3c[nH]c4ccccc34)C(N)=O)CC2)cc1F]

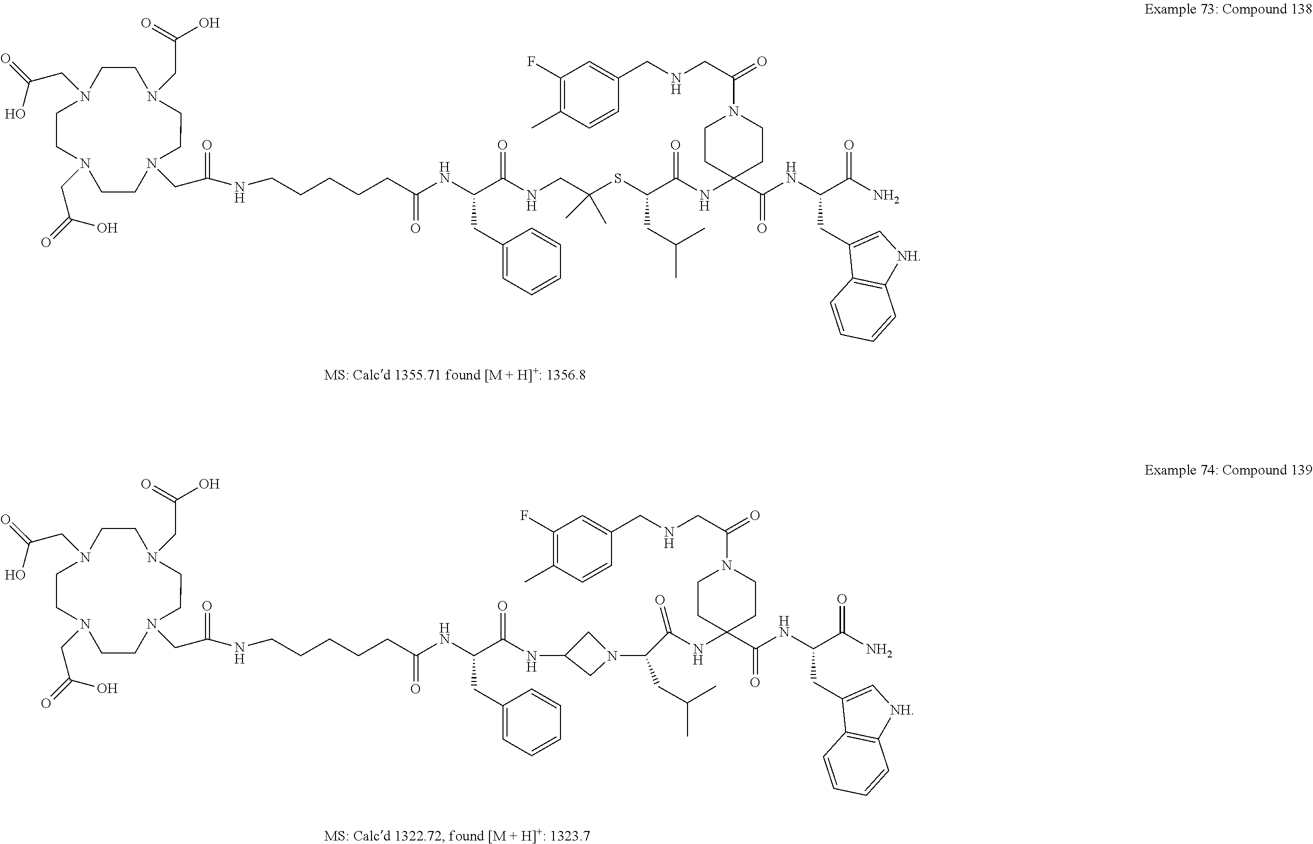 [SMILES: Cc1ccc(CNCC(=O)N2CCC(NC(=O)[C@H](CC(C)C)SC(C)(C)CNC(=O)[C@H](Cc3ccccc3)NC(=O)CCCCCNC(=O)CN3CCN(CC(=O)O)CCN(CC(=O)O)CCN(CC(=O)O)CC3)(C(=O)N[C@@H](Cc3c[nH]c4ccccc34)C(N)=O)CC2)cc1F ; Cc1ccc(CNCC(=O)N2CCC(NC(=O)[C@H](CC(C)C)N3CC(NC(=O)[C@H](Cc4ccccc4)NC(=O)CCCCCNC(=O)CN4CCN(CC(=O)O)CCN(CC(=O)O)CCN(CC(=O)O)CC4)C3)(C(=O)N[C@@H](Cc3c[nH]c4ccccc34)C(N)=O)CC2)cc1F]

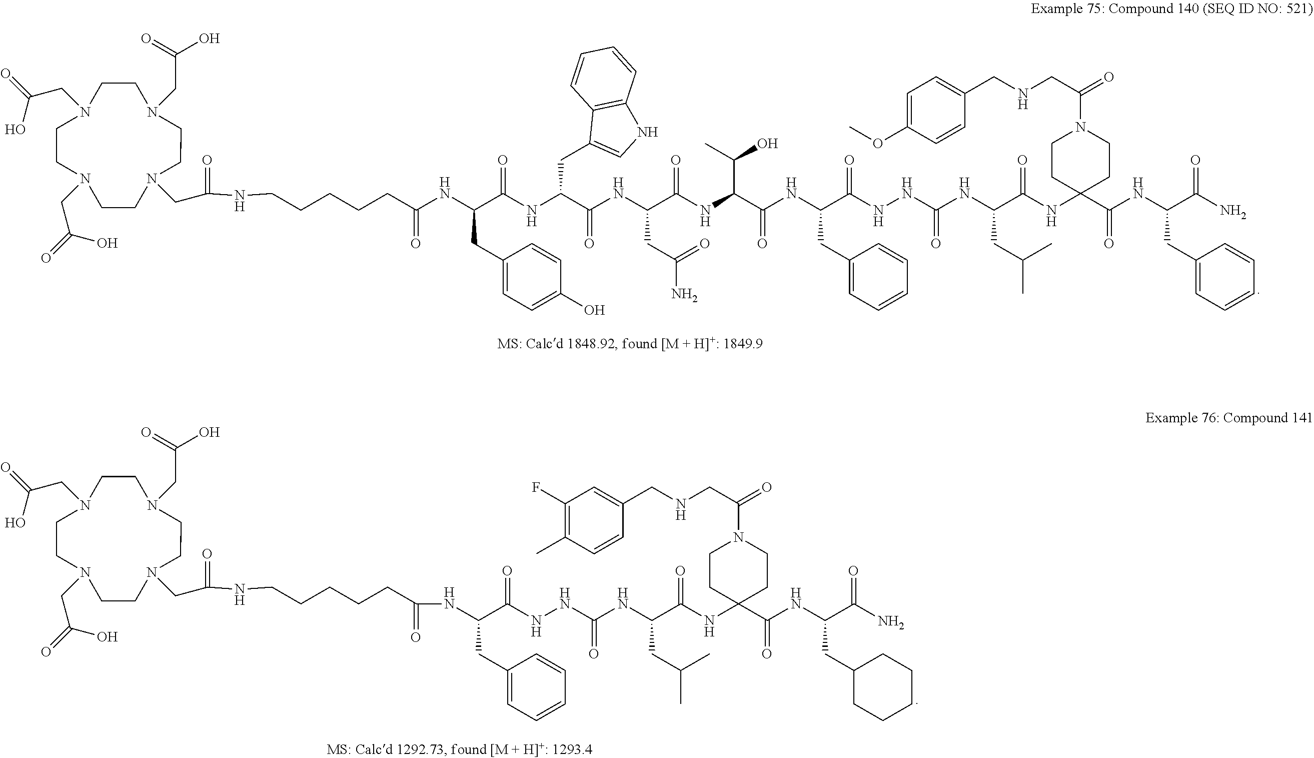 [SMILES: COc1ccc(CNCC(=O)N2CCC(NC(=O)[C@H](CC(C)C)NC(=O)NNC(=O)[C@H](Cc3ccccc3)NC(=O)[C@@H](NC(=O)[C@H](CC(N)=O)NC(=O)[C@@H](Cc3c[nH]c4ccccc34)NC(=O)[C@@H](Cc3ccc(O)cc3)NC(=O)CCCCCNC(=O)CN3CCN(CC(=O)O)CCN(CC(=O)O)CCN(CC(=O)O)CC3)[C@@H](C)O)(C(=O)N[C@@H](Cc3ccccc3)C(N)=O)CC2)cc1 ; Cc1ccc(CNCC(=O)N2CCC(NC(=O)[C@H](CC(C)C)NC(=O)NNC(=O)[C@H](Cc3ccccc3)NC(=O)CCCCCNC(=O)CN3CCN(CC(=O)O)CCN(CC(=O)O)CCN(CC(=O)O)CC3)(C(=O)N[C@@H](CC3CCCCC3)C(N)=O)CC2)cc1F]

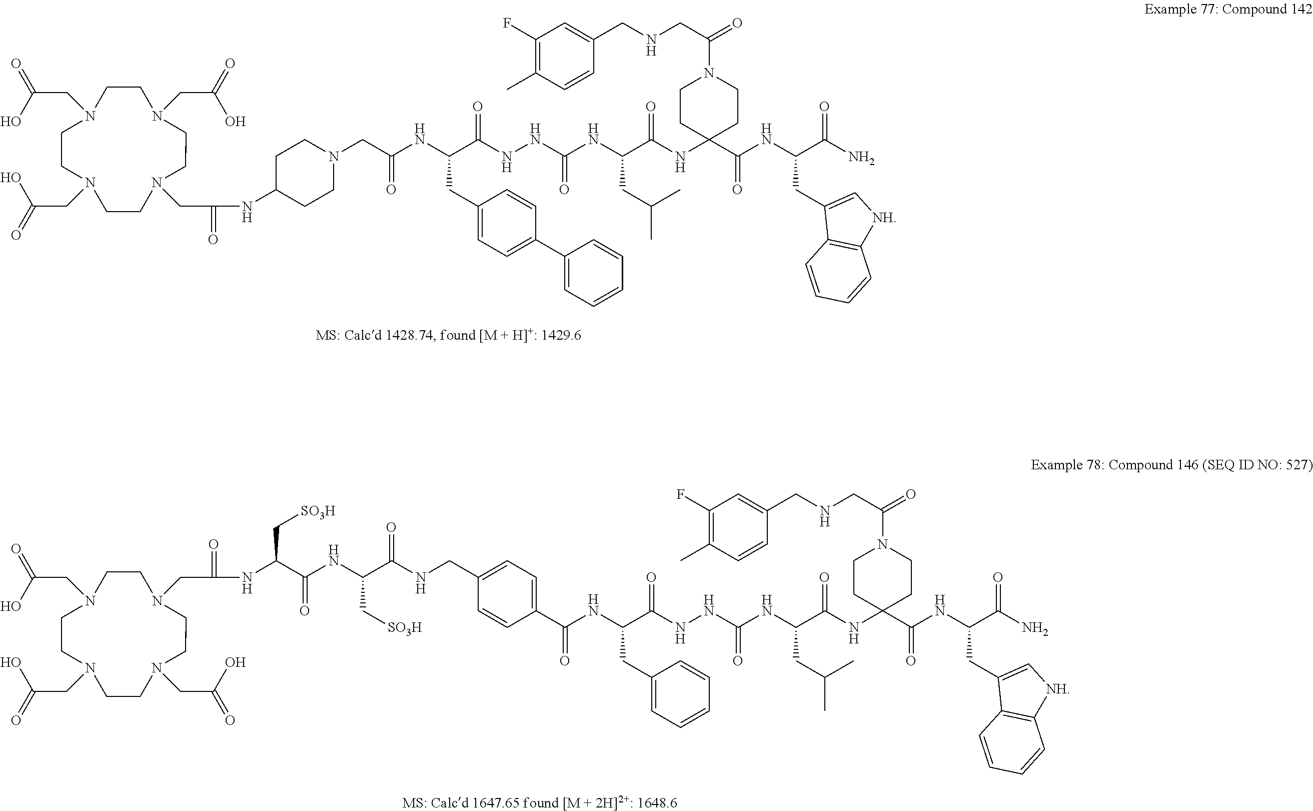 [SMILES: Cc1ccc(CNCC(=O)N2CCC(NC(=O)[C@H](CC(C)C)NC(=O)NNC(=O)[C@H](Cc3ccc(-c4ccccc4)cc3)NC(=O)CN3CCC(NC(=O)CN4CCN(CC(=O)O)CCN(CC(=O)O)CCN(CC(=O)O)CC4)CC3)(C(=O)N[C@@H](Cc3c[nH]c4ccccc34)C(N)=O)CC2)cc1F ; Cc1ccc(CNCC(=O)N2CCC(NC(=O)[C@H](CC(C)C)NC(=O)NNC(=O)[C@H](Cc3ccccc3)NC(=O)c3ccc(CNC(=O)[C@H](CS(=O)(=O)O)NC(=O)[C@H](CS(=O)(=O)O)NC(=O)CN4CCN(CC(=O)O)CCN(CC(=O)O)CCN(CC(=O)O)CC4)cc3)(C(=O)N[C@@H](Cc3c[nH]c4ccccc34)C(N)=O)CC2)cc1F]

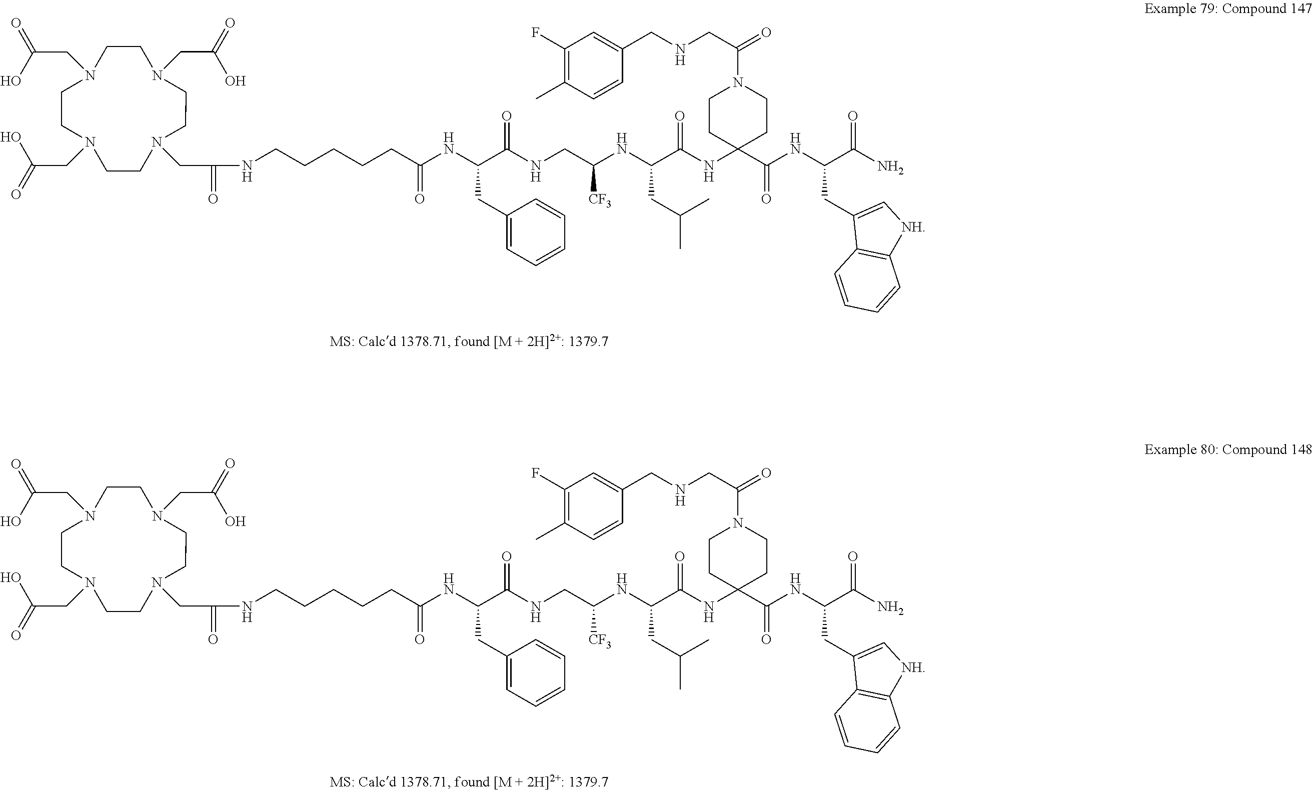 [SMILES: Cc1ccc(CNCC(=O)N2CCC(NC(=O)[C@H](CC(C)C)N[C@H](CNC(=O)[C@H](Cc3ccccc3)NC(=O)CCCCCNC(=O)CN3CCN(CC(=O)O)CCN(CC(=O)O)CCN(CC(=O)O)CC3)C(F)(F)F)(C(=O)N[C@@H](Cc3c[nH]c4ccccc34)C(N)=O)CC2)cc1F ; Cc1ccc(CNCC(=O)N2CCC(NC(=O)[C@H](CC(C)C)N[C@@H](CNC(=O)[C@H](Cc3ccccc3)NC(=O)CCCCCNC(=O)CN3CCN(CC(=O)O)CCN(CC(=O)O)CCN(CC(=O)O)CC3)C(F)(F)F)(C(=O)N[C@@H](Cc3c[nH]c4ccccc34)C(N)=O)CC2)cc1F]

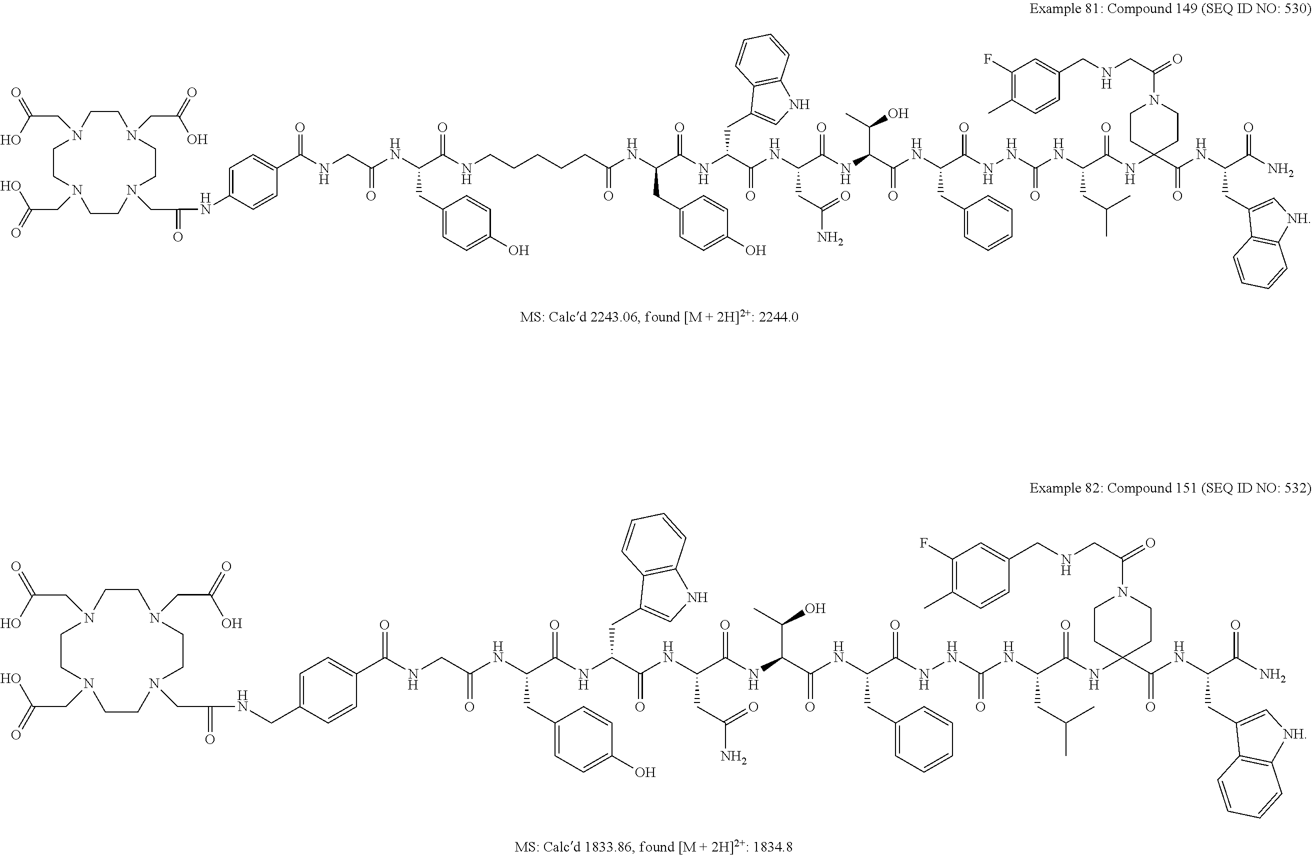

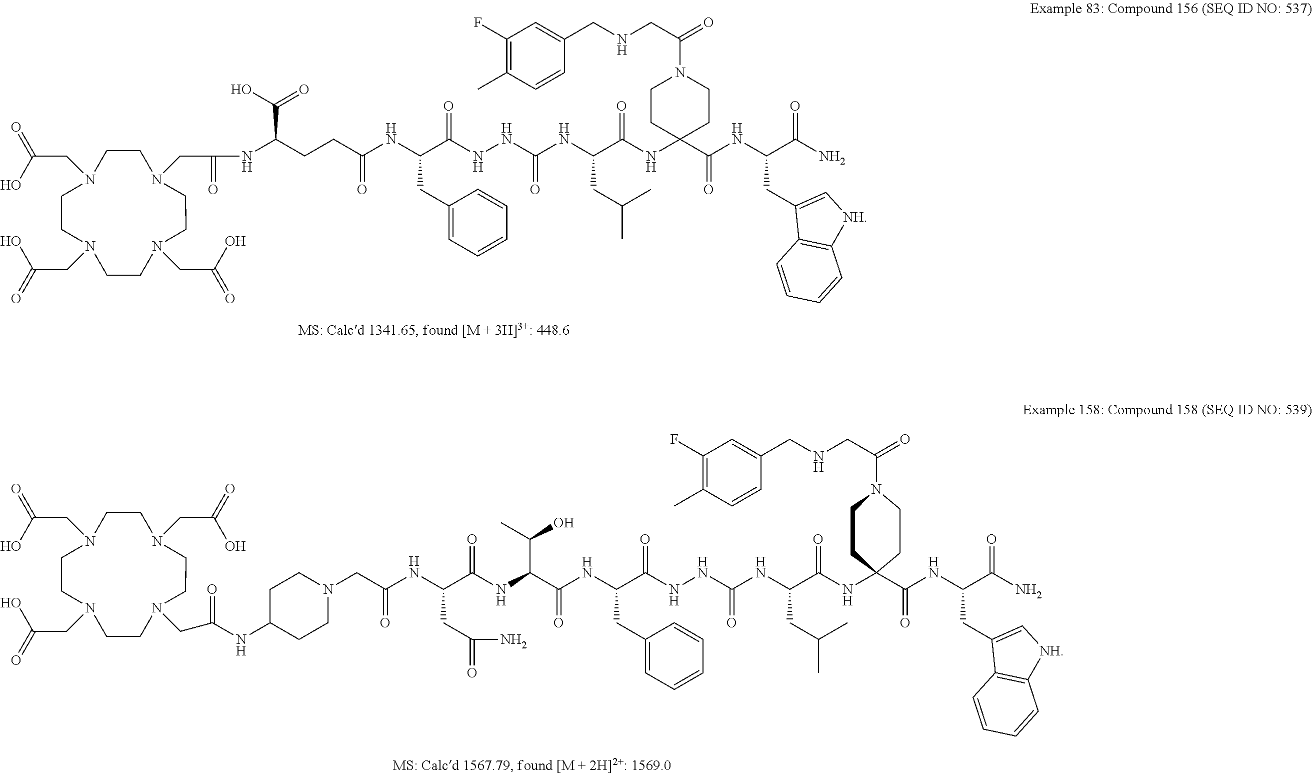

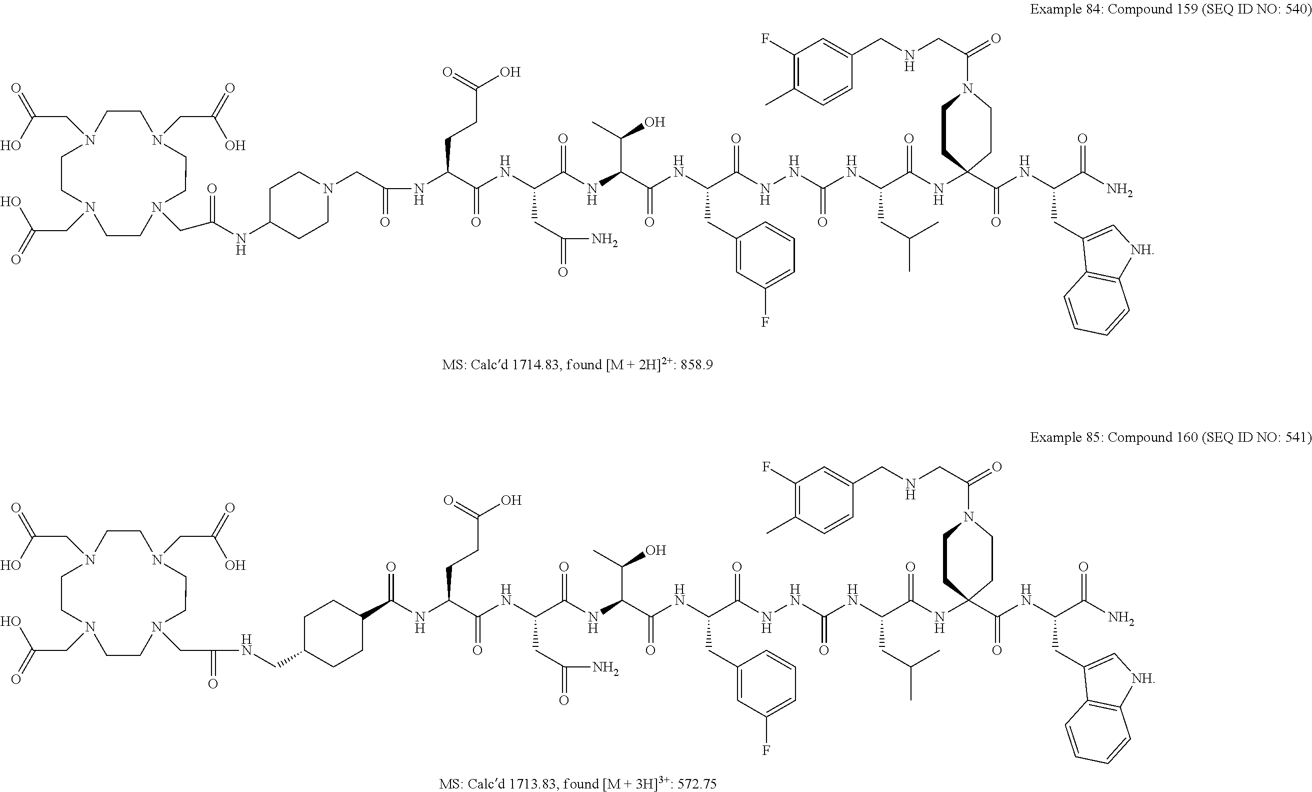

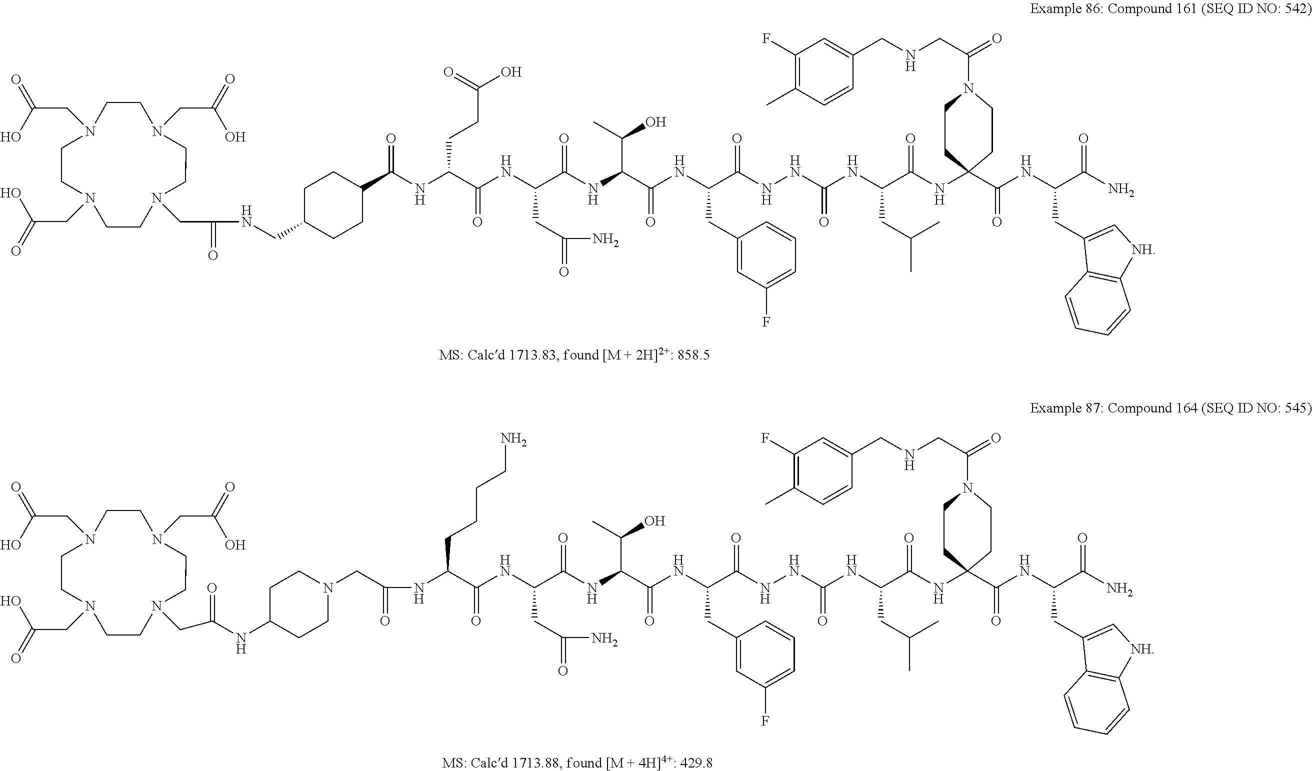

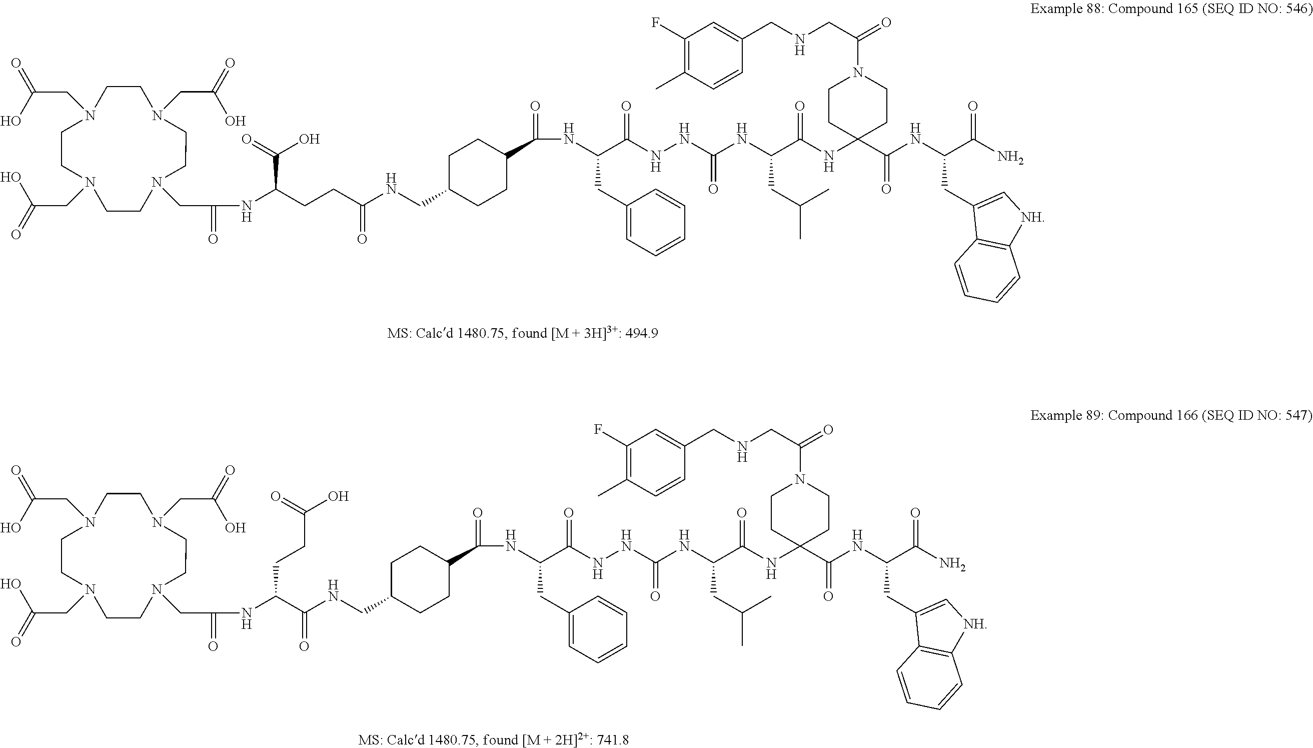

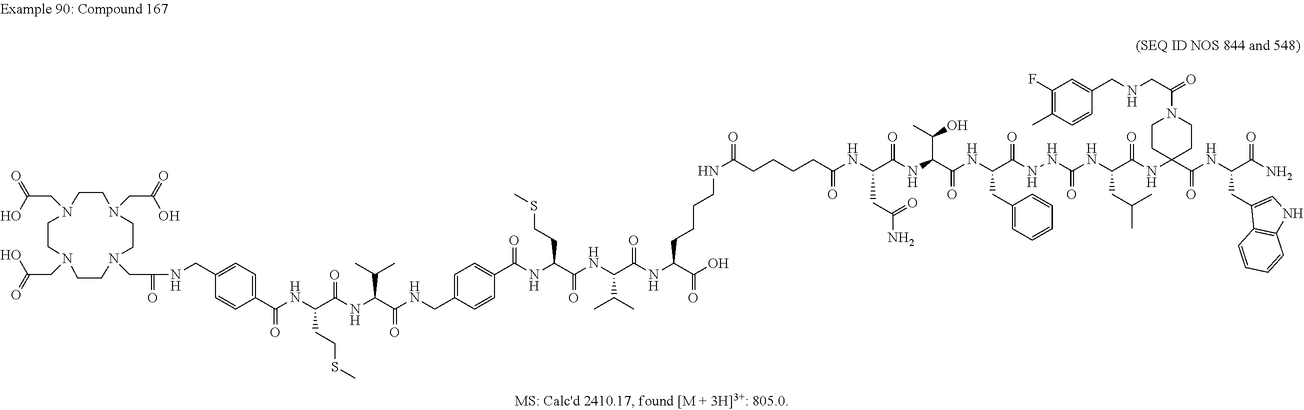 [SMILES: CSCC[C@H](NC(=O)c1ccc(CNC(=O)CN2CCN(CC(=O)O)CCN(CC(=O)O)CCN(CC(=O)O)CC2)cc1)C(=O)N[C@H](C(=O)NCc1ccc(C(=O)N[C@@H](CCSC)C(=O)N[C@H](C(=O)N[C@@H](CCCCNC(=O)CCCCC(=O)N[C@@H](CC(N)=O)C(=O)N[C@H](C(=O)N[C@@H](Cc2ccccc2)C(=O)NNC(=O)N[C@@H](CC(C)C)C(=O)NC2(C(=O)N[C@@H](Cc3c[nH]c4ccccc34)C(N)=O)CCN(C(=O)CNCc3ccc(C)c(F)c3)CC2)[C@@H](C)O)C(=O)O)C(C)C)cc1)C(C)C]

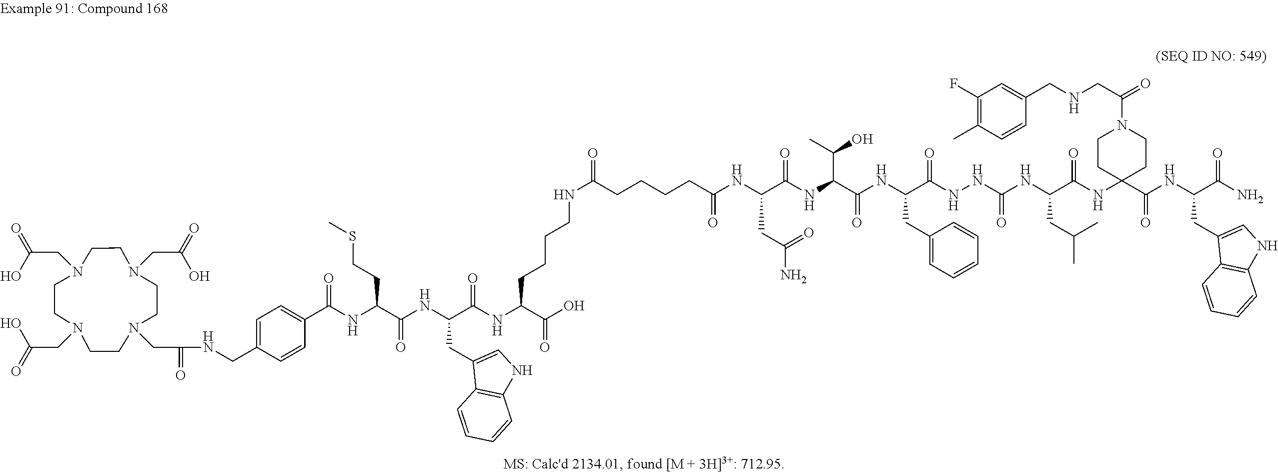 [SMILES: CSCC[C@H](NC(=O)c1ccc(CNC(=O)CN2CCN(CC(=O)O)CCN(CC(=O)O)CCN(CC(=O)O)CC2)cc1)C(=O)N[C@@H](Cc1c[nH]c2ccccc12)C(=O)N[C@@H](CCCCNC(=O)CCCCC(=O)N[C@@H](CC(N)=O)C(=O)N[C@H](C(=O)N[C@@H](Cc1ccccc1)C(=O)NNC(=O)N[C@@H](CC(C)C)C(=O)NC1(C(=O)N[C@@H](Cc2c[nH]c3ccccc23)C(N)=O)CCN(C(=O)CNCc2ccc(C)c(F)c2)CC1)[C@@H](C)O)C(=O)O]

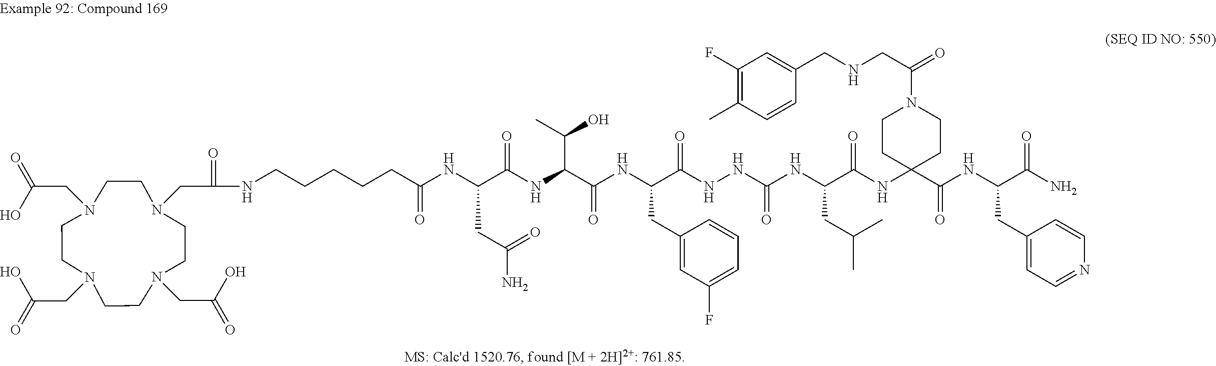 [SMILES: Cc1ccc(CNCC(=O)N2CCC(NC(=O)[C@H](CC(C)C)NC(=O)NNC(=O)[C@H](Cc3cccc(F)c3)NC(=O)[C@@H](NC(=O)[C@H](CC(N)=O)NC(=O)CCCCCNC(=O)CN3CCN(CC(=O)O)CCN(CC(=O)O)CCN(CC(=O)O)CC3)[C@@H](C)O)(C(=O)N[C@@H](Cc3ccncc3)C(N)=O)CC2)cc1F]

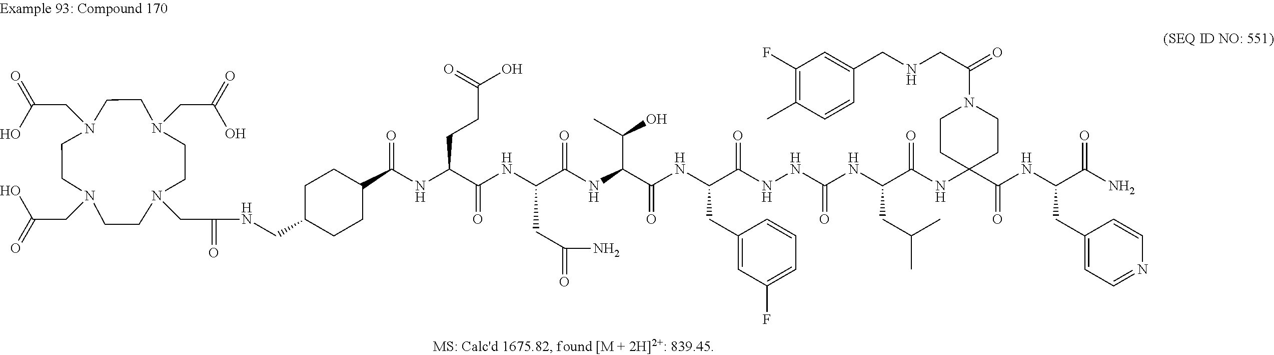

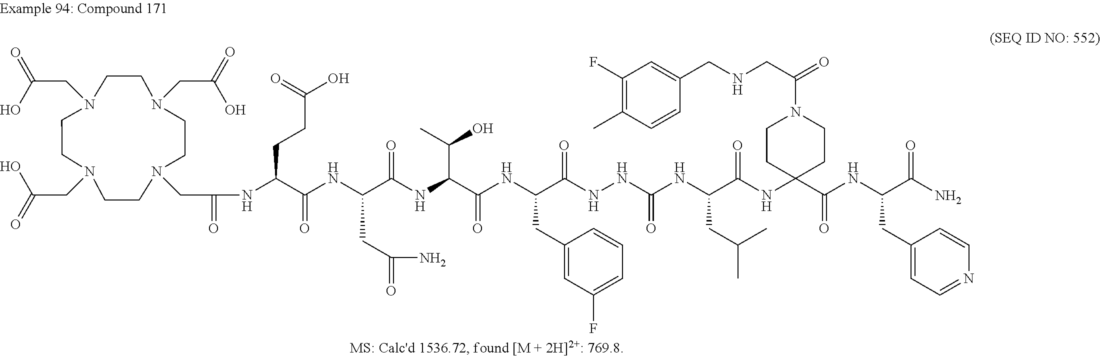 [SMILES: Cc1ccc(CNCC(=O)N2CCC(NC(=O)[C@H](CC(C)C)NC(=O)NNC(=O)[C@H](Cc3cccc(F)c3)NC(=O)[C@@H](NC(=O)[C@H](CC(N)=O)NC(=O)[C@H](CCC(=O)O)NC(=O)CN3CCN(CC(=O)O)CCN(CC(=O)O)CCN(CC(=O)O)CC3)[C@@H](C)O)(C(=O)N[C@@H](Cc3ccncc3)C(N)=O)CC2)cc1F]

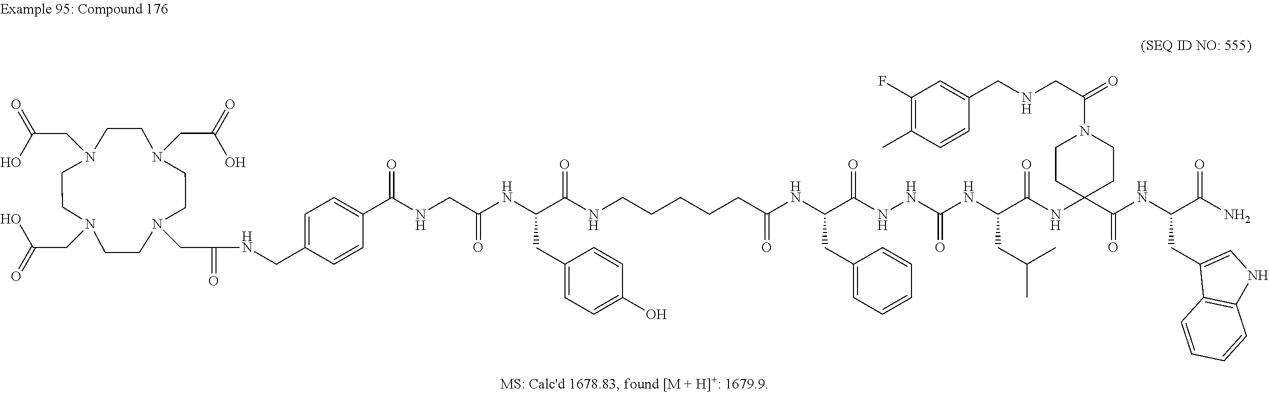 [SMILES: Cc1ccc(CNCC(=O)N2CCC(NC(=O)[C@H](CC(C)C)NC(=O)NNC(=O)[C@H](Cc3ccccc3)NC(=O)CCCCCNC(=O)[C@H](Cc3ccc(O)cc3)NC(=O)CNC(=O)c3ccc(CNC(=O)CN4CCN(CC(=O)O)CCN(CC(=O)O)CCN(CC(=O)O)CC4)cc3)(C(=O)N[C@@H](Cc3c[nH]c4ccccc34)C(N)=O)CC2)cc1F]

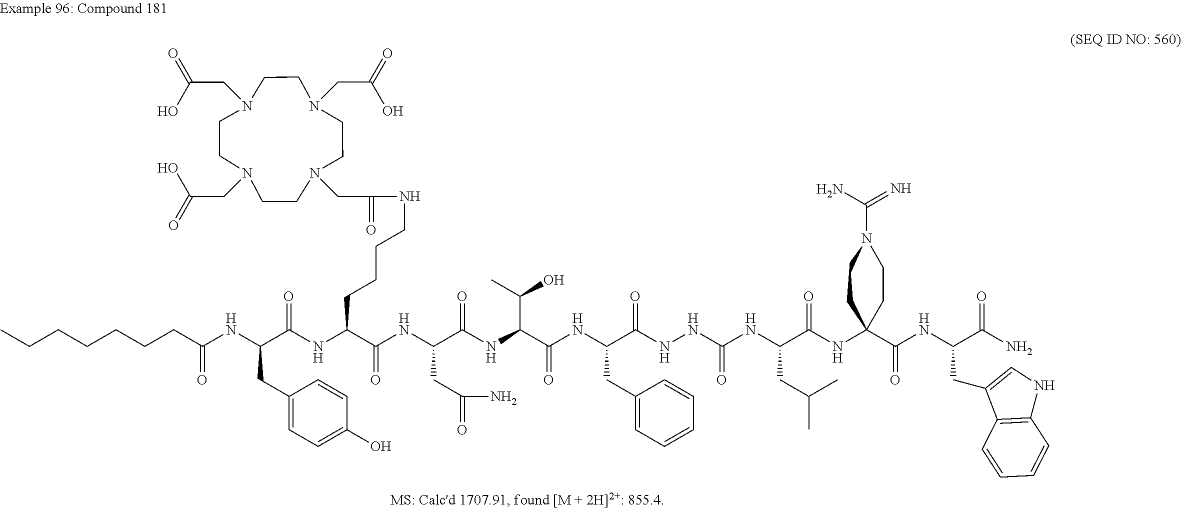

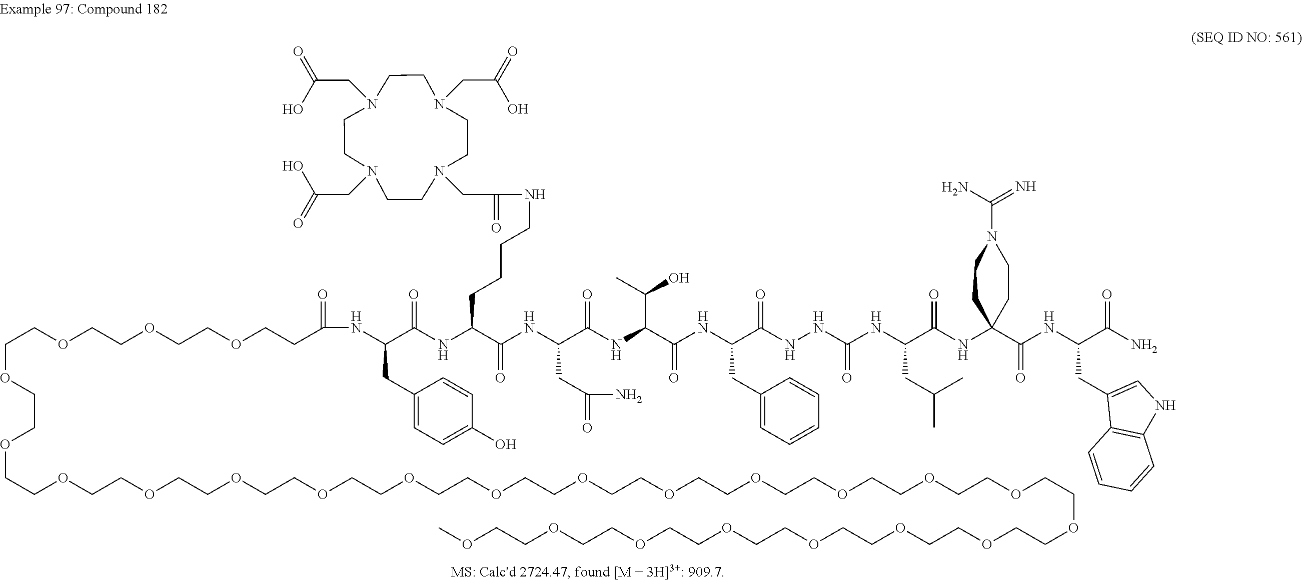

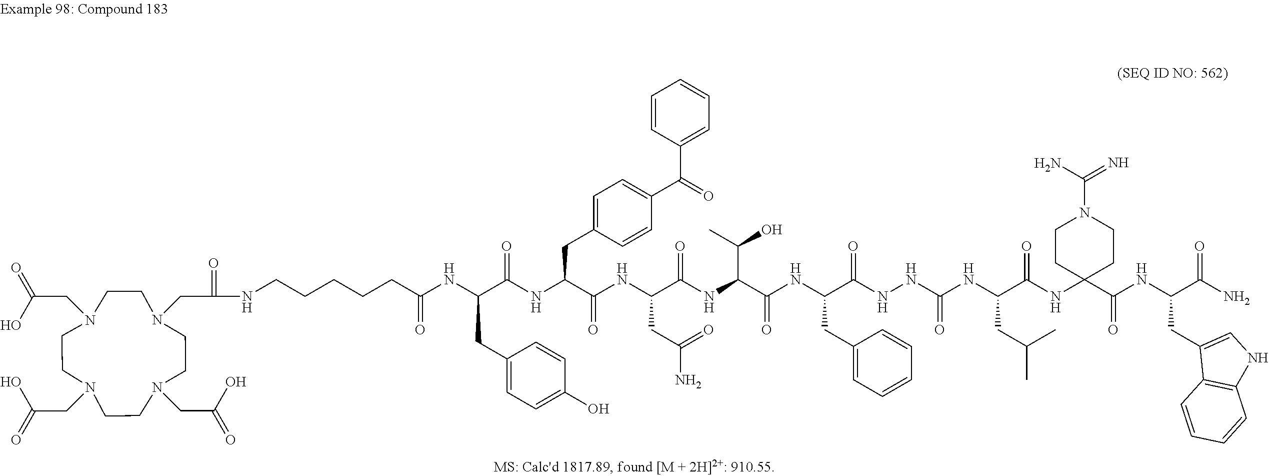 [SMILES: CC(C)C[C@H](NC(=O)NNC(=O)[C@H](Cc1ccccc1)NC(=O)[C@@H](NC(=O)[C@H](CC(N)=O)NC(=O)[C@H](Cc1ccc(C(=O)c2ccccc2)cc1)NC(=O)[C@@H](Cc1ccc(O)cc1)NC(=O)CCCCCNC(=O)CN1CCN(CC(=O)O)CCN(CC(=O)O)CCN(CC(=O)O)CC1)[C@@H](C)O)C(=O)NC1(C(=O)N[C@@H](Cc2c[nH]c3ccccc23)C(N)=O)CCN(C(=N)N)CC1]

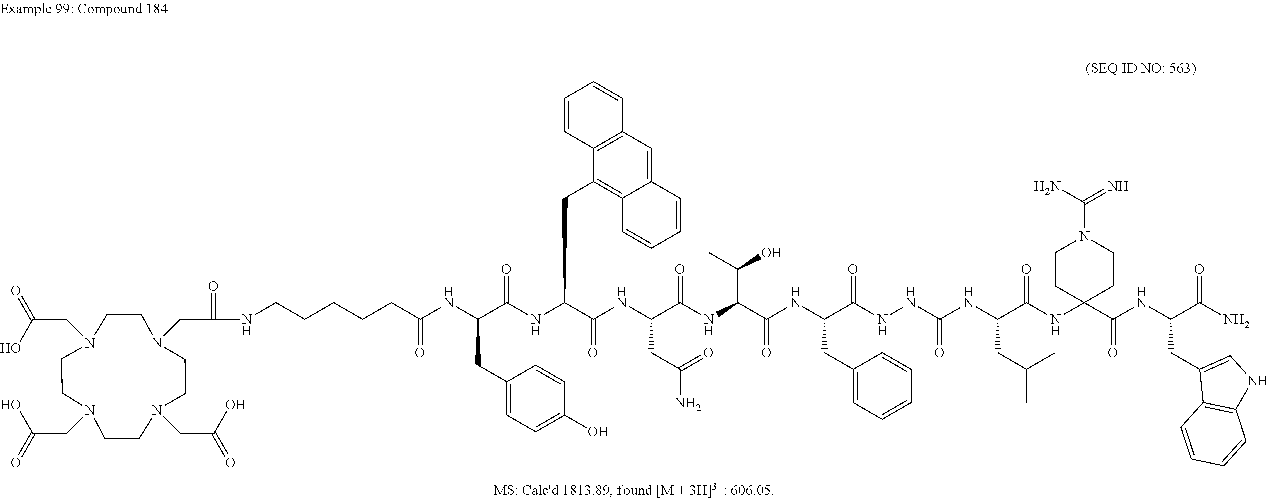 [SMILES: CC(C)C[C@H](NC(=O)NNC(=O)[C@H](Cc1ccccc1)NC(=O)[C@@H](NC(=O)[C@H](CC(N)=O)NC(=O)[C@H](Cc1c2ccccc2cc2ccccc12)NC(=O)[C@@H](Cc1ccc(O)cc1)NC(=O)CCCCCNC(=O)CN1CCN(CC(=O)O)CCN(CC(=O)O)CCN(CC(=O)O)CC1)[C@@H](C)O)C(=O)NC1(C(=O)N[C@@H](Cc2c[nH]c3ccccc23)C(N)=O)CCN(C(=N)N)CC1]

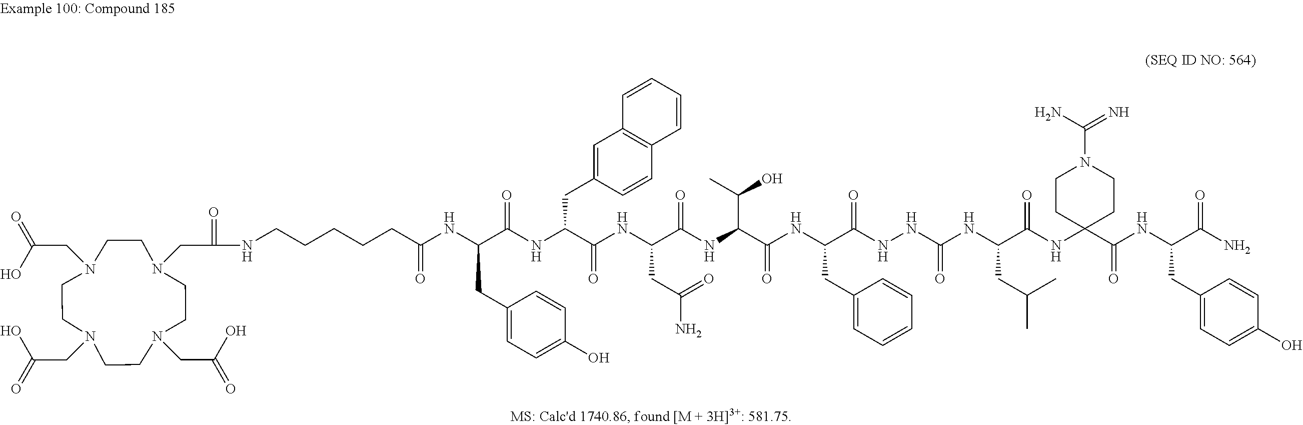 [SMILES: CC(C)C[C@H](NC(=O)NNC(=O)[C@H](Cc1ccccc1)NC(=O)[C@@H](NC(=O)[C@H](CC(N)=O)NC(=O)[C@@H](Cc1ccc2ccccc2c1)NC(=O)[C@@H](Cc1ccc(O)cc1)NC(=O)CCCCCNC(=O)CN1CCN(CC(=O)O)CCN(CC(=O)O)CCN(CC(=O)O)CC1)[C@@H](C)O)C(=O)NC1(C(=O)N[C@@H](Cc2ccc(O)cc2)C(N)=O)CCN(C(=N)N)CC1]

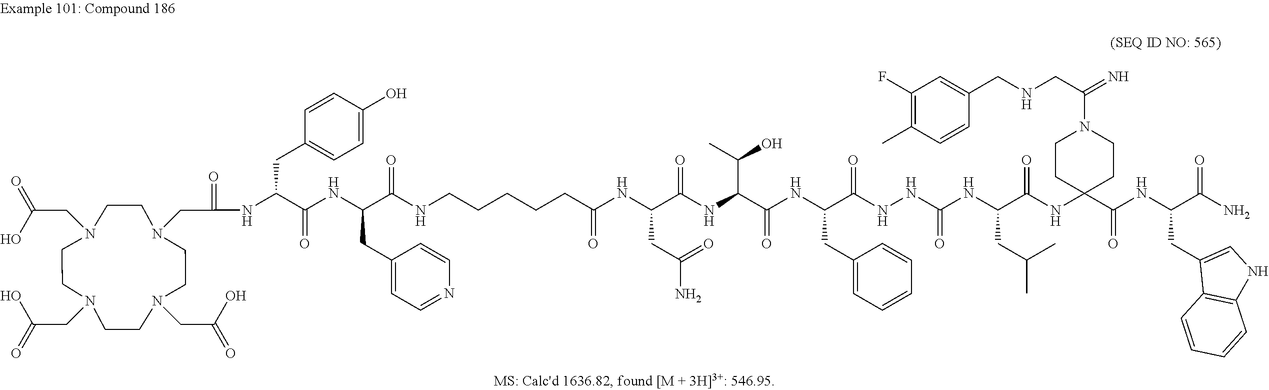 [SMILES: Cc1ccc(CNCC(=N)N2CCC(NC(=O)[C@H](CC(C)C)NC(=O)NNC(=O)[C@H](Cc3ccccc3)NC(=O)[C@@H](NC(=O)[C@H](CC(N)=O)NC(=O)CCCCCNC(=O)[C@@H](Cc3ccncc3)NC(=O)[C@@H](Cc3ccc(O)cc3)NC(=O)CN3CCN(CC(=O)O)CCN(CC(=O)O)CCN(CC(=O)O)CC3)[C@@H](C)O)(C(=O)N[C@@H](Cc3c[nH]c4ccccc34)C(N)=O)CC2)cc1F]

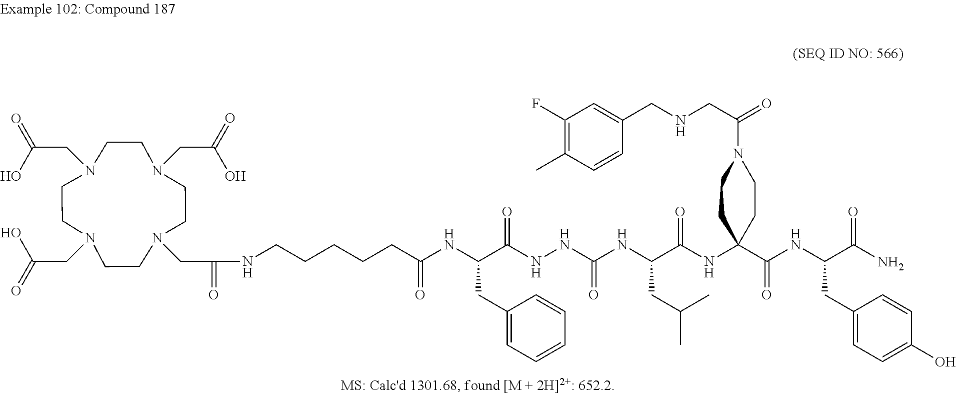

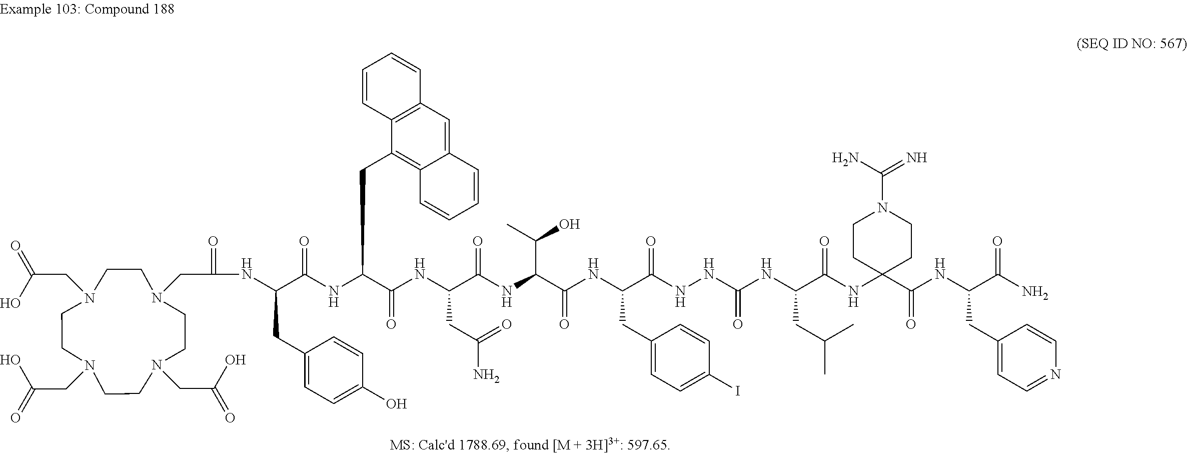 [SMILES: CC(C)C[C@H](NC(=O)NNC(=O)[C@H](Cc1ccc(I)cc1)NC(=O)[C@@H](NC(=O)[C@H](CC(N)=O)NC(=O)[C@H](Cc1c2ccccc2cc2ccccc12)NC(=O)[C@@H](Cc1ccc(O)cc1)NC(=O)CN1CCN(CC(=O)O)CCN(CC(=O)O)CCN(CC(=O)O)CC1)[C@@H](C)O)C(=O)NC1(C(=O)N[C@@H](Cc2ccncc2)C(N)=O)CCN(C(=N)N)CC1]

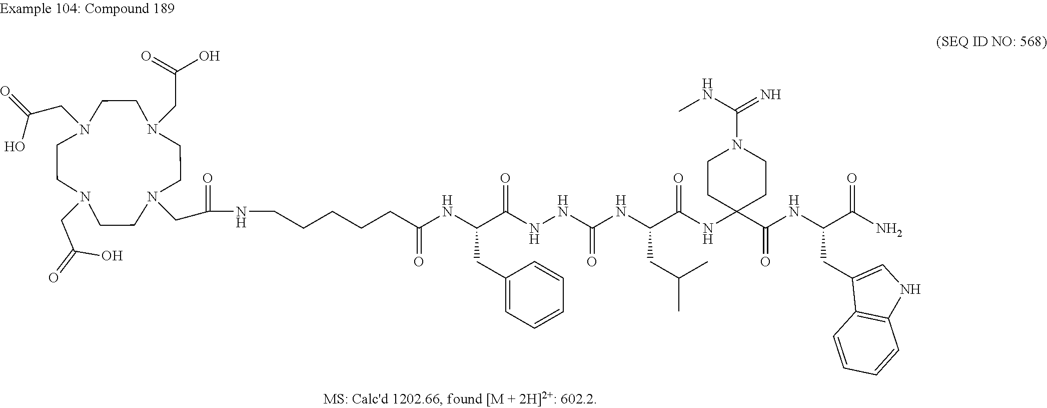 [SMILES: CNC(=N)N1CCC(NC(=O)[C@H](CC(C)C)NC(=O)NNC(=O)[C@H](Cc2ccccc2)NC(=O)CCCCCNC(=O)CN2CCN(CC(=O)O)CCN(CC(=O)O)CCN(CC(=O)O)CC2)(C(=O)N[C@@H](Cc2c[nH]c3ccccc23)C(N)=O)CC1]

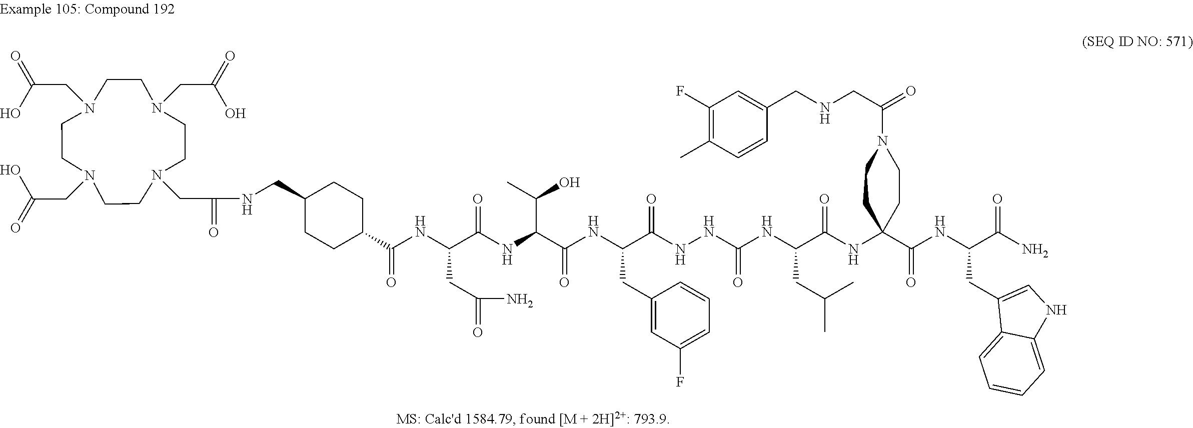

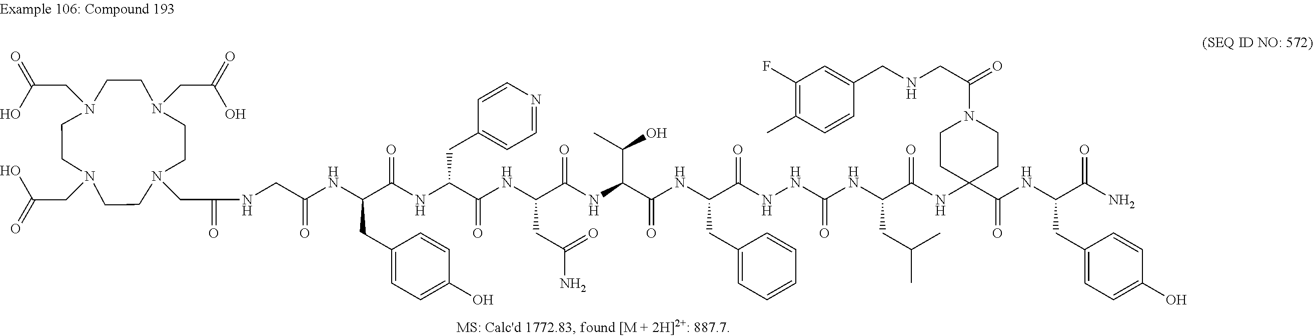 [SMILES: Cc1ccc(CNCC(=O)N2CCC(NC(=O)[C@H](CC(C)C)NC(=O)NNC(=O)[C@H](Cc3ccccc3)NC(=O)[C@@H](NC(=O)[C@H](CC(N)=O)NC(=O)[C@@H](Cc3ccncc3)NC(=O)[C@@H](Cc3ccc(O)cc3)NC(=O)CNC(=O)CN3CCN(CC(=O)O)CCN(CC(=O)O)CCN(CC(=O)O)CC3)[C@@H](C)O)(C(=O)N[C@@H](Cc3ccc(O)cc3)C(N)=O)CC2)cc1F]

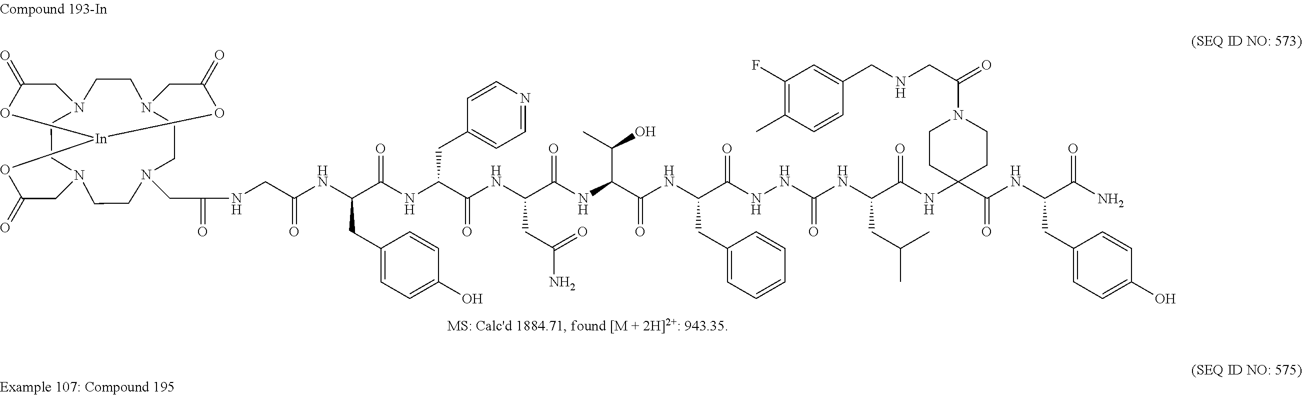

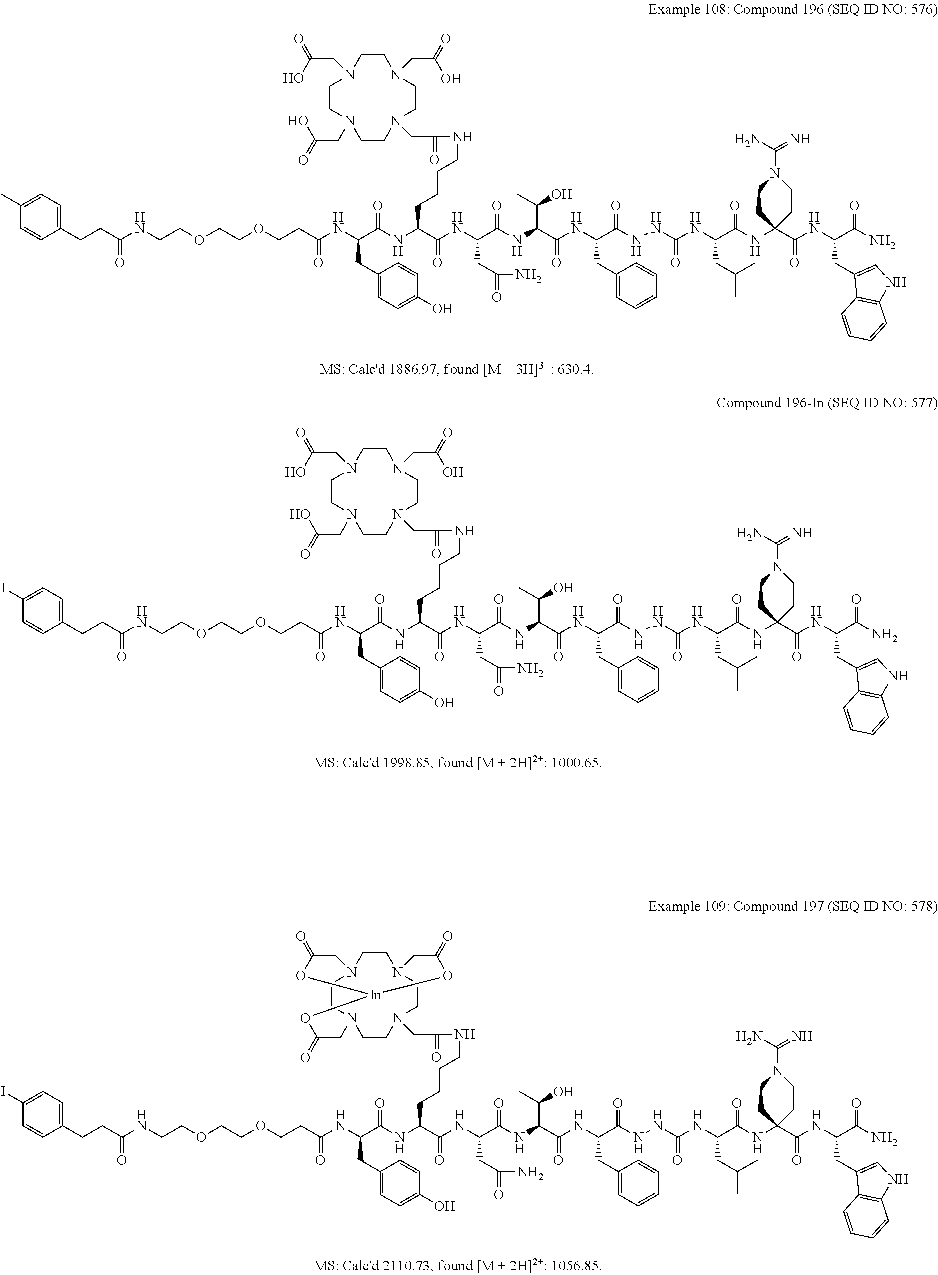

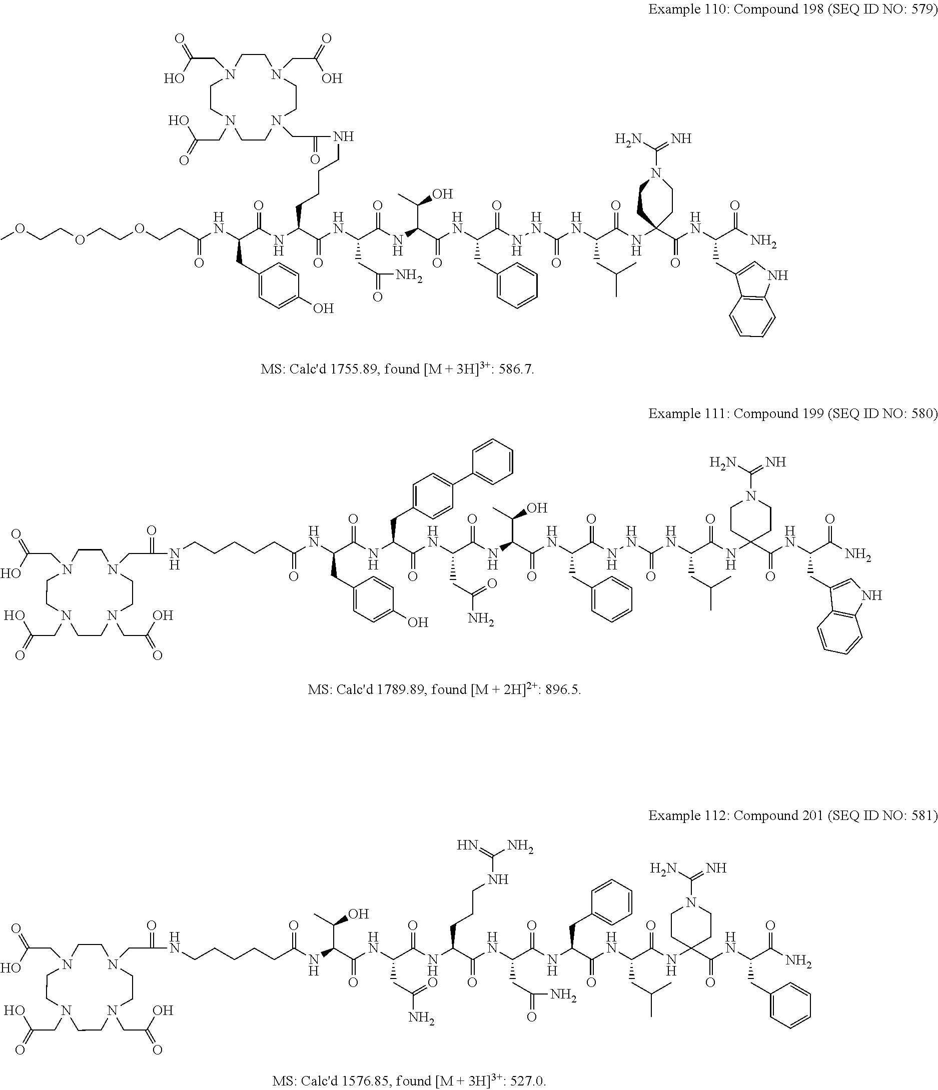

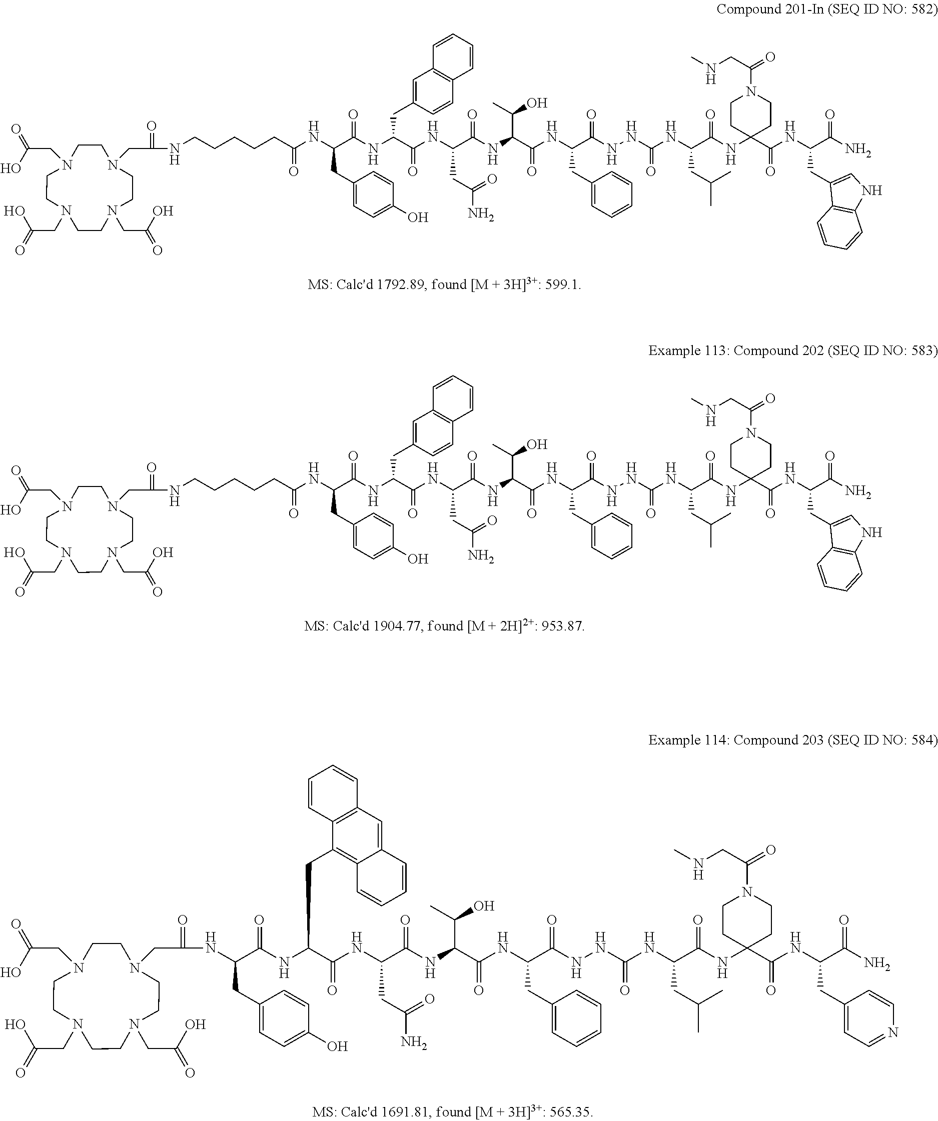

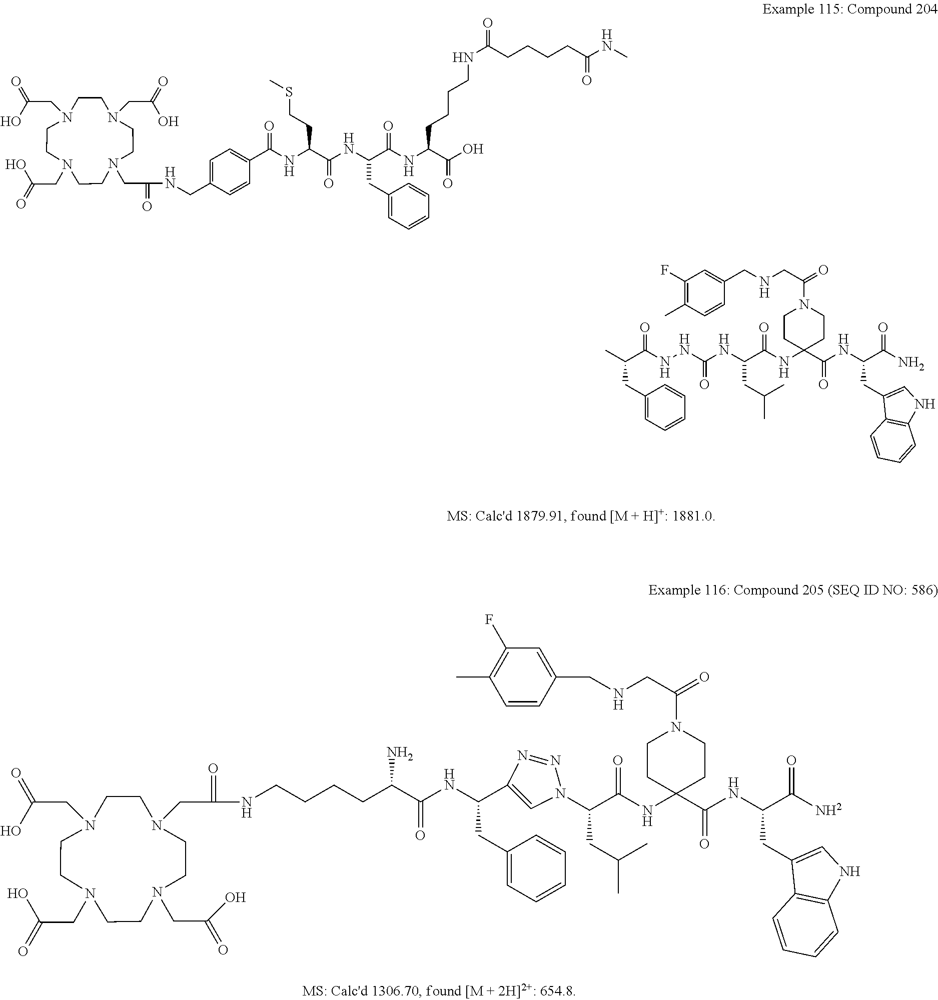

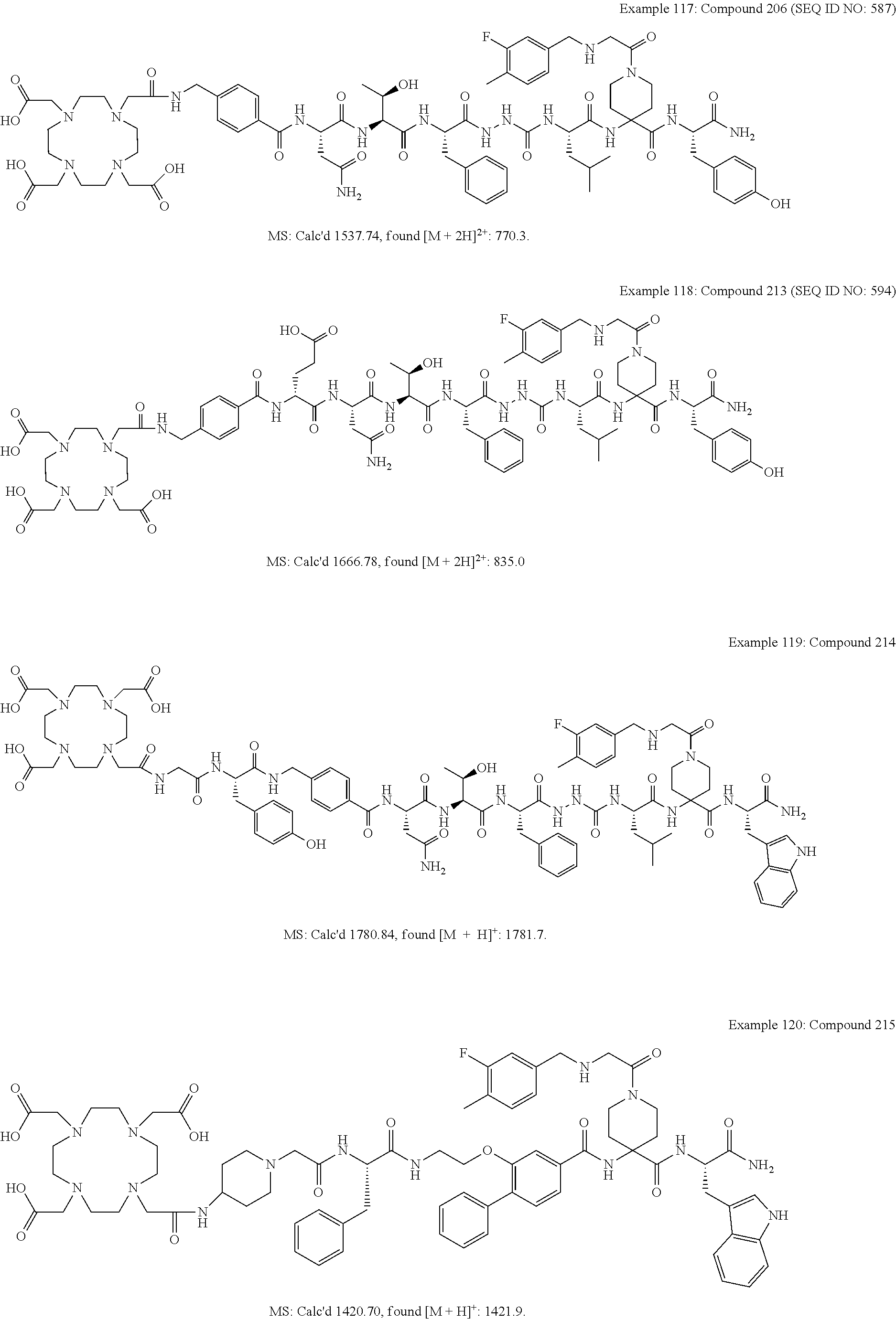

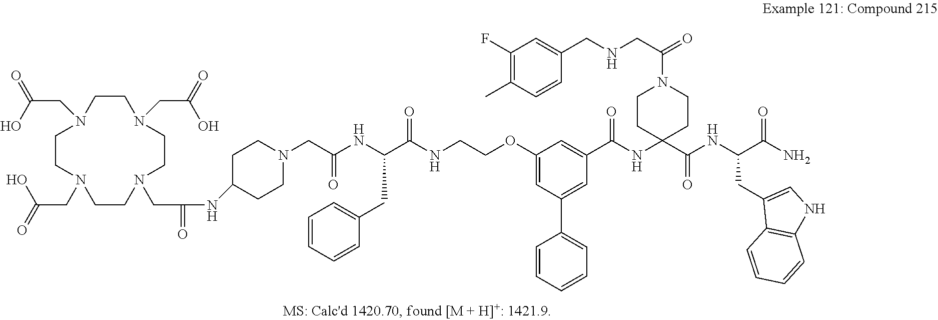 [SMILES: Cc1ccc(CNCC(=O)N2CCC(NC(=O)c3cc(OCCNC(=O)[C@H](Cc4ccccc4)NC(=O)CN4CCC(NC(=O)CN5CCN(CC(=O)O)CCN(CC(=O)O)CCN(CC(=O)O)CC5)CC4)cc(-c4ccccc4)c3)(C(=O)N[C@@H](Cc3c[nH]c4ccccc34)C(N)=O)CC2)cc1F]

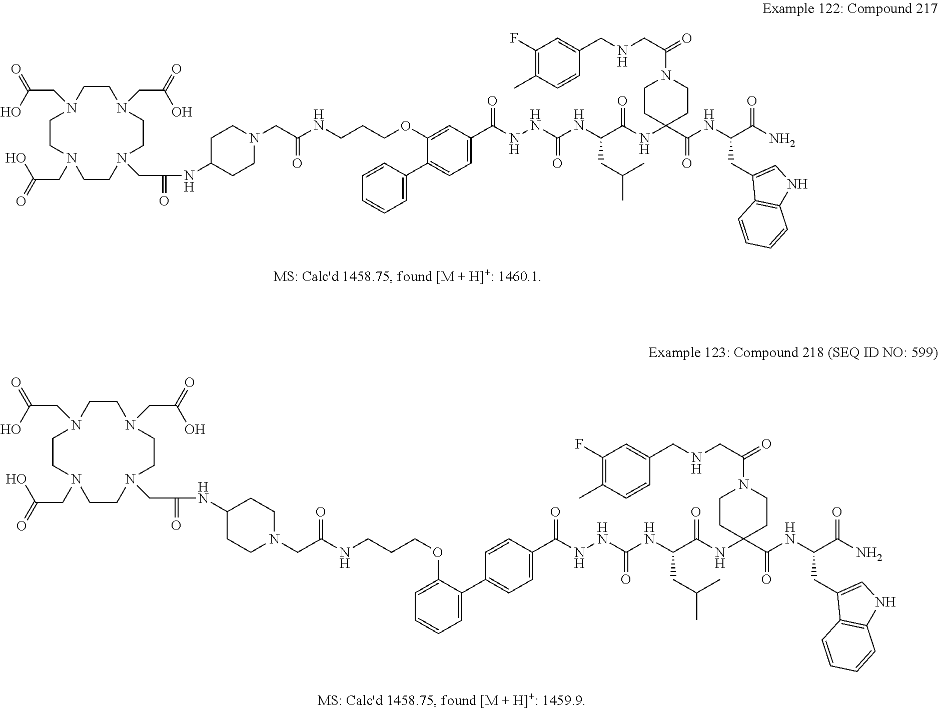 [SMILES: Cc1ccc(CNCC(=O)N2CCC(NC(=O)[C@H](CC(C)C)NC(=O)NNC(=O)c3ccc(-c4ccccc4)c(OCCCNC(=O)CN4CCC(NC(=O)CN5CCN(CC(=O)O)CCN(CC(=O)O)CCN(CC(=O)O)CC5)CC4)c3)(C(=O)N[C@@H](Cc3c[nH]c4ccccc34)C(N)=O)CC2)cc1F ; Cc1ccc(CNCC(=O)N2CCC(NC(=O)[C@H](CC(C)C)NC(=O)NNC(=O)c3ccc(-c4ccccc4OCCCNC(=O)CN4CCC(NC(=O)CN5CCN(CC(=O)O)CCN(CC(=O)O)CCN(CC(=O)O)CC5)CC4)cc3)(C(=O)N[C@@H](Cc3c[nH]c4ccccc34)C(N)=O)CC2)cc1F]

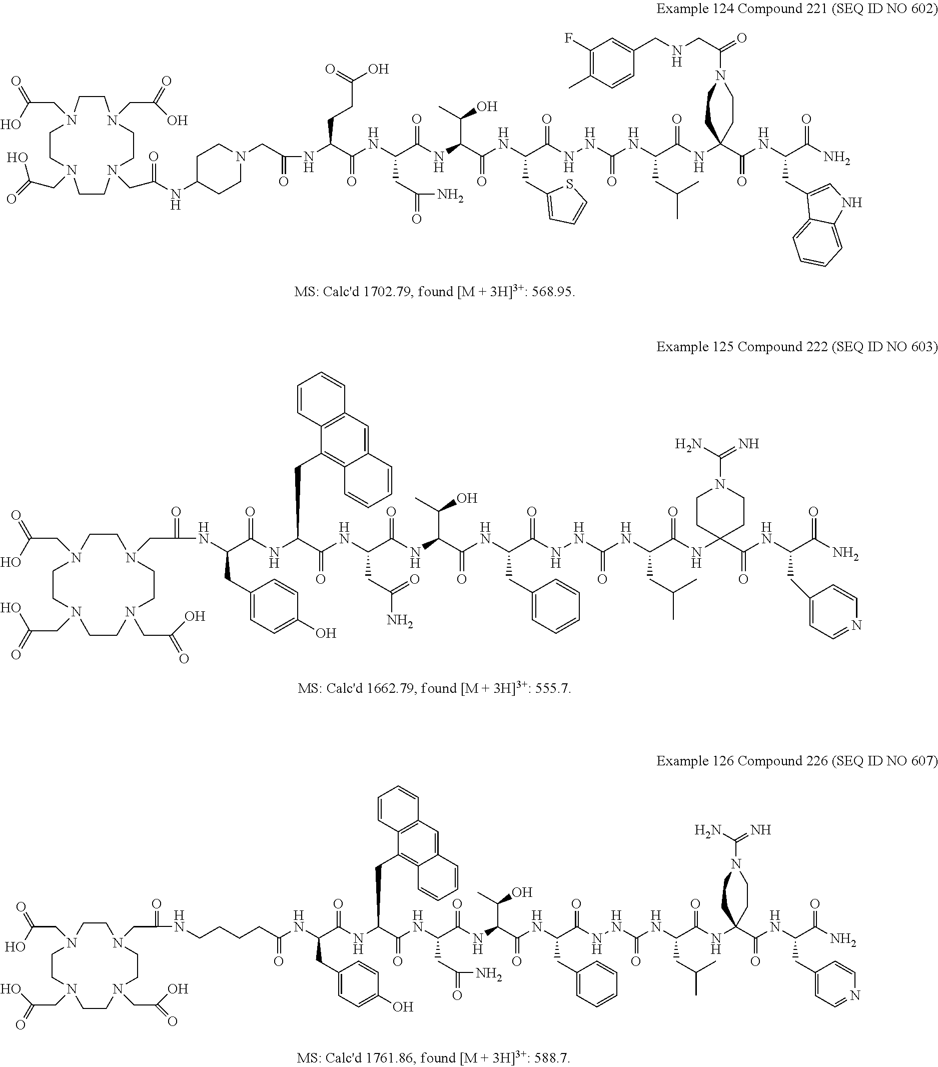

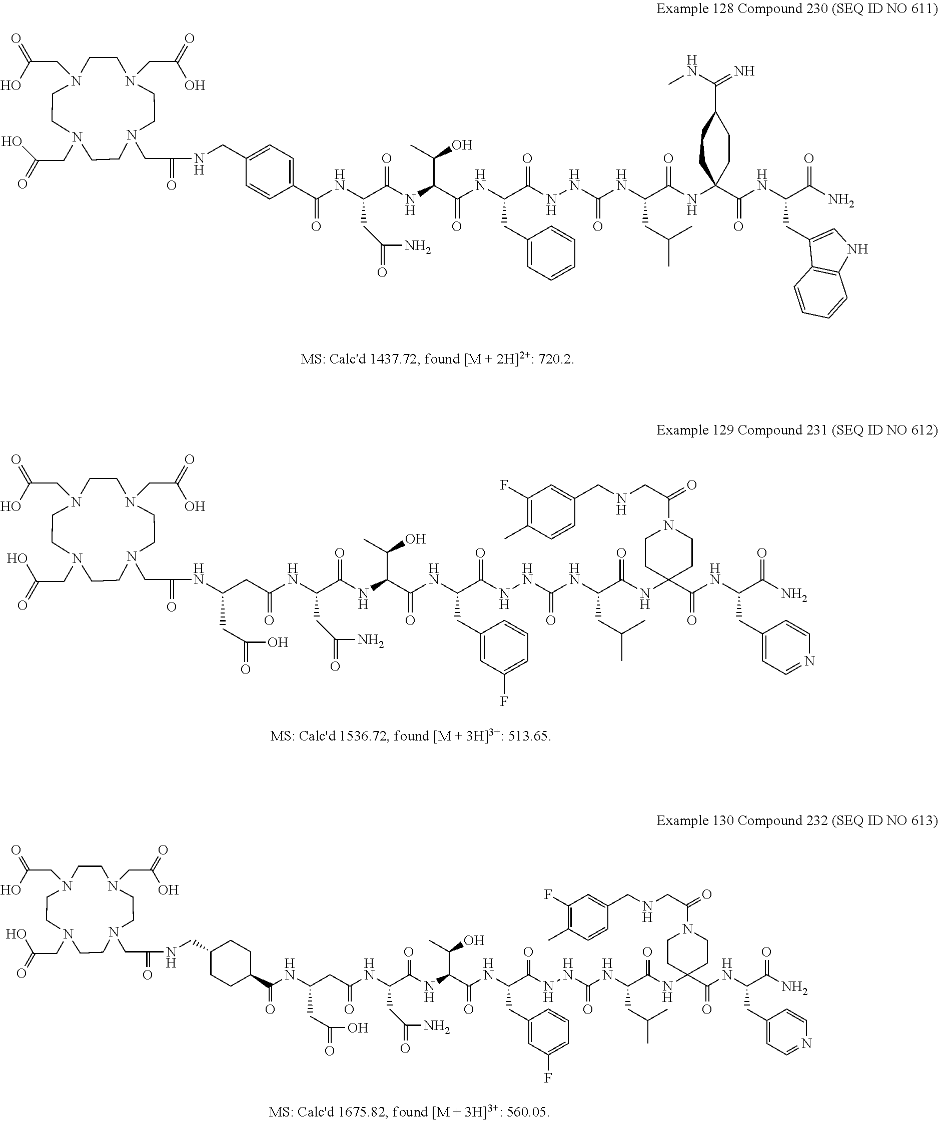

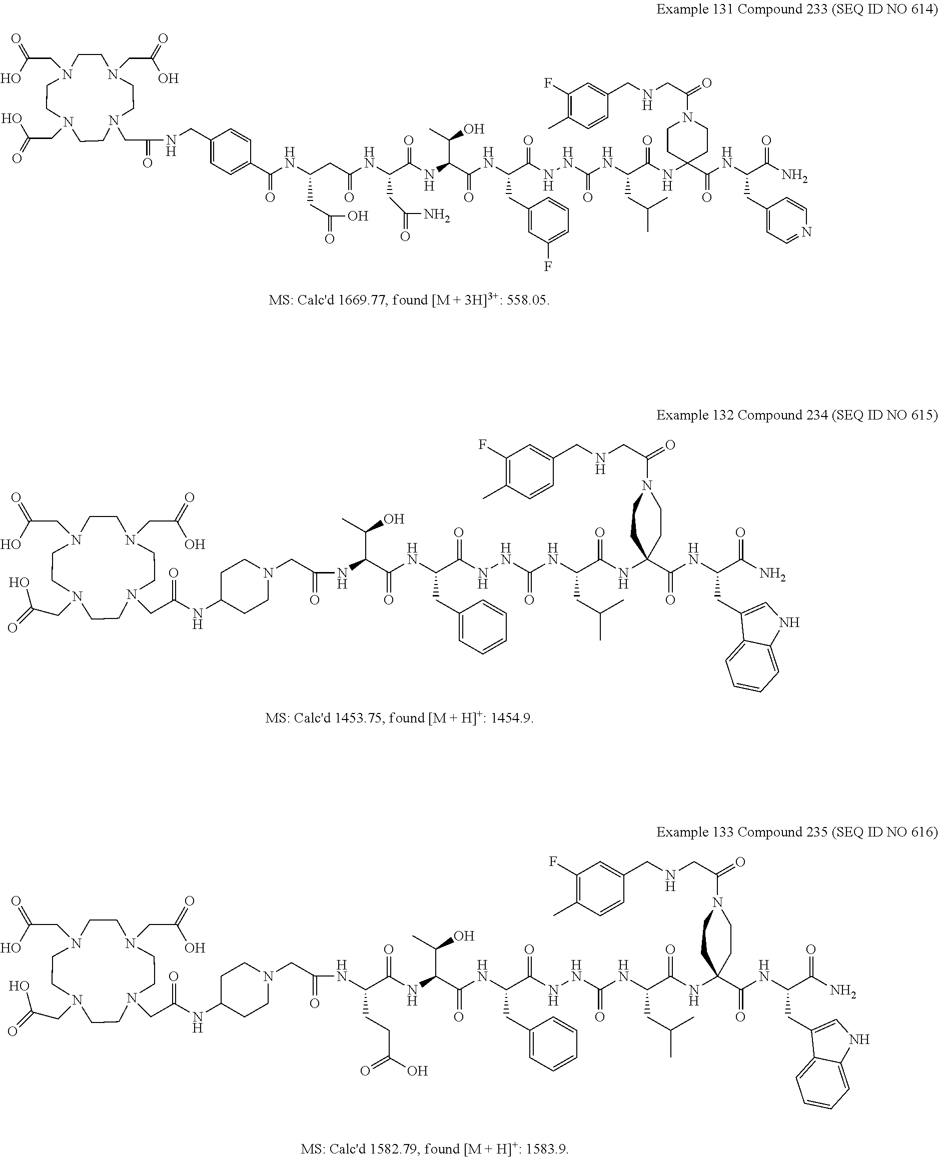

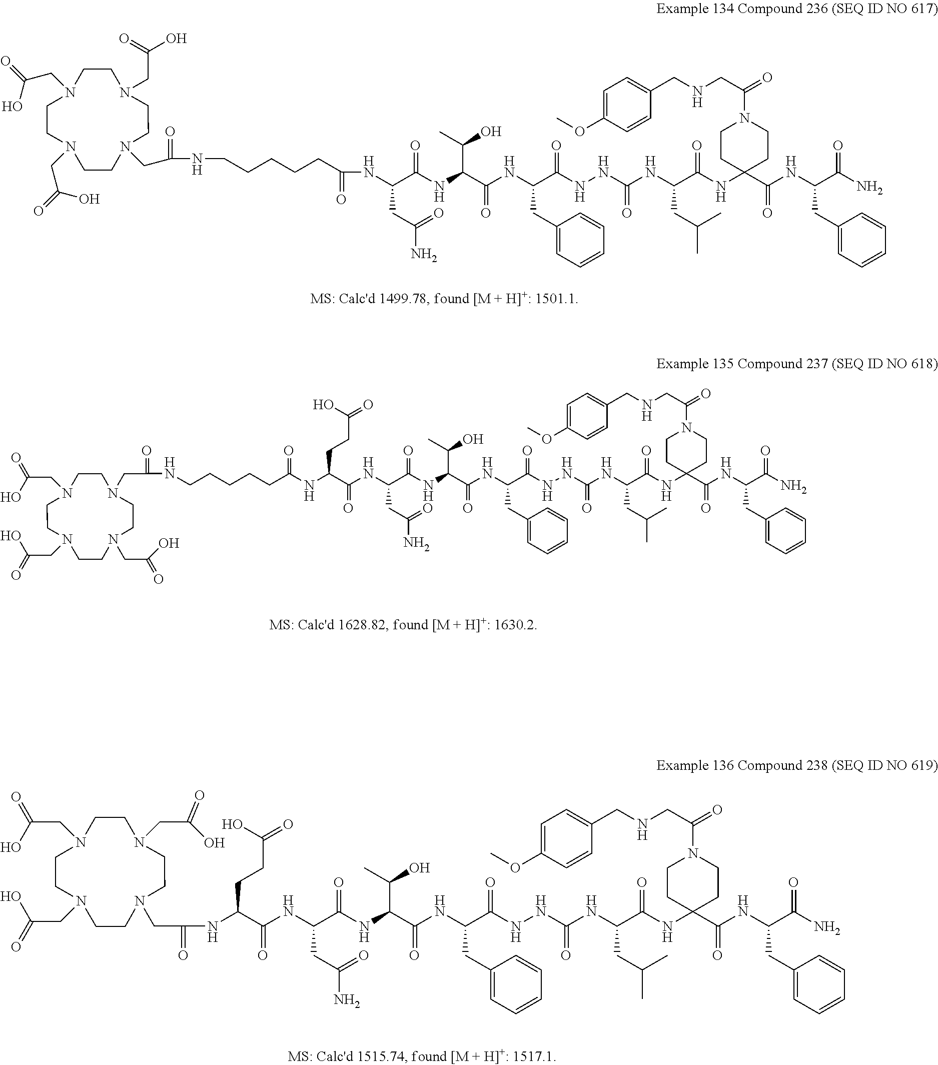

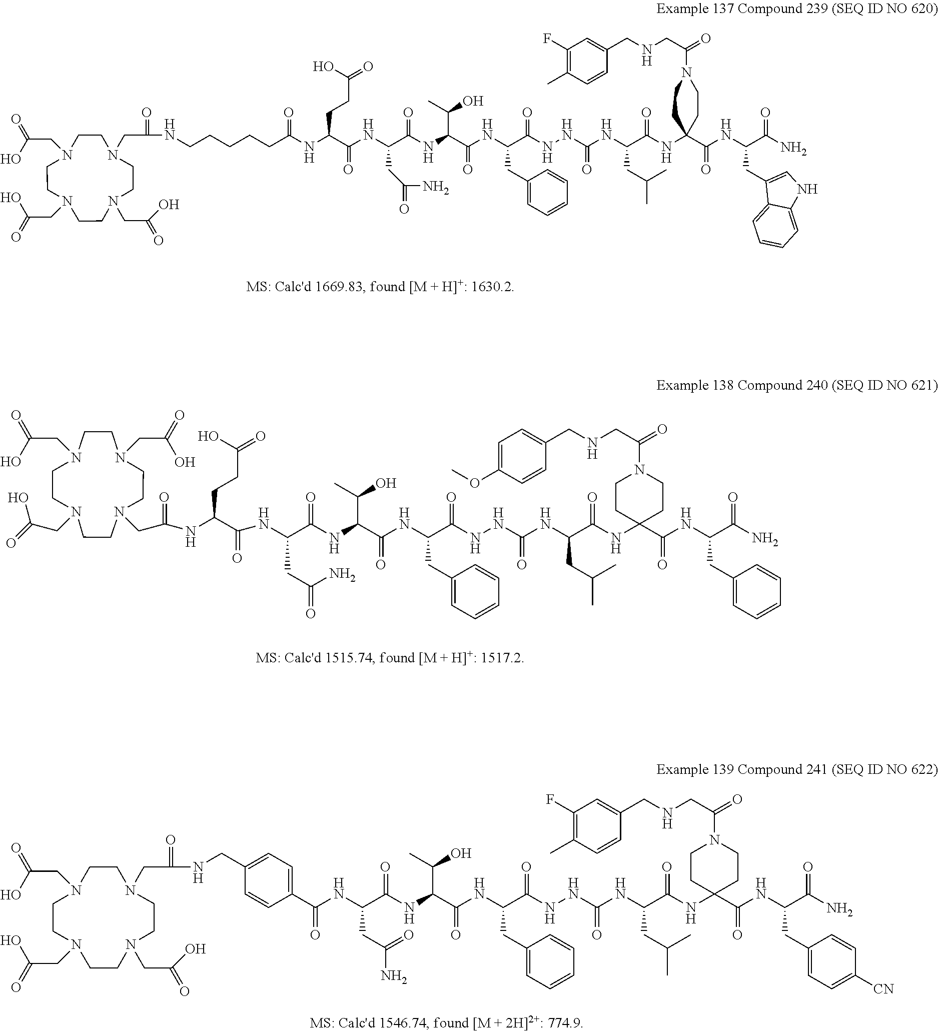

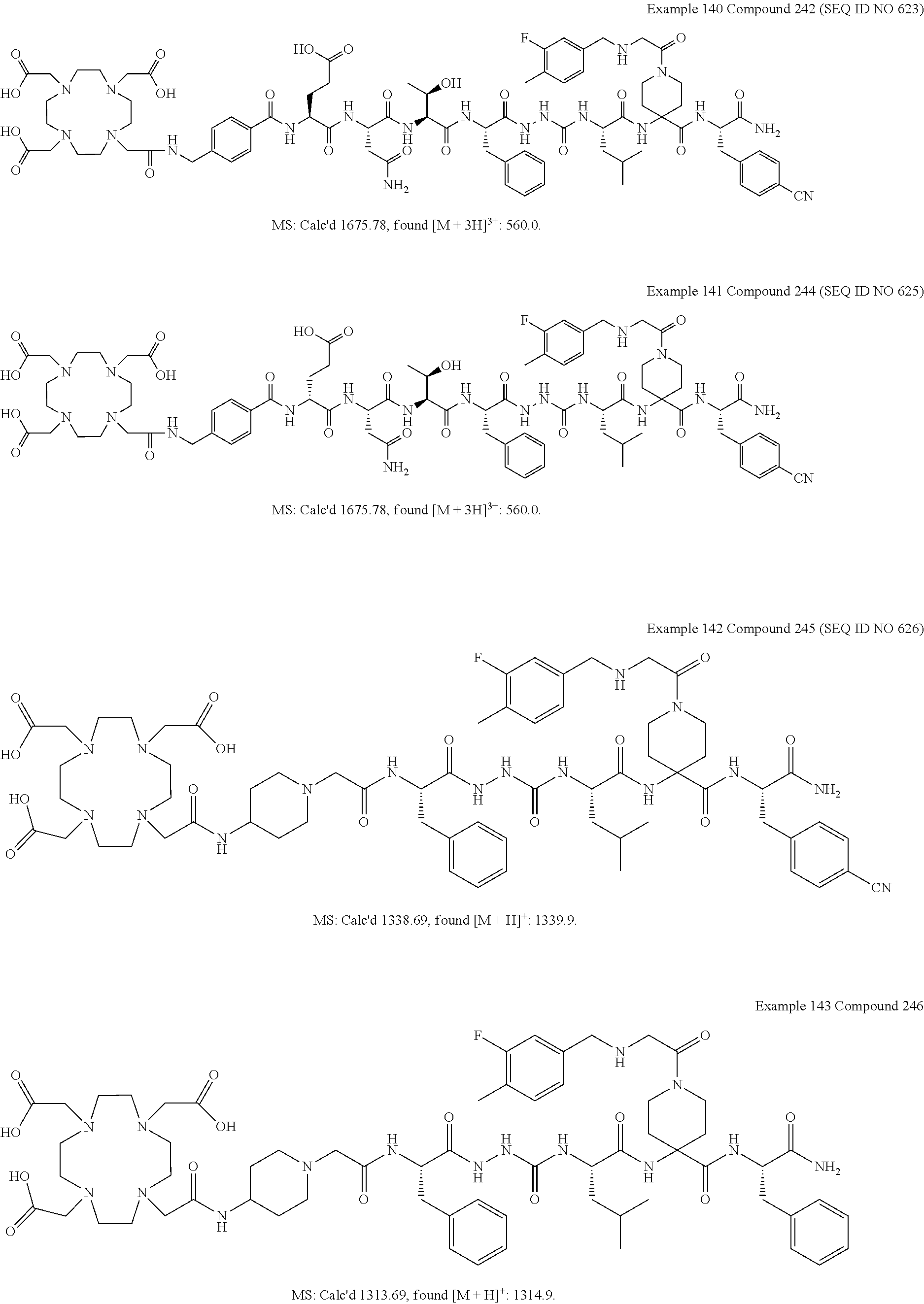

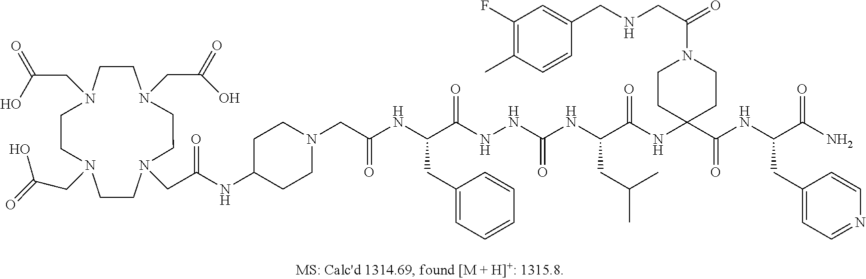 [SMILES: Cc1ccc(CNCC(=O)N2CCC(NC(=O)[C@H](CC(C)C)NC(=O)NNC(=O)[C@H](Cc3ccccc3)NC(=O)CN3CCC(NC(=O)CN4CCN(CC(=O)O)CCN(CC(=O)O)CCN(CC(=O)O)CC4)CC3)(C(=O)N[C@@H](Cc3ccncc3)C(N)=O)CC2)cc1F]

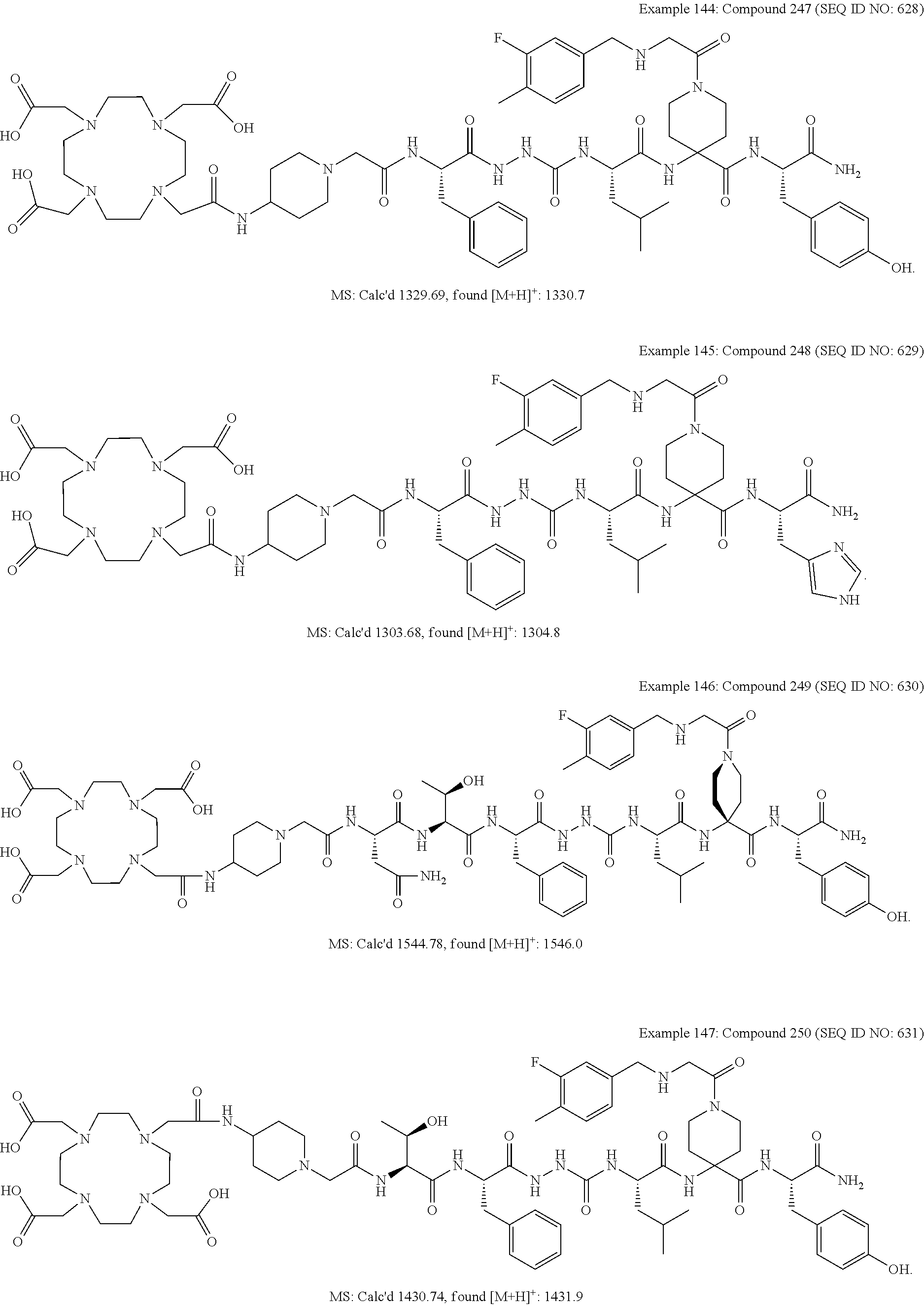

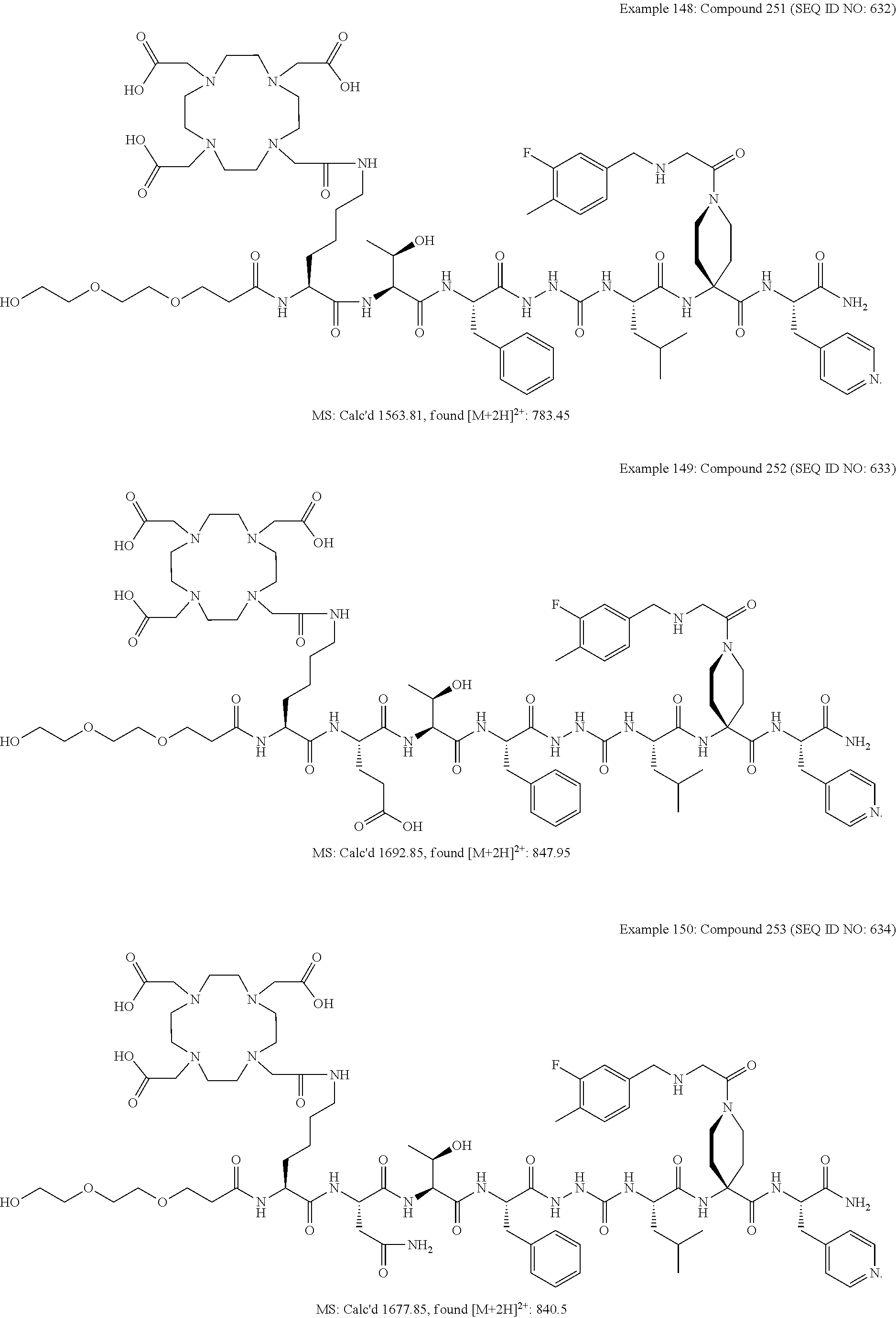

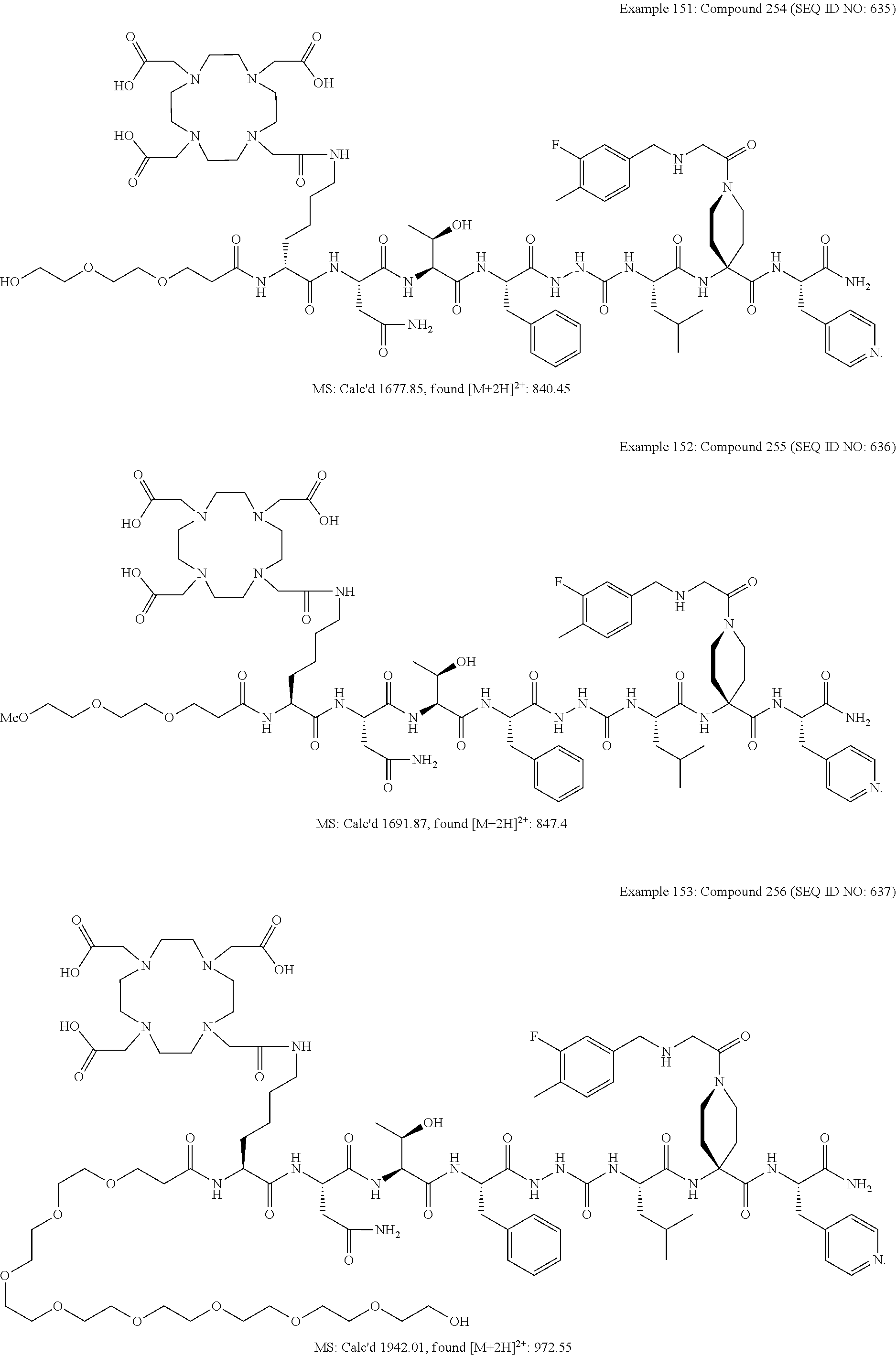

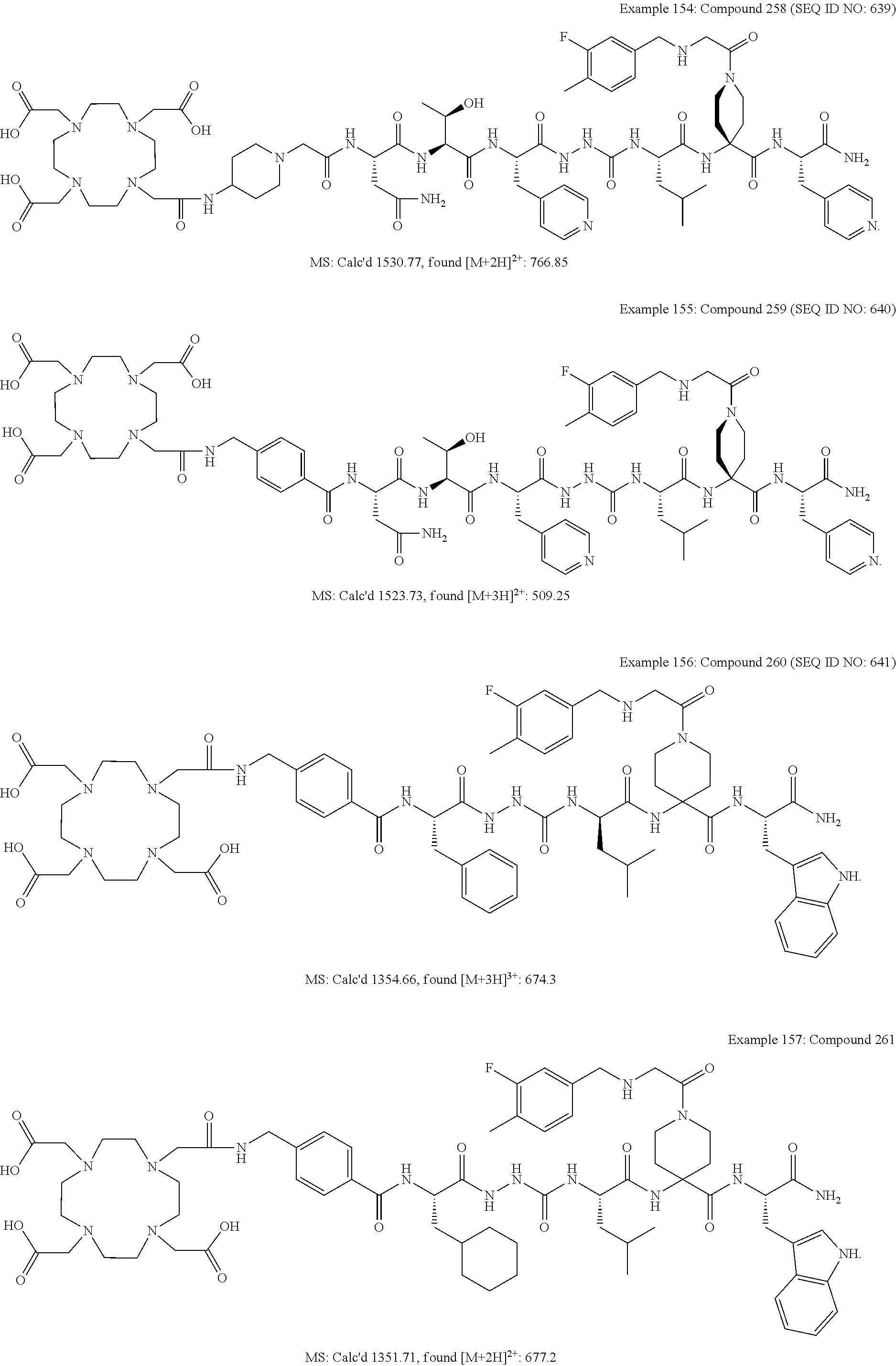

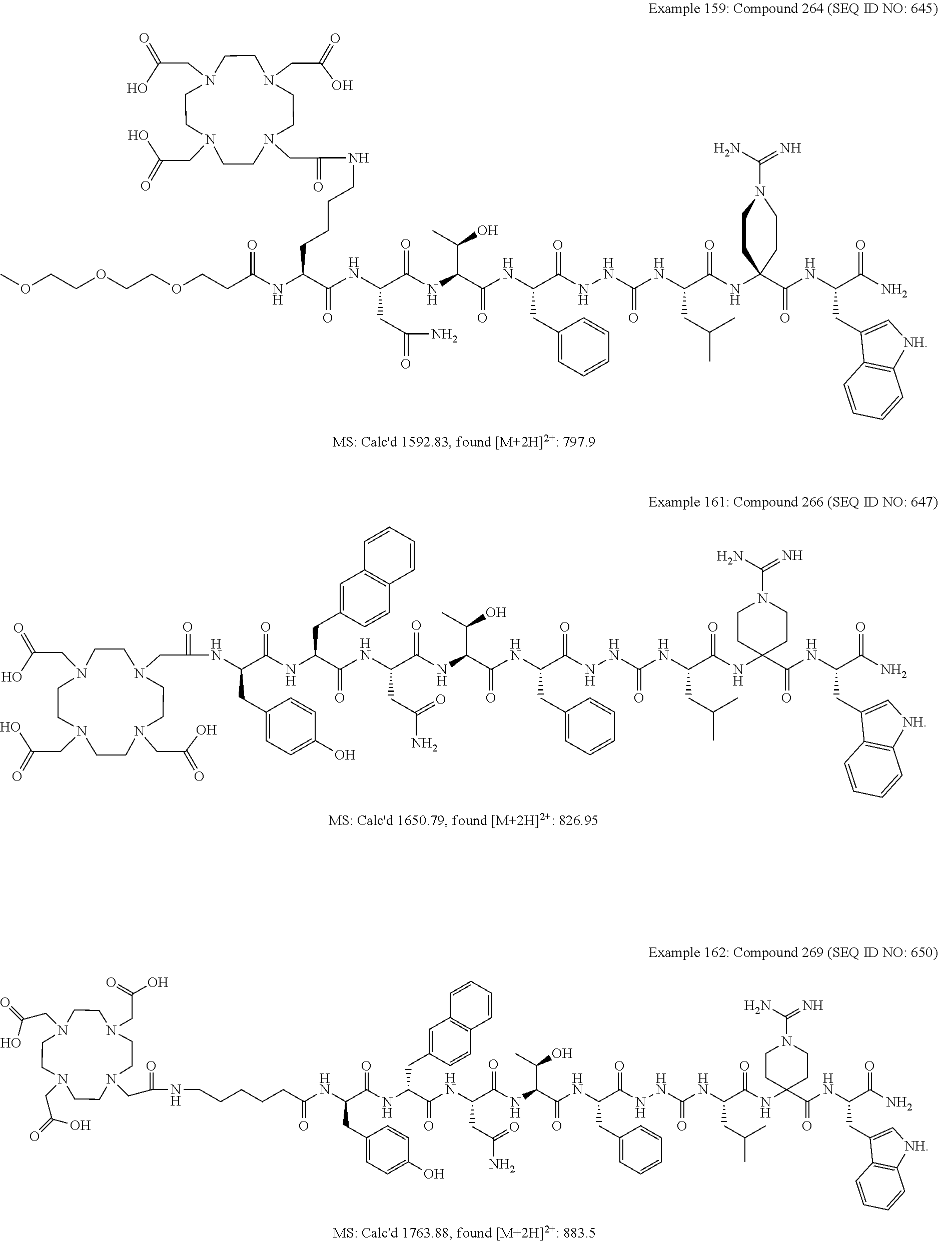

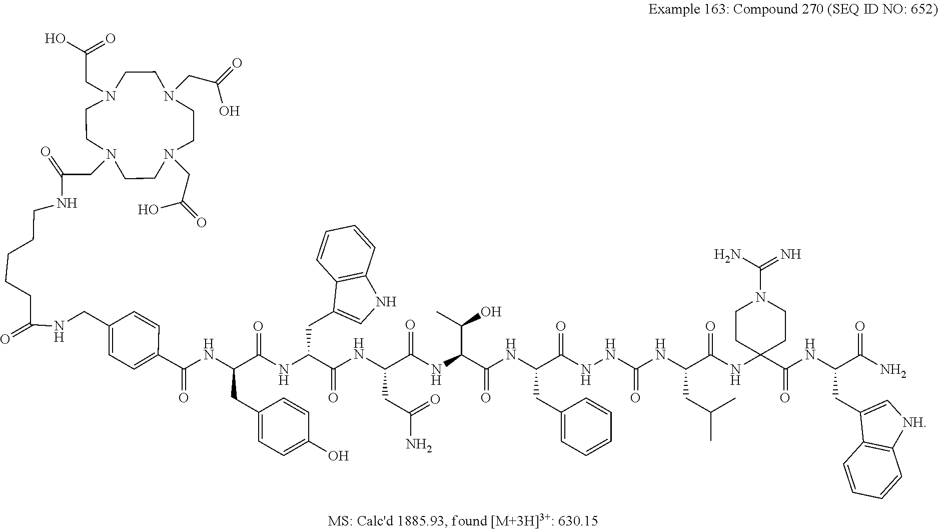 [SMILES: CC(C)C[C@H](NC(=O)NNC(=O)[C@H](Cc1ccccc1)NC(=O)[C@@H](NC(=O)[C@H](CC(N)=O)NC(=O)[C@@H](Cc1c[nH]c2ccccc12)NC(=O)[C@@H](Cc1ccc(O)cc1)NC(=O)c1ccc(CNC(=O)CCCCCNC(=O)CN2CCN(CC(=O)O)CCN(CC(=O)O)CCN(CC(=O)O)CC2)cc1)[C@@H](C)O)C(=O)NC1(C(=O)N[C@@H](Cc2c[nH]c3ccccc23)C(N)=O)CCN(C(=N)N)CC1]

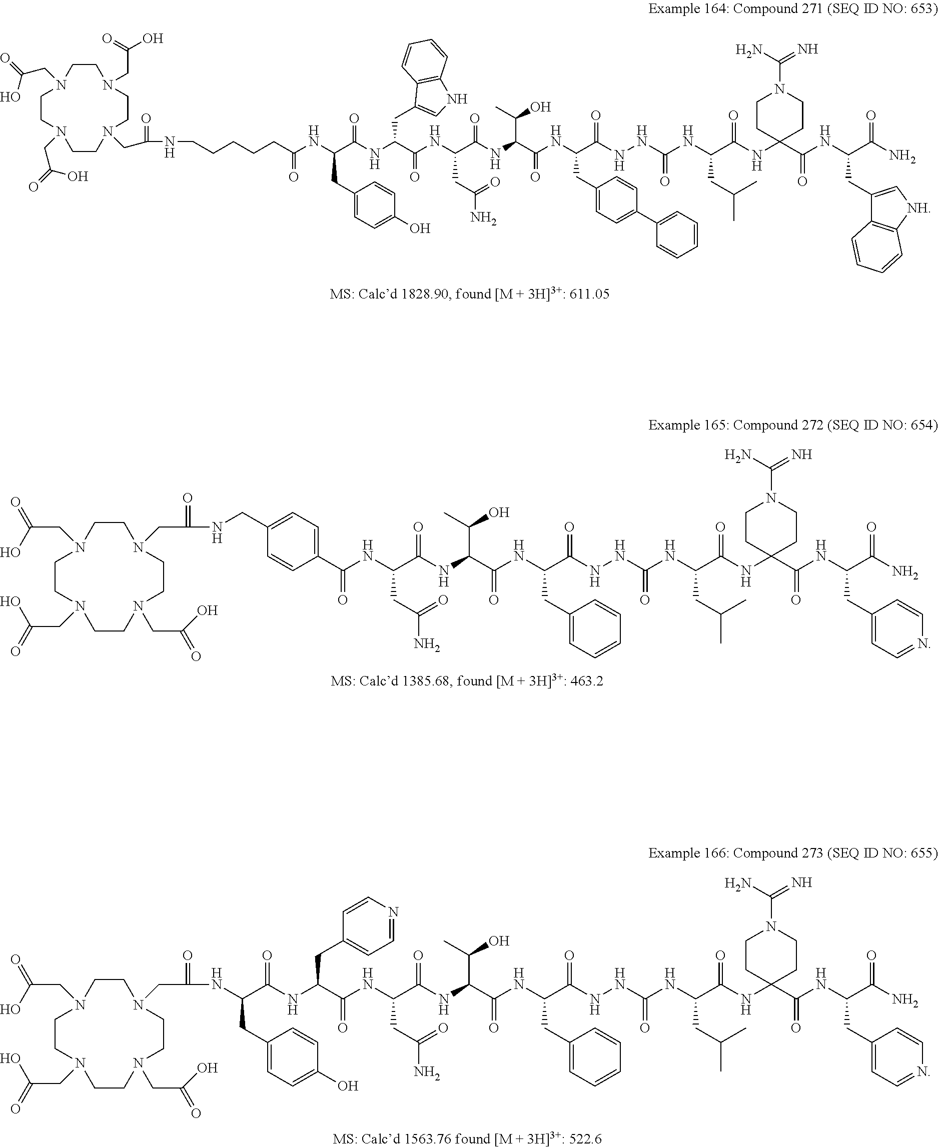

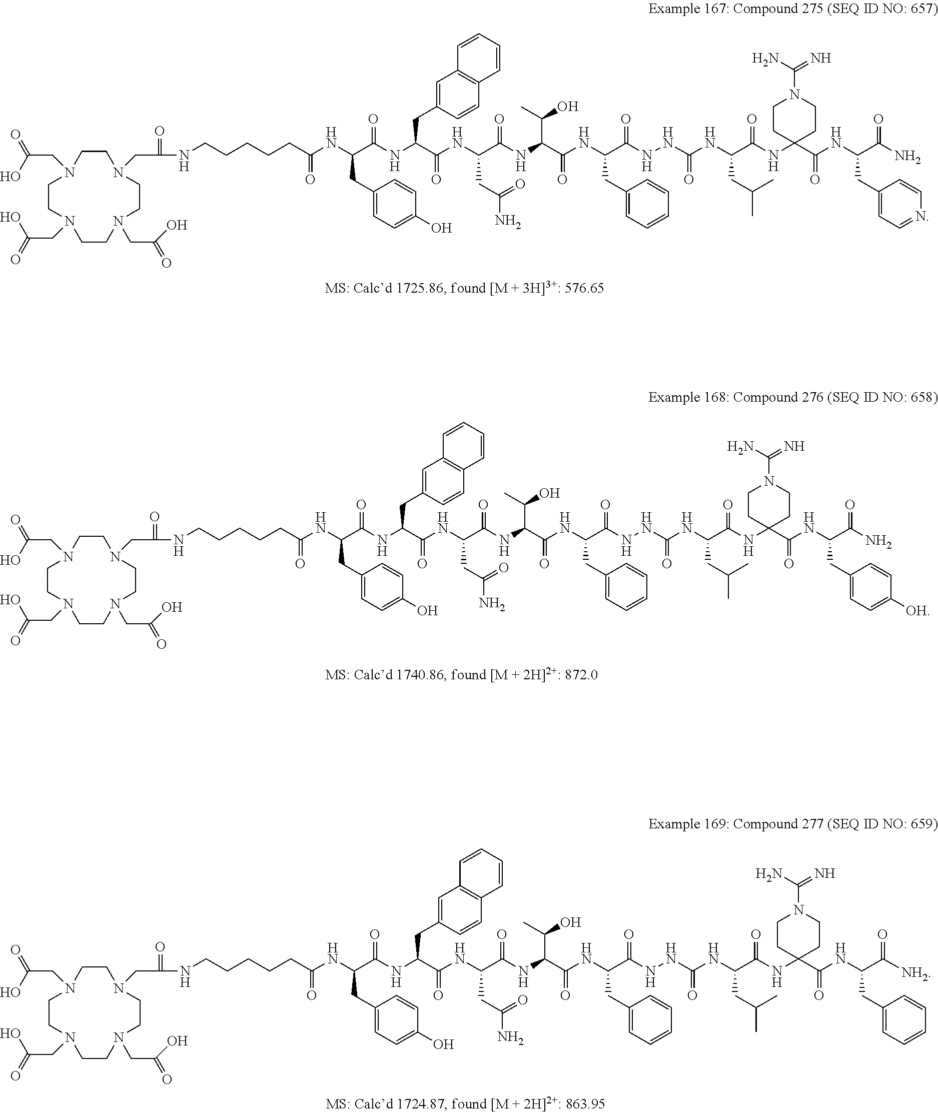

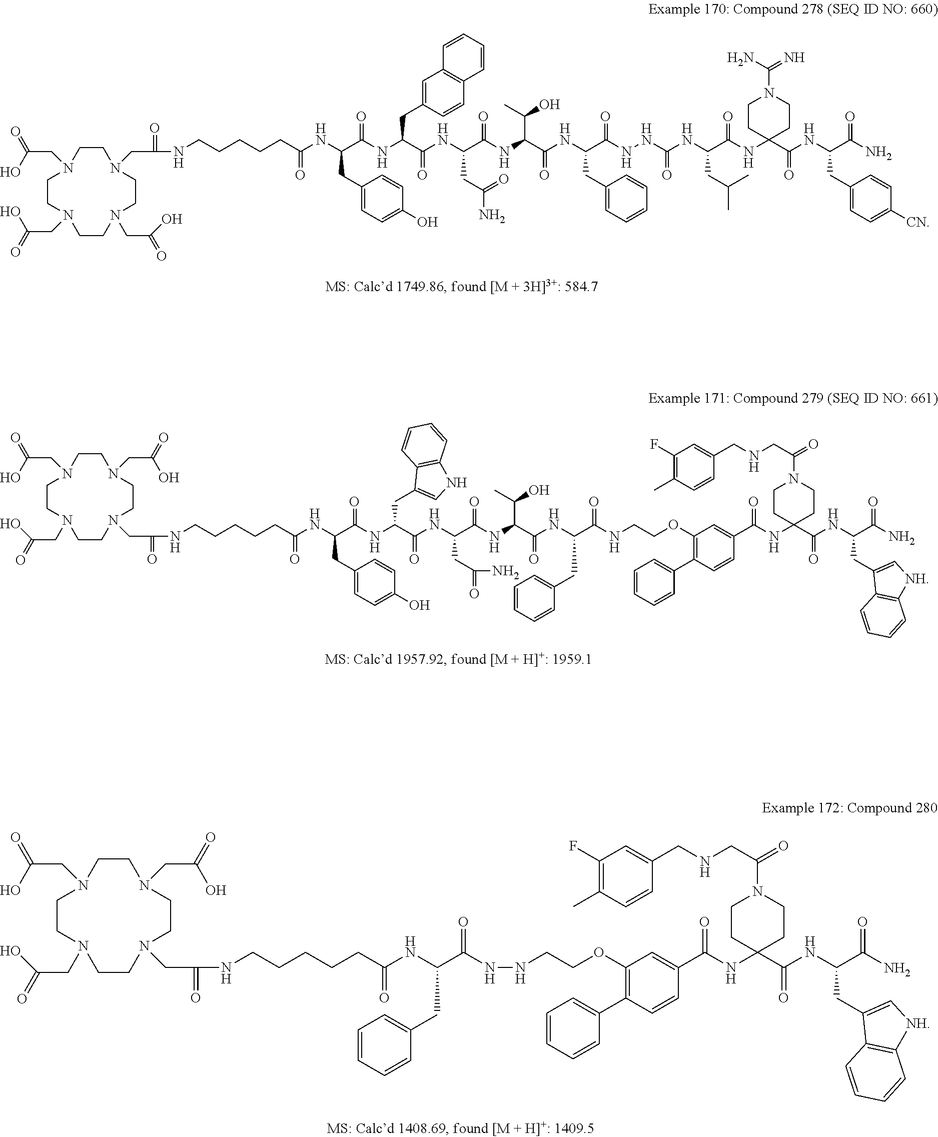

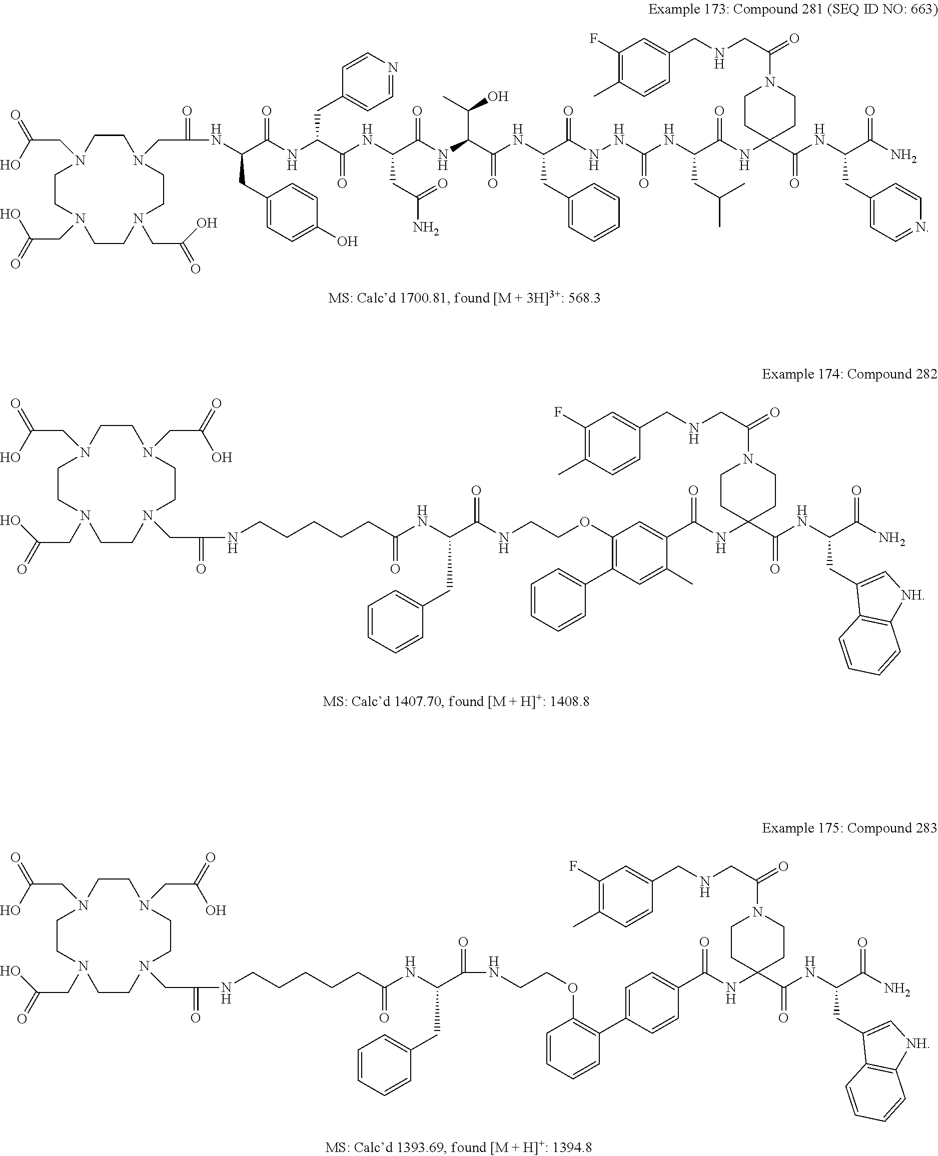

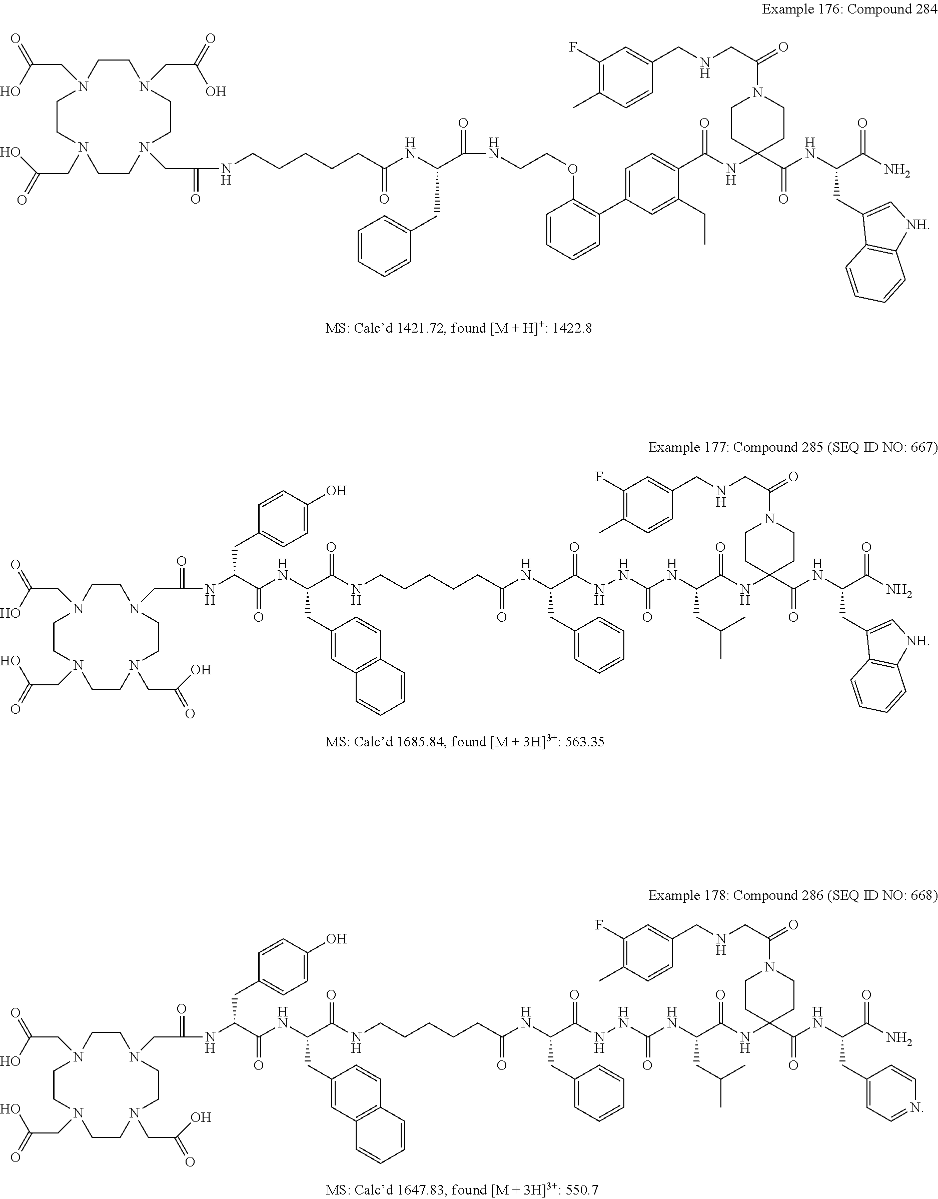

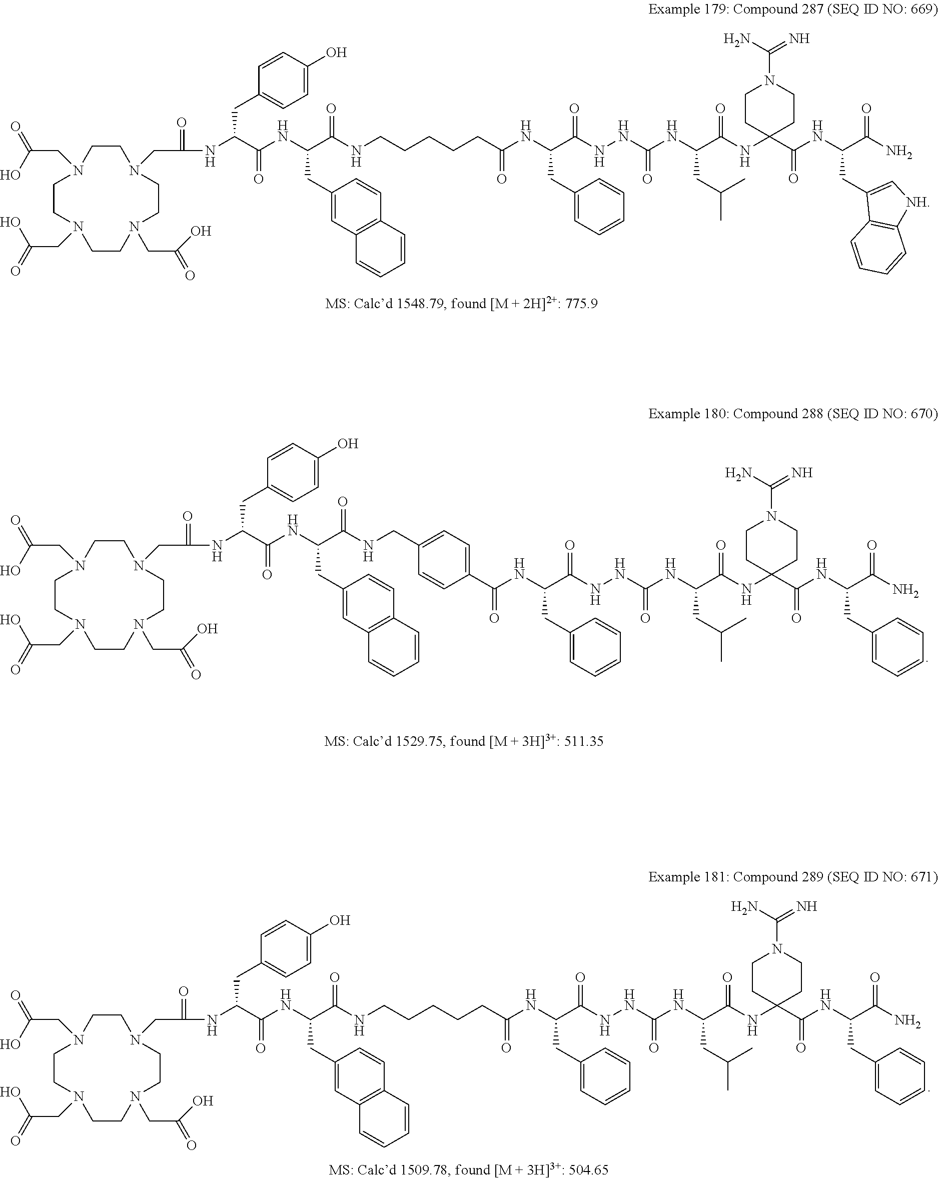

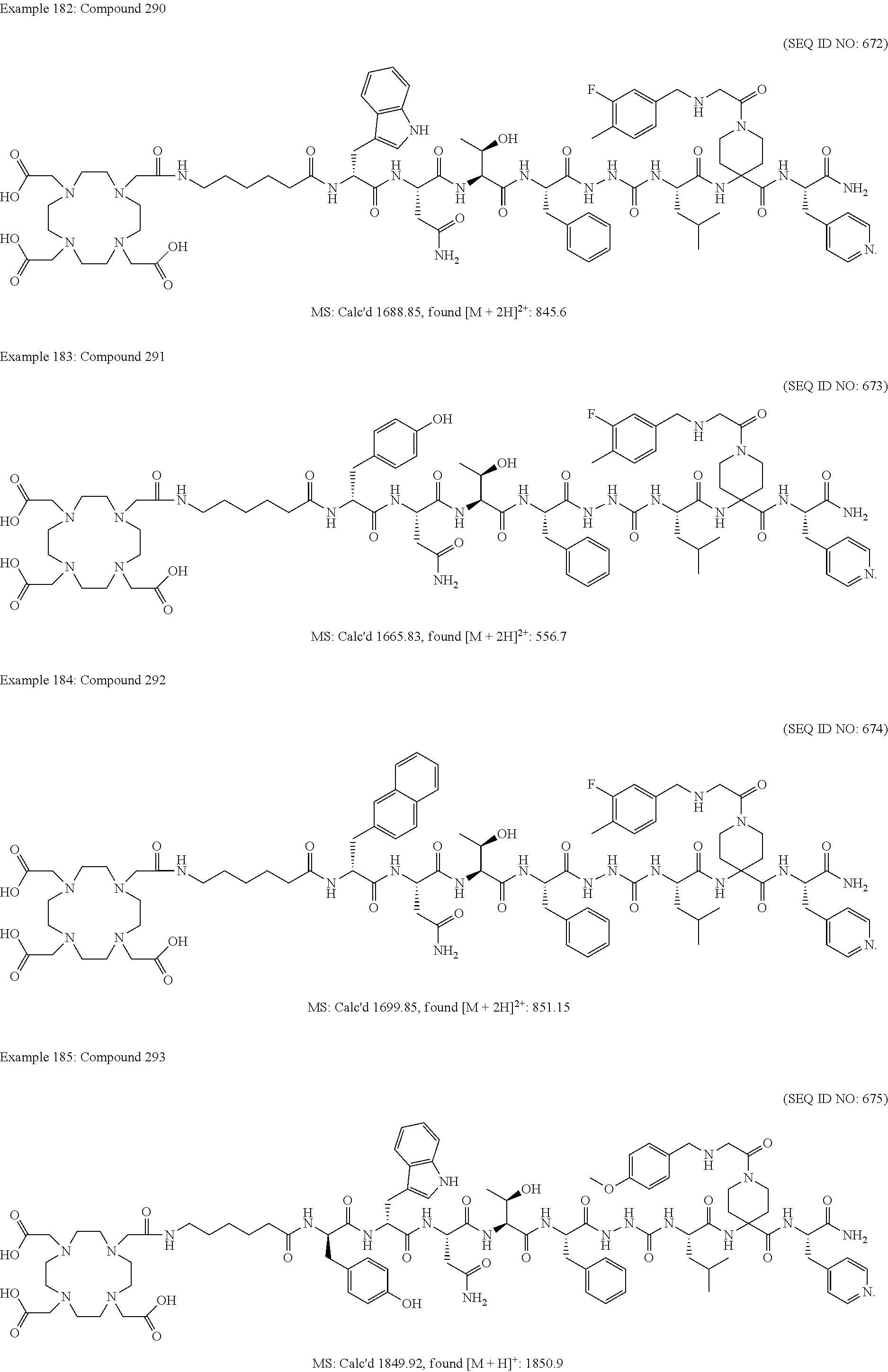

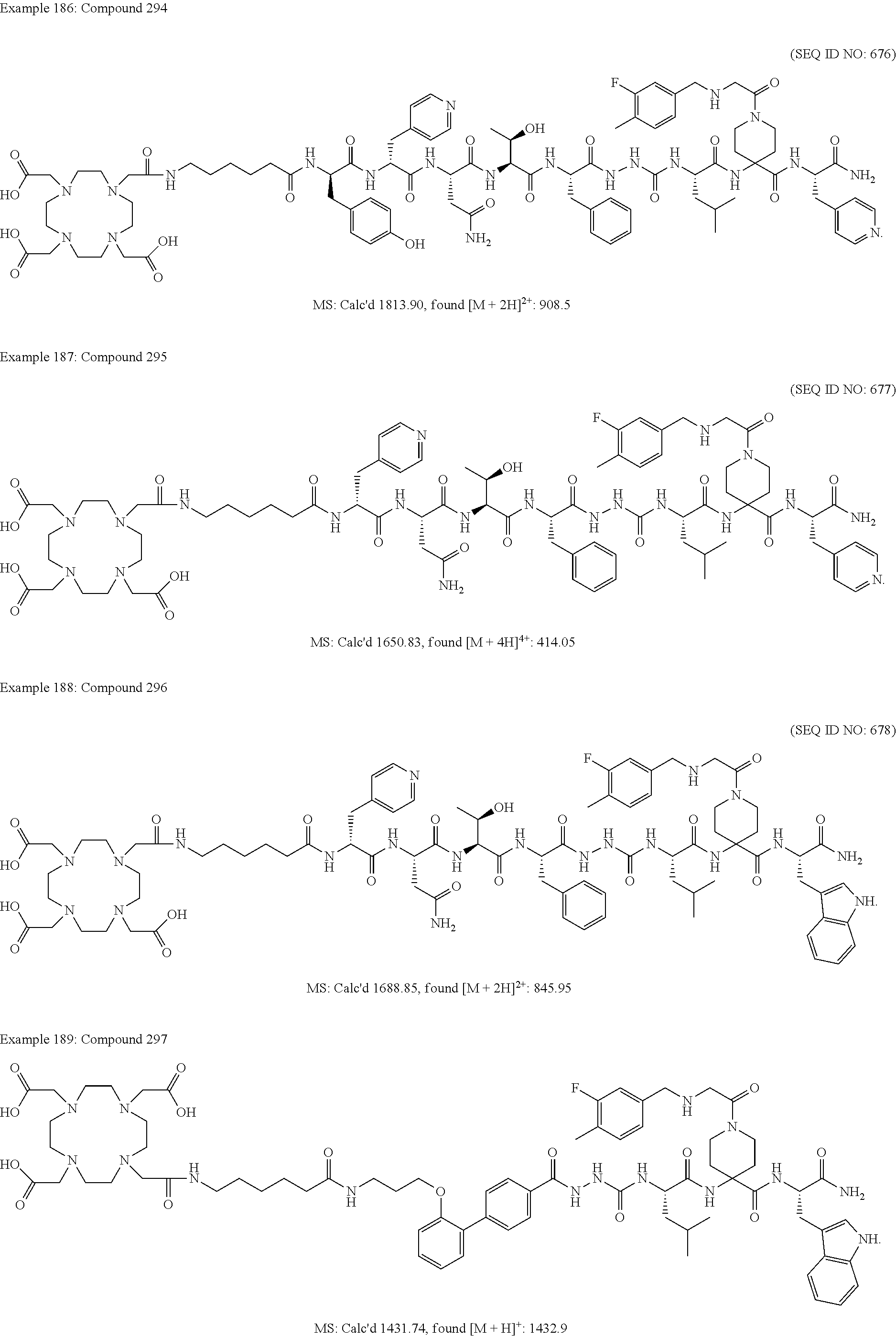

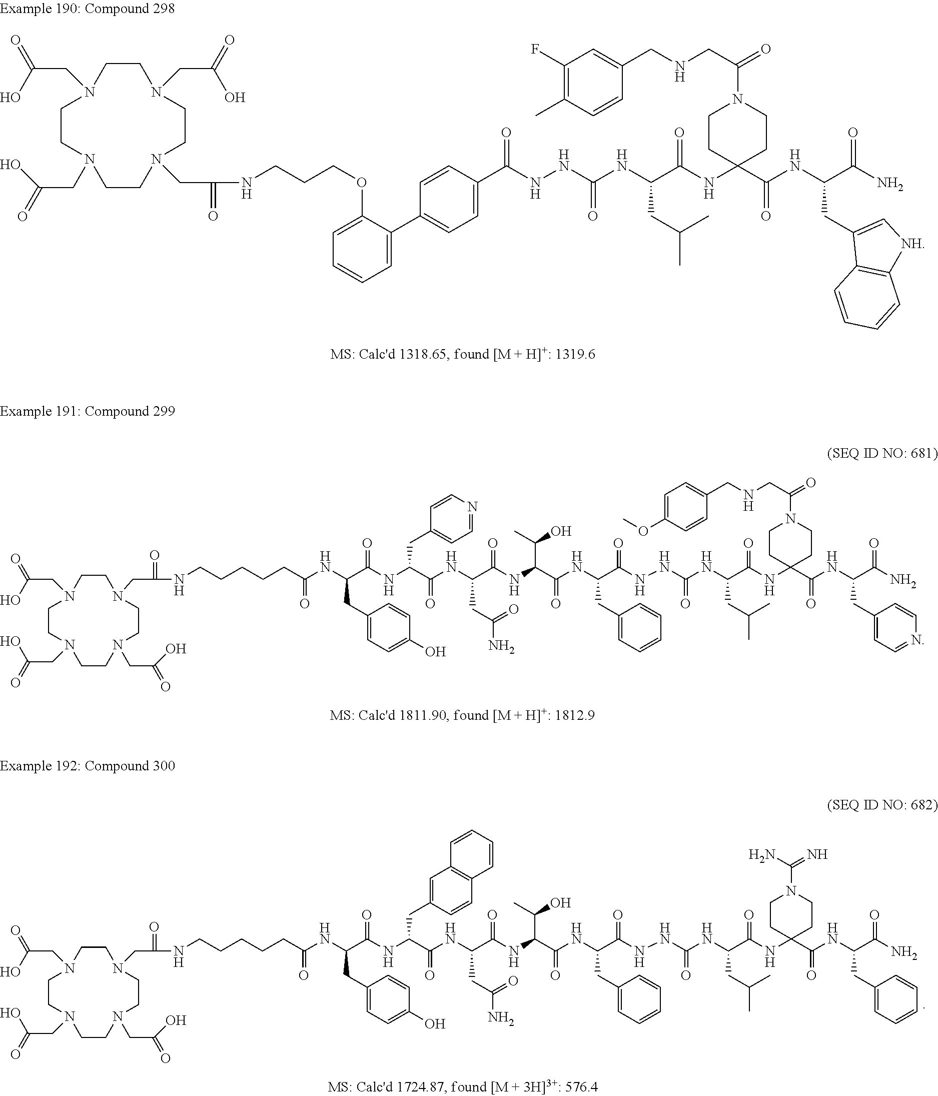

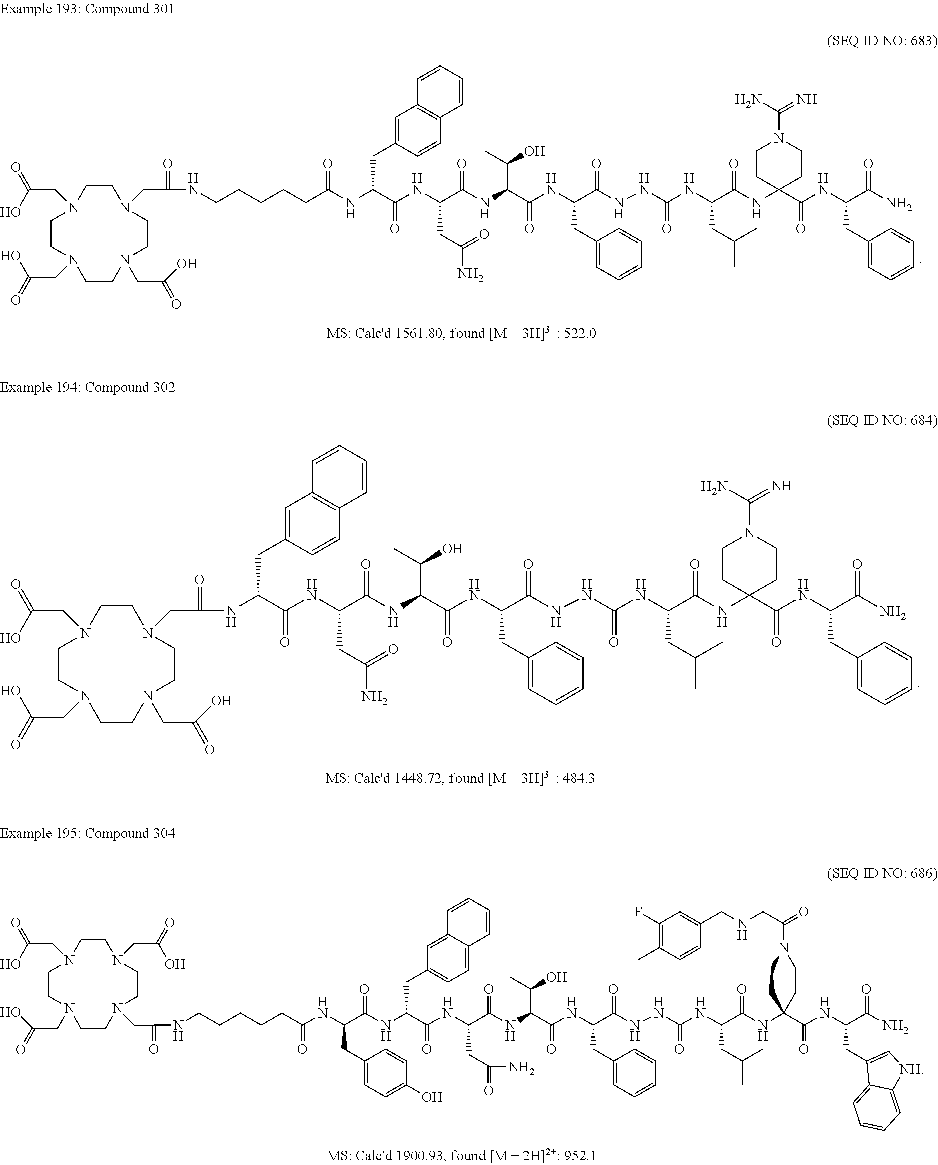

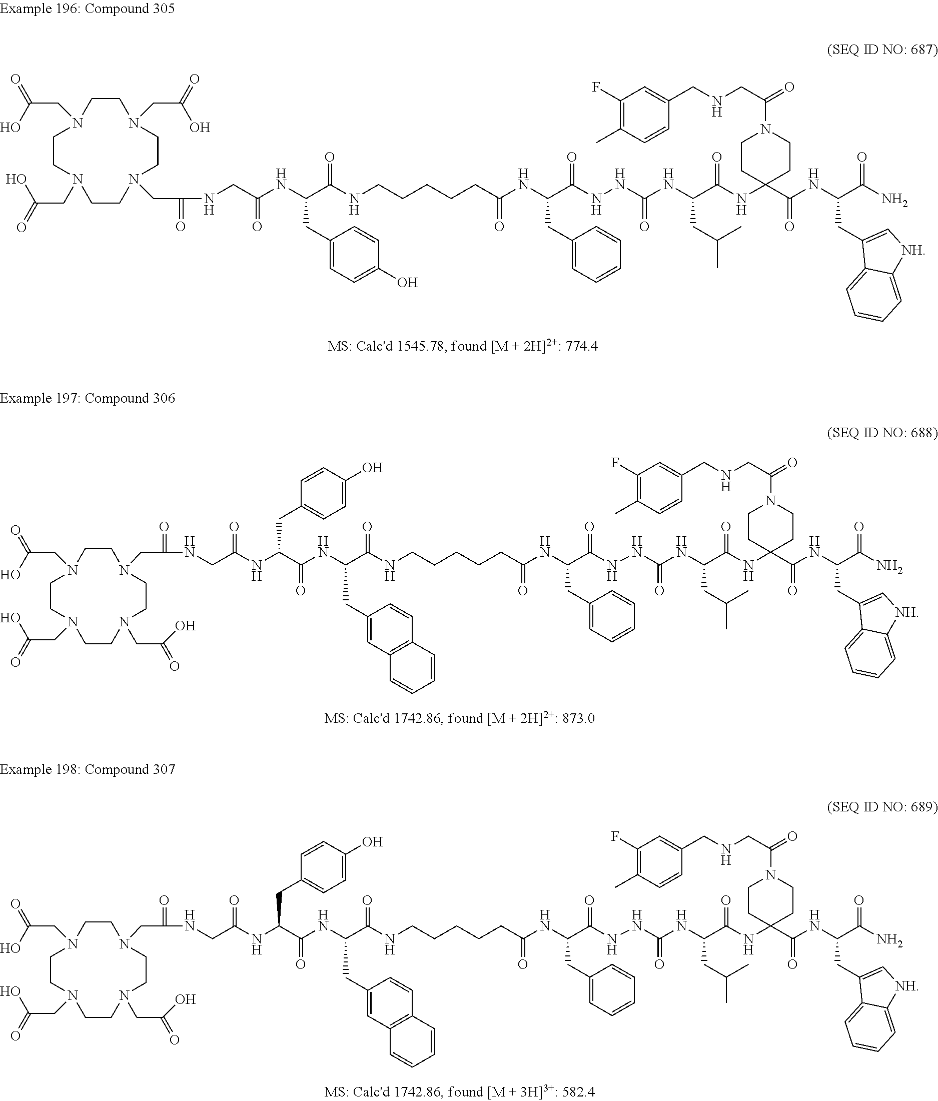

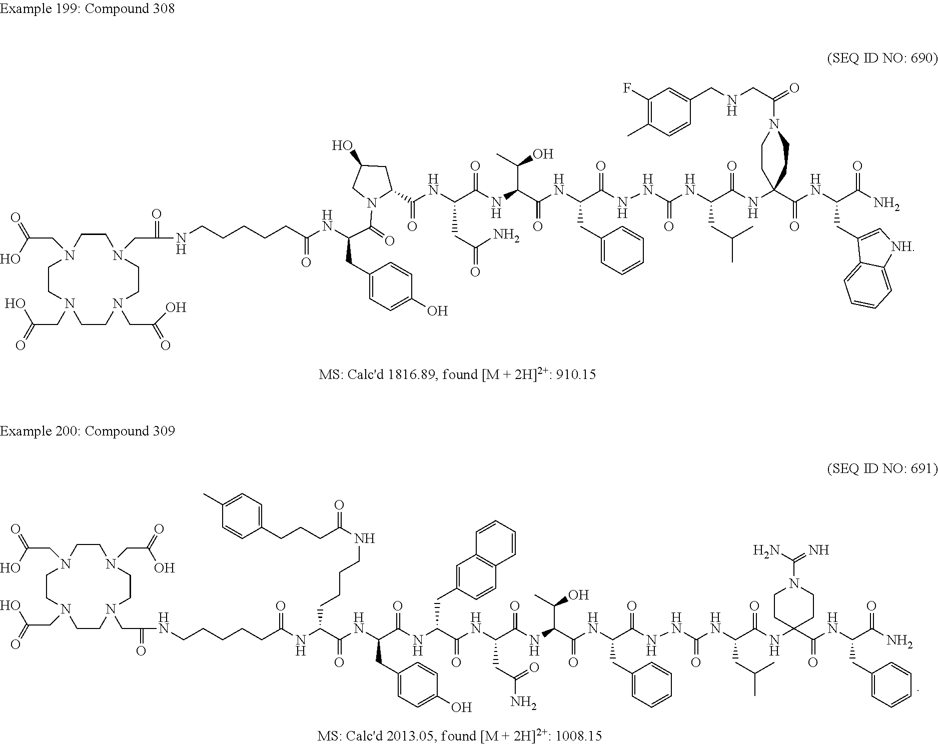

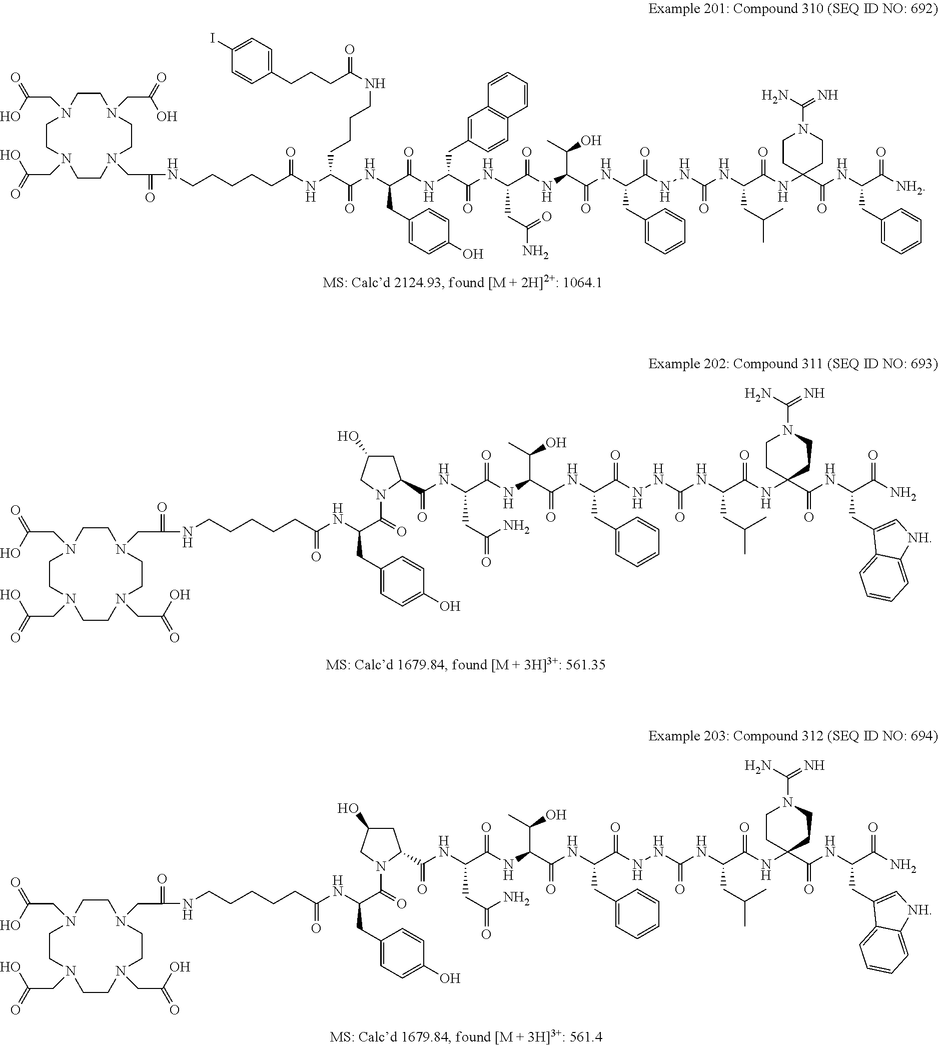

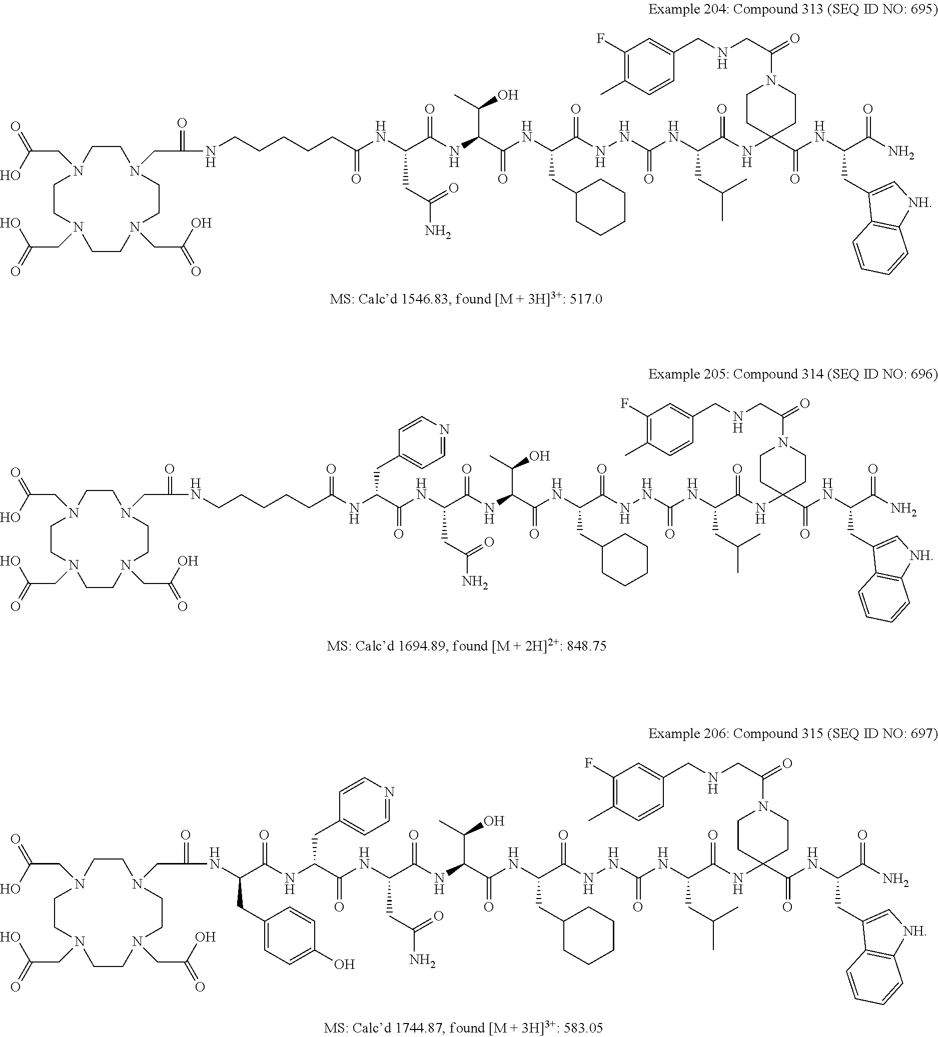

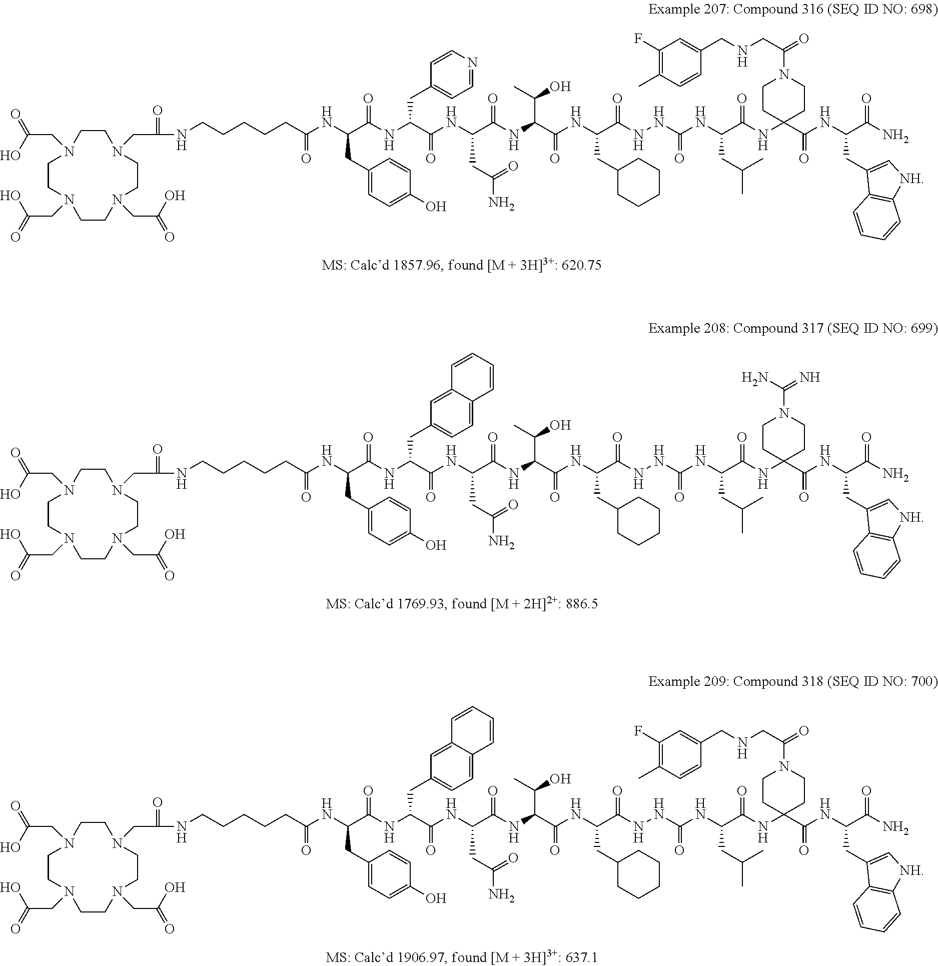

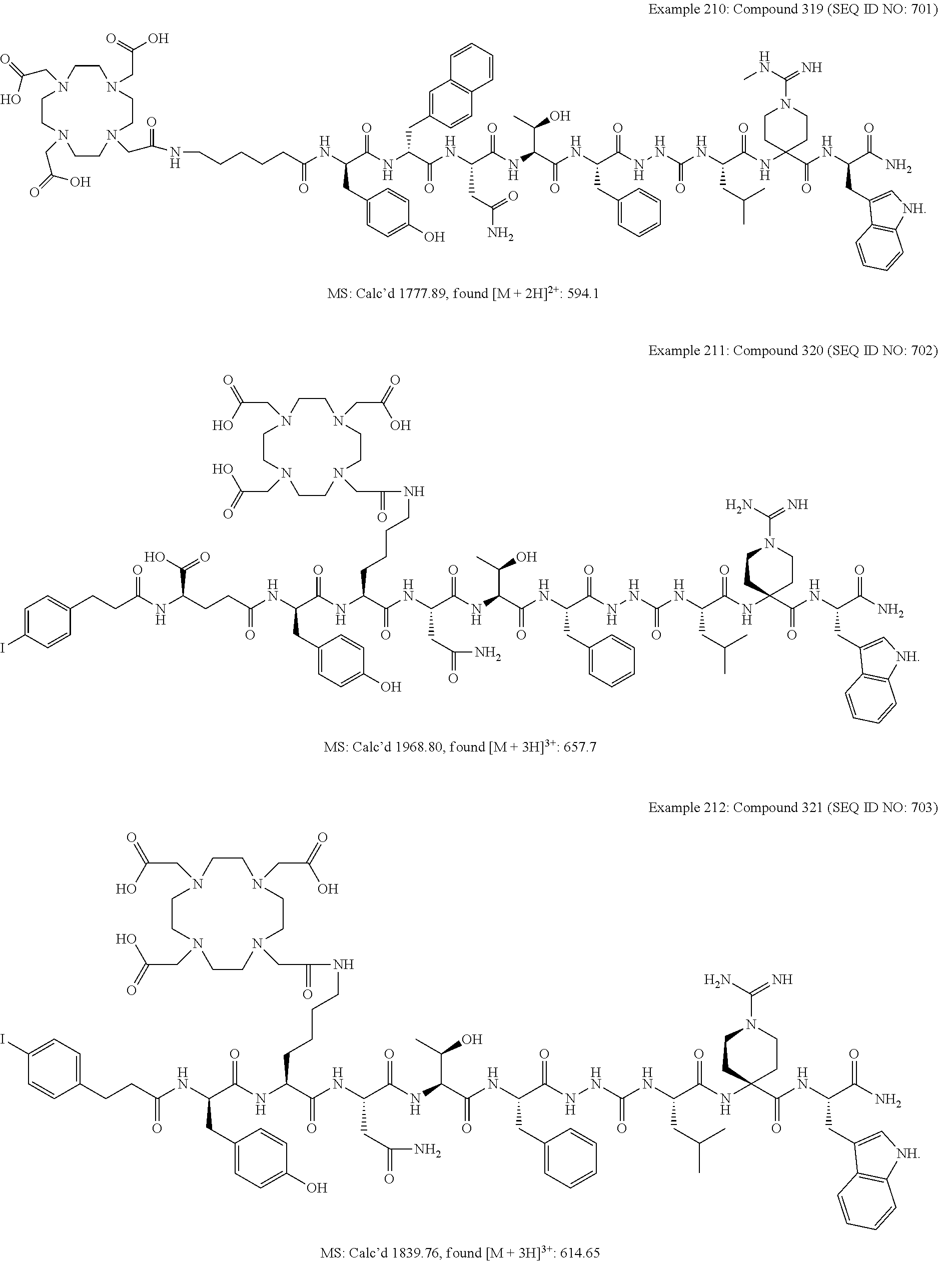

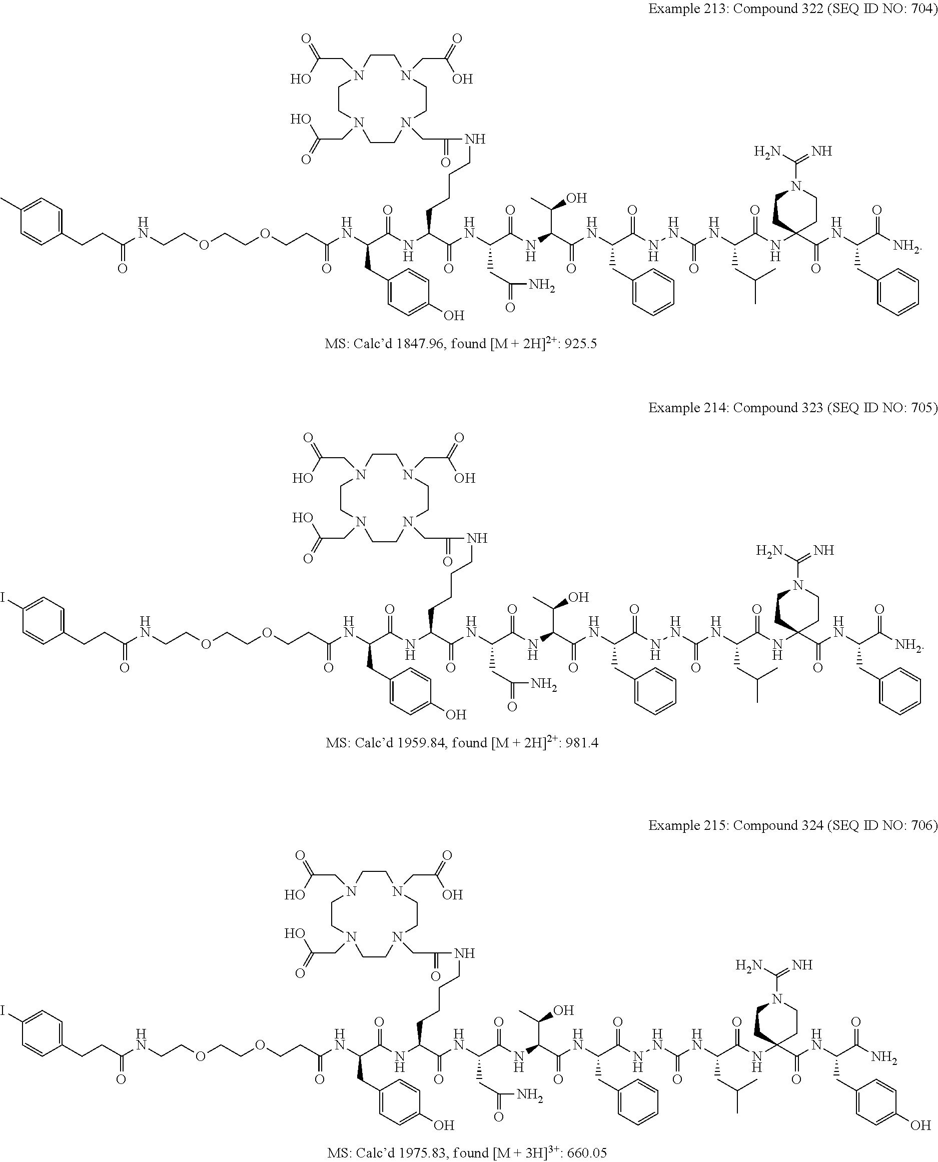

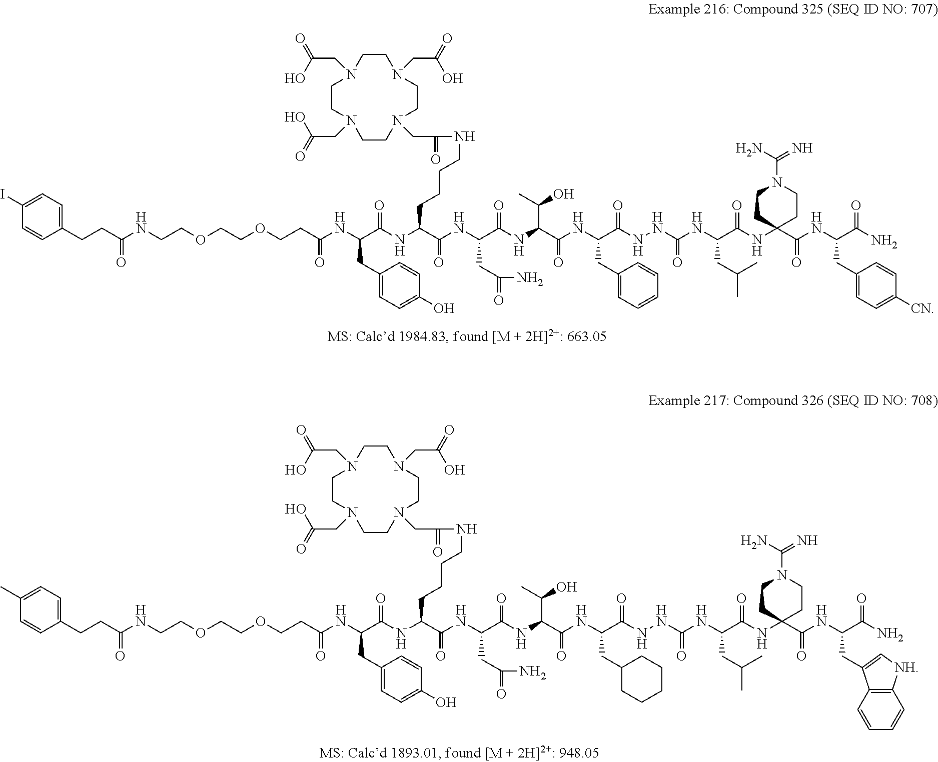

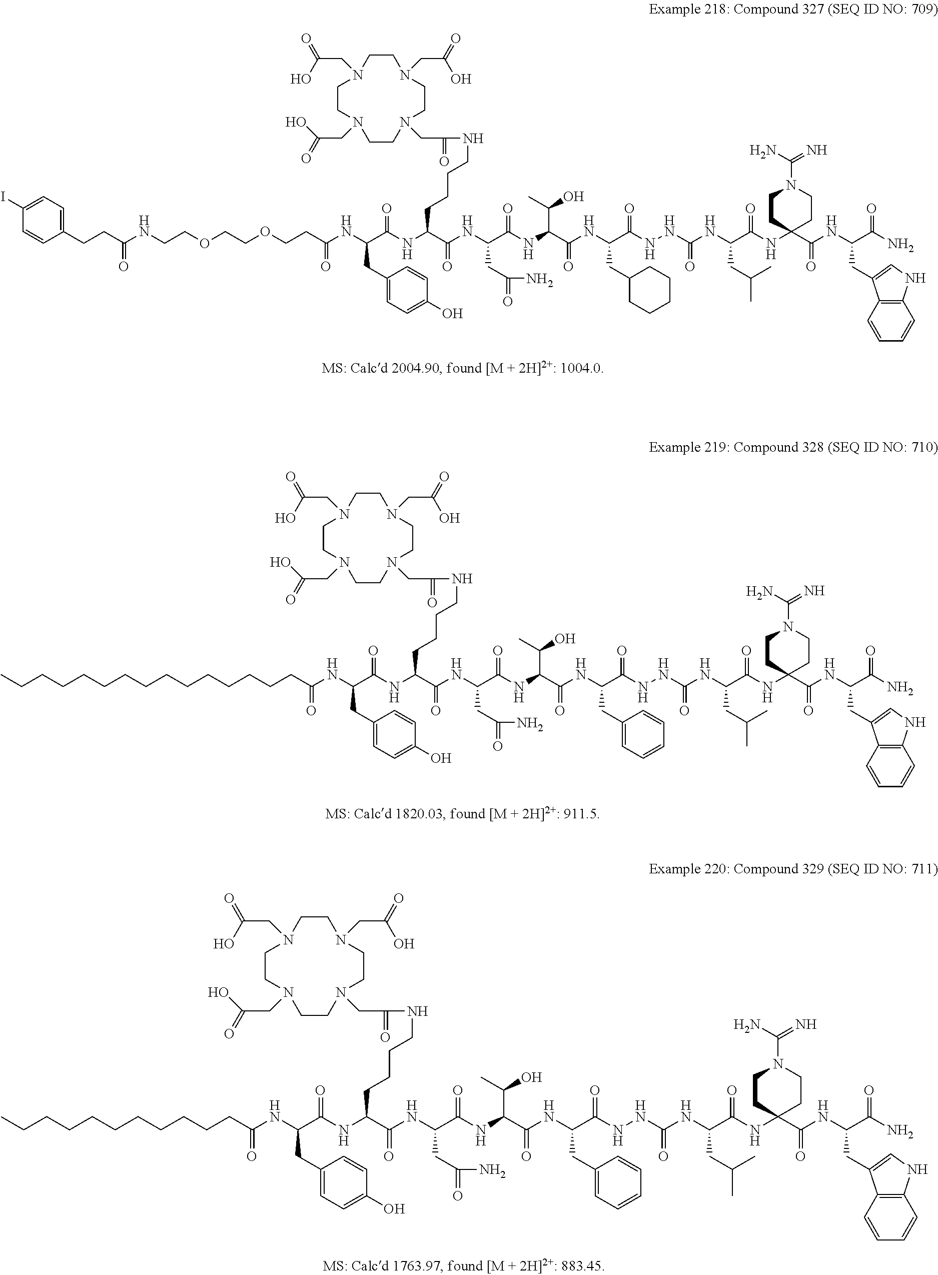

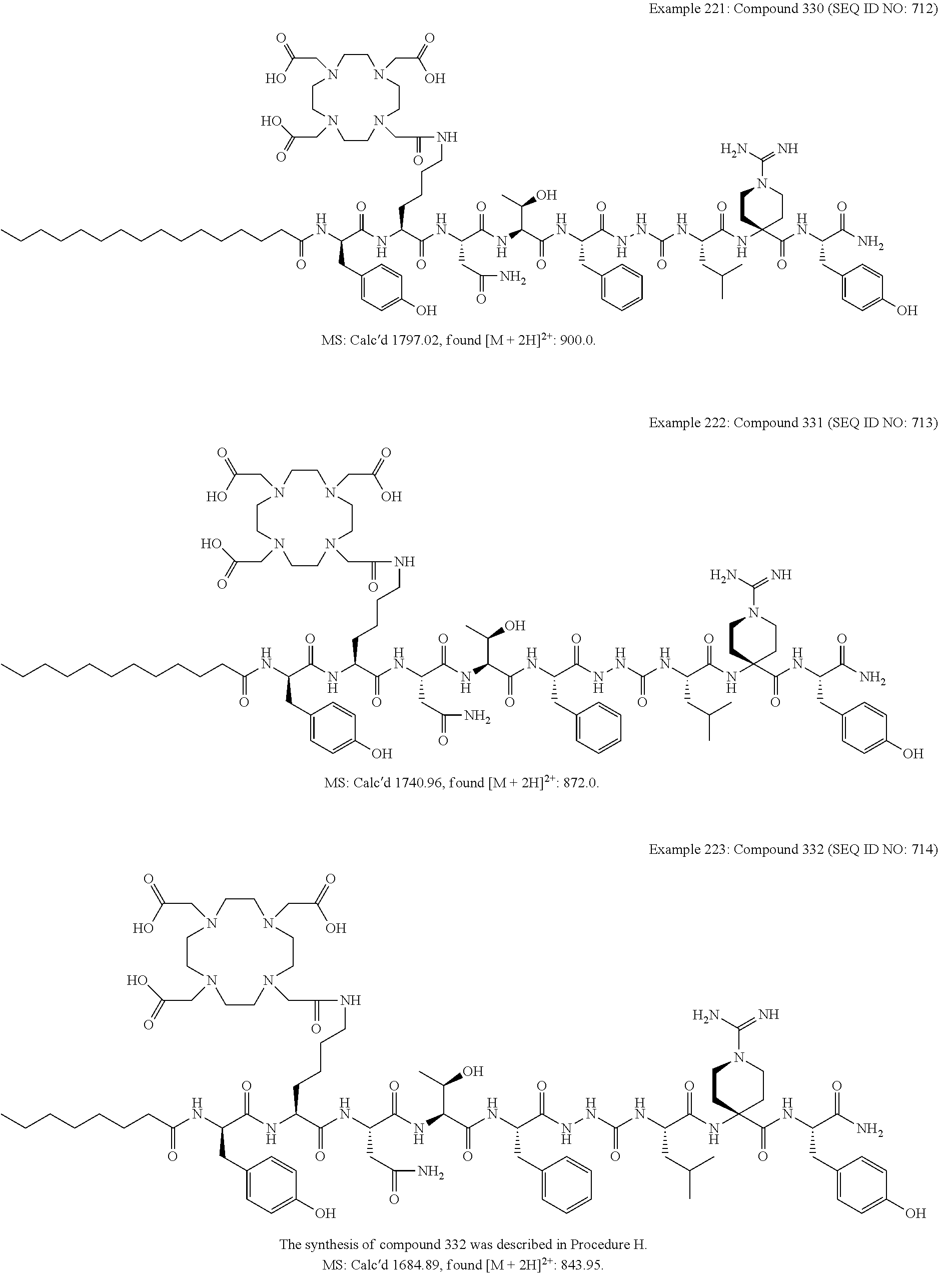

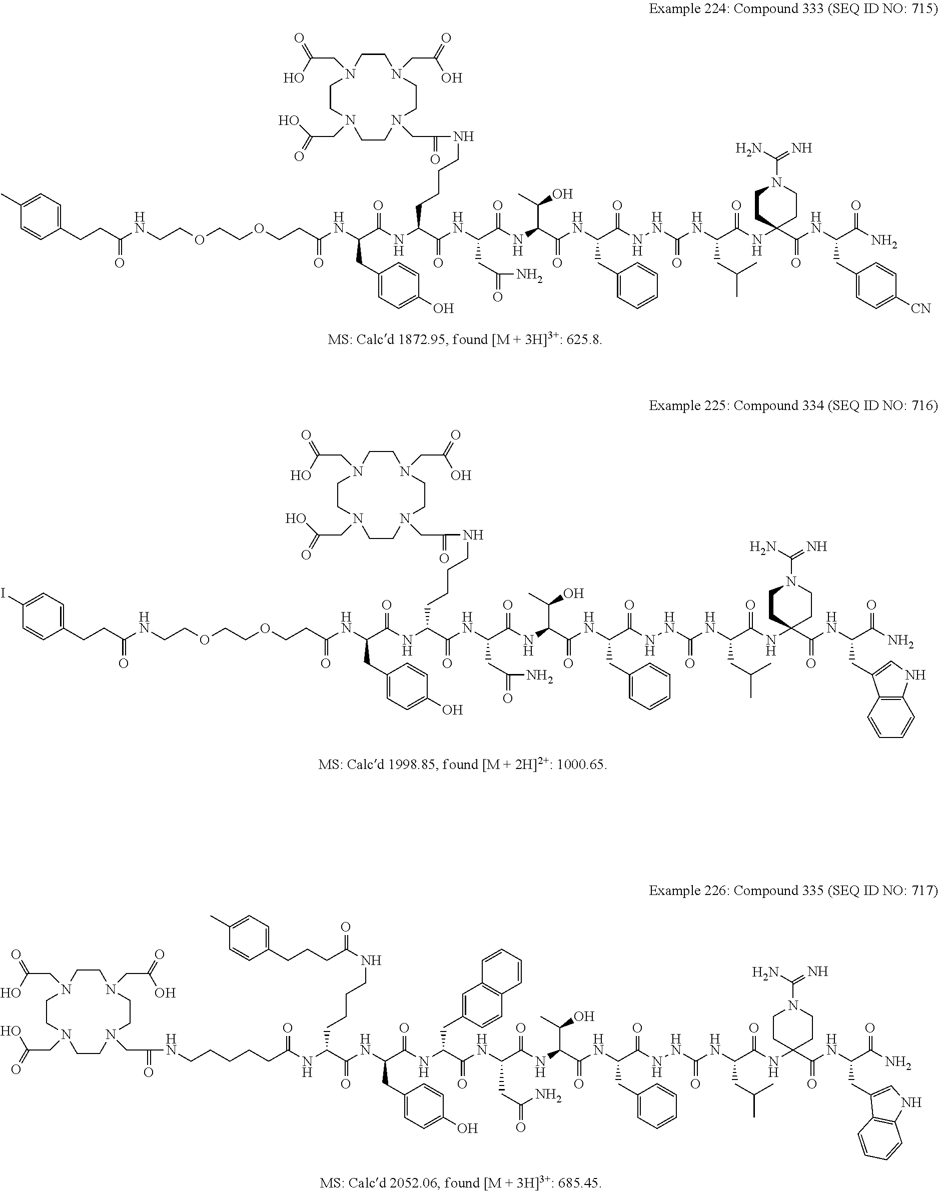

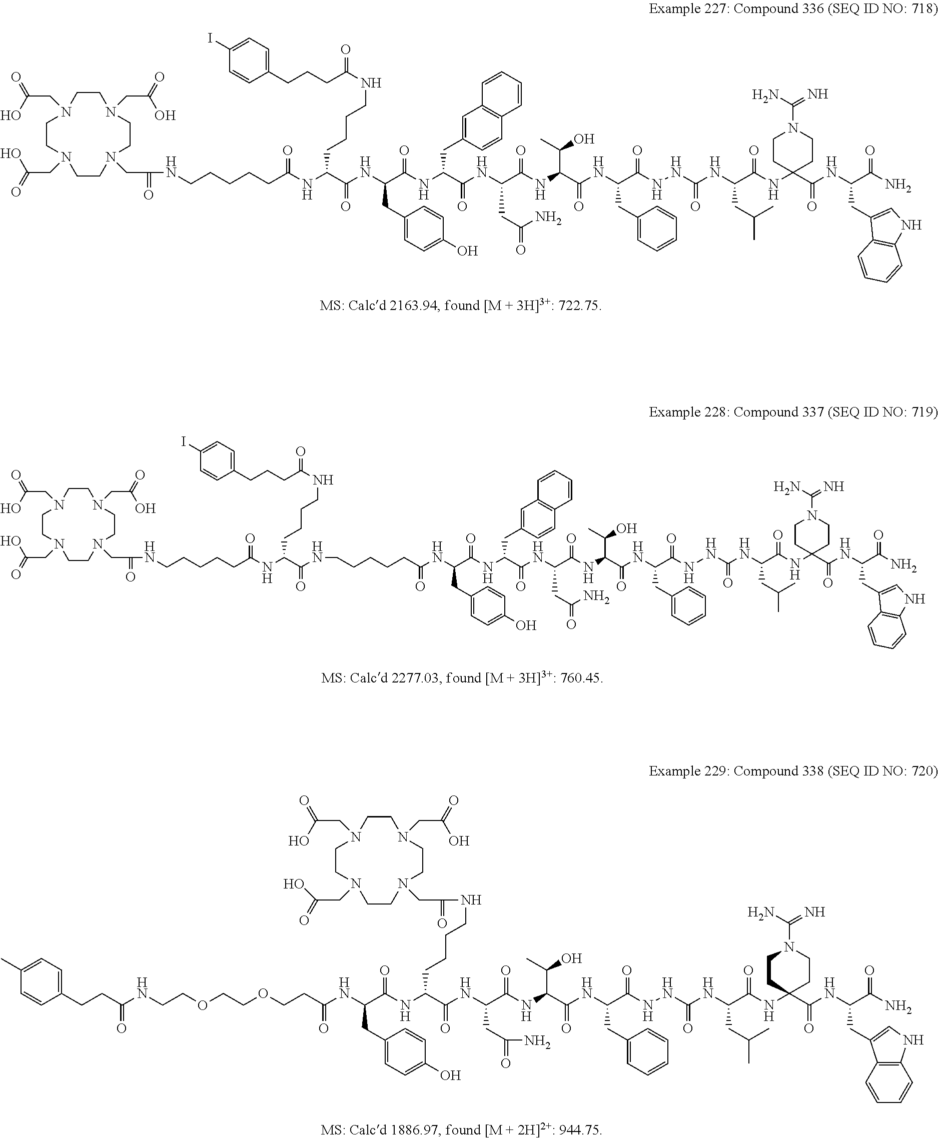

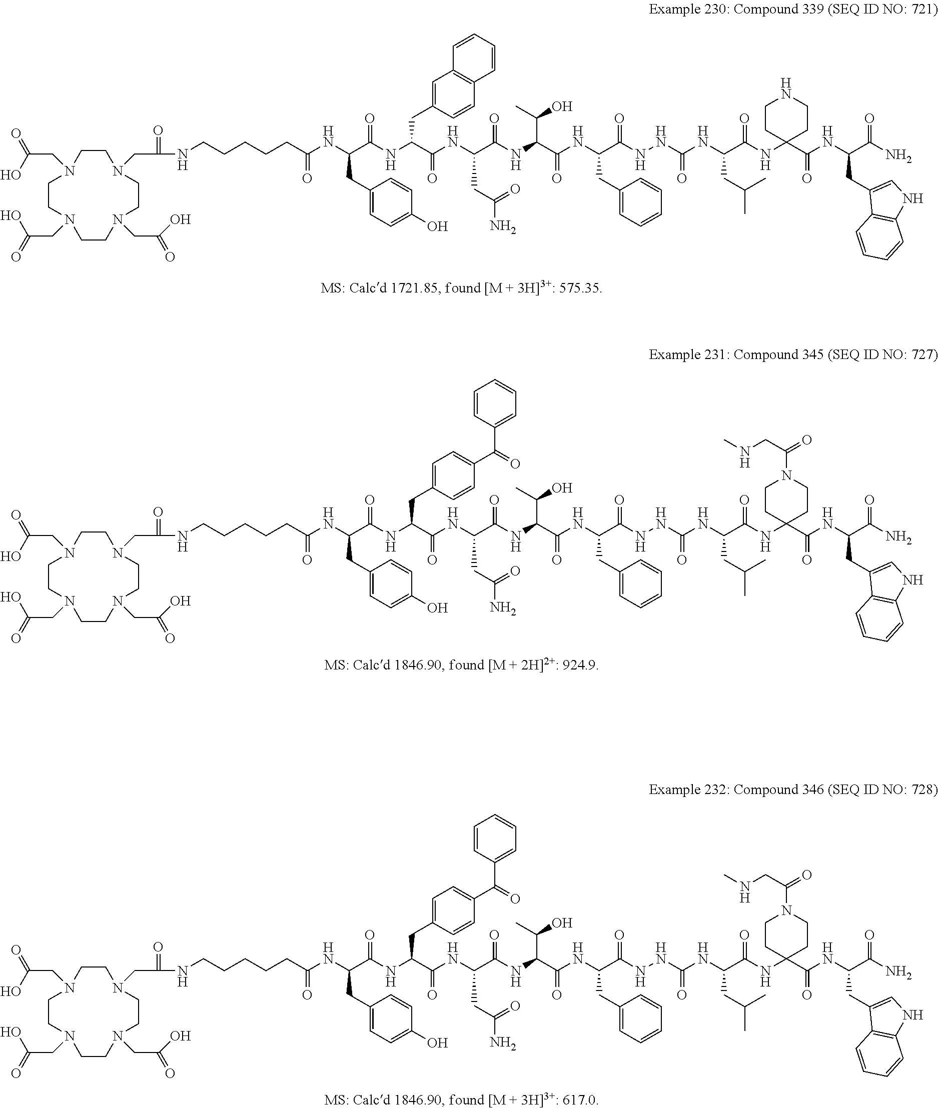

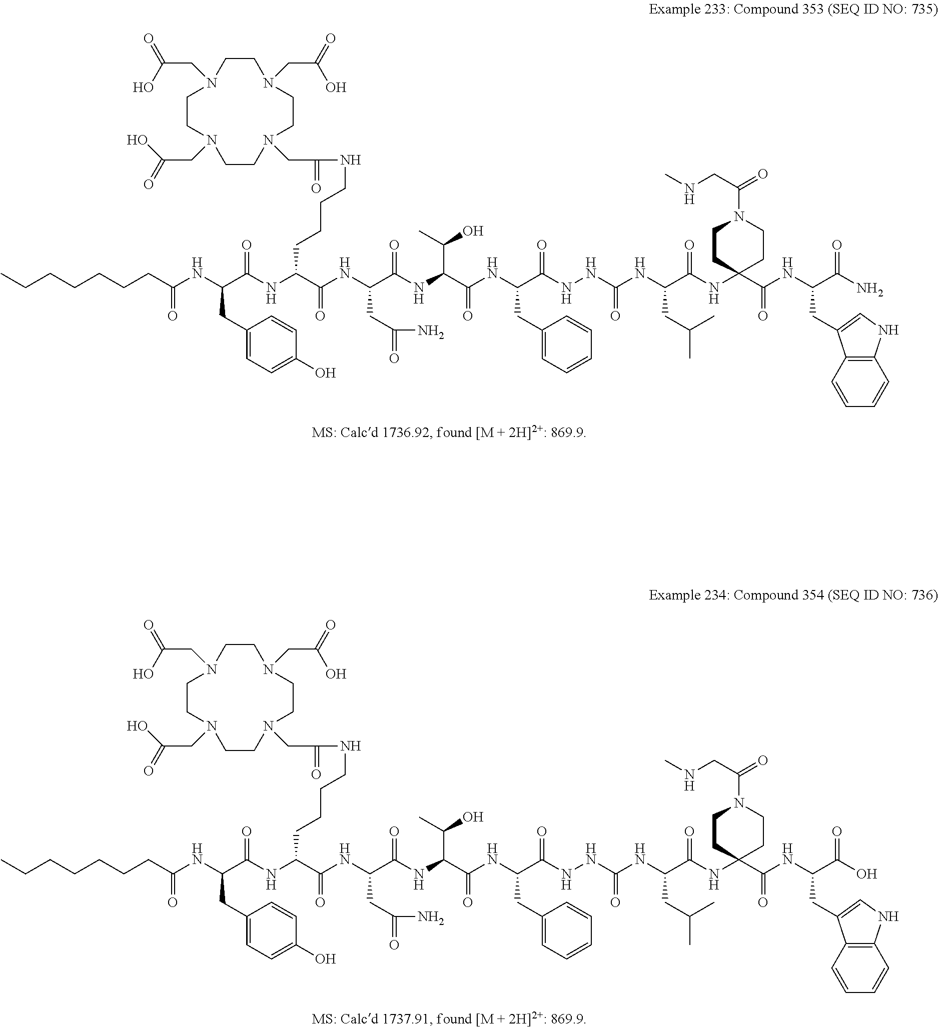

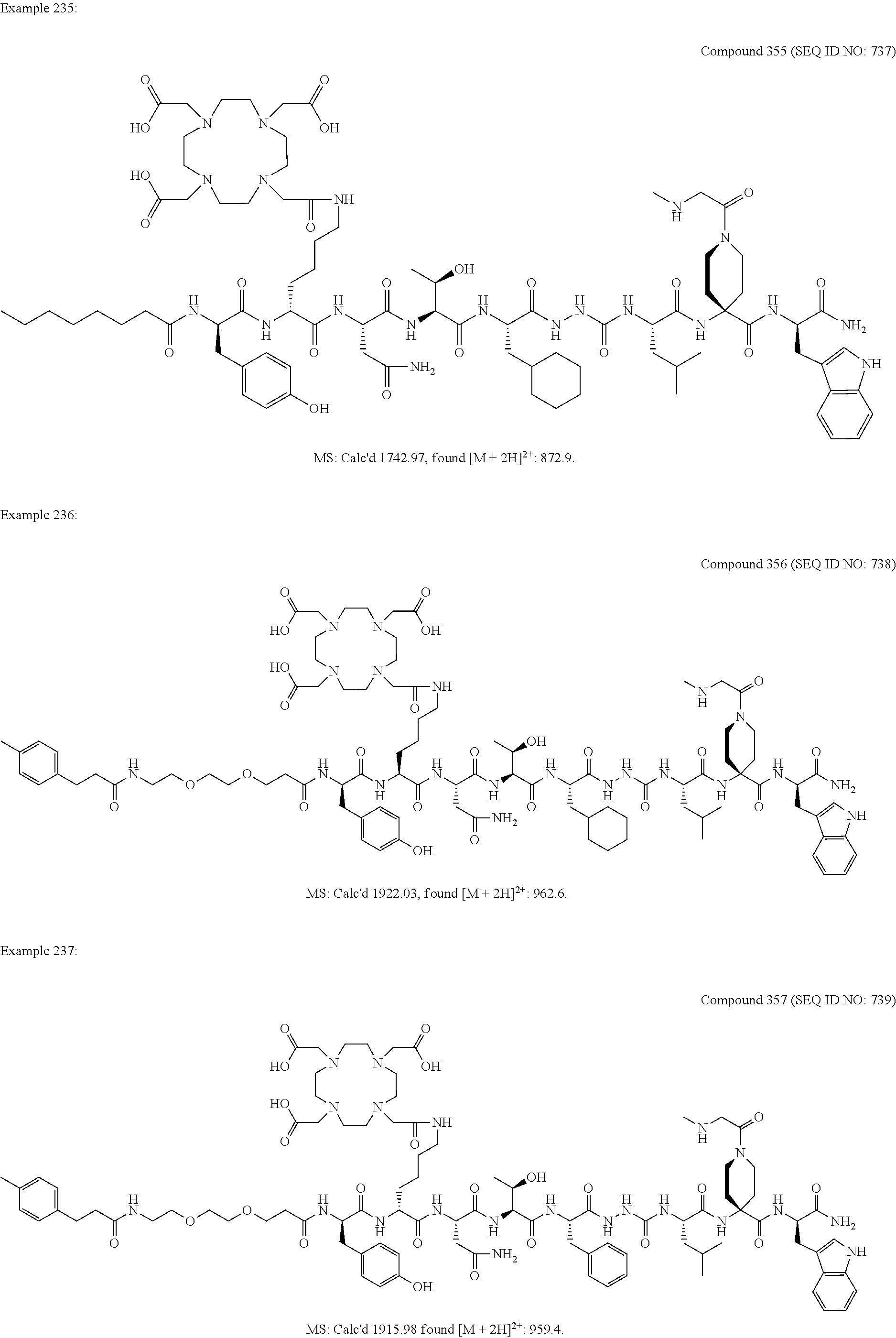

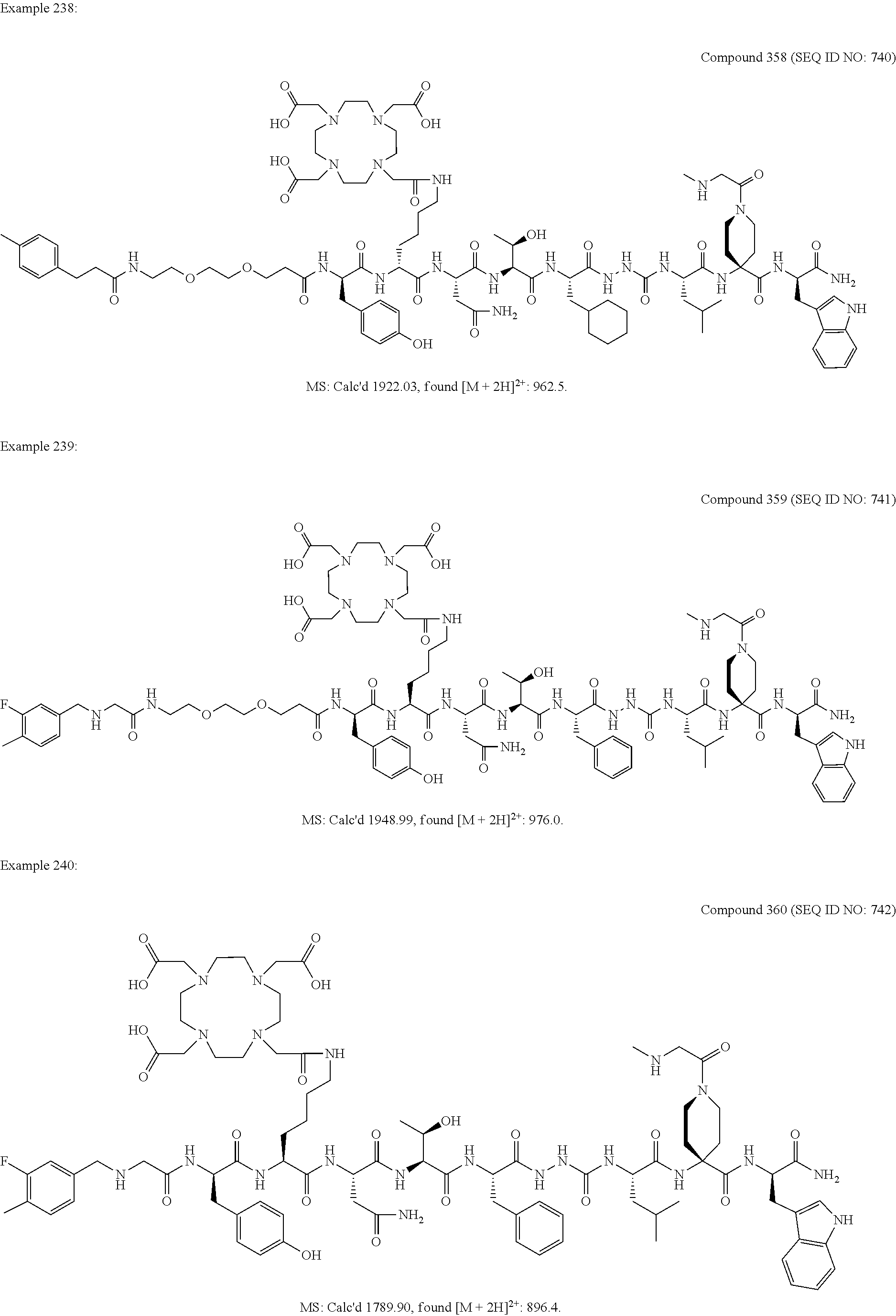

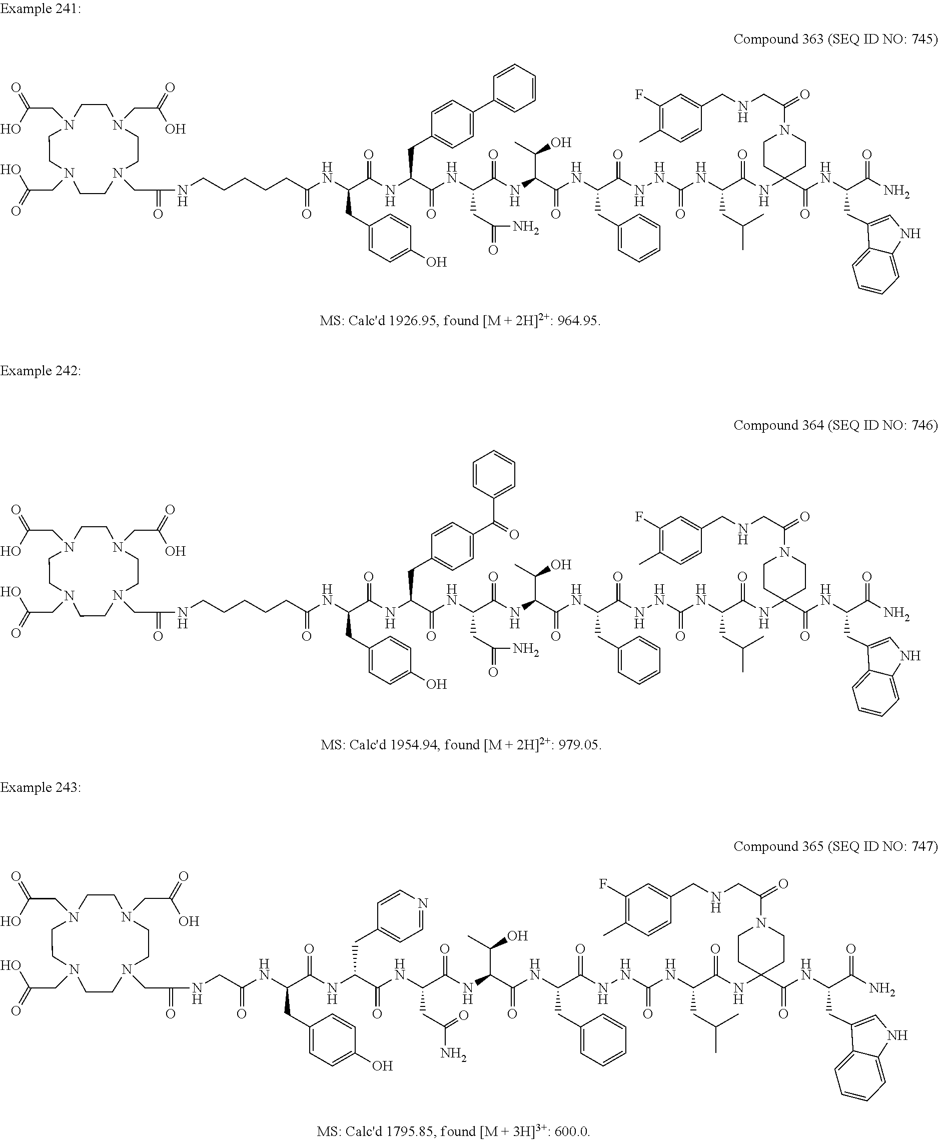

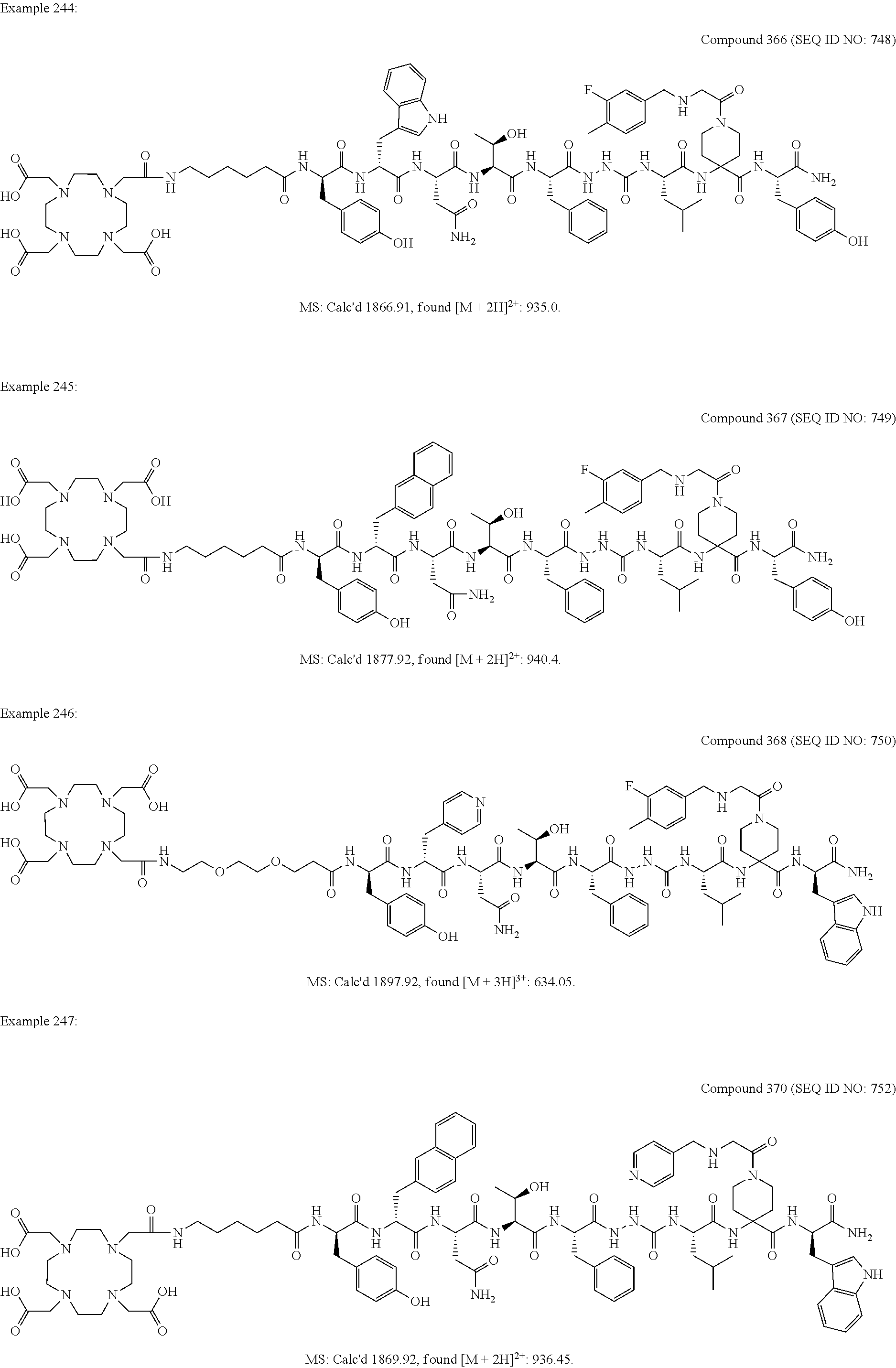

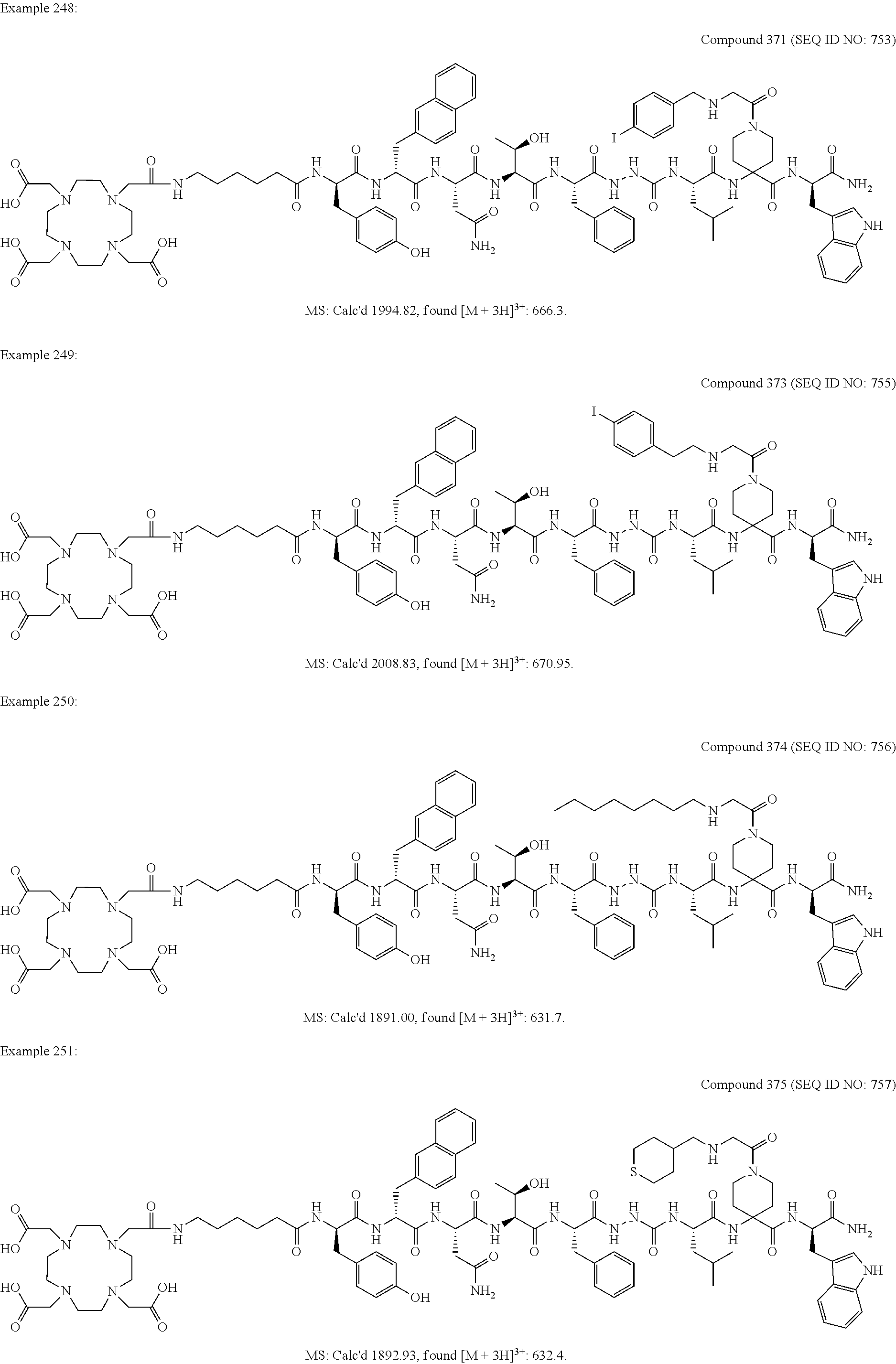

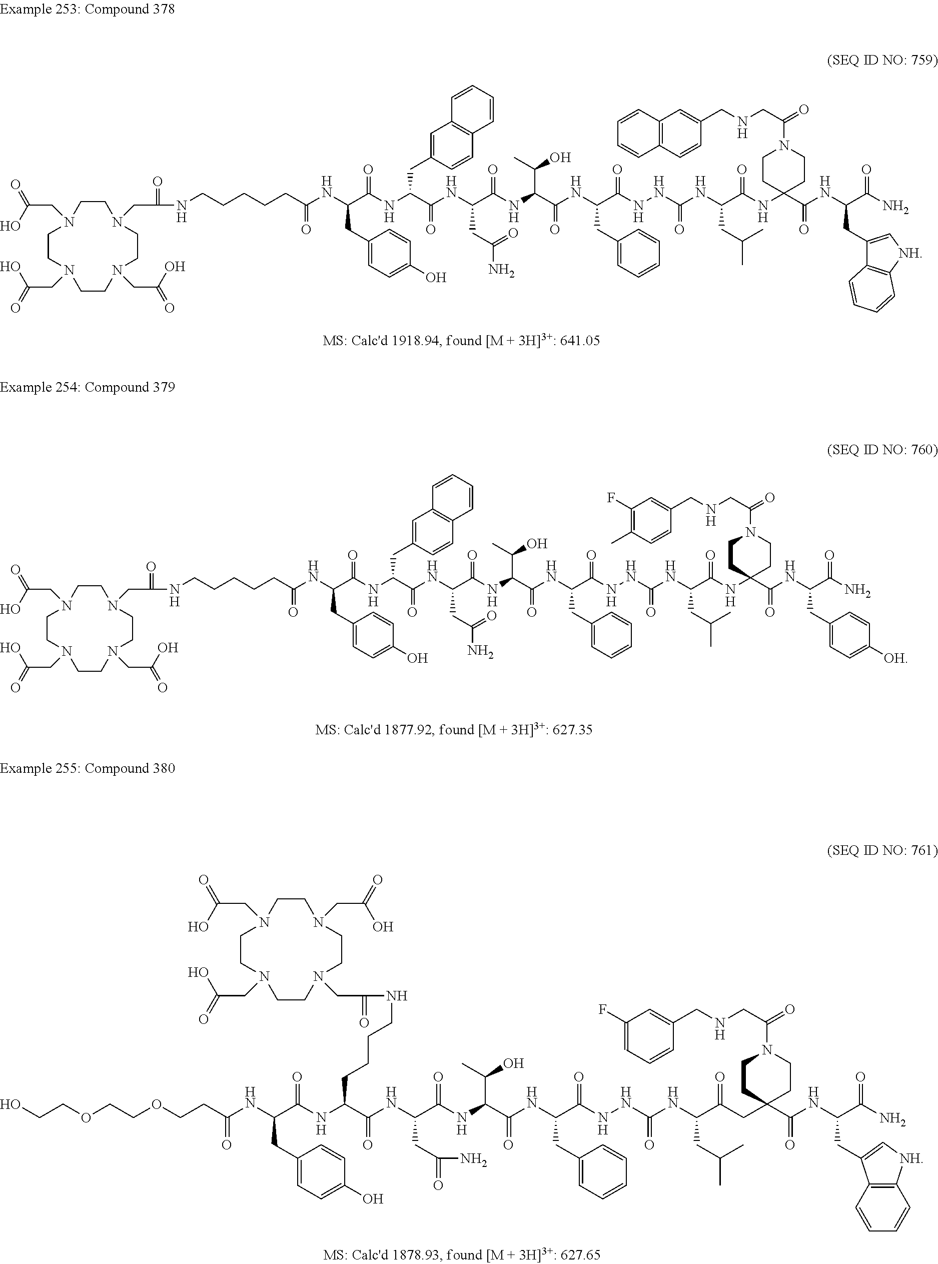

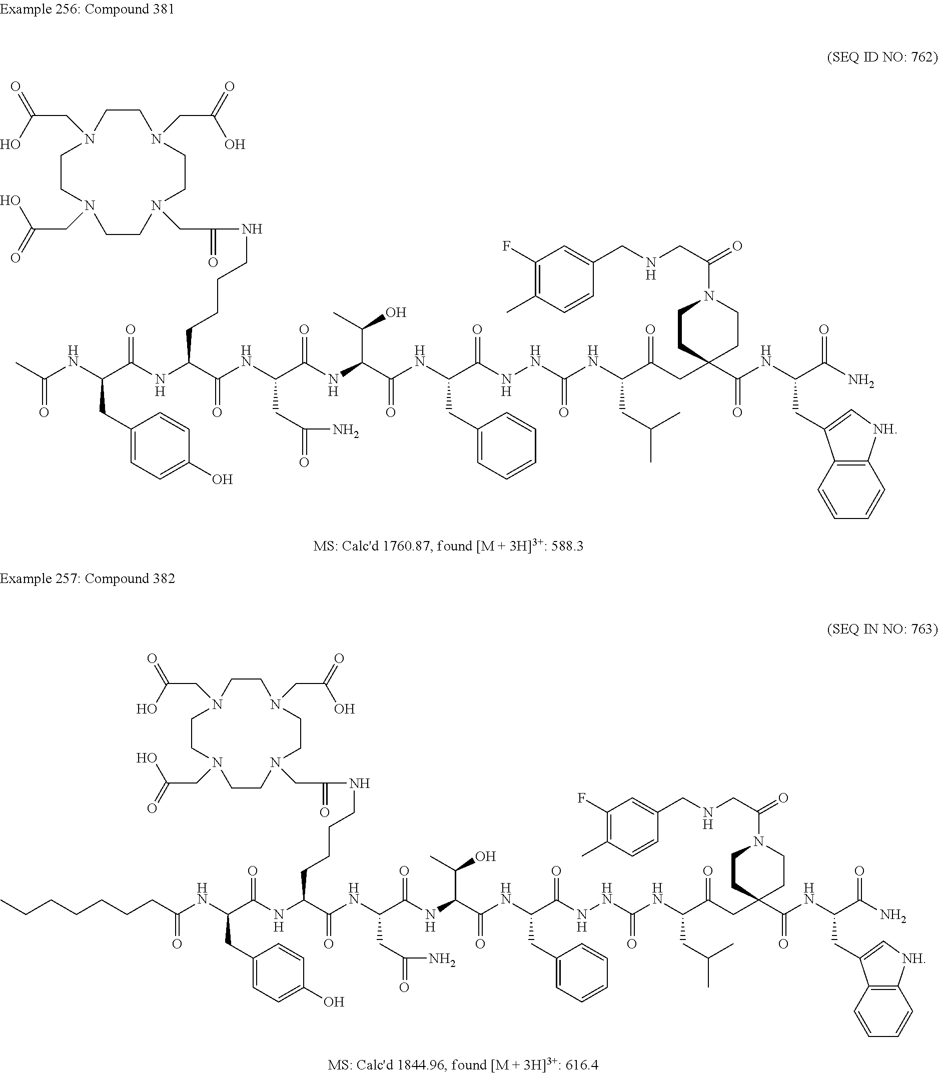

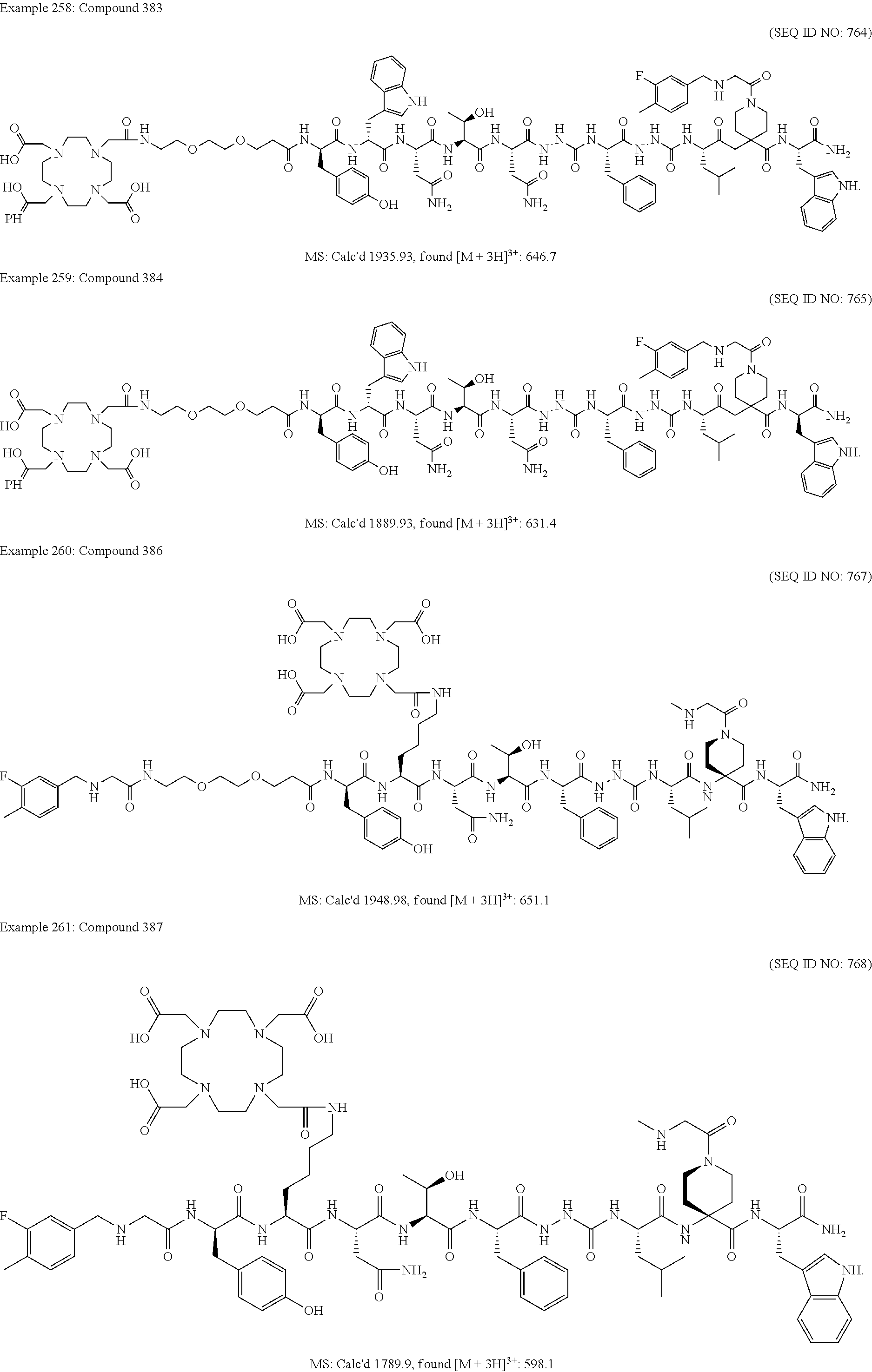

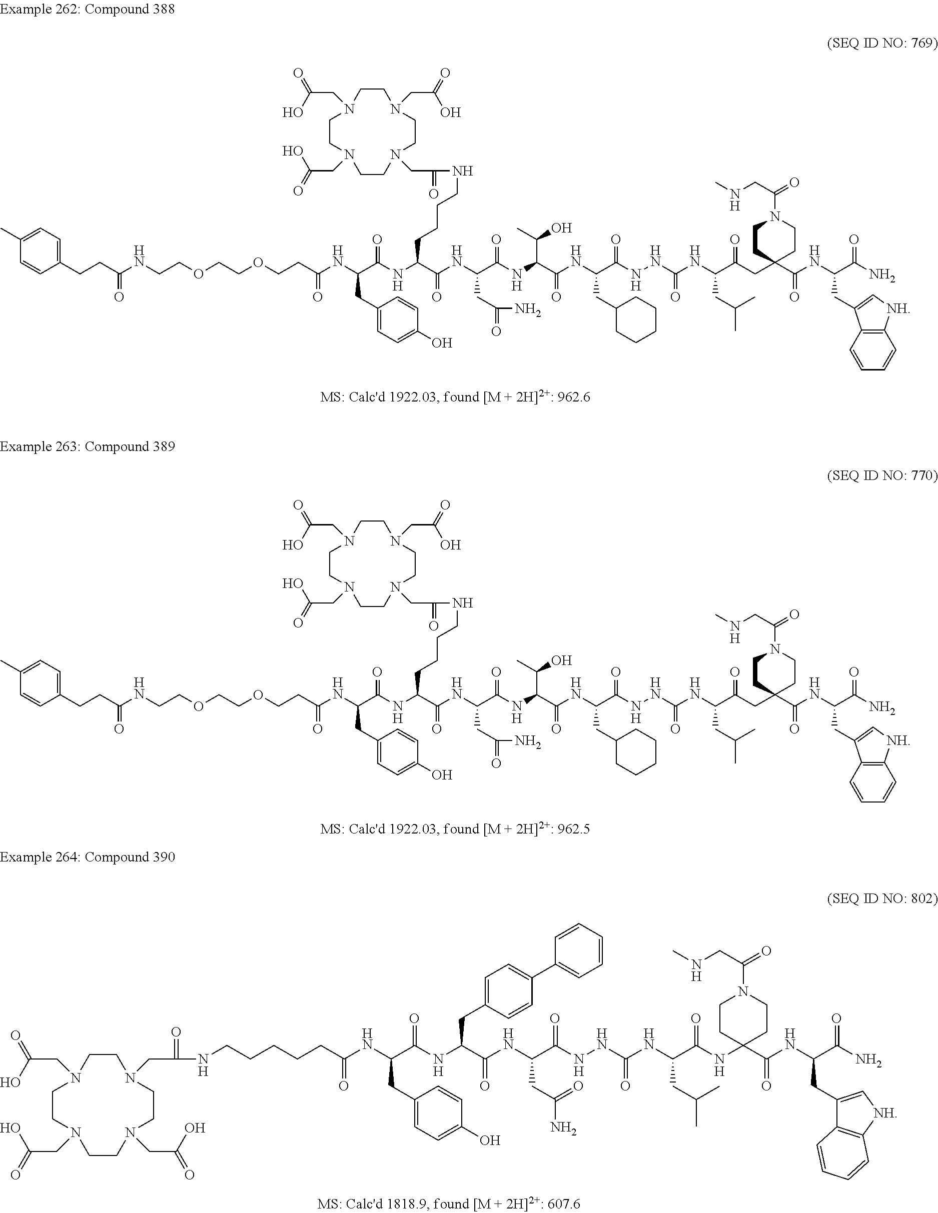

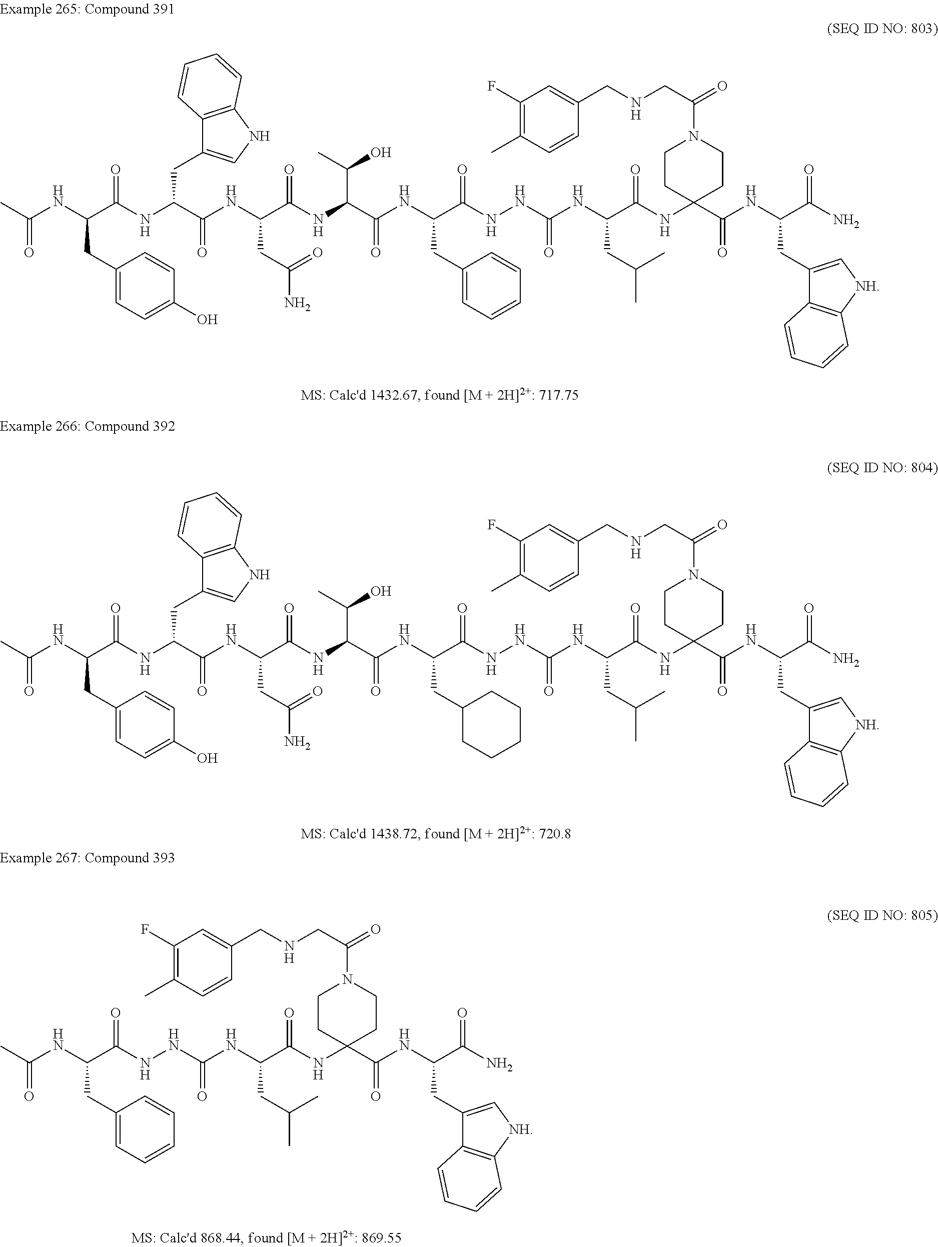

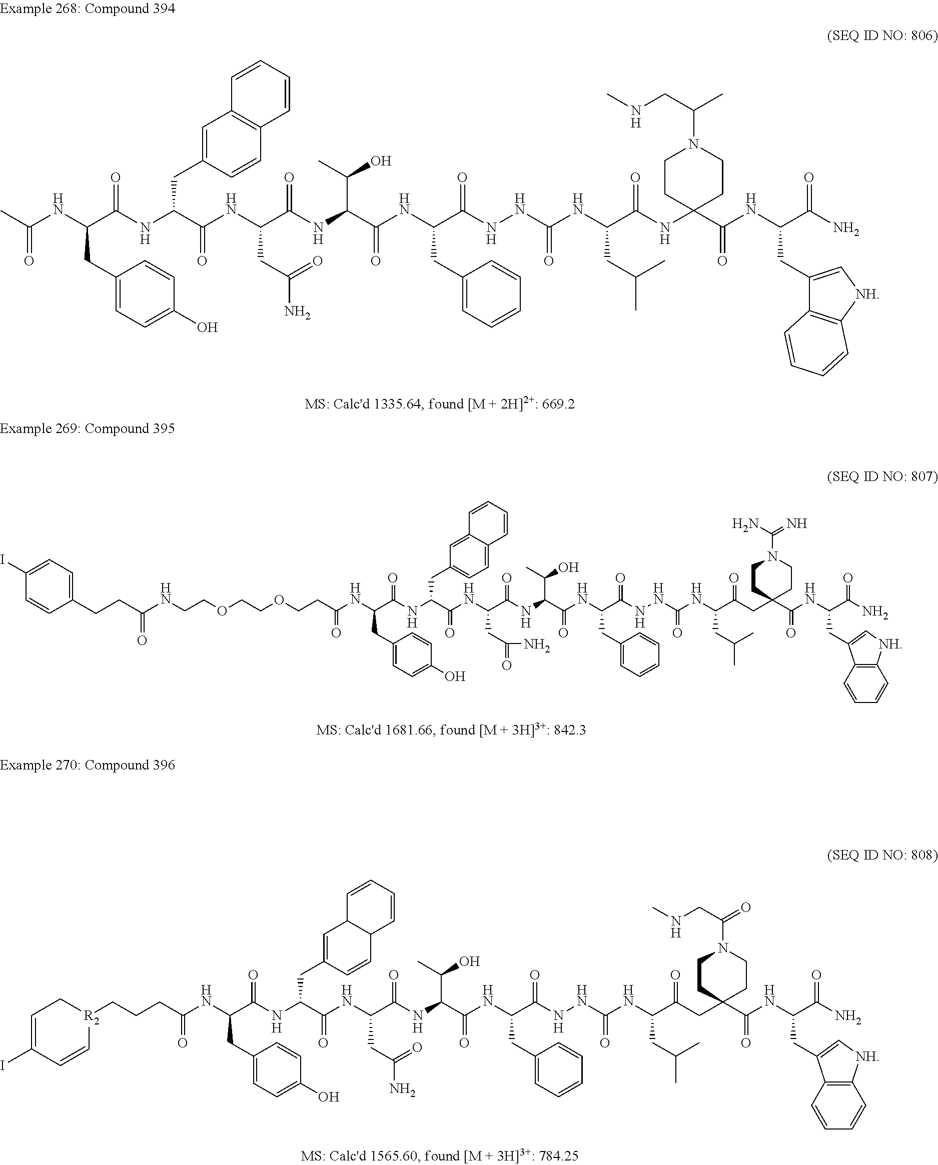

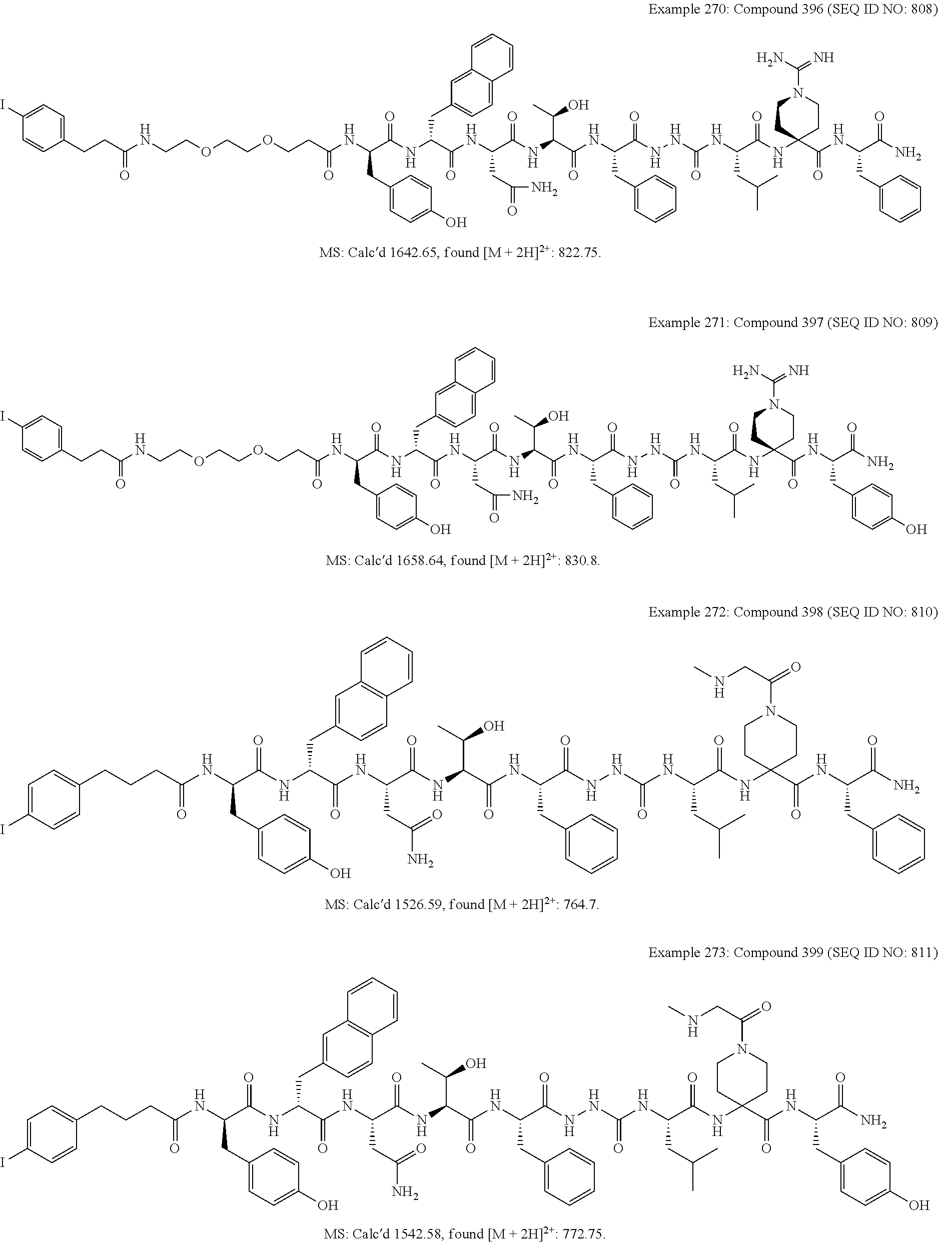

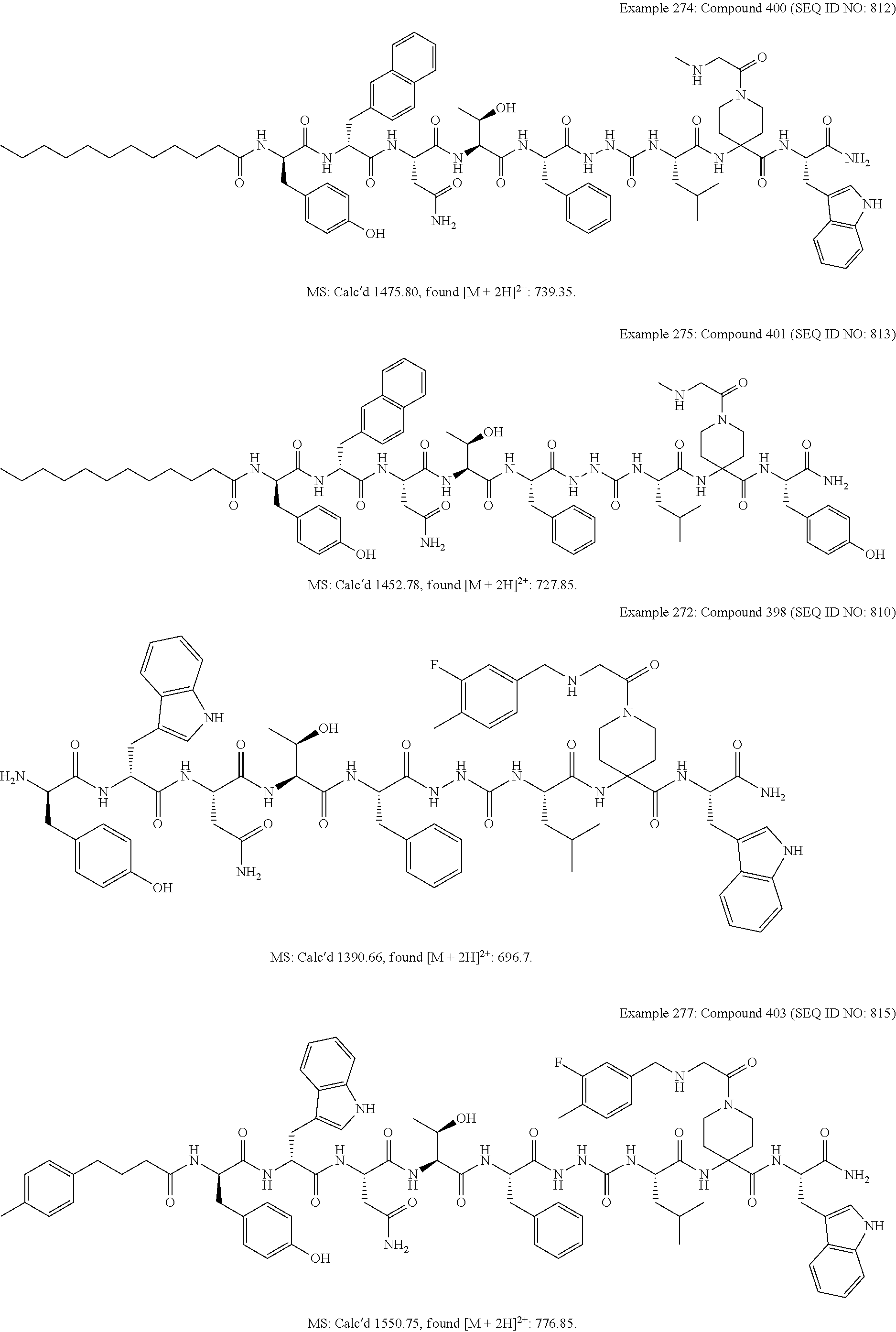

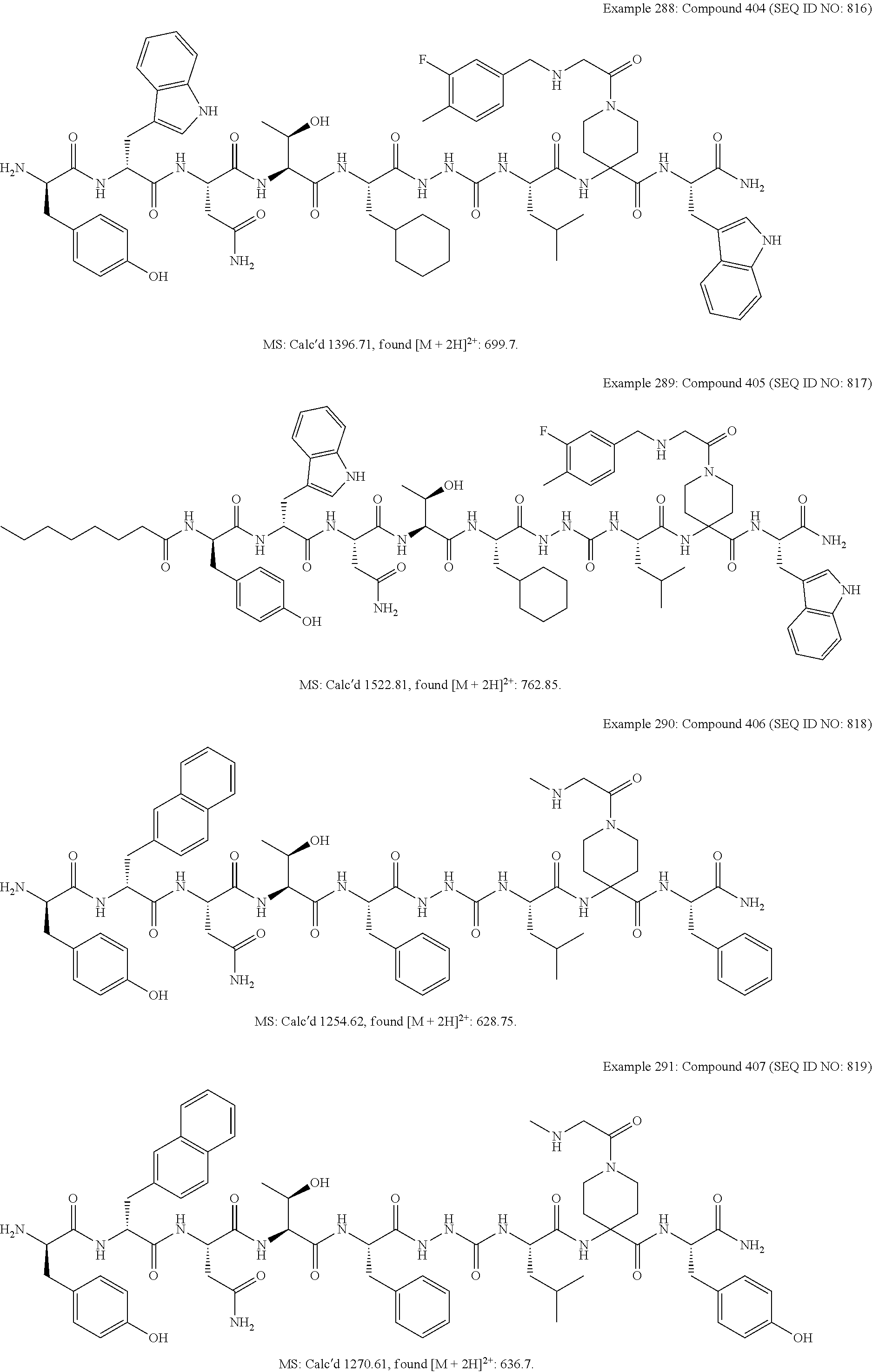

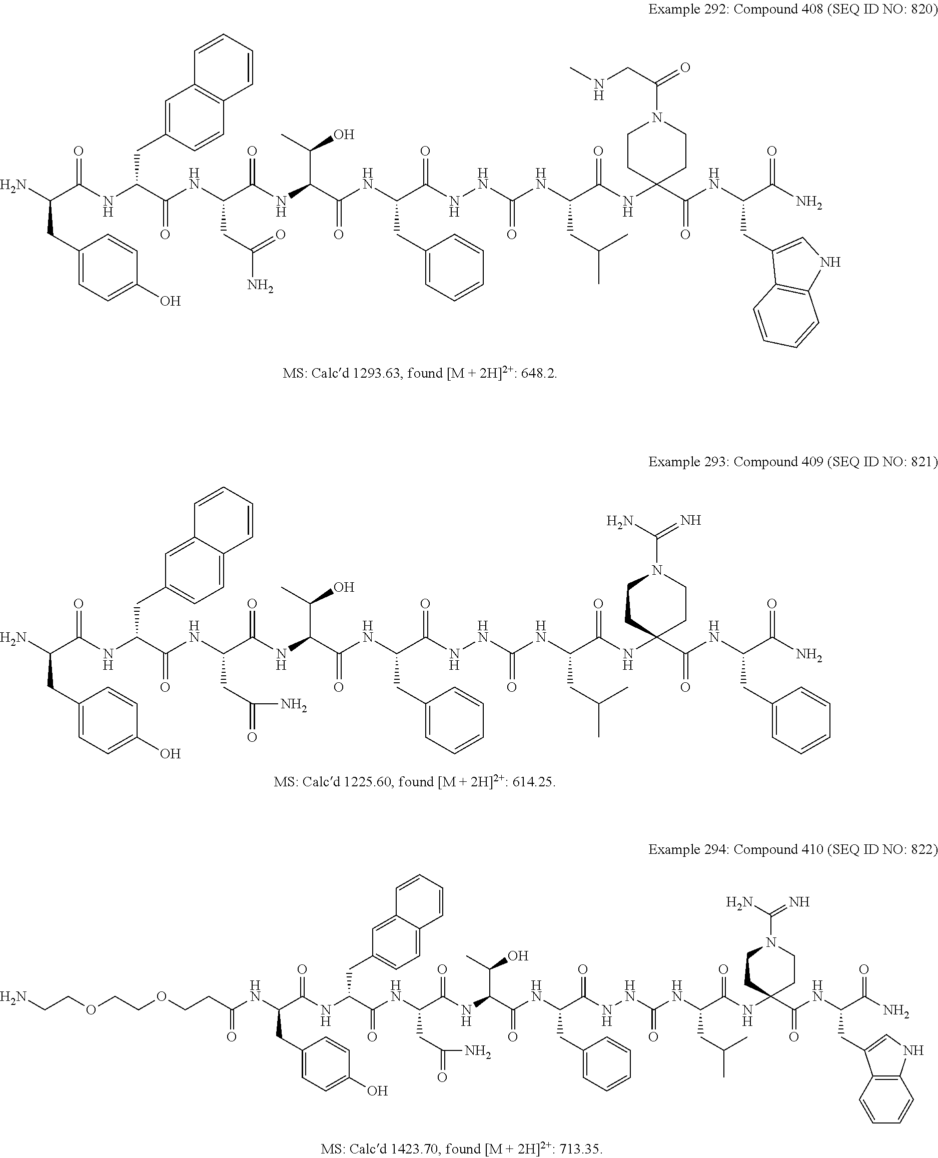

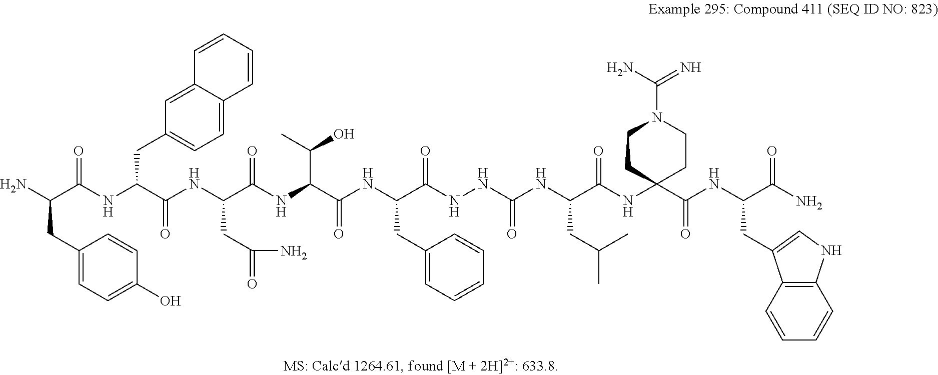

### Example A-1: Parenteral Pharmaceutical Composition

To prepare a parenteral pharmaceutical composition suitable for administration by injection (subcutaneous, intravenous), 0.001-500 mg of a compound Formula (I), or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Biology Examples

### Example B-1: Human KISS1R Binding Assay

### Membrane Preparation

Crude membrane fractions are prepared from Flp-In T-Rex 293 (Thermo Fisher) cell line expressing the human kisspeptin receptor. The cells are grown to 85-100% confluence on standard tissue culture dishes in Dulbecco's Modified Eagles Medium (DMEM) (Corning) supplemented with 10% FBS (Gemini), 1% penicillin-streptomycin-glutamine (Gibco), 500 μg/mL hygromycin, and 15 μg/mL blastacidin. 48 hrs prior to preparing membranes 10 μg/ml of tetracycline is added to the culture media to induce receptor expression. To prepare membranes, cells are scraped and collected in 1× Dulbecco's phosphate buffered saline (Corning) and then pelleted at 1000 RPMs. The cell pellet is reconstituted in membrane preparation buffer (20 mM HEPES, 6 mM MgCl₂ and 1 mM EGTA, protease inhibitor tablets (Pierce), pH 7.4) and placed in a cell disruption vessel (Parr Instrument company) at 1000 PSI for 30 min on ice. The pressurized contents are then released and spun down at 1000 RPMs and the supernatant is collected and further centrifuged at 15,000 RPMs to pellet the membranes. The membrane pellet is resuspended in membrane preparation buffer, snap frozen and stored at −80° C. for later use.

### Human Kisspeptin Binding Assay Protocol:

The human kisspeptin membrane binding assay utilizes the following components: radiolabel [¹²⁵I]-metastin (45-54) (human) (Perkinhmer), crude Flp-In T-Rex 293 kisspeptin membranes, and competing small molecule and peptide ligands. The assay is initiated by combining in assay buffer (50 mM TrisHCl, 5 mM MgCl₂, 2 mM EGTA, 10 mM CaCl₂, 0.1% BSA, pH 7.4) a dose response of competing ligand (final concentrations are typically 0-10,000 nM), 5-10 μg of Flp-In T-Rex 293 kisspeptin membranes, and 0.2 n M [¹²⁵I]-metastin (45-54) in a 96-well assay plate and allowed to incubate 90 minutes at room temperature. Assay contents are then filtered through unifilter CF/C: microplates (PerkinElmer) pre-soaked with 0.5% BSA and washed with 9×400 μL of cold wash buffer (10 mM HEPES, 500 mM NaCl, 0.1% Tween-20, pH7.4). Assay plates are read using a Microbeta² (PerkinElmer) and Ki values for compounds are determined using a GraphPad Prism 9.3 non-linear regression analysis.

Illustrative biological activity of compounds is demonstrated in Table G. The metal complexes in Table G comprise nonradioactive gallium, indium, and lutetium.

TABLE G
Representative Binding Activity

| | hKiSSR Binding Ki |
|---|---|
| Compound | (90 min, nM) |
| 1 | A |
| 1-In | A |
| 1-Lu | A |
| 1-Ga | A |
| 2 | A |
| 2-In | A |
| 3 | C |
| 5 | B |
| 6 | A |
| 6-In | A |
| 7 | B |
| 8 | A |
| 9 | A |
| 10 | A |
| 11 | A |
| 12 | B |
| 13 | B |
| 14 | B |
| 15 | C |
| 16 | C |
| 17 | A |
| 18 | B |
| 19 | B |
| 20 | A |
| 21 | B |
| 22 | A |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | C |
| 39 | C |
| 49 | C |
| 50 | A |
| 51 | A |
| 55 | A |
| 57 | A |
| 58 | A |
| 64 | A |
| 91 | A |
| 104 | A |
| 105 | A |
| 105-In | A |
| 106 | A |
| 107 | A |
| 108 | A |
| 109 | A |
| 110 | A |
| 113 | A |
| 114 | A |
| 115 | A |
| 116 | A |
| 117 | A |
| 118 | A |
| 118-Lu | A |
| 119 | A |
| 120 | A |
| 121 | B |
| 123 | C |
| 130 | A |
| 131 | A |
| 132 | A |
| 133 | A |
| 134 | B |
| 135 | C |
| 136 | C |
| 137 | A |
| 138 | B |
| 139 | B |
| 140 | A |
| 141 | B |
| 142 | A |
| 146 | A |
| 147 | C |
| 148 | B |
| 149 | A |
| 151 | A |
| 156 | A |
| 158 | A |
| 159 | A |
| 160 | A |
| 161 | A |
| 164 | A |
| 165 | A |
| 166 | A |
| 167 | A |
| 168 | A |
| 169 | A |
| 170 | A |
| 171 | B |
| 176 | A |
| 181 | A |
| 182 | A |
| 183 | A |
| 184 | A |
| 185 | A |
| 186 | A |
| 187 | A |
| 188 | B |
| 189 | C |
| 192 | A |
| 193 | A |
| 193-In | A |
| 195 | A |
| 196 | A |
| 196-In | A |
| 197 | A |
| 198 | A |
| 199 | C |
| 201 | A |
| 201-In | A |
| 202 | B |
| 203 | A |
| 204 | C |
| 205 | A |
| 206 | A |
| 213 | A |
| 214 | A |
| 215 | C |
| 216 | A |
| 217 | A |
| 218 | A |
| 221 | A |
| 222 | A |
| 226 | B |
| 230 | B |
| 231 | B |
| 232 | A |
| 233 | A |
| 234 | A |
| 235 | A |
| 236 | A |
| 237 | A |
| 238 | A |
| 239 | C |
| 240 | A |
| 241 | A |
| 242 | A |
| 244 | A |
| 245 | A |
| 246 | B |
| 247 | A |
| 248 | C |
| 249 | A |
| 250 | A |
| 251 | C |
| 252 | C |
| 253 | A |
| 254 | A |
| 255 | A |
| 256 | A |
| 258 | C |
| 259 | B |
| 260 | B |
| 261 | A |
| 264 | A |
| 266 | A |
| 269 | A |
| 270 | A |
| 271 | B |
| 272 | A |
| 273 | B |
| 275 | A |
| 276 | A |
| 277 | A |
| 278 | A |
| 279 | A |
| 280 | C |
| 281 | A |
| 282 | C |
| 283 | B |
| 284 | B |
| 285 | A |
| 286 | A |
| 287 | B |
| 288 | A |
| 289 | A |
| 290 | A |
| 291 | A |
| 292 | A |
| 293 | A |
| 294 | A |
| 295 | A |
| 296 | A |
| 297 | A |
| 298 | A |
| 299 | A |
| 300 | A |
| 301 | A |
| 302 | A |
| 304 | A |
| 305 | A |
| 306 | A |
| 307 | A |
| 308 | A |
| 309 | A |
| 310 | A |
| 311 | A |
| 312 | B |
| 313 | A |
| 314 | A |
| 315 | A |
| 316 | A |
| 317 | A |
| 318 | A |
| 319 | C |
| 320 | A |
| 321 | A |
| 322 | A |
| 323 | A |
| 324 | A |
| 325 | A |
| 326 | A |
| 327 | A |
| 328 | A |
| 329 | A |
| 330 | A |
| 331 | A |
| 332 | A |
| 333 | A |
| 334 | A |
| 335 | A |
| 336 | A |
| 337 | A |
| 338 | A |
| 339 | A |
| 345 | A |
| 346 | A |
| 353 | A |
| 354 | A |
| 355 | A |
| 356 | A |
| 357 | A |
| 358 | A |
| 359 | A |
| 360 | A |
| 363 | A |
| 364 | A |
| 365 | A |
| 366 | A |
| 367 | A |
| 368 | A |
| 370 | C |
| 371 | A |
| 373 | B |
| 374 | A |
| 375 | C |
| 376 | B |
| 378 | A |
| 379 | A |
| 380 | A |
| 381 | A |
| 382 | A |
| 383 | A |
| 384 | A |
| 386 | A |
| 387 | A |
| 388 | A |
| 389 | A |
| 390 | A |
| 391 | A |
| 392 | A |
| 393 | A |
| 394 | A |
| 395 | A |
| 396 | A |
| 397 | A |
| 398 | A |
| 399 | A |
| 400 | A |
| 401 | A |
| 402 | A |
| 403 | A |
| 404 | A |
| 405 | A |
| 406 | A |
| 407 | A |
| 408 | A |
| 409 | A |
| 410 | A |
| 411 | A |

*A is <10 nM; B is 10-100 nM; C is >100 nM

### Example B-2: Biodistribution of ¹¹¹In[In] Complex of Compound 1 in Mouse Tumor Model

TABLE H
Study outline

| | | Dose | Dose | |
|---|---|---|---|---|
| | N | (MBq/ | (nmol/ | Time |
| Treatment | value | Mouse) | mouse) | (h) |
| 111In[In]-Compound 1 | 4 | 5-7 | 1 | 0.5 |
| 111In[In]-Compound 1 | 4 | 5-7 | 1 | 2 |
| 111In[In]-Compound 1 | 4 | 5-7 | 1 | 5 |
| 111In[In]-Compound 1 | 4 | 5-7 | 1 | 24 |
| 111In[In]-Compound 1 | 4 | 5-7 | 1 | 72 |
| 111In[In]-Compound 1 + Cold- | 4 | 5-7 | 1 + | 2 h |
| Compound 1 | | 0 | 100 nm | |

*Treatment schedule is Q1Dx1(IV)

Study Details: 24 h prior to the start of the biodistribution Compound 1 (CMPD 1) was radiolabeled.

Prior to initiation of the biodistribution study, female Swiss nude mice were subcutaneously inoculated with 10×10⁶ human cancer cells.

When tumors were between 150-300 mm³, animals were randomized and received a single IV injection of 200 μL into the caudal vein via a catheter with 5-7 MBq of ¹¹¹In[In] Compound 1 (1 mmol) per animal. For the competition study arm, 1 mmol of ¹¹¹In[In]-Compound 1 was co-injected with 100 mmol of unlabeled Compound 1.

After drug administration, animals were euthanized at timepoints (0.5 h, 2 h, 5 h, 24 h and 72 h) and organs (blood, heart, muscle, lungs, intestines, spleen, bone, brain, tail, carcass, kidneys, liver and tumor) were collected, weighed, and y radioactivity assessed in each organ/tissue. Activity was quantitated and expressed as % ID/g (Percentage of Initial Dose/gram of tissue).

### Example B3-3: Biodistribution of ¹¹¹In[In] Complex of Compound 6 in Mouse Tumor Model

TABLE I
Study outline

| | | Dose | Dose | |
|---|---|---|---|---|
| | N | (MBq/ | (nmol/ | Time |
| Treatment | value | Mouse) | mouse) | (h) |
| ¹¹¹In[In]-Compound 6 | 3 | 5-7 | 1 | 0.75 |
| ¹¹¹In[In]-Compound 6 | 3 | 5-7 | 1 | 2 |
| ¹¹¹In[In]-Compound 6 | 3 | 5-7 | 1 | 5 |
| ¹¹¹In[In]-Compound 6 | 3 | 5-7 | 1 | 24 |
| ¹¹¹In[In]-Compound 6 | 3 | 5-7 | 1 | 44 |
| ¹¹¹In[In]-Compound 6 + Cold- | 3 | 5-7 | 1 + | 2 h |
| Compound 6 | | 0 | 100 nm | |

*Treatment schedule is Q1Dx1(IV)

Study Details: 24 h prior to the start of the biodistribution Compound 6 was radiolabeled as described

Prior to initiation of the biodistribution study, female Swiss nude mice were subcutaneously inoculated with 10×106 human cancer cells.

When tumors were between 150-300 mm³, animals were randomized and received a single IV injection of 200 μL into the caudal vein via a catheter with 5-7 MBq of ¹¹¹In[In] Compound 6 (1 mmol) per animal. For the competition study arm, 1 mmol of ¹¹¹In[In]-Compound 6 was co injected with 100 mmol of unlabeled Compound-6.

After drug administration, animals were euthanized at timepoints (0.75 h, 2 h, 5 h, 24 h and 44 h) and organs (blood, carcass, gut, kidneys, liver, lungs, muscle, spleen, tail, and tumor) were collected, weighed, and 9, radioactivity assessed in each organ/tissue. Activity was quantitated and expressed as % ID/g (Percentage of Initial Dose/grain of tissue).

### Example 1-4: Biodistribution of ¹¹¹In[In] a Complex of Compound 105 in Mouse Tumor Model

TABLE J
Study outline

| | | Dose | Dose | |
|---|---|---|---|---|
| | N | (MBq/ | (nmol/ | Time |
| Treatment | value | Mouse) | mouse) | (h) |
| ¹¹¹In[In]-Compound 105 | 4 | 5-7 | 1 | 0.5 |
| ¹¹¹In[In]-Compound 105 | 4 | 5-7 | 1 | 2 |
| ¹¹¹In[In]-Compound 105 | 4 | 5-7 | 1 | 5 |
| ¹¹¹In[In]-Compound 105 | 4 | 5-7 | 1 | 24 |
| ¹¹¹In[In]-Compound 105 | 4 | 5-7 | 1 | 44 |
| ¹¹¹In[In]-Compound 105 + Cold- | 4 | 5-7 | 1 + | 2 h |
| Compound 105 | | 0 | 100 nm | |

*Treatment schedule is Q1Dx1(IV)

Study Details: 24 h prior to the start of the biodistribution Compound 105 was radiolabeled

Prior to initiation of the biodistribution study, female Swiss nude mice were subcutaneously inoculated with 10×10⁶ human cancer cells.

When tumors were between 150-300 mm³, animals were randomized and received a single IV injection of 200 μL into the caudal vein via a catheter with 5-7 MBq of ¹¹¹In[In] Compound 105 (1 mmol) per animal. For the competition study arm, 1 mmol of ¹¹¹In[In]-Compound 105 was co injected with 100 mmol of unlabeled Compound 105.

After drug administration, animals were euthanized at timepoints (0.5 h, 2 b, 5 h, 24 h and 44 h) and organs (Blood, Carcass, Gut, Heart, Kidneys, Liver, Lungs, Muscle Spleen Tail, and Tumor) were collected, weighed, and ÿ radioactivity assessed in each organ/tissue. Activity was quantitated and expressed as % ID/g (Percentage of Initial Dose/gram of tissue).

### Example B-5: Safety and Dosimetry Study in Patients with Breast Cancer and Healthy Volunteers

A non-limiting example of a phase 1 safety and dosimetry study of ⁶⁸Ga-complex of a compound of Formula (I) in patients with breast, kidney cancer and non-small cell lung cancer is described below.

This is an open-label, first-in-human, Phase 1 study of ⁶⁸Ga-complex of a compound of Formula (I) designed to characterize its safety and biodistribution in patients diagnosed with breast cancer, clear cell renal cell carcinoma or non-small cell lung cancer. In some embodiments, a ⁶⁸Ga-complex of a compound of Formula (I) is used to localize KISS1R-expressing lesions and identify breast cancer, clear cell renal cell carcinoma or non-small cell lung cancer patients with KISS1R-expressing tumors who may benefit from treatment with KISS1R-targeting therapeutic agents, such as ¹⁷⁷Lu-complex of a compound of Formula (I). In healthy humans, KISS1R is found primarily in the hypothalamus, pituitary and placenta, and plays a role in regulating puberty and fertility. KISS1R is highly expressed in breast cancer, clear cell renal cell carcinoma and non-small cell lung cancer.

This study will enroll approximately 30 evaluable subjects in total. Patients with locally recurrent or metastatic breast cancer, with metastatic clear cell renal carcinoma, with locally advanced or metastatic non-small cell lung cancer are eligible to participate if they meet all inclusion criteria and none of the exclusion criteria.

Study Populations: Patients with locally recurrent or metastatic breast cancer, or with metastatic clear cell renal cell carcinoma or with locally advanced metastatic non-small cell lung cancer.

Primary Objectives: To describe the safety of ⁶⁸Ga-complex of a compound of Formula (I).

Secondary Objectives: To describe the biodistribution of ⁶⁸Ga-complex of a compound of Formula (I), to compare Formula (I) positron emission tomography/computed tomography (PET/CT) scans to standard of care anatomic imaging in detecting tumor lesions.

Primary Endpoints: Incidence of adverse events (AE) characterized overall and by type, frequency, seriousness, relationship to the study drug, timing, and severity, graded according to the National Cancer Institute (3NCI) Common Terminology Criteria for Adverse Events (CTCAE), Version 5.0.

Secondary Endpoints: Maximum standard uptake value (SUVₘₐₓ) of each tumor and SUVₘₑₐₙ of organs. Ratio of the tumor SUV over reference region SUV. Number and location of tumors identified by a ⁶⁸Ga-complex of a compound of Formula (I) PET/CT, and concordance rate between ⁶⁸Ga-complex of a compound of Formula (I) PET/CT and standard of care images.

Study Design: This study will enroll up to approximately 30 evaluable patients. Patients who receive study drug and complete all scheduled imaging procedures are considered evaluable.

There are three patient cohorts in the study: Cohort 1: patients locoregionally recurrent or metastatic breast cancer; Cohort 2: patients with metastatic clear cell renal cell carcinoma; Cohort 3. patients with locally advanced or metastatic non-small cell lung cancer. Patients are enrolled into the three cohorts in parallel. Each cohort will enroll ten evaluable patients. All patients will be evaluated for eligibility prior to enrollment (56-day screening period).

Each eligible patient enrolled will receive a single intravenous injection of a ⁶⁸Ga-complex of a compound of Formula (H) on Day 1 of the study at a dose of approximately 5 mCi (185 MBq). Initial patients (approximately 6) will be imaged with a dynamic PET scan in addition to a static whole-body PET/CT scan to establish optimal imaging time for subsequent patients who will undergo a single time-point PET/CT scan following injection of a ⁶⁸Ga-complex of a compound of Formula (I).

All patients must meet all the inclusion eligibility criteria and none of the exclusion eligibility criteria, as appropriate and provided below.

Inclusion Criteria: Locally recurrent or metastatic breast cancer (Cohort 1 patients); metastatic clear cell renal cell carcinoma (Cohort 2 patients); locally advanced or metastatic non-small cell lung cancer (Cohort 3 patients). Male or non-pregnant, non-lactating female subjects age ≥18 years. Subjects who are sexually active must agree to use adequate method(s) of effective contraception during their participation in the study. Eastern Cooperative Oncology Group (ECOG) Performance Status ≤2. Adequate hepatic function as defined by a) serum alanine aminotransarninase (ALT)/aspartate aminotransaminase (AST) 0.3× upper limit of normal (ULN) or ≤5×ULN if liver metastases are present or received prior mitotane therapy, and b) serum bilirubin −total ≤1.5×ULN (unless due to Gilbert's syndrome or hemolysis in which case total ≤3.0×ULN). Adequate renal function as measured by creatinine clearance calculated by the Cockcroft-Gault formula (≤60 mL/minute). Able to understand and willing to sign written informed consent.

Exclusion Criteria: Administered a radionuclide within a period of time corresponding to less than 10 physical half-lives of the radionuclide prior to study Day 1. Radiotherapy ≤14 days prior to study Day 1. Major surgery ≤21 days prior to study Day 1 or has not recovered from adverse effects of such procedure. History of cerebrovascular accident within 6 months or that resulted in ongoing neurologic instability. History of other previous or concurrent cancer that would interfere with the determination of safety. Any other condition that in the opinion of the Investigator would place the subject at an unacceptable risk or cause the subject to be unlikely to fully participate or comply with study procedures.

### Study Drug, Dose, and Mode of Administration

Study Drug: The study drug is a ⁶⁸Ga-complex of a compound of Formula (I). In some embodiments, the ⁶⁸Ga-complex is a ⁶⁸Ga-complex of a compound of Formula (I). In some embodiments, the ⁶⁸Ga-complex is a ⁶⁸Ga-complex of Compound 1. In some embodiments, the ⁶⁸Ga-complex is a ⁶⁸Ga-complex of Compound 6. In some embodiments, the ⁶⁸Ga-complex is a ⁶⁸Ga-complex of Compound 105. In some embodiments, the ⁶⁸Ga-complex is a ° ⁸Ga-complex of Compound 118. In some embodiments, the ⁶⁸Ga-complex is a ⁶⁸Ga-complex of Compound 193. In some embodiments, the ⁶⁸Ga-complex is a ⁶⁸Ga-complex of Compound 196. In some embodiments, the ⁶⁸Ga-complex is a ⁶⁸Ga-complex of Compound 269. In some embodiments, the ⁶⁸Ga-complex is a ⁶⁸Ga-complex of Compound 332. In some embodiments, the ⁶⁸Ga-complex is a ⁶⁸Ga-complex of Compound 366.

Dose: 5.0 mCi (±20%); Total carrier mass of the compound of Formula (I): not more than (NMT) 90 μg/dose.

### Mode of Administration: Intravenous

Duration of Participation: A 56-day screening window will be utilized where the subject will undergo study assessments to deem the subject eligible for the study. Once confirmed, subjects will receive the study drug, ⁶⁸Ga-complex of a compound of Formula (I), and PET/CT imaging on Day 1. The subjects will have a safety evaluation on Day 2 (+2 days).

Study Duration: The start of the study will be the date on which the first subject provides informed consent. The end of the study will be when the database is locked

The examples and embodiments described herein are for illustrative purposes only and various modifications or changes suggested to persons skilled in the art are to be included within the purview of this application and scope of the appended claims. Other embodiments are set forth in the following claims, along with the full scope of equivalents to which such claims are entitled.

What is claimed is:

1. A compound of Formula (I), or a pharmaceutically acceptable salt thereof: 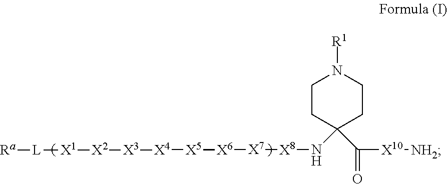
wherein:
R^{a} is a chelating moiety for a radionuclide or a radionuclide complex thereof;
L is an optional linker that is attached to any one of X¹, X², X³, X⁴, X⁵, X⁶, or X⁷; or
L is attached to X⁸ if X¹, X², X³, X⁴, X⁵, X⁶, and X⁷ are absent; and
R¹ is H 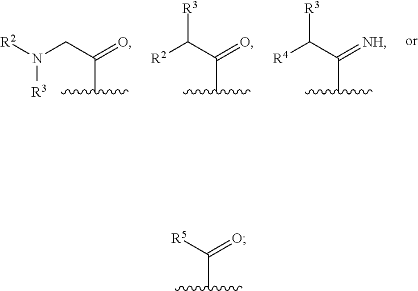
R² is C₁-C₈ alkyl, substituted or unsubstituted heteroalkyl, —(CHR⁶)ₙ-heterocycloalkyl, —(CHR⁶)ₙ-aryl, —(CHR⁶)ₙ-heteroaryl, —C(═O)—(CHR⁶)ₙ-aryl, or —C(═O)NH—(CHR⁶)ₙ-aryl; wherein C₁-C₈ alkyl is optionally substituted with R⁷, and wherein the heterocycloalkyl, aryl, or heteroaryl are each independently optionally substituted with R⁷, R⁸, R⁹, R¹⁰, and R¹¹;
R³ is H or C₁-C₄ alkyl;
R⁴ is H, C₁-C₄ alkyl, or R²;
R⁵ is substituted or unsubstituted alkyl, substituted or unsubstituted heteroalkyl, or —(CHR⁶)ₙ-aryl; wherein aryl is optionally substituted with R⁷, R⁸, R⁹, R¹⁰, and R¹¹;
each R⁶ is independently H, F, —CH₃, ˜NH₂, or —OH;
R⁷, R⁸, R⁹, and R¹⁰, R¹¹ are each independently selected from H, F, Cl, Br, I, —OH, —O—C₁—C₄ alkyl, —NH₂, —NHC₁-C₄ alkyl, —N(C₁-C₄ alkyl)₂, —CN, —CO₂H, —CO₂C₁-C₄ alkyl, —C₁-C₆ alkyl, —C₁-C₆ fluroroalkyl or —C₁-C₆ cycloalkyl;
n is 0, 1, 2, 3, 4, 5, or 6;
X¹ is absent, tyrosine (Tyr), glycine (Gly), sarcosine (Sar), alanine (Ala), aspartic acid (Asp), lysine (Lys), phenylalanine(Phe), 3-(3-pyridyl)alanine (3-Pal), threonine (Thr), methionine (Met), 4-iodophenylalanine (Phe(4-I)), N6-(4-(p-tolyl)butanoyl)-lysine, N6-(4-(4-iodophenyl)butanoyl)-lysine, or γ-glutamic acid (γ-Glu));
X² is absent, asparagine (Asn), glutamine (Gln), aspartic acid (Asp), glutamic acid (Glu), serine (Ser), histidine (His), alanine (Ala), sarcosine (Sar), tyrosine (Tyr), proline (Pro), hydroxyproline (Hyp), azetidine-2-carboxylic acid (Aze), 2,3,4,5-tetrahydroisoquinoline-3-carboxylic acid (Tic), phenylalanine (Phe), 3-(2-pyridyl)alanine (2-Pal), 3-(3-pyridyl)alanine (3-Pal), or 3-(4-pyridyl)alanine (4-Pal);
X³ is absent, tryptophan (Trp), serine (Ser), leucine (Leu), isoleucine (Ile), phenylalanine (Phe), 4-iodophenylalanine (Phe(4-I)), 3-(2-pyridyl)alanine (2-Pal), 3-(3-pyridyl)alanine (3-Pal), 3-(4-pyridyl)alanine (4-Pal), 2-amino-3-(naphthalen-2-yl)propanoic acid (H-2-NAL-OH), lysine (Lys), asparagine (Asn), glutamine (Gln), aspartic acid (Asp), glutamic acid (Glu), arginine (Arg), methyl arginine (Arg(Me)), norarginine (AGBA), methyl norarginine (AGBA(Me)), homoarginine (Harg), methyl homoarginine (Harg(Me)), citrulline (Cit), methyl citrulline (Cit(Me)), canavanine, methyl-canavanine, glycine (Gly), alanine (Ala), sarcosine (Sar), tyrosine (Tyr), cyclohexylalanine (Cha), 3-(1-naphthyl)alanine((α-Nal), 3-(2-naphthyl)alanine (β-Nal), threonine (Thr), proline (Pro), hydroxyproline (Hyp), tetrahydroisoquinoline-3-carboxylic acid (Tic), O-phospho-serine (SOP), 2-amino-1-(2H-tetrazol-5-yl)butanoic acid, β-glutamic acid, 8-aminoquinoline-3-carboxylic acid, biphenylalanine (Bip), 4-benzoylphenylalanine (Bpa), or 3-(9-anthryl)-alanine (H-Ala(9-Anth)-OH or AAP);
X⁴ is absent, asparagine (Asn), glutamine (Gln), aspartic acid (Asp), glutamic acid (Glu), tryptophan (Trp), glycine (Gly), tyrosine (Tyr), alanine (Ala), sarcosine (Sar), or arginine (Arg);
X is absent, serine (Ser), threonine (Thr), lysine (Lys), asparagine (Asn), glutamine (Gln), aspartic acid (Asp), glutamic acid (Glu), glycine (Gly), alanine (Ala), or sarcosine (Sar);
X⁶ is absent, phenylalanine (Phe), alpha-methylphenylalanine (α-Me-Phe), N-methylphenylalanine (N-Me-Phe), 2-fluorophenylalanine (2-F-Phe), 3-fluorophenylalanine (3-F-Phe), 4-fluorophenylalanine (4-F-Phe), 4-iodophenylalanine (Phe(4-I)), 2-amino-2-indancarboxylic acid(Aic), biphenylalanine (Bip), (β-(2-thienyl)-Ala), tryptophan (Trp), 2-aminotetralin-2-carboxylic acid (Atc), 3β-(2-thienyl)-alanine, 3-(4-pyridyl)alanine (4-Pal), cyclohexylalanine (Cha), or tyrosine (Tyr);
X⁷ is absent, glycine (Gly), aza-glycine (aza-Gly), alanine (Ala), N-methylglycine (Sar), or 1-aminocyclopropane-1-carboxylic acid (ACC);
X⁸ is leucine (Leu), norvaline (Nva), valine (Val), isoleucine (lie), homoalanine (Hala), tryptophan (Trp), phenylalanine (Phe), or phenylglycine (Phg);
or —X⁷—X⁸— is 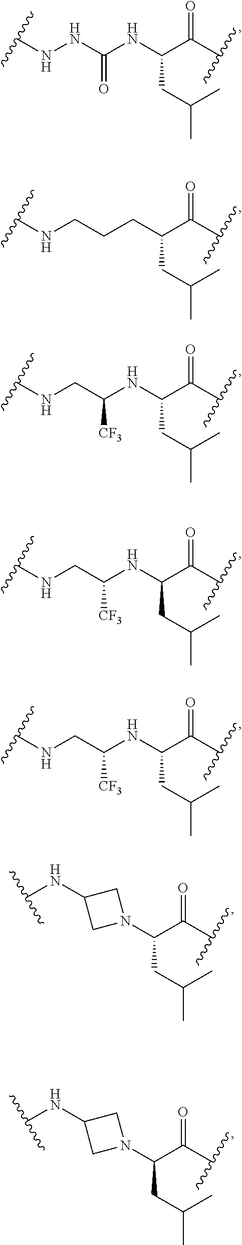 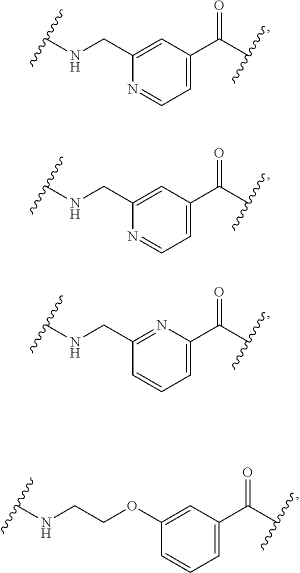 [SMILES: *NCc1cc(C(*)=O)ccn1 ; *NCc1cc(C(*)=O)ccn1 ; *NCc1cccc(C(*)=O)n1 ; *NCCOc1cccc(C(*)=O)c1] 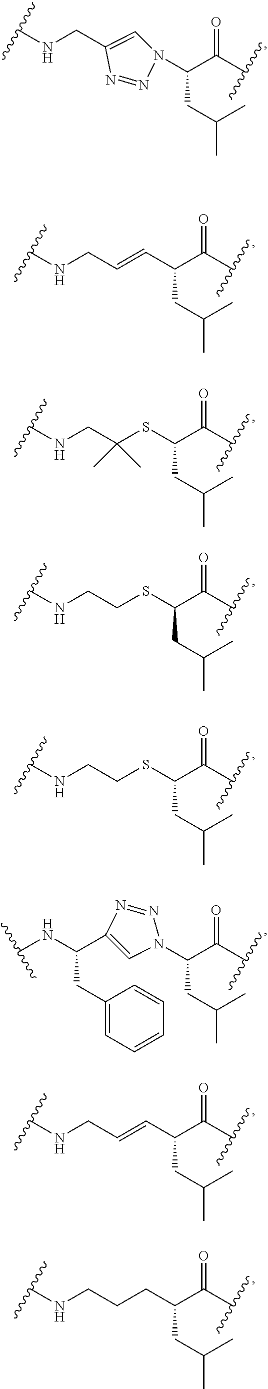 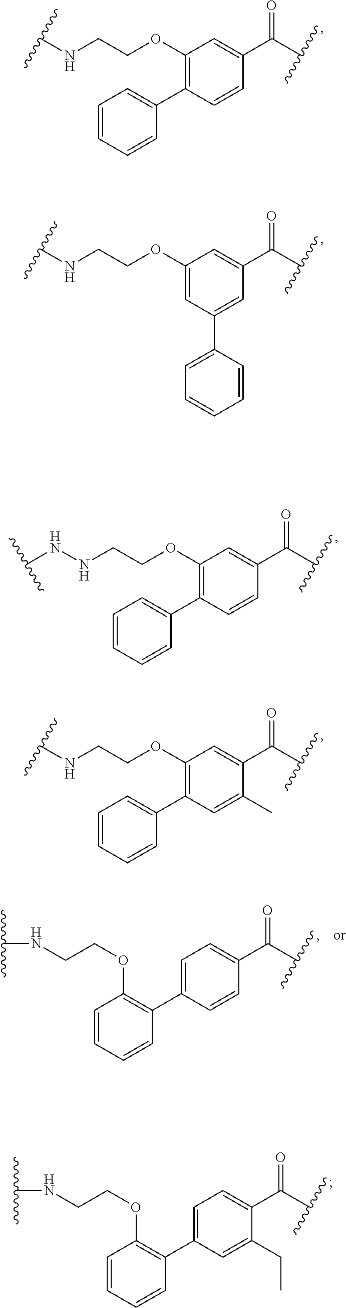 [SMILES: *NCCOc1cc(C(*)=O)ccc1-c1ccccc1 ; *NCCOc1cc(C(*)=O)cc(-c2ccccc2)c1 ; *NNCCOc1cc(C(*)=O)ccc1-c1ccccc1 ; *NCCOc1cc(C(*)=O)c(C)cc1-c1ccccc1 ; *NCCOc1ccccc1-c1ccc(C(*)=O)cc1 ; *NCCOc1ccccc1-c1ccc(C(*)=O)c(CC)c1]
or —X⁶—X⁷—X⁸— is 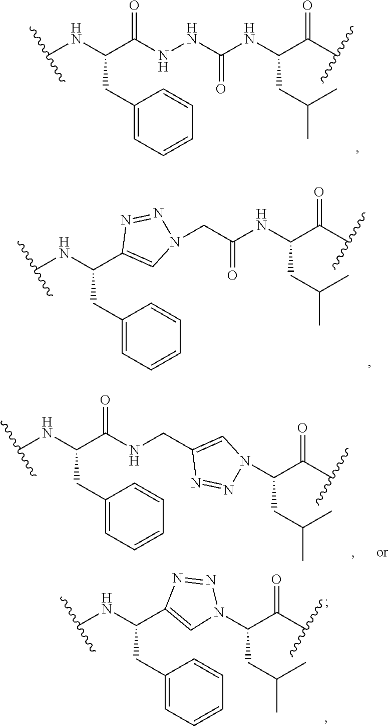
X¹⁰ is tryptophan (Trp), 1-methyltryptophan (1MT), tyrosine (Tyr), phenylalanine (Phe), 4-cyano phenylalanine (Phe(4-CN)), 3-(4-pyridyl)alanine (4-Pal), leucine (Leu), phenylglycine (Phg), cyclohexylalanine (Cha), 3-(I-naphthyl)alanine (α-Na), 3-(2-naphthyl)-alanine (β-Nal), histidine (His), or 3-nitro-tyrosine (Tyr(3-NO₂)),
wherein the N-terminal amino acid or the compound of Formula (I) is optionally substituted with —C(═O)—C₁-C₂₀ alkyl, —C(═O)—(CH₂CH₂O)_{y}—CH₂CH₂—R¹⁵, C₁-C₂₀ alkyl, N-hexadecanoyl-Glu, C₄-C₂₀ polyethylene glycol, —R¹⁶, —C(═O)—(CH₂C₂O)ₓ—CH₃, —C(O)—CH₂CH₂O)ₓ—H, —C(═O)CH₂CH₂CH(COOH)—R¹⁵, —C(═O)—(CH₂)₂R¹⁹, or —C(═O)CH₂NHCH₂R¹⁹;
R¹⁵ is selected from —OR¹⁶, —N(R¹⁶)₂, —C(═O)OR¹⁶, or —C(═O)N(R¹⁶)₂;
each R¹⁶ is independently H, —C₁-C₆ alkyl, —C(═O)—(CH₂)ᵥR¹⁹, or —C(═O)CH₂NHCH₂R¹⁹;
R¹⁹ is 4-iodophenylene, 4-methylphenylene, or 3-fluoro-4-methylphenylene;
y is 0, 1, 2, 3, 4, 5, 6, 7, 8, 9, or 10;
x is an integer from 1 and 25; and
v is 1, 2, 3, or 4;
wherein any free —NH— of a peptide bond is optionally independently substituted with —CH₃ or —CH₂CH₃; and
wherein any alpha position of an amino acid is optionally independently substituted with —CH₃ or —CH₂CH₃.

2. The compound of claim 1, or a pharmaceutically acceptable salt thereof, wherein: 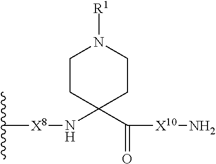
is 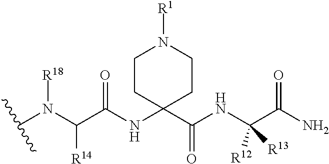
wherein,
R¹⁸ is H or —CH₃;
R¹² is 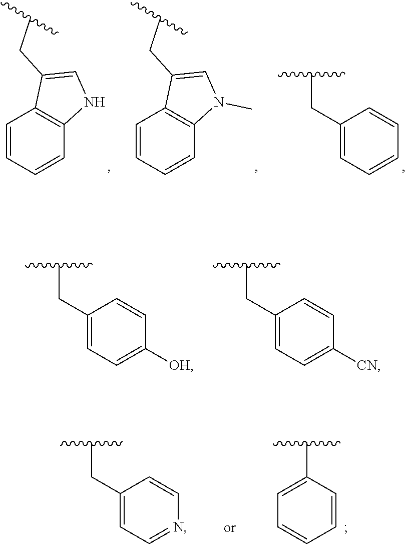
R¹³ is H or —CH₃; and
R¹⁴ is

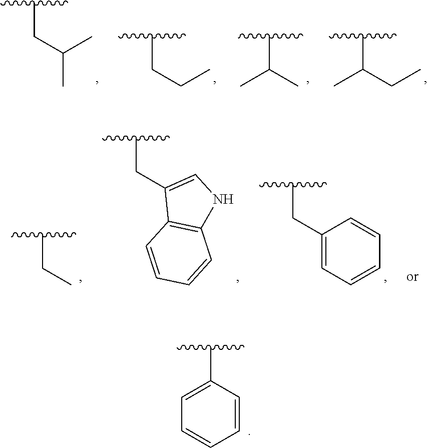

3. The compound of claim 1, or a pharmaceutically acceptable salt thereof,
wherein:
X⁶ is 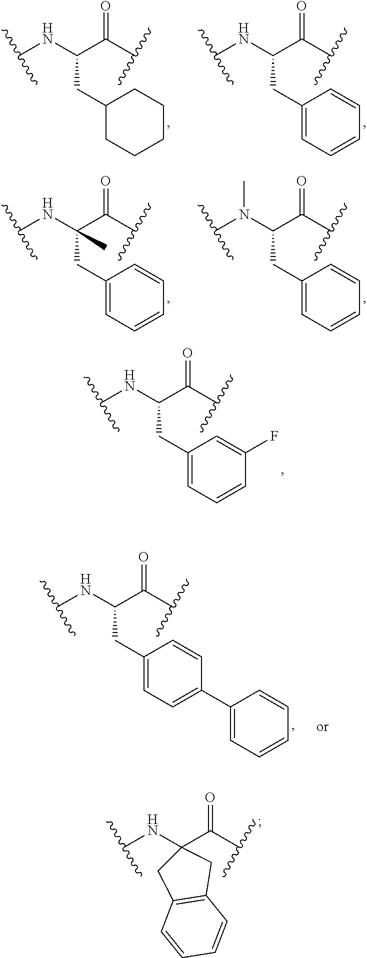
X⁷ is 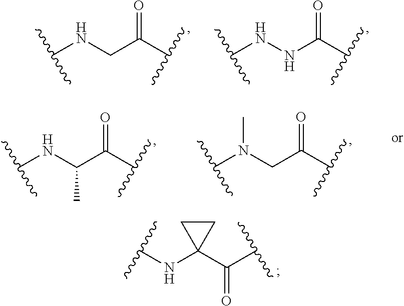
and X⁸ is

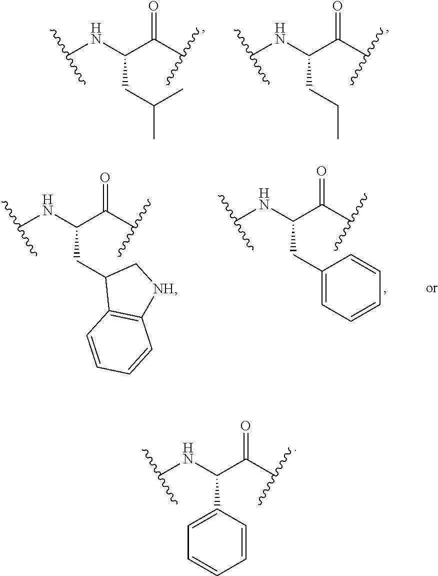

4. The compound of claim 1, or a pharmaceutically acceptable salt thereof, wherein:
X⁶ is 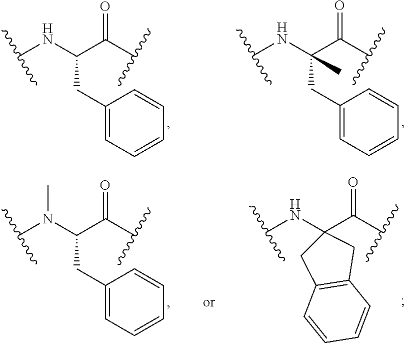
and X⁷—X⁸ is

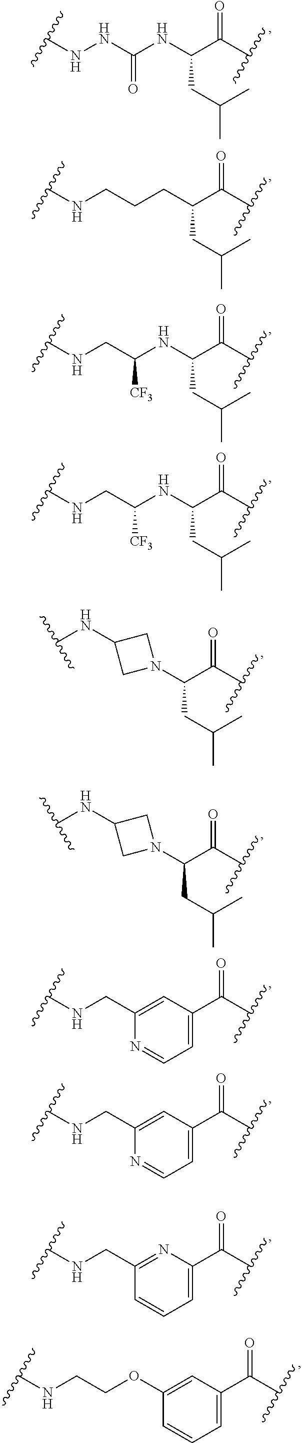

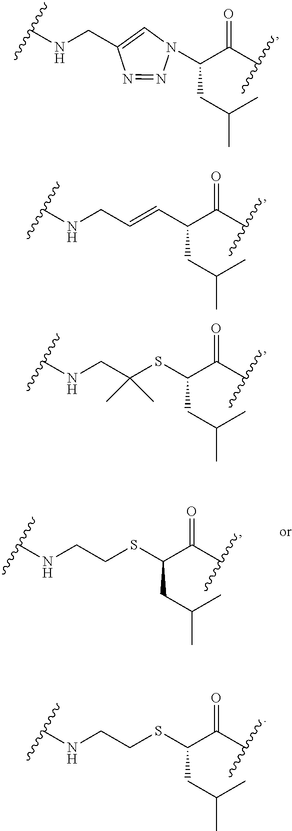

5. The compound of claim 1, or a pharmaceutically acceptable salt thereof, wherein: 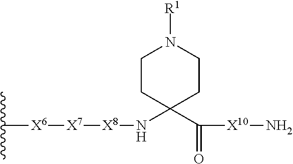
is

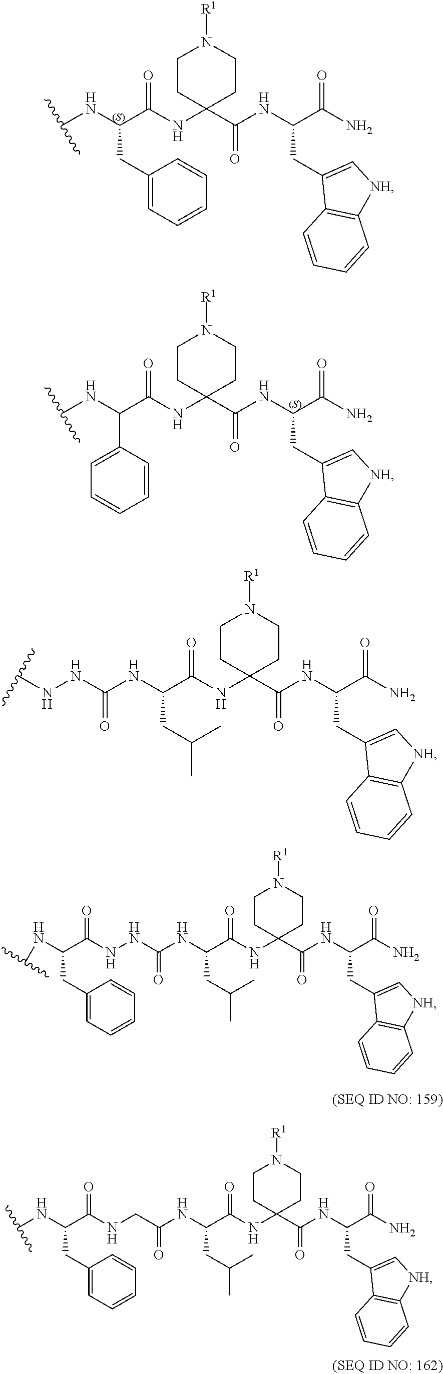

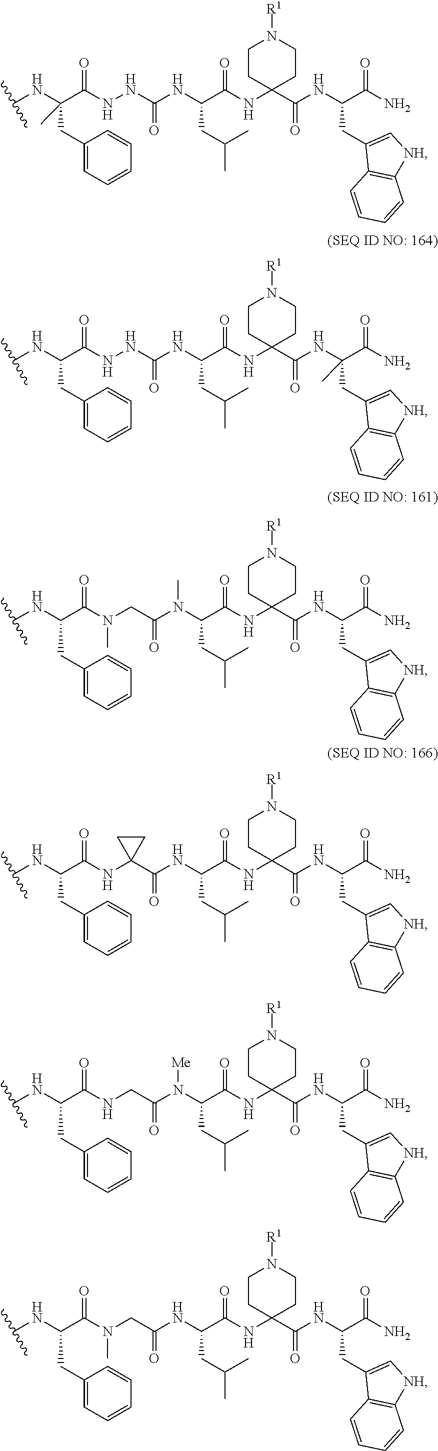

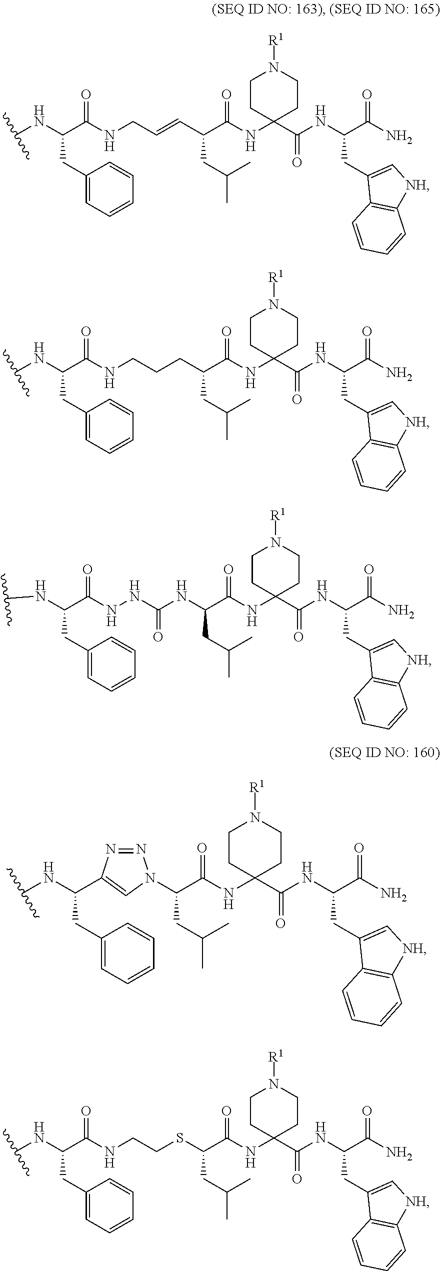

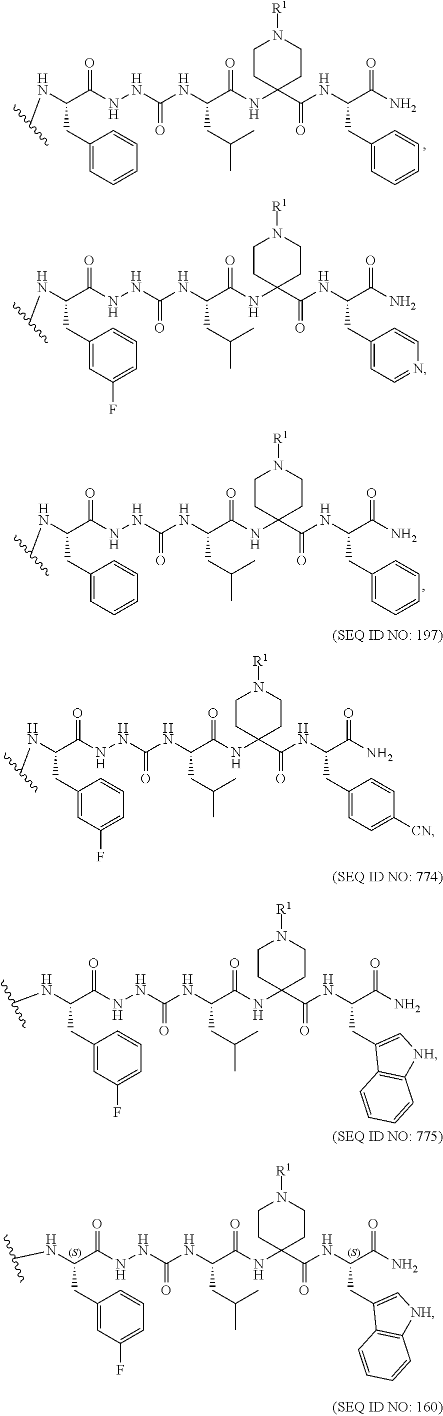

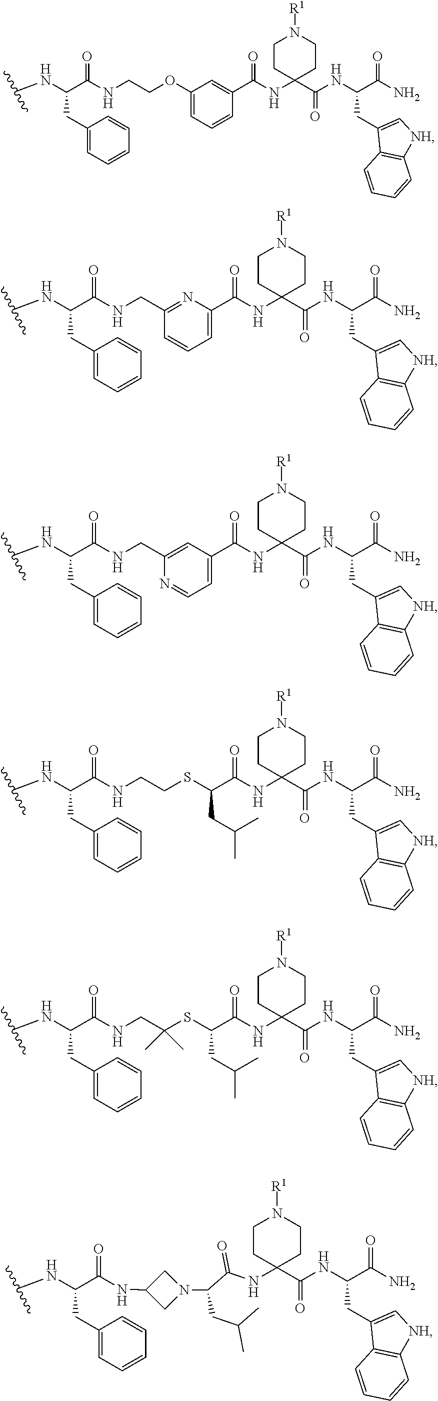

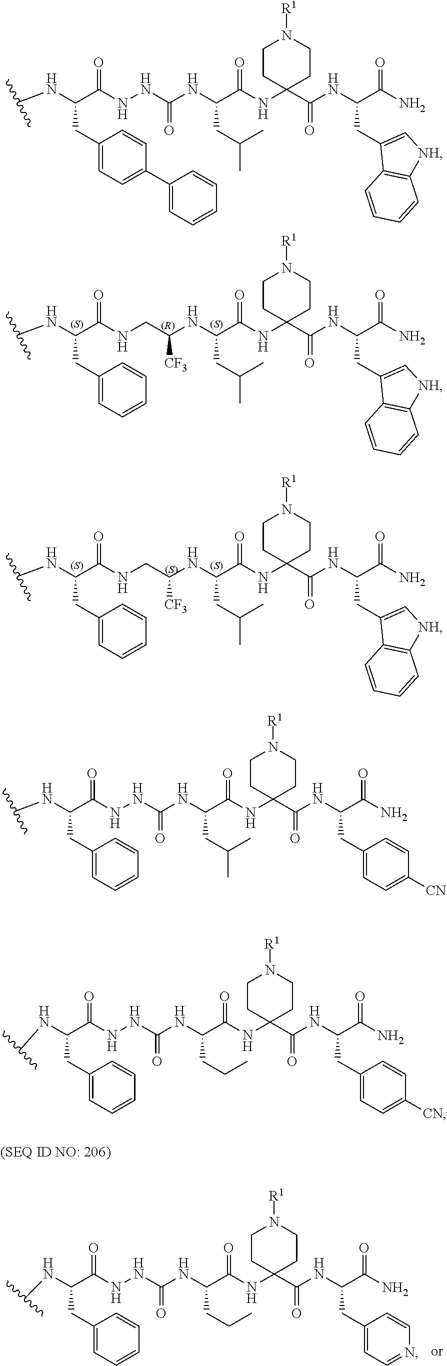

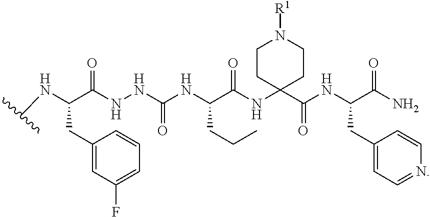

6. The compound of claim 1, or a pharmaceutically acceptable salt thereof, wherein: X¹⁰ is tryptophan (Trp), 1-methyltryptophan (1MT), tyrosine (Tyr), phenylalanine (Phe), 4-cyano phenylalanine(Phe(4-CN)), 3-(4-pyridyl)alanine (4-Pal), or leucine (Leu).

7. The compound of claim 1, or a pharmaceutically acceptable salt thereof, wherein R¹ is H,

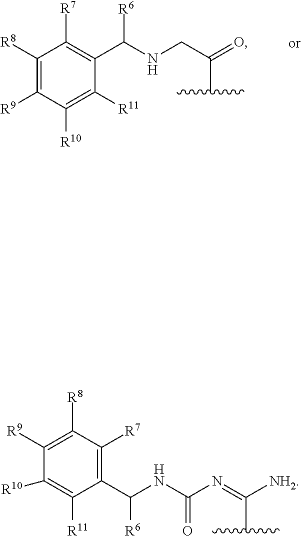

8. The compound of claim 1, or a pharmaceutically acceptable salt thereof, wherein:
R² is

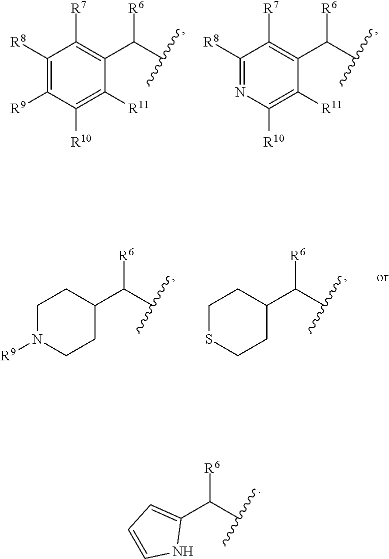

9. The compound of claim 1, or a pharmaceutically acceptable salt thereof, wherein:
R⁷, R⁸, R⁹, R¹⁰, and R¹¹ are independently selected from H, F, Cl, Br, I, —OH, —OCH₃, —OCH₂CH₃, —NH₂, —NHCH₃, —N(CH₃)₂, —CN, —CO₂H, —CO₂CH₃, —CO₂CH₂CH₃, —CH₃, —CH₂CH₃, —CH(CH₃)₂, —(CH₃)₃, —CF₃, —CH₂F, —CH₂F, or cyclopropyl.

10. The compound of claim 1, or a pharmaceutically acceptable salt thereof, wherein:
R¹ is

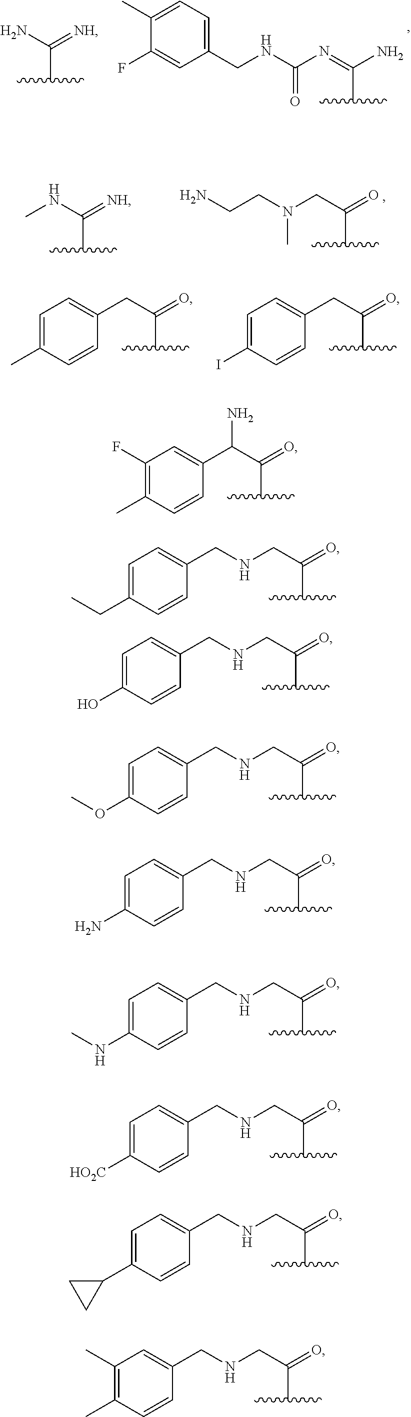

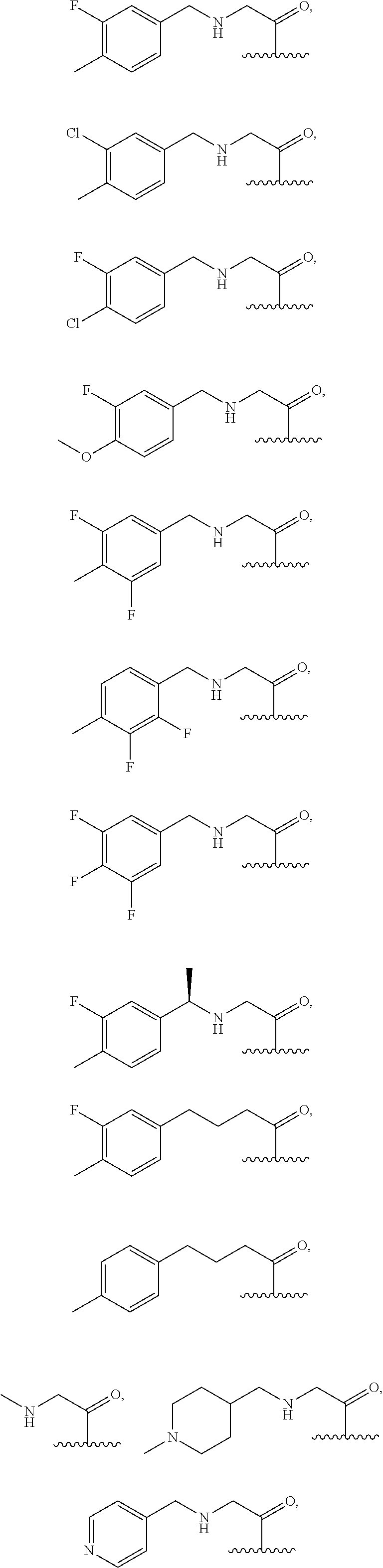

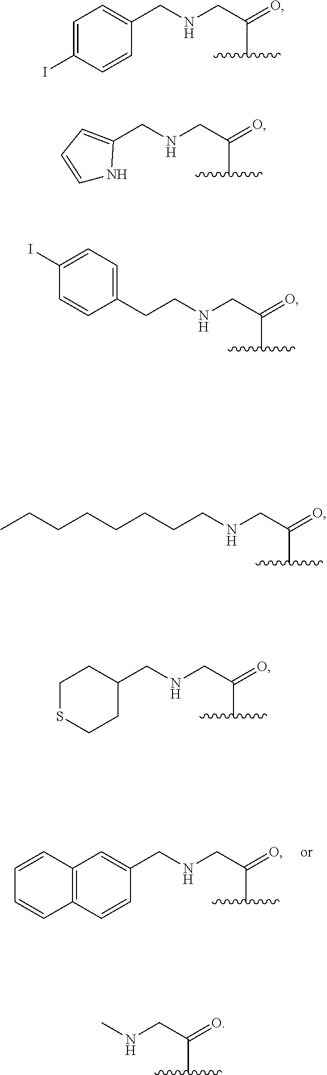

11. The compound of claim 1, or a pharmaceutically acceptable salt thereof, wherein:
R¹ is

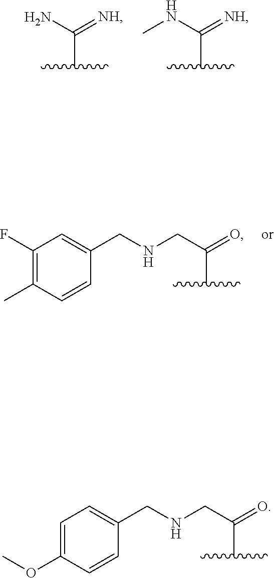

12. The compound of claim 1, or a pharmaceutically acceptable salt thereof, wherein:
X¹ is absent, Tyr, Asp, Lys, 3-Pal, Sar, or Phe;
X² is absent, Asn, Gin, Asp, Glu, Ser, His, Ala, Sar, Pro, Hyp, Aze, Tic, Phe, or 4-Pal;
X³ is Trp, Ser, Ile, Phe, 4-Pal, Lys, Asn, Gin, Asp, Glu, Arg, Arg(Me), Gly, Ala, Sar, Tyr, Cha, β-Nal, Hyp, Thr, Bip, Bpa, or AAP;
X⁴ is Asn, Gln, Asp, Glu, Trp, Gly, Ala, or Sat; and
X⁵ is absent, Thr, Ser, Gly, or Ala.

13. The compound of claim 1, or a pharmaceutically acceptable salt thereof, wherein:
X¹ is tyrosine (Tyr), glycine (Gly), or 3-(3-pyridyl)alanine (3-Pal));
X² is absent, asparagine (Asn), glutamine (Gln), serine (Ser), D-histidine (His), tyrosine (Tyr), or phenylalanine (Phe);
X³ is tryptophan (Trp), isoleucine (Ile), 3-(4-pyridyl)alanine (4-Pal), 2-amino-3-(naphthalen-2-yl)propanoic acid (H-2-NAL-OH), lysine (Lys), aspartic acid (Asp), glutamic acid (Glu), glycine (Gly), alanine (Ala), cyclohexylalanine (Cha), (Hyp), biphenylalanine (Bip); 4-benzoylphenylalanine (Bpa), or 3-(9-anthryl)-alanine (AAP);
X⁴ is asparagine (Asn), or glutamine (Gln); and
X⁵ is serine (Ser), threonine (Thr), glycine (Gly), or alanine (Ala).

14. The compound of claim 1, or a pharmaceutically acceptable salt thereof, wherein 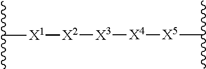
is absent,

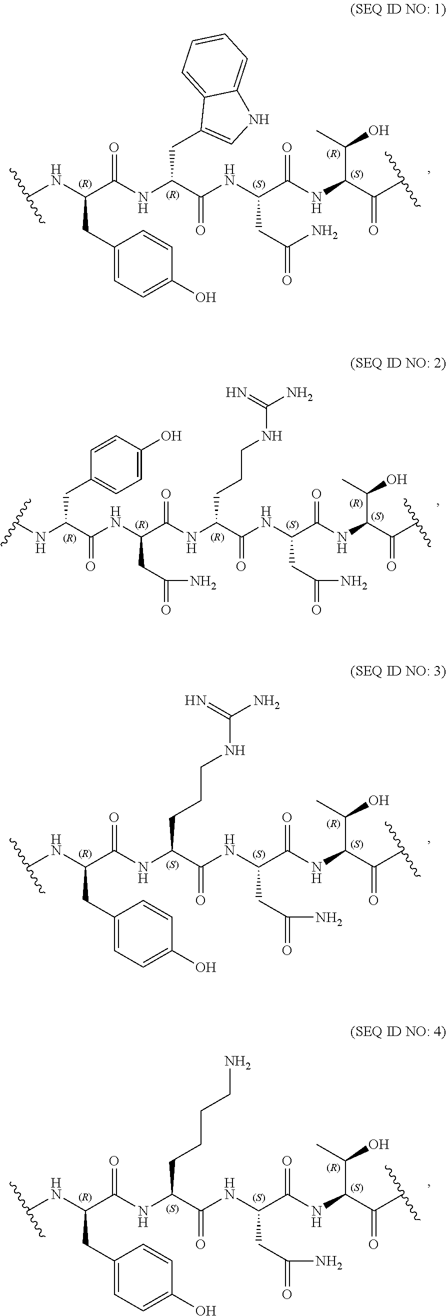

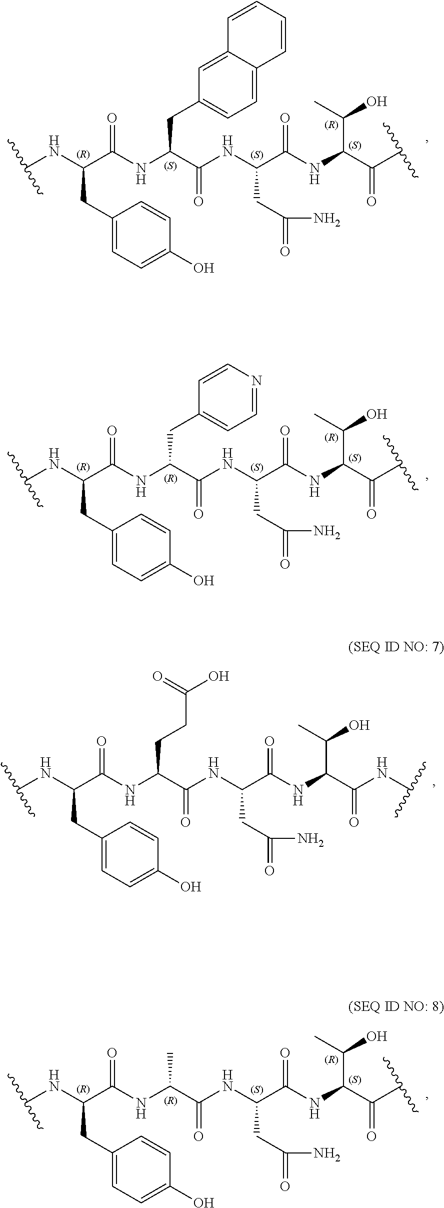

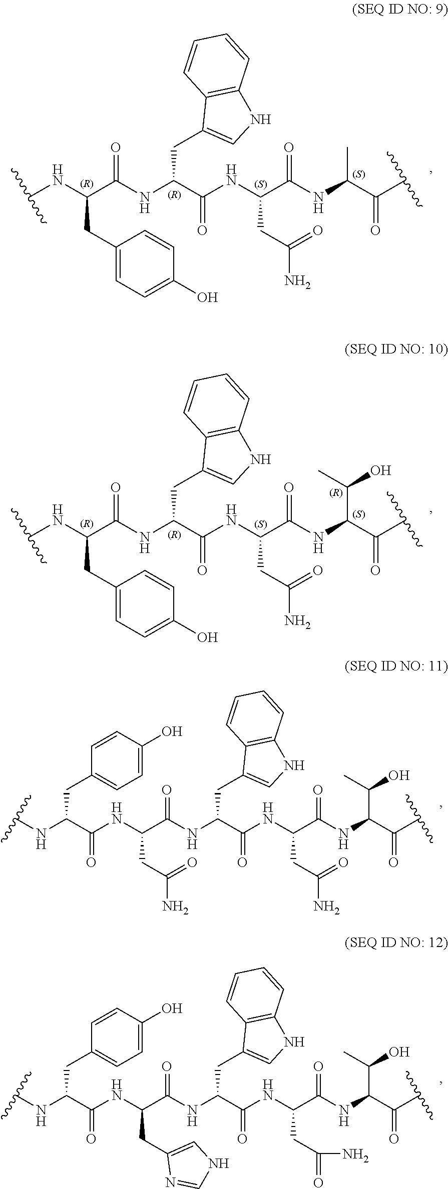

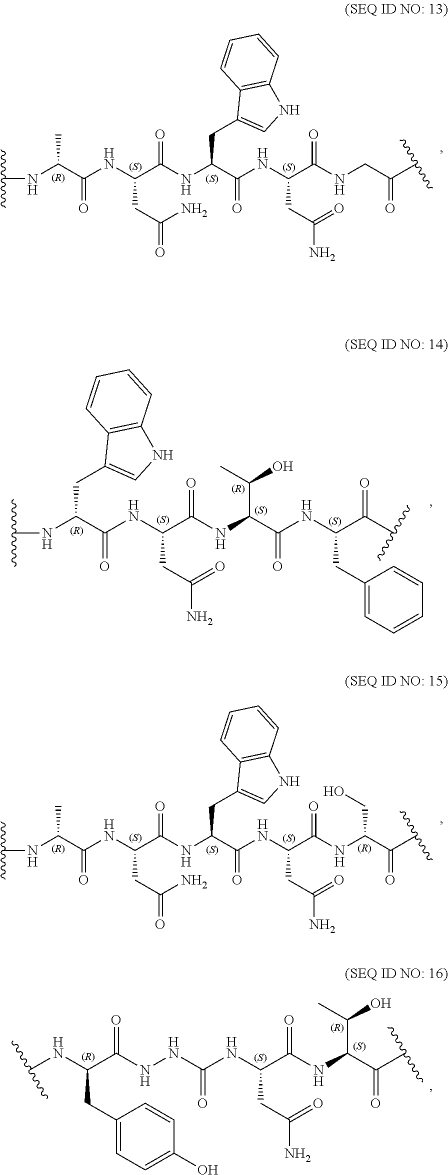

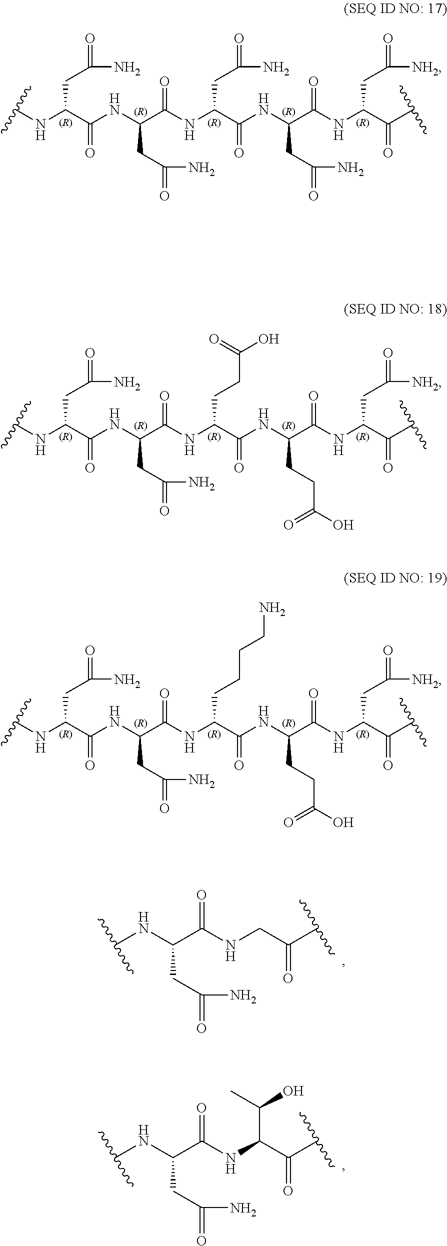

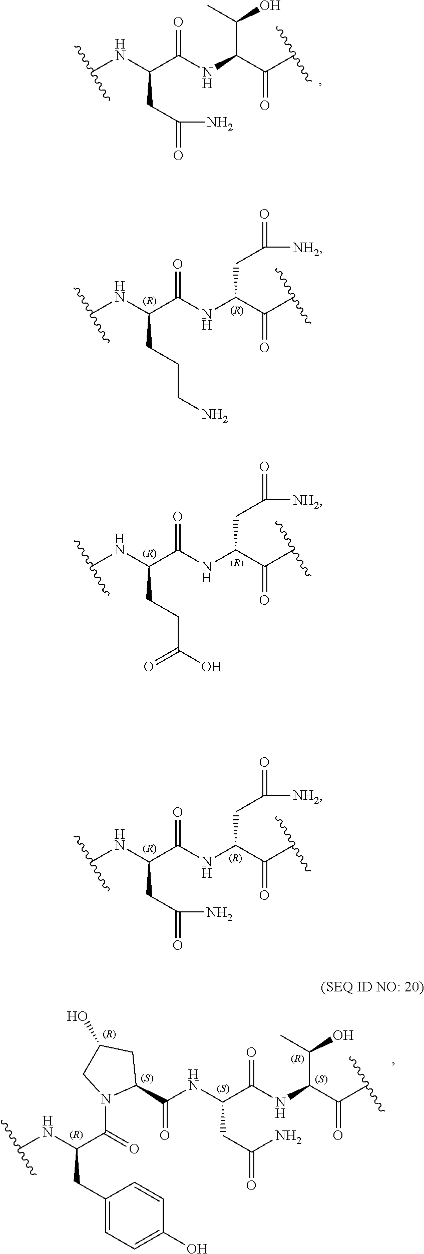

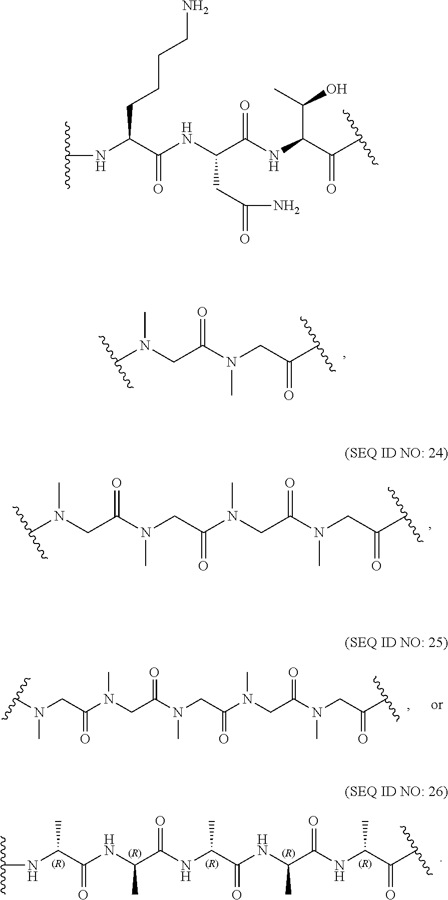

15. The compound of claim 1, or a pharmaceutically acceptable salt thereof, wherein: 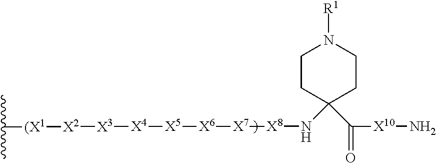
is

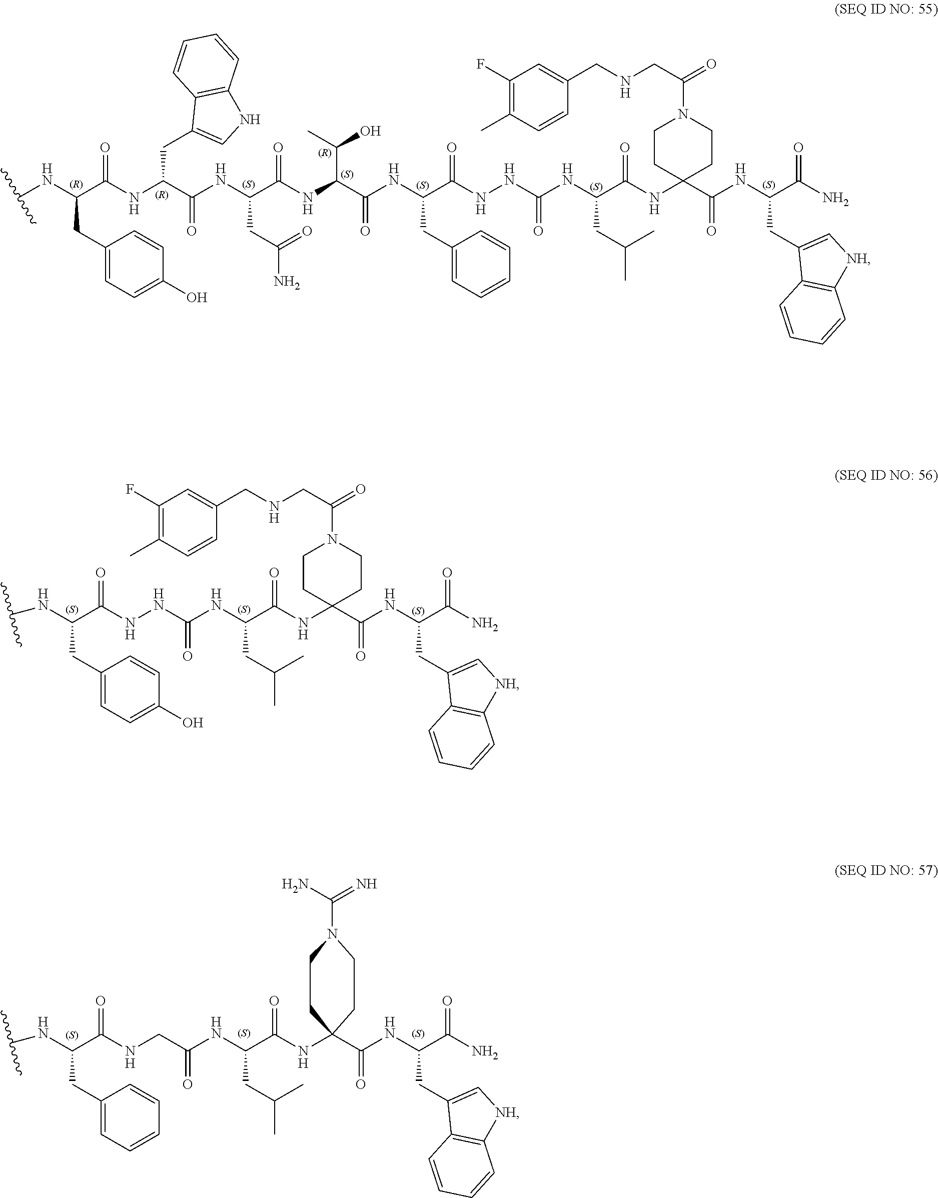

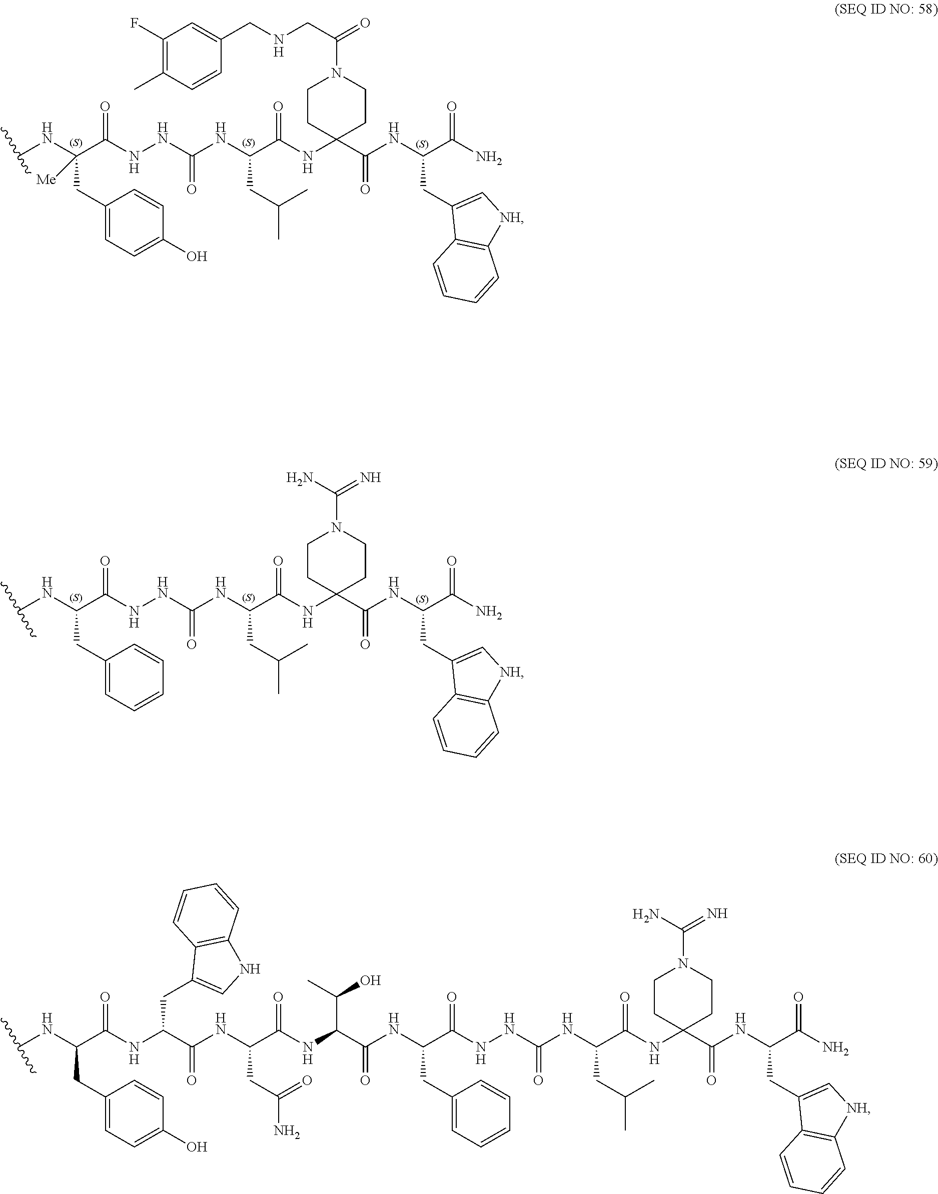

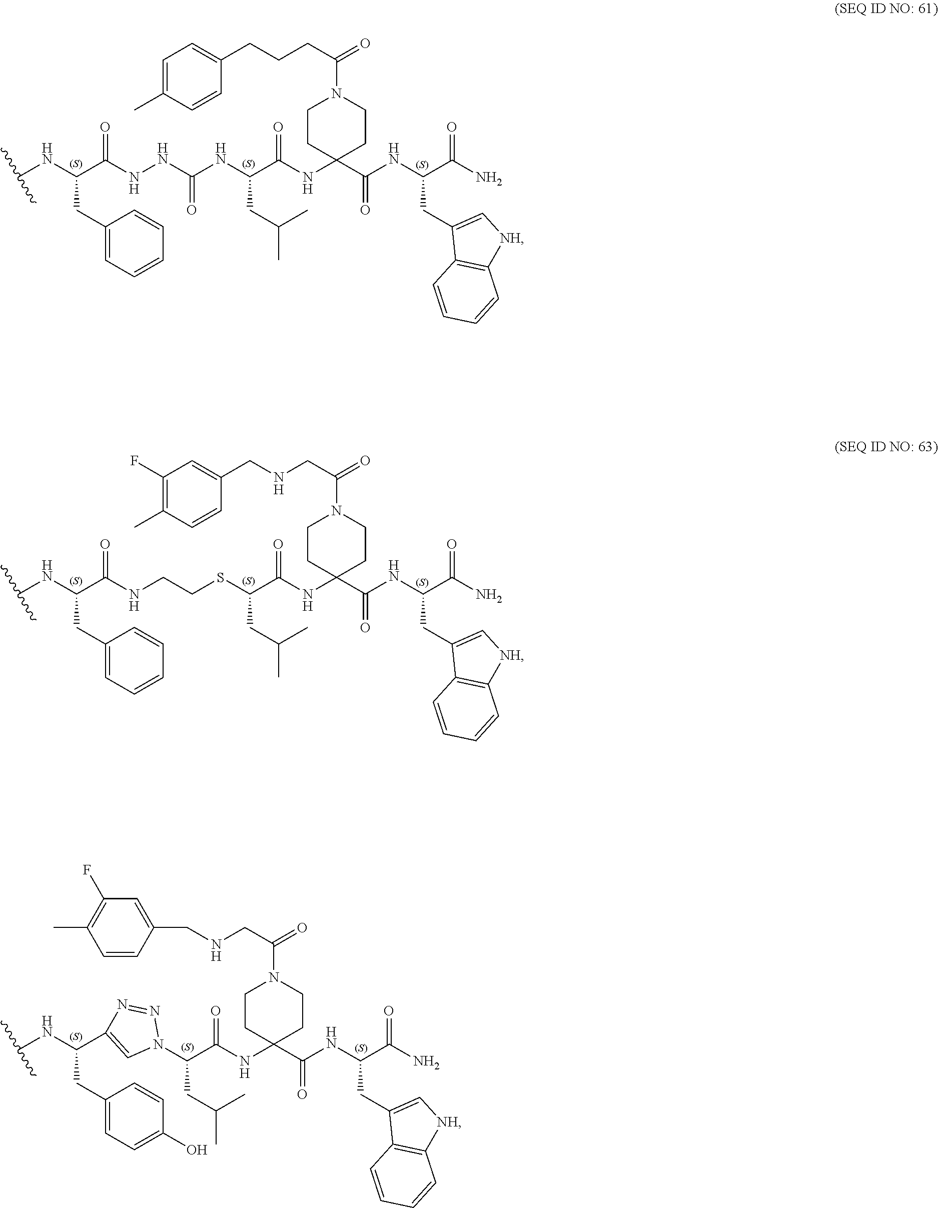

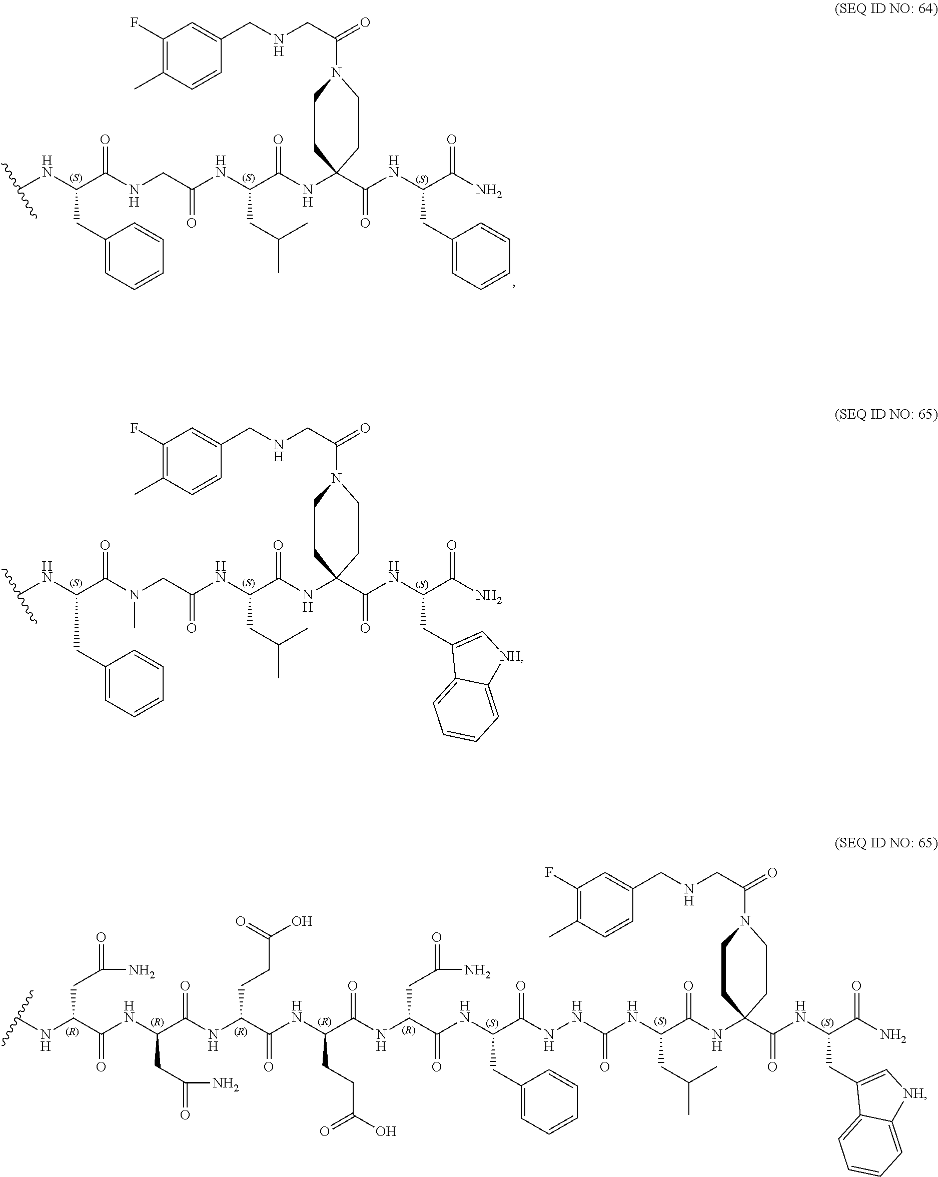

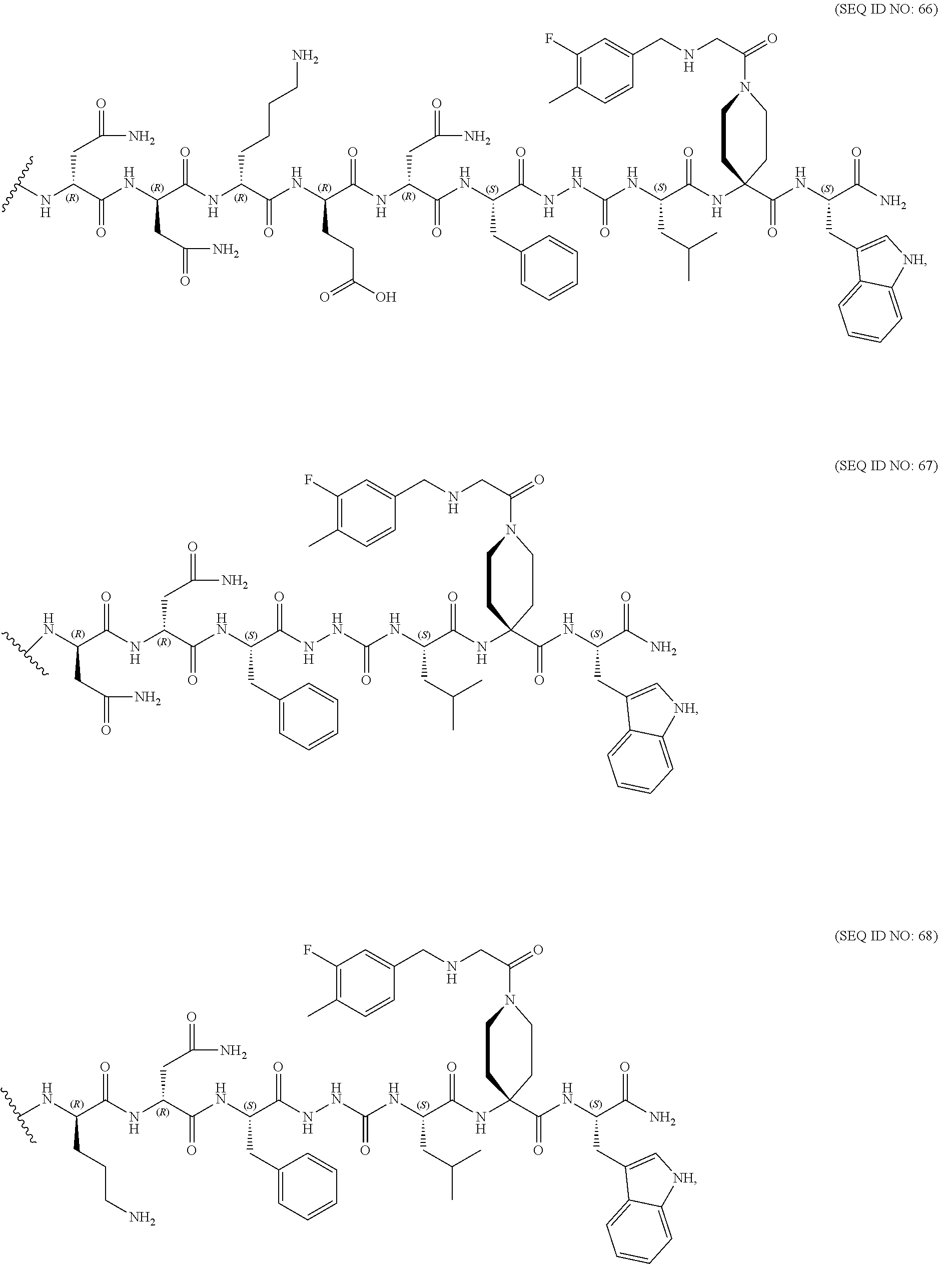

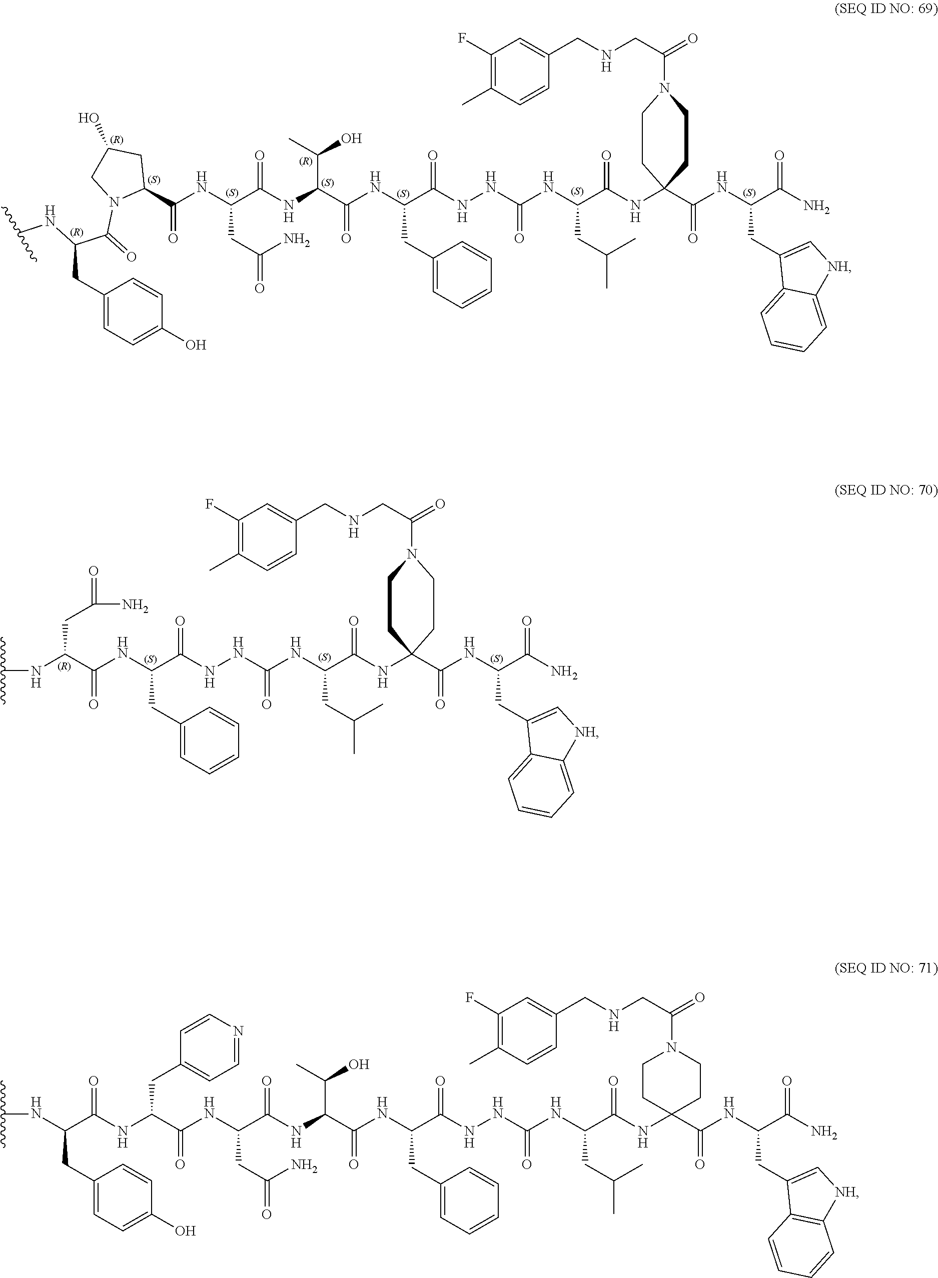

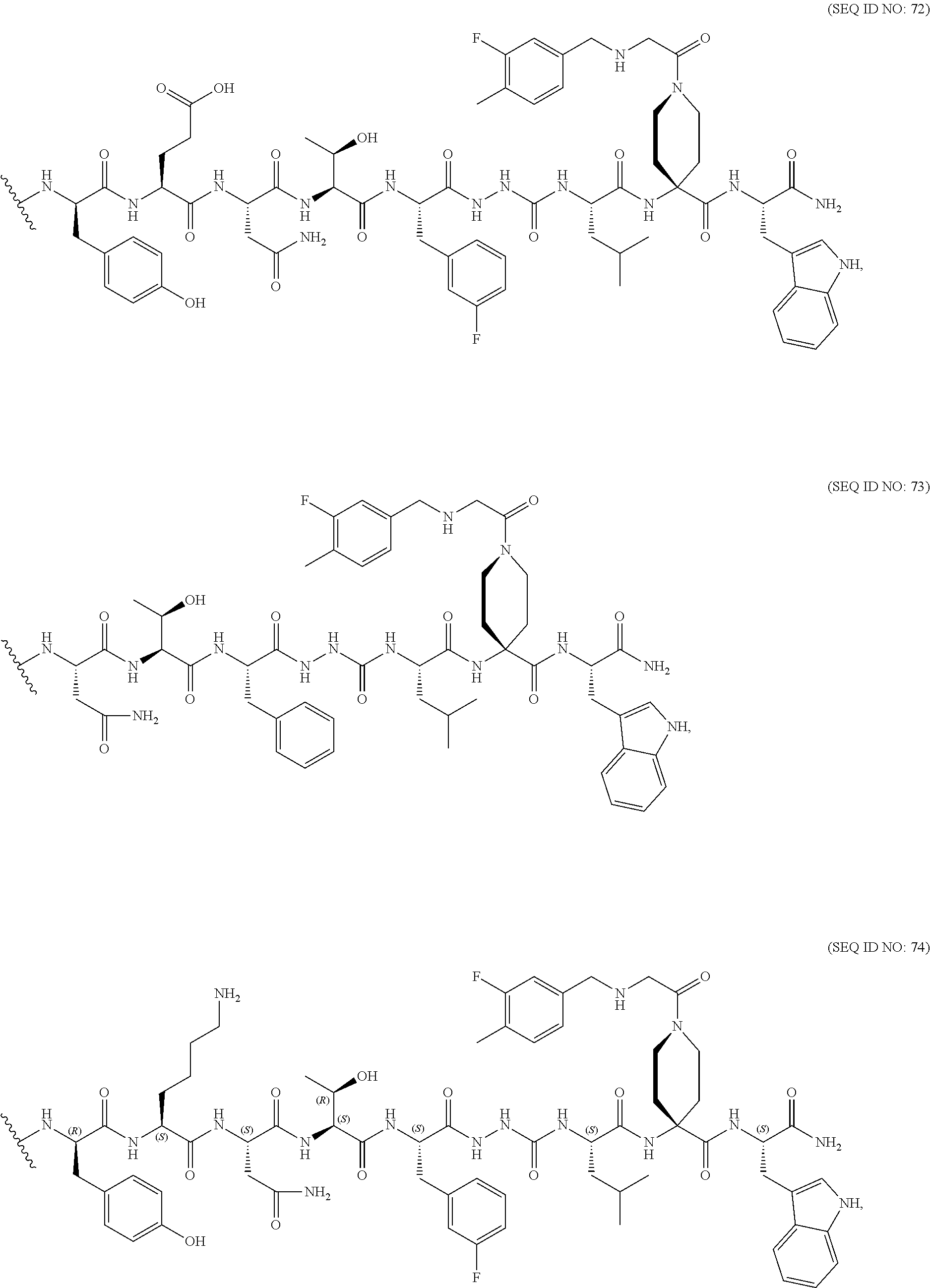

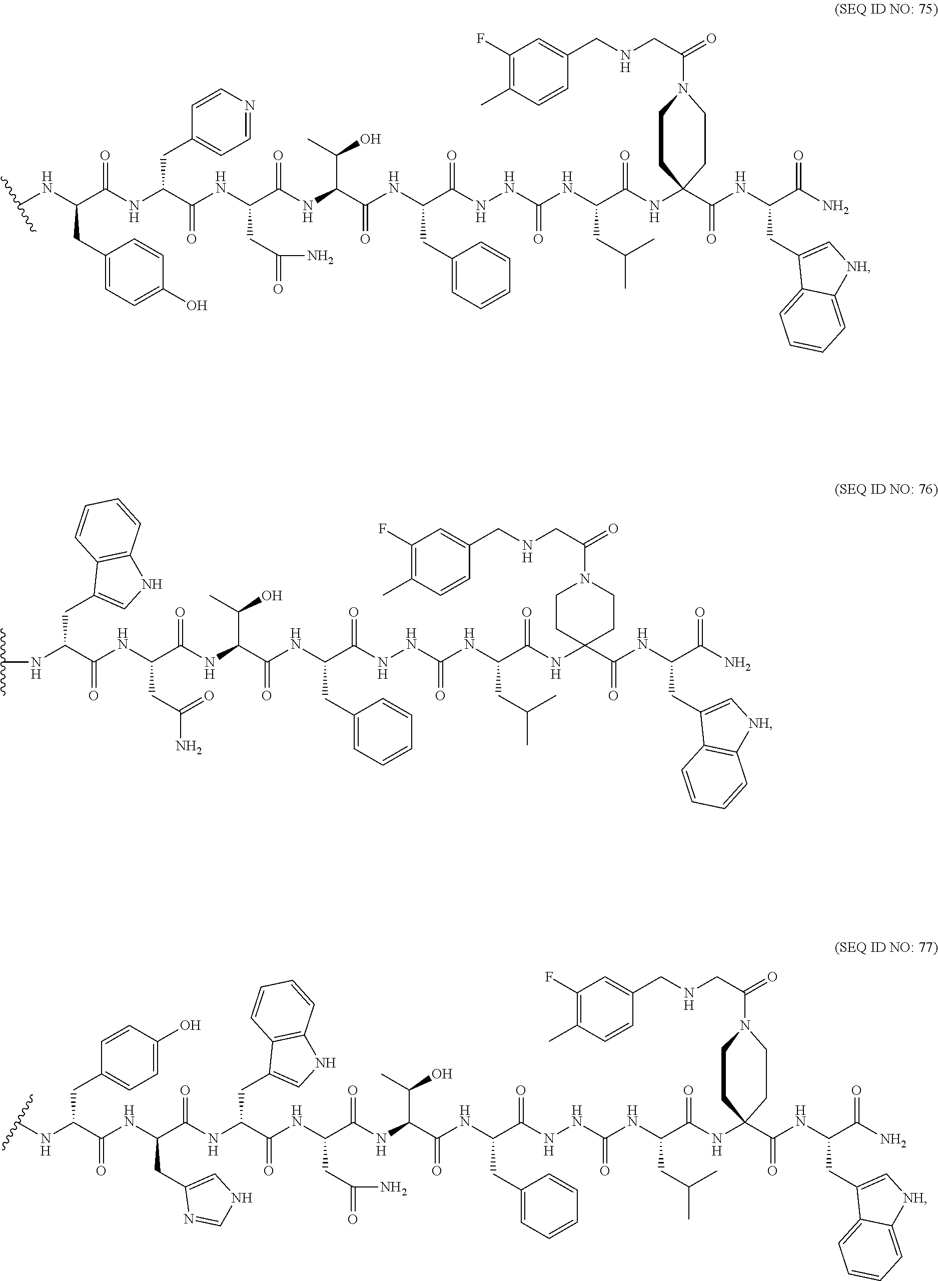

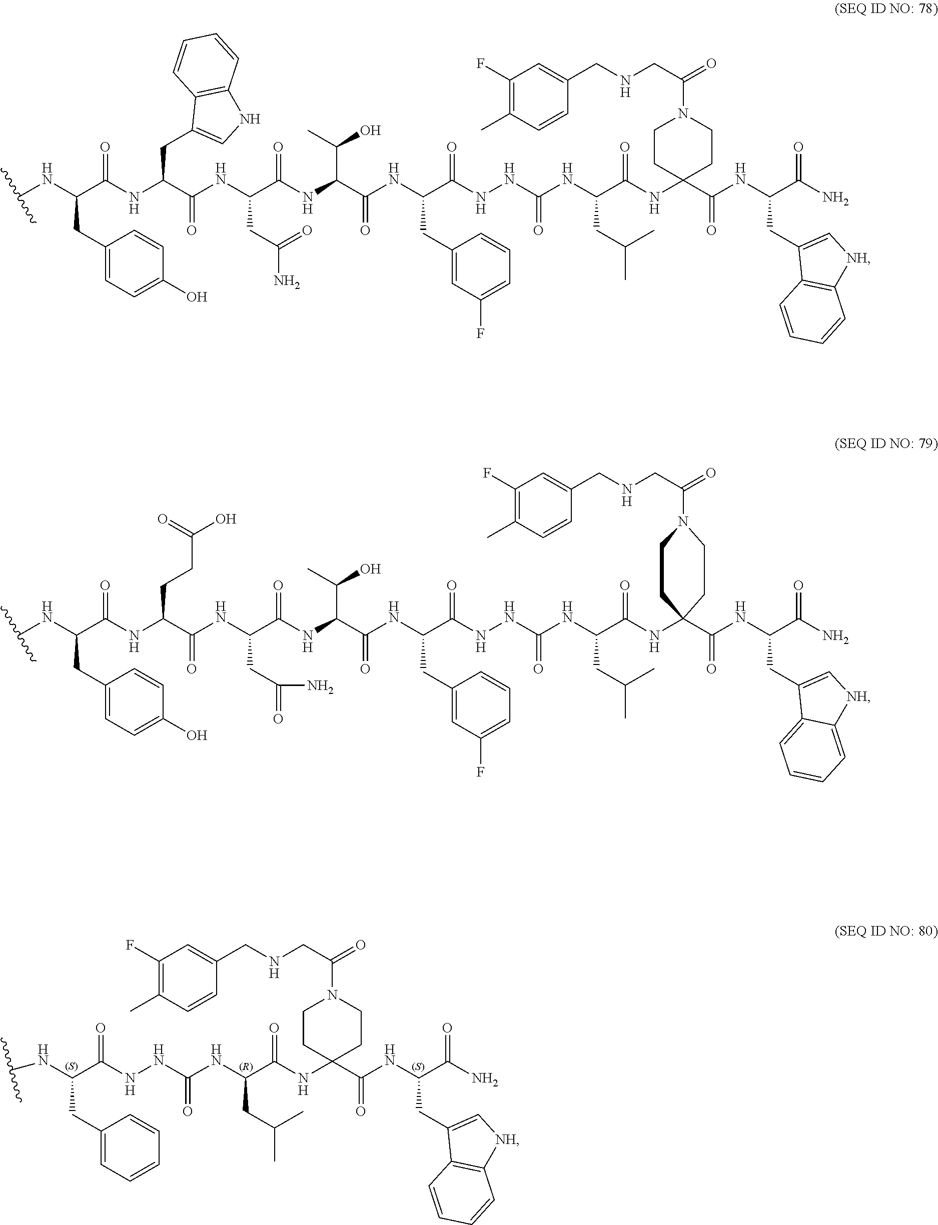

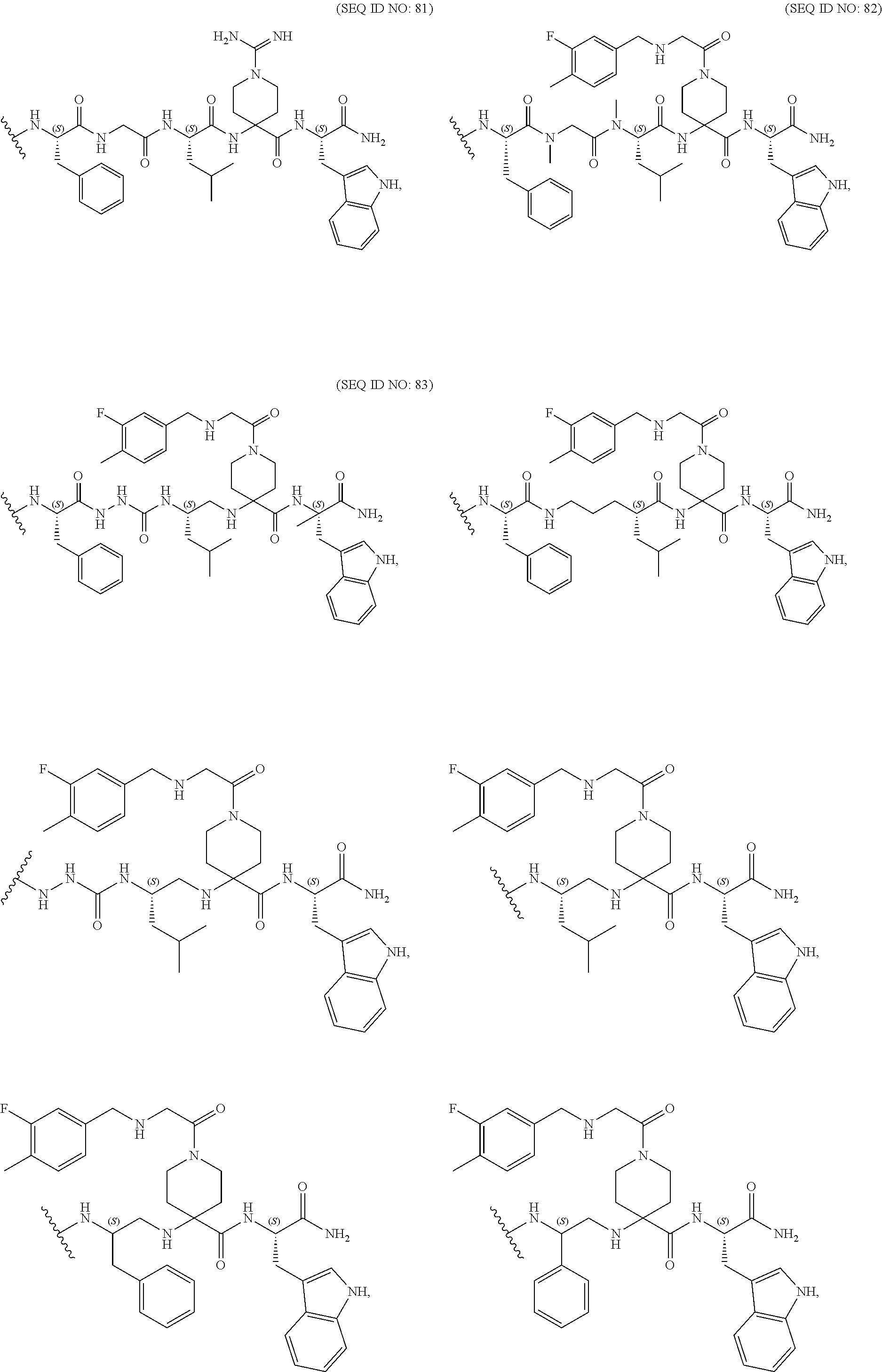

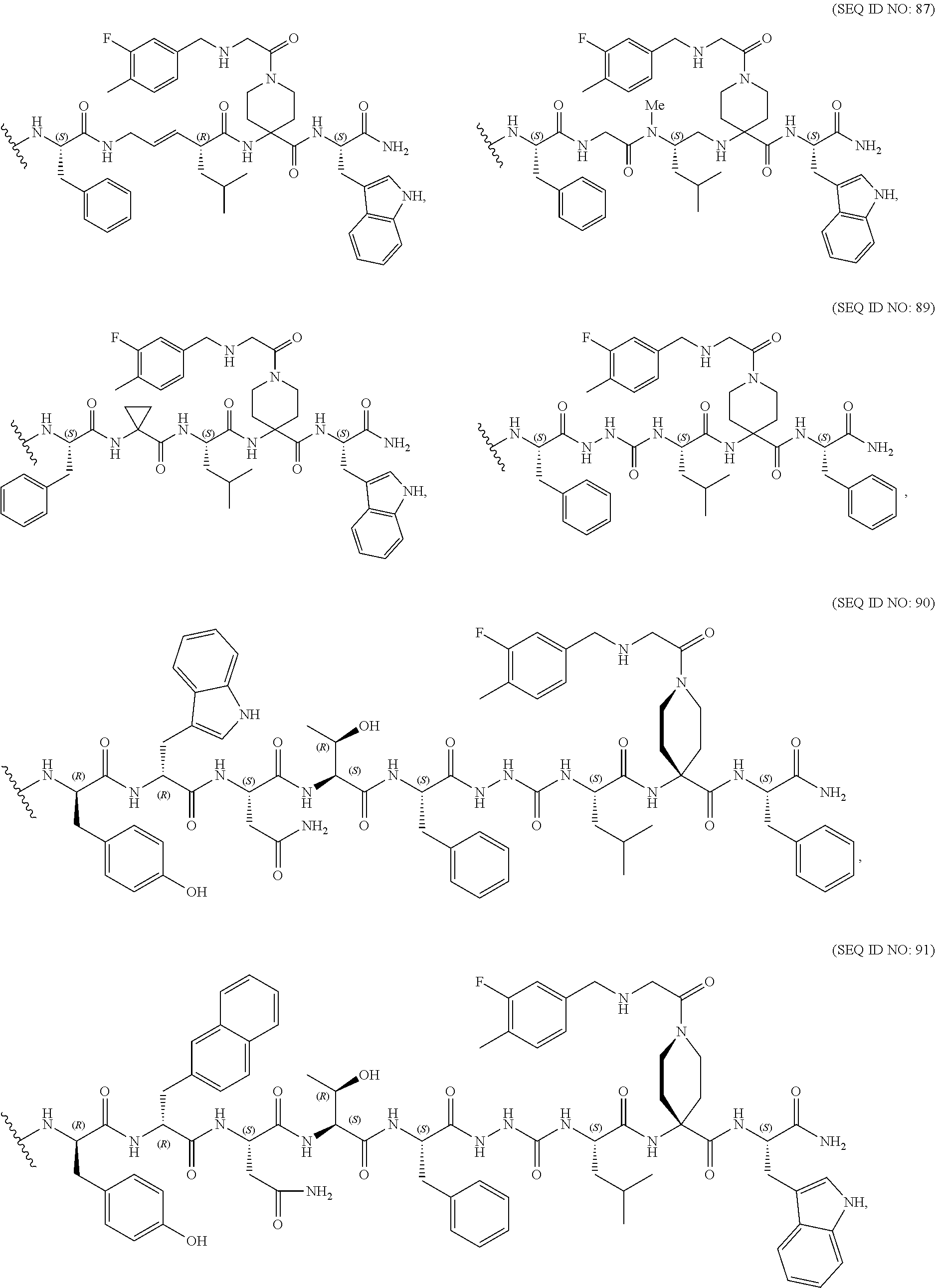

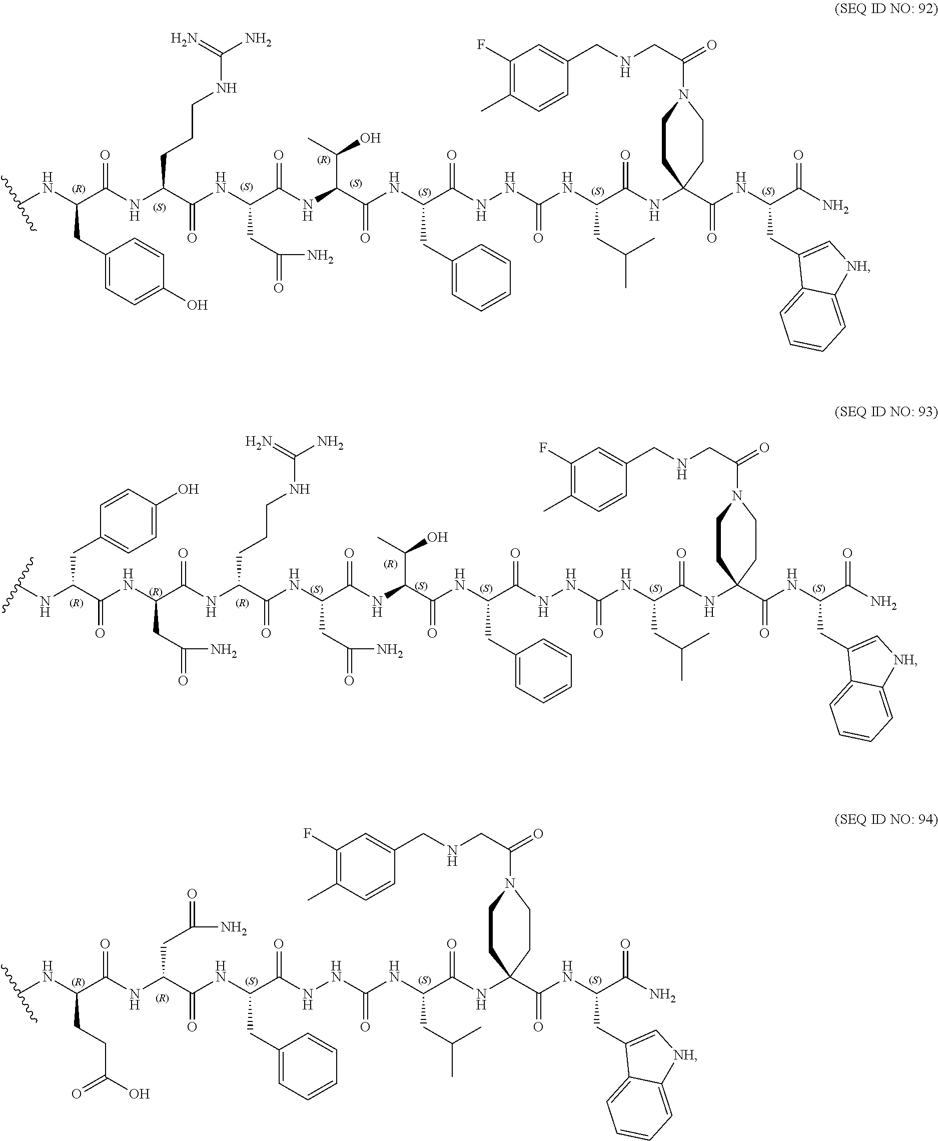

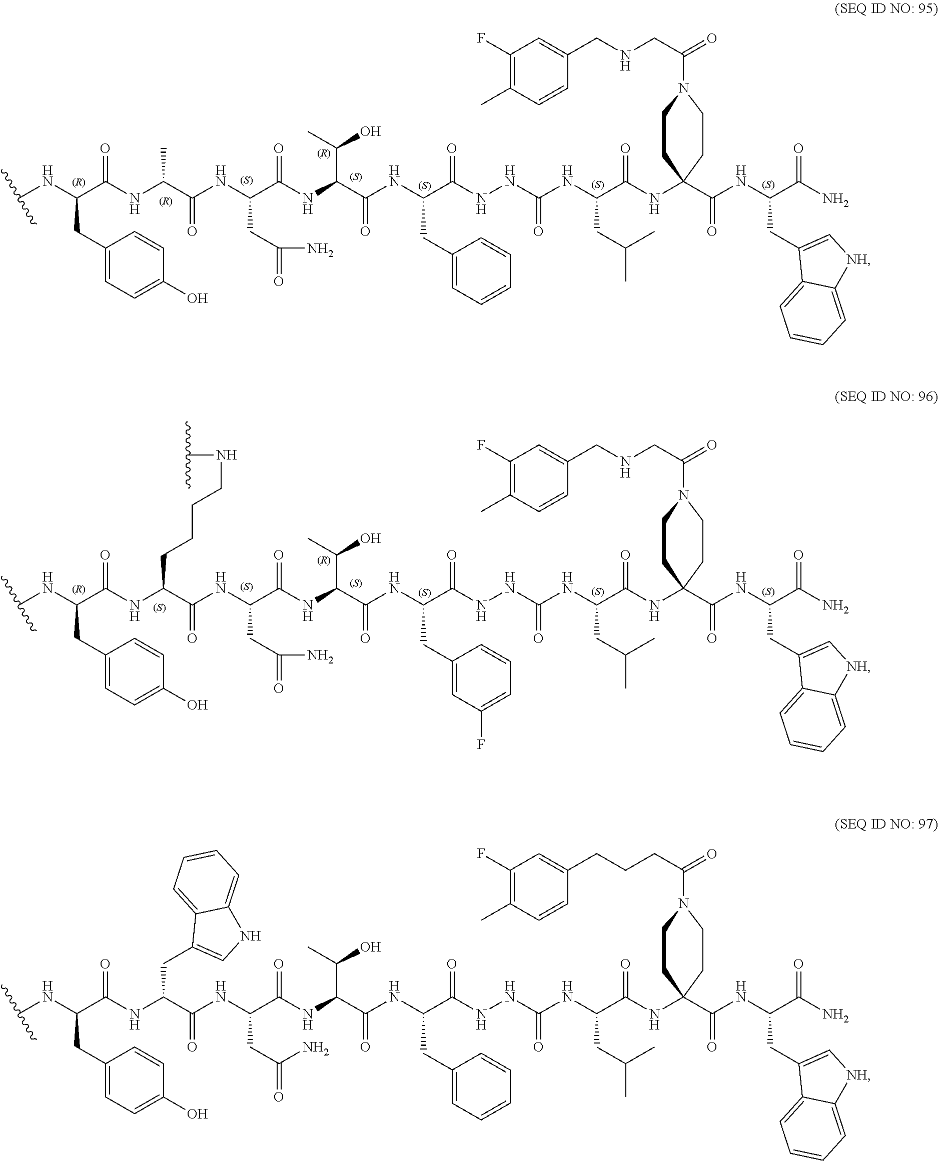

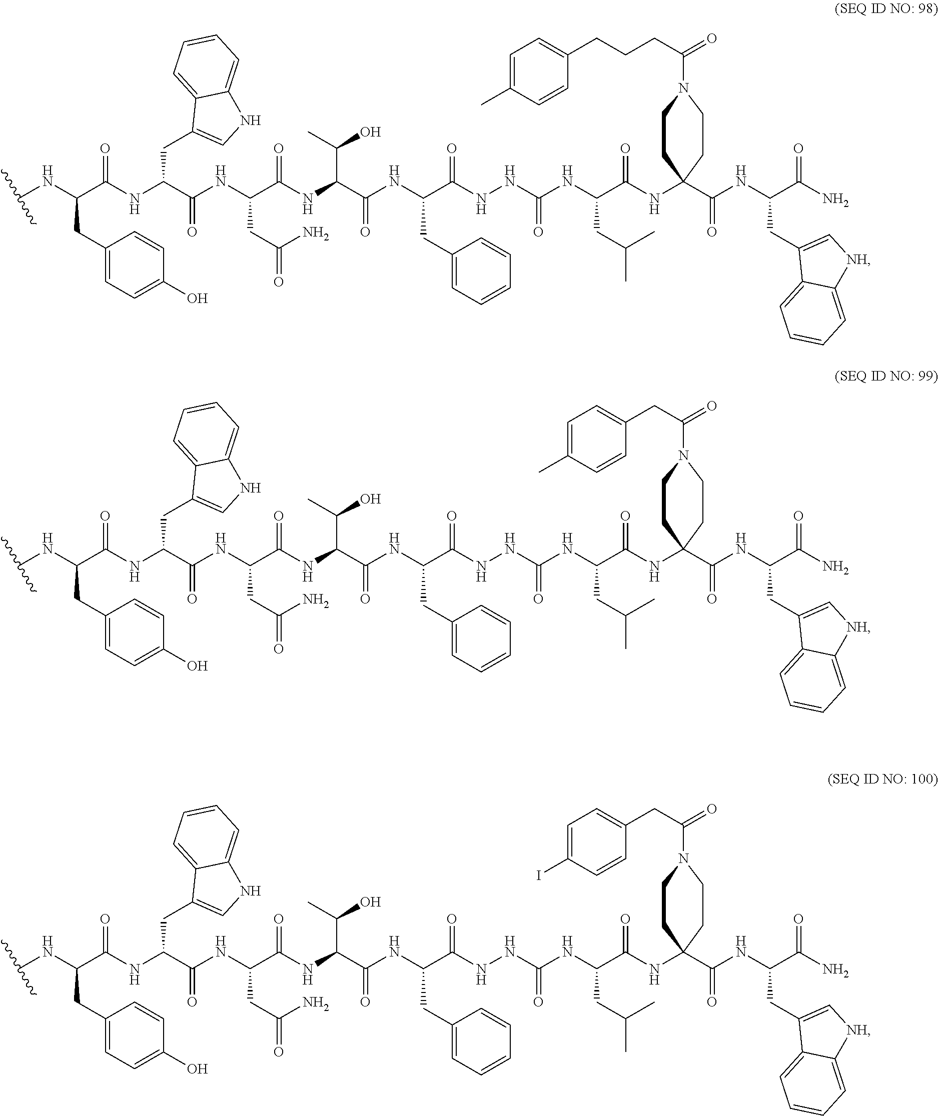

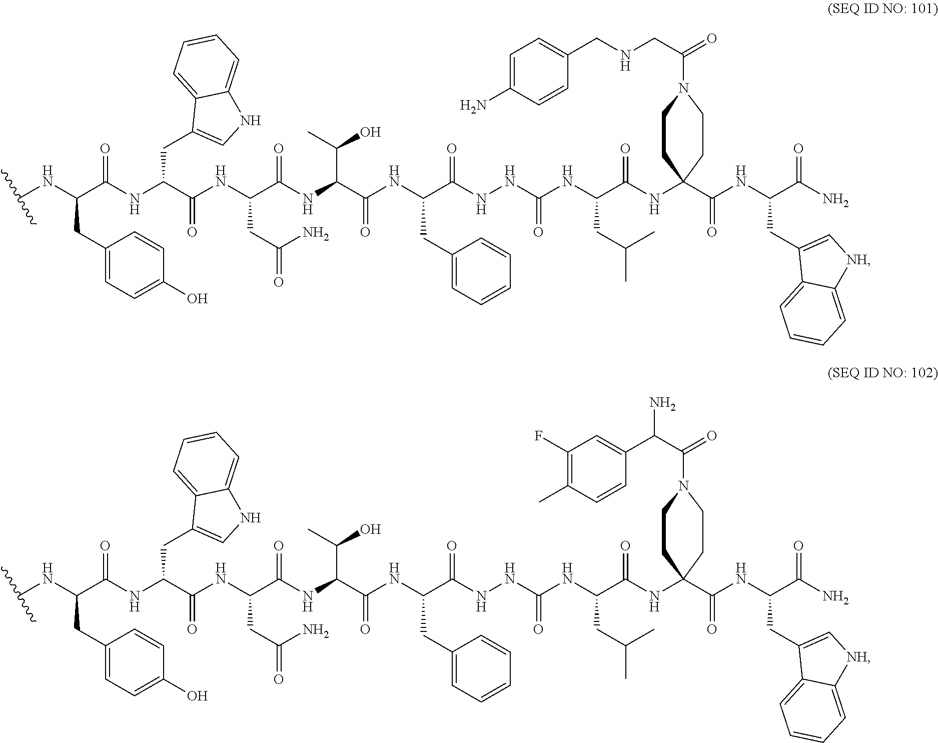

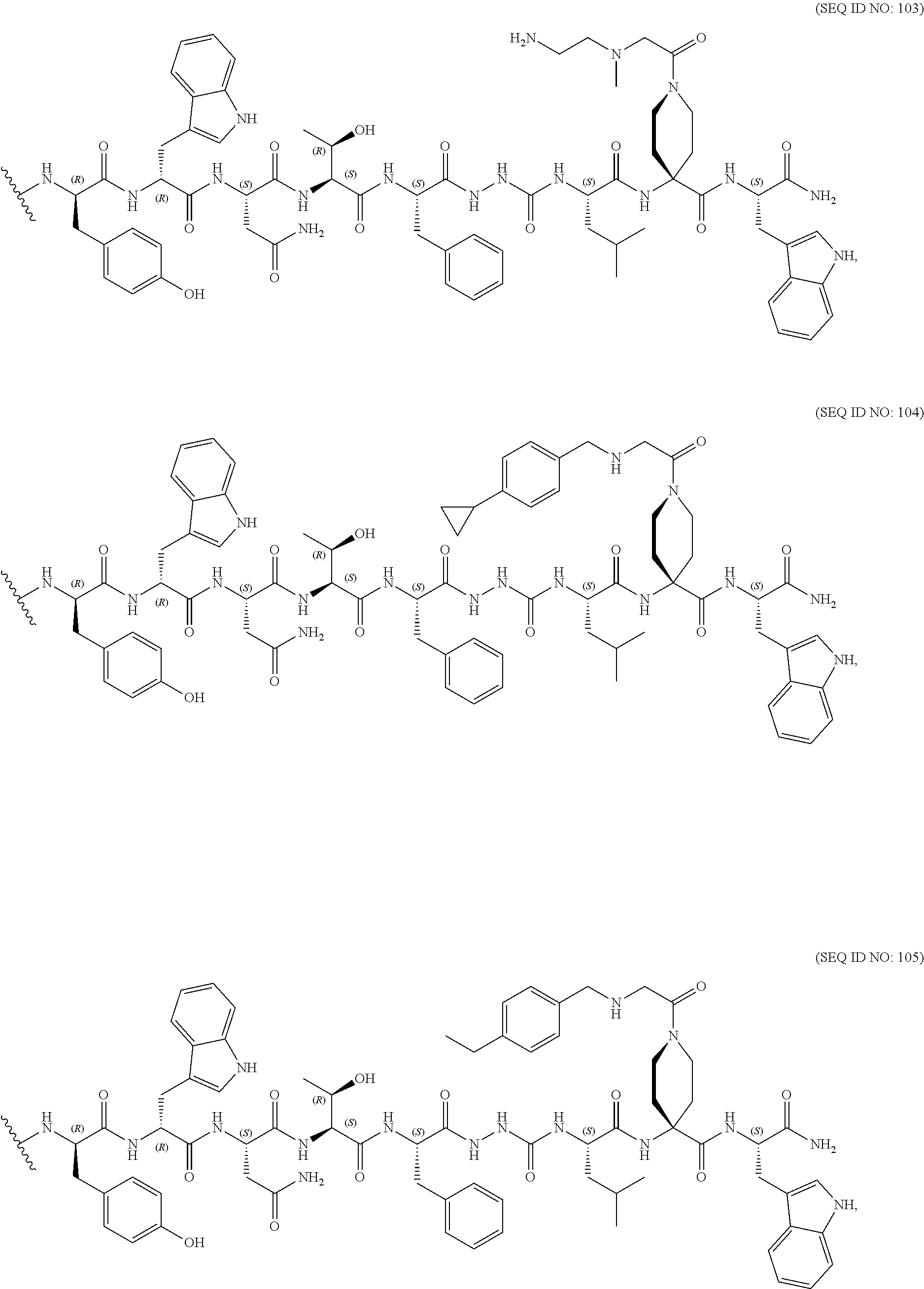

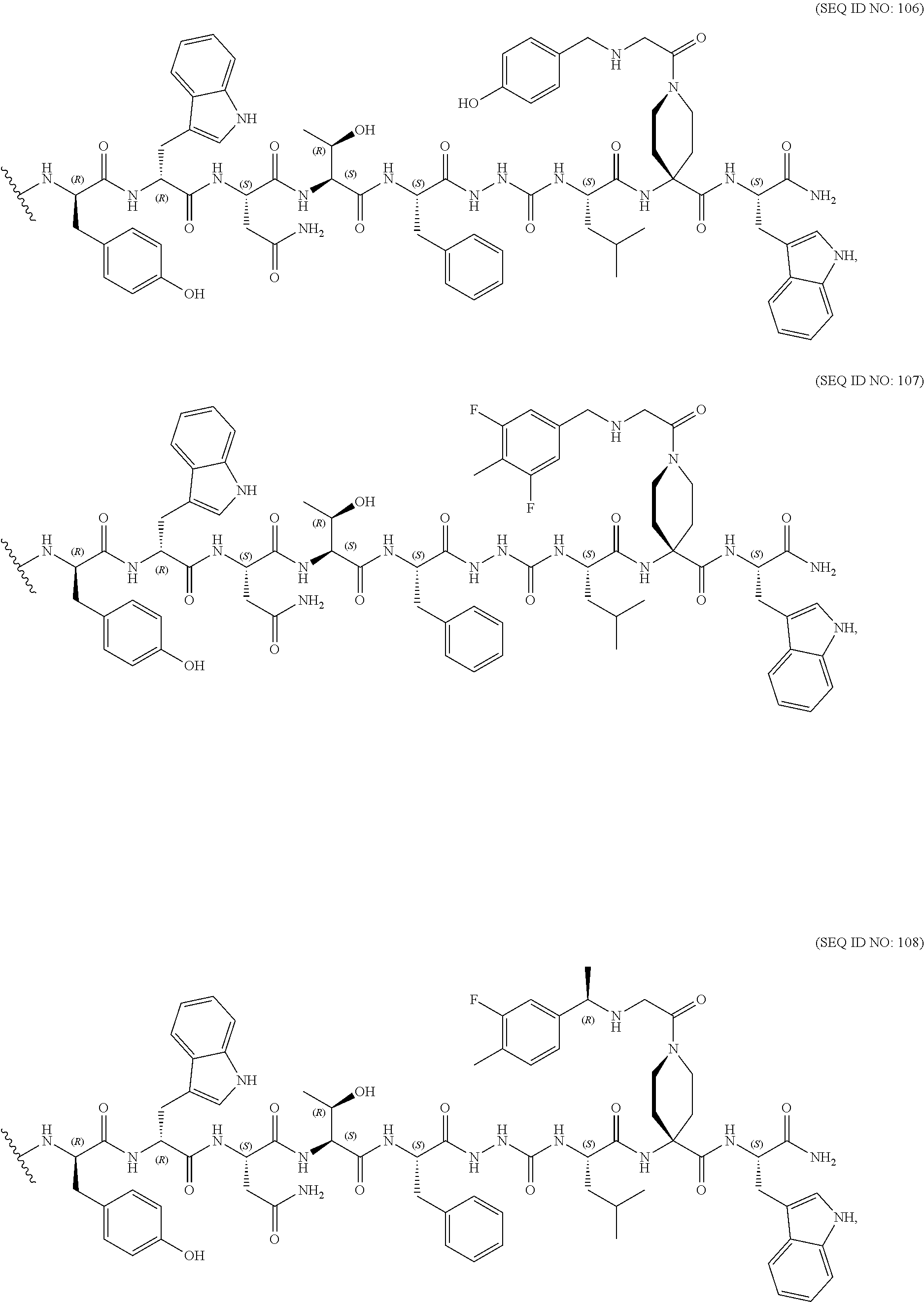

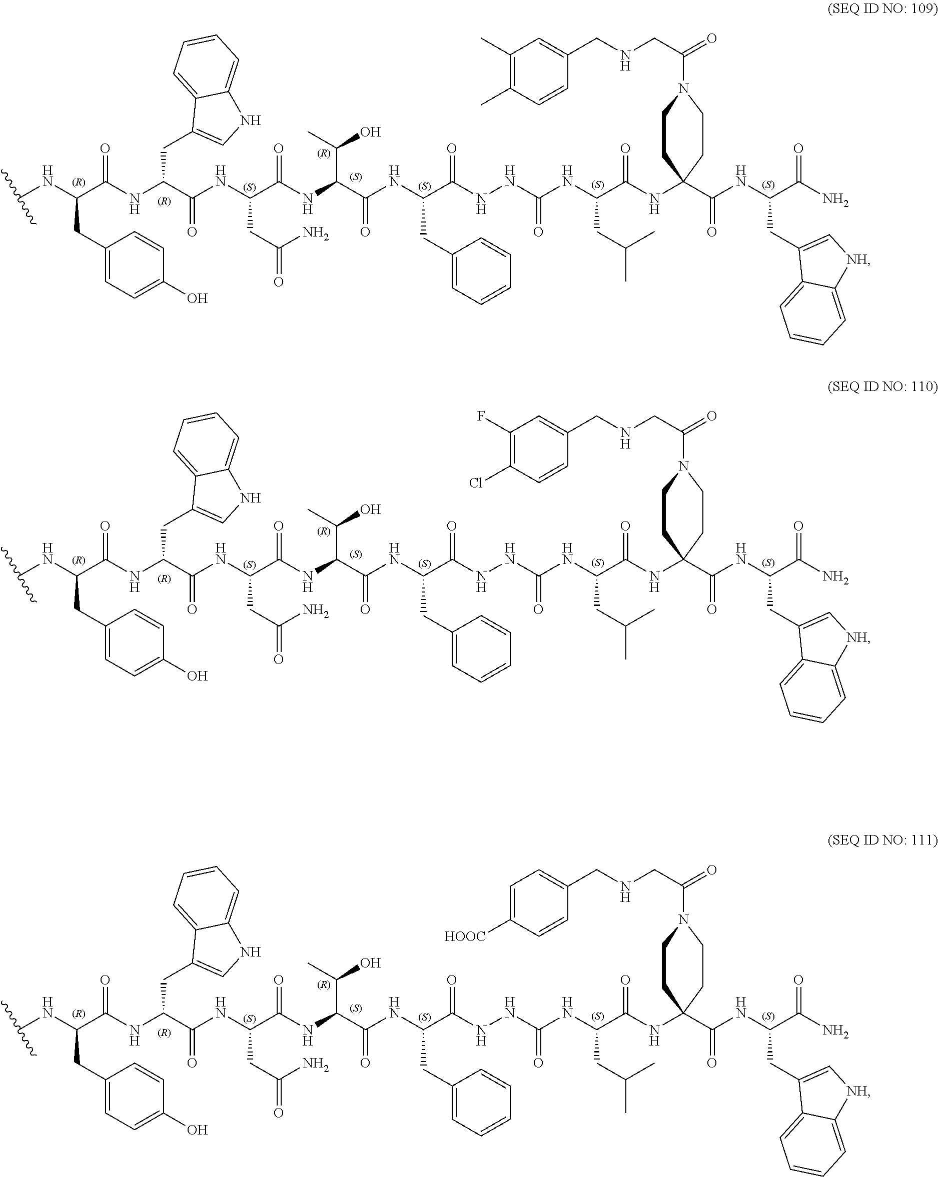

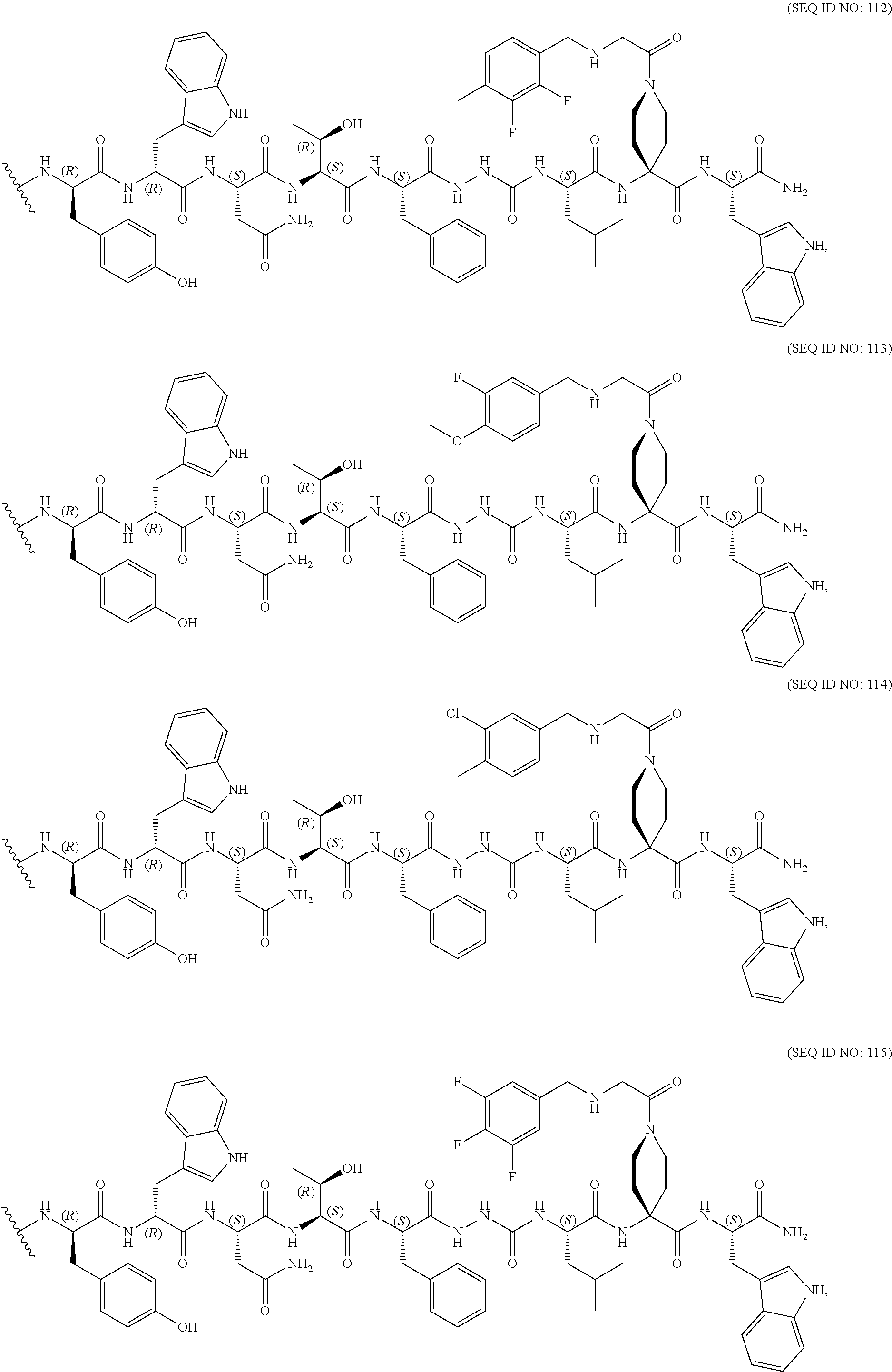

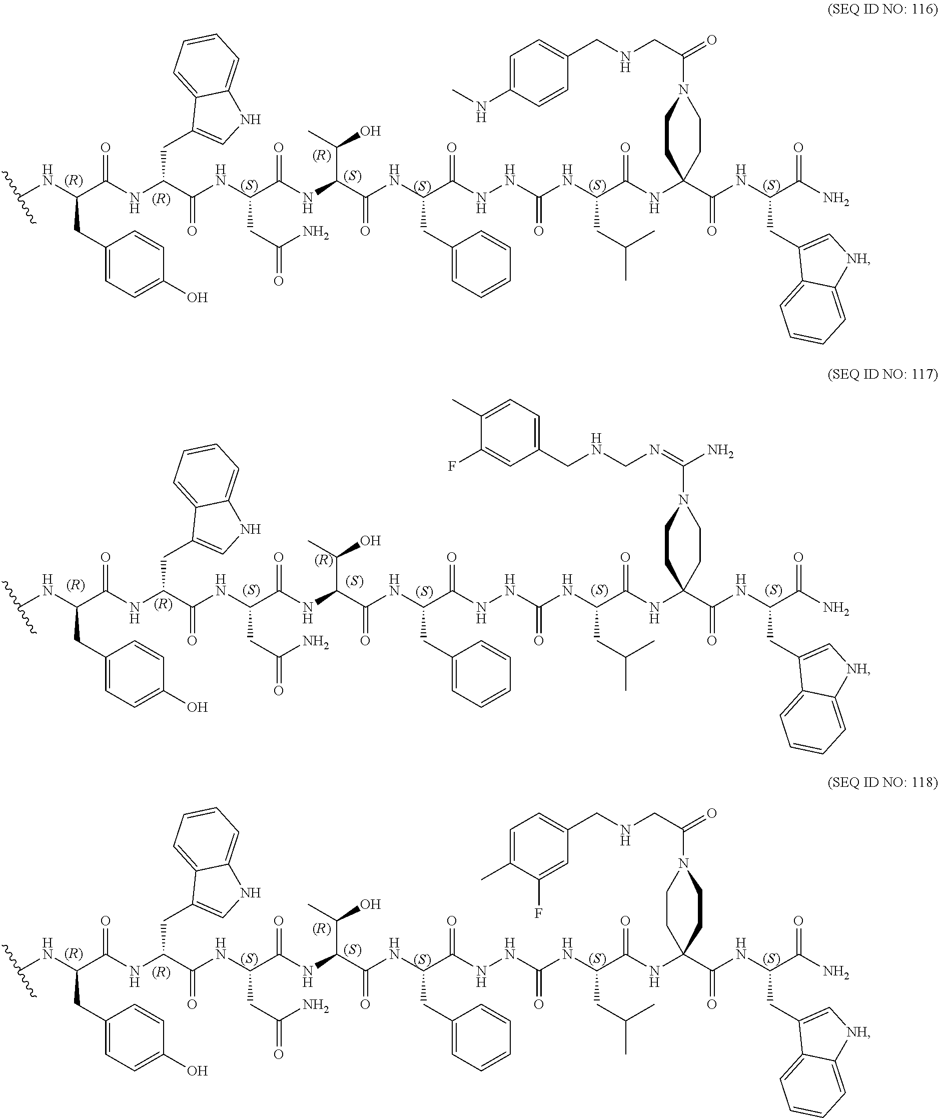

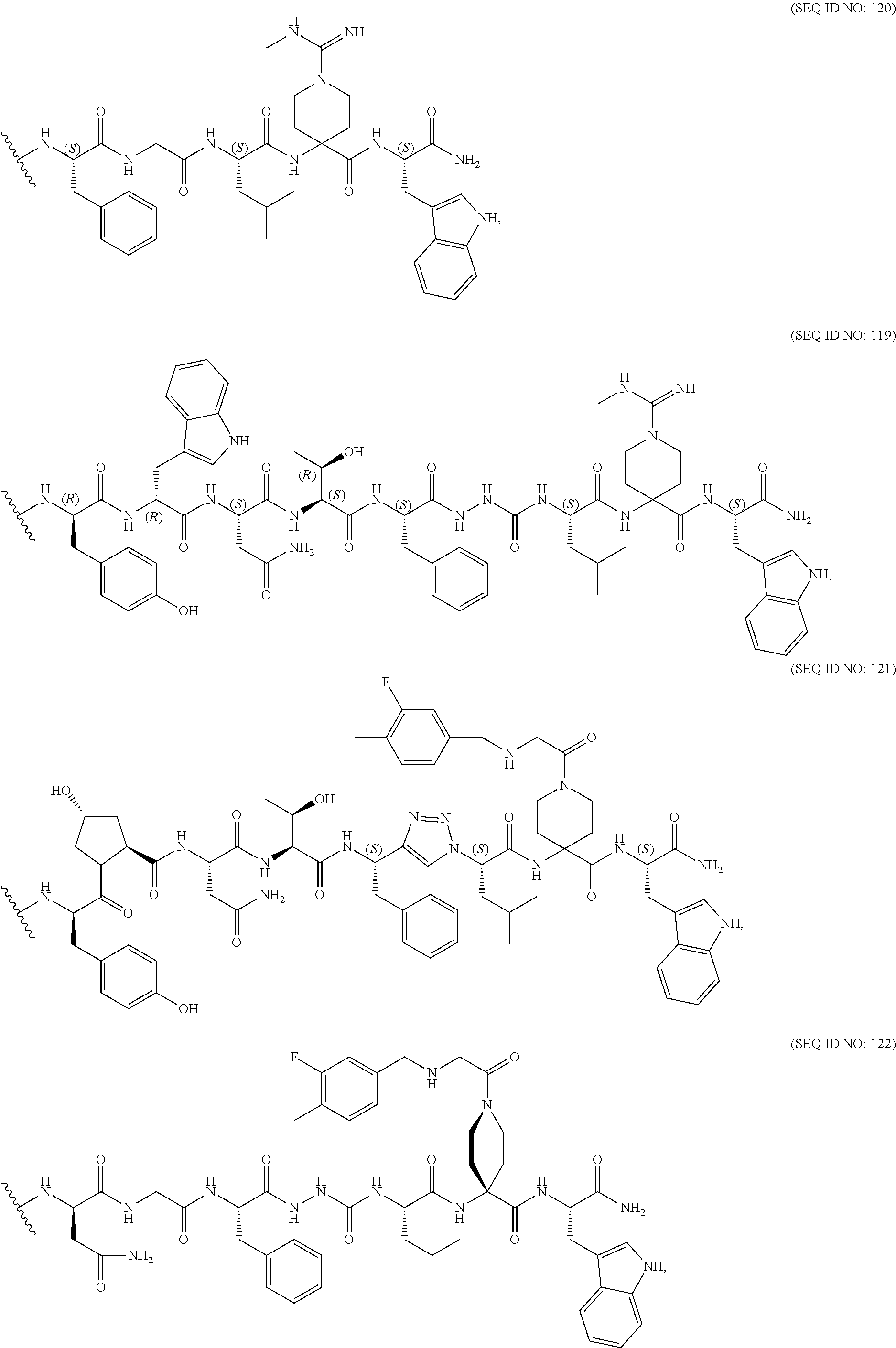

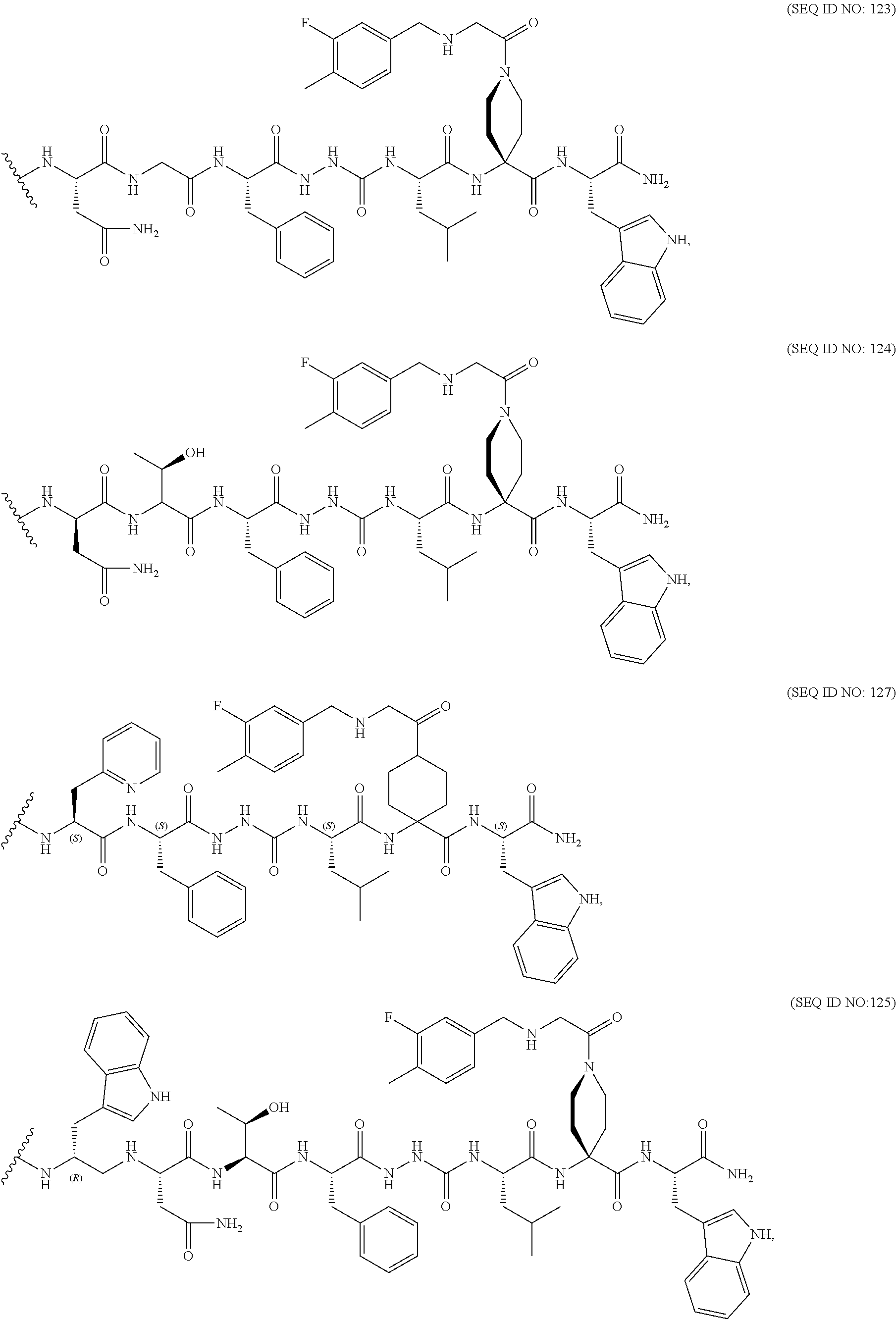

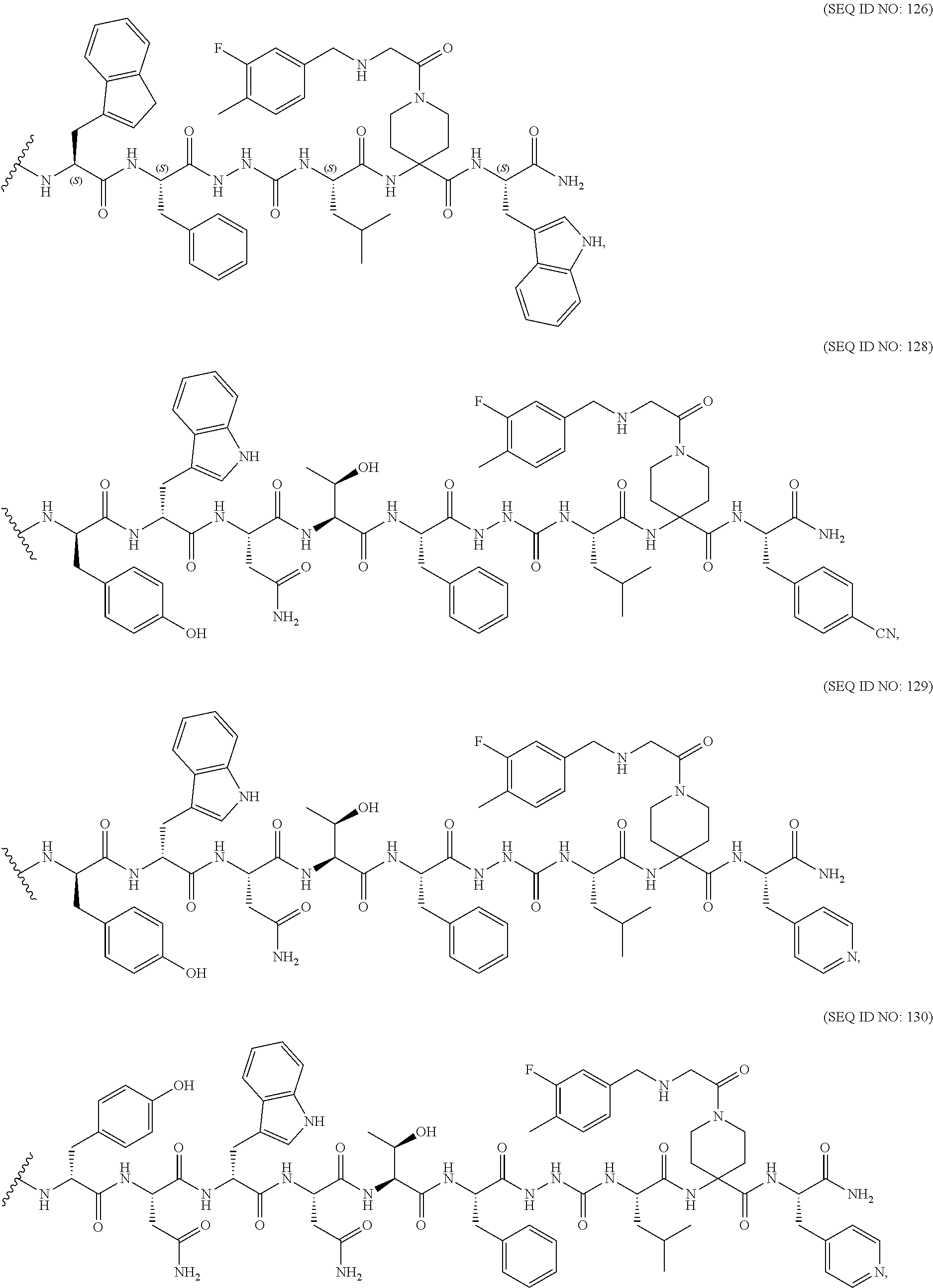

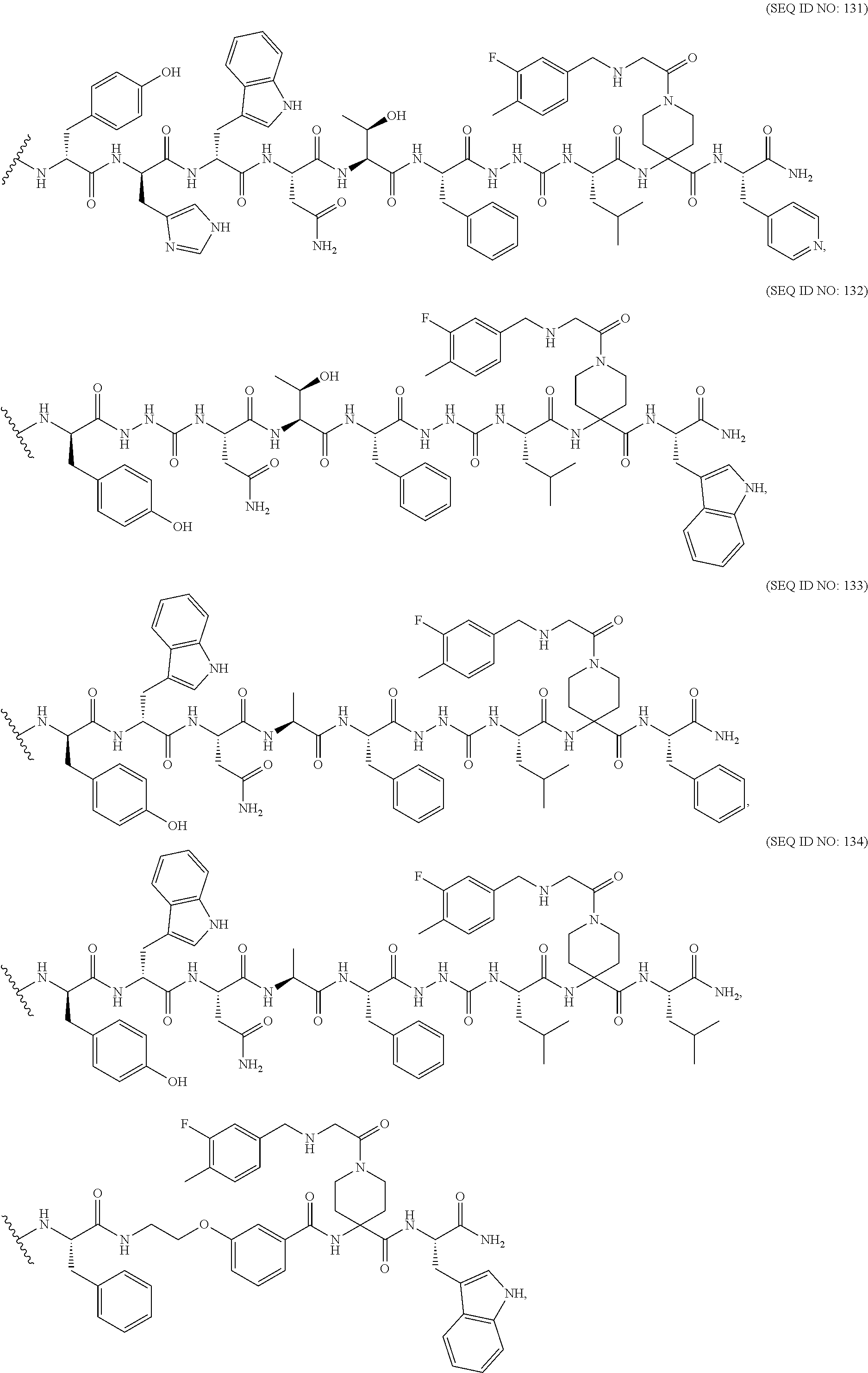

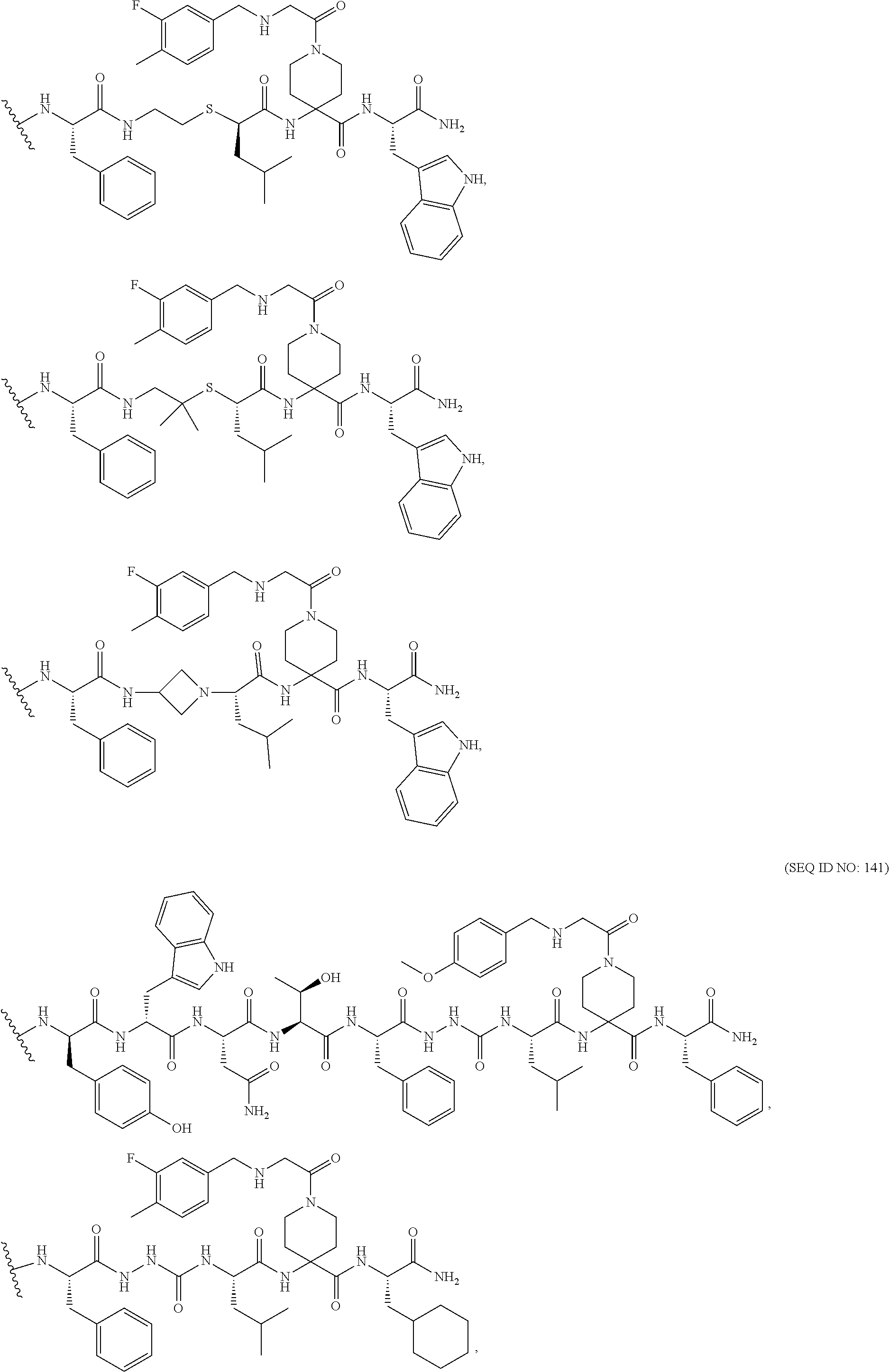

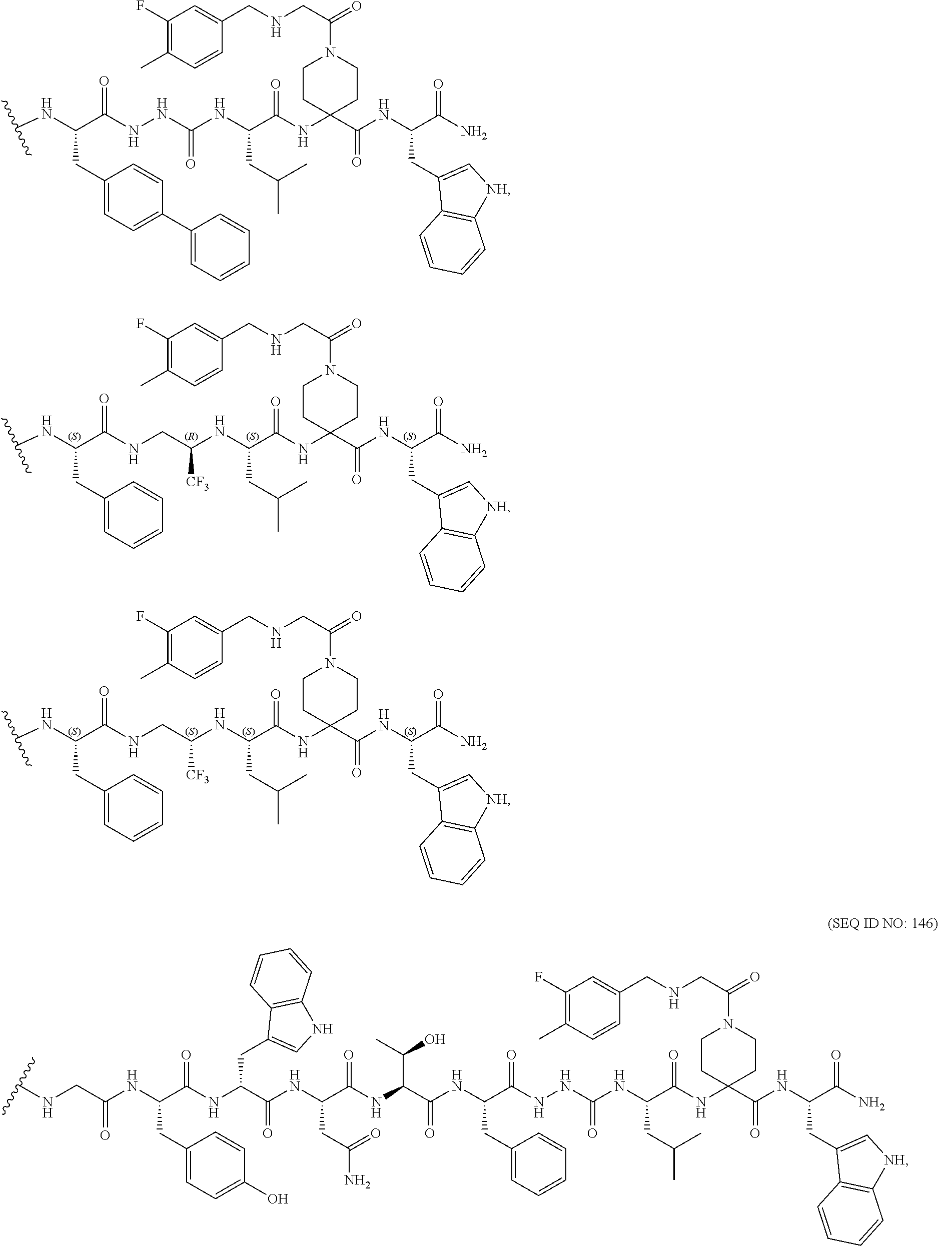

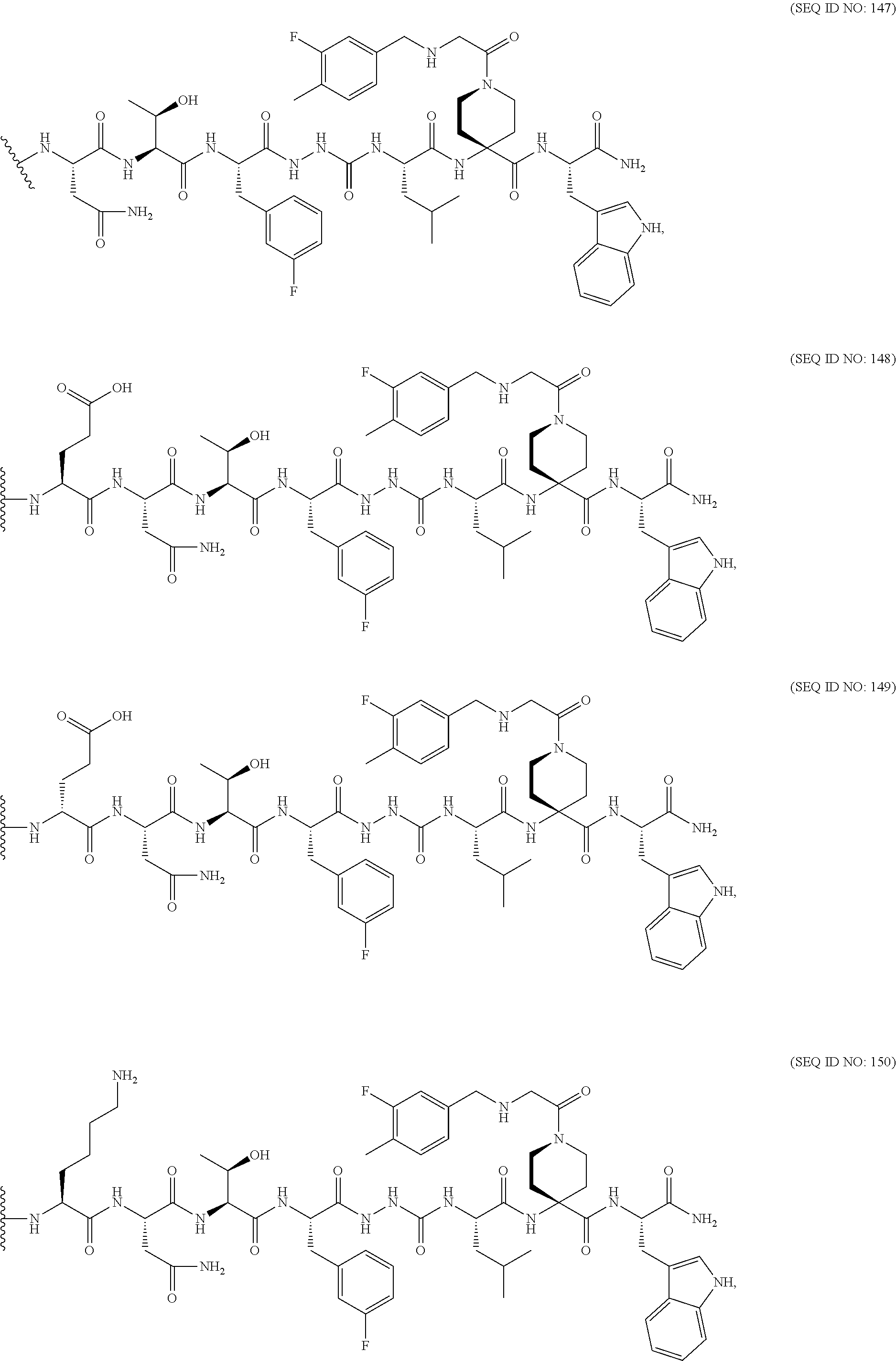

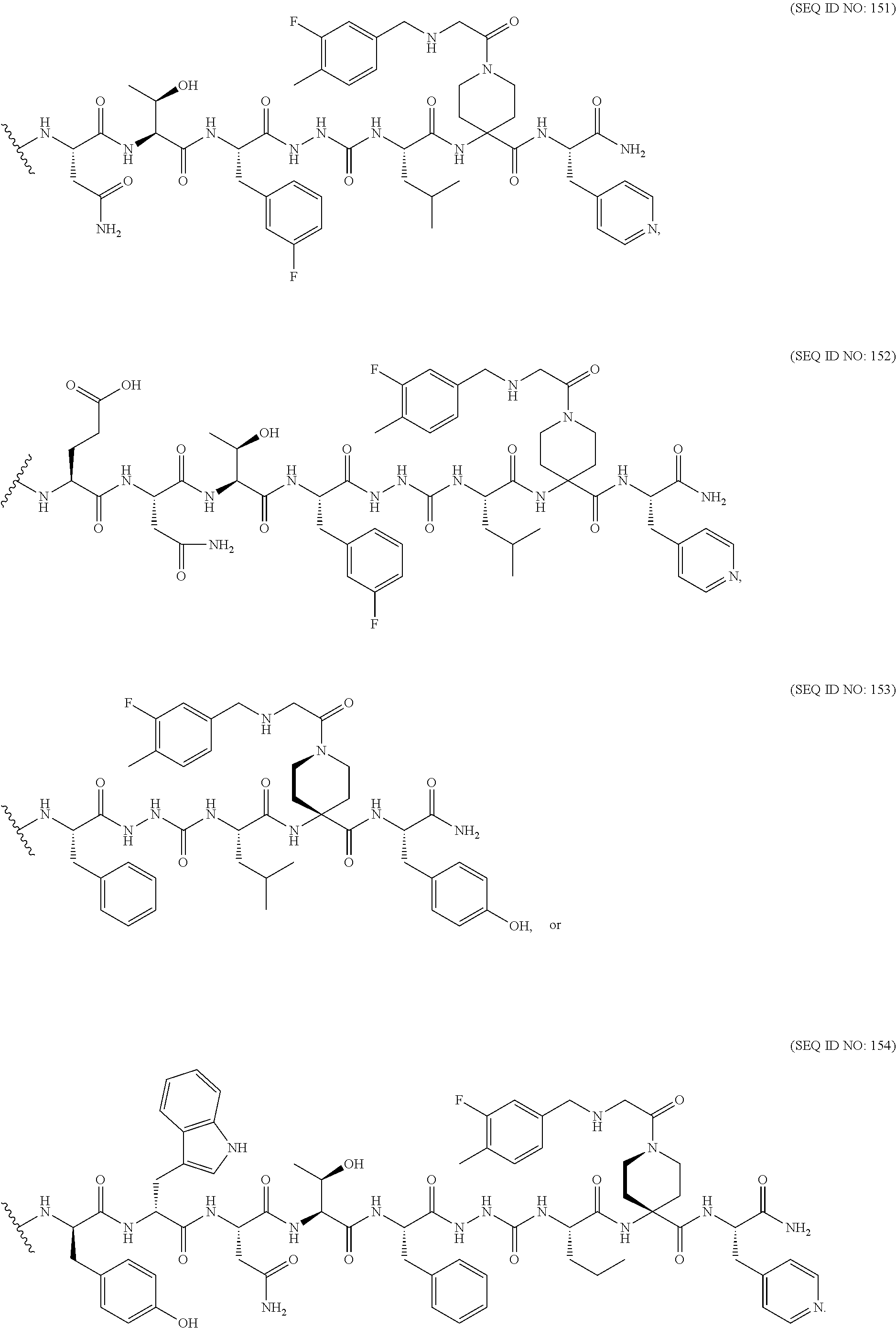

16. The compound of claim 1, or a pharmaceutically acceptable salt thereof, wherein R^{a} is a chelating moiety independently selected from the group consisting of:
1,4,7,10-tetraazacyclododecane-1,4,7,10-tetraacetic acid (DOTA);
2,2′,2″-(10-(2-amino-2-oxoethyl)-1,4,7,10-tetraazacyclododecane-1,4,7-triyl)triacetic acid (PSC);
1,4,7,10-tetraazacyclododecane-1,4,7-triacetic acid (DOTA);
1,4,7,10-tetraazacyclododecane-1,7-diacetic acid (DOTA);
α,α′,α″, α″′-tetramethyl-1,4,7,10-tetraazacyclododecane-1,4,7,10-tetraacetic acid (DOTMA);
1,4,7,10-tetrakis(carbamoylmethyl)-1,4,7,10-tetraazacyclododecane (DOTAM);
1,4,7,10-tetraazacyclododecane-1,4,7,10-tetrapropionic acid (DOTPA);
2,2′,2″-(10-(2-amino-2-oxoethyl)-1,4,7,10-tetraazacyclododecane-1,4,7-triylitriacetic acid;
benzyl-1,4,7,10-tetraazacyclododecane-1,4,7,10-tetraacetic acid (Bn-DOTA);
p-hydroxy-benzyl-1,4,7,10-tetraazacyclododecane-1,4,7%, 10-tetraacetic acid (p-OH-Bn-DOTA);
6,6′-((pyridine-2,6-diylbis(methylene))bis((carboxymethyl)azanediyl))bis(methylene))dipicolinic acid (H₄pypa);
H₄pypa-benzyl;
6,6′6,6″′-(((pyridine-2,6-diylbis(methylene))bis(azanetriyl))tetrakis(methylene))-tetrapicolinic acid (H₄py4pa);
H₄py4pa-benzyl;
2,2′,2″-1,4,7-triazacyclononane-1,4,7-triyl)triacetic acid (NOTA);
6,6′-((1,4,10,13-tetraoxa-7,16-diazacyclooctadecane-7,16-diyl)bis(methylene))dipicolinic acid (macropa);
2,2′,2″,2″′-(1,10-dioxa-4,7,13,16-tetraazacyclooctadecane-4,7,13,16-tetrayl)tetraacetic acid (crown);
6,6′-((ethane-1,2-diylbis((carboxymethyl)azanediyl))bis(methylene))dipicolinic acid (H₄octapa);
H₄octapa-benzyl; and
3,6,9,12-tetrakis(carboxymethyl)-3,6,9,12-tetraazatetradecanedioic acid (TTHA);
or a radionuclide complex thereof.

17. The compound of claim 1, or a pharmaceutically acceptable salt thereof, wherein R^{a} is 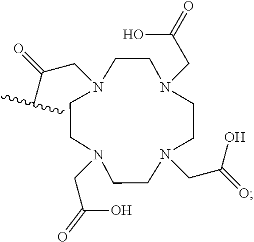 [SMILES: *C(=O)CN1CCN(CC(=O)O)CCN(CC(=O)O)CCN(CC(=O)O)CC1]
or a radionuclide complex thereof.

18. The compound of claim 1, or a pharmaceutically acceptable salt thereof, wherein:
-L- is absent, *-L¹-, —R¹⁷-L¹-, *—NR¹⁷-L⁵-L¹, *—NR¹⁷-L⁵-C(═O)-L¹, *—NR¹⁷-L⁵NR¹⁷—C(═O)-L¹-, *-L⁵-C(═O)-L¹-, *L⁵-L¹-, *—NR¹⁷-L⁵-NR¹⁷-L¹-, *—NR¹⁷-L⁵-C(═O)NR¹⁷-L⁵-C(═O)NR¹⁷-L¹-, *-(L³)-^{w}, *—NR¹⁷-L⁵-C(═O)-L³-NR¹⁷-L⁵-C(═O)—, or *-(L³)_{w}, —NR¹⁷-L⁵-C(═O)-L¹-;
wherein * denotes the attachment point to R^{a};
L⁵ is substituted or unsubstituted C₁-C₆ alkylene;
or L⁵ and R¹⁷ are taken together with the N atom to which they are attached to form N-heterocyclyl;
R¹⁷ is selected from hydrogen, C₁-C₆ alkyl, C₁-C₆ alkyl-CO₂H, —(CH₂CH₂O)—CH₂CH₂—CO₂H;
L¹ is absent or -L²-, -L²-(L³)_{w}-, -(L³)_{w}-L²-, or -L²-(L³)_{w}-L²-(L³)_{w}-;
each L² is independently absent, —C₀-C₆ alkylene-(substituted or unsubstituted aryl)-C₀-C₆ alkylene-C(═O)—, —C₀-C₆ alkylene-(substituted or unsubstituted arylene)-C₀-C₆alkylene-OC(═O)—, —C₀-C₀alkylene-(substituted or unsubstituted cyclohexylene)-C₀-C₆alkylene-C(═)—, —C₀-C₆ alkylene-(substituted or unsubstituted heterocycloalkylene)-C₀-C₆ alkylene-C(═O)—, —C₀-C₆ alkylene-(substituted or unsubstituted heteroarylene)-C₀-C₆ alkylene-C(═O)—, —C₀-C₆ alkylene-(substituted or unsubstituted heteroarylene)-C₀-C₆ alkylene-OC(═O)—, —C₄-C₂₀ polyethylene glycol, —C₄-C₂₀ polyethylene glycol-C(═O)—, substituted or unsubstituted —C₀-C₂₀ alkylene, substituted or unsubstituted —C₁-C₂₆ alkylene-C(═O)—, substituted or unsubstituted 2 to 20 membered heteroalkylene, —(CH₂CH₂O)_{z}—CH₂—, —(CH₂CH₂O), —CH₂CH₂—, —(CH₂CH₂O)_{z}—CH₂—C(═O)—, or —(CH₂CH₂O)_{z}—CH₂CH₂—C(═O)—;
each z, is independently 1, 2, 3, 4, 5, or 6;
each L³ is independently selected from natural or unnatural amino acids, wherein any free amine of an amino acid or peptide bond is optionally independently substituted with L⁴, and wherein when two or more amino acids are present then the N atom of the amide linking the amino acids is optionally substituted with —CH₃;
each L⁴ is independently selected from —C(═O)—C₁-C₆ alkylene-C(═O)—, —C(═O)˜NH—C₁-C₆ alkylene-C(═O)—, and —C(═O)—C₁-C₆ alkylene-(substituted or unsubstituted triazolylene)-C₁-C₆alkylene-C(═O)—; wherein if L⁴ is present then: L⁴ is attached to the any one of X¹, X², X³, X⁴, X⁵, X⁶, or X⁷, or L⁴ is attached to X⁸ if X¹, X², X³, X⁴, X⁵, X⁶, and X⁷ are absent; and
each w is independently 1, 2, 3, 4, 5, or 6.

19. The compound of claim 18, or a pharmaceutically acceptable salt thereof, wherein:
-L- is absent, *—NR¹⁷—, —NR¹⁷-L⁵, *—NR⁵-L⁵-C(═O)—, *—NR¹⁷—C₀-C₆alkylene-(substituted or unsubstituted phenylene)-C₀-C₆alkylene-C(═O)—, *—NR¹⁷—C₀-C₆alkylene-(substituted or unsubstituted cyclohexylene)-C₀-C₆ alkylene-C(═O)—, *—NR¹⁷—C₀-C₆ alkylene-(substituted or unsubstituted heterocycloalkylene)-C₀-C₆ alkylene-C(═O)—, *—NR¹⁷—C₀-C₆ alkylene-(substituted or unsubstituted heteroarylene)-C₀-C₆ alkylene-C(═O)—, *—NR¹⁷-substituted or unsubstituted C₁-C₂₀ alkylene-C(═O)—, *—NR¹⁷—(CH₂CH₂O)—CH₂—C(═O)—, *—NR¹⁷-(CH₂CH₂O)_{z}—CH₂CH₂—C(═O)—, —R¹⁷-L⁵-C(═O)-(L³)_{w}-, *—NR⁵-L⁵-C(═O)NR¹⁷—C₀-C₆ alkylene-(substituted or unsubstituted phenylene)-C₀-C₆ alkylene-C(═O)—, *—NR⁵-L⁵-C(═O)NR¹⁷—C₀-C₆ alkylene-(substituted or unsubstituted cyclohexylene)-C₀-C₆ alkylene-C(═O)—, *—NR⁵-L⁵-C(═O)NR¹⁷—C₀-C₆ alkylene-(substituted or unsubstituted heterocycloalkylene)-C₀-C₆alkylene-C(═O)—, or *—NR⁵-L⁵-C(═O)NR¹⁷—C₀-C₆ alkylene-(substituted or unsubstituted heteroarylene)-C₀-C₆ alkylene-C(═O)—; and
z is 1, 2, 3, 4, 5, or 6;
wherein * denotes the attachment point to R^{a}.

20. The compound of claim 18, or a pharmaceutically acceptable salt thereof, wherein:
-(L³)_{w}- is sarcosine, sarcosine-sarcosine, sarcosine-sarcosine-sarcosine, sarcosine-sarcosine-sarcosine-sarcosine (SEQ ID NO: 24), sarcosine-sarcosine-sarcosine-sarcosine-sarcosine (SEQ ID NO: 25), sarcosine-sarcosine-sarcosine-sarcosine-sarcosine-sarcosine (SEQ ID NO: 831), valine-citrulline, valine-alanine, methionine-valine-lysine, glycine-phenylalanine-glycine-glycine (SEQ ID NO: 832), tyrosine-arginine-valine, arginine-valine, 3-sulfo-alanine, 3-sulfo-alanine-3-sulfo-alanine, 3-sulfo-alanine-3-sulfo-alanine-3-sulfo-alanine, glycine-glutamate, glycine-glutamate-glycine, glycine-glutamate-glutamate, methionine-tryptophan-lysine, methionine-phenylalanine-lysine, methionine-valine, methionine-valine-lysine, or phenylalanine-lysine, wherein the free amine of lysine is optionally substituted with L⁴;
L⁴ is —C(═O)—(CH₂)₃—C(═O)—, —C(═O)—(CH₂)₄—C(═O)—, —C(═O)—(CH₂)₅—C(═O)—, —C(═O)—(CH₂)₆—C(═O)—, —C(═O)NH—(CH₂)₃—C(═O)—, —C(═O)NH—(CH₂)₄—C(═O)—, —C(═O)NH—(CH₂)₅—C(═O)—, —C(═C)NH—(CH₂)₆—C(═O)—, —C(═O)—(CH₂)₂-(triazolylene)-(CH₂)₁—C(═O)— or —C(═O)—(CH₂)₂-(triazolylene)-(CH₂)₂—C(═O)—; wherein if L⁴ is present then: L⁴ is attached to the any one of X¹, X², X³, X⁴, X⁵, X⁶, or X⁷, or L⁴ is attached to X⁸ if X¹, X², X³, X⁴, X⁵, X⁶, and X⁷ are absent.

21. The compound of claim 1, or a pharmaceutically acceptable salt thereof, wherein: -L- is: absent, 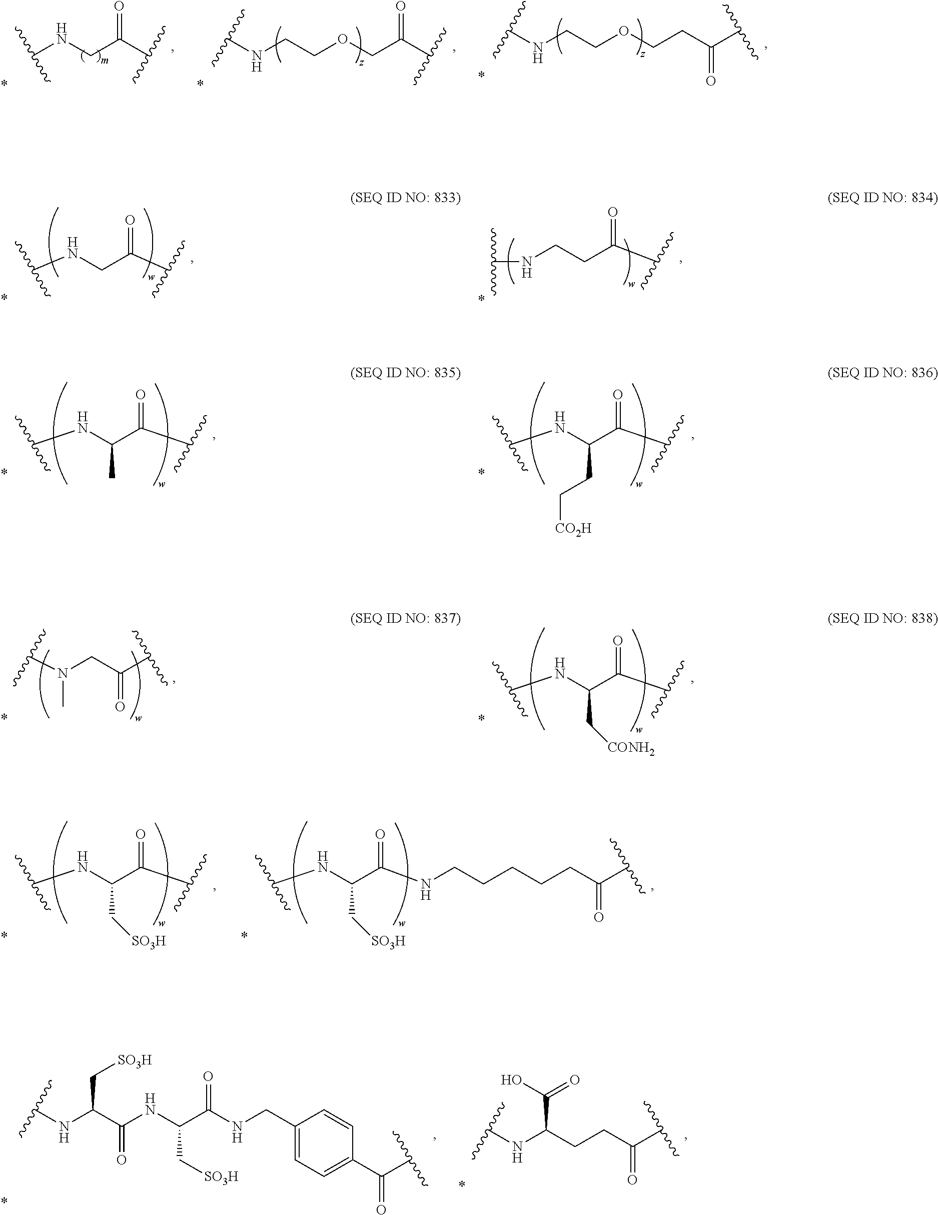 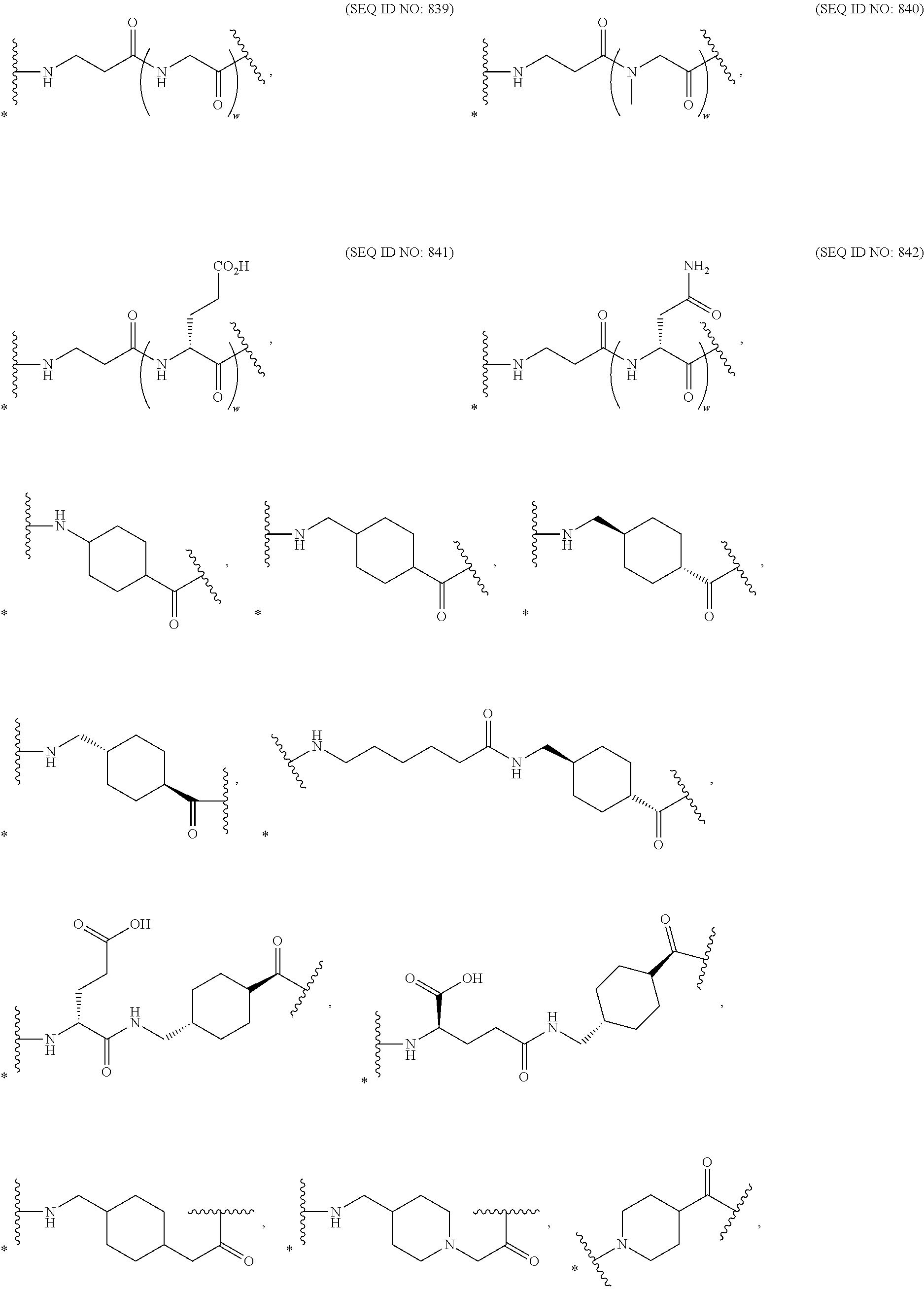 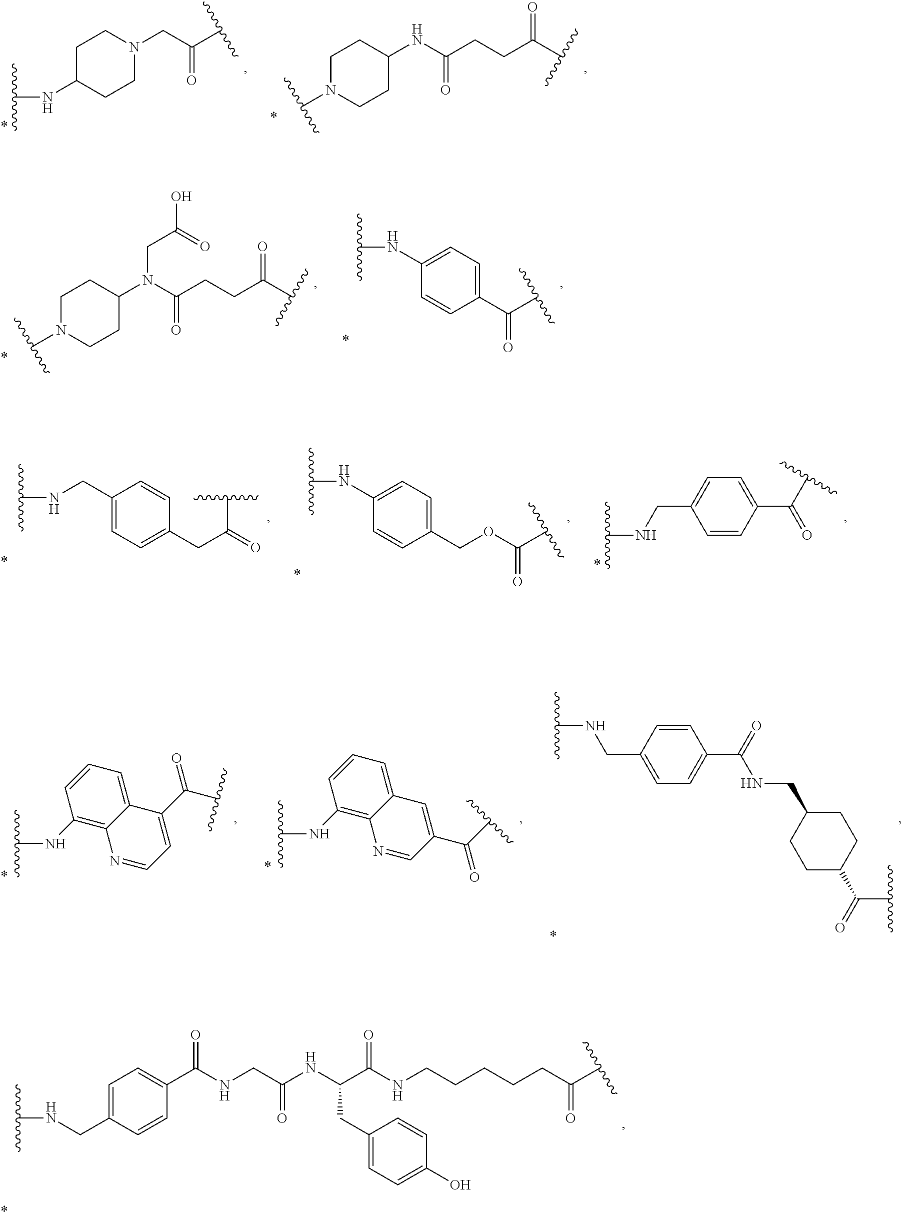 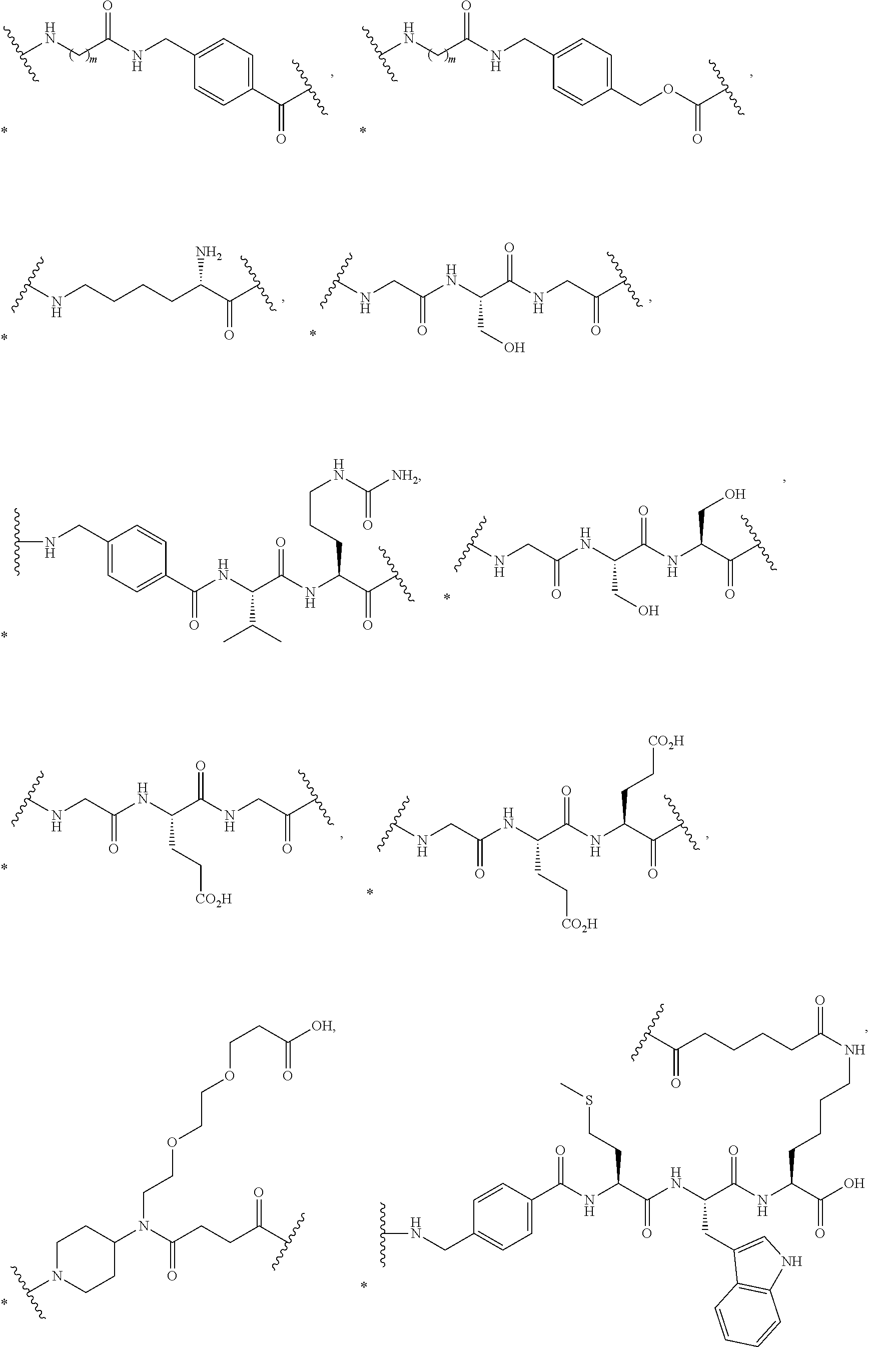 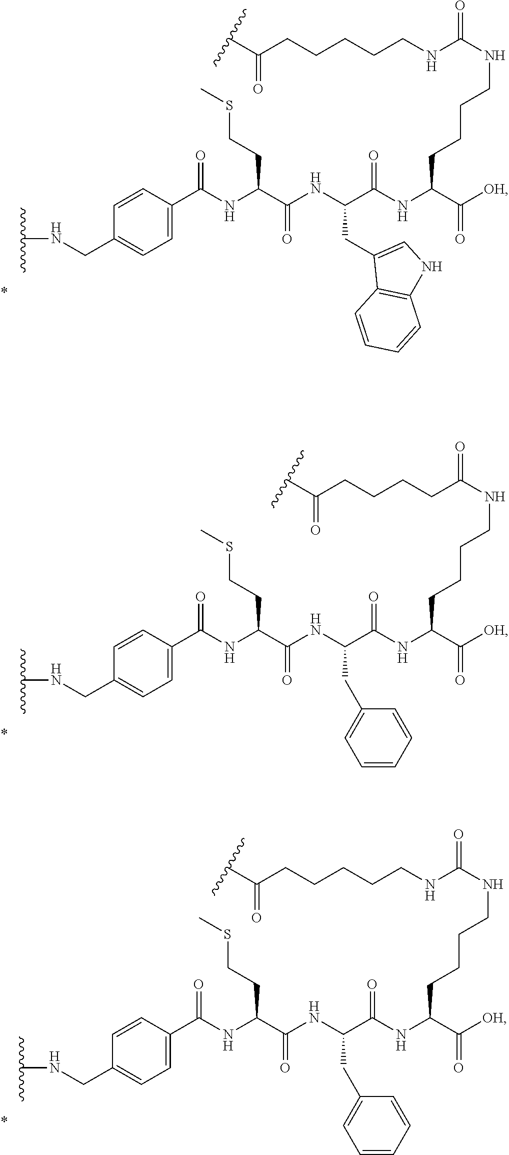 [SMILES: *NCc1ccc(C(=O)N[C@@H](CCSC)C(=O)N[C@@H](Cc2c[nH]c3ccccc23)C(=O)N[C@@H](CCCCNC(=O)NCCCCCC(*)=O)C(=O)O)cc1 ; *NCc1ccc(C(=O)N[C@@H](CCSC)C(=O)N[C@@H](Cc2ccccc2)C(=O)N[C@@H](CCCCNC(=O)CCCCC(*)=O)C(=O)O)cc1 ; *NCc1ccc(C(=O)N[C@@H](CCSC)C(=O)N[C@@H](Cc2ccccc2)C(=O)N[C@@H](CCCCNC(=O)NCCCCCC(*)=O)C(=O)O)cc1] 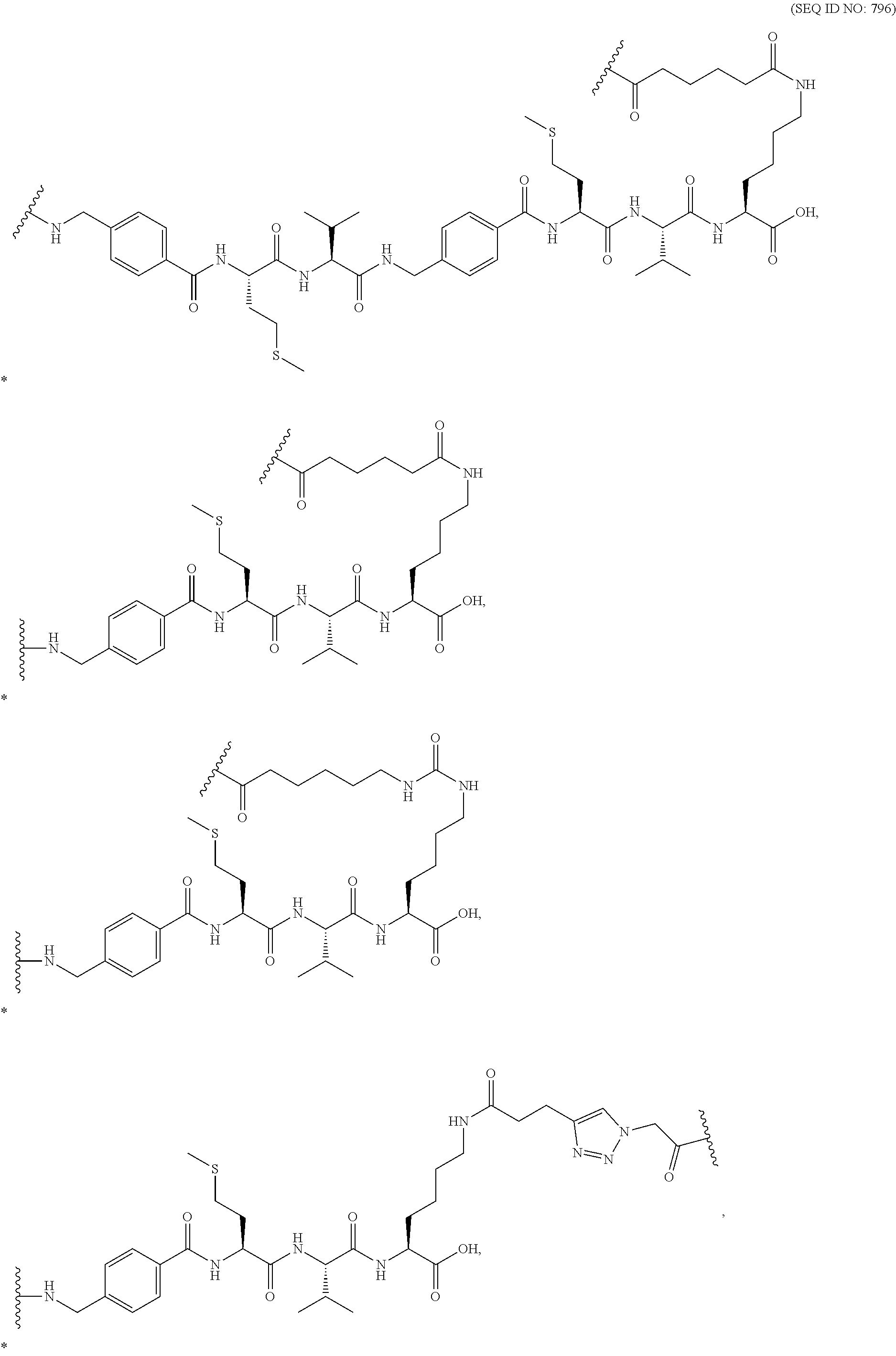 [SMILES: *NCc1ccc(C(=O)N[C@@H](CCSC)C(=O)N[C@H](C(=O)NCc2ccc(C(=O)N[C@@H](CCSC)C(=O)N[C@H](C(=O)N[C@@H](CCCCNC(=O)CCCCC(*)=O)C(=O)O)C(C)C)cc2)C(C)C)cc1 ; *NCc1ccc(C(=O)N[C@@H](CCSC)C(=O)N[C@H](C(=O)N[C@@H](CCCCNC(=O)CCCCC(*)=O)C(=O)O)C(C)C)cc1 ; *NCc1ccc(C(=O)N[C@@H](CCSC)C(=O)N[C@H](C(=O)N[C@@H](CCCCNC(=O)NCCCCCC(*)=O)C(=O)O)C(C)C)cc1 ; *NCc1ccc(C(=O)N[C@@H](CCSC)C(=O)N[C@H](C(=O)N[C@@H](CCCCNC(=O)CCc2cn(CC(*)=O)nn2)C(=O)O)C(C)C)cc1] 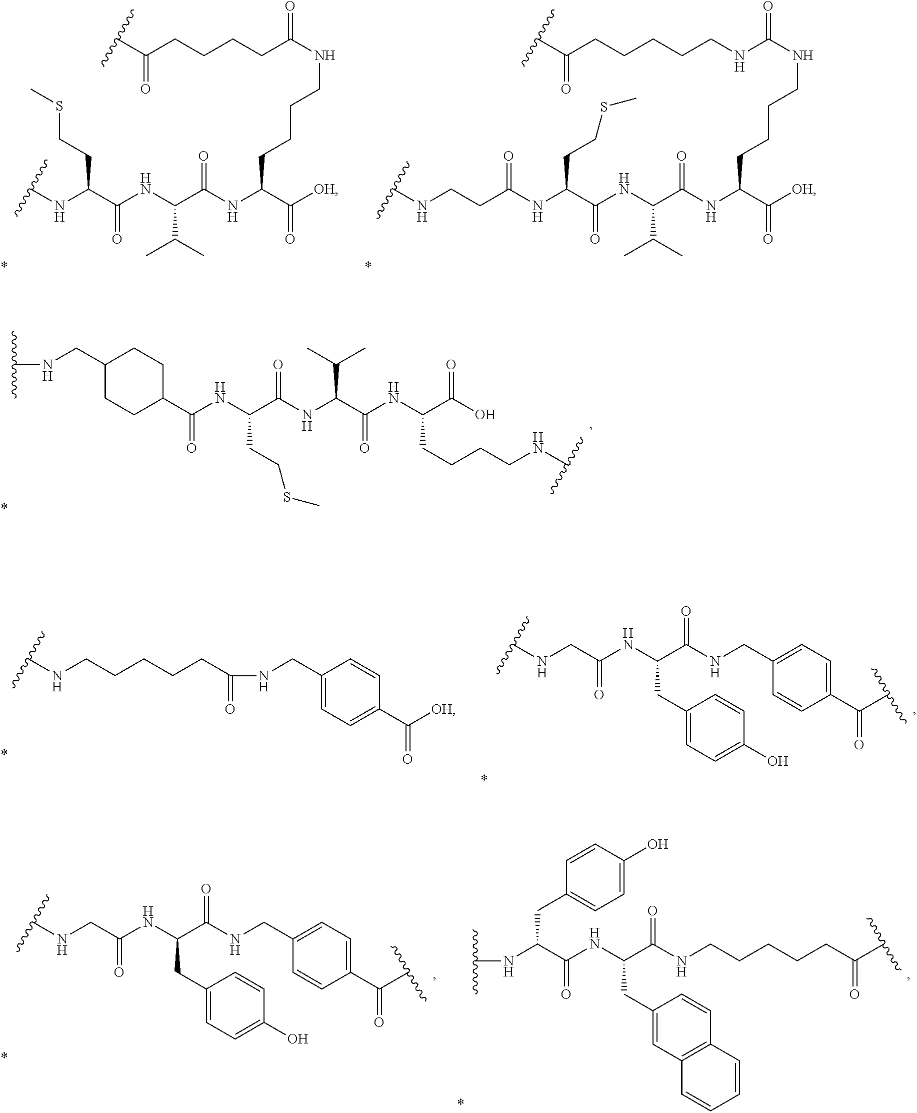 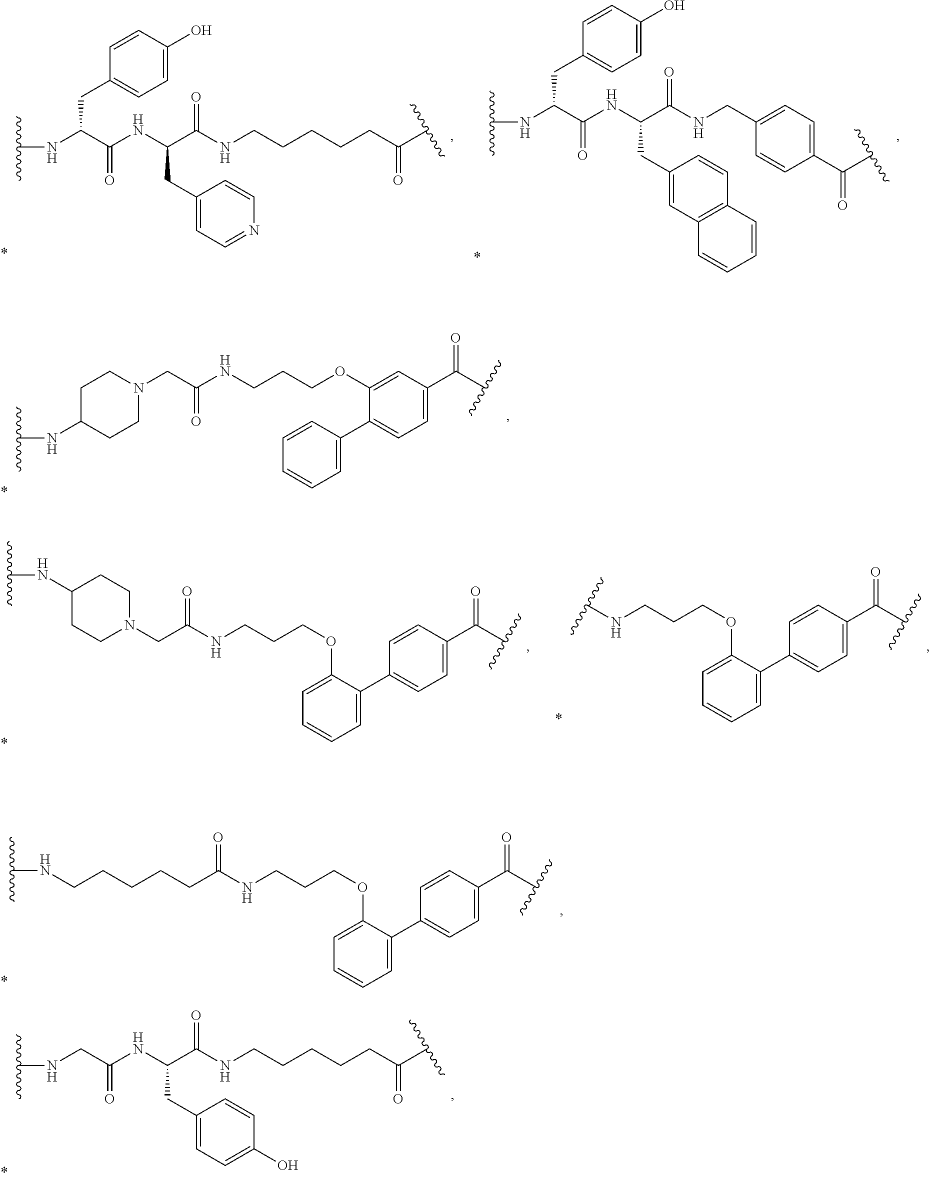 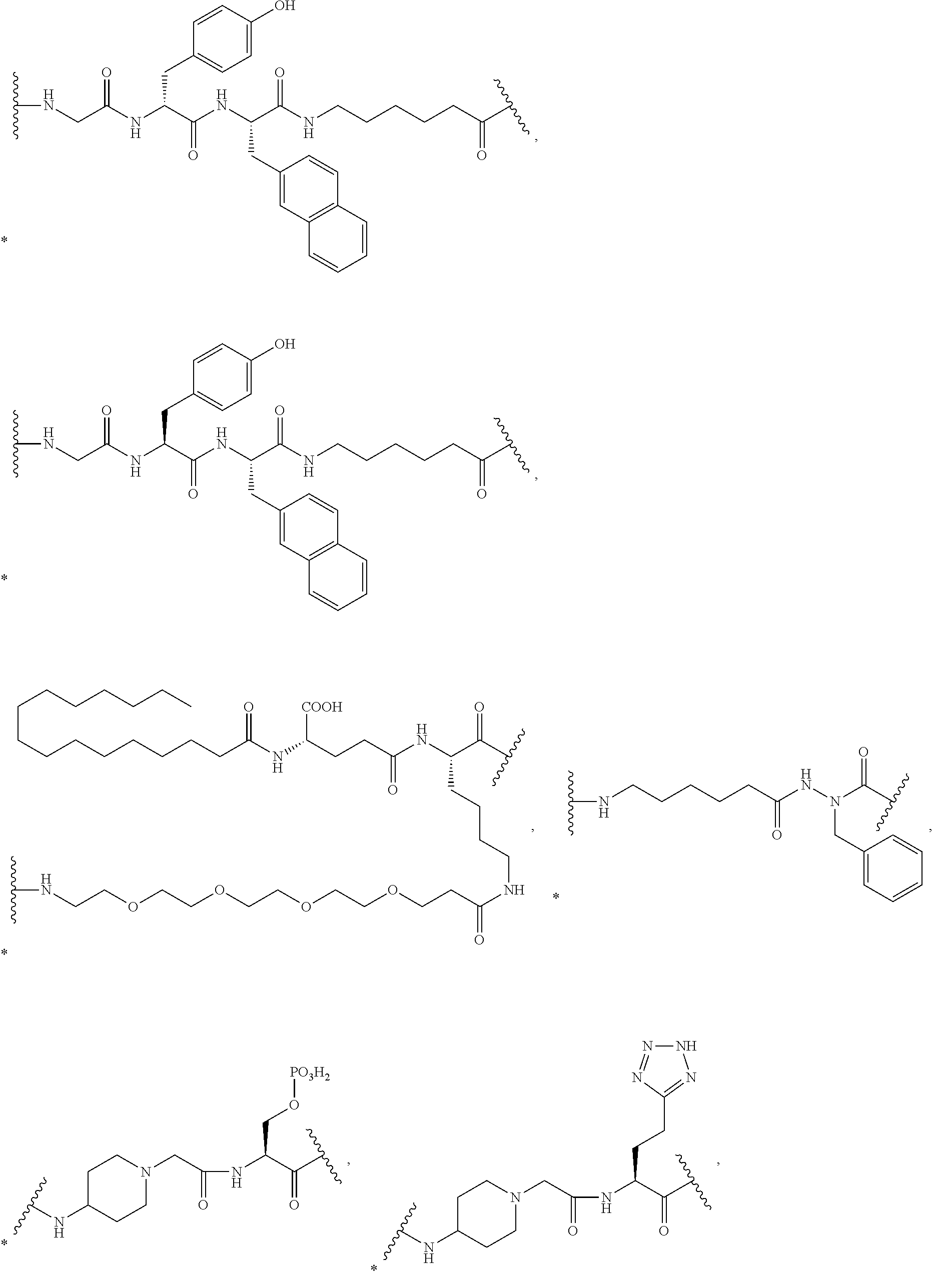 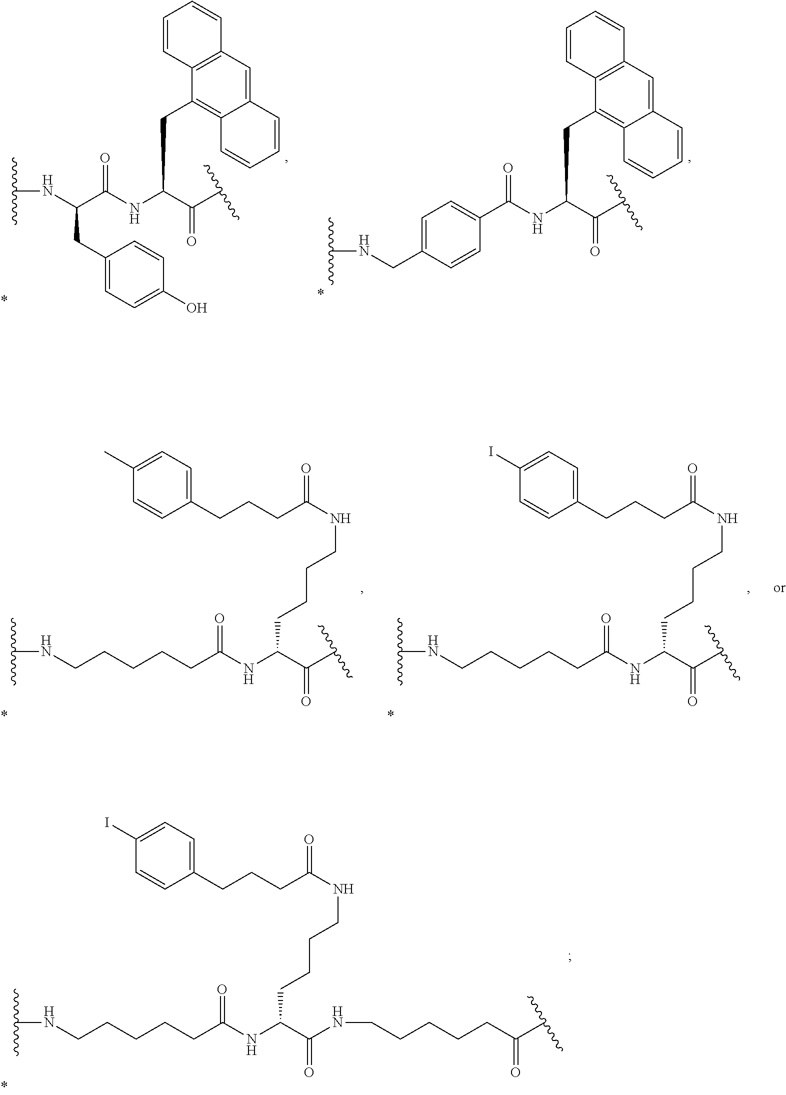 [SMILES: *N[C@H](Cc1ccc(O)cc1)C(=O)N[C@@H](Cc1c2ccccc2cc2ccccc12)C(*)=O ; *NCc1ccc(C(=O)N[C@@H](Cc2c3ccccc3cc3ccccc23)C(*)=O)cc1 ; *NCCCCCC(=O)N[C@H](CCCCNC(=O)CCCc1ccc(C)cc1)C(*)=O ; *NCCCCCC(=O)N[C@H](CCCCNC(=O)CCCc1ccc(I)cc1)C(*)=O ; *NCCCCCC(=O)N[C@H](CCCCNC(=O)CCCc1ccc(I)cc1)C(=O)NCCCCCC(*)=O]
m is 1, 2, 3, 4, 5, or 6;
z is 1, 2, 3, 4, 5, or 6;
w is 1, 2, 3, 4, 5, or 6;
wherein * denotes the attachment point to R^{a}.

22. The compound of claim 1, or a pharmaceutically acceptable salt thereof, wherein -L- is:
absent, 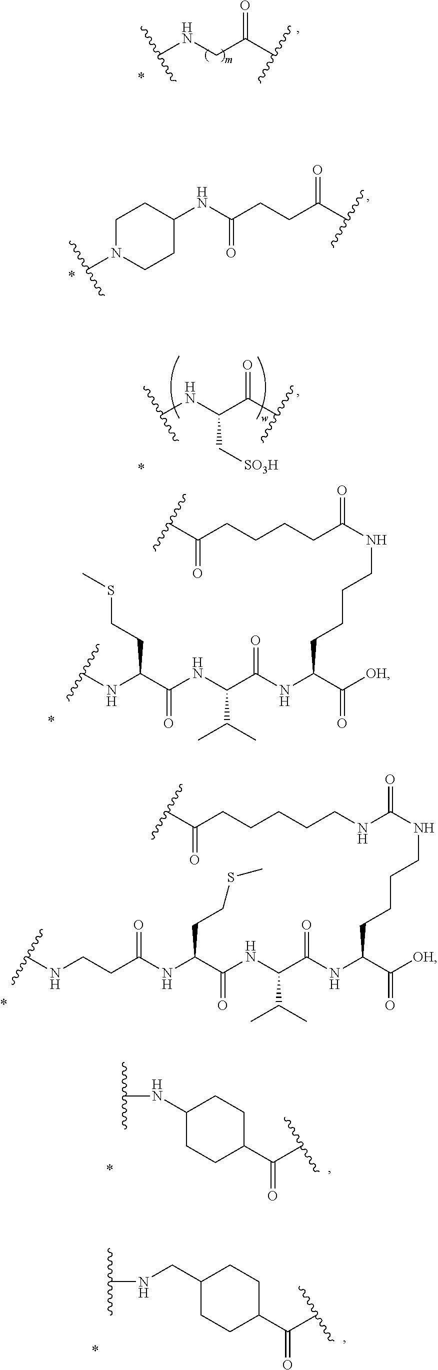 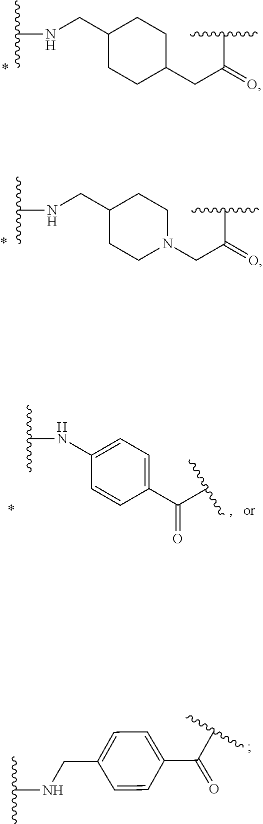 [SMILES: *NCC1CCC(CC(*)=O)CC1 ; *NCC1CCN(CC(*)=O)CC1 ; *Nc1ccc(C(*)=O)cc1 ; *NCc1ccc(C(*)=O)cc1]
wherein * denotes the attachment point to R^{a}; and
m is 1, 2, 3, 4, 5, or 6.

23. The compound of claim 1, or a pharmaceutically acceptable salt thereof, wherein: -L- is: absent,

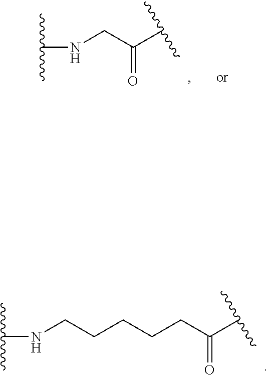 [SMILES: *NCC(*)=O ; *NCCCCCC(*)=O]

24. The compound of claim 1, or a pharmaceutically acceptable salt thereof, wherein R^{a}-L- is: 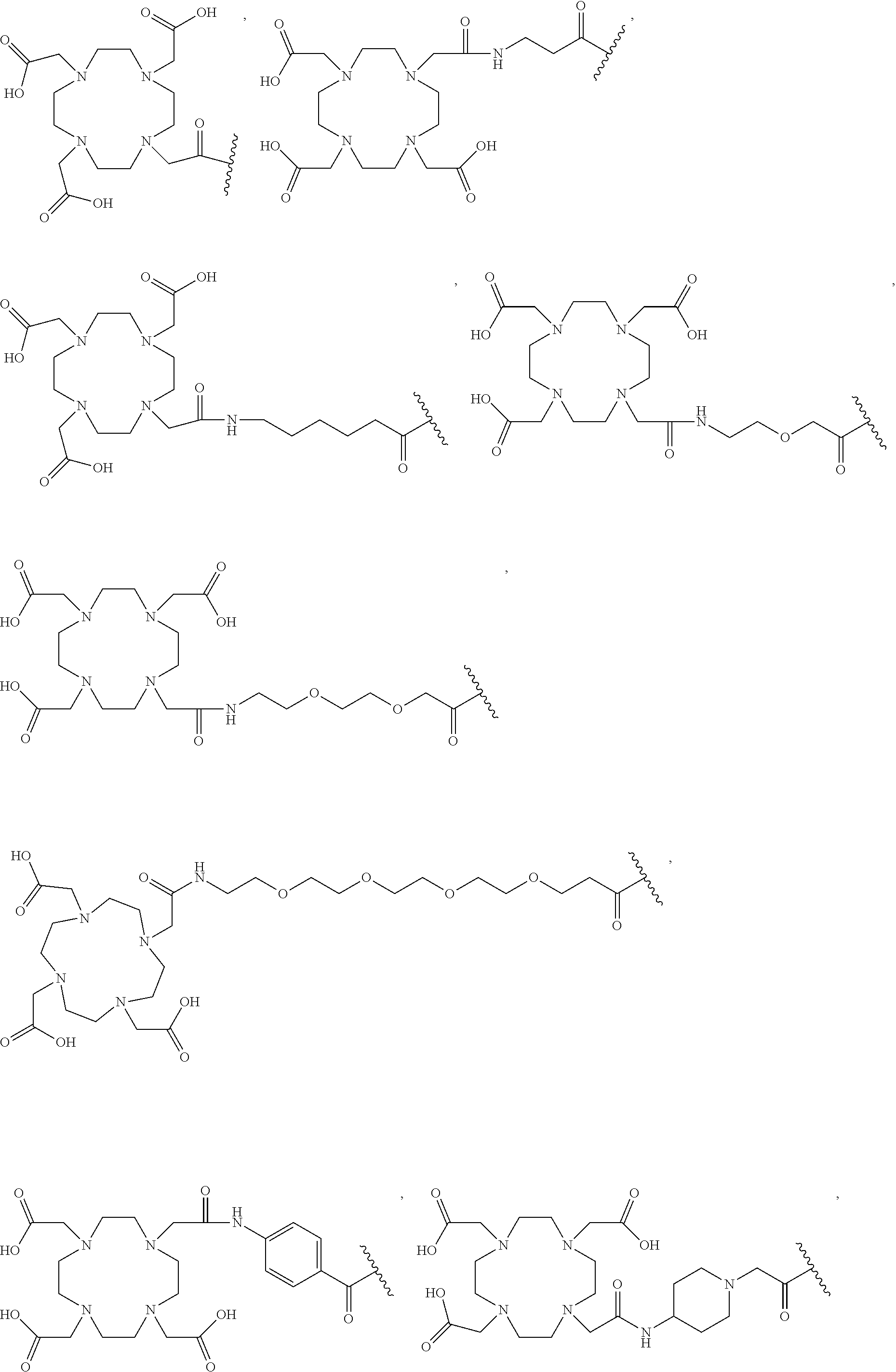 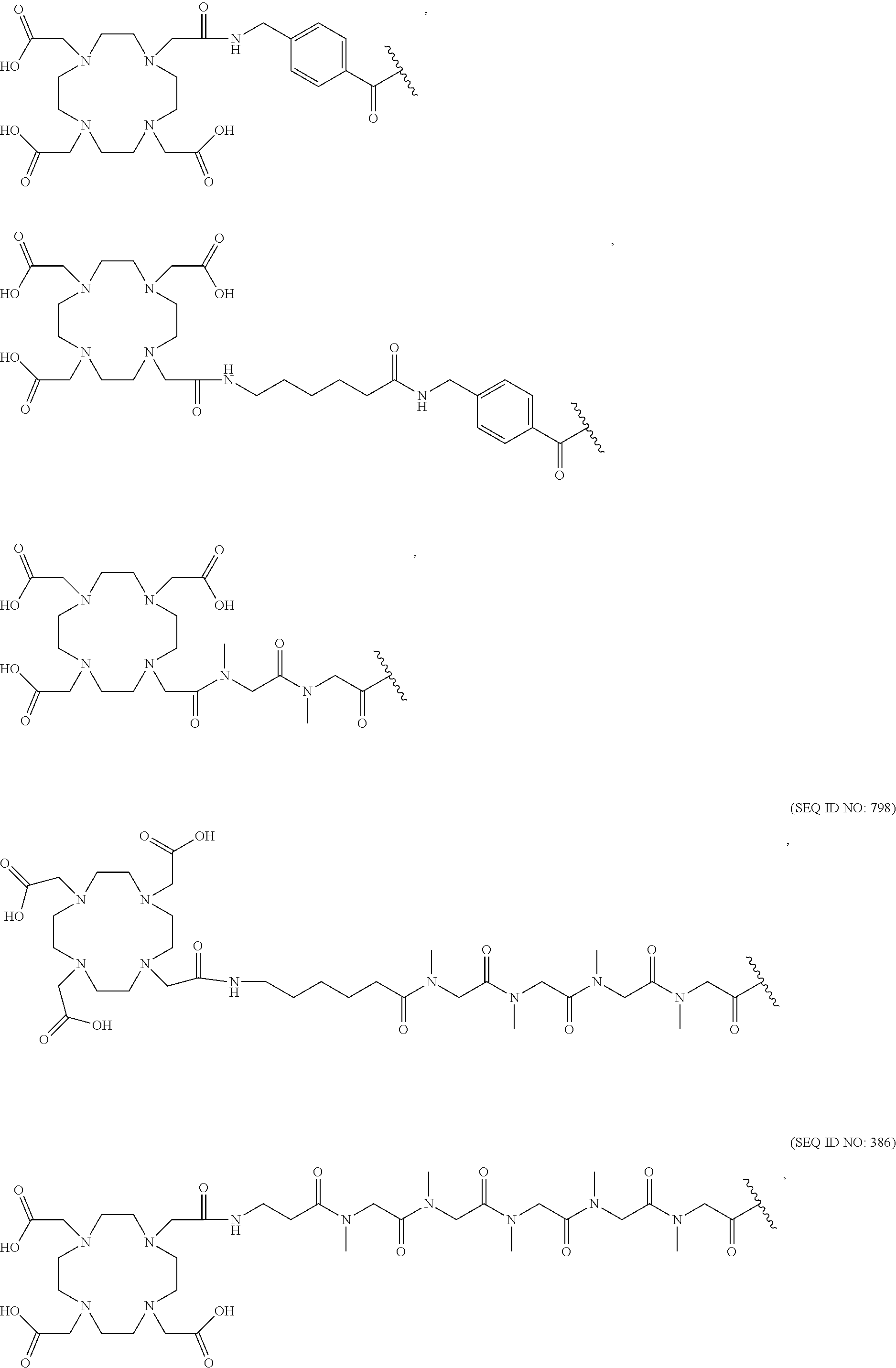 [SMILES: *C(=O)c1ccc(CNC(=O)CN2CCN(CC(=O)O)CCN(CC(=O)O)CCN(CC(=O)O)CC2)cc1 ; *C(=O)c1ccc(CNC(=O)CCCCCNC(=O)CN2CCN(CC(=O)O)CCN(CC(=O)O)CCN(CC(=O)O)CC2)cc1 ; *C(=O)CN(C)C(=O)CN(C)C(=O)CN1CCN(CC(=O)O)CCN(CC(=O)O)CCN(CC(=O)O)CC1 ; *C(=O)CN(C)C(=O)CN(C)C(=O)CN(C)C(=O)CN(C)C(=O)CCCCCNC(=O)CN1CCN(CC(=O)O)CCN(CC(=O)O)CCN(CC(=O)O)CC1 ; *C(=O)CN(C)C(=O)CN(C)C(=O)CN(C)C(=O)CN(C)C(=O)CN(C)C(=O)CCNC(=O)CN1CCN(CC(=O)O)CCN(CC(=O)O)CCN(CC(=O)O)CC1] 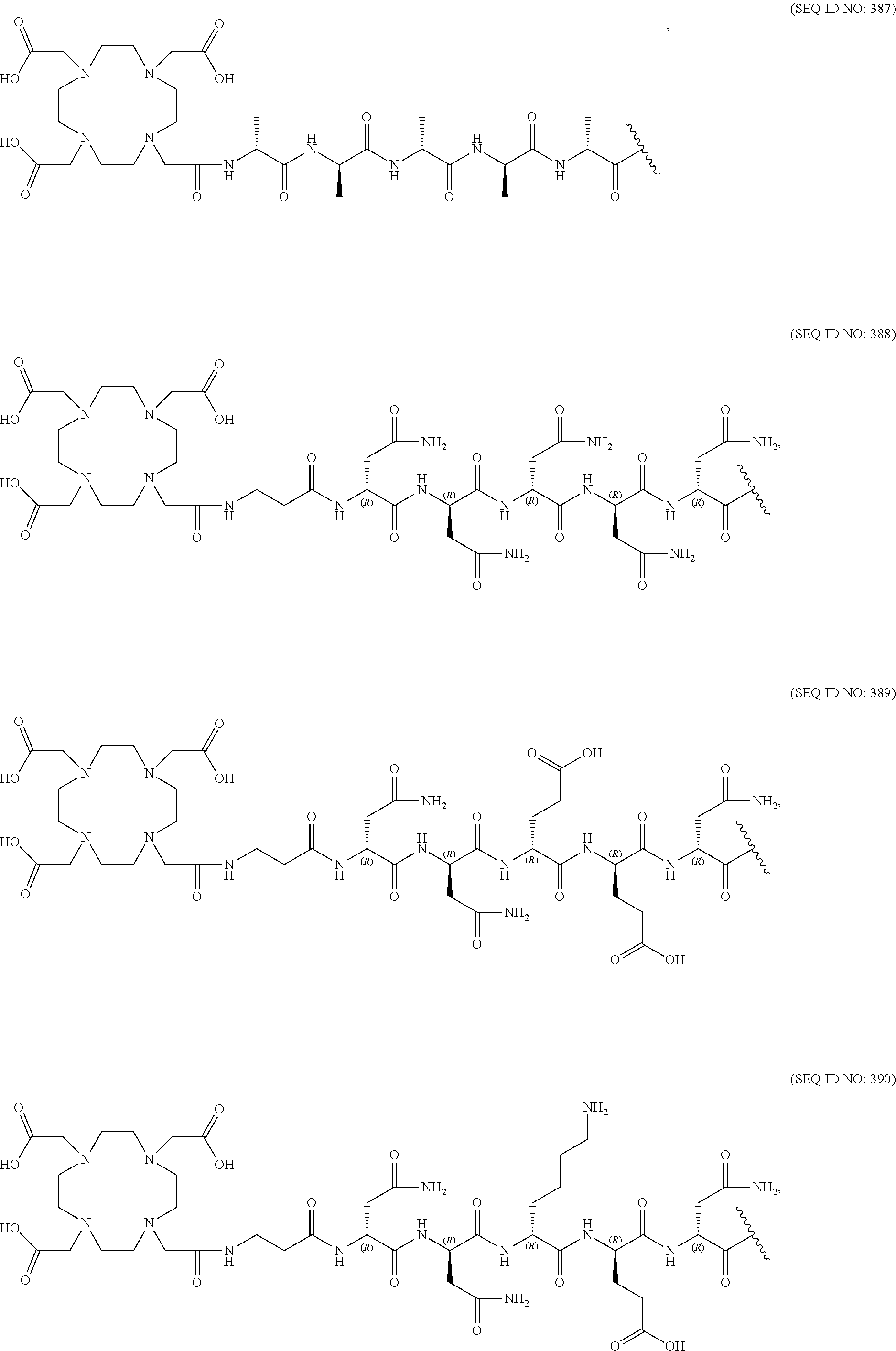 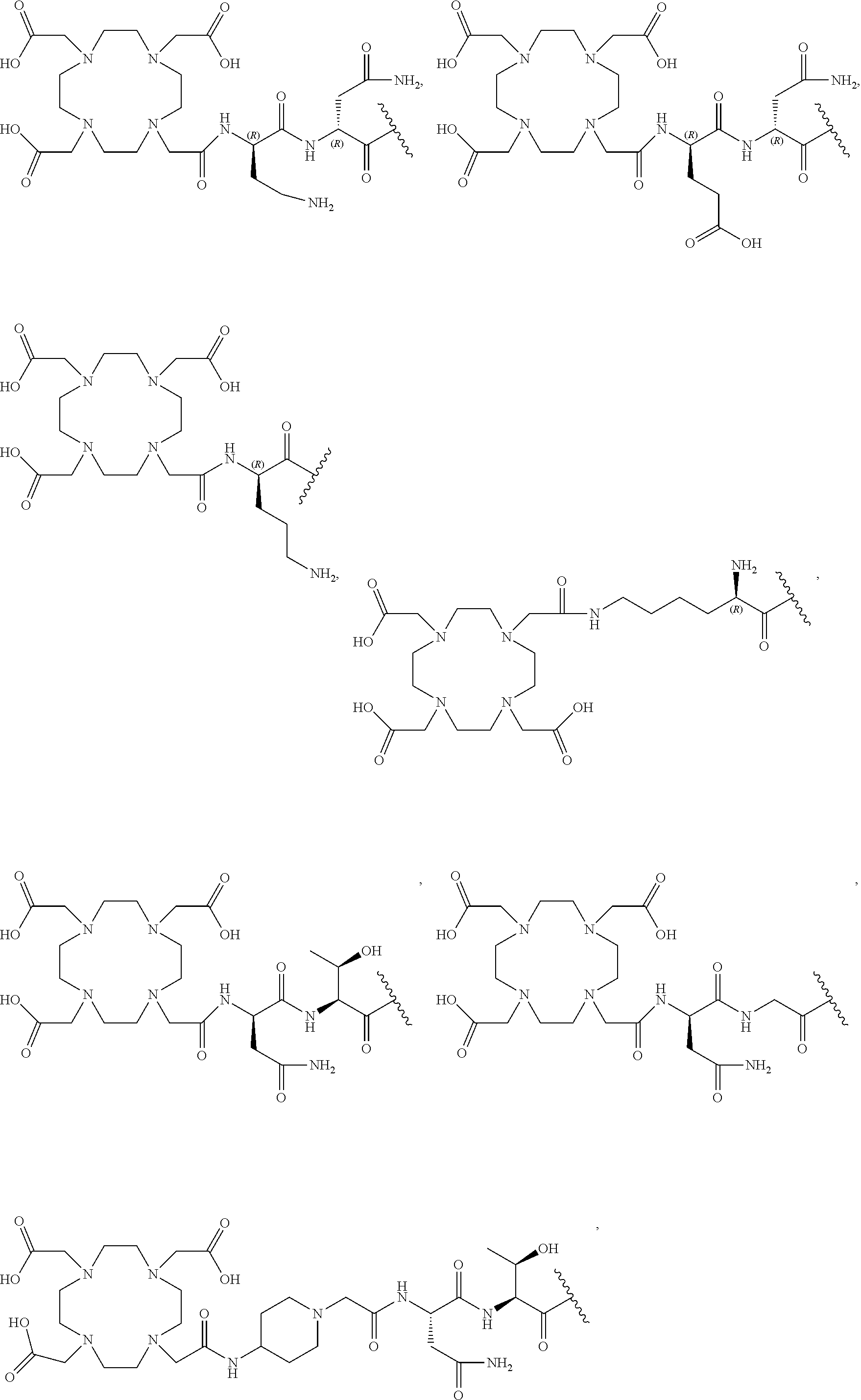 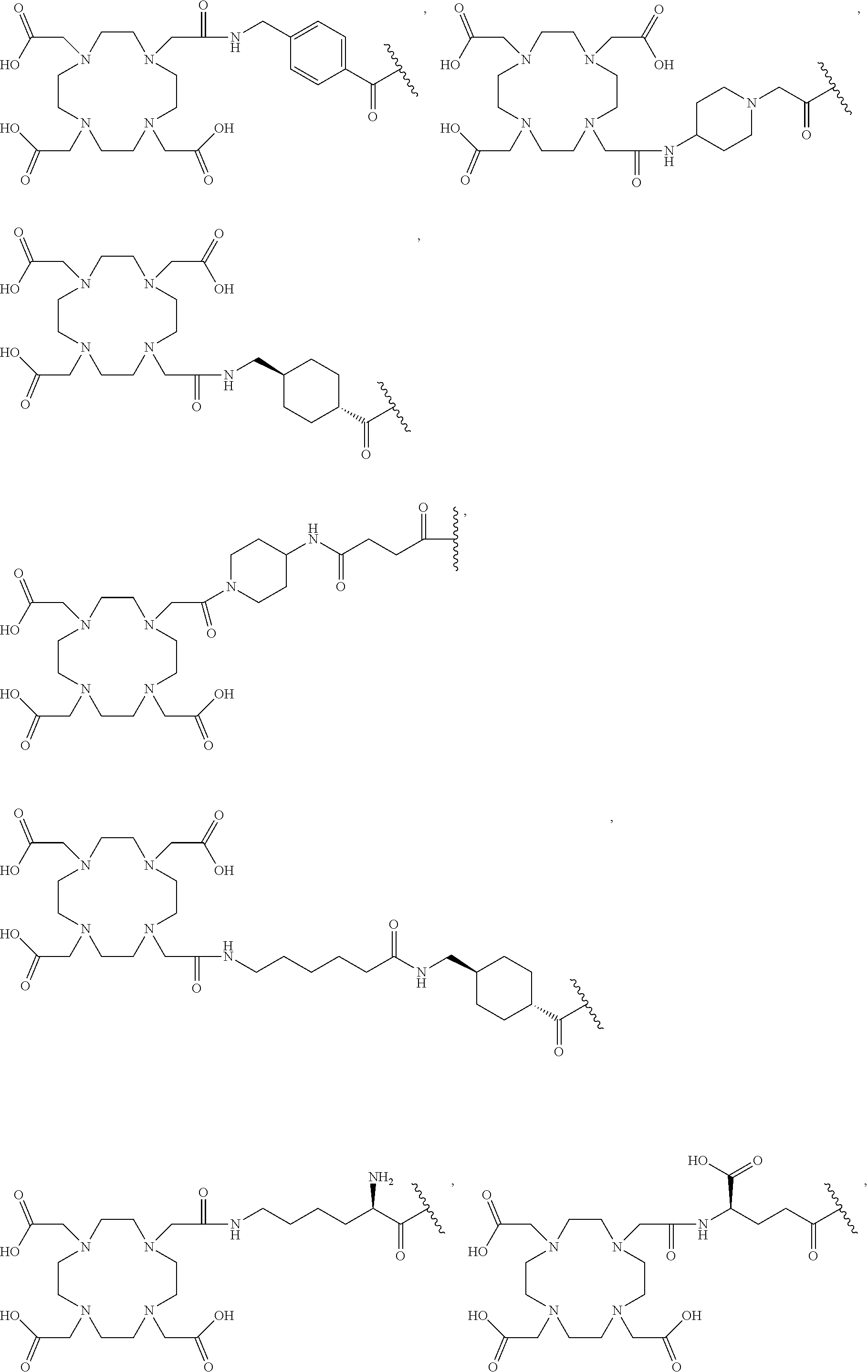 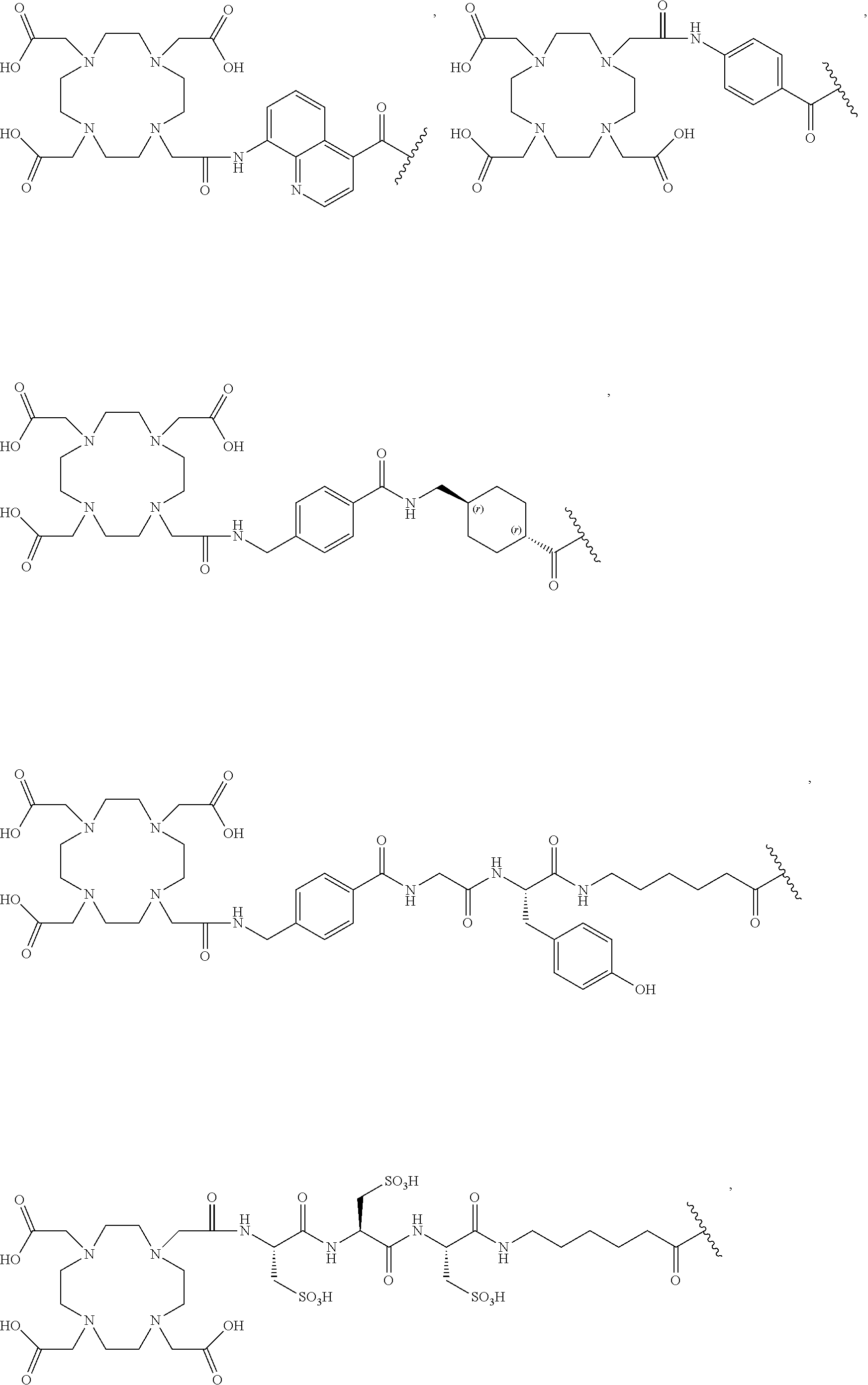 [SMILES: *C(=O)c1ccnc2c(NC(=O)CN3CCN(CC(=O)O)CCN(CC(=O)O)CCN(CC(=O)O)CC3)cccc12 ; *C(=O)c1ccc(NC(=O)CN2CCN(CC(=O)O)CCN(CC(=O)O)CCN(CC(=O)O)CC2)cc1 ; *C(=O)[C@H]1CC[C@H](CNC(=O)c2ccc(CNC(=O)CN3CCN(CC(=O)O)CCN(CC(=O)O)CCN(CC(=O)O)CC3)cc2)CC1 ; *C(=O)CCCCCNC(=O)[C@H](Cc1ccc(O)cc1)NC(=O)CNC(=O)c1ccc(CNC(=O)CN2CCN(CC(=O)O)CCN(CC(=O)O)CCN(CC(=O)O)CC2)cc1 ; *C(=O)CCCCCNC(=O)[C@H](CS(=O)(=O)O)NC(=O)[C@H](CS(=O)(=O)O)NC(=O)[C@H](CS(=O)(=O)O)NC(=O)CN1CCN(CC(=O)O)CCN(CC(=O)O)CCN(CC(=O)O)CC1] 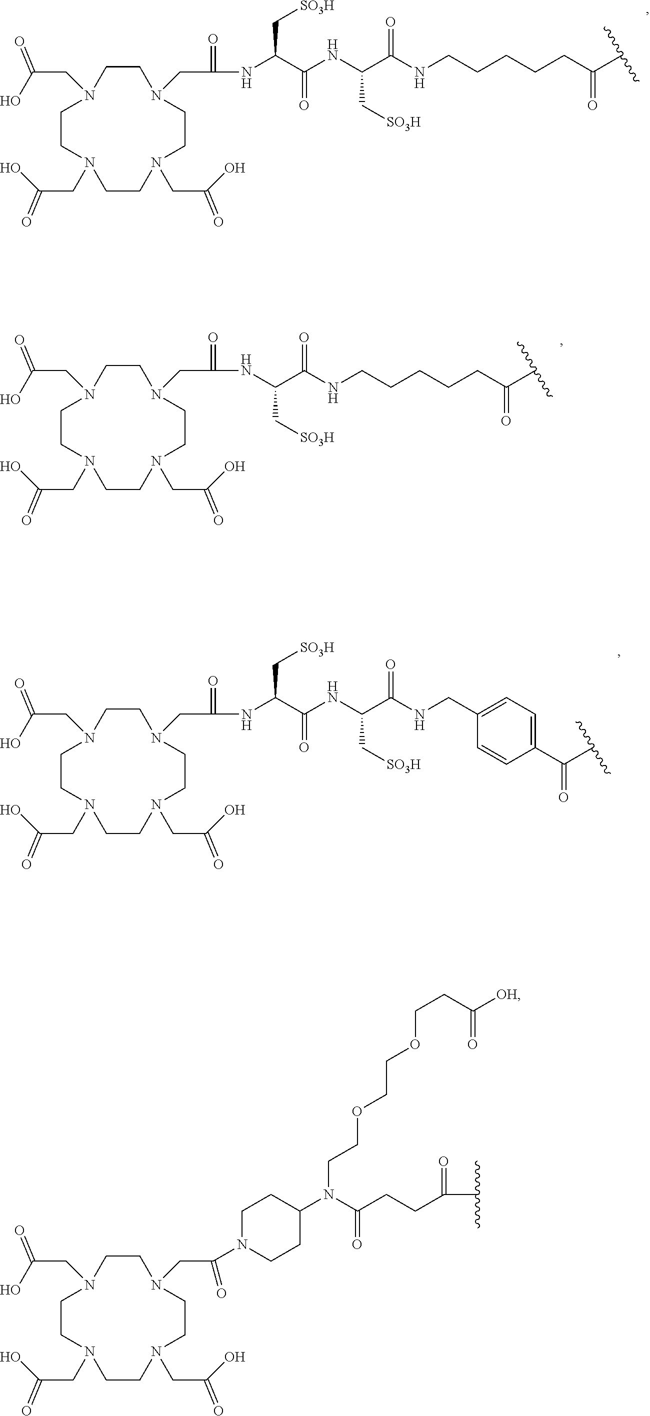 [SMILES: *C(=O)CCCCCNC(=O)[C@H](CS(=O)(=O)O)NC(=O)[C@H](CS(=O)(=O)O)NC(=O)CN1CCN(CC(=O)O)CCN(CC(=O)O)CCN(CC(=O)O)CC1 ; *C(=O)CCCCCNC(=O)[C@H](CS(=O)(=O)O)NC(=O)CN1CCN(CC(=O)O)CCN(CC(=O)O)CCN(CC(=O)O)CC1 ; *C(=O)c1ccc(CNC(=O)[C@H](CS(=O)(=O)O)NC(=O)[C@H](CS(=O)(=O)O)NC(=O)CN2CCN(CC(=O)O)CCN(CC(=O)O)CCN(CC(=O)O)CC2)cc1 ; *C(=O)CCC(=O)N(CCOCCOCCC(=O)O)C1CCN(C(=O)CN2CCN(CC(=O)O)CCN(CC(=O)O)CCN(CC(=O)O)CC2)CC1] 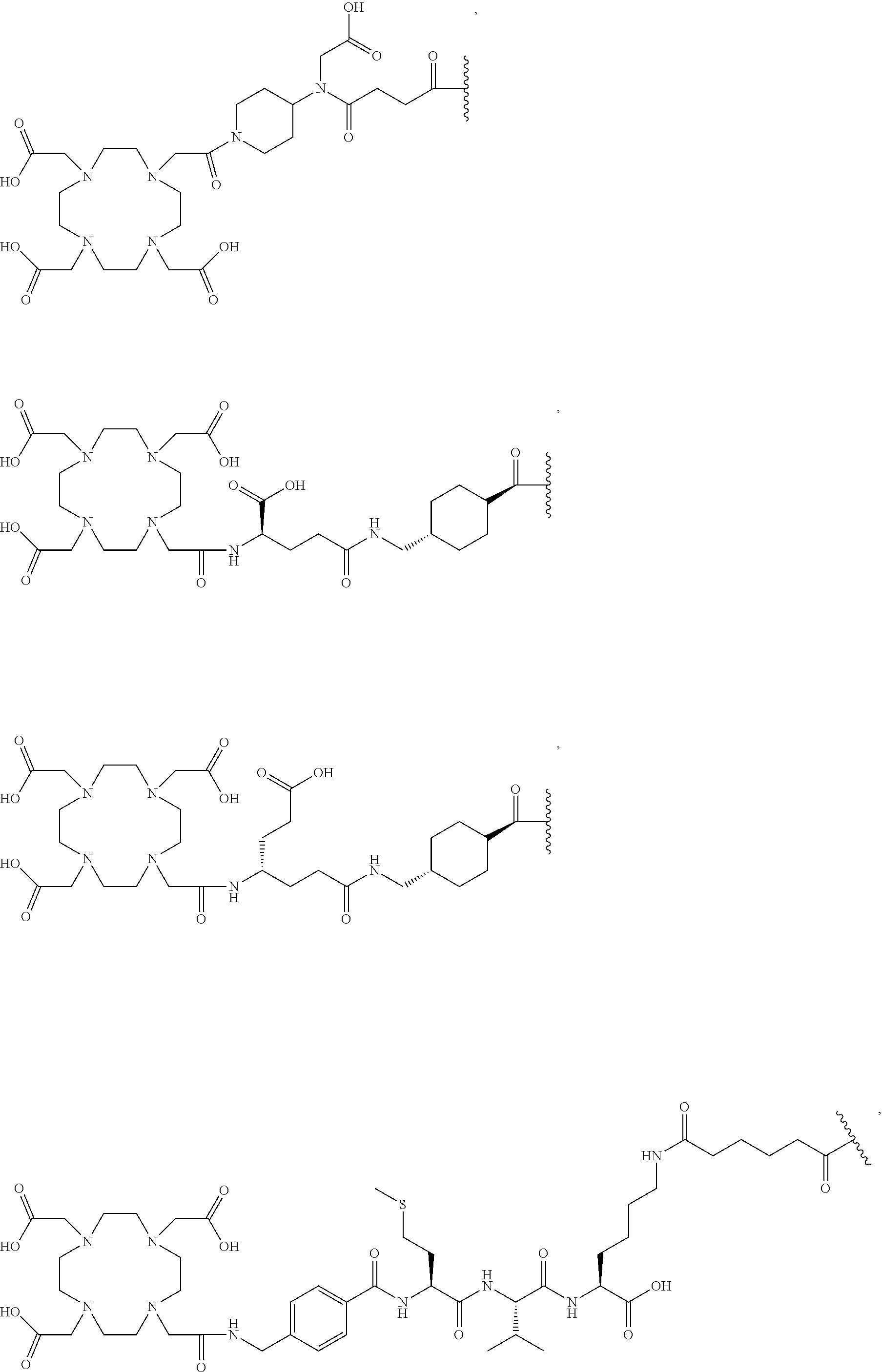 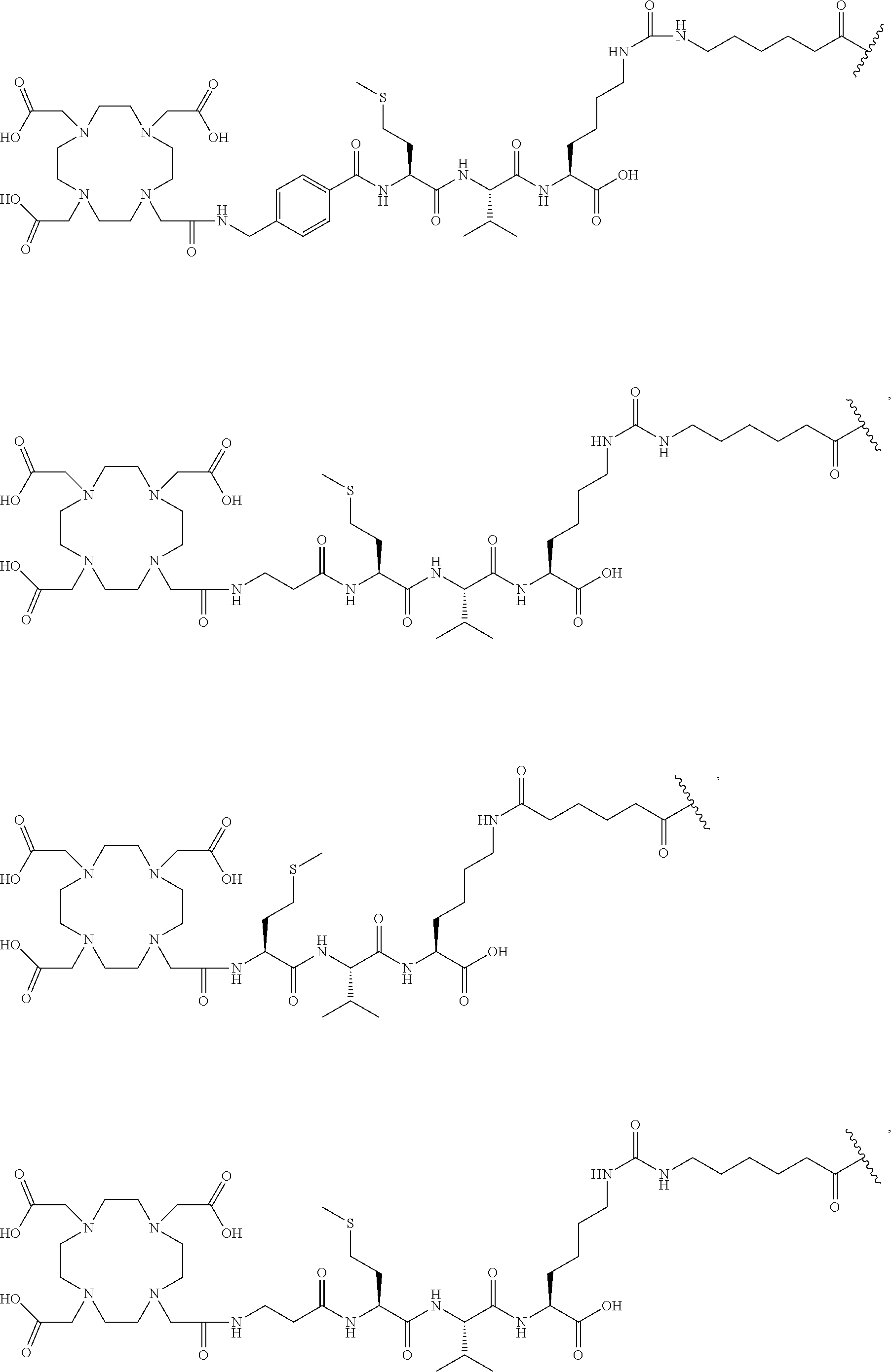 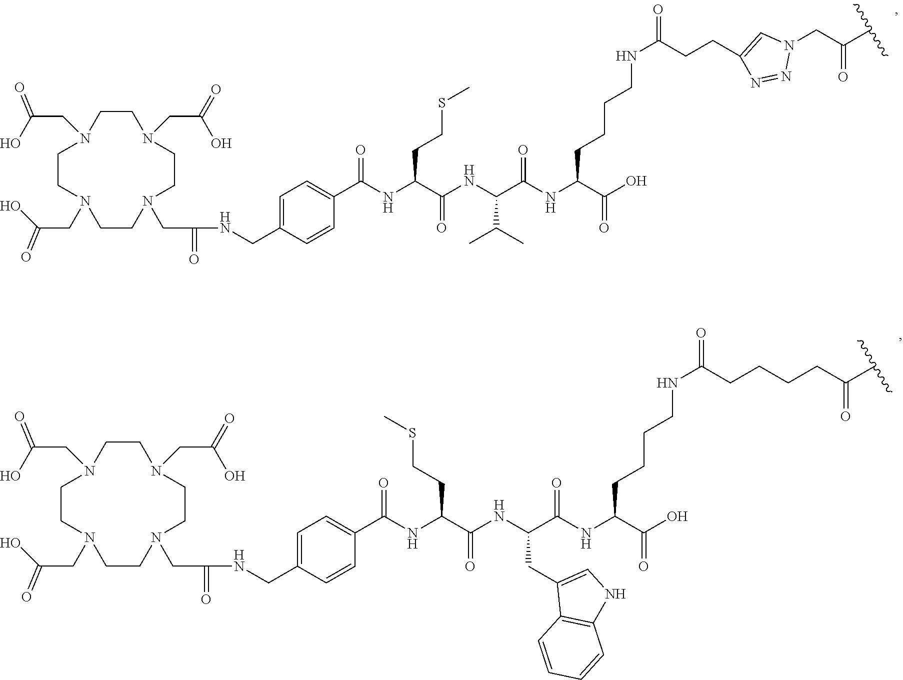 [SMILES: *C(=O)Cn1cc(CCC(=O)NCCCC[C@H](NC(=O)[C@@H](NC(=O)[C@H](CCSC)NC(=O)c2ccc(CNC(=O)CN3CCN(CC(=O)O)CCN(CC(=O)O)CCN(CC(=O)O)CC3)cc2)C(C)C)C(=O)O)nn1 ; *C(=O)CCCCC(=O)NCCCC[C@H](NC(=O)[C@H](Cc1c[nH]c2ccccc12)NC(=O)[C@H](CCSC)NC(=O)c1ccc(CNC(=O)CN2CCN(CC(=O)O)CCN(CC(=O)O)CCN(CC(=O)O)CC2)cc1)C(=O)O] 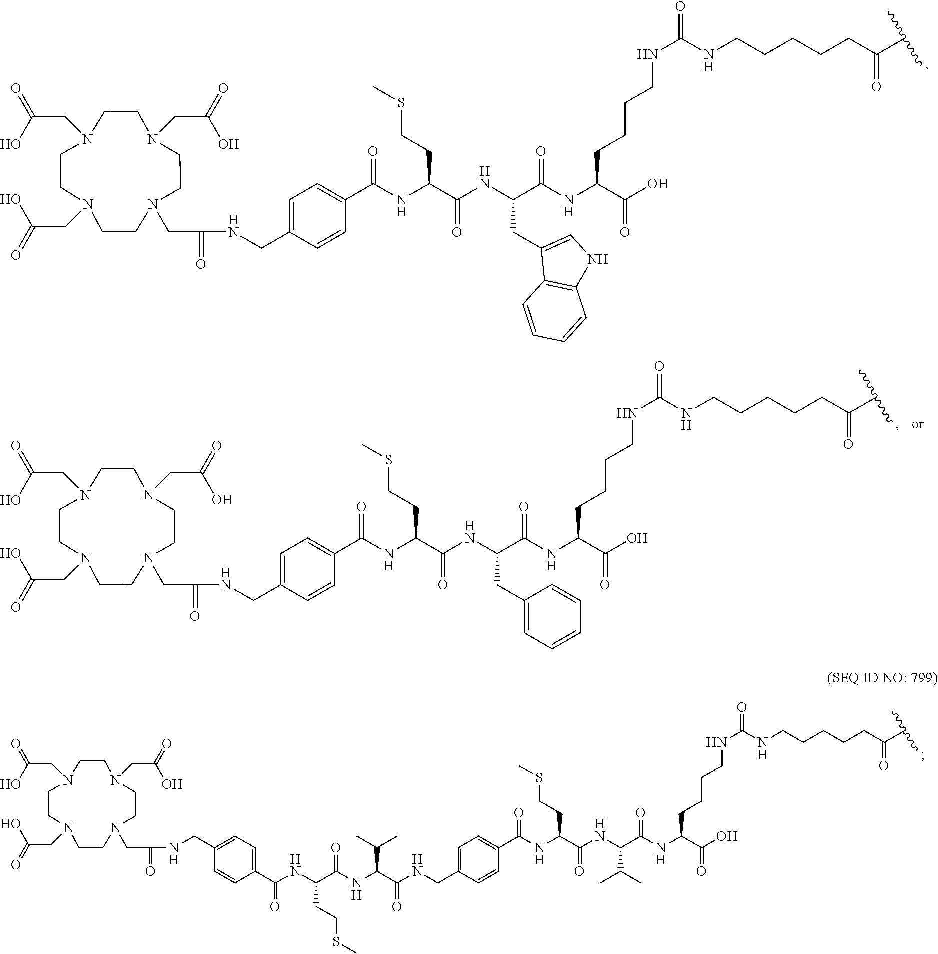 [SMILES: *C(=O)CCCCCNC(=O)NCCCC[C@H](NC(=O)[C@H](Cc1c[nH]c2ccccc12)NC(=O)[C@H](CCSC)NC(=O)c1ccc(CNC(=O)CN2CCN(CC(=O)O)CCN(CC(=O)O)CCN(CC(=O)O)CC2)cc1)C(=O)O ; *C(=O)CCCCCNC(=O)NCCCC[C@H](NC(=O)[C@H](Cc1ccccc1)NC(=O)[C@H](CCSC)NC(=O)c1ccc(CNC(=O)CN2CCN(CC(=O)O)CCN(CC(=O)O)CCN(CC(=O)O)CC2)cc1)C(=O)O ; *C(=O)CCCCCNC(=O)NCCCC[C@H](NC(=O)[C@@H](NC(=O)[C@H](CCSC)NC(=O)c1ccc(CNC(=O)[C@@H](NC(=O)[C@H](CCSC)NC(=O)c2ccc(CNC(=O)CN3CCN(CC(=O)O)CCN(CC(=O)O)CCN(CC(=O)O)CC3)cc2)C(C)C)cc1)C(C)C)C(=O)O]
or a radionuclide complex thereof.

25. The compound of claim 1, or a pharmaceutically acceptable salt thereof, wherein the compound of Formula (I) has the chemical structure corresponding to one of the following SEQ ID numbers, or a pharmaceutically acceptable salt thereof: 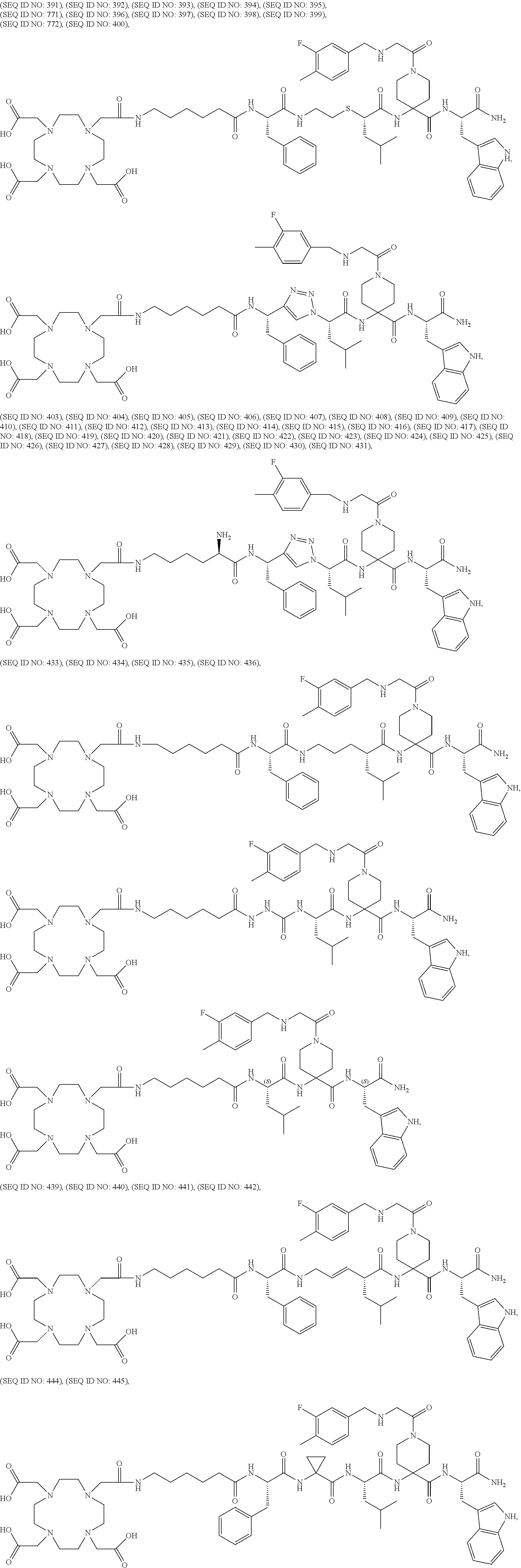 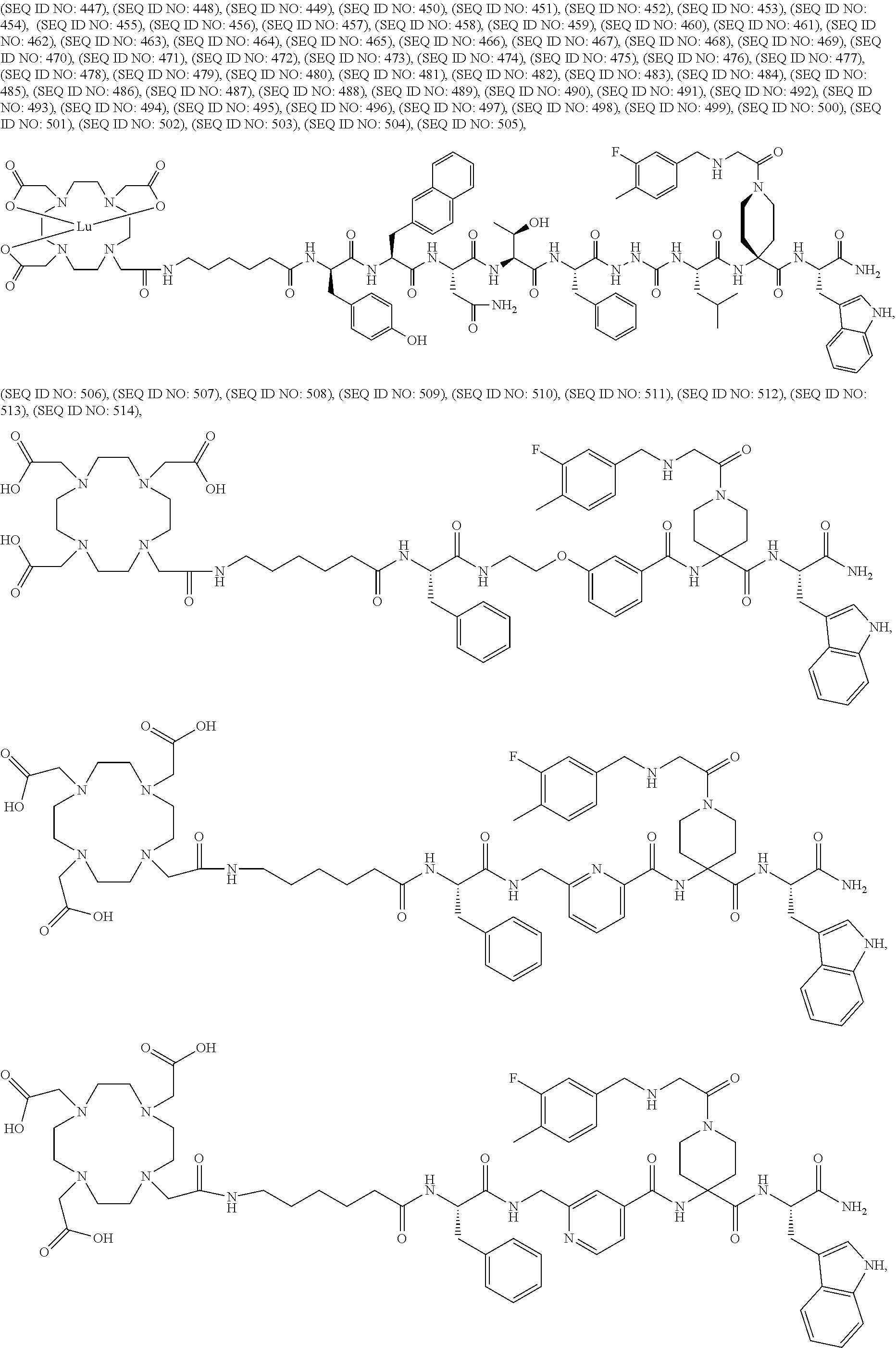 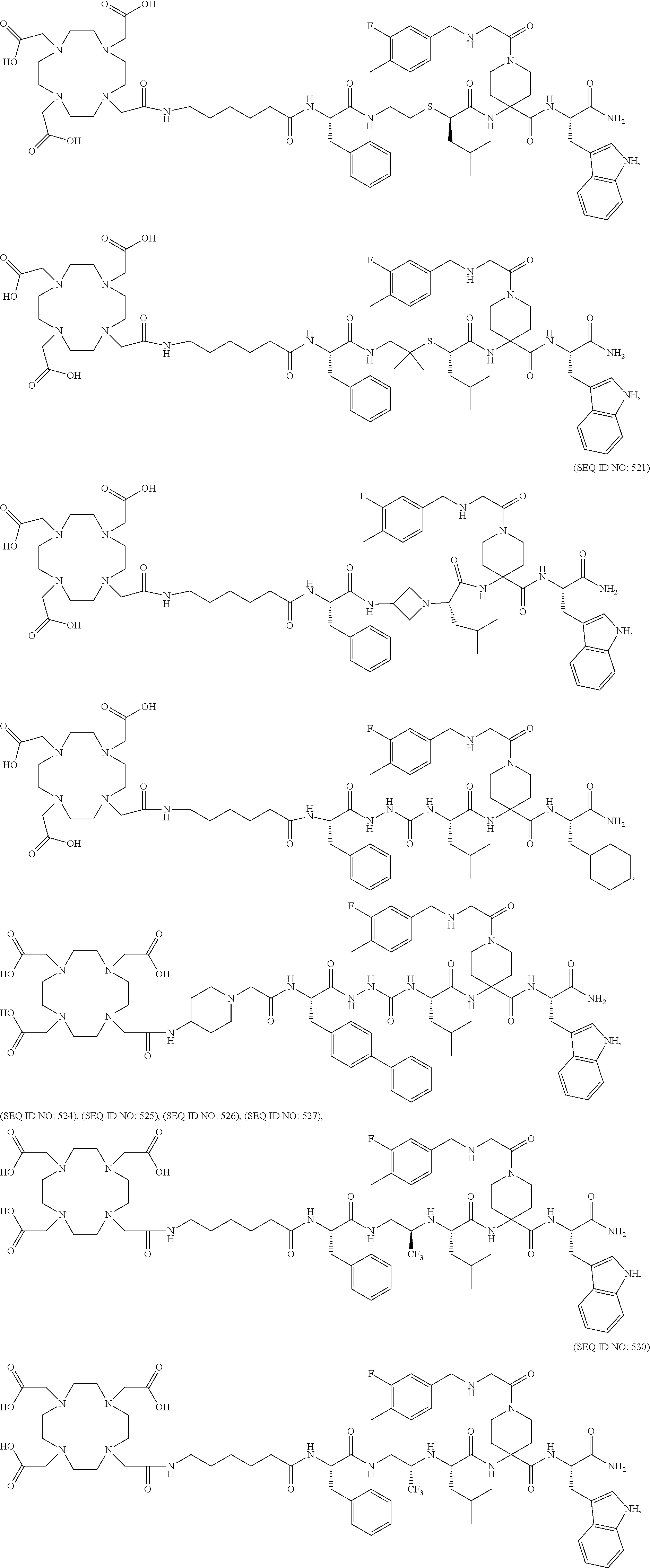 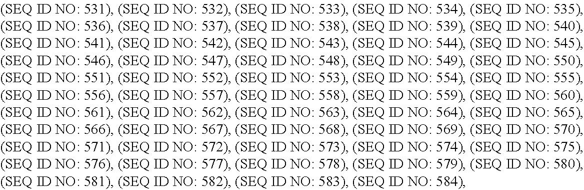 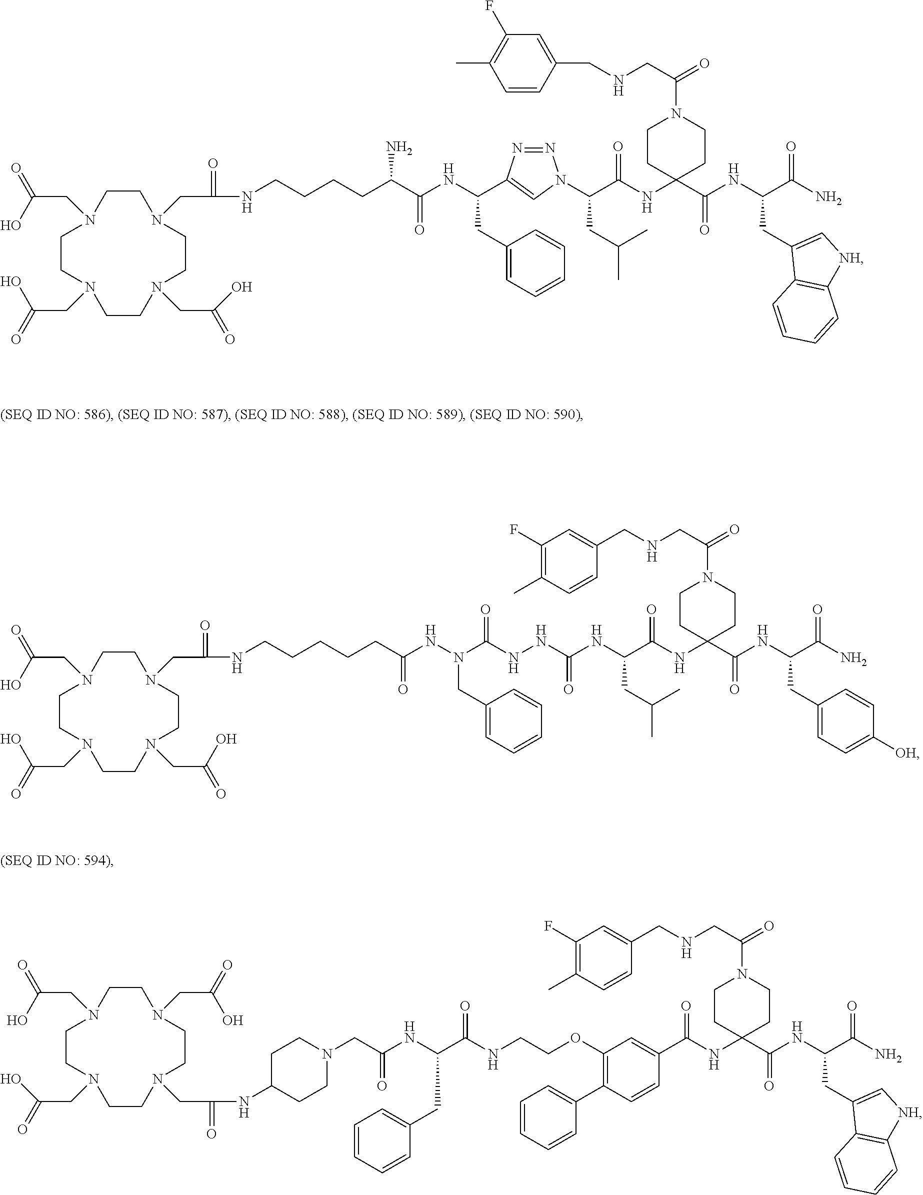 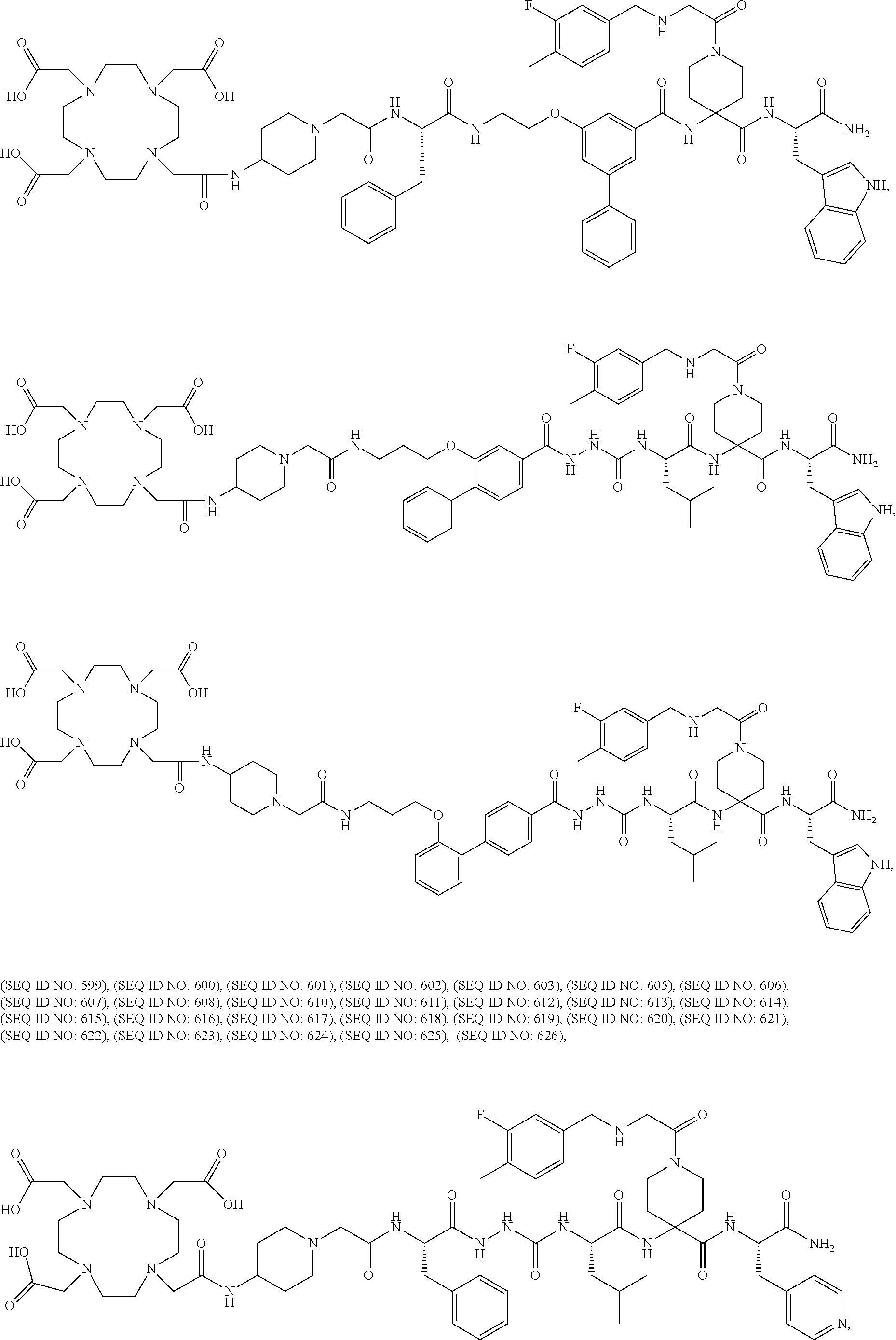 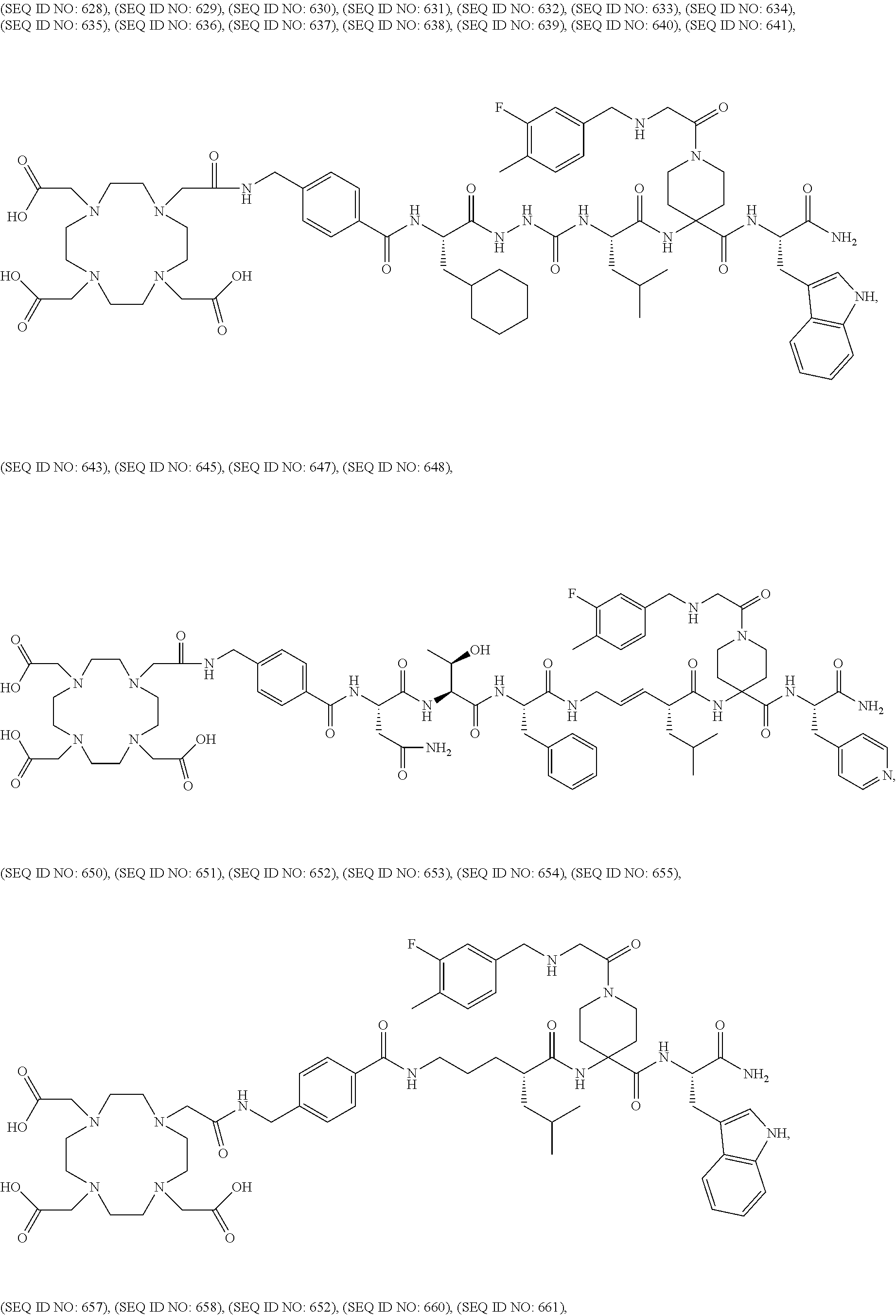 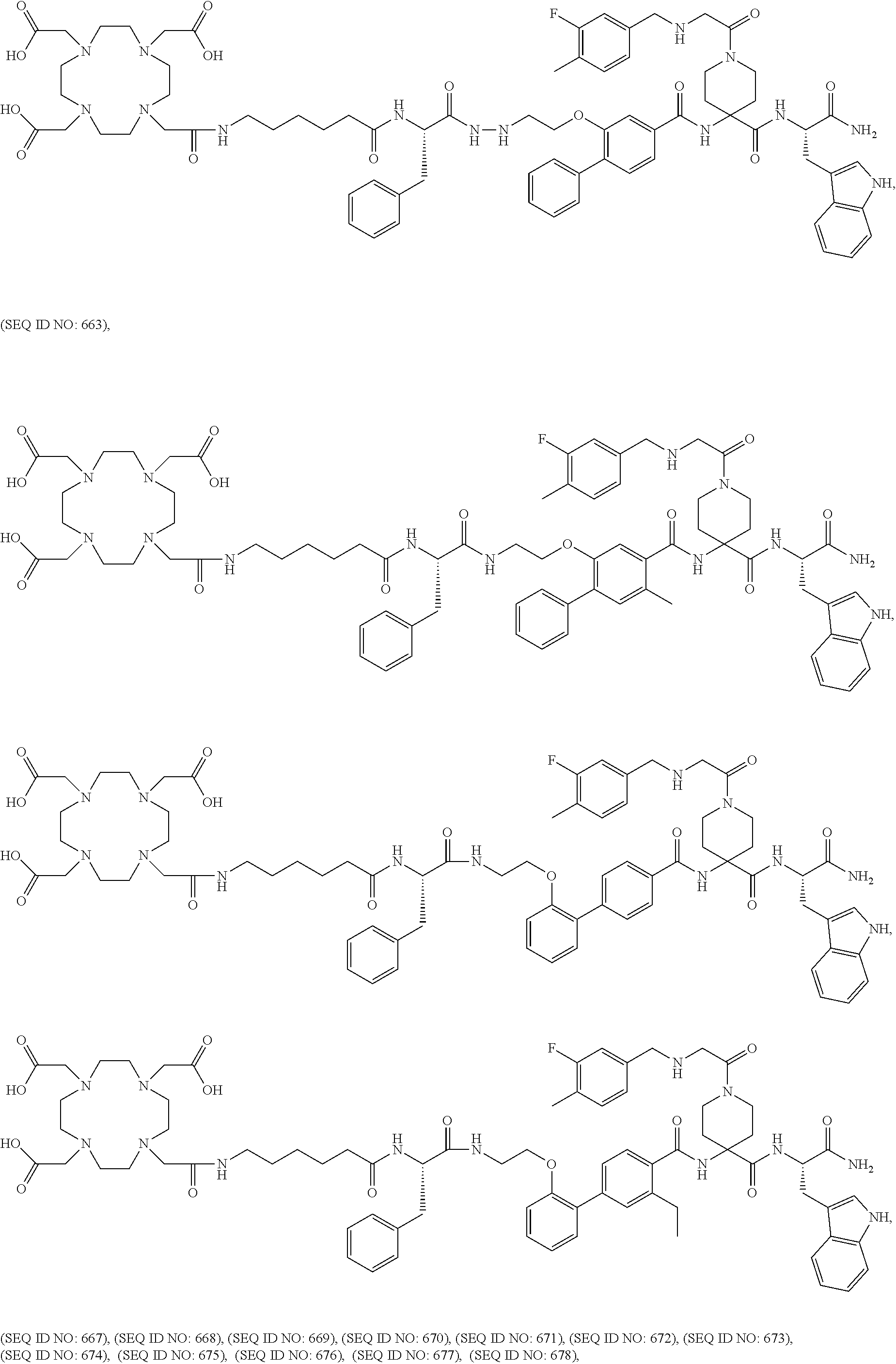 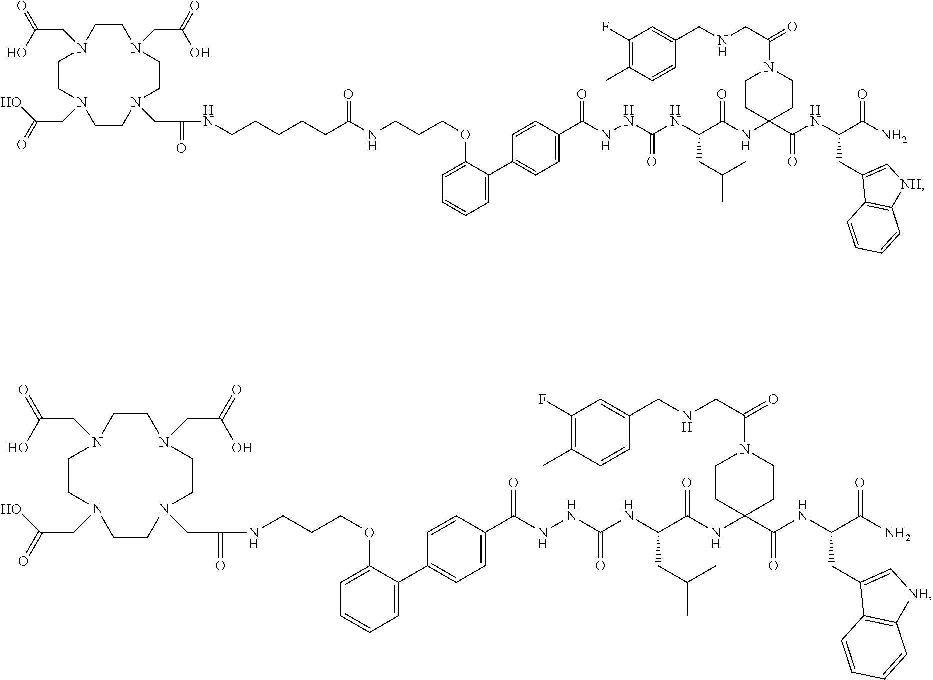 [SMILES: Cc1ccc(CNCC(=O)N2CCC(NC(=O)[C@H](CC(C)C)NC(=O)NNC(=O)c3ccc(-c4ccccc4OCCCNC(=O)CCCCCNC(=O)CN4CCN(CC(=O)O)CCN(CC(=O)O)CCN(CC(=O)O)CC4)cc3)(C(=O)N[C@@H](Cc3c[nH]c4ccccc34)C(N)=O)CC2)cc1F ; Cc1ccc(CNCC(=O)N2CCC(NC(=O)[C@H](CC(C)C)NC(=O)NNC(=O)c3ccc(-c4ccccc4OCCCNC(=O)CN4CCN(CC(=O)O)CCN(CC(=O)O)CCN(CC(=O)O)CC4)cc3)(C(=O)N[C@@H](Cc3c[nH]c4ccccc34)C(N)=O)CC2)cc1F] 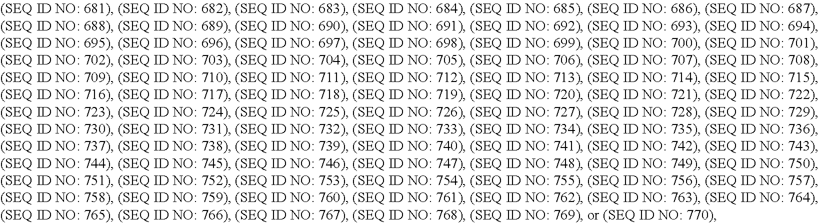
or a radionuclide complex thereof;
wherein the radionuclide of the radionuclide complex is an Auger electron-emitting radionuclide, α-emitting radionuclide, β-emitting radionuclide, or γ-emitting radionuclide.

26. The compound of claim 1, or a pharmaceutically acceptable salt thereof, wherein: the compound is a radionuclide complex and the radionuclide of the radionuclide complex is 111-indium (¹¹¹In), 67-gallium (⁶⁷Ga), 68-gallium (⁶⁸Ga), 69-gallium (⁶⁹Ga), 71-gallium (71Ga), 225-actinium (²²⁵Ac), 175-lutetium (¹⁷⁵Lu), 177-lutetium (¹⁷⁷Lu), 204-lead (²⁰⁴Pb), 206-lead (²⁰⁶Pb), 207-lead (²⁰⁷Pb), 208-lead (²⁰⁸Pb), 212-lead (²¹⁴Pb), 63-copper (⁶³Cu), 64-copper (64Cu), 65-copper (⁶⁵Cu), or 67-copper (⁶⁷Cu)

27. The compound of claim 1, or a pharmaceutically acceptable salt thereof, wherein: the compound is a radionuclide complex and the radionuclide of the radionuclide complex is 64-copper (⁶⁴Cu), 67-copper (⁶⁷Cu), 90-yttrium (⁹⁰Y), 111-indium (¹¹¹In), 67-gallium (⁶⁷Ga), 68-gallium (⁶⁸Ga), 225-actinium (²²⁵Ac), or 177-lutetium (¹⁷⁷Lu) or 212-lead (²¹²Pb).

28. A pharmaceutical composition comprising a compound of claim 1, or a pharmaceutically acceptable salt thereof, and at least one pharmaceutically acceptable excipient.

29. A method for the treatment of cancer comprising administering to a mammal with cancer an effective amount of a compound of claim 1, or a pharmaceutically acceptable salt thereof; wherein the compound is a radionuclide complex and the radionuclide of the radionuclide complex is:
an α-emitting radionuclide that is 225-actinium (²²⁵Ac), 213-bismuth (²¹³Bi), 223-radium (²²³Ra), or 212-lead (²²¹Pb); or
a β-emitting radionuclide that is 90-yttrium (⁹⁰Y), 177-lutetium (¹⁷⁷Lu), 64-copper (⁶⁴Cu), 67-copper (⁶⁷Cu), or 153-samarium (¹⁵³Sm);
wherein the cancer is glioma, thyroid cancer, lung cancer, colorectal cancer, stomach cancer, liver cancer, pancreatic cancer, renal cancer, prostate cancer, testis cancer, breast cancer, cervical cancer, endometrial cancer, ovarian cancer, or melanoma.

30. A method of killing tumors in a mammal that overexpress Kisspeptin receptor (KISS1R) comprising administering to the mammal with tumors a compound of claim 1, or a pharmaceutically acceptable salt thereof, wherein the compound is a radionuclide complex and the radionuclide of the radionuclide complex is:
an α-emitting radionuclide that is 225-actinium (²²⁵Ac), 213-bismuth (²¹³Bi), 223-radium (²²³Ra), or 212-lead (²¹²Pb); or
a β-emitting radionuclide that is 90-yttrium (⁹⁰Y), 177-lutetium (¹⁷⁷Lu), 64-copper (⁶⁴Cu), 67-copper (⁶⁷Cu), or 153-samarium (¹⁵³Sm);
wherein the tumor is glioma, thyroid cancer, lung cancer, colorectal cancer, stomach cancer, liver cancer, pancreatic cancer, renal cancer, prostate cancer, testis cancer, breast cancer, cervical cancer, endometrial cancer, ovarian cancer, or melanoma.
